(19) United States
(12) Patent Application Publication
(10) Pub. No.: US 2026/0159512 A1
(43) Pub. Date: Jun. 11, 2026

(54) BCAT2 INHIBITORS

(71) Applicant: AGIOS PHARMACEUTICALS, INC., Cambridge, MA (US)

(72) Inventors: Claude Barberis, Ayer, MA (US); Peng Liu, Belmont, MA (US)

(21) Appl. No.: 18/709,121

(22) PCT Filed: Nov. 11, 2022
(86) PCT No.: PCT/US22/49657

§ 371 Date: May 10, 2024

Related U.S. Application Data
(60) Provisional application No. 63/278,857, filed on Nov. 12, 2021.
(60) Provisional application No. 63/359,230, filed on Jul. 8, 2022.

(51) Int. Cl. C07D 471/04; A61K 31/427; A61K 31/437; A61K 31/4439; A61K 31/444; A61K 31/454; A61K 31/4545; A61K 31/506; A61K 31/52; A61K 31/5377; C07D 409/14; C07D 413/14; C07D 417/14; C07D 473/32

(52) U.S. Cl. CPC C07D 471/04; A61K 31/427; A61K 31/437; A61K 31/4439; A61K 31/444; A61K 31/454; A61K 31/4545; A61K 31/506; A61K 31/52; A61K 31/5377; C07D 409/14; C07D 413/14; C07D 417/14; C07D 473/32

(57) ABSTRACT

The disclosure is directed to compounds, and pharmaceutically acceptable salts thereof, that inhibit branched-chain amino acid aminotransferase 2 (BCAT2), pharmaceutical compositions comprising those compounds, and methods of using those compounds for treating branched chain amino acid (BCAA)-catabolism-related diseases.

### CROSS-REFERENCE TO RELATED APPLICATIONS

This application claims the benefit of U.S. Provisional Application No. 63/278,857, filed on Nov. 12, 2021, and of U.S. Provisional Application No. 63/359,230, filed on Jul. 8, 2022, each of which is incorporated by reference herein in its entirety.

### TECHNICAL FIELD

This application pertains to compounds that inhibit branched-chain amino acid aminotransferase 2 (BCAT2), pharmaceutical compositions comprising those compounds, and methods of using those compounds for treating branched chain amino acid (BCAA)-catabolism-related diseases.

### BACKGROUND

Leucine, isoleucine, and valine are essential branched chain amino acids (BCAAs). BCAAs are catabolized by enzymatic conversions that result in transformation of these amino acids into various metabolites. FIG. 1 shows the catabolism pathways for leucine, isoleucine, and valine.

When genetic mutations result in defects in the enzymes that facilitate the BCAA catabolic conversions (e.g., loss of function mutations), toxic levels of catabolic intermediates accumulate, resulting in organic acidemias. Propionic acidemia, for example, results from loss of function mutations in propionyl-CoA carboxylase. Methylmalonic acidemia results from deficiency of methylmalonyl CoA mutase, defective enzymes (MMADHC or MMAA/B) used in the synthesis of cofactor 5′-adenosylcobalamin, or deficient activity of methylmalonyl-CoA epimerase. Maple syrup urine disease (MSUD), isovaleric acidemia (IVA), propionic acidemia (PA), and methylmalonic acidemia (MMA) are some of the most common organic acidemias.

The accumulation of toxic levels of catabolic intermediates lead to a variety of pathologic disease phenotypes. For example, acute metabolic decompensations (AMDs) are induced as a result of a catabolic stressor which triggers protein catabolism and increases the production of these toxic metabolites, resulting in additional clinical distress. AMDs contribute to the neurological deficits in patients and put them at risk of stroke, coma, and death. Patients with MMA and PA, for example, experience frequent episodes of metabolic decompensations, especially in the first years of life. Those patients can suffer from a number of complications and impairments: potentially life-threatening episodes of acidosis, hyperammonemia, pancreatitis, metabolic stroke, cardiomyopathy, bone marrow suppression, seizures, and encephalopathy (which can accompany acutely deranged metabolism). The long-term cognitive outcome of patients with PA and MMA is negatively correlated to the number of metabolic decompensations they experience. Patients with PA also often experience significant cardiac issues. Intellectual impairment can be a major secondary presentation of both PA and MMA. Convulsions are a hallmark neurological alteration seen in children with MMA. Patients with MMA also often experience renal insufficiency.

There is currently no approved therapy on the market for either PA or MMA, except for CARBAGLU®, which is approved to treat hyperammonemia seen in MMA and PA. Liver transplantation may protect against metabolic instability but is not curative and patients remain at risk for long-term complications. Hepatic-specific gene therapy is the main treatment modality under development, but is not expected to be curative because it relies on the same mechanism of action as liver transplantation. Thus, a need exists for effective therapies for treating PA, MMA, and other BCAA-catabolism-related diseases.

The first step in BCAA catabolism is common to the catabolism of each of leucine, isoleucine, and valine (the BCAAs), i.e., an enzymatic conversion mediated by BCAA aminotransferase 2 (BCAT2). Subsequent catabolism of the metabolites follows distinct pathways. Inhibition of BCAT2 will lower BCAA pathway flux, and should significantly reduce the level of toxic metabolites seen in patients with BCAA-catabolism-related diseases. Thus, a need exists for effective BCAT2 inhibitors for treating BCAA-catabolism-related diseases.

### SUMMARY

The disclosure provides compounds that are BCAT2 inhibitors, pharmaceutical compositions comprising those BCAT2 inhibitors, and methods of treating BCAA-catabolism-related diseases using those BCAT2 inhibitors.

In some aspects, the disclosure is directed to compounds of Formula IA or Formula IB:

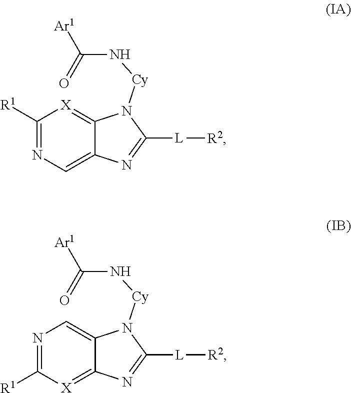

or a pharmaceutically acceptable salt thereof, wherein:
R¹ is optionally substituted heteroaryl, optionally substituted aryl, or optionally substituted 4-, 5- or 6-membered heterocycloalkyl;
X is CH or N;
Cy is optionally substituted C₅-C₇ cycloalkyl or optionally substituted 6- or 7-membered heterocycloalkyl;
L is a bond, —O—, —NR^{a}—, or optionally substituted C₁-C₄alkylene;
   R^{a} is hydrogen or C₁-C₄alkyl;
R² is optionally substituted phenyl, optionally substituted pyridinyl, optionally substituted 5-membered heteroaryl, optionally substituted heterocycloalkyl, optionally substituted C₁-C₆alkyl, optionally substituted C₁-C₆alkoxy, optionally substituted C₁-C₆hydroxyalkyl, optionally substituted C₁-C₆haloalkyl, optionally substituted C₁-C₆haloalkoxy, optionally substituted C₃-C₆cycloalkyl, or NR^{f}R^{g};
   R^{f} and R^{g} are each independently hydrogen, C₁-C₆alkyl, C₁-C₆haloalkyl, OH, C₁-C₄alkoxy, C₁-C₆hydroxyalkyl, or C₃-C₆cycloalkyl; and
Ar¹ is an optionally substituted five-membered heteroaryl.

In some aspects, the disclosure is directed to compounds of Formula IIA or Formula IIB:

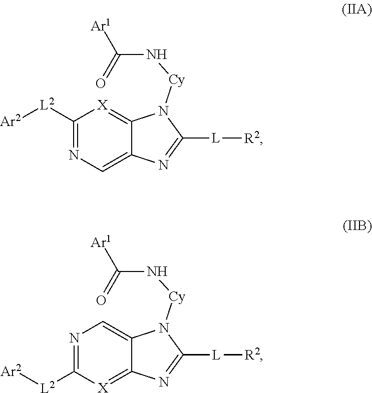

or a pharmaceutically acceptable salt thereof, wherein:
X is CH or N;
Cy is optionally substituted C₅-C₇ cycloalkyl or optionally substituted 6- or 7-membered heterocycloalkyl;
L is a bond, —O—, —NR^{a}—, or optionally substituted C₁-C₄alkylene;
L² is —O—, —NR^{a}—, or —CH₂—;
   each R^{a} is independently hydrogen or C₁-C₄alkyl;
R² is optionally substituted phenyl, optionally substituted pyridinyl, optionally substituted 5-membered heteroaryl, optionally substituted heterocycloalkyl, optionally substituted C₁-C₆alkyl, optionally substituted C₁-C₆alkoxy, optionally substituted C₁-C₆hydroxyalkyl, optionally substituted C₁-C₆haloalkyl, optionally substituted C₁-C₆haloalkoxy, optionally substituted C₃-C₆cycloalkyl, or NR^{f}R^{g};
   R^{f} and R^{g} are each independently hydrogen, C₁-C₆alkyl, C₁-C₆haloalkyl, OH, C₁-C₄alkoxy, C₁-C₆hydroxyalkyl, or C₃-C₆cycloalkyl;
Ar¹ is an optionally substituted five-membered heteroaryl; and
Ar² is optionally substituted heteroaryl, optionally substituted aryl, or optionally substituted 4-, 5- or 6-membered heterocycloalkyl.

In some aspects, the disclosure is directed to compounds of Formula IIIA, Formula IIIB, Formula IIIC, or Formula IIID:

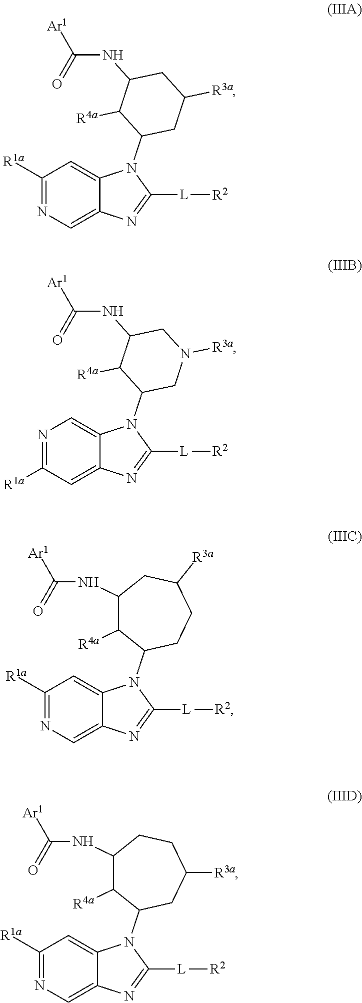

or a pharmaceutically acceptable salt thereof, wherein:
L is a bond, —O—, —NR^{a}—, or optionally substituted C₁-C₄alkylene;
   R^{a} is hydrogen or C₁-C₄alkyl;
R^{1a} is hydrogen, C₁-C₆alkyl, C₁-C₆alkoxy, halo, C₁-C₆haloalkyl, C₁-C₆haloalkoxy, CONR^{j}(C₁-C₄alkyl), CONR^{j}(C₃-C₄cycloalkyl), or NR^{b}R^{c};
   each R^{b} and R^{c} is independently hydrogen or C₁-C₄alkyl;
   each R^{j} is independently hydrogen or C₁-C₄alkyl;
R² is optionally substituted phenyl, optionally substituted pyridinyl, optionally substituted 5-membered heteroaryl, optionally substituted heterocycloalkyl, optionally substituted C₁-C₆alkyl, optionally substituted C₁-C₆alkoxy, optionally substituted C₁-C₆hydroxyalkyl, optionally substituted C₁-C₆haloalkyl, optionally substituted C₁-C₆haloalkoxy, optionally substituted C₃-C₆cycloalkyl, or NR^{f}R^{g};
   R^{f} and R^{g} are each independently hydrogen, C₁-C₆alkyl, C₁-C₆haloalkyl, OH, C₁-C₄alkoxy, C₁-C₆hydroxyalkyl, or C₃-C₆cycloalkyl;
one of R^{3a} or R^{4a} is hydrogen, deuterium, OH, cyano, C₁-C₃alkyl, C₁-C₃haloalkyl, C₁-C₃alkoxy, C₁-C₃haloalkoxy, deuterated C₁-C₃alkoxy, NH₂, NH(C₁-C₄alkyl), N(C₁-C₄alkyl)₂, OCO₂(C₁-C₄alkyl), OCO₂(C₃-C₄cycloalkyl), OSO₂(C₁-C₄alkyl), OSO₂(C₃-C₄cycloalkyl), OC(O)(C₁-C₄alkyl), OC(O)(C₃-C₄cycloalkyl), or NR^{m}Rⁿ; and the other of R^{3a} or R^{4a} is OH, cyano, C₁-C₃alkyl, C₁-C₃haloalkyl, C₁-C₃alkoxy, C₁-C₃haloalkoxy, deuterated C₁-C₃alkoxy, NH₂, NH(C₁-C₄alkyl), N(C₁-C₄alkyl)₂, OCO₂(C₁-C₄alkyl), OCO₂(C₃-C₄cycloalkyl), OSO₂(C₁-C₄alkyl), OSO₂(C₃-C₄cycloalkyl), OC(O)(C₁-C₄alkyl), OC(O)(C₃-C₄cycloalkyl), or NR^{m}Rⁿ; and wherein each R^{m} is independently hydrogen or C₁-C₄alkyl and each Rⁿ is independently CO₂(C₁-C₄alkyl), C(O)C₁-C₄alkyl, SO₂C₁-C₄alkyl, or SO₂(C₃-C₄cycloalkyl); and
Ar¹ is an optionally substituted five-membered heteroaryl.

In some aspects, the disclosure is directed to compounds of Formula IVA or Formula IVB:

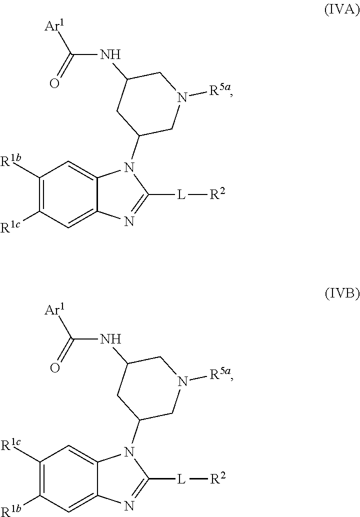

or a pharmaceutically acceptable salt thereof, wherein:
L is a bond, —O—, —NR^{a}—, or optionally substituted C₁-C₄alkylene;
   R^{a} is hydrogen or C₁-C₄alkyl;
R^{1b} is optionally substituted heteroaryl, optionally substituted aryl, optionally substituted 4-, 5- or 6-membered heterocycloalkyl, CONR^{j}(C₁-C₄alkyl), CONR^{j}(C₃-C₄cycloalkyl), or cyano;
   each R^{j} is hydrogen or C₁-C₄alkyl;
R^{1c} is hydrogen, halo, C₁-C₆alkyl, C₁-C₆haloalkyl, C₁-C₆alkoxy, or C₁-C₆haloalkoxy;
R² is optionally substituted phenyl, optionally substituted pyridinyl, optionally substituted 5-membered heteroaryl, optionally substituted heterocycloalkyl, optionally substituted C₁-C₆alkyl, optionally substituted C₁-C₆alkoxy, optionally substituted C₁-C₆hydroxyalkyl, optionally substituted C₁-C₆haloalkyl, optionally substituted C₁-C₆haloalkoxy, optionally substituted C₃-C₆cycloalkyl, or NR^{f}R^{g};
   R^{f} and R^{g} are each independently hydrogen, C₁-C₆alkyl, C₁-C₆haloalkyl, OH, C₁-C₄alkoxy, C₁-C₆hydroxyalkyl, or C₃-C₆cycloalkyl;
R^{5a} is hydrogen, C₁-C₆alkyl, C(O)C₁-C₆alkyl, C(O)NR^{b}R^{c}, oxo, OH, C₁-C₆alkoxy, C₁-C₆hydroxyalkyl, CO₂(C₁-C₆alkyl), SO₂(C₁-C₆alkyl), or SO₂(C₃-C₆cycloalkyl);
   each R^{b} and R^{c} is independently hydrogen or C₁-C₄alkyl; and
Ar¹ is an optionally substituted five-membered heteroaryl.

In some aspects, the disclosure is directed to compounds of Formula VA, Formula VB, Formula VC, or Formula VD:

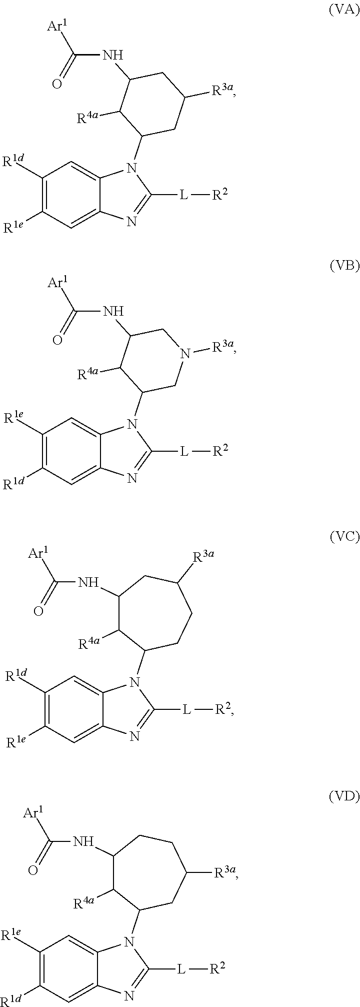

or a pharmaceutically acceptable salt thereof, wherein
L is a bond, —O—, —NR^{a}—, or optionally substituted C₁-C₄alkylene;
   R^{a} is hydrogen or C₁-C₄alkyl;
R^{1d} is optionally substituted heteroaryl, optionally substituted aryl, optionally substituted 4-, 5- or 6-membered heterocycloalkyl, CONR^{j}(C₁-C₄alkyl), CONR^{j}(C₃-C₄cycloalkyl), or cyano;
   each R^{j} is independently hydrogen or C₁-C₄alkyl;
R^{1e} is hydrogen, halo, C₁-C₆alkyl, C₁-C₆haloalkyl, C₁-C₆alkoxy, or C₁-C₆haloalkoxy;
R² is optionally substituted phenyl, optionally substituted pyridinyl, optionally substituted 5-membered heteroaryl, optionally substituted heterocycloalkyl, optionally substituted C₁-C₆alkyl, optionally substituted C₁-C₆alkoxy, optionally substituted C₁-C₆hydroxyalkyl, optionally substituted C₁-C₆haloalkyl, optionally substituted C₁-C₆haloalkoxy, optionally substituted C₃-C₆cycloalkyl, or NR^{f}R^{g};
   R^{f} and R^{g} are each independently hydrogen, C₁-C₆alkyl, C₁-C₆haloalkyl, OH, C₁-C₄alkoxy, C₁-C₆hydroxyalkyl, or C₃-C₆cycloalkyl one of R^{3a} or R^{4a} is hydrogen, deuterium, OH, cyano, C₁-C₃alkyl, C₁-C₃haloalkyl, C₁-C₃alkoxy, C₁-C₃haloalkoxy, deuterated C₁-C₃alkoxy, NH₂, NH(C₁-C₄alkyl), N(C₁-C₄alkyl)₂, OCO₂(C₁-C₄alkyl), OCO₂(C₃-C₄cycloalkyl), OSO₂(C₁-C₄alkyl), OSO₂(C₃-C₄cycloalkyl), OC(O)(C₁-C₄alkyl), OC(O)(C₃-C₄cycloalkyl), or NR^{m}Rⁿ; and the other of R^{3a} or R^{4a} is OH, cyano, C₁-C₃alkyl, C₁-C₃haloalkyl, C₁-C₃alkoxy, C₁-C₃haloalkoxy, deuterated C₁-C₃alkoxy, NH₂, NH(C₁-C₄alkyl), N(C₁-C₄alkyl)₂, OCO₂(C₁-C₄alkyl), OCO₂(C₃-C₄cycloalkyl), OSO₂(C₁-C₄alkyl), OSO₂(C₃-C₄cycloalkyl), OC(O)(C₁-C₄alkyl), OC(O)(C₃-C₄cycloalkyl), or NR^{m}Rⁿ; and wherein each R^{m} is independently hydrogen or C₁-C₄alkyl and each Rⁿ is independently CO₂(C₁-C₄alkyl), C(O)C₁-C₄alkyl, SO₂(C₁-C₄alkyl), or SO₂(C₃-C₄cycloalkyl); and
Ar¹ is an optionally substituted five-membered heteroaryl.

Stereoisomers and mixtures of stereoisomers of the compounds of Formulas IA, IB, IIA, IIB, IIIA, IIIB, IIIC, HID, IVA, IVB, VA, VB, VC, and VD, and the pharmaceutical salts thereof, are also described. Methods of using compounds of Formulas IA, IB, IIA, IIB, IIIA, IIIB, IIIC, IIID, IVA, IVB, VA, VB, VC, and VD are described, as well as pharmaceutical compositions including the compounds of Formulas IA, IB, IIA, IIB, IIIA, IIIB, IIIC, IIID, IVA, IVB, VA, VB, VC, and VD.

### BRIEF DESCRIPTION OF THE DRAWINGS

FIG. 1 illustrates the BCAA catabolism pathways, the point of BCAT2 involvement, and the points of catabolic impairment resulting in methylmalonic acidemia (MMA) and propionyl acidemia (PA).

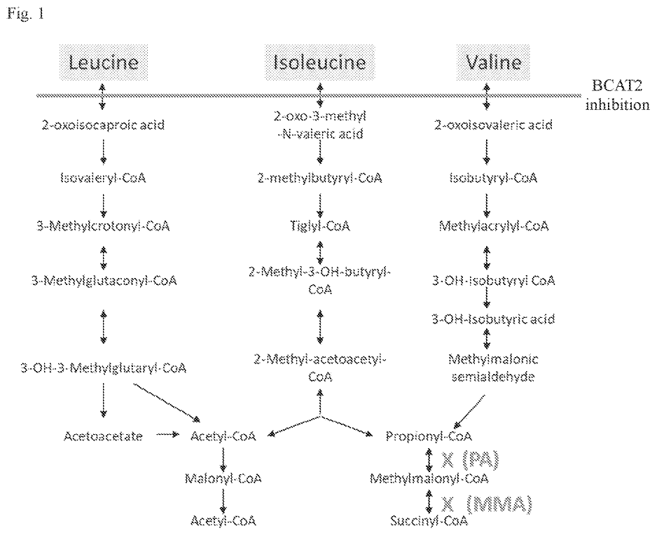

### DETAILED DESCRIPTION OF ILLUSTRATIVE EMBODIMENTS

Unless otherwise defined, all technical and scientific terms used herein have the same meaning as commonly understood by one of ordinary skill in the art to which this disclosure pertains. The terminology used in the description is for describing particular embodiments only and is not intended to be limiting of the disclosure.

The articles “a” and “an” as used herein and in the appended claims are used herein to refer to one or to more than one (e.g., to at least one) of the grammatical object of the article unless the context clearly indicates otherwise. By way of example, “an element” means one element or more than one element.

The term “compound”, as used herein, unless otherwise indicated, refers to any specific chemical compound disclosed herein and includes tautomers, optical isomers (enantiomers), other stereoisomers (diastereomers), and isotopologues thereof, as well as pharmaceutically acceptable salts and derivatives, including prodrugs and/or deuterated forms thereof. Deuterated forms of the compounds disclosed herein are those in which one or more of the hydrogen atoms of the compound have been replaced by deuterium. It is understood by those of ordinary skill that molecules which are described herein are stable compounds as generally described hereunder.

“Pharmaceutically acceptable” means approved or approvable by a regulatory agency of the Federal or a state government or the corresponding agency in a country other than the United States, or that is listed in the U.S. Pharmacopoeia or other generally recognized pharmacopoeia for use in animals, e.g., in humans.

“Pharmaceutically acceptable salt” refers to a salt of a compound of the disclosure that is pharmaceutically acceptable and that possesses the desired pharmacological activity of the parent compound. In particular, such salts are non-toxic and may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of a compound of the disclosure and that is compatible therewith.

The term “alkyl,” when used alone or as part of a substituent group, refers to a straight- or branched-chain hydrocarbon group having from 1 to 12 carbon atoms (“C₁-C₁₂”), for example 1 to 6 carbons atoms (“C₁-C₆”), in the group. Examples of alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), hexyl (C₆) (e.g., n-hexyl), heptyl (C₇) (e.g., n-heptyl), octyl (C₈) (e.g., n-octyl), and the like. In some embodiments, the alkyl group is a C₁-C₆alkyl; in other embodiments, it is a C₁-C₄alkyl; and in other embodiments, it is a C₁-C₃alkyl.

The term “alkylene,” when used alone or as part of a substituent group, refers to an alkyl diradical, i.e., a straight- or branched-chain hydrocarbon group that is attached to two other groups. For example, one embodiment of a C₂alkylene is the diradical —CH₂CH₂—. In some embodiments, the alkylene group is C₁-C₄alkylene.

When a range of carbon atoms is used herein, for example, C₁-C₆, all ranges, as well as individual numbers of carbon atoms are encompassed. For example, “C₁-C₃” includes C₁-C₃, C₁-C₂, C₂-C₃, C₁, C₂, and C₃.

The term “cycloalkyl” when used alone or as part of a substituent group refers to cyclic-containing, non-aromatic hydrocarbon groups having from 3 to 10 carbon atoms (“C₃-C₁₀”), for example from 3 to 7 carbon atoms (“C₃-C₇”). Examples of cycloalkyl groups include cyclopropyl (C₃), cyclobutyl (C₄), cyclopentyl (C₅), cyclohexyl (C₆), cycloheptyl (C₇), and the like. In some embodiments, the cycloalkyl group is a C₃-C₄cycloalkyl; in other embodiments, it is a C₃-C₆cycloalkyl; and in other embodiments, it is C₅-C₇cycloalkyl.

The term “heterocycloalkyl” when used alone or as part of a substituent group refers to any three to twelve-membered monocyclic, saturated or partially unsaturated ring containing at least one heteroatom that is oxygen, nitrogen, or sulfur. The heterocycloalkyl group may be attached to another group or substituent through any heteroatom or carbon atom of the ring that results in a stable structure. In some embodiments, heterocycloalkyl rings are characterized by the number of ring atoms in the heterocycloalkyl group. For example, a 6-membered heterocycloalkyl group refers to a heterocycloalkyl group having 6 ring atoms in the group. Similarly, a 5-membered heterocycloalkyl group refers to a heterocycloalkyl group having 5 ring atoms in the group. Similarly, a 4-membered heterocycloalkyl group refers to a heterocycloalkyl group having 4 ring atoms in the group. The heteroaryl moiety can be unsubstituted, or one or more of the carbon atoms, nitrogen, or sulfur atoms in the ring can be substituted. Examples of heterocycloalkyl groups include azepanyl, aziridinyl, azetidinyl, pyrrolidinyl, dioxolanyl, imidazolidinyl, pyrazolidinyl, piperazinyl, piperidinyl, dioxanyl, morpholinyl, thianyl, thianyl sulfoxide, 1-oxo-1-imino-1thiacyclohexyl, dithianyl, thiomorpholinyl, oxazepanyl, oxiranyl, oxetanyl, tetrahydrofuranyl, tetrahydropyranyl, tetrahydrothiophenyl, pyranyl, and the like. In some embodiments, the heterocycloalkyl is a three to twelve-membered monocyclic, saturated ring containing at least one heteroatom that is oxygen, nitrogen, or sulfur.

The terms “halo” or “halogen”, by itself or as part of another substituent, means a fluorine, chlorine, bromine, or iodine atom.

The term “aryl” when used alone or as part of a substituent group also refers to a mono- or bicyclic-aromatic hydrocarbon ring structure having 6 or 10 carbon atoms in the ring, wherein one or more of the carbon atoms in the ring is optionally substituted. The term “aryl” also includes a mono- or bicyclic-aromatic hydrocarbon ring structure having 6 or 10 carbon atoms in the ring, wherein two adjacent carbon atoms in the ring are optionally substituted such that said two adjacent carbon atoms and their respective substituents form a cycloalkyl or heterocycloalkyl ring. Examples of aryl groups include phenyl, indenyl, naphthyl, 1, 2, 3,4-tetrahydronaphthyl, and the like.

The term “heteroaryl” when used alone or as part of a substituent group refers to a mono- or bicyclic-aromatic ring structure including carbon atoms as well as up to four heteroatoms that are each independently nitrogen, oxygen, or sulfur. Heteroaryl rings can include a total of 5, 6, 9, or 10 ring atoms. In some embodiments, heteroaryl rings are characterized by the number of ring atoms in the heteroaryl group. For example, a 6-membered heteroaryl group refers to a heteroaryl group having 6 ring atoms in the group. Similarly, a 5-membered heteroaryl group refers to a heteroaryl group having 5 ring atoms in the group. The heteroaryl moiety can be unsubstituted, or one or more of the carbon atoms or nitrogen atoms in the ring can be substituted. Examples of heteroaryls include pyridyl, pyrrolyl, pyrazinyl, pyrimidinyl, pyridazinyl, pyrazolyl, thienyl, indolyl, isoindolyl, imidazolyl, oxazolyl, isoxazolyl, thiazolyl, furyl, oxadiazolyl, thiadiazolyl, quinolyl, isoquinolyl, benzothiazolyl, benzoisothiazolyl, benzoxazolyl, benzoisoxazolyl, benzofuranyl, isobenzofuranyl, benzothiophenyl, benzoimidazolyl, indazolyl, quinoxalyl, quinazolinyl, triazolyl, tetrazolyl, isothiazolyl, pyranyl, purinyl, naphthyridinyl, phthalazinyl, cinnolinyl, pteridinyl, 1H-pyrrolo[2,3-b]pyridinyl, oxazolo[4,5-b]pyridinyl, oxazolo[5,4-b]pyridinyl, thiazolo[5,4-b]pyridinyl, thiazolo[4,5-b]pyridinyl, 3H-imidazo[4,5-b]pyridinyl, furo[2,3-b]pyridinyl, thieno[2,3-b]pyridinyl, pyrazolo[1,5-a]pyridinyl, imidazol[1,5-a]pyridinyl, and pyrrolo[1,2]pyridazinyl and the like. The term “heteroaryl” also includes pyridine, pyrimidine, and pyrazines groups having a fused optionally substituted cycloalkyl or fused optionally substituted heterocycloalkyl group. A pyridine, pyrimidine, or pyrazine having a fused optionally substituted cycloalkyl or fused optionally substituted heterocycloalkyl group may be attached to the remainder of the molecule at any available carbon atom on the phenyl ring. Exemplary heteroaryls having a fused optionally substituted cycloalkyl or fused optionally substituted heterocycloalkyl group include but are not limited to:

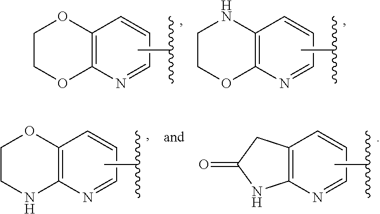

The term “optionally substituted,” as used herein to describe a chemical moiety defined herein, means that the moiety may, but is not required to be, substituted with one or more suitable functional groups or other substituents as provided herein. For example, a substituent may be optionally substituted with one or more of: halo (i.e., —F, —Cl, —Br, or —I), cyano, —NO₂, —N₃, —OH, —SH, C₁-C₆alkyl, C₃-C₆cycloalkyl, C₂-C₆alkenyl, C₂-C₆ alkynyl, C₁-C₆haloalkyl, C₁-C₆alkoxy, C₁-C₆ haloalkoxy, C₁-C₆alkylenethio, C₁-C₆alkyleneamino, —NH₂, —NH(C₁-C₆alkyl), —N(C₁-C₆alkyl)₂, —NH(C₁-C₆alkoxy), —C(O)NH₂, —C(O)NHC₁-C₆alkyl, —C(O)N(C₁-C₆alkyl)₂, —COOH, —C₁-C₆alkyleneCOOH, —C₃-C₆cycloalkylCOOH, —C₁-C₆alkyleneCONH₂, C₃-C₆cycloalkylCONH₂, —C₁-C₆alkyleneCONHC₁-C₆alkyl, —C₁-C₆alkyleneCON(C₁-C₆alkyl)₂, —C(O)C₁-C₆alkyl, —C(O)OC₁-C₆alkyl, —NHCO(C₁-C₆alkyl), —N(C₁-C₆alkyl)C(O)(C₁-C₆alkyl), —S(O)C₁-C₆alkyl, —S(O)C₃-C₆cycloalkyl, —S(O)₂C₁-C₆alkyl, —S(O)₂C₃-C₆cycloalkyl, oxo (═O), 3-7-membered heterocycloalkyl, aryl, or heteroaryl groups. In some embodiments, the C₁-C₆alkyl group in any of the substituent groups in this paragraph is a C₁-C₄alkyl; in other embodiments it is C₁-C₃alkyl. In some embodiments, the C₁-C₆alkylene group in any of the substituent groups in this paragraph is a C₁-C₄alkylene. In some embodiments, the C₁-C₆haloalkyl substituent is a C₁-C₄haloalkyl; in other embodiments, it is C₁-C₃haloalkyl. In some embodiments, the C₃-C₆cycloalkyl substituent is a C₃-C₄cycloalkyl substituent. In some embodiments, the C₁-C₆alkoxy substituent is a C₁-C₃alkoxy; in other embodiments, it is C₁-C₄alkoxy. In some embodiments, the C₁-C₆haloalkoxy substituent is a C₁-C₃haloalkoxy; in other embodiments, it is C₁-C₄haloalkoxy.

In other embodiments, “optionally substituted,” refers to the following substituents: halo, CN, NO₂, N₃, alkyl, alkenyl, alkynyl, cycloalkyl, aryl, heterocycloalkyl, heteroaryl, OR^{o1}, SR^{s1}, N(Rⁿ¹)₂, C(═O)N(Rⁿ¹)₂, N(Rⁿ¹)C(═O)R^{o1}, C(═O)R^{o1}, C(═O)OR^{o1}, OC(═O)R^{o1}, S(═O)R^{s1}, S(═O)₂R^{s1}, S(═O)(C₃-C₆cycloalkyl), S(═O)₂(C₃-C₆cycloalkyl), S(═O)OR^{o1}, OS(═O)R^{o1}, S(═O)₂OR^{o1}, OS(═O)₂R^{o1}, S(═O)N(Rⁿ¹)₂, S(═O)₂N(Rⁿ¹)₂, N(Rⁿ¹)S(═O)R^{s1}, N(Rⁿ¹)S(═O)₂R^{s1}, N(Rⁿ¹)C(═O)OR^{o1}, OC(═O)N(Rⁿ¹)₂, N(Rⁿ¹)C(═O)N(Rⁿ¹)₂, N(Rⁿ¹)S(═O)N(Rⁿ¹)₂, N(Rⁿ¹)S(═O)₂N(Rⁿ¹)₂, N(Rⁿ¹)S(═O)OR^{o1}, N(Rⁿ¹)S(═O)₂OR^{o1}, OS(═O)N(Rⁿ¹)₂, or OS(═O)₂N(Rⁿ¹)₂; wherein each instance of Rⁿ¹ is independently hydrogen, an optionally substituted C₁-C₆alkyl, or a nitrogen protecting group; each instance of R^{o1} is independently hydrogen, an optionally substituted C₁-C₆alkyl, or an oxygen protecting group; and each instance of R^{c1} is an optionally substituted C₁-C₆alkyl; and each instance of R^{s1} is independently an optionally substituted C₁-C₆alkyl or a sulfur protecting group.

As used herein, the term “alkenyl” refers to a straight- or branched-chain group having from 2 to 12 carbon atoms (“C₂-C₁₂”) in the group, wherein the group includes at least one carbon-carbon double bond. Examples of alkenyl groups include vinyl (—CH═CH₂; C₂alkenyl) allyl (—CH₂—CH═CH₂; C₃alkenyl), propenyl (—CH═CHCH₃; C₃alkenyl); isopropenyl (—C(CH₃)═CH₂; C₃alkenyl), butenyl (—CH═CHCH₂CH₃; C₄alkenyl), sec-butenyl (—C(CH₃)═CHCH₃; C₄alkenyl), iso-butenyl (—CH═C(CH₃)₂; C₄alkenyl), 2-butenyl (—CH₂CH═CHCH₃; C₄alkyl), pentenyl (—CH═CHCH₂CH₂CH₃; C₅alkenyl), and the like. In some embodiments, the alkenyl group is a C₂-C₄ alkenyl group.

As used herein, the term “alkynyl” refers to a straight- or branched-chain group having from 2 to 12 carbon atoms (“C₂-C₁₂”) in the group, and wherein the group includes at least one carbon-carbon triple bond. Examples of alkynyl groups include ethynyl (—C≡CH; C₂alkynyl); propargyl (—CH₂—C≡CH; C₃alkynyl), propynyl (—C≡CCH₃; C₃alkynyl); butynyl (—C≡CCH₂CH₃; C₄alkynyl), pentynyl (—C≡CCH₂CH₂CH₃; C₅alkynyl), and the like. In some embodiments, the alkynyl group is a C₂-C₄ alkynyl group.

As used herein, the term “alkoxy” refers to an oxygen radical attached to an alkyl group by a single bond. Examples of alkoxy groups include methoxy (—OCH₃), ethoxy (—OCH₂CH₃), propoxy (e.g., —OⁿPr, —OⁱPr), butoxy (e.g., —OⁿBu, —OⁱBu, —O^{s}Bu, —O^{t}Bu) and the like. In some embodiments, the alkoxy group is a C₁-C₆alkoxy; in other embodiments, it is a C₁-C₄alkoxy; and in other embodiments, it is a C₁-C₃alkoxy.

As used herein, the term “deuterated alkoxy” refers an alkoxy group as defined herein in which one or more hydrogen atoms has been replaced by deuterium. Examples of deuterated alkoxy groups include —OCD₃, —OCH₂CD₃, and the like. In some embodiments, the deuterated alkoxy group is a deuterated C₁-C₆alkoxy; in other embodiments, it is a deuterated C₁-C₄alkoxy; and in other embodiments, it is a deuterated C₁-C₃alkoxy.

As used herein, the term “alkoxy(alkylene)” as used by itself or as part of another group refers to an alkylene group as defined herein that is bound to an alkoxy group as defined herein. Examples of alkoxy(alkylene) groups include —CH₂OCH₃, —CH₂CH₂OCH₃, and the like.

As used herein, the term “haloalkyl” refers to an alkyl group wherein one or more of the hydrogen atoms has been replaced with one or more halogen atoms which may be the same or different. In some embodiments, the alkyl is substituted by at least one halogen. In other embodiments, the alkyl is substituted by one, two, or three F and/or Cl. Examples of haloalkyl groups include fluoromethyl (CH₂F), 1-fluoroethyl (CH(CH₃)F), 2-fluoroethyl, difluoromethyl (CHF₂), trifluoromethyl (CF₃), pentafluoroethyl, 1,1-difluoroethyl (C(CH₃)F₂), 2,2-difluoroethyl (CH₂CHF₂), 2,2,2-trifluoroethyl (CH₂CF₃), 2-fluoropropan-2-yl (C(CH₃)₂F), 3,3,3-trifluoropropyl, 4,4,4-trifluorobutyl, trichloromethyl and the like. In some embodiments, the haloalkyl group is a C₁-C₆haloalkyl; in other embodiments, it is a C₁-C₄haloalkyl; and in other embodiments, it is a C₁-C₃haloalkyl.

As used herein, the term “haloalkoxy” refers to an oxygen radical attached to a haloalkyl group by a single bond, wherein haloalkyl is defined above. Examples of haloalkoxy groups include fluoromethoxy (OCH₂F), 2-fluoroethoxy, difluoromethoxy (OCHF₂), trifluoromethoxy (OCF₃), pentafluoroethoxy, 1,1-difluoroethoxy (OC(CH₃)F₂), 2,2-difluoroethoxy, 2,2,2-trifluoroethoxy (OCH₂CF₃), 3,3,3-trifluoropropoxy, (1,1,1,3,3,3-hexafluoropropan-2-yl)oxy (OCH(CF₃)₂), 4,4,4-trifluorobutoxy, trichloromethoxy groups, and the like. In some embodiments, the haloalkoxy group is C₁-C₆haloalkoxy; in other embodiments, it is C₁-C₄haloalkoxy; and in other embodiments, it is C₁-C₃haloalkoxy.

As used herein, the term “hydroxyalkyl” refers to an alkyl group wherein one or more of the hydrogen atoms has been replaced with one or more hydroxyl (i.e., —OH) groups. In some embodiments, the hydroxyalkyl contains one OH. In other embodiments, the hydroxyalkyl contains two OH. In further embodiments, the hydroxyalkyl contains three OH. Examples of hydroxyalkyl groups include hydroxymethyl, hydroxyethyl (e.g., 1-hydroxyethyl, 2-hydroxyethyl), 1,2-dihydroxyethyl, hydroxypropyl (e.g., 2-hydroxypropyl, 3-hydroxypropyl), hydroxybutyl (e.g., 3-hydroxybutyl, 4-hydroxybutyl), 2-hydroxy-1-methylpropyl, 1,3 dihydroxyprop-2-yl, and the like. In some embodiments, the hydroxyalkyl group is C₁-C₆hydroxyalkyl; in other embodiments, it is C₁-C₄hydroxyalkyl; and in other embodiments, it is C₁-C₃hydroxyalkyl.

As used herein, the term “cyanoalkyl” as used by itself or as part of another group refers to an alkyl group wherein one or more of the hydrogen atoms has been replaced by one or cyano (i.e., —CN) groups. In some embodiments, the cyanoalkyl contains one cyano. In other embodiments, the cyanoalkyl contains two cyano. In further embodiments, the cyanoalkyl contains three cyano. Examples of cyanoalkyl groups include CH₂CN, CH₂CH₂CN, CH(CN)CH₃, CH₂CH₂CH₂CN, C(CH₃)₂CN, CH₂CH(CN)CH₃, CH(CN)CH₂CH₃, and the like. In some embodiments, the cyanoalkyl group is a C₁₋₆cyanoalkyl; in other embodiments, it is C₁₋₄cyanoalkyl; and in other embodiments, it is C₁₋₃cyanoalkyl. In yet other embodiments, the cyanoalkyl is a C₁₋₄cyanoalkyl.

As used herein, the term “imidazolidinonyl” refers to a five-membered saturated ring radical containing two-nitrogen atoms and three-carbon atoms wherein one of the carbon atoms is oxo-substituted (═O). Non-limiting examples include:

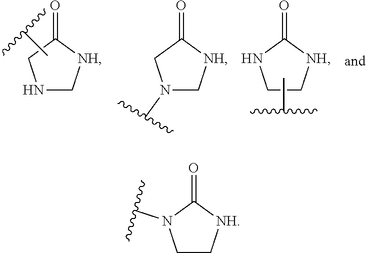

As used herein, the term “pyrrolidinonyl” refers to a five-membered saturated ring radical containing one nitrogen atom and four carbon atoms wherein one of the carbon atoms is oxo-substituted (═O). Non-limiting examples include:

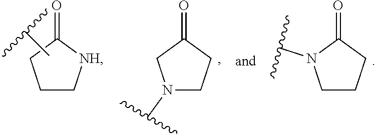

As used herein, the term “dihydropyrazolonyl” refers to a five-membered ring radical containing one unsaturation, two adjacent nitrogen atoms, and three carbon atoms wherein one of the carbon atoms is oxo-substituted (═O). Non-limiting examples include:

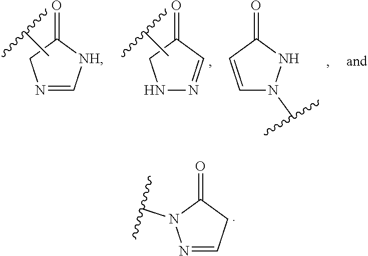

As used herein, the term “dihydropyrrolonyl” refers to a five-membered ring radical containing one unsaturation, one nitrogen atom, and four carbon atoms wherein one of the carbon atoms is oxo-substituted (═O). Non-limiting examples include:

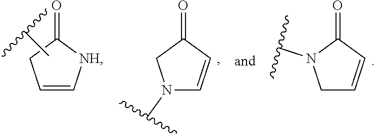

As used herein, the term “dihydroimidazolonyl” refers to a five-membered ring radical containing one unsaturation, two non-adjacent nitrogen atoms, and three carbon atoms wherein one of the carbon atoms is oxo-substituted (═O). Non-limiting examples include:

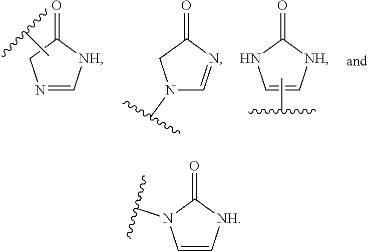

As used herein, the term “dihydrotriazolonyl” refers to a five-membered ring radical containing one unsaturation, three nitrogen atoms, and two carbon atoms wherein one of the carbon atoms is oxo-substituted (═O). Non-limiting examples include:

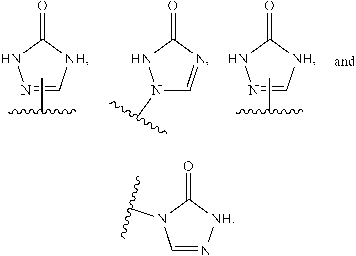

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including supercritical fluid chromatography (SFC), chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H. Tables of Resolving Agents and Optical Resolutions p. 268 (E. L. Eliel, Ed., Univ. of Notre Dame Press, Notre Dame, IN 1972).

Exemplary compounds of the disclosure including a chiral center may be depicted herein as having particular stereochemistries, but for which absolute stereochemistry has not been obtained. Absolute configurations can be obtained using methods known in the art.

In some embodiments, compounds of the disclosure are enantiomerically enriched, e.g., the enantiomeric excess or “ee” of the compound is greater than about 5% as measured by chiral HPLC. In another embodiment, the ee is greater than about 10%. In another embodiment, the ee is greater than about 20%. In another embodiment, the ee is greater than about 30%. In another embodiment, the ee is greater than about 40%. In another embodiment, the ee is greater than about 50%. In another embodiment, the ee is greater than about 60%. In another embodiment, the ee is greater than about 70%. In another embodiment, the ee is greater than about 80%. In another embodiment, the ee is greater than about 85%. In another embodiment, the ee is greater than about 90%. In another embodiment, the ee is greater than about 91%. In another embodiment, the ee is greater than about 92%. In another embodiment, the ee is greater than about 93%. In another embodiment, the ee is greater than about 94%. In another embodiment, the ee is greater than about 95%. In another embodiment, the ee is greater than about 96%. In another embodiment, the ee is greater than about 97%. In another embodiment, the ee is greater than about 98%. In another embodiment, the ee is greater than about 99%.

As used herein, the term “stereoisomers” refers to compounds which have identical chemical constitution and connectivity, but differ with regard to the arrangement of the atoms or groups in space, e.g., enantiomers or diastereomers.

The term “nitrogen protecting group” refers to a moiety that is attached to a nitrogen atom to prevent reaction at that nitrogen atom. Nitrogen protecting groups will be known by those skilled in the art and include those described in Wuts, P. G., Greene's Protective Groups in Organic Synthesis. Wiley; 5th edition (Oct. 27, 2014), which is incorporated by reference herein.

The term “oxygen protecting group” refers to a moiety that is attached to an oxygen atom to prevent reaction at that oxygen atom. Oxygen protecting groups will be known by those skilled in the art and include those described in Wuts, P. G., Greene's Protective Groups in Organic Synthesis. Wiley; 5th edition (Oct. 27, 2014), which is incorporated by reference herein.

The term “sulfur protecting group” refers to a moiety that is attached to a sulfur atom to prevent reaction at that sulfur atom. Sulfur protecting groups will be known by those skilled in the art and include those described in Wuts, P. G., Greene's Protective Groups in Organic Synthesis. Wiley; 5th edition (Oct. 27, 2014), which is incorporated by reference herein.

As used in structures herein,

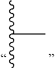

indicates the point of attachment of the particular depicted structure or substituent group to the appropriate atom(s) in the remainder of the molecule.

It will be understood that some embodiments of the compounds disclosed herein may exist in multiple tautomeric forms. Such embodiments are to be understood as encompassing all possible tautomers.

As used herein, the term “isotopologue” is an isotopically enriched compound. As used herein, and unless otherwise indicated, the term “isotopically enriched” refers to an atom having an isotopic composition other than the naturally abundant isotopic composition of that atom. “Isotopically enriched” may also refer to a compound containing at least one atom having an isotopic composition other than the natural isotopic composition of that atom. In an isotopologue, “isotopic enrichment” refers to the percentage of incorporation of an amount of a specific isotope of a given atom in a molecule in the place of that atom's natural isotopic composition. For example, deuterium enrichment of 1% at a given position means that 1% of the molecules in a given sample contain deuterium at the specified position. Because the naturally occurring distribution of deuterium is about 0.0156%, deuterium enrichment at any position in a compound synthesized using non-enriched starting materials is about 0.0156%.

Thus, as used herein, and unless otherwise indicated, the term “isotopic enrichment factor” refers to the ratio of the isotopic composition to the natural isotopic composition of a specified isotope. For example, a deuterium isotopic enrichment of 1000 at a particular position means that about 15% of the hydrogen atoms at that position are deuterium (15/0.015=1000).

With regard to the compounds provided herein, when a particular atom's position is designated as having deuterium or “D” or “²H”, it is understood that the abundance of deuterium at that position is substantially greater than the natural abundance of deuterium, which is about 0.015%. A position designated as having deuterium typically has a minimum isotopic enrichment factor of, in particular embodiments, at least 1000 (15% deuterium incorporation), at least 2000 (30% deuterium incorporation), at least 3000 (45% deuterium incorporation), at least 3500 (52.5% deuterium incorporation), at least 4000 (60% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation) at each designated deuterium atom. The isotopic enrichment and isotopic enrichment factor of the compounds provided herein can be determined using conventional analytical methods known to one of ordinary skill in the art, including mass spectrometry and nuclear magnetic resonance spectroscopy.

The term “patient” or “subject” is used throughout the specification to describe an animal, preferably a human or a domesticated animal, to whom treatment, including prophylactic treatment, with the compounds or compositions according to the present disclosure is provided. For treatment of those conditions or disease states which are specific for a specific animal such as a human patient, the term patient refers to that specific animal, including a domesticated animal such as a dog or cat or a farm animal such as a horse, cow, sheep, etc. In general, in the present disclosure, the term patient refers to a human patient unless otherwise stated or implied from the context of the use of the term.

In some embodiments, a subject is treated with compounds of the disclosure, or a pharmaceutical composition comprising compounds of the disclosure. In certain aspects, the subject is a human subject, such as is a human adult over 18 years old in need of treatment. In further embodiments, the human subject is a human child less than 18 years old in need of treatment.

The terms “therapeutically effective amount” or “effective amount” means an amount or dose of a compound of the disclosure (or a pharmaceutically acceptable salt thereof) sufficient to generally bring about the desired therapeutic benefit in subjects in need of such treatment for the designated disease or disorder. Further, a therapeutically effective amount with respect to a compound of the disclosure means that amount of therapeutic agent alone, or in combination with other therapies, that provides a therapeutic benefit in the treatment or prevention of a disease.

“Treating” or “treatment” of any disease or disorder refers, in one embodiment, to ameliorating the disease or disorder (e.g., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

The terms “prevent,” “preventing,” and “prevention” refer to the prevention of the onset, recurrence, or spread of the disease in a subject resulting from the administration of a prophylactic or therapeutic agent.

In some aspects, the disclosure is directed to compounds of Formula IA or Formula IB:

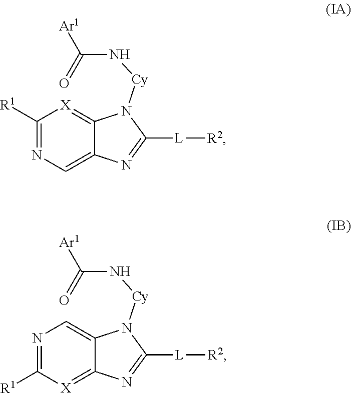

or a pharmaceutically acceptable salt thereof, wherein Ar¹, Cy, L, R¹, R², and X are defined herein.

In some embodiments, the compound is a compound of Formula IA or a pharmaceutically acceptable salt thereof,

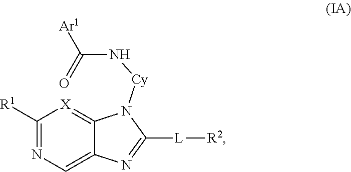

wherein Ar¹, Cy, L, R¹, R², and X are defined herein.

In some embodiments, the compound is a compound of Formula IA.

In other embodiments, the compound is a pharmaceutically acceptable salt of a compound of Formula IA.

In some embodiments, the compound is a compound of Formula IB or a pharmaceutically acceptable salt thereof,

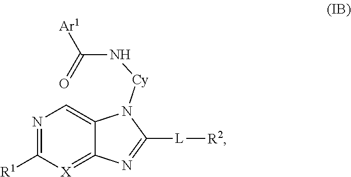

wherein Ar¹, Cy, L, R¹, R², and X are defined herein.

In some embodiments, the compound is a compound of Formula IB.

In other embodiments, the compound is a pharmaceutically acceptable salt of a compound of Formula IB.

In some aspects, the disclosure is directed to compounds of Formula IIA or Formula IIB:

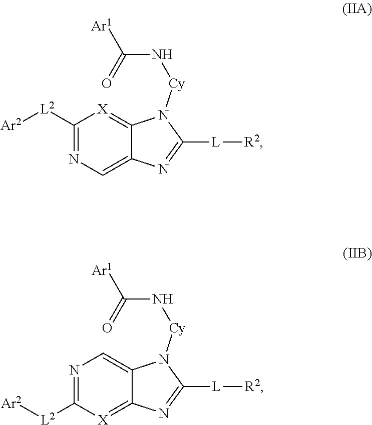

or a pharmaceutically acceptable salt thereof, wherein Ar¹, Ar², Cy, L, L², R², and X are defined herein.

In some embodiments, the compound is a compound of Formula IIA or a pharmaceutically acceptable salt thereof,

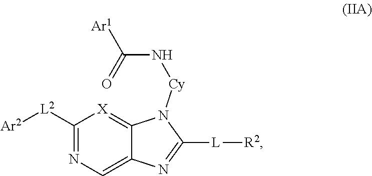

wherein Ar¹, Ar², Cy, L, L², R², and X are defined herein.

In some embodiments, the compound is a compound of Formula IIA.

In other embodiments, the compound is a pharmaceutically acceptable salt of a compound of Formula IIA.

In some embodiments, the compound is a compound of Formula IIB or a pharmaceutically acceptable salt thereof,

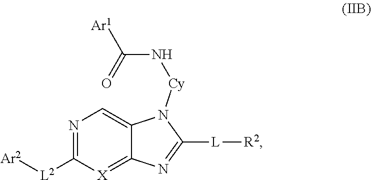

wherein Ar¹, Ar², Cy, L, L², R², and X are defined herein.

In some embodiments, the compound is a compound of Formula IIB.

In other embodiments, the compound is a pharmaceutically acceptable salt of a compound of Formula IIB.

In some aspects, the disclosure is directed to compounds of Formula IIIA, Formula IIIB, Formula IIIC, or Formula IIID:

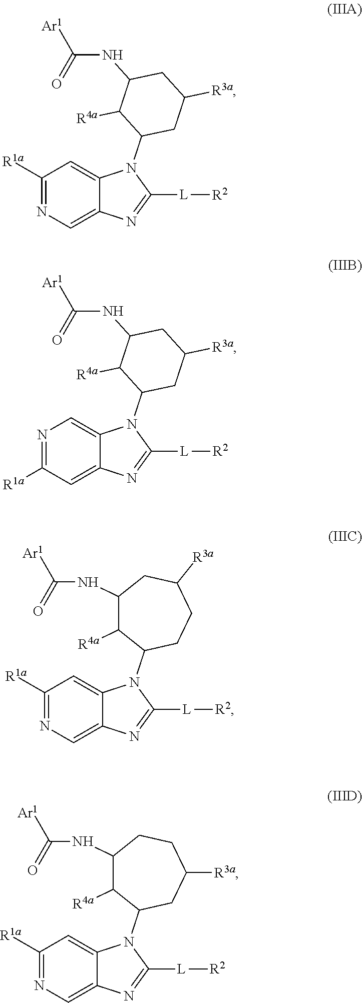

or a pharmaceutically acceptable salt thereof, wherein Ar¹, L, R^{1a}, R², R^{3a}, and R^{4a} are defined herein.

In some embodiments, the compound is a compound of Formula IIIA or a pharmaceutically acceptable salt thereof,

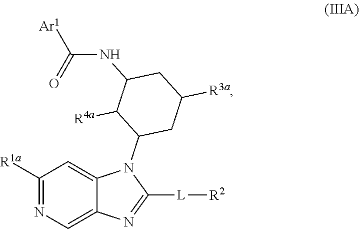

wherein Ar¹, L, R^{1a}, R², R^{3a}, and R^{4a} are defined herein.

In some embodiments, the compound is a compound of Formula IIIA.

In other embodiments, the compound is a pharmaceutically acceptable salt of a compound of Formula IIIA.

In some embodiments, the compound is a compound of Formula IIIB or a pharmaceutically acceptable salt thereof,

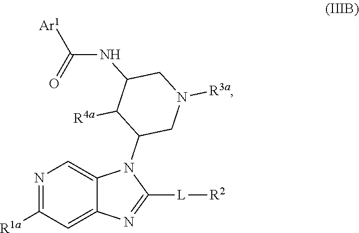

wherein Ar¹, L, R^{1a}, R², R^{3a}, and R^{4a} are defined herein.

In some embodiments, the compound is a compound of Formula IIIB.

In other embodiments, the compound is a pharmaceutically acceptable salt of a compound of Formula IIIB.

In some embodiments, the compound is a compound of Formula IIIC or a pharmaceutically acceptable salt thereof,

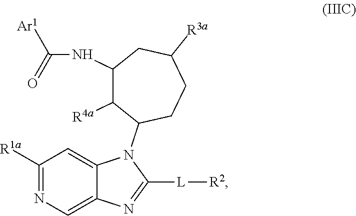

wherein Ar¹, L, R^{1a}, R², R^{3a}, and R^{4a} are defined herein.

In some embodiments, the compound is a compound of Formula IIIC.

In other embodiments, the compound is a pharmaceutically acceptable salt of a compound of Formula IIIC.

In some embodiments, the compound is a compound of Formula IIID or a pharmaceutically acceptable salt thereof,

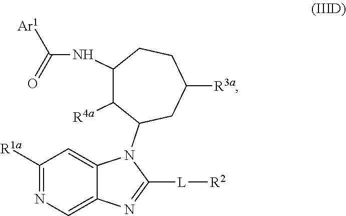

wherein Ar¹, L, R^{1a}, R², R^{3a}, and R^{4a} are defined herein.

In some embodiments, the compound is a compound of Formula IIID.

In other embodiments, the compound is a pharmaceutically acceptable salt of a compound of Formula IIID.

In some aspects, the disclosure is directed to compounds of Formula IVA or Formula IVB:

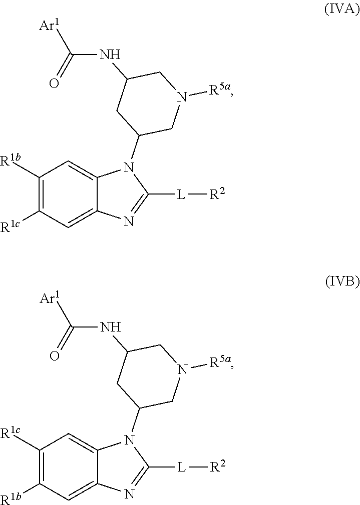

or a pharmaceutically acceptable salt thereof, wherein Ar¹, L, R^{1b}, R^{1c}, R², and R^{5a} are defined herein.

In some embodiments, the compound is a compound of Formula IVA or a pharmaceutically acceptable salt thereof,

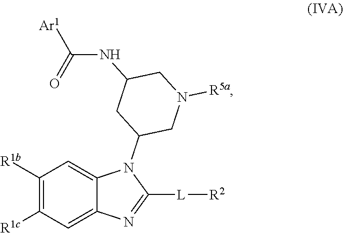

wherein Ar¹, L, R^{1b}, R^{1c}, R², and R^{5a} are defined herein.

In some embodiments, the compound is a compound of Formula IVA.

In other embodiments, the compound is a pharmaceutically acceptable salt of a compound of Formula IVA.

In some embodiments, the compound is a compound of Formula IVB or a pharmaceutically acceptable salt thereof,

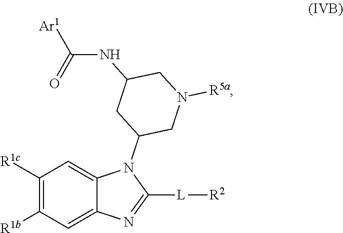

wherein Ar¹, L, R^{1b}, R^{1c}, R², and R^{5a} are defined herein.

In some embodiments, the compound is a compound of Formula IVB.

In other embodiments, the compound is a pharmaceutically acceptable salt of a compound of Formula IVB.

In some aspects, the disclosure is directed to compounds of Formula VA, Formula VB, Formula VC, or Formula VD:

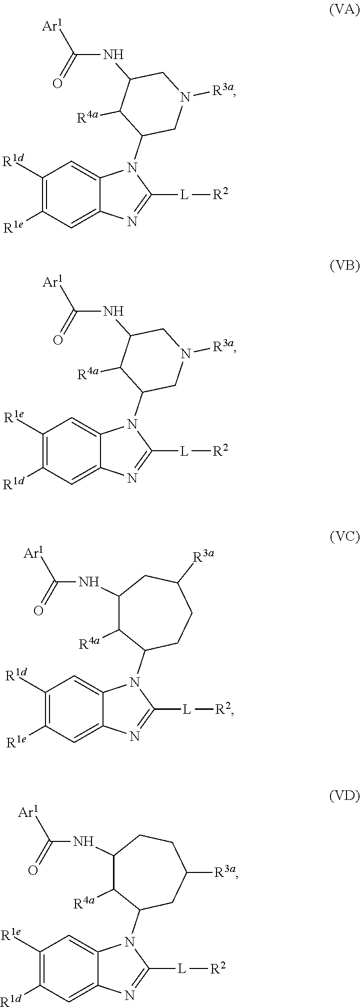

or a pharmaceutically acceptable salt thereof, wherein Ar¹, L, R^{1d}, R^{1e}, R², R^{3a}, and
R^{4a} are defined herein.

In some embodiments, the compound is a compound of Formula VA or a pharmaceutically acceptable salt thereof,

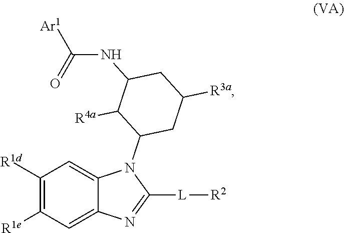

wherein Ar¹, L, R^{1d}, R^{1e}, R², R^{3a}, and R^{4a} are defined herein.

In some embodiments, the compound is a compound of Formula VA.

In other embodiments, the compound is a pharmaceutically acceptable salt of a compound of Formula VA.

In some embodiments, the compound is a compound of Formula VB or a pharmaceutically acceptable salt thereof,

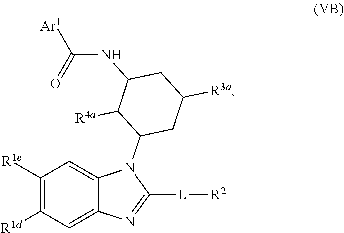

wherein Ar¹, L, R^{1d}, R^{1e}, R², R^{3a}, and R^{4a} are defined herein.

In some embodiments, the compound is a compound of Formula VB.

In other embodiments, the compound is a pharmaceutically acceptable salt of a compound of Formula VB.

In some embodiments, the compound is a compound of Formula VC or a pharmaceutically acceptable salt thereof,

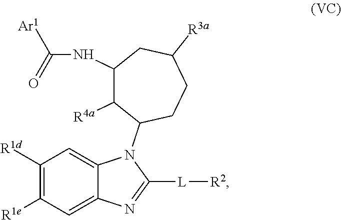

wherein Ar¹, L, R^{1d}, R^{1e}, R², R^{3a}, and R^{4a} are defined herein.

In some embodiments, the compound is a compound of Formula VC.

In other embodiments, the compound is a pharmaceutically acceptable salt of a compound of Formula VC.

In some embodiments, the compound is a compound of Formula VD or a pharmaceutically acceptable salt thereof,

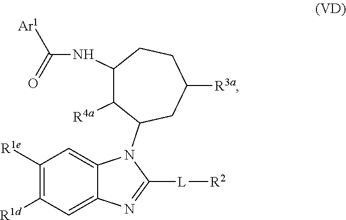

wherein Ar¹, L, R^{1d}, R^{1e}, R², R^{3a}, and R^{4a} are defined herein.

In some embodiments, the compound is a compound of Formula VD.

In other embodiments, the compound is a pharmaceutically acceptable salt of a compound of Formula VD.

In some aspects, X in the compounds of Formula IA, Formula IB, Formula IIA, or Formula IIB is CH or N.

In some embodiments, X in the compounds of Formula IA, Formula IB, Formula IIA, or Formula IIB is CH.

In other embodiments, X in the compounds of Formula IA, Formula IB, Formula IIA, or Formula IIB is N.

In some aspects, R¹ in the compounds of Formula IA or compounds of Formula IB is an optionally substituted heteroaryl, optionally substituted aryl, or optionally substituted 4-, 5-, or 6-membered heterocycloalkyl.

In some embodiments, R¹ in the compounds of Formula IA or compounds of Formula IB is an optionally substituted heteroaryl.

In some embodiments, R¹ in the compounds of Formula IA or compounds of Formula IB is an optionally substituted 5- or 6-membered heteroaryl, optionally substituted imidazolidinonyl, optionally substituted pyrrolidinonyl, optionally substituted dihydropyrazolonyl, optionally substituted dihydropyrrolonyl, optionally substituted dihydroimidazolonyl, or optionally substituted dihydrotriazolonyl.

In some embodiments, R¹ in the compounds of Formula IA or compounds of Formula IB is an optionally substituted 5- or 6-membered heteroaryl, such as, for example, optionally substituted: pyrrolyl, pyrazolyl, imidazolyl, triazolyl, tetrazolyl, oxadiazolyl, oxazolyl, isoxazolyl, isothiazolyl, thiazolyl, thiadiazolyl, pyridinyl, pyrimidinyl, pyrazinyl, or pyridazinyl.

In some embodiments, R¹ in the compounds of Formula IA or compounds of Formula IB is an unsubstituted 5- or 6-membered heteroaryl, such as, for example, an unsubstituted: pyrrolyl, pyrazolyl, imidazolyl, triazolyl, tetrazolyl, oxadiazolyl, oxazolyl, isoxazolyl, isothiazolyl, thiazolyl, thiadiazolyl, pyridinyl, pyrimidinyl, pyrazinyl, or pyridazinyl.

In other embodiments, R¹ in the compounds of Formula IA or compounds of Formula IB is a substituted 5- or 6-membered heteroaryl, such as, for example, a substituted: pyrrolyl, pyrazolyl, imidazolyl, triazolyl, tetrazolyl, oxadiazolyl, oxazolyl, isoxazolyl, isothiazolyl, thiazolyl, thiadiazolyl, pyridinyl, pyrimidinyl, pyrazinyl, or pyridazinyl. In some embodiments, the 5- or 6-membered heteroaryl is substituted with one or more of NR^{b}R^{c}(wherein each R^{b} and R^{c} is independently hydrogen or C₁-C₄alkyl), oxo, cyano, C₁-C₃alkyl, C₁-C₃haloalkyl, C₁-C₆hydroxyalkyl, or —C(O)OC₁-C₃alkyl. In further embodiments, the 5- or 6-membered heteroaryl is substituted with one or more of NR^{b}R^{c} (wherein each R^{b} and R^{c} is independently hydrogen or C₁-C₄alkyl), halo, oxo, cyano, C₁-C₄alkyl, C₁-C₄haloalkyl, C₁-C₄alkoxy, C₁-C₆hydroxyalkyl, or —C(O)OC₁-C₄alkyl. In yet other embodiments, the 5- or 6-membered heteroaryl is substituted with one or more of oxo, NR^{b}R^{c}(wherein each R^{b} and R^{c} is independently hydrogen or C₁-C₄alkyl), C₁-C₄alkyl, C₁-C₄haloalkyl, C₁-C₆hydroxyalkyl, halo, cyano, C₁-C₄alkoxy, C₁-C₄haloalkoxy or CO₂(C₁-C₄alkyl).

In some embodiments, R¹ in the compounds of Formula IA or compounds of Formula IB is an optionally substituted 5-membered heteroaryl, such as, for example, optionally substituted: pyrrolyl, pyrazolyl, imidazolyl, triazolyl, tetrazolyl, oxadiazolyl, oxazolyl, isoxazolyl, isothiazolyl, thiazolyl, or thiadiazolyl.

In other embodiments, R¹ in the compounds of Formula IA or compounds of Formula IB is an optionally substituted: oxadiazolyl, thiadiazolyl, oxazolyl, pyrazolyl, imidazolyl, triazolyl, tetrazolyl, or thiazolyl.

In some embodiments, R¹ in the compounds of Formula IA or compounds of Formula IB is a substituted 5-membered heteroaryl, such as, for example, a substituted: pyrrolyl, pyrazolyl, imidazolyl, triazolyl, tetrazolyl, oxadiazolyl, oxazolyl, isoxazolyl, isothiazolyl, thiazolyl, or thiadiazolyl. In further embodiments, the 5-membered heteroaryl is substituted with one or more of oxo, NR^{b}R^{c}, C₁-C₄alkyl, C₁-C₄haloalkyl, C₁-C₆hydroxyalkyl, halo, or C(O)OC₁-C₄alkyl; wherein each R^{b} and R^{c} is independently hydrogen or C₁-C₄alkyl. In yet other embodiments, the 5-membered heteroaryl is substituted with one or more of oxo, NR^{b}R^{c}(wherein each R^{b} and R^{c} is independently hydrogen or C₁-C₄alkyl), C₁-C₄alkyl, C₁-C₄haloalkyl, C₁-C₆hydroxyalkyl, halo, cyano, C₁-C₄alkoxy, C₁-C₄haloalkoxy or CO₂(C₁-C₄alkyl).

In other embodiments, R¹ in the compounds of Formula IA or compounds of Formula IB is an unsubstituted 5-membered heteroaryl, such as, for example, an unsubstituted: pyrrolyl, pyrazolyl, imidazolyl, triazolyl, tetrazolyl, oxadiazolyl, oxazolyl, isoxazolyl, isothiazolyl, thiazolyl, or thiadiazolyl.

In some embodiments, R¹ in the compounds of Formula IA or compounds of Formula IB is

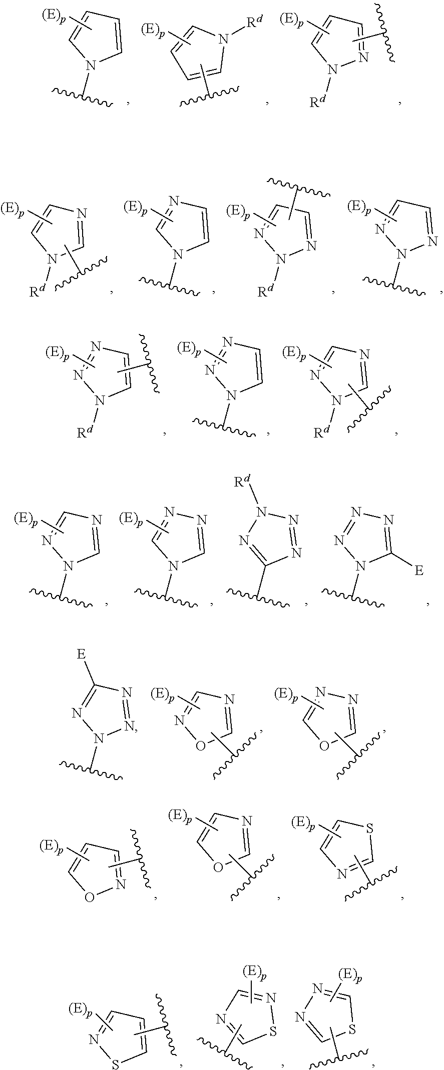

wherein:
each instance of R^{d} is independently hydrogen, optionally substituted C₁-C₆alkyl, or a nitrogen protecting group;
each instance of E is independently halo, CN, NO₂, N₃, an optionally substituted alkyl, an optionally substituted alkenyl, an optionally substituted alkynyl, an optionally substituted cycloalkyl, an optionally substituted aryl, an optionally substituted heterocycloalkyl, an optionally substituted heteroaryl, OR^{o1}, SR^{s1}, N(Rⁿ¹)₂, C(═O)N(Rⁿ¹)₂, N(Rⁿ¹)C(═O)R^{o1}, C(═O)R^{o1}, C(═O)OR^{o1}, OC(═O)R^{o1}, S(═O)R^{s1}, S(═O)₂R^{s1}, S(═O)OR^{o1}, OS(═O)R^{o1}, S(═O)₂OR^{o1}, OS(═O)₂R^{o1}, S(═O)N(Rⁿ¹)₂, S(═O)₂N(Rⁿ¹)₂, S(═O)(C₃-C₆cycloalkyl), S(═O)₂(C₃-C₆cycloalkyl), N(Rⁿ¹)S(═O)R^{s1}, N(Rⁿ¹)S(═O)₂R^{s1}, N(Rⁿ¹)C(═O)OR^{o1}, OC(═O)N(Rⁿ¹)₂, N(Rⁿ¹)C(═O)N(Rⁿ¹)₂, N(Rⁿ¹)S(═O)N(Rⁿ¹)₂, N(Rⁿ¹)S(═O)₂N(Rⁿ¹)₂, N(Rⁿ¹)S(═O)OR^{o1}, N(Rⁿ¹)S(═O)₂OR^{o1}, OS(═O)N(Rⁿ¹)₂, or OS(═O)₂N(Rⁿ¹)₂; or alternatively two instances of (E)ₚ attached to adjacent carbon atoms, can be taken together with the carbon atom(s) to which they are attached to form an optionally substituted cycloalkyl or a heterocycloalkyl; wherein:
   each instance of Rⁿ¹ is independently hydrogen, an optionally substituted C₁-C₆alkyl, or a nitrogen protecting group;
   each instance of R^{o1} is independently hydrogen, an optionally substituted C₁-C₆alkyl, or an oxygen protecting group; and
   each instance of R^{c1} is independently an optionally substituted C₁-C₆alkyl; and
   each instance of R^{s1} is independently an optionally substituted C₁-C₆alkyl or a sulfur protecting group; and
p is 0, 1, 2, 3, or 4, as valency permits. Where a particular atom is not substituted with E, it should be understood as having the required number of hydrogens as valency permits so as to form a stable ring.

In some embodiments, R¹ in the compounds of Formula IA or compounds of Formula IB is

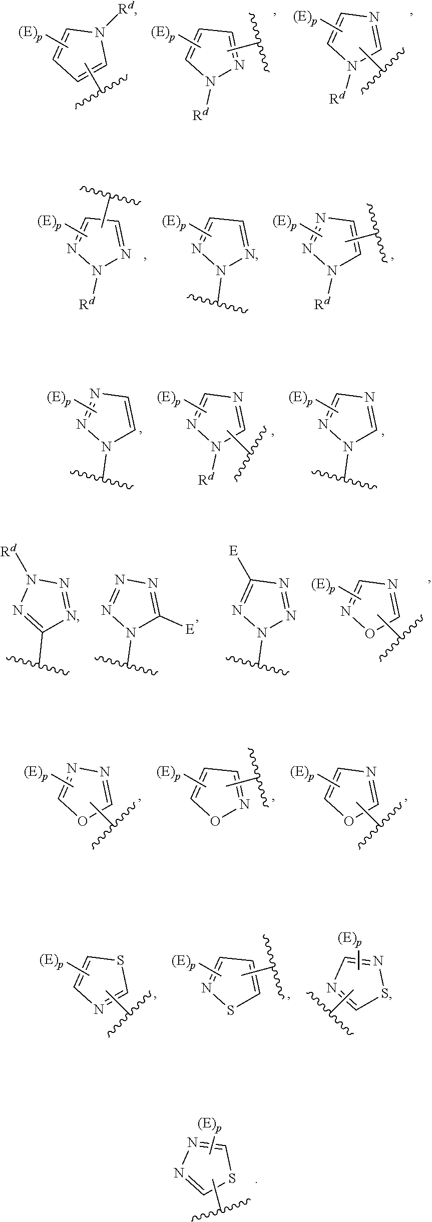

In some embodiments, R¹ in the compounds of Formula IA or compounds of Formula IB is unsubstituted: oxadiazolyl, oxazolyl, pyrazolyl, triazolyl, or tetrazolyl.

In some embodiments, R¹ in the compounds of Formula IA or compounds of Formula IB is unsubstituted: oxadiazolyl, oxazolyl, pyrazolyl, triazolyl, tetrazolyl, or thiazolyl.

In some embodiments, R¹ in the compounds of Formula IA or compounds of Formula IB is unsubstituted oxadiazolyl.

In some embodiments, R¹ in the compounds of Formula IA or compounds of Formula IB is unsubstituted 1,3,4-oxadiazol-2-yl. In other embodiments, R¹ in the compounds of Formula IA or compounds of Formula IB is unsubstituted 1,2,4-oxadiazol-3-yl.

In some embodiments, R¹ in the compounds of Formula IA or compounds of Formula IB is unsubstituted oxazolyl.

In some embodiments, R¹ in the compounds of Formula IA or compounds of Formula IB is unsubstituted oxazol-5-yl. In other embodiments, R¹ in the compounds of Formula IA or compounds of Formula IB is unsubstituted oxazol-2-yl.

In some embodiments, R¹ in the compounds of Formula IA or compounds of Formula IB is unsubstituted triazolyl.

In some embodiments, R¹ in the compounds of Formula IA or compounds of Formula IB is unsubstituted 1,2,3-triazol-2-yl. In other embodiments, R¹ in the compounds of Formula IA or compounds of Formula IB is unsubstituted 1,2,4-triazol-1-yl.

In further embodiments, R¹ in the compounds of Formula IA or compounds of Formula IB is unsubstituted 1,2,3-triazol-1-yl. In other embodiments, R¹ in the compounds of Formula IA or compounds of Formula IB is unsubstituted 1,2,4-triazol-3-yl. In further embodiments, R¹ in the compounds of Formula IA or compounds of Formula IB is unsubstituted 1,2,3-triazol-5-yl. In other embodiments, R¹ in the compounds of Formula IA or compounds of Formula IB is unsubstituted 1,2,4-triazol-5-yl.

In some embodiments, R¹ in the compounds of Formula IA or compounds of Formula IB is unsubstituted pyrazolyl.

In some embodiments, R¹ in the compounds of Formula IA or compounds of Formula IB is unsubstituted pyrazol-1-yl. In other embodiments, R¹ in the compounds of Formula IA or compounds of Formula IB is unsubstituted pyrazol-3-yl. In further embodiments, R¹ in the compounds of Formula IA or compounds of Formula IB is unsubstituted pyrazol-4-yl. In other embodiments, R¹ in the compounds of Formula IA or compounds of Formula IB is unsubstituted pyrazol-5-yl.

In some embodiments, R¹ in the compounds of Formula IA or compounds of Formula IB is unsubstituted tetrazolyl.

In some embodiments, R¹ in the compounds of Formula IA or compounds of Formula IB is unsubstituted tetrazol-5-yl.

In some embodiments, R¹ in the compounds of Formula IA or compounds of Formula IB is unsubstituted thiazolyl.

In some embodiments, R¹ in the compounds of Formula IA or compounds of Formula IB is unsubstituted thiazol-5-yl. In other embodiments, R¹ in the compounds of Formula IA or compounds of Formula IB is unsubstituted thiazol-4-yl. In further embodiments, R¹ in the compounds of Formula IA or compounds of Formula IB is unsubstituted thiazol-2-yl.

In some embodiments, R¹ in the compounds of Formula IA or compounds of Formula IB is unsubstituted thiadiazolyl.

In some embodiments, R¹ in the compounds of Formula IA or compounds of Formula IB is unsubstituted 1,3,4-thiadiazol-2-yl. In other embodiments, R¹ in the compounds of Formula IA or compounds of Formula IB is unsubstituted 1,2,4-thiadiazol-5-yl.

In some embodiments, R¹ in the compounds of Formula IA or compounds of Formula IB is unsubstituted imidazolyl.

In some embodiments, R¹ in the compounds of Formula IA or compounds of Formula IB is unsubstituted imidazol-2-yl.

In some embodiments, R¹ in the compounds of Formula IA or compounds of Formula IB is substituted oxazolyl.

In some embodiments, R¹ in the compounds of Formula IA or compounds of Formula IB is oxazolyl substituted with a C₁-C₄alkyl group. In other embodiments, R¹ in the compounds of Formula IA or compounds of Formula IB is oxazolyl substituted with a C₁-C₃alkyl group. In further embodiments, R¹ in the compounds of Formula IA or compounds of Formula IB is oxazolyl substituted with a methyl group.

In some embodiments, R¹ in the compounds of Formula IA or compounds of Formula IB is 5-methyloxazol-2-yl. In other embodiments, R¹ in the compounds of Formula IA or compounds of Formula IB is 4-methyloxazol-2-yl.

In some embodiments, R¹ in the compounds of Formula IA or compounds of Formula IB is substituted thiazolyl.

In some embodiments, R¹ in the compounds of Formula IA or compounds of Formula IB is thiazolyl substituted with one or more C₁-C₄alkyl, C₁-C₆hydroxyalkyl, halo, or NR^{b}R^{c}, wherein each R^{b} and R^{c} is independently hydrogen or C₁-C₄alkyl.

In some embodiments, R¹ in the compounds of Formula IA or compounds of Formula IB is thiazolyl substituted with a C₁-C₄alkyl group. In other embodiments, R¹ in the compounds of Formula IA or compounds of Formula IB is thiazolyl substituted with a C₁-C₃alkyl group. In further embodiments, R¹ in the compounds of Formula IA or compounds of Formula IB is thiazolyl substituted with a methyl group.

In some embodiments, R¹ in the compounds of Formula IA or compounds of Formula IB is 5-methylthiazol-2-yl.

In some embodiments, R¹ in the compounds of Formula IA or compounds of Formula IB is substituted triazolyl.

In other embodiments, R¹ in the compounds of Formula IA or compounds of Formula IB is triazolyl substituted with one or more C₁-C₄alkyl group, C₁-C₆hydroxyalkyl, or C(O)OC₁-C₄alkyl.

In further embodiments, R¹ in the compounds of Formula IA or compounds of Formula IB is triazolyl substituted with one or more C₁-C₄alkyl group, C₁-C₄haloalkyl group, C₁-C₆hydroxyalkyl, CO₂(C₁-C₄alkyl), or oxo.

In some embodiments, R¹ in the compounds of Formula IA or compounds of Formula IB is triazolyl substituted with a C₁-C₄alkyl group. In other embodiments, R¹ in the compounds of Formula IA or compounds of Formula IB is triazolyl substituted with a C₁-C₃alkyl group. In further embodiments, R¹ in the compounds of Formula IA or compounds of Formula IB is triazolyl substituted with a methyl group.

In some embodiments, R¹ in the compounds of Formula IA or compounds of Formula IB is 4-methyl-1,2,3-triazol-2-yl. In other embodiments, R¹ in the compounds of Formula IA or compounds of Formula IB is 4-methyl-1,2,3-triazol-1-yl. In further embodiments, R¹ in the compounds of Formula IA or compounds of Formula IB is 1-methyl-1,2,4-triazol-5-yl. In other embodiments, R¹ in the compounds of Formula IA or compounds of Formula IB is 3-methyl-1,2,4-triazol-5-yl.

In some embodiments, R¹ in the compounds of Formula IA or compounds of Formula IB is triazolyl substituted with a C₁-C₄haloalkyl group. In other embodiments, R¹ in the compounds of Formula IA or compounds of Formula IB is triazolyl substituted with a C₁-C₃haloalkyl group. In further embodiments, R¹ in the compounds of Formula IA or compounds of Formula IB is triazolyl substituted with a CF₃, CH₂F, or CHF₂ group.

In some embodiments, R¹ in the compounds of Formula IA or compounds of Formula IB is 4-difluoromethyl-1,2,3-triazol-2-yl. In other embodiments, R¹ in the compounds of Formula IA or compounds of Formula IB is 4-difluoromethyl-1,2,4-triazol-3-yl. In further embodiments, R¹ in the compounds of Formula IA or compounds of Formula IB is 1-difluoromethyl-1,2,4-triazol-3-yl. In other embodiments, R¹ in the compounds of Formula IA or compounds of Formula IB is 1-difluoromethyl-1,2,4-triazol-5-yl.

In some embodiments, R¹ in the compounds of Formula IA or compounds of Formula IB is triazolyl substituted with a C₁-C₆hydroxyalkyl group. In other embodiments, R¹ in the compounds of Formula IA or compounds of Formula IB is triazolyl substituted with a CH₂OH, CH(CH₃)OH, or C(CH₃)₂OH group.

In some embodiments, R¹ in the compounds of Formula IA or compounds of Formula IB is 4-(2-hydroxypropan-2-yl)-1,2,3-triazol-2-yl. In other embodiments, R¹ in the compounds of Formula IA or compounds of Formula IB is 4-hydroxymethyl-1,2,3-triazol-2-yl. In further embodiments, R¹ in the compounds of Formula IA or compounds of Formula IB is 1-hydroxyethyl-1,2,3-triazol-2-yl. In other embodiments, R¹ in the compounds of Formula IA or compounds of Formula IB is 4-hydroxymethyl-1,2,3-triazol-1-yl. In other embodiments, R¹ in the compounds of Formula IA or compounds of Formula IB is 1-hydroxyethyl-1,2,3-triazol-1-yl.

In some embodiments, R¹ in the compounds of Formula IA or compounds of Formula IB is triazolyl substituted with a —C(O)OC₁-C₄alkyl group. In other embodiments, R¹ in the compounds of Formula IA or compounds of Formula IB is triazolyl substituted with a —C(O)OC₁-C₃alkyl group. In further embodiments, R¹ in the compounds of Formula IA or compounds of Formula IB is triazolyl substituted with a methyl carboxylate group, i.e., —C(O)OCH₃.

In some embodiments, R¹ in the compounds of Formula IA or compounds of Formula IB is 4-methyl carboxylate-1,2,3-triazol-2-yl.

In other embodiments, R¹ in the compounds of Formula IA or compounds of Formula IB is 4-ethyl-3-(hydroxymethyl)-5-oxo-4,5-dihydro-1,2,4-triazol-1-yl.

In some embodiments, R¹ in the compounds of Formula IA or compounds of Formula IB is substituted pyrazolyl.

In some embodiments, R¹ in the compounds of Formula IA or compounds of Formula IB is pyrazolyl substituted with one or more C₁-C₄alkyl, C₁-C₄haloalkyl, halo, or NR^{b}R^{c} wherein each R^{b} and R^{c} is independently hydrogen or C₁-C₄alkyl.

In some embodiments, R¹ in the compounds of Formula IA or compounds of Formula IB is pyrazolyl substituted with a C₁-C₄alkyl group. In other embodiments, R¹ in the compounds of Formula IA or compounds of Formula IB is pyrazolyl substituted with a C₁-C₃alkyl group. In further embodiments, R¹ in the compounds of Formula IA or compounds of Formula IB is pyrazolyl substituted with a methyl group.

In some embodiments, R¹ in the compounds of Formula IA or compounds of Formula IB is 1-methylpyrazol-4-yl. In other embodiments, R¹ in the compounds of Formula IA or compounds of Formula IB is 1-methylpyrazol-3-yl. In further embodiments, R¹ in the compounds of Formula IA or compounds of Formula IB is 4-methylpyrazol-3-yl.

In some embodiments, R¹ in the compounds of Formula IA or compounds of Formula IB is pyrazolyl substituted with a C₁-C₄haloalkyl group. In other embodiments, R¹ in the compounds of Formula IA or compounds of Formula IB is pyrazolyl substituted with a C₁-C₃haloalkyl group. In further embodiments, R¹ in the compounds of Formula IA or compounds of Formula IB is pyrazolyl substituted with a CF₃ or CHF₂ group.

In some embodiments, R¹ in the compounds of Formula IA or compounds of Formula IB is 1-CHF₂-pyrazol-3-yl. In other embodiments, R¹ in the compounds of Formula IA or compounds of Formula IB is 5-CF₃-pyrazol-3-yl.

In some embodiments, R¹ in the compounds of Formula IA or compounds of Formula IB is pyrazolyl substituted with a halo group. In other embodiments, R¹ in the compounds of Formula IA or compounds of Formula IB is pyrazolyl substituted with a fluoro group.

In some embodiments, R¹ in the compounds of Formula IA or compounds of Formula IB is 4-fluoropyrazol-1-yl.

In some embodiments, R¹ in the compounds of Formula IA or compounds of Formula IB is pyrazolyl substituted with NR^{b}R^{c}, wherein each R^{b} and R^{c} is independently hydrogen or C₁-C₄alkyl. In other embodiments, R^{b} and R^{c} are hydrogen.

In some embodiments, R¹ in the compounds of Formula IA or compounds of Formula IB is 3-amino-5-fluoropyrazol-1-yl. In other embodiments, R¹ in the compounds of Formula IA or compounds of Formula IB is 3-amino-pyrazol-1-yl.

In some embodiments, R¹ in the compounds of Formula IA or compounds of Formula IB is substituted oxadiazolyl.

In some embodiments, R¹ in the compounds of Formula IA or compounds of Formula IB is oxadiazolyl substituted with an oxo (C═O) group.

In some embodiments, R¹ in the compounds of Formula IA or compounds of Formula IB is 5-oxo-4,5-dihydro-1,2,4-oxadiazol-3-yl. In other embodiments, R¹ in the compounds of Formula IA or compounds of Formula IB is 5-oxo-4,5-dihydro-1,3,4-oxadiazol-2-yl.

In some embodiments, R¹ in the compounds of Formula IA or compounds of Formula IB is thiadiazolyl substituted with a C₁-C₄ group. In other embodiments, R¹ in the compounds of Formula IA or compounds of Formula IB is thiadiazolyl substituted with a C₁-C₃ group. In further embodiments, R¹ in the compounds of Formula IA or compounds of Formula IB is thiadiazolyl substituted with a methyl group.

In some embodiments, R¹ in the compounds of Formula IA or compounds of Formula IB is 3-methyl-1,2,4-thiadiazol-5-yl. In other embodiments, R¹ in the compounds of Formula IA or compounds of Formula IB is 3-methyl-1,2,4-thiadiazol-5-yl.

In some embodiments, R¹ in the compounds of Formula IA or compounds of Formula IB is

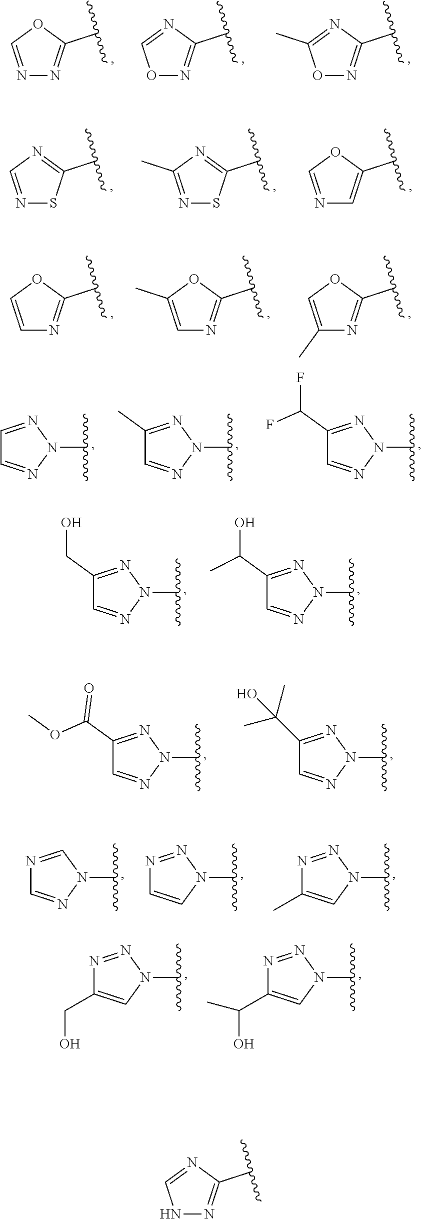

or its tautomer

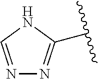 [SMILES: *c1nnc[nH]1]

or its tautomer

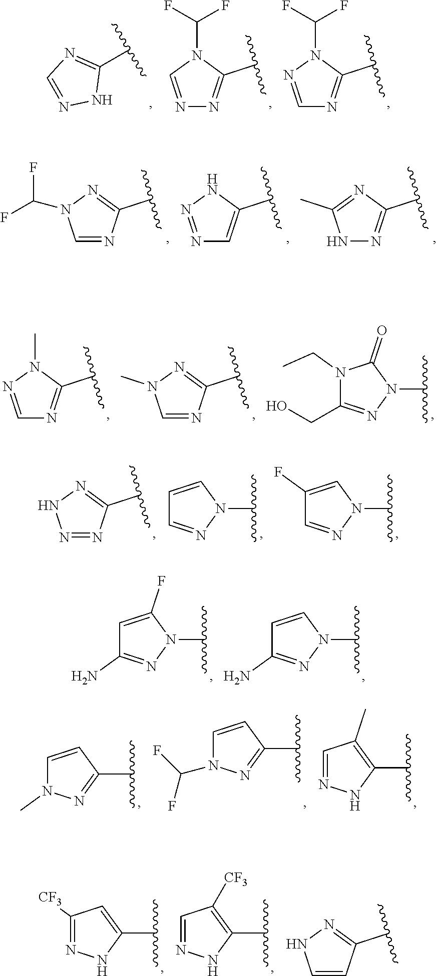

or its tautomer

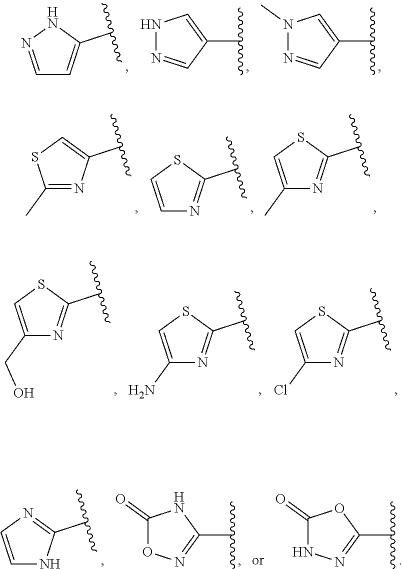

In some embodiments, R¹ in the compounds of Formula IA or compounds of Formula IB is an optionally substituted 6-membered heteroaryl, such as, for example, optionally substituted: pyridinyl, pyrimidinyl, pyrazinyl, or pyridazinyl.

In other embodiments, R¹ in the compounds of Formula IA or compounds of Formula IB is an optionally substituted: pyridinyl, pyrimidinyl, or pyridazinyl.

In some embodiments, R¹ in the compounds of Formula IA or compounds of Formula IB is an unsubstituted 6-membered heteroaryl, such as, for example, an unsubstituted: pyridinyl, pyrimidinyl, pyrazinyl, or pyridazinyl.

In other embodiments, R¹ in the compounds of Formula IA or compounds of Formula IB is a substituted 6-membered heteroaryl, such as, for example, a substituted: pyridinyl, pyrimidinyl, pyrazinyl, or pyridazinyl.

In further embodiments, R¹ in the compounds of Formula IA or compounds of Formula IB is a 6-membered heteroaryl substituted with one or more of NR^{b}R^{c}, C₁-C₄alkyl, C₁-C₄alkoxy, C₁-C₆hydroxyalkyl, cyano, or halo; wherein each R^{b} and R^{c} is independently hydrogen or C₁-C₄alkyl.

In some embodiments, R¹ in the compounds of Formula IA or compounds of Formula IB is

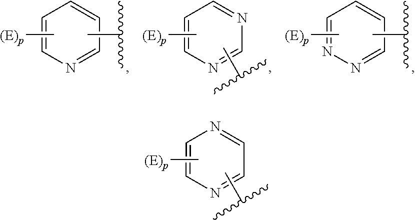

wherein:
each instance of E is independently halo, CN, NO₂, N₃, an optionally substituted alkyl, an optionally substituted alkenyl, an optionally substituted alkynyl, an optionally substituted cycloalkyl, an optionally substituted aryl, an optionally substituted heterocycloalkyl, an optionally substituted heteroaryl, OR^{o1}, SR^{s1}, N(Rⁿ¹)₂, C(═O)N(Rⁿ¹)₂, N(Rⁿ¹)C(═O)R^{c1}, C(═O)R^{c1}, C(═O)OR^{o1}, OC(═O)R^{c1}, S(═O)R^{s1}, S(═O)₂R^{s1}, S(═O)(C₃-C₆cycloalkyl), S(═O)₂(C₃-C₆cycloalkyl), S(═O)OR^{o1}, OS(═O)R^{c1}, S(═O)₂OR^{o1}, OS(═O)₂R^{o1}, S(═O)N(Rⁿ¹)₂, S(═O)₂N(Rⁿ¹)₂, N(Rⁿ¹)S(═O)R^{s1}, N(Rⁿ¹)S(═O)₂R^{s1}, N(Rⁿ¹)C(═O)OR^{o1}, OC(═O)N(Rⁿ¹)₂, N(Rⁿ¹)C(═O)N(Rⁿ¹)₂, N(Rⁿ¹)S(═O)N(Rⁿ¹)₂, N(Rⁿ¹)S(═O)₂N(Rⁿ¹)₂, N(Rⁿ¹)S(═O)OR^{o1}, N(Rⁿ¹)S(═O)₂OR^{o1}, OS(═O)N(Rⁿ¹)₂, or OS(═O)₂N(Rⁿ¹)₂; or alternatively two instances of (E)ₚ attached to adjacent carbon atoms, can be taken together with the carbon atom(s) to which they are attached to form an optionally substituted cycloalkyl or a heterocycloalkyl; wherein:
   each instance of Rⁿ¹ is independently hydrogen, an optionally substituted C₁-C₆alkyl, or a nitrogen protecting group;
   each instance of R^{o1} is independently hydrogen, an optionally substituted C₁-C₆alkyl, or an oxygen protecting group; and
   each instance of R^{o1} is independently an optionally substituted C₁-C₆alkyl; and
   each instance of R^{s1} is independently an optionally substituted C₁-C₆alkyl or a sulfur protecting group; and
p is 0, 1, 2, 3, or 4, as valency permits. Where a particular atom is not substituted with E, it should be understood as having the required number of hydrogens as valency permits so as to form a stable ring.

In some embodiments, R¹ in the compounds of Formula IA or compounds of Formula IB is an unsubstituted pyridinyl.

In some embodiments, R¹ in the compounds of Formula IA or compounds of Formula IB is unsubstituted pyridin-2-yl. In other embodiments, R¹ in the compounds of Formula IA or compounds of Formula IB is unsubstituted pyridin-3-yl. In further embodiments, R¹ in the compounds of Formula IA or compounds of Formula IB is unsubstituted pyridin-4-yl.

In some embodiments, R¹ in the compounds of Formula IA or compounds of Formula IB is an unsubstituted pyrimidinyl.

In some embodiments, R¹ in the compounds of Formula IA or compounds of Formula IB is unsubstituted pyrimidin-2-yl. In other embodiments, R¹ in the compounds of Formula IA or compounds of Formula IB is unsubstituted pyrimidin-4-yl.

In some embodiments, R¹ in the compounds of Formula IA or compounds of Formula IB is an unsubstituted pyridazinyl.

In some embodiments, R¹ in the compounds of Formula IA or compounds of Formula IB is unsubstituted pyridazin-3-yl.

In other embodiments, R¹ in the compounds of Formula IA or compounds of Formula IB is a substituted pyridinyl.

In some embodiments, R¹ in the compounds of Formula IA or compounds of Formula IB is a pyridinyl substituted with one or more of NR^{b}R^{c} (wherein each R^{b} and R^{c} is independently hydrogen or C₁-C₄alkyl), C₁-C₄alkyl, C₁-C₄alkoxy, C₁-C₆hydroxyalkyl, cyano, halo, or oxo.

In some embodiments, R¹ in the compounds of Formula IA or compounds of Formula IB is a pyridinyl substituted with one or more of NR^{b}R^{c}(wherein each R^{b} and R^{c} is independently hydrogen or C₁-C₄alkyl), C₁-C₄alkyl, C₁-C₄alkoxy, C₁-C₆hydroxyalkyl, cyano, or halo.

In some embodiments, R¹ in the compounds of Formula IA or compounds of Formula IB is a pyridinyl substituted with one or more of NH₂, CH₃, OCH₃, CH₂OH, CN, F, or oxo.

In some embodiments, R¹ in the compounds of Formula IA or compounds of Formula IB is a pyridinyl substituted with one or more of NH₂, CH₃, OCH₃, CH₂OH, CN, or F.

In some embodiments, R¹ in the compounds of Formula IA or compounds of Formula IB is a pyridinyl substituted with NH₂.

In some embodiments, R¹ in the compounds of Formula IA or compounds of Formula IB is

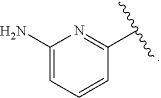 [SMILES: *c1cccc(N)n1]

In some embodiments, R¹ in the compounds of Formula IA or compounds of Formula IB is a pyrimidinyl substituted with NR^{b}R^{c} (wherein each R^{b} and R^{c} is independently hydrogen or C₁-C₄alkyl).

In some embodiments, R¹ in the compounds of Formula IA or compounds of Formula IB is a pyrimidinyl substituted with NH₂ or NH(CH₃).

In some embodiments, R¹ in the compounds of Formula IA or compounds of Formula IB is

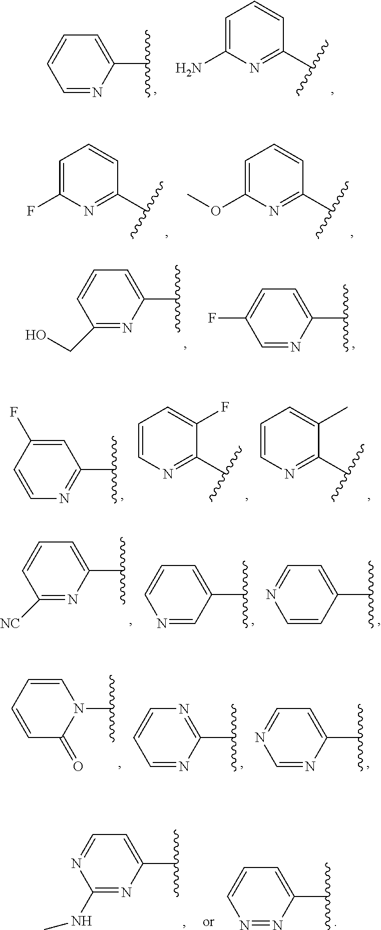

In some embodiments, R¹ in the compounds of Formula IA or compounds of Formula IB is an optionally substituted 9-membered heteroaryl, such as, for example, optionally substituted: benzofuranyl, isobenzofuranyl, benzoxazolyl, benzoisoxazolyl, benzothiazolyl, benzoisothiazolyl, indolyl, isoindolyl, indazolyl, benzimidazolyl, or benzothiophenyl.

In other embodiments, R¹ in the compounds of Formula IA or compounds of Formula IB is an unsubstituted 9-membered heteroaryl, such as, for example, an unsubstituted: benzofuranyl, isobenzofuranyl, benzoxazolyl, benzoisoxazolyl, benzothiazolyl, benzoisothiazolyl, indolyl, isoindolyl, indazolyl, benzimidazolyl, or benzothiophenyl.

In further embodiments, R¹ in the compounds of Formula IA or compounds of Formula IB is an optionally substituted benzofuranyl or optionally substituted benzoxazolyl.

In other embodiments, R¹ in the compounds of Formula IA or compounds of Formula IB is

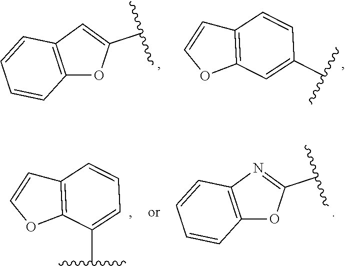

In some embodiments, R¹ in the compounds of Formula IA or compounds of Formula IB is an optionally substituted aryl, such as, for example, optionally substituted phenyl.

In other embodiments, R¹ in the compounds of Formula IA or compounds of Formula IB is an unsubstituted aryl, such as, for example, an unsubstituted phenyl.

In other embodiments, R¹ in the compounds of Formula IA or compounds of Formula IB is a substituted aryl, such as, for example, a substituted phenyl.

In some embodiments, R¹ in the compounds of Formula IA or compounds of Formula IB is

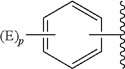

wherein:
each instance of E is independently halo, CN, NO₂, N₃, an optionally substituted alkyl, an optionally substituted alkenyl, an optionally substituted alkynyl, an optionally substituted cycloalkyl, an optionally substituted aryl, an optionally substituted heterocycloalkyl, an optionally substituted heteroaryl, OR^{o1}, SR^{s1}, N(Rⁿ¹)₂, C(═O)N(Rⁿ¹)₂, N(Rⁿ¹)C(═O)R^{c1}, C(═O)R^{o1}, C(═O)OR^{o1}, OC(═O)R^{o1}, S(═O)R^{s1}, S(═O)₂R^{s1}, S(═O)(C₃-C₆cycloalkyl), S(═O)₂(C₃-C₆cycloalkyl), S(═O)OR^{o1}, OS(═O)R^{o1}, S(═O)₂OR^{o1}, OS(═O)₂R^{o1}, S(═O)N(Rⁿ¹)₂, S(═O)₂N(Rⁿ¹)₂, N(Rⁿ¹)S(═O)R^{s1}, N(Rⁿ¹)S(═O)₂R^{s1}, N(Rⁿ¹)C(═O)OR^{o1}, OC(═O)N(Rⁿ¹)₂, N(Rⁿ¹)C(═O)N(Rⁿ¹)₂, N(Rⁿ¹)S(═O)N(Rⁿ¹)₂, N(Rⁿ¹)S(═O)₂N(Rⁿ¹)₂, N(Rⁿ¹)S(═O)OR^{o1}, N(Rⁿ¹)S(═O)₂OR^{o1}, OS(═O)N(Rⁿ¹)₂, or OS(═O)₂N(Rⁿ¹)₂; or alternatively two instances of (E)ₚ attached to adjacent carbon atoms, can be taken together with the carbon atom(s) to which they are attached to form an optionally substituted cycloalkyl or a heterocycloalkyl; wherein:
   each instance of Rⁿ¹ is independently hydrogen, an optionally substituted C₁-C₆alkyl, or a nitrogen protecting group;
   each instance of R^{o1} is independently hydrogen, an optionally substituted C₁-C₆alkyl, or an oxygen protecting group; and
   each instance of R^{c1} is independently an optionally substituted C₁-C₆alkyl; and
   each instance of R^{s1} is independently an optionally substituted C₁-C₆alkyl or a sulfur protecting group; and
p is 0, 1, 2, 3, 4, or 5, as valency permits. Where a particular atom is not substituted with E, it should be understood as having the required number of hydrogens as valency permits so as to form a stable ring.

In some embodiments, R¹ in the compounds of Formula IA or compounds of Formula IB is a substituted aryl, such as, for example, a substituted phenyl, substituted with one or more NR^{b}R^{c} (wherein each R^{b} and R^{c} is independently hydrogen or C₁-C₄alkyl), halo, oxo, cyano, C₁-C₆alkyl, C₁-C₆haloalkyl, C₁-C₆alkoxy, C₁-C₆hydroxyalkyl, or —C(O)OC₁-C₄alkyl.

In other embodiments, R¹ in the compounds of Formula IA or compounds of Formula IB is a phenyl substituted with one or more of C₁-C₆alkyl, C₁-C₆haloalkyl, C₁-C₆hydroxyalkyl, halo, or cyano.

In further embodiments, R¹ in the compounds of Formula IA or compounds of Formula IB is a phenyl substituted with one or more of C₁-C₆hydroxyalkyl, halo, or cyano.

In some embodiments, R¹ in the compounds of Formula IA or compounds of Formula IB is a phenyl substituted with one or more of CH₂OH, F, or cyano.

In further embodiments, R¹ in the compounds of Formula IA or compounds of Formula IB is a phenyl substituted with one or more of C₁-C₆alkyl, C₁-C₆haloalkyl, C₁-C₆hydroxyalkyl, halo, cyano, or C₁-C₆cyanoalkyl.

In yet further embodiments, R¹ in the compounds of Formula IA or compounds of Formula IB is a phenyl substituted with one or more of CH₂OH, F, cyano, or CH₂CN.

In some embodiments, R¹ in the compounds of Formula IA or compounds of Formula IB is

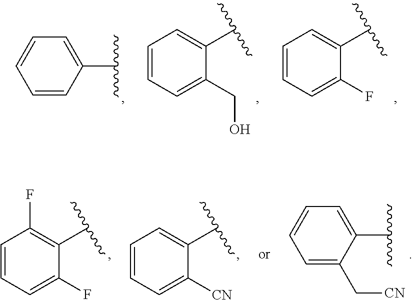

In some aspects, R¹ in the compounds of Formula IA or compounds of Formula IB is optionally substituted imidazolidinonyl, optionally substituted pyrrolidinonyl, optionally substituted dihydropyrazolonyl, optionally substituted dihydropyrrolonyl, optionally substituted dihydroimidazolonyl, or optionally substituted dihydrotriazolonyl.

In some embodiments, R¹ in the compounds of Formula IA or compounds of Formula IB is an unsubstituted imidazolidinonyl, an unsubstituted pyrrolidinonyl, an unsubstituted dihydropyrazolonyl, an unsubstituted dihydropyrrolonyl, an unsubstituted dihydroimidazolonyl, or an unsubstituted dihydrotriazolonyl.

In some embodiments, R¹ in the compounds of Formula IA or compounds of Formula IB is a substituted imidazolidinonyl, a substituted pyrrolidinonyl, a substituted dihydropyrazolonyl, a substituted dihydropyrrolonyl, a substituted dihydroimidazolonyl, or a substituted dihydrotriazolonyl.

In some embodiments, R¹ in the compounds of Formula IA or compounds of Formula IB is

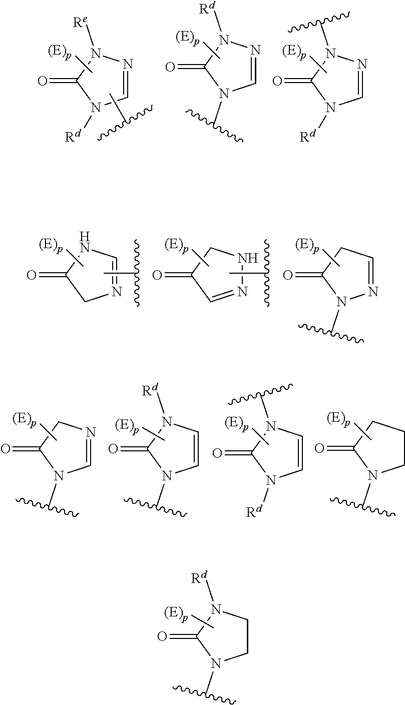

wherein:
each instance of R^{d} and Re is independently hydrogen, optionally substituted C₁-C₆alkyl, or a nitrogen protecting group;
each instance of E is independently halo, CN, NO₂, N₃, an optionally substituted alkyl, an optionally substituted alkenyl, an optionally substituted alkynyl, an optionally substituted cycloalkyl, an optionally substituted aryl, an optionally substituted heterocycloalkyl, an optionally substituted heteroaryl, OR^{o1}, SR^{s1}, N(Rⁿ¹)₂, C(═O)N(Rⁿ¹)₂, N(Rⁿ¹)C(═O)R^{c1}, C(═O)R^{o1}, C(═O)OR^{o1}, OC(═O)R^{o1}, S(═O)R^{s1}, S(═O)₂R^{s1}, S(═O)(C₃-C₆cycloalkyl), S(═O)₂(C₃-C₆cycloalkyl), S(═O)OR^{o1}, OS(═O)R^{o1}, S(═O)₂OR^{o1}, OS(═O)₂R^{c1}, S(═O)N(Rⁿ¹)₂, S(═O)₂N(Rⁿ¹)₂, N(Rⁿ¹)S(═O)R^{s1}, N(Rⁿ¹)S(═O)₂R^{s1}, N(Rⁿ¹)C(═O)OR^{o1}, OC(═O)N(Rⁿ¹)₂, N(Rⁿ¹)C(═O)N(Rⁿ¹)₂, N(Rⁿ¹)S(═O)N(Rⁿ¹)₂, N(Rⁿ¹)S(═O)₂N(Rⁿ¹)₂, N(Rⁿ¹)S(═O)OR^{o1}, N(Rⁿ¹)S(═O)₂OR^{o1}, OS(═O)N(Rⁿ¹)₂, or OS(═O)₂N(Rⁿ¹)₂; or alternatively two instances of (E)ₚ attached to the same or adjacent carbon atoms, can be taken together with the carbon atom(s) to which they are attached to form an optionally substituted cycloalkyl or a heterocycloalkyl; wherein:
   each instance of Rⁿ¹ is independently hydrogen, an optionally substituted C₁-C₆alkyl, or a nitrogen protecting group;
   each instance of R^{o1} is independently hydrogen, an optionally substituted C₁-C₆alkyl, or an oxygen protecting group; and
   each instance of R^{c1} is independently an optionally substituted C₁-C₆alkyl; and
   each instance of R^{s1} is independently an optionally substituted C₁-C₆alkyl or a sulfur protecting group; and
p is 0, 1, 2, 3, or 4, as valency permits. Where a particular atom is not substituted with E, it should be understood as having the required number of hydrogens as valency permits so as to form a stable ring.

In some aspects, R¹ in the compounds of Formula IA or compounds of Formula IB is optionally substituted 4-, 5-, or 6-membered heterocycloalkyl, such as, for example, optionally substituted: azetidinyl, pyrrolidinyl, dioxolanyl, imidazolidinyl, pyrazolidinyl, piperazinyl, piperidinyl, dioxanyl, morpholinyl, thianyl, thianyl sulfoxide, 1-oxo-1-imino-1thiacyclohexyl, dithianyl, thiomorpholinyl, oxetanyl, tetrahydrofuranyl, tetrahydropyranyl, or tetrahydrothiophenyl.

In some embodiments, R¹ in the compounds of Formula IA or compounds of Formula IB is an unsubstituted 4-, 5-, or 6-membered heterocycloalkyl, such as, for example, unsubstituted: azetidinyl, oxetanyl, pyrrolidinyl, dioxolanyl, imidazolidinyl, pyrazolidinyl, piperazinyl, piperidinyl, dioxanyl, morpholinyl, thianyl, dithianyl, thiomorpholinyl, oxetanyl, tetrahydrofuranyl, tetrahydropyranyl, or tetrahydrothiophenyl.

In further embodiments, R¹ in the compounds of Formula IA or compounds of Formula IB is

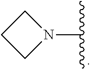 [SMILES: *N1CCC1]

In some embodiments, R¹ in the compounds of Formula IA or compounds of Formula IB is a 4-, 5-, or 6-membered heterocycloalkyl substituted with one or more of oxo, C₁-C₆hydroxyalkyl, C₁-C₆alkyl, C₁-C₆alkoxy(alkylene), C₁-C₆cyanoalkyl, OH, cyano, CONR^{b}R^{c}, wherein each R^{b} and R^{c} is independently hydrogen or C₁-C₄alkyl.

In some embodiments, R¹ in the compounds of Formula IA or compounds of Formula IB is an optionally substituted 4-membered heterocycloalkyl, such as, for example, optionally substituted: azetidinyl or oxetanyl.

In other embodiments, R¹ in the compounds of Formula IA or compounds of Formula IB is an optionally substituted 5-membered heterocycloalkyl, such as, for example, optionally substituted: pyrrolidinyl, dioxolanyl, imidazolidinyl, pyrazolidinyl, tetrahydrofuranyl, or tetrahydrothiophenyl.

In further embodiments, R¹ in the compounds of Formula IA or compounds of Formula IB is an optionally substituted 6-membered heterocycloalkyl, such as, for example, optionally substituted: piperazinyl, piperidinyl, dioxanyl, morpholinyl, thianyl, dithianyl, thiomorpholinyl, or tetrahydropyranyl.

In some aspects, R¹ in the compounds of Formula IA or compounds of Formula IB is optionally substituted imidazolidinonyl, such as, for example,

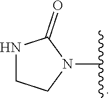 [SMILES: *N1CCNC1=O]

In some embodiments, R¹ in the compounds of Formula IA or compounds of Formula IB is a substituted imidazolidinonyl.

In some aspects, R¹ in the compounds of Formula IA or compounds of Formula IB is an unsubstituted pyrrolidinonyl, such as, for example,

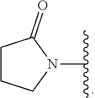 [SMILES: *N1CCCC1=O]

In some embodiments, R¹ in the compounds of Formula IA or compounds of Formula IB is a substituted pyrrolidinonyl.

In some aspects, R¹ in the compounds of Formula IA or compounds of Formula IB is optionally substituted dihydropyrazolonyl.

In some aspects, R¹ in the compounds of Formula IA or compounds of Formula IB is optionally substituted dihydroimidazolonyl.

In some aspects, R¹ in the compounds of Formula IA or compounds of Formula IB is optionally substituted dihydrotriazolonyl.

In some embodiments, R¹ in the compounds of Formula IA or compounds of Formula IB is optionally substituted imidazolidinonyl, optionally substituted pyrrolidinonyl, optionally substituted dihydropyrazolonyl, optionally substituted dihydropyrrolonyl, optionally substituted dihydroimidazolonyl, or optionally substituted dihydrotriazolonyl.

In some embodiments, R¹ in the compounds of Formula IA or compounds of Formula IB is optionally substituted imidazolidinonyl, optionally substituted pyrrolidinyl, or optionally substituted tetrahydrofuranyl.

In other embodiments, R¹ in the compounds of Formula IA or compounds of Formula IB is optionally substituted pyrrolidinyl or optionally substituted tetrahydrofuranyl.

In some embodiments, R¹ in the compounds of Formula IA or compounds of Formula IB is optionally substituted pyrrolidinyl.

In other embodiments, R¹ in the compounds of Formula IA or compounds of Formula IB is unsubstituted pyrrolidinyl, such as, for example,

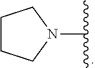 [SMILES: *N1CCCC1]

In further embodiments, R¹ in the compounds of Formula IA or compounds of Formula IB is pyrrolidinyl substituted with one or more of oxo, C₁-C₆hydroxyalkyl, C₁-C₆alkyl, C₁-C₆alkoxy(alkylene), C₁-C₆cyanoalkyl, OH, cyano, CONR^{b}R^{c}, wherein each R^{b} and R^{c} is independently hydrogen or C₁-C₄alkyl.

In other embodiments, R¹ in the compounds of Formula IA or compounds of Formula IB is pyrrolidinyl substituted with one or more of oxo, C(CH₃)₂OH, methyl, CH₂OH, CH₂OCH₃, OH, C(O)NH₂, cyano, or CH₂CN.

In some embodiments, R¹ in the compounds of Formula IA or compounds of Formula IB is

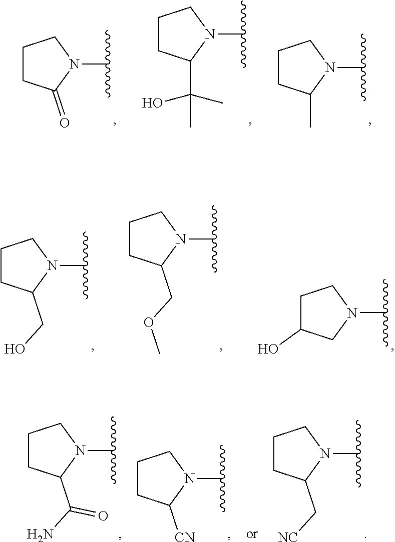

In some embodiments, R¹ in the compounds of Formula IA or compounds of Formula IB is

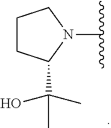 [SMILES: *N1CCC[C@H]1C(C)(C)O]

In some embodiments, R¹ in the compounds of Formula IA or compounds of Formula IB is

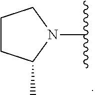 [SMILES: *N1CCC[C@H]1C]

In other embodiments, R¹ in the compounds of Formula IA or compounds of Formula IB is

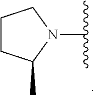 [SMILES: *N1CCC[C@@H]1C]

In some embodiments, R¹ in the compounds of Formula IA or compounds of Formula IB is

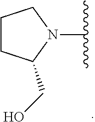 [SMILES: *N1CCC[C@H]1CO]

In further embodiments, R¹ in the compounds of Formula IA or compounds of Formula IB is

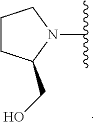 [SMILES: *N1CCC[C@@H]1CO]

In some embodiments, R¹ in the compounds of Formula IA or compounds of Formula IB is

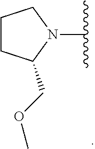 [SMILES: *N1CCC[C@H]1COC]

In other embodiments, R¹ in the compounds of Formula IA or compounds of Formula IB is

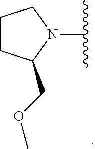 [SMILES: *N1CCC[C@@H]1COC]

In some embodiments, R¹ in the compounds of Formula IA or compounds of Formula IB is optionally substituted tetrahydrofuranyl.

In some embodiments, R¹ in the compounds of Formula IA or compounds of Formula IB is an unsubstituted tetrahydrofuranyl, such as, for example,

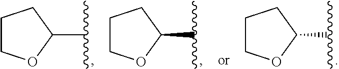

In some embodiments, R¹ in the compounds of Formula IA or compounds of Formula IB is

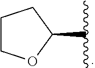 [SMILES: *[C@@H]1CCCO1]

In other embodiments, R¹ in the compounds of Formula IA or compounds of Formula IB is

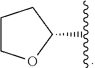 [SMILES: *[C@H]1CCCO1]

In some aspects, Cy in the compounds of Formula IA, Formula IB, Formula IIA, or Formula IIB is optionally substituted C₅-C₇ cycloalkyl or optionally substituted 6- or 7-membered heterocycloalkyl.

It will be understood that the Cy group in the compounds of the disclosure, when unsubstituted, is a diradical with two attachments to other parts of the molecule. As such, the potential exists for Cy to have multiple stereochemical configurations. When no stereochemistry is indicated, such as, for example:

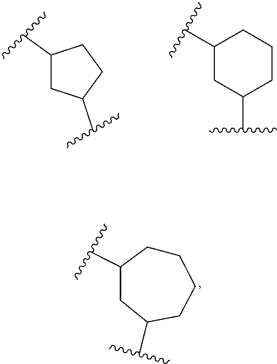 [SMILES: *C1CCC(*)C1 ; *C1CCCC(*)C1 ; *C1CCCCC(*)C1]

the structure is meant to represent all stereoisomeric configurations (e.g., cis and trans) including any mixture of stereoisomers in any proportions. Where a specific stereochemistry is indicated in the structure, then the structure is meant to encompass the stereoisomer with the indicated absolute configuration, but that structure may also contain a mixture of the other enantiomer of the indicated absolute configuration in any ratio. Thus, for example, unless otherwise indicated, the structure

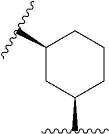 [SMILES: *[C@@H]1CCC[C@H](*)C1]

is meant to represent

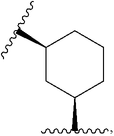 [SMILES: *[C@@H]1CCC[C@H](*)C1]

and may include the enantiomer

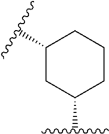 [SMILES: *[C@@H]1CCC[C@H](*)C1]

in any ratio.

In some embodiments, Cy in the compounds of Formula IA, Formula IB, Formula IIA, or Formula IIB is optionally substituted C₅-C₇ cycloalkyl, such as, optionally substituted: C₅cycloalkyl, C₆cycloalkyl, or C₇cycloalkyl.

In some embodiments, Cy in the compounds of Formula IA, Formula IB, Formula IIA, or Formula IIB is optionally substituted cyclopentyl, optionally substituted cyclohexyl, or optionally substituted cycloheptyl.

In some embodiments, Cy in the compounds of Formula IA, Formula IB, Formula IIA, or Formula IIB is optionally substituted C₆ cycloalkyl or optionally substituted C₇ cycloalkyl.

In some embodiments, Cy in the compounds of Formula IA, Formula IB, Formula IIA, or Formula IIB is optionally substituted cyclohexyl or optionally substituted cycloheptyl.

In some embodiments, Cy in the compounds of Formula IA, Formula IB, Formula IIA, or Formula IIB is unsubstituted C₅-C₇ cycloalkyl, such as, unsubstituted: C₅cycloalkyl, C₆cycloalkyl, or C₇cycloalkyl.

In some embodiments, Cy in the compounds of Formula IA, Formula IB, Formula IIA, or Formula IIB is C₅-C₇ cycloalkyl substituted with one or more of OH, deuterium, halo, cyano, C₁-C₃alkyl, C₁-C₃haloalkyl, C₁-C₃alkoxy, deuterated C₁-C₃alkoxy, C₁-C₃haloalkoxy, N₃, NH₂, NH(C₁-C₆alkyl), N(C₁-C₆alkyl)₂, OCO₂(C₁-C₄alkyl), OCO₂(C₃-C₄cycloalkyl), OSO₂(C₁-C₄alkyl), OSO₂(C₃-C₄cycloalkyl), OC(O)(C₁-C₄alkyl), OC(O)(C₃-C₄cycloalkyl), or NR^{m}Rⁿ (wherein R^{m} is hydrogen or C₁-C₄alkyl and Rⁿ is CO₂(C₁-C₄alkyl), C(O)C₁-C₄alkyl, SO₂(C₁-C₄alkyl), or SO₂(C₃-C₄cycloalkyl)).

In some embodiments, Cy in the compounds of Formula IA, Formula IB, Formula IIA, or Formula IIB is C₅-C₇ cycloalkyl substituted with one or more of OH, deuterium, cyano, C₁-C₃alkyl, C₁-C₃haloalkyl, C₁-C₃alkoxy, deuterated C₁-C₃alkoxy, C₁-C₃haloalkoxy, N₃, NH₂, NH(C₁-C₆alkyl), N(C₁-C₆alkyl)₂, OSO₂(C₁-C₄alkyl), OCO₂(C₃-C₄cycloalkyl), OSO₂(C₁-C₄alkyl), OSO₂(C₃-C₄cycloalkyl), OC(O)(C₁-C₄alkyl), OC(O)(C₃-C₄cycloalkyl), or NR^{m}Rⁿ (wherein R^{m} is hydrogen or C₁-C₄alkyl and Rⁿ is CO₂(C₁-C₄alkyl), C(O)C₁-C₄alkyl, SO₂(C₁-C₄alkyl), or SO₂(C₃-C₄cycloalkyl)).

In some embodiments, Cy in the compounds of Formula IA, Formula IB, Formula IIA, or Formula IIB is C₅-C₇ cycloalkyl substituted with one or more OH, deuterium, halo, cyano, C₁-C₃alkyl, C₁-C₃haloalkyl, C₁-C₃alkoxy, deuterated C₁-C₃alkoxy, C₁-C₃haloalkoxy, NH₂, NH(C₁-C₆alkyl), or N(C₁-C₆alkyl)₂.

In some embodiments, Cy in the compounds of Formula IA, Formula IB, Formula IIA, or Formula IIB is C₅-C₇ cycloalkyl substituted with one or more OH, halo, cyano, C₁-C₃alkyl, C₁-C₃haloalkyl, C₁-C₃alkoxy, C₁-C₃haloalkoxy, NH₂, NH(C₁-C₆alkyl), or N(C₁-C₆alkyl)₂.

In some embodiments, Cy in the compounds of Formula IA, Formula IB, Formula IIA, or Formula IIB is C₅-C₇ cycloalkyl substituted with one of OH, C₁-C₃alkoxy, cyano, OSO₂(C₁-C₄alkyl), OSO₂(C₁-C₄alkyl), N₃, or NH₂.

In some embodiments, Cy in the compounds of Formula IA, Formula IB, Formula IIA, or Formula IIB is C₅-C₇ cycloalkyl substituted with one or more OH, C₁-C₃ alkoxy, or cyano.

In some embodiments, Cy in the compounds of Formula IA, Formula IB, Formula IIA, or Formula IIB is C₅-C₇ cycloalkyl substituted with one or more OH or C₁-C₃ alkoxy.

In some embodiments, Cy in the compounds of Formula IA, Formula IB, Formula IIA, or Formula IIB is C₅-C₇ cycloalkyl substituted with one or more OH.

In some embodiments, Cy in the compounds of Formula IA, Formula IB, Formula IIA, or Formula IIB is C₅-C₇ cycloalkyl substituted with one OH.

In some embodiments, Cy in the compounds of Formula IA, Formula IB, Formula IIA, or Formula IIB is C₅-C₇ cycloalkyl substituted with one OH and one C₁-C₃alkyl.

In some embodiments, Cy in the compounds of Formula IA, Formula IB, Formula IIA, or Formula IIB is C₅-C₇ cycloalkyl substituted with one OH and one methyl.

In some embodiments, Cy in the compounds of Formula IA, Formula IB, Formula IIA, or Formula IIB is C₅-C₇ cycloalkyl substituted with one or more C₁-C₃alkoxy.

In some embodiments, Cy in the compounds of Formula IA, Formula IB, Formula IIA, or Formula IIB is C₅-C₇ cycloalkyl substituted with one or more OCH₃.

In some embodiments, Cy in the compounds of Formula IA, Formula IB, Formula IIA, or Formula IIB is C₅-C₇ cycloalkyl substituted with one OCH₃.

In some embodiments, Cy in the compounds of Formula IA, Formula IB, Formula IIA, or Formula IIB is C₅-C₇ cycloalkyl substituted with one or more OCH₂CH₃.

In some embodiments, Cy in the compounds of Formula IA, Formula IB, Formula IIA, or Formula IIB is C₅-C₇ cycloalkyl substituted with one OCH₂CH₃.

In some embodiments, Cy in the compounds of Formula IA, Formula IB, Formula IIA, or Formula IIB is C₅-C₇ cycloalkyl substituted with one or more C₁-C₃haloalkoxy.

In some embodiments, Cy in the compounds of Formula IA, Formula IB, Formula IIA, or Formula IIB is C₅-C₇ cycloalkyl substituted with one or more OCF₃.

In some embodiments, Cy in the compounds of Formula IA, Formula IB, Formula IIA, or Formula IIB is C₅-C₇ cycloalkyl substituted with one OCF₃.

In some embodiments, Cy in the compounds of Formula IA, Formula IB, Formula IIA, or Formula IIB is C₅-C₇ cycloalkyl substituted with one or more OCHF₂.

In some embodiments, Cy in the compounds of Formula IA, Formula IB, Formula IIA, or Formula IIB is C₅-C₇ cycloalkyl substituted with one OCHF₂.

In some embodiments, Cy in the compounds of Formula IA, Formula IB, Formula IIA, or Formula IIB is C₅-C₇ cycloalkyl substituted with one or more deuterated C₁-C₃alkoxy.

In some embodiments, Cy in the compounds of Formula IA, Formula IB, Formula IIA, or Formula IIB is C₅-C₇ cycloalkyl substituted with one or more OCD₃.

In some embodiments, Cy in the compounds of Formula IA, Formula IB, Formula IIA, or Formula IIB is C₅-C₇ cycloalkyl substituted with one OCD₃.

In other embodiments, Cy in the compounds of Formula IA, Formula IB, Formula IIA, or Formula IIB is C₅-C₇ cycloalkyl substituted with one or more F.

In some embodiments, Cy in the compounds of Formula IA, Formula IB, Formula IIA, or Formula IIB is a C₅-C₇ cycloalkyl in which one carbon atom in the cycloalkyl ring is CF₂.

In other embodiments, Cy in the compounds of Formula IA, Formula IB, Formula IIA, or Formula IIB is C₅-C₇ cycloalkyl substituted with one or more cyano.

In other embodiments, Cy in the compounds of Formula IA, Formula IB, Formula IIA, or Formula IIB is C₅-C₇ cycloalkyl substituted with one cyano.

In some embodiments, Cy in the compounds of Formula IA, Formula IB, Formula IIA, or Formula IIB is C₅-C₇ cycloalkyl substituted with one NH₂, NH(C₁-C₆alkyl), N(C₁-C₆alkyl)₂, or NR^{m}Rⁿ (wherein R^{m} is hydrogen or C₁-C₄alkyl and Rⁿ is CO₂(C₁-C₄alkyl), C(O)C₁-C₄alkyl, SO₂(C₁-C₄alkyl), or SO₂(C₃-C₄cycloalkyl)).

In some embodiments, Cy in the compounds of Formula IA, Formula IB, Formula IIA, or Formula IIB is C₅-C₇ cycloalkyl substituted with one N₃.

In some embodiments, Cy in the compounds of Formula IA, Formula IB, Formula IIA, or Formula IIB is C₅-C₇ cycloalkyl substituted with one OCO₂(C₁-C₄alkyl), OCO₂(C₃-C₄cycloalkyl), OSO₂(C₁-C₄alkyl), OSO₂(C₃-C₄cycloalkyl), OC(O)(C₁-C₄alkyl), or OC(O)(C₃-C₄cycloalkyl).

In some embodiments, Cy in the compounds of Formula IA, Formula IB, Formula IIA, or Formula IIB is optionally substituted C₅cycloalkyl.

In some embodiments, Cy in the compounds of Formula IA, Formula IB, Formula IIA, or Formula IIB is optionally substituted cyclopentyl.

In some embodiments, Cy in the compounds of Formula IA, Formula IB, Formula IIA, or Formula IIB is unsubstituted cyclopentyl, such as, for example,

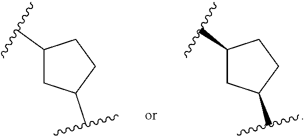 [SMILES: *C1CCC(*)C1 ; *[C@@H]1CC[C@H](*)C1]

In some embodiments, Cy in the compounds of Formula IA, Formula IB, Formula IIA, or Formula IIB is substituted cyclopentyl.

In some embodiments, Cy in the compounds of Formula IA, Formula IB, Formula IIA, or Formula IIB is cyclopentyl substituted with one or more OH, deuterium, halo, cyano, C₁-C₃alkyl, C₁-C₃haloalkyl, C₁-C₃alkoxy, deuterated C₁-C₃alkoxy, C₁-C₃haloalkoxy, NH₂, NH(C₁-C₆alkyl), N(C₁-C₆alkyl)₂.

In some embodiments, Cy in the compounds of Formula IA, Formula IB, Formula IIA, or Formula IIB is cyclopentyl substituted with one or more OH, halo, cyano, —C₁-C₃alkyl, C₁-C₃haloalkyl, C₁-C₃alkoxy, C₁-C₃haloalkoxy, NH₂, NH(C₁-C₆alkyl), or N(C₁-C₆alkyl)₂.

In some embodiments, Cy in the compounds of Formula IA, Formula IB, Formula IIA, or Formula IIB is cyclopentyl substituted with one or more OH.

In some embodiments, Cy in the compounds of Formula IA, Formula IB, Formula IIA, or Formula IIB is cyclopentyl substituted with one OH.

In other embodiments, Cy in the compounds of Formula IA, Formula IB, Formula IIA, or Formula IIB is cyclopentyl substituted with one or more C₁-C₃alkoxy.

In other embodiments, Cy in the compounds of Formula IA, Formula IB, Formula IIA, or Formula IIB is cyclopentyl substituted with one or more C₁-C₃haloalkoxy.

In other embodiments, Cy in the compounds of Formula IA, Formula IB, Formula IIA, or Formula IIB is cyclopentyl substituted with one or more deuterated C₁-C₃alkoxy.

In other embodiments, Cy in the compounds of Formula IA, Formula IB, Formula IIA, or Formula IIB is cyclopentyl substituted with one or more cyano.

In other embodiments, Cy in the compounds of Formula IA, Formula IB, Formula IIA, or Formula IIB is cyclopentyl substituted with one or more F.

In some embodiments, Cy in the compounds of Formula IA, Formula IB, Formula IIA, or Formula IIB is optionally substituted C₆cycloalkyl.

In some embodiments, Cy in the compounds of Formula IA, Formula IB, Formula IIA, or Formula IIB is optionally substituted cyclohexyl.

In some embodiments, Cy in the compounds of Formula IA, Formula IB, Formula IIA, or Formula IIB is unsubstituted cyclohexyl, such as, for example,

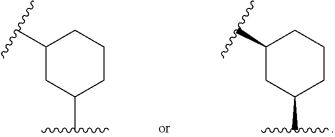 [SMILES: *C1CCCC(*)C1 ; *[C@@H]1CCC[C@H](*)C1]

In some embodiments, Cy in the compounds of Formula IA, Formula IB, Formula IIA, or Formula IIB is substituted cyclohexyl.

In some embodiments, Cy in the compounds of Formula IA, Formula IB, Formula IIA, or Formula IIB is cyclohexyl substituted with one or more of OH, deuterium, halo, cyano, C₁-C₃alkyl, C₁-C₃haloalkyl, C₁-C₃alkoxy, deuterated C₁-C₃alkoxy, C₁-C₃haloalkoxy, N₃, NH₂, NH(C₁-C₆alkyl), N(C₁-C₆alkyl)₂, OSO₂(C₁-C₄alkyl), OCO₂(C₃-C₄cycloalkyl), OSO₂(C₁-C₄alkyl), OSO₂(C₃-C₄cycloalkyl), OC(O)(C₁-C₄alkyl), OC(O)(C₃-C₄cycloalkyl), or NR^{m}Rⁿ (wherein R^{m} is hydrogen or C₁-C₄alkyl and Rⁿ is CO₂(C₁-C₄alkyl), C(O)C₁-C₄alkyl, SO₂(C₁-C₄alkyl), or SO₂(C₃-C₄cycloalkyl)).

In some embodiments, Cy in the compounds of Formula IA, Formula IB, Formula IIA, or Formula IIB is cyclohexyl substituted with one or more OH, deuterium, halo, cyano, C₁-C₃alkyl, C₁-C₃haloalkyl, C₁-C₃alkoxy, deuterated C₁-C₃alkoxy, C₁-C₃haloalkoxy, NH₂, NH(C₁-C₆alkyl), or N(C₁-C₆alkyl)₂.

In some embodiments, Cy in the compounds of Formula IA, Formula IB, Formula IIA, or Formula IIB is cyclohexyl substituted with one or more OH, halo, cyano, C₁-C₃alkyl, C₁-C₃haloalkyl, C₁-C₃alkoxy, C₁-C₃haloalkoxy, NH₂, NH(C₁-C₆alkyl), or N(C₁-C₆alkyl)₂.

In some embodiments, Cy in the compounds of Formula IA, Formula IB, Formula IIA, or Formula IIB is cyclohexyl substituted with one of OH, C₁-C₃alkoxy, cyano, OCO₂(C₁-C₄alkyl), OSO₂(C₁-C₄alkyl), N₃, or N₁₂.

In some embodiments, Cy in the compounds of Formula IA, Formula IB, Formula IIA, or Formula IIB is cyclohexyl substituted with one or more OH, C₁-C₃alkoxy, or cyano.

In some embodiments, Cy in the compounds of Formula IA, Formula IB, Formula IIA, or Formula IIB is cyclohexyl substituted with one or more OH or C₁-C₃alkoxy.

In some embodiments Cy in the compounds of Formula IA, Formula IB, Formula IIA, or Formula IIB is cyclohexyl substituted with one or more OH.

In some embodiments, Cy in the compounds of Formula IA, Formula IB, Formula IIA, or Formula IIB is:

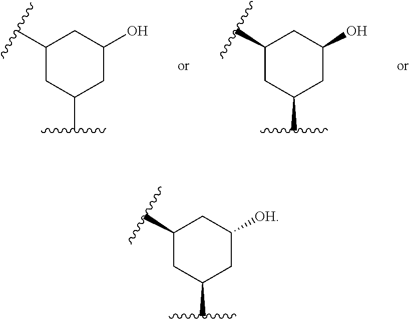 [SMILES: *C1CC(*)CC(O)C1 ; *[C@@H]1C[C@H](*)C[C@H](O)C1 ; *[C@@H]1C[C@H](*)C[C@@H](O)C1]

In some embodiments, Cy in the compounds of Formula IA, Formula IB, Formula IIA, or Formula IIB is:

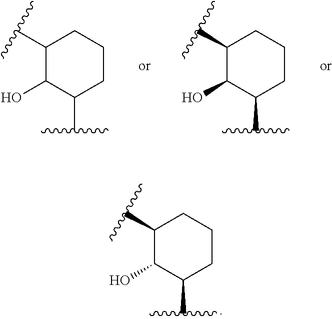 [SMILES: *C1CCCC(*)C1O ; *[C@@H]1CCC[C@H](*)[C@@H]1O ; *[C@@H]1CCC[C@H](*)[C@H]1O]

In some embodiments, Cy in the compounds of Formula IA, Formula IB, Formula IIA, or Formula IIB is cyclohexyl substituted with one OH.

In some embodiments, Cy in the compounds of Formula IA, Formula IB, Formula IIA, or Formula IIB is cyclohexyl substituted with one OH and one C₁-C₃alkyl.

In some embodiments, Cy in the compounds of Formula IA, Formula IB, Formula IIA, or Formula IIB is cyclohexyl substituted with one OH and one methyl.

In some embodiments, Cy in the compounds of Formula IA, Formula IB, Formula IIA, or Formula IIB is:

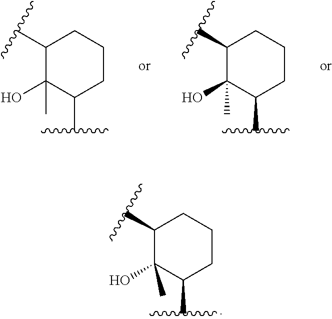

In some embodiments, Cy in the compounds of Formula IA, Formula IB, Formula IIA, or Formula IIB is cyclohexyl substituted with one or more C₁-C₃alkoxy.

In some embodiments, Cy in the compounds of Formula IA, Formula IB, Formula IIA, or Formula IIB is cyclohexyl substituted with one or more OCH₃.

In some embodiments, Cy in the compounds of Formula IA, Formula IB, Formula IIA, or Formula IIB is cyclohexyl substituted with one OCH₃.

In some embodiments, Cy in the compounds of Formula IA, Formula IB, Formula IIA, or Formula IIB is cyclohexyl substituted with one or more OCH₂CH₃.

In some embodiments, Cy in the compounds of Formula IA, Formula IB, Formula IIA, or Formula IIB is:

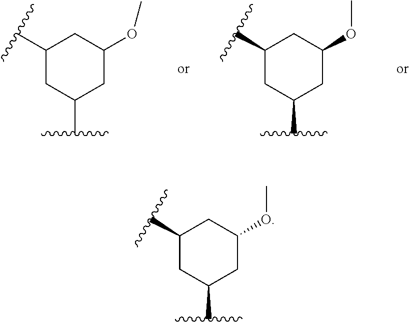

In some embodiments, Cy in the compounds of Formula IA, Formula IB, Formula IIA, or Formula IIB is:

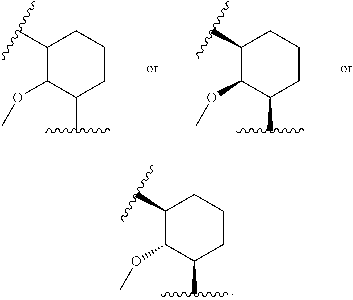

In some embodiments, Cy in the compounds of Formula IA, Formula IB, Formula IIA, or Formula IIB is cyclohexyl substituted with one or more C₁-C₃haloalkoxy.

In some embodiments, Cy in the compounds of Formula IA, Formula IB, Formula IIA, or Formula IIB is cyclohexyl substituted with one or more OCF₃.

In some embodiments, Cy in the compounds of Formula IA, Formula IB, Formula IIA, or Formula IIB is cyclohexyl substituted with one or more OCHF₂.

In some embodiments, Cy in the compounds of Formula IA, Formula IB, Formula IIA, or Formula IIB is cyclohexyl substituted with one or more deuterated C₁-C₃alkoxy.

In some embodiments, Cy in the compounds of Formula IA, Formula IB, Formula IIA, or Formula IIB is cyclohexyl substituted with one or more OCD₃.

In other embodiments, Cy in the compounds of Formula IA, Formula IB, Formula IIA, or Formula IIB is cyclohexyl substituted with one or more F.

In some embodiments, Cy in the compounds of Formula IA, Formula IB, Formula IIA, or Formula IIB is:

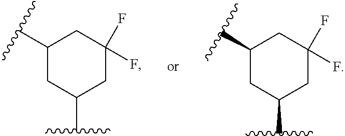 [SMILES: *C1CC(*)CC(F)(F)C1 ; *[C@@H]1C[C@H](*)CC(F)(F)C1]

In other embodiments, Cy in the compounds of Formula IA, Formula IB, Formula IIA, or Formula IIB is cyclohexyl substituted with one or more cyano.

In some embodiments, Cy in the compounds of Formula IA, Formula IB, Formula IIA, or Formula IIB is:

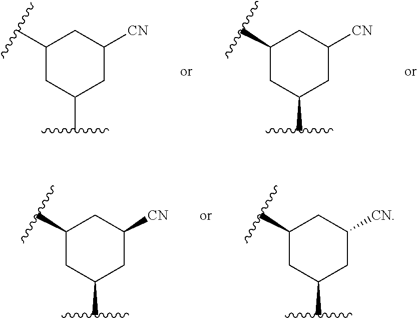 [SMILES: *C1CC(*)CC(C#N)C1 ; *[C@@H]1CC(C#N)C[C@H](*)C1 ; *[C@@H]1C[C@H](*)C[C@H](C#N)C1 ; *[C@@H]1C[C@H](*)C[C@@H](C#N)C1]

In other embodiments, Cy in the compounds of Formula IA, Formula IB, Formula IIA, or Formula IIB is:

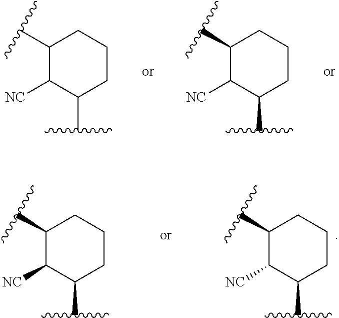 [SMILES: *C1CCCC(*)C1C#N ; *[C@@H]1CCC[C@H](*)C1C#N ; *[C@@H]1CCC[C@H](*)[C@@H]1C#N ; *[C@@H]1CCC[C@H](*)[C@H]1C#N]

In other embodiments, Cy in the compounds of Formula IA, Formula IB, Formula IIA, or Formula IIB is cyclohexyl substituted with one or more NH₂.

In some embodiments, Cy in the compounds of Formula IA, Formula IB, Formula IIA, or Formula IIB is:

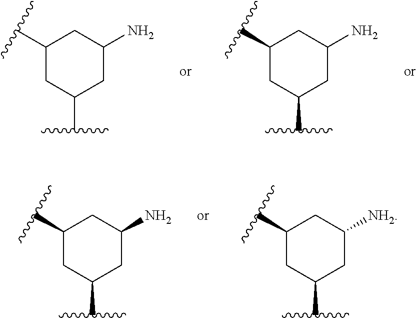 [SMILES: *C1CC(*)CC(N)C1 ; *[C@@H]1CC(N)C[C@H](*)C1 ; *[C@@H]1C[C@H](*)C[C@H](N)C1 ; *[C@@H]1C[C@H](*)C[C@@H](N)C1]

In other embodiments, Cy in the compounds of Formula IA, Formula IB, Formula IIA, or Formula IIB is:

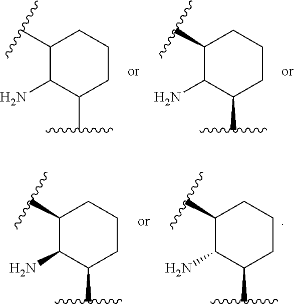 [SMILES: *C1CCCC(*)C1N ; *[C@@H]1CCC[C@H](*)C1N ; *[C@@H]1CCC[C@H](*)[C@@H]1N ; *[C@@H]1CCC[C@H](*)[C@H]1N]

In some embodiments, Cy in the compounds of Formula IA, Formula IB, Formula IIA, or Formula IIB is optionally substituted C₇cycloalkyl.

In some embodiments, Cy in the compounds of Formula IA, Formula IB, Formula IIA, or Formula IIB is optionally substituted cycloheptyl.

In some embodiments, Cy in the compounds of Formula IA, Formula IB, Formula IIA, or Formula IIB is unsubstituted cycloheptyl, such as, for example,

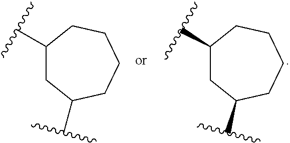 [SMILES: *C1CCCCC(*)C1 ; *[C@@H]1CCCC[C@H](*)C1]

In some embodiments, Cy in the compounds of Formula IA, Formula IB, Formula IIA, or Formula IIB is substituted cycloheptyl.

In some embodiments, Cy in the compounds of Formula IA, Formula IB, Formula IIA, or Formula IIB is cycloheptyl substituted with one or more of OH, deuterium, halo, cyano, C₁-C₃alkyl, C₁-C₃haloalkyl, C₁-C₃alkoxy, deuterated C₁-C₃alkoxy, C₁-C₃haloalkoxy, N₃, NH₂, NH(C₁-C₆alkyl), N(C₁-C₆alkyl)₂, OSO₂(C₁-C₄alkyl), OCO₂(C₃-C₄cycloalkyl), OSO₂(C₁-C₄alkyl), OSO₂(C₃-C₄cycloalkyl), OC(O)(C₁-C₄alkyl), OC(O)(C₃-C₄cycloalkyl), or NR^{m}Rⁿ (wherein R^{m} is hydrogen or C₁-C₄alkyl and Rⁿ is CO₂(C₁-C₄alkyl), C(O)C₁-C₄alkyl, SO₂(C₁-C₄alkyl), or SO₂(C₃-C₄cycloalkyl)).

In some embodiments, Cy in the compounds of Formula IA, Formula IB, Formula IIA, or Formula IIB is cycloheptyl substituted with one or more of OH, deuterium, cyano, C₁-C₃alkyl, C₁-C₃haloalkyl, C₁-C₃alkoxy, deuterated C₁-C₃alkoxy, C₁-C₃haloalkoxy, N₃, NH₂, NH(C₁-C₆alkyl), N(C₁-C₆alkyl)₂, OCO₂(C₁-C₄alkyl), OSO₂(C₃-C₄cycloalkyl), OSO₂(C₁-C₄alkyl), OSO₂(C₃-C₄cycloalkyl), OC(O)(C₁-C₄alkyl), OC(O)(C₃-C₄cycloalkyl), or NR^{m}Rⁿ (wherein R^{m} is hydrogen or C₁-C₄alkyl and Rⁿ is CO₂(C₁-C₄alkyl), C(O)C₁-C₄alkyl, SO₂(C₁-C₄alkyl), or SO₂(C₃-C₄cycloalkyl)).

In some embodiments, Cy in the compounds of Formula IA, Formula IB, Formula IIA, or Formula IIB is cycloheptyl substituted with one or more OH, deuterium, halo, cyano, C₁-C₃alkyl, C₁-C₃haloalkyl, C₁-C₃alkoxy, deuterated C₁-C₃alkoxy, C₁-C₃haloalkoxy, NH₂, NH(C₁-C₆alkyl), N(C₁-C₆alkyl)₂.

In some embodiments, Cy in the compounds of Formula IA, Formula IB, Formula IIA, or Formula IIB is cycloheptyl substituted with one or more OH, halo, cyano, C₁-C₃alkyl, C₁-C₃haloalkyl, C₁-C₃alkoxy, C₁-C₃haloalkoxy, NH₂, NH(C₁-C₆alkyl), or N(C₁-C₆alkyl)₂.

In some embodiments, Cy in the compounds of Formula IA, Formula IB, Formula IIA, or Formula IIB is cycloheptyl substituted with one or more OH.

In some embodiments, Cy in the compounds of Formula IA, Formula IB, Formula IIA, or Formula IIB is cycloheptyl substituted with one OH.

In some embodiments, Cy in the compounds of Formula IA, Formula IB, Formula IIA, or Formula IIB is:

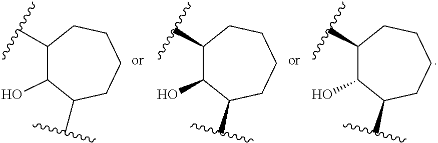 [SMILES: *C1CCCCC(*)C1O ; *[C@@H]1CCCC[C@H](*)[C@@H]1O ; *[C@@H]1CCCC[C@H](*)[C@H]1O]

In some embodiments, Cy in the compounds of Formula IA, Formula IB, Formula IIA, or Formula IIB is cycloheptyl substituted with one or more C₁-C₃alkoxy.

In some embodiments, Cy in the compounds of Formula IA, Formula IB, Formula IIA, or Formula IIB is cycloheptyl substituted with one or more cyano.

In other embodiments, Cy in the compounds of Formula IA, Formula IB, Formula IIA, or Formula IIB is cycloheptyl substituted with one or more F.

In other embodiments, Cy in the compounds of Formula IA, Formula IB, Formula IIA, or Formula IIB is cycloheptyl substituted with one or more NH₂.

In some embodiments, Cy in the compounds of Formula IA, Formula IB, Formula IIA, or Formula IIB is optionally substituted 6- or 7-membered heterocycloalkyl.

In some embodiments, Cy in the compounds of Formula IA, Formula IB, Formula IIA, or Formula IIB is an optionally substituted 6-membered heterocycloalkyl, such as, for example, optionally substituted: piperidinyl, tetrahydropyranyl, thianyl, thianyl sulfone, thianyl sulfoxide, or 1-oxo-1-imino-1-thiacyclohexyl.

In some embodiments, Cy in the compounds of Formula IA, Formula IB, Formula IIA, or Formula IIB is a substituted 6-membered heterocycloalkyl, such as, for example, a substituted: piperidinyl, tetrahydropyranyl, thianyl, thianyl sulfone, thianyl sulfoxide, or 1-oxo-1-imino-1-thiacyclohexyl.

In some embodiments, Cy in the compounds of Formula IA, Formula IB, Formula IIA, or Formula IIB is an unsubstituted 6-membered heterocycloalkyl, such as, for example, an unsubstituted: piperidinyl, tetrahydropyranyl, thianyl, thianyl sulfone, thianyl sulfoxide, or 1-oxo-1-imino-1-thiacyclohexyl.

In some embodiments, Cy in the compounds of Formula IA, Formula IB, Formula IIA, or Formula IIB is unsubstituted piperidinyl, such as, for example,

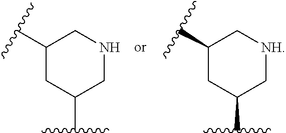 [SMILES: *C1CNCC(*)C1 ; *[C@@H]1CNC[C@H](*)C1]

In some embodiments, Cy in the compounds of Formula IA, Formula IB, Formula IIA, or Formula IIB is optionally substituted piperidinyl.

In some embodiments, Cy in the compounds of Formula IA, Formula IB, Formula IIA, or Formula IIB is piperidinyl, wherein the nitrogen in the piperidinyl is substituted with C₁-C₆alkyl, C(O)C₁-C₆alkyl, C₁-C₆hydroxyalkyl, C(O)NR^{b}R^{c}, CO₂(C₁-C₆alkyl), SO₂(C₁-C₆alkyl), or SO₂(C₃-C₆cycloalkyl); and wherein each R^{b} and R^{c} is independently hydrogen or C₁-C₄alkyl.

In some embodiments, Cy in the compounds of Formula IA, Formula IB, Formula IIA, or Formula IIB is piperidinyl substituted with C₁-C₆alkyl, —C(O)C₁-C₆alkyl, C₁-C₆hydroxyalkyl, C(O)NR^{b}R^{c}, oxo, OH, or C₁-C₆alkoxy, wherein each R^{b} and R^{c} is independently hydrogen or C₁-C₄alkyl.

In some embodiments, Cy in the compounds of Formula IA, Formula IB, Formula IIA, or Formula IIB is piperidinyl substituted with C₁-C₆alkyl, —C(O)C₁-C₆alkyl, or C₁-C₆hydroxyalkyl.

In some embodiments, Cy in the compounds of Formula IA, Formula IB, Formula IIA, or Formula IIB is piperidinyl, wherein the nitrogen in the piperidinyl is substituted with CH₃, C(O)CH₃, C(O)NH₂, CO₂CH₃, or SO₂CH₃.

In some embodiments, Cy in the compounds of Formula IA, Formula IB, Formula IIA, or Formula IIB is piperidinyl substituted with methyl, such as, for example,

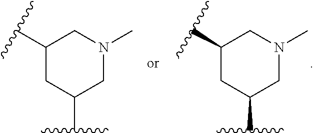

In other embodiments, Cy in the compounds of Formula IA, Formula IB, Formula IIA, or Formula IIB is piperidinyl substituted with acetyl, such as, for example,

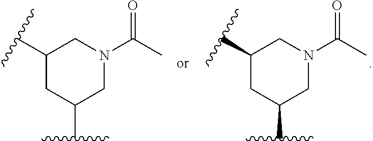 [SMILES: *C1CC(*)CN(C(C)=O)C1 ; *[C@@H]1C[C@H](*)CN(C(C)=O)C1]

In other embodiments, Cy in the compounds of Formula IA, Formula IB, Formula IIA, or Formula IIB is piperidinyl substituted with C₁-C₆hydroxyalkyl, such as, for example,

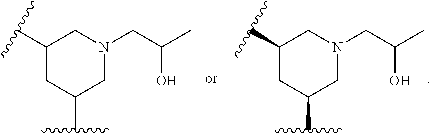 [SMILES: *C1CC(*)CN(CC(C)O)C1 ; *[C@@H]1C[C@H](*)CN(CC(C)O)C1]

In other embodiments, Cy in the compounds of Formula IA, Formula IB, Formula IIA, or Formula IIB is piperidinyl substituted with C(O)NR^{b}R^{c}, wherein each R^{b} and R^{c} is independently hydrogen or C₁-C₄alkyl, such as, for example,

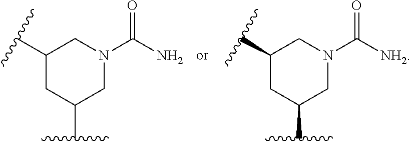 [SMILES: *C1CC(*)CN(C(N)=O)C1 ; *[C@@H]1C[C@H](*)CN(C(N)=O)C1]

In other embodiments, Cy in the compounds of Formula IA, Formula IB, Formula IIA, or Formula IIB is piperidinyl substituted with SO₂C₁-C₆alkyl, such as, for example,

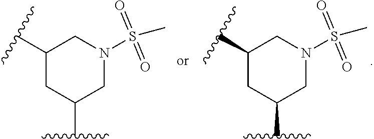 [SMILES: *C1CC(*)CN(S(C)(=O)=O)C1 ; *[C@@H]1C[C@H](*)CN(S(C)(=O)=O)C1]

In further embodiments, Cy in the compounds of Formula IA, Formula IB, Formula IIA, or Formula IIB is piperidinyl substituted with C(O)O C₁-C₆alkyl, such as, for example,

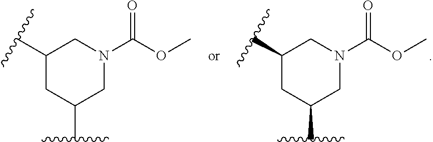 [SMILES: *C1CC(*)CN(C(=O)OC)C1 ; *[C@@H]1C[C@H](*)CN(C(=O)OC)C1]

In some embodiments, Cy in the compounds of Formula IA, Formula IB, Formula IIA, or Formula IIB is unsubstituted tetrahydropyranyl, such as, for example,

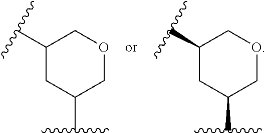 [SMILES: *C1COCC(*)C1 ; *[C@@H]1COC[C@H](*)C1]

In some embodiments, Cy in the compounds of Formula IA, Formula IB, Formula IIA, or Formula IIB is optionally substituted tetrahydropyranyl.

In some embodiments, Cy in the compounds of Formula IA, Formula IB, Formula IIA, or Formula IIB is unsubstituted thianyl, such as, for example,

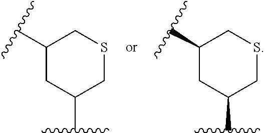 [SMILES: *C1CSCC(*)C1 ; *[C@@H]1CSC[C@H](*)C1]

In some embodiments, Cy in the compounds of Formula IA, Formula IB, Formula IIA, or Formula IIB is optionally substituted thianyl.

In some embodiments, Cy in the compounds of Formula IA, Formula IB, Formula IIA, or Formula IIB is unsubstituted thianyl sulfone, such as, for example,

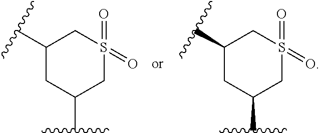 [SMILES: *C1CC(*)CS(=O)(=O)C1 ; *[C@@H]1C[C@H](*)CS(=O)(=O)C1]

In some embodiments, Cy in the compounds of Formula IA, Formula IB, Formula IIA, or Formula IIB is optionally substituted thianyl sulfone.

In some embodiments, Cy in the compounds of Formula IA, compounds of Formula IB, compounds of Formula IIA, or compounds of Formula IIB is unsubstituted thianyl sulfoxide, such as, for example,

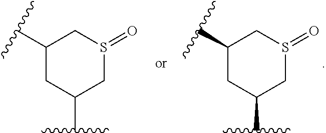 [SMILES: *C1CC(*)CS(=O)C1 ; *[C@@H]1C[C@H](*)CS(=O)C1]

In some embodiments, Cy in the compounds of Formula IA, Formula IB, Formula IIA, or Formula IIB is optionally substituted thianyl sulfoxide.

In some embodiments, Cy in the compounds of Formula IA, Formula IB, Formula IIA, or Formula IIB is unsubstituted 1-oxo-1-imino-1-thiacyclohexyl, such as, for example,

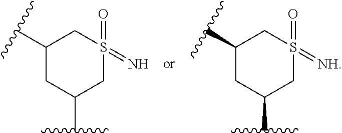 [SMILES: *C1CC(*)CS(=N)(=O)C1 ; *[C@@H]1C[C@H](*)CS(=N)(=O)C1]

In some embodiments, Cy in the compounds of Formula IA, Formula IB, Formula IIA, or Formula IIB is optionally substituted 1-oxo-1-imino-1-thiacyclohexyl.

In some embodiments, Cy in the compounds of Formula IA, Formula IB, Formula IIA, or Formula IIB is an optionally substituted 7-membered heterocycloalkyl, such as, for example, an optionally substituted oxazepanyl, an optionally substituted oxepanyl, an optionally substituted dioxepanyl, an optionally substituted diazepanyl, an optionally substituted thiepanyl, an optionally substituted thiepanyl-1-oxide, an optionally substituted thiepanyl-1,1-dioxide, an optionally substituted azepanyl, or an optionally substituted 1-imino-1-oxide-thiepanyl.

In some embodiments, Cy in the compounds of Formula IA, Formula IB, Formula IIA, or Formula IIB is a substituted 7-membered heterocycloalkyl, such as, for example, a substituted oxazepanyl, a substituted oxepanyl, a substituted dioxepanyl, a substituted azepanyl, a substituted diazepanyl, a substituted thiepanyl, a substituted thiepanyl-1-oxide, a substituted thiepanyl-1,1-dioxide, or a substituted 1-imino-1-oxide-thiepanyl.

In some embodiments, Cy in the compounds of Formula IA, Formula IB, Formula IIA, or Formula IIB is an unsubstituted 7-membered heterocycloalkyl, such as, for example, an unsubstituted oxazepanyl, an unsubstituted oxepanyl, an unsubstituted dioxepanyl, an unsubstituted azepanyl, an unsubstituted diazepanyl, an unsubstituted thiepanyl, an unsubstituted thiepanyl-1-oxide, an unsubstituted thiepanyl-1,1-dioxide, or an unsubstituted 1-imino-1-oxide-thiepanyl.

In some embodiments, Cy in the compounds of Formula IA, Formula IB, Formula IIA, or Formula IIB is an optionally substituted optionally substituted oxazepanyl, an optionally substituted thiepanyl, an optionally substituted thiepanyl-1-oxide, an optionally substituted thiepanyl-1,1-dioxide, an optionally substituted azepanyl, or an optionally substituted 1-imino-1-oxide-thiepanyl.

In some embodiments, Cy in the compounds of Formula IA, Formula IB, Formula IIA, or Formula IIB is an unsubstituted oxazepanyl, such as, for example,

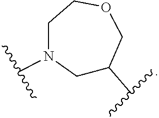 [SMILES: *C1COCCN(*)C1]

In some embodiments, Cy in the compounds of Formula IA, Formula IB, Formula IIA, or Formula IIB is a substituted oxazepanyl.

In some embodiments, Cy in the compounds of Formula IA, Formula IB, Formula IIA, or Formula IIB is an unsubstituted oxepanyl, such as, for example,

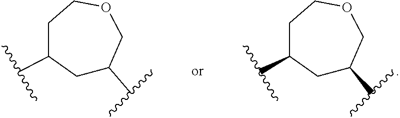 [SMILES: *C1CCOCC(*)C1 ; *[C@@H]1COCC[C@H](*)C1]

In some embodiments, Cy in the compounds of Formula IA, Formula IB, Formula IIA, or Formula IIB is a substituted oxepanyl.

In some embodiments, Cy in the compounds of Formula IA, Formula IB, Formula IIA, or Formula IIB is an unsubstituted dioxepanyl, such as, for example,

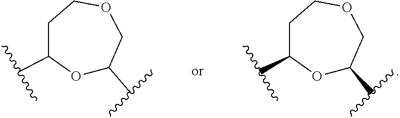 [SMILES: *C1CCOCC(*)O1 ; *[C@@H]1COCC[C@H](*)O1]

In some embodiments, Cy in the compounds of Formula IA, Formula IB, Formula IIA, or Formula IIB is a substituted dioxepanyl.

In some embodiments, Cy in the compounds of Formula IA, Formula IB, Formula IIA, or Formula IIB is an unsubstituted azepanyl, such as for example,

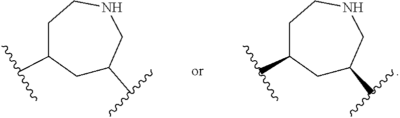 [SMILES: *C1CCNCC(*)C1 ; *[C@@H]1CNCC[C@H](*)C1]

In some embodiments, Cy in the compounds of Formula IA, Formula IB, Formula IIA, or Formula IIB is a substituted azepanyl.

In some embodiments, Cy in the compounds of Formula IA, Formula IB, Formula IIA, or Formula IIB is an unsubstituted diazepanyl, such as, for example,

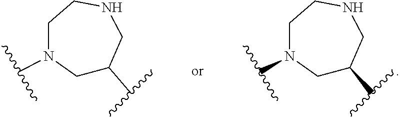

In some embodiments, Cy in the compounds of Formula IA, Formula IB, Formula IIA, or Formula IIB is a substituted diazepanyl.

In some embodiments, Cy in the compounds of Formula IA, Formula IB, Formula IIA, or Formula IIB is an unsubstituted thiepanyl, such as, for example,

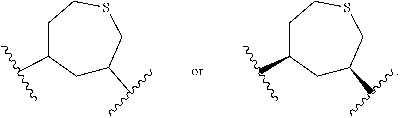 [SMILES: *C1CCSCC(*)C1 ; *[C@@H]1CSCC[C@H](*)C1]

In some embodiments, Cy in the compounds of Formula IA, Formula IB, Formula IIA, or Formula IIB is a substituted thiepanyl.

In some embodiments, Cy in the compounds of Formula IA, Formula IB, Formula IIA, or Formula IIB is an unsubstituted thiepanyl-1-oxide, such as, for example,

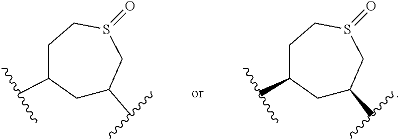 [SMILES: *C1CCS(=O)CC(*)C1 ; *[C@H]1CCS(=O)C[C@@H](*)C1]

In some embodiments, Cy in the compounds of Formula IA, Formula IB, Formula IIA, or Formula IIB is a substituted thiepanyl-1-oxide.

In some embodiments, Cy in the compounds of Formula IA, Formula IB, Formula IIA, or Formula IIB is an unsubstituted thiepanyl-1,1-dioxide, such as, for example,

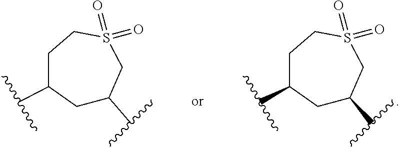 [SMILES: *C1CCS(=O)(=O)CC(*)C1 ; *[C@H]1CCS(=O)(=O)C[C@@H](*)C1]

In some embodiments, Cy in the compounds of Formula IA, Formula IB, Formula IIA, or Formula IIB is a substituted thiepanyl-1,1-dioxide.

In some embodiments, Cy in the compounds of Formula IA, Formula IB, Formula IIA, or Formula IIB is an unsubstituted 1-imino-1-oxide-thiepanyl, such as, for example,

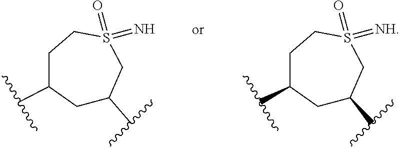 [SMILES: *C1CCS(=N)(=O)CC(*)C1 ; *[C@H]1CCS(=N)(=O)C[C@@H](*)C1]

In some embodiments, Cy in the compounds of Formula IA, Formula IB, Formula IIA, or Formula IIB is a substituted 1-imino-1-oxide-thiepanyl.

In some aspects, L in the compounds of Formula IA, Formula IB, Formula IIA, Formula IIB, Formula IIIA, Formula IIIB, Formula IIIC, Formula IIID, Formula IVA, Formula IVB, Formula VA, Formula VB, Formula VC, or Formula VD is a bond, —O—, —NR^{a}, or optionally substituted C₁-C₄alkylene, wherein R^{a} is hydrogen or C₁-C₄alkyl.

In some embodiments, L is a bond. As used here, “bond” refers to a covalent chemical bond.

In some embodiments, L is —O—.

In some embodiments, L is —NR^{a}—, wherein R^{a} is hydrogen or C₁-C₄alkyl. In embodiments in which L is —NR^{a}— and R^{a} is hydrogen, L is —NH—. In embodiments in which L is —NR^{a}— and R^{a} is C₁-C₆alkyl, L is —N(C₁-C₆alkyl)-, such as, for example, —N(C₆alkyl)-, —N(C₅alkyl)-, —N(C₄alkyl)-, —N(C₃alkyl)-, —N(C₂alkyl)- (e.g., —N(CH₂CH₃)—), —N(C₁alkyl)- (e.g., —N(CH₃)—), and the like.

In some embodiments, L is optionally substituted C₁-C₄alkylene, such as, for example, optionally substituted: C₄alkylene, C₃alkylene, C₂alkylene (e.g., —CH₂CH₂—), C₁alkylene (e.g., —CH₂—), and the like.

In some embodiments, L is substituted C₁-C₄alkylene, wherein the C₁-C₄alkylene is substituted with deuterium, oxo, halo, NH₂, NH(C₁-C₄alkyl), N(C₁-C₄alkyl)₂, OH, C₁-C₃alkoxy, NR^{h}CO(C₁-C₄alkyl), NR^{h}CO₂(C₁-C₄alkyl), or an optionally substituted 4-, 5-, or 6-membered heterocycloalkyl containing a nitrogen wherein the nitrogen is connected to the alkylene carbon and each R^{h} is independently hydrogen or C₁-C₄alkyl.

In further embodiments, L is substituted C₁-C₄alkylene, wherein the C₁-C₄alkylene is substituted with deuterium, C₁-C₄alkyl, oxo, halo, NH₂, OH, C₁-C₃alkoxy, NR^{h}CO(C₁-C₄alkyl), NR^{h}C(═O)O(C₁-C₄alkyl), or an optionally substituted 4-, 5-, or 6-membered heterocycloalkyl containing a nitrogen wherein the nitrogen is connected to the alkylene carbon and each R^{h} is independently hydrogen or C₁-C₄alkyl.

In some embodiments in which L is substituted C₁-C₄alkylene, the C₁-C₄alkylene is substituted with C₁-C₄alkyl, OH, NH₂, NH(C₁-C₄alkyl), N(C₁-C₄alkyl)₂, or halo.

In other embodiments in which L is substituted C₁-C₄alkylene, the C₁-C₄alkylene is substituted with CH₃, OH, NH₂, or F.

In further embodiments in which L is substituted C₁-C₄alkylene, the C₁-C₄alkylene is substituted with OH, NH₂, oxo, F, or OCH₃.

In yet further embodiments in which L is substituted C₁-C₄alkylene, the C₁-C₄alkylene is substituted with OH, NH₂, oxo, F, OCH₃, NHCOCH₃, or NHCO₂CH₃.

In some embodiments, L is —CHOH—, —CHNH₂—, —C(═O)—, —CHF—, —CF₂—, or —CH(OCH₃)—.

In further embodiments, L is —CHOH—, —CHNH₂—, —C(═O)—, —CHF—, —CF₂—, —CH(OCH₃)—, —CH(NHCOCH₃)—, or —CHNH(CO₂CH₃)—.

In other embodiments in which L is substituted C₁-C₄alkylene, the C₁-C₄alkylene is substituted with deuterium.

In some embodiments, L is —CHD- or —CD₂-.

In yet other embodiments in which L is substituted C₁-C₄alkylene, the C₁-C₄alkylene is substituted with NHCOCH₃, NHC(═O)OCH₃,

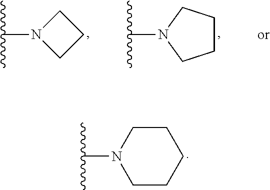

In some embodiments, L is unsubstituted C₁-C₄alkylene, such as, for example, unsubstituted: C₄alkylene, C₃alkylene, C₂alkylene (e.g., —CH₂CH₂—), C₁alkylene (e.g., —CH₂—), and the like. In other embodiments, L is —CH₂—. In further embodiments, L is —CH₂CH₂—.

In some aspects, R² in the compounds of Formula IA, Formula IB, Formula IIA, Formula IIB, Formula IIIA, Formula IIIB, Formula IIIC, Formula IIID, Formula IVA, Formula IVB, Formula VA, Formula VB, Formula VC, or Formula VD is optionally substituted phenyl, optionally substituted pyridinyl, optionally substituted 5-membered heteroaryl, optionally substituted heterocycloalkyl, optionally substituted C₁-C₆alkyl, optionally substituted C₁-C₆alkoxy, optionally substituted C₁-C₆hydroxyalkyl, optionally substituted C₁-C₆haloalkyl, optionally substituted C₁-C₆haloalkoxy, optionally substituted C₃-C₆cycloalkyl, or NR^{f}R^{g}, wherein R^{f} and R^{g} are each independently hydrogen, C₁-C₆alkyl, C₁-C₆haloalkyl, OH, C₁-C₄alkoxy, C₁-C₆hydroxyalkyl, or C₃-C₆cycloalkyl.

In some embodiments, R² in the compounds of Formula IA, Formula IB, Formula IIA, Formula IIB, Formula IIIA, Formula IIIB, Formula IIIC, Formula IIID, Formula IVA, Formula IVB, Formula VA, Formula VB, Formula VC, or Formula VD is optionally substituted phenyl or optionally substituted pyridinyl.

In some embodiments, R² in the compounds of Formula IA, Formula IB, Formula IIA, Formula IIB, Formula IIIA, Formula IIIB, Formula IIIC, Formula IIID, Formula IVA, Formula IVB, Formula VA, Formula VB, Formula VC, or Formula VD is optionally substituted phenyl.

In some embodiments, R² is an unsubstituted phenyl.

In other embodiments, R² is a substituted phenyl. In such embodiments, the phenyl may be substituted with 1, 2, or 3, independently selected substituents.

In some embodiments, R² is a phenyl substituted one or more of halo, C₁-C₄alkyl, C₃-C₄cycloalkyl, C₁-C₄alkoxy, C₁-C₄haloalkoxy, cyano, OH, NR^{b}R^{c}, C(O)NR^{b}R^{c}, CO₂(C₁-C₄alkyl), COOH, C₁-C₄alkylene-R²⁹, or O—C₁-C₄alkylene-R³⁰; R²⁹ is cyano, NR^{b}R^{c}, or COOH; R³⁰ is OH, OCH₃, or NR^{b}R^{c}; and each R^{b} and each R^{c} is independently hydrogen or C₁-C₄alkyl.

In some embodiments, R² is a phenyl substituted with one or more deuterium, halo, cyano, OH, CO₂H, C₁-C₆alkyl, C₁-C₆haloalkyl, C₁-C₆alkoxy, C₁-C₆haloalkoxy, or —C₁-C₄alkyleneCO₂H.

In further embodiments, R² is a phenyl substituted with halo (e.g., F, Cl, Br, I), CO₂H, C₁-C₆alkyl, C₁-C₆haloalkyl, or —C₁-C₄alkyleneCO₂H.

In other embodiments, R² is a phenyl substituted with halo (e.g., F, Cl, Br, I), C₁-C₆alkyl, or C₁-C₆haloalkyl.

In some embodiments, R² is a phenyl substituted with halo (e.g., F, Cl, Br, I), CO₂H, or —C₁-C₄alkyleneCO₂H.

In some embodiments, R² is a phenyl substituted with halo.

In some embodiments, R² is

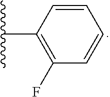 [SMILES: *c1ccccc1F]

In some embodiments, R² is

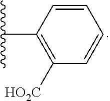 [SMILES: *c1ccccc1C(=O)O]

In some embodiments, R² is

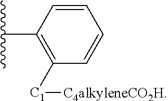

In further embodiments, R² is

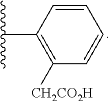

In yet other embodiments, R² is

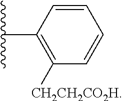

In some embodiments, R² is a phenyl substituted with OH, C₁-C₄alkoxy, or C₁-C₄haloalkoxy. In other embodiments, R² is a phenyl substituted with OH, OCH₃, OCF₃, OCHF₂, or OCH₂F.

In yet other embodiments, R² is

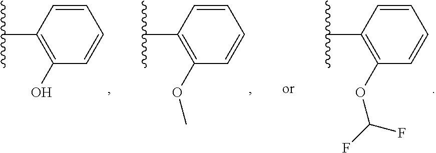

In some embodiments, R² is a phenyl substituted with —C₁-C₄alkyleneNH₂, —C₁-C₄alkyleneNH(C₁-C₆alkylene), or —C₁-C₄alkyleneN(C₁-C₆alkylene)₂. In other embodiments, R² is a phenyl substituted with CH₂NH₂, CH₂NH(CH₃), or CH₂N(CH₃)₂.

In some embodiments, R² is

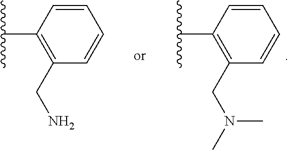 [SMILES: *c1ccccc1CN ; *c1ccccc1CN(C)C]

In some embodiments, R² is a phenyl substituted with cyano or —C₁-C₄alkylene-cyano. In other embodiments, R² is a phenyl substituted with CH₂CN.

In some embodiments, R² is

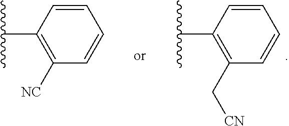 [SMILES: *c1ccccc1C#N ; *c1ccccc1CC#N]

In some embodiments, R² is a phenyl substituted with cyano.

In some embodiments, R² is

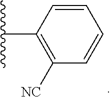 [SMILES: *c1ccccc1C#N]

In some embodiments, R² is a phenyl substituted one or more of O—C₁-C₄alkylene-R³⁰; wherein R³⁰ is OH, OCH₃, or NR^{b}R^{c}; and each R^{b} and each R^{c} is independently hydrogen or C₁-C₄alkyl. In other embodiments, R² is a phenyl substituted with OCH₂CH₂OH or OCH₂CH₂OCH₃.

In some embodiments, R² is

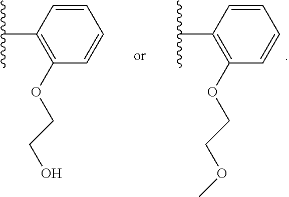 [SMILES: *c1ccccc1OCCO ; *c1ccccc1OCCOC]

In some embodiments, R² is a phenyl substituted with one halo and one cyano, OH, or C₁-C₄alkoxy. In some embodiments, R² is

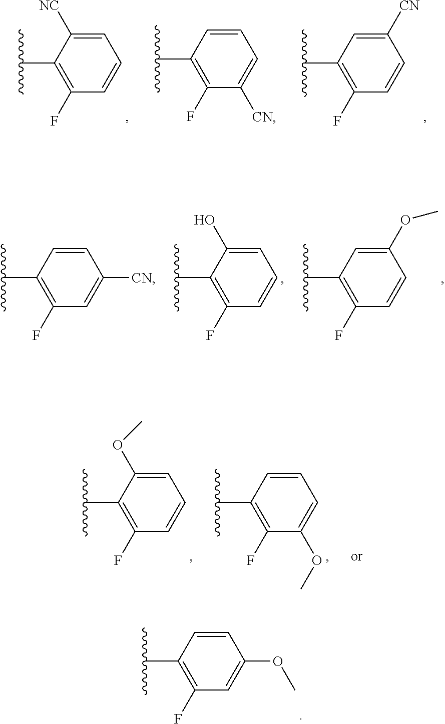

In some embodiments, R² is a phenyl substituted with one halo and one cyano. In some embodiments, R² is

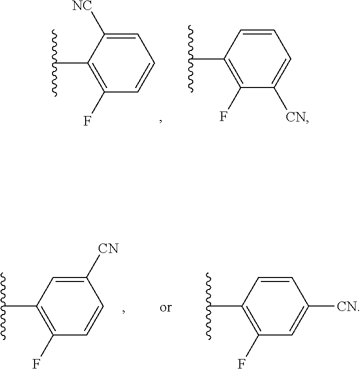

In some embodiments, R² is

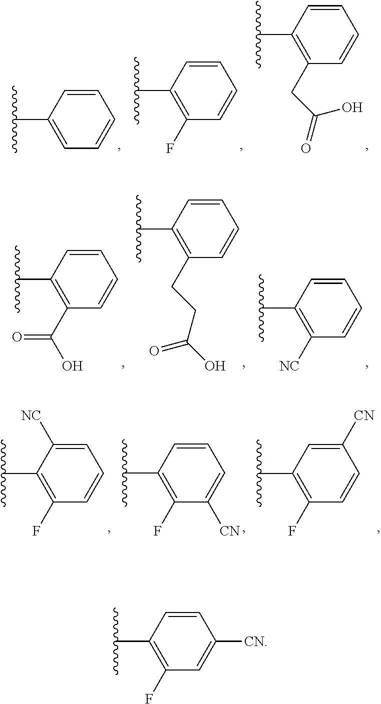

In yet other embodiments, R² is

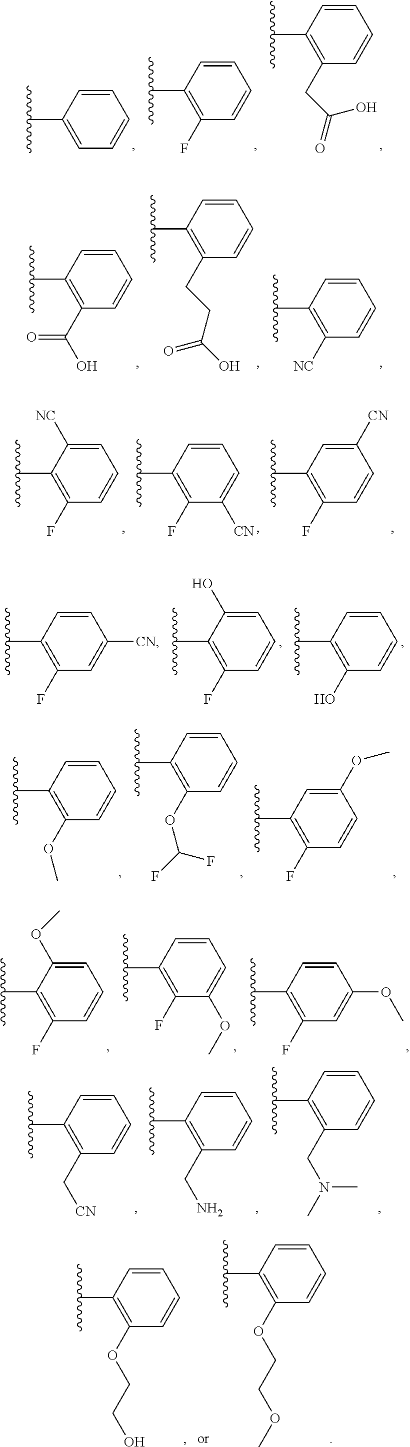

In some embodiments, R² is phenyl substituted with 1, 2, 3, 4, or 5 deuterium atoms. In other embodiments, the phenyl with 1, 2, 3, or 4 deuterium atoms may be additionally substituted with one or more halo, C₁-C₆alkyl, cyano, or C₁-C₆haloalkyl. In yet further embodiments, R² is

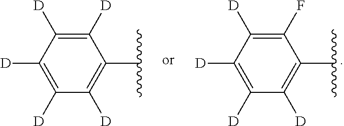 [SMILES: *c1c([2H])c([2H])c([2H])c([2H])c1[2H] ; *c1c([2H])c([2H])c([2H])c([2H])c1F]

In some embodiments, R² in the compounds of Formula IA, Formula IB, Formula IIA, Formula IIB, Formula IIIA, Formula IIIB, Formula IIIC, Formula IIID, Formula IVA, Formula IVB, Formula VA, Formula VB, Formula VC, or Formula VD is optionally substituted pyridinyl. In such embodiments, the optionally substituted pyridinyl may attach to L at any carbon atom in the pyridinyl ring, i.e.,

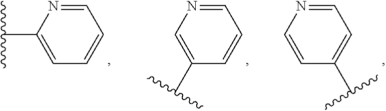

and the like, wherein

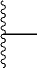

indicates the point of attachment to L.

In some embodiments, R² is an unsubstituted pyridinyl.

In some embodiments, R² is

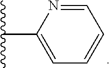 [SMILES: *c1ccccn1]

In other embodiments, R² is a substituted pyridinyl. In such embodiments, the pyridinyl may be substituted with 1, 2, or 3, independently selected substituents.

In some embodiments, R² is a pyridinyl substituted one or more of halo, C₁-C₄alkyl, C₃-C₄cycloalkyl, C₁-C₄alkoxy, C₁-C₄haloalkoxy, cyano, OH, NR^{b}R^{c}, C(O)NR^{b}R^{c}, CO₂(C₁-C₄alkyl), COOH, C₁-C₄alkylene-R²⁹, or O—C₁-C₄alkylene-R³⁰; R²⁹ is cyano, NR^{b}R^{c}, or COOH; R³⁰ is OH, OCH₃, or NR^{b}R^{c}; and each R^{b} and each R^{c} is independently hydrogen or C₁-C₄alkyl.

In some embodiments, the pyridinyl nitrogen atom is substituted by an oxide (═O) to form an N-oxide, such as, for example,

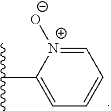 [SMILES: *c1cccc[n+]1[O-]]

In some embodiments, R² is a pyridinyl substituted with one or more one or more deuterium, halo, cyano, OH, CO₂H, C₁-C₆alkyl, C₁-C₆haloalkyl, C₁-C₆alkoxy, C₁-C₆haloalkoxy, or —C₁-C₄alkyleneCO₂H.

In further embodiments, R² is a pyridinyl substituted with halo (e.g., F, Cl, Br, I), CO₂H, C₁-C₆alkyl, C₁-C₆haloalkyl, or —C₁-C₄alkyleneCO₂H.

In other embodiments, R² is a pyridinyl substituted with halo (e.g., F, Cl, Br, I), C₁-C₆alkyl, or C₁-C₆haloalkyl.

In some embodiments, R² is a pyridinyl substituted with halo (e.g., F, Cl, Br, I), CO₂H, or —C₁-C₄alkyleneCO₂H.

In other embodiments, R² is a pyridinyl substituted with halo or C₁-C₄alkyl.

In some embodiments, R² is a pyridinyl substituted with halo (e.g., —F, —Cl, —Br, —I).

In further embodiments, R² is

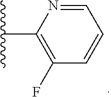 [SMILES: *c1ncccc1F]

In some embodiments, R² is a pyridinyl substituted with C₁-C₄alkyl.

In other embodiments, R² is

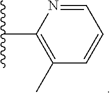 [SMILES: *c1ncccc1C]

In further embodiments, R² is

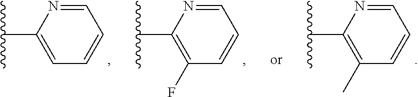

In other embodiments, R² is pyridinyl substituted with 1, 2, 3, or 4 deuterium atoms. In further embodiments, the pyridinyl with 1, 2, or 3 deuterium atoms may be additionally substituted with one or more halo, C₁-C₆alkyl, or C₁-C₆haloalkyl. In yet other embodiments, R² is

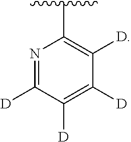

In some embodiments, R² in the compounds of Formula IA, Formula IB, Formula IIA, Formula IIB, Formula IIIA, Formula IIIB, Formula IIIC, Formula IIID, Formula IVA, Formula IVB, Formula VA, Formula VB, Formula VC, or Formula VD is optionally substituted 5-membered heteroaryl, such as, for example, optionally substituted: pyrrolyl, pyrazolyl, imidazolyl, triazolyl, tetrazolyl, oxadiazolyl, oxazolyl, isoxazolyl, isothiazolyl, thiazolyl, or thiadiazolyl. In such embodiments, the optionally substituted 5-membered heteroaryl may attach to L at a carbon atom or a nitrogen atom in the 5-membered heteroaryl ring.

In other embodiments, R² in the compounds of Formula IA, Formula IB, Formula IIA, Formula IIB, Formula IIIA, Formula IIIB, Formula IIIC, Formula IIID, Formula IVA, Formula IVB, Formula VA, Formula VB, Formula VC, or Formula VD is optionally substituted; triazolyl, pyrazolyl, oxazolyl, or isoxazolyl.

In further embodiments, R² is an unsubstituted 5-membered heteroaryl.

In some embodiments, R² is an unsubstituted pyrazolyl.

In some embodiments, R² is

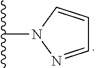 [SMILES: *n1cccn1]

In other embodiments, R² is

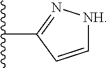 [SMILES: *c1cc[nH]n1]

In some embodiments, R² is an unsubstituted triazolyl.

In some embodiments, R² is

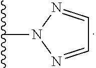 [SMILES: *n1nccn1]

In some embodiments, R² is a substituted 5-membered heteroaryl. In such embodiments, the 5-membered heteroaryl may be substituted with 1, 2, or 3, independently selected substituents.

In some embodiments, R² is a 5-membered heteroaryl substituted with one or more halo (e.g., —F, —Cl, —Br, —I), CO₂H, C₁-C₆alkyl, C₁-C₆haloalkyl, or —C₁-C₄alkyleneCO₂H.

In other embodiments, R² is a 5-membered heteroaryl substituted with one or more halo (e.g., —F, —Cl, —Br, —I), C₁-C₆alkyl, or C₁-C₆haloalkyl.

In further embodiments, R² is a 5-membered heteroaryl substituted with one or more C₁-C₆alkyl. In some embodiments, R² is a 5-membered heteroaryl substituted with one or more CH₃. In some embodiments, R² is

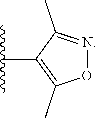 [SMILES: *c1c(C)noc1C]

In other embodiments, R² is

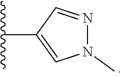 [SMILES: *c1cnn(C)c1]

In further embodiments, R² is

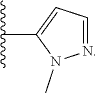 [SMILES: *c1ccnn1C]

In yet other embodiments, R² is

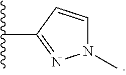 [SMILES: *c1ccn(C)n1]

In other embodiments, R² is

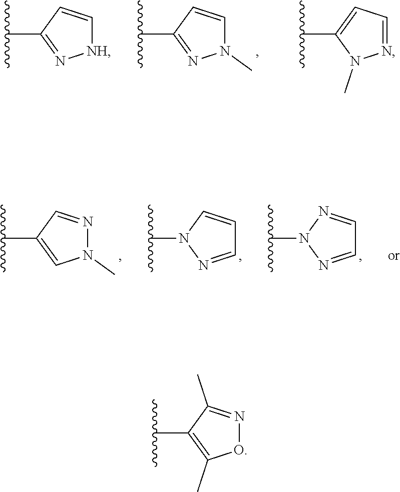

In some embodiments, R² in the compounds of Formula IA, Formula IB, Formula IIA, Formula IIB, Formula IIIA, Formula IIIB, Formula IIIC, Formula IIID, Formula IVA, Formula IVB, Formula VA, Formula VB, Formula VC, or Formula VD is optionally substituted heterocycloalkyl, such as, for example, optionally substituted: azetidinyl, pyrrolidinyl, piperidinyl, morpholinyl, dioxanyl, tetrahydropyranyl, thianyl, thianyl sulfone, thianyl sulfoxide, or 1-oxo-1-imino-1-thiacyclohexyl. In such embodiments, the optionally substituted heterocycloalkyl may attach to L at a carbon atom or a nitrogen atom in the heterocycloalkyl ring.

In other embodiments, R² is optionally substituted azetidinyl, optionally substituted pyrrolidinyl, optionally substituted tetrahydropyranyl, optionally substituted piperidinyl, optionally substituted morpholinyl, or optionally substituted dioxanyl.

In further embodiments, R² is optionally substituted optionally substituted piperidinyl, optionally substituted morpholinyl, optionally substituted dioxanyl, or optionally substituted tetrahydropyranyl.

In some embodiments, R² is optionally substituted piperidinyl or tetrahydropyranyl.

In some embodiments, R² in the compounds of Formula IA, Formula IB, Formula IIA, Formula IIB, Formula IIIA, Formula IIIB, Formula IIIC, Formula IIID, Formula IVA, Formula IVB, Formula VA, Formula VB, Formula VC, or Formula VD is an unsubstituted heterocycloalkyl.

In some embodiments, R² is

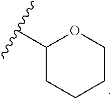 [SMILES: *C1CCCCO1]

In other embodiments, R² is

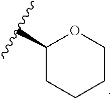 [SMILES: *[C@@H]1CCCCO1]

In further embodiments, R² is

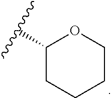 [SMILES: *[C@H]1CCCCO1]

In some embodiments, R² is

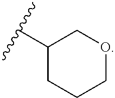 [SMILES: *C1CCCOC1]

In other embodiments, R² is

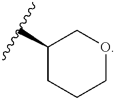 [SMILES: *[C@@H]1CCCOC1]

In further embodiments, R² is

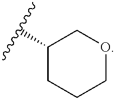 [SMILES: *[C@H]1CCCOC1]

In some embodiments, R² is

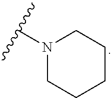 [SMILES: *N1CCCCC1]

In some embodiments, R² is

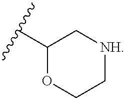 [SMILES: *C1CNCCO1]

In some embodiments, R² is

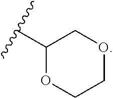 [SMILES: *C1COCCO1]

In other embodiments, R² is

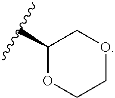 [SMILES: *[C@H]1COCCO1]

In further embodiments, R² is

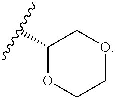 [SMILES: *[C@@H]1COCCO1]

In some embodiments, R² is

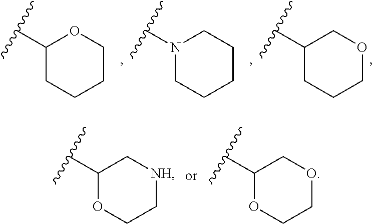

In some embodiments, R² is a substituted heterocycloalkyl. In such embodiments, the heterocycloalkyl may be substituted with 1, 2, or 3, independently selected substituents.

In some embodiments, R² in the compounds of Formula IA, Formula IB, Formula IIA, Formula IIB, Formula IIIA, Formula IIIB, Formula IIIC, Formula IIID, Formula IVA, Formula IVB, Formula VA, Formula VB, Formula VC, or Formula VD is optionally substituted C₁-C₆alkyl, such as, for example, optionally substituted: C₆alkyl, C₅alkyl, C₄alkyl such as CH₂CH(CH₃)₂, C₃alkyl such as CH(CH₃)₂, C₂alkyl, or C₁alkyl (e.g., CH₃).

In some embodiments, R² is unsubstituted C₁-C₆alkyl. In other embodiments, R² is CH₃, CH₂CH₃, CH(CH₃)₂, CH₂CH(CH₃)₂, C(CH₃)₃, or CH₂C(CH₃)₃. In further embodiments, R² is CH₃. In other embodiments, R² is CH₂CH₃. In yet other embodiments, R² is CH₂CH(CH₃)₂. In some embodiments, R² is CH(CH₃)₂. In other embodiments, R² is C(CH₃)₃. In yet other embodiments, R² is CH₂C(CH₃)₃.

In some embodiments, R² is C₁-C₆alkyl substituted with COOH. In other embodiments, R² is CH₂C(CH₃)₂(COOH).

In some embodiments, R² in the compounds of Formula IA, Formula IB, Formula IIA, Formula IIB, Formula IIIA, Formula IIIB, Formula IIIC, Formula IIID, Formula IVA, Formula IVB, Formula VA, Formula VB, Formula VC, or Formula VD is optionally substituted C₁-C₆alkoxy, such as, for example, optionally substituted: C₆alkoxy, C₅alkoxy, C₄alkoxy, C₃alkoxy such as OCH(CH₃)₂, C₂alkoxy, or C₁alkoxy (e.g., OCH₃).

In some embodiments, R² is unsubstituted C₁-C₆alkoxy. In other embodiments, R² is OCH₃ or OCH(CH₃)₂. In yet other embodiments, R² is OCH₃. In further embodiments, R² is OCH(CH₃)₂.

In some embodiments, R² in the compounds of Formula IA, Formula IB, Formula IIA, Formula IIB, Formula IIIA, Formula IIIB, Formula IIIC, Formula IIID, Formula IVA, Formula IVB, Formula VA, Formula VB, Formula VC, or Formula VD is optionally substituted C₁-C₆hydroxyalkyl, such as, for example, optionally substituted: C₆hydroxyalkyl, C₅hydroxyalkyl, C₄hydroxyalkyl, C₃hydroxyalkyl such as C(OH)(CH₃)₂, C₂hydroxyalkyl such as CH(OH)(CH₃), or C₁hydroxyalkyl.

In some embodiments, R² is unsubstituted C₁-C₆hydroxyalkyl. In other embodiments, R² is C(OH)(CH₃)₂ or CH(OH)(CH₃).

In some embodiments, R² in the compounds of Formula IA, Formula IB, Formula IIA, Formula IIB, Formula IIIA, Formula IIIB, Formula IIIC, Formula IIID, Formula IVA, Formula IVB, Formula VA, Formula VB, Formula VC, or Formula VD is optionally substituted C₁-C₆haloalkyl, such as, for example, optionally substituted: C₆haloalkyl, C₅haloalkyl, C₄haloalkyl, C₃haloalkyl such as CH(CH₃)(CF₃), C₂haloalkyl such as CH₂CF₃, or C₁haloalkyl such as CF₃, CHF₂ or CH₂F.

In some embodiments, R² is unsubstituted C₁-C₆haloalkyl. In other embodiments, R² is CF₃, CH(CH₃)(CF₃), CH₂CF₃, C(CH₃)₂F, CH₂C(CH₃)₂F, C(CH₃)F₂, CH₂C(CH₃)F₂, CHFCH(CH₃)₂, or CF₂CH(CH₃)₂. In further embodiments, R² is CF₃. In other embodiments, R² is CH₂CF₃. In yet other embodiments, R² is CH(CH₃)(CF₃). In further embodiments, R² is C(CH₃)₂F. In other embodiments, R² is CH₂C(CH₃)₂F. In yet other embodiments, R² is C(CH₃)F₂. In further embodiments, R² is CH₂C(CH₃)F₂. In other embodiments, R² is CHFCH(CH₃)₂. In yet other embodiments, R² is CF₂CH(CH₃)₂.

In some embodiments, R² in the compounds of Formula IA, Formula IB, Formula IIA, Formula IIB, Formula IIIA, Formula IIIB, Formula IIIC, Formula IIID, Formula IVA, Formula IVB, Formula VA, Formula VB, Formula VC, or Formula VD is optionally substituted: C₁-C₆haloalkoxy, such as, for example, C₆haloalkoxy, C₅haloalkoxy, C₄haloalkoxy, C₃haloalkoxy such as OCH(CH₃)(CF₃), C₂haloalkoxy such as OCH₂CF₃, or C₁haloalkoxy such as OCF₃, OCHF₂, or OCH₂F.

In some embodiments, R² is unsubstituted C₁-C₆haloalkoxy. In other embodiments, R² is OCF₃, OCH₂CF₃, OCHF₂, OCH₂F, or OCH(CH₃)(CF₃). In some embodiments, R² is OCF₃. In other embodiments, R² is OCH₂CF₃. In yet other embodiments, R² is OCHF₂. In further embodiments, R² is OCH₂F. In other embodiments, R² is OCH(CH₃)(CF₃).

In some embodiments, R² in the compounds of Formula IA, Formula IB, Formula IIA, Formula IIB, Formula IIIA, Formula IIIB, Formula IIIC, Formula IIID, Formula IVA, Formula IVB, Formula VA, Formula VB, Formula VC, or Formula VD is optionally substituted C₃-C₆cycloalkyl.

In some embodiments, R² is unsubstituted C₃-C₆cycloalkyl.

In other embodiments, R² is C₃-C₆cycloalkyl, optionally substituted with one or more of C₁-C₄alkyl or C₁-C₄haloalkyl, In further embodiments, R² is cyclopropyl, cyclobutyl,

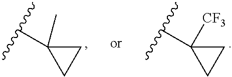

In some embodiments, R² in the compounds of Formula IA, Formula IB, Formula IIA, Formula IIB, Formula IIIA, Formula IIIB, Formula IIIC, Formula IIID, Formula IVA, Formula IVB, Formula VA, Formula VB, Formula VC, or Formula VD is NR^{f}R^{g}, wherein R^{f} and R^{g} are each independently hydrogen, C₁-C₆alkyl, C₁-C₆haloalkyl, OH, C₁-C₄alkoxy, C₁-C₆hydroxyalkyl, or C₃-C₆cycloalkyl.

In some embodiments, R² is NH₂, N(H)(CH₃), N(CH₃)₂, NH(CH(CH₃)₂), N(CH₃)(CH₂CH₃), or N(CH₃)(cyclopropyl).

In some aspects, Ar¹ in the compounds of Formula IA, Formula IB, Formula IIA, Formula IIB, Formula IIIA, Formula IIIB, Formula IIIC, Formula IIID, Formula IVA, Formula IVB, Formula VA, Formula VB, Formula VC, or Formula VD is an optionally substituted five-membered heteroaryl, such as, for example, optionally substituted: pyrrolyl, pyrazolyl, imidazolyl, triazolyl, tetrazolyl, oxadiazolyl, oxazolyl, isoxazolyl, isothiazolyl, thiazolyl, thiadiazolyl, or thiophenyl. In such embodiments, the optionally substituted five-membered heteroaryl may attach to the amide C(O) group at a carbon atom or a nitrogen atom in the heterocycloalkyl ring.

In some embodiments, Ar¹ is an optionally substituted thiazolyl or optionally substituted thiophenyl.

In some embodiments, Ar¹ is a substituted five-membered heteroaryl, such as, for example, a substituted pyrrolyl, pyrazolyl, imidazolyl, triazolyl, tetrazolyl, oxadiazolyl, oxazolyl, isoxazolyl, isothiazolyl, thiazolyl, thiadiazolyl, or thiophenyl. In such embodiments, the 5-membered heteroaryl may be substituted with 1, 2, or 3, independently selected substituents.

In some embodiments, the five-membered heteroaryl is substituted with one or more halo, C₁-C₆alkyl, C₁-C₆haloalkyl, optionally substituted C₂-C₄alkenyl, optionally substituted C₂-C₄alkynyl, C₃-C₆cycloalkyl, or cyano. In other embodiments, the C₂-C₄alkenyl and C₂-C₄alkynyl are unsubstituted.

In other embodiments, the five-membered heteroaryl is substituted with one or more halo, cyano, C≡CH, CH═CH₂, C₁-C₃alkyl, or C₁-C₃haloalkyl.

In some embodiments, the five-membered heteroaryl is substituted with halo (—F, —Cl, —Br, or —I), cyano, C≡CH, C₁-C₃alkyl, or C₁-C₃haloalkyl.

In some embodiments, the five-membered heteroaryl is substituted with halo, cyano, CHF₂, or CF₃.

In other embodiments, the five-membered heteroaryl is substituted with Br, Cl, cyano, C≡CH, CH═CH₂, CHF₂, or CF₃.

In some embodiments, Ar¹ is an unsubstituted five-membered heteroaryl, such as, for example, an unsubstituted pyrrolyl, pyrazolyl, imidazolyl, triazolyl, tetrazolyl, oxadiazolyl, oxazolyl, isoxazolyl, isothiazolyl, thiazolyl, thiadiazolyl, or thiophenyl.

In some embodiment, Ar¹ in the compounds of Formula IA, Formula IB, Formula IIA, Formula IIB, Formula IIIA, Formula IIIB, Formula IIIC, Formula IIID, Formula IVA, Formula IVB, Formula VA, Formula VB, Formula VC, or Formula VD is

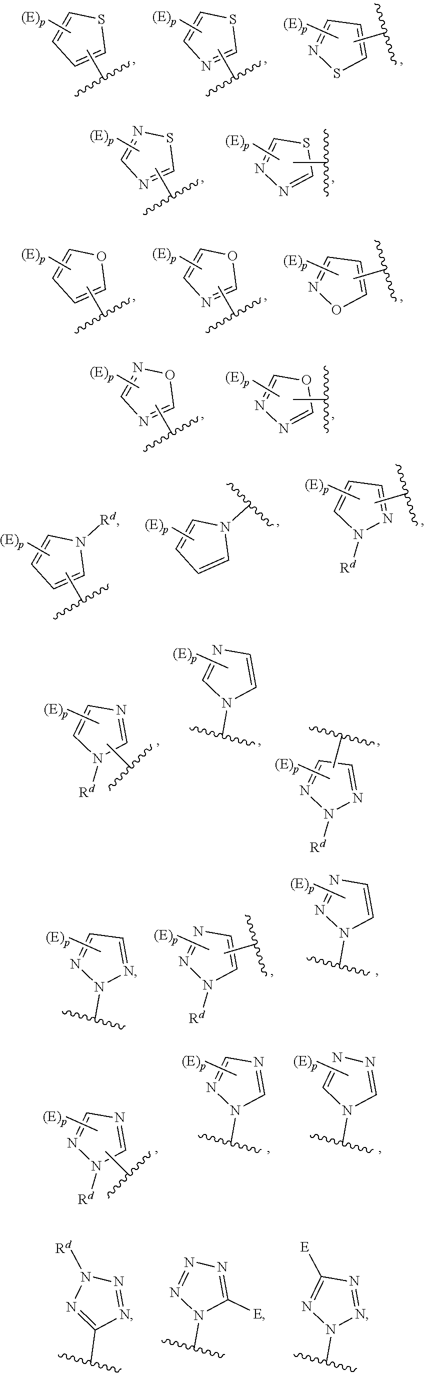

wherein:
each instance of R^{d} is independently hydrogen, optionally substituted C₁-C₆alkyl, or a nitrogen protecting group;
each instance of E is independently halo, CN, NO₂, N₃, an optionally substituted alkyl, an optionally substituted alkenyl, an optionally substituted alkynyl, an optionally substituted cycloalkyl, an optionally substituted aryl, an optionally substituted heterocycloalkyl, an optionally substituted heteroaryl, OR^{o1}, SR^{s1}, N(Rⁿ¹)₂, C(═O)N(Rⁿ¹)₂, N(Rⁿ¹)C(═O)R^{c1}, C(═O)R^{o1}, C(═O)OR^{o1}, OC(═O)R^{o1}, S(═O)R^{s1}, S(═O)₂R^{s1}, S(═O)(C₃-C₆cycloalkyl), S(═O)₂(C₃-C₆cycloalkyl), S(═O)OR^{o1}, OS(═O)R^{c1}, S(═O)₂OR^{o1}, OS(═O)₂R^{c1}, S(═O)N(Rⁿ¹)₂, S(═O)₂N(Rⁿ¹)₂, N(Rⁿ¹)S(═O)R^{s1}, N(Rⁿ¹)S(═O)₂R^{s1}, N(Rⁿ¹)C(═O)OR^{o1}, OC(═O)N(Rⁿ¹)₂, N(Rⁿ¹)C(═O)N(Rⁿ¹)₂, N(Rⁿ¹)S(═O)N(Rⁿ¹)₂, N(Rⁿ¹)S(═O)₂N(Rⁿ¹)₂, N(Rⁿ¹)S(═O)OR^{o1}, N(Rⁿ¹)S(═O)₂OR^{o1}, OS(═O)N(Rⁿ¹)₂, or OS(═O)₂N(Rⁿ¹)₂; or alternatively two instances of (E)ₚ attached to adjacent carbon atoms, can be taken together with the carbon atom(s) to which they are attached to form an optionally substituted cycloalkyl or a heterocycloalkyl; wherein:
   each instance of Rⁿ¹ is independently hydrogen, an optionally substituted C₁-C₆alkyl, or a nitrogen protecting group;
   each instance of R^{o1} is independently hydrogen, an optionally substituted C₁-C₆alkyl, or an oxygen protecting group; and
   each instance of R^{c1} is independently an optionally substituted C₁-C₆alkyl; and
   each instance of R^{s1} is independently an optionally substituted C₁-C₆alkyl or a sulfur protecting group; and
   p is 0, 1, 2, 3, or 4, as valency permits. Where a particular atom is not substituted with E, it should be understood as having the required number of hydrogens as valency permits so as to form a stable ring.

In some embodiments, Ar¹ is

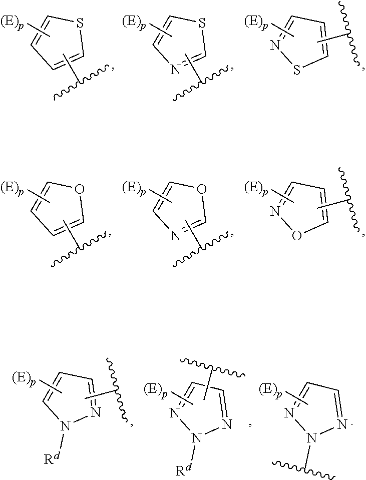

In some embodiments, Ar¹ is

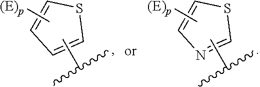

In some embodiments, Ar¹ is

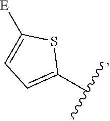

wherein E is halo, cyano, C₁-C₆alkyl, C₁-C₆haloalkyl, optionally substituted C₂-C₄alkenyl, optionally substituted C₂-C₄alkynyl, or C₃-C₆cycloalkyl. In other embodiments, E is CHF₂, CF₃, or CH₂CHF₂. In some embodiments, the C₂-C₄alkenyl and C₂-C₄alkynyl groups are unsubstituted.

In some embodiments, Ar¹ is

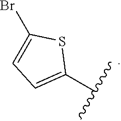 [SMILES: *c1ccc(Br)s1]

In some embodiments, Ar¹ is

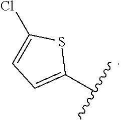 [SMILES: *c1ccc(Cl)s1]

In some embodiments, Ar¹ is

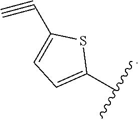 [SMILES: *c1ccc(C#C)s1]

In some embodiments, Ar¹ is

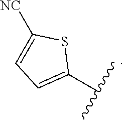 [SMILES: *c1ccc(C#N)s1]

In some embodiments, Ar¹ is

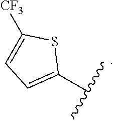 [SMILES: *c1ccc(C(F)(F)F)s1]

In some embodiments, Ar¹ is

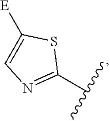

wherein E is halo, cyano, C₁-C₆alkyl, C₁-C₆haloalkyl, optionally substituted C₂-C₄alkenyl, optionally substituted C₂-C₄alkynyl, or C₃-C₆cycloalkyl. In other embodiments E is CHF₂, CF₃, or CH₂CHF₂. In some embodiments, the C₂-C₄alkenyl and C₂-C₄alkynyl groups are unsubstituted.

In some embodiments, Ar¹ is

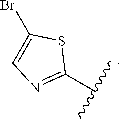 [SMILES: *c1ncc(Br)s1]

In some embodiments, Ar¹ is

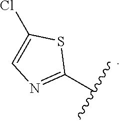 [SMILES: *c1ncc(Cl)s1]

In some embodiments, Ar¹ is

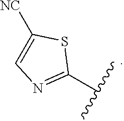 [SMILES: *c1ncc(C#N)s1]

In some embodiments, Ar¹ is

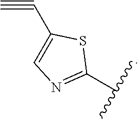 [SMILES: *c1ncc(C#C)s1]

In some embodiments, Ar¹ is

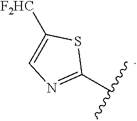 [SMILES: *c1ncc(C(F)F)s1]

In some embodiments, Ar¹ is

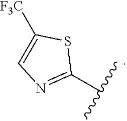 [SMILES: *c1ncc(C(F)(F)F)s1]

In some embodiments, Ar¹ is

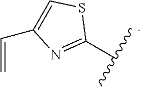 [SMILES: *c1nc(C=C)cs1]

In some aspects, L² in the compounds of Formula IIA or compounds of Formula IIB is —O—, —NR^{a}—, or —CH₂—, wherein R^{a} is hydrogen or C₁-C₄alkyl.

In some embodiments, L² is —O—. In other embodiments, L² is —NR^{a}—. In yet other embodiments, L² is —NH—. In further embodiments, L² is —CH₂—.

In some aspects, Ar² in the compounds of Formula IIA or compounds of Formula IIB is optionally substituted heteroaryl, optionally substituted aryl, or optionally substituted 4-, 5- or 6-membered heterocycloalkyl.

In some embodiments, Ar² in the compounds of Formula IIA or compounds of Formula IIB is optionally substituted 5- or 6-membered heteroaryl or optionally substituted 4-, 5-, or 6-membered heterocycloalkyl.

In some embodiments, Ar² in the compounds of Formula IIA or compounds of Formula IIB is optionally substituted 5- or 6-membered heteroaryl, such as, for example, optionally substituted: pyrrolyl, pyrazolyl, imidazolyl, triazolyl, tetrazolyl, oxadiazolyl, oxazolyl, isoxazolyl, isothiazolyl, thiazolyl, thiadiazolyl, pyridinyl, pyrimidinyl, pyrazinyl, or pyridazinyl.

In some embodiments, Ar² in the compounds of Formula IIA or compounds of Formula IIB is an unsubstituted 5- or 6-membered heteroaryl, such as, for example, an unsubstituted: pyrrolyl, pyrazolyl, imidazolyl, triazolyl, tetrazolyl, oxadiazolyl, oxazolyl, isoxazolyl, isothiazolyl, thiazolyl, thiadiazolyl, pyridinyl, pyrimidinyl, pyrazinyl, or pyridazinyl.

In other embodiments, Ar² in the compounds of Formula IIA or compounds of Formula IIB is a substituted 5- or 6-membered heteroaryl, such as, for example, a substituted: pyrrolyl, pyrazolyl, imidazolyl, triazolyl, tetrazolyl, oxadiazolyl, oxazolyl, isoxazolyl, isothiazolyl, thiazolyl, thiadiazolyl, pyridinyl, pyrimidinyl, pyrazinyl, or pyridazinyl. In some embodiments, the 5- or 6-membered heteroaryl is substituted with one or more NR^{b}R^{c}(wherein each R^{b} and R^{c} is independently hydrogen or C₁-C₄alkyl), halo, oxo, cyano, C₁-C₄alkyl, C₁-C₄haloalkyl, C₁-C₄alkoxy, C₁-C₆hydroxyalkyl, or —C(O)OC₁-C₄alkyl.

In some embodiments, Ar² in the compounds of Formula IIA or compounds of Formula IIB is substituted pyrazolyl.

In some embodiments, Ar² in the compounds of Formula IIA or compounds of Formula IIB is pyrazolyl substituted with one or more C₁-C₄alkyl, C₁-C₄haloalkyl, halo, or NR^{b}R^{c} wherein each R^{b} and R^{c} is independently hydrogen or C₁-C₄alkyl.

In some embodiments, Ar² in the compounds of Formula IIA or compounds of Formula IIB is pyrazolyl substituted with a C₁-C₄alkyl group. In other embodiments, Ar² in the compounds of Formula IIA or compounds of Formula IIB is pyrazolyl substituted with a C₁-C₃alkyl group. In further embodiments, Ar² in the compounds of Formula IIA or compounds of Formula IIB is pyrazolyl substituted with a methyl group. In some embodiments, Ar² in the compounds of Formula IIA or compounds of Formula IIB is 1-methylpyrazol-3-yl.

In further embodiments, Ar² in the compounds of Formula IIA or compounds of Formula IIB is optionally substituted aryl, such as, for example, optionally substituted phenyl.

In other embodiments, Ar² in the compounds of Formula IIA or compounds of Formula IIB is an unsubstituted aryl, such as, for example, an unsubstituted phenyl.

In other embodiments, Ar² in the compounds of Formula IIA or compounds of Formula IIB is a substituted aryl, such as, for example, a substituted phenyl. In some embodiments, the phenyl is substituted with one or more NR^{b}R^{c}(wherein each R^{b} and R^{c} is independently hydrogen or C₁-C₄alkyl), halo, oxo, cyano, C₁-C₆alkyl, C₁-C₆haloalkyl, C₁-C₆haloalkyl, C₁-C₆alkoxy, C₁-C₆hydroxyalkyl, or —C(O)OC₁-C₄alkyl.

In yet further embodiments, Ar² in the compounds of Formula IIA or compounds of Formula IIB is optionally substituted 4-, 5- or 6-membered heterocycloalkyl, such as, for example, optionally substituted: azetidinyl, pyrrolidinyl, dioxolanyl, imidazolidinyl, pyrazolidinyl, piperazinyl, piperidinyl, dioxanyl, morpholinyl, thianyl, thianyl sulfoxide, 1-oxo-1-imino-1thiacyclohexyl, dithianyl, thiomorpholinyl, oxetanyl, tetrahydrofuranyl, tetrahydropyranyl, or tetrahydrothiophenyl.

In some embodiments, Ar² in the compounds of Formula IIA or compounds of Formula IIB is an optionally substituted 4-membered heterocycloalkyl, such as, for example, optionally substituted: azetidinyl or oxetanyl.

In some embodiments, Ar² in the compounds of Formula IIA or compounds of Formula IIB is an optionally substituted 5-membered heterocycloalkyl. such as, for example, optionally substituted: pyrrolidinyl, dioxolanyl, imidazolidinyl, pyrazolidinyl, tetrahydrofuranyl, or tetrahydrothiophenyl.

In some embodiments, Ar² in the compounds of Formula IIA or compounds of Formula IIB is an optionally substituted 6-membered heterocycloalkyl. such as, for example, optionally substituted: piperazinyl, piperidinyl, dioxanyl, morpholinyl, thianyl, thianyl sulfoxide, 1-oxo-1-imino-1thiacyclohexyl, dithianyl, thiomorpholinyl, or tetrahydropyranyl.

In some embodiments, Ar² in the compounds of Formula IIA or compounds of Formula IIB is

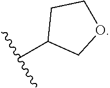 [SMILES: *C1CCOC1]

In other embodiments, Ar² is

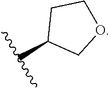 [SMILES: *[C@H]1CCOC1]

In further embodiments, Ar² is

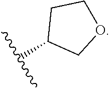 [SMILES: *[C@@H]1CCOC1]

In some embodiments, Ar² in the compounds of Formula IIA or compounds of Formula IIB is optionally substituted tetrahydrofuranyl or optionally substituted pyrazolyl.

In some embodiments, the compounds of Formula IA are compounds of Formula IA-1:

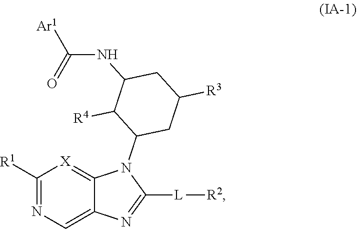

or a pharmaceutically acceptable salt thereof, wherein R³ is hydrogen, OH, C₁-C₃alkoxy, cyano, OSO₂(C₁-C₃alkyl), OSO₂(C₁-C₃alkyl), N₃, NH₂, NH(C₁-C₆alkyl), or N(C₁-C₆alkyl)₂; R⁴ is hydrogen, OH, C₁-C₃alkoxy, cyano, OSO₂(C₁-C₃alkyl), OSO₂(C₁-C₃alkyl), N₃, NH₂, NH(C₁-C₆alkyl), or N(C₁-C₆alkyl)₂, and wherein at least one of R³ and R⁴ is hydrogen, and Ar¹, R¹, R², L, and X are defined herein.

In some embodiments of the compounds of Formula IA, R³ and R⁴ are each independently hydrogen, deuterium, —OH, halo, cyano, C₁-C₃alkyl, C₁-C₃haloalkyl, C₁-C₃alkoxy, C₁-C₃haloalkoxy, —NH₂, —NH(C₁-C₆alkyl), —N(C₁-C₆alkyl)₂, or deuterated C₁-C₃alkoxy.

In some embodiments of the compounds of Formula IA-1, R³ and R⁴ are each independently hydrogen, —OH, halo, cyano, C₁-C₃alkyl, C₁-C₃haloalkyl, C₁-C₃alkoxy, C₁-C₃haloalkoxy, —NH₂, —NH(C₁-C₆alkyl), or —N(C₁-C₆alkyl)₂.

In some embodiments of the compounds of Formula IA-1, at least one of R³ and R⁴ is hydrogen.

In some embodiments of the compounds of Formula IA-1, at least one of R³ and R⁴ is deuterium.

In some embodiments, the compounds of Formula IA-1 are compounds of Formula IA-1a:

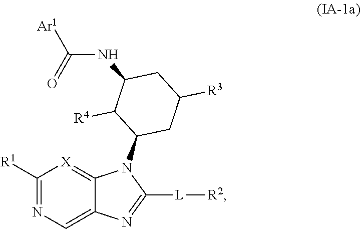

or a pharmaceutically acceptable salt thereof.

In some embodiments of the compounds of Formulas IA-1 and IA-1a, R³ is OH, C₁-C₃alkoxy, cyano, OCO₂(C₁-C₃alkyl), OSO₂(C₁-C₃alkyl), N₃, NH₂, NH(C₁-C₆alkyl), or N(C₁-C₆alkyl)₂; and R⁴ is hydrogen.

In some embodiments of the compounds of Formulas IA-1 and IA-1a, R³ is hydrogen and R⁴ is OH, C₁-C₃alkoxy, cyano, OSO₂(C₁-C₃alkyl), OSO₂(C₁-C₃alkyl), N₃, NH₂, NH(C₁-C₆alkyl), or N(C₁-C₆alkyl)₂.

In some embodiments of the compounds of Formulas IA-1 and IA-1a, R³ is OH or C₁-C₃alkoxy and R⁴ is hydrogen.

In some embodiments of the compounds of Formulas IA-1 and IA-1a, R³ is hydrogen and R⁴ is OH or C₁-C₃alkoxy.

In some embodiments of the compounds of Formulas IA-1 and IA-1a, R³ is hydrogen, OH, or C₁-C₃alkoxy; R⁴ is H, OH, or C₁-C₃alkoxy, and wherein at least one of R³ and R⁴ is hydrogen.

In some embodiments of the compounds of Formulas IA-1 and IA-1a, R³ is hydrogen, OH, or C₁-C₃alkoxy; and R⁴ is hydrogen.

In some embodiments of the compounds of Formulas IA-1 and IA-1a, R³ is hydrogen; and R⁴ is hydrogen, OH, or C₁-C₃alkoxy.

In some embodiments of the compounds of Formulas IA-1 and IA-1a, R³ is hydrogen, OH, or OCH₃; R⁴ is hydrogen, OH, or OCH₃, and wherein at least one of R³ and R⁴ is hydrogen.

In some embodiments of the compounds of Formulas IA-1 and IA-1a, R³ is hydrogen, OH, or OCH₃; and R⁴ is hydrogen.

In some embodiments of the compounds of Formulas IA-1 and IA-1a, R³ is hydrogen; and R⁴ is hydrogen, OH, or OCH₃.

In some embodiments of the compounds of Formulas IA-1 and IA-1a, R³ is hydrogen or OH; R⁴ is hydrogen or OH, and wherein at least one of R³ and R⁴ is hydrogen.

In some embodiments, the compounds of Formula IA are compounds of Formula IA-2:

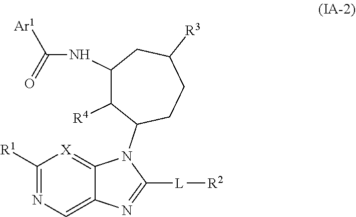

or a pharmaceutically acceptable salt thereof, wherein R³ is hydrogen, OH, C₁-C₃alkoxy, cyano, OSO₂(C₁-C₃alkyl), OSO₂(C₁-C₃alkyl), N₃, NH₂, NH(C₁-C₆alkyl), or N(C₁-C₆alkyl)₂; R⁴ is hydrogen, OH, C₁-C₃alkoxy, cyano, OSO₂(C₁-C₃alkyl), OSO₂(C₁-C₃alkyl), N₃, NH₂, NH(C₁-C₆alkyl), or N(C₁-C₆alkyl)₂, and wherein at least one of R³ and R⁴ is hydrogen; and Ar¹, R¹, R², L, and X are defined herein.

In some embodiments of the compounds of IA-2, R³ and R⁴ are each independently hydrogen, deuterium, —OH, halo, cyano, C₁-C₃alkyl, C₁-C₃haloalkyl, C₁-C₃alkoxy, C₁-C₃haloalkoxy, —NH₂, —NH(C₁-C₆alkyl), —N(C₁-C₆alkyl)₂, or deuterated C₁-C₃alkoxy.

In some embodiments of the compounds of IA-2, R³ and R⁴ are each independently hydrogen, —OH, halo, cyano, C₁-C₃alkyl, C₁-C₃haloalkyl, C₁-C₃alkoxy, C₁-C₃haloalkoxy, —NH₂, —NH(C₁-C₆alkyl), or —N(C₁-C₆alkyl)₂.

In some embodiments, the compounds of Formula IA-2 are compounds of Formula IA-2a:

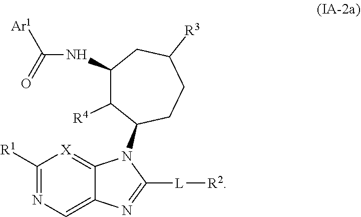

In some embodiments of the compounds of Formulas IA-2 and IA-2a, at least one of R³ and R⁴ is hydrogen.

In some embodiments of the compounds of Formulas IA-2 and IA-2a, at least one of R³ and R⁴ is deuterium.

In some embodiments of the compounds of Formulas IA-2 and IA-2a, R³ is hydrogen, OH, or C₁-C₃alkoxy; R⁴ is hydrogen, OH, or C₁-C₃alkoxy, and wherein at least one of R³ and R⁴ is hydrogen.

In some embodiments of the compounds of Formulas IA-2 and IA-2a, R³ is hydrogen, OH, or OCH₃; R⁴ is hydrogen, OH, or OCH₃, and wherein at least one of R³ and R⁴ is hydrogen.

In some embodiments of the compounds of Formulas IA-2 and IA-2a, R³ is hydrogen or OH; R⁴ is hydrogen or OH, and wherein at least one of R³ and R⁴ is hydrogen.

In some embodiments of the compounds of Formulas IA-2 and IA-2a, R³ is hydrogen and R⁴ is OH or C₁-C₃alkoxy.

In some embodiments, the compounds of Formula IA are compounds of Formula IA-3:

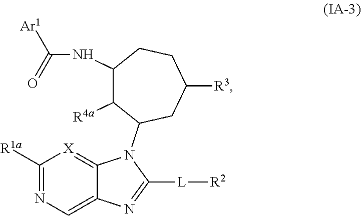

or a pharmaceutically acceptable salt thereof, wherein R³ is hydrogen, OH, C₁-C₃alkoxy, cyano, OCO₂(C₁-C₃alkyl), OSO₂(C₁-C₃alkyl), N₃, NH₂, NH(C₁-C₆alkyl), or N(C₁-C₆alkyl)₂; R⁴ is hydrogen, OH, C₁-C₃alkoxy, cyano, OCO₂(C₁-C₃alkyl), OSO₂(C₁-C₃alkyl), N₃, NH₂, NH(C₁-C₆alkyl), or N(C₁-C₆alkyl)₂, and wherein at least one of R³ and R⁴ is hydrogen, and Ar¹, R¹, R², L, and X are defined herein.

In some embodiments of the compounds of IA-3, R³ and R⁴ are each independently hydrogen, deuterium, —OH, halo, cyano, C₁-C₃alkyl, C₁-C₃haloalkyl, C₁-C₃alkoxy, C₁-C₃haloalkoxy, —NH₂, —NH(C₁-C₆alkyl), —N(C₁-C₆alkyl)₂, or deuterated C₁-C₃alkoxy.

In some embodiments of the compounds of IA-3, R³ and R⁴ are each independently hydrogen, —OH, halo, cyano, C₁-C₃alkyl, C₁-C₃haloalkyl, C₁-C₃alkoxy, C₁-C₃haloalkoxy, —NH₂, —NH(C₁-C₆alkyl), or —N(C₁-C₆alkyl)₂.

In some embodiments, the compounds of Formula IA-3 are compounds of Formula IA-3a:

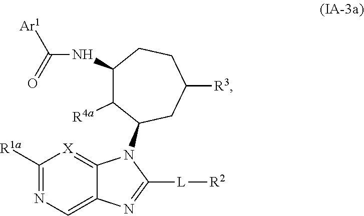

or a pharmaceutically acceptable salt thereof.

In some embodiments of the compounds of Formulas IA-3 and IA-3a, at least one of R³ and R⁴ is hydrogen.

In some embodiments of the compounds of Formulas IA-3 and IA-3a, at least one of R³ and R⁴ is deuterium.

In some embodiments of the compounds of Formulas IA-3 and IA-3a, R³ is hydrogen, OH, or C₁-C₃alkoxy; R⁴ is hydrogen, OH, or C₁-C₃alkoxy, and wherein at least one of R³ and R⁴ is hydrogen.

In some embodiments of the compounds of Formulas IA-3 and IA-3a, R³ is hydrogen, OH, or OCH₃; R⁴ is hydrogen, OH, or OCH₃, and wherein at least one of R³ and R⁴ is hydrogen.

In some embodiments of the compounds of Formulas IA-3 and IA-3a, R³ is hydrogen or OH; R⁴ is hydrogen or OH, and wherein at least one of R³ and R⁴ is hydrogen.

In some embodiments of the compounds of Formulas IA-3 and IA-3a, R³ is hydrogen and R⁴ is OH or C₁-C₃alkoxy.

In some embodiments, the compounds of Formula IA are compounds of Formula IA-4:

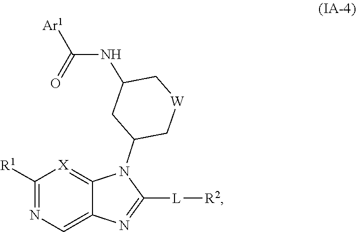

or a pharmaceutically acceptable salt thereof, wherein W is N—R⁵, S, S═O, SO₂, or O; and R⁵ is hydrogen, C₁-C₆alkyl, C(O)C₁-C₆alkyl, oxo, OH, C₁-C₆alkoxy, C(O)NR^{b}R^{c}(wherein each R^{b} and R^{c} is independently hydrogen or C₁-C₄alkyl), C₁-C₆hydroxyalkyl, CO₂(C₁-C₆alkyl), SO₂(C₁-C₆alkyl), or SO₂(C₃-C₆cycloalkyl), and Ar¹, R¹, R², L, and X are defined herein.

In some embodiments, the compounds of Formula IA-4 are compounds of Formula IA-4a:

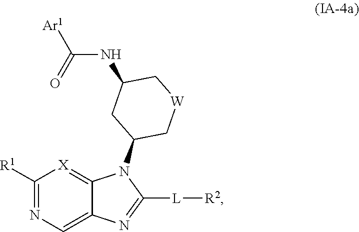

or a pharmaceutically acceptable salt thereof.

In some embodiments of the compounds of Formulas IA-4 and IA-4a, W is N—R⁵, S, S═O, SO₂, or O; and R⁵ is hydrogen, C₁-C₆alkyl, C(O)C₁-C₆alkyl, oxo, OH, or C₁-C₆alkoxy.

In some embodiments of the compounds of Formulas IA-4 and IA-4a, W is N—R⁵ wherein R⁵ is hydrogen, C₁-C₆alkyl, C(O)C₁-C₆alkyl, oxo, OH, C₁-C₆alkoxy, C(O)NR^{b}R^{c}(wherein each R^{b} and R^{c} is independently hydrogen or C₁-C₄alkyl), C₁-C₆hydroxyalkyl, CO₂(C₁-C₆alkyl), SO₂(C₁-C₆alkyl), or SO₂(C₃-C₆cycloalkyl). In other embodiments, R⁵ is hydrogen, C₁-C₆alkyl, C(O)C₁-C₆alkyl, oxo, OH, or C₁-C₆alkoxy.

In some embodiments of the compounds of Formulas IA-4 and IA-4a, W is N—R⁵, and R⁵ is CH₃, COCH₃, CO₂CH₃, or SO₂CH₃.

In some embodiments of the compounds of Formulas IA-4 and IA-4a wherein R⁵ is hydrogen, W is NH.

In some embodiments of the compounds of Formulas IA-4 and IA-4a wherein R⁵ is C₁-C₆alkyl, W is N(C₁-C₆alkyl).

In some embodiments of the compounds of Formulas IA-4 and IA-4a wherein R⁵ is C(O)C₁-C₆alkyl, W is NC(O)C₁-C₆alkyl.

In some embodiments of the compounds of Formulas IA-4 and IA-4a wherein R⁵ is ═O, W is N═O, i.e.,

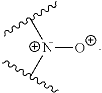 [SMILES: *[NH+](*)[OH2+]]

In some embodiments of the compounds of Formulas IA-4 and IA-4a wherein R⁵ is OH, W is N—OH.

In some embodiments of the compounds of Formulas IA-4 and IA-4a wherein R⁵ is C₁-C₆alkoxy, W is N—C₁-C₆alkoxy.

In some embodiments of the compounds of Formulas IA-4 and IA-4a wherein R⁵ is C(O)NR^{b}N^{c} and R^{b} and R^{c} are each hydrogen, W is N—C(O)NH₂.

In some embodiments of the compounds of Formulas IA-4 and IA-4a wherein R⁵ is C₁-C₆hydroxyalkyl, W is N—C₁-C₆hydroxyalkyl.

In some embodiments of the compounds of Formulas IA-4 and IA-4a wherein R⁵ is CO₂(C₁-C₆alkyl), W is N—CO₂(C₁-C₆alkyl).

In some embodiments of the compounds of Formulas IA-4 and IA-4a wherein R⁵ is SO₂(C₁-C₆alkyl), W is N—SO₂(C₁-C₆alkyl).

In some embodiments of the compounds of Formulas IA-4 and IA-4a wherein R⁵ is SO₂(C₃-C₆cycloalkyl), W is N—SO₂(C₃-C₆cycloalkyl).

In some embodiments of the compounds of Formulas IA-4 and IA-4a, W is O.

In some embodiments of the compounds of Formulas IA-4 and IA-4a, W is S.

In some embodiments of the compounds of Formulas IA-4 and IA-4a, W is S═O.

In some embodiments of the compounds of Formulas IA-4 and IA-4a, W is SO₂.

In some embodiments, the compounds of Formula IA are compounds of Formula IA-5:

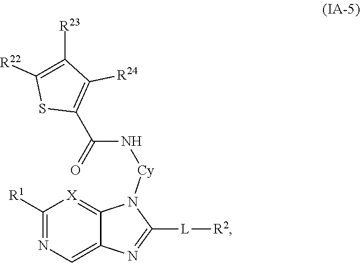

or a pharmaceutically acceptable salt thereof,
wherein R²² is halo, C₁-C₆alkyl, C₁-C₆haloalkyl, optionally substituted C₂-C₆alkenyl, cyano, C₃-C₆cycloalkyl, or optionally substituted C₂-C₆alkynyl;
each R²³ and R²⁴ is independently hydrogen, deuterium, halo, C₁-C₆alkyl, C₁-C₆haloalkyl, optionally substituted C₂-C₆alkenyl, cyano, C₃-C₆cycloalkyl, or optionally substituted C₂-C₆alkynyl; and Cy, L, R¹, R², and X are defined herein.

In some embodiments of the compounds of Formula IA-5, at least one of R²³ and R²⁴ is hydrogen. In other embodiments, both R²³ and R²⁴ are hydrogen.

In some embodiments of the compounds of Formula IA-5, the C₂-C₆alkenyl and C₂-C₆alkynyl groups are unsubstituted. In other embodiments, the C₂-C₆alkenyl and C₂-C₆alkynyl groups are independently substituted with C₁-C₆haloalkyl, cyano, or C₃-C₆cycloalkyl.

In some embodiments of the compounds of Formula IA-5, R²² is halo or ethynyl.

In some embodiments of the compounds of Formula IA-5, R²² is halo (—F, —Cl, —Br, or —I).

In some embodiments of the compounds of Formula IA-5, R²² is —Cl.

In some embodiments of the compounds of Formula IA-5, R²² is —Br.

In some embodiments of the compounds of Formula IA-5, R²² is C₁-C₆alkyl, such as, for example, C₆alkyl, C₅alkyl, C₄alkyl such as —CH₂CH(CH₃)₂, C₃alkyl such as —CH(CH₃)₂, C₂alkyl such as —CH₂CH₃, or C₁alkyl such as —CH₃.

In some embodiments of the compounds of Formula IA-5, R²² is C₁-C₆haloalkyl, such as, for example, C₆haloalkyl, C₅haloalkyl, C₄haloalkyl, C₃haloalkyl such as —CH(CH₃)(CF₃), C₂haloalkyl such as —CH₂CF₃, or C₁haloalkyl such as —CF₃, —CHF₂, or —CH₂F.

In some embodiments of the compounds of Formula IA-5, R²² is optionally substituted C₂-C₆alkenyl, such as, for example, optionally substituted: C₆ alkenyl, C₅ alkenyl, C₄ alkenyl, C₃ alkenyl such as —CH═CHCH₃, or C₂ alkenyl such as —CH═CH₂.

In some embodiments of the compounds of Formula IA-5, R²² is cyano.

In some embodiments of the compounds of Formula IA-5, R²² is C₃-C₆cycloalkyl, such as, for example, C₆cycloalkyl (e.g., cyclohexyl), C₅cycloalkyl (e.g., cyclopentyl), C₄cycloalkyl (e.g., cyclobutyl), or C₃cycloalkyl (e.g., cyclopropyl).

In some embodiments of the compounds of Formula IA-5, R²² is optionally substituted C₂-C₆alkynyl, such as, for example, optionally substituted: C₆ alkynyl, C₅ alkynyl, C₄ alkynyl, C₃ alkynyl, or C₂ alkynyl.

In some embodiments of the compounds of Formula IA-5, R²² is ethynyl (i.e., —C≡CH).

In some embodiments of the compounds of Formula IA-5, R²² is halo or optionally substituted C₂-C₆alkynyl; and R²³ and R²⁴ are hydrogen.

In some embodiments, the compounds of Formula IA are compounds of Formula IA-6:

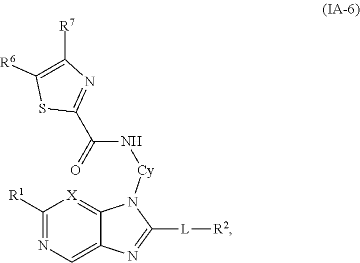

or a pharmaceutically acceptable salt thereof, wherein R⁶ is halo, C₁-C₆alkyl, C₁-C₆haloalkyl, optionally substituted C₂-C₆alkenyl, cyano, C₃-C₆cycloalkyl, or optionally substituted C₂-C₆alkynyl; R⁷ is hydrogen, deuterium, halo, C₁-C₆alkyl, C₁-C₆haloalkyl, optionally substituted C₂-C₆alkenyl, cyano, C₃-C₆cycloalkyl, or optionally substituted C₂-C₆alkynyl; and Cy, L, R¹, R², and X are defined herein.

In some embodiments of the compounds of Formula IA-6, R⁷ is hydrogen.

In some embodiments of the compounds of Formula IA-6, the C₂-C₆alkenyl and C₂-C₆alkynyl groups are unsubstituted. In other embodiments, the C₂-C₆alkenyl and C₂-C₆alkynyl groups are independently substituted with C₁-C₆haloalkyl, cyano, or C₃-C₆cycloalkyl.

In some embodiments of the compounds of Formula IA-6, R⁶ is halo, C₁-C₆haloalkyl, cyano, or ethynyl.

In some embodiments of the compounds of Formula IA-6, R⁶ is halo (—F, —Cl, —Br, or —I).

In some embodiments of the compounds of Formula IA-6, R⁶ is —Cl.

In some embodiments of the compounds of Formula IA-6, R⁶ is —Br.

In some embodiments of the compounds of Formula IA-6, R⁶ is C₁-C₆alkyl, such as, for example, C₆alkyl, C₅alkyl, C₄alkyl such as —CH₂CH(CH₃)₂, C₃alkyl such as —CH(CH₃)₂, C₂alkyl such as —CH₂CH₃, or C₁alkyl such as —CH₃.

In some embodiments of the compounds of Formula IA-6, R⁶ is C₁-C₆haloalkyl, such as, for example, C₆haloalkyl, C₅haloalkyl, C₄haloalkyl, C₃haloalkyl such as —CH(CH₃)(CF₃), C₂haloalkyl such as —CH₂CF₃, or C₁haloalkyl such as —CF₃, —CHF₂, or —CH₂F.

In some embodiments of the compounds of Formula IA-6, R⁶ is optionally substituted C₂-C₆alkenyl, such as, for example, optionally substituted: C₆ alkenyl, C₅ alkenyl, C₄ alkenyl, C₃ alkenyl such as —CH═CHCH₃, or C₂ alkenyl such as —CH═CH₂.

In some embodiments of the compounds of Formula IA-6, R⁶ is cyano.

In some embodiments of the compounds of Formula IA-6, R⁶ is C₃-C₆cycloalkyl, such as, for example, C₆cycloalkyl (e.g., cyclohexyl), C₅cycloalkyl (e.g., cyclopentyl), C₄cycloalkyl (e.g., cyclobutyl), or C₃cycloalkyl (e.g., cyclopropyl).

In some embodiments of the compounds of Formula IA-6, R⁶ is optionally substituted C₂-C₆alkynyl, such as, for example, optionally substituted: C₆ alkynyl, C₅ alkynyl, C₄ alkynyl, C₃ alkynyl, or C₂ alkynyl.

In some embodiments of the compounds of Formula IA-6, R⁶ is ethynyl (i.e., —C≡CH).

In some embodiments of the compounds of Formula IA-6, R⁶ is halo, C₁-C₆haloalkyl, cyano, or optionally substituted C₂-C₆alkynyl; and R⁷ is hydrogen.

In some embodiments, the compounds of Formula IA are compounds of Formula IA-7:

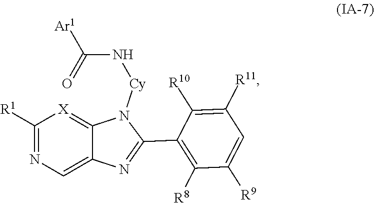

or a pharmaceutically acceptable salt thereof, wherein R⁸, R⁹, R¹⁰, and R¹¹ are each independently hydrogen, halo, C₁-C₄alkyl, C₃-C₄cycloalkyl, C₁-C₄alkoxy, C₁-C₄haloalkoxy, cyano, OH, NR^{b}R^{c}, C(O)NR^{b}R^{c}, CO₂(C₁-C₄alkyl), COOH, C₁-C₄alkylene-R²⁹, or O—C₁-C₄alkylene-R³⁰; R²⁹ is cyano, NR^{b}R^{c}, or COOH; R³⁰ is OH, OCH₃, or NR^{b}R^{c};
   each R^{b} and each R^{c} is independently hydrogen or C₁-C₄alkyl; and
   Ar¹, Cy, R¹, and X are defined herein.

In some embodiments of the compounds of Formula IA-7, R⁸, R⁹, R¹⁰, and R¹¹ are each independently hydrogen, halo, C₁-C₄alkyl, C₃-C₄cycloalkyl, C₁-C₄alkoxy, cyano, NR^{b}R^{c}, C(O)NR^{b}R^{c}, C(O)OC₁-C₄alkyl, COOH, or C₁₋₄alkyleneCO₂H, wherein each R^{b} and each R^{c} is independently hydrogen or C₁-C₄alkyl.

In some embodiments of the compounds of Formula IA-7, at least two of R⁸, R⁹, R¹⁰, and R¹¹ are hydrogen.

In some embodiments of the compounds of Formula IA-7, at least three of R⁸, R⁹, R¹⁰, and R¹¹ are hydrogen.

In some embodiments of the compounds of Formula IA-7, at least one of R⁸, R⁹, R¹⁰, and R¹¹ is F, CH₃, OCH₃, OH, or cyano.

In some embodiments of the compounds of Formula IA-7, at least one of R⁸, R⁹, R¹⁰, and R¹¹ is —F, —CH₃, —OCH₃, —NH₂, or cyano.

In some embodiments of the compounds of Formula IA-7, R⁸ is —F and R⁹, R¹⁰, and R¹¹ are each hydrogen.

In some embodiments of the compounds of Formula IA-7, R⁸ is —COOH or C₁₋₄alkylenecarboxylic acid and R⁹, R¹⁰, and R¹¹ are each hydrogen.

In some embodiments, the compounds of Formula IA are compounds of Formula IA-8:

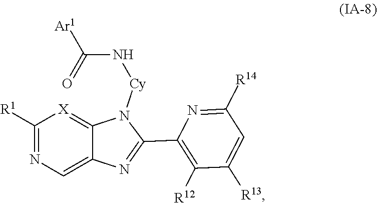

or a pharmaceutically acceptable salt thereof, wherein R⁸, R⁹, R¹⁰, and R¹¹ are each independently hydrogen, halo, C₁-C₄alkyl, C₃-C₄cycloalkyl, C₁-C₄alkoxy, C₁-C₄haloalkoxy, cyano, OH, NR^{b}R^{c}, C(O)NR^{b}R^{c}, CO₂(C₁-C₄alkyl), COOH, C₁-C₄alkylene-R²⁹, or O—C₁-C₄alkylene-R³⁰; R²⁹ is cyano, NR^{b}R^{c}, or COOH; R³⁰ is OH, OCH₃, or NR^{b}R^{c};
   each R^{b} and each R^{c} is independently hydrogen or C₁-C₄alkyl; and
   Ar¹, Cy, R¹, and X are defined herein.

In some embodiments of the compounds of Formula IA-8, R⁸, R⁹, R¹⁰, and R¹¹ are each independently hydrogen, halo, C₁-C₄alkyl, C₃-C₄cycloalkyl, C₁-C₄alkoxy, cyano, NR^{b}R^{c}, C(O)NR^{b}R^{c}, C(O)OC₁-C₄alkyl, COOH, or C₁₋₄alkyleneCO₂H, wherein each R^{b} and each R^{c} is independently hydrogen or C₁-C₄alkyl.

In some embodiments of the compounds of Formula IA-8, at least two of R¹², R¹³, and R¹⁴ are hydrogen.

In some embodiments of the compounds of Formula IA-8, at least one of R¹², R¹³, and R¹⁴ is —F, —CH₃, —OCH₃, —NH₂, or cyano.

In some embodiments of the compounds of Formula IA-8, at least one of R¹², R¹³, and R¹⁴ is F or CH₃.

In some embodiments of the compounds of Formula IA-8, R¹² is —F and R¹³ and R¹⁴ are each hydrogen.

In some embodiments of the compounds of Formula IA-8, R¹² is —F or —CH₃ and R¹³ and R¹⁴ are each hydrogen.

In some embodiments, the compounds of Formula IA are compounds of Formula IA-9:

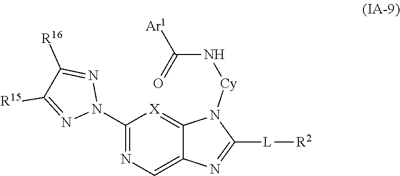

or a pharmaceutically acceptable salt thereof, wherein R¹⁵ and R¹⁶ are each independently hydrogen, halo, C₁-C₆alkyl, C₁-C₆haloalkyl, C₁-C₆hydroxyalkyl, CO₂(C₁-C₆alkyl), or CONR^{b}R^{c};
   each R^{b} and each R^{c} is independently hydrogen or C₁-C₄alkyl; and
   Ar¹, Cy, L, R², and X are defined herein.

In some embodiments of the compounds of Formula IA-9, R¹⁵ and R¹⁶ are each independently hydrogen, halo, C₁-C₆alkyl, C₁-C₆hydroxyalkyl, C(O)OC₁-C₆alkyl, or CONR^{b}R^{c}.

In some embodiments of the compounds of Formula IA-9, R¹⁵ and R¹⁶ are each independently hydrogen, halo, C₁-C₆alkyl, C(O)OC₁-C₆alkyl, or CONR^{b}R^{c}.

In some embodiments of the compounds of Formula IA-9, at least one of R¹⁵ and R¹⁶ is hydrogen.

In some embodiments of the compounds of Formula IA-9, one of R¹⁵ or R¹⁶ is hydrogen and the other of R¹⁵ or R¹⁶ is hydrogen, CH₃, CO₂CH₃, CH₂OH, CH(OH)(CH₃), or C(CH₃)₂OH.

In some embodiments of the compounds of Formula IA-9, one of R¹⁵ and R¹⁶ is halo or —CH₃.

In some embodiments of the compounds of Formula IA-9, one of R¹⁵ and R¹⁶ is —CH₃, —COOCH₃, or —C(CH₃)₂OH.

In some embodiments, the compounds of Formula IA are compounds of Formula IA-10:

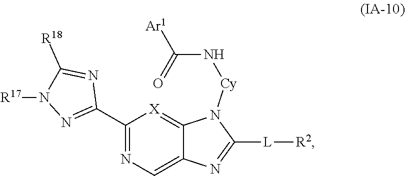

or a pharmaceutically acceptable salt thereof, wherein R¹⁷ is hydrogen, C₁-C₆alkyl, or C₁-C₆haloalkyl;
R¹⁸ is hydrogen, halo, C₁-C₆alkyl, CO₂(C₁-C₆alkyl), or CONR^{b}R^{c}; each R^{b} and each R^{c} is independently hydrogen or C₁-C₄alkyl; and
Ar¹, Cy, L, R², and X are defined herein.

In some embodiments of the compounds of Formula IA-10, R¹⁷ is hydrogen. When R¹⁷ is hydrogen, compounds of Formula IA-10 can also exist in the tautomeric forms shown in Formula IA-10(T1) and Formula IA-10(T2):

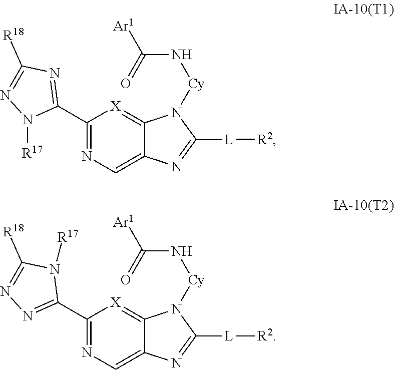

In some embodiments of the compounds of Formula IA-10, both R¹⁷ and R¹⁸ are hydrogen.

In some embodiments of the compounds of Formula IA-10, R¹⁷ is hydrogen, CH₃, or CHF₂.

In some embodiments of the compounds of Formula IA-10, R¹⁸ is hydrogen or C₁-C₄alkyl.

In some embodiments, the compounds of Formula IA are compounds of Formula IA-11:

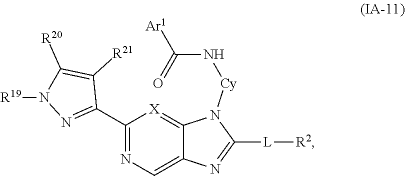

or a pharmaceutically acceptable salt thereof, wherein R¹⁹ is hydrogen, C₁-C₆alkyl, or C₁-C₆haloalkyl;
R²⁰ and R²¹ are each independently hydrogen, halo, C₁-C₆alkyl, C₁-C₆haloalkyl, CO₂(C₁-C₆alkyl), or CONR^{b}R^{c};
   each R^{b} and each R^{c} is independently hydrogen or C₁-C₄alkyl; and
   Ar¹, Cy, L, R², and X are defined herein.

In some embodiments of the compounds of Formula IA-11, one of R²⁰ and R²¹ is C₁-C₄alkyl or C₁-C₄haloalkyl.

In some embodiments of the compounds of Formula IA-11, R¹⁹ is hydrogen. When R¹⁹ is hydrogen, compounds of Formula IA-11 can also exist in the tautomeric form shown in Formula IA-11(T):

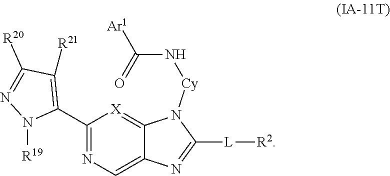

In some embodiments, the compounds of Formula IA are compounds of Formula IA-12:

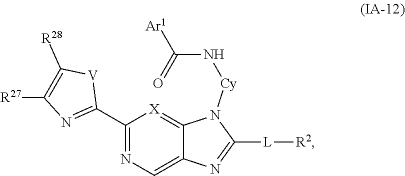

or a pharmaceutically acceptable salt thereof, wherein:
V is O or S;
R²⁷ is hydrogen, C₁-C₆alkyl, C₁-C₆haloalkyl, halo, C₁-C₆hydroxyalkyl, or NR^{b}R^{c};
R²⁸ is hydrogen or C₁-C₆alkyl; and
each R^{b} and R^{c} is independently hydrogen or C₁-C₄alkyl and Ar¹, Cy, L, R², and X are defined herein.

In some embodiments of the compounds of Formula IA-12, R²⁷ is hydrogen or C₁-C₄alkyl and R²⁸ is hydrogen.

In some embodiments of the compounds of Formula IA-12, R²⁷ is hydrogen, CH₃, CH₂OH, NH₂, or Cl, and R²⁸ is hydrogen.

In some embodiments, the compounds of Formula IA are compounds of Formula IA-13:

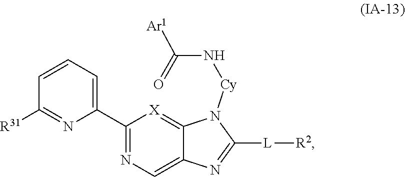

or a pharmaceutically acceptable salt thereof, wherein:
R³¹ is hydrogen, C₁-C₆alkyl, C₁-C₆haloalkyl, halo, C₁-C₆alkoxy, C₁-C₆hydroxyalkyl, cyano, or NR^{b}R^{c} (wherein each R^{b} and R^{c} is independently hydrogen or C₁-C₄alkyl), and Ar¹, Cy, L, R², and X are defined herein.

In some embodiments of the compounds of Formula IA-13, R³¹ is NH₂, F, OCH₃, CH₂OH, or cyano.

In some embodiments, the compounds of Formula IB are compounds of Formula IB-1:

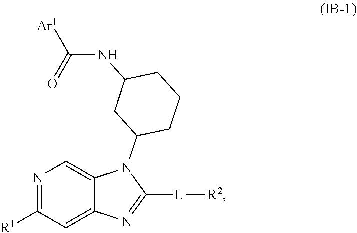

or a pharmaceutically acceptable salt thereof, wherein Ar¹, L, R¹, and R² are defined herein.

In some embodiments, the compounds of Formula IB-1 are compounds of Formula IB-1a:

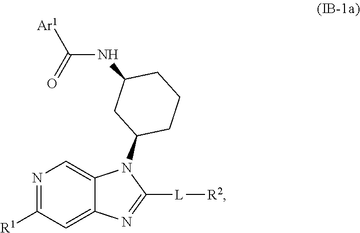

or a pharmaceutically acceptable salt thereof.

In some embodiments, the compounds of Formula IB-1 or IB-1a are those wherein R¹ is optionally substituted triazolyl, optionally substituted oxadiazolyl, optionally substituted pyrazolyl, or optionally substituted oxazolyl.

In some embodiments, the compounds of Formula IB-1 or IB-1a are those wherein L is a bond and R² is optionally substituted phenyl or optionally substituted pyridinyl.

In some embodiments, the compounds of Formula IB-1 or IB-1a are those wherein Ar¹ is optionally substituted thiophenyl or optionally substituted thiazolyl.

In some embodiments, the compounds of formula IIIA are compounds of Formula IIIA-1:

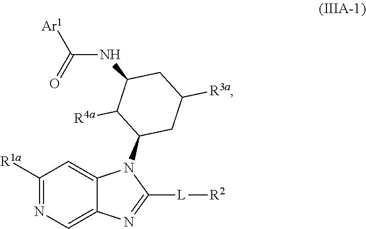

or a pharmaceutically acceptable salt thereof.

In some embodiments, the compounds of formula IIIB are compounds of Formula IIIB-1:

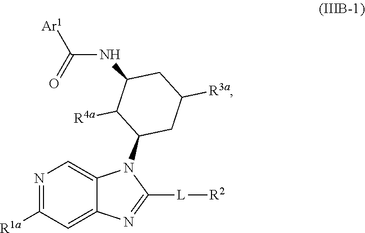

or a pharmaceutically acceptable salt thereof.

In some embodiments, the compounds of formula IIIC are compounds of Formula IIIC-1:

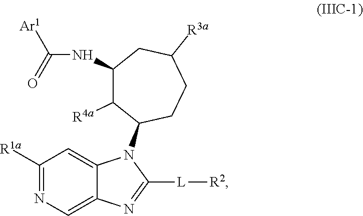

or a pharmaceutically acceptable salt thereof.

In some embodiments, the compounds of formula IIID are compounds of Formula IIID-1:

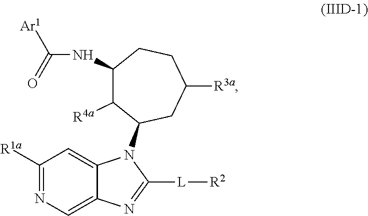

or a pharmaceutically acceptable salt thereof.

In some aspects, R^{1a} in the compounds of Formula IIIA, Formula IIIA-1, Formula IIIB, Formula IIIB-1, Formula IIIC, Formula IIIC-1, Formula IIID, or Formula IIID-1 is hydrogen, C₁-C₆alkyl, C₁-C₆alkoxy, halo, C₁-C₆haloalkyl, C₁-C₆haloalkoxy, CONR^{j}(C₁-C₄alkyl), CONR^{j}(C₃-C₄cycloalkyl), or NR^{b}R^{c}, wherein each R^{j}, R^{b}, and R^{c} is independently hydrogen or C₁-C₄alkyl.

In some embodiments, R^{1a} is hydrogen, C₁-C₄alkoxy, halo, CONH(C₁-C₄alkyl), or NR^{b}R^{c}, wherein R^{b} and R^{c} are each independently hydrogen or C₁-C₄alkyl.

In other embodiments, R^{1a} is hydrogen, NH₂, NHCH₃, F, OCH₃, or C(O)NH(CH₃).

In some aspects, one of R^{3a} or R^{4a} in the compounds of Formula IIIA, Formula IIIA-1, Formula IIIB, Formula IIIB-1, Formula IIIC, Formula IIIC-1, Formula IIID, or Formula IIID-1 is hydrogen, deuterium, OH, cyano, C₁-C₃alkyl, C₁-C₃haloalkyl, C₁-C₃alkoxy, C₁-C₃haloalkoxy, deuterated C₁-C₃alkoxy, NH₂, NH(C₁-C₄alkyl), N(C₁-C₄alkyl)₂, OCO₂(C₁-C₄alkyl), OCO₂(C₃-C₄cycloalkyl), OSO₂(C₁-C₄alkyl), OSO₂(C₃-C₄cycloalkyl), OC(O)(C₁-C₄alkyl), OC(O)(C₃-C₄cycloalkyl), or NR^{m}Rⁿ; and the other of R^{3a} or R^{4a} is OH, cyano, C₁-C₃alkyl, C₁-C₃haloalkyl, C₁-C₃alkoxy, C₁-C₃haloalkoxy, deuterated C₁-C₃alkoxy, NH₂, NH(C₁-C₄alkyl), N(C₁-C₄alkyl)₂, OCO₂(C₁-C₄alkyl), OCO₂(C₃-C₄cycloalkyl), OSO₂(C₁-C₄alkyl), OSO₂(C₃-C₄cycloalkyl), OC(O)(C₁-C₄alkyl), OC(O)(C₃-C₄cycloalkyl), or NR^{m}Rⁿ; and wherein each R^{m} is independently hydrogen or C₁-C₄alkyl and each Rⁿ is independently CO₂(C₁-C₄alkyl), C(O)C₁-C₄alkyl, SO₂C₁-C₄alkyl, or SO₂(C₃-C₄cycloalkyl).

In some embodiments, one of R^{3a} or R^{4a} is hydrogen, deuterium, OH, cyano, C₁-C₃alkyl, C₁-C₃haloalkyl, C₁-C₃alkoxy, C₁-C₃haloalkoxy, deuterated C₁-C₃alkoxy, NH₂, NH(C₁-C₄alkyl), or N(C₁-C₄alkyl)₂; and the other of R^{3a} or R^{4a} is OH, cyano, C₁-C₃alkyl, C₁-C₃haloalkyl, C₁-C₃alkoxy, C₁-C₃haloalkoxy, deuterated C₁-C₃alkoxy, NH₂, NH(C₁-C₄alkyl), or N(C₁-C₄alkyl)₂.

In some embodiments, one of R^{3a} or R^{4a} is OH or C₁-C₃alkoxy; and the other of R^{3a} or R^{4a} is hydrogen.

In some embodiments, the compounds of formula IVA are compounds of Formula IVA-1:

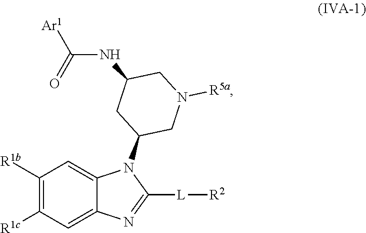

or a pharmaceutically acceptable salt thereof.

In some embodiments, the compounds of formula IVB are compounds of Formula IVB-1:

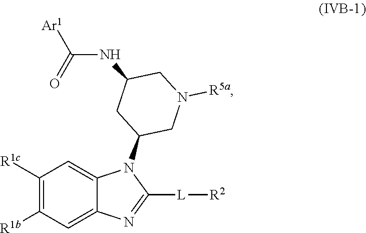

or a pharmaceutically acceptable salt thereof.

In some aspects, R^{1b} in the compounds of Formula IVA, Formula IVA-1, Formula IVB, or Formula IVB-1 is optionally substituted heteroaryl, optionally substituted aryl, optionally substituted 4-, 5- or 6-membered heterocycloalkyl, CONR^{j}(C₁-C₄alkyl), CONR^{j}(C₃-C₄cycloalkyl), or cyano, wherein each R^{j} is independently hydrogen or C₁-C₄alkyl.

In some embodiments, R^{1b} is optionally substituted 5- or 6-membered heteroaryl, such as, for example, optionally substituted: pyrrolyl, pyrazolyl, imidazolyl, triazolyl, tetrazolyl, oxadiazolyl, oxazolyl, isoxazolyl, isothiazolyl, thiazolyl, thiadiazolyl, pyridinyl, pyrimidinyl, pyrazinyl, or pyridazinyl.

In other embodiments, R^{1b} is unsubstituted 5- or 6-membered heteroaryl, such as, for example, unsubstituted: pyrrolyl, pyrazolyl, imidazolyl, triazolyl, tetrazolyl, oxadiazolyl, oxazolyl, isoxazolyl, isothiazolyl, thiazolyl, thiadiazolyl, pyridinyl, pyrimidinyl, pyrazinyl, or pyridazinyl.

In further embodiments, R^{1b} is unsubstituted oxadiazolyl. In yet other embodiments, R^{1b} is

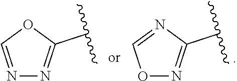 [SMILES: *c1nnco1 ; *c1ncon1]

In some embodiments, R^{1b} is CONR^{j}(C₁-C₄alkyl) or cyano. In other embodiments, R^{1b} is CONH(CH₃) or cyano. In yet other embodiments, R^{1b} is CONH(CH₃). In still other embodiments, R^{1b} is cyano.

In some aspects, R^{1c} in the compounds of Formula IVA, Formula IVA-1, Formula IVB, or Formula IVB-1 is hydrogen, halo, C₁-C₆alkyl, C₁-C₆haloalkyl, C₁-C₆alkoxy, or C₁-C₆haloalkoxy.

In some embodiments, R^{1c} is hydrogen or C₁-C₄alkoxy. In other embodiments, R^{1c} is hydrogen or OCH₃.

In some aspects, R^{5a} in the compounds of Formula IVA, Formula IVA-1, Formula IVB, or Formula IVB-1 is hydrogen, C₁-C₆alkyl, C(O)C₁-C₆alkyl, C(O)NR^{b}R^{c}, oxo, OH, C₁-C₆alkoxy, C₁-C₆hydroxyalkyl, CO₂(C₁-C₆alkyl), SO₂(C₁-C₆alkyl), or SO₂(C₃-C₆cycloalkyl), wherein each R^{b} and each R^{c} is independently hydrogen or C₁-C₄alkyl.

In some embodiments, R^{5a} is hydrogen, C₁-C₆alkyl, C(O)C₁-C₆alkyl, C(O)NR^{b}R^{c}, oxo, OH, or C₁-C₆alkoxy, wherein each R^{b} and each R^{c} is independently hydrogen or C₁-C₄alkyl.

In some embodiments, R^{5a} is C₁-C₄alkyl, C(O)C₁-C₄alkyl, or C(O)NR^{b}R^{c}, wherein each R^{b} and each R^{c} is independently hydrogen or C₁-C₄alkyl.

In some embodiments, R^{5a} is CH₃, C(O)CH₃, or C(O)NH₂.

In some embodiments, the compounds of formula VA are compounds of Formula VA-1:

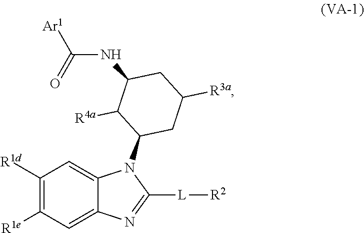

or a pharmaceutically acceptable salt thereof.

In some embodiments, the compounds of formula VB are compounds of Formula VB-1:

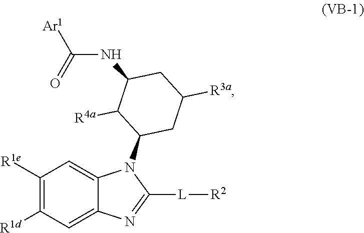

or a pharmaceutically acceptable salt thereof.

In some embodiments, the compounds of formula VC are compounds of Formula VC-1:

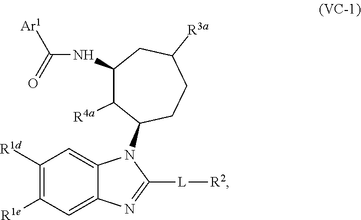

or a pharmaceutically acceptable salt thereof.

In some embodiments, the compounds of formula VD are compounds of Formula VD-1:

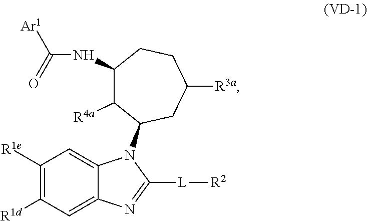

or a pharmaceutically acceptable salt thereof.

In some aspects, R^{1d} in the compounds of Formula VA, Formula VA-1, Formula VB, Formula VB-1, Formula VC, Formula VC-1, Formula VD, or Formula VD-1 is optionally substituted heteroaryl, optionally substituted aryl, optionally substituted 4-, 5- or 6-membered heterocycloalkyl, CONR^{j}(C₁-C₄alkyl), CONR^{j}(C₃-C₄cycloalkyl), or cyano, wherein each R^{j} is independently hydrogen or C₁-C₄alkyl.

In some embodiments, R^{1d} is optionally substituted 5- or 6-membered heteroaryl, such as, for example, optionally substituted: pyrrolyl, pyrazolyl, imidazolyl, triazolyl, tetrazolyl, oxadiazolyl, oxazolyl, isoxazolyl, isothiazolyl, thiazolyl, thiadiazolyl, pyridinyl, pyrimidinyl, pyrazinyl, or pyridazinyl.

In other embodiments, R^{1d} is unsubstituted 5- or 6-membered heteroaryl, such as, for example, unsubstituted: pyrrolyl, pyrazolyl, imidazolyl, triazolyl, tetrazolyl, oxadiazolyl, oxazolyl, isoxazolyl, isothiazolyl, thiazolyl, thiadiazolyl, pyridinyl, pyrimidinyl, pyrazinyl, or pyridazinyl.

In further embodiments, R^{1d} is optionally substituted oxadiazolyl, optionally substituted thiadiazolyl, optionally substituted oxazolyl, optionally substituted pyrazolyl, optionally substituted imidazolyl, optionally substituted triazolyl, optionally substituted tetrazolyl, optionally substituted thiazolyl, or optionally substituted pyridinyl, optionally substituted pyrimidinyl, or optionally substituted pyridazinyl. In still other embodiments, R^{1d} is optionally substituted oxazolyl, optionally substituted triazolyl, optionally substituted pyrimidinyl, or optionally substituted thiazolyl.

In some embodiments, R^{1d} is CONR^{j}(C₁-C₄alkyl). In other embodiments, R^{1d} is CONH(CH₃).

In some aspects, R^{1e} in the compounds of Formula VA, Formula VA-1, Formula VB, Formula VB-1, Formula VC, Formula VC-1, Formula VD, or Formula VD-1 is hydrogen, halo, C₁-C₆alkyl, C₁-C₆haloalkyl, C₁-C₆alkoxy, or C₁-C₆haloalkoxy.

In some embodiments, R^{1e} is hydrogen or C₁-C₄alkoxy. In other embodiments, R^{1e} is hydrogen or OCH₃. In yet other embodiments, R^{1e} is hydrogen.

In some embodiments, R^{1d} is CONH(CH₃) and R^{1e} is hydrogen.

In some aspects, one of R^{3a} or R^{4a} in the compounds of Formula VA, Formula VA-1, Formula VB, Formula VB-1, Formula VC, Formula VC-1, Formula VD, or Formula VD-1 is hydrogen, deuterium, OH, cyano, C₁-C₃alkyl, C₁-C₃haloalkyl, C₁-C₃alkoxy, C₁-C₃haloalkoxy, deuterated C₁-C₃alkoxy, NH₂, NH(C₁-C₄alkyl), N(C₁-C₄alkyl)₂, OCO₂(C₁-C₄alkyl), OSO₂(C₃-C₄cycloalkyl), OSO₂(C₁-C₄alkyl), OSO₂(C₃-C₄cycloalkyl), OC(O)(C₁-C₄alkyl), OC(O)(C₃-C₄cycloalkyl), or NR^{m}Rⁿ; and the other of R^{3a} or R^{4a} is OH, cyano, C₁-C₃alkyl, C₁-C₃haloalkyl, C₁-C₃alkoxy, C₁-C₃haloalkoxy, deuterated C₁-C₃alkoxy, NH₂, NH(C₁-C₄alkyl), N(C₁-C₄alkyl)₂, OCO₂(C₁-C₄alkyl), OSO₂(C₃-C₄cycloalkyl), OSO₂(C₁-C₄alkyl), OSO₂(C₃-C₄cycloalkyl), OC(O)(C₁-C₄alkyl), OC(O)(C₃-C₄cycloalkyl), or NR^{m}Rⁿ; and wherein each R^{m} is independently hydrogen or C₁-C₄alkyl and each Rⁿ is independently CO₂(C₁-C₄alkyl), C(O)C₁-C₄alkyl, SO₂(C₁-C₄alkyl), or SO₂(C₃-C₄cycloalkyl).

In some embodiments, one of R^{3a} or R^{4a} is hydrogen, deuterium, OH, cyano, C₁-C₃alkyl, C₁-C₃haloalkyl, C₁-C₃alkoxy, C₁-C₃haloalkoxy, deuterated C₁-C₃alkoxy, NH₂, NH(C₁-C₄alkyl), or N(C₁-C₄alkyl)₂; and the other of R^{3a} or R^{4a} is OH, cyano, C₁-C₃alkyl, C₁-C₃haloalkyl, C₁-C₃alkoxy, C₁-C₃haloalkoxy, deuterated C₁-C₃alkoxy, NH₂, NH(C₁-C₄alkyl), or N(C₁-C₄alkyl)₂.

In some embodiments, one of R^{3a} or R^{4a} is OH or C₁-C₃alkoxy; and the other of R^{3a} or R^{4a} is hydrogen.

In some embodiments, the compounds of Formula IIA, Formula IIB, Formula IIIA, Formula IIIA-1, Formula IIIB, Formula IIIB-1, Formula IIIC, Formula IIIC-1, Formula IIID, Formula IIID-1, Formula IVA, Formula IVA-1, Formula IVB, Formula IVB-1, Formula VA, Formula VA-1, Formula VB, Formula VB-1, Formula VC, Formula VC-1, Formula VD, or Formula VD-1 are those wherein L is a bond and R² is optionally substituted phenyl or optionally substituted pyridinyl.

In some embodiments, the compounds of Formula IIA, Formula IIB, Formula IIIA, Formula IIIA-1, Formula IIIB, Formula IIIB-1, Formula IIIC, Formula IIIC-1, Formula IIID, Formula IIID-1, Formula IVA, Formula IVA-1, Formula IVB, Formula IVB-1, Formula VA, Formula VA-1, Formula VB, Formula VB-1, Formula VC, Formula VC-1, Formula VD, or Formula VD-1 are those wherein L is a bond and R² is phenyl, substituted with one or more of halo, C₁-C₄alkyl, C₃-C₄cycloalkyl, C₁-C₄alkoxy, C₁-C₄haloalkoxy, cyano, OH, NR^{b}R^{c}, C(O)NR^{b}R^{c}, CO₂C₁-C₄alkyl, COOH, C₁-C₄alkylene-R²⁹, or O—C₁-C₄alkylene-R³⁰; R²⁹ is cyano, NR^{b}R^{c}, or COOH; R³⁰ is OH, OCH₃, or NR^{b}R^{c}; and each R^{b} and each R^{c} is independently hydrogen or C₁-C₄alkyl.

In some embodiments, the compounds of Formula IIA, Formula IIB, Formula IIIA, Formula IIIA-1, Formula IIIB, Formula IIIB-1, Formula IIIC, Formula IIIC-1, Formula IIID, Formula IIID-1, Formula IVA, Formula IVA-1, Formula IVB, Formula IVB-1, Formula VA, Formula VA-1, Formula VB, Formula VB-1, Formula VC, Formula VC-1, Formula VD, or Formula VD-1 are those wherein L is a bond and R² is pyridinyl substituted with one or more of halo or C₁-C₄alkyl. In other embodiments, L is a bond and R² is pyridinyl substituted with one fluoro or CH₃.

In some embodiments, the compounds of Formula IIA, Formula IIB, Formula IIIA, Formula IIIA-1, Formula IIIB, Formula IIIB-1, Formula IIIC, Formula IIIC-1, Formula IIID, Formula IIID-1, Formula IVA, Formula IVA-1, Formula IVB, Formula IVB-1, Formula VA, Formula VA-1, Formula VB, Formula VB-1, Formula VC, Formula VC-1, Formula VD, or Formula VD-1 are those wherein Ar¹ is optionally substituted thiophenyl or optionally substituted thiazolyl.

In some embodiments, the disclosure provides specific examples of Formulas IA and IB, and their pharmaceutically acceptable salts, tautomers, and/or isotopologues as set forth in Table 1 below.

TABLE 1

| Ex. | | |
|---|---|---|
| No. | Structure | Chemical Name |
| 1 | 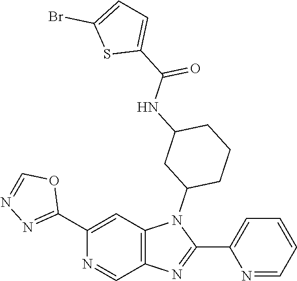 [SMILES: O=C(NC1CCCC(n2c(-c3ccccn3)nc3cnc(-c4nnco4)cc32)C1)c1ccc(Br)s1] | N-(3-(6-(1,3,4-oxadiazol-2-yl)-2- (pyridin-2-yl)-1H-imidazo[4,5- c]pyridin-1-yl)cyclohexyl)-5- bromothiophene-2-carboxamide(1) |
| 2 | 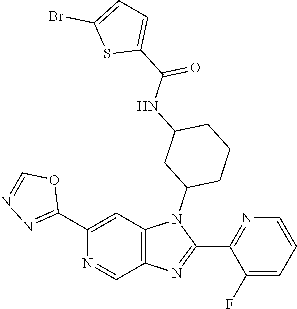 [SMILES: O=C(NC1CCCC(n2c(-c3ncccc3F)nc3cnc(-c4nnco4)cc32)C1)c1ccc(Br)s1] | 5-bromo-N-(3-(2-(3-fluoropyridin-2- yl)-6-(1,3,4-oxadiazol-2-yl)-1H- imidazo[4,5-c]pyridin-1- yl)cyclohexyl)thiophene-2- carboxamide(1) |
| 3 | 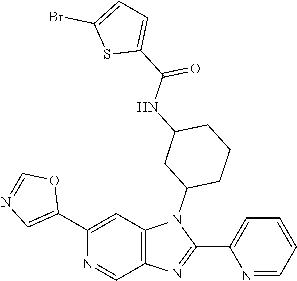 [SMILES: O=C(NC1CCCC(n2c(-c3ccccn3)nc3cnc(-c4cnco4)cc32)C1)c1ccc(Br)s1] | 5-bromo-N-(3-(6-(oxazol-5-yl)-2- (pyridin-2-yl)-1H-imidazo[4,5- c]pyridin-1-yl)cyclohexyl)thiophene- 2-carboxamide(1) |
| 4 | 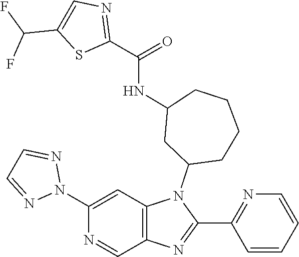 [SMILES: O=C(NC1CCCCC(n2c(-c3ccccn3)nc3cnc(-n4nccn4)cc32)C1)c1ncc(C(F)F)s1] | 5-(difluoromethyl)-N-(3-(2-(pyridin- 2-yl)-6-(2H-1,2,3-triazol-2-yl)-1H- imidazo[4,5-c]pyridin-1- yl)cycloheptyl)thiazole-2- carboxamide(2) |
| 5 | 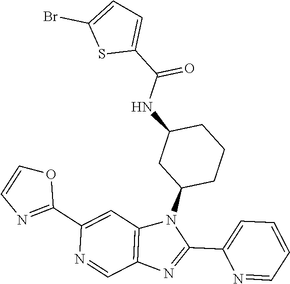 [SMILES: O=C(N[C@H]1CCC[C@@H](n2c(-c3ccccn3)nc3cnc(-c4ncco4)cc32)C1)c1ccc(Br)s1] | 5-bromo-N-((1S,3R)-3-(6-(oxazol-2- yl)-2-(pyridin-2-yl)-1H-imidazo[4,5- c]pyridin-1-yl)cyclohexyl)thiophene- 2-carboxamide |
| 6 | 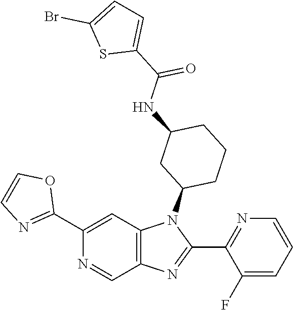 [SMILES: O=C(N[C@H]1CCC[C@@H](n2c(-c3ncccc3F)nc3cnc(-c4ncco4)cc32)C1)c1ccc(Br)s1] | 5-bromo-N-((1S,3R)-3-(2-(3- fluoropyridin-2-yl)-6-(oxazol-2-yl)- 1H-imidazo[4,5-c]pyridin-1- yl)cyclohexyl)thiophene-2- carboxamide |
| 7 | 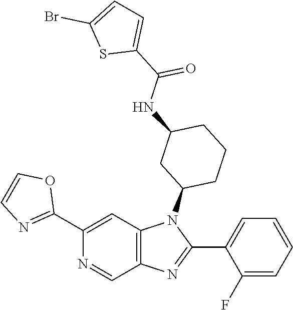 [SMILES: O=C(N[C@H]1CCC[C@@H](n2c(-c3ccccc3F)nc3cnc(-c4ncco4)cc32)C1)c1ccc(Br)s1] | 5-bromo-N-((1S,3R)-3-(2-(2- fluorophenyl)-6-(oxazol-2-yl)-1H- imidazo[4,5-c]pyridin-1- yl)cyclohexyl)thiophene-2- carboxamide |
| 8 | 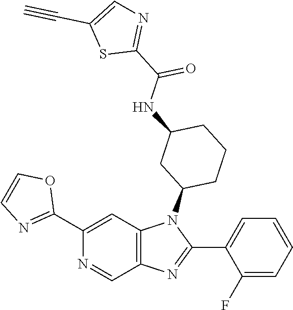 [SMILES: C#Cc1cnc(C(=O)N[C@H]2CCC[C@@H](n3c(-c4ccccc4F)nc4cnc(-c5ncco5)cc43)C2)s1] | 5-ethynyl-N-((1S,3R)-3-(2-(2- fluorophenyl)-6-(oxazol-2-yl)-1H- imidazo[4,5-c]pyridin-1- yl)cyclohexyl)thiazole-2-carboxamide |
| 9 | 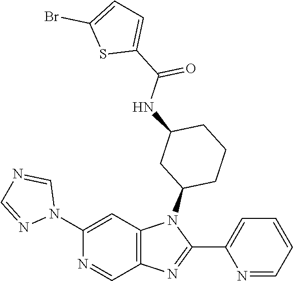 [SMILES: O=C(N[C@H]1CCC[C@@H](n2c(-c3ccccn3)nc3cnc(-n4cncn4)cc32)C1)c1ccc(Br)s1] | 5-bromo-N-((1S,3R)-3-(2-(pyridin-2- yl)-6-(1H-1,2,4-triazol-1-yl)-1H- imidazo[4,5-c]pyridin-1- yl)cyclohexyl)thiophene-2- carboxamide |
| 10 | 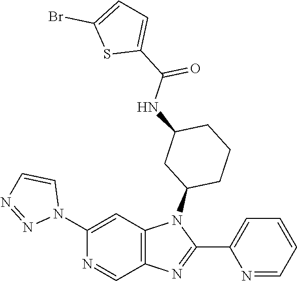 [SMILES: O=C(N[C@H]1CCC[C@@H](n2c(-c3ccccn3)nc3cnc(-n4ccnn4)cc32)C1)c1ccc(Br)s1] | 5-bromo-N-((1S,3R)-3-(2-(pyridin-2- yl)-6-(1H-1,2,3-triazol-1-yl)-1H- imidazo[4,5-c]pyridin-1- yl)cyclohexyl)thiophene-2- carboxamide |
| 11 | 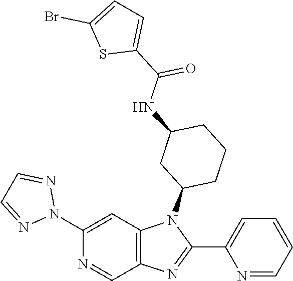 [SMILES: O=C(N[C@H]1CCC[C@@H](n2c(-c3ccccn3)nc3cnc(-n4nccn4)cc32)C1)c1ccc(Br)s1] | 5-bromo-N-((1S,3R)-3-(2-(pyridin-2- yl)-6-(2H-1,2,3-triazol-2-yl)-1H- imidazo[4,5-c]pyridin-1- yl)cyclohexyl)thiophene-2- carboxamide |
| 12 | 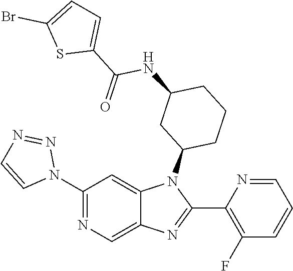 [SMILES: O=C(N[C@H]1CCC[C@@H](n2c(-c3ncccc3F)nc3cnc(-n4ccnn4)cc32)C1)c1ccc(Br)s1] | 5-bromo-N-((1S,3R)-3-(2-(3- fluoropyridin-2-yl)-6-(1H-1,2,3- triazol-1-yl)-1H-imidazo[4,5- c]pyridin-1-yl)cyclohexyl)thiophene- 2-carboxamide |
| 13 | 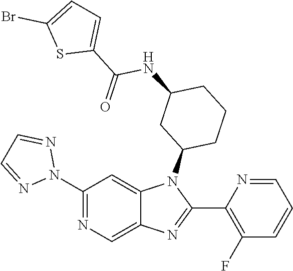 [SMILES: O=C(N[C@H]1CCC[C@@H](n2c(-c3ncccc3F)nc3cnc(-n4nccn4)cc32)C1)c1ccc(Br)s1] | 5-bromo-N-((1S,3R)-3-(2-(3- fluoropyridin-2-yl)-6-(2H-1,2,3- triazol-2-yl)-1H-imidazo[4,5- c]pyridin-1-yl)cyclohexyl)thiophene- 2-carboxamide |
| 14 | 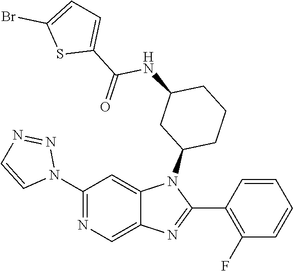 [SMILES: O=C(N[C@H]1CCC[C@@H](n2c(-c3ccccc3F)nc3cnc(-n4ccnn4)cc32)C1)c1ccc(Br)s1] | 5-bromo-N-((1S,3R)-3-(2-(2- fluorophenyl)-6-(1H-1,2,3-triazol-1- yl)-1H-imidazo[4,5-c]pyridin-1- yl)cyclohexyl)thiophene-2- carboxamide |
| 15 | 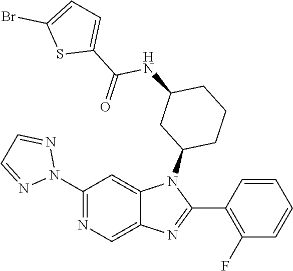 [SMILES: O=C(N[C@H]1CCC[C@@H](n2c(-c3ccccc3F)nc3cnc(-n4nccn4)cc32)C1)c1ccc(Br)s1] | 5-bromo-N-((1S,3R)-3-(2-(2- fluorophenyl)-6-(2H-1,2,3-triazol-2- yl)-1H-imidazo[4,5-c]pyridin-1- yl)cyclohexyl)thiophene-2- carboxamide |
| 16 | 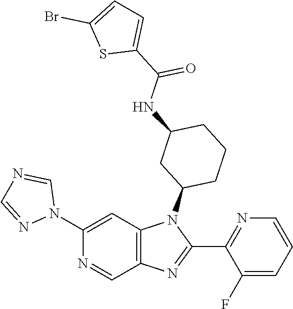 [SMILES: O=C(N[C@H]1CCC[C@@H](n2c(-c3ncccc3F)nc3cnc(-n4cncn4)cc32)C1)c1ccc(Br)s1] | 5-bromo-N-((1S,3R)-3-(2-(3- fluoropyridin-2-yl)-6-(1H-1,2,4- triazol-1-yl)-1H-imidazo[4,5- c]pyridin-1-yl)cyclohexyl)thiophene- 2-carboxamide |
| 17 | 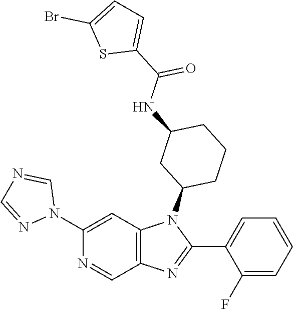 [SMILES: O=C(N[C@H]1CCC[C@@H](n2c(-c3ccccc3F)nc3cnc(-n4cncn4)cc32)C1)c1ccc(Br)s1] | 5-bromo-N-((1S,3R)-3-(2-(2- fluorophenyl)-6-(1H-1,2,4-triazol-1- yl)-1H-imidazo[4,5-c]pyridin-1- yl)cyclohexyl)thiophene-2- carboxamide |
| 18 | 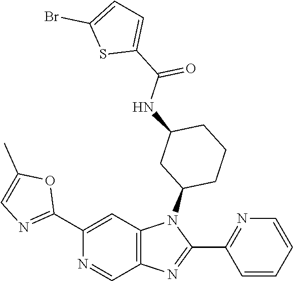 [SMILES: Cc1cnc(-c2cc3c(cn2)nc(-c2ccccn2)n3[C@@H]2CCC[C@H](NC(=O)c3ccc(Br)s3)C2)o1] | 5-bromo-N-((1S,3R)-3-(6-(5- methyloxazol-2-yl)-2-(pyridin-2-yl)- 1H-imidazo[4,5-c]pyridin-1- yl)cyclohexyl)thiophene-2- carboxamide |
| 19 | 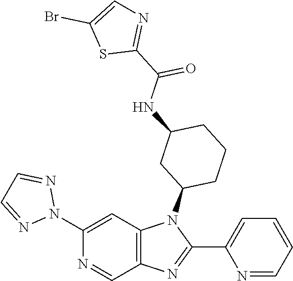 [SMILES: O=C(N[C@H]1CCC[C@@H](n2c(-c3ccccn3)nc3cnc(-n4nccn4)cc32)C1)c1ncc(Br)s1] | 5-bromo-N-((1S,3R)-3-(2-(pyridin-2- yl)-6-(2H-1,2,3-triazol-2-yl)-1H- imidazo[4,5-c]pyridin-1- yl)cyclohexyl)thiazole-2-carboxamide |
| 20 | 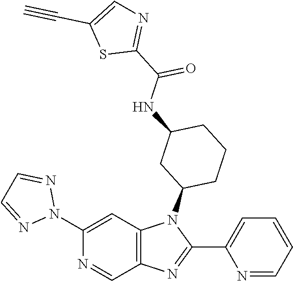 [SMILES: C#Cc1cnc(C(=O)N[C@H]2CCC[C@@H](n3c(-c4ccccn4)nc4cnc(-n5nccn5)cc43)C2)s1] | 5-ethynyl-N-((1S,3R)-3-(2-(pyridin- 2-yl)-6-(2H-1,2,3-triazol-2-yl)-1H- imidazo[4,5-c]pyridin-1- yl)cyclohexyl)thiazole-2-carboxamide |
| 21 | 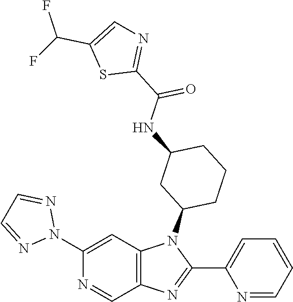 [SMILES: O=C(N[C@H]1CCC[C@@H](n2c(-c3ccccn3)nc3cnc(-n4nccn4)cc32)C1)c1ncc(C(F)F)s1] | 5-(difluoromethyl)-N-((1S,3R)-3-(2- (pyridin-2-yl)-6-(2H-1,2,3-triazol-2- yl)-1H-imidazo[4,5-c]pyridin-1- yl)cyclohexyl)thiazole-2-carboxamide |
| 22 | 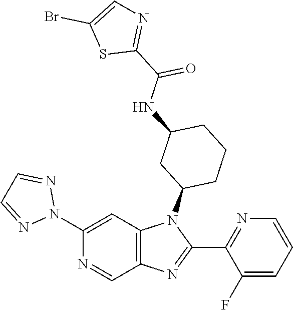 [SMILES: O=C(N[C@H]1CCC[C@@H](n2c(-c3ncccc3F)nc3cnc(-n4nccn4)cc32)C1)c1ncc(Br)s1] | 5-bromo-N-((1S,3R)-3-(2-(3- fluoropyridin-2-yl)-6-(2H-1,2,3- triazol-2-yl)-1H-imidazo[4,5- c]pyridin-1-yl)cyclohexyl)thiazole-2- carboxamide |
| 23 | 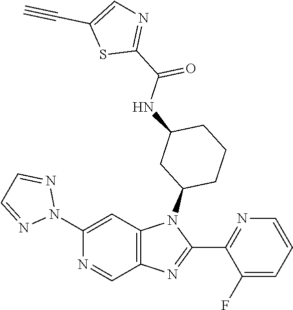 [SMILES: C#Cc1cnc(C(=O)N[C@H]2CCC[C@@H](n3c(-c4ncccc4F)nc4cnc(-n5nccn5)cc43)C2)s1] | 5-ethynyl-N-((1S,3R)-3-(2-(3- fluoropyridin-2-yl)-6-(2H-1,2,3- triazol-2-yl)-1H-imidazo[4,5- c]pyridin-1-yl)cyclohexyl)thiazole-2- carboxamide |
| 24 | 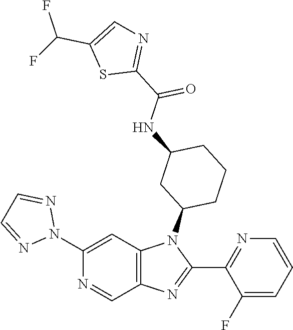 [SMILES: O=C(N[C@H]1CCC[C@@H](n2c(-c3ncccc3F)nc3cnc(-n4nccn4)cc32)C1)c1ncc(C(F)F)s1] | 5-(difluoromethyl)-N-((1S,3R)-3-(2- (3-fluoropyridin-2-yl)-6-(2H-1,2,3- triazol-2-yl)-1H-imidazo[4,5- c]pyridin-1-yl)cyclohexyl)thiazole-2- carboxamide |
| 25 | 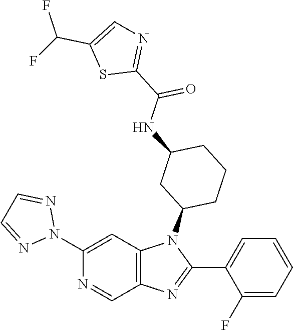 [SMILES: O=C(N[C@H]1CCC[C@@H](n2c(-c3ccccc3F)nc3cnc(-n4nccn4)cc32)C1)c1ncc(C(F)F)s1] | 5-(difluoromethyl)-N-((1S,3R)-3-(2- (2-fluorophenyl)-6-(2H-1,2,3-triazol- 2-yl)-1H-imidazo[4,5-c]pyridin-1- yl)cyclohexyl)thiazole-2-carboxamide |
| 26 | 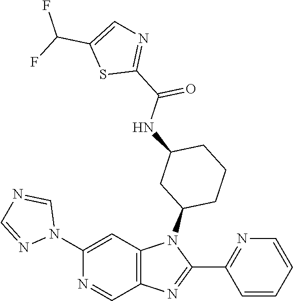 [SMILES: O=C(N[C@H]1CCC[C@@H](n2c(-c3ccccn3)nc3cnc(-n4cncn4)cc32)C1)c1ncc(C(F)F)s1] | 5-(difluoromethyl)-N-((1S,3R)-3-(2- (pyridin-2-yl)-6-(1H-1,2,4-triazol-1- yl)-1H-imidazo[4,5-c]pyridin-1- yl)cyclohexyl)thiazole-2-carboxamide |
| 27 | 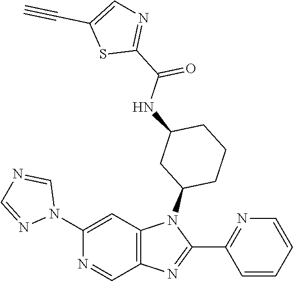 [SMILES: C#Cc1cnc(C(=O)N[C@H]2CCC[C@@H](n3c(-c4ccccn4)nc4cnc(-n5cncn5)cc43)C2)s1] | 5-ethynyl-N-((1S,3R)-3-(2-(pyridin- 2-yl)-6-(1H-1,2,4-triazol-1-yl)-1H- imidazo[4,5-c]pyridin-1- yl)cyclohexyl)thiazole-2-carboxamide |
| 28 | 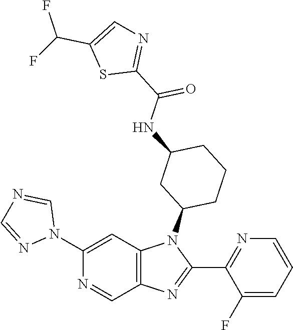 [SMILES: O=C(N[C@H]1CCC[C@@H](n2c(-c3ncccc3F)nc3cnc(-n4cncn4)cc32)C1)c1ncc(C(F)F)s1] | 5-(difluoromethyl)-N-((1S,3R)-3-(2- (3-fluoropyridin-2-yl)-6-(1H-1,2,4- triazol-1-yl)-1H-imidazo[4,5- c]pyridin-1-yl)cyclohexyl)thiazole-2- carboxamide |
| 29 | 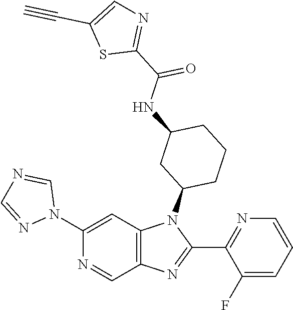 [SMILES: C#Cc1cnc(C(=O)N[C@H]2CCC[C@@H](n3c(-c4ncccc4F)nc4cnc(-n5cncn5)cc43)C2)s1] | 5-ethynyl-N-((1S,3R)-3-(2-(3- fluoropyridin-2-yl)-6-(1H-1,2,4- triazol-1-yl)-1H-imidazo[4,5- c]pyridin-1-yl)cyclohexyl)thiazole-2- carboxamide |
| 30 | 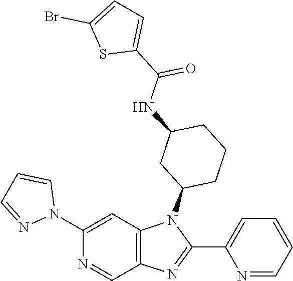 [SMILES: O=C(N[C@H]1CCC[C@@H](n2c(-c3ccccn3)nc3cnc(-n4cccn4)cc32)C1)c1ccc(Br)s1] | N-((1S,3R)-3-(6-(1H-pyrazol-1-yl)-2- (pyridin-2-yl)-1H-imidazo[4,5- c]pyridin-1-yl)cyclohexyl)-5- bromothiophene-2-carboxamide |
| 31 | 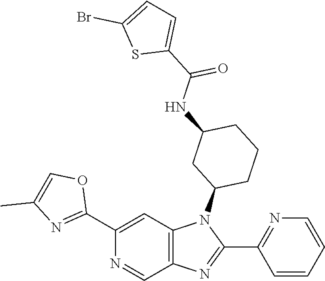 [SMILES: Cc1coc(-c2cc3c(cn2)nc(-c2ccccn2)n3[C@@H]2CCC[C@H](NC(=O)c3ccc(Br)s3)C2)n1] | 5-bromo-N-((1S,3R)-3-(6-(4- methyloxazol-2-yl)-2-(pyridin-2-yl)- 1H-imidazo[4,5-c]pyridin-1- yl)cyclohexyl)thiophene-2- carboxamide |
| 32 | 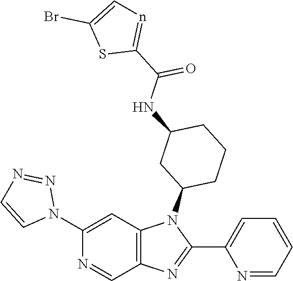 | 5-bromo-N-((1S,3R)-3-(2-(pyridin-2- yl)-6-(1H-1,2,3-triazol-1-yl)-1H- imidazo[4,5-c]pyridin-1- yl)cyclohexyl)thiazole-2-carboxamide |
| 33 | 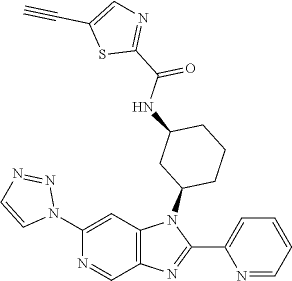 [SMILES: C#Cc1cnc(C(=O)N[C@H]2CCC[C@@H](n3c(-c4ccccn4)nc4cnc(-n5ccnn5)cc43)C2)s1] | 5-ethynyl-N-((1S,3R)-3-(2-(pyridin- 2-yl)-6-(1H-1,2,3-triazol-1-yl)-1H- imidazo[4,5-c]pyridin-1- yl)cyclohexyl)thiazole-2-carboxamide |
| 34 | 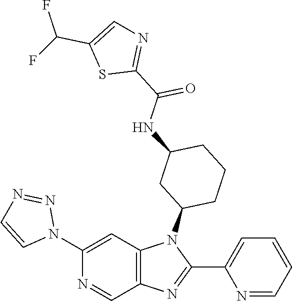 [SMILES: O=C(N[C@H]1CCC[C@@H](n2c(-c3ccccn3)nc3cnc(-n4ccnn4)cc32)C1)c1ncc(C(F)F)s1] | 5-(difluoromethyl)-N-((1S,3R)-3-(2- (pyridin-2-yl)-6-(1H-1,2,3-triazol-1- yl)-1H-imidazo[4,5-c]pyridin-1- yl)cyclohexyl)thiazole-2-carboxamide |
| 35 | 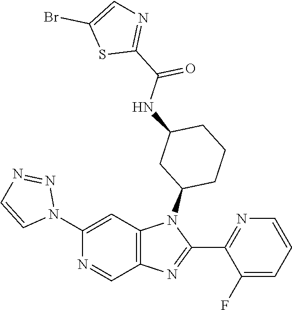 [SMILES: O=C(N[C@H]1CCC[C@@H](n2c(-c3ncccc3F)nc3cnc(-n4ccnn4)cc32)C1)c1ncc(Br)s1] | 5-bromo-N-((1S,3R)-3-(2-(3- fluoropyridin-2-yl)-6-(1H-1,2,3- triazol-1-yl)-1H-imidazo[4,5- c]pyridin-1-yl)cyclohexyl)thiazole-2- carboxamide |
| 36 | 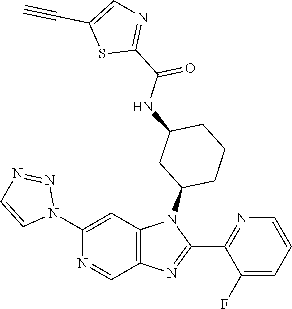 [SMILES: C#Cc1cnc(C(=O)N[C@H]2CCC[C@@H](n3c(-c4ncccc4F)nc4cnc(-n5ccnn5)cc43)C2)s1] | 5-ethynyl-N-((1S,3R)-3-(2-(3- fluoropyridin-2-yl)-6-(1H-1,2,3- triazol-1-yl)-1H-imidazo[4,5- c]pyridin-1-yl)cyclohexyl)thiazole-2- carboxamide |
| 37 | 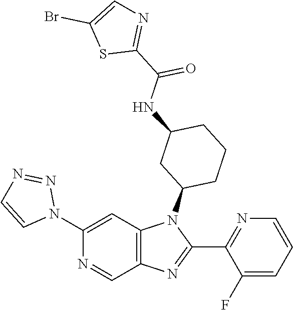 [SMILES: O=C(N[C@H]1CCC[C@@H](n2c(-c3ncccc3F)nc3cnc(-n4ccnn4)cc32)C1)c1ncc(Br)s1] | 5-(difluoromethyl)-N-((1S,3R)-3-(2- (3-fluoropyridin-2-yl)-6-(1H-1,2,3- triazol-1-yl)-1H-imidazo[4,5- c]pyridin-1-yl)cyclohexyl)thiazole-2- carboxamide |
| 38 | 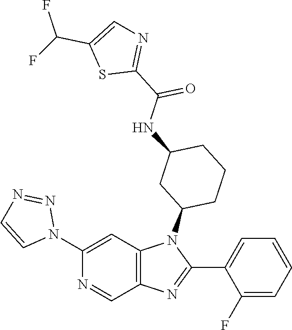 [SMILES: O=C(N[C@H]1CCC[C@@H](n2c(-c3ccccc3F)nc3cnc(-n4ccnn4)cc32)C1)c1ncc(C(F)F)s1] | 5-(difluoromethyl)-N-((1S,3R)-3-(2- (2-fluorophenyl)-6-(1H-1,2,3-triazol- 1-yl)-1H-imidazo[4,5-c]pyridin-1- yl)cyclohexyl)thiazole-2-carboxamide |
| 39 | 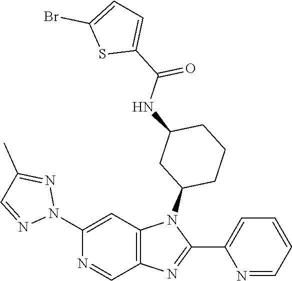 [SMILES: Cc1cnn(-c2cc3c(cn2)nc(-c2ccccn2)n3[C@@H]2CCC[C@H](NC(=O)c3ccc(Br)s3)C2)n1] | 5-bromo-N-((1S,3R)-3-(6-(4-methyl- 2H-1,2,3-triazol-2-yl)-2-(pyridin-2- yl)-1H-imidazo[4,5-c]pyridin-1- yl)cyclohexyl)thiophene-2- carboxamide |
| 40 | 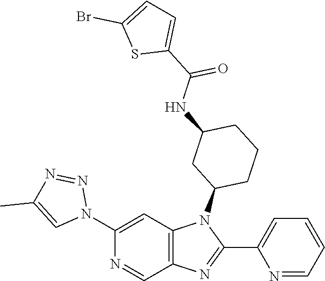 [SMILES: Cc1cn(-c2cc3c(cn2)nc(-c2ccccn2)n3[C@@H]2CCC[C@H](NC(=O)c3ccc(Br)s3)C2)nn1] | 5-bromo-N-((1S,3R)-3-(6-(4-methyl- 1H-1,2,3-triazol-1-yl)-2-(pyridin-2- yl)-1H-imidazo[4,5-c]pyridin-1- yl)cyclohexyl)thiophene-2- carboxamide |
| 41 | 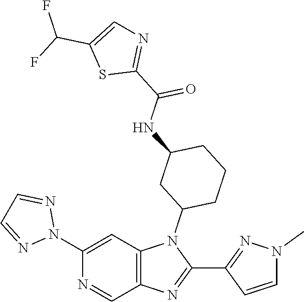 [SMILES: Cn1ccc(-c2nc3cnc(-n4nccn4)cc3n2C2CCC[C@H](NC(=O)c3ncc(C(F)F)s3)C2)n1] | 5-(difluoromethyl)-N-((1S,3R)-3-(2- (1-methyl-1H-pyrazol-3-yl)-6-(2H- 1,2,3-triazol-2-yl)-1H-imidazo[4,5- c]pyridin-1-yl)cyclohexyl)thiazole-2- carboxamide |
| 42 | 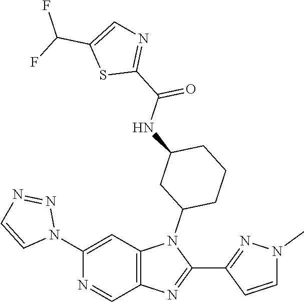 [SMILES: Cn1ccc(-c2nc3cnc(-n4ccnn4)cc3n2C2CCC[C@H](NC(=O)c3ncc(C(F)F)s3)C2)n1] | 5-(difluoromethyl)-N-((1S,3R)-3-(2- (1-methyl-1H-pyrazol-3-yl)-6-(1H- 1,2,3-triazol-1-yl)-1H-imidazo[4,5- c]pyridin-1-yl)cyclohexyl)thiazole-2- carboxamide |
| 43 | 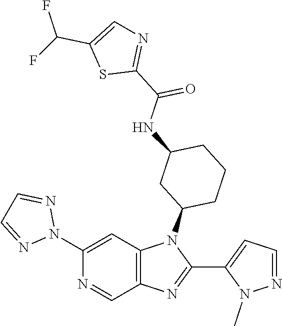 [SMILES: Cn1nccc1-c1nc2cnc(-n3nccn3)cc2n1[C@@H]1CCC[C@H](NC(=O)c2ncc(C(F)F)s2)C1] | 5-(difluoromethyl)-N-((1S,3R)-3-(2- (1-methyl-1H-pyrazol-5-yl)-6-(2H- 1,2,3-triazol-2-yl)-1H-imidazo[4,5- c]pyridin-1-yl)cyclohexyl)thiazole-2- carboxamide |
| 44 | 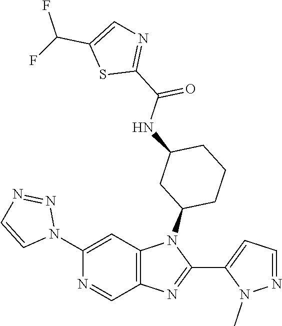 [SMILES: Cn1nccc1-c1nc2cnc(-n3ccnn3)cc2n1[C@@H]1CCC[C@H](NC(=O)c2ncc(C(F)F)s2)C1] | 5-(difluoromethyl)-N-((1S,3R)-3-(2- (1-methyl-1H-pyrazol-5-yl)-6-(1H- 1,2,3-triazol-1-yl)-1H-imidazo[4,5- c]pyridin-1-yl)cyclohexyl)thiazole-2- carboxamide |
| 45 | 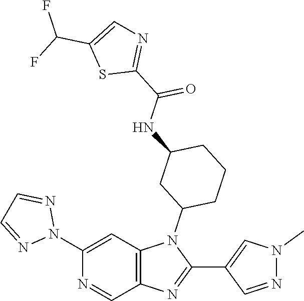 [SMILES: Cn1cc(-c2nc3cnc(-n4nccn4)cc3n2C2CCC[C@H](NC(=O)c3ncc(C(F)F)s3)C2)cn1] | 5-(difluoromethyl)-N-((1S,3R)-3-(2- (1-methyl-1H-pyrazol-4-yl)-6-(2H- 1,2,3-triazol-2-yl)-1H-imidazo[4,5- c]pyridin-1-yl)cyclohexyl)thiazole-2- carboxamide |
| 46 | 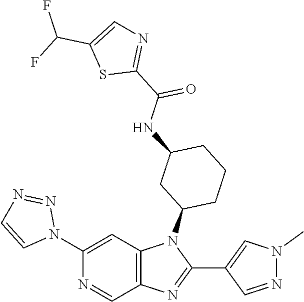 [SMILES: Cn1cc(-c2nc3cnc(-n4ccnn4)cc3n2[C@@H]2CCC[C@H](NC(=O)c3ncc(C(F)F)s3)C2)cn1] | 5-(difluoromethyl)-N-((1S,3R)-3-(2- (1-methyl-1H-pyrazol-4-yl)-6-(1H- 1,2,3-triazol-1-yl)-1H-imidazo[4,5- c]pyridin-1-yl)cyclohexyl)thiazole-2- carboxamide |
| 47 | 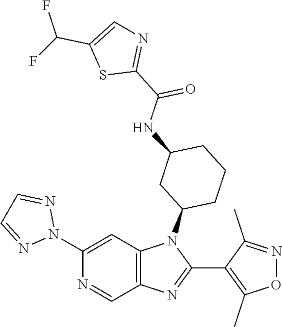 [SMILES: Cc1noc(C)c1-c1nc2cnc(-n3nccn3)cc2n1[C@@H]1CCC[C@H](NC(=O)c2ncc(C(F)F)s2)C1] | 5-(difluoromethyl)-N-((1S,3R)-3-(2- (3,5-dimethylisoxazol-4-yl)-6-(2H- 1,2,3-triazol-2-yl)-1H-imidazo[4,5- c]pyridin-1-yl)cyclohexyl)thiazole-2- carboxamide |
| 48 | 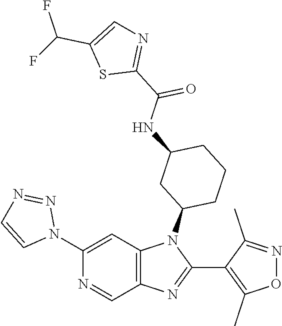 [SMILES: Cc1noc(C)c1-c1nc2cnc(-n3ccnn3)cc2n1[C@@H]1CCC[C@H](NC(=O)c2ncc(C(F)F)s2)C1] | 5-(difluoromethyl)-N-((1S,3R)-3-(2- (3,5-dimethylisoxazol-4-yl)-6-(1H- 1,2,3-triazol-1-yl)-1H-imidazo[4,5- c]pyridin-1-yl)cyclohexyl)thiazole-2- carboxamide |
| 49 | 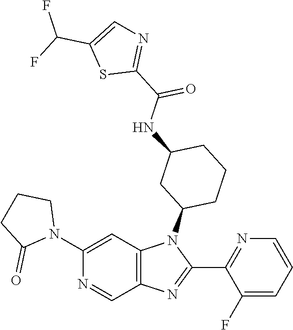 [SMILES: O=C(N[C@H]1CCC[C@@H](n2c(-c3ncccc3F)nc3cnc(N4CCCC4=O)cc32)C1)c1ncc(C(F)F)s1] | 5-(difluoromethyl)-N-((1S,3R)-3-(2- (3-fluoropyridin-2-yl)-6-(2- oxopyrrolidin-1-yl)-1H-imidazo[4,5- c]pyridin-1-yl)cyclohexyl)thiazole-2- carboxamide |
| 50 | 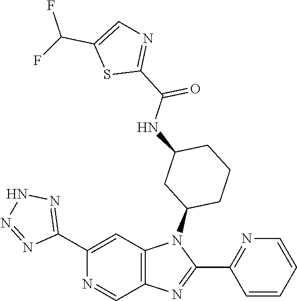 [SMILES: O=C(N[C@H]1CCC[C@@H](n2c(-c3ccccn3)nc3cnc(-c4nn[nH]n4)cc32)C1)c1ncc(C(F)F)s1] | 5-(difluoromethyl)-N-((1S,3R)-3-(2- (pyridin-2-yl)-6-(2H-tetrazol-5-yl)- 1H-imidazo[4,5-c]pyridin-1- yl)cyclohexyl)thiazole-2-carboxamide |
| 51 | 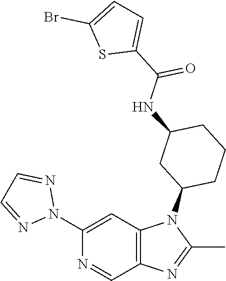 [SMILES: Cc1nc2cnc(-n3nccn3)cc2n1[C@@H]1CCC[C@H](NC(=O)c2ccc(Br)s2)C1] | 5-bromo-N-((1S,3R)-3-(2-methyl-6- (2H-1,2,3-triazol-2-yl)-1H- imidazo[4,5-c]pyridin-1- yl)cyclohexyl)thiophene-2- carboxamide |
| 52 | 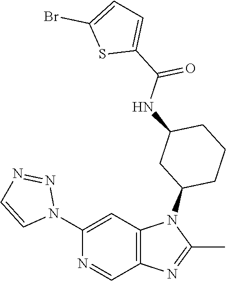 [SMILES: Cc1nc2cnc(-n3ccnn3)cc2n1[C@@H]1CCC[C@H](NC(=O)c2ccc(Br)s2)C1] | 5-bromo-N-((1S,3R)-3-(2-methyl-6- (1H-1,2,3-triazol-1-yl)-1H- imidazo[4,5-c]pyridin-1- yl)cyclohexyl)thiophene-2- carboxamide |
| 53 | 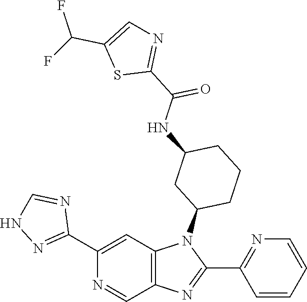 [SMILES: O=C(N[C@H]1CCC[C@@H](n2c(-c3ccccn3)nc3cnc(-c4nc[nH]n4)cc32)C1)c1ncc(C(F)F)s1] | 5-(difluoromethyl)-N-((1S,3R)-3-(2- (pyridin-2-yl)-6-(1H-1,2,4-triazol-3- yl)-1H-imidazo[4,5-c]pyridin-1- yl)cyclohexyl)thiazole-2-carboxamide |
| 54 | 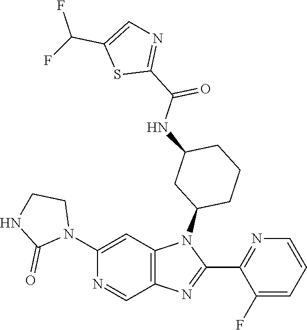 [SMILES: O=C(N[C@H]1CCC[C@@H](n2c(-c3ncccc3F)nc3cnc(N4CCNC4=O)cc32)C1)c1ncc(C(F)F)s1] | 5-(difluoromethyl)-N-((1S,3R)-3-(2- (3-fluoropyridin-2-yl)-6-(2- oxoimidazolidin-1-yl)-1H- imidazo[4,5-c]pyridin-1- yl)cyclohexyl)thiazole-2-carboxamide |
| 55 | 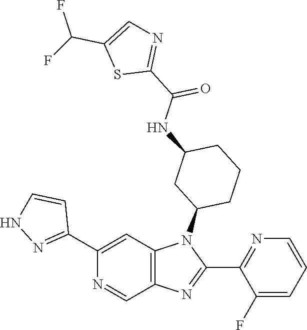 [SMILES: O=C(N[C@H]1CCC[C@@H](n2c(-c3ncccc3F)nc3cnc(-c4cc[nH]n4)cc32)C1)c1ncc(C(F)F)s1] | 5-(difluoromethyl)-N-((1S,3R)-3-(2- (3-fluoropyridin-2-yl)-6-(1H-pyrazol- 3-yl)-1H-imidazo[4,5-c]pyridin-1- yl)cyclohexyl)thiazole-2-carboxamide |
| 56 | 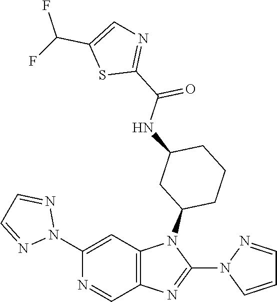 [SMILES: O=C(N[C@H]1CCC[C@@H](n2c(-n3cccn3)nc3cnc(-n4nccn4)cc32)C1)c1ncc(C(F)F)s1] | N-((1S,3R)-3-(2-(1H-pyrazol-1-yl)-6- (2H-1,2,3-triazol-2-yl)-1H- imidazo[4,5-c]pyridin-1- yl)cyclohexyl)-5- (difluoromethyl)thiazole-2- carboxamide |
| 57 | 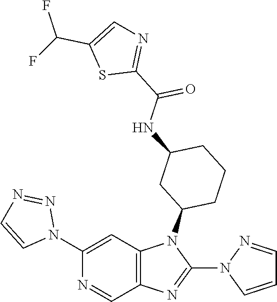 [SMILES: O=C(N[C@H]1CCC[C@@H](n2c(-n3cccn3)nc3cnc(-n4ccnn4)cc32)C1)c1ncc(C(F)F)s1] | N-((1S,3R)-3-(2-(1H-pyrazol-1-yl)-6- (1H-1,2,3-triazol-1-yl)-1H- imidazo[4,5-c]pyridin-1- yl)cyclohexyl)-5- (difluoromethyl)thiazole-2- carboxamide |
| 58 | 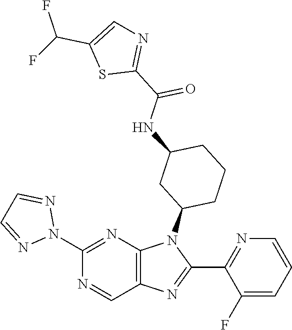 [SMILES: O=C(N[C@H]1CCC[C@@H](n2c(-c3ncccc3F)nc3cnc(-n4nccn4)nc32)C1)c1ncc(C(F)F)s1] | 5-(difluoromethyl)-N-((1S,3R)-3-(8- (3-fluoropyridin-2-yl)-2-(2H-1,2,3- triazol-2-yl)-9H-purin-9- yl)cyclohexyl)thiazole-2-carboxamide |
| 59 | 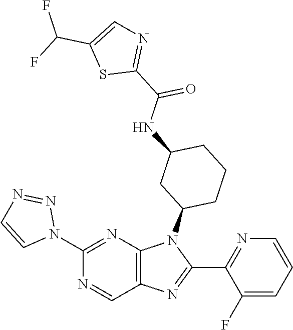 [SMILES: O=C(N[C@H]1CCC[C@@H](n2c(-c3ncccc3F)nc3cnc(-n4ccnn4)nc32)C1)c1ncc(C(F)F)s1] | 5-(difluoromethyl)-N-((1S,3R)-3-(8- (3-fluoropyridin-2-yl)-2-(1H-1,2,3- triazol-1-yl)-9H-purin-9- yl)cyclohexyl)thiazole-2-carboxamide |
| 60 | 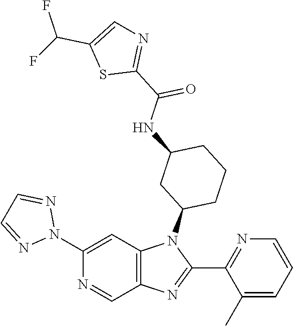 [SMILES: Cc1cccnc1-c1nc2cnc(-n3nccn3)cc2n1[C@@H]1CCC[C@H](NC(=O)c2ncc(C(F)F)s2)C1] | 5-(difluoromethyl)-N-((1S,3R)-3-(2- (3-methylpyridin-2-yl)-6-(2H-1,2,3- triazol-2-yl)-1H-imidazo[4,5- c]pyridin-1-yl)cyclohexyl)thiazole-2- carboxamide |
| 61 | 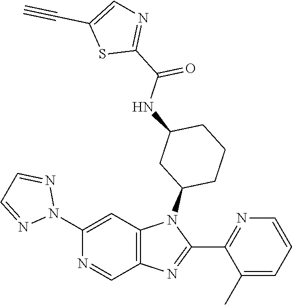 [SMILES: C#Cc1cnc(C(=O)N[C@H]2CCC[C@@H](n3c(-c4ncccc4C)nc4cnc(-n5nccn5)cc43)C2)s1] | 5-ethynyl-N-((1S,3R)-3-(2-(3- methylpyridin-2-yl)-6-(2H-1,2,3- triazol-2-yl)-1H-imidazo[4,5- c]pyridin-1-yl)cyclohexyl)thiazole-2- carboxamide |
| 62 | 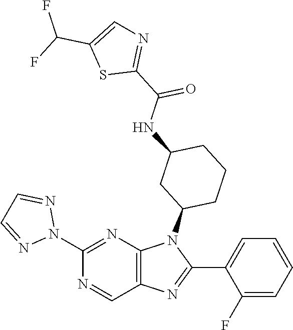 [SMILES: O=C(N[C@H]1CCC[C@@H](n2c(-c3ccccc3F)nc3cnc(-n4nccn4)nc32)C1)c1ncc(C(F)F)s1] | 5-(difluoromethyl)-N-((1S,3R)-3-(8- (2-fluorophenyl)-2-(2H-1,2,3-triazol- 2-yl)-9H-purin-9- yl)cyclohexyl)thiazole-2-carboxamide |
| 63 | 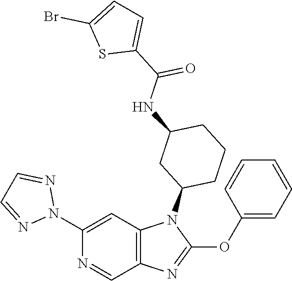 [SMILES: O=C(N[C@H]1CCC[C@@H](n2c(Oc3ccccc3)nc3cnc(-n4nccn4)cc32)C1)c1ccc(Br)s1] | 5-bromo-N-((1S,3R)-3-(2-phenoxy-6- (2H-1,2,3-triazol-2-yl)-1H- imidazo[4,5-c]pyridin-1- yl)cyclohexyl)thiophene-2- carboxamide |
| 64 | 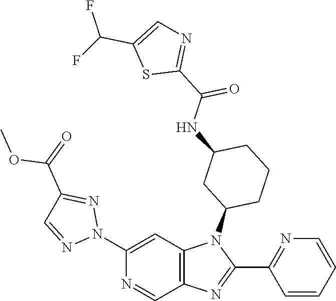 [SMILES: COC(=O)c1cnn(-c2cc3c(cn2)nc(-c2ccccn2)n3[C@@H]2CCC[C@H](NC(=O)c3ncc(C(F)F)s3)C2)n1] | methyl 2-(1-((1R,3S)-3-(5- (difluoromethyl)thiazole-2- carboxamido)cyclohexyl)-2-(pyridin- 2-yl)-1H-imidazo[4,5-c]pyridin-6-yl)- 2H-1,2,3-triazole-4-carboxylate |
| 65 | 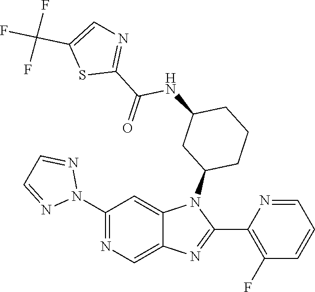 [SMILES: O=C(N[C@H]1CCC[C@@H](n2c(-c3ncccc3F)nc3cnc(-n4nccn4)cc32)C1)c1ncc(C(F)(F)F)s1] | N-((1S,3R)-3-(2-(3-fluoropyridin-2- yl)-6-(2H-1,2,3-triazol-2-yl)-1H- imidazo[4,5-c]pyridin-1- yl)cyclohexyl)-5- (trifluoromethyl)thiazole-2- carboxamide |
| 66 | 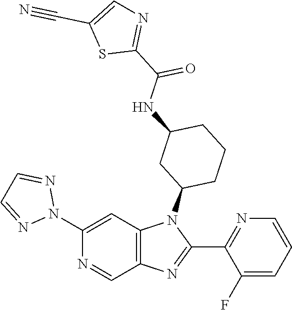 [SMILES: N#Cc1cnc(C(=O)N[C@H]2CCC[C@@H](n3c(-c4ncccc4F)nc4cnc(-n5nccn5)cc43)C2)s1] | 5-cyano-N-((1S,3R)-3-(2-(3- fluoropyridin-2-yl)-6-(2H-1,2,3- triazol-2-yl)-1H-imidazo[4,5- c]pyridin-1-yl)cyclohexyl)thiazole-2- carboxamide |
| 67 | 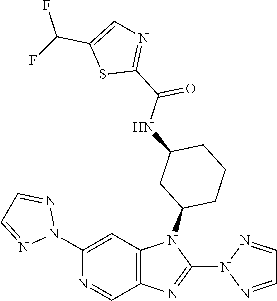 [SMILES: O=C(N[C@H]1CCC[C@@H](n2c(-n3nccn3)nc3cnc(-n4nccn4)cc32)C1)c1ncc(C(F)F)s1] | N-((1S,3R)-3-(2,6-di(2H-1,2,3- triazol-2-yl)-1H-imidazo[4,5- c]pyridin-1-yl)cyclohexyl)-5- (difluoromethyl)thiazole-2- carboxamide |
| 68 | 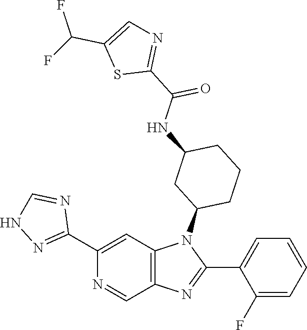 [SMILES: O=C(N[C@H]1CCC[C@@H](n2c(-c3ccccc3F)nc3cnc(-c4nc[nH]n4)cc32)C1)c1ncc(C(F)F)s1] | 5-(difluoromethyl)-N-((1S,3R)-3-(2- (2-fluorophenyl)-6-(1H-1,2,4-triazol- 3-yl)-1H-imidazo[4,5-c]pyridin-1- yl)cyclohexyl)thiazole-2-carboxamide |
| 69 | 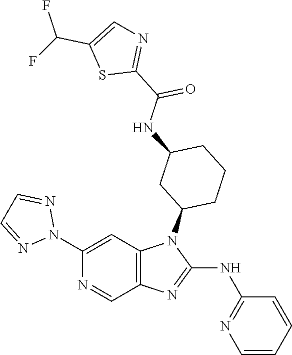 [SMILES: O=C(N[C@H]1CCC[C@@H](n2c(Nc3ccccn3)nc3cnc(-n4nccn4)cc32)C1)c1ncc(C(F)F)s1] | 5-(difluoromethyl)-N-((1S,3R)-3-(2- (pyridin-2-ylamino)-6-(2H-1,2,3- triazol-2-yl)-1H-imidazo[4,5- c]pyridin-1-yl)cyclohexyl)thiazole-2- carboxamide |
| 70 | 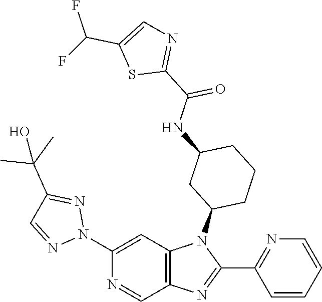 [SMILES: CC(C)(O)c1cnn(-c2cc3c(cn2)nc(-c2ccccn2)n3[C@@H]2CCC[C@H](NC(=O)c3ncc(C(F)F)s3)C2)n1] | 5-(difluoromethyl)-N-((1S,3R)-3-(6- (4-(2-hydroxypropan-2-yl)-2H-1,2,3- triazol-2-yl)-2-(pyridin-2-yl)-1H- imidazo[4,5-c]pyridin-1- yl)cyclohexyl)thiazole-2-carboxamide |
| 71 | 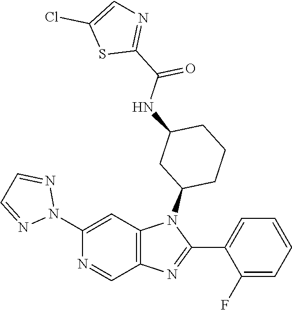 [SMILES: O=C(N[C@H]1CCC[C@@H](n2c(-c3ccccc3F)nc3cnc(-n4nccn4)cc32)C1)c1ncc(Cl)s1] | 5-chloro-N-((1S,3R)-3-(2-(2- fluorophenyl)-6-(2H-1,2,3-triazol-2- yl)-1H-imidazo[4,5-c]pyridin-1- yl)cyclohexyl)thiazole-2-carboxamide |
| 72 | 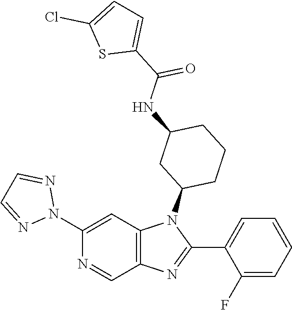 [SMILES: O=C(N[C@H]1CCC[C@@H](n2c(-c3ccccc3F)nc3cnc(-n4nccn4)cc32)C1)c1ccc(Cl)s1] | 5-chloro-N-((1S,3R)-3-(2-(2- fluorophenyl)-6-(2H-1,2,3-triazol-2- yl)-1H-imidazo[4,5-c]pyridin-1- yl)cyclohexyl)thiophene-2- carboxamide |
| 73 | 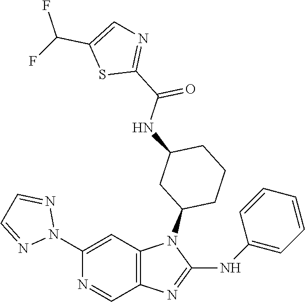 [SMILES: O=C(N[C@H]1CCC[C@@H](n2c(Nc3ccccc3)nc3cnc(-n4nccn4)cc32)C1)c1ncc(C(F)F)s1] | 5-(difluoromethyl)-N-((1S,3R)-3-(2- (phenylamino)-6-(2H-1,2,3-triazol-2- yl)-1H-imidazo[4,5-c]pyridin-1- yl)cyclohexyl)thiazole-2-carboxamide |
| 74 | 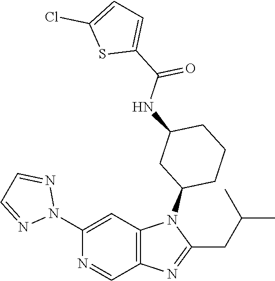 [SMILES: CC(C)Cc1nc2cnc(-n3nccn3)cc2n1[C@@H]1CCC[C@H](NC(=O)c2ccc(Cl)s2)C1] | 5-chloro-N-((1S,3R)-3-(2-isobutyl-6- (2H-1,2,3-triazol-2-yl)-1H- imidazo[4,5-c]pyridin-1- yl)cyclohexyl)thiophene-2- carboxamide |
| 75 | 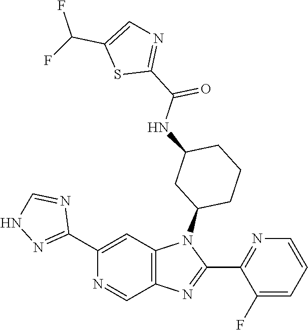 [SMILES: O=C(N[C@H]1CCC[C@@H](n2c(-c3ncccc3F)nc3cnc(-c4nc[nH]n4)cc32)C1)c1ncc(C(F)F)s1] | 5-(difluoromethyl)-N-((1S,3R)-3-(2- (3-fluoropyridin-2-yl)-6-(1H-1,2,4- triazol-3-yl)-1H-imidazo[4,5- c]pyridin-1-yl)cyclohexyl)thiazole-2- carboxamide |
| 76 | 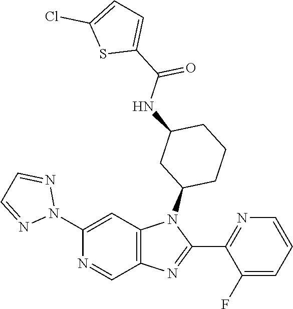 [SMILES: O=C(N[C@H]1CCC[C@@H](n2c(-c3ncccc3F)nc3cnc(-n4nccn4)cc32)C1)c1ccc(Cl)s1] | 5-chloro-N-((1S,3R)-3-(2-(3- fluoropyridin-2-yl)-6-(2H-1,2,3- triazol-2-yl)-1H-imidazo[4,5- c]pyridin-1-yl)cyclohexyl)thiophene- 2-carboxamide |
| 77 | 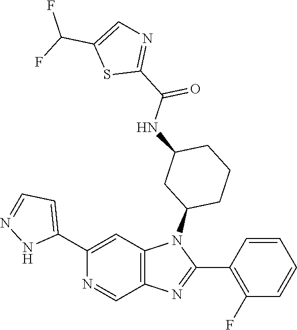 [SMILES: O=C(N[C@H]1CCC[C@@H](n2c(-c3ccccc3F)nc3cnc(-c4ccn[nH]4)cc32)C1)c1ncc(C(F)F)s1] | 5-(difluoromethyl)-N-((1S,3R)-3-(2- (2-fluorophenyl)-6-(1H-pyrazol-5-yl)- 1H-imidazo[4,5-c]pyridin-1- yl)cyclohexyl)thiazole-2-carboxamide |
| 78 | 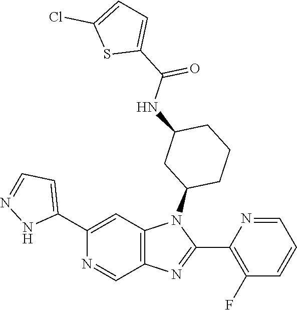 [SMILES: O=C(N[C@H]1CCC[C@@H](n2c(-c3ncccc3F)nc3cnc(-c4ccn[nH]4)cc32)C1)c1ccc(Cl)s1] | 5-chloro-N-((1S,3R)-3-(2-(3- fluoropyridin-2-yl)-6-(1H-pyrazol-5- yl)-1H-imidazo[4,5-c]pyridin-1- yl)cyclohexyl)thiophene-2- carboxamide |
| 79 | 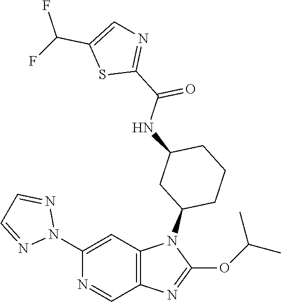 [SMILES: CC(C)Oc1nc2cnc(-n3nccn3)cc2n1[C@@H]1CCC[C@H](NC(=O)c2ncc(C(F)F)s2)C1] | 5-(difluoromethyl)-N-((1S,3R)-3-(2- isopropoxy-6-(2H-1,2,3-triazol-2-yl)- 1H-imidazo[4,5-c]pyridin-1- yl)cyclohexyl)thiazole-2-carboxamide |
| 80 | 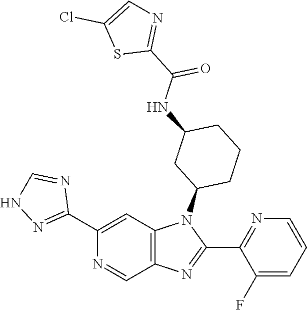 [SMILES: O=C(N[C@H]1CCC[C@@H](n2c(-c3ncccc3F)nc3cnc(-c4nc[nH]n4)cc32)C1)c1ncc(Cl)s1] | 5-chloro-N-((1S,3R)-3-(2-(3- fluoropyridin-2-yl)-6-(1H-1,2,4- triazol-3-yl)-1H-imidazo[4,5- c]pyridin-1-yl)cyclohexyl)thiophene- 2-carboxamide |
| 81 | 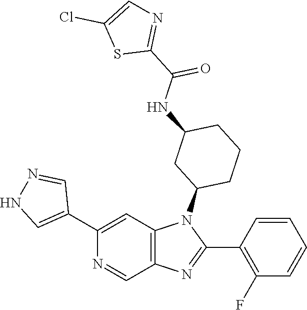 [SMILES: O=C(N[C@H]1CCC[C@@H](n2c(-c3ccccc3F)nc3cnc(-c4cn[nH]c4)cc32)C1)c1ncc(Cl)s1] | 5-chloro-N-((1S,3R)-3-(2-(2- fluorophenyl)-6-(1H-pyrazol-4-yl)- 1H-imidazo[4,5-c]pyridin-1- yl)cyclohexyl)thiazole-2-carboxamide |
| 82 | 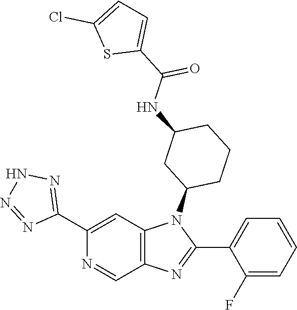 [SMILES: O=C(N[C@H]1CCC[C@@H](n2c(-c3ccccc3F)nc3cnc(-c4nn[nH]n4)cc32)C1)c1ccc(Cl)s1] | 5-chloro-N-((1S,3R)-3-(2-(2- fluorophenyl)-6-(2H-tetrazol-5-yl)- 1H-imidazo[4,5-c]pyridin-1- yl)cyclohexyl)thiophene-2- carboxamide |
| 83 | 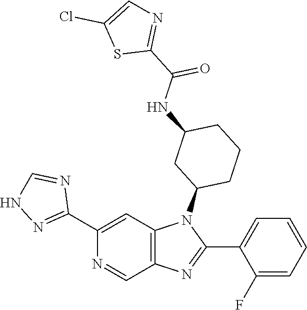 [SMILES: O=C(N[C@H]1CCC[C@@H](n2c(-c3ccccc3F)nc3cnc(-c4nc[nH]n4)cc32)C1)c1ncc(Cl)s1] | 5-chloro-N-((1S,3R)-3-(2-(2- fluorophenyl)-6-(1H-1,2,4-triazol-3- yl)-1H-imidazo[4,5-c]pyridin-1- yl)cyclohexyl)thiazole-2-carboxamide |
| 84 | 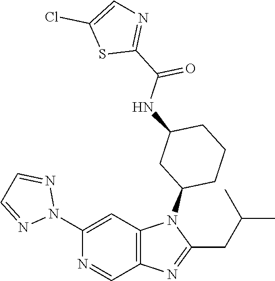 [SMILES: CC(C)Cc1nc2cnc(-n3nccn3)cc2n1[C@@H]1CCC[C@H](NC(=O)c2ncc(Cl)s2)C1] | 5-chloro-N-((1S,3R)-3-(2-isobutyl-6- (2H-1,2,3-triazol-2-yl)-1H- imidazo[4,5-c]pyridin-1- yl)cyclohexyl)thiazole-2-carboxamide |
| 85 | 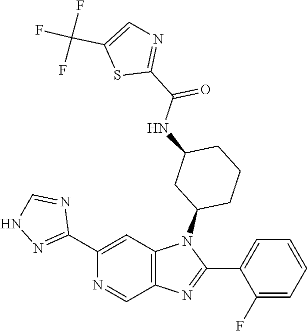 [SMILES: O=C(N[C@H]1CCC[C@@H](n2c(-c3ccccc3F)nc3cnc(-c4nc[nH]n4)cc32)C1)c1ncc(C(F)(F)F)s1] | N-((1S,3R)-3-(2-(2-fluorophenyl)-6- (1H-1,2,4-triazol-3-yl)-1H- imidazo[4,5-c]pyridin-1- yl)cyclohexyl)-5- (trifluoromethyl)thiazole-2- carboxamide |
| 86 | 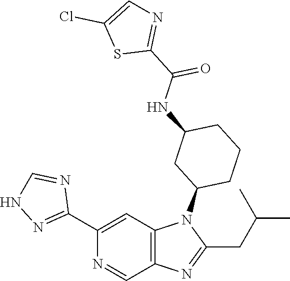 [SMILES: CC(C)Cc1nc2cnc(-c3nc[nH]n3)cc2n1[C@@H]1CCC[C@H](NC(=O)c2ncc(Cl)s2)C1] | 5-chloro-N-((1S,3R)-3-(2-isobutyl-6- (1H-1,2,4-triazol-3-yl)-1H- imidazo[4,5-c]pyridin-1- yl)cyclohexyl)thiazole-2-carboxamide |
| 87 | 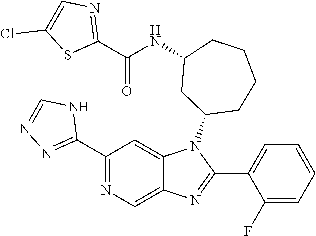 [SMILES: O=C(N[C@@H]1CCCC[C@H](n2c(-c3ccccc3F)nc3cnc(-c4nnc[nH]4)cc32)C1)c1ncc(Cl)s1] | 5-chloro-N-(3-(2-(2-fluorophenyl)-6- (4H-1,2,4-triazol-3-yl)-1H- imidazo[4,5-c]pyridin-1- yl)cycloheptyl)thiazole-2- carboxamide (Enantiomer 1) |
| 88 | 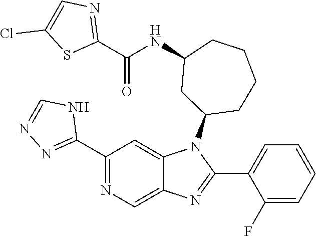 [SMILES: O=C(N[C@H]1CCCC[C@@H](n2c(-c3ccccc3F)nc3cnc(-c4nnc[nH]4)cc32)C1)c1ncc(Cl)s1] | 5-chloro-N-(3-(2-(2-fluorophenyl)-6- (4H-1,2,4-triazol-3-yl)-1H- imidazo[4,5-c]pyridin-1- yl)cycloheptyl)thiazole-2- carboxamide (Enantiomer 2) |
| 89 | 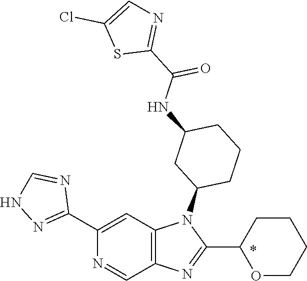 [SMILES: O=C(N[C@H]1CCC[C@@H](n2c(C3CCCCO3)nc3cnc(-c4nc[nH]n4)cc32)C1)c1ncc(Cl)s1] | 5-chloro-N-((1S,3R)-3-(2-tetrahydro- 2H-pyran-2-yl)-6-(1H-1,2,4-triazol-3- yl)-1H-imidazo[4,5-c]pyridin-1- yl)cyclohexyl)thiazole-2-carboxamide (Diastereomer 1) |
| 90 | 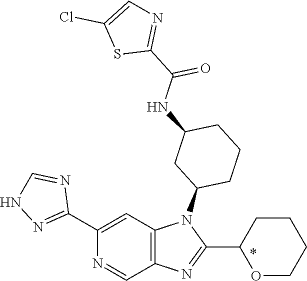 [SMILES: O=C(N[C@H]1CCC[C@@H](n2c(C3CCCCO3)nc3cnc(-c4nc[nH]n4)cc32)C1)c1ncc(Cl)s1] | 5-chloro-N-((1S,3R)-3-(2- (tetrahydro-2H-pyran-2-yl)-6-(1H- 1,2,4-triazol-3-yl)-1H-imidazo[4,5- c]pyridin-1-yl)cyclohexyl)thiazole-2- carboxamide (Diastereomer 2) |
| 91 | 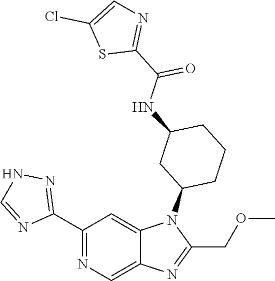 [SMILES: COCc1nc2cnc(-c3nc[nH]n3)cc2n1[C@@H]1CCC[C@H](NC(=O)c2ncc(Cl)s2)C1] | 5-chloro-N-((1S,3R)-3-(2- (methoxymethyl)-6-(1H-1,2,4-triazol- 3-yl)-1H-imidazo[4,5-c]pyridin-1- yl)cyclohexyl)thiazole-2-carboxamide |
| 92 | 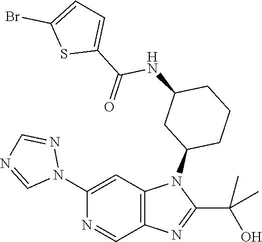 [SMILES: CC(C)(O)c1nc2cnc(-n3cncn3)cc2n1[C@@H]1CCC[C@H](NC(=O)c2ccc(Br)s2)C1] | 5-bromo-N-((1S,3R)-3-(2-(2- hydroxypropan-2-yl)-6-(1H-1,2,4- triazol-1-yl)-1H-imidazo[4,5- c]pyridin-1-yl)cyclohexyl)thiophene- 2-carboxamide |
| 93 | 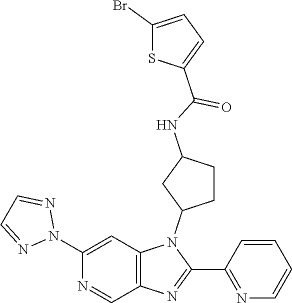 [SMILES: O=C(NC1CCC(n2c(-c3ccccn3)nc3cnc(-n4nccn4)cc32)C1)c1ccc(Br)s1] | 5-bromo-N-(3-(2-(pyridin-2-yl)-6- (2H-1,2,3-triazol-2-yl)-1H- imidazo[4,5-c]pyridin-1- yl)cyclopentyl)thiophene-2- carboxamide(3) |
| 94 | 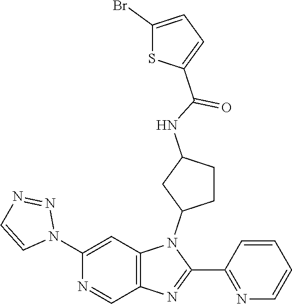 [SMILES: O=C(NC1CCC(n2c(-c3ccccn3)nc3cnc(-n4ccnn4)cc32)C1)c1ccc(Br)s1] | 5-bromo-N-(3-(2-(pyridin-2-yl)-6- (1H-1,2,3-triazol-1-yl)-1H- imidazo[4,5-c]pyridin-1- yl)cyclopentyl)thiophene-2- carboxamide(3) |
| 95 | 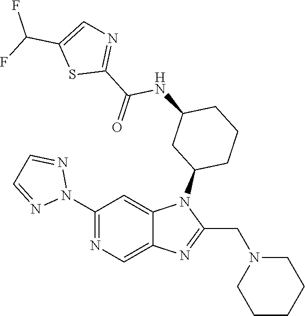 [SMILES: O=C(N[C@H]1CCC[C@@H](n2c(CN3CCCCC3)nc3cnc(-n4nccn4)cc32)C1)c1ncc(C(F)F)s1] | 5-(difluoromethyl)-N-((1S,3R)-3-(2- (piperidin-1-ylmethyl)-6-(2H-1,2,3- triazol-2-yl)-1H-imidazo[4,5- c]pyridin-1-yl)cyclohexyl)thiazole-2- carboxamide |
| 96 | 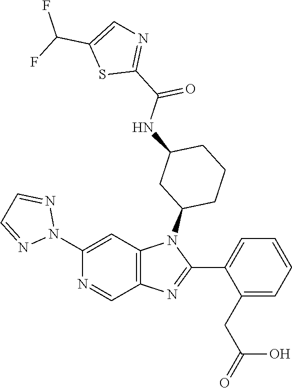 [SMILES: O=C(O)Cc1ccccc1-c1nc2cnc(-n3nccn3)cc2n1[C@@H]1CCC[C@H](NC(=O)c2ncc(C(F)F)s2)C1] | 2-(2-(1-((1R,3S)-3-(5- (difluoromethyl)thiazole-2- carboxamido)cyclohexyl)-6-(2H- 1,2,3-triazol-2-yl)-1H-imidazo[4,5- c]pyridin-2-yl)phenyl)acetic acid |
| 97 | 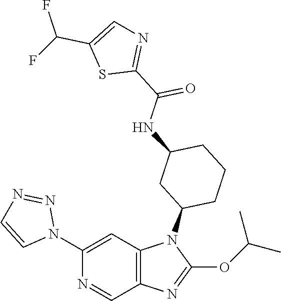 [SMILES: CC(C)Oc1nc2cnc(-n3ccnn3)cc2n1[C@@H]1CCC[C@H](NC(=O)c2ncc(C(F)F)s2)C1] | 5-(difluoromethyl)-N-((1S,3R)-3-(2- isopropoxy-6-(1H-1,2,3-triazol-1-yl)- 1H-imidazo[4,5-c]pyridin-1- yl)cyclohexyl)thiazole-2-carboxamide |
| 98 | 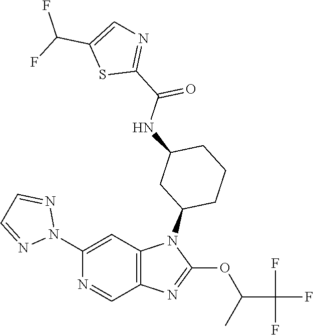 [SMILES: CC(Oc1nc2cnc(-n3nccn3)cc2n1[C@@H]1CCC[C@H](NC(=O)c2ncc(C(F)F)s2)C1)C(F)(F)F] | N-((1S,3R)-3-(6-(2H-1,2,3-triazol-2- yl)-2-((1,1,1-trifluoropropan-2- yl)oxy)-1H-imidazo[4,5-c]pyridin-1- yl)cyclohexyl)-5- (difluoromethyl)thiazole-2- carboxamide |
| 99 | 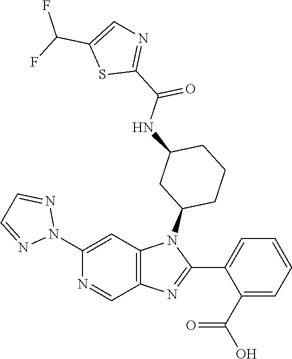 [SMILES: O=C(N[C@H]1CCC[C@@H](n2c(-c3ccccc3C(=O)O)nc3cnc(-n4nccn4)cc32)C1)c1ncc(C(F)F)s1] | 2-(1-((1R,3S)-3-(5- (difluoromethyl)thiazole-2- carboxamido)cyclohexyl)-6-(2H- 1,2,3-triazol-2-yl)-1H-imidazo[4,5- c]pyridin-2-yl)benzoic acid |
| 100 | 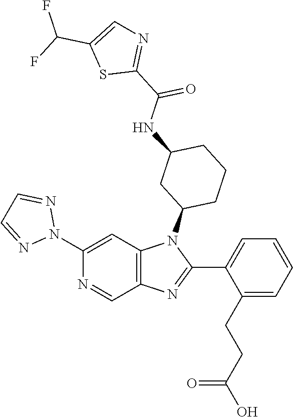 [SMILES: O=C(O)CCc1ccccc1-c1nc2cnc(-n3nccn3)cc2n1[C@@H]1CCC[C@H](NC(=O)c2ncc(C(F)F)s2)C1] | 3-(2-(1-((1R,3S)-3-(5- (difluoromethyl)thiazole-2- carboxamido)cyclohexyl)-6-(2H- 1,2,3-triazol-2-yl)-1H-imidazo[4,5- c]pyridin-2-yl)phenyl)propanoic acid |
| 101 | 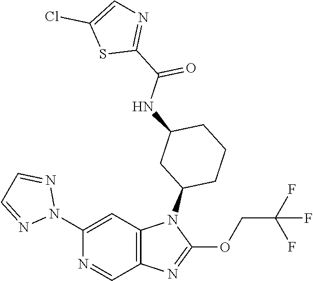 [SMILES: O=C(N[C@H]1CCC[C@@H](n2c(OCC(F)(F)F)nc3cnc(-n4nccn4)cc32)C1)c1ncc(Cl)s1] | N-((1S,3R)-3-(6-(2H-1,2,3-triazol-2- yl)-2-(2,2,2-trifluoroethoxy)-1H- imidazo[4,5-c]pyridin-1- yl)cyclohexyl)-5-chlorothiazole-2- carboxamide |
| 102 | 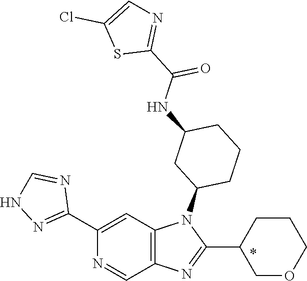 [SMILES: O=C(N[C@H]1CCC[C@@H](n2c(C3CCCOC3)nc3cnc(-c4nc[nH]n4)cc32)C1)c1ncc(Cl)s1] | 5-chloro-N-((1S,3R)-3-(2- (tetrahydro-2H-pyran-3-yl)-6-(1H- 1,2,4-triazol-3-yl)-1H-imidazo[4,5- c]pyridin-1-yl)cyclohexyl)thiazole-2- carboxamide (Diastereomer 1) |
| 103 | 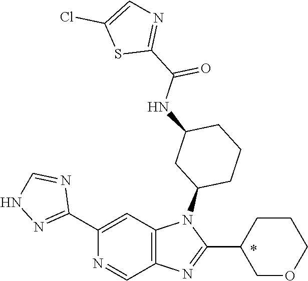 [SMILES: O=C(N[C@H]1CCC[C@@H](n2c(C3CCCOC3)nc3cnc(-c4nc[nH]n4)cc32)C1)c1ncc(Cl)s1] | 5-chloro-N-((1S,3R)-3-(2- (tetrahydro-2H-pyran-3-yl)-6-(1H- 1,2,4-triazol-3-yl)-1H-imidazo[4,5- c]pyridin-1-yl)cyclohexyl)thiazole-2- carboxamide (Diastereomer 2) |
| 104 | 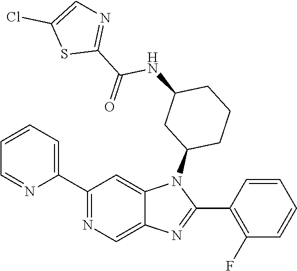 [SMILES: O=C(N[C@H]1CCC[C@@H](n2c(-c3ccccc3F)nc3cnc(-c4ccccn4)cc32)C1)c1ncc(Cl)s1] | 5-chloro-N-((1S,3R)-3-(2-(2- fluorophenyl)-6-(pyridin-2-yl)-1H- imidazo[4,5-c]pyridin-1- yl)cyclohexyl)thiazole-2-carboxamide |
| 105 | 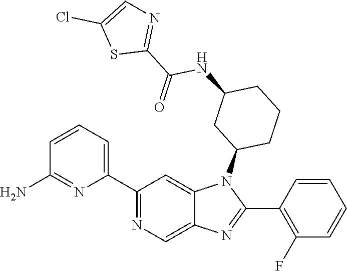 [SMILES: Nc1cccc(-c2cc3c(cn2)nc(-c2ccccc2F)n3[C@@H]2CCC[C@H](NC(=O)c3ncc(Cl)s3)C2)n1] | N-((1S,3R)-3-(6-(6-aminopyridin-2- yl)-2-(2-fluorophenyl)-1H- imidazo[4,5-c]pyridin-1- yl)cyclohexyl)-5-chlorothiazole-2- carboxamide |
| 106 | 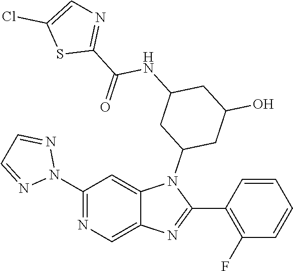 [SMILES: O=C(NC1CC(O)CC(n2c(-c3ccccc3F)nc3cnc(-n4nccn4)cc32)C1)c1ncc(Cl)s1] | 5-(difluoromethyl)-N-(3-(2-(2- fluorophenyl)-6-(2H-1,2,3-triazol-2- yl)-1H-imidazo[4,5-c]pyridin-1-yl)-5- hydroxycyclohexyl)thiazole-2- carboxamide (Mixture of Examples 107 and 108) |
| 107 | 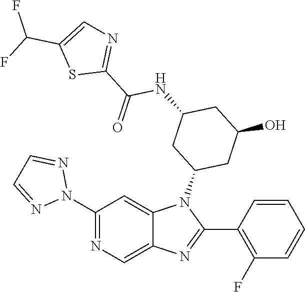 [SMILES: O=C(N[C@@H]1C[C@@H](O)C[C@H](n2c(-c3ccccc3F)nc3cnc(-n4nccn4)cc32)C1)c1ncc(C(F)F)s1] | 5-(difluoromethyl)-N-((1S,3R,5R)-3- (2-(2-fluorophenyl)-6-(2H-1,2,3- triazol-2-yl)-1H-imidazo[4,5- c]pyridin-1-yl)-5- hydroxycyclohexyl)thiazole-2- carboxamide (Enantiomer 1) |
| 108 | 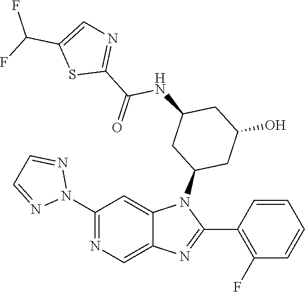 [SMILES: O=C(N[C@H]1C[C@H](O)C[C@@H](n2c(-c3ccccc3F)nc3cnc(-n4nccn4)cc32)C1)c1ncc(C(F)F)s1] | 5-(difluoromethyl)-N-(1R,3S,5S)-3- (2-(2-fluorophenyl)-6-(2H-1,2,3- triazol-2-yl)-1H-imidazo[4,5- c]pyridin-1-yl)-5- hydroxycyclohexyl)thiazole-2- carboxamide (Enantiomer 2) |
| 109 | 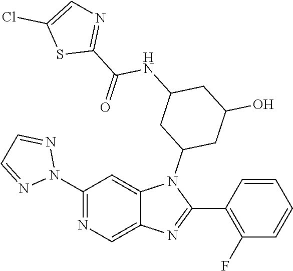 [SMILES: O=C(NC1CC(O)CC(n2c(-c3ccccc3F)nc3cnc(-n4nccn4)cc32)C1)c1ncc(Cl)s1] | 5-chloro-N-(3-(2-(2-fluorophenyl)-6- (2H-1,2,3-triazol-2-yl)-1H- imidazo[4,5-c]pyridin-1-yl)-5- hydroxycyclohexyl)thiazole-2- carboxamide (Mixture of Examples 110 and 111) |
| 110 | 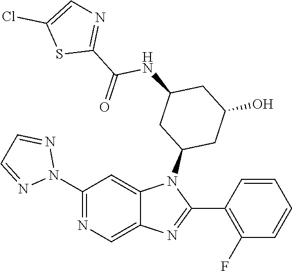 [SMILES: O=C(N[C@H]1C[C@H](O)C[C@@H](n2c(-c3ccccc3F)nc3cnc(-n4nccn4)cc32)C1)c1ncc(Cl)s1] | 5-chloro-N-((1R,3S,5S)-3-(2-(2- fluorophenyl)-6-(2H-1,2,3-triazol-2- yl)-1H-imidazo[4,5-c]pyridin-1-yl)-5- hydroxycyclohexyl)thiazole-2- carboxamide (Enantiomer 2) |
| 111 | 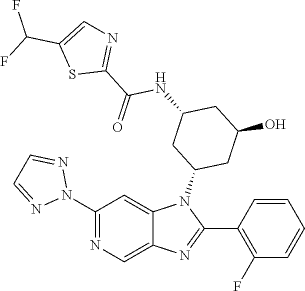 [SMILES: O=C(N[C@@H]1C[C@@H](O)C[C@H](n2c(-c3ccccc3F)nc3cnc(-n4nccn4)cc32)C1)c1ncc(C(F)F)s1] | 5-chloro-N-((1S,3R,5R)-3-(2-(2- fluorophenyl)-6-(2H-1,2,3-triazol-2- yl)-1H-imidazo[4,5-c]pyridin-1-yl)-5- hydroxycyclohexyl)thiazole-2- carboxamide (Enantiomer 1) |
| 112 | 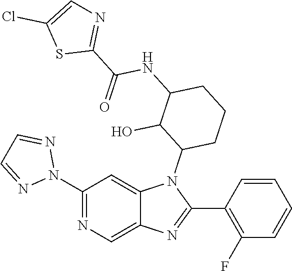 [SMILES: O=C(NC1CCCC(n2c(-c3ccccc3F)nc3cnc(-n4nccn4)cc32)C1O)c1ncc(Cl)s1] | 5-chloro-N-(3-(2-(2-fluorophenyl)-6- (2H-1,2,3-triazol-2-yl)-1H- imidazo[4,5-c]pyridin-1-yl)-2- hydroxycyclohexyl)thiazole-2- carboxamide (Mixture of Examples 113 and 114) |
| 113 | 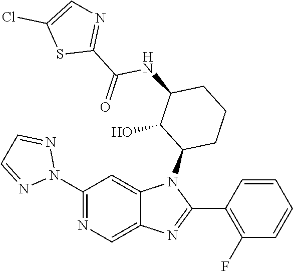 [SMILES: O=C(N[C@H]1CCC[C@@H](n2c(-c3ccccc3F)nc3cnc(-n4nccn4)cc32)[C@@H]1O)c1ncc(Cl)s1] | 5-chloro-N-((1S,2R,3R)-3-(2-(2- fluorophenyl)-6-(2H-1,2,3-triazol-2- yl)-1H-imidazo[4,5-c]pyridin-1-yl)-2- hydroxycyclohexyl)thiazole-2- carboxamide (Enantiomer 2) |
| 114 | 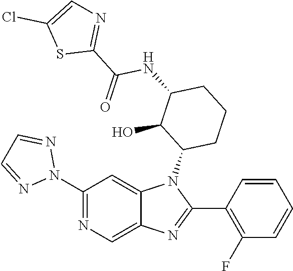 [SMILES: O=C(N[C@@H]1CCC[C@H](n2c(-c3ccccc3F)nc3cnc(-n4nccn4)cc32)[C@H]1O)c1ncc(Cl)s1] | 5-chloro-N-((1R,2S,3S)-3-(2-(2- fluorophenyl)-6-(2H-1,2,3-triazol-2- yl)-1H-imidazo[4,5-c]pyridin-1-yl)-2- hydroxycyclohexyl)thiazole-2- carboxamide (Enantiomer 1) |
| 115 | 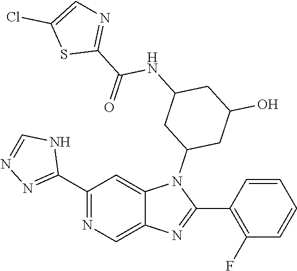 [SMILES: O=C(NC1CC(O)CC(n2c(-c3ccccc3F)nc3cnc(-c4nnc[nH]4)cc32)C1)c1ncc(Cl)s1] | 5-chloro-N-(3-(2-(2-fluorophenyl)-6- (4H-1,2,4-triazol-3-yl)-1H- imidazo[4,5-c]pyridin-1-yl)-5- hydroxycyclohexyl)thiazole-2- carboxamide(7) |
| 116 | 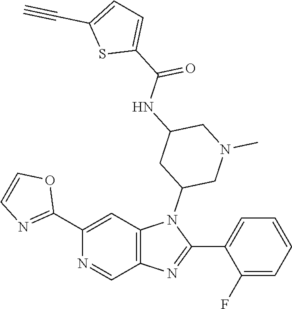 [SMILES: C#Cc1ccc(C(=O)NC2CC(n3c(-c4ccccc4F)nc4cnc(-c5ncco5)cc43)CN(C)C2)s1] | 5-ethynyl-N-(5-(2-(2-fluorophenyl)- 6-(oxazol-2-yl)-1H-imidazo[4,5- c]pyridin-1-yl)-1-methylpiperidin-3- yl)thiophene-2-carboxamide(4) |
| 117 | 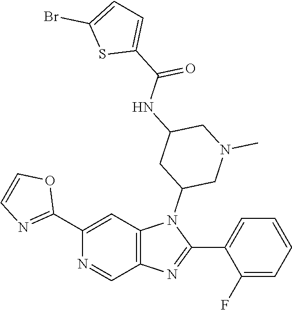 [SMILES: CN1CC(NC(=O)c2ccc(Br)s2)CC(n2c(-c3ccccc3F)nc3cnc(-c4ncco4)cc32)C1] | 5-bromo-N-(5-(2-(2-fluorophenyl)-6- (oxazol-2-yl)-1H-imidazo[4,5- c]pyridin-1-yl)-1-methylpiperidin-3- yl)thiophene-2-carboxamide(4) |
| 118 | 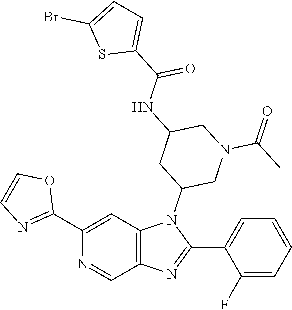 [SMILES: CC(=O)N1CC(NC(=O)c2ccc(Br)s2)CC(n2c(-c3ccccc3F)nc3cnc(-c4ncco4)cc32)C1] | N-(1-acetyl-5-(2-(2-fluorophenyl)-6- (oxazol-2-yl)-1H-imidazo[4,5- c]pyridin-1-yl)piperidin-3-yl)-5- bromothiophene-2-carboxamide(4) |
| 119 | 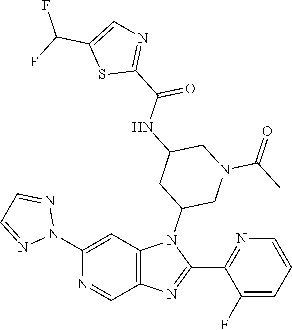 [SMILES: CC(=O)N1CC(NC(=O)c2ncc(C(F)F)s2)CC(n2c(-c3ncccc3F)nc3cnc(-n4nccn4)cc32)C1] | N-(1-acetyl-5-(2-(3-fluoropyridin-2- yl)-6-(2H-1,2,3-triazol-2-yl)-1H- imidazo[4,5-c]pyridin-1-yl)piperidin- 3-yl)-5-(difluoromethyl)thiazole-2- carboxamide(4) |
| 120 | 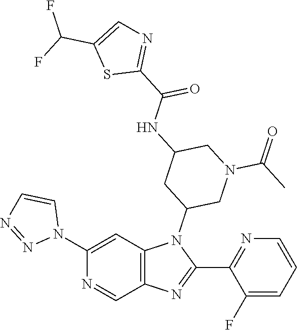 [SMILES: CC(=O)N1CC(NC(=O)c2ncc(C(F)F)s2)CC(n2c(-c3ncccc3F)nc3cnc(-n4ccnn4)cc32)C1] | N-(1-acetyl-5-(2-(3-fluoropyridin-2- yl)-6-(1H-1,2,3-triazol-1-yl)-1H- imidazo[4,5-c]pyridin-1-yl)piperidin- 3-yl)-5-(difluoromethyl)thiazole-2- carboxamide(4) |
| 121 | 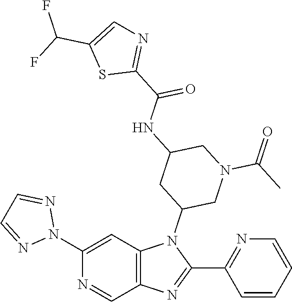 [SMILES: CC(=O)N1CC(NC(=O)c2ncc(C(F)F)s2)CC(n2c(-c3ccccn3)nc3cnc(-n4nccn4)cc32)C1] | N-(1-acetyl-5-(2-(pyridin-2-yl)-6- (2H-1,2,3-triazol-2-yl)-1H- imidazo[4,5-c]pyridin-1-yl)piperidin- 3-yl)-5-(difluoromethyl)thiazole-2- carboxamide(4) |
| 122 | 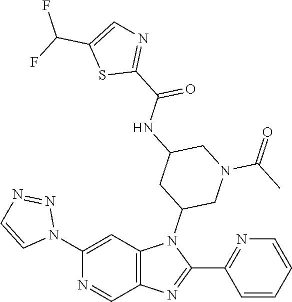 [SMILES: CC(=O)N1CC(NC(=O)c2ncc(C(F)F)s2)CC(n2c(-c3ccccn3)nc3cnc(-n4ccnn4)cc32)C1] | N-(1-acetyl-5-(2-(pyridin-2-yl)-6- (1H-1,2,3-triazol-1-yl)-1H- imidazo[4,5-c]pyridin-1-yl)piperidin- 3-yl)-5-(difluoromethyl)thiazole-2- carboxamide(4) |
| 123 | 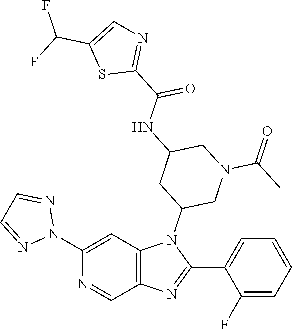 [SMILES: CC(=O)N1CC(NC(=O)c2ncc(C(F)F)s2)CC(n2c(-c3ccccc3F)nc3cnc(-n4nccn4)cc32)C1] | N-(1-acetyl-5-(2-(2-fluorophenyl)-6- (2H-1,2,3-triazol-2-yl)-1H- imidazo[4,5-c]pyridin-1-yl)piperidin- 3-yl)-5-(difluoromethyl)thiazole-2- carboxamide(4) |
| 124 | 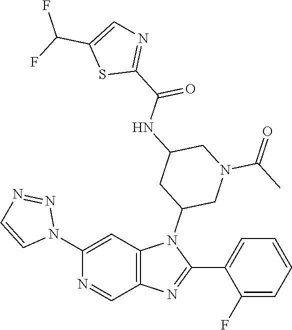 [SMILES: CC(=O)N1CC(NC(=O)c2ncc(C(F)F)s2)CC(n2c(-c3ccccc3F)nc3cnc(-n4ccnn4)cc32)C1] | N-(1-acetyl-5-(2-(2-fluorophenyl)-6- (1H-1,2,3-triazol-1-yl)-1H- imidazo[4,5-c]pyridin-1-yl)piperidin- 3-yl)-5-(difluoromethyl)thiazole-2- carboxamide(4) |
| 125 | 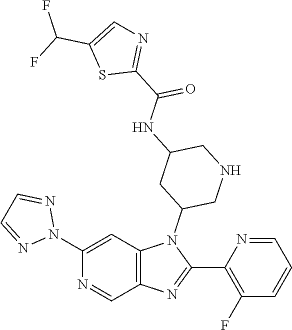 [SMILES: O=C(NC1CNCC(n2c(-c3ncccc3F)nc3cnc(-n4nccn4)cc32)C1)c1ncc(C(F)F)s1] | 5-(difluoromethyl)-N-(5-(2-(3- fluoropyridin-2-yl)-6-(2H-1,2,3- triazol-2-yl)-1H-imidazo[4,5- c]pyridin-1-yl)piperidin-3-yl)thiazole- 2-carboxamide(4) |
| 126 | 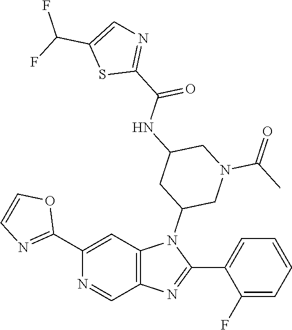 [SMILES: CC(=O)N1CC(NC(=O)c2ncc(C(F)F)s2)CC(n2c(-c3ccccc3F)nc3cnc(-c4ncco4)cc32)C1] | N-(1-acetyl-5-(2-(2-fluorophenyl)-6- (oxazol-2-yl)-1H-imidazo[4,5- c]pyridin-1-yl)piperidin-3-yl)-5- (difluoromethyl)thiazole-2- carboxamide(4) |
| 127 | 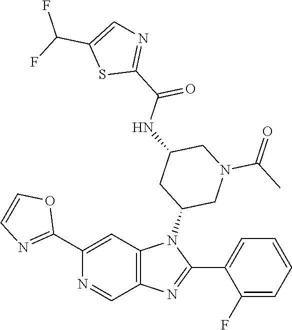 [SMILES: CC(=O)N1C[C@@H](NC(=O)c2ncc(C(F)F)s2)C[C@@H](n2c(-c3ccccc3F)nc3cnc(-c4ncco4)cc32)C1] | N-((3S,5R)-1-acetyl-5-(2-(2- fluorophenyl)-6-(oxazol-2-yl)-1H- imidazo[4,5-c]pyridin-1-yl)piperidin- 3-yl)-5-(difluoromethyl)thiazole-2- carboxamide (Enantiomer 1) |
| 128 | 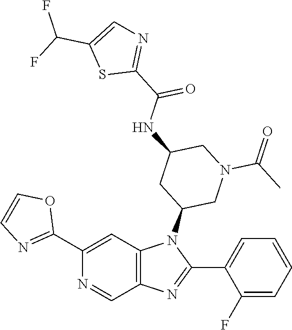 [SMILES: CC(=O)N1C[C@H](NC(=O)c2ncc(C(F)F)s2)C[C@H](n2c(-c3ccccc3F)nc3cnc(-c4ncco4)cc32)C1] | N-((3R,5S)-1-acetyl-5-(2-(2- fluorophenyl)-6-(oxazol-2-yl)-1H- imidazo[4,5-c]pyridin-1-yl)piperidin- 3-yl)-5-(difluoromethyl)thiazole-2- carboxamide (Enantiomer 2) |
| 129 | 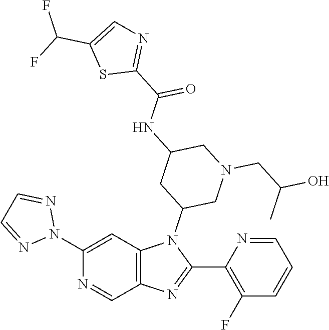 [SMILES: CC(O)CN1CC(NC(=O)c2ncc(C(F)F)s2)CC(n2c(-c3ncccc3F)nc3cnc(-n4nccn4)cc32)C1] | 5-(difluoromethyl)-N-(5-(2-(3- fluoropyridin-2-yl)-6-(2H-1,2,3- triazol-2-yl)-1H-imidazo[4,5- c]pyridin-1-yl)-1-(2- hydroxypropyl)piperidin-3- yl)thiazole-2-carboxamide(4) |
| 130 | 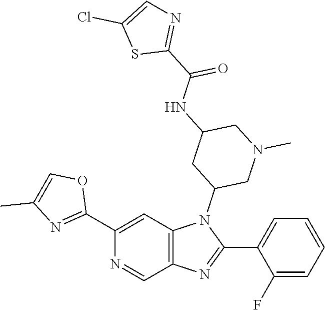 [SMILES: Cc1coc(-c2cc3c(cn2)nc(-c2ccccc2F)n3C2CC(NC(=O)c3ncc(Cl)s3)CN(C)C2)n1] | 5-chloro-N-(5-(2-(2-fluorophenyl)-6- (4-methyloxazol-2-yl)-1H- imidazo[4,5-c]pyridin-1-yl)-1- methylpiperidin-3-yl)thiazole-2- carboxamide(4) |
| 131 | 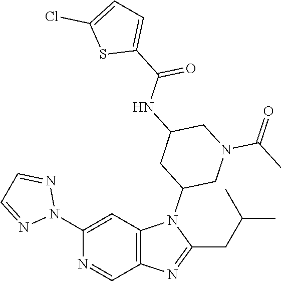 [SMILES: CC(=O)N1CC(NC(=O)c2ccc(Cl)s2)CC(n2c(CC(C)C)nc3cnc(-n4nccn4)cc32)C1] | N-(1-acetyl-5-(2-isobutyl-6-(2H- 1,2,3-triazol-2-yl)-1H-imidazo[4,5- c]pyridin-1-yl)piperidin-3-yl)-5- chlorothiophene-2-carboxamide(4) |
| 132 | 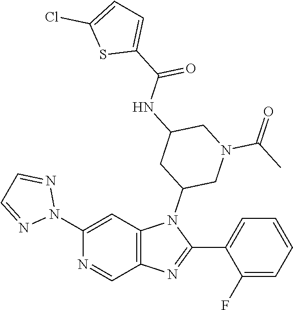 [SMILES: CC(=O)N1CC(NC(=O)c2ccc(Cl)s2)CC(n2c(-c3ccccc3F)nc3cnc(-n4nccn4)cc32)C1] | N-(1-acetyl-5-(2-(2-fluorophenyl)-6- (2H-1,2,3-triazol-2-yl)-1H- imidazo[4,5-c]pyridin-1-yl)piperidin- 3-yl)-5-chlorothiophene-2- carboxamide(4) |
| 133 | 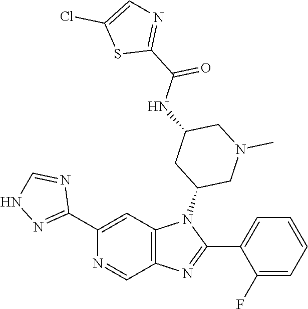 [SMILES: CN1C[C@@H](NC(=O)c2ncc(Cl)s2)C[C@@H](n2c(-c3ccccc3F)nc3cnc(-c4nc[nH]n4)cc32)C1] | 5-chloro-N-((3S,5R)-5-(2-(2- fluorophenyl)-6-(1H-1,2,4-triazol-3- yl)-1H-imidazo[4,5-c]pyridin-1-yl)-1- methylpiperidin-3-yl)thiazole-2- carboxamide (Enantiomer 1) |
| 134 | 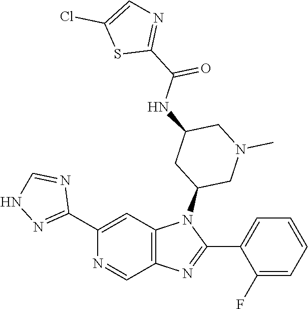 [SMILES: CN1C[C@H](NC(=O)c2ncc(Cl)s2)C[C@H](n2c(-c3ccccc3F)nc3cnc(-c4nc[nH]n4)cc32)C1] | 5-chloro-N-((3R,5S)-5-(2-(2- fluorophenyl)-6-(1H-1,2,4-triazol-3- yl)-1H-imidazo[4,5-c]pyridin-1-yl)-1- methylpiperidin-3-yl)thiazole-2- carboxamide (Enantiomer 2) |
| 135 | 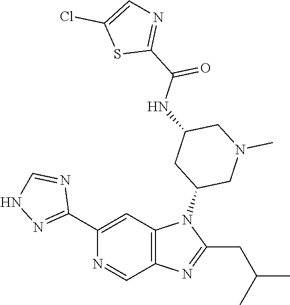 [SMILES: CC(C)Cc1nc2cnc(-c3nc[nH]n3)cc2n1[C@@H]1C[C@H](NC(=O)c2ncc(Cl)s2)CN(C)C1] | 5-chloro-N-((3S,5R)-5-(2-isobutyl-6- (1H-1,2,4-triazol-3-yl)-1H- imidazo[4,5-c]pyridin-1-yl)-1- methylpiperidin-3-yl)thiazole-2- carboxamide (Enantiomer 1) |
| 136 | 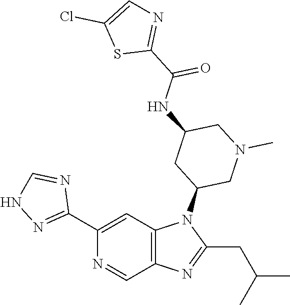 [SMILES: CC(C)Cc1nc2cnc(-c3nc[nH]n3)cc2n1[C@H]1C[C@@H](NC(=O)c2ncc(Cl)s2)CN(C)C1] | 5-chloro-N-((3R,5S)-5-(2-isobutyl-6- (1H-1,2,4-triazol-3-yl)-1H- imidazo[4,5-c]pyridin-1-yl)-1- methylpiperidin-3-yl)thiazole-2- carboxamide (Enantiomer 2) |
| 137 | 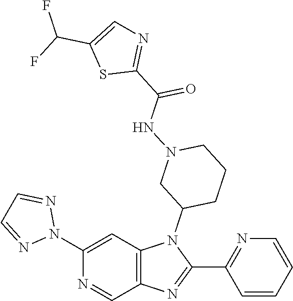 [SMILES: O=C(NN1CCCC(n2c(-c3ccccn3)nc3cnc(-n4nccn4)cc32)C1)c1ncc(C(F)F)s1] | 5-(difluoromethyl)-N-(3-(2-(pyridin- 2-yl)-6-(2H-1,2,3-triazol-2-yl)-1H- imidazo[4,5-c]pyridin-1-yl)piperidin- 1-yl)thiazole-2-carboxamide |
| 138 | 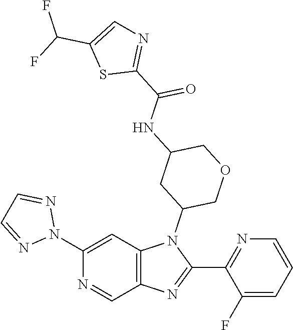 [SMILES: O=C(NC1COCC(n2c(-c3ncccc3F)nc3cnc(-n4nccn4)cc32)C1)c1ncc(C(F)F)s1] | 5-(difluoromethyl)-N-(5-(2-(3- fluoropyridin-2-yl)-6-(2H-1,2,3- triazol-2-yl)-1H-imidazo[4,5- c]pyridin-1-yl)tetrahydro-2H-pyran- 3-yl)thiazole-2-carboxamide(5) |
| 139 | 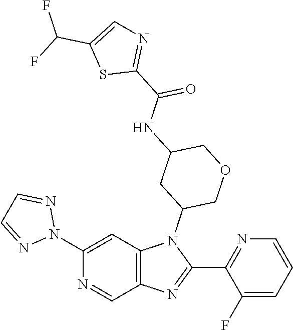 [SMILES: O=C(NC1COCC(n2c(-c3ncccc3F)nc3cnc(-n4nccn4)cc32)C1)c1ncc(C(F)F)s1] | 5-(difluoromethyl)-N-(5-(2-(3- fluoropyridin-2-yl)-6-(2H-1,2,3- triazol-2-yl)-1H-imidazo[4,5- c]pyridin-1-yl)tetrahydro-2H-pyran- 3-yl)thiazole-2-carboxamide(6) |
| 140 | 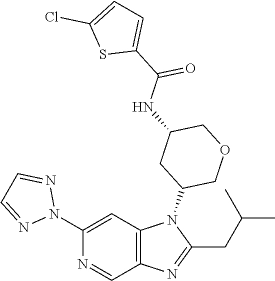 | 5-chloro-N-((3S,5R)-5-(2-isobutyl-6- (2H-1,2,3-triazol-2-yl)-1H- imidazo[4,5-c]pyridin-1- yl)tetrahydro-2H-pyran-3- yl)thiophene-2-carboxamide (Enantiomer 1) |
| 141 | 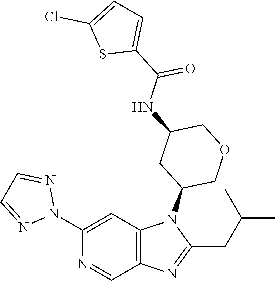 [SMILES: CC(C)Cc1nc2cnc(-n3nccn3)cc2n1[C@@H]1COC[C@H](NC(=O)c2ccc(Cl)s2)C1] | 5-chloro-N-((3R,5S)-5-(2-isobutyl-6- (2H-1,2,3-triazol-2-yl)-1H- imidazo[4,5-c]pyridin-1- yl)tetrahydro-2H-pyran-3- yl)thiophene-2-carboxamide (Enantiomer 2) |
| 142 | 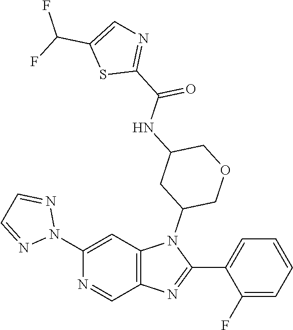 [SMILES: O=C(NC1COCC(n2c(-c3ccccc3F)nc3cnc(-n4nccn4)cc32)C1)c1ncc(C(F)F)s1] | 5-(difluoromethyl)-N-(5-(2-(2- fluorophenyl)-6-(2H-1,2,3-triazol-2- yl)-1H-imidazo[4,5-c]pyridin-1- yl)tetrahydro-2H-pyran-3-yl)thiazole- 2-carboxamide(5) |
| 143 | 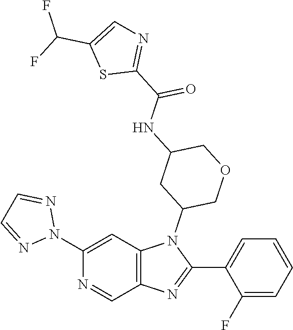 [SMILES: O=C(NC1COCC(n2c(-c3ccccc3F)nc3cnc(-n4nccn4)cc32)C1)c1ncc(C(F)F)s1] | 5-(difluoromethyl)-N-(5-(2-(2- fluorophenyl)-6-(2H-1,2,3-triazol-2- yl)-1H-imidazo[4,5-c]pyridin-1- yl)tetrahydro-2H-pyran-3-yl)thiazole- 2-carboxamide(6) |
| 144 | 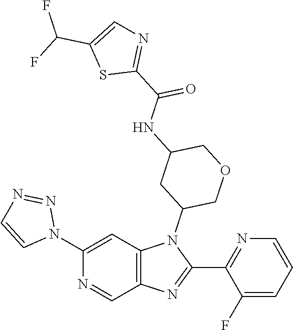 [SMILES: O=C(NC1COCC(n2c(-c3ncccc3F)nc3cnc(-n4ccnn4)cc32)C1)c1ncc(C(F)F)s1] | 5-(difluoromethyl)-N-(5-(2-(3- fluoropyridin-2-yl)-6-(1H-1,2,3- triazol-1-yl)-1H-imidazo[4,5- c]pyridin-1-yl)tetrahydro-2H-pyran- 3-yl)thiazole-2-carboxamide(5) |
| 145 | 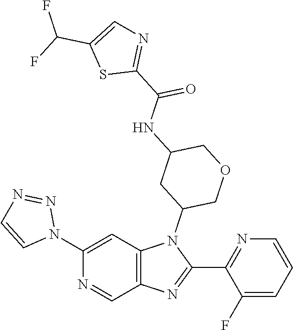 [SMILES: O=C(NC1COCC(n2c(-c3ncccc3F)nc3cnc(-n4ccnn4)cc32)C1)c1ncc(C(F)F)s1] | 5-(difluoromethyl)-N-(5-(2-(3- fluoropyridin-2-yl)-6-(1H-1,2,3- triazol-1-yl)-1H-imidazo[4,5- c]pyridin-1-yl)tetrahydro-2H-pyran- 3-yl)thiazole-2-carboxamide(6) |
| 146 | 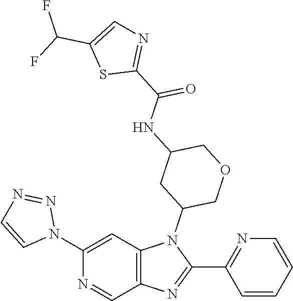 [SMILES: O=C(NC1COCC(n2c(-c3ccccn3)nc3cnc(-n4ccnn4)cc32)C1)c1ncc(C(F)F)s1] | 5-(difluoromethyl)-N-(5-(2-(pyridin- 2-yl)-6-(1H-1,2,3-triazol-1-yl)-1H- imidazo[4,5-c]pyridin-1- yl)tetrahydro-2H-pyran-3-yl)thiazole- 2-carboxamide(5) |
| 147 | 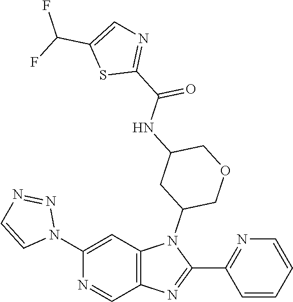 [SMILES: O=C(NC1COCC(n2c(-c3ccccn3)nc3cnc(-n4ccnn4)cc32)C1)c1ncc(C(F)F)s1] | 5-(difluoromethyl)-N-(5-(2-(pyridin- 2-yl)-6-(1H-1,2,3-triazol-1-yl)-1H- imidazo[4,5-c]pyridin-1- yl)tetrahydro-2H-pyran-3-yl)thiazole- 2-carboxamide(6) |
| 148 | 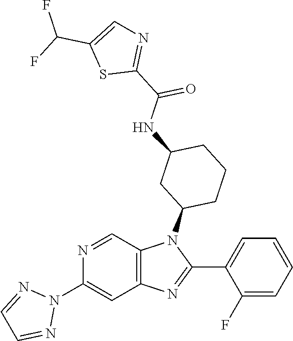 [SMILES: O=C(N[C@H]1CCC[C@@H](n2c(-c3ccccc3F)nc3cc(-n4nccn4)ncc32)C1)c1ncc(C(F)F)s1] | 5-(difluoromethyl)-N-((1S,3R)-3-(2- (2-fluorophenyl)-6-(2H-1,2,3-triazol- 2-yl)-3H-imidazo[4,5-c]pyridin-3- yl)cyclohexyl)thiazole-2-carboxamide |
| 149 | 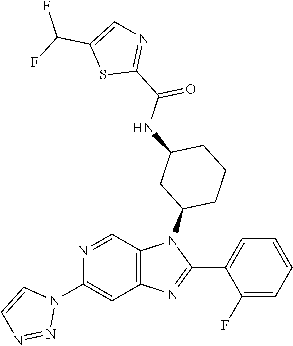 [SMILES: O=C(N[C@H]1CCC[C@@H](n2c(-c3ccccc3F)nc3cc(-n4ccnn4)ncc32)C1)c1ncc(C(F)F)s1] | 5-(difluoromethyl)-N-((1S,3R)-3-(2- (2-fluorophenyl)-6-(1H-1,2,3-triazol- 1-yl)-3H-imidazo[4,5-c]pyridin-3- yl)cyclohexyl)thiazole-2-carboxamide |
| 150 | 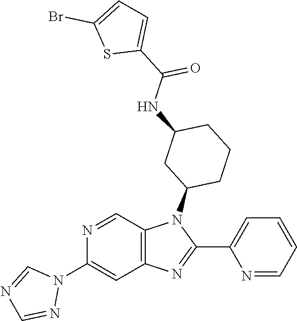 [SMILES: O=C(N[C@H]1CCC[C@@H](n2c(-c3ccccn3)nc3cc(-n4cncn4)ncc32)C1)c1ccc(Br)s1] | 5-bromo-N-((1S,3R)-3-(2-(pyridin-2- yl)-6-(1H-1,2,4-triazol-1-yl)-3H- imidazo[4,5-c]pyridin-3- yl)cyclohexyl)thiophene-2- carboxamide |
| 151 | 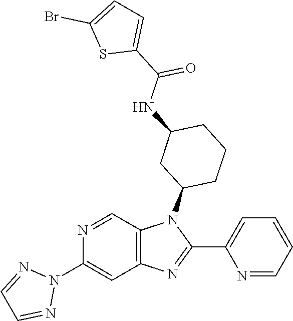 [SMILES: O=C(N[C@H]1CCC[C@@H](n2c(-c3ccccn3)nc3cc(-n4nccn4)ncc32)C1)c1ccc(Br)s1] | 5-bromo-N-((1S,3R)-3-(2-(pyridin-2- yl)-6-(2H-1,2,3-triazol-2-yl)-3H- imidazo[4,5-c]pyridin-3- yl)cyclohexyl)thiophene-2- carboxamide |
| 152 | 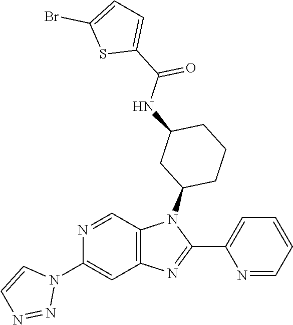 [SMILES: O=C(N[C@H]1CCC[C@@H](n2c(-c3ccccn3)nc3cc(-n4ccnn4)ncc32)C1)c1ccc(Br)s1] | 5-bromo-N-((1S,3R)-3-(2-(pyridin-2- yl)-6-(1H-1,2,3-triazol-1-yl)-3H- imidazo[4,5-c]pyridin-3- yl)cyclohexyl)thiophene-2- carboxamide |
| 153 | 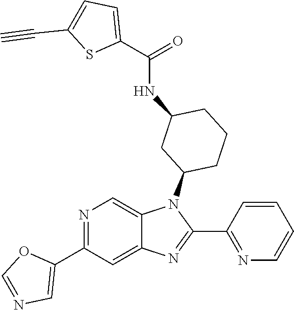 [SMILES: C#Cc1ccc(C(=O)N[C@H]2CCC[C@@H](n3c(-c4ccccn4)nc4cc(-c5cnco5)ncc43)C2)s1] | 5-ethynyl-N-((1S,3R)-3-(6-(oxazol-5- yl)-2-(pyridin-2-yl)-3H-imidazo[4,5- c]pyridin-3-yl)cyclohexyl)thiophene- 2-carboxamide |
| 154 | 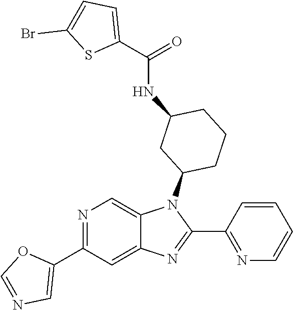 [SMILES: O=C(N[C@H]1CCC[C@@H](n2c(-c3ccccn3)nc3cc(-c4cnco4)ncc32)C1)c1ccc(Br)s1] | 5-bromo-N-((1S,3R)-3-(6-(oxazol-5- yl)-2-(pyridin-2-yl)-3H-imidazo[4,5- c]pyridin-3-yl)cyclohexyl)thiophene- 2-carboxamide |
| 155 | 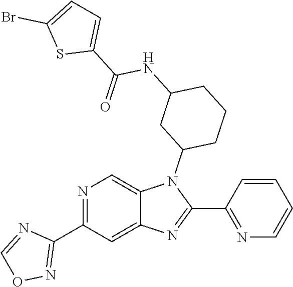 [SMILES: O=C(NC1CCCC(n2c(-c3ccccn3)nc3cc(-c4ncon4)ncc32)C1)c1ccc(Br)s1] | N-(3-(6-(1,2,4-oxadiazol-3-yl)-2- (pyridin-2-yl)-3H-imidazo[4,5- c]pyridin-3-yl)cyclohexyl)-5- bromothiophene-2-carboxamide(1) |
| 156 | 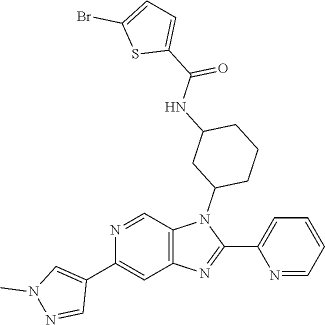 [SMILES: Cn1cc(-c2cc3nc(-c4ccccn4)n(C4CCCC(NC(=O)c5ccc(Br)s5)C4)c3cn2)cn1] | 5-bromo-N-(3-(6-(1-methyl-1H- pyrazol-4-yl)-2-(pyridin-2-yl)-3H- imidazo[4,5-c]pyridin-3- yl)cyclohexyl)thiophene-2- carboxamide(1) |
| 157 | 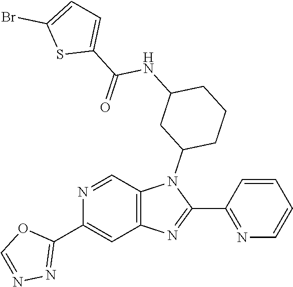 [SMILES: O=C(NC1CCCC(n2c(-c3ccccn3)nc3cc(-c4nnco4)ncc32)C1)c1ccc(Br)s1] | N-(3-(6-(1,3,4-oxadiazol-2-yl)-2- (pyridin-2-yl)-3H-imidazo[4,5- c]pyridin-3-yl)cyclohexyl)-5- bromothiophene-2-carboxamide(1) |
| 158 | 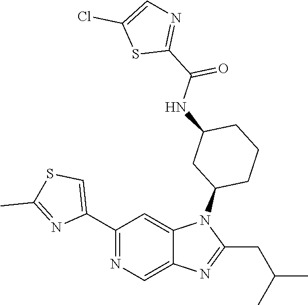 [SMILES: Cc1nc(-c2cc3c(cn2)nc(CC(C)C)n3[C@@H]2CCC[C@H](NC(=O)c3ncc(Cl)s3)C2)cs1] | 5-chloro-N-((1S,3R)-3-(2-isobutyl-6- (2-methylthiazol-4-yl)-1H- imidazo[4,5-c]pyridin-1- yl)cyclohexyl)thiazole-2-carboxamide |
| 159 | 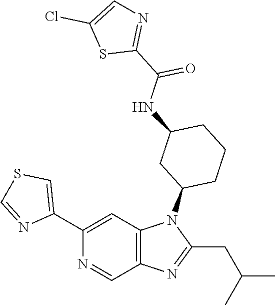 [SMILES: CC(C)Cc1nc2cnc(-c3cscn3)cc2n1[C@@H]1CCC[C@H](NC(=O)c2ncc(Cl)s2)C1] | 5-chloro-N-((1S,3R)-3-(2-isobutyl-6- (thiazol-4-yl)-1H-imidazo[4,5- c]pyridin-1-yl)cyclohexyl)thiazole-2- carboxamide |
| 160 | 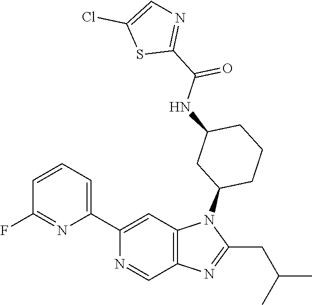 [SMILES: CC(C)Cc1nc2cnc(-c3cccc(F)n3)cc2n1[C@@H]1CCC[C@H](NC(=O)c2ncc(Cl)s2)C1] | 5-chloro-N-((1S,3R)-3-(6-(6- fluoropyridin-2-yl)-2-isobutyl-1H- imidazo[4,5-c]pyridin-1- yl)cyclohexyl)thiazole-2-carboxamide |
| 161 | 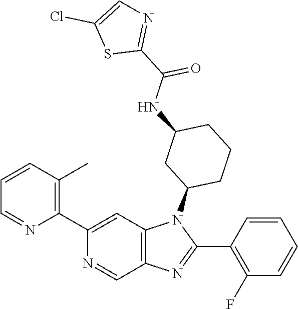 [SMILES: Cc1cccnc1-c1cc2c(cn1)nc(-c1ccccc1F)n2[C@@H]1CCC[C@H](NC(=O)c2ncc(Cl)s2)C1] | 5-chloro-N-((1S,3R)-3-(2-(2- fluorophenyl)-6-(3-methylpyridin-2- yl)-1H-imidazo[4,5-c]pyridin-1- yl)cyclohexyl)thiazole-2-carboxamide |
| 162 | 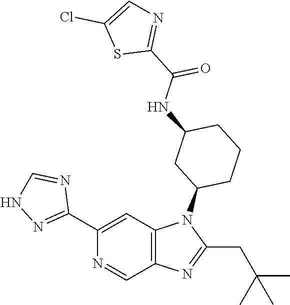 [SMILES: CC(C)(C)Cc1nc2cnc(-c3nc[nH]n3)cc2n1[C@@H]1CCC[C@H](NC(=O)c2ncc(Cl)s2)C1] | 5-chloro-N-((1S,3R)-3-(2-neopentyl- 6-(1H-1,2,4-triazol-3-yl)-1H- imidazo[4,5-c]pyridin-1- yl)cyclohexyl)thiazole-2-carboxamide |
| 163 | 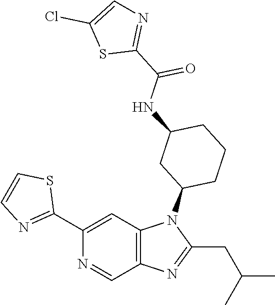 [SMILES: CC(C)Cc1nc2cnc(-c3nccs3)cc2n1[C@@H]1CCC[C@H](NC(=O)c2ncc(Cl)s2)C1] | 5-chloro-N-((1S,3R)-3-(2-isobutyl-6- (thiazol-2-yl)-1H-imidazo[4,5- c]pyridin-1-yl)cyclohexyl)thiazole-2- carboxamide |
| 164 | 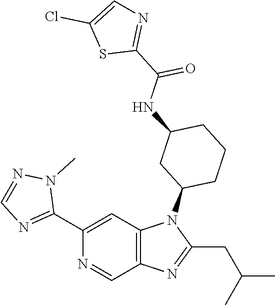 [SMILES: CC(C)Cc1nc2cnc(-c3ncnn3C)cc2n1[C@@H]1CCC[C@H](NC(=O)c2ncc(Cl)s2)C1] | 5-chloro-N-((1S,3R)-3-(2-isobutyl-6- (1-methyl-1H-1,2,4-triazol-5-yl)-1H- imidazo[4,5-c]pyridin-1- yl)cyclohexyl)thiazole-2-carboxamide |
| 165 | 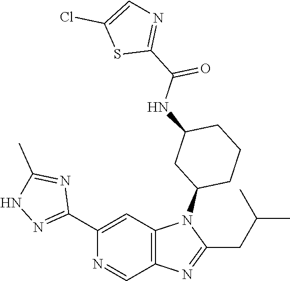 [SMILES: Cc1nc(-c2cc3c(cn2)nc(CC(C)C)n3[C@@H]2CCC[C@H](NC(=O)c3ncc(Cl)s3)C2)n[nH]1] | 5-chloro-N-((1S,3R)-3-(2-isobutyl-6- (5-methyl-1H-1,2,4-triazol-3-yl)-1H- imidazo[4,5-c]pyridin-1- yl)cyclohexyl)thiazole-2-carboxamide |
| 166 | 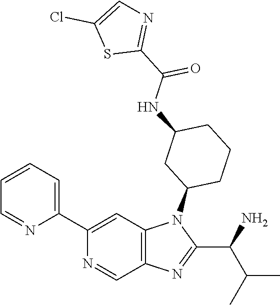 [SMILES: CC(C)[C@H](N)c1nc2cnc(-c3ccccn3)cc2n1[C@@H]1CCC[C@H](NC(=O)c2ncc(Cl)s2)C1] | N-((1S,3R)-3-(2-((S)-1-amino-2- methylpropyl)-6-(pyridin-2-yl)-1H- imidazo[4,5-c]pyridin-1- yl)cyclohexyl)-5-chlorothiazole-2- carboxamide |
| 167 | 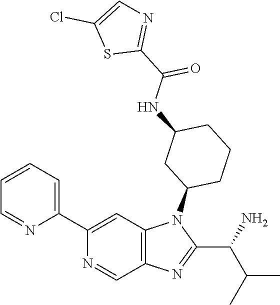 [SMILES: CC(C)[C@@H](N)c1nc2cnc(-c3ccccn3)cc2n1[C@@H]1CCC[C@H](NC(=O)c2ncc(Cl)s2)C1] | N-((1S,3R)-3-(2-((R)-1-amino-2- methylpropyl)-6-(pyridin-2-yl)-1H- imidazo[4,5-c]pyridin-1- yl)cyclohexyl)-5-chlorothiazole-2- carboxamide |
| 168 | 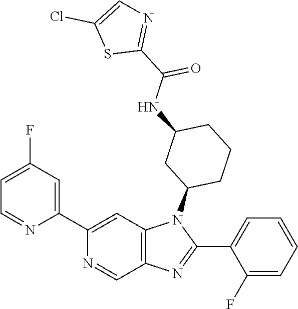 [SMILES: O=C(N[C@H]1CCC[C@@H](n2c(-c3ccccc3F)nc3cnc(-c4cc(F)ccn4)cc32)C1)c1ncc(Cl)s1] | 5-chloro-N-((1S,3R)-3-(2-(2- fluorophenyl)-6-(4-fluoropyridin-2- yl)-1H-imidazo[4,5-c]pyridin-1- yl)cyclohexyl)thiazole-2-carboxamide |
| 169 | 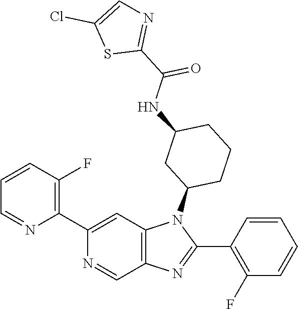 [SMILES: O=C(N[C@H]1CCC[C@@H](n2c(-c3ccccc3F)nc3cnc(-c4ncccc4F)cc32)C1)c1ncc(Cl)s1] | 5-chloro-N-((1S,3R)-3-(2-(2- fluorophenyl)-6-(3-fluoropyridin-2- yl)-1H-imidazo[4,5-c]pyridin-1- yl)cyclohexyl)thiazole-2-carboxamide |
| 170 | 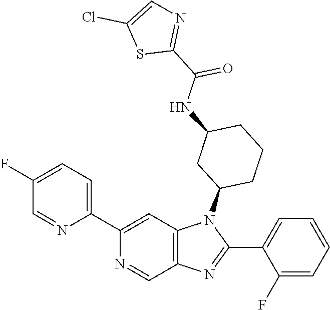 [SMILES: O=C(N[C@H]1CCC[C@@H](n2c(-c3ccccc3F)nc3cnc(-c4ccc(F)cn4)cc32)C1)c1ncc(Cl)s1] | 5-chloro-N-((1S,3R)-3-(2-(2- fluorophenyl)-6-(5-fluoropyridin-2- yl)-1H-imidazo[4,5-c]pyridin-1- yl)cyclohexyl)thiazole-2-carboxamide |
| 171 | 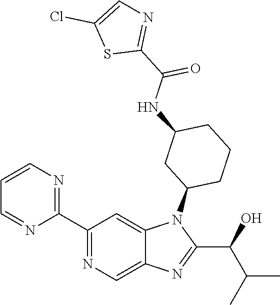 [SMILES: CC(C)[C@H](O)c1nc2cnc(-c3ncccn3)cc2n1[C@@H]1CCC[C@H](NC(=O)c2ncc(Cl)s2)C1] | 5-chloro-N-((1S,3R)-3-(2-((S)-1- hydroxy-2-methylpropyl)-6- (pyrimidin-2-yl)-1H-imidazo[4,5- c]pyridin-1-yl)cyclohexyl)thiazole-2- carboxamide |
| 172 | 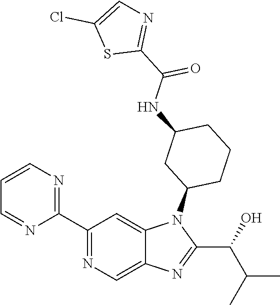 [SMILES: CC(C)[C@@H](O)c1nc2cnc(-c3ncccn3)cc2n1[C@@H]1CCC[C@H](NC(=O)c2ncc(Cl)s2)C1] | 5-chloro-N-((1S,3R)-3-(2-((R)-1- hydroxy-2-methylpropyl)-6- (pyrimidin-2-yl)-1H-imidazo[4,5- c]pyridin-1-yl)cyclohexyl)thiazole-2- carboxamide |
| 173 | 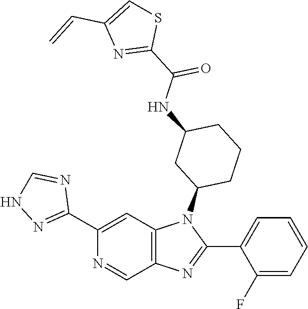 [SMILES: C=Cc1csc(C(=O)N[C@H]2CCC[C@@H](n3c(-c4ccccc4F)nc4cnc(-c5nc[nH]n5)cc43)C2)n1] | N-((1S,3R)-3-(2-(2-fluorophenyl)-6- (1H-1,2,4-triazol-3-yl)-1H- imidazo[4,5-c]pyridin-1- yl)cyclohexyl)-4-vinylthiazole-2- carboxamide |
| 174 | 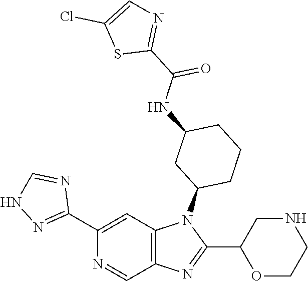 [SMILES: O=C(N[C@H]1CCC[C@@H](n2c(C3CNCCO3)nc3cnc(-c4nc[nH]n4)cc32)C1)c1ncc(Cl)s1] | 5-chloro-N-((1S,3R)-3-(2- (morpholin-2-yl)-6-(1H-1,2,4-triazol- 3-yl)-1H-imidazo[4,5-c]pyridin-1- yl)cyclohexyl)thiazole-2-carboxamide |
| 175 | 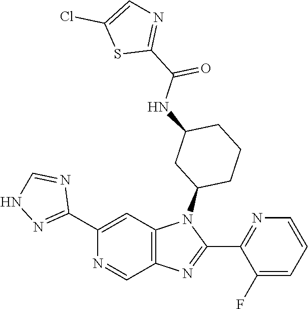 [SMILES: O=C(N[C@H]1CCC[C@@H](n2c(-c3ncccc3F)nc3cnc(-c4nc[nH]n4)cc32)C1)c1ncc(Cl)s1] | 5-chloro-N-((1S,3R)-3-(2-(3- fluoropyridin-2-yl)-6-(1H-1,2,4- triazol-3-yl)-1H-imidazo[4,5- c]pyridin-1-yl)cyclohexyl)thiazole-2- carboxamide |
| 176 | 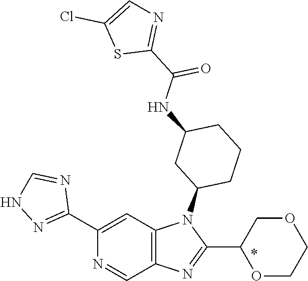 [SMILES: O=C(N[C@H]1CCC[C@@H](n2c(C3COCCO3)nc3cnc(-c4nc[nH]n4)cc32)C1)c1ncc(Cl)s1] | N-((1S,3R)-3-(2-(1,4-dioxan-2-yl)-6- (1H-1,2,4-triazol-3-yl)-1H- imidazo[4,5-c]pyridin-1- yl)cyclohexyl)-5-chlorothiazole-2- carboxamide (Diastereomer #2) |
| 177 | 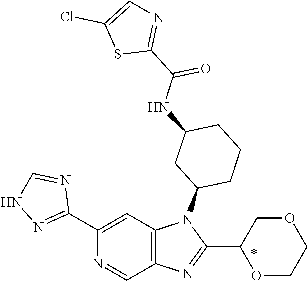 [SMILES: O=C(N[C@H]1CCC[C@@H](n2c(C3COCCO3)nc3cnc(-c4nc[nH]n4)cc32)C1)c1ncc(Cl)s1] | N-((1S,3R)-3-(2-(1,4-dioxan-2-yl)-6- (1H-1,2,4-triazol-3-yl)-1H- imidazo[4,5-c]pyridin-1- yl)cyclohexyl)-5-chlorothiazole-2- carboxamide (Diastereomer #1) |
| 178 | 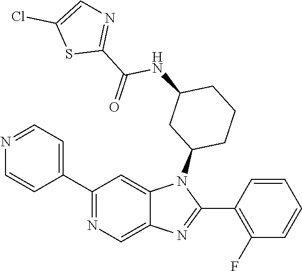 [SMILES: O=C(N[C@H]1CCC[C@@H](n2c(-c3ccccc3F)nc3cnc(-c4ccncc4)cc32)C1)c1ncc(Cl)s1] | 5-chloro-N-((1S,3R)-3-(2-(2- fluorophenyl)-6-(pyridin-4-yl)-1H- imidazo[4,5-c]pyridin-1- yl)cyclohexyl)thiazole-2-carboxamide |
| 179 | 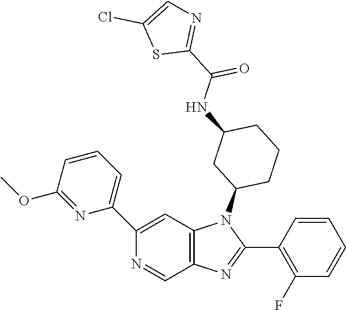 [SMILES: COc1cccc(-c2cc3c(cn2)nc(-c2ccccc2F)n3[C@@H]2CCC[C@H](NC(=O)c3ncc(Cl)s3)C2)n1] | 5-chloro-N-((1S,3R)-3-(2-(2- fluorophenyl)-6-(6-methoxypyridin- 2-yl)-1H-imidazo[4,5-c]pyridin-1- yl)cyclohexyl)thiazole-2-carboxamide |
| 180 | 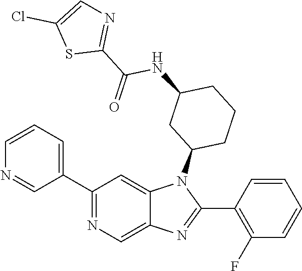 [SMILES: O=C(N[C@H]1CCC[C@@H](n2c(-c3ccccc3F)nc3cnc(-c4cccnc4)cc32)C1)c1ncc(Cl)s1] | 5-chloro-N-((1S,3R)-3-(2-(2- fluorophenyl)-6-(pyridin-3-yl)-1H- imidazo[4,5-c]pyridin-1- yl)cyclohexyl)thiazole-2-carboxamide |
| 181 | 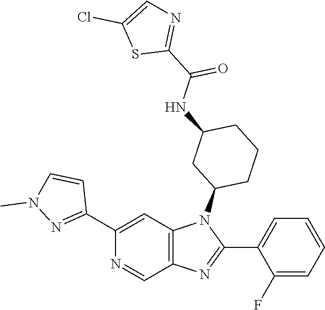 [SMILES: Cn1ccc(-c2cc3c(cn2)nc(-c2ccccc2F)n3[C@@H]2CCC[C@H](NC(=O)c3ncc(Cl)s3)C2)n1] | 5-chloro-N-((1S,3R)-3-(2-(2- fluorophenyl)-6-(1-methyl-1H- pyrazol-3-yl)-1H-imidazo[4,5- c]pyridin-1-yl)cyclohexyl)thiazole-2- carboxamide |
| 182 | 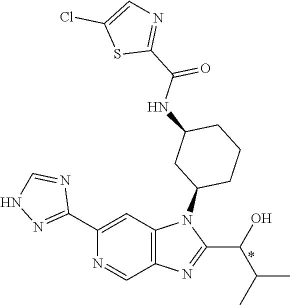 [SMILES: CC(C)C(O)c1nc2cnc(-c3nc[nH]n3)cc2n1[C@@H]1CCC[C@H](NC(=O)c2ncc(Cl)s2)C1] | 5-chloro-N-((1S,3R)-3-(2-(1- hydroxy-2-methylpropyl)-6-(1H- 1,2,4-triazol-3-yl)-1H-imidazo[4,5- c]pyridin-1-yl)cyclohexyl)thiazole-2- carboxamide (Diastereomer #2) |
| 183 | 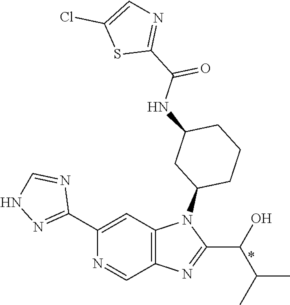 [SMILES: CC(C)C(O)c1nc2cnc(-c3nc[nH]n3)cc2n1[C@@H]1CCC[C@H](NC(=O)c2ncc(Cl)s2)C1] | 5-chloro-N-((1S,3R)-3-(2-(1- hydroxy-2-methylpropyl)-6-(1H- 1,2,4-triazol-3-yl)-1H-imidazo[4,5- c]pyridin-1-yl)cyclohexyl)thiazole-2- carboxamide (Diastereomer #1) |
| 184 | 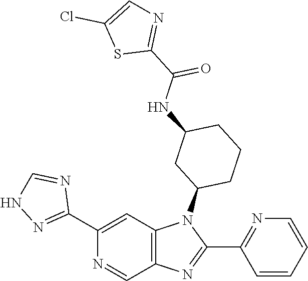 [SMILES: O=C(N[C@H]1CCC[C@@H](n2c(-c3ccccn3)nc3cnc(-c4nc[nH]n4)cc32)C1)c1ncc(Cl)s1] | 5-chloro-N-((1S,3R)-3-(2-(pyridin-2- yl)-6-(1H-1,2,4-triazol-3-yl)-1H- imidazo[4,5-c]pyridin-1- yl)cyclohexyl)thiazole-2-carboxamide |
| 185 | 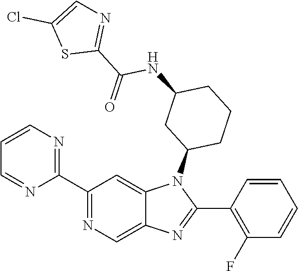 [SMILES: O=C(N[C@H]1CCC[C@@H](n2c(-c3ccccc3F)nc3cnc(-c4ncccn4)cc32)C1)c1ncc(Cl)s1] | 5-chloro-N-((1S,3R)-3-(2-(2- fluorophenyl)-6-(pyrimidin-2-yl)-1H- imidazo[4,5-c]pyridin-1- yl)cyclohexyl)thiazole-2-carboxamide |
| 186 | 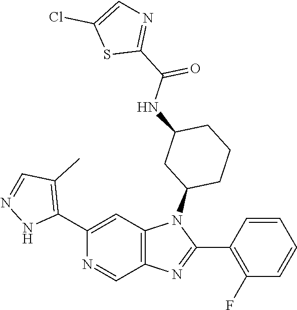 [SMILES: Cc1cn[nH]c1-c1cc2c(cn1)nc(-c1ccccc1F)n2[C@@H]1CCC[C@H](NC(=O)c2ncc(Cl)s2)C1] | 5-chloro-N-((1S,3R)-3-(2-(2- fluorophenyl)-6-(4-methyl-1H- pyrazol-5-yl)-1H-imidazo[4,5- c]pyridin-1-yl)cyclohexyl)thiazole-2- carboxamide |
| 187 | 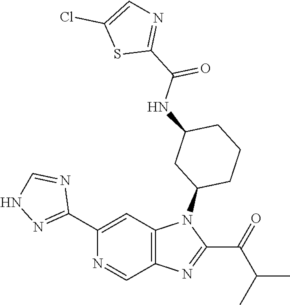 [SMILES: CC(C)C(=O)c1nc2cnc(-c3nc[nH]n3)cc2n1[C@@H]1CCC[C@H](NC(=O)c2ncc(Cl)s2)C1] | 5-chloro-N-((1S,3R)-3-(2-isobutyryl- 6-(1H-1,2,4-triazol-3-yl)-1H- imidazo[4,5-c]pyridin-1- yl)cyclohexyl)thiazole-2-carboxamide |
| 188 | 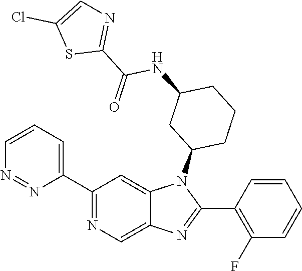 [SMILES: O=C(N[C@H]1CCC[C@@H](n2c(-c3ccccc3F)nc3cnc(-c4cccnn4)cc32)C1)c1ncc(Cl)s1] | 5-chloro-N-((1S,3R)-3-(2-(2- fluorophenyl)-6-(pyridazin-3-yl)-1H- imidazo[4,5-c]pyridin-1- yl)cyclohexyl)thiazole-2-carboxamide |
| 189 | 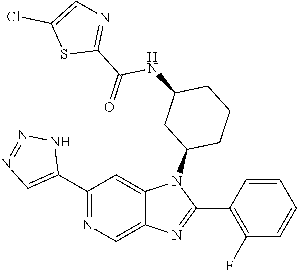 [SMILES: O=C(N[C@H]1CCC[C@@H](n2c(-c3ccccc3F)nc3cnc(-c4cnn[nH]4)cc32)C1)c1ncc(Cl)s1] | 5-chloro-N-((1S,3R)-3-(2-(2- fluorophenyl)-6-(1H-1,2,3-triazol-5- yl)-1H-imidazo[4,5-c]pyridin-1- yl)cyclohexyl)thiazole-2-carboxamide |
| 190 | 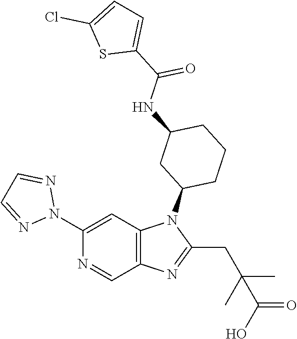 [SMILES: CC(C)(Cc1nc2cnc(-n3nccn3)cc2n1[C@@H]1CCC[C@H](NC(=O)c2ccc(Cl)s2)C1)C(=O)O] | 3-(1-((1R,3S)-3-(5-chlorothiophene- 2-carboxamido)cyclohexyl)-6-(2H- 1,2,3-triazol-2-yl)-1H-imidazo[4,5- c]pyridin-2-yl)-2,2- dimethylpropanoic acid |
| 191 | 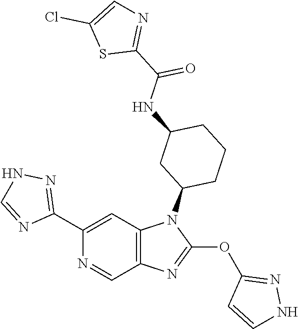 [SMILES: O=C(N[C@H]1CCC[C@@H](n2c(Oc3cc[nH]n3)nc3cnc(-c4nc[nH]n4)cc32)C1)c1ncc(Cl)s1] | N-((1S,3R)-3-(2-((1H-pyrazol-3- yl)oxy)-6-(1H-1,2,4-triazol-3-yl)-1H- imidazo[4,5-c]pyridin-1- yl)cyclohexyl)-5-chlorothiazole-2- carboxamide |
| 192 | 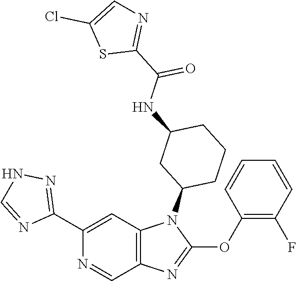 [SMILES: O=C(N[C@H]1CCC[C@@H](n2c(Oc3ccccc3F)nc3cnc(-c4nc[nH]n4)cc32)C1)c1ncc(Cl)s1] | 5-chloro-N-((1S,3R)-3-(2-(2- fluorophenoxy)-6-(1H-1,2,4-triazol-3- yl)-1H-imidazo[4,5-c]pyridin-1- yl)cyclohexyl)thiazole-2-carboxamide |
| 193 | 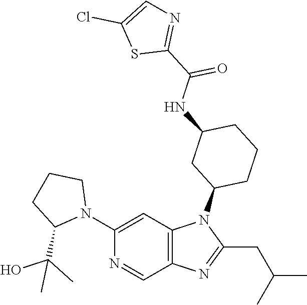 [SMILES: CC(C)Cc1nc2cnc(N3CCC[C@H]3C(C)(C)O)cc2n1[C@@H]1CCC[C@H](NC(=O)c2ncc(Cl)s2)C1] | 5-chloro-N-((1S,3R)-3-(6-((S)-2-(2- hydroxypropan-2-yl)pyrrolidin-1-yl)- 2-isobutyl-1H-imidazo[4,5-c]pyridin- 1-yl)cyclohexyl)thiazole-2- carboxamide |
| 194 | 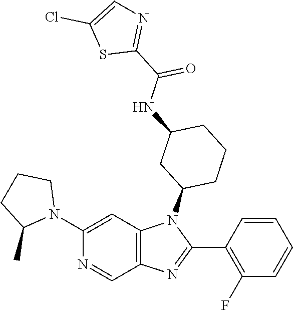 [SMILES: C[C@H]1CCCN1c1cc2c(cn1)nc(-c1ccccc1F)n2[C@@H]1CCC[C@H](NC(=O)c2ncc(Cl)s2)C1] | 5-chloro-N-((1S,3R)-3-(2-(2- fluorophenyl)-6-((S)-2- methylpyrrolidin-1-yl)-1H- imidazo[4,5-c]pyridin-1- yl)cyclohexyl)thiazole-2-carboxamide |
| 195 | 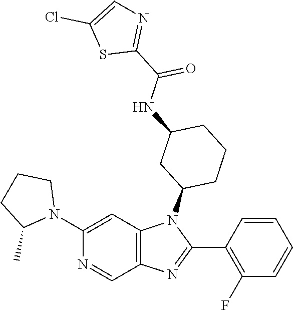 [SMILES: C[C@@H]1CCCN1c1cc2c(cn1)nc(-c1ccccc1F)n2[C@@H]1CCC[C@H](NC(=O)c2ncc(Cl)s2)C1] | 5-chloro-N-((1S,3R)-3-(2-(2- fluorophenyl)-6-((R)-2- methylpyrrolidin-1-yl)-1H- imidazo[4,5-c]pyridin-1- yl)cyclohexyl)thiazole-2-carboxamide |
| 196 | 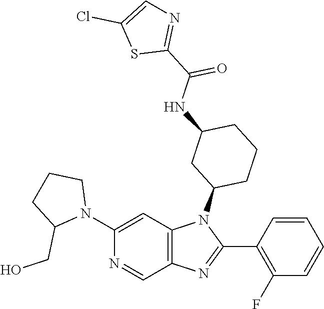 [SMILES: O=C(N[C@H]1CCC[C@@H](n2c(-c3ccccc3F)nc3cnc(N4CCCC4CO)cc32)C1)c1ncc(Cl)s1] | 5-chloro-N-((1S,3R)-3-(2-(2- fluorophenyl)-6-(2- (hydroxymethyl)pyrrolidin-1-yl)-1H- imidazo[4,5-c]pyridin-1- yl)cyclohexyl)thiazole-2-carboxamide |
| 197 | 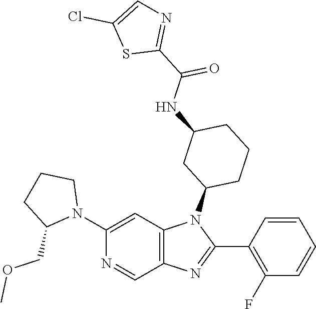 [SMILES: COC[C@@H]1CCCN1c1cc2c(cn1)nc(-c1ccccc1F)n2[C@@H]1CCC[C@H](NC(=O)c2ncc(Cl)s2)C1] | 5-chloro-N-((1S,3R)-3-(2-(2- fluorophenyl)-6-((S)-2- (methoxymethyl)pyrrolidin-1-yl)-1H- imidazo[4,5-c]pyridin-1- yl)cyclohexyl)thiazole-2-carboxamide |
| 198 | 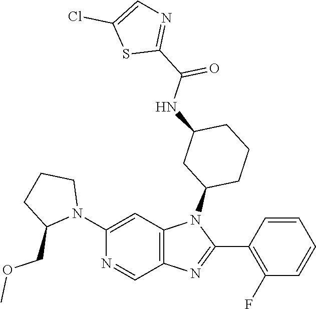 [SMILES: COC[C@H]1CCCN1c1cc2c(cn1)nc(-c1ccccc1F)n2[C@@H]1CCC[C@H](NC(=O)c2ncc(Cl)s2)C1] | 5-chloro-N-((1S,3R)-3-(2-(2- fluorophenyl)-6-((R)-2- (methoxymethyl)pyrrolidin-1-yl)-1H- imidazo[4,5-c]pyridin-1- yl)cyclohexyl)thiazole-2-carboxamide |
| 199 | 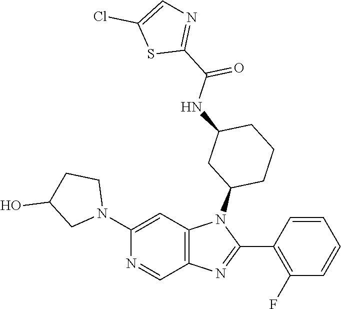 [SMILES: O=C(N[C@H]1CCC[C@@H](n2c(-c3ccccc3F)nc3cnc(N4CCC(O)C4)cc32)C1)c1ncc(Cl)s1] | 5-chloro-N-((1S,3R)-3-(2-(2- fluorophenyl)-6-(3- hydroxypyrrolidin-1-yl)-1H- imidazo[4,5-c]pyridin-1- yl)cyclohexyl)thiazole-2-carboxamide |
| 200 | 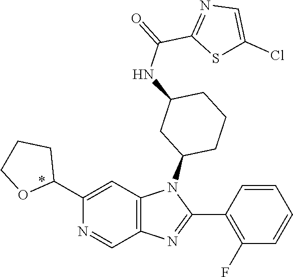 [SMILES: O=C(N[C@H]1CCC[C@@H](n2c(-c3ccccc3F)nc3cnc(C4CCCO4)cc32)C1)c1ncc(Cl)s1] | 5-chloro-N-((1S,3R)-3-(2-(2- fluorophenyl)-6-(tetrahydrofuran-2- yl)-1H-imidazo[4,5-c]pyridin-1- yl)cyclohexyl)thiazole-2-carboxamide (Diastereomer #2) |
| 201 | 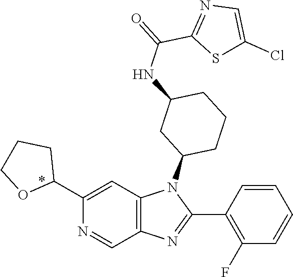 [SMILES: O=C(N[C@H]1CCC[C@@H](n2c(-c3ccccc3F)nc3cnc(C4CCCO4)cc32)C1)c1ncc(Cl)s1] | 5-chloro-N-((1S,3R)-3-(2-(2- fluorophenyl)-6-(tetrahydrofuran-2- yl)-1H-imidazo[4,5-c]pyridin-1- yl)cyclohexyl)thiazole-2-carboxamide (Diastereomer #1) |
| 202 | 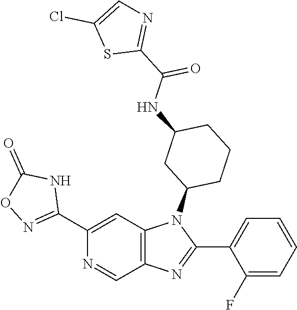 [SMILES: O=C(N[C@H]1CCC[C@@H](n2c(-c3ccccc3F)nc3cnc(-c4noc(=O)[nH]4)cc32)C1)c1ncc(Cl)s1] | 5-chloro-N-((1S,3R)-3-(2-(2- fluorophenyl)-6-(5-oxo-4,5-dihydro- 1,2,4-oxadiazol-3-yl)-1H- imidazo[4,5-c]pyridin-1- yl)cyclohexyl)thiazole-2-carboxamide |
| 203 | 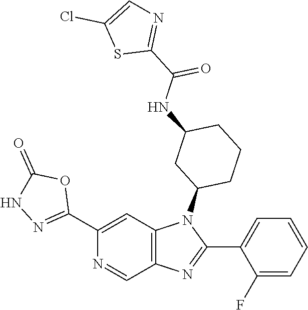 [SMILES: O=C(N[C@H]1CCC[C@@H](n2c(-c3ccccc3F)nc3cnc(-c4n[nH]c(=O)o4)cc32)C1)c1ncc(Cl)s1] | 5-chloro-N-((1S,3R)-3-(2-(2- fluorophenyl)-6-(5-oxo-4,5-dihydro- 1,3,4-oxadiazol-2-yl)-1H- imidazo[4,5-c]pyridin-1- yl)cyclohexyl)thiazole-2-carboxamide |
| 204 | 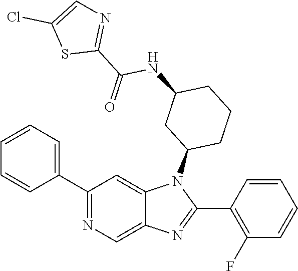 [SMILES: O=C(N[C@H]1CCC[C@@H](n2c(-c3ccccc3F)nc3cnc(-c4ccccc4)cc32)C1)c1ncc(Cl)s1] | 5-chloro-N-((1S,3R)-3-(2-(2- fluorophenyl)-6-phenyl-1H- imidazo[4,5-c]pyridin-1- yl)cyclohexyl)thiazole-2-carboxamide |
| 205 | 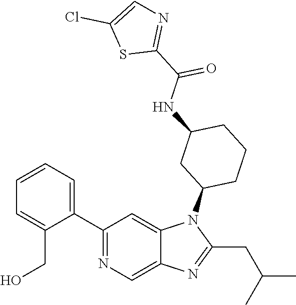 [SMILES: CC(C)Cc1nc2cnc(-c3ccccc3CO)cc2n1[C@@H]1CCC[C@H](NC(=O)c2ncc(Cl)s2)C1] | 5-chloro-N-((1S,3R)-3-(6-(2- (hydroxymethyl)phenyl)-2-isobutyl- 1H-imidazo[4,5-c]pyridin-1- yl)cyclohexyl)thiazole-2-carboxamide |
| 206 | 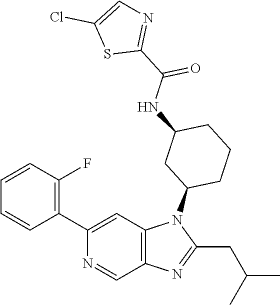 [SMILES: CC(C)Cc1nc2cnc(-c3ccccc3F)cc2n1[C@@H]1CCC[C@H](NC(=O)c2ncc(Cl)s2)C1] | 5-chloro-N-((1S,3R)-3-(6-(2- fluorophenyl)-2-isobutyl-1H- imidazo[4,5-c]pyridin-1- yl)cyclohexyl)thiazole-2-carboxamide |
| 207 | 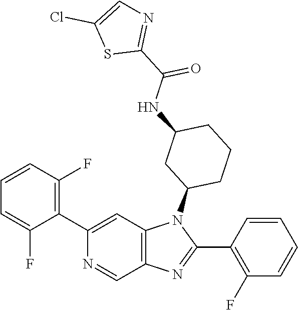 [SMILES: O=C(N[C@H]1CCC[C@@H](n2c(-c3ccccc3F)nc3cnc(-c4c(F)cccc4F)cc32)C1)c1ncc(Cl)s1] | 5-chloro-N-((1S,3R)-3-(6-(2,6- difluorophenyl)-2-(2-fluorophenyl)- 1H-imidazo[4,5-c]pyridin-1- yl)cyclohexyl)thiazole-2-carboxamide |
| 208 | 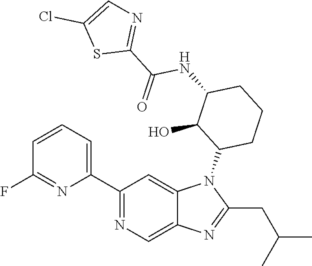 | 5-chloro-N-((1R,2S,3S)-3-(6-(6- fluoropyridin-2-yl)-2-isobutyl-1H- imidazo[4,5-c]pyridin-1-yl)-2- hydroxycyclohexyl)thiazole-2- carboxamide |
| 209 | 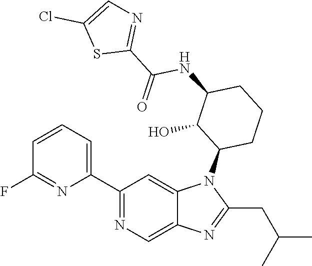 [SMILES: CC(C)Cc1nc2cnc(-c3cccc(F)n3)cc2n1[C@@H]1CCC[C@H](NC(=O)c2ncc(Cl)s2)[C@H]1O] | 5-chloro-N-((1S,2R,3R)-3-(6-(6- fluoropyridin-2-yl)-2-isobutyl-1H- imidazo[4,5-c]pyridin-1-yl)-2- hydroxycyclohexyl)thiazole-2- carboxamide |
| 210 | 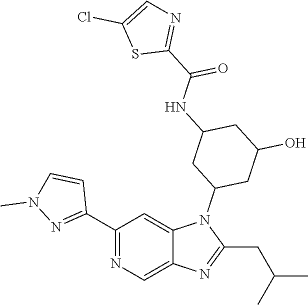 [SMILES: CC(C)Cc1nc2cnc(-c3ccn(C)n3)cc2n1C1CC(O)CC(NC(=O)c2ncc(Cl)s2)C1] | 5-chloro-N-(3-hydroxy-5-(2-isobutyl- 6-(1-methyl-1H-pyrazol-3-yl)-1H- imidazo[4,5-c]pyridin-1- yl)cyclohexyl)thiazole-2- carboxamide(7) |
| 211 | 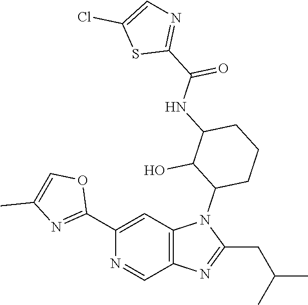 [SMILES: Cc1coc(-c2cc3c(cn2)nc(CC(C)C)n3C2CCCC(NC(=O)c3ncc(Cl)s3)C2O)n1] | 5-chloro-N-(2-hydroxy-3-(2-isobutyl- 6-(4-methyloxazol-2-yl)-1H- imidazo[4,5-c]pyridin-1- yl)cyclohexyl)thiazole-2- carboxamide(8) |
| 212 | 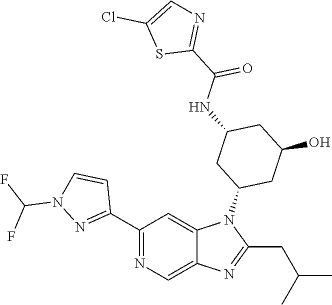 [SMILES: CC(C)Cc1nc2cnc(-c3ccn(C(F)F)n3)cc2n1[C@H]1C[C@H](O)C[C@@H](NC(=O)c2ncc(Cl)s2)C1] | 5-chloro-N-((1S,3R,5R)-3-(6-(1- (difluoromethyl)-1H-pyrazol-3-yl)-2- isobutyl-1H-imidazo[4,5-c]pyridin-1- yl)-5-hydroxycyclohexyl)thiazole-2- carboxamide |
| 213 | 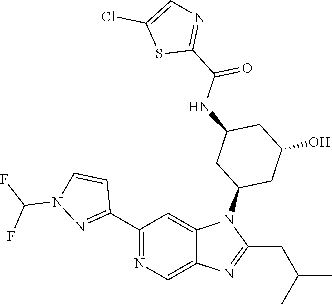 [SMILES: CC(C)Cc1nc2cnc(-c3ccn(C(F)F)n3)cc2n1[C@@H]1C[C@@H](O)C[C@H](NC(=O)c2ncc(Cl)s2)C1] | 5-chloro-N-((1R,3S,5R)-3-(6-(1- (difluoromethyl)-1H-pyrazol-3-yl)-2- isobutyl-1H-imidazo[4,5-c]pyridin-1- yl)-5-hydroxycyclohexyl)thiazole-2- carboxamide |
| 214 | 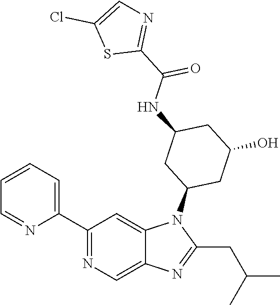 [SMILES: CC(C)Cc1nc2cnc(-c3ccccn3)cc2n1[C@@H]1C[C@@H](O)C[C@H](NC(=O)c2ncc(Cl)s2)C1] | 5-chloro-N-((1R,3S,5S)-3-hydroxy-5- (2-isobutyl-6-(pyridin-2-yl)-1H- imidazo[4,5-c]pyridin-1- yl)cyclohexyl)thiazole-2-carboxamide |
| 215 | 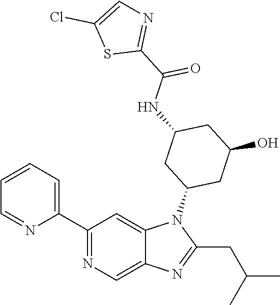 [SMILES: CC(C)Cc1nc2cnc(-c3ccccn3)cc2n1[C@H]1C[C@H](O)C[C@@H](NC(=O)c2ncc(Cl)s2)C1] | 5-chloro-N-((1S,3R,5R)-3-hydroxy- 5-(2-isobutyl-6-(pyridin-2-yl)-1H- imidazo[4,5-c]pyridin-1- yl)cyclohexyl)thiazole-2-carboxamide |
| 216 | 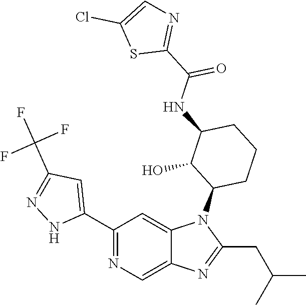 [SMILES: CC(C)Cc1nc2cnc(-c3cc(C(F)(F)F)n[nH]3)cc2n1[C@@H]1CCC[C@H](NC(=O)c2ncc(Cl)s2)[C@H]1O] | 5-chloro-N-((1S,2R,3R)-2-hydroxy- 3-(2-isobutyl-6-(3-(trifluoromethyl)- 1H-pyrazol-5-yl)-1H-imidazo[4,5- c]pyridin-1-yl)cyclohexyl)thiazole-2- carboxamide |
| 217 | 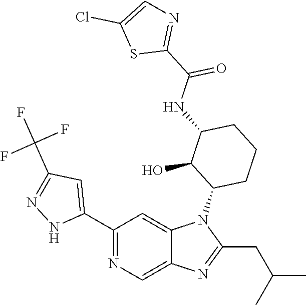 [SMILES: CC(C)Cc1nc2cnc(-c3cc(C(F)(F)F)n[nH]3)cc2n1[C@H]1CCC[C@@H](NC(=O)c2ncc(Cl)s2)[C@@H]1O] | 5-chloro-N-((1R,2S,3S)-2-hydroxy-3- (2-isobutyl-6-(3-(trifluoromethyl)- 1H-pyrazol-5-yl)-1H-imidazo[4,5- c]pyridin-1-yl)cyclohexyl)thiazole-2- carboxamide |
| 218 | 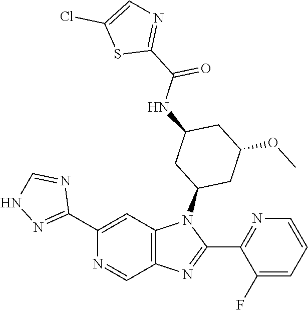 [SMILES: CO[C@H]1C[C@H](NC(=O)c2ncc(Cl)s2)C[C@H](n2c(-c3ncccc3F)nc3cnc(-c4nc[nH]n4)cc32)C1] | 5-chloro-N-((1R,3S,5S)-3-(2-(2- fluorophenyl)-6-(1H-1,2,4-triazol-3- yl)-1H-imidazo[4,5-c]pyridin-1-yl)-5- methoxycyclohexyl)thiazole-2- carboxamide |
| 219 | 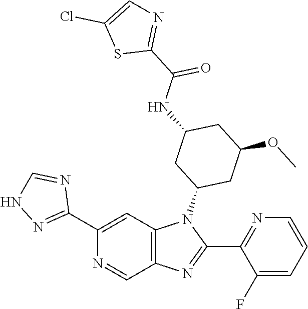 [SMILES: CO[C@@H]1C[C@@H](NC(=O)c2ncc(Cl)s2)C[C@@H](n2c(-c3ncccc3F)nc3cnc(-c4nc[nH]n4)cc32)C1] | 5-chloro-N-((1S,3R,5R)-3-(2-(2- fluorophenyl)-6-(1H-1,2,4-triazol-3- yl)-1H-imidazo[4,5-c]pyridin-1-yl)-5- methoxycyclohexyl)thiazole-2- carboxamide |
| 220 | 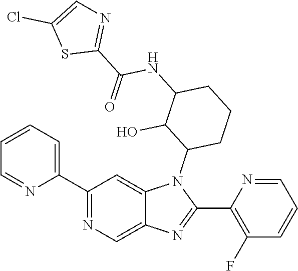 [SMILES: O=C(NC1CCCC(n2c(-c3ncccc3F)nc3cnc(-c4ccccn4)cc32)C1O)c1ncc(Cl)s1] | 5-chloro-N-(3-(2-(3-fluoropyridin-2- yl)-6-(pyridin-2-yl)-1H-imidazo[4,5- c]pyridin-1-yl)-2- hydroxycyclohexyl)thiazole-2- carboxamide(8) |
| 221 | 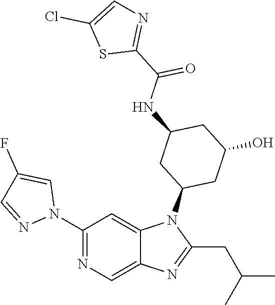 [SMILES: CC(C)Cc1nc2cnc(-n3cc(F)cn3)cc2n1[C@@H]1C[C@@H](O)C[C@H](NC(=O)c2ncc(Cl)s2)C1] | 5-chloro-N-((1R,3S,5S)-3-(6-(4- fluoro-1H-pyrazol-1-yl)-2-isobutyl- 1H-imidazo[4,5-c]pyridin-1-yl)-5- hydroxycyclohexyl)thiazole-2- carboxamide |
| 222 | 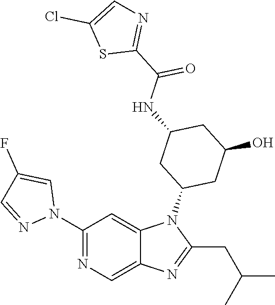 [SMILES: CC(C)Cc1nc2cnc(-n3cc(F)cn3)cc2n1[C@H]1C[C@H](O)C[C@@H](NC(=O)c2ncc(Cl)s2)C1] | 5-chloro-N-((1S,3R,5R)-3-(6-(4- fluoro-1H-pyrazol-1-yl)-2-isobutyl- 1H-imidazo[4,5-c]pyridin-1-yl)-5- hydroxycyclohexyl)thiazole-2- carboxamide |
| 223 | 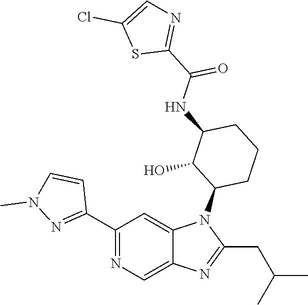 [SMILES: CC(C)Cc1nc2cnc(-c3ccn(C)n3)cc2n1[C@@H]1CCC[C@H](NC(=O)c2ncc(Cl)s2)[C@H]1O] | 5-chloro-N-((1S,2R,3R)-2-hydroxy- 3-(2-isobutyl-6-(1-methyl-1H- pyrazol-3-yl)-1H-imidazo[4,5- c]pyridin-1-yl)cyclohexyl)thiazole-2- carboxamide |
| 224 | 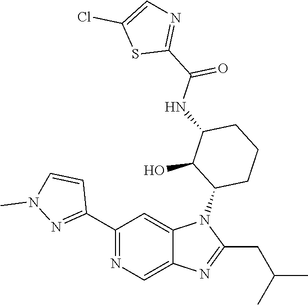 [SMILES: CC(C)Cc1nc2cnc(-c3ccn(C)n3)cc2n1[C@H]1CCC[C@@H](NC(=O)c2ncc(Cl)s2)[C@@H]1O] | 5-chloro-N-((1R,2S,3S)-2-hydroxy-3- (2-isobutyl-6-(1-methyl-1H-pyrazol- 3-yl)-1H-imidazo[4,5-c]pyridin-1- yl)cyclohexyl)thiazole-2-carboxamide |
| 225 | 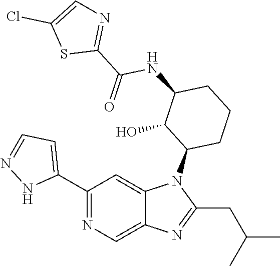 [SMILES: CC(C)Cc1nc2cnc(-c3ccn[nH]3)cc2n1[C@@H]1CCC[C@H](NC(=O)c2ncc(Cl)s2)[C@H]1O] | 5-chloro-N-((1S,2R,3R)-2-hydroxy- 3-(2-isobutyl-6-(1H-pyrazol-5-yl)- 1H-imidazo[4,5-c]pyridin-1- yl)cyclohexyl)thiazole-2-carboxamide |
| 226 | 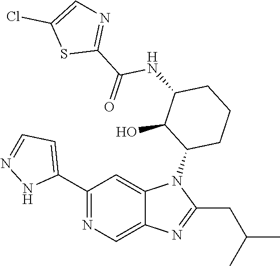 [SMILES: CC(C)Cc1nc2cnc(-c3ccn[nH]3)cc2n1[C@H]1CCC[C@@H](NC(=O)c2ncc(Cl)s2)[C@@H]1O] | 5-chloro-N-((1R,2S,3S)-2-hydroxy-3- (2-isobutyl-6-(1H-pyrazol-5-yl)-1H- imidazo[4,5-c]pyridin-1- yl)cyclohexyl)thiazole-2-carboxamide |
| 227 | 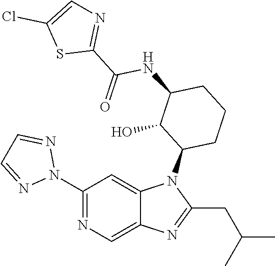 [SMILES: CC(C)Cc1nc2cnc(-n3nccn3)cc2n1[C@@H]1CCC[C@H](NC(=O)c2ncc(Cl)s2)[C@H]1O] | 5-chloro-N-((1S,2R,3R)-2-hydroxy- 3-(2-isobutyl-6-(2H-1,2,3-triazol-2- yl)-1H-imidazo[4,5-c]pyridin-1- yl)cyclohexyl)thiazole-2-carboxamide |
| 228 | 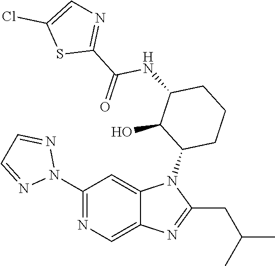 [SMILES: CC(C)Cc1nc2cnc(-n3nccn3)cc2n1[C@H]1CCC[C@@H](NC(=O)c2ncc(Cl)s2)[C@@H]1O] | 5-chloro-N-((1R,2S,3S)-2-hydroxy-3- (2-isobutyl-6-(2H-1,2,3-triazol-2-yl)- 1H-imidazo[4,5-c]pyridin-1- yl)cyclohexyl)thiazole-2-carboxamide |
| 229 | 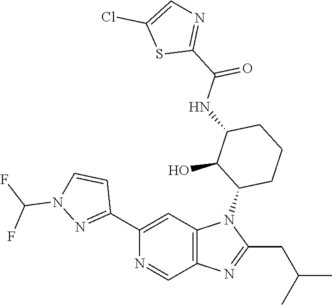 [SMILES: CC(C)Cc1nc2cnc(-c3ccn(C(F)F)n3)cc2n1[C@H]1CCC[C@@H](NC(=O)c2ncc(Cl)s2)[C@@H]1O] | 5-chloro-N-((1R,2S,3S)-3-(6-(1- (difluoromethyl)-1H-pyrazol-3-yl)-2- isobutyl-1H-imidazo[4,5-c]pyridin-1- yl)-2-hydroxycyclohexyl)thiazole-2- carboxamide |
| 230 | 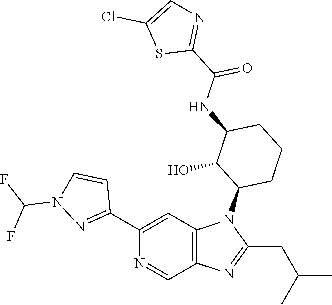 [SMILES: CC(C)Cc1nc2cnc(-c3ccn(C(F)F)n3)cc2n1[C@@H]1CCC[C@H](NC(=O)c2ncc(Cl)s2)[C@H]1O] | 5-chloro-N-((1S,2R,3R)-3-(6-(1- (difluoromethyl)-1H-pyrazol-3-yl)-2- isobutyl-1H-imidazo[4,5-c]pyridin-1- yl)-2-hydroxycyclohexyl)thiazole-2- carboxamide |
| 231 | 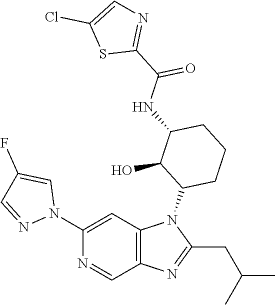 | 5-chloro-N-((1R,2S,3S)-3-(6-(4- fluoro-1H-pyrazol-1-yl)-2-isobutyl- 1H-imidazo[4,5-c]pyridin-1-yl)-2- hydroxycyclohexyl)thiazole-2- carboxamide |
| 232 | 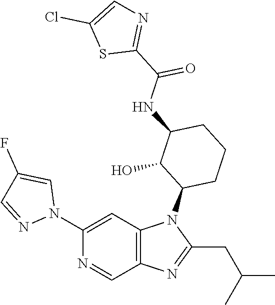 [SMILES: CC(C)Cc1nc2cnc(-n3cc(F)cn3)cc2n1[C@@H]1CCC[C@H](NC(=O)c2ncc(Cl)s2)[C@H]1O] | 5-chloro-N-((1S,2R,3R)-3-(6-(4- fluoro-1H-pyrazol-1-yl)-2-isobutyl- 1H-imidazo[4,5-c]pyridin-1-yl)-2- hydroxycyclohexyl)thiazole-2- carboxamide |
| 233 | 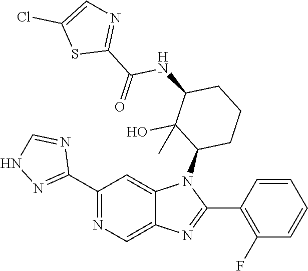 [SMILES: CC1(O)[C@@H](NC(=O)c2ncc(Cl)s2)CCC[C@H]1n1c(-c2ccccc2F)nc2cnc(-c3nc[nH]n3)cc21] | 5-chloro-N-((1S,3R)-3-(2-(2- fluorophenyl)-6-(1H-1,2,4-triazol-3- yl)-1H-imidazo[4,5-c]pyridin-1-yl)-2- hydroxy-2- methylcyclohexyl)thiazole-2- carboxamide |
| 234 | 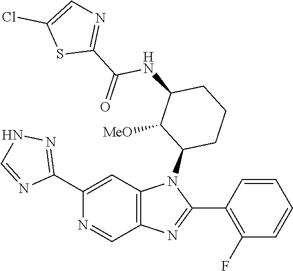 [SMILES: CO[C@@H]1[C@@H](NC(=O)c2ncc(Cl)s2)CCC[C@H]1n1c(-c2ccccc2F)nc2cnc(-c3nc[nH]n3)cc21] | 5-chloro-N-((1S,2R,3R)-3-(2-(2- fluorophenyl)-6-(1H-1,2,4-triazol-3- yl)-1H-imidazo[4,5-c]pyridin-1-yl)-2- methoxycyclohexyl)thiazole-2- carboxamide |
| 235 | 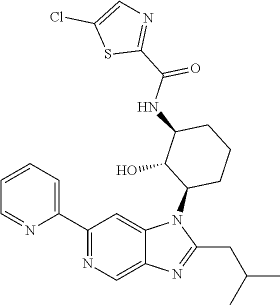 [SMILES: CC(C)Cc1nc2cnc(-c3ccccn3)cc2n1[C@@H]1CCC[C@H](NC(=O)c2ncc(Cl)s2)[C@H]1O] | 5-chloro-N-((1S,2R,3R)-2-hydroxy- 3-(2-isobutyl-6-(pyridin-2-yl)-1H- imidazo[4,5-c]pyridin-1- yl)cyclohexyl)thiazole-2-carboxamide |
| 236 | 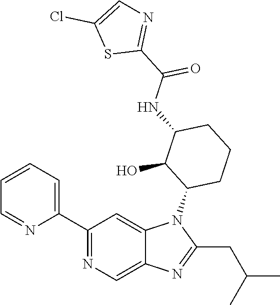 [SMILES: CC(C)Cc1nc2cnc(-c3ccccn3)cc2n1[C@H]1CCC[C@@H](NC(=O)c2ncc(Cl)s2)[C@@H]1O] | 5-chloro-N-((1R,2S,3S)-2-hydroxy-3- (2-isobutyl-6-(pyridin-2-yl)-1H- imidazo[4,5-c]pyridin-1- yl)cyclohexyl)thiazole-2-carboxamide |
| 237 | 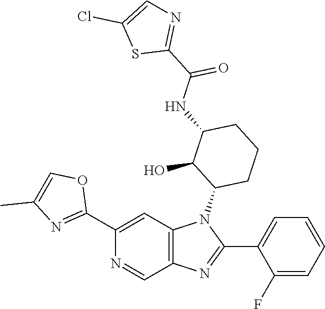 [SMILES: Cc1coc(-c2cc3c(cn2)nc(-c2ccccc2F)n3[C@H]2CCC[C@@H](NC(=O)c3ncc(Cl)s3)[C@@H]2O)n1] | 5-chloro-N-((1R,2S,3S)-3-(2-(2- fluorophenyl)-6-(4-methyloxazol-2- yl)-1H-imidazo[4,5-c]pyridin-1-yl)-2- hydroxycyclohexyl)thiazole-2- carboxamide |
| 238 | 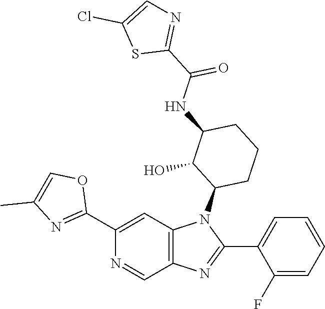 [SMILES: Cc1coc(-c2cc3c(cn2)nc(-c2ccccc2F)n3[C@@H]2CCC[C@H](NC(=O)c3ncc(Cl)s3)[C@H]2O)n1] | 5-chloro-N-((1S,2R,3R)-3-(2-(2- fluorophenyl)-6-(4-methyloxazol-2- yl)-1H-imidazo[4,5-c]pyridin-1-yl)-2- hydroxycyclohexyl)thiazole-2- carboxamide |
| 239 | 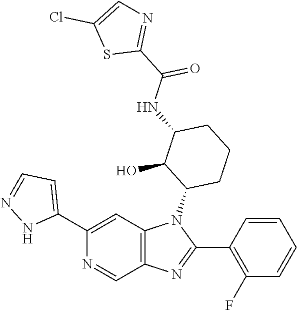 [SMILES: O=C(N[C@@H]1CCC[C@H](n2c(-c3ccccc3F)nc3cnc(-c4ccn[nH]4)cc32)[C@H]1O)c1ncc(Cl)s1] | 5-chloro-N-((1R,2S,3S)-3-(2-(2- fluorophenyl)-6-(1H-pyrazol-5-yl)- 1H-imidazo[4,5-c]pyridin-1-yl)-2- hydroxycyclohexyl)thiazole-2- carboxamide |
| 240 | 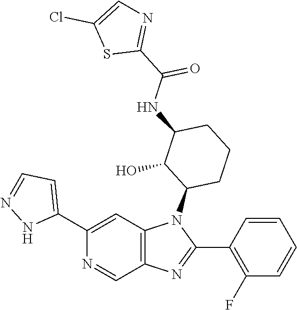 [SMILES: O=C(N[C@H]1CCC[C@@H](n2c(-c3ccccc3F)nc3cnc(-c4ccn[nH]4)cc32)[C@@H]1O)c1ncc(Cl)s1] | 5-chloro-N-((1S,2R,3R)-3-(2-(2- fluorophenyl)-6-(1H-pyrazol-5-yl)- 1H-imidazo[4,5-c]pyridin-1-yl)-2- hydroxycyclohexyl)thiazole-2- carboxamide |
| 241 | 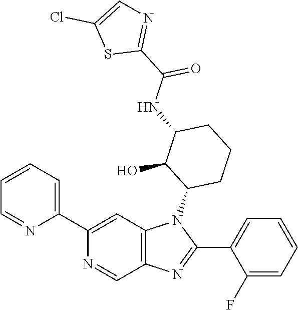 [SMILES: O=C(N[C@@H]1CCC[C@H](n2c(-c3ccccc3F)nc3cnc(-c4ccccn4)cc32)[C@H]1O)c1ncc(Cl)s1] | 5-chloro-N-((1R,2S,3S)-3-(2-(2- fluorophenyl)-6-(pyridin-2-yl)-1H- imidazo[4,5-c]pyridin-1-yl)-2- hydroxycyclohexyl)thiazole-2- carboxamide |
| 242 | 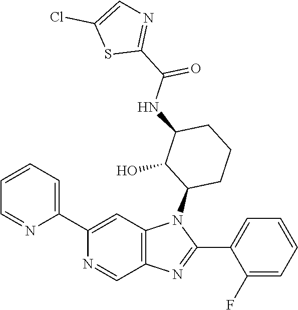 [SMILES: O=C(N[C@H]1CCC[C@@H](n2c(-c3ccccc3F)nc3cnc(-c4ccccn4)cc32)[C@@H]1O)c1ncc(Cl)s1] | 5-chloro-N-((1S,2R,3R)-3-(2-(2- fluorophenyl)-6-(pyridin-2-yl)-1H- imidazo[4,5-c]pyridin-1-yl)-2- hydroxycyclohexyl)thiazole-2- carboxamide |
| 243 | 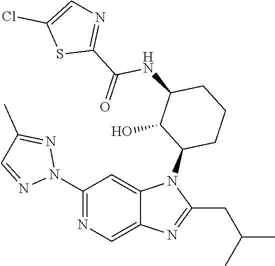 [SMILES: Cc1cnn(-c2cc3c(cn2)nc(CC(C)C)n3[C@@H]2CCC[C@H](NC(=O)c3ncc(Cl)s3)[C@H]2O)n1] | 5-chloro-N-((1S,2R,3R)-2-hydroxy- 3-(2-isobutyl-6-(4-methyl-2H-1,2,3- triazol-2-yl)-1H-imidazo[4,5- c]pyridin-1-yl)cyclohexyl)thiazole-2- carboxamide |
| 244 | 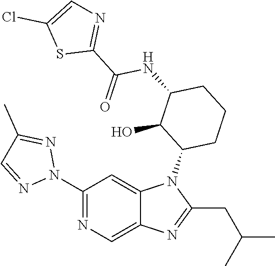 [SMILES: Cc1cnn(-c2cc3c(cn2)nc(CC(C)C)n3[C@H]2CCC[C@@H](NC(=O)c3ncc(Cl)s3)[C@@H]2O)n1] | 5-chloro-N-((1R,2S,3S)-2-hydroxy-3- (2-isobutyl-6-(4-methyl-2H-1,2,3- triazol-2-yl)-1H-imidazo[4,5- c]pyridin-1-yl)cyclohexyl)thiazole-2- carboxamide |
| 245 | 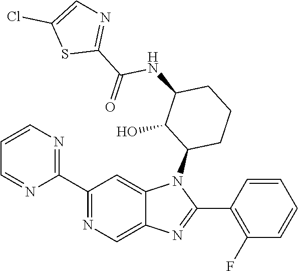 [SMILES: O=C(N[C@H]1CCC[C@@H](n2c(-c3ccccc3F)nc3cnc(-c4ncccn4)cc32)[C@@H]1O)c1ncc(Cl)s1] | 5-chloro-N-((1S,2R,3R)-3-(2-(2- fluorophenyl)-6-(pyrimidin-2-yl)-1H- imidazo[4,5-c]pyridin-1-yl)-2- hydroxycyclohexyl)thiazole-2- carboxamide |
| 246 | 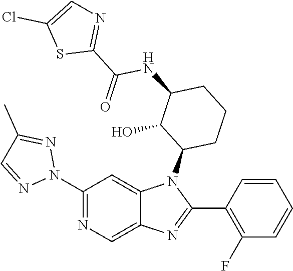 [SMILES: Cc1cnn(-c2cc3c(cn2)nc(-c2ccccc2F)n3[C@@H]2CCC[C@H](NC(=O)c3ncc(Cl)s3)[C@H]2O)n1] | 5-chloro-N-((1S,2R,3R)-3-(2-(2- fluorophenyl)-6-(4-methyl-2H-1,2,3- triazol-2-yl)-1H-imidazo[4,5- c]pyridin-1-yl)-2- hydroxycyclohexyl)thiazole-2- carboxamide |
| 247 | 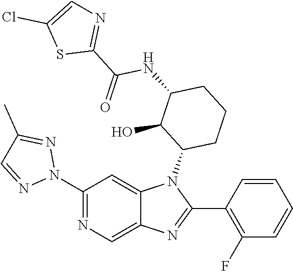 [SMILES: Cc1cnn(-c2cc3c(cn2)nc(-c2ccccc2F)n3[C@H]2CCC[C@@H](NC(=O)c3ncc(Cl)s3)[C@@H]2O)n1] | 5-chloro-N-((1R,2S,3S)-3-(2-(2- fluorophenyl)-6-(4-methyl-2H-1,2,3- triazol-2-yl)-1H-imidazo[4,5- c]pyridin-1-yl)-2- hydroxycyclohexyl)thiazole-2- carboxamide |
| 248 | 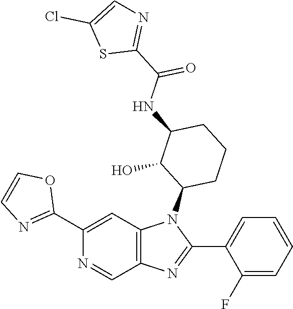 [SMILES: O=C(N[C@H]1CCC[C@@H](n2c(-c3ccccc3F)nc3cnc(-c4ncco4)cc32)[C@@H]1O)c1ncc(Cl)s1] | 5-chloro-N-((1S,2R,3R)-3-(2-(2- fluorophenyl)-6-(oxazol-2-yl)-1H- imidazo[4,5-c]pyridin-1-yl)-2- hydroxycyclohexyl)thiazole-2- carboxamide |
| 249 | 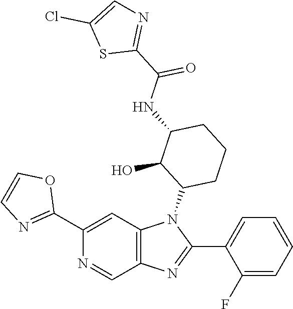 | 5-chloro-N-((1R,2S,3S)-3-(2-(2- fluorophenyl)-6-(oxazol-2-yl)-1H- imidazo[4,5-c]pyridin-1-yl)-2- hydroxycyclohexyl)thiazole-2- carboxamide |
| 250 | 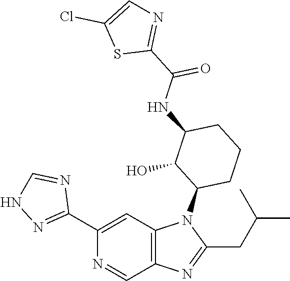 [SMILES: CC(C)Cc1nc2cnc(-c3nc[nH]n3)cc2n1[C@@H]1CCC[C@H](NC(=O)c2ncc(Cl)s2)[C@H]1O] | 5-chloro-N-((1S,2R,3R)-2-hydroxy- 3-(2-isobutyl-6-(1H-1,2,4-triazol-3- yl)-1H-imidazo[4,5-c]pyridin-1- yl)cyclohexyl)thiazole-2-carboxamide |
| 251 | 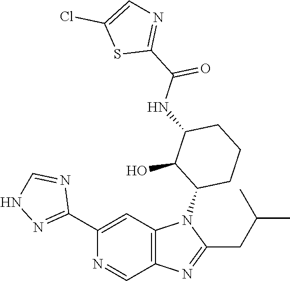 [SMILES: CC(C)Cc1nc2cnc(-c3nc[nH]n3)cc2n1[C@H]1CCC[C@@H](NC(=O)c2ncc(Cl)s2)[C@@H]1O] | 5-chloro-N-((1R,2S,3S)-2-hydroxy-3- (2-isobutyl-6-(1H-1,2,4-triazol-3-yl)- 1H-imidazo[4,5-c]pyridin-1- yl)cyclohexyl)thiazole-2-carboxamide |
| 252 | 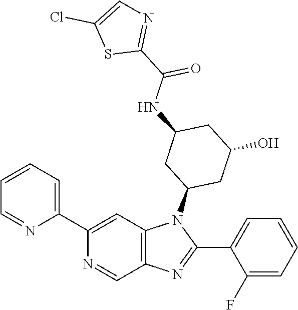 [SMILES: O=C(N[C@H]1C[C@H](O)C[C@@H](n2c(-c3ccccc3F)nc3cnc(-c4ccccn4)cc32)C1)c1ncc(Cl)s1] | 5-chloro-N-((1R,3S,5S)-3-(2-(2- fluorophenyl)-6-(pyridin-2-yl)-1H- imidazo[4,5-c]pyridin-1-yl)-5- hydroxycyclohexyl)thiazole-2- carboxamide |
| 253 | 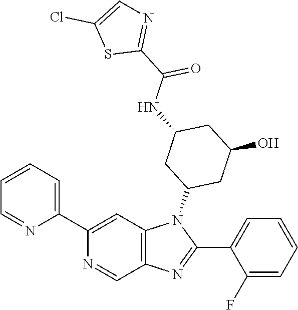 | 5-chloro-N-((1S,3R,5R)-3-(2-(2- fluorophenyl)-6-(pyridin-2-yl)-1H- imidazo[4,5-c]pyridin-1-yl)-5- hydroxycyclohexyl)thiazole-2- carboxamide |
| 254 | 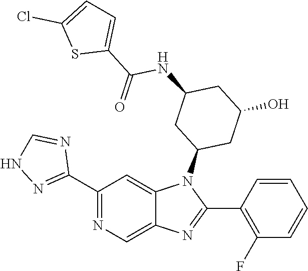 [SMILES: O=C(N[C@H]1C[C@H](O)C[C@@H](n2c(-c3ccccc3F)nc3cnc(-c4nc[nH]n4)cc32)C1)c1ccc(Cl)s1] | 5-chloro-N-((1R,3S,5S)-3-(2-(2- fluorophenyl)-6-(1H-1,2,4-triazol-3- yl)-1H-imidazo[4,5-c]pyridin-1-yl)-5- hydroxycyclohexyl)thiophene-2- carboxamide |
| 255 | 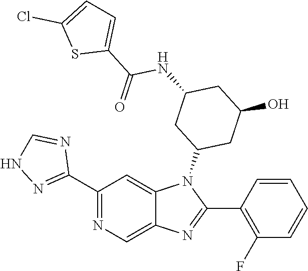 | 5-chloro-N-((1S,3R,5R)-3-(2-(2- fluorophenyl)-6-(1H-1,2,4-triazol-3- yl)-1H-imidazo[4,5-c]pyridin-1-yl)-5- hydroxycyclohexyl)thiophene-2- carboxamide |
| 256 | 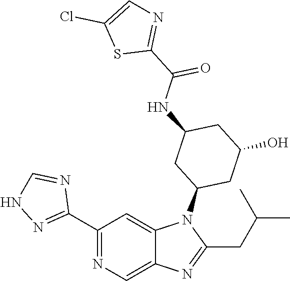 [SMILES: CC(C)Cc1nc2cnc(-c3nc[nH]n3)cc2n1[C@@H]1C[C@@H](O)C[C@H](NC(=O)c2ncc(Cl)s2)C1] | 5-chloro-N-((1R,3S,5S)-3-hydroxy-5- (2-isobutyl-6-(1H-1,2,4-triazol-3-yl)- 1H-imidazo[4,5-c]pyridin-1- yl)cyclohexyl)thiazole-2-carboxamide |
| 257 | 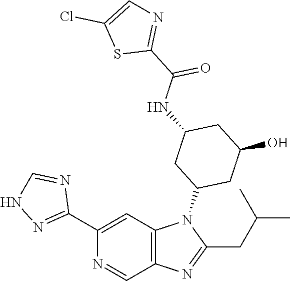 | 5-chloro-N-((1S,3R,5R)-3-hydroxy- 5-(2-isobutyl-6-(1H-1,2,4-triazol-3- yl)-1H-imidazo[4,5-c]pyridin-1- yl)cyclohexyl)thiazole-2-carboxamide |
| 258 | 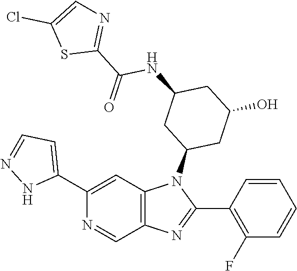 [SMILES: O=C(N[C@H]1C[C@H](O)C[C@@H](n2c(-c3ccccc3F)nc3cnc(-c4ccn[nH]4)cc32)C1)c1ncc(Cl)s1] | 5-chloro-N-((1R,3S,5S)-3-(2-(2- fluorophenyl)-6-(1H-pyrazol-5-yl)- 1H-imidazo[4,5-c]pyridin-1-yl)-5- hydroxycyclohexyl)thiazole-2- carboxamide |
| 259 | 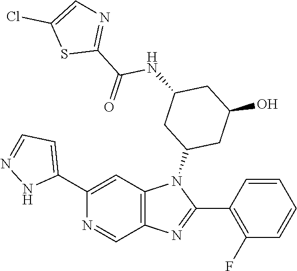 | 5-chloro-N-((1S,3R,5R)-3-(2-(2- fluorophenyl)-6-(1H-pyrazol-5-yl)- 1H-imidazo[4,5-c]pyridin-1-yl)-5- hydroxycyclohexyl)thiazole-2- carboxamide |
| 260 | 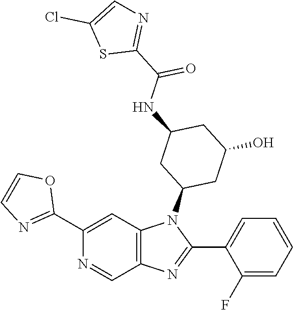 [SMILES: O=C(N[C@H]1C[C@H](O)C[C@@H](n2c(-c3ccccc3F)nc3cnc(-c4ncco4)cc32)C1)c1ncc(Cl)s1] | 5-chloro-N-((1R,3S,5S)-3-(2-(2- fluorophenyl)-6-(oxazol-2-yl)-1H- imidazo[4,5-c]pyridin-1-yl)-5- hydroxycyclohexyl)thiazole-2- carboxamide |
| 261 | 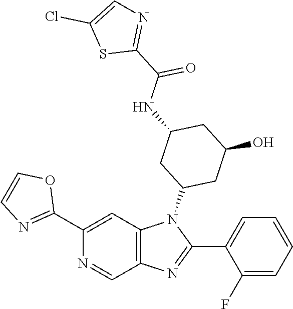 | 5-chloro-N-((1S,3R,5R)-3-(2-(2- fluorophenyl)-6-(oxazol-2-yl)-1H- imidazo[4,5-c]pyridin-1-yl)-5- hydroxycyclohexyl)thiazole-2- carboxamide |
| 262 | 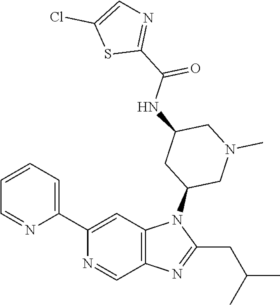 [SMILES: CC(C)Cc1nc2cnc(-c3ccccn3)cc2n1[C@H]1C[C@@H](NC(=O)c2ncc(Cl)s2)CN(C)C1] | 5-chloro-N-((3R,5S)-5-(2-isobutyl-6- (pyridin-2-yl)-1H-imidazo[4,5- c]pyridin-1-yl)-1-methylpiperidin-3- yl)thiazole-2-carboxamide |
| 263 | 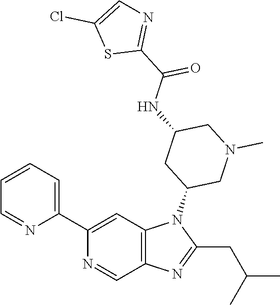 [SMILES: CC(C)Cc1nc2cnc(-c3ccccn3)cc2n1[C@@H]1C[C@H](NC(=O)c2ncc(Cl)s2)CN(C)C1] | 5-chloro-N-((3S,5R)-5-(2-isobutyl-6- (pyridin-2-yl)-1H-imidazo[4,5- c]pyridin-1-yl)-1-methylpiperidin-3- yl)thiazole-2-carboxamide |
| 264 | 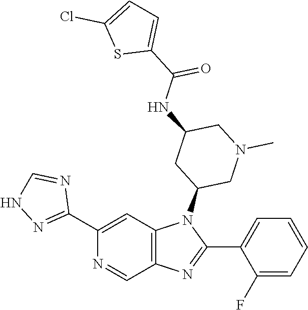 [SMILES: CN1C[C@H](NC(=O)c2ccc(Cl)s2)C[C@H](n2c(-c3ccccc3F)nc3cnc(-c4nc[nH]n4)cc32)C1] | 5-chloro-N-((3R,5S)-5-(2-(2- fluorophenyl)-6-(1H-1,2,4-triazol-3- yl)-1H-imidazo[4,5-c]pyridin-1-yl)-1- methylpiperidin-3-yl)thiophene-2- carboxamide |
| 265 | 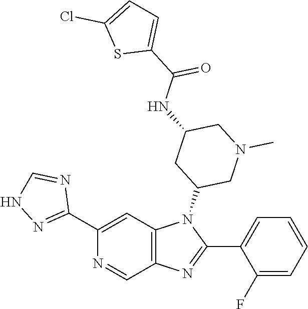 | 5-chloro-N-((3S,5R)-5-(2-(2- fluorophenyl)-6-(1H-1,2,4-triazol-3- yl)-1H-imidazo[4,5-c]pyridin-1-yl)-1- methylpiperidin-3-yl)thiophene-2- carboxamide |
| 300 | 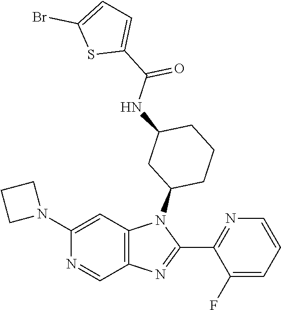 [SMILES: O=C(N[C@H]1CCC[C@@H](n2c(-c3ncccc3F)nc3cnc(N4CCC4)cc32)C1)c1ccc(Br)s1] | N-((1S,3R)-3-(6-(azetidin-1-yl)-2-(2- fluoropyridin-2-yl)-1H-imidazo[4,5- c]pyridin-1-yl)cyclohexyl)-5- bromothiophene-2-carboxamide |
| 301 | 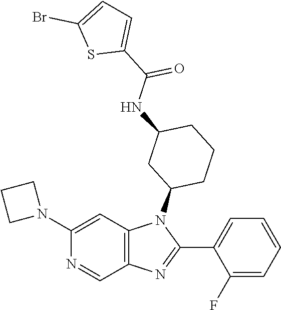 [SMILES: O=C(N[C@H]1CCC[C@@H](n2c(-c3ccccc3F)nc3cnc(N4CCC4)cc32)C1)c1ccc(Br)s1] | N-((1S,3R)-3-(6-(azetidin-1-yl)-2-(2- fluorophenyl)-1H-imidazo[4,5- c]pyridin-1-yl)cyclohexyl)-5- bromothiophene-2-carboxamide |
| 302 | 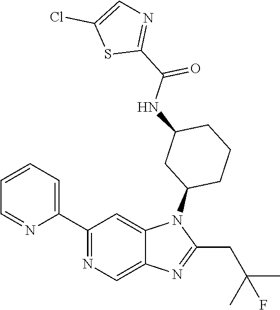 [SMILES: CC(C)(F)Cc1nc2cnc(-c3ccccn3)cc2n1[C@@H]1CCC[C@H](NC(=O)c2ncc(Cl)s2)C1] | 5-chloro-N-((1S,3R)-3-(2-(2-fluoro- 2-methylpropyl)-6-(pyridin-2-yl)-1H- imidazo[4,5-c]pyridin-1- yl)cyclohexyl)thiazole-2-carboxamide |
| 303 | 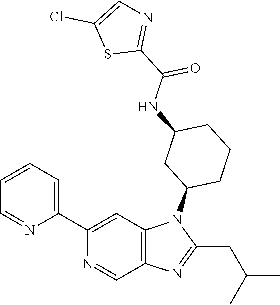 [SMILES: CC(C)Cc1nc2cnc(-c3ccccn3)cc2n1[C@@H]1CCC[C@H](NC(=O)c2ncc(Cl)s2)C1] | 5-chloro-N-((1S,3R)-3-(2-isobutyl-6- (pyridin-2-yl)-1H-imidazo[4,5- c]pyridin-1-yl)cyclohexyl)thiazole-2- carboxamide |
| 304 | 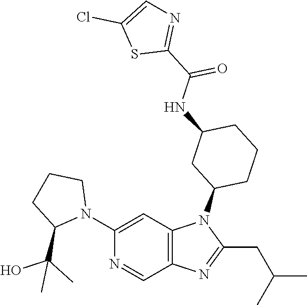 [SMILES: CC(C)Cc1nc2cnc(N3CCC[C@@H]3C(C)(C)O)cc2n1[C@@H]1CCC[C@H](NC(=O)c2ncc(Cl)s2)C1] | 5-chloro-N-((1S,3R)-3-(6-((R)-2-(2- hydroxypropan-2-yl)pyrrolidin-1-yl)- 2-isobutyl-1H-imidazo[4,5-c]pyridin- 1-yl)cyclohexyl)thiazole-2- carboxamide |
| 305 | 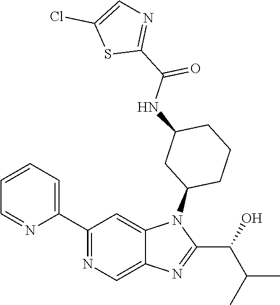 [SMILES: CC(C)[C@@H](O)c1nc2cnc(-c3ccccn3)cc2n1[C@@H]1CCC[C@H](NC(=O)c2ncc(Cl)s2)C1] | 5-chloro-N-((1S,3R)-3-(2-((R)-1- hydroxy-2-methylpropyl)-6-(pyridin- 2-yl)-1H-imidazo[4,5-c]pyridin-1- yl)cyclohexyl)thiazole-2-carboxamide |
| 306 | 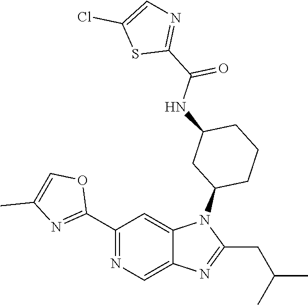 [SMILES: Cc1coc(-c2cc3c(cn2)nc(CC(C)C)n3[C@@H]2CCC[C@H](NC(=O)c3ncc(Cl)s3)C2)n1] | 5-chloro-N-((1S,3R)-3-(2-isobutyl-6- (4-methyloxazol-2-yl)-1H- imidazo[4,5-c]pyridin-1- yl)cyclohexyl)thiazole-2-carboxamide |
| 307 | 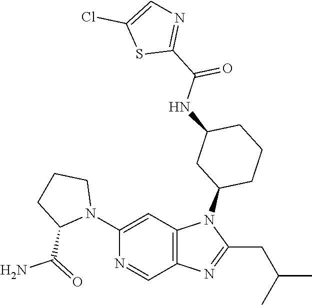 [SMILES: CC(C)Cc1nc2cnc(N3CCC[C@H]3C(N)=O)cc2n1[C@@H]1CCC[C@H](NC(=O)c2ncc(Cl)s2)C1] | N-((1S,3R)-3-(6-((S)-2- carbamoylpyrrolidin-1-yl)-2-isobutyl- 1H-imidazo[4,5-c]pyridin-1- yl)cyclohexyl)-5-chlorothiazole-2- carboxamide |
| 308 | 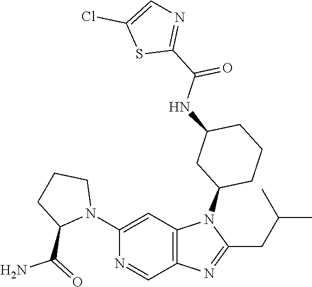 [SMILES: CC(C)Cc1nc2cnc(N3CCC[C@@H]3C(N)=O)cc2n1[C@@H]1CCC[C@H](NC(=O)c2ncc(Cl)s2)C1] | N-((1S,3R)-3-(6-((R)-2- carbamoylpyrrolidin-1-yl)-2-isobutyl- 1H-imidazo[4,5-c]pyridin-1- yl)cyclohexyl)-5-chlorothiazole-2- carboxamide |
| 309 | 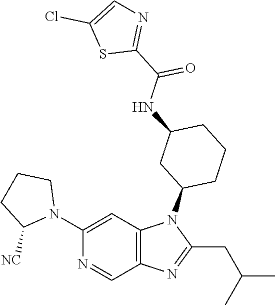 [SMILES: CC(C)Cc1nc2cnc(N3CCC[C@H]3C#N)cc2n1[C@@H]1CCC[C@H](NC(=O)c2ncc(Cl)s2)C1] | 5-chloro-N-((1S,3R)-3-(6-((S)-2- cyanopyrrolidin-1-yl)-2-isobutyl-1H- imidazo[4,5-c]pyridin-1- yl)cyclohexyl)thiazole-2-carboxamide |
| 310 | 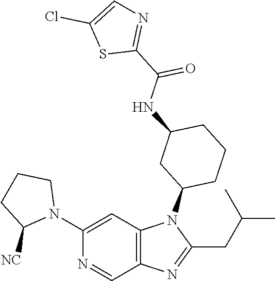 [SMILES: CC(C)Cc1nc2cnc(N3CCC[C@@H]3C#N)cc2n1[C@@H]1CCC[C@H](NC(=O)c2ncc(Cl)s2)C1] | 5-chloro-N-((1S,3R)-3-(6-((R)-2- cyanopyrrolidin-1-yl)-2-isobutyl-1H- imidazo[4,5-c]pyridin-1- yl)cyclohexyl)thiazole-2-carboxamide |
| 311 | 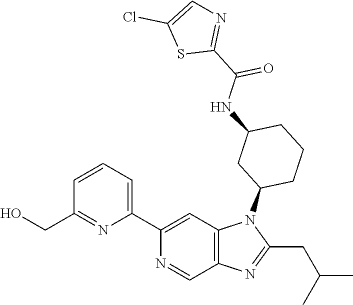 [SMILES: CC(C)Cc1nc2cnc(-c3cccc(CO)n3)cc2n1[C@@H]1CCC[C@H](NC(=O)c2ncc(Cl)s2)C1] | 5-chloro-N-((1S,3R)-3-(6-(6- (hydroxymethyl)pyridin-2-yl)-2- isobutyl-1H-imidazo[4,5-c]pyridin-1- yl)cyclohexyl)thiazole-2-carboxamide |
| 312 | 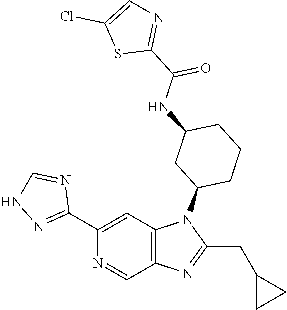 [SMILES: O=C(N[C@H]1CCC[C@@H](n2c(CC3CC3)nc3cnc(-c4nc[nH]n4)cc32)C1)c1ncc(Cl)s1] | 5-chloro-N-((1S,3R)-3-(2- (cyclopropylmethyl)-6-(1H-1,2,4- triazol-3-yl)-1H-imidazo[4,5- c]pyridin-1-yl)cyclohexyl)thiazole-2- carboxamide |
| 313 | 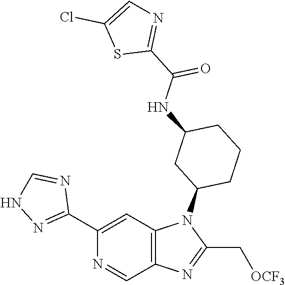 [SMILES: O=C(N[C@H]1CCC[C@@H](n2c(COC(F)(F)F)nc3cnc(-c4nc[nH]n4)cc32)C1)c1ncc(Cl)s1] | N-((1S,3R)-3-(6-(1H-1,2,4-triazol-3- yl)-2-((trifluoromethoxy)methyl)-1H- imidazo[4,5-c]pyridin-1- yl)cyclohexyl)-5-chlorothiazole-2- carboxamide |
| 314 | 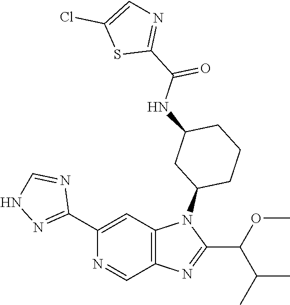 [SMILES: COC(c1nc2cnc(-c3nc[nH]n3)cc2n1[C@@H]1CCC[C@H](NC(=O)c2ncc(Cl)s2)C1)C(C)C] | 5-chloro-N-((1S,3R)-3-(2-(1- methoxy-2-methylpropyl)-6-(1H- 1,2,4-triazol-3-yl)-1H-imidazo[4,5- c]pyridin-1-yl)cyclohexyl)thiazole-2- carboxamide |
| 315 | 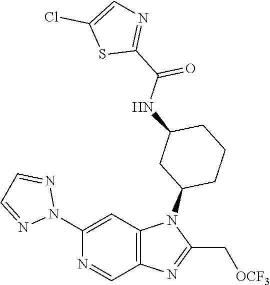 [SMILES: O=C(N[C@H]1CCC[C@@H](n2c(COC(F)(F)F)nc3cnc(-n4nccn4)cc32)C1)c1ncc(Cl)s1] | N-((1S,3R)-3-(6-(2H-1,2,3-triazol-2- yl)-2-((trifluoromethoxy)methyl)-1H- imidazo[4,5-c]pyridin-1- yl)cyclohexyl)-5-chlorothiazole-2- carboxamide |
| 316 | 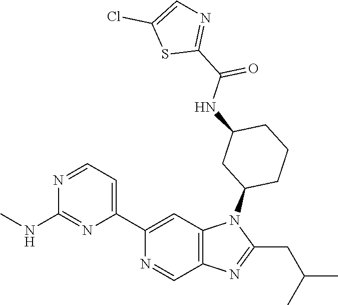 [SMILES: CNc1nccc(-c2cc3c(cn2)nc(CC(C)C)n3[C@@H]2CCC[C@H](NC(=O)c3ncc(Cl)s3)C2)n1] | 5-chloro-N-((1S,3R)-3-(2-isobutyl-6- (2-(methylamino)pyrimidin-4-yl)-1H- imidazo[4,5-c]pyridin-1- yl)cyclohexyl)thiazole-2-carboxamide |
| 317 | 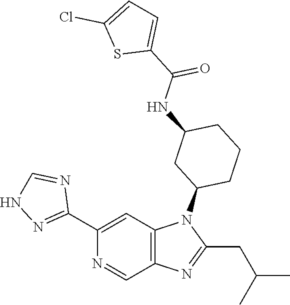 [SMILES: CC(C)Cc1nc2cnc(-c3nc[nH]n3)cc2n1[C@@H]1CCC[C@H](NC(=O)c2ccc(Cl)s2)C1] | 5-chloro-N-((1S,3R)-3-(2-isobutyl-6- (1H-1,2,4-triazol-3-yl)-1H- imidazo[4,5-c]pyridin-1- yl)cyclohexyl)thiophene-2- carboxamide |
| 318 | 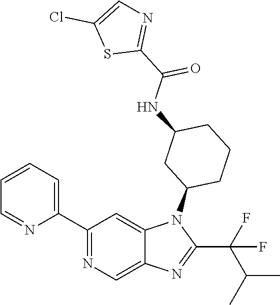 [SMILES: CC(C)C(F)(F)c1nc2cnc(-c3ccccn3)cc2n1[C@@H]1CCC[C@H](NC(=O)c2ncc(Cl)s2)C1] | 5-chloro-N-((1S,3R)-3-(2-(1,1- difluoro-2-methylpropyl)-6-(pyridin- 2-yl)-1H-imidazo[4,5-c]pyridin-1- yl)cyclohexyl)thiazole-2-carboxamide |
| 319 | 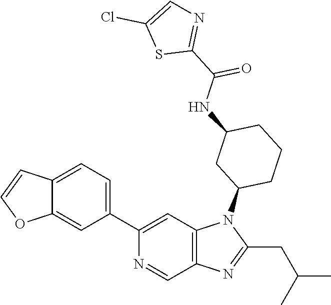 [SMILES: CC(C)Cc1nc2cnc(-c3ccc4ccoc4c3)cc2n1[C@@H]1CCC[C@H](NC(=O)c2ncc(Cl)s2)C1] | N-((1S,3R)-3-(6-(benzofuran-6-yl)-2- isobutyl-1H-imidazo[4,5-c]pyridin-1- yl)cyclohexyl)-5-chlorothiazole-2- carboxamide |
| 320 | 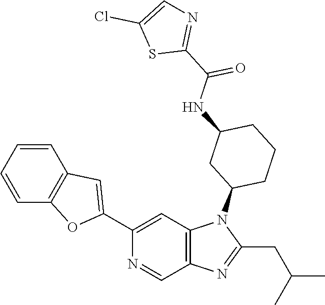 [SMILES: CC(C)Cc1nc2cnc(-c3cc4ccccc4o3)cc2n1[C@@H]1CCC[C@H](NC(=O)c2ncc(Cl)s2)C1] | N-((1S,3R)-3-(6-(benzofuran-2-yl)-2- isobutyl-1H-imidazo[4,5-c]pyridin-1- yl)cyclohexyl)-5-chlorothiazole-2- carboxamide |
| 321 | 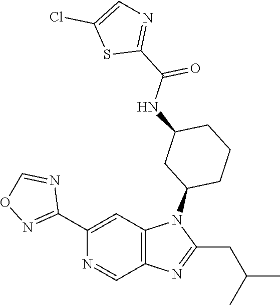 [SMILES: CC(C)Cc1nc2cnc(-c3ncon3)cc2n1[C@@H]1CCC[C@H](NC(=O)c2ncc(Cl)s2)C1] | 5-chloro-N-((1S,3R)-3-(2-isobutyl-6- (1,2,4-oxadiazol-3-yl)-1H- imidazo[4,5-c]pyridin-1- yl)cyclohexyl)thiazole-2-carboxamide |
| 322 | 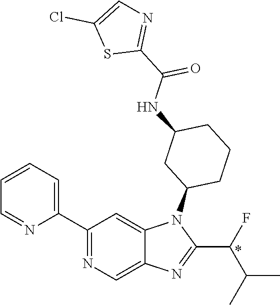 [SMILES: CC(C)C(F)c1nc2cnc(-c3ccccn3)cc2n1[C@@H]1CCC[C@H](NC(=O)c2ncc(Cl)s2)C1] | 5-chloro-N-((1S,3R)-3-(2-(1-fluoro- 2-methylpropyl)-6-(pyridin-2-yl)-1H- imidazo[4,5-c]pyridin-1- yl)cyclohexyl)thiazole-2-carboxamide |
| 323 | 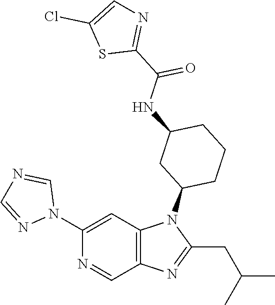 [SMILES: CC(C)Cc1nc2cnc(-n3cncn3)cc2n1[C@@H]1CCC[C@H](NC(=O)c2ncc(Cl)s2)C1] | 5-chloro-N-((1S,3R)-3-(2-isobutyl-6- (1H-1,2,4-triazol-1-yl)-1H- imidazo[4,5-c]pyridin-1- yl)cyclohexyl)thiazole-2-carboxamide |
| 324 | 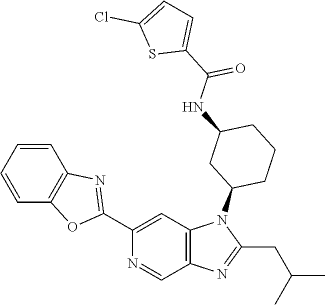 [SMILES: CC(C)Cc1nc2cnc(-c3nc4ccccc4o3)cc2n1[C@@H]1CCC[C@H](NC(=O)c2ccc(Cl)s2)C1] | N-((1S,3R)-3-(6-(benzo[d]oxazol-2- yl)-2-isobutyl-1H-imidazo[4,5- c]pyridin-1-yl)cyclohexyl)-5- chlorothiazole-2-carboxamide |
| 325 | 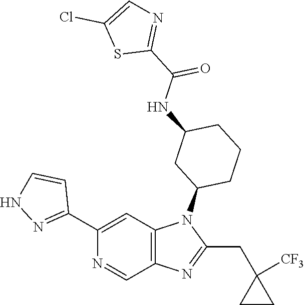 [SMILES: O=C(N[C@H]1CCC[C@@H](n2c(CC3(C(F)(F)F)CC3)nc3cnc(-c4cc[nH]n4)cc32)C1)c1ncc(Cl)s1] | N-((1S,3R)-3-(6-(1H-1,2,4-triazol-3- yl)-2-((1- (trifluoromethyl)cyclopropyl)methyl)- 1H-imidazo[4,5-c]pyridin-1- yl)cyclohexyl)-5-chlorothiazole-2- carboxamide |
| 326 | 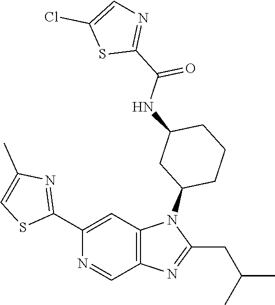 [SMILES: Cc1csc(-c2cc3c(cn2)nc(CC(C)C)n3[C@@H]2CCC[C@H](NC(=O)c3ncc(Cl)s3)C2)n1] | 5-chloro-N-((1S,3R)-3-(2-isobutyl-6- (4-methylthiazol-2-yl)-1H- imidazo[4,5-c]pyridin-1- yl)cyclohexyl)thiazole-2-carboxamide |
| 327 | 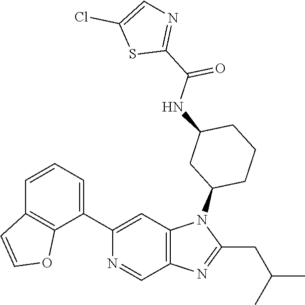 [SMILES: CC(C)Cc1nc2cnc(-c3cccc4ccoc34)cc2n1[C@@H]1CCC[C@H](NC(=O)c2ncc(Cl)s2)C1] | N-((1S,3R)-3-(6-(benzofuran-7-yl)-2- isobutyl-1H-imidazo[4,5-c]pyridin-1- yl)cyclohexyl)-5-chlorothiazole-2- carboxamide |
| 328 | 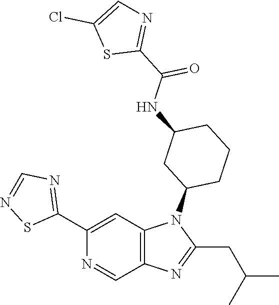 [SMILES: CC(C)Cc1nc2cnc(-c3ncns3)cc2n1[C@@H]1CCC[C@H](NC(=O)c2ncc(Cl)s2)C1] | 5-chloro-N-((1S,3R)-3-(2-isobutyl-6- (1,2,4-thiadiazol-5-yl)-1H- imidazo[4,5-c]pyridin-1- yl)cyclohexyl)thiazole-2-carboxamide |
| 329 | 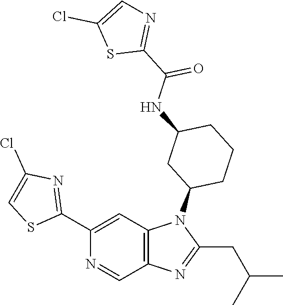 [SMILES: CC(C)Cc1nc2cnc(-c3nc(Cl)cs3)cc2n1[C@@H]1CCC[C@H](NC(=O)c2ncc(Cl)s2)C1] | 5-chloro-N-((1S,3R)-3-(6-(4- chlorothiazol-2-yl)-2-isobutyl-1H- imidazo[4,5-c]pyridin-1- yl)cyclohexyl)thiazole-2-carboxamide |
| 330 | 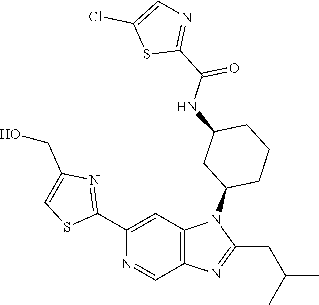 [SMILES: CC(C)Cc1nc2cnc(-c3nc(CO)cs3)cc2n1[C@@H]1CCC[C@H](NC(=O)c2ncc(Cl)s2)C1] | 5-chloro-N-((1S,3R)-3-(6-(4- (hydroxymethyl)thiazol-2-yl)-2- isobutyl-1H-imidazo[4,5-c]pyridin-1- yl)cyclohexyl)thiazole-2-carboxamide |
| 331 | 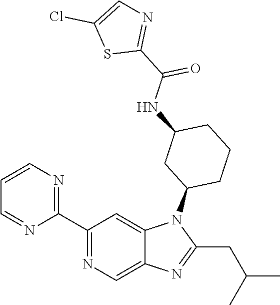 [SMILES: CC(C)Cc1nc2cnc(-c3ncccn3)cc2n1[C@@H]1CCC[C@H](NC(=O)c2ncc(Cl)s2)C1] | 5-chloro-N-((1S,3R)-3-(2-isobutyl-6- (pyrimidin-2-yl)-1H-imidazo[4,5- c]pyridin-1-yl)cyclohexyl)thiazole-2- carboxamide |
| 332 | 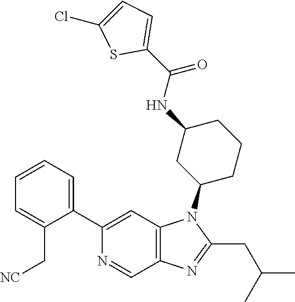 [SMILES: CC(C)Cc1nc2cnc(-c3ccccc3CC#N)cc2n1[C@@H]1CCC[C@H](NC(=O)c2ccc(Cl)s2)C1] | 5-chloro-N-((1S,3R)-3-(6-(2- (cyanomethyl)phenyl)-2-isobutyl-1H- imidazo[4,5-c]pyridin-1- yl)cyclohexyl)thiophene-2- carboxamide |
| 333 | 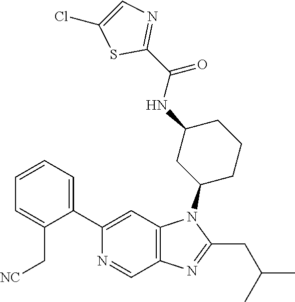 [SMILES: CC(C)Cc1nc2cnc(-c3ccccc3CC#N)cc2n1[C@@H]1CCC[C@H](NC(=O)c2ncc(Cl)s2)C1] | 5-chloro-N-((1S,3R)-3-(6-(2- (cyanomethyl)phenyl)-2-isobutyl-1H- imidazo[4,5-c]pyridin-1- yl)cyclohexyl)thiazole-2-carboxamide |
| 334 | 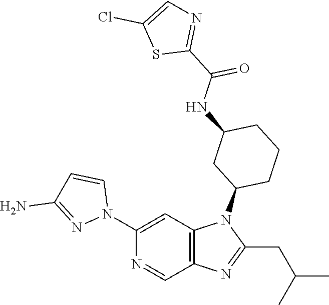 [SMILES: CC(C)Cc1nc2cnc(-n3ccc(N)n3)cc2n1[C@@H]1CCC[C@H](NC(=O)c2ncc(Cl)s2)C1] | N-((1S,3R)-3-(6-(3-amino-1H- pyrazol-1-yl)-2-isobutyl-1H- imidazo[4,5-c]pyridin-1- yl)cyclohexyl)-5-chlorothiazole-2- carboxamide |
| 335 | 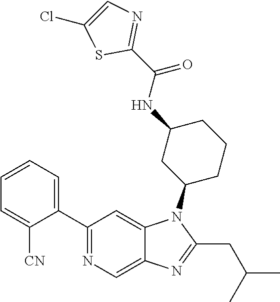 [SMILES: CC(C)Cc1nc2cnc(-c3ccccc3C#N)cc2n1[C@@H]1CCC[C@H](NC(=O)c2ncc(Cl)s2)C1] | 5-chloro-N-((1S,3R)-3-(6-(2- cyanophenyl)-2-isobutyl-1H- imidazo[4,5-c]pyridin-1- yl)cyclohexyl)thiazole-2-carboxamide |
| 336 | 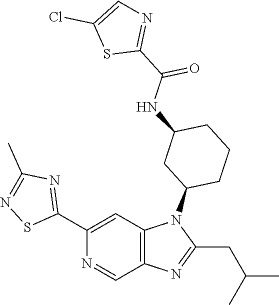 [SMILES: Cc1nsc(-c2cc3c(cn2)nc(CC(C)C)n3[C@@H]2CCC[C@H](NC(=O)c3ncc(Cl)s3)C2)n1] | 5-chloro-N-((1S,3R)-3-(2-isobutyl-6- (3-methyl-1,2,4-thiadiazol-5-yl)-1H- imidazo[4,5-c]pyridin-1- yl)cyclohexyl)thiazole-2-carboxamide |
| 337 | 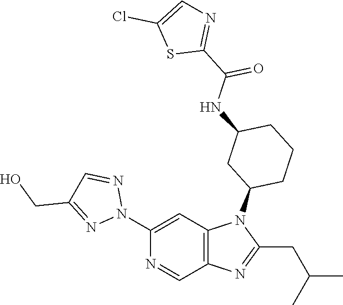 [SMILES: CC(C)Cc1nc2cnc(-n3ncc(CO)n3)cc2n1[C@@H]1CCC[C@H](NC(=O)c2ncc(Cl)s2)C1] | 5-chloro-N-((1S,3R)-3-(6-(4- (hydroxymethyl)-2H-1,2,3-triazol-2- yl)-2-isobutyl-1H-imidazo[4,5- c]pyridin-1-yl)cyclohexyl)thiazole-2- carboxamide |
| 338 | 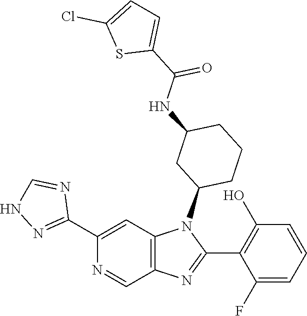 [SMILES: O=C(N[C@H]1CCC[C@@H](n2c(-c3c(O)cccc3F)nc3cnc(-c4nc[nH]n4)cc32)C1)c1ccc(Cl)s1] | 5-chloro-N-((1S,3R)-3-(2-(2-fluoro- 6-hydroxyphenyl)-6-(1H-1,2,4- triazol-3-yl)-1H-imidazo[4,5- c]pyridin-1-yl)cyclohexyl)thiophene- 2-carboxamide |
| 339 | 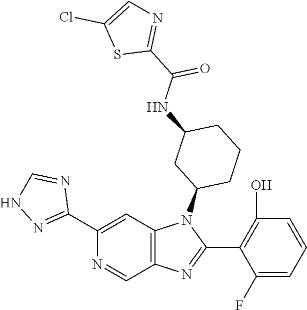 [SMILES: O=C(N[C@H]1CCC[C@@H](n2c(-c3c(O)cccc3F)nc3cnc(-c4nc[nH]n4)cc32)C1)c1ncc(Cl)s1] | 5-chloro-N-((1S,3R)-3-(2-(2-fluoro- 6-hydroxyphenyl)-6-(1H-1,2,4- triazol-3-yl)-1H-imidazo[4,5- c]pyridin-1-yl)cyclohexyl)thiazole-2- carboxamide |
| 340 | 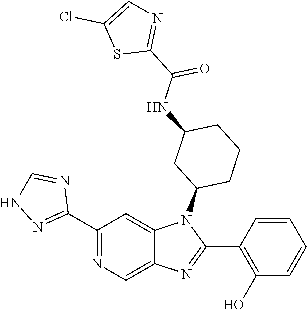 [SMILES: O=C(N[C@H]1CCC[C@@H](n2c(-c3ccccc3O)nc3cnc(-c4nc[nH]n4)cc32)C1)c1ncc(Cl)s1] | 5-chloro-N-((1S,3R)-3-(2-(2- hydroxyphenyl)-6-(1H-1,2,4-triazol- 3-yl)-1H-imidazo[4,5-c]pyridin-1- yl)cyclohexyl)thiazole-2-carboxamide |
| 341 | 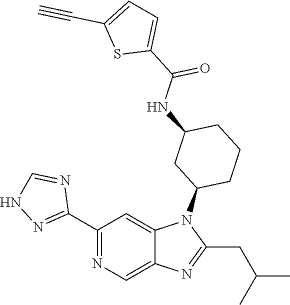 [SMILES: C#Cc1ccc(C(=O)N[C@H]2CCC[C@@H](n3c(CC(C)C)nc4cnc(-c5nc[nH]n5)cc43)C2)s1] | 5-ethynyl-N-((1S,3R)-3-(2-isobutyl- 6-(1H-1,2,4-triazol-3-yl)-1H- imidazo[4,5-c]pyridin-1- yl)cyclohexyl)thiophene-2- carboxamide |
| 342 | 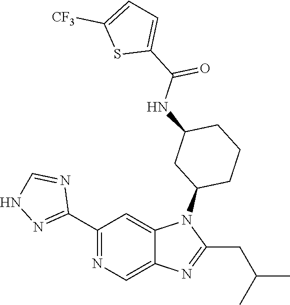 [SMILES: CC(C)Cc1nc2cnc(-c3nc[nH]n3)cc2n1[C@@H]1CCC[C@H](NC(=O)c2ccc(C(F)(F)F)s2)C1] | N-((1S,3R)-3-(2-isobutyl-6-(1H- 1,2,4-triazol-3-yl)-1H-imidazo[4,5- c]pyridin-1-yl)cyclohexyl)-5- (trifluoromethyl)thiophene-2- carboxamide |
| 343 | 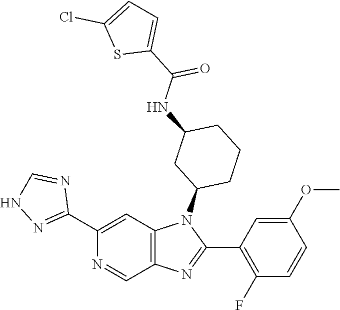 [SMILES: COc1ccc(F)c(-c2nc3cnc(-c4nc[nH]n4)cc3n2[C@@H]2CCC[C@H](NC(=O)c3ccc(Cl)s3)C2)c1] | 5-chloro-N-((1S,3R)-3-(2-(2-fluoro- 5-methoxyphenyl)-6-(1H-1,2,4- triazol-3-yl)-1H-imidazo[4,5- c]pyridin-1-yl)cyclohexyl)thiophene- 2-carboxamide |
| 344 | 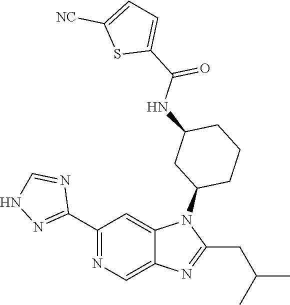 [SMILES: CC(C)Cc1nc2cnc(-c3nc[nH]n3)cc2n1[C@@H]1CCC[C@H](NC(=O)c2ccc(C#N)s2)C1] | 5-cyano-N-((1S,3R)-3-(2-isobutyl-6- (1H-1,2,4-triazol-3-yl)-1H- imidazo[4,5-c]pyridin-1- yl)cyclohexyl)thiophene-2- carboxamide |
| 345 | 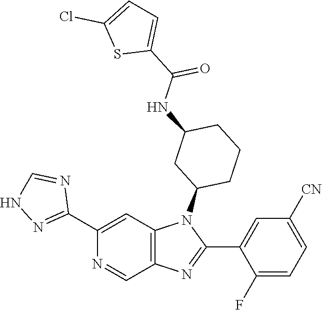 [SMILES: N#Cc1ccc(F)c(-c2nc3cnc(-c4nc[nH]n4)cc3n2[C@@H]2CCC[C@H](NC(=O)c3ccc(Cl)s3)C2)c1] | 5-chloro-N-((1S,3R)-3-(2-(5-cyano-2- fluorophenyl)-6-(1H-1,2,4-triazol-3- yl)-1H-imidazo[4,5-c]pyridin-1- yl)cyclohexyl)thiophene-2- carboxamide |
| 346 | 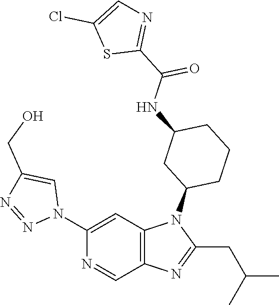 [SMILES: CC(C)Cc1nc2cnc(-n3cc(CO)nn3)cc2n1[C@@H]1CCC[C@H](NC(=O)c2ncc(Cl)s2)C1] | 5-chloro-N-((1S,3R)-3-(6-(4- (hydroxymethyl)-1H-1,2,3-triazol-1- yl)-2-isobutyl-1H-imidazo[4,5- c]pyridin-1-yl)cyclohexyl)thiazole-2- carboxamide |
| 347 | 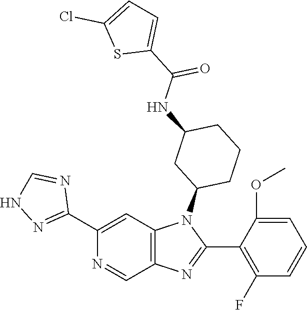 [SMILES: COc1cccc(F)c1-c1nc2cnc(-c3nc[nH]n3)cc2n1[C@@H]1CCC[C@H](NC(=O)c2ccc(Cl)s2)C1] | 5-chloro-N-((1S,3R)-3-(2-(2-fluoro- 6-methoxyphenyl)-6-(1H-1,2,4- triazol-3-yl)-1H-imidazo[4,5- c]pyridin-1-yl)cyclohexyl)thiophene- 2-carboxamide |
| 348 | 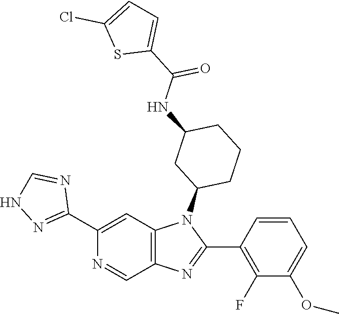 [SMILES: COc1cccc(-c2nc3cnc(-c4nc[nH]n4)cc3n2[C@@H]2CCC[C@H](NC(=O)c3ccc(Cl)s3)C2)c1F] | 5-chloro-N-((1S,3R)-3-(2-(2-fluoro- 3-methoxyphenyl)-6-(1H-1,2,4- triazol-3-yl)-1H-imidazo[4,5- c]pyridin-1-yl)cyclohexyl)thiophene- 2-carboxamide |
| 349 | 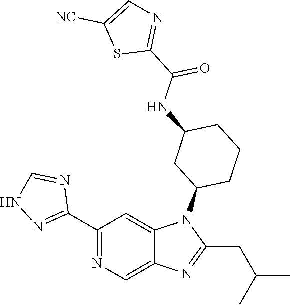 [SMILES: CC(C)Cc1nc2cnc(-c3nc[nH]n3)cc2n1[C@@H]1CCC[C@H](NC(=O)c2ncc(C#N)s2)C1] | 5-cyano-N-((1S,3R)-3-(2-isobutyl-6- (1H-1,2,4-triazol-3-yl)-1H- imidazo[4,5-c]pyridin-1- yl)cyclohexyl)thiazole-2-carboxamide |
| 350 | 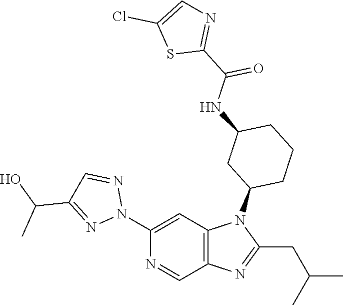 [SMILES: CC(C)Cc1nc2cnc(-n3ncc(C(C)O)n3)cc2n1[C@@H]1CCC[C@H](NC(=O)c2ncc(Cl)s2)C1] | 5-chloro-N-((1S,3R)-3-(6-(4-(1- hydroxyethyl)-2H-1,2,3-triazol-2-yl)- 2-isobutyl-1H-imidazo[4,5-c]pyridin- 1-yl)cyclohexyl)thiazole-2- carboxamide |
| 351 | 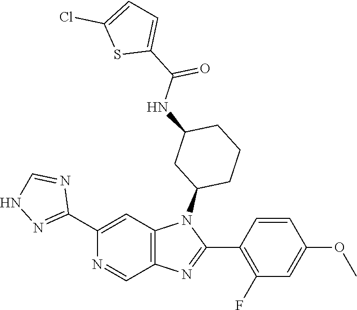 [SMILES: COc1ccc(-c2nc3cnc(-c4nc[nH]n4)cc3n2[C@@H]2CCC[C@H](NC(=O)c3ccc(Cl)s3)C2)c(F)c1] | 5-chloro-N-((1S,3R)-3-(2-(2-fluoro- 4-methoxyphenyl)-6-(1H-1,2,4- triazol-3-yl)-1H-imidazo[4,5- c]pyridin-1-yl)cyclohexyl)thiophene- 2-carboxamide |
| 352 | 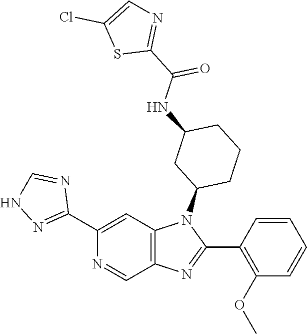 [SMILES: COc1ccccc1-c1nc2cnc(-c3nc[nH]n3)cc2n1[C@@H]1CCC[C@H](NC(=O)c2ncc(Cl)s2)C1] | 5-chloro-N-((1S,3R)-3-(2-(2- methoxyphenyl)-6-(1H-1,2,4-triazol- 3-yl)-1H-imidazo[4,5-c]pyridin-1- yl)cyclohexyl)thiazole-2-carboxamide |
| 353 | 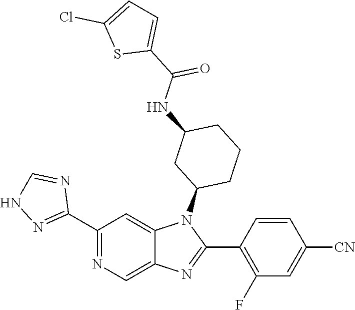 [SMILES: N#Cc1ccc(-c2nc3cnc(-c4nc[nH]n4)cc3n2[C@@H]2CCC[C@H](NC(=O)c3ccc(Cl)s3)C2)c(F)c1] | 5-chloro-N-((1S,3R)-3-(2-(4-cyano-2- fluorophenyl)-6-(1H-1,2,4-triazol-3- yl)-1H-imidazo[4,5-c]pyridin-1- yl)cyclohexyl)thiophene-2- carboxamide |
| 354 | 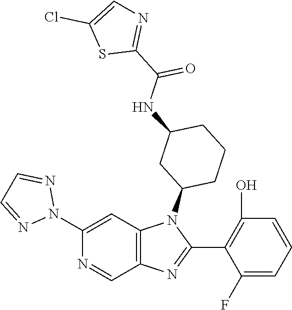 [SMILES: O=C(N[C@H]1CCC[C@@H](n2c(-c3c(O)cccc3F)nc3cnc(-n4nccn4)cc32)C1)c1ncc(Cl)s1] | 5-chloro-N-((1S,3R)-3-(2-(2-fluoro- 6-hydroxyphenyl)-6-(2H-1,2,3- triazol-2-yl)-1H-imidazo[4,5- c]pyridin-1-yl)cyclohexyl)thiazole-2- carboxamide |
| 355 | 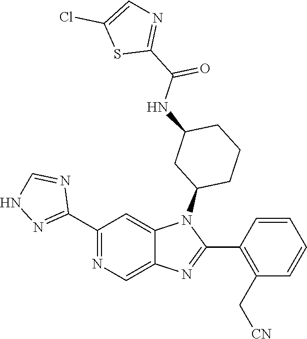 [SMILES: N#CCc1ccccc1-c1nc2cnc(-c3nc[nH]n3)cc2n1[C@@H]1CCC[C@H](NC(=O)c2ncc(Cl)s2)C1] | 5-chloro-N-((1S,3R)-3-(2-(2- (cyanomethyl)phenyl)-6-(1H-1,2,4- triazol-3-yl)-1H-imidazo[4,5- c]pyridin-1-yl)cyclohexyl)thiazole-2- carboxamide |
| 356 | 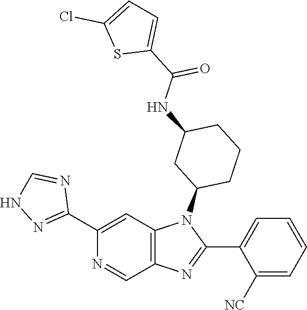 [SMILES: N#Cc1ccccc1-c1nc2cnc(-c3nc[nH]n3)cc2n1[C@@H]1CCC[C@H](NC(=O)c2ccc(Cl)s2)C1] | 5-chloro-N-((1S,3R)-3-(2-(2- cyanophenyl)-6-(1H-1,2,4-triazol-3- yl)-1H-imidazo[4,5-c]pyridin-1- yl)cyclohexyl)thiophene-2- carboxamide |
| 357 | 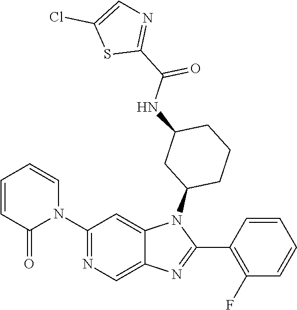 [SMILES: O=C(N[C@H]1CCC[C@@H](n2c(-c3ccccc3F)nc3cnc(-n4ccccc4=O)cc32)C1)c1ncc(Cl)s1] | 5-chloro-N-((1S,3R)-3-(2-(2- fluorophenyl)-6-(2-oxopyridin-1(2H)- yl)-1H-imidazo[4,5-c]pyridin-1- yl)cyclohexyl)thiazole-2-carboxamide |
| 358 | 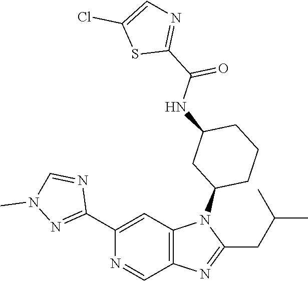 [SMILES: CC(C)Cc1nc2cnc(-c3ncn(C)n3)cc2n1[C@@H]1CCC[C@H](NC(=O)c2ncc(Cl)s2)C1] | 5-chloro-N-((1S,3R)-3-(2-isobutyl-6- (1-methyl-1H-1,2,4-triazol-3-yl)-1H- imidazo[4,5-c]pyridin-1- yl)cyclohexyl)thiazole-2-carboxamide |
| 359 | 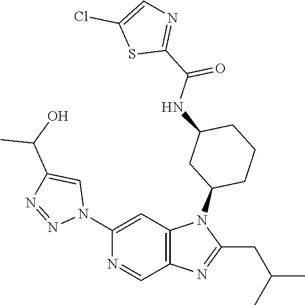 [SMILES: CC(C)Cc1nc2cnc(-n3cc(C(C)O)nn3)cc2n1[C@@H]1CCC[C@H](NC(=O)c2ncc(Cl)s2)C1] | 5-chloro-N-((1S,3R)-3-(6-(4-(1- hydroxyethyl)-1H-1,2,3-triazol-1-yl)- 2-isobutyl-1H-imidazo[4,5-c]pyridin- 1-yl)cyclohexyl)thiazole-2- carboxamide |
| 360 | 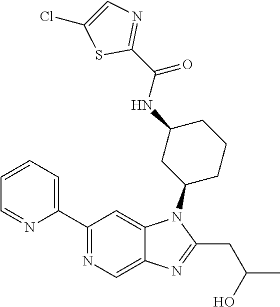 [SMILES: CC(O)Cc1nc2cnc(-c3ccccn3)cc2n1[C@@H]1CCC[C@H](NC(=O)c2ncc(Cl)s2)C1] | 5-chloro-N-((1S,3R)-3-(2-(2- hydroxypropyl)-6-(pyridin-2-yl)-1H- imidazo[4,5-c]pyridin-1- yl)cyclohexyl)thiazole-2-carboxamide |
| 361 | 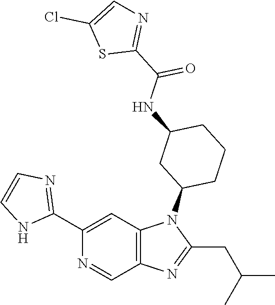 [SMILES: CC(C)Cc1nc2cnc(-c3ncc[nH]3)cc2n1[C@@H]1CCC[C@H](NC(=O)c2ncc(Cl)s2)C1] | N-((1S,3R)-3-(6-(1H-imidazol-2-yl)- 2-isobutyl-1H-imidazo[4,5-c]pyridin- 1-yl)cyclohexyl)-5-chlorothiazole-2- carboxamide |
| 362 | 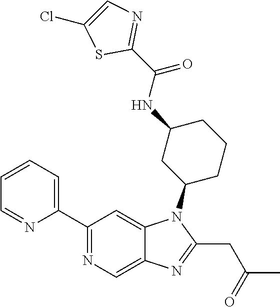 [SMILES: CC(=O)Cc1nc2cnc(-c3ccccn3)cc2n1[C@@H]1CCC[C@H](NC(=O)c2ncc(Cl)s2)C1] | 5-chloro-N-((1S,3R)-3-(2-(2- oxopropyl)-6-(pyridin-2-yl)-1H- imidazo[4,5-c]pyridin-1- yl)cyclohexyl)thiazole-2-carboxamide |
| 363 | 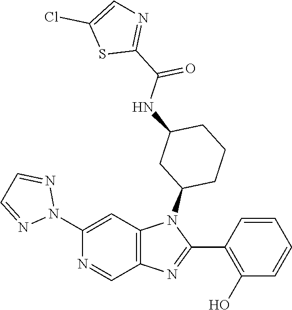 [SMILES: O=C(N[C@H]1CCC[C@@H](n2c(-c3ccccc3O)nc3cnc(-n4nccn4)cc32)C1)c1ncc(Cl)s1] | 5-chloro-N-((1S,3R)-3-(2-(2- hydroxyphenyl)-6-(2H-1,2,3-triazol- 2-yl)-1H-imidazo[4,5-c]pyridin-1- yl)cyclohexyl)thiazole-2-carboxamide |
| 364 | 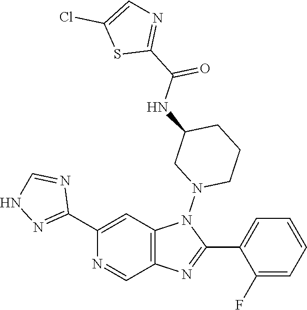 [SMILES: O=C(N[C@H]1CCCN(n2c(-c3ccccc3F)nc3cnc(-c4nc[nH]n4)cc32)C1)c1ncc(Cl)s1] | (S)-5-chloro-N-(1-(2-(2- fluorophenyl)-6-(1H-1,2,4-triazol-3- yl)-1H-imidazo[4,5-c]pyridin-1- yl)piperidin-3-yl)thiazole-2- carboxamide |
| 365 | 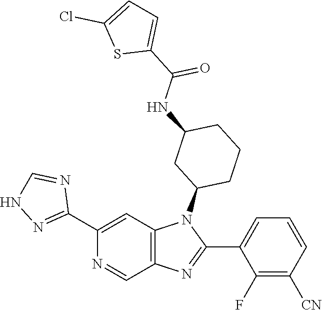 [SMILES: N#Cc1cccc(-c2nc3cnc(-c4nc[nH]n4)cc3n2[C@@H]2CCC[C@H](NC(=O)c3ccc(Cl)s3)C2)c1F] | 5-chloro-N-((1S,3R)-3-(2-(3-cyano-2- fluorophenyl)-6-(1H-1,2,4-triazol-3- yl)-1H-imidazo[4,5-c]pyridin-1- yl)cyclohexyl)thiophene-2- carboxamide |
| 366 | 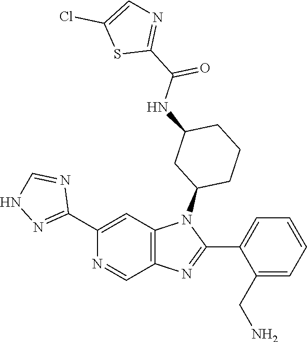 [SMILES: NCc1ccccc1-c1nc2cnc(-c3nc[nH]n3)cc2n1[C@@H]1CCC[C@H](NC(=O)c2ncc(Cl)s2)C1] | N-((1S,3R)-3-(2-(2- (aminomethyl)phenyl)-6-(1H-1,2,4- triazol-3-yl)-1H-imidazo[4,5- c]pyridin-1-yl)cyclohexyl)-5- chlorothiazole-2-carboxamide |
| 367 | 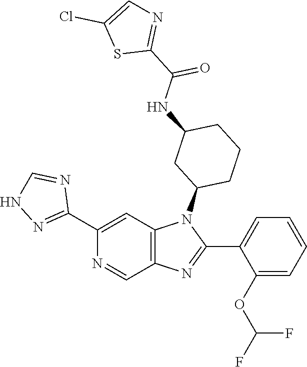 [SMILES: O=C(N[C@H]1CCC[C@@H](n2c(-c3ccccc3OC(F)F)nc3cnc(-c4nc[nH]n4)cc32)C1)c1ncc(Cl)s1] | 5-chloro-N-((1S,3R)-3-(2-(2- (difluoromethoxy)phenyl)-6-(1H- 1,2,4-triazol-3-yl)-1H-imidazo[4,5- c]pyridin-1-yl)cyclohexyl)thiazole-2- carboxamide |
| 368 | 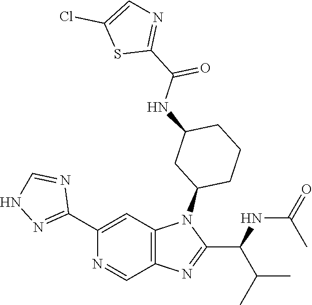 [SMILES: CC(=O)N[C@H](c1nc2cnc(-c3nc[nH]n3)cc2n1[C@@H]1CCC[C@H](NC(=O)c2ncc(Cl)s2)C1)C(C)C] | N-((1S,3R)-3-(2-((S)-1-acetamido-2- methylpropyl)-6-(1H-1,2,4-triazol-3- yl)-1H-imidazo[4,5-c]pyridin-1- yl)cyclohexyl)-5-chlorothiazole-2- carboxamide |
| 369 | 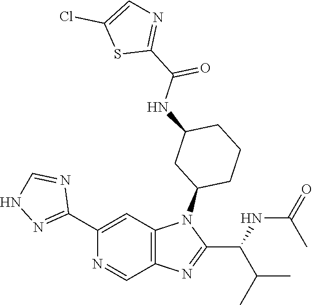 [SMILES: CC(=O)N[C@@H](c1nc2cnc(-c3nc[nH]n3)cc2n1[C@@H]1CCC[C@H](NC(=O)c2ncc(Cl)s2)C1)C(C)C] | N-((1S,3R)-3-(2-((R)-1-acetamido-2- methylpropyl)-6-(1H-1,2,4-triazol-3- yl)-1H-imidazo[4,5-c]pyridin-1- yl)cyclohexyl)-5-chlorothiazole-2- carboxamide |
| 370 | 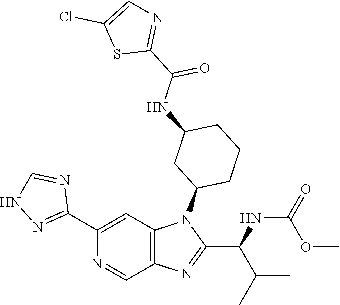 [SMILES: COC(=O)N[C@H](c1nc2cnc(-c3nc[nH]n3)cc2n1[C@@H]1CCC[C@H](NC(=O)c2ncc(Cl)s2)C1)C(C)C] | methyl ((S)-1-(1-((1R,3S)-3-(5- chlorothiazole-2- carboxamido)cyclohexyl)-6-(1H- 1,2,4-triazol-3-yl)-1H-imidazo[4,5- c]pyridin-2-yl)-2- methylpropyl)carbamate |
| 371 | 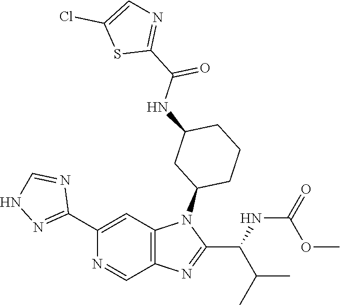 [SMILES: COC(=O)N[C@@H](c1nc2cnc(-c3nc[nH]n3)cc2n1[C@@H]1CCC[C@H](NC(=O)c2ncc(Cl)s2)C1)C(C)C] | methyl ((R)-1-(1-((1R,3S)-3-(5- chlorothiazole-2- carboxamido)cyclohexyl)-6-(1H- 1,2,4-triazol-3-yl)-1H-imidazo[4,5- c]pyridin-2-yl)-2- methylpropyl)carbamate |
| 372 | 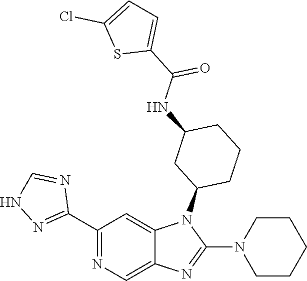 [SMILES: O=C(N[C@H]1CCC[C@@H](n2c(N3CCCCC3)nc3cnc(-c4nc[nH]n4)cc32)C1)c1ccc(Cl)s1] | 5-chloro-N-((1S,3R)-3-(2-(piperidin- 1-yl)-6-(1H-1,2,4-triazol-3-yl)-1H- imidazo[4,5-c]pyridin-1- yl)cyclohexyl)thiophene-2- carboxamide |
| 373 | 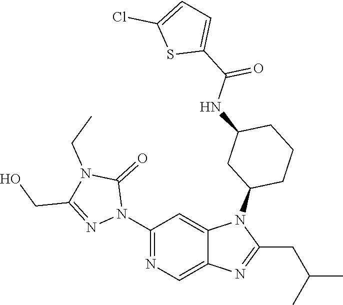 [SMILES: CCn1c(CO)nn(-c2cc3c(cn2)nc(CC(C)C)n3[C@@H]2CCC[C@H](NC(=O)c3ccc(Cl)s3)C2)c1=O] | 5-chloro-N-((1S,3R)-3-(6-(4-ethyl-3- (hydroxymethyl)-5-oxo-4,5-dihydro- 1H-1,2,4-triazol-1-yl)-2-isobutyl-1H- imidazo[4,5-c]pyridin-1- yl)cyclohexyl)thiophene-2- carboxamide |
| 374 | 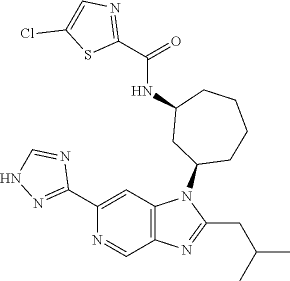 [SMILES: CC(C)Cc1nc2cnc(-c3nc[nH]n3)cc2n1[C@@H]1CCCC[C@H](NC(=O)c2ncc(Cl)s2)C1] | 5-chloro-N-((1S,3R)-3-(2-isobutyl-6- (1H-1,2,4-triazol-3-yl)-1H- imidazo[4,5-c]pyridin-1- yl)cycloheptyl)thiazole-2- carboxamide |
| 375 | 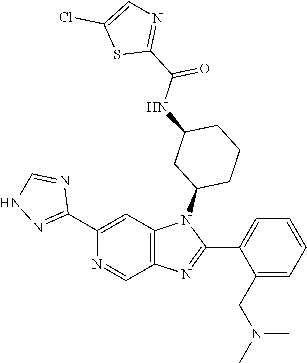 [SMILES: CN(C)Cc1ccccc1-c1nc2cnc(-c3nc[nH]n3)cc2n1[C@@H]1CCC[C@H](NC(=O)c2ncc(Cl)s2)C1] | 5-chloro-N-((1S,3R)-3-(2-(2- ((dimethylamino)methyl)phenyl)-6- (1H-1,2,4-triazol-3-yl)-1H- imidazo[4,5-c]pyridin-1- yl)cyclohexyl)thiazole-2-carboxamide |
| 376 | 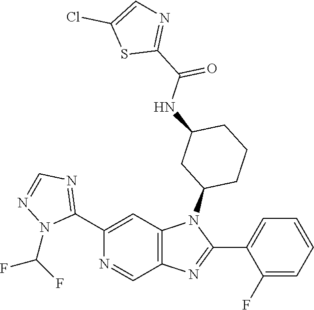 [SMILES: O=C(N[C@H]1CCC[C@@H](n2c(-c3ccccc3F)nc3cnc(-c4ncnn4C(F)F)cc32)C1)c1ncc(Cl)s1] | 5-chloro-N-((1S,3R)-3-(6-(1- (difluoromethyl)-1H-1,2,4-triazol-5- yl)-2-(2-fluorophenyl)-1H- imidazo[4,5-c]pyridin-1- yl)cyclohexyl)thiazole-2-carboxamide |
| 377 | 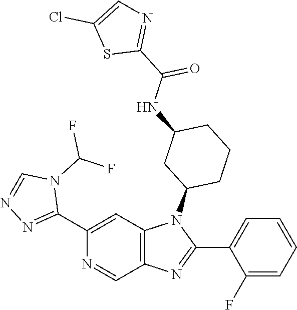 [SMILES: O=C(N[C@H]1CCC[C@@H](n2c(-c3ccccc3F)nc3cnc(-c4nncn4C(F)F)cc32)C1)c1ncc(Cl)s1] | 5-chloro-N-((1S,3R)-3-(6-(4- (difluoromethyl)-4H-1,2,4-triazol-3- yl)-2-(2-fluorophenyl)-1H- imidazo[4,5-c]pyridin-1- yl)cyclohexyl)thiazole-2-carboxamide |
| 378 | 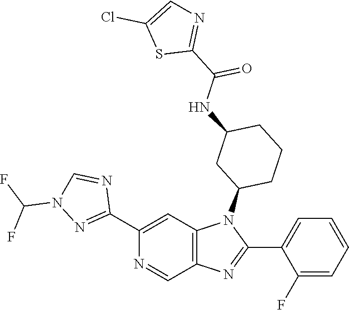 [SMILES: O=C(N[C@H]1CCC[C@@H](n2c(-c3ccccc3F)nc3cnc(-c4ncn(C(F)F)n4)cc32)C1)c1ncc(Cl)s1] | 5-chloro-N-((1S,3R)-3-(6-(1- (difluoromethyl)-1H-1,2,4-triazol-3- yl)-2-(2-fluorophenyl)-1H- imidazo[4,5-c]pyridin-1- yl)cyclohexyl)thiazole-2-carboxamide |
| 379 | 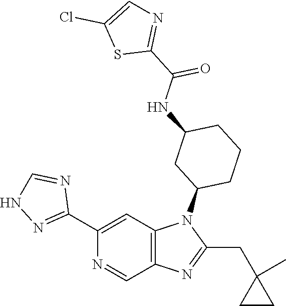 [SMILES: CC1(Cc2nc3cnc(-c4nc[nH]n4)cc3n2[C@@H]2CCC[C@H](NC(=O)c3ncc(Cl)s3)C2)CC1] | 5-chloro-N-((1S,3R)-3-(2-((1- methylcyclopropyl)methyl)-6-(1H- 1,2,4-triazol-3-yl)-1H-imidazo[4,5- c]pyridin-1-yl)cyclohexyl)thiazole-2- carboxamide |
| 380 | 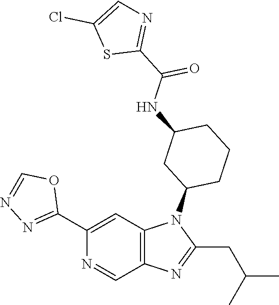 [SMILES: CC(C)Cc1nc2cnc(-c3nnco3)cc2n1[C@@H]1CCC[C@H](NC(=O)c2ncc(Cl)s2)C1] | 5-chloro-N-((1S,3R)-3-(2-isobutyl-6- (1,3,4-oxadiazol-2-yl)-1H- imidazo[4,5-c]pyridin-1- yl)cyclohexyl)thiazole-2-carboxamide |
| 381 | 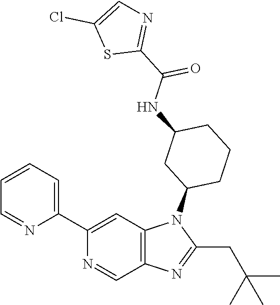 [SMILES: CC(C)(C)Cc1nc2cnc(-c3ccccn3)cc2n1[C@@H]1CCC[C@H](NC(=O)c2ncc(Cl)s2)C1] | 5-chloro-N-((1S,3R)-3-(2-neopentyl- 6-(pyridin-2-yl)-1H-imidazo[4,5- c]pyridin-1-yl)cyclohexyl)thiazole-2- carboxamide |
| 382 | 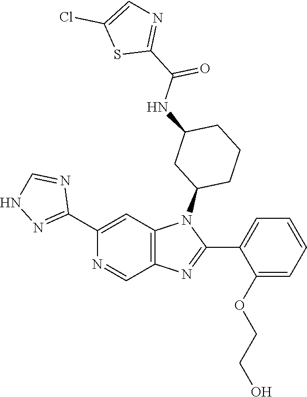 [SMILES: O=C(N[C@H]1CCC[C@@H](n2c(-c3ccccc3OCCO)nc3cnc(-c4nc[nH]n4)cc32)C1)c1ncc(Cl)s1] | 5-chloro-N-((1S,3R)-3-(2-(2-(2- hydroxyethoxy)phenyl)-6-(1H-1,2,4- triazol-3-yl)-1H-imidazo[4,5- c]pyridin-1-yl)cyclohexyl)thiazole-2- carboxamide |
| 383 | 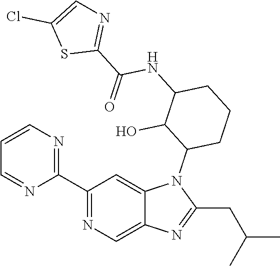 [SMILES: CC(C)Cc1nc2cnc(-c3ncccn3)cc2n1C1CCCC(NC(=O)c2ncc(Cl)s2)C1O] | 5-chloro-N-(2-hydroxy-3-(2-isobutyl- 6-(pyrimidin-2-yl)-1H-imidazo[4,5- c]pyridin-1-yl)cyclohexyl)thiazole-2- carboxamide(8) |
| 384 | 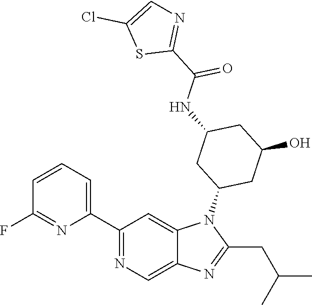 [SMILES: CC(C)Cc1nc2cnc(-c3cccc(F)n3)cc2n1[C@H]1C[C@H](O)C[C@@H](NC(=O)c2ncc(Cl)s2)C1] | 5-chloro-N-((1R,3S,5S)-3-(6-(6- fluoropyridin-2-yl)-2-isobutyl-1H- imidazo[4,5-c]pyridin-1-yl)-5- hydroxycyclohexyl)thiazole-2- carboxamide |
| 385 | 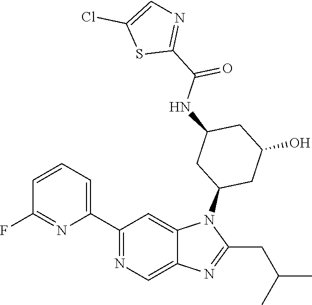 [SMILES: CC(C)Cc1nc2cnc(-c3cccc(F)n3)cc2n1[C@@H]1C[C@@H](O)C[C@H](NC(=O)c2ncc(Cl)s2)C1] | 5-chloro-N-((1S,3R,5R)-3-(6-(6- fluoropyridin-2-yl)-2-isobutyl-1H- imidazo[4,5-c]pyridin-1-yl)-5- hydroxycyclohexyl)thiazole-2- carboxamide |
| 386 | 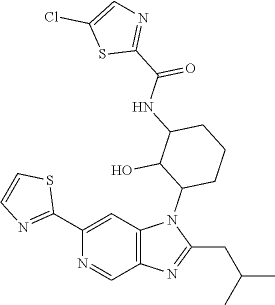 [SMILES: CC(C)Cc1nc2cnc(-c3nccs3)cc2n1C1CCCC(NC(=O)c2ncc(Cl)s2)C1O] | 5-chloro-N-(2-hydroxy-3-(2-isobutyl- 6-(thiazol-2-yl)-1H-imidazo[4,5- c]pyridin-1-yl)cyclohexyl)thiazole-2- carboxamide(8) |
| 387 | 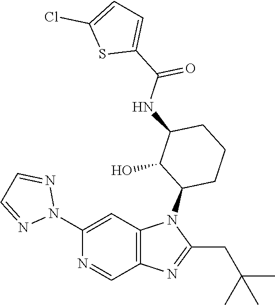 [SMILES: CC(C)(C)Cc1nc2cnc(-n3nccn3)cc2n1[C@@H]1CCC[C@H](NC(=O)c2ccc(Cl)s2)[C@H]1O] | 5-chloro-N-((1S,2R,3R)-2-hydroxy- 3-(2-neopentyl-6-(2H-1,2,3-triazol-2- yl)-1H-imidazo[4,5-c]pyridin-1- yl)cyclohexyl)thiophene-2- carboxamide |
| 388 | 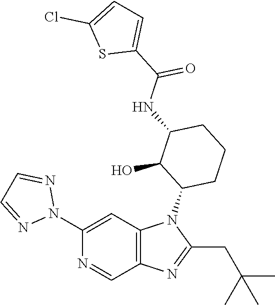 [SMILES: CC(C)(C)Cc1nc2cnc(-n3nccn3)cc2n1[C@H]1CCC[C@@H](NC(=O)c2ccc(Cl)s2)[C@@H]1O] | 5-chloro-N-((1R,2S,3S)-2-hydroxy-3- (2-neopentyl-6-(2H-1,2,3-triazol-2- yl)-1H-imidazo[4,5-c]pyridin-1- yl)cyclohexyl)thiophene-2- carboxamide |
| 389 | 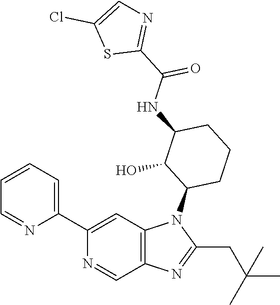 [SMILES: CC(C)(C)Cc1nc2cnc(-c3ccccn3)cc2n1[C@@H]1CCC[C@H](NC(=O)c2ncc(Cl)s2)[C@H]1O] | 5-chloro-N-((1S,2R,3R)-2-hydroxy- 3-(2-neopentyl-6-(pyridin-2-yl)-1H- imidazo[4,5-c]pyridin-1- yl)cyclohexyl)thiazole-2-carboxamide |
| 390 | 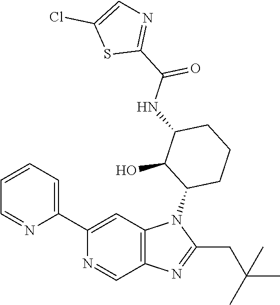 [SMILES: CC(C)(C)Cc1nc2cnc(-c3ccccn3)cc2n1[C@H]1CCC[C@@H](NC(=O)c2ncc(Cl)s2)[C@@H]1O] | 5-chloro-N-((1R,2S,3S)-2-hydroxy-3- (2-neopentyl-6-(pyridin-2-yl)-1H- imidazo[4,5-c]pyridin-1- yl)cyclohexyl)thiazole-2-carboxamide |
| 391 | 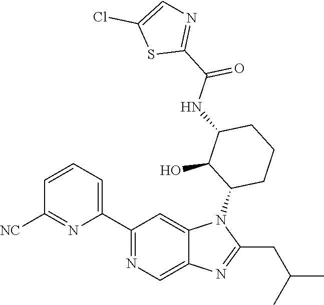 [SMILES: CC(C)Cc1nc2cnc(-c3cccc(C#N)n3)cc2n1[C@H]1CCC[C@@H](NC(=O)c2ncc(Cl)s2)[C@@H]1O] | 5-chloro-N-((1R,2S,3S)-3-(6-(6- cyanopyridin-2-yl)-2-isobutyl-1H- imidazo[4,5-c]pyridin-1-yl)-2- hydroxycyclohexyl)thiazole-2- carboxamide |
| 392 | 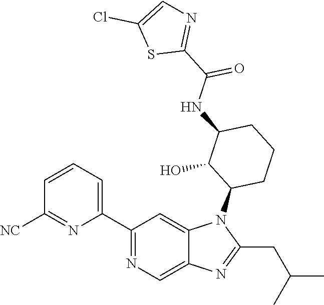 [SMILES: CC(C)Cc1nc2cnc(-c3cccc(C#N)n3)cc2n1[C@@H]1CCC[C@H](NC(=O)c2ncc(Cl)s2)[C@H]1O] | 5-chloro-N-((1S,2R,3R)-3-(6-(6- cyanopyridin-2-yl)-2-isobutyl-1H- imidazo[4,5-c]pyridin-1-yl)-2- hydroxycyclohexyl)thiazole-2- carboxamide |
| 393 | 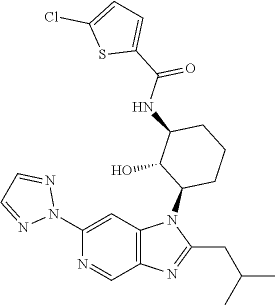 [SMILES: CC(C)Cc1nc2cnc(-n3nccn3)cc2n1[C@@H]1CCC[C@H](NC(=O)c2ccc(Cl)s2)[C@H]1O] | 5-chloro-N-((1S,2R,3R)-2-hydroxy- 3-(2-isobutyl-6-(2H-1,2,3-triazol-2- yl)-1H-imidazo[4,5-c]pyridin-1- yl)cyclohexyl)thiophene-2- carboxamide |
| 394 | 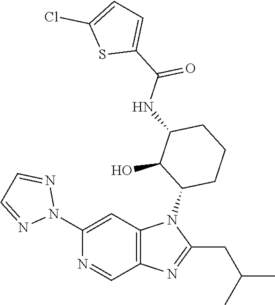 [SMILES: CC(C)Cc1nc2cnc(-n3nccn3)cc2n1[C@H]1CCC[C@@H](NC(=O)c2ccc(Cl)s2)[C@@H]1O] | 5-chloro-N-((1R,2S,3S)-2-hydroxy-3- (2-isobutyl-6-(2H-1,2,3-triazol-2-yl)- 1H-imidazo[4,5-c]pyridin-1- yl)cyclohexyl)thiophene-2- carboxamide |
| 395 | 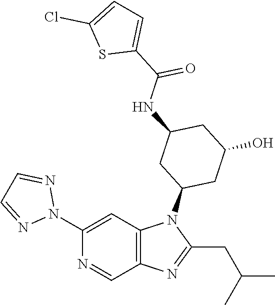 [SMILES: CC(C)Cc1nc2cnc(-n3nccn3)cc2n1[C@@H]1C[C@@H](O)C[C@H](NC(=O)c2ccc(Cl)s2)C1] | 5-chloro-N-((1R,3S,5S)-3-hydroxy-5- (2-isobutyl-6-(2H-1,2,3-triazol-2-yl)- 1H-imidazo[4,5-c]pyridin-1- yl)cyclohexyl)thiophene-2- carboxamide |
| 396 | 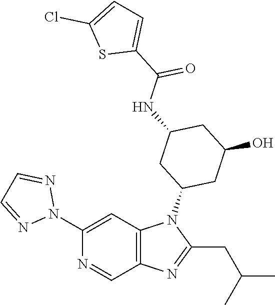 [SMILES: CC(C)Cc1nc2cnc(-n3nccn3)cc2n1[C@H]1C[C@H](O)C[C@@H](NC(=O)c2ccc(Cl)s2)C1] | 5-chloro-N-((1S,3R,5R)-3-hydroxy- 5-(2-isobutyl-6-(2H-1,2,3-triazol-2- yl)-1H-imidazo[4,5-c]pyridin-1- yl)cyclohexyl)thiophene-2- carboxamide |
| 397 | 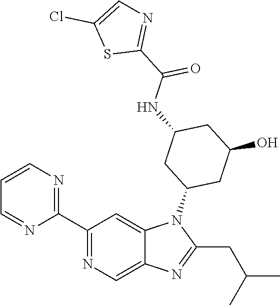 [SMILES: CC(C)Cc1nc2cnc(-c3ncccn3)cc2n1[C@H]1C[C@H](O)C[C@@H](NC(=O)c2ncc(Cl)s2)C1] | 5-chloro-N-((1R,3S,5S)-3-hydroxy-5- (2-isobutyl-6-(pyrimidin-2-yl)-1H- imidazo[4,5-c]pyridin-1- yl)cyclohexyl)thiazole-2-carboxamide |
| 398 | 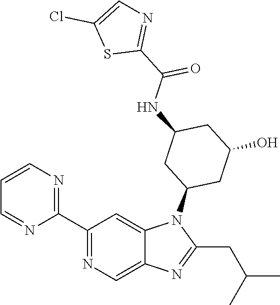 [SMILES: CC(C)Cc1nc2cnc(-c3ncccn3)cc2n1[C@@H]1C[C@@H](O)C[C@H](NC(=O)c2ncc(Cl)s2)C1] | 5-chloro-N-((1S,3R,5R)-3-hydroxy- 5-(2-isobutyl-6-(pyrimidin-2-yl)-1H- imidazo[4,5-c]pyridin-1- yl)cyclohexyl)thiazole-2-carboxamide |
| 399 | 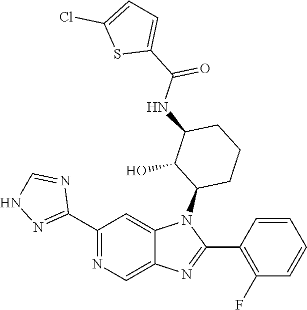 [SMILES: O=C(N[C@H]1CCC[C@@H](n2c(-c3ccccc3F)nc3cnc(-c4nc[nH]n4)cc32)[C@@H]1O)c1ccc(Cl)s1] | 5-chloro-N-((1S,2R,3R)-3-(2-(2- fluorophenyl)-6-(1H-1,2,4-triazol-3- yl)-1H-imidazo[4,5-c]pyridin-1-yl)-2- hydroxycyclohexyl)thiophene-2- carboxamide |
| 400 | 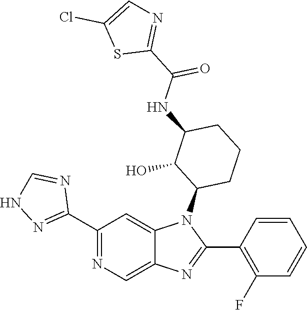 [SMILES: O=C(N[C@H]1CCC[C@@H](n2c(-c3ccccc3F)nc3cnc(-c4nc[nH]n4)cc32)[C@@H]1O)c1ncc(Cl)s1] | 5-chloro-N-((1S,2R,3R)-3-(2-(2- fluorophenyl)-6-(1H-1,2,4-triazol-3- yl)-1H-imidazo[4,5-c]pyridin-1-yl)-2- hydroxycyclohexyl)thiazole-2- carboxamide |
| 401 | 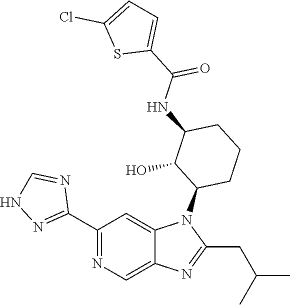 [SMILES: CC(C)Cc1nc2cnc(-c3nc[nH]n3)cc2n1[C@@H]1CCC[C@H](NC(=O)c2ccc(Cl)s2)[C@H]1O] | 5-chloro-N-((1S,2R,3R)-2-hydroxy- 3-(2-isobutyl-6-(1H-1,2,4-triazol-3- yl)-1H-imidazo[4,5-c]pyridin-1- yl)cyclohexyl)thiophene-2- carboxamide |
| 402 | 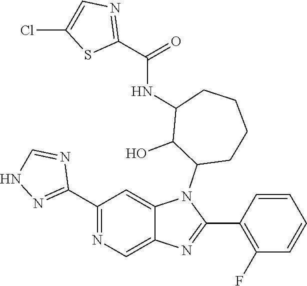 [SMILES: O=C(NC1CCCCC(n2c(-c3ccccc3F)nc3cnc(-c4nc[nH]n4)cc32)C1O)c1ncc(Cl)s1] | 5-chloro-N-((2-(2-fluorophenyl)-6- (1H-1,2,4-triazol-3-yl)-1H- imidazo[4,5-c]pyridin-1-yl)-2- hydroxycycloheptyl)thiazole-2- carboxamide(9) |
| 403 | 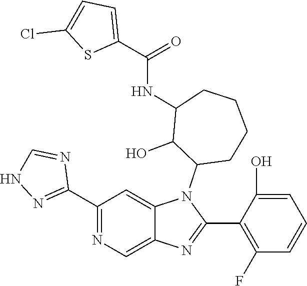 [SMILES: O=C(NC1CCCCC(n2c(-c3c(O)cccc3F)nc3cnc(-c4nc[nH]n4)cc32)C1O)c1ccc(Cl)s1] | 5-chloro-N-(3-(2-(2-fluoro-6- hydroxyphenyl)-6-(1H-1,2,4-triazol- 3-yl)-1H-imidazo[4,5-c]pyridin-1-yl)- 2-hydroxycycloheptyl)thiophene-2- carboxamide(9) |
| 404 | 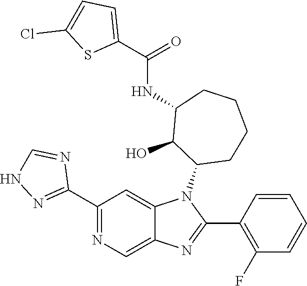 [SMILES: O=C(N[C@@H]1CCCC[C@H](n2c(-c3ccccc3F)nc3cnc(-c4nc[nH]n4)cc32)[C@H]1O)c1ccc(Cl)s1] | 5-chloro-N-((1R,2S,3S)-3-(2-(2- fluorophenyl)-6-(1H-1,2,4-triazol-3- yl)-1H-imidazo[4,5-c]pyridin-1-yl)-2- hydroxycycloheptyl)thiophene-2- carboxamide |
| 405 | 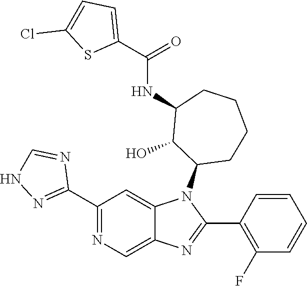 [SMILES: O=C(N[C@H]1CCCC[C@@H](n2c(-c3ccccc3F)nc3cnc(-c4nc[nH]n4)cc32)[C@@H]1O)c1ccc(Cl)s1] | 5-chloro-N-((1S,2R,3R)-3-(2-(2- fluorophenyl)-6-(1H-1,2,4-triazol-3- yl)-1H-imidazo[4,5-c]pyridin-1-yl)-2- hydroxycycloheptyl)thiophene-2- carboxamide |
| 406 | 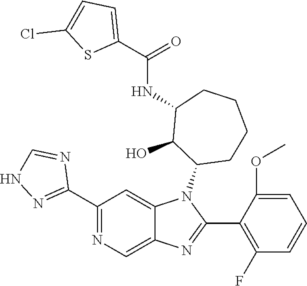 [SMILES: COc1cccc(F)c1-c1nc2cnc(-c3nc[nH]n3)cc2n1[C@H]1CCCC[C@@H](NC(=O)c2ccc(Cl)s2)[C@@H]1O] | 5-chloro-N-((1R,2S,3S)-3-(2-(2- fluoro-6-methoxyphenyl)-6-(1H- 1,2,4-triazol-3-yl)-1H-imidazo[4,5- c]pyridin-1-yl)-2- hydroxycycloheptyl)thiophene-2- carboxamide |
| 407 | 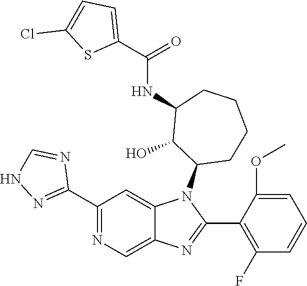 [SMILES: COc1cccc(F)c1-c1nc2cnc(-c3nc[nH]n3)cc2n1[C@@H]1CCCC[C@H](NC(=O)c2ccc(Cl)s2)[C@H]1O] | 5-chloro-N-((1S,2R,3R)-3-(2-(2- fluoro-6-methoxyphenyl)-6-(1H- 1,2,4-triazol-3-yl)-1H-imidazo[4,5- c]pyridin-1-yl)-2- hydroxycycloheptyl)thiophene-2- carboxamide |
| 408 | 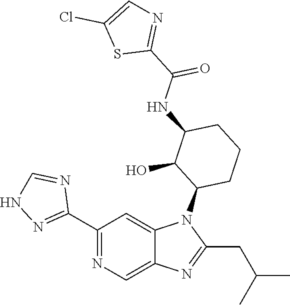 [SMILES: CC(C)Cc1nc2cnc(-c3nc[nH]n3)cc2n1[C@@H]1CCC[C@H](NC(=O)c2ncc(Cl)s2)[C@@H]1O] | 5-chloro-N-((1S,2S,3R)-2-hydroxy-3- (2-isobutyl-6-(1H-1,2,4-triazol-3-yl)- 1H-imidazo[4,5-c]pyridin-1- yl)cyclohexyl)thiazole-2-carboxamide |
| 409 | 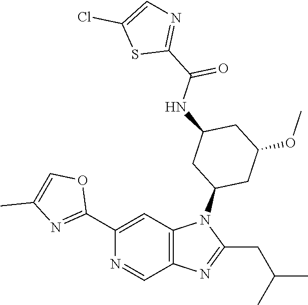 [SMILES: CO[C@H]1C[C@H](NC(=O)c2ncc(Cl)s2)C[C@H](n2c(CC(C)C)nc3cnc(-c4nc(C)co4)cc32)C1] | 5-chloro-N-((1R,3S,5S)-3-(2- isobutyl-6-(4-methyloxazol-2-yl)-1H- imidazo[4,5-c]pyridin-1-yl)-5- methoxycyclohexyl)thiazole-2- carboxamide |
| 410 | 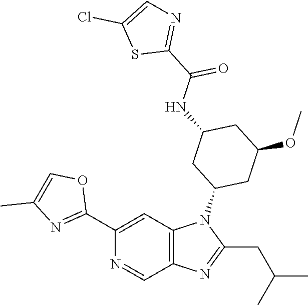 [SMILES: CO[C@@H]1C[C@@H](NC(=O)c2ncc(Cl)s2)C[C@@H](n2c(CC(C)C)nc3cnc(-c4nc(C)co4)cc32)C1] | 5-chloro-N-((1S,3R,5R)-3-(2- isobutyl-6-(4-methyloxazol-2-yl)-1H- imidazo[4,5-c]pyridin-1-yl)-5- methoxycyclohexyl)thiazole-2- carboxamide |
| 411 | 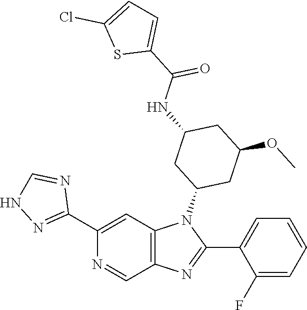 [SMILES: CO[C@@H]1C[C@@H](NC(=O)c2ccc(Cl)s2)C[C@@H](n2c(-c3ccccc3F)nc3cnc(-c4nc[nH]n4)cc32)C1] | 5-chloro-N-((1R,3S,5S)-3-(2-(2- fluorophenyl)-6-(1H-1,2,4-triazol-3- yl)-1H-imidazo[4,5-c]pyridin-1-yl)-5- methoxycyclohexyl)thiophene-2- carboxamide |
| 412 | 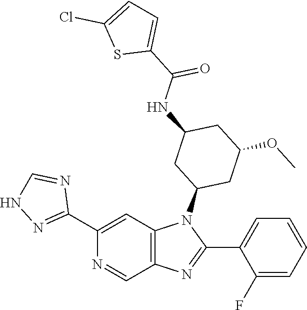 [SMILES: CO[C@H]1C[C@H](NC(=O)c2ccc(Cl)s2)C[C@H](n2c(-c3ccccc3F)nc3cnc(-c4nc[nH]n4)cc32)C1] | 5-chloro-N-((1S,3R,5R)-3-(2-(2- fluorophenyl)-6-(1H-1,2,4-triazol-3- yl)-1H-imidazo[4,5-c]pyridin-1-yl)-5- methoxycyclohexyl)thiophene-2- carboxamide |
| 413 | 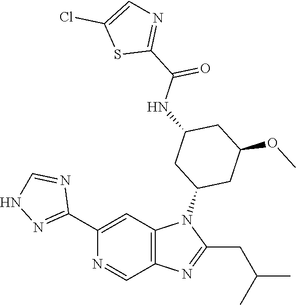 [SMILES: CO[C@@H]1C[C@@H](NC(=O)c2ncc(Cl)s2)C[C@@H](n2c(CC(C)C)nc3cnc(-c4nc[nH]n4)cc32)C1] | 5-chloro-N-((1R,3S,5S)-3-(2- isobutyl-6-(1H-1,2,4-triazol-3-yl)-1H- imidazo[4,5-c]pyridin-1-yl)-5- methoxycyclohexyl)thiazole-2- carboxamide |
| 414 | 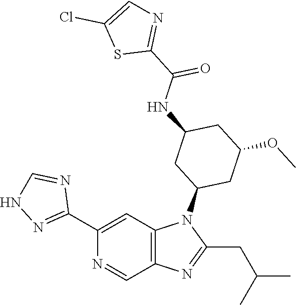 [SMILES: CO[C@H]1C[C@H](NC(=O)c2ncc(Cl)s2)C[C@H](n2c(CC(C)C)nc3cnc(-c4nc[nH]n4)cc32)C1] | 5-chloro-N-((1S,3R,5R)-3-(2- isobutyl-6-(1H-1,2,4-triazol-3-yl)-1H- imidazo[4,5-c]pyridin-1-yl)-5- methoxycyclohexyl)thiazole-2- carboxamide |
| 415 | 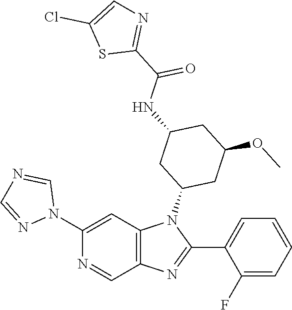 [SMILES: CO[C@@H]1C[C@@H](NC(=O)c2ncc(Cl)s2)C[C@@H](n2c(-c3ccccc3F)nc3cnc(-n4cncn4)cc32)C1] | 5-chloro-N-((1R,3S,5S)-3-(2-(2- fluorophenyl)-6-(1H-1,2,4-triazol-1- yl)-1H-imidazo[4,5-c]pyridin-1-yl)-5- methoxycyclohexyl)thiazole-2- carboxamide |
| 416 | 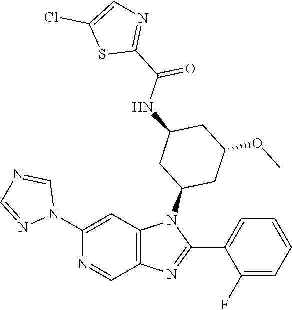 [SMILES: CO[C@H]1C[C@H](NC(=O)c2ncc(Cl)s2)C[C@H](n2c(-c3ccccc3F)nc3cnc(-n4cncn4)cc32)C1] | 5-chloro-N-((1S,3R,5R)-3-(2-(2- fluorophenyl)-6-(1H-1,2,4-triazol-1- yl)-1H-imidazo[4,5-c]pyridin-1-yl)-5- methoxycyclohexyl)thiazole-2- carboxamide |
| 417 | 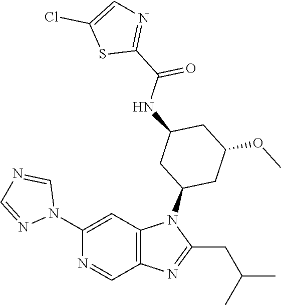 [SMILES: CO[C@H]1C[C@H](NC(=O)c2ncc(Cl)s2)C[C@H](n2c(CC(C)C)nc3cnc(-n4cncn4)cc32)C1] | 5-chloro-N-((1R,3S,5S)-3-(2- isobutyl-6-(1H-1,2,4-triazol-1-yl)-1H- imidazo[4,5-c]pyridin-1-yl)-5- methoxycyclohexyl)thiazole-2- carboxamide |
| 418 | 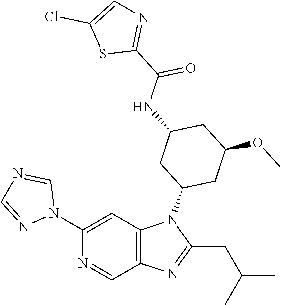 [SMILES: CO[C@@H]1C[C@@H](NC(=O)c2ncc(Cl)s2)C[C@@H](n2c(CC(C)C)nc3cnc(-n4cncn4)cc32)C1] | 5-chloro-N-((1S,3R,5R)-3-(2- isobutyl-6-(1H-1,2,4-triazol-1-yl)-1H- imidazo[4,5-c]pyridin-1-yl)-5- methoxycyclohexyl)thiazole-2- carboxamide |
| 419 | 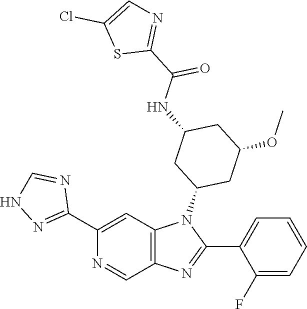 [SMILES: CO[C@H]1C[C@@H](NC(=O)c2ncc(Cl)s2)C[C@@H](n2c(-c3ccccc3F)nc3cnc(-c4nc[nH]n4)cc32)C1] | 5-chloro-N-((1R,3S,5R)-3-(2-(2- fluorophenyl)-6-(1H-1,2,4-triazol-3- yl)-1H-imidazo[4,5-c]pyridin-1-yl)-5- methoxycyclohexyl)thiazole-2- carboxamide |
| 420 | 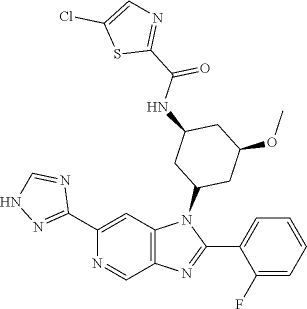 | 5-chloro-N-((1S,3R,5S)-3-(2-(2- fluorophenyl)-6-(1H-1,2,4-triazol-3- yl)-1H-imidazo[4,5-c]pyridin-1-yl)-5- methoxycyclohexyl)thiazole-2- carboxamide |
| 421 | 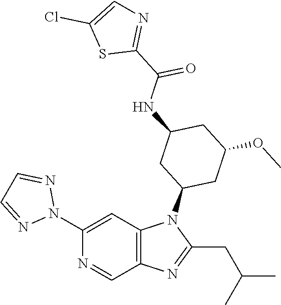 [SMILES: CO[C@H]1C[C@H](NC(=O)c2ncc(Cl)s2)C[C@H](n2c(CC(C)C)nc3cnc(-n4nccn4)cc32)C1] | 5-chloro-N-((1R,3S,5S)-3-(2- isobutyl-6-(2H-1,2,3-triazol-2-yl)-1H- imidazo[4,5-c]pyridin-1-yl)-5- methoxycyclohexyl)thiazole-2- carboxamide |
| 422 | 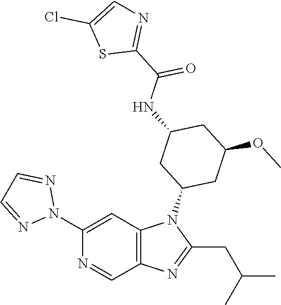 [SMILES: CO[C@@H]1C[C@@H](NC(=O)c2ncc(Cl)s2)C[C@@H](n2c(CC(C)C)nc3cnc(-n4nccn4)cc32)C1] | 5-chloro-N-((1S,3R,5R)-3-(2- isobutyl-6-(2H-1,2,3-triazol-2-yl)-1H- imidazo[4,5-c]pyridin-1-yl)-5- methoxycyclohexyl)thiazole-2- carboxamide |
| 423 | 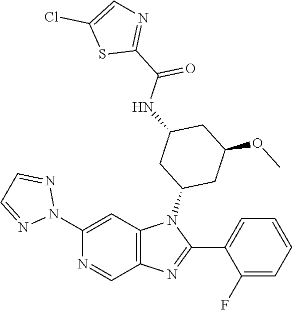 [SMILES: CO[C@@H]1C[C@@H](NC(=O)c2ncc(Cl)s2)C[C@@H](n2c(-c3ccccc3F)nc3cnc(-n4nccn4)cc32)C1] | 5-chloro-N-((1R,3S,5S)-3-(2-(2- fluorophenyl)-6-(2H-1,2,3-triazol-2- yl)-1H-imidazo[4,5-c]pyridin-1-yl)-5- methoxycyclohexyl)thiazole-2- carboxamide |
| 424 | 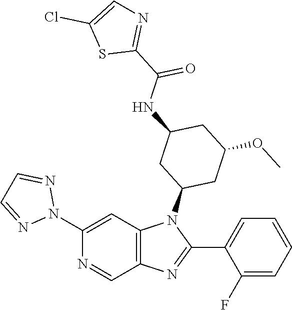 [SMILES: CO[C@H]1C[C@H](NC(=O)c2ncc(Cl)s2)C[C@H](n2c(-c3ccccc3F)nc3cnc(-n4nccn4)cc32)C1] | 5-chloro-N-((1S,3R,5R)-3-(2-(2- fluorophenyl)-6-(2H-1,2,3-triazol-2- yl)-1H-imidazo[4,5-c]pyridin-1-yl)-5- methoxycyclohexyl)thiazole-2- carboxamide |
| 425 | 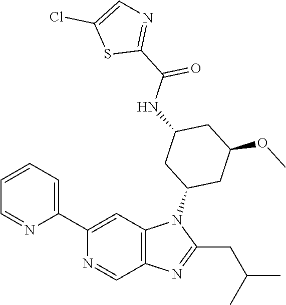 [SMILES: CO[C@@H]1C[C@@H](NC(=O)c2ncc(Cl)s2)C[C@@H](n2c(CC(C)C)nc3cnc(-c4ccccn4)cc32)C1] | 5-chloro-N-((1R,3S,5S)-3-(2- isobutyl-6-(pyridin-2-yl)-1H- imidazo[4,5-c]pyridin-1-yl)-5- methoxycyclohexyl)thiazole-2- carboxamide |
| 426 | 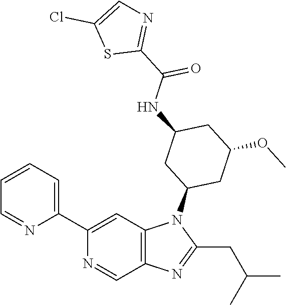 [SMILES: CO[C@H]1C[C@H](NC(=O)c2ncc(Cl)s2)C[C@H](n2c(CC(C)C)nc3cnc(-c4ccccn4)cc32)C1] | 5-chloro-N-((1S,3R,5R)-3-(2- isobutyl-6-(pyridin-2-yl)-1H- imidazo[4,5-c]pyridin-1-yl)-5- methoxycyclohexyl)thiazole-2- carboxamide |
| 427 | 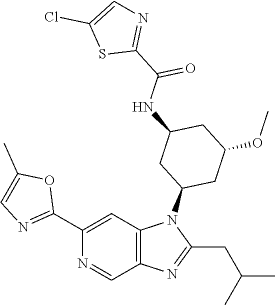 [SMILES: CO[C@H]1C[C@H](NC(=O)c2ncc(Cl)s2)C[C@H](n2c(CC(C)C)nc3cnc(-c4ncc(C)o4)cc32)C1] | 5-chloro-N-((1R,3S,5S)-3-(2- isobutyl-6-(5-methyloxazol-2-yl)-1H- imidazo[4,5-c]pyridin-1-yl)-5- methoxycyclohexyl)thiazole-2- carboxamide |
| 428 | 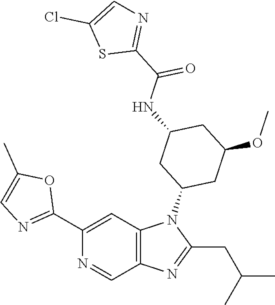 [SMILES: CO[C@@H]1C[C@@H](NC(=O)c2ncc(Cl)s2)C[C@@H](n2c(CC(C)C)nc3cnc(-c4ncc(C)o4)cc32)C1] | 5-chloro-N-((1S,3R,5R)-3-(2- isobutyl-6-(5-methyloxazol-2-yl)-1H- imidazo[4,5-c]pyridin-1-yl)-5- methoxycyclohexyl)thiazole-2- carboxamide |
| 429 | 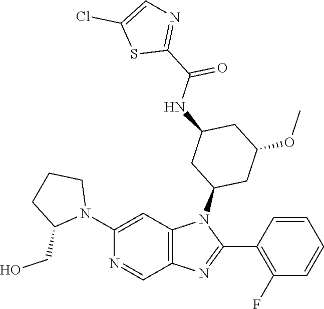 [SMILES: CO[C@H]1C[C@H](NC(=O)c2ncc(Cl)s2)C[C@H](n2c(-c3ccccc3F)nc3cnc(N4CCC[C@H]4CO)cc32)C1] | 5-chloro-N-((1R,3S,5S)-3-(2-(2- fluorophenyl)-6-((S)-2- (hydroxymethyl)pyrrolidin-1-yl)-1H- imidazo[4,5-c]pyridin-1-yl)-5- methoxycyclohexyl)thiazole-2- carboxamide |
| 430 | 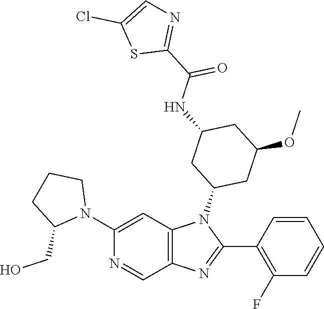 [SMILES: CO[C@@H]1C[C@@H](NC(=O)c2ncc(Cl)s2)C[C@@H](n2c(-c3ccccc3F)nc3cnc(N4CCC[C@H]4CO)cc32)C1] | 5-chloro-N-((1S,3R,5R)-3-(2-(2- fluorophenyl)-6-((S)-2- (hydroxymethyl)pyrrolidin-1-yl)-1H- imidazo[4,5-c]pyridin-1-yl)-5- methoxycyclohexyl)thiazole-2- carboxamide |
| 431 | 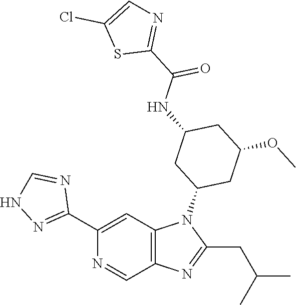 [SMILES: CO[C@H]1C[C@@H](NC(=O)c2ncc(Cl)s2)C[C@@H](n2c(CC(C)C)nc3cnc(-c4nc[nH]n4)cc32)C1] | 5-chloro-N-((1R,3S,5R)-3-(2- isobutyl-6-(1H-1,2,4-triazol-3-yl)-1H- imidazo[4,5-c]pyridin-1-yl)-5- methoxycyclohexyl)thiazole-2- carboxamide |
| 432 | 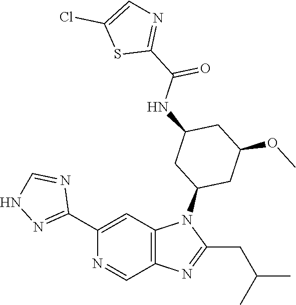 [SMILES: CO[C@@H]1C[C@H](NC(=O)c2ncc(Cl)s2)C[C@H](n2c(CC(C)C)nc3cnc(-c4nc[nH]n4)cc32)C1] | 5-chloro-N-((1S,3R,5S)-3-(2- isobutyl-6-(1H-1,2,4-triazol-3-yl)-1H- imidazo[4,5-c]pyridin-1-yl)-5- methoxycyclohexyl)thiazole-2- carboxamide |
| 433 | 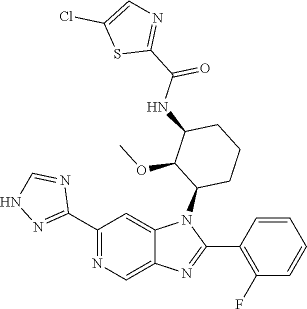 [SMILES: CO[C@H]1[C@@H](NC(=O)c2ncc(Cl)s2)CCC[C@H]1n1c(-c2ccccc2F)nc2cnc(-c3nc[nH]n3)cc21] | 5-chloro-N-((1S,2S,3R)-3-(2-(2- fluorophenyl)-6-(1H-1,2,4-triazol-3- yl)-1H-imidazo[4,5-c]pyridin-1-yl)-2- methoxycyclohexyl)thiazole-2- carboxamide |
| 434 | 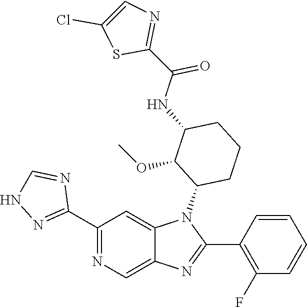 [SMILES: CO[C@@H]1[C@H](NC(=O)c2ncc(Cl)s2)CCC[C@@H]1n1c(-c2ccccc2F)nc2cnc(-c3nc[nH]n3)cc21] | 5-chloro-N-((1R,2R,3S)-3-(2-(2- fluorophenyl)-6-(1H-1,2,4-triazol-3- yl)-1H-imidazo[4,5-c]pyridin-1-yl)-2- methoxycyclohexyl)thiazole-2- carboxamide |
| 435 | 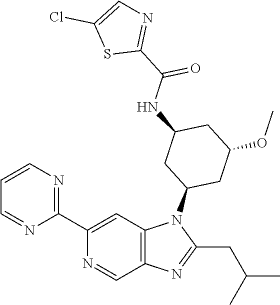 [SMILES: CO[C@H]1C[C@H](NC(=O)c2ncc(Cl)s2)C[C@H](n2c(CC(C)C)nc3cnc(-c4ncccn4)cc32)C1] | 5-chloro-N-((1R,3S,5S)-3-(2- isobutyl-6-(pyrimidin-2-yl)-1H- imidazo[4,5-c]pyridin-1-yl)-5- methoxycyclohexyl)thiazole-2- carboxamide |
| 436 | 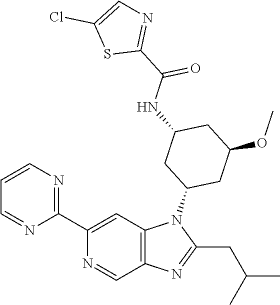 [SMILES: CO[C@@H]1C[C@@H](NC(=O)c2ncc(Cl)s2)C[C@@H](n2c(CC(C)C)nc3cnc(-c4ncccn4)cc32)C1] | 5-chloro-N-((1S,3R,5R)-3-(2- isobutyl-6-(pyrimidin-2-yl)-1H- imidazo[4,5-c]pyridin-1-yl)-5- methoxycyclohexyl)thiazole-2- carboxamide |
| 437 | 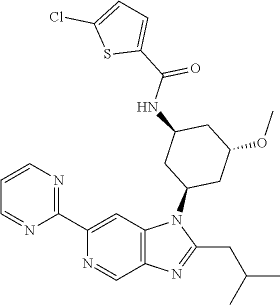 [SMILES: CO[C@H]1C[C@H](NC(=O)c2ccc(Cl)s2)C[C@H](n2c(CC(C)C)nc3cnc(-c4ncccn4)cc32)C1] | 5-chloro-N-((1R,3S,5S)-3-(2- isobutyl-6-(pyrimidin-2-yl)-1H- imidazo[4,5-c]pyridin-1-yl)-5- methoxycyclohexyl)thiophene-2- carboxamide |
| 438 | 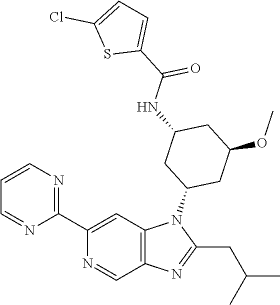 [SMILES: CO[C@@H]1C[C@@H](NC(=O)c2ccc(Cl)s2)C[C@@H](n2c(CC(C)C)nc3cnc(-c4ncccn4)cc32)C1] | 5-chloro-N-((1S,3R,5R)-3-(2- isobutyl-6-(pyrimidin-2-yl)-1H- imidazo[4,5-c]pyridin-1-yl)-5- methoxycyclohexyl)thiophene-2- carboxamide |
| 439 | 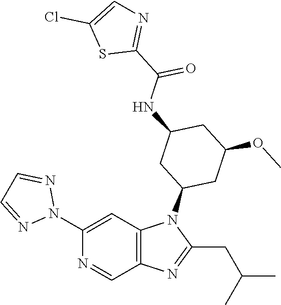 [SMILES: CO[C@@H]1C[C@H](NC(=O)c2ncc(Cl)s2)C[C@H](n2c(CC(C)C)nc3cnc(-n4nccn4)cc32)C1] | 5-chloro-N-((1R,3S,5R)-3-(2- isobutyl-6-(2H-1,2,3-triazol-2-yl)-1H- imidazo[4,5-c]pyridin-1-yl)-5- methoxycyclohexyl)thiazole-2- carboxamide |
| 440 | 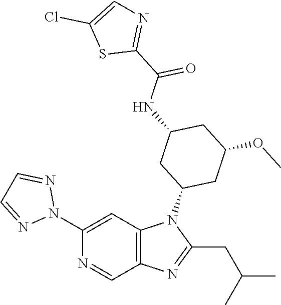 [SMILES: CO[C@H]1C[C@@H](NC(=O)c2ncc(Cl)s2)C[C@@H](n2c(CC(C)C)nc3cnc(-n4nccn4)cc32)C1] | 5-chloro-N-((1S,3R,5S)-3-(2- isobutyl-6-(2H-1,2,3-triazol-2-yl)-1H- imidazo[4,5-c]pyridin-1-yl)-5- methoxycyclohexyl)thiazole-2- carboxamide |
| 441 | 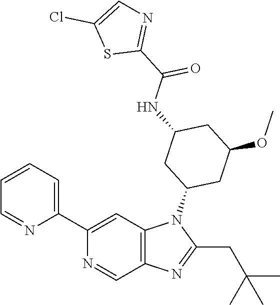 [SMILES: CO[C@@H]1C[C@@H](NC(=O)c2ncc(Cl)s2)C[C@@H](n2c(CC(C)(C)C)nc3cnc(-c4ccccn4)cc32)C1] | 5-chloro-N-((1S,3R,5R)-3-methoxy- 5-(2-neopentyl-6-(pyridin-2-yl)-1H- imidazo[4,5-c]pyridin-1- yl)cyclohexyl)thiazole-2-carboxamide |
| 442 | 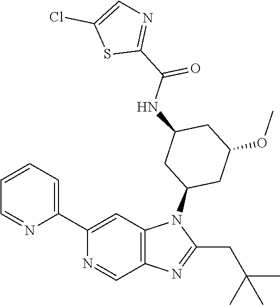 [SMILES: CO[C@H]1C[C@H](NC(=O)c2ncc(Cl)s2)C[C@H](n2c(CC(C)(C)C)nc3cnc(-c4ccccn4)cc32)C1] | 5-chloro-N-((1R,3S,5S)-3-methoxy- 5-(2-neopentyl-6-(pyridin-2-yl)-1H- imidazo[4,5-c]pyridin-1- yl)cyclohexyl)thiazole-2-carboxamide |
| 443 | 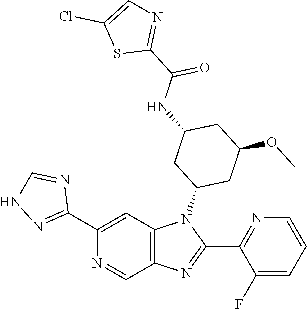 [SMILES: CO[C@@H]1C[C@@H](NC(=O)c2ncc(Cl)s2)C[C@@H](n2c(-c3ncccc3F)nc3cnc(-c4nc[nH]n4)cc32)C1] | 5-chloro-N-((1R,3S,5S)-3-(2-(3- fluoropyridin-2-yl)-6-(1H-1,2,4- triazol-3-yl)-1H-imidazo[4,5-c]- 175 - yridine-1-yl)-5- methoxycyclohexyl)thiazole-2- carboxamide |
| 444 | 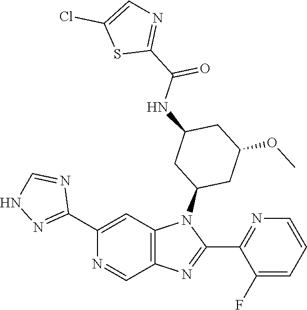 [SMILES: CO[C@H]1C[C@H](NC(=O)c2ncc(Cl)s2)C[C@H](n2c(-c3ncccc3F)nc3cnc(-c4nc[nH]n4)cc32)C1] | 5-chloro-N-((1S,3R,5R)-3-(2-(3- fluoropyridin-2-yl)-6-(1H-1,2,4- triazol-3-yl)-1H-imidazo[4,5- c]pyridin-1-yl)-5- methoxycyclohexyl)thiazole-2- carboxamide |
| 445 | 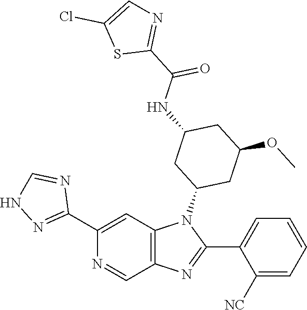 [SMILES: CO[C@@H]1C[C@@H](NC(=O)c2ncc(Cl)s2)C[C@@H](n2c(-c3ccccc3C#N)nc3cnc(-c4nc[nH]n4)cc32)C1] | 5-chloro-N-((1R,3S,5S)-3-(2-(2- cyanophenyl)-6-(1H-1,2,4-triazol-3- yl)-1H-imidazo[4,5-c]pyridin-1-yl)-5- methoxycyclohexyl)thiazole-2- carboxamide |
| 446 | 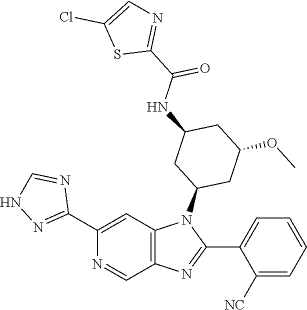 [SMILES: CO[C@H]1C[C@H](NC(=O)c2ncc(Cl)s2)C[C@H](n2c(-c3ccccc3C#N)nc3cnc(-c4nc[nH]n4)cc32)C1] | 5-chloro-N-((1S,3R,5R)-3-(2-(2- cyanophenyl)-6-(1H-1,2,4-triazol-3- yl)-1H-imidazo[4,5-c]pyridin-1-yl)-5- methoxycyclohexyl)thiazole-2- carboxamide |
| 447 | 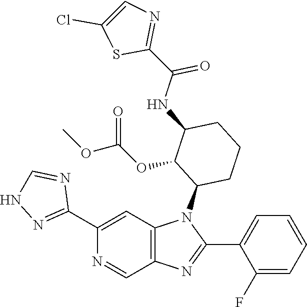 [SMILES: COC(=O)O[C@@H]1[C@@H](NC(=O)c2ncc(Cl)s2)CCC[C@H]1n1c(-c2ccccc2F)nc2cnc(-c3nc[nH]n3)cc21] | (1R,2S,6R)-2-(5-chlorothiazole-2- carboxamido)-6-(2-(2-fluorophenyl)- 6-(1H-1,2,4-triazol-3-yl)-1H- imidazo[4,5-c]pyridin-1- yl)cyclohexyl methyl carbonate |
| 448 | 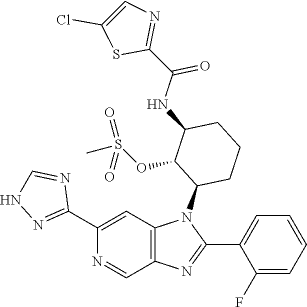 [SMILES: CS(=O)(=O)O[C@@H]1[C@@H](NC(=O)c2ncc(Cl)s2)CCC[C@H]1n1c(-c2ccccc2F)nc2cnc(-c3nc[nH]n3)cc21] | (1R,2S,6R)-2-(5-chlorothiazole-2- carboxamido)-6-(2-(2-fluorophenyl)- 6-(1H-1,2,4-triazol-3-yl)-1H- imidazo[4,5-c]pyridin-1- yl)cyclohexyl methanesulfonate |
| 449 | 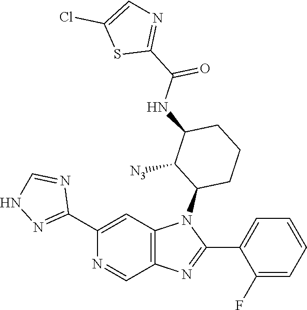 [SMILES: [N-]=[N+]=N[C@@H]1[C@@H](NC(=O)c2ncc(Cl)s2)CCC[C@H]1n1c(-c2ccccc2F)nc2cnc(-c3nc[nH]n3)cc21] | N-((1S,2R,3R)-2-azido-3-(2-(2- fluorophenyl)-6-(1H-1,2,4-triazol-3- yl)-1H-imidazo[4,5-c]pyridin-1- yl)cyclohexyl)-5-chlorothiazole-2- carboxamide |
| 450 | 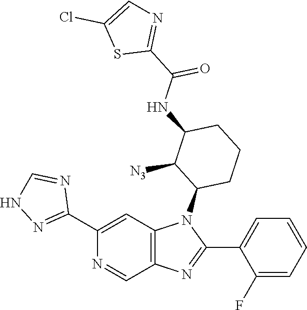 [SMILES: [N-]=[N+]=N[C@H]1[C@@H](NC(=O)c2ncc(Cl)s2)CCC[C@H]1n1c(-c2ccccc2F)nc2cnc(-c3nc[nH]n3)cc21] | N-((1S,2S,3R)-2-azido-3-(2-(2- fluorophenyl)-6-(1H-1,2,4-triazol-3- yl)-1H-imidazo[4,5-c]pyridin-1- yl)cyclohexyl)-5-chlorothiazole-2- carboxamide |
| 451 | 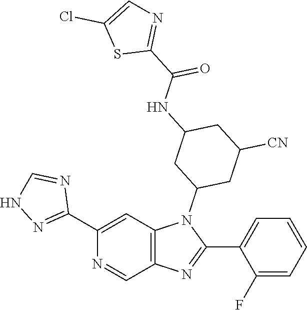 [SMILES: N#CC1CC(NC(=O)c2ncc(Cl)s2)CC(n2c(-c3ccccc3F)nc3cnc(-c4nc[nH]n4)cc32)C1] | 5-chloro-N-(3-cyano-5-(2-(2- fluorophenyl)-6-(1H-1,2,4-triazol-3- yl)-1H-imidazo[4,5-c]pyridin-1- yl)cyclohexyl)thiazole-2- carboxamide(11) |
| 452 | 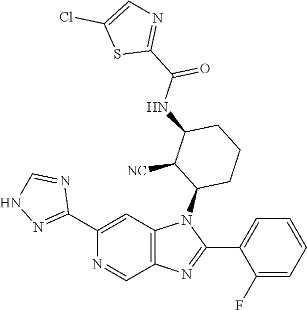 [SMILES: N#C[C@H]1[C@@H](NC(=O)c2ncc(Cl)s2)CCC[C@H]1n1c(-c2ccccc2F)nc2cnc(-c3nc[nH]n3)cc21] | 5-chloro-N-((1S,2S,3R)-2-cyano-3- (2-(2-fluorophenyl)-6-(1H-1,2,4- triazol-3-yl)-1H-imidazo[4,5- c]pyridin-1-yl)cyclohexyl)thiazole-2- carboxamide |
| 453 | 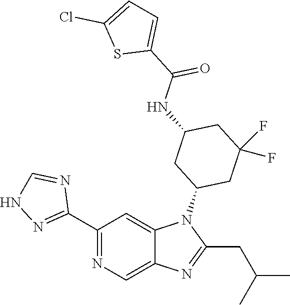 [SMILES: CC(C)Cc1nc2cnc(-c3nc[nH]n3)cc2n1[C@@H]1C[C@H](NC(=O)c2ccc(Cl)s2)CC(F)(F)C1] | 5-chloro-N-((1R,5S)-3,3-difluoro-5- (2-isobutyl-6-(1H-1,2,4-triazol-3-yl)- 1H-imidazo[4,5-c]pyridin-1- yl)cyclohexyl)thiophene-2- carboxamide |
| 454 | 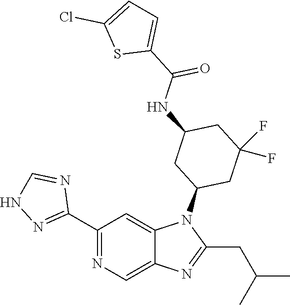 [SMILES: CC(C)Cc1nc2cnc(-c3nc[nH]n3)cc2n1[C@H]1C[C@@H](NC(=O)c2ccc(Cl)s2)CC(F)(F)C1] | 5-chloro-N-((1S,5R)-3,3-difluoro-5- (2-isobutyl-6-(1H-1,2,4-triazol-3-yl)- 1H-imidazo[4,5-c]pyridin-1- yl)cyclohexyl)thiophene-2- carboxamide |
| 455 | 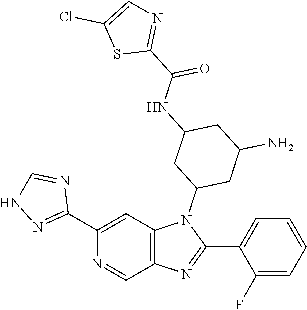 [SMILES: NC1CC(NC(=O)c2ncc(Cl)s2)CC(n2c(-c3ccccc3F)nc3cnc(-c4nc[nH]n4)cc32)C1] | N-(3-amino-5-(2-(2-fluorophenyl)-6- (1H-1,2,4-triazol-3-yl)-1H- imidazo[4,5-c]pyridin-1- yl)cyclohexyl)-5-chlorothiazole-2- carboxamide(11) |
| 456 | 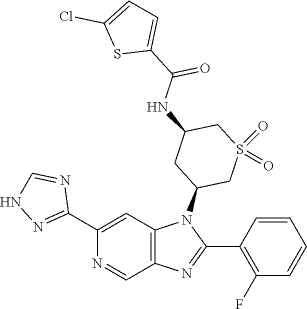 [SMILES: O=C(N[C@@H]1C[C@H](n2c(-c3ccccc3F)nc3cnc(-c4nc[nH]n4)cc32)CS(=O)(=O)C1)c1ccc(Cl)s1] | 5-chloro-N-((3R,5S)-5-(2-(2- fluorophenyl)-6-(1H-1,2,4-triazol-3- yl)-1H-imidazo[4,5-c]pyridin-1-yl)- 1,1-dioxidotetrahydro-2H-thiopyran- 3-yl)thiophene-2-carboxamide |
| 457 | 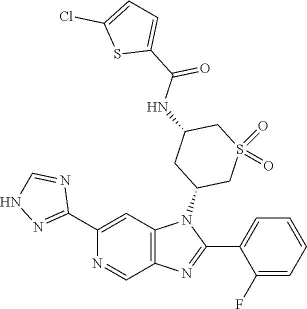 [SMILES: O=C(N[C@H]1C[C@@H](n2c(-c3ccccc3F)nc3cnc(-c4nc[nH]n4)cc32)CS(=O)(=O)C1)c1ccc(Cl)s1] | 5-chloro-N-((3S,5R)-5-(2-(2- fluorophenyl)-6-(1H-1,2,4-triazol-3- yl)-1H-imidazo[4,5-c]pyridin-1-yl)- 1,1-dioxidotetrahydro-2H-thiopyran- 3-yl)thiophene-2-carboxamide |
| 458 | 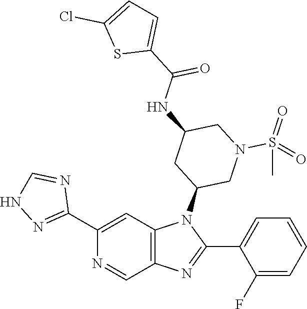 [SMILES: CS(=O)(=O)N1C[C@H](NC(=O)c2ccc(Cl)s2)C[C@H](n2c(-c3ccccc3F)nc3cnc(-c4nc[nH]n4)cc32)C1] | 5-chloro-N-((3R,5S)-5-(2-(2- fluorophenyl)-6-(1H-1,2,4-triazol-3- yl)-1H-imidazo[4,5-c]pyridin-1-yl)-1- (methylsulfonyl)piperidin-3- yl)thiophene-2-carboxamide |
| 459 | 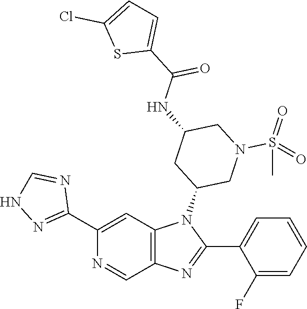 [SMILES: CS(=O)(=O)N1C[C@@H](NC(=O)c2ccc(Cl)s2)C[C@@H](n2c(-c3ccccc3F)nc3cnc(-c4nc[nH]n4)cc32)C1] | 5-chloro-N-((3S,5R)-5-(2-(2- fluorophenyl)-6-(1H-1,2,4-triazol-3- yl)-1H-imidazo[4,5-c]pyridin-1-yl)-1- (methylsulfonyl)piperidin-3- yl)thiophene-2-carboxamide |
| 460 | 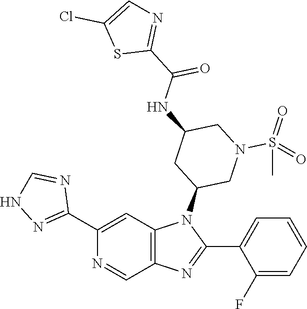 [SMILES: CS(=O)(=O)N1C[C@H](NC(=O)c2ncc(Cl)s2)C[C@H](n2c(-c3ccccc3F)nc3cnc(-c4nc[nH]n4)cc32)C1] | 5-chloro-N-((3R,5S)-5-(2-(2- fluorophenyl)-6-(1H-1,2,4-triazol-3- yl)-1H-imidazo[4,5-c]pyridin-1-yl)-1- (methylsulfonyl)piperidin-3- yl)thiazole-2-carboxamide |
| 461 | 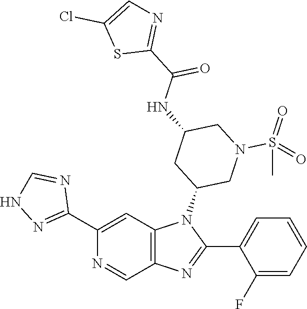 [SMILES: CS(=O)(=O)N1C[C@@H](NC(=O)c2ncc(Cl)s2)C[C@@H](n2c(-c3ccccc3F)nc3cnc(-c4nc[nH]n4)cc32)C1] | 5-chloro-N-((3S,5R)-5-(2-(2- fluorophenyl)-6-(1H-1,2,4-triazol-3- yl)-1H-imidazo[4,5-c]pyridin-1-yl)-1- (methylsulfonyl)piperidin-3- yl)thiazole-2-carboxamide |
| 462 | 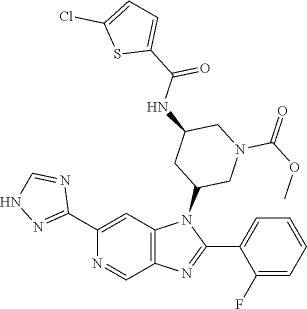 | methyl (3R,5S)-3-(5- chlorothiophene-2-carboxamido)-5- (2-(2-fluorophenyl)-6-(1H-1,2,4- triazol-3-yl)-1H-imidazo[4,5- c]pyridin-1-yl)piperidine-1- carboxylate |
| 463 | 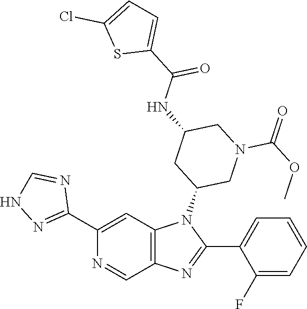 [SMILES: COC(=O)N1C[C@@H](NC(=O)c2ccc(Cl)s2)C[C@@H](n2c(-c3ccccc3F)nc3cnc(-c4nc[nH]n4)cc32)C1] | methyl (3S,5R)-3-(5- chlorothiophene-2-carboxamido)-5- (2-(2-fluorophenyl)-6-(1H-1,2,4- triazol-3-yl)-1H-imidazo[4,5- c]pyridin-1-yl)piperidine-1- carboxylate |
| 464 | 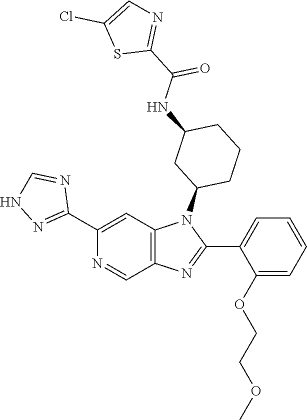 [SMILES: COCCOc1ccccc1-c1nc2cnc(-c3nc[nH]n3)cc2n1[C@@H]1CCC[C@H](NC(=O)c2ncc(Cl)s2)C1] | 5-chloro-N-((1S,3R)-3-(2-(2-(2- methoxyethoxy)phenyl)-6-(1H-1,2,4- triazol-3-yl)-1H-imidazo[4,5- c]pyridin-1-yl)cyclohexyl)thiazole-2- carboxamide |

⁽¹⁾ relative stereochemistry at centers 1 and 3 of cyclohexyl ring is cis.
⁽²⁾ relative stereochemistry at centers 1 and 3 of cycloheptyl ring is cis.
⁽³⁾ relative stereochemistry at centers 1 and 3 of cyclopentyl ring is cis.
⁽⁴⁾ relative stereochemistry at centers 3 and 5 of piperidine ring is cis.
⁽⁵⁾ relative stereochemistry at centers 3 and 5 of tetrahydropyranyl ring is cis.
⁽⁶⁾ relative stereochemistry at centers 3 and 5 of tetrahydropyranyl ring is trans.
⁽⁷⁾ relative stereochemistry at centers 1 and 3 of cyclohexyl ring is cis with -OH at center 5 trans.
⁽⁸⁾ relative stereochemistry at centers 1 and 3 of the cyclohexyl ring is cis with -OH at center 2 trans.
⁽¹⁰⁾ relative stereochemistry at centers 1 and 3 of the cycloheptyl ring is cis with -OH at center 2 trans.
⁽¹¹⁾ relative stereochemistry at centers 1 and 5 of the cyclohexyl ring is cis with stereochemistry unassigned at center 3.

In further embodiments, the compound of Formula IA is one or more of Examples 5-8, 11, 13-15, 16-20, 22-25, 31, 39, 53, 55, 61, 62, 65, 66, 68, 71, 72, 74-78, 80, 83-85, 86, 88, 104, 105, 108, 110, 113, 115, 116, 117, 118, 123, 128, 132, 134, 136, 141, 142, 162, 163, 165, 168-170, 175, 179, 181, 184-186, 189, 196, 202, 214, 218, 220, 227, 235, 238, 240, 242, 243, 245, 246, 248, 250, 252, 254, 256, 258, 260, 264, 312, 313, 317, 325, 326, 330, 331, 338-342, 344, 347, 348, 352, 354, 355, 356, 363, 364, 374, 378, 379, 386, 387, 389, 393, 395, 399-402, 405, 407, 412, 414, 416, 420, 424, 429, 432, 435, 437, 439, 444, 446, 458, 460, or 462, or a pharmaceutically acceptable salt thereof. In yet other embodiments, the compound of Formula IA is one or more of Examples 108, 110, 115, 214, 218, 252, 254, 256, 258, 260, 395, 398-401, 412, 414, 416, 420, 424, 429, 432, 435, 437, 439, 444, or 446, or a pharmaceutically acceptable salt thereof. In still other embodiments, the compound of Formula IA is one or more of Examples 113, 220, 227, 235, 238, 240, 242, 243, 245, 246, 248, 250, 386, 387, 389, 393, 402, 405, or 407, or a pharmaceutically acceptable salt thereof.

In further embodiments, the compound of Formula IB is one or more of Examples 151, 152, 155, or 157, or a pharmaceutically acceptable salt thereof.

In other embodiments, the following Formula IA compounds (including all stereoisomers), and their pharmaceutically acceptable salts, tautomers, and/or isotopologues, can be synthesized using the Intermediate and General Schemes and Examples set forth herein, or a combination of the procedures described herein using ordinary skill in the art.

TABLE 1A
5-chloro-N-(5-(2-(2-(2-methoxyethoxy)phenyl)-6-(1H-1,2,4-triazol-3-yl)-1H-imidazo[4,5-
c]pyridin-1-yl)-1-methylpiperidin-3-yl)thiazole-2-carboxamide
5-chloro-N-(5-(2-(2-(2-hydroxyethoxy)phenyl)-6-(1H-1,2,4-triazol-3-yl)-1H-imidazo[4,5-
c]pyridin-1-yl)-1-methylpiperidin-3-yl)thiazole-2-carboxamide
5-chloro-N-(5-(2-(2-(2-methoxyethoxy)phenyl)-6-(1H-1,2,4-triazol-3-yl)-1H-imidazo[4,5-
c]pyridin-1-yl)-1-methylpiperidin-3-yl)thiophene-2-carboxamide
5-chloro-N-(5-(2-(2-(2-hydroxyethoxy)phenyl)-6-(1H-1,2,4-triazol-3-yl)-1H-imidazo[4,5-
c]pyridin-1-yl)-1-methylpiperidin-3-yl)thiophene-2-carboxamide
N-(2-amino-3-(2-(2-fluorophenyl)-6-(1H-1,2,4-triazol-3-yl)-1H-imidazo[4,5-c]pyridin-1-
yl)cyclohexyl)-5-chlorothiazole-2-carboxamide
N-(2-amino-3-(2-(2-fluorophenyl)-6-(1H-1,2,4-triazol-3-yl)-1H-imidazo[4,5-c]pyridin-1-
yl)cyclohexyl)-5-chlorothiophene-2-carboxamide
N-(2-amino-3-(2-(2-fluoro-6-hydroxyphenyl)-6-(1H-1,2,4-triazol-3-yl)-1H-imidazo[4,5-
c]pyridin-1-yl)cyclohexyl)-5-chlorothiazole-2-carboxamide
N-(2-amino-3-(2-(2-fluoro-6-hydroxyphenyl)-6-(1H-1,2,4-triazol-3-yl)-1H-imidazo[4,5-
c]pyridin-1-yl)cyclohexyl)-5-chlorothiophene-2-carboxamide
N-(2-amino-3-(2-isobutyl-6-(1H-1,2,4-triazol-3-yl)-1H-imidazo[4,5-c]pyridin-1-
yl)cyclohexyl)-5-chlorothiazole-2-carboxamide
N-(2-amino-3-(2-isobutyl-6-(1H-1,2,4-triazol-3-yl)-1H-imidazo[4,5-c]pyridin-1-
yl)cyclohexyl)-5-chlorothiophene-2-carboxamide
N-(2-amino-3-(2-(2-fluorophenyl)-6-(pyrimidin-2-yl)-1H-imidazo[4,5-c]pyridin-1-
yl)cyclohexyl)-5-chlorothiazole-2-carboxamide
N-(2-amino-3-(2-(2-fluorophenyl)-6-(pyrimidin-2-yl)-1H-imidazo[4,5-c]pyridin-1-
yl)cyclohexyl)-5-chlorothiophene-2-carboxamide
N-(2-amino-3-(2-(2-fluoro-6-hydroxyphenyl)-6-(pyrimidin-2-yl)-1H-imidazo[4,5-
c]pyridin-1-yl)cyclohexyl)-5-chlorothiazole-2-carboxamide
N-(2-amino-3-(2-(2-fluoro-6-hydroxyphenyl)-6-(pyrimidin-2-yl)-1H-imidazo[4,5-
c]pyridin-1-yl)cyclohexyl)-5-chlorothiophene-2-carboxamide
N-(2-amino-3-(2-isobutyl-6-(pyrimidin-2-yl)-1H-imidazo[4,5-c]pyridin-1-yl)cyclohexyl)-
5-chlorothiazole-2-carboxamide
N-(2-amino-3-(2-isobutyl-6-(pyrimidin-2-yl)-1H-imidazo[4,5-c]pyridin-1-yl)cyclohexyl)-
5-chlorothiophene-2-carboxamide
N-(2-amino-3-(2-(2-fluorophenyl)-6-(oxazol-2-yl)-1H-imidazo[4,5-c]pyridin-1-
yl)cyclohexyl)-5-chlorothiazole-2-carboxamide
N-(2-amino-3-(2-(2-fluorophenyl)-6-(oxazol-2-yl)-1H-imidazo[4,5-c]pyridin-1-
yl)cyclohexyl)-5-chlorothiophene-2-carboxamide
N-(2-amino-3-(2-(2-fluoro-6-hydroxyphenyl)-6-(oxazol-2-yl)-1H-imidazo[4,5-c]pyridin-1-
yl)cyclohexyl)-5-chlorothiazole-2-carboxamide
N-(2-amino-3-(2-(2-fluoro-6-hydroxyphenyl)-6-(oxazol-2-yl)-1H-imidazo[4,5-c]pyridin-1-
yl)cyclohexyl)-5-chlorothiophene-2-carboxamide
N-(2-amino-3-(2-isobutyl-6-(oxazol-2-yl)-1H-imidazo[4,5-c]pyridin-1-yl)cyclohexyl)-5-
chlorothiazole-2-carboxamide
N-(2-amino-3-(2-isobutyl-6-(oxazol-2-yl)-1H-imidazo[4,5-c]pyridin-1-yl)cyclohexyl)-5-
chlorothiophene-2-carboxamide
5-chloro-N-(3-(6-(1-(difluoromethyl)-1H-1,2,4-triazol-3-yl)-2-(2-fluorophenyl)-1H-
imidazo[4,5-c]pyridin-1-yl)-2-hydroxycyclohexyl)thiazole-2-carboxamide
5-chloro-N-(3-(6-(1-(difluoromethyl)-1H-1,2,4-triazol-3-yl)-2-(2-fluorophenyl)-1H-
imidazo[4,5-c]pyridin-1-yl)-2-hydroxycyclohexyl)thiophene-2-carboxamide
5-chloro-N-(3-(6-(1-(difluoromethyl)-1H-1,2,4-triazol-3-yl)-2-(2-fluoro-6-
hydroxyphenyl)-1H-imidazo[4,5-c]pyridin-1-yl)-2-hydroxycyclohexyl)thiazole-2-
carboxamide
5-chloro-N-(3-(6-(1-(difluoromethyl)-1H-1,2,4-triazol-3-yl)-2-(2-fluoro-6-
hydroxyphenyl)-1H-imidazo[4,5-c]pyridin-1-yl)-2-hydroxycyclohexyl)thiophene-2-
carboxamide
5-chloro-N-(3-(6-(1-(difluoromethyl)-1H-1,2,4-triazol-3-yl)-2-isobutyl-1H-imidazo[4,5-
c]pyridin-1-yl)-2-hydroxycyclohexyl)thiazole-2-carboxamide
5-chloro-N-(3-(6-(1-(difluoromethyl)-1H-1,2,4-triazol-3-yl)-2-isobutyl-1H-imidazo[4,5-
c]pyridin-1-yl)-2-hydroxycyclohexyl)thiophene-2-carboxamide
5-chloro-N-(3-(6-(1-(difluoromethyl)-1H-1,2,4-triazol-3-yl)-2-neopentyl-1H-imidazo[4,5-
c]pyridin-1-yl)-2-hydroxycyclohexyl)thiazole-2-carboxamide
5-chloro-N-(3-(6-(1-(difluoromethyl)-1H-1,2,4-triazol-3-yl)-2-neopentyl-1H-imidazo[4,5-
c]pyridin-1-yl)-2-hydroxycyclohexyl)thiophene-2-carboxamide
5-chloro-N-(2-cyano-3-(2-(2-fluorophenyl)-6-(1H-1,2,4-triazol-3-yl)-1H-imidazo[4,5-
c]pyridin-1-yl)cyclohexyl)thiazole-2-carboxamide
5-chloro-N-(2-cyano-3-(2-(2-fluorophenyl)-6-(1H-1,2,4-triazol-3-yl)-1H-imidazo[4,5-
c]pyridin-1-yl)cyclohexyl)thiophene-2-carboxamide
5-chloro-N-(2-cyano-3-(2-(2-fluoro-6-hydroxyphenyl)-6-(1H-1,2,4-triazol-3-yl)-1H-
imidazo[4,5-c]pyridin-1-yl)cyclohexyl)thiazole-2-carboxamide
5-chloro-N-(2-cyano-3-(2-(2-fluoro-6-hydroxyphenyl)-6-(1H-1,2,4-triazol-3-yl)-1H-
imidazo[4,5-c]pyridin-1-yl)cyclohexyl)thiophene-2-carboxamide
5-chloro-N-(2-cyano-3-(2-isobutyl-6-(1H-1,2,4-triazol-3-yl)-1H-imidazo[4,5-c]pyridin-1-
yl)cyclohexyl)thiazole-2-carboxamide
5-chloro-N-(2-cyano-3-(2-isobutyl-6-(1H-1,2,4-triazol-3-yl)-1H-imidazo[4,5-c]pyridin-1-
yl)cyclohexyl)thiophene-2-carboxamide
5-chloro-N-(2-cyano-3-(2-(2-fluorophenyl)-6-(pyrimidin-2-yl)-1H-imidazo[4,5-c]pyridin-
1-yl)cyclohexyl)thiazole-2-carboxamide
5-chloro-N-(2-cyano-3-(2-(2-fluorophenyl)-6-(pyrimidin-2-yl)-1H-imidazo[4,5-c]pyridin-
1-yl)cyclohexyl)thiophene-2-carboxamide
5-chloro-N-(2-cyano-3-(2-(2-fluoro-6-hydroxyphenyl)-6-(pyrimidin-2-yl)-1H-imidazo[4,5-
c]pyridin-1-yl)cyclohexyl)thiazole-2-carboxamide
5-chloro-N-(2-cyano-3-(2-(2-fluoro-6-hydroxyphenyl)-6-(pyrimidin-2-yl)-1H-imidazo[4,5-
c]pyridin-1-yl)cyclohexyl)thiophene-2-carboxamide
5-chloro-N-(2-cyano-3-(2-isobutyl-6-(pyrimidin-2-yl)-1H-imidazo[4,5-c]pyridin-1-
yl)cyclohexyl)thiazole-2-carboxamide
5-chloro-N-(2-cyano-3-(2-isobutyl-6-(pyrimidin-2-yl)-1H-imidazo[4,5-c]pyridin-1-
yl)cyclohexyl)thiophene-2-carboxamide
5-chloro-N-(2-cyano-3-(2-(2-fluorophenyl)-6-(oxazol-2-yl)-1H-imidazo[4,5-c]pyridin-1-
yl)cyclohexyl)thiazole-2-carboxamide
5-chloro-N-(2-cyano-3-(2-(2-fluorophenyl)-6-(oxazol-2-yl)-1H-imidazo[4,5-c]pyridin-1-
yl)cyclohexyl)thiophene-2-carboxamide
5-chloro-N-(2-cyano-3-(2-(2-fluoro-6-hydroxyphenyl)-6-(oxazol-2-yl)-1H-imidazo[4,5-
c]pyridin-1-yl)cyclohexyl)thiazole-2-carboxamide
5-chloro-N-(2-cyano-3-(2-(2-fluoro-6-hydroxyphenyl)-6-(oxazol-2-yl)-1H-imidazo[4,5-
c]pyridin-1-yl)cyclohexyl)thiophene-2-carboxamide
5-chloro-N-(2-cyano-3-(2-isobutyl-6-(oxazol-2-yl)-1H-imidazo[4,5-c]pyridin-1-
yl)cyclohexyl)thiazole-2-carboxamide
5-chloro-N-(2-cyano-3-(2-isobutyl-6-(oxazol-2-yl)-1H-imidazo[4,5-c]pyridin-1-
yl)cyclohexyl)thiophene-2-carboxamide
5-chloro-N-(3-cyano-5-(2-(2-fluorophenyl)-6-(1H-1,2,4-triazol-3-yl)-1H-imidazo[4,5-
c]pyridin-1-yl)cyclohexyl)thiazole-2-carboxamide
5-chloro-N-(3-cyano-5-(2-(2-fluorophenyl)-6-(1H-1,2,4-triazol-3-yl)-1H-imidazo[4,5-
c]pyridin-1-yl)cyclohexyl)thiophene-2-carboxamide
5-chloro-N-(3-cyano-5-(2-(2-fluoro-6-hydroxyphenyl)-6-(1H-1,2,4-triazol-3-yl)-1H-
imidazo[4,5-c]pyridin-1-yl)cyclohexyl)thiazole-2-carboxamide
5-chloro-N-(3-cyano-5-(2-(2-fluoro-6-hydroxyphenyl)-6-(1H-1,2,4-triazol-3-yl)-1H-
imidazo[4,5-c]pyridin-1-yl)cyclohexyl)thiophene-2-carboxamide
5-chloro-N-(3-cyano-5-(2-isobutyl-6-(1H-1,2,4-triazol-3-yl)-1H-imidazo[4,5-c]pyridin-1-
yl)cyclohexyl)thiazole-2-carboxamide
5-chloro-N-(3-cyano-5-(2-isobutyl-6-(1H-1,2,4-triazol-3-yl)-1H-imidazo[4,5-c]pyridin-1-
yl)cyclohexyl)thiophene-2-carboxamide
5-chloro-N-(3-cyano-5-(2-(2-fluorophenyl)-6-(pyrimidin-2-yl)-1H-imidazo[4,5-c]pyridin-
1-yl)cyclohexyl)thiazole-2-carboxamide
5-chloro-N-(3-cyano-5-(2-(2-fluorophenyl)-6-(pyrimidin-2-yl)-1H-imidazo[4,5-c]pyridin-
1-yl)cyclohexyl)thiophene-2-carboxamide
5-chloro-N-(3-cyano-5-(2-(2-fluoro-6-hydroxyphenyl)-6-(pyrimidin-2-yl)-1H-imidazo[4,5-
c]pyridin-1-yl)cyclohexyl)thiazole-2-carboxamide
5-chloro-N-(3-cyano-5-(2-(2-fluoro-6-hydroxyphenyl)-6-(pyrimidin-2-yl)-1H-imidazo[4,5-
c]pyridin-1-yl)cyclohexyl)thiophene-2-carboxamide
5-chloro-N-(3-cyano-5-(2-isobutyl-6-(pyrimidin-2-yl)-1H-imidazo[4,5-c]pyridin-1-
yl)cyclohexyl)thiazole-2-carboxamide
5-chloro-N-(3-cyano-5-(2-isobutyl-6-(pyrimidin-2-yl)-1H-imidazo[4,5-c]pyridin-1-
yl)cyclohexyl)thiophene-2-carboxamide
5-chloro-N-(3-cyano-5-(2-(2-fluorophenyl)-6-(oxazol-2-yl)-1H-imidazo[4,5-c]pyridin-1-
yl)cyclohexyl)thiazole-2-carboxamide
5-chloro-N-(3-cyano-5-(2-(2-fluorophenyl)-6-(oxazol-2-yl)-1H-imidazo[4,5-c]pyridin-1-
yl)cyclohexyl)thiophene-2-carboxamide
5-chloro-N-(3-cyano-5-(2-(2-fluoro-6-hydroxyphenyl)-6-(oxazol-2-yl)-1H-imidazo[4,5-
c]pyridin-1-yl)cyclohexyl)thiazole-2-carboxamide
5-chloro-N-(3-cyano-5-(2-(2-fluoro-6-hydroxyphenyl)-6-(oxazol-2-yl)-1H-imidazo[4,5-
c]pyridin-1-yl)cyclohexyl)thiophene-2-carboxamide
5-chloro-N-(3-cyano-5-(2-isobutyl-6-(oxazol-2-yl)-1H-imidazo[4,5-c]pyridin-1-
yl)cyclohexyl)thiazole-2-carboxamide
5-chloro-N-(3-cyano-5-(2-isobutyl-6-(oxazol-2-yl)-1H-imidazo[4,5-c]pyridin-1-
yl)cyclohexyl)thiophene-2-carboxamide
methyl 3-(5-chlorothiazole-2-carboxamido)-5-(2-(2-fluorophenyl)-6-(1H-1,2,4-triazol-3-
yl)-1H-imidazo[4,5-c]pyridin-1-yl)piperidine-1-carboxylate
5-chloro-N-(5-(2-(2-fluorophenyl)-6-(1H-1,2,4-triazol-3-yl)-1H-imidazo[4,5-c]pyridin-1-
yl)-1,1-dioxidotetrahydro-2H-thiopyran-3-yl)thiazole-2-carboxamide
5-chloro-N-(3-(2-(2-fluorophenyl)-6-(1H-1,2,4-triazol-3-yl)-1H-imidazo[4,5-c]pyridin-1-
yl)-5-(trifluoromethoxy)cyclohexyl) thiazole-2-carboxamide
5-chloro-N-(3-(2-(2-fluorophenyl)-6-(1H-1,2,4-triazol-3-yl)-1H-imidazo[4,5-c]pyridin-1-
yl)-5-(trifluoromethoxy)cyclohexyl) thiophene-2-carboxamide
5-chloro-N-(3-(difluoromethoxy)-5-(2-(2-fluorophenyl)-6-(1H-1,2,4-triazol-3-yl)-1H-
imidazo[4,5-c]pyridin-1-yl)cyclohexyl)thiazole-2-carboxamide
5-chloro-N-(3-(difluoromethoxy)-5-(2-(2-fluorophenyl)-6-(1H-1,2,4-triazol-3-yl)-1H-
imidazo[4,5-c]pyridin-1-yl)cyclohexyl)thiophene-2-carboxamide
5-chloro-N-(3,3-difluoro-5-(2-isobutyl-6-(1H-1,2,4-triazol-3-yl)-1H-imidazo[4,5-
c]pyridin-1-yl)cyclohexyl)thiazole-2-carboxamide
5-chloro-N-(3-(2-(2-(2-methoxyethoxy)phenyl)-6-(1H-1,2,4-triazol-3-yl)-1H-imidazo[4,5-
c]pyridin-1-yl)cyclohexyl)thiophene-2-carboxamide
5-chloro-N-(3-(2-(2-(2-hydroxyethoxy)phenyl)-6-(1H-1,2,4-triazol-3-yl)-1H-imidazo[4,5-
c]pyridin-1-yl)cyclohexyl)thiophene-2-carboxamide

In other embodiments, the compounds described in Table A are the following stereoisomers, or their pharmaceutically acceptable salts, tautomers, and/or isotopologues.

5-chloro-N-((3R,5S)-5-(2-(2-(2-methoxyethoxy)phenyl)-6-(1H-1,2,4-triazol-3-yl)-1H-
imidazo[4,5-c]pyridin-1-yl)-1-methylpiperidin-3-yl)thiazole-2-carboxamide
5-chloro-N-((3R,5S)-5-(2-(2-(2-hydroxyethoxy)phenyl)-6-(1H-1,2,4-triazol-3-yl)-1H-
imidazo[4,5-c]pyridin-1-yl)-1-methylpiperidin-3-yl)thiazole-2-carboxamide
5-chloro-N-((3R,5S)-5-(2-(2-(2-methoxyethoxy)phenyl)-6-(1H-1,2,4-triazol-3-yl)-1H-
imidazo[4,5-c]pyridin-1-yl)-1-methylpiperidin-3-yl)thiophene-2-carboxamide
5-chloro-N-((3R,5S)-5-(2-(2-(2-hydroxyethoxy)phenyl)-6-(1H-1,2,4-triazol-3-yl)-1H-
imidazo[4,5-c]pyridin-1-yl)-1-methylpiperidin-3-yl)thiophene-2-carboxamide
N-((1S,2R,3R)-2-amino-3-(2-(2-fluorophenyl)-6-(1H-1,2,4-triazol-3-yl)-1H-imidazo[4,5-
c]pyridin-1-yl)cyclohexyl)-5-chlorothiazole-2-carboxamide
N-((1S,2R,3R)-2-amino-3-(2-(2-fluorophenyl)-6-(1H-1,2,4-triazol-3-yl)-1H-imidazo[4,5-
c]pyridin-1-yl)cyclohexyl)-5-chlorothiophene-2-carboxamide
N-((1S,2R,3R)-2-amino-3-(2-(2-fluoro-6-hydroxyphenyl)-6-(1H-1,2,4-triazol-3-yl)-1H-
imidazo[4,5-c]pyridin-1-yl)cyclohexyl)-5-chlorothiazole-2-carboxamide
N-((1S,2R,3R)-2-amino-3-(2-(2-fluoro-6-hydroxyphenyl)-6-(1H-1,2,4-triazol-3-yl)-1H-
imidazo[4,5-c]pyridin-1-yl)cyclohexyl)-5-chlorothiophene-2-carboxamide
N-((1S,2R,3R)-2-amino-3-(2-isobutyl-6-(1H-1,2,4-triazol-3-yl)-1H-imidazo[4,5-c]pyridin-
1-yl)cyclohexyl)-5-chlorothiazole-2-carboxamide
N-((1S,2R,3R)-2-amino-3-(2-isobutyl-6-(1H-1,2,4-triazol-3-yl)-1H-imidazo[4,5-c]pyridin-
1-yl)cyclohexyl)-5-chlorothiophene-2-carboxamide
N-((1S,2R,3R)-2-amino-3-(2-(2-fluorophenyl)-6-(pyrimidin-2-yl)-1H-imidazo[4,5-
c]pyridin-1-yl)cyclohexyl)-5-chlorothiazole-2-carboxamide
N-((1S,2R,3R)-2-amino-3-(2-(2-fluorophenyl)-6-(pyrimidin-2-yl)-1H-imidazo[4,5-
c]pyridin-1-yl)cyclohexyl)-5-chlorothiophene-2-carboxamide
N-((1S,2R,3R)-2-amino-3-(2-(2-fluoro-6-hydroxyphenyl)-6-(pyrimidin-2-yl)-1H-
imidazo[4,5-c]pyridin-1-yl)cyclohexyl)-5-chlorothiazole-2-carboxamide
N-((1S,2R,3R)-2-amino-3-(2-(2-fluoro-6-hydroxyphenyl)-6-(pyrimidin-2-yl)-1H-
imidazo[4,5-c]pyridin-1-yl)cyclohexyl)-5-chlorothiophene-2-carboxamide
N-((1S,2R,3R)-2-amino-3-(2-isobutyl-6-(pyrimidin-2-yl)-1H-imidazo[4,5-c]pyridin-1-
yl)cyclohexyl)-5-chlorothiazole-2-carboxamide
N-((1S,2R,3R)-2-amino-3-(2-isobutyl-6-(pyrimidin-2-yl)-1H-imidazo[4,5-c]pyridin-1-
yl)cyclohexyl)-5-chlorothiophene-2-carboxamide
N-((1S,2R,3R)-2-amino-3-(2-(2-fluorophenyl)-6-(oxazol-2-yl)-1H-imidazo[4,5-c]pyridin-
1-yl)cyclohexyl)-5-chlorothiazole-2-carboxamide
N-((1S,2R,3R)-2-amino-3-(2-(2-fluorophenyl)-6-(oxazol-2-yl)-1H-imidazo[4,5-c]pyridin-
1-yl)cyclohexyl)-5-chlorothiophene-2-carboxamide
N-((1S,2R,3R)-2-amino-3-(2-(2-fluoro-6-hydroxyphenyl)-6-(oxazol-2-yl)-1H-imidazo[4,5-
c]pyridin-1-yl)cyclohexyl)-5-chlorothiazole-2-carboxamide
N-((1S,2R,3R)-2-amino-3-(2-(2-fluoro-6-hydroxyphenyl)-6-(oxazol-2-yl)-1H-imidazo[4,5-
c]pyridin-1-yl)cyclohexyl)-5-chlorothiophene-2-carboxamide
N-((1S,2R,3R)-2-amino-3-(2-isobutyl-6-(oxazol-2-yl)-1H-imidazo[4,5-c]pyridin-1-
yl)cyclohexyl)-5-chlorothiazole-2-carboxamide
N-((1S,2R,3R)-2-amino-3-(2-isobutyl-6-(oxazol-2-yl)-1H-imidazo[4,5-c]pyridin-1-
yl)cyclohexyl)-5-chlorothiophene-2-carboxamide
5-chloro-N-((1S,2R,3R)-3-(6-(1-(difluoromethyl)-1H-1,2,4-triazol-3-yl)-2-(2-
fluorophenyl)-1H-imidazo[4,5-c]pyridin-1-yl)-2-hydroxycyclohexyl)thiazole-2-
carboxamide
5-chloro-N-((1S,2R,3R)-3-(6-(1-(difluoromethyl)-1H-1,2,4-triazol-3-yl)-2-(2-
fluorophenyl)-1H-imidazo[4,5-c]pyridin-1-yl)-2-hydroxycyclohexyl)thiophene-2-
carboxamide
5-chloro-N-((1S,2R,3R)-3-(6-(1-(difluoromethyl)-1H-1,2,4-triazol-3-yl)-2-(2-fluoro-6-
hydroxyphenyl)-1H-imidazo[4,5-c]pyridin-1-yl)-2-hydroxycyclohexyl)thiazole-2-
carboxamide
5-chloro-N-((1S,2R,3R)-3-(6-(1-(difluoromethyl)-1H-1,2,4-triazol-3-yl)-2-(2-fluoro-6-
hydroxyphenyl)-1H-imidazo[4,5-c]pyridin-1-yl)-2-hydroxycyclohexyl)thiophene-2-
carboxamide
5-chloro-N-((1S,2R,3R)-3-(6-(1-(difluoromethyl)-1H-1,2,4-triazol-3-yl)-2-isobutyl-1H-
imidazo[4,5-c]pyridin-1-yl)-2-hydroxycyclohexyl)thiazole-2-carboxamide
5-chloro-N-((1S,2R,3R)-3-(6-(1-(difluoromethyl)-1H-1,2,4-triazol-3-yl)-2-isobutyl-1H-
imidazo[4,5-c]pyridin-1-yl)-2-hydroxycyclohexyl)thiophene-2-carboxamide
5-chloro-N-((1S,2R,3R)-3-(6-(1-(difluoromethyl)-1H-1,2,4-triazol-3-yl)-2-neopentyl-1H-
imidazo[4,5-c]pyridin-1-yl)-2-hydroxycyclohexyl)thiazole-2-carboxamide
5-chloro-N-((1S,2R,3R)-3-(6-(1-(difluoromethyl)-1H-1,2,4-triazol-3-yl)-2-neopentyl-1H-
imidazo[4,5-c]pyridin-1-yl)-2-hydroxycyclohexyl)thiophene-2-carboxamide
5-chloro-N-((1S,2R,3R)-2-cyano-3-(2-(2-fluorophenyl)-6-(1H-1,2,4-triazol-3-yl)-1H-
imidazo[4,5-c]pyridin-1-yl)cyclohexyl)thiazole-2-carboxamide
5-chloro-N-((1S,2R,3R)-2-cyano-3-(2-(2-fluorophenyl)-6-(1H-1,2,4-triazol-3-yl)-1H-
imidazo[4,5-c]pyridin-1-yl)cyclohexyl)thiophene-2-carboxamide
5-chloro-N-((1S,2R,3R)-2-cyano-3-(2-(2-fluoro-6-hydroxyphenyl)-6-(1H-1,2,4-triazol-3-
yl)-1H-imidazo[4,5-c]pyridin-1-yl)cyclohexyl)thiazole-2-carboxamide
5-chloro-N-((1S,2R,3R)-2-cyano-3-(2-(2-fluoro-6-hydroxyphenyl)-6-(1H-1,2,4-triazol-3-
yl)-1H-imidazo[4,5-c]pyridin-1-yl)cyclohexyl)thiophene-2-carboxamide
5-chloro-N-((1S,2R,3R)-2-cyano-3-(2-isobutyl-6-(1H-1,2,4-triazol-3-yl)-1H-imidazo[4,5-
c]pyridin-1-yl)cyclohexyl)thiazole-2-carboxamide
5-chloro-N-((1S,2R,3R)-2-cyano-3-(2-isobutyl-6-(1H-1,2,4-triazol-3-yl)-1H-imidazo[4,5-
c]pyridin-1-yl)cyclohexyl)thiophene-2-carboxamide
5-chloro-N-((1S,2R,3R)-2-cyano-3-(2-(2-fluorophenyl)-6-(pyrimidin-2-yl)-1H-
imidazo[4,5-c]pyridin-1-yl)cyclohexyl)thiazole-2-carboxamide
5-chloro-N-((1S,2R,3R)-2-cyano-3-(2-(2-fluorophenyl)-6-(pyrimidin-2-yl)-1H-
imidazo[4,5-c]pyridin-1-yl)cyclohexyl)thiophene-2-carboxamide
5-chloro-N-((1S,2R,3R)-2-cyano-3-(2-(2-fluoro-6-hydroxyphenyl)-6-(pyrimidin-2-yl)-1H-
imidazo[4,5-c]pyridin-1-yl)cyclohexyl)thiazole-2-carboxamide
5-chloro-N-((1S,2R,3R)-2-cyano-3-(2-(2-fluoro-6-hydroxyphenyl)-6-(pyrimidin-2-yl)-1H-
imidazo[4,5-c]pyridin-1-yl)cyclohexyl)thiophene-2-carboxamide
5-chloro-N-((1S,2R,3R)-2-cyano-3-(2-isobutyl-6-(pyrimidin-2-yl)-1H-imidazo[4,5-
c]pyridin-1-yl)cyclohexyl)thiazole-2-carboxamide
5-chloro-N-((1S,2R,3R)-2-cyano-3-(2-isobutyl-6-(pyrimidin-2-yl)-1H-imidazo[4,5-
c]pyridin-1-yl)cyclohexyl)thiophene-2-carboxamide
5-chloro-N-((1S,2R,3R)-2-cyano-3-(2-(2-fluorophenyl)-6-(oxazol-2-yl)-1H-imidazo[4,5-
c]pyridin-1-yl)cyclohexyl)thiazole-2-carboxamide
5-chloro-N-((1S,2R,3R)-2-cyano-3-(2-(2-fluorophenyl)-6-(oxazol-2-yl)-1H-imidazo[4,5-
c]pyridin-1-yl)cyclohexyl)thiophene-2-carboxamide
5-chloro-N-((1S,2R,3R)-2-cyano-3-(2-(2-fluoro-6-hydroxyphenyl)-6-(oxazol-2-yl)-1H-
imidazo[4,5-c]pyridin-1-yl)cyclohexyl)thiazole-2-carboxamide
5-chloro-N-((1S,2R,3R)-2-cyano-3-(2-(2-fluoro-6-hydroxyphenyl)-6-(oxazol-2-yl)-1H-
imidazo[4,5-c]pyridin-1-yl)cyclohexyl)thiophene-2-carboxamide
5-chloro-N-((1S,2R,3R)-2-cyano-3-(2-isobutyl-6-(oxazol-2-yl)-1H-imidazo[4,5-c]pyridin-
1-yl)cyclohexyl)thiazole-2-carboxamide
5-chloro-N-((1S,2R,3R)-2-cyano-3-(2-isobutyl-6-(oxazol-2-yl)-1H-imidazo[4,5-c]pyridin-
1-yl)cyclohexyl)thiophene-2-carboxamide
5-chloro-N-((1S,3S,5R)-3-cyano-5-(2-(2-fluorophenyl)-6-(1H-1,2,4-triazol-3-yl)-1H-
imidazo[4,5-c]pyridin-1-yl)cyclohexyl)thiazole-2-carboxamide
5-chloro-N-((1S,3S,5R)-3-cyano-5-(2-(2-fluorophenyl)-6-(1H-1,2,4-triazol-3-yl)-1H-
imidazo[4,5-c]pyridin-1-yl)cyclohexyl)thiophene-2-carboxamide
5-chloro-N-((1S,3S,5R)-3-cyano-5-(2-(2-fluoro-6-hydroxyphenyl)-6-(1H-1,2,4-triazol-3-
yl)-1H-imidazo[4,5-c]pyridin-1-yl)cyclohexyl)thiazole-2-carboxamide
5-chloro-N-((1S,3S,5R)-3-cyano-5-(2-(2-fluoro-6-hydroxyphenyl)-6-(1H-1,2,4-triazol-3-
yl)-1H-imidazo[4,5-c]pyridin-1-yl)cyclohexyl)thiophene-2-carboxamide
5-chloro-N-((1S,3S,5R)-3-cyano-5-(2-isobutyl-6-(1H-1,2,4-triazol-3-yl)-1H-imidazo[4,5-
c]pyridin-1-yl)cyclohexyl)thiazole-2-carboxamide
5-chloro-N-((1S,3S,5R)-3-cyano-5-(2-isobutyl-6-(1H-1,2,4-triazol-3-yl)-1H-imidazo[4,5-
c]pyridin-1-yl)cyclohexyl)thiophene-2-carboxamide
5-chloro-N-((1S,3S,5R)-3-cyano-5-(2-(2-fluorophenyl)-6-(pyrimidin-2-yl)-1H-
imidazo[4,5-c]pyridin-1-yl)cyclohexyl)thiazole-2-carboxamide
5-chloro-N-((1S,3S,5R)-3-cyano-5-(2-(2-fluorophenyl)-6-(pyrimidin-2-yl)-1H-
imidazo[4,5-c]pyridin-1-yl)cyclohexyl)thiophene-2-carboxamide
5-chloro-N-((1S,3S,5R)-3-cyano-5-(2-(2-fluoro-6-hydroxyphenyl)-6-(pyrimidin-2-yl)-1H-
imidazo[4,5-c]pyridin-1-yl)cyclohexyl)thiazole-2-carboxamide
5-chloro-N-((1S,3S,5R)-3-cyano-5-(2-(2-fluoro-6-hydroxyphenyl)-6-(pyrimidin-2-yl)-1H-
imidazo[4,5-c]pyridin-1-yl)cyclohexyl)thiophene-2-carboxamide
5-chloro-N-((1S,3S,5R)-3-cyano-5-(2-isobutyl-6-(pyrimidin-2-yl)-1H-imidazo[4,5-
c]pyridin-1-yl)cyclohexyl)thiazole-2-carboxamide
5-chloro-N-((1S,3S,5R)-3-cyano-5-(2-isobutyl-6-(pyrimidin-2-yl)-1H-imidazo[4,5-
c]pyridin-1-yl)cyclohexyl)thiophene-2-carboxamide
5-chloro-N-((1S,3S,5R)-3-cyano-5-(2-(2-fluorophenyl)-6-(oxazol-2-yl)-1H-imidazo[4,5-
c]pyridin-1-yl)cyclohexyl)thiazole-2-carboxamide
5-chloro-N-((1S,3S,5R)-3-cyano-5-(2-(2-fluorophenyl)-6-(oxazol-2-yl)-1H-imidazo[4,5-
c]pyridin-1-yl)cyclohexyl)thiophene-2-carboxamide
5-chloro-N-((1S,3S,5R)-3-cyano-5-(2-(2-fluoro-6-hydroxyphenyl)-6-(oxazol-2-yl)-1H-
imidazo[4,5-c]pyridin-1-yl)cyclohexyl)thiazole-2-carboxamide
5-chloro-N-((1S,3S,5R)-3-cyano-5-(2-(2-fluoro-6-hydroxyphenyl)-6-(oxazol-2-yl)-1H-
imidazo[4,5-c]pyridin-1-yl)cyclohexyl)thiophene-2-carboxamide
5-chloro-N-((1S,3S,5R)-3-cyano-5-(2-isobutyl-6-(oxazol-2-yl)-1H-imidazo[4,5-c]pyridin-
1-yl)cyclohexyl)thiazole-2-carboxamide
5-chloro-N-((1S,3S,5R)-3-cyano-5-(2-isobutyl-6-(oxazol-2-yl)-1H-imidazo[4,5-c]pyridin-
1-yl)cyclohexyl)thiophene-2-carboxamide
methyl (3R,5S)-3-(5-chlorothiazole-2-carboxamido)-5-(2-(2-fluorophenyl)-6-(1H-1,2,4-
triazol-3-yl)-1H-imidazo[4,5-c]pyridin-1-yl)piperidine-1-carboxylate
5-chloro-N-((3R,5S)-5-(2-(2-fluorophenyl)-6-(1H-1,2,4-triazol-3-yl)-1H-imidazo[4,5-
c]pyridin-1-yl)-1,1-dioxidotetrahydro-2H-thiopyran-3-yl)thiazole-2-carboxamide
5-chloro-N-((1R,3S,5S)-3-(2-(2-fluorophenyl)-6-(1H-1,2,4-triazol-3-yl)-1H-imidazo[4,5-
c]pyridin-1-yl)-5-(trifluoromethoxy)cyclohexyl) thiazole-2-carboxamide
5-chloro-N-((1R,3S,5S)-3-(2-(2-fluorophenyl)-6-(1H-1,2,4-triazol-3-yl)-1H-imidazo[4,5-
c]pyridin-1-yl)-5-(trifluoromethoxy)cyclohexyl) thiophene-2-carboxamide
5-chloro-N-((1R,3S,5S)-3-(difluoromethoxy)-5-(2-(2-fluorophenyl)-6-(1H-1,2,4-triazol-3-
yl)-1H-imidazo[4,5-c]pyridin-1-yl)cyclohexyl)thiazole-2-carboxamide
5-chloro-N-((1R,3S,5S)-3-(difluoromethoxy)-5-(2-(2-fluorophenyl)-6-(1H-1,2,4-triazol-3-
yl)-1H-imidazo[4,5-c]pyridin-1-yl)cyclohexyl)thiophene-2-carboxamide
5-chloro-N-((1R,5S)-3,3-difluoro-5-(2-isobutyl-6-(1H-1,2,4-triazol-3-yl)-1H-imidazo[4,5-
c]pyridin-1-yl)cyclohexyl)thiazole-2-carboxamide
5-chloro-N-((1S,3R)-3-(2-(2-(2-methoxyethoxy)phenyl)-6-(1H-1,2,4-triazol-3-yl)-1H-
imidazo[4,5-c]pyridin-1-yl)cyclohexyl)thiophene-2-carboxamide
5-chloro-N-((1S,3R)-3-(2-(2-(2-hydroxyethoxy)phenyl)-6-(1H-1,2,4-triazol-3-yl)-1H-
imidazo[4,5-c]pyridin-1-yl)cyclohexyl)thiophene-2-carboxamide
N-((1S,2S,3R)-2-amino-3-(2-(2-fluorophenyl)-6-(1H-1,2,4-triazol-3-yl)-1H-imidazo[4,5-
c]pyridin-1-yl)cyclohexyl)-5-chlorothiazole-2-carboxamide
N-((1S,2S,3R)-2-amino-3-(2-(2-fluoro-6-hydroxyphenyl)-6-(1H-1,2,4-triazol-3-yl)-1H-
imidazo[4,5-c]pyridin-1-yl)cyclohexyl)-5-chlorothiophene-2-carboxamide
N-((1S,2S,3R)-2-amino-3-(2-(2-fluoro-6-hydroxyphenyl)-6-(1H-1,2,4-triazol-3-yl)-1H-
imidazo[4,5-c]pyridin-1-yl)cyclohexyl)-5-chlorothiazole-2-carboxamide
N-((1S,2S,3R)-2-amino-3-(2-(2-fluoro-6-hydroxyphenyl)-6-(1H-1,2,4-triazol-3-yl)-1H-
imidazo[4,5-c]pyridin-1-yl)cyclohexyl)-5-chlorothiophene-2-carboxamide
N-((1S,2S,3R)-2-amino-3-(2-isobutyl-6-(1H-1,2,4-triazol-3-yl)-1H-imidazo[4,5-c]pyridin-
1-yl)cyclohexyl)-5-chlorothiazole-2-carboxamide
N-((1S,2S,3R)-2-amino-3-(2-isobutyl-6-(1H-1,2,4-triazol-3-yl)-1H-imidazo[4,5-c]pyridin-
1-yl)cyclohexyl)-5-chlorothiophene-2-carboxamide
N-((1S,2S,3R)-2-amino-3-(2-(2-fluorophenyl)-6-(pyrimidin-2-yl)-1H-imidazo[4,5-
c]pyridin-1-yl)cyclohexyl)-5-chlorothiazole-2-carboxamide
N-((1S,2S,3R)-2-amino-3-(2-(2-fluorophenyl)-6-(pyrimidin-2-yl)-1H-imidazo[4,5-
c]pyridin-1-yl)cyclohexyl)-5-chlorothiophene-2-carboxamide
N-((1S,2S,3R)-2-amino-3-(2-(2-fluoro-6-hydroxyphenyl)-6-(pyrimidin-2-yl)-1H-
imidazo[4,5-c]pyridin-1-yl)cyclohexyl)-5-chlorothiazole-2-carboxamide
N-((1S,2S,3R)-2-amino-3-(2-(2-fluoro-6-hydroxyphenyl)-6-(pyrimidin-2-yl)-1H-
imidazo[4,5-c]pyridin-1-yl)cyclohexyl)-5-chlorothiophene-2-carboxamide
N-((1S,2S,3R)-2-amino-3-(2-isobutyl-6-(pyrimidin-2-yl)-1H-imidazo[4,5-c]pyridin-1-
yl)cyclohexyl)-5-chlorothiazole-2-carboxamide
N-((1S,2S,3R)-2-amino-3-(2-isobutyl-6-(pyrimidin-2-yl)-1H-imidazo[4,5-c]pyridin-1-
yl)cyclohexyl)-5-chlorothiophene-2-carboxamide
N-((1S,2S,3R)-2-amino-3-(2-(2-fluorophenyl)-6-(oxazol-2-yl)-1H-imidazo[4,5-c]pyridin-
1-yl)cyclohexyl)-5-chlorothiazole-2-carboxamide
N-((1S,2S,3R)-2-amino-3-(2-(2-fluorophenyl)-6-(oxazol-2-yl)-1H-imidazo[4,5-c]pyridin-
1-yl)cyclohexyl)-5-chlorothiophene-2-carboxamide
N-((1S,2S,3R)-2-amino-3-(2-(2-fluoro-6-hydroxyphenyl)-6-(oxazol-2-yl)-1H-imidazo[4,5-
c]pyridin-1-yl)cyclohexyl)-5-chlorothiazole-2-carboxamide
N-((1S,2S,3R)-2-amino-3-(2-(2-fluoro-6-hydroxyphenyl)-6-(oxazol-2-yl)-1H-imidazo[4,5-
c]pyridin-1-yl)cyclohexyl)-5-chlorothiophene-2-carboxamide
N-((1S,2S,3R)-2-amino-3-(2-isobutyl-6-(oxazol-2-yl)-1H-imidazo[4,5-c]pyridin-1-
yl)cyclohexyl)-5-chlorothiazole-2-carboxamide
N-((1S,2S,3R)-2-amino-3-(2-isobutyl-6-(oxazol-2-yl)-1H-imidazo[4,5-c]pyridin-1-
yl)cyclohexyl)-5-chlorothiophene-2-carboxamide
5-chloro-N-((1S,2S,3R)-3-(6-(1-(difluoromethyl)-1H-1,2,4-triazol-3-yl)-2-(2-
fluorophenyl)-1H-imidazo[4,5-c]pyridin-1-yl)-2-hydroxycyclohexyl)thiazole-2-
carboxamide
5-chloro-N-((1S,2S,3R)-3-(6-(1-(difluoromethyl)-1H-1,2,4-triazol-3-yl)-2-(2-
fluorophenyl)-1H-imidazo[4,5-c]pyridin-1-yl)-2-hydroxycyclohexyl)thiophene-2-
carboxamide
5-chloro-N-((1S,2S,3R)-3-(6-(1-(difluoromethyl)-1H-1,2,4-triazol-3-yl)-2-(2-fluoro-6-
hydroxyphenyl)-1H-imidazo[4,5-c]pyridin-1-yl)-2-hydroxycyclohexyl)thiazole-2-
carboxamide
5-chloro-N-((1S,2S,3R)-3-(6-(1-(difluoromethyl)-1H-1,2,4-triazol-3-yl)-2-(2-fluoro-6-
hydroxyphenyl)-1H-imidazo[4,5-c]pyridin-1-yl)-2-hydroxycyclohexyl)thiophene-2-
carboxamide
5-chloro-N-((1S,2S,3R)-3-(6-(1-(difluoromethyl)-1H-1,2,4-triazol-3-yl)-2-isobutyl-1H-
imidazo[4,5-c]pyridin-1-yl)-2-hydroxycyclohexyl)thiazole-2-carboxamide
5-chloro-N-((1S,2S,3R)-3-(6-(1-(difluoromethyl)-1H-1,2,4-triazol-3-yl)-2-isobutyl-1H-
imidazo[4,5-c]pyridin-1-yl)-2-hydroxycyclohexyl)thiophene-2-carboxamide
5-chloro-N-((1S,2S,3R)-3-(6-(1-(difluoromethyl)-1H-1,2,4-triazol-3-yl)-2-neopentyl-1H-
imidazo[4,5-c]pyridin-1-yl)-2-hydroxycyclohexyl)thiazole-2-carboxamide
5-chloro-N-((1S,2S,3R)-3-(6-(1-(difluoromethyl)-1H-1,2,4-triazol-3-yl)-2-neopentyl-1H-
imidazo[4,5-c]pyridin-1-yl)-2-hydroxycyclohexyl)thiophene-2-carboxamide
5-chloro-N-((1S,2S,3R)-2-cyano-3-(2-(2-fluorophenyl)-6-(1H-1,2,4-triazol-3-yl)-1H-
imidazo[4,5-c]pyridin-1-yl)cyclohexyl)thiazole-2-carboxamide
5-chloro-N-((1S,2S,3R)-2-cyano-3-(2-(2-fluorophenyl)-6-(1H-1,2,4-triazol-3-yl)-1H-
imidazo[4,5-c]pyridin-1-yl)cyclohexyl)thiophene-2-carboxamide
5-chloro-N-((1S,2S,3R)-2-cyano-3-(2-(2-fluoro-6-hydroxyphenyl)-6-(1H-1,2,4-triazol-3-
yl)-1H-imidazo[4,5-c]pyridin-1-yl)cyclohexyl)thiazole-2-carboxamide
5-chloro-N-((1S,2S,3R)-2-cyano-3-(2-(2-fluoro-6-hydroxyphenyl)-6-(1H-1,2,4-triazol-3-
yl)-1H-imidazo[4,5-c]pyridin-1-yl)cyclohexyl)thiophene-2-carboxamide
5-chloro-N-((1S,2S,3R)-2-cyano-3-(2-isobutyl-6-(1H-1,2,4-triazol-3-yl)-1H-imidazo[4,5-
c]pyridin-1-yl)cyclohexyl)thiazole-2-carboxamide
5-chloro-N-((1S,2S,3R)-2-cyano-3-(2-isobutyl-6-(1H-1,2,4-triazol-3-yl)-1H-imidazo[4,5-
c]pyridin-1-yl)cyclohexyl)thiophene-2-carboxamide
5-chloro-N-((1S,2S,3R)-2-cyano-3-(2-(2-fluorophenyl)-6-(pyrimidin-2-yl)-1H-
imidazo[4,5-c]pyridin-1-yl)cyclohexyl)thiazole-2-carboxamide
5-chloro-N-((1S,2S,3R)-2-cyano-3-(2-(2-fluorophenyl)-6-(pyrimidin-2-yl)-1H-
imidazo[4,5-c]pyridin-1-yl)cyclohexyl)thiophene-2-carboxamide
5-chloro-N-((1S,2S,3R)-2-cyano-3-(2-(2-fluoro-6-hydroxyphenyl)-6-(pyrimidin-2-yl)-1H-
imidazo[4,5-c]pyridin-1-yl)cyclohexyl)thiazole-2-carboxamide
5-chloro-N-((1S,2S,3R)-2-cyano-3-(2-(2-fluoro-6-hydroxyphenyl)-6-(pyrimidin-2-yl)-1H-
imidazo[4,5-c]pyridin-1-yl)cyclohexyl)thiophene-2-carboxamide
5-chloro-N-((1S,2S,3R)-2-cyano-3-(2-isobutyl-6-(pyrimidin-2-yl)-1H-imidazo[4,5-
c]pyridin-1-yl)cyclohexyl)thiazole-2-carboxamide
5-chloro-N-((1S,2S,3R)-2-cyano-3-(2-isobutyl-6-(pyrimidin-2-yl)-1H-imidazo[4,5-
c]pyridin-1-yl)cyclohexyl)thiophene-2-carboxamide
5-chloro-N-((1S,2S,3R)-2-cyano-3-(2-(2-fluorophenyl)-6-(oxazol-2-yl)-1H-imidazo[4,5-
c]pyridin-1-yl)cyclohexyl)thiazole-2-carboxamide
5-chloro-N-((1S,2S,3R)-2-cyano-3-(2-(2-fluorophenyl)-6-(oxazol-2-yl)-1H-imidazo[4,5-
c]pyridin-1-yl)cyclohexyl)thiophene-2-carboxamide
5-chloro-N-((1S,2S,3R)-2-cyano-3-(2-(2-fluoro-6-hydroxyphenyl)-6-(oxazol-2-yl)-1H-
imidazo[4,5-c]pyridin-1-yl)cyclohexyl)thiazole-2-carboxamide
5-chloro-N-((1S,2S,3R)-2-cyano-3-(2-(2-fluoro-6-hydroxyphenyl)-6-(oxazol-2-yl)-1H-
imidazo[4,5-c]pyridin-1-yl)cyclohexyl)thiophene-2-carboxamide
5-chloro-N-((1S,2S,3R)-2-cyano-3-(2-isobutyl-6-(oxazol-2-yl)-1H-imidazo[4,5-c]pyridin-
1-yl)cyclohexyl)thiazole-2-carboxamide
5-chloro-N-((1S,2S,3R)-2-cyano-3-(2-isobutyl-6-(oxazol-2-yl)-1H-imidazo[4,5-c]pyridin-
1-yl)cyclohexyl)thiophene-2-carboxamide
5-chloro-N-((1S,3R,5R)-3-cyano-5-(2-(2-fluorophenyl)-6-(1H-1,2,4-triazol-3-yl)-1H-
imidazo[4,5-c]pyridin-1-yl)cyclohexyl)thiazole-2-carboxamide
5-chloro-N-((1S,3R,5R)-3-cyano-5-(2-(2-fluorophenyl)-6-(1H-1,2,4-triazol-3-yl)-1H-
imidazo[4,5-c]pyridin-1-yl)cyclohexyl)thiophene-2-carboxamide
5-chloro-N-((1S,3R,5R)-3-cyano-5-(2-(2-fluoro-6-hydroxyphenyl)-6-(1H-1,2,4-triazol-3-
yl)-1H-imidazo[4,5-c]pyridin-1-yl)cyclohexyl)thiazole-2-carboxamide
5-chloro-N-((1S,3R,5R)-3-cyano-5-(2-(2-fluoro-6-hydroxyphenyl)-6-(1H-1,2,4-triazol-3-
yl)-1H-imidazo[4,5-c]pyridin-1-yl)cyclohexyl)thiophene-2-carboxamide
5-chloro-N-((1S,3R,5R)-3-cyano-5-(2-isobutyl-6-(1H-1,2,4-triazol-3-yl)-1H-imidazo[4,5-
c]pyridin-1-yl)cyclohexyl)thiazole-2-carboxamide
5-chloro-N-((1S,3R,5R)-3-cyano-5-(2-isobutyl-6-(1H-1,2,4-triazol-3-yl)-1H-imidazo[4,5-
c]pyridin-1-yl)cyclohexyl)thiophene-2-carboxamide
5-chloro-N-((1S,3R,5R)-3-cyano-5-(2-(2-fluorophenyl)-6-(pyrimidin-2-yl)-1H-
imidazo[4,5-c]pyridin-1-yl)cyclohexyl)thiazole-2-carboxamide
5-chloro-N-((1S,3R,5R)-3-cyano-5-(2-(2-fluorophenyl)-6-(pyrimidin-2-yl)-1H-
imidazo[4,5-c]pyridin-1-yl)cyclohexyl)thiophene-2-carboxamide
5-chloro-N-((1S,3R,5R)-3-cyano-5-(2-(2-fluoro-6-hydroxyphenyl)-6-(pyrimidin-2-yl)-1H-
imidazo[4,5-c]pyridin-1-yl)cyclohexyl)thiazole-2-carboxamide
5-chloro-N-((1S,3R,5R)-3-cyano-5-(2-(2-fluoro-6-hydroxyphenyl)-6-(pyrimidin-2-yl)-1H-
imidazo[4,5-c]pyridin-1-yl)cyclohexyl)thiophene-2-carboxamide
5-chloro-N-((1S,3R,5R)-3-cyano-5-(2-isobutyl-6-(pyrimidin-2-yl)-1H-imidazo[4,5-
c]pyridin-1-yl)cyclohexyl)thiazole-2-carboxamide
5-chloro-N-((1S,3R,5R)-3-cyano-5-(2-isobutyl-6-(pyrimidin-2-yl)-1H-imidazo[4,5-
c]pyridin-1-yl)cyclohexyl)thiophene-2-carboxamide
5-chloro-N-((1S,3R,5R)-3-cyano-5-(2-(2-fluorophenyl)-6-(oxazol-2-yl)-1H-imidazo[4,5-
c]pyridin-1-yl)cyclohexyl)thiazole-2-carboxamide
5-chloro-N-((1S,3R,5R)-3-cyano-5-(2-(2-fluorophenyl)-6-(oxazol-2-yl)-1H-imidazo[4,5-
c]pyridin-1-yl)cyclohexyl)thiophene-2-carboxamide
5-chloro-N-((1S,3R,5R)-3-cyano-5-(2-(2-fluoro-6-hydroxyphenyl)-6-(oxazol-2-yl)-1H-
imidazo[4,5-c]pyridin-1-yl)cyclohexyl)thiazole-2-carboxamide
5-chloro-N-((1S,3R,5R)-3-cyano-5-(2-(2-fluoro-6-hydroxyphenyl)-6-(oxazol-2-yl)-1H-
imidazo[4,5-c]pyridin-1-yl)cyclohexyl)thiophene-2-carboxamide
5-chloro-N-((1S,3R,5R)-3-cyano-5-(2-isobutyl-6-(oxazol-2-yl)-1H-imidazo[4,5-c]pyridin-
1-yl)cyclohexyl)thiazole-2-carboxamide
5-chloro-N-((1S,3R,5R)-3-cyano-5-(2-isobutyl-6-(oxazol-2-yl)-1H-imidazo[4,5-c]pyridin-
1-yl)cyclohexyl)thiophene-2-carboxamide
methyl (3S,5R)-3-(5-chlorothiazole-2-carboxamido)-5-(2-(2-fluorophenyl)-6-(1H-1,2,4-
triazol-3-yl)-1H-imidazo[4,5-c]pyridin-1-yl)piperidine-1-carboxylate
5-chloro-N-((3S,5R)-5-(2-(2-fluorophenyl)-6-(1H-1,2,4-triazol-3-yl)-1H-imidazo[4,5-
c]pyridin-1-yl)-1,1-dioxidotetrahydro-2H-thiopyran-3-yl)thiazole-2-carboxamide

In some embodiments, the disclosure provides specific examples of Formulas IIA and IIB, and their pharmaceutically acceptable salts, tautomers, and/or isotopologues as set forth in Table 2 below.

TABLE 2

| Ex. | | |
|---|---|---|
| No. | Structure | Chemical Name |
| 266 | 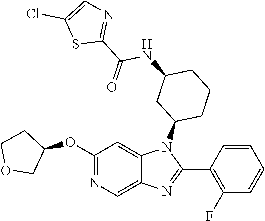 [SMILES: O=C(N[C@H]1CCC[C@@H](n2c(-c3ccccc3F)nc3cnc(O[C@@H]4CCOC4)cc32)C1)c1ncc(Cl)s1] | 5-chloro-N-((1S,3R)-3- (2-(2-fluorophenyl)-6- (((R)-tetrahydrofuran- 3-yl)oxy)-1H- imidazo[4,5-c]pyridin-1- yl)cyclohexyl)thiazole-2- carboxamide |
| 267 | 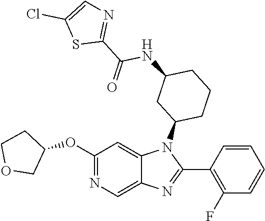 [SMILES: O=C(N[C@H]1CCC[C@@H](n2c(-c3ccccc3F)nc3cnc(O[C@H]4CCOC4)cc32)C1)c1ncc(Cl)s1] | 5-chloro-N-((1S,3R)-3- (2-(2-fluorophenyl)-6- (((S)-tetrahydrofuran- 3-yl)oxy)-1H- imidazo[4,5-c]pyridin-1- yl)cyclohexyl)thiazole-2- carboxamide |
| 268 | 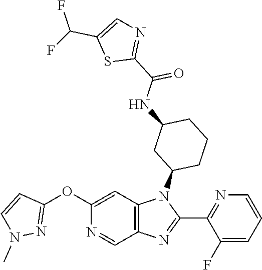 [SMILES: Cn1ccc(Oc2cc3c(cn2)nc(-c2ncccc2F)n3[C@@H]2CCC[C@H](NC(=O)c3ncc(C(F)F)s3)C2)n1] | 5-(difluoromethyl)-N- ((1S,3R)-3-(2-(3- fluoropyridin-2-yl)-6-((1- methyl-1H-pyrazol-3-yl) oxy)-1H-imidazo [4,5-c]pyridin-1- yl)cyclohexyl)thiazole-2- carboxamide |

In some embodiments, the disclosure provides specific examples of Formulas IIIA and IIIB, and their pharmaceutically acceptable salts, tautomers, and/or isotopologues as set forth in Table 3 below.

TABLE 3

| Ex. | | |
|---|---|---|
| No. | Structure | Chemical Name |
| 269 | 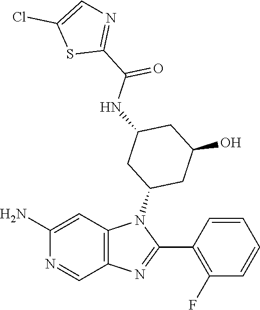 [SMILES: Nc1cc2c(cn1)nc(-c1ccccc1F)n2[C@H]1C[C@H](O)C[C@@H](NC(=O)c2ncc(Cl)s2)C1] | N-((1S,3R,5R)-3-(6-amino-2-(2- fluorophenyl)-1H-imidazo[4,5- c]pyridin-1-yl)-5- hydroxycyclohexyl)-5- chlorothiazole-2-carboxamide |
| 270 | 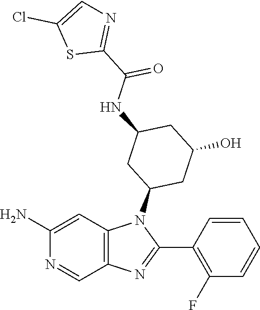 [SMILES: Nc1cc2c(cn1)nc(-c1ccccc1F)n2[C@@H]1C[C@@H](O)C[C@H](NC(=O)c2ncc(Cl)s2)C1] | N-((1R,3S,5S)-3-(6-amino-2-(2- fluorophenyl)-1H-imidazo[4,5- c]pyridin-1-yl)-5- hydroxycyclohexyl)-5- chlorothiazole-2-carboxamide |
| 271 | 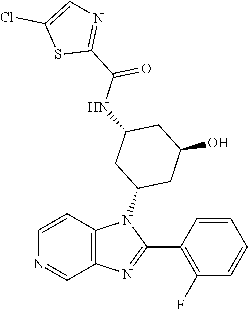 [SMILES: O=C(N[C@@H]1C[C@@H](O)C[C@H](n2c(-c3ccccc3F)nc3cnccc32)C1)c1ncc(Cl)s1] | 5-chloro-N-((1S,3R,5R)-3-(2-(2- fluorophenyl)-1H-imidazo[4,5- c]pyridin-1-yl)-5- hydroxycyclohexyl)thiazole-2- carboxamide |
| 272 | 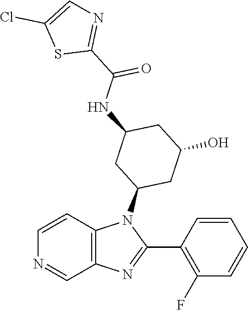 [SMILES: O=C(N[C@H]1C[C@H](O)C[C@@H](n2c(-c3ccccc3F)nc3cnccc32)C1)c1ncc(Cl)s1] | 5-chloro-N-((1R,3S,5S)-3-(2-(2- fluorophenyl)-1H-imidazo[4,5- c]pyridin-1-yl)-5- hydroxycyclohexyl)thiazole-2- carboxamide |
| 273 | 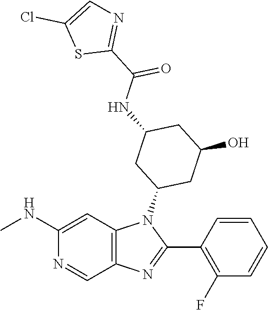 [SMILES: CNc1cc2c(cn1)nc(-c1ccccc1F)n2[C@H]1C[C@H](O)C[C@@H](NC(=O)c2ncc(Cl)s2)C1] | 5-chloro-N-((1S,3R,5R)-3-(2-(2- fluorophenyl)-6-(methylamino)- 1H-imidazo[4,5-c]pyridin-1-yl)-5- hydroxycyclohexyl)thiazole-2- carboxamide |
| 274 | 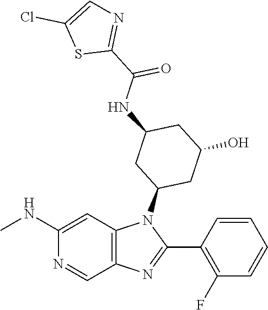 [SMILES: CNc1cc2c(cn1)nc(-c1ccccc1F)n2[C@@H]1C[C@@H](O)C[C@H](NC(=O)c2ncc(Cl)s2)C1] | 5-chloro-N-((1R,3S,5S)-3-(2-(2- fluorophenyl)-6-(methylamino)- 1H-imidazo[4,5-c]pyridin-1-yl)-5- hydroxycyclohexyl)thiazole-2- carboxamide |
| 275 | 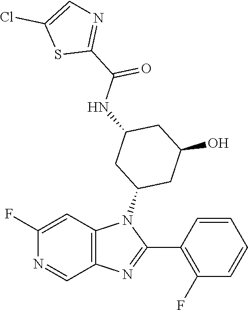 [SMILES: O=C(N[C@@H]1C[C@@H](O)C[C@H](n2c(-c3ccccc3F)nc3cnc(F)cc32)C1)c1ncc(Cl)s1] | 5-chloro-N-((1S,3R,5R)-3-(6- fluoro-2-(2-fluorophenyl)-1H- imidazo[4,5-c]pyridin-1-yl)-5- hydroxycyclohexyl)thiazole-2- carboxamide |
| 276 | 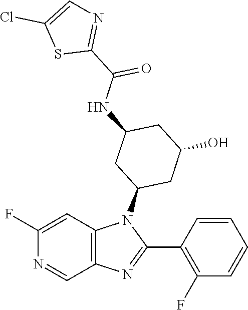 [SMILES: O=C(N[C@H]1C[C@H](O)C[C@@H](n2c(-c3ccccc3F)nc3cnc(F)cc32)C1)c1ncc(Cl)s1] | 5-chloro-N-((1R,3S,5S)-3-(6- fluoro-2-(2-fluorophenyl)-1H- imidazo[4,5-c]pyridin-1-yl)-5- hydroxycyclohexyl)thiazole-2- carboxamide |
| 277 | 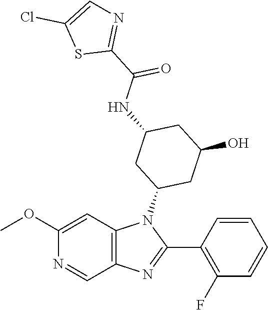 [SMILES: COc1cc2c(cn1)nc(-c1ccccc1F)n2[C@H]1C[C@H](O)C[C@@H](NC(=O)c2ncc(Cl)s2)C1] | 5-chloro-N-((1S,3R,5R)-3-(2-(2- fluorophenyl)-6-methoxy-1H- imidazo[4,5-c]pyridin-1-yl)-5- hydroxycyclohexyl)thiazole-2- carboxamide |
| 278 | 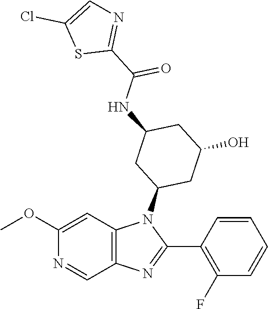 [SMILES: COc1cc2c(cn1)nc(-c1ccccc1F)n2[C@@H]1C[C@@H](O)C[C@H](NC(=O)c2ncc(Cl)s2)C1] | 5-chloro-N-((1R,3S,5S)-3-(2-(2- fluorophenyl)-6-methoxy-1H- imidazo[4,5-c]pyridin-1-yl)-5- hydroxycyclohexyl)thiazole-2- carboxamide |
| 279 | 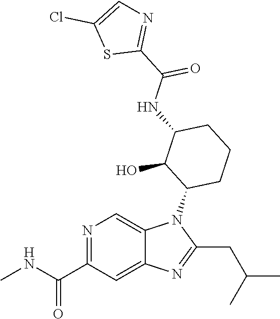 [SMILES: CNC(=O)c1cc2nc(CC(C)C)n([C@H]3CCC[C@@H](NC(=O)c4ncc(Cl)s4)[C@@H]3O)c2cn1] | 5-chloro-N-((1R,2S,3S)-2- hydroxy-3-(2-isobutyl-6- (methylcarbamoyl)-3H- imidazo[4,5-c]pyridin-3- yl)cyclohexyl)thiazole-2- carboxamide |
| 280 | 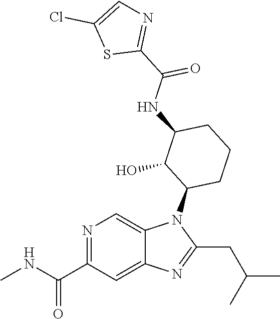 [SMILES: CNC(=O)c1cc2nc(CC(C)C)n([C@@H]3CCC[C@H](NC(=O)c4ncc(Cl)s4)[C@H]3O)c2cn1] | 5-chloro-N-((1S,2R,3R)-2- hydroxy-3-(2-isobutyl-6- (methylcarbamoyl)-3H- imidazo[4,5-c]pyridin-3- yl)cyclohexyl)thiazole-2- carboxamide |
| 281 | 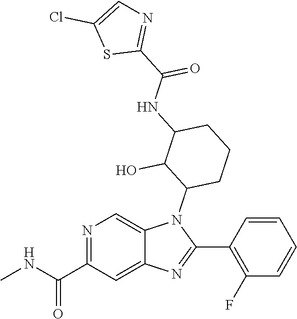 [SMILES: CNC(=O)c1cc2nc(-c3ccccc3F)n(C3CCCC(NC(=O)c4ncc(Cl)s4)C3O)c2cn1] | 5-chloro-N-(3-(2-(2- fluorophenyl)-6- (methylcarbamoyl)-3H- imidazo[4,5-c]pyridin-3-yl)-2- hydroxycyclohexyl)thiazole-2- carboxamide⁽⁸⁾ |

⁽⁸⁾relative stereochemistry at centers 1 and 3 of the cyclohexyl ring is cis with —OH at center 2 trans.

In some embodiments, the disclosure provides specific examples of Formulas IVA and IVB, and their pharmaceutically acceptable salts, tautomers, and/or isotopologues as set forth in Table 4 below.

TABLE 4

| Ex. No. | Structure | Chemical Name |
|---|---|---|
| 282 | 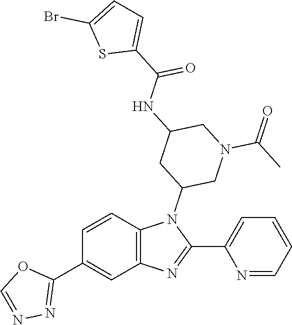 [SMILES: CC(=O)N1CC(NC(=O)c2ccc(Br)s2)CC(n2c(-c3ccccn3)nc3cc(-c4nnco4)ccc32)C1] | N-(5-(5-(1,3,4-oxadiazol-2-yl)-2- (pyridin-2-yl)-1H- benzo[d]imidazol-1-yl)-1- acetylpiperidin-3-yl)-5- bromothiophene-2-carboxamide⁽⁴⁾ |
| 283 | 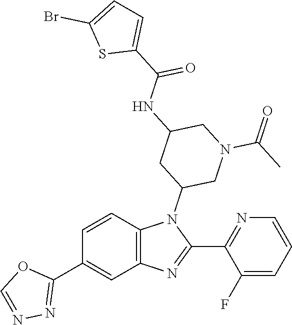 [SMILES: CC(=O)N1CC(NC(=O)c2ccc(Br)s2)CC(n2c(-c3ncccc3F)nc3cc(-c4nnco4)ccc32)C1] | N-(1-acetyl-5-(2-(3-fluoropyridin- 2-yl)-5-(1,3,4-oxadiazol-2-yl)-1H- benzo[d]imidazol-1-yl)piperidin- 3-yl)-5-bromothiophene-2- carboxamide⁽⁴⁾ |
| 284 | 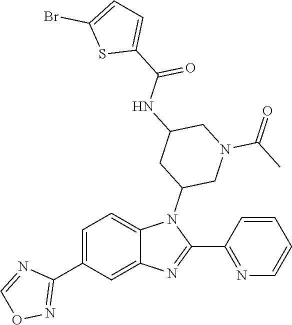 [SMILES: CC(=O)N1CC(NC(=O)c2ccc(Br)s2)CC(n2c(-c3ccccn3)nc3cc(-c4ncon4)ccc32)C1] | N-(5-(5-(1,2,4-oxadiazol-3-yl)-2- (pyridin-2-yl)-1H- benzo[d]imidazol-1-yl)-1- acetylpiperidin-3-yl)-5- bromothiophene-2-carboxamide⁽⁴⁾ |
| 285 | 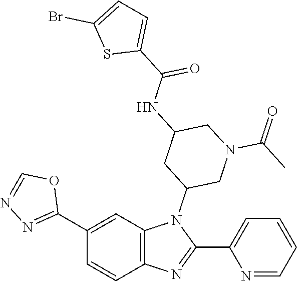 [SMILES: CC(=O)N1CC(NC(=O)c2ccc(Br)s2)CC(n2c(-c3ccccn3)nc3ccc(-c4nnco4)cc32)C1] | N-(5-(6-(1,3,4-oxadiazol-2-yl)-2- (pyridin-2-yl)-1H- benzo[d]imidazol-1-yl)-1- acetylpiperidin-3-yl)-5- bromothiophene-2-carboxamide⁽⁴⁾ |
| 286 | 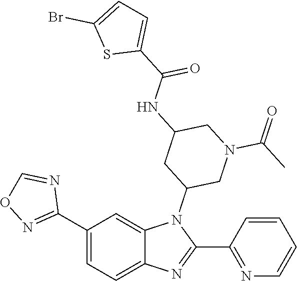 [SMILES: CC(=O)N1CC(NC(=O)c2ccc(Br)s2)CC(n2c(-c3ccccn3)nc3ccc(-c4ncon4)cc32)C1] | N-(5-(6-(1,2,4-oxadiazol-3-yl)-2- (pyridin-2-yl)-1H- benzo[d]imidazol-1-yl)-1- acetylpiperidin-3-yl)-5- bromothiophene-2-carboxamide⁽⁴⁾ |
| 287 | 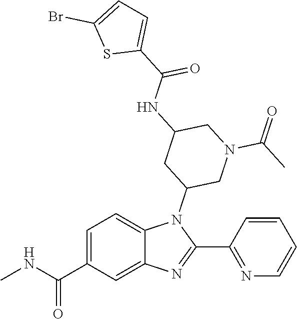 [SMILES: CNC(=O)c1ccc2c(c1)nc(-c1ccccn1)n2C1CC(NC(=O)c2ccc(Br)s2)CN(C(C)=O)C1] | 1-(1-acetyl-5-(5-bromothiophene- 2-carboxamido)piperidin-3-yl)-N- methyl-2-(pyridin-2-yl)-1H- benzo[d]imidazole-5- carboxamide⁽⁴⁾ |
| 288 | 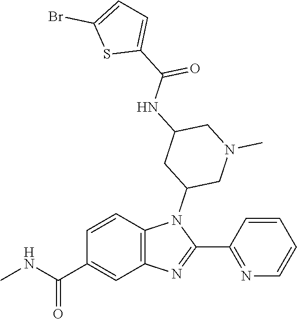 [SMILES: CNC(=O)c1ccc2c(c1)nc(-c1ccccn1)n2C1CC(NC(=O)c2ccc(Br)s2)CN(C)C1] | 1-(5-(5-bromothiophene-2- carboxamido)-1-methylpiperidin- 3-yl)-N-methyl-2-(pyridin-2-yl)- 1H-benzo[d]imidazole-5- carboxamide⁽⁴⁾ |
| 289 | 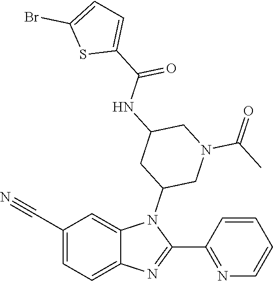 [SMILES: CC(=O)N1CC(NC(=O)c2ccc(Br)s2)CC(n2c(-c3ccccn3)nc3ccc(C#N)cc32)C1] | N-(1-acetyl-5-(6-cyano-2- (pyridin-2-yl)-1H- benzo[d]imidazol-1-yl)piperidin- 3-yl)-5-bromothiophene-2- carboxamide⁽⁴⁾ |
| 290 | 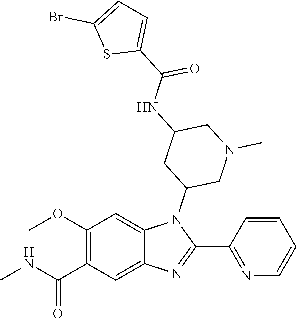 [SMILES: CNC(=O)c1cc2nc(-c3ccccn3)n(C3CC(NC(=O)c4ccc(Br)s4)CN(C)C3)c2cc1OC] | 1-(5-(5-bromothiophene-2- carboxamido)-1-methylpiperidin- 3-yl)-6-methoxy-N-methyl-2- (pyridin-2-yl)-1H- benzo[d]imidazole-5- carboxamide⁽⁴⁾ |
| 291 | 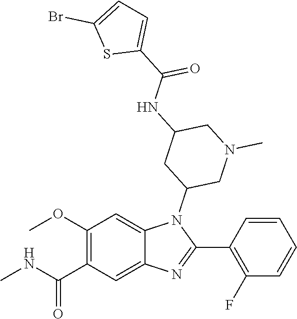 [SMILES: CNC(=O)c1cc2nc(-c3ccccc3F)n(C3CC(NC(=O)c4ccc(Br)s4)CN(C)C3)c2cc1OC] | 1-(5-(5-bromothiophene-2- carboxamido)-1-methylpiperidin- 3-yl)-2-(2-fluorophenyl)-6- methoxy-N-methyl-1H- benzo[d]imidazole-5- carboxamide⁽⁴⁾ |
| 292 | 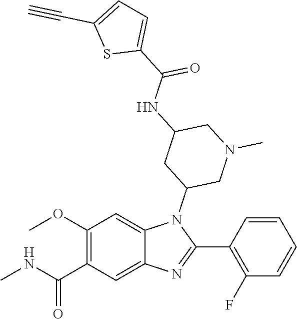 [SMILES: C#Cc1ccc(C(=O)NC2CC(n3c(-c4ccccc4F)nc4cc(C(=O)NC)c(OC)cc43)CN(C)C2)s1] | 1-(5-(5-ethynylthiophene-2- carboxamido)-1-methylpiperidin- 3-yl)-2-(2-fluorophenyl)-6- methoxy-N-methyl-1H- benzo[d]imidazole-5- carboxamide⁽⁴⁾ |
| 293 | 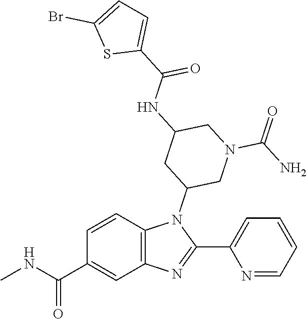 [SMILES: CNC(=O)c1ccc2c(c1)nc(-c1ccccn1)n2C1CC(NC(=O)c2ccc(Br)s2)CN(C(N)=O)C1] | 1-(5-(5-bromothiophene-2- carboxamido)-1- carbamoylpiperidin-3-yl)-N- methyl-2-(pyridin-2-yl)-1H- benzo[d]imidazole-5- carboxamide⁽⁴⁾ |

⁽⁴⁾relative stereochemistry at centers 3 and 5 of piperidine ring is cis.

In some embodiments, the disclosure provides specific examples of Formulas VA and VB, and their pharmaceutically acceptable salts, tautomers, and/or isotopologues as set forth in Table 5 below.

TABLE 5

| Ex. No. | Structure | Chemical Name |
|---|---|---|
| 294 | 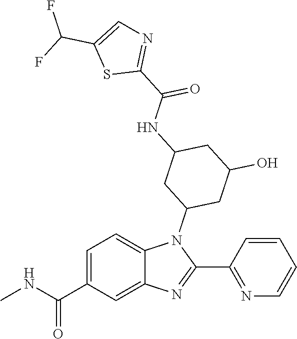 [SMILES: CNC(=O)c1ccc2c(c1)nc(-c1ccccn1)n2C1CC(O)CC(NC(=O)c2ncc(C(F)F)s2)C1] | 5-(difluoromethyl)-N-(3-hydroxy- 5-(5-(methylcarbamoyl)-2- (pyridin-2-yl)-1H- benzo[d]imidazol-1- yl)cyclohexyl)thiazole-2- carboxamide⁽⁹⁾ |
| 465 | 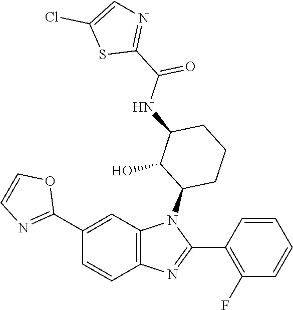 [SMILES: O=C(N[C@H]1CCC[C@@H](n2c(-c3ccccc3F)nc3ccc(-c4ncco4)cc32)[C@@H]1O)c1ncc(Cl)s1] | 5-chloro-N-((1S,2R,3R)-3-(2-(2- fluorophenyl)-6-(oxazol-2-yl)-1H- benzo[d]imidazol-1-yl)-2- hydroxycyclohexyl)thiazole-2- carboxamide |
| 466 | 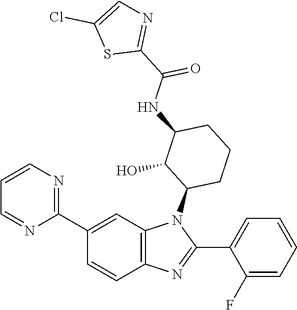 [SMILES: O=C(N[C@H]1CCC[C@@H](n2c(-c3ccccc3F)nc3ccc(-c4ncccn4)cc32)[C@@H]1O)c1ncc(Cl)s1] | 5-chloro-N-((1S,2R,3R)-3-(2-(2- fluorophenyl)-6-(pyrimidin-2-yl)- 1H-benzo[d]imidazol-1-yl)-2- hydroxycyclohexyl)thiazole-2- carboxamide |

⁽⁹⁾relative stereochemistry at centers 1 and 5 of cyclohexyl ring is cis with —OH at center 3 trans.

In further embodiments, the following Formula VA compounds (including all stereoisomers), and their pharmaceutically acceptable salts, tautomers, and/or isotopologues, can be synthesized using the Intermediate and General Schemes and Examples set forth herein, or a combination of the procedures described herein using ordinary skill in the art.

TABLE 5A
5-chloro-N-(2-hydroxy-3-(2-isobutyl-6-(oxazol-2-yl)-1H-benzo[d]imidazol-1-
yl)cyclohexyl)thiazole-2-carboxamide
5-chloro-N-(2-hydroxy-3-(2-isobutyl-6-(4-methyloxazol-2-yl)-1H-benzo[d]
imidazol-1-yl)cyclohexyl)thiazole-2-carboxamide
5-chloro-N-(2-hydroxy-3-(2-isobutyl-6-(oxazol-2-yl)-1H-benzo[d]imidazol-1-
yl)cyclohexyl)thiophene-2-carboxamide
5-chloro-N-(2-hydroxy-3-(2-isobutyl-6-(4-methyloxazol-2-yl)-1H-benzo[d]
imidazol-1-yl)cyclohexyl)thiophene-2-carboxamide
5-chloro-N-(2-hydroxy-3-(2-isobutyl-6-(pyrimidin-2-yl)-1H-benzo[d]imidazol-1-
yl)cyclohexyl)thiazole-2-carboxamide
5-chloro-N-(2-hydroxy-3-(2-isobutyl-6-(pyrimidin-2-yl)-1H-benzo[d]imidazol-1-
yl)cyclohexyl)thiophene-2-carboxamide
5-chloro-N-(2-hydroxy-3-(2-isobutyl-6-(pyridin-2-yl)-1H-benzo[d]imidazol-1-
yl)cyclohexyl)thiazole-2-carboxamide
5-chloro-N-(2-hydroxy-3-(2-isobutyl-6-(pyridin-2-yl)-1H-benzo[d]imidazol-1-
yl)cyclohexyl)thiophene-2-carboxamide
5-chloro-N-(2-hydroxy-3-(2-isobutyl-6-(1H-1,2,4-triazol-3-yl)-1H-benzo[d]
imidazol-1-yl)cyclohexyl)thiazole-2-carboxamide
5-chloro-N-(2-hydroxy-3-(2-isobutyl-6-(1H-1,2,4-triazol-3-yl)-1H-benzo[d]
imidazol-1-yl)cyclohexyl)thiophene-2-carboxamide
5-chloro-N-(2-hydroxy-3-(2-isobutyl-6-(thiazol-2-yl)-1H-benzo[d]imidazol-1-
yl)cyclohexyl)thiazole-2-carboxamide
5-chloro-N-(2-hydroxy-3-(2-isobutyl-6-(thiazol-2-yl)-1H-benzo[d]imidazol-1-
yl)cyclohexyl)thiophene-2-carboxamide
5-chloro-N-(2-hydroxy-3-(2-neopentyl-6-(oxazol-2-yl)-1H-benzo[d]imidazol-1-
yl)cyclohexyl)thiazole-2-carboxamide
5-chloro-N-(2-hydroxy-3-(2- neopentyl -6-(4-methyloxazol-2-yl)-1H-benzo[d]
imidazol-1-yl)cyclohexyl)thiazole-2-carboxamide
5-chloro-N-(2-hydroxy-3-(2- neopentyl -6-(oxazol-2-yl)-1H-benzo[d]imidazol-1-
yl)cyclohexyl)thiophene-2-carboxamide
5-chloro-N-(2-hydroxy-3-(2- neopentyl -6-(4-methyloxazol-2-yl)-1H-benzo[d]
imidazol-1-yl)cyclohexyl)thiophene-2-carboxamide
5-chloro-N-(2-hydroxy-3-(2- neopentyl -6-(pyrimidin-2-yl)-1H-benzo[d]imidazol-
1-yl)cyclohexyl)thiazole-2-carboxamide
5-chloro-N-(2-hydroxy-3-(2- neopentyl -6-(pyrimidin-2-yl)-1H-benzo[d]imidazol-
1-yl)cyclohexyl)thiophene-2-carboxamide
5-chloro-N-(2-hydroxy-3-(2- neopentyl -6-(pyridin-2-yl)-1H-benzo[d]imidazol-1-
yl)cyclohexyl)thiazole-2-carboxamide
5-chloro-N-(2-hydroxy-3-(2- neopentyl -6-(pyridin-2-yl)-1H-benzo[d]imidazol-1-
yl)cyclohexyl)thiophene-2-carboxamide
5-chloro-N-(2-hydroxy-3-(2- neopentyl -6-(1H-1,2,4-triazol-3-yl)-1H-benzo[d]
imidazol-1-yl)cyclohexyl)thiazole-2-carboxamide
5-chloro-N-(2-hydroxy-3-(2- neopentyl -6-(1H-1,2,4-triazol-3-yl)-1H-benzo[d]
imidazol-1-yl)cyclohexyl)thiophene-2-carboxamide
5-chloro-N-(2-hydroxy-3-(2- neopentyl -6-(thiazol-2-yl)-1H-benzo[d]imidazol-1-
yl)cyclohexyl)thiazole-2-carboxamide
5-chloro-N-(2-hydroxy-3-(2- neopentyl-6-(thiazol-2-yl)-1H-benzo[d]imidazol-1-
yl)cyclohexyl)thiophene-2-carboxamide
5-chloro-N-(3-(2-(2-fluorophenyl)-6-(oxazol-2-yl)-1H-benzo[d]imidazol-1-yl)-2-
hydroxycyclohexyl)thiazole-2-carboxamide
5-chloro-N-(3-(2-(2-fluorophenyl)-6-(4-methyloxazol-2-yl)-1H-benzo[d]imidazol-
1-yl)-2-hydroxycyclohexyl)thiazole-2-carboxamide
5-chloro-N-(3-(2-(2-fluorophenyl)-6-(oxazol-2-yl)-1H-benzo[d]imidazol-1-yl)-2-
hydroxycyclohexyl)thiophene-2-carboxamide
5-chloro-N-(3-(2-(2-fluorophenyl)-6-(4-methyloxazol-2-yl)-1H-benzo[d]imidazol-
1-yl)-2-hydroxycyclohexyl)thiophene-2-carboxamide
5-chloro-N-(3-(2-(2-fluorophenyl)-6-(pyrimidin-2-yl)-1H-benzo[d]imidazol-1-yl)-
2-hydroxycyclohexyl)thiazole-2-carboxamide
5-chloro-N-(3-(2-(2-fluorophenyl)-6-(pyrimidin-2-yl)-1H-benzo[d]imidazol-1-yl)-2-
hydroxycyclohexyl)thiophene-2-carboxamide
5-chloro-N-(3-(2-(2-fluorophenyl)-6-(pyridin-2-yl)-1H-benzo[d]imidazol-1-yl)-2-
hydroxycyclohexyl)thiazole-2-carboxamide
5-chloro-N-(3-(2-(2-fluorophenyl)-6-(pyridin-2-yl)-1H-benzo[d]imidazol-1-yl)-2-
hydroxycyclohexyl)thiophene-2-carboxamide
5-chloro-N-(3-(2-(2-fluorophenyl)-6-(1H-1,2,4-triazol-3-yl)-1H-benzo[d]imidazol-1-yl)-2-
hydroxycyclohexyl)thiazole-2-carboxamide
5-chloro-N-(3-(2-(2-fluorophenyl)-6-(1H-1,2,4-triazol-3-yl)-1H-benzo[d]imidazol-1-yl)-2-
hydroxycyclohexyl)thiophene-2-carboxamide
5-chloro-N-(3-(2-(2-fluorophenyl)-6-(thiazol-2-yl)-1H-benzo[d]imidazol-1-yl)-2-
hydroxycyclohexyl)thiazole-2-carboxamide
5-chloro-N-(3-(2-(2-fluorophenyl)-6-(thiazol-2-yl)-1H-benzo[d]imidazol-1-yl)-2-
hydroxycyclohexyl)thiophene-2-carboxamide
5-chloro-N-(3-(2-(2-fluoro-6-hydroxyphenyl)-6-(oxazol-2-yl)-1H-benzo[d]imidazol-1-yl)-
2-hydroxycyclohexyl)thiazole-2-carboxamide
5-chloro-N-(3-(2-(2-fluoro-6-hydroxyphenyl)-6-(4-methyloxazol-2-yl)-1H-
benzo[d]imidazol-1-yl)-2-hydroxycyclohexyl)thiazole-2-carboxamide
5-chloro-N-(3-(2-(2-fluoro-6-hydroxyphenyl)-6-(oxazol-2-yl)-1H-benzo[d]imidazol-1-yl)-
2-hydroxycyclohexyl)thiophene-2-carboxamide
5-chloro-N-(3-(2-(2-fluoro-6-hydroxyphenyl)-6-(4-methyloxazol-2-yl)-1H-
benzo[d]imidazol-1-yl)-2-hydroxycyclohexyl)thiophene-2-carboxamide
5-chloro-N-(3-(2-(2-fluoro-6-hydroxyphenyl)-6-(pyrimidin-2-yl)-1H-benzo[d]imidazol-1-
yl)-2-hydroxycyclohexyl)thiazole-2-carboxamide
5-chloro-N-(3-(2-(2-fluoro-6-hydroxyphenyl)-6-(pyrimidin-2-yl)-1H-benzo[d]imidazol-1-
yl)-2-hydroxycyclohexyl)thiophene-2-carboxamide
5-chloro-N-(3-(2-(2-fluoro-6-hydroxyphenyl)-6-(pyridin-2-yl)-1H-benzo[d]imidazol-1-yl)-
2-hydroxycyclohexyl)thiazole-2-carboxamide
5-chloro-N-(3-(2-(2-fluoro-6-hydroxyphenyl)-6-(pyridin-2-yl)-1H-benzo[d]imidazol-1-yl)-
2-hydroxycyclohexyl)thiophene-2-carboxamide
5-chloro-N-(3-(2-(2-fluoro-6-hydroxyphenyl)-6-(1H-1,2,4-triazol-3-yl)-1H-
benzo[d]imidazol-1-yl)-2-hydroxycyclohexyl)thiazole-2-carboxamide
5-chloro-N-(3-(2-(2-fluoro-6-hydroxyphenyl)-6-(1H-1,2,4-triazol-3-yl)-1H-
benzo[d]imidazol-1-yl)-2-hydroxycyclohexyl)thiophene-2-carboxamide
5-chloro-N-(3-(2-(2-fluoro-6-hydroxyphenyl)-6-(thiazol-2-yl)-1H-benzo[d]imidazol-1-yl)-
2-hydroxycyclohexyl)thiazole-2-carboxamide
5-chloro-N-(3-(2-(2-fluoro-6-hydroxyphenyl)-6-(thiazol-2-yl)-1H-benzo[d]imidazol-1-yl)-
2-hydroxycyclohexyl)thiophene-2-carboxamide

In some embodiments, the compounds described in Table 5A are the (1 S,2R,3R) stereoisomer, or their pharmaceutically acceptable salts, tautomers, and/or isotopologues.

5-chloro-N-((1S,2R,3R)-2-hydroxy-3-(2-isobutyl-6-(oxazol-2-yl)-1H-benzo[d]imidazol-1-
yl)cyclohexyl)thiazole-2-carboxamide
5-chloro-N-((1S,2R,3R)-2-hydroxy-3-(2-isobutyl-6-(4-methyloxazol-2-yl)-1H-
benzo[d]imidazol-1-yl)cyclohexyl)thiazole-2-carboxamide
5-chloro-N-((1S,2R,3R)-2-hydroxy-3-(2-isobutyl-6-(oxazol-2-yl)-1H-benzo[d]imidazol-1-
yl)cyclohexyl)thiophene-2-carboxamide
5-chloro-N-((1S,2R,3R)-2-hydroxy-3-(2-isobutyl-6-(4-methyloxazol-2-yl)-1H-
benzo[d]imidazol-1-yl)cyclohexyl)thiophene-2-carboxamide
5-chloro-N-((1S,2R,3R)-2-hydroxy-3-(2-isobutyl-6-(pyrimidin-2-yl)-1H-
benzo[d]imidazol-1-yl)cyclohexyl)thiazole-2-carboxamide
5-chloro-N-((1S,2R,3R)-2-hydroxy-3-(2-isobutyl-6-(pyrimidin-2-yl)-1H-
benzo[d]imidazol-1-yl)cyclohexyl)thiophene-2-carboxamide
5-chloro-N-((1S,2R,3R)-2-hydroxy-3-(2-isobutyl-6-(pyridin-2-yl)-1H-benzo[d]imidazol-1-
yl)cyclohexyl)thiazole-2-carboxamide
5-chloro-N-((1S,2R,3R)-2-hydroxy-3-(2-isobutyl-6-(pyridin-2-yl)-1H-benzo[d]imidazol-1-
yl)cyclohexyl)thiophene-2-carboxamide
5-chloro-N-((1S,2R,3R)-2-hydroxy-3-(2-isobutyl-6-(1H-1,2,4-triazol-3-yl)-1H-
benzo[d]imidazol-1-yl)cyclohexyl)thiazole-2-carboxamide
5-chloro-N-((1S,2R,3R)-2-hydroxy-3-(2-isobutyl-6-(1H-1,2,4-triazol-3-yl)-1H-
benzo[d]imidazol-1-yl)cyclohexyl)thiophene-2-carboxamide
5-chloro-N-((1S,2R,3R)-2-hydroxy-3-(2-isobutyl-6-(thiazol-2-yl)-1H-benzo[d]imidazol-1-
yl)cyclohexyl)thiazole-2-carboxamide
5-chloro-N-((1S,2R,3R)-2-hydroxy-3-(2-isobutyl-6-(thiazol-2-yl)-1H-benzo[d]imidazol-1-
yl)cyclohexyl)thiophene-2-carboxamide
5-chloro-N-((1S,2R,3R)-2-hydroxy-3-(2-neopentyl-6-(oxazol-2-yl)-1H-benzo[d]imidazol-
1-yl)cyclohexyl)thiazole-2-carboxamide
5-chloro-N-((1S,2R,3R)-2-hydroxy-3-(2- neopentyl -6-(4-methyloxazol-2-yl)-1H-
benzo[d]imidazol-1-yl)cyclohexyl)thiazole-2-carboxamide
5-chloro-N-((1S,2R,3R)-2-hydroxy-3-(2- neopentyl -6-(oxazol-2-yl)-1H-benzo[d]imidazol-
1-yl)cyclohexyl)thiophene-2-carboxamide
5-chloro-N-((1S,2R,3R)-2-hydroxy-3-(2- neopentyl -6-(4-methyloxazol-2-yl)-1H-
benzo[d]imidazol-1-yl)cyclohexyl)thiophene-2-carboxamide
5-chloro-N-((1S,2R,3R)-2-hydroxy-3-(2- neopentyl -6-(pyrimidin-2-yl)-1H-
benzo[d]imidazol-1-yl)cyclohexyl)thiazole-2-carboxamide
5-chloro-N-((1S,2R,3R)-2-hydroxy-3-(2- neopentyl -6-(pyrimidin-2-yl)-1H-
benzo[d]imidazol-1-yl)cyclohexyl)thiophene-2-carboxamide
5-chloro-N-((1S,2R,3R)-2-hydroxy-3-(2- neopentyl -6-(pyridin-2-yl)-1H-
benzo[d]imidazol-1-yl)cyclohexyl)thiazole-2-carboxamide
5-chloro-N-((1S,2R,3R)-2-hydroxy-3-(2- neopentyl -6-(pyridin-2-yl)-1H-
benzo[d]imidazol-1-yl)cyclohexyl)thiophene-2-carboxamide
5-chloro-N-((1S,2R,3R)-2-hydroxy-3-(2- neopentyl -6-(1H-1,2,4-triazol-3-yl)-1H-
benzo[d]imidazol-1-yl)cyclohexyl)thiazole-2-carboxamide
5-chloro-N-((1S,2R,3R)-2-hydroxy-3-(2- neopentyl -6-(1H-1,2,4-triazol-3-yl)-1H-
benzo[d]imidazol-1-yl)cyclohexyl)thiophene-2-carboxamide
5-chloro-N-((1S,2R,3R)-2-hydroxy-3-(2- neopentyl -6-(thiazol-2-yl)-1H-
benzo[d]imidazol-1-yl)cyclohexyl)thiazole-2-carboxamide
5-chloro-N-((1S,2R,3R)-2-hydroxy-3-(2- neopentyl -6-(thiazol-2-yl)-1H-
benzo[d]imidazol-1-yl)cyclohexyl)thiophene-2-carboxamide
5-chloro-N-((1S,2R,3R)-3-(2-(2-fluorophenyl)-6-(4-methyloxazol-2-yl)-1H-
benzo[d]imidazol-1-yl)-2-hydroxycyclohexyl)thiazole-2-carboxamide
5-chloro-N-((1S,2R,3R)-3-(2-(2-fluorophenyl)-6-(oxazol-2-yl)-1H-benzo[d]imidazol-1-yl)-
2-hydroxycyclohexyl)thiophene-2-carboxamide
5-chloro-N-((1S,2R,3R)-3-(2-(2-fluorophenyl)-6-(4-methyloxazol-2-yl)-1H-
benzo[d]imidazol-1-yl)-2-hydroxycyclohexyl)thiophene-2-carboxamide
5-chloro-N-((1S,2R,3R)-3-(2-(2-fluorophenyl)-6-(pyrimidin-2-yl)-1H-benzo[d]imidazol-1-
yl)-2-hydroxycyclohexyl)thiophene-2-carboxamide
5-chloro-N-((1S,2R,3R)-3-(2-(2-fluorophenyl)-6-(pyridin-2-yl)-1H-benzo[d]imidazol-1-
yl)-2-hydroxycyclohexyl)thiazole-2-carboxamide
5-chloro-N-((1S,2R,3R)-3-(2-(2-fluorophenyl)-6-(pyridin-2-yl)-1H-benzo[d]imidazol-1-
yl)-2-hydroxycyclohexyl)thiophene-2-carboxamide
5-chloro-N-((1S,2R,3R)-3-(2-(2-fluorophenyl)-6-(1H-1,2,4-triazol-3-yl)-1H-
benzo[d]imidazol-1-yl)-2-hydroxycyclohexyl)thiazole-2-carboxamide
5-chloro-N-((1S,2R,3R)-3-(2-(2-fluorophenyl)-6-(1H-1,2,4-triazol-3-yl)-1H-
benzo[d]imidazol-1-yl)-2-hydroxycyclohexyl)thiophene-2-carboxamide
5-chloro-N-((1S,2R,3R)-3-(2-(2-fluorophenyl)-6-(thiazol-2-yl)-1H-benzo[d]imidazol-1-
yl)-2-hydroxycyclohexyl)thiazole-2-carboxamide
5-chloro-N-((1S,2R,3R)-3-(2-(2-fluorophenyl)-6-(thiazol-2-yl)-1H-benzo[d]imidazol-1-
yl)-2-hydroxycyclohexyl)thiophene-2-carboxamide
5-chloro-N-((1S,2R,3R)-3-(2-(2-fluoro-6-hydroxyphenyl)-6-(oxazol-2-yl)-1H-
benzo[d]imidazol-1-yl)-2-hydroxycyclohexyl)thiazole-2-carboxamide
5-chloro-N-((1S,2R,3R)-3-(2-fluoro-6-hydroxyphenyl)-6-(4-methyloxazol-2-yl)-1H-
benzo[d]imidazol-1-yl)-2-hydroxycyclohexyl)thiazole-2-carboxamide
5-chloro-N-((1S,2R,3R)-3-(2-(2-fluoro-6-hydroxyphenyl)-6-(oxazol-2-yl)-1H-
benzo[d]imidazol-1-yl)-2-hydroxycyclohexyl)thiophene-2-carboxamide
5-chloro-N-((1S,2R,3R)-3-(2-(2-fluoro-6-hydroxyphenyl)-6-(4-methyloxazol-2-yl)-1H-
benzo[d]imidazol-1-yl)-2-hydroxycyclohexyl)thiophene-2-carboxamide
5-chloro-N-((1S,2R,3R)-3-(2-(2-fluoro-6-hydroxyphenyl)-6-(pyrimidin-2-yl)-1H-
benzo[d]imidazol-1-yl)-2-hydroxycyclohexyl)thiazole-2-carboxamide
5-chloro-N-((1S,2R,3R)-3-(2-(2-fluoro-6-hydroxyphenyl)-6-(pyrimidin-2-yl)-1H-
benzo[d]imidazol-1-yl)-2-hydroxycyclohexyl)thiophene-2-carboxamide
5-chloro-N-((1S,2R,3R)-3-(2-(2-fluoro-6-hydroxyphenyl)-6-(pyridin-2-yl)-1H-
benzo[d]imidazol-1-yl)-2-hydroxycyclohexyl)thiazole-2-carboxamide
5-chloro-N-((1S,2R,3R)-3-(2-(2-fluoro-6-hydroxyphenyl)-6-(pyridin-2-yl)-1H-
benzo[d]imidazol-1-yl)-2-hydroxycyclohexyl)thiophene-2-carboxamide
5-chloro-N-((1S,2R,3R)-3-(2-(2-fluoro-6-hydroxyphenyl)-6-(1H-1,2,4-triazol-3-yl)-1H-
benzo[d]imidazol-1-yl)-2-hydroxycyclohexyl)thiazole-2-carboxamide
5-chloro-N-((1S,2R,3R)-3-(2-(2-fluoro-6-hydroxyphenyl)-6-(1H-1,2,4-triazol-3-yl)-1H-
benzo[d]imidazol-1-yl)-2-hydroxycyclohexyl)thiophene-2-carboxamide
5-chloro-N-((1S,2R,3R)-3-(2-(2-fluoro-6-hydroxyphenyl)-6-(thiazol-2-yl)-1H-
benzo[d]imidazol-1-yl)-2-hydroxycyclohexyl)thiazole-2-carboxamide
5-chloro-N-((1S,2R,3R)-3-(2-(2-fluoro-6-hydroxyphenyl)-6-(thiazol-2-yl)-1H-
benzo[d]imidazol-1-yl)-2-hydroxycyclohexyl)thiophene-2-carboxamide

In other embodiments, the compounds described in Table 5A are the (1 S,2S,3R) stereoisomer, or their pharmaceutically acceptable salt, tautomers, and/or isotopologues.

5-chloro-N-((1S,2S,3R)-2-hydroxy-3-(2-isobutyl-6-(oxazol-2-yl)-1H-
benzo[d]imidazol-1-yl)cyclohexyl)thiazole-2-carboxamide
5-chloro-N-((1S,2S,3R)-2-hydroxy-3-(2-isobutyl-6-(4-methyloxazol-2-yl)-1H-
benzo[d]imidazol-1-yl)cyclohexyl)thiazole-2-carboxamide
5-chloro-N-((1S,2S,3R)-2-hydroxy-3-(2-isobutyl-6-(oxazol-2-yl)-1H-
benzo[d]imidazol-1-yl)cyclohexyl)thiophene-2-carboxamide
5-chloro-N-((1S,2S,3R)-2-hydroxy-3-(2-isobutyl-6-(4-methyloxazol-2-yl)-1H-
benzo[d]imidazol-1-yl)cyclohexyl)thiophene-2-carboxamide
5-chloro-N-((1S,2S,3R)-2-hydroxy-3-(2-isobutyl-6-(pyrimidin-2-yl)-1H-
benzo[d]imidazol-1-yl)cyclohexyl)thiazole-2-carboxamide
5-chloro-N-((1S,2S,3R)-2-hydroxy-3-(2-isobutyl-6-(pyrimidin-2-yl)-1H-
benzo[d]imidazol-1-yl)cyclohexyl)thiophene-2-carboxamide
5-chloro-N-((1S,2S,3R)-2-hydroxy-3-(2-isobutyl-6-(pyridin-2-yl)-1H-
benzo[d]imidazol-1-yl)cyclohexyl)thiazole-2-carboxamide
5-chloro-N-((1S,2S,3R)-2-hydroxy-3-(2-isobutyl-6-(pyridin-2-yl)-1H-
benzo[d]imidazol-1-yl)cyclohexyl)thiophene-2-carboxamide
5-chloro-N-((1S,2S,3R)-2-hydroxy-3-(2-isobutyl-6-(1H-1,2,4-triazol-3-yl)-1H-
benzo[d]imidazol-1-yl)cyclohexyl)thiazole-2-carboxamide
5-chloro-N-((1S,2S,3R)-2-hydroxy-3-(2-isobutyl-6-(1H-1,2,4-triazol-3-yl)-1H-
benzo[d]imidazol-1-yl)cyclohexyl)thiophene-2-carboxamide
5-chloro-N-((1S,2S,3R)-2-hydroxy-3-(2-isobutyl-6-(thiazol-2-yl)-1H-
benzo[d]imidazol-1-yl)cyclohexyl)thiazole-2-carboxamide
5-chloro-N-((1S,2S,3R)-2-hydroxy-3-(2-isobutyl-6-(thiazol-2-yl)-1H-
benzo[d]imidazol-1-yl)cyclohexyl)thiophene-2-carboxamide
5-chloro-N-((1S,2S,3R)-2-hydroxy-3-(2- neopentyl -6-(oxazol-2-yl)-1H-
benzo[d]imidazol-1-yl)cyclohexyl)thiazole-2-carboxamide
5-chloro-N-((1S,2S,3R)-2-hydroxy-3-(2- neopentyl -6-(4-methyloxazol-2-yl)-1H-
benzo[d]imidazol-1-yl)cyclohexyl)thiazole-2-carboxamide
5-chloro-N-((1S,2S,3R)-2-hydroxy-3-(2- neopentyl -6-(oxazol-2-yl)-1H-
benzo[d]imidazol-1-yl)cyclohexyl)thiophene-2-carboxamide
5-chloro-N-((1S,2S,3R)-2-hydroxy-3-(2- neopentyl -6-(4-methyloxazol-2-yl)-1H-
benzo[d]imidazol-1-yl)cyclohexyl)thiophene-2-carboxamide
5-chloro-N-((1S,2S,3R)-2-hydroxy-3-(2- neopentyl -6-(pyrimidin-2-yl)-1H-
benzo[d]imidazol-1-yl)cyclohexyl)thiazole-2-carboxamide
5-chloro-N-((1S,2S,3R)-2-hydroxy-3-(2- neopentyl -6-(pyrimidin-2-yl)-1H-
benzo[d]imidazol-1-yl)cyclohexyl)thiophene-2-carboxamide
5-chloro-N-((1S,2S,3R)-2-hydroxy-3-(2- neopentyl -6-(pyridin-2-yl)-1H-
benzo[d]imidazol-1-yl)cyclohexyl)thiazole-2-carboxamide
5-chloro-N-((1S,2S,3R)-2-hydroxy-3-(2- neopentyl -6-(pyridin-2-yl)-1H-
benzo[d]imidazol-1-yl)cyclohexyl)thiophene-2-carboxamide
5-chloro-N-((1S,2S,3R)-2-hydroxy-3-(2- neopentyl -6-(1H-1,2,4-triazol-3-yl)-1H-
benzo[d]imidazol-1-yl)cyclohexyl)thiazole-2-carboxamide
5-chloro-N-((1S,2S,3R)-2-hydroxy-3-(2- neopentyl -6-(1H-1,2,4-triazol-3-yl)-1H-
benzo[d]imidazol-1-yl)cyclohexyl)thiophene-2-carboxamide
5-chloro-N-((1S,2S,3R)-2-hydroxy-3-(2- neopentyl -6-(thiazol-2-yl)-1H-
benzo[d]imidazol-1-yl)cyclohexyl)thiazole-2-carboxamide
5-chloro-N-((1S,2S,3R)-2-hydroxy-3-(2- neopentyl -6-(thiazol-2-yl)-1H-
benzo[d]imidazol-1-yl)cyclohexyl)thiophene-2-carboxamide
5-chloro-N-((1S,2S,3R)-3-(2-(2-fluorophenyl)-6-(oxazol-2-yl)-1H-
benzo[d]imidazol-1-yl)-2-hydroxycyclohexyl)thiazole-2-carboxamide
5-chloro-N-((1S,2S,3R)-3-(2-(2-fluorophenyl)-6-(4-methyloxazol-2-yl)-1H-
benzo[d]imidazol-1-yl)-2-hydroxycyclohexyl)thiazole-2-carboxamide
5-chloro-N-((1S,2S,3R)-3-(2-(2-fluorophenyl)-6-(oxazol-2-yl)-1H-
benzo[d]imidazol-1-yl)-2-hydroxycyclohexyl)thiophene-2-carboxamide
5-chloro-N-((1S,2S,3R)-3-(2-(2-fluorophenyl)-6-(4-methyloxazol-2-yl)-1H-
benzo[d]imidazol-1-yl)-2-hydroxycyclohexyl)thiophene-2-carboxamide
5-chloro-N-((1S,2S,3R)-3-(2-(2-fluorophenyl)-6-(pyrimidin-2-yl)-1H-
benzo[d]imidazol-1-yl)-2-hydroxycyclohexyl)thiazole-2-carboxamide
5-chloro-N-((1S,2S,3R)-3-(2-(2-fluorophenyl)-6-(pyrimidin-2-yl)-1H-
benzo[d]imidazol-1-yl)-2-hydroxycyclohexyl)thiophene-2-carboxamide
5-chloro-N-((1S,2S,3R)-3-(2-(2-fluorophenyl)-6-(pyridin-2-yl)-1H-
benzo[d]imidazol-1-yl)-2-hydroxycyclohexyl)thiazole-2-carboxamide
5-chloro-N-((1S,2S,3R)-3-(2-(2-fluorophenyl)-6-(pyridin-2-yl)-1H-
benzo[d]imidazol-1-yl)-2-hydroxycyclohexyl)thiophene-2-carboxamide
5-chloro-N-((1S,2S,3R)-3-(2-(2-fluorophenyl)-6-(1H-1,2,4-triazol-3-yl)-1H-
benzo[d]imidazol-1-yl)-2-hydroxycyclohexyl)thiazole-2-carboxamide
5-chloro-N-((1S,2S,3R)-3-(2-(2-fluorophenyl)-6-(1H-1,2,4-triazol-3-yl)-1H-
benzo[d]imidazol-1-yl)-2-hydroxycyclohexyl)thiophene-2-carboxamide
5-chloro-N-((1S,2S,3R)-3-(2-(2-fluorophenyl)-6-(thiazol-2-yl)-1H-
benzo[d]imidazol-1-yl)-2-hydroxycyclohexyl)thiazole-2-carboxamide
5-chloro-N-((1S,2S,3R)-3-(2-(2-fluorophenyl)-6-(thiazol-2-yl)-1H-
benzo[d]imidazol-1-yl)-2-hydroxycyclohexyl)thiophene-2-carboxamide
5-chloro-N-((1S,2S,3R)-3-(2-(2-fluoro-6-hydroxyphenyl)-6-(oxazol-2-yl)-1H-
benzo[d]imidazol-1-yl)-2-hydroxycyclohexyl)thiazole-2-carboxamide
5-chloro-N-((1S,2S,3R)-3-(2-(2-fluoro-6-hydroxyphenyl)-6-(4-methyloxazol-2-yl)-1H-
benzo[d]imidazol-1-yl)-2-hydroxycyclohexyl)thiazole-2-carboxamide
5-chloro-N-((1S,2S,3R)-3-(2-(2-fluoro-6-hydroxyphenyl)-6-(oxazol-2-yl)-1H-
benzo[d]imidazol-1-yl)-2-hydroxycyclohexyl)thiophene-2-carboxamide
5-chloro-N-((1S,2S,3R)-3-(2-(2-fluoro-6-hydroxyphenyl)-6-(4-methyloxazol-2-yl)-1H-
benzo[d]imidazol-1-yl)-2-hydroxycyclohexyl)thiophene-2-carboxamide
5-chloro-N-((1S,2S,3R)-3-(2-(2-fluoro-6-hydroxyphenyl)-6-(pyrimidin-2-yl)-1H-
benzo[d]imidazol-1-yl)-2-hydroxycyclohexyl)thiazole-2-carboxamide
5-chloro-N-((1S,2S,3R)-3-(2-(2-fluoro-6-hydroxyphenyl)-6-(pyrimidin-2-yl)-1H-
benzo[d]imidazol-1-yl)-2-hydroxycyclohexyl)thiophene-2-carboxamide
5-chloro-N-((1S,2S,3R)-3-(2-(2-fluoro-6-hydroxyphenyl)-6-(pyridin-2-yl)-1H-
benzo[d]imidazol-1-yl)-2-hydroxycyclohexyl)thiazole-2-carboxamide
5-chloro-N-((1S,2S,3R)-3-(2-(2-fluoro-6-hydroxyphenyl)-6-(pyridin-2-yl)-1H-
benzo[d]imidazol-1-yl)-2-hydroxycyclohexyl)thiophene-2-carboxamide
5-chloro-N-((1S,2S,3R)-3-(2-(2-fluoro-6-hydroxyphenyl)-6-(1H-1,2,4-triazol-3-yl)-1H-
benzo[d]imidazol-1-yl)-2-hydroxycyclohexyl)thiazole-2-carboxamide
5-chloro-N-((1S,2S,3R)-3-(2-(2-fluoro-6-hydroxyphenyl)-6-(1H-1,2,4-triazol-3-yl)-1H-
benzo[d]imidazol-1-yl)-2-hydroxycyclohexyl)thiophene-2-carboxamide
5-chloro-N-((1S,2S,3R)-3-(2-(2-fluoro-6-hydroxyphenyl)-6-(thiazol-2-yl)-1H-
benzo[d]imidazol-1-yl)-2-hydroxycyclohexyl)thiazole-2-carboxamide
5-chloro-N-((1S,2S,3R)-3-(2-(2-fluoro-6-hydroxyphenyl)-6-(thiazol-2-yl)-1H-
benzo[d]imidazol-1-yl)-2-hydroxycyclohexyl)thiophene-2-carboxamide

### Pharmaceutical Compositions

The disclosure is also directed to pharmaceutical compositions comprising a compound of the disclosure, or pharmaceutically acceptable salt, tautomer, and/or isotopologue thereof. The pharmaceutical compositions are typically formulated to provide a therapeutically effective amount of a compound of the disclosure as the active ingredient, or a pharmaceutically acceptable salt, tautomer, isotopologue, ester, prodrug, or derivative thereof. In some embodiments, the pharmaceutical compositions contain a compound of the disclosure or a pharmaceutically acceptable salt thereof, and one or more pharmaceutically acceptable excipients, carriers (including inert solid diluents and fillers), diluents (including sterile aqueous solutions and various organic solvents), permeation enhancers, solubilizers and adjuvants.

As used herein, the term “active ingredient” refers to a component within a pharmaceutical composition that is responsible for the physiological or pharmacological action of the pharmaceutical composition.

The pharmaceutical compositions can be administered alone or in combination with one or more other therapeutic agents, which are also typically administered in the form of pharmaceutical compositions. Where desired, the one or more compounds of the disclosure and other therapeutic agent(s) may be mixed into a preparation or both components may be formulated into separate preparations to use them in combination separately or at the same time.

In some embodiments, the compounds disclosed herein are effective over a wide dosage range. For example, in the treatment of adult humans, dosage forms containing from about 0.01 to 1000 mg of a compound disclosed herein per day are examples of dosage forms that may be used. The exact dosage will depend upon the route of administration, the form in which the compound is administered, the subject to be treated, the body weight of the subject to be treated, and the preference and experience of the attending physician.

Unless otherwise noted, the amounts of the compounds described herein are set forth on a free base basis. That is, the amounts indicate that amount of the compound administered, exclusive of, for example, solvent or counterions (such as in pharmaceutically acceptable salts).

Described below are non-limiting exemplary pharmaceutical compositions and methods for preparing the same.

### Pharmaceutical Compositions for Oral Administration.

In some embodiments, the disclosure provides a pharmaceutical composition for oral administration containing a compound of the disclosure and pharmaceutical excipients suitable for oral administration.

In some embodiments, the disclosure provides a solid pharmaceutical composition for oral administration containing: (i) an effective amount of a compound of the disclosure; optionally (ii) an effective amount of a second therapeutic agent; and (iii) a pharmaceutical excipient suitable for oral administration. In some embodiments, the composition further contains: (iv) an effective amount of a third therapeutic agent.

In some embodiments, the pharmaceutical composition may be a pharmaceutical composition suitable for oral consumption. Pharmaceutical compositions containing a compound of the disclosure suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the compound of the disclosure into association with the carrier, which constitutes one or more necessary ingredients. In general, the compositions are prepared by uniformly and intimately admixing the compound of the disclosure with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surfactant or dispersing agent. Molded tablets can be made by molding in a suitable machine a mixture of the powdered compound moistened with an inert liquid diluent.

This disclosure further encompasses anhydrous pharmaceutical compositions and dosage forms comprising a compound of the disclosure, since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms containing a compound of the disclosure can be prepared using anhydrous or low moisture containing ingredients and low moisture or low humidity conditions. Pharmaceutical compositions and dosage forms containing a compound of the disclosure which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

The compound of the disclosure can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the disclosure to provide tablets that disintegrate when exposed to an aqueous environment. Too much of a disintegrant may produce tablets which may disintegrate in the bottle. Too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the disclosure include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants which can be used to form pharmaceutical compositions and dosage forms of the disclosure include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel, a coagulated aerosol of synthetic silica, and mixtures thereof. A lubricant can optionally be added, in an amount of less than about 1 weight percent of the pharmaceutical composition.

When aqueous suspensions and/or elixirs are desired for oral administration, the active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactants which can be used to form pharmaceutical compositions and dosage forms of the disclosure include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions.

Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof, carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but are not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof, polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10-oleate, Tween 40, Tween 60, sucrose monostearate, sucrose mono laurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In one embodiment, the composition may include a solubilizer to ensure good solubilization and/or dissolution of the compound of the disclosure and to minimize precipitation of the compound of the disclosure. This can be important for compositions for non-oral use, e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, F-caprolactone and isomers thereof, 6-valerolactone and isomers thereof, β-butyrolactone and isomers thereof, and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a subject using conventional techniques, such as distillation or evaporation. Thus, if present, the solubilizer can be in a weight ratio of less than about 10%, less than about 25%, less than about 50%, about 100%, or up to less than about 200% by weight, based on the combined weight of the drug and other excipients. If desired, very small amounts of solubilizer may also be used, such as less than about 5%, less than about 2%, less than about 1% or even less. Typically, the solubilizer may be present in an amount of less than about 1% to about 100%, more typically less than about 5% to less than about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

### Pharmaceutical Compositions for Injection.

In some embodiments, the disclosure provides a pharmaceutical composition for injection containing a compound described herein and pharmaceutical excipients suitable for injection. Components and amounts of agents in the compositions are as described herein.

The forms in which the compositions of the disclosure may be incorporated for administration by injection include aqueous or oil suspensions or emulsions. Such compositions may comprise sesame oil, corn oil, cottonseed oil, peanut oil, elixirs containing mannitol or dextrose, sterile water, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the compound of the disclosure in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

### Other Pharmaceutical Compositions.

Pharmaceutical compositions may also be prepared from compositions described herein and one or more pharmaceutically acceptable excipients suitable for topical, sublingual, buccal, rectal, intraosseous, intraocular, intranasal, epidural, or intraspinal administration. Preparations for such pharmaceutical compositions are well-known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remington's Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference herein in their entirety.

Administration of the compounds or pharmaceutical compositions of the disclosure can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g., transdermal application), rectal administration, via local delivery by catheter or stent, or through inhalation. Compounds can also be administered intraadiposally or intrathecally.

The amount of the compound administered will be dependent on the subject being treated, the severity of the disorder or condition, the rate of administration, the disposition of the compound and the discretion of the prescribing physician. However, an effective dosage is in the range of about 0.001 to about 100 mg per kg body weight per day, in single or divided doses. For a 70 kg human, this would amount to about 0.05 to 7 g/day, or. In some instances, dosage levels below the lower limit of the aforesaid range may be more than adequate, while in other cases still larger doses may be employed without causing any harmful side effect, e.g., by dividing such larger doses into several small doses for administration throughout the day.

In some embodiments, a compound of the disclosure is administered in a single dose.

Typically, such administration will be by a solid oral dosage form such as tablet or capsule. However, other routes may be used as appropriate. A single dose of a compound may also be used for treatment of an acute condition.

In some embodiments, a compound of the disclosure may be administered in multiple doses. Dosing may be about once, twice, three times, four times, five times, six times, or more than six times per day. In some embodiments, dosing may be about once a month, once every two weeks, once a week, or once every other day. In another embodiment, a compound described herein and another therapeutic agent are administered together about once per day to about 6 times per day. In another embodiment, the administration of a compound described herein and another therapeutic agent continues for less than about 7 days. In yet another embodiment the administration continues for more than about 6, 10, 14, 28 days, two months, six months, or one year. In some cases, continuous dosing is achieved and maintained as long as necessary.

Administration of the compounds disclosed herein may continue as long as necessary. In some embodiments, a compound is administered for more than 1, 2, 3, 4, 5, 6, 7, 14, or 28 days. In some embodiments, a compound is administered for less than 28, 14, 7, 6, 5, 4, 3, 2, or 1 day. In some embodiments, a compound is administered chronically on an ongoing basis, e.g., for the treatment of chronic effects. The compounds of the disclosure may be administered according to specific dosing protocols.

An effective amount of a compound of the disclosure may be administered in either single or multiple doses by any of the accepted modes of administration of therapeutic agents having similar utilities, including rectal, buccal, intranasal and transdermal routes, by intra-arterial injection, intravenously, intraperitoneally, parenterally, intramuscularly, subcutaneously, orally, topically, or as an inhalant.

The subject pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound disclosed herein as an active ingredient. In addition, it may include other medicinal or pharmaceutical agents, carriers, adjuvants, etc.

Exemplary parenteral administration forms include solutions or suspensions of active compound in sterile aqueous solutions, for example, aqueous propylene glycol or dextrose solutions. Such dosage forms can be suitably buffered, if desired.

### Methods of Use

The methods disclosed herein typically comprise administering to a subject a therapeutically effective amount of a compound disclosed herein. The therapeutically effective amount of a compound of the disclosure may vary depending upon the intended application (in vitro or in vivo), the subject and disease condition being treated, e.g., the weight and age of the subject, the severity of the disease condition, the manner of administration and the like. In some embodiments, a therapeutically effective amount refers to a dose that will induce a particular response in target cells. The specific dose will vary depending on the particular compounds chosen, the dosing regimen to be followed, whether it is administered in combination with other compounds, timing of administration, the tissue to which it is administered, and the physical delivery system in which it is carried.

In treatment methods of the disclosure, a therapeutically effective amount of a compound of the disclosure (or a pharmaceutically acceptable salt thereof) is administered to a subject suffering from or diagnosed as having such a disease or disorder. Therapeutically effective amounts or doses of the compounds of the present disclosure may be ascertained by routine methods such as modeling, dose escalation studies or clinical trials, and by taking into consideration factors such as, e.g., the mode or route of administration or drug delivery, the pharmacokinetics of the compound, the severity and course of the disease or disorder, the subject's previous or ongoing therapy, the subject's health status and response to drugs, and the judgment of the treating physician. An example of a dose is in the range of from about 0.001 to about 100 mg of compound per kg of subject's body weight per day, in single or divided dosage units (e.g., BID, TID, QID). For a 70-kg human, an illustrative range for a suitable dosage amount is from about 0.05 to about 7 g/day.

In addition, the compounds of the disclosure may be used in combination with additional active ingredients in the treatment of the diseases or disorders disclosed herein. The additional active ingredients may be coadministered separately with a compound of the disclosure or included with such an agent in a pharmaceutical composition according to the disclosure. The combination may serve to increase efficacy (e.g., by including in the combination a compound potentiating the potency or effectiveness of an active agent according to the disclosure), decrease one or more side effects, or decrease the required dose of the active agent according to the disclosure.

In some aspects, the disclosure is directed to a method of treating a BCAA-catabolism-related disease comprising administering a compound of the disclosure.

In some embodiments, the BCAA-catabolism-related disease is methylmalonic acidemia (MMA), propionic acidemia (PA), isovaleric acidemia (IVA), maple syrup urine disease, methylmalonic semialdehyde (MMSDH) deficiency, 3-hydroxyisobutyrate dehydrogenase (HIBADH) deficiency, 3-hydroxyisobutyryl-CoA hydrolase (HIBCH) deficiency, isobutyryl-CoA (IBD) deficiency, acetyl-CoA carboxylase 1 (ACC) deficiency, mitochondrial short-chain enoyl-CoA hydratase 1 (ECHS1) deficiency, methylbutyryl CoA dehydrogenase (SBCAD) deficiency (also known as short/branched chain acyl-CoA dehydrogenase deficiency), beta-ketothiolase deficiency (BKT), hydroxy-3-methylglutaconyl-CoA lysase (HMGCL) deficiency, 3-methylglutaconyl-CoA-hydratase deficiency (MGA), 3-methylcrotonyl-CoA (3-MCC) deficiency, glutaric aciduria type 1 (GA1), 3-hydroxy-3-methylglutaryl-CoA lyase deficiency (MGA), 3-methylglutaconic acidemia, D-2 hydroxyglutaric aciduria (D2-HGA), isobutyryl-CoA dehydrogenase deficiency, 3-hydroxyisobutyric aciduria (ICBD), L-2-hydroxy-glutaricaciduria (L2HGA), malonyl-CoA decarboxylase deficiency aka malonic acidemia (MA), multiple carboxylase deficiency (MCD, holocarboxylase synthetase), or 2-methyl-3-hydroxybutyryl-CoA dehydrogenase (MHBD) deficiency.

In some embodiments, the BCAA-catabolism-related disease is an organic acidemia.

In some embodiments, the BCAA-catabolism-related disease is methylmalonic acidemia (MMA), propionic acidemia (PA), isovaleric acidemia (IVA), or maple syrup urine disease.

In some embodiments, the BCAA-catabolism-related disease is methylmalonic acidemia (MMA).

In some embodiments, the BCAA-catabolism-related disease is propionic acidemia (PA).

In some embodiments, the BCAA-catabolism-related disease is isovaleric acidemia (IVA).

In some embodiments, the BCAA-catabolism-related disease is maple syrup urine disease.

In some embodiments, the methods of the disclosure reduce and/or ameliorate the symptoms of the BCAA-catabolism-related disease in the subject.

In some aspects, the disclosure provides methods of reducing toxic BCAA metabolites in one or more bodily fluids of the subject such as the blood or urine of a subject, comprising administering a therapeutically effective amount of a BCAT2 inhibitor of the disclosure. As used here, the term “reducing” means that the BCAA metabolite levels in the subject's bodily fluids are lower after being administered a therapeutically effective amount of a BCAT2 inhibitor of the disclosure than the level prior to administration. Methods of measuring the levels of BCAA metabolites in blood and urine and normal ranges of those metabolites in such bodily fluids are known in the art.

In some embodiments, the methods cause a decrease in toxic branched chain amino acid (BCAA) metabolite levels.

In some embodiments, the toxic metabolite is ammonia, propionic acid, 2-methylcitric acid, 3-hydroxypropionic acid, propionyl-CoA, methylmalonic acid, methylmalonyl-CoA or isovaleric acid. In other embodiments, the toxic metabolite is propionic acid, 2-methylcitric acid, 3-hydroxypropionic acid, propionyl-CoA, methylmalonic acid, methylmalonyl-CoA or isovaleric acid.

In some embodiments, the toxic metabolites are allosteric enzyme inhibitors.

In some embodiments, the methods of the disclosure cause an increase in BCAA levels in the blood of a subject. As used here, the term “increase” means that the BCAA level in the subject's blood is greater after being administered a therapeutically effective amount of a BCAT2 inhibitor of the disclosure than the level prior to the administration. Methods of measuring the blood levels of BCAAs are known in the art.

In some embodiments, the BCAA is one or more of leucine, isoleucine, or valine.

In some embodiments, the methods of the disclosure modulate metabolic flux through a BCAA-catabolism pathway. As used herein, “metabolic flux” refers to the rate of passage of molecules through a metabolic pathway. The term modulate means to change, i.e., to increase or decrease. Methods of measuring metabolic flux are known in the art. Thus, in such embodiments, the metabolic flux through a BCAA-catabolism pathway is different before and after administering a BCAT2 inhibitor of the disclosure.

In some embodiments, the methods of the disclosure decrease metabolic flux through a BCAA-catabolism pathway.

In some embodiments, the methods of the disclosure decrease BCAT2-mediated metabolic flux through a BCAA-catabolism pathway.

In some embodiments, the reduction of BCAT2-mediated flux occurs by reduction or inhibition of leucine catabolism. In other embodiments, leucine catabolism is reduced or inhibited at one or more enzymes in the BCAA-catabolism pathway, wherein the enzyme is branched chain α-ketoacid dehydrogenase complex (BCKDH complex), isovaleryl CoA dehydrogenase (IVD), 3-methylcrotonyl-CoA carboxylase (3MCC), 3-methylglutaconyl-CoA hydratase (3MGA), 3-hydroxy-3-methylglutaryl-CoA lyase (HMGL), acetyl-CoA carboxylase (AC), or malonyl-CoA decarboxylase (MA).

In some embodiments, the reduction of BCAT2-mediated flux occurs by reduction or inhibition of isoleucine catabolism. In some embodiments, isoleucine catabolism is reduced or inhibited at one or more enzymes in the BCAA-catabolism pathway, wherein the enzyme is BCKDH complex, methylbutyryl CoA dehydrogenase (SBCAD), mitochondrial short-chain enoyl-CoA hydratase, 2-methyl-3-hydroxyisobutyryl-CoA dehydrogenase (MHBD), acetoacetyl-CoA thiolase/beta-ketothiolase (T2/ACAT1), AC, MA, propionyl-CoA carboxylase (PCC), or methylmalonyl-CoA mutase (MUT), or succinyl-CoA ligase (SUCLA).

In some embodiments, the reduction of BCAT2-mediated flux occurs by reduction or inhibition of valine catabolism. In some embodiments, valine catabolism is reduced or inhibited at one or more enzymes in the BCAA-catabolism pathway, wherein the enzyme is BCKDH complex, isobutyryl-CoA dehydrogenase (IBDH), mitochondrial short-chain enoyl-CoA hydratase, 3-hydroxyisobutyryl-CoA deacylase (hydrolase) (HIBDA), 3-hydroxyisobutyrate dehydrogenase (HIBDH), methylmalonic semialdehyde dehydrogenase (MMSDH), PCC, MUT, or SUCLA.

In some aspects, the disclosure provides methods of reducing a toxic load burden in cells of a subject having a BCAA-catabolism-related disease, comprising administering a therapeutically effective amount of a BCAT2 inhibitor of the disclosure.

The present disclosure provides compounds of the disclosure as BCAT2 inhibitors for the treatment of diseases and conditions wherein inhibition of BCAT2 has a beneficial effect. Compounds of the disclosure typically inhibit BCAT2 (IC₅₀) in a concentration less than 20 μM, e.g., less than 10 μM, less than 5 μM, less than 3 μM, and less than 2 μM, less than about 1 μM, less than about 0.5 μM, less than about 0.1 μM, less than about 0.05 μM, or less than about 0.01 μM. In one embodiment, the present disclosure relates to a method of treating a subject suffering from a disease or condition wherein inhibition of BCAT2 provides a benefit comprising administering a therapeutically effective amount of a compound of the disclosure to a subject in need thereof.

In some embodiments, the compounds disclosed herein selectively inhibit BCAT2 as compared to branched-chain amino acid aminotransferase 1 (BCAT1). BCAT1 is the other isoenzyme of BCAA aminotransferase and is known as the cytosolic form, whereas BCAT2 is known as the mitochondrial form. The selectivity of a given compound for BCAT2 vs. BCAT1 can be measured by comparing the relative inhibitory activity (e.g., IC₅₀) of the compound in BCAT2 to that in BCAT1. A compound is selective for BCAT2 over BCAT1 when the compound has a greater potency of inhibiting BCAT2 than of inhibiting BCAT1. Methods of determining relative selectivity of inhibitors are known in the art and are described herein. In some embodiments, the inhibitors of the disclosure are selective for BCAT2 over BCAT1. In some embodiments, the inhibitors of the disclosure are about 2-100 times more selective for BCAT2 over BCAT1, such as, for example, about 2, 3, 4, 5, 6, 7, 8, 9, 10, 15, 20, 25, 30, 35, 40, 45, 50, 55, 60, 65, 70, 75, 80, 85, 90, 95, or 100 times more selective for BCAT2 over BCAT1.

In some aspects, the disclosure is directed to methods of reducing plasma methylmalonic acid in a subject having an elevated methylmalonic acid level comprising administering to the subject a therapeutically effective amount of a compound of the disclosure. As used here, “reducing” means that the plasma methylmalonic acid level in the subject is lower after administering the compound of the disclosure than before administering the compound of the disclosure. Methods of measuring plasma methylmalonic acid levels in plasma are known in the art. See, e.g., Manoli I, et al. Isolated Methylmalonic Acidemia. 2005 Aug. 16 [Updated 2016 Dec. 1]. In: Adam M P, Ardinger H H, Pagon R A, et al., editors. GeneReviews® [Internet]. Seattle (WA): University of Washington, Seattle; 1993-2021. As used herein, “elevated methylmalonic acid level” refers to a plasma methylmalonic acid level that is higher than the normal level expected for a given subject. See Anna-Kaisa Niemi, et al., Treatment of Methylmalonic Acidemia by Liver or Combined Liver-Kidney Transplantation, The Journal of Pediatrics, Volume 166, Issue 6, 2015, 1455-1461.e1. Normal blood level of methylmalonic acid in humans is <0.27 μmol/L. Elevated blood levels of methylmalonic acid levels in humans range from 5-1000 μmol/L, depending in part on the enzyme deficiency that is causing the patient's MMA.

In some aspects, the disclosure is directed to methods of reducing blood ammonia in a subject having an elevated ammonia level (hyperammonemia) comprising administering to the subject a therapeutically effective amount of a compound of the disclosure. As used here, “reducing” means that the blood ammonia level in the subject is lower after administering the compound of the disclosure than before administering the compound of the disclosure. Normal blood level of ammonia in humans generally range from 11-32 μmol/L. A blood ammonia level above 75 μmol/L is associated with symptomatic decompensation and a blood ammonia level above 200 μmol/L is associated with impaired vigilance. See Nicola Longo et al., Hyperammonemia in classic organic acid: a review of the literature and two case histories, Orphanet Journal of Rare Diseases, Volume 13, Issue 1, 2018, 219.

In some aspects, the disclosure is directed to methods of reducing blood 2-methylcitrate in a subject having an elevated 2-methylcitrate level comprising administering to the subject a therapeutically effective amount of a compound of the disclosure. As used here, “reducing” means that the blood 2-methylcitrate level in the subject is lower after administering the compound of the disclosure than before administering the compound of the disclosure. Normal blood level of 2-methylcitrate in humans is ≤0.63 μmol/L, whereas blood 2-methylcitrate in MMA and PA patients range between 1.0-12.0 μmol/L according to one report. Osama Y. Al-Dirbashi et al., Assessment of methylcitrate and methylcitrate to citrate ratio in dried blood spots as biomarkers for inborn errors of propionate metabolism, Volume 9, 2019, 123666.

In some aspects, the methods of the disclosure are performed in combination with other treatment strategies.

In some embodiments, the other treatment strategies are aimed at reducing the levels of toxic metabolites.

In some embodiments, the treatment strategies aimed at reducing the levels of toxic metabolites are directed to reducing the supply of BCAAs. Such strategies include isoleucine, valine, threonine and methionine restriction as part of a protein-restricted diet (with or without amino acid supplementation), eating a high caloric diet during illness, administering antibiotics, administering laxatives, or reducing odd-chain fatty acids and cholesterol.

In other embodiments, the treatment strategies aimed at reducing the levels of toxic metabolites are directed to increasing enzyme activity. Such embodiments include administering hydroxycobalamin, solid organ transplantation (liver or combined liver-kidney transplantation), gene therapy, promotion of premature stop codon read-through, messenger RNA therapy, pharmacological stabilization of enzyme activity, enzyme replacement therapy, or hepatocyte transplantation.

In other embodiments, the treatment strategies aimed at reducing the levels of toxic metabolites are directed to increasing disposal of toxic metabolites. Such embodiments include administering a carbamoyl phosphate synthetase 1 (CPS 1) activator, administering N-carbamylglutamate (CARBAGLU®), administering sodium benzoate, extracorporeal detoxification, administering L-carnitine, or administering sodium bicarbonate.

In some embodiments, the other treatment strategies are aimed at preventing or treating mitochondrial energetic failure and increase of reactive oxygen species (ROS) formation.

In some embodiments, other treatment strategies aimed at preventing or treating mitochondrial energetic failure and increase of reactive oxygen species (ROS) formation are directed to effecting energetic supply. Such embodiments include administering succinate, citric acid, or creatine.

In some embodiments, other treatment strategies aimed at preventing or treating mitochondrial energetic failure and increase of reactive oxygen species (ROS) formation are directed to cofactor supplementation. Such embodiments include administering pyridoxine (vitamin B6) or administering thiamine (vitamin B1). In some embodiments, other treatment strategies aimed at preventing or treating mitochondrial energetic failure and increase of reactive oxygen species (ROS) formation are directed to anti-oxidant supplementation. Such embodiments include administering coenzyme Q10, ascorbic acid (vitamin C), alpha-tocopherol (vitamin E), CB agonist WIN, S-allylcysteine, gluthathione, GM1 ganglioside, melatonin, mitoQ, resveratrol, tiron, or trolox.

In some embodiments, the other treatment strategies that are aimed at preventing or treating mitochondrial energetic failure and increase of reactive oxygen species (ROS) formation are directed to electron transfer mediator supplementation. Such embodiments include administering methylene blue.

In some embodiments, the other treatment strategies that are aimed at preventing or treating mitochondrial energetic failure and increase of reactive oxygen species (ROS) formation are directed to administering NMDA receptor antagonists. Such embodiments include administering celecoxib, fish oil, kynurenic acid, or MK-801.

In some embodiments, the other treatment strategies include administering an inhibitor of an ammo acid transporter, optionally with a SLC6A19 inhibitor. In further embodiments, the amino acid transporter is B⁰AT1 (also referred to as SLC6A19), and the inhibitor is a SLC6A19 inhibitor. Examples of SLC6A19 inhibitors include nimesulide, benztropine, NSC63912, NSC22789, cinromide, CB3, E62, and the like.

In some embodiments, performing the methods of the disclosure in combination with the other treatment strategies results in levels of branched chain amino acids that are reduced compared to treatment without performance of the other treatment strategy.

In some embodiments, the disclosure is directed to the following aspects:
Aspect 1. A compound, wherein said compound is a compound of Formula IA or a compound of Formula IB:

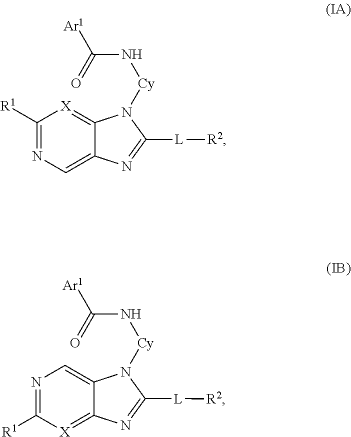

or a pharmaceutically acceptable salt thereof, wherein
R¹ is optionally substituted 5- or 6-membered heteroaryl, optionally substituted imidazolidinonyl, optionally substituted pyrrolidinonyl, optionally substituted dihydropyrazolonyl, optionally substituted dihydropyrrolonyl, optionally substituted dihydroimidazolonyl, or optionally substituted dihydrotriazolonyl;
X is CH or N;
Cy is optionally substituted C₅-C₇ cycloalkyl or optionally substituted 6- or 7-membered heterocycloalkyl;
L is a bond, —O—, —NR^{a}—, or optionally substituted C₁-C₄alkylene;
   R^{a} is H or C₁-C₄alkyl;
R² is optionally substituted phenyl, optionally substituted pyridinyl, optionally substituted 5-membered heteroaryl, optionally substituted heterocycloalkyl, optionally substituted C₁-C₆alkyl, optionally substituted C₁-C₆alkoxy, optionally substituted C₁-C₆hydroxyalkyl, optionally substituted C₁-C₆haloalkyl, or optionally substituted C₁-C₆haloalkoxy; and
Ar¹ is an optionally substituted five-membered heteroaryl.
Aspect 2. The compound according to aspect 1, or a pharmaceutically acceptable salt thereof, wherein the compound is a compound of Formula IA.
Aspect 3. The compound according to aspect 1, or a pharmaceutically acceptable salt thereof, wherein the compound is a compound of Formula IB.
Aspect 4. The compound according to aspect 2 or aspect 3, or a pharmaceutically acceptable salt thereof, wherein X is CH.
Aspect 5. The compound according to aspect 2 or aspect 3, or a pharmaceutically acceptable salt thereof, wherein X is N.
Aspect 6. The compound according to any one of aspects 1-5, or a pharmaceutically acceptable salt thereof, wherein Cy is optionally substituted C₅-C₇ cycloalkyl.
Aspect 7. The compound according to aspect 6, or a pharmaceutically acceptable salt thereof, wherein Cy is unsubstituted C₅-C₇ cycloalkyl.
Aspect 8. The compound according to aspect 6, or a pharmaceutically acceptable salt thereof, wherein Cy is C₅-C₇ cycloalkyl substituted with one or more of OH, halo, cyano, —C₁-C₃alkyl, C₁-C₃haloalkyl, C₁-C₃alkoxy, C₁-C₃haloalkoxy, —NH₂, —NH(C₁-C₆alkyl), or —N(C₁-C₆alkyl)₂.
Aspect 9. The compound according to aspect 8, or a pharmaceutically acceptable salt thereof, wherein Cy is C₅-C₇ cycloalkyl substituted with OH.
Aspect 10. The compound according to aspect 6, or a pharmaceutically acceptable salt thereof, wherein the optionally substituted C₅-C₇ cycloalkyl is an optionally substituted cyclohexyl or an optionally substituted cycloheptyl.
Aspect 11. The compound according to aspect 6, or a pharmaceutically acceptable salt thereof, wherein the optionally substituted C₅-C₇ cycloalkyl is an optionally substituted cyclopentyl.
Aspect 12. The compound according to aspect 11, or a pharmaceutically acceptable salt thereof, wherein the optionally substituted cyclopentyl is an unsubstituted cyclopentyl.
Aspect 13. The compound according to aspect 6, or a pharmaceutically acceptable salt thereof, wherein the optionally substituted C₅-C₇ cycloalkyl is an optionally substituted cyclohexyl.
Aspect 14. The compound according to aspect 13, or a pharmaceutically acceptable salt thereof, wherein the cyclohexyl is substituted with one or more OH, halo, cyano, C₁-C₃alkyl, C₁-C₃haloalkyl, C₁-C₃alkoxy, C₁-C₃haloalkoxy, NH₂, NH(C₁-C₆alkyl), or N(C₁-C₆alkyl)₂.
Aspect 15. The compound according to aspect 14, or a pharmaceutically acceptable salt thereof, wherein the cyclohexyl is substituted with OH.
Aspect 16. The compound according to aspect 13, or a pharmaceutically acceptable salt thereof, wherein the optionally substituted cyclohexyl is an unsubstituted cyclohexyl.
Aspect 17. The compound according to aspect 6, or a pharmaceutically acceptable salt thereof, wherein the optionally substituted C₅-C₇ cycloalkyl is an optionally substituted cycloheptyl.
Aspect 18. The compound according to aspect 17, or a pharmaceutically acceptable salt thereof, wherein the optionally substituted cycloheptyl is an unsubstituted cycloheptyl.
Aspect 19. The compound according to any one of aspects 1-5, or a pharmaceutically acceptable salt thereof, wherein Cy is an optionally substituted 6-membered heterocycloalkyl.
Aspect 20. The compound according to aspect 19, or a pharmaceutically acceptable salt thereof, wherein Cy is an optionally substituted piperidinyl, an optionally substituted tetrahydropyranyl, an optionally substituted thianyl, an optionally substituted thianyl sulfone, an optionally substituted thianyl sulfoxide, or an optionally substituted 1-oxo-1-imino-1-thiacyclohexyl.
Aspect 21. The compound according to any one of aspects 19-20, or a pharmaceutically acceptable salt thereof, wherein Cy is an optionally substituted piperidinyl.
Aspect 22. The compound according to any one of aspects 19-20, or a pharmaceutically acceptable salt thereof, wherein Cy is an optionally substituted tetrahydropyranyl.
Aspect 23. The compound according to any one of aspects 1-5, or a pharmaceutically acceptable salt thereof, wherein Cy is an optionally substituted 7-membered heterocycloalkyl.
Aspect 24. The compound according to aspect 23, or a pharmaceutically acceptable salt thereof, wherein the optionally substituted 7-membered heterocycloalkyl is an optionally substituted oxazepanyl, an optionally substituted thiepanyl, an optionally substituted thiepanyl-1-oxide, an optionally substituted thiepanyl-1,1-dioxide, an optionally substituted azepanyl, or an optionally substituted 1-imino-1-oxide-thiepanyl.
Aspect 25. The compound according to any one of the preceding aspects, or a pharmaceutically acceptable salt thereof, wherein R¹ is optionally substituted 5-membered heteroaryl.
Aspect 26. The compound according to aspect 25, or a pharmaceutically acceptable salt thereof, wherein the 5-membered heteroaryl is an oxadiazolyl, oxazolyl, pyrazolyl, triazolyl, or tetrazolyl.
Aspect 27. The compound according to any one of aspects 1-24, or a pharmaceutically acceptable salt thereof, wherein R¹ is optionally substituted 6-membered heteroaryl.
Aspect 28. The compound according to aspect 27, or a pharmaceutically acceptable salt thereof, wherein the optionally substituted 6-membered heteroaryl is optionally substituted pyridinyl.
Aspect 29. The compound according to any one of aspects 1-24, or a pharmaceutically acceptable salt thereof, wherein R¹ is optionally substituted imidazolidinonyl.
Aspect 30. The compound according to any one of aspects 1-24, or a pharmaceutically acceptable salt thereof, wherein R¹ is optionally substituted pyrrolidinonyl.
Aspect 31. The compound according to any one of aspects 1-30, or a pharmaceutically acceptable salt thereof, wherein L is a bond.
Aspect 32. The compound according to any one of aspects 1-30, or a pharmaceutically acceptable salt thereof, wherein L is —O—.
Aspect 33. The compound according to any one of aspects 1-30, or a pharmaceutically acceptable salt thereof, wherein L is —NR^{a}—, wherein R^{a} is H or C₁-C₄alkyl.
Aspect 34. The compound according to any one of aspects 1-30, or a pharmaceutically acceptable salt thereof, wherein L is optionally substituted C₁-C₄alkylene.
Aspect 35. The compound according to any one of aspects 1-34, or a pharmaceutically acceptable salt thereof, wherein R² is optionally substituted phenyl.
Aspect 36. The compound according to any one of aspects 1-34, or a pharmaceutically acceptable salt thereof, wherein R² is optionally substituted pyridinyl.
Aspect 37. The compound according to any one of aspects 1-34, or a pharmaceutically acceptable salt thereof, wherein R² is optionally substituted 5-membered heteroaryl.
Aspect 38. The compound according to aspect 37, or a pharmaceutically acceptable salt thereof, wherein the 5-membered heteroaryl is triazolyl, pyrazolyl, oxazolyl, or isoxazolyl.
Aspect 39. The compound according to any one of aspects 1-34, or a pharmaceutically acceptable salt thereof, wherein R² is optionally substituted heterocycloalkyl.
Aspect 40. The compound according to aspect 39, or a pharmaceutically acceptable salt thereof, wherein the heterocycloalkyl is tetrahydropyranyl or piperidinyl.
Aspect 41. The compound according to any one of aspects 1-34, or a pharmaceutically acceptable salt thereof, wherein R² is optionally substituted C₁-C₆alkyl.
Aspect 42. The compound according to aspect 41, or a pharmaceutically acceptable salt thereof, wherein the C₁-C₆alkyl is CH₃, CH(CH₃)₂, or CH₂CH(CH₃)₂.
Aspect 43. The compound according to any one of aspects 1-34, or a pharmaceutically acceptable salt thereof, wherein R² is optionally substituted C₁-C₆alkoxy.
Aspect 44. The compound according to aspect 43, or a pharmaceutically acceptable salt thereof, wherein the C₁-C₆alkoxy is OCH₃, or OCH(CH₃)₂.
Aspect 45. The compound according to any one of aspects 1-34, or a pharmaceutically acceptable salt thereof, wherein R² is optionally substituted C₁-C₆hydroxyalkyl.
Aspect 46. The compound according to aspect 45, or a pharmaceutically acceptable salt thereof, wherein the C₁-C₆hydroxyalkyl is C(OH)(CH₃)₂.
Aspect 47. The compound according to any one of aspects 1-34, or a pharmaceutically acceptable salt thereof, wherein R² is optionally substituted C₁-C₆haloalkyl.
Aspect 48. The compound according to aspect 47, or a pharmaceutically acceptable salt thereof, wherein the C₁-C₆haloalkyl is CH(CH₃)(CF₃).
Aspect 49. The compound according to any one of aspects 1-34, or a pharmaceutically acceptable salt thereof, wherein R² is optionally substituted C₁-C₆haloalkoxy.
Aspect 50. The compound according to aspect 49, or a pharmaceutically acceptable salt thereof, wherein the C₁-C₆haloalkoxy is OCH₂CF₃, or OCH(CH₃)(CF₃).
Aspect 51. The compound according to any one of the preceding aspects, or a pharmaceutically acceptable salt thereof, wherein Ar¹ is an optionally substituted thiophenyl or an optionally substituted thiazolyl.
Aspect 52. The compound according to any one of aspects 1-2, 4-10, 13-16, or 25-51, wherein the compound of Formula IA is a compound of Formula IA-1:

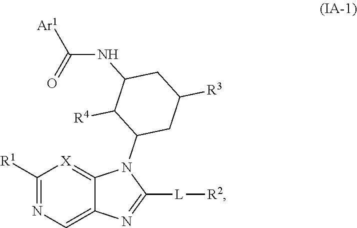

or a pharmaceutically acceptable salt thereof, wherein R³ is H or OH; R⁴ is H or OH, and wherein at least one of R³ and R⁴ is H.
Aspect 53. The compound according to aspect 52, wherein the compound of Formula IA-1 is a compound of Formula IA-1a:

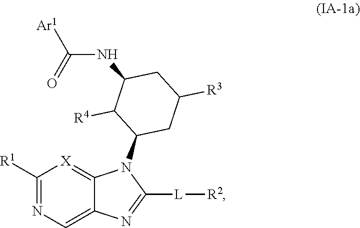

or a pharmaceutically acceptable salt thereof.
Aspect 54. The compound according to any one of aspects 1-2, 4-10, 17, 18, or 25-51, wherein the compound of Formula IA is a compound of Formula IA-2 or Formula IA-3:

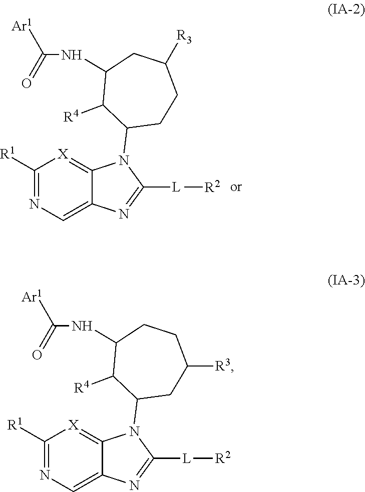

or a pharmaceutically acceptable salt thereof, wherein R³ is H or OH; R⁴ is H or OH, and wherein at least one of R³ and R⁴ is H.
Aspect 55. The compound according to aspect 54, wherein the compound of Formula IA-2 is a compound of Formula IA-2a, or a pharmaceutically acceptable salt thereof, and the compound of Formula IA-3 is a compound of Formula IA-3a, or a pharmaceutically acceptable salt thereof;

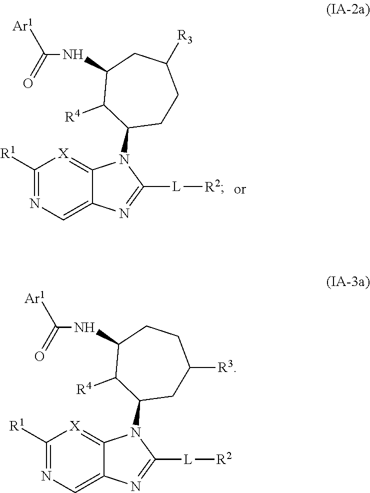

Aspect 56. The compound according to any one of aspects 1-2, 4-5, 19-22, and 25-51, wherein the compound of Formula IA is a compound of Formula IA-4:

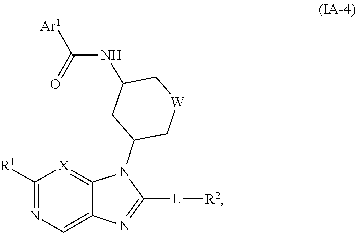

or a pharmaceutically acceptable salt thereof, wherein W is N—R⁵, S, SO, SO₂, or O; and R⁵ is H, C₁-C₆alkyl, C(O)C₁-C₆alkyl, ═O, OH, or C₁-C₆alkoxy.
Aspect 57. The compound according to aspect 56, wherein the compound of Formula IA is a compound of Formula IA-4a:

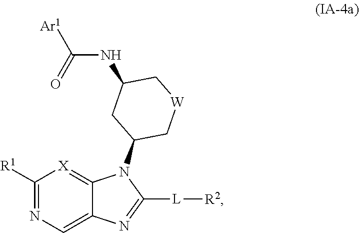

or a pharmaceutically acceptable salt thereof.
Aspect 58. The compound according to aspect 56 or aspect 57, or a pharmaceutically acceptable salt thereof, wherein W is N—R⁵.
Aspect 59. The compound according to aspect 56 or aspect 57, or a pharmaceutically acceptable salt thereof, wherein W is 0.
Aspect 60. The compound according to any one of aspects 1-2, and 4-51, wherein the compound of Formula IA is a compound of Formula IA-5:

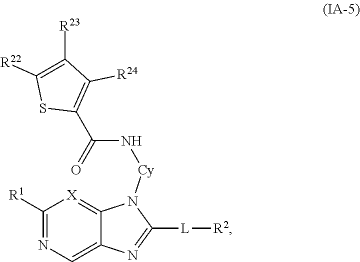

or a pharmaceutically acceptable salt thereof, wherein R²² is halo or optionally substituted C₂-C₆alkynyl; and R²³ and R²⁴ are hydrogen.
Aspect 61. The compound according to any one of aspects 1-2, and 4-51, wherein the compound of Formula IA is a compound of Formula IA-6:

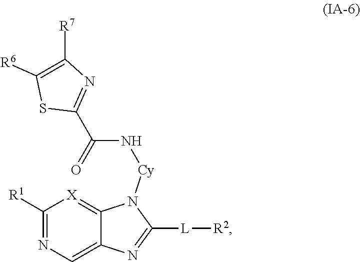

or a pharmaceutically acceptable salt thereof, wherein R⁶ is halo, C₁-C₆haloalkyl, cyano, or optionally substituted C₂-C₆alkynyl; and R⁷ is hydrogen.
Aspect 62. The compound according to any one of aspects 1-2, 4-31, 35, and 51, wherein the compound of Formula IA is a compound of Formula IA-7:

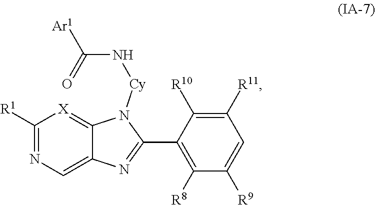

or a pharmaceutically acceptable salt thereof, wherein R⁸, R⁹, R¹⁰, and R¹¹ are each independently H, halo, C₁-C₄alkyl, C₃-C₄cycloalkyl, C₁-C₄alkoxy, CN, NR^{b}R^{c}, C(O)NR^{b}R^{c}, C(O)OC₁-C₄alkyl, COOH, or C₁₋₄alkylenecarboxylic acid; and
   each R^{b} and each R^{c} is independently H or C₁-C₄alkyl.
Aspect 63. The compound according to aspect 62, or a pharmaceutically acceptable salt thereof, wherein at least two of R⁸, R⁹, R¹⁰, and R¹¹ are H.
Aspect 64. The compound according to aspect 62, or a pharmaceutically acceptable salt thereof, wherein at least three of R⁸, R⁹, R¹⁰, and R¹¹ are H.
Aspect 65. The compound according to any one of aspects 62-64, or a pharmaceutically acceptable salt thereof, wherein at least one of R⁸, R⁹, R¹⁰, and R¹¹ is F, CH₃, OCH₃, NH₂, or CN.
Aspect 66. The compound according to any one of aspects 1-2, 4-31, 36, and 51, wherein the compound of Formula IA is a compound of Formula IA-8:

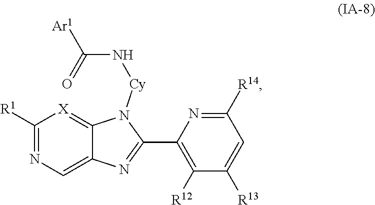

or a pharmaceutically acceptable salt thereof, wherein R¹², R¹³, and R¹⁴ are each independently H, halo, C₁-C₄alkyl, C₃-C₄cycloalkyl, C₁-C₄alkoxy, CN, NR^{b}R^{c}, C(O)NR^{b}R^{c}, C(O)OC₁-C₄alkyl, COOH, or C₁₋₄alkylenecarboxylic acid; and
   each R^{b} and each R^{c} is independently H or C₁-C₄alkyl.
Aspect 67. The compound according to aspect 66, or a pharmaceutically acceptable salt thereof, wherein at least two of R¹², R¹³, and R¹⁴ are H.
Aspect 68. The compound according to aspect 66 or aspect 67, or a pharmaceutically acceptable salt thereof, wherein at least one of R¹², R¹³, and R¹⁴ is F, CH₃, OCH₃, NH₂, or CN.
Aspect 69. The compound according to any one of aspects 1-2, 4-26, and 31-51, wherein the compound of Formula IA is a compound of Formula IA-9:

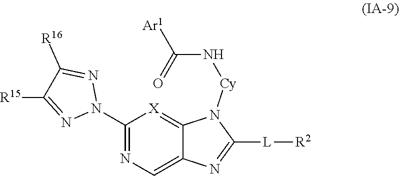

or a pharmaceutically acceptable salt thereof, wherein R¹⁵ and R¹⁶ are each independently H, halo, C₁-C₆alkyl, C(O)OC₁-C₆alkyl, or CONR^{b}R^{c}; and
each R^{b} and each R^{c} is independently H or C₁-C₄alkyl.
Aspect 70. The compound according to aspect 69, or a pharmaceutically acceptable salt thereof, wherein at least one of R¹⁵ and R¹⁶ is H.
Aspect 71. The compound according to aspect 69, or a pharmaceutically acceptable salt thereof, wherein one of R¹⁵ and R¹⁶ is halo or CH₃.
Aspect 72. The compound according to any one of aspects 1-2, 4-26, and 31-51, wherein the compound of Formula IA is a compound of Formula IA-10:

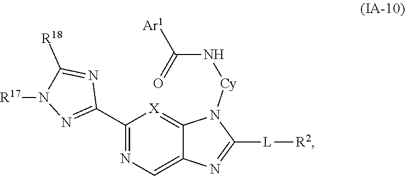

or a pharmaceutically acceptable salt thereof, wherein R¹⁷ is H or C₁-C₆alkyl;
R¹⁸ is H, halo, C₁-C₆alkyl, C(O)OC₁-C₆alkyl, or CONR^{b}R^{c}; and each R^{b} and each R^{c} is independently H or C₁-C₄alkyl.
Aspect 73. The compound according to any one of aspects 1-2, 4-26, and 31-51, wherein the compound of Formula IA is a compound of Formula IA-11:

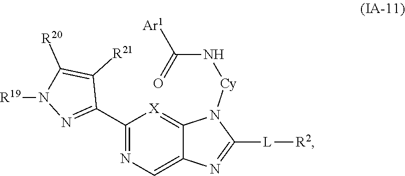

or a pharmaceutically acceptable salt thereof, wherein R is H or C₁-C₆alkyl;
and R²⁰ and R²¹ are each independently H, halo, C₁-C₆alkyl, C(O)OC₁-C₆alkyl, or CONR^{b}R^{c}; and each R^{b} and each R^{c} is independently H or C₁-C₄alkyl.
Aspect 74. The compound according to any one of aspects 1, 3-10, 13-16 or 25-51, wherein the compound of Formula IB is a compound of Formula IB-1:

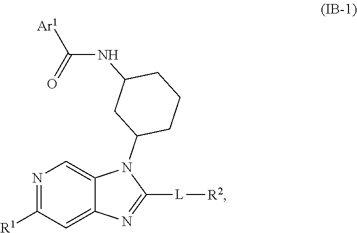

or a pharmaceutically acceptable salt thereof.
Aspect 75. The compound according to aspect 74, wherein the compound of Formula IB-1 is a compound of Formula IB-1a:

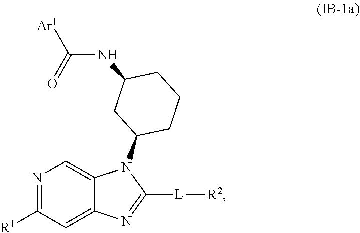

or a pharmaceutically acceptable salt thereof.
Aspect 76. The compound according to aspect 74 or aspect 75, or a pharmaceutically acceptable salt thereof, wherein R¹ is optionally substituted triazolyl, optionally substituted oxadiazolyl, or optionally substituted pyrazolyl.
Aspect 77. The compound according to any one of aspects 74-76, or a pharmaceutically acceptable salt thereof, wherein L is a bond and R² is optionally substituted phenyl or optionally substituted pyridinyl.
Aspect 78. The compound according to any one of aspects 74-77, or a pharmaceutically acceptable salt thereof, wherein Ar¹ is optionally substituted thiophenyl or optionally substituted thiazolyl.
Aspect 79. A pharmaceutical composition comprising a compound according to any one of aspects 1-78, or a pharmaceutically acceptable salt thereof, and a pharmaceutically acceptable excipient.
Aspect 80. A method of reducing plasma methylmalonic acid in a subject having an elevated methylmalonic acid level comprising administering to the human a therapeutically effective amount of a compound of any one of aspects 1-78.
Aspect 81. A method of treating a BCAA-catabolism-related diseases in a subject in need thereof comprising administering to the subject a therapeutically effective amount of a compound, or a pharmaceutically acceptable salt thereof, of any one of aspects 1-78.
Aspect 82. The method according to aspect 81, wherein the BCAA-catabolism-related disease is methylmalonic acidemia (MMA), propionic acidemia (PA), isovaleric acidemia (IVA), maple syrup urine disease, methylmalonic semialdehyde (MMSDH) deficiency, 3-hydroxyisobutyrate dehydrogenase (HIBADH) deficiency, 3-hydroxyisobutyryl-CoA hydrolase (HIBCH) deficiency, isobutyryl-CoA (IBD) deficiency, acetyl-CoA carboxylase 1 (ACC) deficiency, mitochondrial short-chain enoyl-CoA hydratase 1 (ECHS1) deficiency, methylbutyryl CoA dehydrogenase (SBCAD) deficiency, beta-ketothiolase deficiency (BKT), hydroxy-3-methylglutaconyl-CoA lysase (HMGCL) deficiency, 3-methylglutaconyl-CoA-hydratase deficiency, or 3-methylcrotonyl-CoA (3-MCC) deficiency.

In other embodiments, the disclosure is directed to the following aspects:
Aspect 83. A compound, wherein said compound is a compound of Formula IA or a compound of Formula IB:

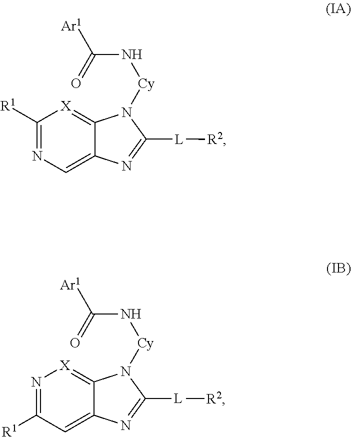

or a pharmaceutically acceptable salt thereof, wherein:
R¹ is optionally substituted heteroaryl, optionally substituted aryl, or optionally substituted 4-, 5- or 6-membered heterocycloalkyl;
X is CH or N;
Cy is optionally substituted C₅-C₇ cycloalkyl or optionally substituted 6- or 7-membered heterocycloalkyl;
L is a bond, —O—, —NR^{a}—, or optionally substituted C₁-C₄alkylene; R^{a} is hydrogen or C₁-C₄alkyl;
R² is optionally substituted phenyl, optionally substituted pyridinyl, optionally substituted 5-membered heteroaryl, optionally substituted heterocycloalkyl, optionally substituted C₁-C₆alkyl, optionally substituted C₁-C₆alkoxy, optionally substituted C₁-C₆hydroxyalkyl, optionally substituted C₁-C₆haloalkyl, optionally substituted C₁-C₆haloalkoxy, optionally substituted C₃-C₆cycloalkyl, or NR^{f}R^{g};
R^{f} and R^{g} are each independently hydrogen, C₁-C₆alkyl, C₁-C₆haloalkyl, OH, C₁-C₄alkoxy, C₁-C₆hydroxyalkyl, or C₃-C₆cycloalkyl; and
Ar¹ is an optionally substituted five-membered heteroaryl.
Aspect 84. The compound according to aspect 83, or a pharmaceutically acceptable salt thereof, wherein the compound is a compound of Formula IA.
Aspect 85. The compound according to aspect 83, or a pharmaceutically acceptable salt thereof, wherein the compound is a compound of Formula IB.
Aspect 86. The compound according to aspect 84 or aspect 85, or a pharmaceutically acceptable salt thereof, wherein X is CH.
Aspect 87. The compound according to aspect 84 or aspect 85, or a pharmaceutically acceptable salt thereof, wherein X is N.
Aspect 88. The compound according to any one of aspects 83-87, or a pharmaceutically acceptable salt thereof, wherein Cy is optionally substituted C₅-C₇ cycloalkyl.
Aspect 89. The compound according to aspect 88, or a pharmaceutically acceptable salt thereof, wherein Cy is unsubstituted C₅-C₇ cycloalkyl.
Aspect 90. The compound according to aspect 88, or a pharmaceutically acceptable salt thereof, wherein Cy is C₅-C₇ cycloalkyl substituted with one or more of OH, deuterium, halo, cyano, C₁-C₃alkyl, C₁-C₃haloalkyl, C₁-C₃alkoxy, deuterated C₁-C₃alkoxy, C₁-C₃haloalkoxy, NH₂, NH(C₁-C₆alkyl), or N(C₁-C₆alkyl)₂.
Aspect 91. The compound according to aspect 90, or a pharmaceutically acceptable salt thereof, wherein Cy is C₅-C₇ cycloalkyl substituted with OH, C₁-C₃alkoxy, or cyano.
Aspect 92. The compound according to aspect 88, or a pharmaceutically acceptable salt thereof, wherein the optionally substituted C₅-C₇ cycloalkyl is an optionally substituted cyclohexyl or an optionally substituted cycloheptyl.
Aspect 93. The compound according to aspect 88, or a pharmaceutically acceptable salt thereof, wherein the optionally substituted C₅-C₇ cycloalkyl is an optionally substituted cyclopentyl.
Aspect 94. The compound according to aspect 93, or a pharmaceutically acceptable salt thereof, wherein the optionally substituted cyclopentyl is an unsubstituted cyclopentyl.
Aspect 95. The compound according to aspect 88, or a pharmaceutically acceptable salt thereof, wherein the optionally substituted C₅-C₇ cycloalkyl is an optionally substituted cyclohexyl.
Aspect 96. The compound according to aspect 95, or a pharmaceutically acceptable salt thereof, wherein the cyclohexyl is substituted with one or more OH, deuterium, halo, cyano, C₁-C₃alkyl, C₁-C₃haloalkyl, C₁-C₃alkoxy, deuterated C₁-C₃alkoxy, C₁-C₃haloalkoxy, NH₂, NH(C₁-C₆alkyl), or N(C₁-C₆alkyl)₂.
Aspect 97. The compound according to aspect 96, or a pharmaceutically acceptable salt thereof, wherein the cyclohexyl is substituted with OH, C₁-C₃alkoxy, or cyano.
Aspect 98. The compound according to aspect 95, or a pharmaceutically acceptable salt thereof, wherein the optionally substituted cyclohexyl is an unsubstituted cyclohexyl.
Aspect 99. The compound according to aspect 88, or a pharmaceutically acceptable salt thereof, wherein the optionally substituted C₅-C₇ cycloalkyl is an optionally substituted cycloheptyl.
Aspect 100. The compound according to aspect 99, or a pharmaceutically acceptable salt thereof, wherein the optionally substituted cycloheptyl is an unsubstituted cycloheptyl.
Aspect 101. The compound according to any one of aspects 83-87, or a pharmaceutically acceptable salt thereof, wherein Cy is an optionally substituted 6-membered heterocycloalkyl.
Aspect 102. The compound according to aspect 101, or a pharmaceutically acceptable salt thereof, wherein Cy is an optionally substituted piperidinyl, an optionally substituted tetrahydropyranyl, an optionally substituted thianyl, an optionally substituted thianyl sulfone, an optionally substituted thianyl sulfoxide, or an optionally substituted 1-oxo-1-imino-1-thiacyclohexyl.
Aspect 103. The compound according to any one of aspects 101-102, or a pharmaceutically acceptable salt thereof, wherein Cy is an optionally substituted piperidinyl.
Aspect 104. The compound according to any one of aspects 101-102, or a pharmaceutically acceptable salt thereof, wherein Cy is an optionally substituted tetrahydropyranyl.
Aspect 105. The compound according to any one of aspects 83-87, or a pharmaceutically acceptable salt thereof, wherein Cy is an optionally substituted 7-membered heterocycloalkyl.
Aspect 106. The compound according to aspect 105, or a pharmaceutically acceptable salt thereof, wherein the optionally substituted 7-membered heterocycloalkyl is an optionally substituted oxazepanyl, an optionally substituted thiepanyl, an optionally substituted thiepanyl-1-oxide, an optionally substituted thiepanyl-1,1-dioxide, an optionally substituted azepanyl, or an optionally substituted 1-imino-1-oxide-thiepanyl.
Aspect 107. The compound according to any one of the preceding aspects, or a pharmaceutically acceptable salt thereof, wherein R¹ is optionally substituted heteroaryl.
Aspect 108. The compound according to aspect 107, or a pharmaceutically acceptable salt thereof, wherein R¹ is optionally substituted 9-membered heteroaryl, such as optionally substituted benzofuranyl, optionally substituted isobenzofuranyl, optionally substituted benzoxazolyl, optionally substituted benzoisoxazolyl, optionally substituted benzothiazolyl, optionally substituted benzoisothiazolyl, optionally substituted indolyl, optionally substituted isoindolyl, optionally substituted indazolyl, optionally substituted benzimidazolyl, or optionally substituted benzothiophenyl.
Aspect 109. The compound according to aspect 107, or a pharmaceutically acceptable salt thereof, wherein R¹ is optionally substituted 5- or 6-membered heteroaryl.
Aspect 110. The compound according to aspect 109, or a pharmaceutically acceptable salt thereof, wherein R¹ is optionally substituted 5-membered heteroaryl.
Aspect 111. The compound according to aspect 110, or a pharmaceutically acceptable salt thereof, wherein the 5-membered heteroaryl is optionally substituted oxadiazolyl, optionally substituted thiadiazolyl, optionally substituted oxazolyl, optionally substituted pyrazolyl, optionally substituted imidazolyl, optionally substituted triazolyl, optionally substituted tetrazolyl, or optionally substituted thiazolyl.
Aspect 112. The compound according to aspect 111, or a pharmaceutically acceptable salt thereof, wherein the 5-membered heteroaryl is optionally substituted with one or more of oxo, NR^{b}R^{c}, C₁-C₄alkyl, C₁-C₄haloalkyl, C₁-C₆hydroxyalkyl, halo, or C(O)OC₁-C₄alkyl; and wherein each R^{b} and R^{c} is independently hydrogen or C₁-C₄alkyl.
Aspect 113. The compound according to aspect 109, or a pharmaceutically acceptable salt thereof, wherein R¹ is optionally substituted 6-membered heteroaryl.
Aspect 114. The compound according to aspect 113, or a pharmaceutically acceptable salt thereof, wherein the optionally substituted 6-membered heteroaryl is optionally substituted pyridinyl, optionally substituted pyrimidinyl, or optionally substituted pyridazinyl.
Aspect 115. The compound according to aspect 114, or a pharmaceutically acceptable salt thereof, wherein the 6-membered heteroaryl is optionally substituted with one or more of NR^{b}R^{c}, C₁-C₄alkyl, C₁-C₄alkoxy, C₁-C₆hydroxyalkyl, cyano, or halo; and wherein each R^{b} and R^{c} is independently hydrogen or C₁-C₄alkyl.
Aspect 116. The compound according to any one of aspects 83-106, or a pharmaceutically acceptable salt thereof, wherein R¹ is optionally substituted aryl.
Aspect 117. The compound according to aspect 116, or a pharmaceutically acceptable salt thereof, wherein the optionally substituted aryl is phenyl, optionally substituted with C₁-C₆alkyl, C₁-C₆haloalkyl, C₁-C₆hydroxyalkyl, halo, or cyano.
Aspect 118. The compound according to any one of aspects 83-106, or a pharmaceutically acceptable salt thereof, wherein R¹ is optionally substituted 4-, 5- or 6-membered heterocycloalkyl.
Aspect 119. The compound according to aspect 118, or a pharmaceutically acceptable salt thereof, wherein R¹ is optionally substituted imidazolidinonyl, optionally substituted pyrrolidinyl, or optionally substituted tetrahydrofuranyl.
Aspect 120. The compound according to aspect 119, or a pharmaceutically acceptable salt thereof, wherein R¹ is pyrrolidinyl, optionally substituted with one or more of oxo, C₁-C₆hydroxyalkyl, C₁-C₆alkyl, C₁-C₆alkoxy(alkylene), C₁-C₆cyanoalkyl, OH, cyano, CONR^{b}R^{c}, and wherein each R^{b} and R^{c} is independently hydrogen or C₁-C₄alkyl.
Aspect 121. The compound according to any one of the preceding aspects, or a pharmaceutically acceptable salt thereof, wherein L is a bond.
Aspect 122. The compound according to any one of aspects 83-121, or a pharmaceutically acceptable salt thereof, wherein L is —O—.
Aspect 123. The compound according to any one of aspects 83-121, or a pharmaceutically acceptable salt thereof, wherein L is —NR^{a}—, wherein R^{a} is hydrogen or C₁-C₄alkyl.
Aspect 124. The compound according to any one of aspects 83-121, or a pharmaceutically acceptable salt thereof, wherein L is optionally substituted C₁-C₄alkylene.
Aspect 125. The compound according to aspect 124, or a pharmaceutically acceptable salt thereof, wherein L is C₁-C₄alkylene substituted with deuterium, oxo, halo, NH₂, OH, C₁-C₃alkoxy, NR^{h}CO(C₁-C₄alkyl), NR^{h}C(═O)O(C₁-C₄alkyl), or an optionally substituted 4-, 5-, or 6-membered heterocycloalkyl containing a nitrogen wherein the nitrogen is connected to the alkylene carbon and each R^{h} is independently hydrogen or C₁-C₄alkyl.
Aspect 126. The compound according to any one of the preceding aspects, or a pharmaceutically acceptable salt thereof, wherein R² is optionally substituted phenyl.
Aspect 127. The compound according to any one of aspects 83-125, or a pharmaceutically acceptable salt thereof, wherein R² is optionally substituted pyridinyl.
Aspect 128. The compound according to any one of aspects 83-125, or a pharmaceutically acceptable salt thereof, wherein R² is optionally substituted 5-membered heteroaryl.
Aspect 129. The compound according to aspect 128, or a pharmaceutically acceptable salt thereof, wherein the optionally substituted 5-membered heteroaryl is optionally substituted triazolyl, optionally substituted pyrazolyl, optionally substituted oxazolyl, or optionally substituted isoxazolyl.
Aspect 130. The compound according to aspect 129, or a pharmaceutically acceptable salt thereof, wherein R² is

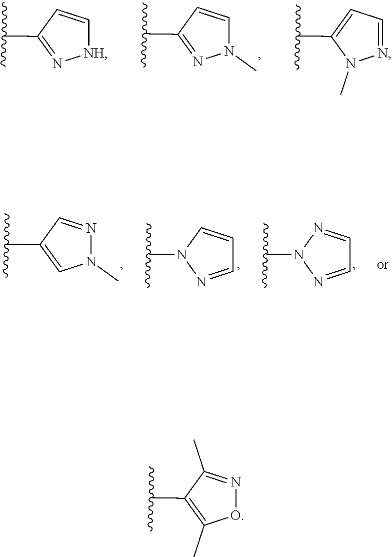

Aspect 131. The compound according to any one of aspects 83-125, or a pharmaceutically acceptable salt thereof, wherein R² is optionally substituted heterocycloalkyl.
Aspect 132. The compound according to aspect 131, or a pharmaceutically acceptable salt thereof, wherein the optionally substituted heterocycloalkyl is optionally substituted azetidinyl, optionally substituted pyrrolidinyl, optionally substituted tetrahydropyranyl, optionally substituted piperidinyl, optionally substituted morpholinyl, or optionally substituted dioxanyl.
Aspect 133. The compound according to aspect 132, or a pharmaceutically acceptable salt thereof, wherein R² is

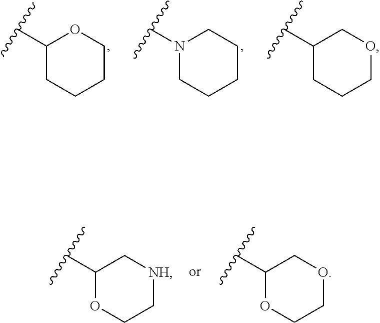 [SMILES: *C1CCCCO1 ; *N1CCCCC1 ; *C1CCCOC1 ; *C1CNCCO1 ; *C1COCCO1]

Aspect 134. The compound according to any one of aspects 83-125, or a pharmaceutically acceptable salt thereof, wherein R² is optionally substituted C₁-C₆alkyl.
Aspect 135. The compound according to aspect 134, or a pharmaceutically acceptable salt thereof, wherein the C₁-C₆alkyl is CH₃, CH(CH₃)₂, CH₂CH(CH₃)₂, C(CH₃)₃, or CH₂C(CH₃)₃.
Aspect 136. The compound according to any one of aspects 83-125, or a pharmaceutically acceptable salt thereof, wherein R² is optionally substituted C₁-C₆alkoxy.
Aspect 137. The compound according to aspect 136, or a pharmaceutically acceptable salt thereof, wherein the C₁-C₆alkoxy is OCH₃, or OCH(CH₃)₂.
Aspect 138. The compound according to any one of aspects 83-125, or a pharmaceutically acceptable salt thereof, wherein R² is optionally substituted C₁-C₆hydroxyalkyl.
Aspect 139. The compound according to aspect 138, or a pharmaceutically acceptable salt thereof, wherein the C₁-C₆hydroxyalkyl is C(OH)(CH₃)₂.
Aspect 140. The compound according to any one of aspects 83-125, or a pharmaceutically acceptable salt thereof, wherein R² is optionally substituted C₁-C₆haloalkyl.
Aspect 141. The compound according to aspect 140, or a pharmaceutically acceptable salt thereof, wherein the C₁-C₆haloalkyl is CF₃, CH(CH₃)(CF₃), CH₂CF₃, C(CH₃)₂F, CH₂C(CH₃)₂F, C(CH₃)F₂, CH₂C(CH₃)F₂, CHFCH(CH₃)₂, or CF₂CH(CH₃)₂.
Aspect 142. The compound according to any one of aspects 83-125, or a pharmaceutically acceptable salt thereof, wherein R² is optionally substituted C₁-C₆haloalkoxy.
Aspect 143. The compound according to aspect 142, or a pharmaceutically acceptable salt thereof, wherein the C₁-C₆haloalkoxy is OCF₃, OCH₂CF₃, OCHF₂, OCH₂F, or OCH(CH₃)(CF₃).
Aspect 144. The compound according to any one of aspects 83-125, or a pharmaceutically acceptable salt thereof, wherein R² is optionally substituted C₃-C₆cycloalkyl.
Aspect 145. The compound according to aspect 144, or a pharmaceutically acceptable salt thereof, wherein the C₃-C₆cycloalkyl is optionally substituted with one or more of C₁-C₄alkyl or C₁-C₄haloalkyl.
Aspect 146. The compound according to any of aspects 83-125, or a pharmaceutically acceptable salt thereof, wherein R² is NR⁹.
Aspect 147. The compound according to any one of the preceding aspects, or a pharmaceutically acceptable salt thereof, wherein Ar¹ is an optionally substituted thiophenyl or an optionally substituted thiazolyl.
Aspect 148. The compound according to any one of aspects 83-84, 86-92, 95-96, or 107-147, wherein the compound of Formula IA is a compound of Formula IA-1:

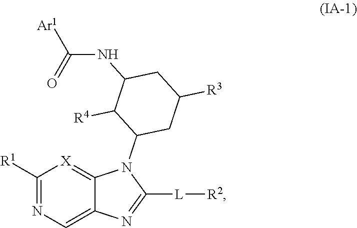

or a pharmaceutically acceptable salt thereof, wherein R³ is hydrogen, OH, or C₁-C₃alkoxy; R⁴ is hydrogen, OH, or C₁-C₃alkoxy, and wherein at least one of R³ and R⁴ is hydrogen.
Aspect 149. The compound according to aspect 148, wherein the compound of Formula IA-1 is a compound of Formula IA-1a:

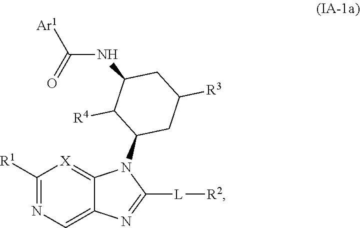

or a pharmaceutically acceptable salt thereof.
Aspect 150. The compound according to aspect 148 or 149, or a pharmaceutically acceptable salt thereof, wherein the C₁-C₃alkoxy is OCH₃ or OCH₂CH₃.
Aspect 151. The compound according to any one of aspects 148-150, or a pharmaceutically acceptable salt thereof, wherein R⁴ is hydrogen.
Aspect 152. The compound according to any one of aspects 148-150, or a pharmaceutically acceptable salt thereof, wherein R³ is hydrogen.
Aspect 153. The compound according to any one of aspects 83-84, 86-92, 99, or 107-147, wherein the compound of Formula IA is a compound of Formula IA-2 or Formula IA-3:

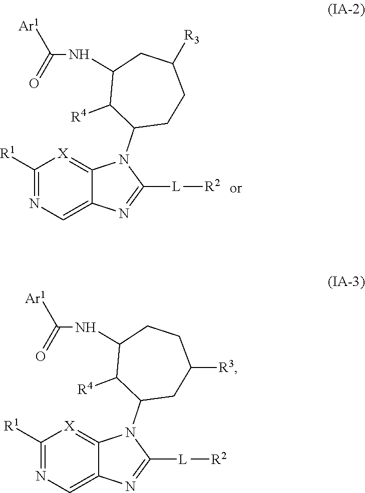

or a pharmaceutically acceptable salt thereof, wherein R³ is hydrogen, OH, or C₁-C₃alkoxy; R⁴ is hydrogen, OH, or C₁-C₃alkoxy, and wherein at least one of R³ and R⁴ is hydrogen.
Aspect 154. The compound according to aspect 153, wherein the compound of Formula IA-2 is a compound of Formula IA-2a, or a pharmaceutically acceptable salt thereof, and the compound of Formula IA-3 is a compound of Formula IA-3a, or a pharmaceutically acceptable salt thereof:

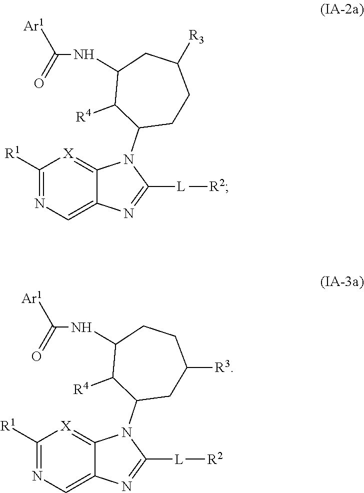

Aspect 155. The compound according to any one of aspects 83-84, 86-87, 101-104, and 107-147, wherein the compound of Formula IA is a compound of Formula IA-4:

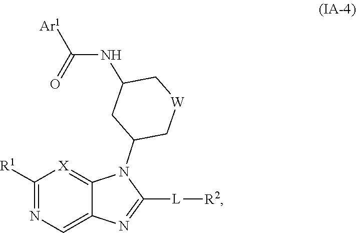

or a pharmaceutically acceptable salt thereof, wherein W is N—R⁵, S, SO, SO₂, or O; and R⁵ is hydrogen, C₁-C₆alkyl, C(O)C₁-C₆alkyl, ═O, OH, or C₁-C₆alkoxy.
Aspect 156. The compound according to aspect 155, wherein the compound of Formula IA is a compound of Formula IA-4a:

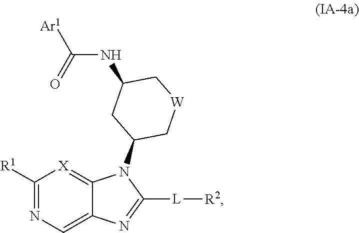

or a pharmaceutically acceptable salt thereof.
Aspect 157. The compound according to aspect 155 or aspect 156, or a pharmaceutically acceptable salt thereof, wherein W is N—R⁵.
Aspect 158. The compound according to aspect 155 or aspect 156, or a pharmaceutically acceptable salt thereof, wherein W is O.
Aspect 159. The compound according to any one of aspects 83-84, and 86-147, wherein the compound of Formula IA is a compound of Formula IA-5:

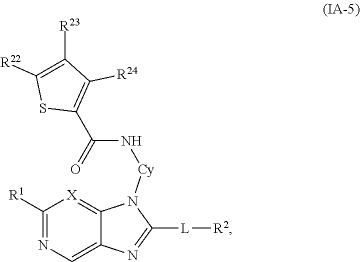

or a pharmaceutically acceptable salt thereof, wherein R²² is halo or optionally substituted C₂-C₆alkynyl; and R²³ and R²⁴ are hydrogen.
Aspect 160. The compound according to any one of aspects 83-84, and 86-147, wherein the compound of Formula IA is a compound of Formula IA-6:

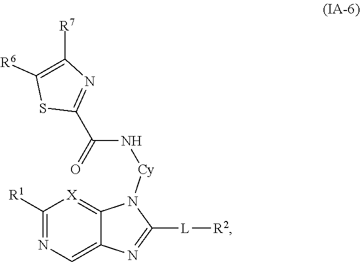

or a pharmaceutically acceptable salt thereof, wherein R⁶ is halo, C₁-C₆haloalkyl, cyano, or optionally substituted C₂-C₆alkynyl; and R⁷ is hydrogen.
Aspect 161. The compound according to any one of aspects 83-84, 86-121, 126, and 147-160, wherein the compound of Formula IA is a compound of Formula IA-7:

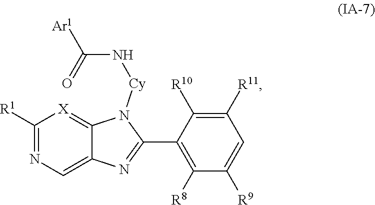

or a pharmaceutically acceptable salt thereof, wherein R⁸, R⁹, R¹⁰, and R¹¹ are each independently hydrogen, halo, C₁-C₄alkyl, C₃-C₄cycloalkyl, C₁-C₄alkoxy, cyano, OH, NR^{b}R^{c}, C(O)NR^{b}R^{c}, C(O)OC₁-C₄alkyl, COOH, or C₁₋₄alkylenecarboxylic acid; and
   each R^{b} and each R^{c} is independently hydrogen or C₁-C₄alkyl.
Aspect 162. The compound according to aspect 161, or a pharmaceutically acceptable salt thereof, wherein at least two of R⁸, R⁹, R¹⁰, and R¹¹ are hydrogen.
Aspect 163. The compound according to aspect 161, or a pharmaceutically acceptable salt thereof, wherein at least three of R⁸, R⁹, R¹⁰, and R¹¹ are hydrogen.
Aspect 164. The compound according to any one of aspects 161-163, or a pharmaceutically acceptable salt thereof, wherein at least one of R⁸, R⁹, R¹⁰, and R¹¹ is F, CH₃, OCH₃, NH₂, or cyano.
Aspect 165. The compound according to any one of aspects 83-84, 86-121, 127, and 147-160, wherein the compound of Formula IA is a compound of Formula IA-8:

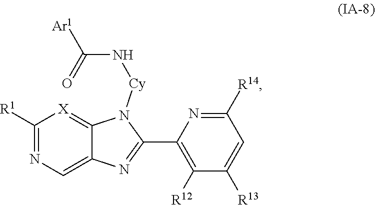

or a pharmaceutically acceptable salt thereof, wherein R¹², R¹³, and R¹⁴ are each independently hydrogen, halo, C₁-C₄alkyl, C₃-C₄cycloalkyl, C₁-C₄alkoxy, cyano, OH, NR^{b}R^{c}, C(O)NR^{b}R^{c}, C(O)OC₁-C₄alkyl, COOH, or C₁₋₄alkylenecarboxylic acid; and
   each R^{b} and each R^{c} is independently hydrogen or C₁-C₄alkyl.
Aspect 166. The compound according to aspect 165, or a pharmaceutically acceptable salt thereof, wherein at least two of R¹², R¹³, and R¹⁴ are hydrogen.
Aspect 167. The compound according to aspect 165 or aspect 166, or a pharmaceutically acceptable salt thereof, wherein at least one of R¹², R¹³, and R¹⁴ is F, CH₃, OCH₃, NH₂, or cyano.
Aspect 168. The compound according to any one of aspects 83-84, 86-107, 109-111, and 121-167, wherein the compound of Formula IA is a compound of Formula IA-9:

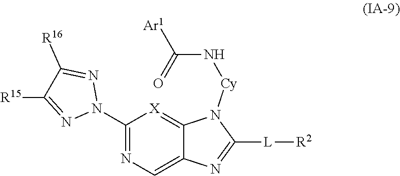

or a pharmaceutically acceptable salt thereof, wherein R¹⁵ and R¹⁶ are each independently hydrogen, halo, C₁-C₆alkyl, C₁-C₆hydroxyalkyl, C(O)OC₁-C₆alkyl, or CONR^{b}R^{c}; and
   each R^{b} and each R^{c} is independently hydrogen or C₁-C₄alkyl.
Aspect 169. The compound according to aspect 168, or a pharmaceutically acceptable salt thereof, wherein at least one of R¹⁵ and R¹⁶ is hydrogen.
Aspect 170. The compound according to aspect 168 or aspect 169, or a pharmaceutically acceptable salt thereof, wherein one of R¹⁵ and R¹⁶ is CH₃, C(O)OCH₃, or C(CH₃)₂OH.
Aspect 171. The compound according to any one of aspects 83-84, 86-107, 109-111, and 121-167, wherein the compound of Formula IA is a compound of Formula IA-10:

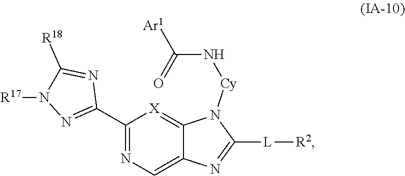

or a pharmaceutically acceptable salt thereof, wherein R¹⁷ is hydrogen or C₁-C₆alkyl;
R¹⁸ is hydrogen, halo, C₁-C₆alkyl, C(O)OC₁-C₆alkyl, or CONR^{b}R^{c}; and each R^{b} and each R^{c} is independently hydrogen or C₁-C₄alkyl.
Aspect 172. The compound according to any one of aspects 83-84, 86-107, 109-111, and 121-167, wherein the compound of Formula IA is a compound of Formula IA-11:

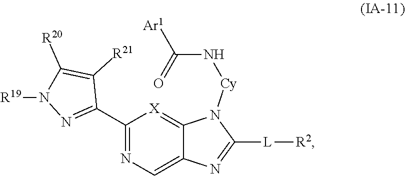

or a pharmaceutically acceptable salt thereof, wherein R¹⁹ is hydrogen, C₁-C₆alkyl, or C₁-C₆haloalkyl;
and R²⁰ and R²¹ are each independently hydrogen, halo, C₁-C₆alkyl, C₁-C₆haloalkyl, C(O)OC₁-C₆alkyl, or CONR^{b}R^{c}; and each R^{b} and each R^{c} is independently hydrogen or C₁-C₄alkyl.
Aspect 173. The compound according to any one of aspects 83, 85-92, 95, 98 or 107-147, wherein the compound of Formula IB is a compound of Formula IB-1:

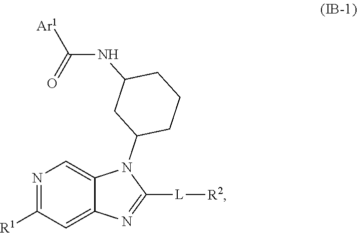

or a pharmaceutically acceptable salt thereof.
Aspect 174. The compound according to aspect 173, wherein the compound of Formula IB-1 is a compound of Formula IB-1a:

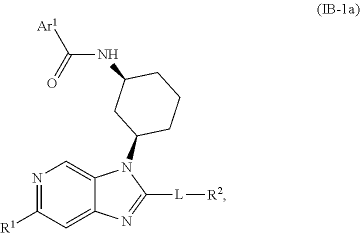

or a pharmaceutically acceptable salt thereof.
Aspect 175. The compound according to aspect 173 or aspect 174, or a pharmaceutically acceptable salt thereof, wherein R¹ is optionally substituted triazolyl, optionally substituted oxadiazolyl, or optionally substituted pyrazolyl.
Aspect 176. The compound according to any one of aspects 173-175, or a pharmaceutically acceptable salt thereof, wherein L is a bond and R² is optionally substituted phenyl or optionally substituted pyridinyl.
Aspect 177. The compound according to any one of aspects 173-176, or a pharmaceutically acceptable salt thereof, wherein Ar¹ is optionally substituted thiophenyl or optionally substituted thiazolyl.
Aspect 178. A compound, wherein said compound is a compound of Formula IIA or Formula IIB:

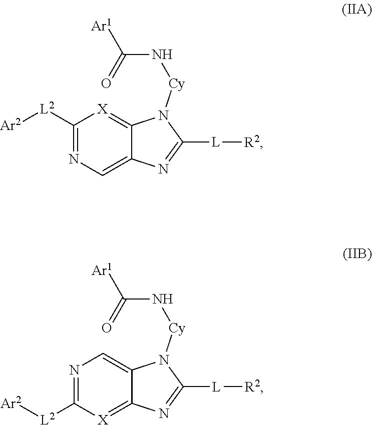

or a pharmaceutically acceptable salt thereof, wherein:
X is CH or N;
Cy is optionally substituted C₅-C₇ cycloalkyl or optionally substituted 6- or 7-membered heterocycloalkyl;
L is a bond, —O—, —NR^{a}—, or optionally substituted C₁-C₄alkylene;
L² is —O—, —NR^{a}—, or —CH₂—; each R^{a} is independently hydrogen or C₁-C₄alkyl;
R² is optionally substituted phenyl, optionally substituted pyridinyl, optionally substituted 5-membered heteroaryl, optionally substituted heterocycloalkyl, optionally substituted C₁-C₆alkyl, optionally substituted C₁-C₆alkoxy, optionally substituted C₁-C₆hydroxyalkyl, optionally substituted C₁-C₆haloalkyl, optionally substituted C₁-C₆haloalkoxy, optionally substituted C₃-C₆cycloalkyl, or NR^{f}R^{g};
R^{f} and R^{g} are each independently hydrogen, C₁-C₆alkyl, C₁-C₆haloalkyl, OH, C₁-C₄alkoxy, C₁-C₆hydroxyalkyl, or C₃-C₆cycloalkyl;
Ar¹ is an optionally substituted five-membered heteroaryl; and
Ar² is optionally substituted heteroaryl, optionally substituted aryl, or optionally substituted 4-, 5- or 6-membered heterocycloalkyl.
Aspect 179. The compound according to aspect 178, or a pharmaceutically acceptable salt thereof, wherein X is CH.
Aspect 180. The compound according to aspect 178 or aspect 179, or a pharmaceutically acceptable salt thereof, wherein L² is —O—.
Aspect 181. The compound according to any one of aspects 178-180, or a pharmaceutically acceptable salt thereof, wherein Ar² is optionally substituted 5- or 6-membered heteroaryl or optionally substituted 4-, 5- or 6-membered heterocycloalkyl.
Aspect 182. The compound according to aspect 181, or a pharmaceutically acceptable salt thereof, where Ar² is optionally substituted tetrahydrofuranyl or optionally substituted pyrazolyl.
Aspect 183. The compound according to any one of aspects 178-182 or a pharmaceutically acceptable salt thereof, wherein L is a bond and R² is optionally substituted phenyl or optionally substituted pyridinyl.
Aspect 184. The compound according to any one of aspects 178-183, or a pharmaceutically acceptable salt thereof, wherein Ar¹ is optionally substituted thiophenyl or optionally substituted thiazolyl.
Aspect 185. A compound, wherein said compound is a compound of Formula IIIA, Formula IIIB, Formula IIIC, or Formula IIID:

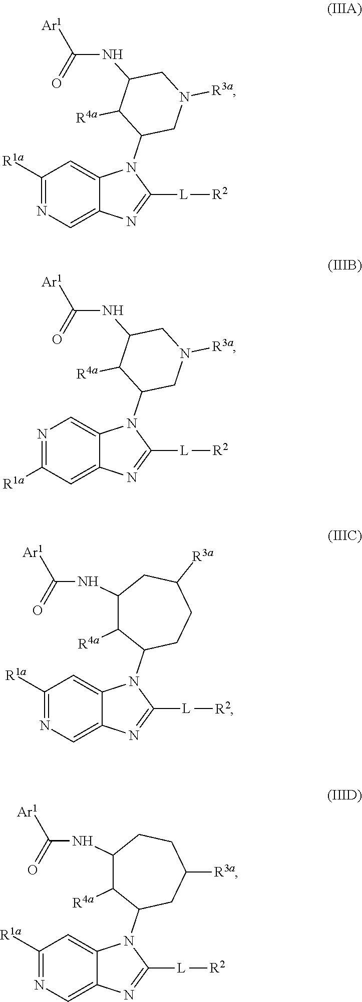

or a pharmaceutically acceptable salt thereof, wherein:
L is a bond, —O—, —NR^{a}—, or optionally substituted C₁-C₄alkylene;
   R^{a} is hydrogen or C₁-C₄alkyl;
R^{1a} is hydrogen, C₁-C₆alkyl, C₁-C₆alkoxy, halo, C₁-C₆haloalkyl, C₁-C₆haloalkoxy, CONR^{j}(C₁-C₄alkyl), CONR^{J}(C₃-C₄cycloalkyl), or NR^{b}R^{c};
   R^{b} and R^{c} are each independently hydrogen or C₁-C₄alkyl;
   each R^{j} is independently hydrogen or C₁-C₄alkyl;
R² is optionally substituted phenyl, optionally substituted pyridinyl, optionally substituted 5-membered heteroaryl, optionally substituted heterocycloalkyl, optionally substituted C₁-C₆alkyl, optionally substituted C₁-C₆alkoxy, optionally substituted C₁-C₆hydroxyalkyl, optionally substituted C₁-C₆haloalkyl, optionally substituted C₁-C₆haloalkoxy, optionally substituted C₃-C₆cycloalkyl, or NR^{f}R^{g};
R^{f} and R^{g} are each independently hydrogen, C₁-C₆alkyl, C₁-C₆haloalkyl, OH, C₁-C₄alkoxy, C₁-C₆hydroxyalkyl, or C₃-C₆cycloalkyl
one of R^{3a} or R^{4a} is hydrogen, deuterium, OH, cyano, C₁-C₃alkyl, C₁-C₃haloalkyl, C₁-C₃alkoxy, C₁-C₃haloalkoxy, deuterated C₁-C₃alkoxy, NH₂, NH(C₁-C₄alkyl), or N(C₁-C₄alkyl)₂; and
the other of R^{3a} or R^{4a} is OH, cyano, C₁-C₃alkyl, C₁-C₃haloalkyl, C₁-C₃alkoxy, C₁-C₃haloalkoxy, deuterated C₁-C₃alkoxy, NH₂, NH(C₁-C₄alkyl), or N(C₁-C₄alkyl)₂; and
Ar¹ is an optionally substituted five-membered heteroaryl.
Aspect 186. The compound according to aspect 185, or a pharmaceutically acceptable salt thereof, wherein the compound is a compound of Formula IIIA or Formula IIIB.
Aspect 187. The compound according to aspect 186, wherein the compound of Formula IIIA is a compound of Formula IIIA-1, or a pharmaceutically acceptable salt thereof, and the compound of Formula IIIB is a compound of Formula IIIB-1, or a pharmaceutically acceptable salt thereof:

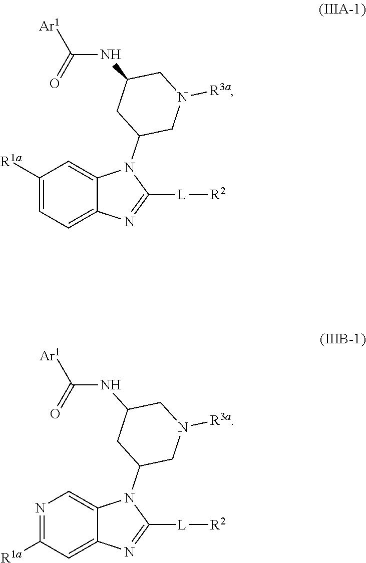

Aspect 188. The compound according to aspect 185, or a pharmaceutically acceptable salt thereof, wherein the compound is a compound of Formula IIIC or Formula IIID.
Aspect 189. The compound according to aspect 188, wherein the compound of Formula IIIC is a compound of Formula IIIC-1, or a pharmaceutically acceptable salt thereof, and the compound of Formula IIID is a compound of Formula IIID-1, or a pharmaceutically acceptable salt thereof:

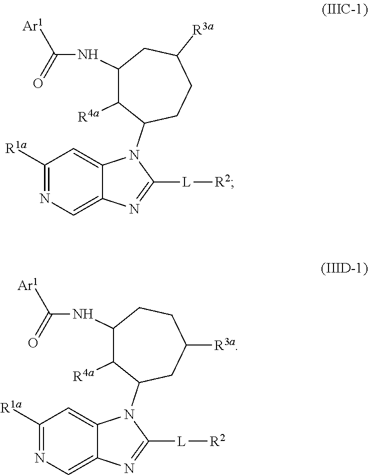

Aspect 190. The compound according to any one of aspects 185-189, or a pharmaceutically acceptable salt thereof, wherein one of R^{3a} or R^{4a} is OH or C₁-C₃alkoxy; and the other of R^{3a} or R^{4a} is hydrogen.
Aspect 191. The compound according to any one of aspects 185-190, or a pharmaceutically acceptable salt thereof, wherein L is a bond and R² is optionally substituted phenyl or optionally substituted pyridinyl.
Aspect 192. The compound according to any one of aspects 185-191, or a pharmaceutically acceptable salt thereof, wherein Ar¹ is optionally substituted thiophenyl or optionally substituted thiazolyl.
Aspect 193. The compound according to any one of aspects 185-192, or a pharmaceutically acceptable salt thereof, wherein R^{1a} is hydrogen, NH₂, NHCH₃, F, OCH₃, or C(O)NH(CH₃).
Aspect 194. A compound, wherein said compound is a compound of Formula IVA or a compound of Formula IVB:

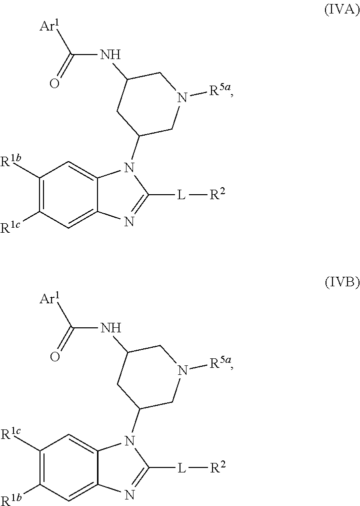

or a pharmaceutically acceptable salt thereof, wherein:
L is a bond, —O—, —NR^{a}—, or optionally substituted C₁-C₄alkylene;
   R^{a} is hydrogen or C₁-C₄alkyl;
R^{1b} is optionally substituted heteroaryl, optionally substituted aryl, optionally substituted 4-, 5- or 6-membered heterocycloalkyl, CONR^{j}(C₁-C₄alkyl), CONR^{j}(C₃-C₄cycloalkyl), or cyano;
   each R^{j} is independently hydrogen or C₁-C₄alkyl;
R^{1c} is hydrogen, halo, C₁-C₆alkyl, C₁-C₆haloalkyl, C₁-C₆alkoxy, or C₁-C₆haloalkoxy;
R² is optionally substituted phenyl, optionally substituted pyridinyl, optionally substituted 5-membered heteroaryl, optionally substituted heterocycloalkyl, optionally substituted C₁-C₆alkyl, optionally substituted C₁-C₆alkoxy, optionally substituted C₁-C₆hydroxyalkyl, optionally substituted C₁-C₆haloalkyl, optionally substituted C₁-C₆haloalkoxy, optionally substituted C₃-C₆cycloalkyl, or NR^{f}R^{g};
R^{f} and R^{g} are each independently hydrogen, C₁-C₆alkyl, C₁-C₆haloalkyl, OH, C₁-C₄alkoxy, C₁-C₆hydroxyalkyl, or C₃-C₆cycloalkyl;
R^{5a} is hydrogen, C₁-C₆alkyl, C(O)C₁-C₆alkyl, C(O)NR^{b}R^{c}, oxo, OH, or C₁-C₆alkoxy;
each R^{b} and each R^{c} is independently hydrogen or C₁-C₄alkyl; and
Ar¹ is an optionally substituted five-membered heteroaryl.
Aspect 195. The compound according to aspect 194, wherein the compound of Formula IVA is a compound of Formula IVA-1, or a pharmaceutically acceptable salt thereof, and the compound of Formula IVB is a compound of Formula IVB-1, or a pharmaceutically acceptable salt thereof:

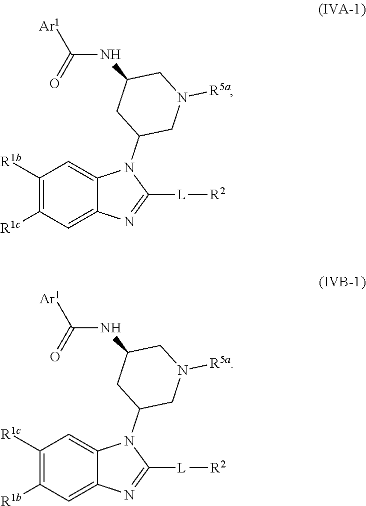

Aspect 196. The compound according to aspect 194 or aspect 195, or a pharmaceutically acceptable salt thereof, wherein L is a bond and R² is optionally substituted phenyl or optionally substituted pyridinyl.
Aspect 197. The compound according to any one of aspects 194-196, or a pharmaceutically acceptable salt thereof, wherein Ar¹ is optionally substituted thiophenyl or optionally substituted thiazolyl.
Aspect 198. The compound according to any of aspects 194-197, or a pharmaceutically acceptable salt thereof, wherein R^{1b} is optionally substituted 5- or 6-membered heteroaryl.
Aspect 199. The compound according to any of aspects 194-198, or a pharmaceutically acceptable salt thereof, wherein R^{1b} is CONH(CH₃) or cyano.
Aspect 200. The compound according to any of aspects 194-199, or a pharmaceutically acceptable salt thereof, wherein R^{1c} is hydrogen or OCH₃.
Aspect 201. The compound according to any of aspects 194-200, or a pharmaceutically acceptable salt thereof, wherein R^{5a} is C(O)CH₃, CH₃, or C(O)NH₂.
Aspect 202. A compound, wherein said compound is a compound of Formula VA, Formula VB, Formula VC, or Formula VD:

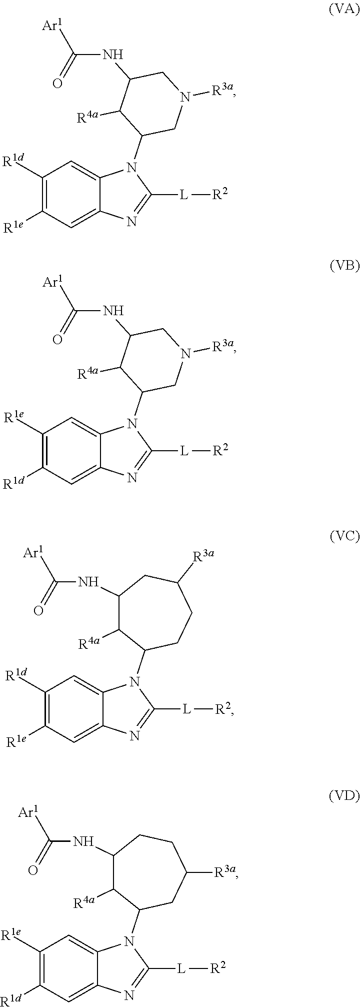

or a pharmaceutically acceptable salt thereof, wherein:
L is a bond, —O—, —NR^{a}—, or optionally substituted C₁-C₄alkylene;
   R^{a} is hydrogen or C₁-C₄alkyl;
R^{1d} is optionally substituted heteroaryl, optionally substituted aryl, optionally substituted 4-, 5- or 6-membered heterocycloalkyl, CONR^{j}(C₁-C₄alkyl), CONR^{j}(C₃-C₄cycloalkyl), or cyano;
   each R^{j} is independently hydrogen or C₁-C₄alkyl;
R^{1e} is hydrogen, halo, C₁-C₆alkyl, C₁-C₆haloalkyl, C₁-C₆alkoxy, or C₁-C₆haloalkoxy;
R² is optionally substituted phenyl, optionally substituted pyridinyl, optionally substituted 5-membered heteroaryl, optionally substituted heterocycloalkyl, optionally substituted C₁-C₆alkyl, optionally substituted C₁-C₆alkoxy, optionally substituted C₁-C₆hydroxyalkyl, optionally substituted C₁-C₆haloalkyl, optionally substituted C₁-C₆haloalkoxy, optionally substituted C₃-C₆cycloalkyl, or NR^{f}R^{g};
R^{f} and R^{g} are each independently hydrogen, C₁-C₆alkyl, C₁-C₆haloalkyl, OH, C₁-C₄alkoxy, C₁-C₆hydroxyalkyl, or C₃-C₆cycloalkyl;
one of R^{3a} or R^{4a} is hydrogen, deuterium, OH, cyano, C₁-C₃alkyl, C₁-C₃haloalkyl, C₁-C₃alkoxy, C₁-C₃haloalkoxy, deuterated C₁-C₃alkoxy, NH₂, NH(C₁-C₄alkyl), or N(C₁-C₄alkyl)₂; and
the other of R^{3a} or R^{4a} is OH, cyano, C₁-C₃alkyl, C₁-C₃haloalkyl, C₁-C₃alkoxy, C₁-C₃haloalkoxy, deuterated C₁-C₃alkoxy, NH₂, NH(C₁-C₄alkyl), or N(C₁-C₄alkyl)₂; and
Ar¹ is an optionally substituted five-membered heteroaryl.
Aspect 203. The compound according to aspect 202, or a pharmaceutically acceptable salt thereof, wherein the compound is a compound of Formula VA or Formula VB.
Aspect 204. The compound according to aspect 203, wherein the compound of Formula VA is a compound of Formula VA-1, or a pharmaceutically acceptable salt thereof, and the compound of Formula VB is a compound of Formula VB-1, or a pharmaceutically acceptable salt thereof:

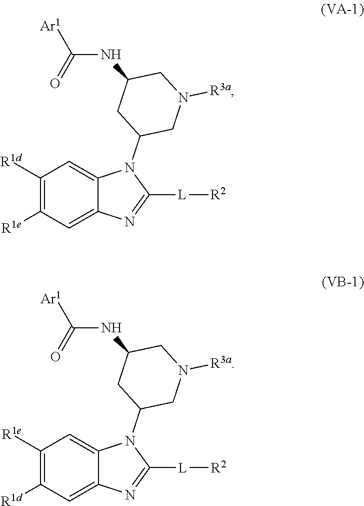

Aspect 205. The compound according to aspect 202, or a pharmaceutically acceptable salt thereof, wherein the compound is a compound of Formula VC or Formula VD.
Aspect 206. The compound according to aspect 205, wherein the compound of Formula VC is a compound of Formula VC-1, or a pharmaceutically acceptable salt thereof, and the compound of Formula VD is a compound of Formula VD-1, or a pharmaceutically acceptable salt thereof:

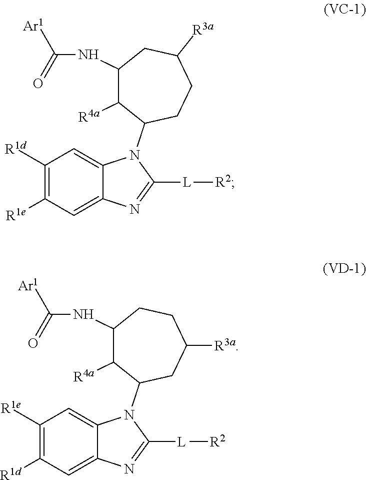

Aspect 207. The compound according to any one of aspects 202-206, or a pharmaceutically acceptable salt thereof, wherein one of R^{3a} or R^{4a} is OH or C₁-C₃alkoxy; and the other of R^{3a} or R^{4a} is hydrogen.
Aspect 208. The compound according to any one of aspects 202-207, or a pharmaceutically acceptable salt thereof, wherein L is a bond and R² is optionally substituted phenyl or optionally substituted pyridinyl.
Aspect 209. The compound according to any one of aspects 202-208, or a pharmaceutically acceptable salt thereof, wherein Ar¹ is optionally substituted thiophenyl or optionally substituted thiazolyl.
Aspect 210. The compound according to any one of aspects 202-209, or a pharmaceutically acceptable salt thereof, wherein R^{1d} is CONH(CH₃) and R^{1e} is hydrogen.
Aspect 211. The compound of aspect 83 that is a compound of Table 1, or a pharmaceutically acceptable salt thereof.
Aspect 212. The compound of aspect 178 that is a compound of Table 2, or a pharmaceutically acceptable salt thereof.
Aspect 213. The compound of aspect 185 that is a compound of Table 3, or a pharmaceutically acceptable salt thereof.
Aspect 214. The compound of aspect 194 that is a compound of Table 4, or a pharmaceutically acceptable salt thereof.
Aspect 215. The compound of aspect 202 that is a compound of Table 5, or a pharmaceutically acceptable salt thereof.
Aspect 216. A pharmaceutical composition comprising a compound according to any one of aspects 83-215, or a pharmaceutically acceptable salt thereof, and a pharmaceutically acceptable excipient.
Aspect 217. A method of reducing plasma methylmalonic acid in a subject having an elevated methylmalonic acid level comprising administering to the subject a therapeutically effective amount of a compound of any one of aspects 83-215.
Aspect 218. A method of treating a BCAA-catabolism-related diseases in a subject in need thereof comprising administering to the subject a therapeutically effective amount of a compound, or a pharmaceutically acceptable salt thereof, of any one of aspects 83-215.
Aspect 219. The method according to aspect 218, wherein the BCAA-catabolism-related disease is methylmalonic acidemia (MMA), propionic acidemia (PA), isovaleric acidemia (IVA), maple syrup urine disease, methylmalonic semialdehyde (MMSDH) deficiency, 3-hydroxyisobutyrate dehydrogenase (HIBADH) deficiency, 3-hydroxyisobutyryl-CoA hydrolase (HIBCH) deficiency, isobutyryl-CoA (IBD) deficiency, acetyl-CoA carboxylase 1 (ACC) deficiency, mitochondrial short-chain enoyl-CoA hydratase 1 (ECHS1) deficiency, methylbutyryl CoA dehydrogenase (SBCAD) deficiency, beta-ketothiolase deficiency (BKT), hydroxy-3-methylglutaconyl-CoA lysase (HMGCL) deficiency, 3-methylglutaconyl-CoA-hydratase deficiency, or 3-methylcrotonyl-CoA (3-MCC) deficiency.

### EXAMPLES

The present disclosure will be more fully understood by reference to the following examples. The examples should not, however, be construed as limiting the scope of the present disclosure

### Units and Terms List

anhy. anhydrous
aq. aqueous
atm atmosphere
min minute(s)
hrs hours
mL milliliter
μL microliter
mmol millimole(s)
μmol micromole(s)
mol mole(s)
MS mass spectrometry
LC-MS Liquid chromatography-mass spectrometry
NMR nuclear magnetic resonance
TLC thin layer chromatography
HPLC high-performance liquid chromatography
sat. saturated
° C. degrees Celsius
rt room temperature
wt weight
N₂ nitrogen gas
H₂ hydrogen gas

NMR Spectra
Hz hertz
δ chemical shift
s singlet
d doublet
t triplet
q quartet
m multiplet
br broad
dd doublet of doublets
CDCl₃ chloroform-d
CD₃OD methanol-d₄
DMSO-d₆ dimethylsulfoxide-d₆
CD₃CN acetonitrile-d₃

Solvents and Reagents:
CHCl₃ chloroform
DCM dichloromethane
DCE 1,2-dichloroethane
DMF dimethylformamide
EtOH ethyl alcohol
EtOAc ethyl acetate
MeOH methyl alcohol
MeCN or ACN acetonitrile
PE petroleum ether
THF tetrahydrofuran
DMSO dimethyl sulfoxide
t-BuOH tert-butanol
AcOH acetic acid
HCl hydrochloric acid
FA formic acid
NH₄Cl ammonium chloride
NaOH sodium hydroxide
LiOH lithium hydroxide
K₂CO₃ potassium carbonate
Na₂CO₃ sodium carbonate
Cs₂CO₃ cesium carbonate
TFA trifluoroacetic acid
Na₂SO₄ sodium sulfate
Na₂CO₃ sodium carbonate
NaHCO₃sodium bicarbonate
K₃PO₄ potassium phosphate
NaH sodium hydride
MeONa sodium methoxide
MeMgBr methylmagnesium bromide
CH₃I or Mel methyl iodide
NH₄OH ammonium hydroxide
Et₃N or TEA triethylamine
DIEA N,N-diisopropylethylamine
DIPA diisopropylamine
DMEDA N,N′-Dimethylethylenediamine
Py pyridine
EDCI 1-ethyl-3-(3-dimethylaminopropyl)carbodiimide
HOBt 1-hydroxybenzotriazole
HATU 1-[Bis(dimethylamino)methylene]-1H-1,2,3-triazolo[4,5-b]pyridinium 3-oxid hexafluorophosphate
CDI carbonyldiimidazole
DBU 1,8-diazabicyclo[5.4.0]undec-7-ene
Boc₂O di-tert-butyl dicarbonate
H₂O water
K₂OsO₄·2H₂O potassium osmate(VI) dihydrate
Fe iron
Pd/C palladium on carbon
Pd(PPh₃)₄ tetrakis(triphenylphosphine)palladium(0)
Pd(PPh₃)₂Cl₂ bis(triphenylphosphine)palladium(II) dichloride
Pd(OAc)₂ palladium (II) acetate
Pd(dppf)Cl₂ (1,1′-Bis(diphenylphosphino)ferrocene)palladium(II) dichloride
t-BuXphos-Pd-G3 [(2-Di-tert-butylphosphino-2′,4′,6′-triisopropyl-1,1′-biphenyl)-2-(2′-amino-1,1′-biphenyl)]palladium(II) methanesulfonate
Cu(OAc)₂ copper (II) acetate
CuI copper iodine
CuCl copper chloride
NaN₃ sodium azide
SOCl₂ thionyl chloride
NaI sodium iodide
Zn(CN)₂ zinc cyanide
TBDPSCI tert-butyldiphenylsilyl chloride
TsCl p-toluenesulfonyl chloride
DEAD diethyl azodicarboxylate
TBAF tetra-n-butylammonium fluoride
TMSN₃ trimethylsilyl azide

### General Experimental

In the following examples, the reagents and solvents were purchased from commercial sources (such as Alfa, Acros, AstaTech, CombiBlocks, Enamine, Sigma Aldrich, TCI, PharmaBock, Bide Pharmatech Ltd., Accela ChemBio, Aladdin, Shanghai Haohong Pharmaceutical Co., Ltd, Amkchem, Beijing Ouhe Technology Co., Ltd, Haoyuan Chemexpress Co., Ltd, Hualun, Coolpharm, Scochem, Titan and WuXi LabNetwork), and used without further purification unless otherwise specified. Flash chromatography was performed on a CombiFlashRf 150 (ISCO), CombiFlash Rf⁺, or CombiFlash Rf⁺ Lumen via columns with silica gel particles of 200-300 mesh. HPLC was performed on an Agilent 1100 Liquid Chromatography (Agilent, USA), a Shimadzu LC 20/20A (Shimadzu, Japan), and a Waters Acquity UPLC (Waters, USA). Supercritical fluid chromatography was performed on a Waters Prep SFC 150 AP/80Q/200/350 system (Waters, USA). Analytical and preparative thin layer chromatography plates (TLC) were HSGF 254 (0.15-0.2 mm thickness, Shanghai Anbang Company, China). Nuclear magnetic resonance (NMR) spectra were obtained on a Brucker AV-400 NMR (Bruker, Switzerland). Chemical shifts were reported in parts per million (ppm, 6) downfield from tetramethylsilane. Mass spectra were given with electrospray ionization (ESI) from a Waters LCT TOF Mass Spectrometer (Waters, USA). LC-MS was performed on an Agilent Prime-6125B/Agilent LC1260-MS6150/Agilent LC1260-MS6125B/Agilent LC1200-MS6110 (Agilent, USA), a Shimadzu LC20-MS2020, and a Waters Acquity UPLC-H Class. Microwave reactions were run on an Initiator 2.5 Microwave Synthesizer (Biotage, Sweden). Lyophilizations were done on EYELA Freeze Dryer FDU-2100 (TOKYO RIKAKIKAI, Japan) or FreeZone 6 Liter Freeze Dryer (LABCONCO, USA).

Preparation of intermediates of formula 1.2 and formula 1.3:

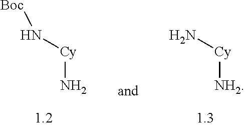

The following Intermediate Schemes 1 to 13 describe the syntheses of intermediates of formula 1.2 or formula 1.3. Additional intermediates of formulas 1.2 and 1.3 are commercially available such as, for example, tert-butyl ((1S,3R)-3-aminocyclohexyl)carbamate, tert-butyl (3-aminocyclohexyl)carbamate, and tert-butyl (5-aminotetrahydro-2H-pyran-3-yl)carbamate.

Intermediate Scheme 1. Preparation of intermediate tert-butyl (3-amino-2-hydroxycyclohexyl)carbamate (relative stereochemistry is indicated in Intermediate Scheme 1)

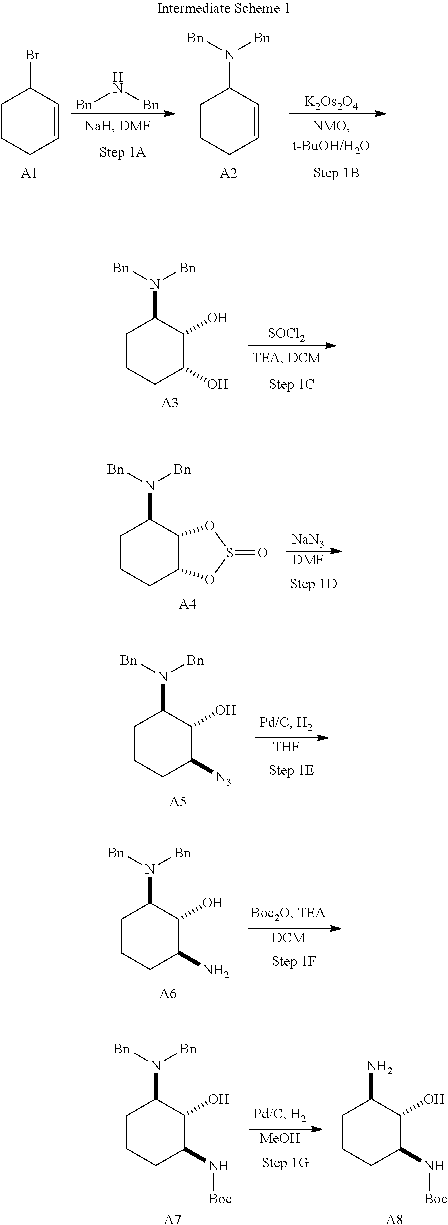

### Step 1A. Synthesis of N,N-dibenzylcyclohex-2-en-1-amine (A2)

A mixture of N-benzyl-1-phenyl-methanamine (3.0 g, 15.2 mmol, 2.91 mL) and 3-bromocyclohexene (A1) (2.45 g, 15.2 mmol, 1.75 mL) in DMF (12 mL) was degassed and purged with N₂ gas 3 times, and then NaH (639 mg, 16.0 mmol, 60% purity) was added to the reaction mixture at 0° C. The reaction mixture was stirred at rt for 12 hrs under N₂ atmosphere, then quenched by the addition of H₂O (15 mL) at 0° C., followed by the addition of additional H₂O (200 mL), and the aqueous portion extracted with EtOAc (200 mL×3). The combined organic layers were washed with brine (300 mL×3), dried over Na₂SO₄, filtered and concentrated to dryness. The residue was purified by silica gel column chromatography to give A2 (5.3 g, 62.8% yield). LC-MS: m/z 278.2 [M+H]⁺.

### Step 1B. Synthesis of 3-(dibenzylamino)cyclohexane-1,2-diol (A3)

To a mixture of N,N-dibenzylcyclohex-2-en-1-amine (A2) (1.93 g, 6.96 mmol) in t-BuOH (40 mL) and H₂O (8 mL) were added K₂OsO₄·2H₂O (28.2 mg, 76.5 μmol) and 4-methyl-4-oxido-morpholin-4-ium (897 mg, 7.65 mmol). The reaction mixture was stirred at rt for 14 hrs. The reaction mixture was treated with sat. aq. sodium hydrosulfite solution (5 mL) and stirred for 1 hr, and then concentrated under reduced pressure. The aqueous portion was extracted with DCM (100 mL×3). The combined organic layers were concentrated to dryness, and the residue was purified by silica gel column chromatography to give A3 (1.63 g, 75.2% yield). LC-MS: m/z 312.2 [M+H]⁺.

### Step 1C. Synthesis of 4-(dibenzylamino)hexahydrobenzo[d][1,3,2]dioxathiole 2-oxide (A4)

To a solution of 3-(dibenzylamino)cyclohexane-1,2-diol (A3) (1.63 g, 5.23 mmol) in DCM (10 mL) was added Et₃N (4.24 g, 41.9 mmol). SOCl₂ (1.87 g, 15.7 mmol) was then added slowly to the reaction mixture at 0° C. The reaction mixture was stirred at 0° C. to rt for 2 hrs, then poured into H₂O (80 mL) at 5° C. Additional water (20 mL) was added, and the aqueous portion extracted with DCM (80 mL×3). The combined organic layers were washed with brine (80 mL×3), dried over Na₂SO₄, filtered, and concentrated to dryness. The residue was purified by silica gel column chromatography to give A4 (940 mg, 50.2% yield). LC-MS: m/z 358.2 [M+H]⁺.

### Step 1D. Synthesis of 2-azido-6-(dibenzylamino)cyclohexan-1-ol (A5)

To a solution of 4-(dibenzylamino)hexahydrobenzo[d][1,3,2]dioxathiole 2-oxide (A4) (100 mg, 280 μmol) in DMF (2 mL) was added NaN₃ (54.6 mg, 839 μmol) at rt. The reaction mixture was heated at 100° C. for 30 hrs. The reaction mixture was cooled to rt, quenched by the addition of brine (10 mL) at 15° C., then H₂O (10 mL) was added, and the aqueous portion extracted with DCM (20 mL×3). The combined organic layers were washed with brine (20 mL×3), dried over Na₂SO₄, filtered, and concentrated to dryness. The residue was purified by silica gel column chromatography to give A5 (55 mg, 58.4% yield). LC-MS: m/z 337.2 [M+H]⁺.

### Step 1E. Synthesis of 2-amino-6-(dibenzylamino)cyclohexan-1-ol (A6)

To a solution of 2-azido-6-(dibenzylamino)cyclohexan-1-ol (A5) (342 mg, 1.02 mmol) in THF (1 mL) was added Pd/C (50 mg, 1.02 mmol, 10% purity) under N₂ atmosphere. The suspension was degassed and purged with H₂ 3 times. The reaction mixture was stirred under H₂ (15 psi) at rt for 1.2 hrs. The reaction mixture was filtered, and the filter cake washed with THF (8 mL×3). The combined organic layers were concentrated to dryness to give A6 (321 mg), which was used without further purification. LC-MS: m/z 311.2 [M+H]⁺.

### Step 1F. Synthesis of tert-butyl (3-(dibenzylamino)-2-hydroxycyclohexyl)carbamate (A7)

To a solution of 2-amino-6-(dibenzylamino)cyclohexan-1-ol (A6) (321 mg, 1.03 mmol) in DCM (4 mL) were added Et₃N (314 mg, 3.10 mmol) and tert-butoxycarbonyl tert-butyl carbonate (237 mg, 1.09 mmol). The reaction mixture was stirred at rt for 13 hrs. The reaction mixture was quenched by the addition of H₂O (5 mL) at 15° C., diluted with H₂O (15 mL) and the aqueous portion extracted with DCM (20 mL×3). The combined organic layers were washed with brine (20 mL×3), dried over Na₂SO₄, filtered, and concentrated to dryness. The residue was purified by silica gel column chromatography to give A7 (350 mg, 82.5% yield). LC-MS: m/z 411.3 [M+H]⁺.

### Step 1G. Synthesis of tert-butyl (3-amino-2-hydroxycyclohexyl)carbamate (A8)

To a solution of tert-butyl (3-(dibenzylamino)-2-hydroxycyclohexyl)carbamate (A7) (350 mg, 853 μmol) in MeOH (5 mL) was added Pd/C (100 mg, 10% Pd) under N₂ atmosphere. The suspension was degassed and purged with H₂ 3 times. The reaction mixture was stirred under H₂ (15 Psi) at rt for 3 hrs. The reaction mixture was filtered, and the filter cake was washed with MeOH (10 mL×3). The combined organic layers were concentrated to dryness to give A8 (202 mg), which was used without further purification. ¹H NMR (400 MHz, CDCl₃) δ 5.11 (s, 1H), 3.40 (s, 1H), 3.24 (s, 1H), 2.87 (s, 1H), 1.98 (br d, 2H), 1.71 (br dd, 1H), 1.44 (s, 9H), 1.38-1.20 (m, 3H).

Intermediate Scheme 2: Preparation of intermediate 5-methoxycyclohexane-1,3-diamine (relative stereochemistry is indicated in Intermediate Scheme 2)

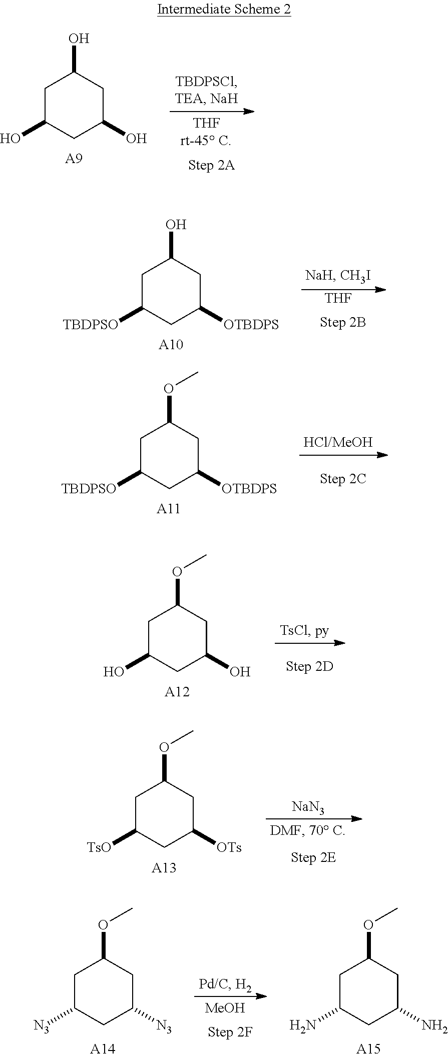

### Step 2A. Synthesis of 3,5-bis((tert-butyldiphenylsilyl)oxy)cyclohexan-1-ol (A10)

To a solution of cis, cis-cyclohexane-1,3,5-triol (A9) (5 g, 37.8 mmol) in THF (100 mL) was added TBDPSCl (21.84 g, 79.5 mmol, 20.4 mL) and TEA (11.49 g, 114 mmol, 15.8 mL) at rt. After addition, the reaction mixture was stirred at rt for 1.5 hrs and treated with NaH (4.54 g, 114 mmol, 60% purity). The reaction mixture was heated to 45° C. for 22.5 hrs under N₂ atmosphere. The reaction mixture was concentrated under reduced pressure. Sat. aq. NH₄Cl solution (50 mL) was added to the reaction mixture, and the aqueous portion was extracted with EtOAc (100 mL×3). The combined organic layers were washed with brine (20 ml), dried over Na₂SO₄, filtered, and concentrated to dryness. The residue was purified by flash silica gel chromatography to give A10 (8.7 g, 35.0% yield). LC-MS: m/z 609.2 (M+H)⁺

### Step 2B. Synthesis of ((5-methoxycyclohexane-1,3-diyl)bis(oxy))bis(tert-butyldiphenylsilane) (A11)

To a solution of 3,5-bis((tert-butyldiphenylsilyl)oxy)cyclohexan-1-ol (A10) (1.0 g, 1.64 mmol) in THF (12 mL) was added NaH (98.5 mg, 2.46 mmol, 60% purity). The reaction mixture was stirred at rt for 30 min and treated with CH₃I (280 mg, 1.97 mmol, 123 μL). The reaction mixture was stirred at rt for 16 hrs under N₂ atmosphere. The reaction mixture was concentrated under reduced pressure. Sat. aq. NH₄Cl solution (15 mL) was added, and the aqueous portion extracted with EtOAc (30 mL×3). The combined organic layers were washed with brine (20 ml), dried over Na₂SO₄, filtered, and concentrated to dryness. The residue was purified by flash silica gel chromatography to give A11 (1.0 g, 96.8% yield). LC-MS: m/z 623.3 (M+H)⁺.

### Step 2C. Synthesis of 5-methoxycyclohexane-1,3-diol (A12)

To a solution of ((5-methoxycyclohexane-1,3-diyl)bis(oxy))bis(tert-butyldiphenylsilane) (A11) (700 mg, 1.12 mmol) in MeOH (7 mL) was added HCl/MeOH (4 M, 7 mL). The reaction mixture was stirred at rt for 15 hrs and concentrated to dryness to afford A12 (170 mg). LC-MS: m/z 147.1 (M+H)⁺.

### Step 2D. Synthesis of 5-methoxycyclohexane-1,3-diyl bis(4-methylbenzenesulfonate) (A13)

To a solution of 5-methoxycyclohexane-1,3-diol (A12) (170 mg, 1.16 mmol) in pyridine (7 mL) was added TsCl (887 mg, 4.65 mmol). The mixture was stirred at rt for 63 hrs. The reaction mixture was concentrated to dryness. The residue was purified by flash silica gel chromatography to give A13 (440 mg, 67.4% yield). LC-MS: m/z 477.1 (M+Na)⁺.

### Step 2E. Synthesis of 1,3-diazido-5-methoxycyclohexane (A14)

To a solution of 5-methoxycyclohexane-1,3-diyl bis(4-methylbenzenesulfonate) (A13) (440 mg, 968 μmol) in DMF (5 mL) was added NaN₃ (378 mg, 5.81 mmol). The reaction mixture was stirred at 70° C. for 15 hrs. The reaction mixture was cooled to rt, and EtOAc (100 mL) was added. The organic layer was washed with H₂O (40 mL×5), brine (30 mL), dried over Na₂SO₄, filtered, and concentrated to dryness to afford A14 (180 mg). ¹H NMR (400 MHz, CDCl₃) δ 3.75-3.61 (m, 3H), 3.32 (s, 3H), 2.35-2.21 (m, 3H), 1.41-1.30 (m, 3H).

### Step 2F. Synthesis of 5-methoxycyclohexane-1,3-diamine (A15)

To a solution of 1,3-diazido-5-methoxycyclohexane (A14) (180 mg, 917 μmol) in MeOH (2 mL) was added Pd/C (30 mg, 917 μmol, 10% purity) under N₂ atmosphere. The reaction mixture was degassed and purged with H₂ for 3 times and stirred under H₂ (15 Psi) at rt for 15 hrs. The reaction mixture was filtered through Celite, and the filtrate concentrated to dryness to afford 5-methoxycyclohexane-1,3-diamine (90 mg). ¹H NMR (400 MHz, CDCl₃) δ 3.61-3.56 (m, 1H), 3.25 (s, 3H), 2.99-2.93 (m, 2H), 2.02-1.94 (m, 3H), 1.04-0.97 (m, 2H), 0.88-0.79 (m, 1H).

Alternatively, 5-methoxycyclohexane-1,3-diamine (A15) may be prepared in accordance with Intermediate Scheme 2A (relative stereochemistry is indicated in Intermediate Scheme 2A). Steps 2E and 2F were performed following the procedures set forth in the Electronic Supplementary Materials (Scheme S1) for Dalton Trans., 2019, 48, 9576-9580. Steps 2G and 2H were performed following the procedures set forth in New J. Chem., 2005, 29, 1152-1158 (Scheme 2).

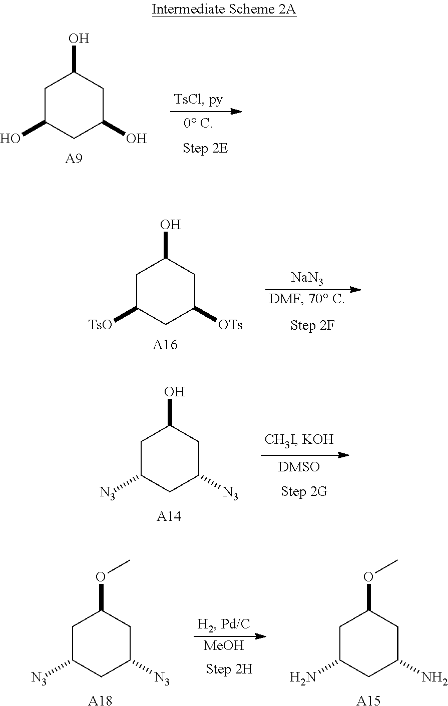

Intermediate Scheme 3: Preparation of intermediate tert-butyl-3-amino-2-methoxycyclohexylcarbamate (relative stereochemistry is indicated in Intermediate Scheme 3)

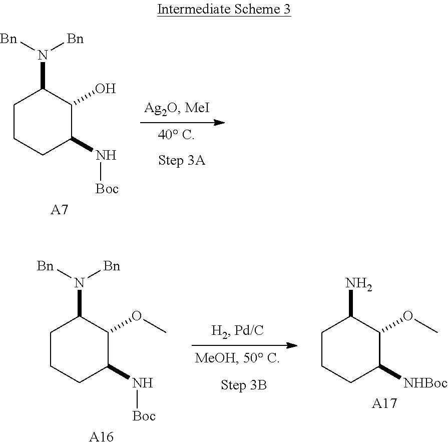

### Step 3A. Synthesis of tert-butyl-(3-(dibenzylamino)-2-methoxycyclohexyl)carbamate (A16)

To a solution of tert-butyl (3-(dibenzylamino)-2-hydroxycyclohexyl)carbamate (A7) (1.70 g, 4.14 mmol) in CH₃I (20 mL) was added silver oxide (2.88 g, 12.4 mmol). The reaction mixture was then degassed under vacuum and purged with N₂ several times, and heated at 40° C. for 48 hrs under N₂ atmosphere in a sealed tube. The reaction mixture was filtered through a pad of Celite and the filter cake was washed with EtOAc (20 mL). The filtrate was concentrated to dryness to afford A16 (1.72 g), which was used without further purification. LC-MS: m/z 425 [M+H]⁺.

### Step 3B. Synthesis of tert-butyl-3-amino-2-methoxycyclohexylcarbamate (A17)

To a solution of tert-butyl-(3-(dibenzylamino)-2-methoxycyclohexyl)carbamate (A16) (1.72 g, 4.05 mmol) in MeOH (10 mL) was added Pd/C (431 mg, 10% wt) under N₂ atmosphere. The reaction mixture was degassed and purged with H₂ several times, then heated at 50° C. for 18 hrs under H₂ atmosphere. The reaction mixture was filtered through a pad of Celite, and the filter cake was washed with MeOH (20 mL). The filtrate was concentrated to dryness to afford A17 (857 mg), which was used without further purification. LC-MS: m/z 245 [M+H]⁺.

Intermediate Scheme 4: Preparation of intermediate rel-tert-butyl ((1R,3S)-3-amino-2-hydroxy-2-methylcyclohexyl)carbamate (relative stereochemistry is indicated in Intermediate Scheme 4 for steps 4A and 4B)

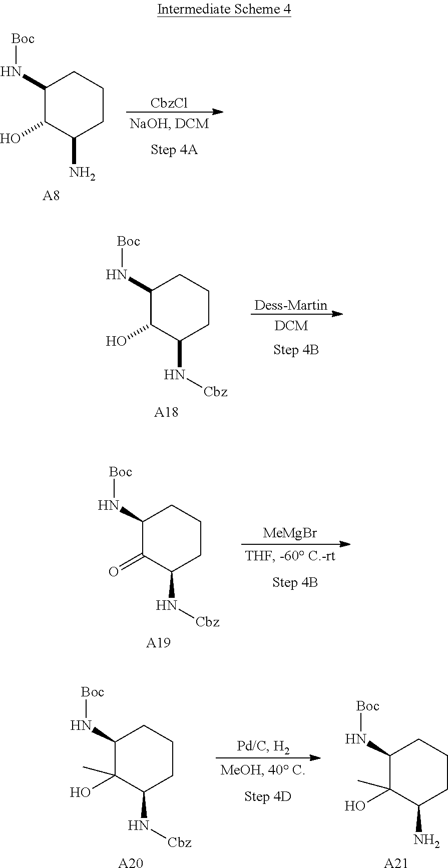

### Step 4A. Synthesis of benzyl tert-butyl (2-hydroxycyclohexane-1,3-diyl)dicarbamate (A18)

To a solution of tert-butyl (3-amino-2-hydroxycyclohexyl)carbamate (A8) (60 mg, 261 μmol) in DCM (3 mL) was added benzyl carbonochloridate (53.3 mg, 313 μmol, 44.4 μL), and then aq. NaOH solution (52.1 mg, 521 μmol, 1 M) was added. The reaction mixture was stirred at rt for 3 hrs and concentrated to dryness. EtOAc (10 mL) was added to the residue, and it was stirred for 1 hr. The organic layer was washed with brine (5 mL×3), dried over Na₂SO₄, filtered, and concentrated to dryness to afford A18 (90 mg), which was used without further purification. LC-MS: m/z 365.2 [M+H]⁺.

### Step 4B. Synthesis of benzyl tert-butyl (2-oxocyclohexane-1,3-diyl) dicarbamate (A19)

To a solution of tert-butyl (2-hydroxycyclohexane-1,3-diyl)dicarbamate (A18) (90 mg, 247 μmol) in DCM (2 mL) was added Dess-Martin reagent (115 mg, 272 μmol). The reaction mixture was stirred at rt for 3 hrs. The reaction mixture was quenched by the addition of sat. aq. NaHCO₃ solution (5 mL), and then H₂O (5 mL) was added. The aqueous portion was extracted with EtOAc (10 mL×3). The combined organic layers were washed with brine (10 mL×3), dried over Na₂SO₄, filtered, and concentrated to dryness. The residue was purified by column chromatography to give A19 (73 mg, 81.6% yield). LC-MS: m/z 363.2 [M+H]⁺.

### Step 4C. Synthesis of rel-benzyl tert-butyl ((1R,3 S)-2-hydroxy-2-methylcyclohexane-1,3-diyl)dicarbamate (A20)

A mixture of benzyl tert-butyl (2-oxocyclohexane-1,3-diyl)dicarbamate (A19) (73 mg, 201 μmol) in THF (2 mL) was degassed and purged with N₂ for 3 times, and then MeMgBr (3 M, 235 μL) was added to the reaction mixture. The reaction mixture was then stirred at −60° C. for 2 hrs under N₂ atmosphere, and then stirred at rt for 1 hr under N₂ atmosphere. The reaction mixture was quenched by the addition of sat. aq. NH₄Cl solution (5 mL) at 10° C. Water (5 mL) was added, and the aqueous portion was extracted with EtOAc (10 mL×3). The combined organic layers were washed with brine (10 mL×3), dried over Na₂SO₄, filtered, and concentrated to dryness. The residue was purified by column chromatography to give A20 (40 mg, 52.5% yield). LC-MS: m/z 401.2 [M+Na]⁺.

### Step 4D. Synthesis of rel-tert-butyl ((1S,3R)-3-amino-2-hydroxy-2-methylcyclohexyl)-carbamate (A21)

A mixture of rel-benzyl tert-butyl ((1R,3S)-2-hydroxy-2-methylcyclohexane-1,3-diyl)dicarbamate (A20) (590 mg, 1.56 mmol) in MeOH (3 mL) was degassed and purged with N₂ for 3 times, and then Pd/C (400 mg, 10% purity) was added to the reaction mixture. The reaction mixture was degassed and purged with H₂ 3 times and stirred at 40° C. for 12 hrs under H₂ atmosphere (15 Psi). The reaction mixture was filtered through a Celite pad, and the filter cake washed with MeOH (5 mL×3), The combined organic layers were concentrated to dryness to afford A21 (368 mg), which was used without further purification. The obtained product was one stereoisomer. The stereochemistry of the carbon with the methyl and hydroxy substituents has not been assigned.

### Intermediate Scheme 5:

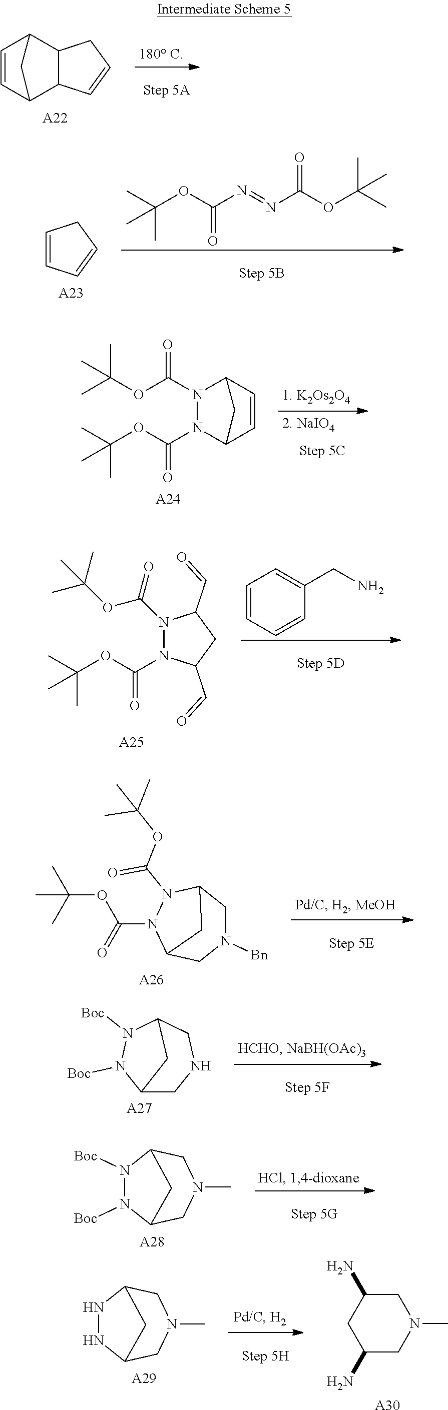

### Step 5A. Synthesis of cyclopenta-1,3-diene (A23)

Tricyclodeca-2,4-diene (A22) (50 g, 378 mmol) was distilled at 180° C. for 5 hrs to give A23 (38.1 g, 76.2% yield). ¹H NMR (400 MHz, CDCl₃) δ 6.58 (d, 2H), 6.47 (t, 2H), 2.98 (s, 2H).

### Step 5B. Synthesis of di-tert-butyl 2,3-diazabicyclo[2.2.1]hept-5-ene-2,3-dicarboxylate (A24)

A solution of di-tert-butyl (E)-diazene-1,2-dicarboxylate (5 g, 21.7 mmol) in DCM (50 mL) was cooled to 0° C. Cyclopenta-1,3-diene (A23) (7.18 g, 109 mmol) was then added. The reaction mixture was stirred at 0° C. for 4 hrs, and then allowed to warm to rt for 12 hrs. The reaction mixture was concentrated to dryness. The residue was purified by column chromatography on silica gel to give A24 (6.3 g, 97.9% yield). LC-MS: m/z 319.3 [M+Na]⁺.

### Step 5C. Synthesis of di-tert-butyl 3,5-diformylpyrazolidine-1,2-dicarboxylate (A25)

To a solution of di-tert-butyl 2,3-diazabicyclo[2.2.1]hept-5-ene-2,3-dicarboxylate (A24) (1.0 g, 3.37 mmol) and K₂OsO₄·2H₂O (124 mg, 337 μmol) in THF (30 mL) was added a solution of NaIO₄ (3.61 g, 16.9 mmol, 935 μL) in H₂O (15 mL). After the addition was complete, the reaction mixture was stirred at rt for 16 hrs. Aq. sat. Na₂SO₃ solution (50 mL) was added, and the aqueous portion extracted with EtOAc (3×30 mL). The combined organic layers were washed with brine (35 mL), dried over Na₂SO₄, filtered, and concentrated to dryness. The residue was purified by column chromatography on silica gel to give A25 (0.57 g, 51.5% yield). ¹H NMR (400 MHz, CDCl₃) δ 9.65 (s, 2H), 4.72 (t, 2H), 2.61 (t, 2H), 1.50 (s, 18H).

### Step 5D. Synthesis of di-tert-butyl 3-benzyl-3,6,7-triazabicyclo[3.2.1]octane-6,7-dicarboxylate (A26)

A solution of di-tert-butyl 3,5-diformylpyrazolidine-1,2-dicarboxylate (A25) (570 mg, 1.74 mmol), phenylmethanamine (186 mg, 1.74 mmol, 189 μL) and one drop of AcOH (10 μL) in DCE (5 mL) was stirred at rt for 1 hr. Then NaBH(OAc)₃ (1.47 g, 6.94 mmol) was added, and the reaction mixture was stirred at rt for another 16 hrs. Water (20 mL) was added, and the aqueous portion was extracted with DCM (3×20 mL). The combined organic layers were washed with brine (30 mL), dried over Na₂SO₄, filtered, and concentrated to dryness. The residue was purified by column chromatography on silica gel to give A26 (0.3 g, 39.9% yield). ¹H NMR (400 MHz, CDCl₃) δ 7.32-7.25 (m, 5H), 4.46-4.32 (m, 3H), 3.68-3.50 (m, 3H), 3.25-3.09 (m, 2H), 2.14-1.91 (m, 2H) 1.51 (s, 18H); LC-MS: m/z 404.3 [M+H]⁺.

### Step 5E. Synthesis of di-tert-butyl 3,6,7-triazabicyclo[3.2.1]octane-6,7-dicarboxylate (A27)

To a solution of di-tert-butyl 3-benzyl-3,6,7-triazabicyclo[3.2.1]octane-6,7-dicarboxylate (A26) (100 mg, 248 μmol) in MeOH (2 mL) was added Pd/C (0.02 g, 10% Pd) under N₂. The suspension was degassed under vacuum and purged with H₂ several times. The reaction mixture was stirred under H₂ (15 psi) at rt for 16 hrs, and then filtered. The filtrate was concentrated to dryness to give A27 (80 mg). LC-MS: m/z 314.3 [M+H]⁺.

### Step 5F. Synthesis of di-tert-butyl 3-methyl-3,6,7-triazabicyclo[3.2.1]octane-6,7-dicarboxylate (A28)

To a solution of di-tert-butyl 3,6,7-triazabicyclo[3.2.1]octane-6,7-dicarboxylate (A27) (0.2 g, 638 μmol) and one drop of AcOH (10 μL) in DCE (5 mL) was added formaldehyde (77.7 mg, 957 μmol, 71.3 μL) (37% in H₂O). After the addition was complete, the reaction mixture was stirred at rt for 1 hr. Then NaBH(OAc)₃ (271 mg, 1.28 mmol) was added, and the reaction mixture was stirred at rt for another 16 hrs. The reaction mixture was concentrated to dryness, and the residue was purified by column chromatography on silica gel to give A28 (0.2 g, 87.7% yield). ¹H NMR (400 MHz, CD₃OD) δ 4.40-4.15 (m, 2H), 3.20-2.95 (m, 2H), 2.23 (s, 3H), 2.17-1.80 (m, 4H), 1.47 (s, 18H). LC-MS: m/z 328.2 [M+H]⁺.

### Step 5G. Synthesis of 3-methyl-3,6,7-triazabicyclo[3.2.1]octane (A29)

A solution of di-tert-butyl 3-methyl-3,6,7-triazabicyclo[3.2.1]octane-6,7-dicarboxylate (A28) (0.2 g, 611 μmol) in HCl (4 M, 6.78 mL) (4M in 1,4-dioxane) was stirred at rt for 1 hr. The reaction mixture was concentrated to dryness to give A29 (0.15 g).

### Step 5H. Synthesis of 1-methylpiperidine-3,5-diamine (A30) (relative stereochemistry at centers 3 and 5 of piperidine ring is cis)

To a solution of 3-methyl-3,6,7-triazabicyclo[3.2.1]octane (A29) (150 mg, 750 μmol, 2HCl) in MeOH (2 mL) was added Pd/C (0.01 g, 10% Pd) under N₂. The suspension was degassed under vacuum and purged with H₂ several times. The mixture was stirred under H₂ (50 psi) at 50° C. for 16 hrs. The reaction mixture was filtered, and the filtrate was concentrated to dryness to give A30 (0.14 g, 92.4% yield, 2HCl salt). ¹H NMR (400 MHz, CD₃OD) δ 8.46 (m, 5H), 3.17-3.16 (m, 2H), 2.99 (d, 2H), 2.39-2.27 (m, 1H), 2.26 (s, 3H), 1.90 (t, 2H), 1.45-1.42 (m, 1H); LC-MS: m/z 130.3 [M+H]⁺.

### Synthesis of 1-(3,5-diaminopiperidin-1-yl)ethan-1-one (A33)

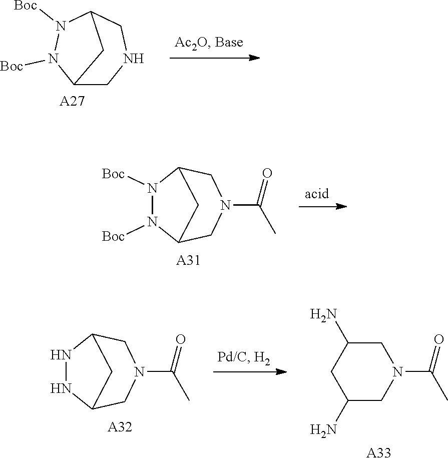

1-(3,5-diaminopiperidin-1-yl)ethan-1-one (A33) (relative stereochemistry at centers 3 and 5 of piperidine ring is cis) may be prepared in accordance with the above scheme by first reacting A27 with acetic anhydride and a base such as TEA to afford di-tert-butyl 3-acetyl-3,6,7-triazabicyclo[3.2.1]octane-6,7-dicarboxylate (A31). The Boc-protecting groups can then be removed by subjecting A31 to acidic conditions such as HCL in 1,4-dioxane to afford 1-(3,6,7-triazabicyclo[3.2.1]octan-3-yl)ethan-1-one (A32). A32 can then be reacted with Pd/C and H₂ gas to afford A33.

### Intermediate Scheme 6: Preparation of rac-tert-butyl (1S,2R,3R)-3-aminocyclohexyl-2-d)carbamate and rac-tert-butyl ((1S,2S,3R)-3-aminocyclohexyl-2-d)carbamate

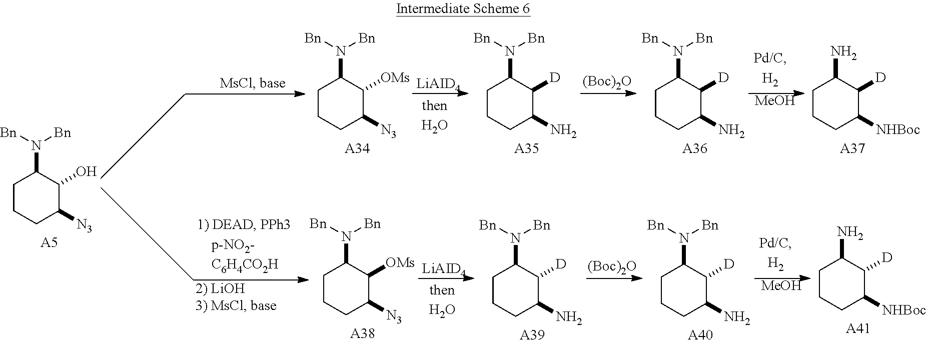

As indicated in Intermediate Scheme 6, rac-tert-butyl ((1S,2R,3R)-3-aminocyclohexyl-2-d)carbamate (A37) may be prepared by first reacting 2-azido-6-(dibenzylamino)cyclohexan-1-ol (A5, racemic) with methanesulfonyl chloride and a suitable base (such as TEA or pyridine) to afford rac-((1S,2S,6R)-2-azido-6-(dibenzylamino)cyclohexyl methanesulfonate (A34). The methanesulfonate group can be subsequently replaced with deuterium through a reduction reaction with lithium aluminum deuteride and water to afford rac-(1R,2S,3S)—N′,N′-dibenzylcyclohexane-2-d-1,3-diamine (A35). The free amine of A35 can be subsequently protected with a tert-butyl carbamate group to afford rac-tert-butyl ((1S,2S,3R)-3-(dibenzylamino)cyclohexyl-2-d)carbamate (A36). The dibenzyl groups of A36 can then be subsequently hydrogenated with Pd/C and H₂ gas in MeOH to afford rac-tert-butyl ((1S,2R,3R)-3-aminocyclohexyl-2-d)carbamate (A37). The enantiomers of A37 may be obtained via SFC separation.

Rac-tert-butyl ((1S,2S,3R)-3-aminocyclohexyl-2-d)carbamate (A41) may be prepared by inverting the stereochemistry of the hydroxyl group of A5 through reaction with DEAD, triphenylphosphine, and para-nitrobenzoic acid, followed by reaction with LiOH and methanesulfonyl chloride and a suitable base (such as TEA or pyridine) to afford rac-((1R,2S,6R)-2-azido-6-(dibenzylamino)cyclohexyl methanesulfonate (A38). The methanesulfonate group of A38 can be subsequently replaced with deuterium through a reduction reaction with lithium aluminum deuteride and water to afford rac-(1R,2R,3S)—N¹,N¹-dibenzylcyclohexane-2-d-1,3-diamine (A39). The free amine of A39 can be subsequently protected with a tert-butyl carbamate protecting group to afford rac-tert-butyl ((1S,2S,3R)-3-(dibenzylamino)cyclohexyl-2-d)carbamate (A40). The dibenzyl groups of A40 can then be subsequently hydrogenated with Pd/C and H₂ gas in MeOH to afford rac-tert-butyl ((1S,2S,3R)-3-aminocyclohexyl-2-d)carbamate (A41). The enantiomers of A41 may be obtained via SFC separation.

Intermediate Scheme 7: Preparation of rac-(1R,3S,5s)-cyclohexane-5-d-1,3-diamine and rac-(1R,3S,5r)-cyclohexane-5-d-1,3-diamine

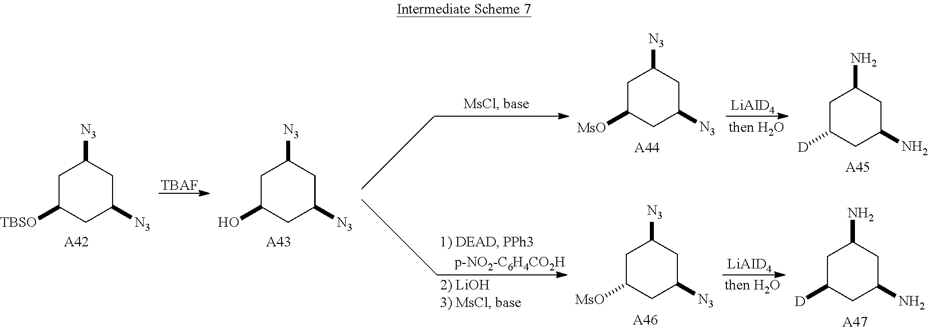

As indicated in Intermediate Scheme 7, rac-(1s,3R,5S)-3,5-diazidocyclohexan-1-ol (A43) may be prepared by removing the tert-butyldimethylsilyl ether protecting group of rac-tert-butyl(((1s,3R,5S)-3,5-diazidocyclohexyl)oxy)dimethylsilane (A42) with TBAF or another suitable desilylation reagent known in the art.

A43 may be subsequently reacted with methanesulfonyl chloride and a suitable base (such as TEA or pyridine) to afford rac-(1s,3R,5S)-3,5-diazidocyclohexyl methanesulfonate (A44). The methanesulfonate group can be subsequently replaced with deuterium through a reduction reaction with lithium aluminum deuteride and water to afford rac-(1R,3S,5s)-cyclohexane-5-d-1,3-diamine (A45). The enantiomers of A45 may be obtained via SFC separation.

Rac-(1R,3S,5r)-cyclohexane-5-d-1,3-diamine (A47) may be prepared by inverting the stereochemistry of the hydroxyl group of (1s,3R,5S)-3,5-diazidocyclohexan-1-ol (A43) through reaction with DEAD, triphenylphosphine, and para-nitrobenzoic acid, followed by reaction with LiOH and methanesulfonyl chloride and a suitable base (such as TEA or pyridine) to afford rac-(1r,3R,5S)-3,5-diazidocyclohexyl methanesulfonate (A46). The methanesulfonate group of A46 can be subsequently replaced with deuterium through a reduction reaction with lithium aluminum deuteride and water to afford rac-(1R,3S,5r)-cyclohexane-5-d-1,3-diamine (A47). The enantiomers of A47 may be obtained via SFC separation.

### Intermediate Scheme 8: Preparation of intermediate 5-methoxycyclohexane-1,3-diamine (relative stereochemistry is indicated in Intermediate Scheme 8)

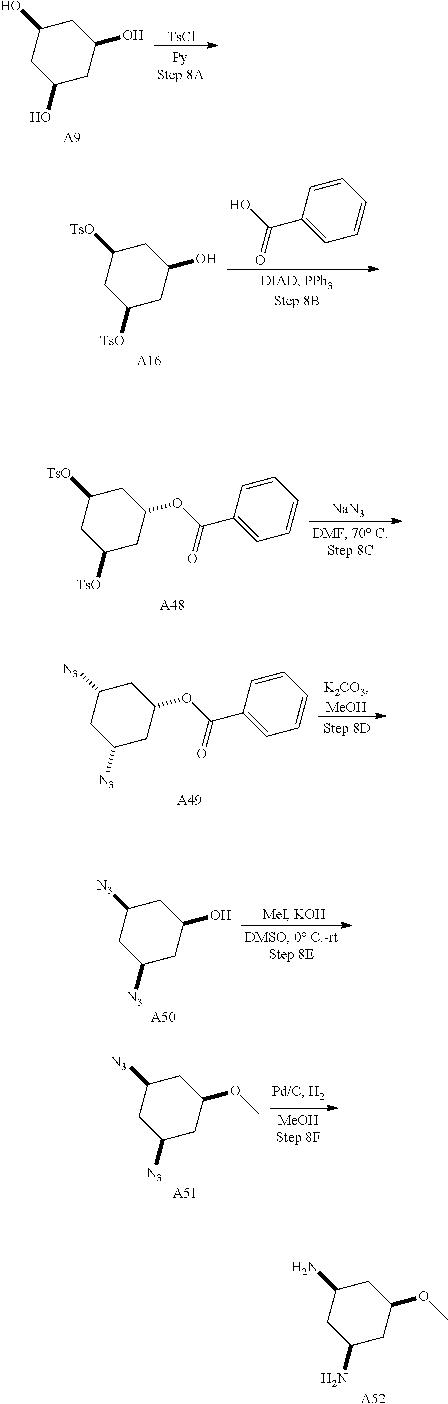

### Step 8A. Synthesis of cis-5-hydroxycyclohexane-1,3-diyl bis(4-methylbenzenesulfonate) (A16)

To a reaction mixture of cis-cyclohexane-1,3,5-triol (16 g, 121 mmol) and TsCl (48.47 g, 254 mmol) was added pyridine (70 mL) at 0° C. The reaction mixture was stirred at 0° C. for 1 hr, and then slowly warmed to rt for 18 hrs. The reaction mixture was diluted with acetone (50 mL), and filtered. The filtrate was concentrated to dryness. Water (80 mL) was added to the residue, and the aqueous portion extracted with EtOAc (50 mL×3). The combined organic layers were washed with 1M HCl (50 mL×2), brine (50 mL), dried over Na₂SO₄, filtered, and concentrated to dryness. The residue was purified by flash silica gel chromatography to give A16 (29.0 g, 54.4% yield). ¹H NMR (400 MHz, CDCl₃) δ 7.77-7.75 (m, 4H), 7.37-7.35 (m, 4H), 4.39-4.32 (m, 2H), 3.59-3.52 (m, 1H), 2.47 (s, 6H), 2.24-2.20 (m, 1H), 1.69-1.62 (m, 2H), 1.49-1.40 (m, 2H). LC-MS: m/z 440.9 [M+H]⁺.

### Step 8B. Synthesis of 3,5-bis(tosyloxy)cyclohexyl benzoate (A48)

To a solution of cis-5-hydroxycyclohexane-1,3-diyl bis(4-methylbenzenesulfonate) (A16) (5.0 g, 9.08 mmol, 80% purity), benzoic acid (1.66 g, 13.6 mmol, 2.08 mL) and PPh₃ (3.57 g, 13.6 mmol) in THF (100 mL) was added DIAD (2.75 g, 13.6 mmol, 2.65 mL) at 0° C., and the reaction mixture was allowed to warm to rt for 18 hrs under N₂. The reaction mixture was concentrated to dryness. The residue was purified by flash silica gel chromatography to give A48 (2.5 g, 50.6% yield). ¹H NMR (400 MHz, CDCl₃) δ 7.90-7.88 (m, 2H), 7.75-7.73 (m, 4H), 7.68-7.66 (m, 1H), 7.54-7.50 (m, 2H), 7.26-7.24 (m, 4H), 5.00-4.97 (m, 1H), 4.76-4.70 (m, 2H), 2.51-2.48 (m, 2H), 2.41 (s, 6H), 2.11-2.07 (m, 2H), 1.81-1.78 (m, 1H), 1.70-1.65 (m, 1H). LC-MS: m/z 567.0 [M+Na]⁺.

### Step 8C. Synthesis of cis-3,5-diazidocyclohexyl benzoate (A49)

To a solution of 3,5-bis(tosyloxy)cyclohexyl benzoate (A48) (3.5 g, 6.43 mmol) in DMF (50 mL) was added NaN₃ (1.88 g, 28.9 mmol), and the reaction mixture was stirred at 70° C. for 18 hrs under N₂. After cooling to rt, the reaction mixture was poured into water (50 mL), and the aqueous portion extracted with EtOAc (30 mL×3). The combined organic layers were washed with brine (20 mL×3), dried over Na₂SO₄, filtered, and most of solvent was evaporated to give a solution of A49 (1.8 g) in EtOAc (24 mL), which was used without further purification.

### Step 8D. Synthesis of cis-3,5-diazidocyclohexan-1-ol (A50)

To a mixture of cis-3,5-diazidocyclohexyl]benzoate (A49) (1.2 g, 4.19 mmol) in EtOAc (16 mL) and K₂CO₃ (1.16 g, 8.38 mmol) was added MeOH (15 mL) and the reaction mixture was stirred at rt for 18 hrs. The reaction mixture was then heated to 50° C. for another 18 hrs. The reaction mixture was concentrated to dryness. The residue was purified by flash silica gel chromatography to give A50 (340 mg), which was used without further purification.

### Step 8E. Synthesis of cis-1,3-diazido-5-methoxycyclohexane (A51)

To a mixture of cis-3,5-diazidocyclohexan-1-ol (A50) (340 mg, 1.87 mmol) and KOH (387 mg, 6.91 mmol) in DMSO (8 mL) was added Mel (1.32 g, 9.33 mmol, 581 μL), and the reaction mixture was stirred at rt overnight. The reaction mixture was poured into water (50 mL), and the aqueous portion extracted with EtOAc (20 mL×3). The combined organic layers were washed with brine (20 mL×3), dried over Na₂SO₄, filtered, and concentrated to dryness to afford A51 (370 mg), which was used without further purification.

### Step 8F. Synthesis of cis-5-methoxycyclohexane-1,3-diamine (A52)

A mixture of cis-1,3-diazido-5-methoxycyclohexane (A51) (370 mg, 1.89 mmol) and Pd/C (100 mg, 50% purity) in EtOAc (5 mL)/MeOH (5 mL) was stirred at rt for 36 hrs under H₂ (15 Psi). The reaction mixture was filtered, and the filtrate concentrated to dryness to afford A52 (270 mg), which was used without further purification. ¹H NMR (400 MHz, CDCl₃) δ 3.37 (s, 3H), 3.25-3.16 (m, 1H), 2.81-2.74 (m, 2H), 2.24-1.99 (m, 3H), 1.33-1.27 (m, 3H).

### Intermediate Scheme 9: Synthesis of alkyl 3,5-diaminopiperidine-1-carboxylate (A55)

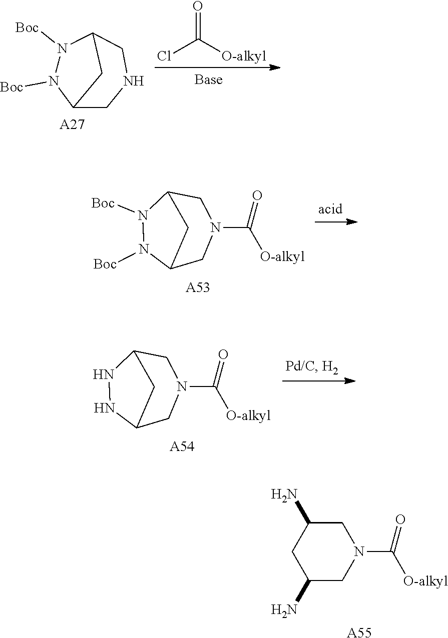

Alkyl 3,5-diaminopiperidine-1-carboxylates (A55) (relative stereochemistry at centers 3 and 5 of piperidine ring is cis) may be prepared in accordance with the above scheme by first reacting A27 with an alkyl chloroformate such as methyl chloroformate and a base such as TEA to afford a 6,7-di-tert-butyl-3-alkyl 3,6,7-triazabicyclo[3.2.1]octane-3,6,7-tricarboxylate (A53). The Boc-protecting groups can then be removed by subjecting A53 to acidic conditions such as HCl in 1,4-dioxane to afford an alkyl 3,6,7-triazabicyclo[3.2.1]octan-3-carboxylate (A54). A54 can then be reacted with Pd/C and H₂ gas to afford A55.

### Intermediate Scheme 10: Synthesis of 1-(alkylsulfonyl)piperidine-3,5-diamines (A58)

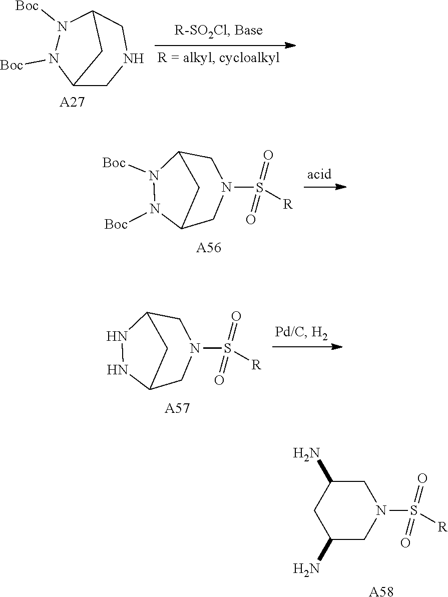

1-(alkylsulfonyl)piperidine-3,5-diamines and 1-cycloalkylsulfonyl)piperidine-3,5-diamines (A58) (relative stereochemistry at centers 3 and 5 of piperidine ring is cis) may be prepared in accordance with the above scheme by first reacting A27 with alkylsulfonylchloride or cycloalkylsulfonylchloride and a base such as TEA to afford di-tert-butyl 3-(alkylsulfonyl)-3,6,7-triazabicyclo[3.2.1]octane-6,7-dicarboxylate or di-tert-butyl 3-(cycloalkylsulfonyl)-3,6,7-triazabicyclo[3.2.1]octane-6,7-dicarboxylate (A56). The Boc-protecting groups can then be removed by subjecting A51 to acidic conditions such as HCl in 1,4-dioxane to afford 3-(alkylsulfonyl)-3,6,7-triazabicyclo[3.2.1]octane or 3-(cycloalkylsulfonyl)-3,6,7-triazabicyclo[3.2.1]octane (A57). A57 can then be reacted with Pd/C and H₂ gas to afford A58.

### Intermediate Scheme 11: Preparation of tert-butyl (3-amino-2-hydroxycycloheptyl)carbamate (relative stereochemistry is indicated in Intermediate Scheme 11)

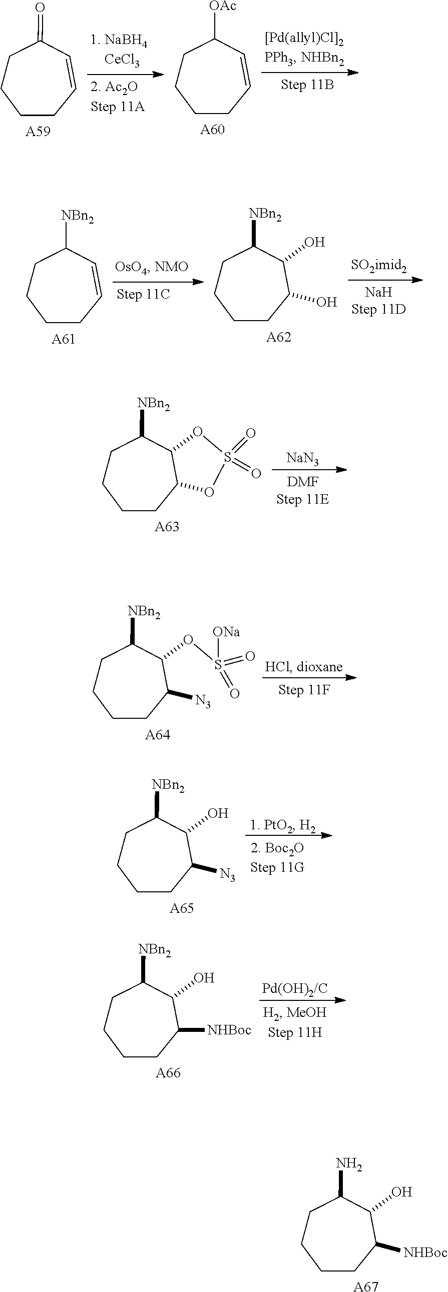

### Step 11A. Synthesis of cyclohept-2-en-1-yl acetate (A60)

To a solution of CeCl₃·7H₂O (33.8 g, 90.8 mmol) in MeOH (91 mL) was added 2-cyclohepten-1-one (A59) (11.1 g, 90.8 mmol) and stirred at 0° C. NaBH₄ (3.47 g, 90.8 mmol) was then added into the reaction mixture in portions, and the reaction mixture was stirred at rt for 16 hrs. 1 M HCl in water (100 mL) was added into the reaction mixture and the aqueous portion extracted with EtOAc (3×40 mL). The combined organic layers were washed with brine (30 mL), dried over Na₂SO₄, filtered, and concentrated. The resulting suspension was filtered and washed with DCM, and the filtrate was concentrated to give cyclohept-2-en-1-ol (10.2 g, quant.).

The cyclohept-2-en-1-ol (10.2 g, 90.8 mmol) was dissolved in DCM (100 mL), triethylamine (25.5 mL, 182 mmol) was added, and the reaction mixture was cooled to 0° C. Ac₂O (8.59 mL, 90.9 mmol) was added slowly, followed by DMAP (0.57 g, 4.55 mmol) and the reaction mixture warmed to rt. Sat. aq. NaHCO₃ solution (100 mL) was added into the reaction mixture at rt. The organic layer was washed with brine (60 mL), dried over Na₂SO₄, filtered and concentrated to dryness. The residue was purified with a silica pad to give A60 (12.9 g, 92% yield). ¹H NMR (400 MHz, CDCl₃) δ 5.85-5.78 (m, 1H), 5.65-5.61 (m, 1H), 5.40 (d, 1H), 2.24-2.16 (m, 1H), 2.12-2.07 (m, 1H), 2.05 (s, 3H), 1.96-1.81 (m, 2H), 1.72-1.59 (m, 3H), 1.44-1.35 (m, 1H)

### Step 11B. Synthesis of N,N-dibenzylcyclohep-2-en-1-amine (A61)

[Pd(allyl)₂Cl]₂ (2.13 g, 5.7 mmol) and PPh₃ (4.54 g, 17.1 mmol) in anhydrous DCM (285 mL) was degassed with N₂ for 1 hr. Cyclohept-2-en-1-yl acetate (A60) (22.0 g, 143 mmol) and Bn₂NH (56.3 mL, 287 mmol) was added, and the reaction mixture was stirred at rt for 20 hrs. Sat. aq. NH₄Cl solution (250 mL) was added into the reaction mixture at rt, and the resulting mixture was stirred for 1 hr. The organic layer was separated and filtered. The filtrate was washed with brine (150 mL), dried over Na₂SO₄, filtered and concentrated to dryness. The residue was purified with a silica pad to give A61 (26.6 g, 64% yield). ¹H NMR (400 MHz, CDCl₃) δ 7.43 (d, 4H,), 7.35-7.31 (m, 4H), 7.27-7.21 (m, 2H), 5.98 (d, 1H), 5.88-5.82 (m, 1H), 3.75 (d, 2H), 3.60 (d, 2H), 3.37 (d, 1H), 2.21-2.16 (m, 1H), 2.08-1.91 (m, 3H), 1.72-1.63 (m, 1H), 1.58-1.27 (m, 3H). LC-MS: m/z 292.2 [M+H]⁺.

### Step 11C. Synthesis of 3-(dibenzylamino)cycloheptane-1,2-diol (A62)

To a solution of N,N-dibenzylcyclohep-2-en-1-amine (A61) (25.0 g, 85.8 mmol) in 10:1 Acetone/Water (185 mL) was added OsO₄ (4% solution in water, 10 mL, 1.57 mmol) and NMO (50% solution in water, 23.1 mL, 112 mmol) and the reaction mixture stirred at rt. After 16 hrs, additional OsO₄ (4% solution in water, 5 mL, 0.79 mmol) was added and the reaction mixture stirred for an additional 24 hrs. Sat. aq. Na₂SO₃ solution (150 mL) was added into the reaction mixture and the aqueous portion was extracted with EtOAc (3×50 mL). The combined organic layers were washed with brine (100 mL), dried over Na₂SO₄, filtered and concentrated to dryness. The residue was purified by column chromatography on silica gel to give A62 (12.7 g, 46% yield, 55% yield based on recovered starting material). ¹H NMR (400 MHz, CDCl₃) δ 7.34-7.30 (m, 4H), 7.22-7.28 (m, 6H), 4.96 (s, 1H), 4.10-4.08 (m, 1H), 3.81 (d, 2H), 3.49 (dd, 1H), 3.36 (d, 2H), 2.85 (td, 1H), 2.43 (s, 1H), 2.05-1.99 (m, 1H), 1.83-1.65 (m, 3H), 1.60-1.31 (m, 4H). LC-MS: m/z 326.2 [M+H]⁺.

### Step 11D. Synthesis of 4-(dibenzylamino)hexahydro-4H-cyclohepta[d][1,3,2]dioxathiole 2,2-dioxide (A63)

To a solution of 3-(dibenzylamino)cycloheptane-1,2-diol (A62) (12.5 g, 38.5 mmol) in dry THF (250 mL) was added sulfonyl diimidazole (11.8 g, 58.5 mmol) at 0° C. A suspension of NaH (3.85 g, 96.3 mmol) in THF (50 mL) was added at 0° C., and the reaction mixture was allowed to warm to rt. After 18 hrs, the reaction mixture was cooled to 0° C. Sat. aq. NH₄Cl solution (100 mL) was slowly added, and the aqueous portion extracted with EtOAc (3×80 mL). The combined organic layers were washed with brine (100 mL), dried over Na₂SO₄, filtered and concentrated to dryness. The residue was purified by column chromatography on silica gel to give A63 (12.0 g, 80% yield). ¹H NMR (400 MHz, CDCl₃) δ 7.41-7.22 (m, 10H), 5.22 (dd, 1H), 4.79 (ddd, 1H), 3.80 (d, 2H), 3.57 (d, 2H), 3.13 (t, 1H), 2.03-2.12 (m, 1H), 2.02-1.79 (m, 4H), 1.31-1.11 (m, 3H). LC-MS: m/z 388.1 [M+H]⁺.

### Step 11E. Synthesis of sodium-2-azido-7-(dibenzylamino)cycloheptyl sulfate (A64)

To a solution of 4-(dibenzylamino)hexahydro-4H-cyclohepta[d][1,3,2]dioxathiole 2,2-dioxide (A63) (12 g, 30.9 mmol) in DMF (250 mL) was added sodium azide (4.01 g, 61.7 mmol). The reaction mixture was stirred at 50° C. for 3 hrs, and then stirred at rt for 16 hrs. Brine (150 mL) was added into the reaction mixture and the aqueous portion was extracted with EtOAc (3×60 mL). The combined organic layers were, dried over Na₂SO₄, filtered, and concentrated to dryness. The residue was purified by column chromatography on silica gel to give A64 (7.3 g, 52% yield). LC-MS: m/z 429.1 [M−Na]⁻.

### Step 11F. Synthesis of 2-azido-7-(dibenzylamino)cycloheptan-1-ol (A65)

To a solution of sodium-2-azido-7-(dibenzylamino)cycloheptyl sulfate (A64) (7.28 g, 16.1 mmol) in dioxane (86 mL) was added aq. HCl (4 M in water, 10 mL, 40 mmol) and the reaction mixture was stirred at 80° C. for 4 hrs. Sat. aq. NaHCO₃ solution (80 mL) was then added into the reaction mixture at rt, and the aqueous portion extracted with EtOAc (3×30 mL). The combined organic layers were washed with brine (80 mL), dried over Na₂SO₄, filtered, and concentrated to dryness. The residue was purified by column chromatography on silica gel to give A65 (5.38 g, 95% yield). ¹H NMR (400 MHz, CDCl₃) δ 7.35-7.24 (m, 10H), 4.94 (s, 1H), 3.83 (d, 2H), 3.50 (dd, 1H), 3.31 (d, 2H), 3.17-3.12 (m, 1H), 2.50 (t, 1H), 2.03-1.99 (m, 1H), 1.83-1.63 (m, 3H), 1.58-1.30 (m, 4H). LC-MS: m/z 351.2 [M+H]⁺.

### Step 11G. Synthesis of tert-butyl (3-(dibenzylamino)-2-hydroxycycloheptyl)carbamate (A66)

To a solution of 2-azido-7-(dibenzylamino)cycloheptan-1-ol (A65) (5.35 g, 15.3 mmol) in THF (90 mL) was added PtO₂ (0.89 g). The reaction mixture was degassed under vacuum and purged with H₂ several times and then stirred under H₂ (1 atm) at rt for 5 hrs. The reaction mixture was filtered through celite, and the filtrate was concentrated to ˜50 mL volume.

To the solution was added Boc₂O (6.34 g, 29.1 mmol), followed by triethylamine (2.6 mL, 18.4 mmol), and stirred at rt for 2 hrs. Brine (50 mL) was added into the reaction mixture and the aqueous portion was extracted with EtOAc (3×30 mL). The combined organic layers were, dried over Na₂SO₄, filtered, and concentrated to dryness. The residue was purified by column chromatography on silica gel to give A66 (4.90 g, 75% yield). ¹H NMR (400 MHz, CDCl₃) δ 7.34-7.22 (m, 10H), 4.90 (s, 1H), 4.78 (s, 1H), 3.81 (d, 2H), 3.35-3.28 (m, 3H), 3.17-3.09 (m, 1H), 2.51 (t, 1H), 2.02-1.89 (m, 2H), 1.82-1.70 (m, 1H), 1.68-1.59 (m, 1H), 1.59-1.32 (m, 4H), 1.41 (s, 9H). LC-MS: m/z 425.2 [M+H]⁺.

### Step 11H. Synthesis of (tert-butyl (3-amino-2-hydroxycycloheptyl)carbamate (A67)

To a solution of tert-butyl (3-(dibenzylamino)-2-hydroxycycloheptyl)carbamate (A66) (4.0 g, 9.42 mmol) in MeOH (35 mL) was added Pd(OH)₂/C (0.47 g). The reaction mixture was degassed under vacuum and purged with H₂ several times. The reaction mixture was stirred under H₂ (1 atm) at rt for 16 hrs. The reaction mixture was filtered, and the filtrate was concentrated to dryness to give A67 (2.22 g, 96% yield). ¹H NMR (400 MHz, CDCl₃) δ 4.78 (s, 1H), 3.50-3.39 (m, 1H), 3.01 (dd, 1H), 2.67 (dt, 1H), 2.60-1.70 (br s, 3H), 1.91-1.87 (m, 1H), 1.80-1.71 (m, 1H), 1.70-1.35 (m, 6H), 1.45 (s, 9H). LC-MS: m/z 245.2 [M+H]⁺.

### Intermediate Scheme 12: Preparation of 3,5-diaminotetrahydro-2H-thiopyran 1,1-dioxide (relative stereochemistry is indicated in Intermediate Scheme 12 for Steps 12H, 12I, and 12J)

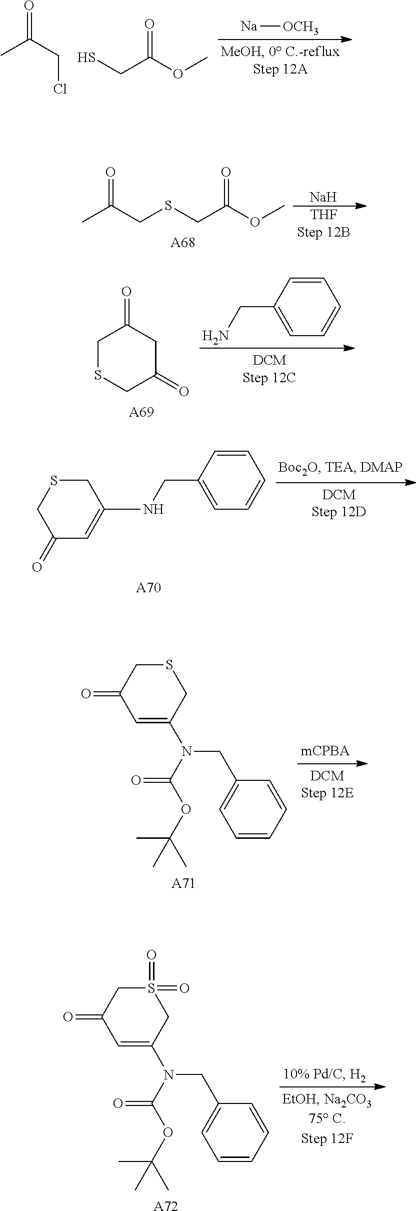

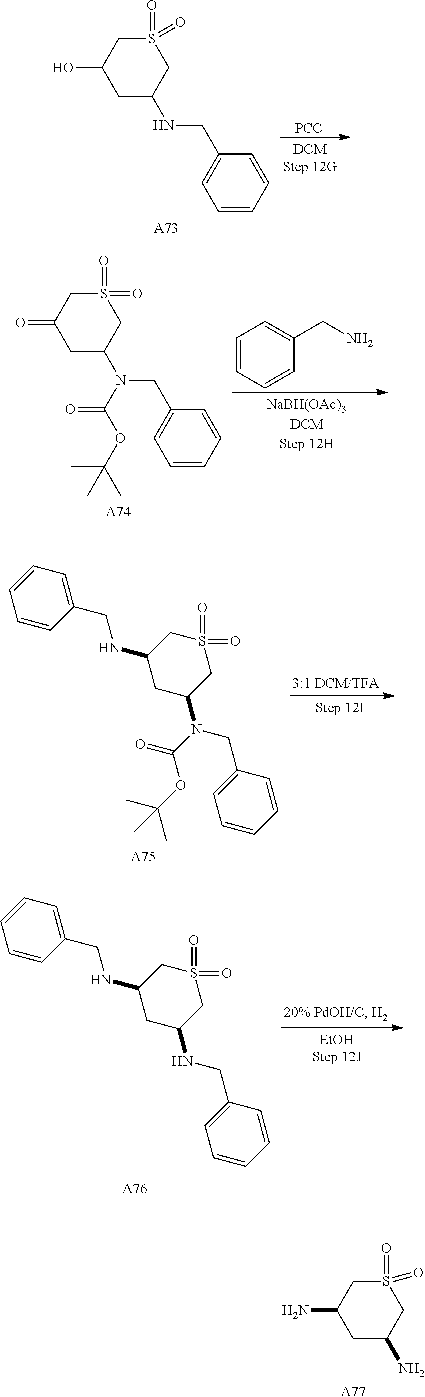

### Step 12A. Synthesis of methyl 2-((2-oxopropyl)thio)acetate (A68)

A solution of 300 mL of anhydrous methanol and 10.18 g of sodium methoxide (190 mmol) was cooled in an ice bath and then 16.9 mL of methyl thioglycolate (190 mmol) was added. After 5 minutes of stirring, to the reaction mixture was slowly added 17.4 g of chloroacetone (15 mL, 190 mmol). Upon completion of chloroacetone addition, the reaction mixture was warmed to rt for 15 minutes and then heated to 70° C. for 1 hr. The reaction mixture was cooled, filtered, and concentrated. MTBE was added, followed by water. The aqueous layer was extracted 3 times with MTBE, and the combined organic layers were washed with sat. sodium bicarbonate solution, dried over Na₂SO₄, filtered, and concentrated to dryness to afford 26.72 g of A68, which was used without further purification. ¹H NMR (400 MHz, CDCl₃) δ 3.73 (d, 3H), 3.42 (s, 2H), 3.26 (s, 2H), 2.28 (s, 3H).

### Step 12B. Synthesis of 2H-thiopyran-3,5(4H,6H)-dione (A69)

A solution of 8.33 g of sodium hydride (60% dispersion in oil, 210 mmol) in 100 mL of anhydrous THF was cooled in an ice water bath and then 26.72 g (165 mmol) of methyl 2-((2-oxopropyl)thio)acetate (A68) as a solution in 105 mL of THF was slowly added to the reaction mixture via cannula. The reaction mixture was stirred for several hours. Water (150 mL) was added, and the aqueous portion extracted several times with MTBE. The aqueous layer was acidified to a pH between 1.5-2.5 and extracted several times with DCM. The combined organic layers were dried over Na₂SO₄, filtered, and concentrated to dryness. The residue was dissolved in minimal MTBE with minimal DCM and placed in the −20° C. fridge for two days and then the material was washed once again with MTBE containing heptanes. The material was decanted and then dried to afford 12.73 grams of A69, which was used without further purification (59.4% yield). LC-MS: m/z 131.0 [M+H]⁺.

### Step 12C. Synthesis of 5-(benzylamino)-2H-thiopyran-3(6H)-one (A70)

A solution of 14.05 g of 2H-thiopyran-3,5(4H,6H)-dione (A69) (108 mmol) in 300 mL of DCM was cooled in an ice bath and then 11.8 mL of benzyl amine (108 mmol) were added, and the reaction mixture allowed to stir for 20 hrs. Precipitation was observed during the initial stages of the reaction. After the 20 hrs, the precipitate was separated from the reaction mixture using a fritted filter funnel and washed with DCM several times. The precipitate was dried to afford 10.37 g of A70. The filtrate solution's solvent was concentrated to dryness, and the residue was purified by column chromatography (330 g normal phase isco column; eluent 25-100% EtOAc:heptanes) to afford 3.5 grams of A70 (58.6% total yield from precipitation and column chromatography). ¹H NMR (400 MHz, DMSO-d₆) δ 7.69 (s, 1H), 7.39-7.24 (m, 2H), 4.82 (s, 1H), 4.23 (d, 1H), 3.44 (s, 1H), 3.09 (s, 1H). LC-MS: m/z 220.1 [M+H]⁺.

### Step 12D. Synthesis of tert-butyl benzyl(5-oxo-5,6-dihydro-2H-thiopyran-3-yl)carbamate (A71)

To a solution of 5.94 g of 5-(benzylamino)-2H-thiopyran-3(6H)-one (A70) (27.1 mmol) in 100 mL DCM, 5.7 mL of triethyl amine (4.14 g, 41 mmol), 662 mg of DMAP (5.42 mmol) and 8.57 g of Boc anhydride (39.3 mmol) were added. The flask was flushed with argon, and the reaction mixture allowed to stir overnight at rt and then concentrated to dryness. The residue was purified by column chromatography (normal phase isco column; eluent 0-25% EtOAc:heptanes) to afford 2.86 g of A71 (38.1% yield). LC-MS: m/z 264.1 [M+H⁺-Tbutyl].

### Step 12E. Synthesis of tert-butyl benzyl(1,1-dioxido-5-oxo-5,6-dihydro-2H-thiopyran-3-yl)carbamate (A72)

A solution of 4.29 g of tert-butyl benzyl(5-oxo-5,6-dihydro-2H-thiopyran-3-yl)carbamate (A71) (13.4 mmol) in 300 mL DCM was cooled in an ice water bath. Then, 7.52 g mCPBA (33.6 mmol; 77% purity) was added portion wise. After 15 minutes the reaction mixture was allowed to warm to rt and stirred for an additional 2 hrs. The reaction mixture was quenched by the addition of solid sodium sulfite and solid sodium bicarbonate, and then sat. aq. sodium bicarbonate solution, sat. aq. sodium sulfite, and deionized water were added. The aqueous portion was extracted with DCM (2×), and the combined organic layers dried over Na₂SO₄, filtered, and concentrated to dryness. The residue was purified by column chromatography (80 g normal phase isco gold column; eluent 0-45% EtOAc:heptanes) to afford 4.26 g of A72 (90.3% yield). LC-MS: m/z 350.1 [M−H]⁺.

### Step 12F. Synthesis of tert-butyl benzyl(5-hydroxy-1,1-dioxidotetrahydro-2H-thiopyran-3-yl)carbamate (A73)

9.4 g of tert-butyl benzyl(1,1-dioxido-5-oxo-5,6-dihydro-2H-thiopyran-3-yl)carbamate (A72) (26.7 mmol) and 2.84 g of sodium carbonate (26.8 mmol) were dissolved/suspended in 450 mL of 200 proof EtOH and 5.5 g of 10% Pd/C were added. The reaction mixture was sparged with hydrogen, and the flask flushed with hydrogen gas. The reaction mixture was then heated to 75° C. under a hydrogen atmosphere overnight. The reaction mixture was then filtered, and the filtrate concentrated. The residue was dry loaded onto silica gel, and purified by column chromatography (normal phase isco column, gradient 0-50% ethyl acetate in heptanes) to afford 798 mg of A73 (8.39% yield). LC-MS: m/z 256.1 [M+H⁺-BOC].

### Step 12G. Synthesis of tert-butyl benzyl(1,1-dioxido-5-oxotetrahydro-2H-thiopyran-3-yl)carbamate (A74)

760 mg (2.14 mmol) of tert-butyl benzyl(5-hydroxy-1,1-dioxidotetrahydro-2H-thiopyran-3-yl)carbamate (A73), PCC (553 mg, 2.56 mmol) and 100 mg of crushed 4A molecular sieves were dissolved/suspended in 7 mL of dry DCM, and the reaction mixture stirred at rt for 3 hrs. After 2.5 hrs, additional PCC (˜0.5 eq) was added and within 30 minutes LC-MS showed full consumption of starting material. The reaction mixture was passed through a plug of silica using 40% ethyl acetate in heptanes to afford 610 mg of A74 (80.7% yield), which was used without further purification. LC-MS: m/z 352.2 [M−H⁺].

### Step 12H. Synthesis of tert-butyl benzyl(5-(benzylamino)-1,1-dioxidotetrahydro-2H-thiopyran-3-yl)carbamate (A75)

To a solution of tert-butyl benzyl(1,1-dioxido-5-oxotetrahydro-2H-thiopyran-3-yl)carbamate (A74) (610 mg, 1.73 mmol) in 9 mL of DCM was added 185 mg (188 μL, 1.73 mmol) of benzyl amine. The reaction mixture was allowed to stir for 1 hr and 45 min and then sodium triacetoxy borohydride was added (733 mg, 3.46 mmol). The reaction mixture was then allowed to stir overnight. Additional aliquots of benzyl amine and sodium triacetoxy borohydride were added until all the starting material was consumed by LC-MS. After reaction completion, sat. aq. sodium bicarbonate solution was added, and the aqueous portion extracted several times with EtOAc. The combined organic extracts were concentrated to dryness, and the residue held under high vacuum for several hours. The residue was then dissolved in DCM and passed through a silica plug which had been neutralized with Hüing's base (eluent EtOAc) to give 620 mg of A75 (80.8% yield), which was used without further purification. LC-MS: m/z 445.2 [M+H⁺].

### Step 121. Synthesis of 3,5-bis(benzylamino)tetrahydro-2H-thiopyran 1,1-dioxide (A76)

620 mg (1.39 mmol) of tert-butyl benzyl-5-(benzylamino)-1,1-dioxidotetrahydro-2H-thiopyran-3-yl)carbamate (A75) was dissolved in 30 mL of 20% TFA in DCM and allowed to stir at rt until LC-MS showed complete loss of the Boc protecting group. The reaction mixture was then quenched by adding solid sodium carbonate followed by minimal sat. aq. sodium bicarbonate solution. The aqueous portion was extracted with EtOAc, and the organic layer concentrated to dryness. The residue was dissolved in DCM and passed through a silica plug (eluent EtOAc) and concentrated to dryness to afford A76, which was used without further purification. LC-MS: m/z 345.2 [M+H⁺].

### Step 12J. Synthesis of 3,5-diaminotetrahydro-2H-thiopyran 1,1-dioxide (A77)

3,5-bis(benzylamino)tetrahydro-2H-thiopyran 1,1-dioxide (A76) was dissolved in 8 ml of methanol and 250 mg of 20% PdOH/C was added. The reaction mixture was then flushed with hydrogen and kept under an atmosphere of hydrogen via a hydrogen balloon and heated to 80° C. overnight. The reaction mixture was cooled and passed through a celite plug after LC-MS indicated that the hydrogenolysis was complete. The celite was washed with MeOH and EtOAc. The organic portion were concentrated to dryness to afford A77, which was used without further purification. ¹H NMR (400 MHz, CD₃OD) δ 3.58-3.51 (m, 1H), 3.26 (ddd, 1H), 2.97 (ddd, 1H), 1.84 (t, 1H); LC-MS: m/z 165.0 [M+H⁺].

### Intermediate Scheme 13: Preparation of benzyl tert-butyl (5-cyanocyclohexane-1,3-diyl)dicarbamate (Relative Stereochemistry is Indicated in Intermediate Scheme 13)

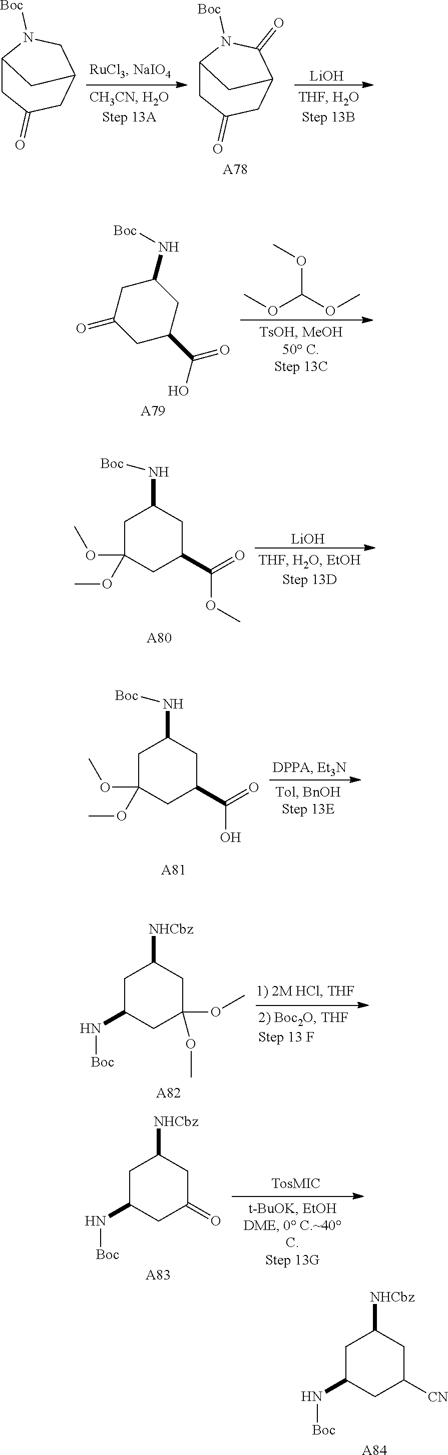

### Step 13A. Synthesis of tert-butyl 3,7-dioxo-6-azabicyclo[3.2.1]octane-6-carboxylate (A78)

To a solution of tert-butyl 3-oxo-6-azabicyclo[3.2.1]octane-6-carboxylate (3.2 g, 14.2 mmol) in EtOAc (20 mL) was added a solution of RuCl₃·H₂O (160 mg, 710 μmol) and NaIO₄ (12.15 g, 56.8 mmol, 3.15 mL) in H₂O (15 mL). The reaction mixture was stirred at rt for 18 hrs. Isopropanol (6 mL) was added, and the reaction mixture was stirred at rt for another 20 min and concentrated to dryness. The residue was dissolved in EtOAc (100 mL), washed with water (100 mL) and brine (100 mL), dried over Na₂SO₄, filtered, and concentrated to dryness. The residue was purified by flash silica gel chromatography to give A78 (2.8 g, 82.4% yield). ¹H NMR (400 MHz, CDCl₃) δ 4.59-4.58 (m, 1H), 2.92-2.90 (m, 2H), 2.78-2.76 (m, 1H), 2.64-2.51 (m, 3H), 1.94-1.91 (m, 1H), 1.53 (s, 9H).

### Step 13B. Synthesis of 3-((tert-butoxycarbonyl)amino)-5-oxocyclohexane-1-carboxylic acid (A79)

To a mixture of tert-butyl 3,7-dioxo-6-azabicyclo[3.2.1]octane-6-carboxylate (A78) (2.8 g, 11.7 mmol) in THF (30 mL)/H₂O (20 mL) was added LiOH·H₂O (1.23 g, 29.3 mmol), and the reaction mixture was stirred at rt for 18 hrs. Water (50 mL) was added to the reaction mixture, and the pH was adjusted to 3 by the addition of 0.5 M HCl. The aqueous portion was extracted with EtOAc (40 mL×3). The combined organic layers were washed with brine (40 mL×2), dried over Na₂SO₄, filtered, and concentrated to dryness to give A79 (3.0 g, 99.6% yield), which was used without further purification. ¹H NMR (400 MHz, CDCl₃) δ 4.62 (br s, 1H), 3.87 (br s, 1H), 2.78-2.73 (m, 2H), 2.64-2.62 (m, 1H), 2.52-2.46 (m, 2H), 2.28-2.23 (m, 1H), 1.46 (s, 9H).

### Step 13C. Synthesis of methyl-5-((tert-butoxycarbonyl)amino)-3,3-dimethoxycyclohexane-1-carboxylate (A80)

A mixture of 3-(tert-butoxycarbonylamino)-5-oxo-cyclohexanecarboxylic acid (A79) (3.0 g, 11.7 mmol), trimethoxymethane (4.95 g, 46.6 mmol, 5.11 mL) and TsOH (201 mg, 1.17 mmol) in MeOH (40 mL) was stirred at 50° C. for 18 hrs. The reaction mixture was concentrated to dryness. The residue was purified by flash silica gel chromatography to give A80 (2.5 g, 67.6% yield). ¹H NMR (400 MHz, CDCl₃) δ 4.41-4.39 (m, 1H), 3.68 (s, 3H), 3.65 (br s, 1H), 3.22 (s, 3H), 3.20 (s, 3H), 2.66-2.61 (m, 1H), 2.37-2.27 (m, 3H), 1.46 (s, 9H), 1.41-1.37 (m, 1H), 1.26-1.09 (m, 2H); LC-MS: m/z 340.1 (M+Na)⁺.

### Step 13D. Synthesis of 5-((tert-butoxycarbonyl)amino)-3,3-dimethoxycyclohexane-1-carboxylic acid (A81)

A mixture of methyl-5-((tert-butoxycarbonyl)amino)-3,3-dimethoxycyclohexane-1-carboxylate (A80) (1.8 g, 5.67 mmol) and LiOH·H₂O (1.19 g, 28.4 mmol) in THF (8 mL)/H₂O (8 mL)/EtOH (2 mL) was stirred at rt for 18 hrs. Water (50 mL) was added to the reaction mixture, and the pH adjusted to 4 by adding aq. citric acid solution. The aqueous portion was extracted with EtOAc (25 mL×3). The combined organic layers were washed with brine (20 mL×3), dried over Na₂SO₄, filtered, and concentrated to dryness to give A81 (1.7 g), which was used without further purification. ¹H NMR (400 MHz, CDCl₃) δ 4.44-4.42 (m, 1H), 3.65 (br s, 1H), 3.23 (s, 3H), 3.20 (s, 3H), 2.68-2.62 (m, 1H), 2.37-2.24 (m, 3H), 1.45 (s, 9H), 1.39-1.21 (m, 3H).

### Step 13E. Synthesis of benzyl tert-butyl (5,5-dimethoxycyclohexane-1,3-diyl)dicarbamate (A82)

To a solution of 5-((tert-butoxycarbonyl)amino)-3,3-dimethoxycyclohexane-1-carboxylic acid (A81) (2.3 g, 7.58 mmol) and Et₃N (1.15 g, 11.4 mmol, 1.58 mL) in toluene (35 mL) was added DPPA (2.71 g, 9.86 mmol, 2.14 mL), and the reaction mixture was stirred at rt for 2 hrs. Then BnOH (1.64 g, 15.2 mmol, 1.58 mL) was added and the reaction mixture was stirred at 100° C. for 18 hrs. The reaction mixture was concentrated to dryness, and the residue was purified by flash silica gel chromatography to give A82 (2.2 g, 71.0% yield). LC-MS: 431.0 (M+Na)⁺.

### Step 13F. Synthesis of benzyl tert-butyl (5-oxocyclohexane-1,3-diyl)dicarbamate (A83)

To a solution of tert-butyl N-[5-(benzyloxycarbonylamino)-3,3-dimethoxy-cyclohexyl]carbamate (A82) (2.2 g, 5.39 mmol) in THF (20 mL) was added HCl (2 M, 8.08 mL), and the reaction mixture was stirred at 60° C. for 18 hrs. THF (10 mL) was added to the reaction mixture, and pH adjusted to 8 by adding sat. aq. Na₂CO₃ solution. Boc₂O (2.35 g, 10.77 mmol, 2.47 mL) was added, and the reaction mixture was stirred at rt for 2 hrs. Water (50 mL) was added, and the aqueous portion extracted with EtOAc (25 mL×3). The combined organic layers were washed with brine (25 mL), dried over Na₂SO₄, filtered, and concentrated to dryness. The residue was purified by flash silica gel chromatography to give A83 (1.5 g, 76.9% yield). ¹H NMR (400 MHz, CDCl₃) δ 7.41-7.33 (m, 5H), 5.12 (s, 2H), 4.83 (br s, 1H), 4.55 (br s, 1H), 3.89-3.83 (m, 2H), 2.79-2.72 (m, 2H), 2.51-2.48 (m, 1H), 2.28-2.22 (m, 2H), 1.68 (br s, 1H), 1.46 (s, 9H); LC-MS: 484.2 (M+Na)*.

### Step 13G. Synthesis of benzyl tert-butyl (5-cyanocyclohexane-1,3-diyl)dicarbamate (A84)

To a solution of benzyl tert-butyl (5-oxocyclohexane-1,3-diyl)dicarbamate (A83) (1.5 g, 4.14 mmol), 1-(isocyanomethylsulfonyl)-4-methyl-benzene (970 mg, 4.97 mmol) and EtOH (229 mg, 4.97 mmol) in DME (10 mL) was added t-BuOK (604 mg, 5.38 mmol) at 0° C., and the reaction mixture was stirred at 0° C. to rt for 1 hr. The reaction mixture was then heated to 40° C. and stirred for 18 hrs. The reaction mixture was then concentrated to dryness to give a residue. The residue was purified by silica gel chromatography to give A84 (370 mg, 23.9% yield). LC-MS: 396.0 (M+Na)⁺.

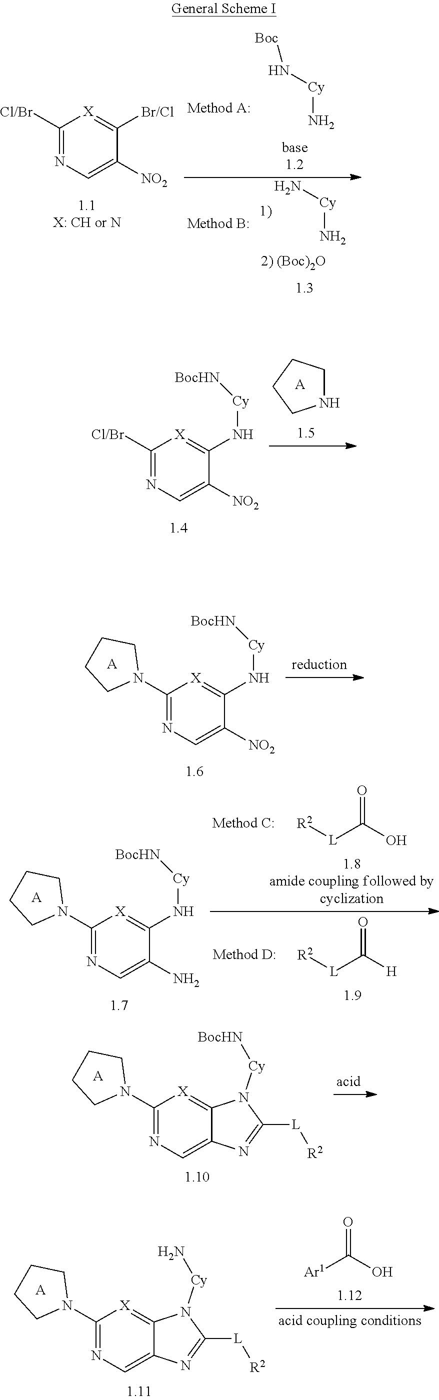

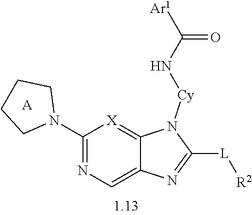

Compounds of formula 1.13 were obtained through General Scheme I. Beginning with the 2,4-di-halo-5-nitropyridine 1.1, a nucleophilic aromatic substitution reaction conducted under basic conditions was used to couple a N-Boc-1,3-Cy-diamine compound of formula 1.2 (Method A) to afford compounds of formula 1.4. Alternatively, reagent 1.1 underwent selective nucleophilic aromatic substitution by 1,3-Cy-diamine of formula 1.3 (Method B), and the intermediate amine group was protected with a Boc-protecting group (or other suitable nitrogen protecting group) to afford compounds of formula 1.4. The R¹ five-membered heteroaryl or heterocycloalkyl containing at least one nitrogen was installed by displacing the bromide or chloride in a compound of formula 1.4 with the NH of the heteroaryl or heterocycloalkyl reagent 1.5 in the presence of a suitable base such as cesium carbonate and/or a metal catalyst such as CuI to form compounds of formula 1.6. The nitro group in a compound of formula 1.6 was reduced with a reducing agent, such as iron in the presence of ammonium chloride or Pd—C, to afford the pyridine-diamine compound of formula 1.7. Subsequently, various L and R² groups were installed by coupling the pyridine-diamine compound of formula 1.7 with an acid of formula 1.8 to form an amide intermediate (Method C), which was then cyclized under either basic or acidic conditions to form compounds of formula 1.10. Alternatively, the pyridine-diamine compound of formula 1.7 was condensed with an aldehyde of formula 1.9 (Method D), followed by cyclization and in situ oxidation to afford compounds of formula 1.10. The Boc protecting group of a compound of formula 1.10 was then removed under acidic conditions to form compounds of formula 1.11. Ar¹ groups were then installed by reacting an Ar¹ carboxylic acid of formula 1.12 or a basic salt (i.e., Li, K, or Na) thereof with the amine of formula 1.11 using acid coupling conditions known in the art, such as using one of the following reagents—HATU, EDCI, HOBt—along with a base, such as DIPEA (Hunig's base) or TEA to afford compounds of formula 1.13, wherein R¹ is a five-membered heteroaryl or heterocycloalkyl containing at least one nitrogen.

### Preparation of Example 25 Via General Scheme I (Methods A and D)

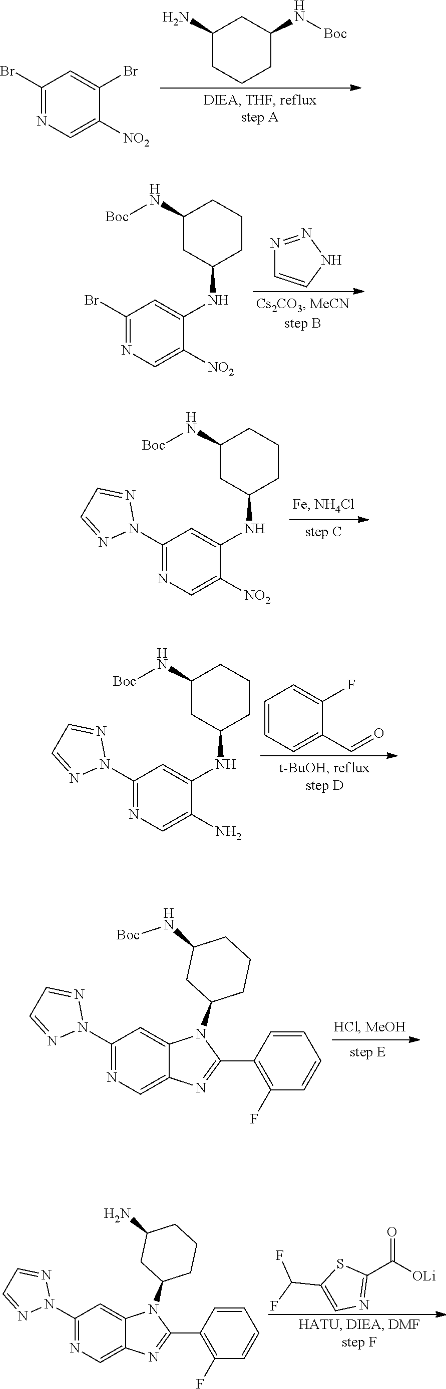

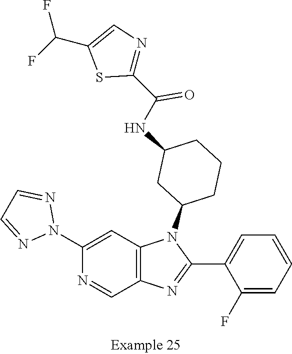 [SMILES: O=C(N[C@H]1CCC[C@@H](n2c(-c3ccccc3F)nc3cnc(-n4nccn4)cc32)C1)c1ncc(C(F)F)s1]

### Step A. Synthesis of tert-butyl ((1S,3R)-3-((2-bromo-5-nitropyridin-4-yl)amino)cyclohexyl)carbamate

To a solution of 2,4-dibromo-5-nitro-pyridine (3.95 g, 14.0 mmol) and tert-butyl ((1S,3R)-3-aminocyclohexyl)carbamate (3.0 g, 14.0 mmol) in THF (45 mL) was added DIEA (5.43 g, 42.0 mmol). The reaction mixture was stirred at 70° C. for 15 hrs, then concentrated to dryness to give tert-butyl ((1S,3R)-3-((2-bromo-5-nitropyridin-4-yl)amino)cyclohexyl)carbamate (9 g), which was used without further purification. LC-MS: m/z 417.1 [M+H]⁺.

### Step B. Synthesis of tert-butyl ((1S,3R)-3-((5-nitro-2-(2H-1,2,3-triazol-2-yl)pyridin-4-yl)amino)cyclohexyl)carbamate

To a solution of tert-butyl ((1S,3R)-3-((2-bromo-5-nitropyridin-4-yl)amino)cyclohexyl)carbamate (1.1 g, 2.65 mmol) and 1H-triazole (220 mg, 3.18 mmol) in MeCN (14 mL) was added Cs₂CO₃ (1.73 g, 5.30 mmol). The reaction mixture was stirred at 80° C. for 15 hrs, then allowed to cool. The resulting slurry was filtered, and the filtrate was concentrated to give a residue. The residue was purified by flash silica gel chromatography to give (1) tert-butyl ((1S,3R)-3-((5-nitro-2-(1H-1,2,3-triazol-1-yl)pyridin-4-yl)amino)cyclohexyl)carbamate (710 mg, 33.2% yield). LC-MS: m/z 404.2 [M+H]⁺. and (2) tert-butyl ((1S,3R)-3-((5-nitro-2-(2H-1,2,3-triazol-2-yl)pyridin-4-yl)amino)cyclohexyl)carbamate (770 mg, 36.0% yield). ¹H NMR (400 MHz, CDCl₃) δ 9.25 (s, 1H), 8.31 (d, 1H), 7.95 (s, 2H), 7.53 (s, 1H), 4.49 (br s, 1H), 3.76-3.68 (m, 2H), 2.51-2.47 (m, 1H), 2.20-2.16 (m, 1H), 2.06-2.02 (m, 1H), 1.94-1.92 (m, 1H), 1.54-1.51 (m, 1H), 1.45 (s, 9H), 1.29-1.16 (m, 3H). LC-MS: m/z 404.2 [M+H]⁺.

### Step C. Synthesis of tert-butyl ((1S,3R)-3-((5-amino-2-(2H-1,2,3-triazol-2-yl)pyridin-4-yl)amino)cyclohexyl)carbamate

To a solution of tert-butyl ((1S,3R)-3-((5-nitro-2-(2H-1,2,3-triazol-2-yl)pyridin-4-yl)amino)cyclohexyl)carbamate (770 mg, 1.91 mmol) in THF (8 mL) were added Fe (533 mg, 9.54 mmol) and NH₄Cl (123 mg, 2.29 mmol) in water (4 mL). The reaction mixture was stirred at 70° C. for 15 hrs, then allowed to cool. The reaction mixture was filtered, and the filtrate concentrated to dryness to give tert-butyl ((1S,3R)-3-((5-amino-2-(2H-1,2,3-triazol-2-yl)pyridin-4-yl)amino)cyclohexyl)carbamate (750 mg), which was used without further purification. LC-MS: m/z 374.2 [M+H]⁺.

### Step D. Synthesis of tert-butyl ((1S,3R)-3-(2-(2-fluorophenyl)-6-(2H-1,2,3-triazol-2-yl)-1H-imidazo[4,5-c]pyridin-1-yl)cyclohexyl)carbamate

To a solution of tert-butyl ((1S,3R)-3-((5-amino-2-(2H-1,2,3-triazol-2-yl)pyridin-4-yl)amino)cyclohexyl)carbamate (100 mg, 268 μmol) in t-BuOH (3 mL) was added 2-fluorobenzaldehyde (39.9 mg, 321 μmol) at rt. The reaction mixture was stirred at 90° C. for 16 hrs, then allowed to cool. The reaction mixture was concentrated to dryness to give a residue, which was purified by flash silica gel chromatography to give tert-butyl ((1S,3R)-3-(2-(2-fluorophenyl)-6-(2H-1,2,3-triazol-2-yl)-1H-imidazo[4,5-c]pyridin-1-yl)cyclohexyl)carbamate (60 mg, 42.2% yield). LC-MS: m/z 478.2 [M+H]⁺.

### Step E. Synthesis of (1S,3R)-3-(2-(2-fluorophenyl)-6-(2H-1,2,3-triazol-2-yl)-1H-imidazo[4,5-c]pyridin-1-yl)cyclohexan-1-amine

To a solution of tert-butyl ((1S,3R)-3-(2-(2-fluorophenyl)-6-(2H-1,2,3-triazol-2-yl)-1H-imidazo[4,5-c]pyridin-1-yl)cyclohexyl)carbamate (60 mg, 113 μmol) in MeOH (1 mL) was added HCl/dioxane (4M, 2 mL). The reaction mixture was stirred at rt for 1 hr, and then concentrated to dryness to give (1S,3R)-3-(2-(2-fluorophenyl)-6-(2H-1,2,3-triazol-2-yl)-1H-imidazo[4,5-c]pyridin-1-yl)cyclohexan-1-amine (50 mg, 2HCl salt), which was used without further purification. LC-MS: m/z 378.2 [M+H]⁺.

### Step F. Synthesis of 5-(difluoromethyl)-N-((1S,3R)-3-(2-(2-fluorophenyl)-6-(2H-1,2,3-triazol-2-yl)-1H-imidazo[4,5-c]pyridin-1-yl)cyclohexyl)thiazole-2-carboxamide

### Example 25

To a solution of (1S,3R)-3-[2-(2-fluorophenyl)-6-(triazol-2-yl)imidazo[4,5-c]pyridin-1-yl]cyclohexanamine (50 mg, 111 μmol, 2HCl salt) and [5-(difluoromethyl)thiazole-2-carbonyl]oxylithium (24.7 mg, 133 μmol) in DMF (1 mL) were added HATU (50.7 mg, 133 μmol) and DIEA (71.8 mg, 555 μmol). The reaction mixture was stirred at rt for 12 hrs, and then purified by prep-HPLC (column: Boston Prime C18 150*30 mm*5 um; mobile phase: [water (0.05% NH₃H₂O+10m M NH₄HCO₃)-ACN]; B %: 45%-75%, 7 min) to give Example 25 (16.7 mg, 27.9% yield). ¹H NMR (400 MHz, CD₃OD) δ 8.96 (s, 1H), 8.53 (s, 1H), 8.15 (s, 1H), 8.10 (s, 2H), 7.78-7.72 (m, 2H), 7.53-7.44 (m, 2H), 7.18 (t, 1H), 4.39-4.33 (m, 1H), 4.06-4.00 (m, 1H), 2.61-2.52 (m, 1H), 2.34-2.28 (m, 2H), 2.11-2.05 (m, 3H), 1.69-1.51 (m, 2H). LC-MS: m/z 539.1 [M+H]⁺.

### Preparation of Example 54 Via General Scheme I (Method D)

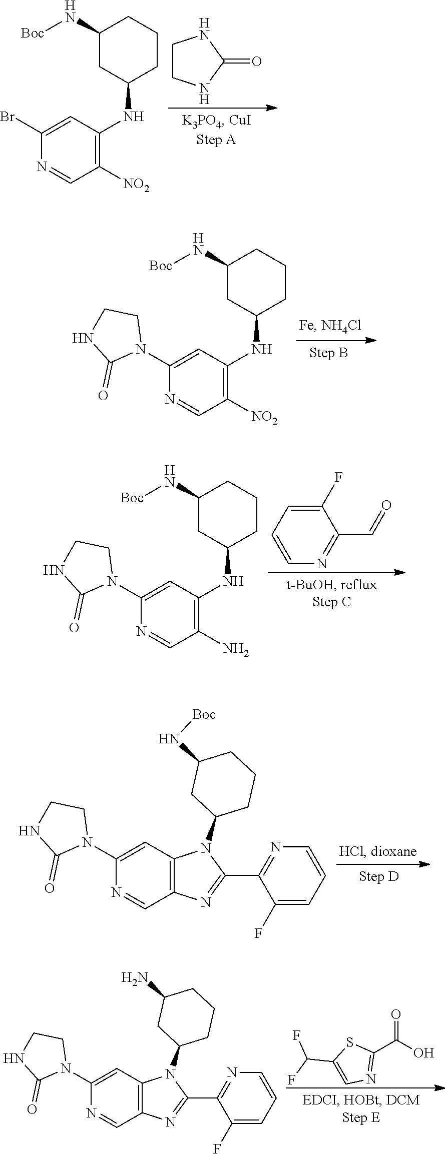

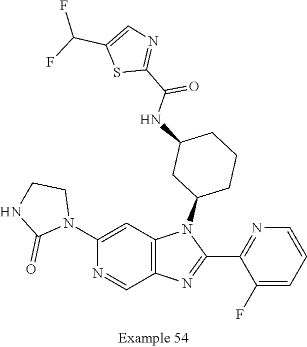 [SMILES: O=C(N[C@H]1CCC[C@@H](n2c(-c3ncccc3F)nc3cnc(N4CCNC4=O)cc32)C1)c1ncc(C(F)F)s1]

### Step A. Synthesis of tert-butyl ((1S,3R)-3-((5-nitro-2-(2-oxoimidazolidin-1-yl)pyridin-4-yl)amino)cyclohexyl)carbamate

To a mixture of tert-butyl ((1S,3R)-3-((2-bromo-5-nitropyridin-4-yl)amino)cyclohexyl) carbamate (0.1 g, 241 μmol), N,N′-dimethylethane-1,2-diamine (4.25 mg, 48.2 μmol), K₃PO₄ (102 mg, 482 μmol) and imidazolidin-2-one (20.7 mg, 241 μmol) in toluene (5 mL) was added CuI (4.59 mg, 24.1 μmol). After the addition was complete, the reaction mixture was heated at 100° C. for 16 hrs, and then allowed to cool. The reaction mixture was quenched by the addition of water (25 mL), and the aqueous portion extracted with EtOAc (3×25 mL). The combined organic layers were washed with brine (25 mL), dried over Na₂SO₄, filtered, and concentrated to dryness. The residue was purified by silica gel column chromatography to give tert-butyl ((1S,3R)-3-((5-nitro-2-(2-oxoimidazolidin-1-yl)pyridin-4-yl)amino)cyclohexyl)carbamate (65 mg). LC-MS: m/z 421.3 [M+H]⁺.

### Step B. Synthesis of tert-butyl ((1S,3R)-3-((5-amino-2-(2-oxoimidazolidin-1-yl)pyridin-4-yl)amino)cyclohexyl)carbamate

To a suspension of tert-butyl ((1S,3R)-3-((5-nitro-2-(2-oxoimidazolidin-1-yl)pyridin-4-yl)amino)cyclohexyl)carbamate (65 mg, 155 μmol) and Fe (43.2 mg, 773 μmol) in THF (6 mL) was added NH₄Cl (9.92 mg, 186 μmol) in H₂O (3 mL). The reaction mixture was stirred at 70° C. for 16 hrs, and then allowed to cool. The resulting slurry was filtered, and the filtrate was concentrated to dryness to give tert-butyl ((1S,3R)-3-((5-amino-2-(2-oxoimidazolidin-1-yl)pyridin-4-yl)amino)cyclohexyl)carbamate (55 mg) which was used without further purification. LC-MS: m/z 391.2 [M+H]⁺.

### Step C. Synthesis of tert-butyl ((1S,3R)-3-(2-(3-fluoropyridin-2-yl)-6-(2-oxoimidazolidin-1-yl)-1H-imidazo[4,5-c]pyridin-1-yl)cyclohexyl)carbamate

To a solution of tert-butyl ((1S,3R)-3-((5-amino-2-(2-oxoimidazolidin-1-yl)pyridin-4-yl)amino)cyclohexyl)carbamate (55 mg, 141 μmol) in t-BuOH (2 mL) was added 3-fluoropyridine-2-carbaldehyde (17.6 mg, 141 μmol). The reaction mixture was heated at 100° C. for 16 hrs and then allowed to cool. The resulting slurry was filtered, and the filtrate was concentrated to dryness. The residue was purified by column chromatography on silica gel to give tert-butyl ((1S,3R)-3-(2-(3-fluoropyridin-2-yl)-6-(2-oxoimidazolidin-1-yl)-1H-imidazo[4,5-c]pyridin-1-yl)cyclohexyl)carbamate (20 mg). LC-MS: m/z 496.3 [M+H]+.

### Step D. Synthesis of 1-(1-((1R,3S)-3-aminocyclohexyl)-2-(3-fluoropyridin-2-yl)-1H-imidazo[4,5-c]pyridin-6-yl)imidazolidin-2-one

To a solution of tert-butyl ((1S,3R)-3-(2-(3-fluoropyridin-2-yl)-6-(2-oxoimidazolidin-1-yl)-1H-imidazo[4,5-c]pyridin-1-yl)cyclohexyl)carbamate (20 mg, 40 μmol) in dioxane (1 mL) was added HCl/dioxane (4 M, 1 mL). After the addition was complete, the reaction mixture was stirred at rt for 30 min. The reaction mixture was concentrated to dryness to give 1-(1-((1R,3S)-3-aminocyclohexyl)-2-(3-fluoropyridin-2-yl)-1H-imidazo[4,5-c]pyridin-6-yl)imidazolidin-2-one (20 mg, HCl salt), which was used without further purification. LC-MS: m/z 496.3 [M+H]⁺.

### Step E. Synthesis of 5-(difluoromethyl)-N-((1S,3R)-3-(2-(3-fluoropyridin-2-yl)-6-(2-oxoimidazolidin-1-yl)-1H-imidazo[4,5-c]pyridin-1-yl)cyclohexyl)thiazole-2-carboxamide (Example 54)

To a solution of 1-(1-((1R,3S)-3-aminocyclohexyl)-2-(3-fluoropyridin-2-yl)-1H-imidazo[4,5-c]pyridin-6-yl)imidazolidin-2-one (20 mg, 46 μmol, HCl salt), 5-(difluoromethyl)thiazole-2-carboxylic acid (8.25 mg, 44.3 μmol, Li salt), HOBt (12.5 mg, 92.6 μmol) and EDCI (17.8 mg, 92.6 μmol) in DMF (10 mL) was added TEA (9.37 mg, 92.6 μmol). The reaction mixture was stirred at rt for 16 hrs. The resulting slurry was filtered, and the filtrate was purified by prep-HPLC (column: Welch Xtimate C18 150*30 mm*5 um; mobile phase: [water (0.050% NH₃H₂O+10 mM NH₄HCO₃)-ACN]; B %: 20%-50%, 9 min.) to give Example 54 (3 mg, 10.4% yield).

¹H NMR (400 MHz, CD₃OD) δ 8.74 (s, 1H), 8.66 (d, 1H), 8.61 (s, 1H), 8.13 (s, 1H), 7.92-7.88 (m, 1H), 7.72-7.70 (m, 1H), 7.15 (t, 1H), 4.67-4.61 (m, 1H), 4.26-4.22 (m, 2H), 4.04-3.99 (m, 1H), 3.59-3.54 (m, 2H), 2.48-2.44 (m, 1H), 2.30-2.26 (m, 2H), 2.04-1.98 (m, 3H), 1.61-1.51 (m, 2H); LC-MS: m/z 557.0 [M+H]⁺.

### Preparation of Example 112 Via General Scheme I (Methods a and D) (Relative Stereochemistry is Indicated in Scheme)

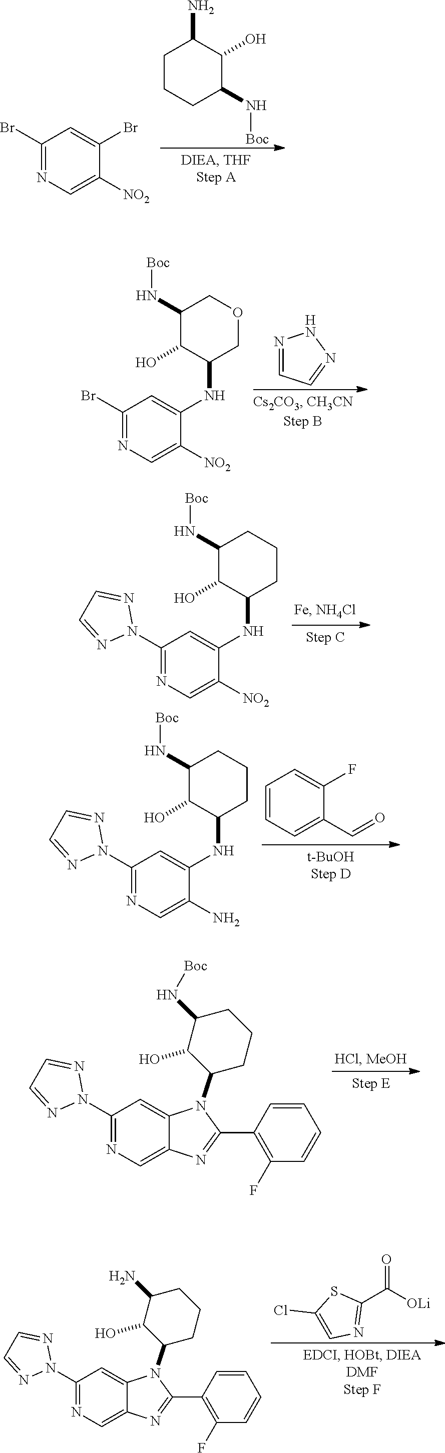

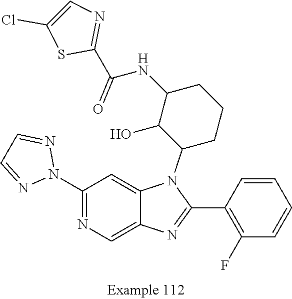 [SMILES: O=C(NC1CCCC(n2c(-c3ccccc3F)nc3cnc(-n4nccn4)cc32)C1O)c1ncc(Cl)s1]

### Step A. Synthesis of tert-butyl (3-((2-bromo-5-nitropyridin-4-yl)amino)-2-hydroxycyclohexyl)carbamate

To a solution of tert-butyl (3-amino-2-hydroxycyclohexyl)carbamate (500 mg, 2.17 mmol) (product of Step 1G in Intermediate Scheme 1 above) in THF (20 mL) were added DIEA (842 mg, 6.51 mmol) and 2,4-dibromo-5-nitro-pyridine (612 mg, 2.17 mmol). The reaction mixture was stirred at 70° C. for 15 hrs. The reaction mixture was concentrated to dryness. The residue was purified by silica gel column chromatography to give tert-butyl (3-((2-bromo-5-nitropyridin-4-yl)amino)-2-hydroxycyclohexyl)carbamate (157 mg, 16.8% yield). ¹H NMR (400 MHz, CDCl₃) δ 8.93 (s, 1H), 8.23 (br d, 1H), 7.16 (s, 1H), 4.64 (br s, 1H), 4.23 (br s, 1H), 3.60-3.48 (m, 2H), 3.38-3.33 (m, 1H), 2.09-2.00 (m, 3H), 1.91-1.84 (m, 1H), 1.45 (s, 9H), 1.39-1.30 (m, 2H); LC-MS: m/z 431.2 [M+H]⁺.

### Step B. Synthesis of tert-butyl (2-hydroxy-3-((5-nitro-2-(2H-1,2,3-triazol-2-yl)pyridin-4-yl)amino)cyclohexyl)carbamate

To a solution of tert-butyl (3-((2-bromo-5-nitropyridin-4-yl)amino)-2-hydroxycyclohexyl)carbamate (250 mg, 580 μmol) in MeCN (3 mL) were added Cs₂CO₃ (472 mg, 1.45 mmol) and 2H-triazole (48.0 mg, 696 μmol). The reaction mixture was stirred at 70° C. for 12 hrs, and then allowed to cool. The reaction mixture was quenched by the addition of H₂O (5 mL) at rt, diluted with H₂O (10 mL), and the aqueous portion extracted with EtOAc (10 mL×3). The combined organic layers were washed with brine (10 mL×3), dried over Na₂SO₄, filtered, and concentrated to dryness. The residue was purified by silica gel column chromatography to give tert-butyl (2-hydroxy-3-((5-nitro-2-(2H-1,2,3-triazol-2-yl)pyridin-4-yl)amino)cyclohexyl)carbamate (50 mg, 20.6% yield). LC-MS: m/z 420.2 [M+H]⁺.

### Step C. Synthesis of tert-butyl (3-((5-amino-2-(2H-1,2,3-triazol-2-yl)pyridin-4-yl)amino)-2-hydroxycyclohexyl)carbamate

To a solution of tert-butyl (2-hydroxy-3-((5-nitro-2-(2H-1,2,3-triazol-2-yl)pyridin-4-yl)amino)cyclohexyl)carbamate (35 mg, 83 μmol) in EtOH (0.5 mL) and H₂O (0.2 mL) were added Fe (23.3 mg, 417 μmol) and NH₄Cl (6.70 mg, 125 μmol). The reaction mixture was stirred at 80° C. for 1 hr. The reaction mixture was concentrated under reduced pressure to remove volatile solvents. The residue was diluted with THF (5 mL) and then filtered. The filter cake was washed with THF (20 mL×3). The combined organic layers were filtered again, and the filtrate concentrated to dryness to give tert-butyl (3-((5-amino-2-(2H-1,2,3-triazol-2-yl)pyridin-4-yl)amino)-2-hydroxycyclohexyl)carbamate (32 mg), which was used without further purification. LC-MS: m/z 390.3 [M+H]⁺.

### Step D. Synthesis of tert-butyl (3-(2-(2-fluorophenyl)-6-(2H-1,2,3-triazol-2-yl)-1H-imidazo[4,5-c]pyridin-1-yl)-2-hydroxycyclohexyl)carbamate

To a solution of tert-butyl (3-((5-amino-2-(2H-1,2,3-triazol-2-yl)pyridin-4-yl)amino)-2-hydroxycyclohexyl)carbamate (32 mg, 82 μmol) in t-BuOH (0.5 mL) was added 2-fluorobenzaldehyde (12.2 mg, 98.6 μmol). The reaction mixture was stirred at 85° C. for 20 hrs. The reaction mixture was concentrated to dryness. The residue was purified by silica gel column chromatography to give tert-butyl (3-(2-(2-fluorophenyl)-6-(2H-1,2,3-triazol-2-yl)-1H-imidazo[4,5-c]pyridin-1-yl)-2-hydroxycyclohexyl)carbamate (28 mg, 60.8% yield). LC-MS: m/z 494.2 [M+H]⁺.

### Step E. Synthesis of 2-amino-6-(2-(2-fluorophenyl)-6-(2H-1,2,3-triazol-2-yl)-1H-imidazo[4,5-c]pyridin-1-yl)cyclohexan-1-ol

To a solution of tert-butyl (3-(2-(2-fluorophenyl)-6-(2H-1,2,3-triazol-2-yl)-1H-imidazo[4,5-c]pyridin-1-yl)-2-hydroxycyclohexyl)carbamate (28.0 mg, 56.7 μmol) in MeOH (0.5 mL) was added HCl/MeOH (4 M, 1 mL). The reaction mixture was stirred at rt for 1 hr and then concentrated to dryness to give 2-amino-6-(2-(2-fluorophenyl)-6-(2H-1,2,3-triazol-2-yl)-1H-imidazo[4,5-c]pyridin-1-yl)cyclohexan-1-ol (23 mg), which was used without further purification. LC-MS: m/z 394.2 [M+H]⁺.

### Step F. Synthesis of 5-chloro-N-(3-(2-(2-fluorophenyl)-6-(2H-1,2,3-triazol-2-yl)-1H-imidazo[4,5-c]pyridin-1-yl)-2-hydroxycyclohexyl)thiazole-2-carboxamide

### Example 112

To a solution of 2-amino-6-(2-(2-fluorophenyl)-6-(2H-1,2,3-triazol-2-yl)-1H-imidazo[4,5-c]pyridin-1-yl)cyclohexan-1-ol (22.0 mg, 55.9 μmol) in DMF (1 mL) were added EDCI (16.1 mg, 83.9 μmol), HOBt (11.3 mg, 83.9 μmol), DIEA (21.7 mg, 168 μmol) and (5-chlorothiazole-2-carbonyl)oxylithium (9.48 mg, 55.9 μmol). The reaction mixture was stirred at rt for 12 hrs. The reaction mixture was quenched by the addition of H₂O (5 mL) at 15° C., diluted with H₂O (15 mL), and the aqueous portion extracted with DCM (20 mL×3). The combined organic layers were washed with brine (20 mL×3), dried over Na₂SO₄, filtered, and concentrated to dryness. The residue was purified by reversed-phase HPLC (column: Boston Green ODS 150*30 mm*5 um; mobile phase: [water(FA)-ACN]; B %: 40%-70%, 6 min) to give Example 112 (11.2 mg, 36.8% yield). ¹H NMR (400 MHz, CDCl₃) δ 8.51 (s, 1H), 7.95 (s, 2H), 7.92 (s, 1H), 7.86 (br t, 1H), 7.69 (br d, 1H), 7.64-7.58 (m, 1H), 7.57 (s, 1H), 7.42-7.38 (m, 1H), 7.30-7.25 (m, 1H), 5.28 (br s, 1H), 4.43-4.38 (m, 1H), 4.21-4.14 (m, 1H), 3.91 (br s, 1H), 2.36-2.28 (m, 3H), 1.98 (br s, 1H), 1.60-1.56 (m, 2H). LC-MS: m/z 539.2 [M+H]⁺.

### Preparation of Examples 138 and 139 Via General Scheme I (Methods A and D)

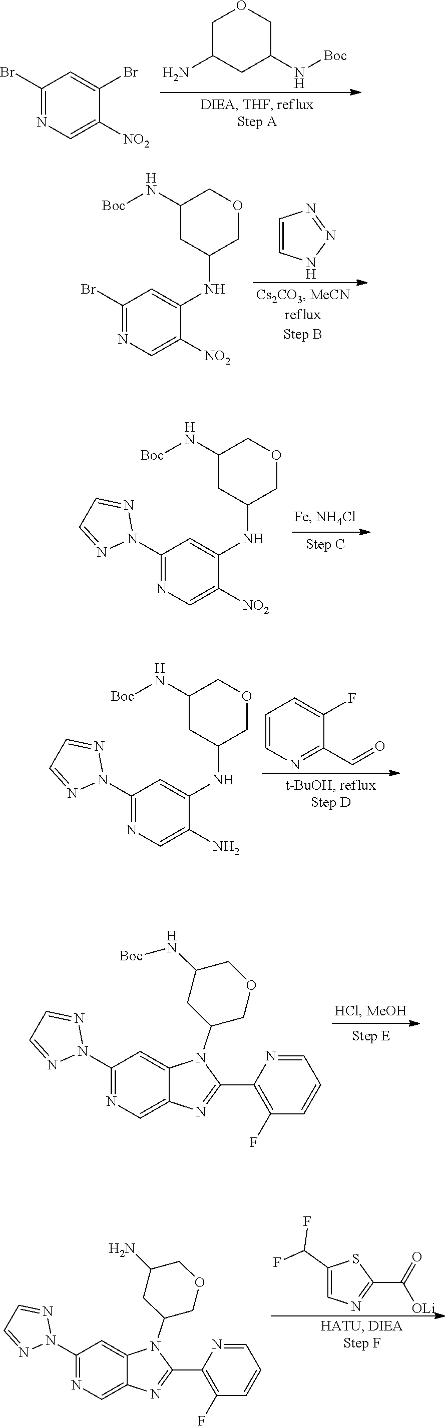

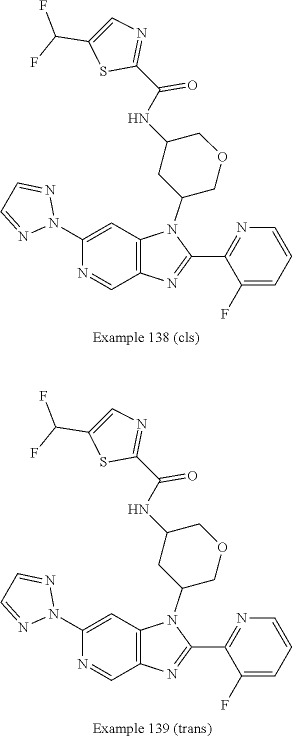 [SMILES: O=C(NC1COCC(n2c(-c3ncccc3F)nc3cnc(-n4nccn4)cc32)C1)c1ncc(C(F)F)s1 ; O=C(NC1COCC(n2c(-c3ncccc3F)nc3cnc(-n4nccn4)cc32)C1)c1ncc(C(F)F)s1]

### Step A. Synthesis of tert-butyl (5-((2-bromo-5-nitropyridin-4-yl)amino)tetrahydro-2H-pyran-3-yl)carbamate

To a solution of 2,4-dibromo-5-nitro-pyridine (652 mg, 2.31 mmol) and tert-butyl (5-aminotetrahydro-2H-pyran-3-yl)carbamate (500 mg, 2.31 mmol) in THF (10 mL) was added DIEA (896 mg, 6.94 mmol). The reaction mixture was stirred at 70° C. for 5 hrs, allowed to cool, and concentrated to dryness to give tert-butyl (5-((2-bromo-5-nitropyridin-4-yl)amino)tetrahydro-2H-pyran-3-yl)carbamate (1.0 g), which was used without further purification. LC-MS: m/z 416.9 [M+H]⁺.

### Step B. Synthesis of tert-butyl (5-((5-nitro-2-(2H-1,2,3-triazol-2-yl)pyridin-4-yl)amino)tetrahydro-2H-pyran-3-yl)carbamate

To a solution of tert-butyl (5-((2-bromo-5-nitropyridin-4-yl)amino)tetrahydro-2H-pyran-3-yl)carbamate (1.0 g, 2.4 mmol) and 1H-triazole (182 mg, 2.64 mmol) in MeCN (12 mL) was added Cs₂CO₃ (1.56 g, 4.79 mmol). The reaction mixture was stirred at 80° C. for 16 hrs, and then allowed to cool. The resulting slurry was filtered, and the filtrate was concentrated to dryness. The residue was purified by flash silica gel chromatography to give tert-butyl (5-((5-nitro-2-(2H-1,2,3-triazol-2-yl)pyridin-4-yl)amino)tetrahydro-2H-pyran-3-yl)carbamate (288 mg, 26.7% yield). LC-MS: m/z 406.7 [M+H]⁺.

### Step C. Synthesis of tert-butyl (5-((5-amino-2-(2H-1,2,3-triazol-2-yl)pyridin-4-yl)amino)tetrahydro-2H-pyran-3-yl)carbamate

To a solution of tert-butyl (5-((5-nitro-2-(2H-1,2,3-triazol-2-yl)pyridin-4-yl)amino)tetrahydro-2H-pyran-3-yl)carbamate (288 mg, 710 μmol) in THF (5 mL) were added Fe (198 mg, 3.55 mmol) and NH₄Cl (45.6 mg, 853 μmol) in H₂O (2.5 mL). The reaction mixture was stirred at 70° C. for 16 hrs, and then allowed to cool. The reaction mixture was filtered, and the filtrate was concentrated dryness to give tert-butyl (5-((5-amino-2-(2H-1,2,3-triazol-2-yl)pyridin-4-yl)amino)tetrahydro-2H-pyran-3-yl)carbamate (266 mg), which was used without further purification. LC-MS: m/z 375.9 [M+H]⁺.

### Step D. Synthesis of tert-butyl (5-(2-(3-fluoropyridin-2-yl)-6-(2H-1,2,3-triazol-2-yl)-1H-imidazo[4,5-c]pyridine-1-yl)tetrahydro-2H-pyran-3-yl)carbamate

To a solution of tert-butyl (5-((5-amino-2-(2H-1,2,3-triazol-2-yl)pyridine-4-yl)amino)tetrahydro-2H-pyran-3-yl)carbamate (266 mg) in t-BuOH (2.5 mL) was added 3-fluoropyridine-2-carbaldehyde (53.2 mg, 425 μmol). The reaction mixture was stirred at 90° C. for 16 hrs, and then allowed to cool. The reaction mixture was concentrated to dryness. The residue was purified by flash silica gel chromatography to give tert-butyl (5-(2-(3-fluoropyridin-2-yl)-6-(2H-1,2,3-triazol-2-yl)-1H-imidazo[4,5-c]pyridine-1-yl)tetrahydro-2H-pyran-3-yl)carbamate (90 mg, 42.3% yield). LC-MS: m/z 481.1 [M+H]⁺.

### Step E. Synthesis of 5-(2-(3-fluoropyridin-2-yl)-6-(2H-1,2,3-triazol-2-yl)-1H-imidazo[4,5-c]pyridin-1-yl)tetrahydro-2H-pyran-3-amine

A solution of tert-butyl (5-(2-(3-fluoropyridin-2-yl)-6-(2H-1,2,3-triazol-2-yl)-1H-imidazo[4,5-c]pyridin-1-yl)tetrahydro-2H-pyran-3-yl)carbamate (90 mg, 187 μmol) in HCl/dioxane (2 mL) was stirred at rt for 3 hrs. The reaction mixture was concentrated to dryness to give 5-(2-(3-fluoropyridin-2-yl)-6-(2H-1,2,3-triazol-2-yl)-1H-imidazo[4,5-c]pyridin-1-yl)tetrahydro-2H-pyran-3-amine (70 mg, HCl salt), which was used without further purification. LC-MS: m/z 381.0 [M+H]⁺.

### Step F. Synthesis of 5-(difluoromethyl)-N-(5-(2-(3-fluoropyridin-2-yl)-6-(2H-1,2,3-triazol-2-yl)-1H-imidazo[4,5-c]pyridin-1-yl)tetrahydro-2H-pyran-3-yl)thiazole-2-carboxamide (Examples 138 and 139)

To a solution of 5-(2-(3-fluoropyridin-2-yl)-6-(2H-1,2,3-triazol-2-yl)-1H-imidazo[4,5-c]pyridin-1-yl)tetrahydro-2H-pyran-3-amine (70 mg, 184 μmol) and lithium 5-(difluoromethyl)thiazole-2-carboxylate (39.6 mg, 220.8 μmol) in DMF (1 mL) were added HATU (91.0 mg, 239 μmol) and DIEA (119 mg, 920 μmol). The reaction mixture was stirred at rt for 16 hrs. The reaction mixture was purified by prep-HPLC (column: Welch Xtimate C18 150*30 mm*5 um; mobile phase: [water (0.05% NH₃H₂O+10 mM NH₄HCO₃)-ACN]; B %: 25%-55%, 9 min) to give Example 138 (relative stereochemistry at centers 3 and 5 of tetrahydropyranyl ring is cis) (13.5 mg, 13.1% yield) and Example 139 (relative stereochemistry at centers 3 and 5 of the tetrahydropyranyl ring is trans) (4.7 mg, 4.6% yield).
Example 138: ¹H NMR (400 MHz, CD₃OD) δ 8.99 (s, 1H), 8.72 (d, 1H), 8.63 (s, 1H), 8.13-8.10 (m, 3H), 7.98-7.93 (m, 1H), 7.79-7.74 (m, 1H), 7.17 (t, 1H), 5.10-5.06 (m, 1H), 4.28-4.19 (m, 3H), 4.07-4.04 (m, 1H), 3.60-3.54 (m, 1H), 2.85-2.79 (m, 1H), 2.53-2.50 (m, 1H). LC-MS: m/z 542.1 [M+H]⁺.
Example 139: ¹H NMR (400 MHz, CD₃OD) δ 8.99 (s, 1H), 8.62 (s, 1H), 8.37 (d, 1H), 8.22 (s, 1H), 8.09 (s, 2H), 7.83 (t, 1H), 7.60-7.56 (m, 1H), 7.24 (t, 1H), 5.27-5.24 (m, 1H), 4.42-4.35 (m, 2H), 4.21-4.18 (m, 1H), 4.02 (s, 2H), 2.82-2.76 (m, 1H), 2.63-2.60 (m, 1H). LC-MS: m/z 542.1 [M+H]⁺.

General Scheme I set forth above was used to synthesize the following compounds by using appropriate starting materials:

| Ex. | | | |
|---|---|---|---|
| No. | Structure | ¹H NMR | LC-MS and % ee |
| 4 | 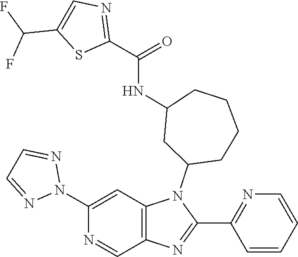 [SMILES: O=C(NC1CCCCC(n2c(-c3ccccn3)nc3cnc(-n4nccn4)cc32)C1)c1ncc(C(F)F)s1] | (400 MHz, CDCl₃) δ 9.08 (s, 1 H), 8.80 (d, 1 H), 8.37 (d, 1 H), 8.22 (s, 1 H), 7.96-7.92 (m, 4 H), 7.48-7.46 (m, 1 H), 7.28 (br s, 1 H), 6.88 (t, 1 H), 6.13 (br s, 1 H), 4.33 (br s, 1 H), 2.72 (q, 1 H), 2.53-2.46 (m, 2 H), 2.41-2.35 (m, 1 H), 2.26-2.21 (m, 1 H), 1.90 (br s, 3 H), 1.80-1.75 (m, 2 H) | m/z 536.3 [M + H]⁺ |
| | (relative stereochemistry at | | |
| | centers 1 and 3 of cyclohexyl | | |
| | ring is cis) | | |
| 12 | 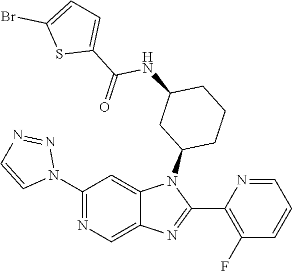 [SMILES: O=C(N[C@H]1CCC[C@@H](n2c(-c3ncccc3F)nc3cnc(-n4ccnn4)cc32)C1)c1ccc(Br)s1] | (400 MHz, CDCl₃) δ 8.96 (s, 1 H), 8.55-8.64 (m, 2 H), 8.37 (s, 1 H), 7.82 (d, 1 H), 7.69- 7.62 (m, 1 H), 7.50 (dt, 1 H), 7.18 (d, 1 H), 6.95 (d, 1 H), 6.01 (br d, 1 H), 4.82-4.72 (m, 1 H), 4.09-3.98 (m, 1 H), 2.44 (br d, 1 H), 2.29-2.21 (m, 1 H), 2.19-2.13 (m, 2 H), 2.10-2.04 (m, 1 H), 2.00-1.95 (m, 1 H), 1.46-1.31 (m, 2 H) | m/z 567.2 [M + H]⁺ |
| 13 | 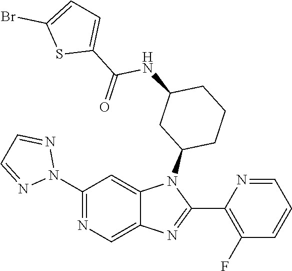 [SMILES: O=C(N[C@H]1CCC[C@@H](n2c(-c3ncccc3F)nc3cnc(-n4nccn4)cc32)C1)c1ccc(Br)s1] | (400 MHz, CDCl₃) δ 9.09 (s, 1 H), 8.67-8.62 (m, 1 H), 8.18 (s, 1 H), 7.98 (s, 2 H), 7.76-7.68 (m, 1 H), 7.58 (dt, 1 H), 7.34 (d, 1 H), 7.02 (d, 1 H), 6.39 (br d, 1 H), 4.89-4.80 (m, 1 H), 4.16-4.07 (m, 1 H), 2.42 (br d, 1 H), 2.27-2.19 (m, 4 H), 2.10- 2.04 (m, 1 H), 1.57-1.41 (m, 2 H) | m/z 567.1 [M + H]⁺ |
| 14 | 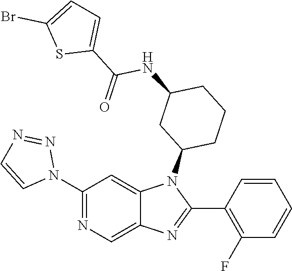 [SMILES: O=C(N[C@H]1CCC[C@@H](n2c(-c3ccccc3F)nc3cnc(-n4ccnn4)cc32)C1)c1ccc(Br)s1] | (400 MHz, CD₃OD) δ 8.95 (s, 1 H), 8.86 (d, 1 H), 8.52 (s, 1 H), 7.95 (d, 1 H), 7.80-7.67 (m, 2 H), 7.52-7.40 (m, 3 H), 7.13-7.07 (m, 1 H), 4.37-4.25 (m, 1 H), 4.01-3.88 (m, 1 H), 2.42 (q, 1 H), 2.35-2.22 (m, 2H), 2.15-1.96 (m, 3 H), 1.60- 1.41 (m, 2 H) | m/z 566.2 [M + H]⁺ |
| 15 | 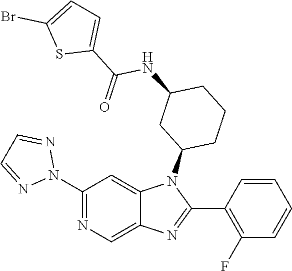 [SMILES: O=C(N[C@H]1CCC[C@@H](n2c(-c3ccccc3F)nc3cnc(-n4nccn4)cc32)C1)c1ccc(Br)s1] | (400 MHz, CD₃OD) δ 8.92 (s, 1 H), 8.44 (s, 1 H), 8.06 (s, 2 H), 7.79-7.67 (m, 2 H), 7.50- 7.39 (m, 3 H), 7.08 (d, 1 H), 4.31 (br t, 1 H), 3.93 (br t, 1 H), 2.41 (q, 1 H), 2.33-2.19 (m, 2 H), 2.15-1.96 (m, 3 H), 1.61-1.40 (m, 2 H) | m/z 566.2 [M + H]⁺ |
| 16 | 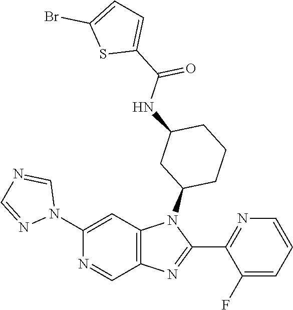 [SMILES: O=C(N[C@H]1CCC[C@@H](n2c(-c3ncccc3F)nc3cnc(-n4cncn4)cc32)C1)c1ccc(Br)s1] | (400 MHz, CD₃OD) δ 9.43 (s, 1 H), 8.97 (s, 1 H), 8.71 (d, 1 H), 8.35 (s, 1 H), 8.27 (s, 1 H), 7.95 (t, 1 H), 7.76 (dt, 1 H), 7.49 (d, 1 H), 7.12 (d, 1 H), 3.98 (br s, 1 H), 3.48 (s, 1 H), 2.39 (br d, 2 H), 2.29- 2.16 (m, 2 H), 2.04 (br d, 3 H), 1.55 (br s, 2 H) | m/z 567.2 [M + H]⁺ |
| 19 | 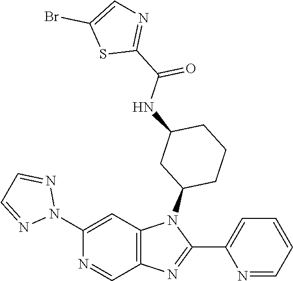 [SMILES: O=C(N[C@H]1CCC[C@@H](n2c(-c3ccccn3)nc3cnc(-n4nccn4)cc32)C1)c1ncc(Br)s1] | (400 MHz, CD₃OD) δ 8.94 (s, 1 H), 8.84 (d, 1 H), 8.50 (s, 1 H), 8.22 (d , 1 H), 8.11-8.03 (m, 3 H), 7.87 (s, 1 H), 7.65- 7.57 (m, 1 H), 5.68-5.56 (m, 1 H), 4.13-4.01 (m, 1 H), 2.57 (q, 1 H), 2.43-2.29 (m, 2 H), 2.19 (br d, 1 H), 2.12-2.04 (m, 2 H), 1.71-1.56 (m, 2 H) | m/z 550.2 [M + H]⁺ |
| 20 | 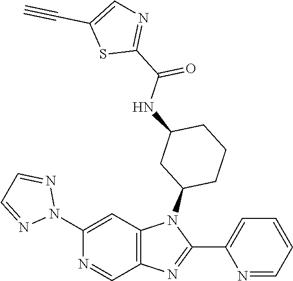 [SMILES: C#Cc1cnc(C(=O)N[C@H]2CCC[C@@H](n3c(-c4ccccn4)nc4cnc(-n5nccn5)cc43)C2)s1] | (400 MHz, CD₃OD) δ 8.95 (s, 1 H), 8.84 (d, 1 H), 8.51 (s, 1 H), 8.22 (d, 1 H), 8.10-8.04 (m, 3 H), 8.00 (s, 1 H), 7.61 (dd, 1 H), 5.62 (ddd, 1 H), 4.22 (s, 1 H), 4.15-4.02 (m, 1 H), 2.58 (q, 1 H), 2.43-2.30 (m, 2 H), 2.19 (br d, 1 H), 2.12-2.03 (m, 2 H), 1.72-1.56 (m, 2 H) | m/z 496.3 [M + H]⁺ |
| 21 | 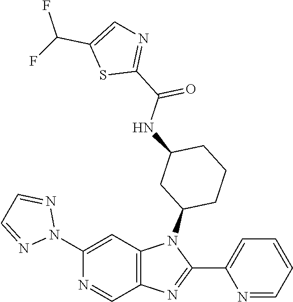 [SMILES: O=C(N[C@H]1CCC[C@@H](n2c(-c3ccccn3)nc3cnc(-n4nccn4)cc32)C1)c1ncc(C(F)F)s1] | (400 MHz, CD₃OD) δ 8.94 (s, 1 H), 8.85 (d, 1 H), 8.51 (s, 1 H), 8.23 (d, 1 H), 8.13 (s, 1 H), 8.10-8.05 (m, 3 H), 7.62 (dd, 1 H), 6.99-7.33 (m, 1 H), 5.71-5.56 (m, 1 H), 4.11 (br t, 1 H), 2.59 (q, 1 H), 2.47- 2.29 (m, 2 H), 2.20 (br d, 1 H), 2.08 (br d, 2 H), 1.73-1.57 (m, 2 H) | m/z 522.3 [M + H]⁺ |
| 22 | 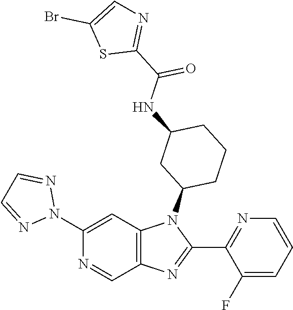 [SMILES: O=C(N[C@H]1CCC[C@@H](n2c(-c3ncccc3F)nc3cnc(-n4nccn4)cc32)C1)c1ncc(Br)s1] | (400 MHz, CD₃OD) δ 8.98 (s, 1 H), 8.74-8.68 (m, 1 H), 8.54 (s, 1 H), 8.09 (s, 2 H), 8.00- 7.91 (m, 1 H), 7.88 (s, 1 H), 7.76 (dt, 1 H), 4.85-4.77 (m, 1 H), 4.06-3.98 (m, 1 H), 2.52 (q, 1 H), 2.39 (br d, 1 H), 2.32- 2.23 (m, 1 H), 2.21-2.14 (m, 1 H), 2.07-2.00 (m, 2 H), 1.66- 1.50 (m, 2 H) | m/z 568.0 [M + H]⁺ |
| 23 | 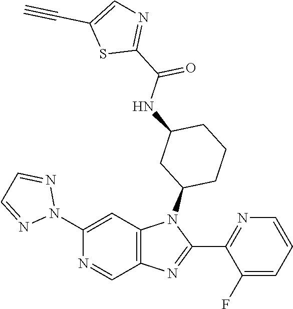 [SMILES: C#Cc1cnc(C(=O)N[C@H]2CCC[C@@H](n3c(-c4ncccc4F)nc4cnc(-n5nccn5)cc43)C2)s1] | (400 MHz, CD₃OD) δ 8.97 (s, 1 H), 8.70 (d, 1 H), 8.52 (s, 1 H), 8.07 (s, 2 H), 7.98 (s, 1 H), 7.94 (t , 1 H), 7.74 (dt, 1 H), 4.84-4.76 (m, 1 H), 4.21 (s, 1 H), 4.06-3.98 (m, 1 H), 2.52 (q, 1 H), 2.42-2.35 (m, 1 H), 2.31-2.22 (m, 1 H), 2.19- 2.12 (m, 1 H), 2.06-1.99 (m, 2 H), 1.66-1.50 (m, 2 H) | m/z 514.1 [M + H]⁺ |
| 24 | 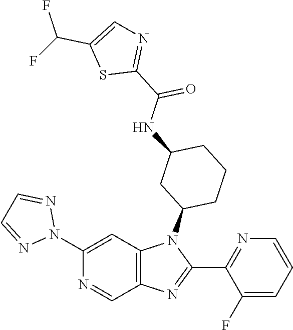 [SMILES: O=C(N[C@H]1CCC[C@@H](n2c(-c3ncccc3F)nc3cnc(-n4nccn4)cc32)C1)c1ncc(C(F)F)s1] | (400 MHz, CD₃OD) δ 8.99 (s, 1 H), 8.73-8.69 (m, 1 H), 8.54 (s, 1 H), 8.14 (t, 1 H), 8.09 (s, 2 H), 8.00-7.91 (m, 1 H), 7.76 (dt, 1 H), 7.34-7.01 (m, 1 H), 4.85-4.76 (m, 1 H), 4.10-4.01 (m, 1 H), 2.54 (q, 1 H), 2.46- 2.38 (m, 1 H), 2.33-2.24 (m, 1 H), 2.22-2.13 (m, 1 H), 2.08- 2.01 (m, 2 H), 1.69-1.52 (m, 2 H) | m/z 540.2 [M + H]⁺ |
| 26 | 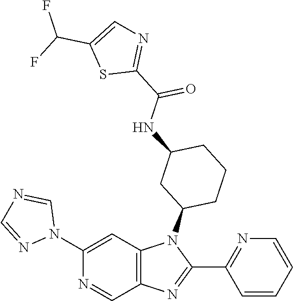 [SMILES: O=C(N[C@H]1CCC[C@@H](n2c(-c3ccccn3)nc3cnc(-n4cncn4)cc32)C1)c1ncc(C(F)F)s1] | (400 MHz, CDCl₃) δ 9.28 (s, 1 H), 8.97 (s, 1 H), 8.80 (d, 1 H), 8.32 (d, 1 H), 8.16 (d, 2 H), 8.01-7.92 (m, 2 H), 7.53-7.46 (m, 1 H), 7.32 (br d, 1 H), 7.08-6.76 (m, 1 H), 5.86 (br s, 1 H), 4.21 (br d, 1 H), 2.57 (br d, 1 H), 2.41-2.21 (m, 4 H, 2.16-2.06 (m, 1 H), 1.76-1.66 (m, 1 H), 1.57 (br dd, 1 H) | m/z 522.2 [M + H]⁺ |
| 27 | 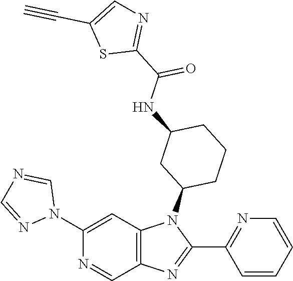 [SMILES: C#Cc1cnc(C(=O)N[C@H]2CCC[C@@H](n3c(-c4ccccn4)nc4cnc(-n5cncn5)cc43)C2)s1] | (400 MHz, CDCl₃) δ 9.27 (s, 1 H), 8.97 (s, 1 H), 8.79 (d, 1 H), 8.32 (d, 1 H), 8.16 (d, 2 H), 8.00-7.92 (m, 2 H), 7.49 (dd, 1 H), 7.26 (br s, 1 H), 5.85 (br t, 1 H), 4.28- 4.12 (m, 1 H), 3.57 (s, 1 H), 2.54 (br s, 1 H), 2.38-2.10 (m, 5 H), 1.68 (br d, 1 H), 1.56-1.49 (m, 1 H) | m/z 496.1 [M + H]⁺ |
| 28 | 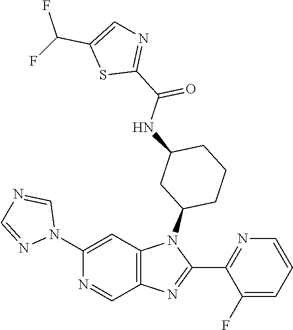 [SMILES: O=C(N[C@H]1CCC[C@@H](n2c(-c3ncccc3F)nc3cnc(-n4cncn4)cc32)C1)c1ncc(C(F)F)s1] | (400 MHz, CDCl₃) δ 9.29 (s, 1 H), 9.03 (s, 1 H), 8.68 (d, 1 H), 8.17 (d, 2 H), 8.00 (s, 1 H), 7.79-7.72 (m, 1 H), 7.60 (dt, 1 H), 7.31 (br d, 1 H), 7.09-6.78 (m, 1 H), 4.93-4.83 (m, 1 H), 4.19-4.06 (m, 1 H), 2.61-2.52 (m, 1 H), 2.38-2.08 (m, 5 H), 1.58-1.51 (m, 2 H) | m/z 540.1 [M + H]⁺ |
| 29 | 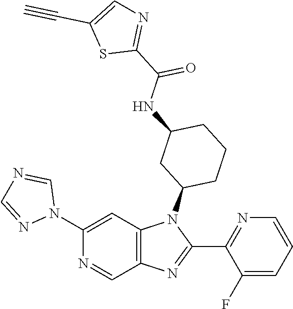 [SMILES: C#Cc1cnc(C(=O)N[C@H]2CCC[C@@H](n3c(-c4ncccc4F)nc4cnc(-n5cncn5)cc43)C2)s1] | (400 MHz, CDCl₃) δ 9.29 (s, 1 H), 9.02 (s, 1 H), 8.67 (d, 1 H), 8.17 (d, 2 H), 7.93 (s, 1 H), 7.75 (t, 1 H), 7.60 (dt, 1 H), 7.26 (s, 1 H), 4.86 (br d, 1 H), 4.17-4.04 (m, 1 H), 3.57 (s, 1 H), 2.56 (br d, 1 H), 2.36-2.06 (m, 5 H), 1.59-1.47 (m, 2 H) | m/z 514.2 [M + H]⁺ |
| 30 | 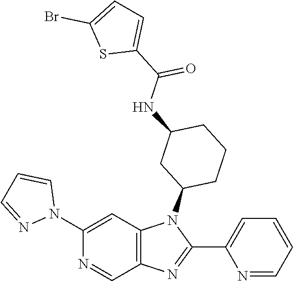 [SMILES: O=C(N[C@H]1CCC[C@@H](n2c(-c3ccccn3)nc3cnc(-n4cccn4)cc32)C1)c1ccc(Br)s1] | (400 MHz, CD₃OD) δ 8.86 (s, 1 H), 8.83 (d, 1 H), 8.69 (d, 1 H), 8.32 (s, 1 H), 8.18 (d, 1 H), 8.06 (td, 1 H), 7.88 (s, 1 H), 7.84 (d, 1 H), 7.62-7.58 (m, 1 H), 6.59-6.57 (m, 1 H), 5.59-5.53 (m, 1 H), 4.11-4.01 (m, 1 H), 2.56 (d, 1 H), 2.41- 2.34 (m, 2 H), 2.16 (br d, 1 H), 2.06 (br s, 1 H), 1.70-1.55 (m, 2 H), 1.35-1.26 (m, 1 H) | m/z 549.1 [M + H]⁺ |
| 32 | 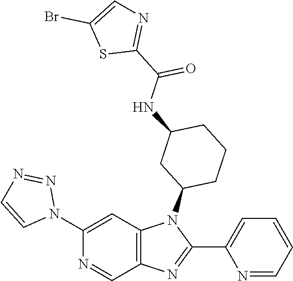 [SMILES: O=C(N[C@H]1CCC[C@@H](n2c(-c3ccccn3)nc3cnc(-n4ccnn4)cc32)C1)c1ncc(Br)s1] | (400 MHz, DMSO-d₆) δ 9.10- 9.02 (m, 2 H), 8.91 (s, 1 H), 8.84 (br d, 1 H), 8.46 (s, 1 H), 8.24 (br d, 1 H), 8.13-8.07 (m, 2 H), 8.03 (s, 1 H), 7.67-7.61 (m, 1 H), 5.70-5.55 (m, 1 H), 4.01 (br d, 1 H), 2.67 (q, 1 H), 2.22-2.12 (m, 2 H), 2.07 (br s, 1 H), 1.94 (br d, 1 H), 1.85 (br d, 1 H), 1.72-1.61 (m, 1 H), 1.49 (br d, 1 H) | m/z 552.2 [M + H]⁺ |
| 33 | 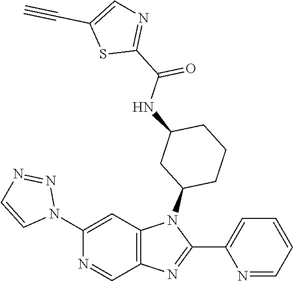 [SMILES: C#Cc1cnc(C(=O)N[C@H]2CCC[C@@H](n3c(-c4ccccn4)nc4cnc(-n5ccnn5)cc43)C2)s1] | (400 MHz, CD₃OD) δ 8.96 (s, 1 H), 8.87-8.82 (m, 2 H), 8.56 (s, 1 H), 8.21 (d, 1 H), 8.07 (t, 1 H), 8.00 (s, 1 H), 7.96 (s, 1 H), 7.61 (dd, 1 H), 5.61 (br t, 1 H), 4.62 (br s, 1 H), 4.09 (br t, 1 H), 2.58 (q, 1 H), 2.44-2.33 (m, 2 H), 2.18 (br d, 1 H), 2.10-2.04 (m, 2 H), 1.74-1.58 (m, 2 H) | m/z 496.2 [M + H]⁺ |
| 34 | 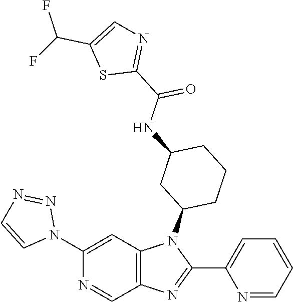 [SMILES: O=C(N[C@H]1CCC[C@@H](n2c(-c3ccccn3)nc3cnc(-n4ccnn4)cc32)C1)c1ncc(C(F)F)s1] | (400 MHz, DMSO-d₆) δ 9.20 (br d, 1 H), 9.06 (s, 1 H), 8.91 (s, 1 H), 8.84 (br d, 1 H), 8.47 (s, 1 H), 8.31 (s, 1 H), 8.25 (br d, 1 H), 8.10 (br t, 1 H), 8.03 (s, 1 H), 7.68-7.62 (m, 1 H), 7.60-7.49 (m, 1 H), 5.70-5.56 (m, 1 H), 4.04 (br d, 1 H), 2.69 (q, 1 H), 2.23-2.13 (m, 2 H), 2.07 (br s, 1 H), 1.95 (br d, 1 H), 1.87 (br d, 1 H), 1.76- 1.63 (m, 1 H), 1.57-1.45 (m, 1 H) | m/z 522.2 [M + H]⁺ |
| 35 | 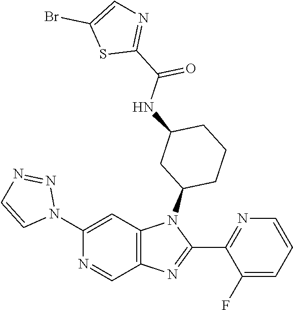 [SMILES: O=C(N[C@H]1CCC[C@@H](n2c(-c3ncccc3F)nc3cnc(-n4ccnn4)cc32)C1)c1ncc(Br)s1] | (400 MHz, CD₃OD) δ 8.99 (s, 1 H), 8.87 (d, 1 H), 8.70 (d, 1 H), 8.58 (s, 1 H), 7.99-7.91 (m, 2 H), 7.87 (s, 1 H), 7.75 (dt, 1H), 4.80 (tt, 1 H), 4.01 (ddd, 1 H), 2.52 (q, 1 H), 2.38 (br d, 1 H), 2.32-2.22 (m, 1 H), 2.20-2.12 (m, 1 H), 2.08-1.96 (m, 2 H), 1.68-1.48 (m, 2 H) | m/z 568.2 [M + H]⁺ |
| 36 | 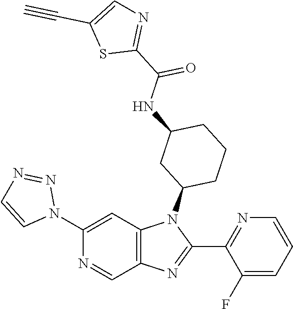 [SMILES: C#Cc1cnc(C(=O)N[C@H]2CCC[C@@H](n3c(-c4ncccc4F)nc4cnc(-n5ccnn5)cc43)C2)s1] | (400 MHz, CD3OD) 8 8.99 (s, 1 H), 8.87 (s, 1 H), 8.70 (d, 1 H), 8.58 (s, 1 H), 7.99 (s, 1 H), 7.98-7.90 (m, 2 H), 7.75 (dt, , 1 H), 4.80 (ddd, 1 H), 4.22 (s, 1 H), 4.08-3.97 (m, 1 H), 2.52 (q, 1 H), 2.38 (br d, 1 H), 2.32-2.23 (m, 1 H), 2.19-2.12 (m, 1 H), 2.07-1.97 (m, 2 H), 1.68- 1.49 (m, 2 H) | m/z 514.3 [M + H]⁺ |
| 37 | 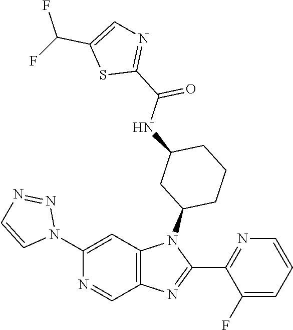 [SMILES: O=C(N[C@H]1CCC[C@@H](n2c(-c3ncccc3F)nc3cnc(-n4ccnn4)cc32)C1)c1ncc(C(F)F)s1] | (400 MHz, CD₃OD) δ 8.99 (s, 1 H), 8.86 (d, 1 H), 8.70 (d, 1 H), 8.62-8.56 (m, 1 H), 8.13 (s, 1 H), 7.98-7.91 (m, 2 H), 7.75 (dt, 1 H), 7.33-6.99 (m, 1 H), 4.82 (qd , 1 H), 4.10- 3.99 (m, 1 H), 2.54 (q, 1 H), 2.40 (br d, 1 H), 2.32-2.23 (m, 1 H), 2.20-2.13 (m, 1 H), 2.08- 2.00 (m, 2 H), 1.69-1.50 (m, 2 H) | m/z 540.3 [M + H]⁺ |
| 38 | 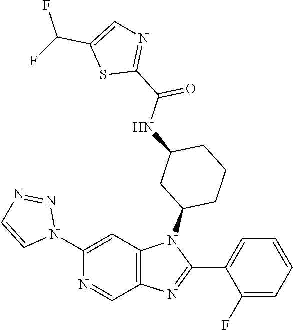 [SMILES: O=C(N[C@H]1CCC[C@@H](n2c(-c3ccccc3F)nc3cnc(-n4ccnn4)cc32)C1)c1ncc(C(F)F)s1] | (400 MHz, CD₃OD) δ 8.96 (s, 1 H), 8.86 (d, 1 H), 8.56 (s, 1 H), 8.15-8.11 (m, 1 H), 7.96 (d, 1 H), 7.78-7.70 (m, 2 H), 7.51-7.42 (m, 2 H), 7.30-7.01 (m, 1 H), 4.34 (br t, 1 H), 4.07- 3.96 (m, 1 H), 2.55 (q, 1 H), 2.35-2.26 (m, 2 H), 2.09-1.99 (m, 3 H), 1.71-1.60 (m, 1 H), 1.57-1.46 (m, 1 H) | m/z 539.3 [M + H]⁺ |
| 39 | 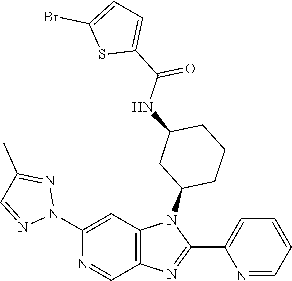 [SMILES: Cc1cnn(-c2cc3c(cn2)nc(-c2ccccn2)n3[C@@H]2CCC[C@H](NC(=O)c3ccc(Br)s3)C2)n1] | (400 MHz, CD₃OD) δ 8.91 (s, 1 H), 8.83 (d, 1 H), 8.40 (s, 1 H), 8.21 (d, 1 H), 8.06 (td, 1 H), 7.84 (s, 1 H), 7.61 (dd, 1 H), 7.49 (d, 1 H), 7.11 (d, 1 H), 5.67-5.54 (m, 1 H), 4.11-3.98 (m, 1 H), 2.51- 2.45 (m, 4 H), 2.41-2.28 (m, 2 H), 2.18 (br d, 1 H), 2.09- 2.04 (m, 2 H), 1.63-1.54 (m, 2 H) | m/z 563.0 [M + H]⁺ |
| 40 | 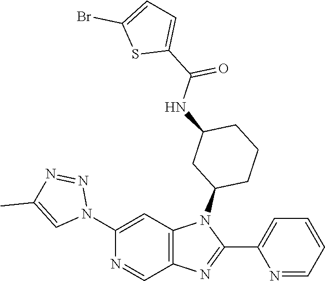 [SMILES: Cc1cn(-c2cc3c(cn2)nc(-c2ccccn2)n3[C@@H]2CCC[C@H](NC(=O)c3ccc(Br)s3)C2)nn1] | (400 MHz, CD₃OD) δ 8.96 (s, 1 H), 8.85 (d, 1 H), 8.61 (s, 1 H), 8.51 (s, 1 H), 8.22 (d, 1 H), 8.11-8.06 (m, 1 H), 7.64-7.60 (m, 1 H), 7.50 (d, 1 H), 7.13 (d, 1 H), 5.61 (br s, 1 H), 4.05 (br s, 1 H), 2.46 (s, 4 H), 2.42-2.33 (m, 2 H), 2.19 (br d, 1 H), 2.08 (br d, 2 H), 1.60 (br t, 2 H) | m/z 563.1 [M + H]⁺ |
| 49 | 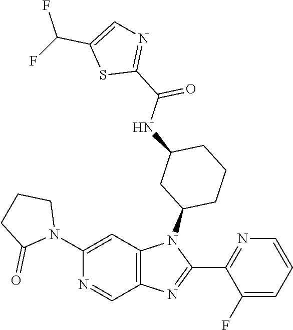 [SMILES: O=C(N[C@H]1CCC[C@@H](n2c(-c3ncccc3F)nc3cnc(N4CCCC4=O)cc32)C1)c1ncc(C(F)F)s1] | (400 MHz, CDCl₃) δ 9.01-8.84 (m, 2 H), 8.65 (br d, 1 H), 7.98 (s, 1 H), 7.76-7.49 (m, 2 H), 7.32 (br d, 1 H), 7.10-6.75 (m, 1 H), 4.85-4.73 (m, 1 H), 4.28 (br t, 2 H), 4.14-4.03 (m, 1 H), 2.76 (br t, 2 H), 2.51-2.03 (m, 8 H), 1.58-1.46 (m, 2 H) | m/z 556.2 [M + H]⁺ |
| 58 | 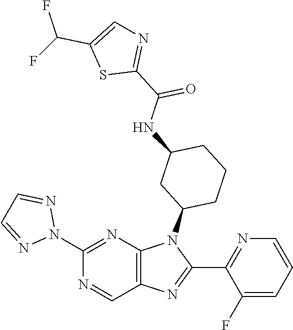 [SMILES: O=C(N[C@H]1CCC[C@@H](n2c(-c3ncccc3F)nc3cnc(-n4nccn4)nc32)C1)c1ncc(C(F)F)s1] | (400 MHz, CDCl₃) δ 9.28 (s, 1 H), 8.68 (d, 1 H), 8.03 (s, 2 H), 7.97 (t, 1 H), 7.78-7.70 (m, 1 H), 7.61 (dt, 1 H), 7.33 (br d, 1 H), 7.08-6.73 (m, 1 H), 4.93-4.82 (m, 1 H), 4.16-4.04 (m, 1 H), 2.95-2.72 (m, 2 H), 2.47 (br d, 1 H), 2.21-2.05 (m, 3 H), 1.62-1.47 (m, 2 H) | m/z 541.1 [M + H]⁺ |
| 59 | 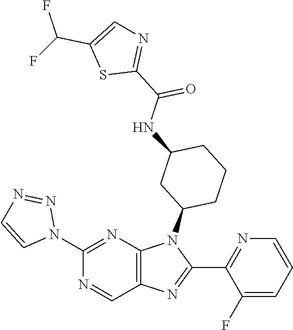 [SMILES: O=C(N[C@H]1CCC[C@@H](n2c(-c3ncccc3F)nc3cnc(-n4ccnn4)nc32)C1)c1ncc(C(F)F)s1] | (400 MHz, CDCl₃) δ 9.23 (s, 1 H), 8.71-8.68 (m, 2 H), 7.97 (s, 1 H), 7.91 (d, 1 H), 7.79-7.73 (m, 1 H), 7.62 (dt, 1 H), 7.35 (br d, 1 H), 7.06- 6.76 (m, 1 H), 4.94-4.84 (m, 1 H), 4.15-4.04 (m, 1 H), 2.94-2.74 (m, 2 H), 2.48 (br d, 1 H), 2.22-2.07 (m, 3 H), 1.61-1.51 (m, 2 H) | m/z 541.1 [M + H]⁺ |
| 60 | 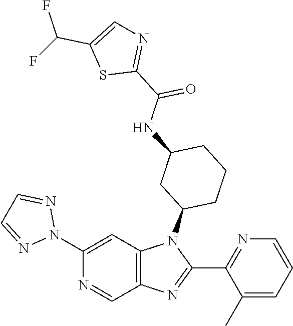 [SMILES: Cc1cccnc1-c1nc2cnc(-n3nccn3)cc2n1[C@@H]1CCC[C@H](NC(=O)c2ncc(C(F)F)s2)C1] | (400 MHz, DMSO-d₆) δ 9.16 (br d, 1 H), 9.02 (s, 1 H), 8.65 (d, 1 H), 8.36 (s, 1 H), 8.32 (s, 1 H), 8.21 (s, 2 H), 7.95 (d, 1 H), 7.57 (dd, 1 H), 7.49-7.29 (m, 1 H), 4.53 (br s, 1 H), 3.94 (br d, 1 H), 2.41 (s, 3 H), 2.35- 2.07 (m, 2 H), 2.00 (br s, 2 H), 1.90-1.75 (m, 2 H), 1.62-1.36 (m, 2 H) | m/z 536.2 [M + H]⁺ |
| 61 | 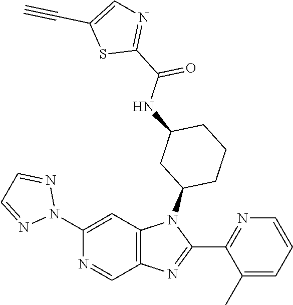 [SMILES: C#Cc1cnc(C(=O)N[C@H]2CCC[C@@H](n3c(-c4ncccc4C)nc4cnc(-n5nccn5)cc43)C2)s1] | (400 MHz, DMSO-d₆) δ 9.07- 8.96 (m, 2 H), 8.60 (br s, 1 H), 8.31 (br s, 1 H), 8.17 (br s, 3 H), 7.90 (br s, 1 H), 7.53 (br s, 1 H), 4.92 (br s, 1 H), 4.49 (br s, 1 H), 3.87 (br s, 1 H), 2.36 (br s, 3 H), 2.14- 1.88 (m, 4 H), 1.85-1.69 (m, 2 H), 1.54-1.32 (m, 2 H) | m/z 510.2 [M + H]⁺ |
| 62 | 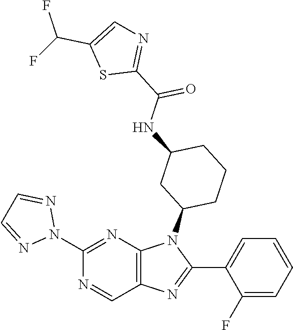 [SMILES: O=C(N[C@H]1CCC[C@@H](n2c(-c3ccccc3F)nc3cnc(-n4nccn4)nc32)C1)c1ncc(C(F)F)s1] | (400 MHz, CDCl₃) δ 9.21 (s, 1 H), 8.01 (s, 2 H), 7.96 (s, 1 H), 7.70-7.63 (m, 2 H), 7.45-7.40 (m, 1 H), 7.36- 7.29 (m, 2 H), 7.06-6.75 (m, 1 H), 4.24 (br t, 1 H), 4.11-4.00 (m, 1 H), 2.93- 2.75 (m, 2 H), 2.33 (br d, 1 H), 2.14-2.00 (m, 3 H), 1.58 (br d, 2 H) | m/z 540.2 [M + H]⁺ |
| 64 | 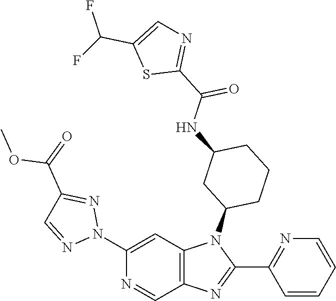 [SMILES: COC(=O)c1cnn(-c2cc3c(cn2)nc(-c2ccccn2)n3[C@@H]2CCC[C@H](NC(=O)c3ncc(C(F)F)s3)C2)n1] | (400 MHz, CD₃OD) δ 8.96 (s, 1 H), 8.85 (d, 1 H), 8.58 (s, 1 H), 8.48 (s, 1 H), 8.23 (d, 1 H), 8.13 (s, 1 H), 8.09-8.05 (m, 1 H), 7.87 (d, 1 H), 7.73 (d, 1 H), 7.62 (dd, 1 H), 7.57- 7.52 (m, 1 H), 7.51-7.46 (m, 1 H), 7.30-7.01 (m, 1 H), 5.71-5.59 (m, 1 H), 4.15-4.08 (m, 1 H), 4.02 (s, 3 H), 2.59 (q, 1 H), 2.47-2.35 (m, 2 H), 2.22-2.16 (m, 1 H), 2.08 (br d, 2 H), 1.74-1.60 (m, 2 H) | m/z 580.2 [M + H]⁺ |
| 65 | 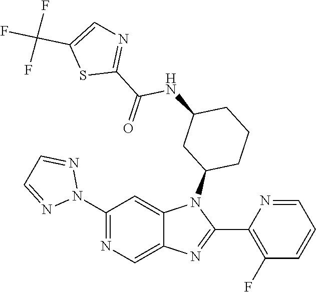 [SMILES: O=C(N[C@H]1CCC[C@@H](n2c(-c3ncccc3F)nc3cnc(-n4nccn4)cc32)C1)c1ncc(C(F)(F)F)s1] | (400 MHz, CD₃OD) δ 8.96 (br s, 1 H), 8.68 (d, 1 H), 8.51 (s, 1 H), 8.31 (s, 1 H), 8.06 (s, 2 H), 7.92 (t, 1 H), 7.73 (dt, 1 H), 4.59 (s, 1 H), 4.09-3.98 (m, 1 H), 2.57-2.37 (m, 2 H), 2.33-2.13 (m, 2 H), 2.02 (br d, 2 H), 1.67-1.50 (m, 2 H) | m/z 558.2 [M + H]⁺ |
| 66 | 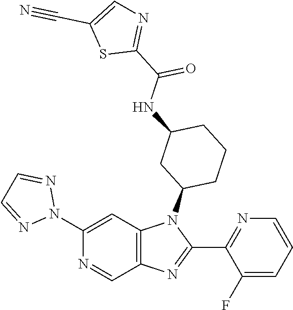 [SMILES: N#Cc1cnc(C(=O)N[C@H]2CCC[C@@H](n3c(-c4ncccc4F)nc4cnc(-n5nccn5)cc43)C2)s1] | (400 MHz, CD₃OD) δ 8.96 (s, 1 H), 8.69 (d, 1 H), 8.52 (s, 1 H), 8.47 (s, 1 H), 8.07 (s, 2 H), 7.97-7.89 (m, 1 H), 7.74 (dt, 1 H), 4.79 (br s, 1 H), 4.03 (br s, 1 H), 2.54-2.47 (m, 1 H), 2.39 (br s, 1 H), 2.26 (br t, 1 H), 2.15 (br d, 1 H), 2.02 (br s, 2 H), 1.65-1.51 (m, 2 H) | m/z 515.2 [M + H]⁺ |
| 70 | 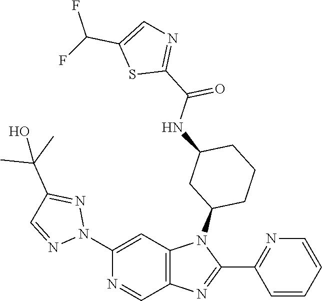 [SMILES: CC(C)(O)c1cnn(-c2cc3c(cn2)nc(-c2ccccn2)n3[C@@H]2CCC[C@H](NC(=O)c3ncc(C(F)F)s3)C2)n1] | (400 MHz, CD₃OD) δ 8.94 (s, 1 H), 8.85 (d, 1 H), 8.46 (s, 1 H), 8.23-8.07 (m, 3 H), 8.00 (s, 1 H), 7.64-7.61 (m, 1 H), 7.16 (t, 1 H), 5.64-5.58 (m, 1 H), 4.13-4.10 (m, 1 H), 2.62-2.55 (m, 1 H), 2.44-2.36 (m, 2 H), 2.21-2.17 (m, 1 H), 2.10-2.07 (m, 2 H), 1.71-1.63 (m, 1 H) | m/z 580.3 [M + H]⁺ |
| 92 | 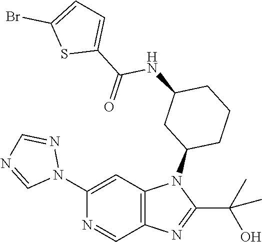 [SMILES: CC(C)(O)c1nc2cnc(-n3cncn3)cc2n1[C@@H]1CCC[C@H](NC(=O)c2ccc(Br)s2)C1] | (400 MHz, CDCl₃) δ 9.19 (s, 1 H), 8.82 (s, 1 H), 8.13-7.91 (m, 2 H), 7.18 (d, 1 H), 6.98 (d, 1 H), 6.04 (br d, 1 H), 5.36 (br s, 1 H), 4.15 (br dd, 1 H), 2.88 (s, 1 H), 2.45-2.37 (m, 1 H), 2.29-1.99 (m, 5 H), 1.82 (d, 6 H), 1.60-1.54 (m, 2 H) | m/z 532.4 [M + H]⁺ |
| 93 | 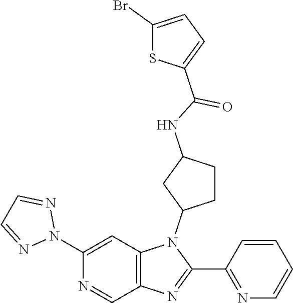 [SMILES: O=C(NC1CCC(n2c(-c3ccccn3)nc3cnc(-n4nccn4)cc32)C1)c1ccc(Br)s1] | (400 MHz, DMSO-d₆) δ 9.06 (s, 1 H), 8.86-8.78 (m, 2 H), 8.32 (s, 1 H), 8.26 (d, 1 H), 8.17 (s, 2 H), 8.10 (td, 1 H), 7.68 (d, 1 H), 7.64 (dd, 1 H), 7.34 (d, 1 H), 6.05 (q, 1 H), 4.45-4.23 (m, 1 H), 2.42-2.06 (m, 6 H) | m/z 537.0 [M + H]⁺ |
| | (relative stereochemistry at | | |
| | centers 1 and 3 of cyclopentyl | | |
| | ring is cis) | | |
| 94 | 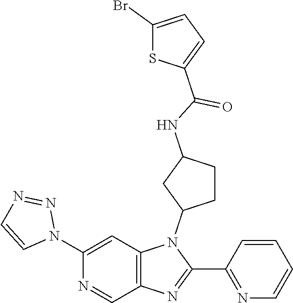 [SMILES: O=C(NC1CCC(n2c(-c3ccccn3)nc3cnc(-n4ccnn4)cc32)C1)c1ccc(Br)s1] | (400 MHz, DMSO-d₆) δ 9.09 (s, 1 H), 8.93 (s, 1 H), 8.86- 8.83 (m, 2 H), 8.44 (s, 1 H), 8.26 (d, 1 H), 8.10 (td, 1 H), 8.04 (s, 1 H), 7.68 (d, 1 H), 7.67-7.65 (m, 1 H), 7.30 (d, 1 H), 6.12-6.00 (m, 1 H), 4.41- 4.33 (m, 1 H), 2.41-2.25 (m, 3 H), 2.25-2.04 (m, 3 H) | m/z 535.0 [M + H]⁺ |
| | (relative stereochemistry at | | |
| | centers 1 and 3 of cyclopentyl | | |
| | ring is cis) | | |
| 96 | 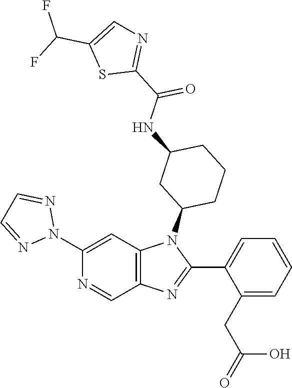 [SMILES: O=C(O)Cc1ccccc1-c1nc2cnc(-n3nccn3)cc2n1[C@@H]1CCC[C@H](NC(=O)c2ncc(C(F)F)s2)C1] | (400 MHz, CD₃OD) δ 8.88 (s, 1 H), 8.46 (s, 1 H), 8.11 (s, 1 H), 8.05 (s, 2 H), 7.63-7.49 (m, 4 H), 7.31-6.97 (m, 1 H), 4.56 (s, 1 H), 4.40-4.28 (m, 1 H), 3.99-3.80 (m, 2 H), 2.55- 2.38 (m, 2 H), 2.30-2.14 (m, 2 H), 2.00 (br d, 2 H), 1.67- 1.42 (m, 2 H) | m/z 579.3 [M + H]⁺ |
| 106 | 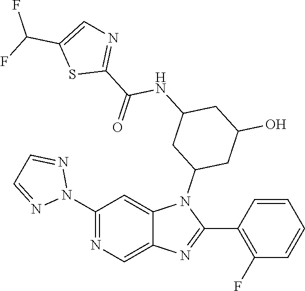 [SMILES: O=C(NC1CC(O)CC(n2c(-c3ccccc3F)nc3cnc(-n4nccn4)cc32)C1)c1ncc(C(F)F)s1] | (400 MHz, CD₃OD) δ 9.13 (d, 1 H), 8.44-8.32 (m, 4 H), 7.76- 7.75 (m, 2 H), 7.54-7.28 (m, 4 H), 4.88 (s, 1 H), 4.61 (br s, 1 H), 4.27 (br s, 1 H), 4.19 (s, 1 H), 2.66-2.55 (m, 1 H), 2.34- 2.26 (m, 1 H), 2.11-2.09 (m, 1 H), 1.94-1.90 (m, 1 H), 1.84- 1.82 (m, 2 H) | m/z 555.2 [M + H]⁺ |
| 107 | 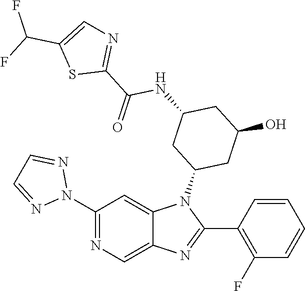 [SMILES: O=C(N[C@@H]1C[C@@H](O)C[C@H](n2c(-c3ccccc3F)nc3cnc(-n4nccn4)cc32)C1)c1ncc(C(F)F)s1] | (400 MHz, CD₃OD) δ 8.95 (s, 1 H), 8.49 (s, 1 H), 8.14 (s, 1 H), 8.08 (s, 2 H), 7.77-7.69 (m, 2 H), 7.50-7.42 (m, 2 H), 7.31-7.01 (m, 1 H), 4.83 (br s, 1 H), 4.46 (br t, 1 H), 4.37 (br s, 1 H), 2.59-2.47 (m, 2 H), 2.34 (br d, 1 H), 2.10 (br d, 2 H), 1.93-1.86 (m, 1 H) | m/z 555.2 [M + H]⁺; column: DAICEL CHIRALCEL OD-H (250 mm*30 mm, 5 um); mobile phase: [0.1% NH₃H₂O EtOH]; B %: 30%- 30%, min; Rt = 3.383 min 98.2% ee |
| 108 | 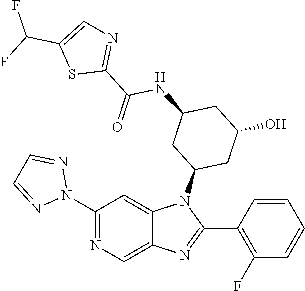 [SMILES: O=C(N[C@H]1C[C@H](O)C[C@@H](n2c(-c3ccccc3F)nc3cnc(-n4nccn4)cc32)C1)c1ncc(C(F)F)s1] | (400 MHz, CD₃OD) δ 8.94 (s, 1 H), 8.48 (s, 1 H), 8.13 (s, 1 H), 8.08 (s, 2 H), 7.77-7.69 (m, 2 H), 7.50-7.41 (m, 2 H), 7.31-7.02 (m, 1 H), 4.87-4.81 (m, 1 H), 4.46 (br t, 1 H), 4.37 (br s, 1 H), 2.60-2.47 (m, 2 H), 2.34 (br d, 1 H), 2.10 (br d, 2 H), 1.94-1.85 (m, 1 H) | m/z 555.2 [M + H]⁺; column: DAICEL CHIRALCEL OD (250 mm* 30 mm, 10 um); mobile phase: [0.1% NH₃H₂O EtOH]; B %: 30%- 30%, min); Rt = 3.590 min 99.5% ee |
| 109 | 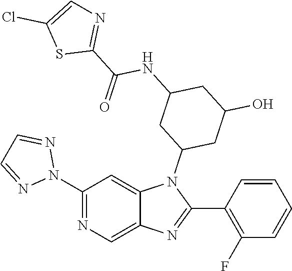 [SMILES: O=C(NC1CC(O)CC(n2c(-c3ccccc3F)nc3cnc(-n4nccn4)cc32)C1)c1ncc(Cl)s1] | (400 MHz, CD₃OD) δ 8.95 (s, 1 H), 8.50 (s, 1 H), 8.08 (s, 2 H), 7.79 (s, 1 H), 7.77-7.69 (m, 2 H), 7.52-7.40 (m, 2 H), 4.40 (br t, 1 H), 4.10-4.00 (m, 1 H), 3.81-3.72 (m, 1 H), 2.48 (q, 1 H), 2.33 (br s, 1 H), 2.26 (q, 3 H), 1.67 (q, 1 H) | m/z 539.1 [M + H]⁺ |
| 110 | 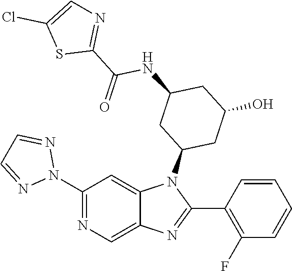 [SMILES: O=C(N[C@H]1C[C@H](O)C[C@@H](n2c(-c3ccccc3F)nc3cnc(-n4nccn4)cc32)C1)c1ncc(Cl)s1] | (400 MHz, CD₃OD) δ 8.94 (s, 1 H), 8.48 (s, 1 H), 8.08 (s, 2 H), 7.80 (s, 1 H), 7.76-7.68 (m, 2 H), 7.50-7.41 (m, 2 H), 4.86-4.82 (m, 1 H), 4.48-4.39 (m, 1 H), 4.36 (br s, 1 H), 2.57-2.46 (m, 2 H), 2.32 (br d, 1 H), 2.10 (br t, 2 H), 1.92-1.83 (m, 1 H) | m/z 539.1 [M + H]⁺; (column: DAICEL CHIRALCEL OJ(250 mm*30 mm, 10 um); mobile phase: [0.1% NH₃H₂O EtOH]; B %: 30%- 30%, min); Rt = 3.558 min 99.5% ee |
| 111 | 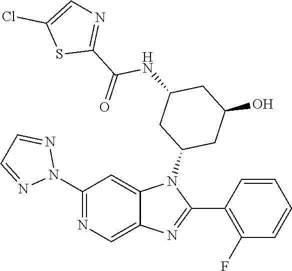 [SMILES: O=C(N[C@@H]1C[C@@H](O)C[C@H](n2c(-c3ccccc3F)nc3cnc(-n4nccn4)cc32)C1)c1ncc(Cl)s1] | (400 MHz, CD₃OD) δ 8.94 (s, 1 H), 8.48 (s, 1 H), 8.08 (s, 2 H), 7.80 (s, 1 H), 7.77-7.69 (m, 2 H), 7.50-7.41 (m, 2 H), 4.86-4.82 (m, 1 H), 4.43 (br t, 1 H), 4.36 (br s, 1 H), 2.59- 2.56 (m, 1 H), 2.56-2.47 (m, 1 H), 2.32 (br d, 1 H), 2.10 (br t, 2 H), 1.92-1.84 (m, 1 H) | m/z 539.2 [M + H]⁺; (column: DAICEL CHIRALCEL OJ(250 mm*30 mm, 10 um); mobile phase: [0.1% NH₃H₂O EtOH]; B %: 30%- 30%,min); Rt = 2.939 min 100% ee |
| 113 | 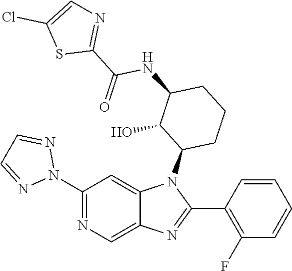 [SMILES: O=C(N[C@H]1CCC[C@@H](n2c(-c3ccccc3F)nc3cnc(-n4nccn4)cc32)[C@@H]1O)c1ncc(Cl)s1] | (400 MHz, CDCl₃) δ 8.42 (br s, 1 H), 7.96 (s, 2 H), 7.91-7.85 (m, 2 H), 7.74 (br s, 1 H), 7.65- 7.58 (m, 1 H), 7.57 (s, 1 H), 7.40 (t, 1 H), 7.31-7.27 (m, 1 H), 5.60 (br s, 1 H), 4.43 (br s, 1 H), 4.24-4.14 (m, 1 H), 3.92 (br d, 1 H), 2.41 (br s, 1 H), 2.28 (br s, 2 H), 1.97 (br s, 1 H), 1.55-1.53 (m, 2 H) | m/z 539.2 [M + H]⁺; (column: DAICEL CHIRALPAK AD (250 mm*30 mm, 10 um); mobile phase: [0.1% NH₃H₂O EtOH]; B %: 40%-40%, min); Rt = 0.631 min 99.9% ee |
| 114 | 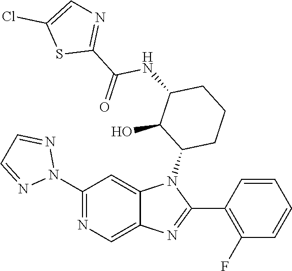 [SMILES: O=C(N[C@@H]1CCC[C@H](n2c(-c3ccccc3F)nc3cnc(-n4nccn4)cc32)[C@H]1O)c1ncc(Cl)s1] | (400 MHz, CDCl₃) δ 8.51 (s, 1 H), 7.95 (s, 2 H), 7.92 (s, 1 H), 7.86 (br t, 1 H), 7.69 (br d, 1 H), 7.64-7.58 (m, 1 H), 7.57 (s, 1 H), 7.37-7.41 (m, 1 H), 7.29-7.25 (m, 1 H), 5.28 (br s, 1 H), 4.43-4.37 (br d, 1 H), 4.21-4.14 (m, 1 H), 3.91 (br s, 1 H), 2.36- 2.28 (m, 3 H), 1.98 (br s, 1 H), 1.59-1.56 (m, 2 H) | m/z 539.2 [M + H]⁺; (column: DAICEL CHIRALPAK AD (250 mm*30 mm, 10 um); mobile phase: [0.1% NH₃H₂O EtOH]; B %: 40%-40%, min); Rt = 0.283 min 98.4% ee |
| 125 | 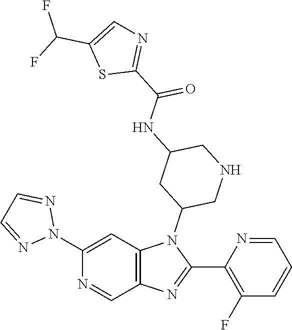 [SMILES: O=C(NC1CNCC(n2c(-c3ncccc3F)nc3cnc(-n4nccn4)cc32)C1)c1ncc(C(F)F)s1] | (400 MHz, CD₃OD) δ 9.01 (s, 1 H), 8.75 (d, 1 H), 8.60 (s, 1 H), 8.17 (s, 1 H), 8.12 (s, 2 H), 7.98 (t, 1 H), 7.80-7.79 (m, 1 H), 7.19 (t, 1 H), 5.01 (br s, 1 H), 4.19 (br s, 1 H), 3.45-3.30 (m, 2 H), 3.24-3.20 (m, 1 H), 2.80-2.69 (m, 2 H), 2.54-2.51 (m, 1 H) | m/z 541.2 [M + H]⁺ |
| | (relative stereochemistry at | | |
| | centers 3 and 5 of piperidine | | |
| | ring is cis) | | |
| 129 | 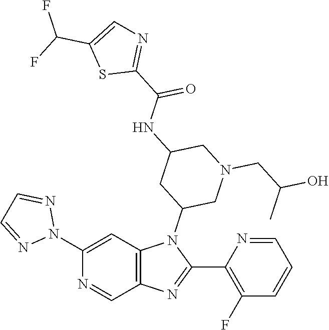 [SMILES: CC(O)CN1CC(NC(=O)c2ncc(C(F)F)s2)CC(n2c(-c3ncccc3F)nc3cnc(-n4nccn4)cc32)C1] | (400 MHz, CD₃OD) δ 9.01 (s, 1 H), 8.72 (t, 1 H), 8.60 (s, 1 H), 8.16 (t, 1 H), 8.11 (s, 2 H), 7.97 (t, 1 H), 7.80-7.76 (m, 1 H), 7.19 (t, 1 H), 5.04 (br s, 1 H), 4.29 (br s, 1 H), 3.96-3.95 (m, 1H), 3.39-3.42 (m, 1 H), 3.24-3.17 (m, 1 H), 3.07-3.00 (m, 1 H), 2.66-2.63 (m, 1 H), 2.53-2.39 (m, 4 H), 1.16 (t, 3 H) | m/z 599.3 [M + H]⁺ |
| | (relative stereochemistry at | | |
| | centers 3 and 5 of piperidine | | |
| | ring is cis) | | |
| 137 | 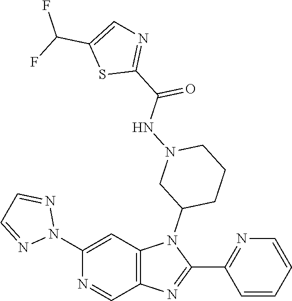 [SMILES: O=C(NN1CCCC(n2c(-c3ccccn3)nc3cnc(-n4nccn4)cc32)C1)c1ncc(C(F)F)s1] | (400 MHz, CDCl₃) δ 9.09 (s, 1 H), 8.80 (d, 1 H), 8.34 (s, 1 H), 8.30-8.26 (m, 2 H), 7.97- 7.94 (m, 3 H), 7.47 (dd, 1 H), 7.06-6.77 (m, 1 H), 6.04-5.94 (m, 1 H), 3.66-3.59 (m, 2 H), 3.39 (br d, 1 H), 2.96-2.91 (m, 1 H), 2.37-2.28 (m, 2 H), 2.06-2.00 (m, 2 H) | m/z 523.2 [M + H]⁺ |
| 142 | 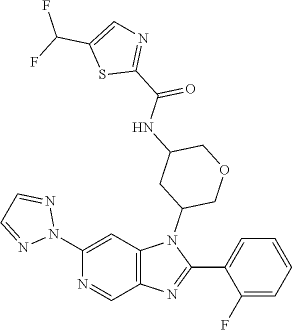 [SMILES: O=C(NC1COCC(n2c(-c3ccccc3F)nc3cnc(-n4nccn4)cc32)C1)c1ncc(C(F)F)s1] | (400 MHz, CD₃OD) δ 8.94 (s, 1 H), 8.58 (s, 1 H), 8.12 (s, 1 H), 8.09 (s, 2 H), 7.77-7.72 (m, 2 H), 7.52-7.44 (m, 2 H), 7.31-7.03 (m, 1 H), 4.51 (br t, 1 H), 4.28-4.21 (m, 1 H), 4.15 (br d, 1 H), 4.10- 4.02 (m, 2 H), 3.56 (t, 1 H), 2.85 (q, 1 H), 2.44 (br d, 1 H) | m/z 541.3 [M + H]⁺ |
| | (relative stereochemistry at | | |
| | centers 3 and 5 of | | |
| | tetrahydropyranyl ring is cis) | | |
| 143 | 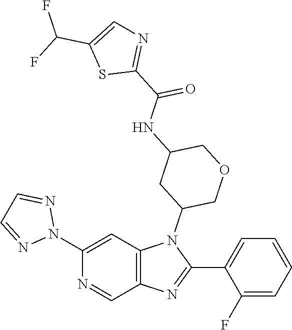 [SMILES: O=C(NC1COCC(n2c(-c3ccccc3F)nc3cnc(-n4nccn4)cc32)C1)c1ncc(C(F)F)s1] | (400 MHz, CD₃OD) δ 8.94 (s, 1 H), 8.57 (s, 1 H), 8.22-8.18 (m, 1 H), 8.09 (s, 2 H), 7.69- 7.61 (m, 1 H), 7.56-7.48 (m, 1 H), 7.40-7.12 (m, 2 H), 6.99 (br s, 1 H), 4.61 (s, 1 H), 4.40- 4.30 (m, 2 H), 4.09 (br d, 1 H), 3.97-4.04 (m, 2 H), 2.71-2.83 (m, 1 H), 2.56 (br d, 1 H) | m/z 541.3 [M + H]⁺ |
| | (relative stereochemistry at | | |
| | centers 3 and 5 of | | |
| | tetrahydropyranyl ring is | | |
| | trans) | | |
| 144 | 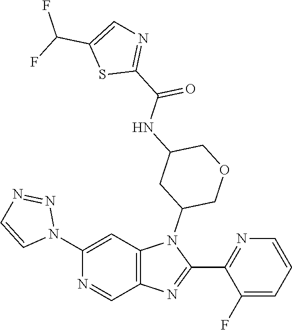 [SMILES: O=C(NC1COCC(n2c(-c3ncccc3F)nc3cnc(-n4ccnn4)cc32)C1)c1ncc(C(F)F)s1] | (400 MHz, CD₃OD) δ 9.01 (s, 1 H), 8.88 (d, 1 H), 8.72 (d, 1 H), 8.68 (s, 1 H), 8.14 (s, 1 H), 7.99-7.92 (m, 2 H), 7.76 (dt, 1 H), 7.32-7.01 (m, 1 H), 5.11-5.03 (m, 1 H), 4.31-4.18 (m, 3 H), 4.06 (br dd, 1 H), 3.58 (t, 1 H), 2.85 (q, 1 H), 2.52 (br d, 1 H) | m/z 542.3 [M + H]⁺ |
| | (relative stereochemistry at | | |
| | centers 3 and 5 of | | |
| | tetrahydropyranyl ring is cis) | | |
| 145 | 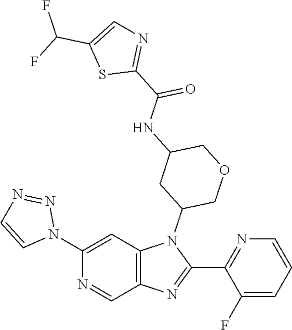 [SMILES: O=C(NC1COCC(n2c(-c3ncccc3F)nc3cnc(-n4ccnn4)cc32)C1)c1ncc(C(F)F)s1] | (400 MHz, CD₃OD) δ 9.00 (s, 1 H), 8.87 (s, 1 H), 8.68 (s, 1 H), 8.36 (d, 1 H), 8.21 (s, 1 H), 7.96 (s, 1 H), 7.86-7.80 (m, 1 H), 7.57 (dt, 1 H), 7.38- 7.10 (m, 1 H), 5.32-5.21 (m, 1 H), 4.40 (br d, 2 H), 4.19 (br dd, 1 H), 4.03 (br d, 2 H), 2.80 (td, 1 H), 2.60 (br d, 1 H) | m/z 542.3 [M + H]⁺ |
| | (relative stereochemistry at | | |
| | centers 3 and 5 of | | |
| | tetrahydropyranyl ring is | | |
| | trans) | | |
| 146 | 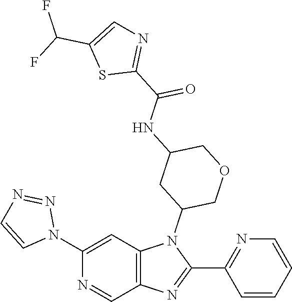 [SMILES: O=C(NC1COCC(n2c(-c3ccccn3)nc3cnc(-n4ccnn4)cc32)C1)c1ncc(C(F)F)s1] | (400 MHz, DMSO-d₆) δ 9.27 (br d, 1 H), 9.08 (s, 1 H), 8.92 (s, 1 H), 8.86 (br d, 1 H), 8.55 (s, 1 H), 8.34 (s, 1 H), 8.28 (d, 1 H), 8.14-8.08 (m, 1 H), 8.04 (s, 1 H), 7.66 (dd, 1 H), 7.61- 7.31 (m, 1 H), 5.88 (br t, 1 H), 4.24-4.14 (m, 2 H), 3.99-4.06 (m, 1 H), 3.92 (br dd, 1 H), 3.56 (br t, 1 H), 3.01-2.91 (m, 1 H), 2.41-2.33 (m, 1 H) | m/z 524.3 [M + H]⁺ |
| | (relative stereochemistry at | | |
| | centers 3 and 5 of | | |
| | tetrahydropyranyl ring is cis) | | |
| 147 | 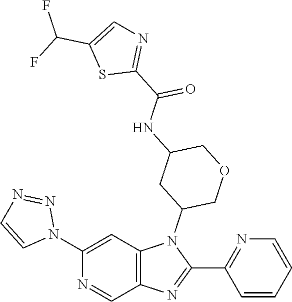 [SMILES: O=C(NC1COCC(n2c(-c3ccccn3)nc3cnc(-n4ccnn4)cc32)C1)c1ncc(C(F)F)s1] | (400 MHz, DMSO-d₆) δ 9.07 (s, 1 H), 8.90 (s, 1 H), 8.75 (s, 1 H), 8.67 (br d, 1 H), 8.54 (d, 1 H), 8.43 (s, 1 H), 8.27 (d, 1 H), 8.09-8.04 (m, 1 H), 8.03 (s, 1 H), 7.65-7.48 (m, 2 H), 6.07 (br s, 1 H), 4.37 (t, 1 H), 4.27 (br s, 1 H), 4.13 (br d, 1 H), 4.03 (br d, 1 H), 3.95- 3.90 (m, 1 H), 3.93 (br d, 1 H), 2.81-2.74 (m, 1 H), 2.67 (s, 1 H) | m/z 524.4 [M + H]⁺ |
| | (relative stereochemistry at | | |
| | centers 3 and 5 of | | |
| | tetrahydropyranyl ring is | | |
| | trans) | | |
| 193 | 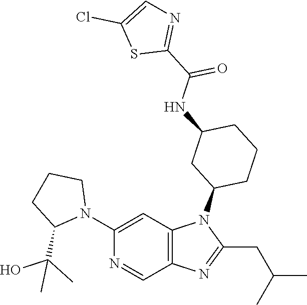 [SMILES: CC(C)Cc1nc2cnc(N3CCC[C@H]3C(C)(C)O)cc2n1[C@@H]1CCC[C@H](NC(=O)c2ncc(Cl)s2)C1] | (400 MHz, DMSO-d₆) δ 8.91 (d, 1H), 8.32 (s, 1H), 8.08 (s, 1H), 6.61 (s, 1H), 4.55-4.42 (m, 1H), 4.21-4.10 (m, 2H), 3.65-3.56 (m, 1H), 2.80-2.72 (m, 2H), 2.19-1.80 (m, 11H), 1.78-1.46 (m, 4H), 1.16 (s, 3H), 1.02 (s, 3H), 0.99-0.95 (m, 6H). | m/z 545.3 [M + H]⁺ |
| 194 | 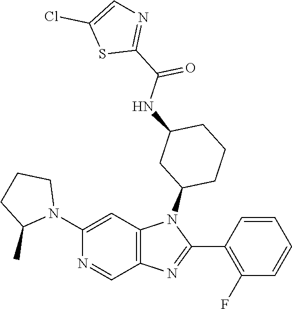 [SMILES: C[C@H]1CCCN1c1cc2c(cn1)nc(-c1ccccc1F)n2[C@@H]1CCC[C@H](NC(=O)c2ncc(Cl)s2)C1] | (400 MHz, DMSO-d₆) δ 8.98 (d, 1H), 8.54 (s, 1H), 8.07 (s, 1H), 7.70-7.60 (m, 2H), 7.50- 7.38 (m, 2H), 6.44 (s, 1H), 4.35-4.24 (m, 1H), 4.07-3.96 (m, 1H), 3.92-3.80 (m, 1H), 3.56-3.51 (m, 1H), 3.37-3.36 (m, 1H), 2.48-2.42 (m, 1H), 2.15-1.90 (m, 5H), 1.86-1.70 (m, 4H), 1.55-1.46 (m, 1H), 1.42-1.32 (m, 1H), 1.23 (d, 3H). | m/z 539.3 [M + H]⁺ |
| 195 | 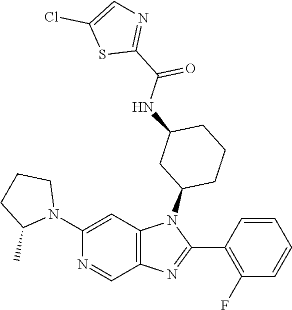 [SMILES: C[C@@H]1CCCN1c1cc2c(cn1)nc(-c1ccccc1F)n2[C@@H]1CCC[C@H](NC(=O)c2ncc(Cl)s2)C1] | (400 MHz, DMSO-d₆) δ 8.90 (d, 1H), 8.55 (s, 1H), 8.07 (s, 1H), 7.70-7.60 (m, 2H), 7.49- 7.38 (m, 2H), 6.45 (s, 1H), 4.34-4.24 (m, 1H), 4.09-3.98 (m, 1H), 3.96-3.84 (m, 1H), 3.64-3.55 (m, 1H), 3.32-3.29 (m, 1H), 2.61-2.53 (m, 1H), 2.16-1.95 (m, 4H), 1.95-1.71 (m, 5H), 1.57-1.37 (m, 2H), 1.22 (d, 3H). | m/z 539.3 [M + H]⁺ |
| 221 | 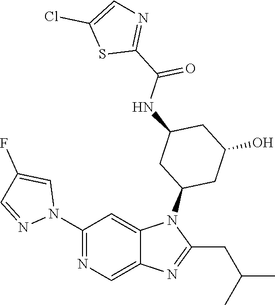 [SMILES: CC(C)Cc1nc2cnc(-n3cc(F)cn3)cc2n1[C@@H]1C[C@@H](O)C[C@H](NC(=O)c2ncc(Cl)s2)C1] | (400 MHz, CD₃OD) δ 8.67 (s, 1 H), 8.56-8.55 (m, 1 H), 8.14 (s, 1 H), 7.80 (s, 1 H), 7.72- 7.71 (m, 1 H), 5.04-4.97 (m, 1 H), 4.64-4.59 (m, 1 H), 4.43 (br s, 1 H), 2.97-2.90 (m, 2 H), 2.52-2.49 (m, 2 H), 2.27-2.12 (m, 3 H), 1.94-1.86 (m, 2 H), 1.08-1.05 (m, 6 H). | m/z 518.2 [M + H]⁺ (column: DAICEL CHIRALPAK IC(250 mm*30 mm, 10 um); mobile phase: [0.1% NH₃H₂O MeOH]; B %: 40%-40%, min) Rt = 2.084 min 100% ee |
| 222 | 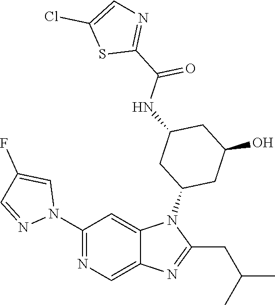 [SMILES: CC(C)Cc1nc2cnc(-n3cc(F)cn3)cc2n1[C@H]1C[C@H](O)C[C@@H](NC(=O)c2ncc(Cl)s2)C1] | (400 MHz, CD₃OD) δ 8.67 (s, 1 H), 8.56-8.55 (m, 1 H), 8.14 (s, 1 H), 7.80 (s, 1 H), 7.72- 7.71 (m, 1 H), 5.04-4.97 (m, 1 H), 4.64-4.59 (m, 1 H), 4.43 (br s, 1 H), 2.97-2.90 (m, 2 H), 2.52-2.49 (m, 2 H), 2.27-2.12 (m, 3 H), 1.94-1.86 (m, 2 H), 1.08-1.05 (m, 6 H). | m/z 518.2 [M + H]⁺ (column: DAICEL CHIRALPAK IC(250 mm*30 mm, 10 um); mobile phase: [0.1% NH₃H₂O MeOH]; B %: 40%-40%, min) Rt = 2.819 min 99.6% ee |
| 227 | 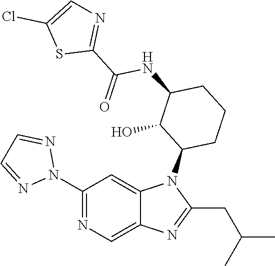 [SMILES: CC(C)Cc1nc2cnc(-n3nccn3)cc2n1[C@@H]1CCC[C@H](NC(=O)c2ncc(Cl)s2)[C@H]1O] | (400 MHz, CD₃OD) δ 8.78 (s, 1 H), 8.37 (s, 1 H), 8.05 (s, 1 H), 7.79 (s, 1 H), 4.44-4.42 (m, 2 H), 4.12-4.10 (m, 1 H), 3.04-2.88 (m, 2 H), 2.42-2.33 (m, 2 H), 2.09-2.03 (m, 2 H), 1.81-1.75 (m, 2 H), 1.10-1.05 (m, 6 H). | m/z 501.2 [M + H]⁺ (column: DAICEL CHIRALPAK AD(250 mm*30 mm, 10 um); mobile phase: [0.1% NH₃H₂O EtOH]; B %: 45%- 45%, min) Rt = 3.224 min 99.0% ee |
| 228 | 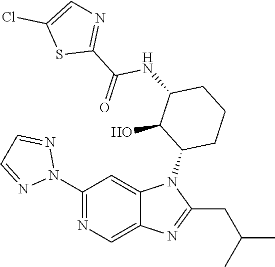 [SMILES: CC(C)Cc1nc2cnc(-n3nccn3)cc2n1[C@H]1CCC[C@@H](NC(=O)c2ncc(Cl)s2)[C@@H]1O] | (400 MHz, CD₃OD) δ 8.77 (s, 1 H), 8.37 (s, 1 H), 8.05 (s, 1 H), 7.79 (s, 1 H), 4.44-4.39 (m, 2 H), 4.12-4.10 (m, 1 H), 3.03-2.87 (m, 2 H), 2.42-2.32 (m, 2 H), 2.09-1.99 (m, 2 H), 1.81-1.75 (m, 2 H), 1.10-1.05 (m, 6 H). | m/z 501.2 [M + H]⁺ (column: DAICEL CHIRALPAK AD(250 mm*30 mm, 10 um); mobile phase: [0.1% NH₃H₂O EtOH]; B %: 45%- 45%, min) Rt = 1.083 min 100% ee |
| 231 | 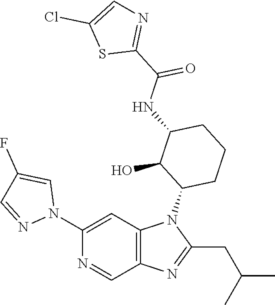 [SMILES: CC(C)Cc1nc2cnc(-n3cc(F)cn3)cc2n1[C@H]1CCC[C@@H](NC(=O)c2ncc(Cl)s2)[C@@H]1O] | (400 MHz, CDCl₃) δ 8.71 (s, 1 H), 8.43-8.42 (m, 1 H), 7.85 (s, 1 H), 7.59 (s, 1 H), 7.50-7.48 (m, 1 H), 7.31-7.29 (m, 1 H), 4.32-4.19 (m, 4 H), 2.83-2.81 (m, 2 H), 2.37-2.20 (m, 3 H), 2.04-1.98 (m, 2 H), 1.68-1.66 (m, 2 H), 1.04-1.02 (m, 6 H). | m/z 518.1 [M + H]⁺ (column: Phenomenex- Cellulose-2 (250 mm*30 mm, 10 um); mobile phase: [0.1% NH₃H₂O MeOH]; B %: 35%-35%, min) Rt = 2.238 min 98.4% ee |
| 232 | 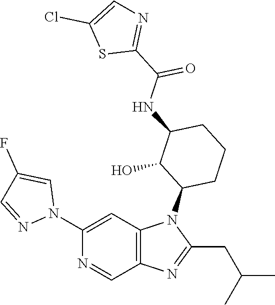 [SMILES: CC(C)Cc1nc2cnc(-n3cc(F)cn3)cc2n1[C@@H]1CCC[C@H](NC(=O)c2ncc(Cl)s2)[C@H]1O] | (400 MHz, CDCl₃) δ 8.71 (s, 1 H), 8.43-8.42 (m, 1 H), 7.86 (s, 1 H), 7.59 (s, 1 H), 7.49-7.48 m, 1 H), 7.33-7.31 (m, 1 H), 4.31-4.24 (m, 3 H), 4.10 (br s, 1 H), 2.83-2.81 (m, 2 H), 2.38- 2.20 (m, 3 H), 2.05-2.00 (m, 2 H), 1.70-1.65 (m, 1 H), 1.03- 1.01 (m, 6 H). | m/z 518.1 [M + H]⁺ (column: Phenomenex- Cellulose-2 (250 mm*30 mm, 10 um); mobile phase: [0.1% NH₃H₂O MeOH]; B %: 35%-35%, min) Rt = 1.537 min 99.8% ee |
| 243 | 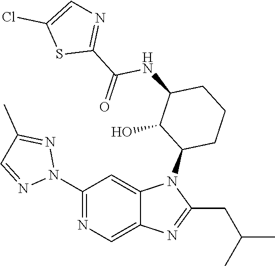 [SMILES: Cc1cnn(-c2cc3c(cn2)nc(CC(C)C)n3[C@@H]2CCC[C@H](NC(=O)c3ncc(Cl)s3)[C@H]2O)n1] | (400 MHz, CDCl₃) δ 8.34 (s, 1 H), 7.72-7.66 (m, 3 H), 7.58 (s, 1 H), 5.41 (br s, 1 H), 4.46- 4.41 (m, 1 H), 4.33-4.28 (m, 1 H), 4.16-4.13 (m, 1 H), 2.90- 2.87 (m, 2 H), 2.43 (s, 3 H), 2.35-2.31 (m, 2 H), 2.25-2.00 (m, 2 H), 1.73-1.70 (m, 1 H), 1.11-1.07 (m, 6 H). | m/z 515.2 [M + H]⁺ (column: DAICEL CHIRALPAK AD(250 mm*30 mm, 10 um); mobile phase: [0.1% NH₃H₂O ETOH]; B %: 40%-40%,min) Rt = 2.764 min 99.9% ee |
| 244 | 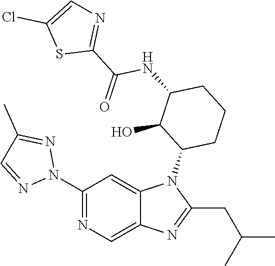 [SMILES: Cc1cnn(-c2cc3c(cn2)nc(CC(C)C)n3[C@H]2CCC[C@@H](NC(=O)c3ncc(Cl)s3)[C@@H]2O)n1] | (400 MHz, CDCl₃) δ 8.37 (br s, 1 H), 7.71 (br s, 1 H), 7.68- 7.65 (br s, 2 H), 7.58 (s, 1 H), 5.33 (br s, 1 H), 4.45-4.40 (m, 1 H), 4.33-4.29 (m, 1 H), 4.15- 4.13 (m, 1 H), 2.90-2.88 (m, 2 H), 2.44 (s, 3 H), 2.41 (s, 1 H), 2.35-2.31 (m, 2 H), 2.01 (br s, 2 H), 1.61 (br s, 1 H), 1.11-1.07 (m, 6 H). | m/z 515.2 [M + H]⁺ (column: DAICEL CHIRALPAK AD(250 mm*30 mm, 10 um); mobile phase: [0.1% NH₃H₂O ETOH]; B %: 40%-40%,min) Rt = 1.056 min 100% ee |
| 246 | 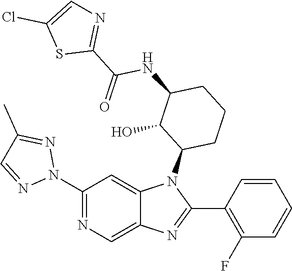 [SMILES: Cc1cnn(-c2cc3c(cn2)nc(-c2ccccc2F)n3[C@@H]2CCC[C@H](NC(=O)c3ncc(Cl)s3)[C@H]2O)n1] | (400 MHz, CDCl₃) δ 8.72 (br s, 1 H), 7.97 (br s, 1 H), 7.83- 7.81 (m, 1 H), 7.69 (s, 1 H), 7.60-7.57 (m, 2 H), 7.48-7.46 (m, 1 H), 7.38-7.37 (m, 1 H), 7.28-7.23 (m, 1 H), 4.40-4.35 (m, 1 H), 4.19-4.14 (m, 1 H), 3.93-3.89 (m, 1 H), 2.47 (s, 3 H), 2.37-2.28 (m, 3 H), 2.04- 2.01 (m, 1 H), 1.63-1.60 (m, 2 H). | m/z 553.2 [M + H]⁺ (column: DAICEL CHIRALPAK AD(250 mm*30 mm, 10 um); mobile phase: [0.1% NH₃H₂O EtOH]; B %: 40%- 40%, min) Rt = 2.536 min 100% ee |
| 247 | 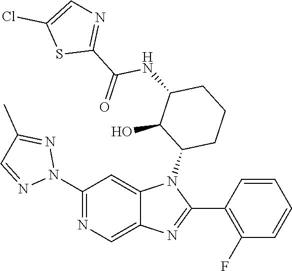 [SMILES: Cc1cnn(-c2cc3c(cn2)nc(-c2ccccc2F)n3[C@H]2CCC[C@@H](NC(=O)c3ncc(Cl)s3)[C@@H]2O)n1] | (400 MHz, CDCl₃) δ 8.60 (br s, 1 H), 7.90 (br s, 1 H), 7.83- 7.82 (m, 1 H), 7.69 (s, 1 H), 7.62-7.57 (m, 3 H), 7.38-7.36 m, 1 H), 7.27-7.23 (m, 1 H), 4.44-4.39 (m, 1 H), 4.20-4.14 (m, 1 H), 3.92 (br s, 1 H), 2.47 (s, 3 H), 2.35-2.30 (m, 3 H), 2.01 (br s, 1 H), 1.64-1.62 (m, 2 H). | m/z 553.2 [M + H]⁺ (column: DAICEL CHIRALPAK AD(250 mm*30 mm, 10 um); mobile phase: [0.1% NH₃H₂O EtOH]; B %: 40%- 40%, min) Rt = 1.272 min 100% ee |
| 304 | 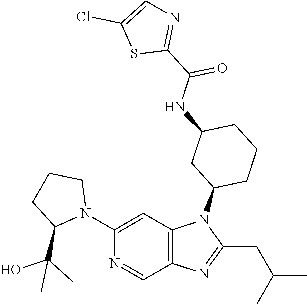 [SMILES: CC(C)Cc1nc2cnc(N3CCC[C@@H]3C(C)(C)O)cc2n1[C@@H]1CCC[C@H](NC(=O)c2ncc(Cl)s2)C1] | (400 MHz, DMSO-d₆) δ 8.96 (d, 1H), 8.36 (s, 1H), 8.08 (s, 1H), 6.63 (s, 1H), 4.55-4.43 (m, 1H), 4.20-4.10 (m, 2H), 3.64-3.54 (m, 1H), 3.48-3.40 (m, 1H), 2.76 (d, 2H), 2.20- 1.81 (m, 10H), 1.78-1.70 (m, 1H), 1.66-1.51 (m, 2H), 1.17 (s, 3H), 1.03 (s, 3H), 1.00-0.94 (m, 6H). | m/z 545.2 [M + H]⁺ |
| 307 | 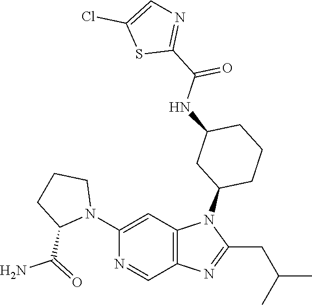 [SMILES: CC(C)Cc1nc2cnc(N3CCC[C@H]3C(N)=O)cc2n1[C@@H]1CCC[C@H](NC(=O)c2ncc(Cl)s2)C1] | (400 MHz, DMSO-d₆) δ 9.02- 8.83 (m, 1H), 8.39-8.30 (m, 1H), 8.08 (s, 1H), 7.31-7.23 (m, 1H), 6.96-6.81 (m, 1H), 6.49-6.44 (m, 1H), 4.51-4.26 (m, 2H), 4.23-4.06 (m, 1H), 3.73-3.59 (m, 1H), 3.45-3.35 (m, 1H), 2.73 (d, 2H), 2.41- 2.23 (m, 1H), 2.20-1.93 (m, 6H), 1.93-1.79 (m, 3H), 1.76- 1.49 (m, 3H), 1.00-0.92 (m, 6H). | m/z 530.3 [M + H]⁺ |
| 308 | 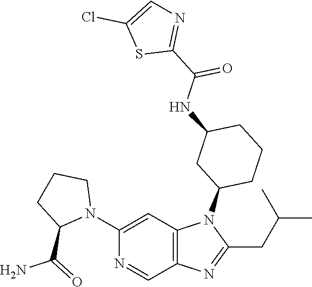 [SMILES: CC(C)Cc1nc2cnc(N3CCC[C@@H]3C(N)=O)cc2n1[C@@H]1CCC[C@H](NC(=O)c2ncc(Cl)s2)C1] | (400 MHz, DMSO-d₆) δ 8.98- 8.83 (m, 1H), 8.37-8.32 (m, 1H), 8.12-8.05 (m, 1H), 7.30- 7.24 (m, 1H), 6.94-6.82 (m, 1H), 6.51-6.43 (m, 1H), 4.51- 4.26 (m, 2H), 4.21-4.08 (m, 1H), 3.73-3.58 (m, 1H), 3.45- 3.37 (m, 1H), 2.74 (d, 2H), 2.14-1.68 (m, 12H), 1.62-1.55 (m, 1H), 0.97 (d, 6H). | m/z 530.2 [M + H]⁺ |
| 309 | 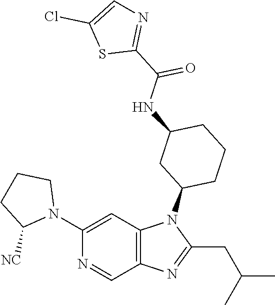 [SMILES: CC(C)Cc1nc2cnc(N3CCC[C@H]3C#N)cc2n1[C@@H]1CCC[C@H](NC(=O)c2ncc(Cl)s2)C1] | (400 MHz, DMSO-d₆) δ 8.94- 8.80 (m, 1H), 8.48 (s, 1H), 8.08 (d, 1H), 6.61 (s, 1H), 5.05-4.98 (m, 1H), 4.58-4.45 (m, 1H), 4.21-4.09 (m, 1H), 3.62-3.39 (m, 2H), 2.77 (d, 2H), 2.35-2.27 (m, 2H), 2.23- 2.04 (m, 5H), 1.93-1.85 (m, 3H), 1.78-1.70 (m, 1H), 1.65- 1.53 (m, 2H), 0.99-0.94 (m, 6H). | m/z 512.1 [M + H]⁺ |
| 310 | 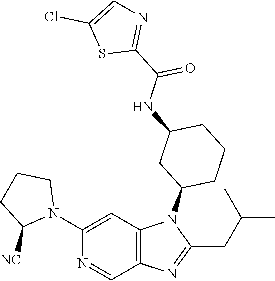 [SMILES: CC(C)Cc1nc2cnc(N3CCC[C@@H]3C#N)cc2n1[C@@H]1CCC[C@H](NC(=O)c2ncc(Cl)s2)C1] | (400 MHz, DMSO-d₆) δ 8.93- 8.79 (m, 1H), 8.48 (s, 1H), 8.10-8.05 (m, 1H), 6.61 (s, 1H), 5.06-4.97 (m, 1H), 4.55- 4.43 (m, 1H), 4.21-4.10 (m, 1H), 3.60-3.39 (m, 2H), 2.77 (d, 2H), 2.34-2.27 (m, 2H), 2.23-2.02 (m, 5H), 1.92-1.84 (m, 3H), 1.78-1.70 (m, 1H), 1.64-1.50 (m, 2H), 1.01-0.96 (m, 6H). | m/z 512.3 [M + H]⁺ |
| 15 | 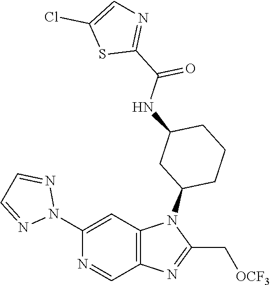 [SMILES: O=C(N[C@H]1CCC[C@@H](n2c(COC(F)(F)F)nc3cnc(-n4nccn4)cc32)C1)c1ncc(Cl)s1] | (400 MHz, DMSO-d₆) δ 9.04 (d, 1H), 9.00 (s, 1H), 8.27 (s, 1H), 8.20 (s, 2H), 8.06 (s, 1H), 5.79-5.61 (m, 2H), 4.75-4.62 (m, 1H), 4.20-4.10 (m, 1H), 2.66-2.55 (m, 1H), 2.14-1.85 (m, 5H), 1.68-1.50 (m, 2H). | m/z 527.1 [M + H]⁺ |
| 334 | 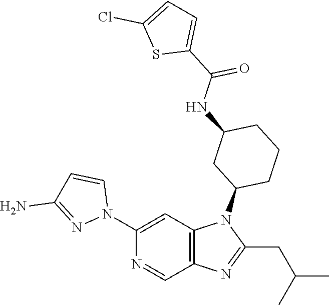 [SMILES: CC(C)Cc1nc2cnc(-n3ccc(N)n3)cc2n1[C@@H]1CCC[C@H](NC(=O)c2ccc(Cl)s2)C1] | (400 MHz, DMSO-d₆) δ 9.18 (d, 1H), 8.61 (s, 1H), 8.28 (d, 1H), 8.05 (s, 1H), 7.77 (s, 1H), 5.77 (d, 1H), 5.17 (s, 2H), 4.63-4.51 (m, 1H), 4.24-4.10 (m, 1H), 2.84 (d, 2H), 2.25- 1.82 (m, 7H), 1.72-1.53 (m, 2H), 1.01 (d, 6H). | m/z 499.2 [M + H]⁺ |
| 346 | 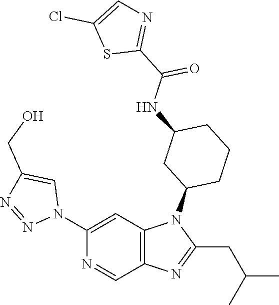 [SMILES: CC(C)Cc1nc2cnc(-n3cc(CO)nn3)cc2n1[C@@H]1CCC[C@H](NC(=O)c2ncc(Cl)s2)C1] | (400 MHz, CDC1₃) δ 8.85 (s, 1 H), 8.62 (s, 1 H), 8.27 (s, 1 H), 7.65 (s, 1 H), 7.24-7.22 (m, 1 H), 4.93 (s, 2 H), 4.41- 4.39 (m, 1 H), 4.22-4.20 (m, 1 H), 2.86-2.84 (m, 2 H), 2.41- 2.14 (m, 6 H), 2.02-1.99 (m, 1 H), 1.62-1.57 (m, 2 H), 1.11- 1.09 (m, 6 H). | m/z 515.2 [M + H]⁺ |
| 350 | 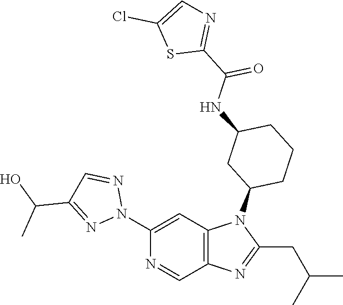 [SMILES: CC(C)Cc1nc2cnc(-n3ncc(C(C)O)n3)cc2n1[C@@H]1CCC[C@H](NC(=O)c2ncc(Cl)s2)C1] | (400 MHz, CDCl₃) δ 8.95 (s, 1 H), 8.09 (s, 1 H), 7.87 (s, 1 H), 7.64 (s, 1 H), 7.22-7.20 (m, 1 H), 5.28-5.23(m, 1 H), 4.41-4.36 (m, 1 H), 4.23-4.19 (m, 1 H), 2.85-2.82 (m, 2 H), 2.41-2.03 (m, 8 H), 1.70-1.68 (m, 3 H), 1.57-1.56 (m, 1 H), 1.11-1.09 (m, 6 H). | m/z 529.1 [M + H]⁺ |
| 357 | 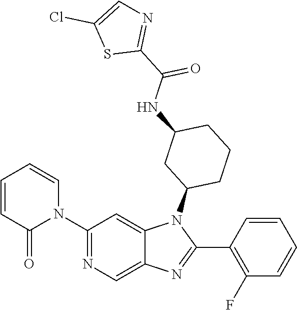 [SMILES: O=C(N[C@H]1CCC[C@@H](n2c(-c3ccccc3F)nc3cnc(-n4ccccc4=O)cc32)C1)c1ncc(Cl)s1] | (400 MHz, DMSO-d₆) δ 9.01 (s, 1H), 8.85 (d, 1H), 8.12 (s, 1H), 8.04 (s, 1H), 7.92 (dd, 1H), 7.79-7.70 (m, 2H), 7.62- 7.44 (m, 3H), 6.55 (d, 1H), 6.40 (t, 1H), 4.29-4.15 (m, 1H), 3.99-3.87 (m, 1H), 2.12- 1.71 (m, 6H), 1.60-1.33 (m, 2H). | m/z 549.1 [M + H]⁺ |
| 359 | 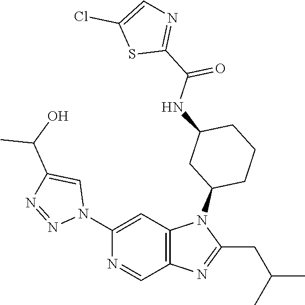 [SMILES: CC(C)Cc1nc2cnc(-n3cc(C(C)O)nn3)cc2n1[C@@H]1CCC[C@H](NC(=O)c2ncc(Cl)s2)C1] | (400 MHz, CDC1₃) δ 8.84 (s, 1 H), 8.56 (s, 1 H), 8.26 (s, 1 H), 7.65 (s, 1 H), 7.25-7.23 (m, 1 H), 5.23-5.18 (m, 1 H), 4.41-4.37 (m, 1 H), 4.23-4.20 (m, 1 H), 2.85-2.83 (m, 2 H), 2.41-2.01 (m, 8 H), 1.69-1.67 (m, 3 H), 1.59-1.58 (m, 1 H), 1.11-1.09 (m, 6 H). | m/z 529.2 [M + H]⁺ |
| 363 | 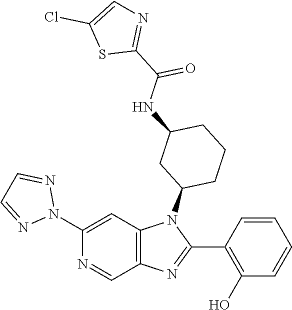 [SMILES: O=C(N[C@H]1CCC[C@@H](n2c(-c3ccccc3O)nc3cnc(-n4nccn4)cc32)C1)c1ncc(Cl)s1] | (400 MHz, DMSO-d₆) δ 10.35 (s, 1H), 9.05 (d, 1H), 8.94 (s, 1H), 8.26 (s, 1H), 8.19 (s, 2H), 8.05 (s, 1H), 7.49-7.42 (m, 2H), 7.08 (d, 1H), 7.01 (t, 1H), 4.24-4.12 (m, 1H), 3.95-3.81 (m, 1H), 2.65-2.53 (m, 1H), 2.15-2.01 (m, 2H), 1.99-1.77 (m, 3H), 1.63-1.52 (m, 1H), 1.40-1.28 (m, 1H). | m/z 521.3 [M + H]⁺ |
| 373 | 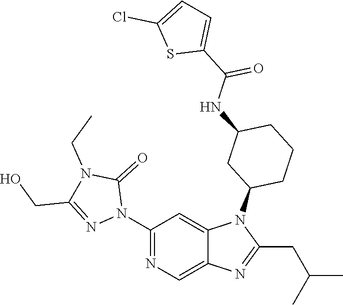 [SMILES: CCn1c(CO)nn(-c2cc3c(cn2)nc(CC(C)C)n3[C@@H]2CCC[C@H](NC(=O)c3ccc(Cl)s3)C2)c1=O] | (400 MHz, CDC1₃) δ 8.82 (s, 1 H), 8.32 (s, 1 H), 7.36-7 35 (m, 1 H), 6.85-6.84 (m, 1 H), 6.67 (br s, 1 H), 4.80-4.76 (m, 2 H), 4.33-4.23 (m, 2 H), 3.99-3.96 (m, 2 H), 2.80-2.78 (m, 2 H), 2.33-2.20 (m, 7 H), 2.10-1.98 (m, 2 H), 1.42-1.39 (m, 3 H), 1.07-1.05 (m, 6 H). | 558.2 [M + H]⁺ |
| 387 | 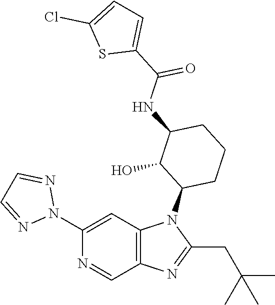 [SMILES: CC(C)(C)Cc1nc2cnc(-n3nccn3)cc2n1[C@@H]1CCC[C@H](NC(=O)c2ccc(Cl)s2)[C@H]1O] | (400 MHz, CD₃OD) δ 8.80 (s, 1H), 8.36 (s, 1H), 8.04 (s, 2H), 7.50 (d, 1H), 6.97 (d, 1H), 4.59-4.46 (m, 1H), 4.26 (t, 1H), 4.15-4.04 (m, 1H), 3.24- 3.17 (m, 1H), 2.90-2.83 (m, 1H), 2.60-2.47 (m, 1H), 2.19- 2.08 (m, 2H), 2.05-1.97 (m, 1H), 1.79-1.66 (m, 2H), 1.14 (s, 9H). | m/z 514.1 [M + H]⁺ (Column: ChiralPak IH (100*4.6 mm, 5 μm); mobile phase: [0.05% DEA MeOH]; B % 0.0 min-1.0 min @ 10% B, 1.0 min-4.5 min gradient (10-40% B), 4.5 min-7.0 min @, 40% B, 7.0 min-8.0 min @ 10% B; Rt = 2.771 min >99% ee |
| 388 | 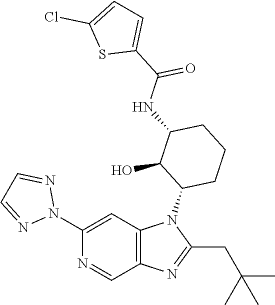 [SMILES: CC(C)(C)Cc1nc2cnc(-n3nccn3)cc2n1[C@H]1CCC[C@@H](NC(=O)c2ccc(Cl)s2)[C@@H]1O] | (400 MHz, CD₃OD) δ 8.80 (s, 1H), 8.37 (s, 1H), 8.04 (s, 2H), 7.51 (d, 1H), 6.98 (d, 1H), 4.57-4.46 (m, 1H), 4.26 (t, 1H), 4.15-4.06 (m, 1H), 3.20 (d, 1H), 2.87 (d, 1H), 2.60-2.48 (m, 1H), 2.20-1.96 (m, 3H), 1.81-1.66 (m, 2H), 1.15 (s, 9H) | m/z 514.1 [M + H]⁺ (Column: ChiralPak IH (100*4.6 mm, 5 μm); mobile phase: [0.05% DEA MeOH]; B % 0.0 min-1.0 min @ 10% B, 1.0 min-4.5 min gradient (10-40% B), 4.5 min-7.0 min @ 40% B, 7.0 min-8.0 min @ 10% B; Rt = 4.432 min >99% ee |
| 393 | 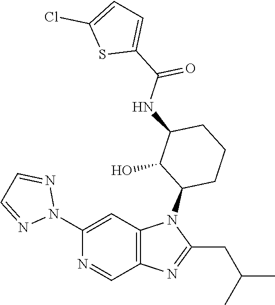 [SMILES: CC(C)Cc1nc2cnc(-n3nccn3)cc2n1[C@@H]1CCC[C@H](NC(=O)c2ccc(Cl)s2)[C@H]1O] | (400 MHz, CDCl₃) δ 8.13 (s, 1 H), 7.83 (s, 2 H), 7.63 (s, 1 H), 7.31-7.30 (m, 1 H), 7.25- 7.23 (m, 1 H), 6.79-6.78 (m, 1 H), 4.41-4.37 (m, 1 H), 4.30- 4.24 (m, 1 H), 4.04-3.97 (m, 1 H), 2.92-2.85 (m, 2 H), 2.61- 2.59 (m, 1 H), 2.38-2.31 (m, 1 H), 2.27-2.17 (m, 1 H), 2.05-1.97 (m, 2 H), 1.51-1.47 (m, 2 H), 1.13-1.08 (m, 6 H). | m/z 500.1 [M + H]⁺; (column: DAICEL CHIRALPAK AD (250 mm*30 mm, 10 um); mobile phase: [0.1% NH₃H₂O EtOH]; B %: 45%-45%, min); Rt = 2.116 min 100% ee |
| 394 | 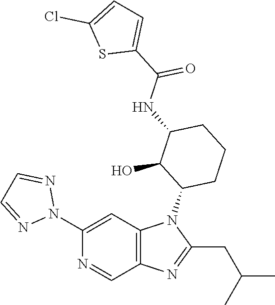 [SMILES: CC(C)Cc1nc2cnc(-n3nccn3)cc2n1[C@H]1CCC[C@@H](NC(=O)c2ccc(Cl)s2)[C@@H]1O] | (400 MHz, CDCl₃) δ 8.16 (br s, 1 H), 7.83 (s, 2 H), 7.65 (br s, 1 H), 7.31-7.30 (dm, 1 H), 7.20-7.19 (m, 1 H), 6.79-6.78 (m, 1 H), 4.41-4.36 (m, 1 H), 4.30-4.24 (m, 1 H), 4.04-3.97 (m, 1 H), 2.96-2.85 (m, 2 H), 2.62-2.59 (m, 1 H), 2.37-2.31 (m, 1 H), 2.28-2.19 (m, 1 H), 2.05-1.97 (m, 2 H), 1.55-1.48 (m, 2 H), 1.13-1.08 (m, 6 H). | m/z 500.2 [M + H]⁺; (column: DAICEL CHIRALPAK AD (250 mm*30 mm, 10 um); mobile phase: [0.1% NH₃H₂O EtOH]; B %: 45%-45%, min). Rt = 1.228 min 100% ee |
| 395 | 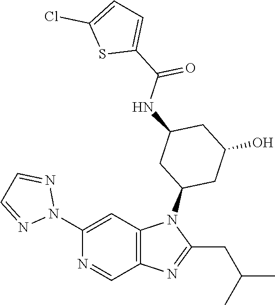 [SMILES: CC(C)Cc1nc2cnc(-n3nccn3)cc2n1[C@@H]1C[C@@H](O)C[C@H](NC(=O)c2ccc(Cl)s2)C1] | (400 MHz, CD₃OD) δ 8.78 (s, 1 H), 8.32 (s, 1 H), 8.04 (s, 2 H), 7.54-7.53 (m, 1 H), 7.00-6.99 (m, 1 H), 5.08-5.02 (m, 1 H), 4.62-4.60 (m, 1 H), 4.40 (br s, 1 H), 3.03-2.92 (m, 2 H), 2.54-2.40 (m, 2 H), 2.32-2.25 (m, 2 H), 2.21-2.17 (m, 1 H), 2.08-2.05 (m, 1 H), 1.84-1.81 (m, 1 H), 1.10-1.07 (m, 6 H). | m/z 500.2 [M + H]⁺; (column: DAICEL CHIRALCEL OD-H (250 mm*30 mm, 5 um); mobile phase: [0.1% NH₃H₂O EtOH]; B %: 35%-35%, min). Rt = 3.862 min 100% ee |
| 396 | 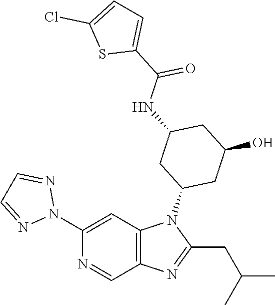 [SMILES: CC(C)Cc1nc2cnc(-n3nccn3)cc2n1[C@H]1C[C@H](O)C[C@@H](NC(=O)c2ccc(Cl)s2)C1] | (400 MHz, CD₃OD) δ 8.78 (s, 1 H), 8.32 (s, 1 H), 8.04 (s, 2 H), 7.54-7.53 (m, 1 H), 7.00- 6.99 (m, 1 H), 5.08-5.02 (m, 1 H), 4.62-4.60 (m, 1 H), 4.44 (br s, 1 H), 3.01-2.95 (m, 2 H), 2.54-2.40 (m, 2 H), 2.32-2.27 (m, 2 H), 2.21-2.17 (m, 1 H), 2.08-2.05 (m, 1 H), 1.87-1.84 (m, 1 H), 1.10-1.07 (m, 6 H). | m/z 500.2 [M + H]⁺; (column: DAICEL CHIRALCEL OD-H (250 mm*30 mm, 5 um); mobile phase: [0.1% NH₃H₂O EtOH]; B %: 35%-35%, min). Rt = 4.252 min 87.7% ee |
| 415 | 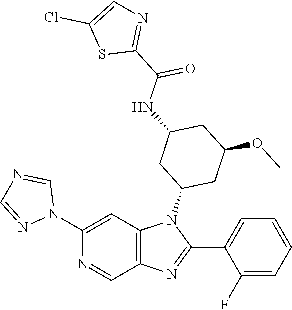 [SMILES: CO[C@@H]1C[C@@H](NC(=O)c2ncc(Cl)s2)C[C@@H](n2c(-c3ccccc3F)nc3cnc(-n4cncn4)cc32)C1] | (400 MHz, CDCl₃) δ 9.29 (s, 1 H), 8.98 (s, 1 H), 8.18 (s, 1 H), 8.09 (s, 1 H), 7.69-7.64 (m, 3 H), 7.44-7.34 (m, 2 H), 7.10- 7.08 (m, 1 H), 4.70 (br s, 1 H), 4.40-4.36 (m, 1 H), 3.88 (s, 1 H), 3.31 (s, 3 H), 2.44-2.28 (m, 5 H), 1.62-1.59 (m, 1 H). | m/z 553.2 [M + H]⁺; (column: REGIS(S,S)WHELK- O1 (250 mm*25 mm, 10 um); mobile phase: [0.1% NH₃H₂O EtOH]; B %: 50%-50%, min). Rt = 3.149 min 99.6% ee |
| 416 | 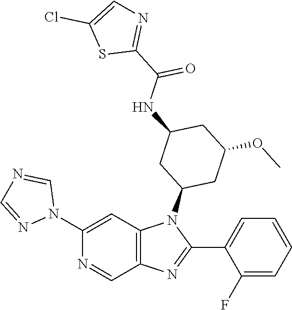 [SMILES: CO[C@H]1C[C@H](NC(=O)c2ncc(Cl)s2)C[C@H](n2c(-c3ccccc3F)nc3cnc(-n4cncn4)cc32)C1] | (400 MHz, CDCl₃) δ 9.28 (s, 1 H), 8.97 (s, 1 H), 8.17 (s, 1 H), 8.08 (s, 1 H), 7.69-7.64 (m, 3 H), 7.44-7.34 (m, 2 H), 7.10- 7.08 (m, 1 H), 4.70-4.68 (m, 1 H), 4.40-4.36 (m, 1 H), 3.87 (s, 1 H), 3.29 (s, 3 H), 2.43-2.27 (m, 5 H), 1.61-1.57 (m, 1 H). | m/z 553.2 [M + H]⁺; (column: REGIS(S,S)WHELK- O1 (250 mm*25 mm, 10 um); mobile phase: [0.1% NH₃H₂O EtOH]; B %: 50%-50%, min). Rt = 3.527 min 98.4% ee |
| 417 | 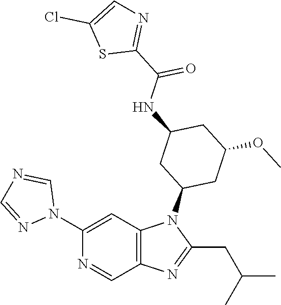 [SMILES: CO[C@H]1C[C@H](NC(=O)c2ncc(Cl)s2)C[C@H](n2c(CC(C)C)nc3cnc(-n4cncn4)cc32)C1] | (400 MHz, CDCl₃) δ 9.23 (s, 1 H), 8.84 (s, 1 H), 8.13 (s, 1 H), 7.94 (s, 1 H), 7.65 (s, 1 H), 7.16-7.14 (m, 1 H), 4.85 (br s, 1 H), 4.56-4.54 (m, 1 H), 3.92 (s, 1 H), 3.44 (s, 3 H), 2.87- 2.85 (m, 2 H), 2.53-2.27 (m, 6 H), 1.67-1.60 (m, 1 H), 1.12- 1.08 (m, 6 H). | m/z 515.2 [M + H]⁺; (column: Phenomenex- Cellulose-2 (250 mm*30 mm, 10 um); mobile phase: [0.1% NH₃H₂O MeOH]; B %: 60%-60%, min). Rt = 1.759 min 100% ee |
| 418 | 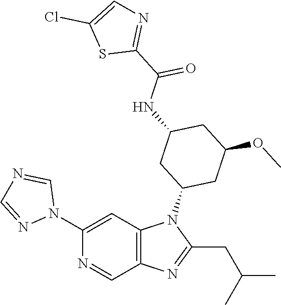 [SMILES: CO[C@@H]1C[C@@H](NC(=O)c2ncc(Cl)s2)C[C@@H](n2c(CC(C)C)nc3cnc(-n4cncn4)cc32)C1] | (400 MHz, CDCl₃) δ 9.23 (s, 1 H), 8.84 (s, 1 H), 8.13 (s, 1 H), 7.94 (s, 1 H), 7.65 (s, 1 H), 7.16-7.14 (m, 1 H), 4.86 (br s, 1 H), 4.57-4.52 (m, 1 H), 3.92- 3.91 (m, 1 H), 3.44 (s, 3 H), 2.87-2.85 (m, 2 H), 2.53-2.27 (m, 6 H), 1.67-1.60 (m, 1 H), 1.12-1.08 (m, 6 H). | m/z 515.2 [M + H]⁺; (column: Phenomenex- Cellulose-2 (250 mm*30 mm, 10 um); mobile phase: [0.1% NH₃H₂O MeOH]; B %: 60%-60%, min). Rt = 3.288 min 99.8% ee |
| 421 | 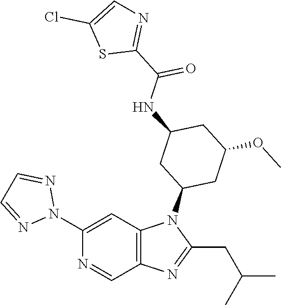 [SMILES: CO[C@H]1C[C@H](NC(=O)c2ncc(Cl)s2)C[C@H](n2c(CC(C)C)nc3cnc(-n4nccn4)cc32)C1] | (400 MHz, CDCl₃) δ 8.98 (br s, 1 H), 8.10 (s, 1 H), 7.92 (s, 2 H), 7.64 (s, 1 H), 7.15-7.12 (m, 1 H), 4.87 (br s, 1 H), 4.56- 4.54 (m, 1 H), 3.92 (br s, 1 H), 3.44 (s, 3 H), 2.88-2.86 (m, 2 H), 2.42-2.19 (m, 6 H), 1.65- 1.59 (m, 1 H), 1.12-1.09 (m, 6 H) | m/z 515.2 [M + H]⁺; (column: DAICEL CHIRALCEL OD (250 mm*30 mm, 10 um); mobile phase: [0.1% NH₃H₂O EtOH]; B %: 30%-30%, min). Rt = 4.293 min 100% ee |
| 422 | 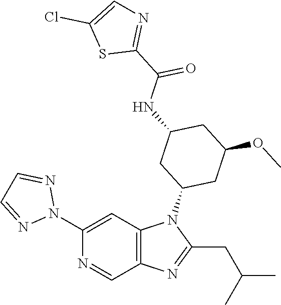 [SMILES: CO[C@@H]1C[C@@H](NC(=O)c2ncc(Cl)s2)C[C@@H](n2c(CC(C)C)nc3cnc(-n4nccn4)cc32)C1] | (400 MHz, CDCl₃) δ 8.98 (s, 1 H), 8.10 (br s, 1 H), 7.93 (s, 2 H), 7.64 (s, 1 H), 7.15-7.13 (m, 1 H), 4.89-4.84 (m, 1 H), 4.56-4.54 (m, 1 H), 3.92 (br s, 1 H), 3.44 (s, 3 H), 2.88-2.86 (m, 2 H), 2.42-2.19 (m, 6 H), 1.65-1.59 (m, 1 H), 1.12-1.09 (m, 6 H). | m/z 515.2 [M + H]⁺; (column: DAICEL CHIRALCEL OD (250 mm*30 mm, 10 um); mobile phase: [0.1% NH₃H₂O EtOH]; B %: 30%-30%, min). Rt = 4.918 min 100% ee |
| 423 | 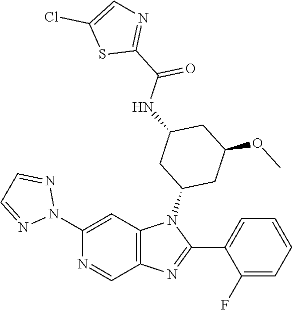 [SMILES: CO[C@@H]1C[C@@H](NC(=O)c2ncc(Cl)s2)C[C@@H](n2c(-c3ccccc3F)nc3cnc(-n4nccn4)cc32)C1] | (400 MHz, CDCl₃) δ 9.09 (s, 1 H), 8.22 (s, 1 H), 7.95 (s, 2 H), 7.66-7.62 (m, 3 H), 7.42-7.29 (m, 2 H), 7.08-7.06 (m, 1 H), 4.69 (br s, 1 H), 4.39-4.37 (m, 1 H), 3.86 (s, 1 H), 3.30 (s, 3 H), 2.43-2.28 (m, 5 H), 1.62- 1.58 (m, 1 H). | m/z 553.2 [M + H]⁺; (column: DAICEL CHIRALPAK AD (250 mm*30 mm, 10 um); mobile phase: [0.1% NH₃H₂O MeOH]; B %: 40%-40%, min). Rt = 1.595 min 99.6% ee |
| 424 | 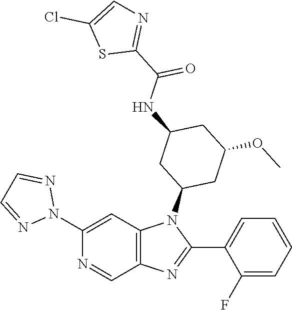 [SMILES: CO[C@H]1C[C@H](NC(=O)c2ncc(Cl)s2)C[C@H](n2c(-c3ccccc3F)nc3cnc(-n4nccn4)cc32)C1] | (400 MHz, CDCl₃) δ 9.09 (s, 1 H), 8.23 (s, 1 H), 7.95 (s, 2 H), 7.66-7.62 (m, 3 H), 7.42-7.29 (m, 2 H), 7.08-7.06 (m, 1 H), 4.69 (br s, 1 H), 4.39-4.37 (m, 1 H), 3.87 (s, 1 H), 3.30 (s, 3 H), 2.43-2.29 (m, 5 H), 1.62- 1.58 (m, 1 H). | m/z 553.1 [M + H]⁺; (column: DAICEL CHIRALPAK AD (250 mm*30 mm, 10 um); mobile phase: [0.1% NH₃H₂O MeOH]; B%: 40%-40%, min). Rt = 2.126 min 99.4% ee |
| 429 | 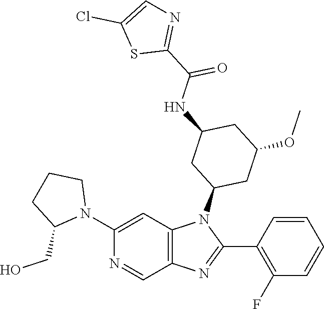 [SMILES: CO[C@H]1C[C@H](NC(=O)c2ncc(Cl)s2)C[C@H](n2c(-c3ccccc3F)nc3cnc(N4CCC[C@H]4CO)cc32)C1] | (400 MHz, CDCl₃) δ 8.55 (s, 1 H), 7.55-7.50 (m, 3 H), 7.29- 7.25 (m, 1 H), 7.19-7.13 (m, 1 H), 7.04-7.03 (m, 1 H), 6.35 (s, 1 H), 4.49-4.36 (m, 2 H), 4.21 (br s, 1 H), 3.79-3.50 (m, 4 H), 3.34-3.26 (m, 1 H), 3.20 (s, 3 H), 2.35-1.97 (m, 8 H), 1.50- 1.32 (m, 1 H). | m/z 585.2 [M + H]⁺; (column: DAICEL CHIRALPAK AS (250 mm*30 mm, 10 um); mobile phase: [0.1% NH₃H₂O EtOH]; B %: 25%-25%, min). Rt = 2.907 min 100% ee |
| 430 | 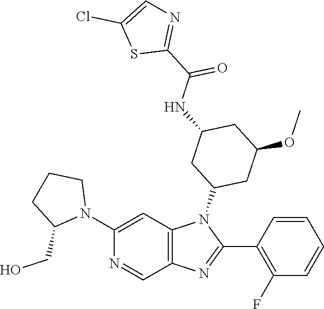 | (400 MHz, CDCl₃) δ 8.56 (s, 1 H), 7.55 (s, 1 H), 7.53-7.44 (m, 2 H), 7.29-7.25 (t, 1 H), 7.17 (br s, 1 H), 7.04-7.03 (m, 1 H), 6.36 (s, 1 H), 4.53-4.32 (m, 2 H), 4.21-4.19 (m, 1 H), 3.78- 3.57 (m, 3 H), 3.52 (br s, 1 H), 3.39-3.26 (m, 1 H), 3.19 (s, 3 H), 2.31-1.99 (m, 8 H), 1.46 (br s, 1 H). | m/z 585.2 [M + H]⁺; (column: DAICEL CHIRALPAK AS (250 mm*30 mm, 10 um); mobile phase: [0.1% NH₃H₂O EtOH]; B %: 25%-25%, min). Rt = 3.561 min 98.2% ee |
| 439 | 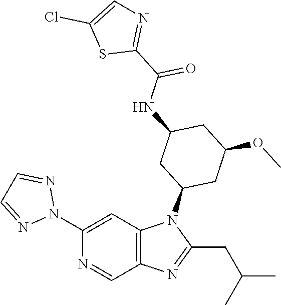 [SMILES: CO[C@@H]1C[C@H](NC(=O)c2ncc(Cl)s2)C[C@H](n2c(CC(C)C)nc3cnc(-n4nccn4)cc32)C1] | (400 MHz, CDCl₃) δ 8.97 (s, 1 H), 8.14 (s, 1 H), 7.93 (s, 2 H), 7.67 (s, 1 H), 7.27 (br s, 1 H), 4.46-4.40 (m, 1 H), 4.33-4.17 (m, 1 H), 3.61-3.49 (m, 1 H), 3.45 (s, 3 H), 2.85-2.83 (m, 2 H), 2.64-2.62 (m, 1 H), 2.45- 2.22 (m, 5 H), 1.60-1.51 (m, 1 H), 1.12-1.10 (m, 6 H). | m/z 515.2 [M + H]⁺; (column: DAICEL CHIRALCEL OD-H (250 mm*30 mm, 5 um); mobile phase: [0.1% NH₃H₂O EtOH]; B %: 35%-35%, min). Rt = 4.641 min 100% ee |
| 440 | 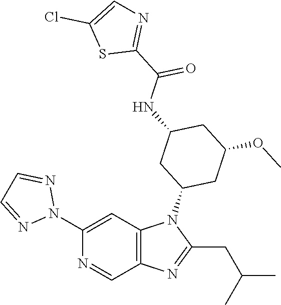 [SMILES: CO[C@H]1C[C@@H](NC(=O)c2ncc(Cl)s2)C[C@@H](n2c(CC(C)C)nc3cnc(-n4nccn4)cc32)C1] | (400 MHz, CDCl₃) δ 8.97 (s, 1 H), 8.14 (s, 1 H), 7.93 (s, 2 H), 7.67 (s, 1 H), 7.27 (br s, 1 H), 4.46-4.40 (m, 1 H), 4.33-4.17 (m, 1 H), 3.61-3.49 (m, 1 H), 3.45 (s, 3 H), 2.85-2.83 (m, 2 H), 2.64-2.62 (m, 1 H), 2.45- 2.22 (m, 5 H), 1.60-1.52 (m, 1 H), 1.11-1.09 (m, 6 H). | m/z 515.2 [M + H]⁺; (column: DAICEL CHIRALCEL OD-H (250 mm*30 mm, 5 um); mobile phase: [0.1% NH₃H₂O EtOH]; B %: 35%-35%, min). Rt = 5.679 min 100% ee |

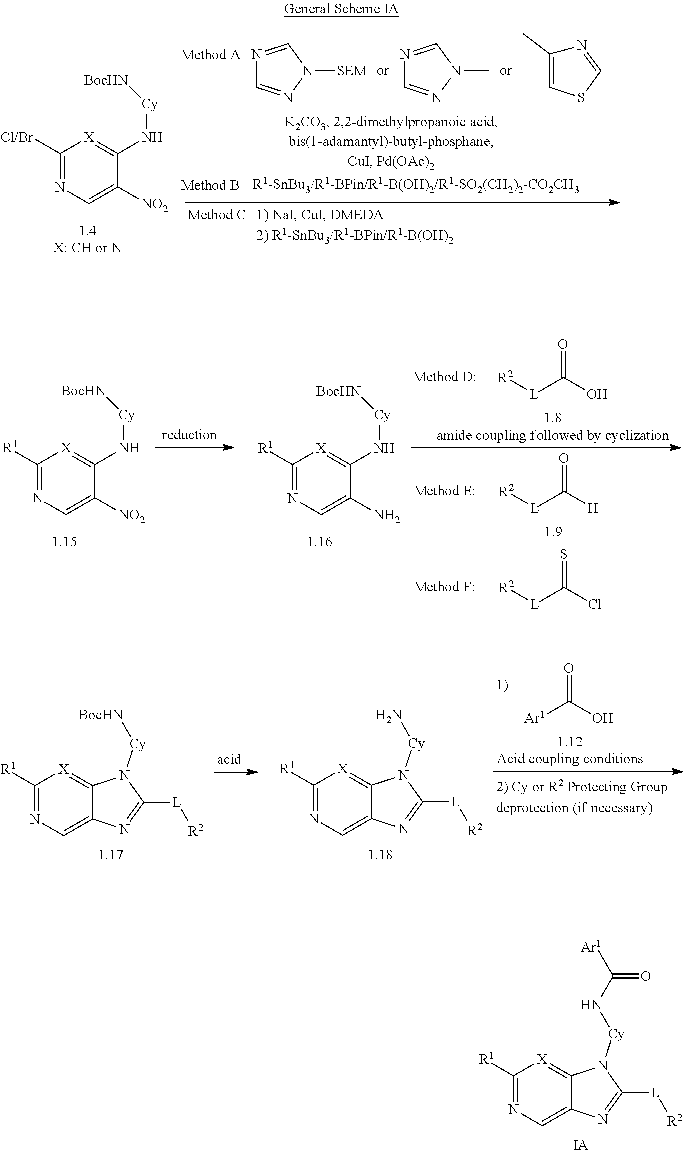

Compounds of formula IA were obtained through General Scheme IA. Beginning with a compound of formula 1.4 (prepared via General Scheme I), the chloride/bromide moiety of a compound of formula 1.4 can be used as a functional handle to install various R¹ aryls, R¹ heteroaryls, and R¹ heterocycloalkyls. Under Method A, unsubstituted and substituted 1,2,4-triazoles, thiazoles, and oxazoles can be installed via a C—H functionalization reaction using, for example, 1-((2-(trimethylsilyl)ethoxy)methyl)-1H-1,2,4-triazole, 1-methyl-1H-1,2,4-triazole, or 4-methylthiazole. Under Method B, the chloride/bromide moiety of a compound of formula 1.4 was converted to a R¹ heteroaryl or aryl using a Stille coupling (R¹—SnBu₃), Suzuki coupling (R¹-BPin or R¹—B(OH)₂), or a cross-coupling with a substituted sulfone (R¹—SO₂(CH₂)₂—CO₂CH₃) to afford compounds of formula 1.15. In some cases, the chloride/bromide moiety needed to be converted to the more reactive iodide moiety using the reaction conditions in Method C prior to the Stille or Suzuki coupling. The nitro group in a compound of formula 1.15 was reduced using a reducing reagent such as iron in the presence of ammonium chloride or Pd/C to afford the pyridine-diamine compounds of formula 1.16. Subsequently, various L and R² groups were installed by coupling the pyridine-diamine compound of formula 1.16 with a L-R² carboxylic acid of formula 1.8 to form an amide intermediate (Method D), which was then cyclized under either basic or acidic conditions to form a compound of formula 1.17. Alternatively, the pyridine-diamine compound of formula 1.16 was condensed with an aldehyde of formula 1.9 (Method E), followed by cyclization and in situ oxidation to afford a compound of formula 1.17. Alternatively, as shown in Method F, the pyridine-diamine compound of formula 1.16 was condensed with a carbothioyl chloride to form an amide intermediate, which was then cyclized under basic or acidic conditions to form a compound of formula 1.17. The Boc protecting group of a compound of formula 1.17 was then removed under acidic conditions such as TFA or HCl to form a compound of formula 1.18. Ar¹ groups were then installed by reacting an Ar¹ carboxylic acid of formula 1.12 or a basic salt (i.e., Li, K, or Na) thereof with the amine of formula 1.18 using acid coupling conditions known in the art, such as using one of the following reagents—HATU, EDCI, HOBt—along with a base, such as DIPEA (Hunig's base), pyridine, or TEA. If the compounds still contain any protecting groups on the Cy or R² groups, they may be removed under acidic conditions such as hydrochloric acid to afford compounds of formula IA.

### Preparation of Examples 218 and 219 Via General Scheme IA (Methods A and E) (relative stereochemistry is indicated for Steps A-F)

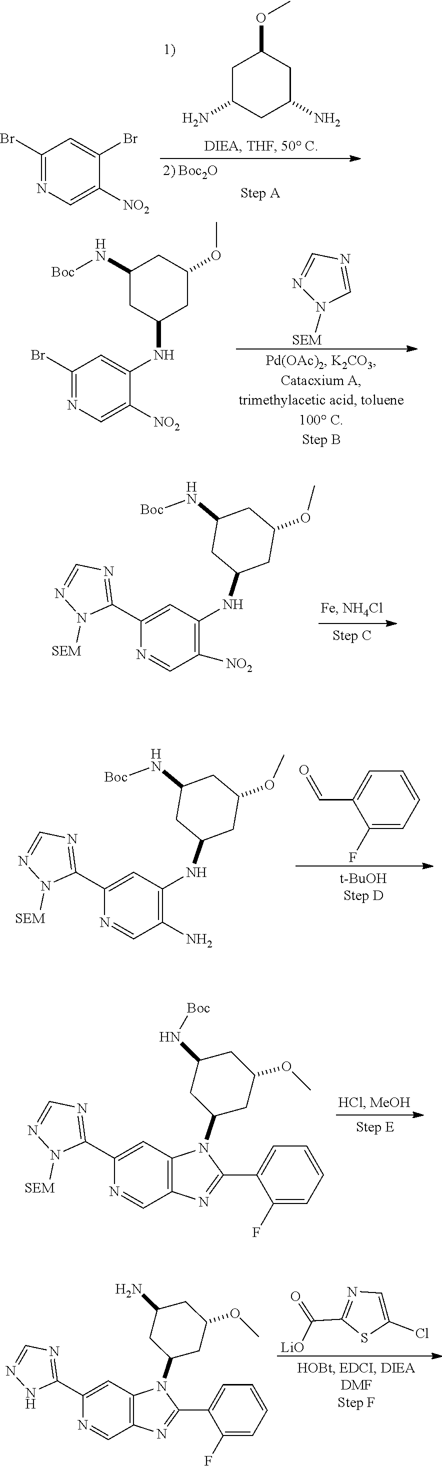

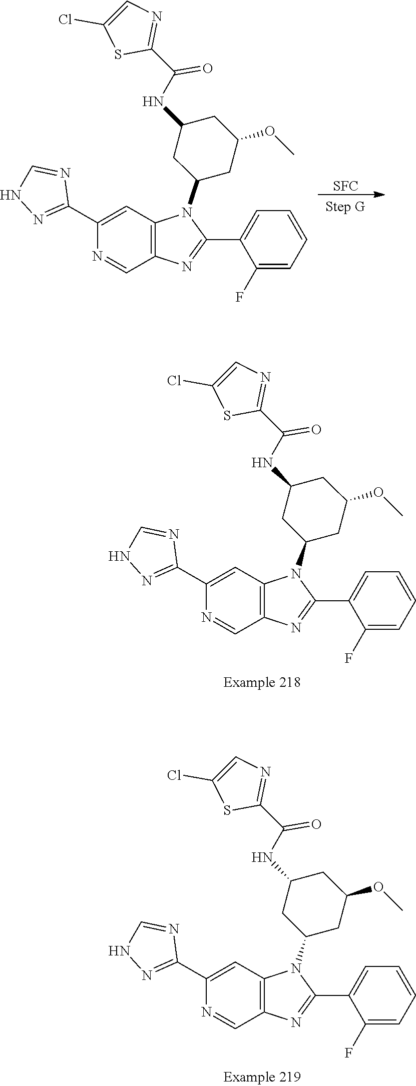

### Step A. Synthesis of tert-butyl (3-((2-bromo-5-nitropyridin-4-yl)amino)-5-methoxycyclohexyl)carbamate

To a solution of 5-methoxycyclohexane-1,3-diamine (80 mg, 555 μmol), prepared in accordance with Intermediate Scheme 2A, in EtOH (2 mL) was added DIEA (215 mg, 1.66 mmol, 290 μL) and 2,4-dibromo-5-nitro-pyridine (78.2 mg, 277 μmol). The mixture was stirred at 40° C. for 2 hrs. The reaction mixture was concentrated to dryness to afford N³-(2-bromo-5-nitro-4-pyridyl)-5-methoxy-cyclohexane-1,3-diamine (150 mg).

To a solution of N³-(2-bromo-5-nitro-4-pyridyl)-5-methoxy-cyclohexane-1,3-diamine (150 mg, 435 μmol) in EtOH (4 mL) was added (Boc)₂O (190 mg, 869 μmol, 200 μL) and DIEA (168 mg, 1.30 mmol, 227 μL). The reaction mixture was stirred at rt for 2 hrs and then concentrated to dryness. The residue was purified by flash silica gel chromatography to give tert-butyl (3-((2-bromo-5-nitropyridin-4-yl)amino)-5-methoxycyclohexyl) carbamate (130 mg, 60.5% yield). LC-MS: m/z 446.7 (M+H)⁺

### Step B. Synthesis of tert-butyl (3-methoxy-5-((5-nitro-2-(1-((2-(trimethylsilyl)ethoxy)methyl)-1H-1,2,4-triazol-5-yl)pyridin-4-yl)amino)cyclohexyl)carbamate

A mixture of tert-butyl (3-((2-bromo-5-nitropyridin-4-yl)amino)-5-methoxycyclohexyl) carbamate (80 mg, 180 μmol), trimethyl-[2-(1,2,4-triazol-1-ylmethoxy)ethyl]silane (71.6 mg, 359 μmol), Pd(OAc)₂ (16.1 mg, 71.9 μmol), K₂CO₃ (74.5 mg, 539 μmol), bis(1-adamantyl)-butyl-phosphane (51.5 mg, 144 μmol), 2,2-dimethylpropanoic acid (18.4 mg, 180 μmol) and CuI (20.5 mg, 108 μmol) in toluene (10 mL) was degassed and purged with N₂ for 3 times, and then the reaction mixture was stirred at 110° C. for 15 hrs under N₂ atmosphere. The reaction mixture was combined with another batch of tert-butyl (3-((2-bromo-5-nitropyridin-4-yl)amino)-5-methoxycyclohexyl) carbamate (450 mg) and concentrated to dryness. The residue was purified by flash silica gel chromatography to give tert-butyl (3-methoxy-5-((5-nitro-2-(1-((2-(trimethylsilyl)ethoxy)methyl)-1H-1,2,4-triazol-5-yl)pyridin-4-yl)amino)cyclohexyl)carbamate (600 mg). LC-MS: m/z 564.3 (M+H)⁺.

### Step C. Synthesis of tert-butyl (3-((5-amino-2-(1-((2-(trimethylsilyl)ethoxy)methyl)-1H-1,2,4-triazol-5-yl)pyridin-4-yl)amino)-5-methoxycyclohexyl)carbamate

To a solution of tert-butyl (3-methoxy-5-((5-nitro-2-(1-((2-(trimethylsilyl)ethoxy)methyl)-1H-1,2,4-triazol-5-yl)pyridin-4-yl)amino)cyclohexyl) carbamate (550 mg, 976 μmol) in THF (4 mL) was added Fe (272 mg, 4.88 mmol), H₂O (1 mL) and NH₄Cl (78.3 mg, 1.46 mmol). The reaction mixture was stirred at 75° C. for 15 hrs. The reaction mixture was filtered and concentrated to dryness to give tert-butyl (3-((5-amino-2-(1-((2-(trimethylsilyl)ethoxy) methyl)-1H-1,2,4-triazol-5-yl)pyridin-4-yl)amino)-5-methoxycyclohexyl)carbamate (520 mg). LC-MS: m/z 534.3 (M+H)⁺.

### Step D. Synthesis of tert-butyl (3-(2-(2-fluorophenyl)-6-(1-((2-(trimethylsilyl)ethoxy)methyl)-1H-1,2,4-triazol-5-yl)-1H-imidazo[4,5-c]pyridin-1-yl)-5-methoxycyclohexyl)carbamate

To a solution of tert-butyl (3-((5-amino-2-(1-((2-(trimethylsilyl)ethoxy)methyl)-1H-1,2,4-triazol-5-yl)pyridin-4-yl)amino)-5-methoxy cyclohexyl)carbamate (520 mg, 974 μmol) in t-BuOH (5 mL) was added 2-fluorobenzaldehyde (145 mg, 1.17 mmol). The reaction mixture was stirred at 85° C. for 15 hrs and then concentrated to dryness. The residue was purified by flash silica gel chromatography to give tert-butyl (3-(2-(2-fluorophenyl)-6-(1-((2-(trimethylsilyl)ethoxy)methyl)-1H-1,2,4-triazol-5-yl)-1H-imidazo[4,5-c]pyridin-1-yl)-5-methoxycyclohexyl)carbamate (390 mg, 58.1% yield). LC-MS: m/z 638.3 (M+H)⁺.

### Step E. Synthesis of (3-(2-(2-fluorophenyl)-6-(1H-1,2,4-triazol-5-yl)-1H-imidazo[4,5-c]pyridin-1-yl)-5-methoxycyclohexan-1-amine

To a solution of tert-butyl (3-(2-(2-fluorophenyl)-6-(1-((2-(trimethylsilyl)ethoxy)methyl)-1H-1,2,4-triazol-5-yl)-1H-imidazo[4,5-c]pyridin-1-yl)-5-methoxycyclohexyl)carbamate (370 mg, 580 μmol) in MeOH (10 mL) was added HCl/MeOH (4 M, 9.25 mL). The reaction mixture was stirred at rt for 2 hrs and then concentrated to dryness to give (3-(2-(2-fluorophenyl)-6-(1H-1,2,4-triazol-5-yl)-1H-imidazo[4,5-c]pyridin-1-yl)-5-methoxycyclohexan-1-amine (240 mg), which was used without further purification. LC-MS: m/z 408.2 (M+H)⁺.

### Step F. Synthesis of 5-chloro-N-(3-(2-(2-fluorophenyl)-6-(1H-1,2,4-triazol-3-yl)-1H-imidazo[4,5-c]pyridin-1-yl)-5-methoxycyclohexyl)thiazole-2-carboxamide

To (3-(2-(2-fluorophenyl)-6-(1H-1,2,4-triazol-5-yl)-1H-imidazo[4,5-c]pyridin-1-yl)-5-methoxycyclohexan-1-amine (240 mg, 589 μmol), (5-chlorothiazole-2-carbonyl)oxylithium (120 mg, 707 μmol) in DMF (5 mL) was added, followed by HOBt (119 mg, 884 μmol), EDCI (169 mg, 884 μmol) and DIEA (228 mg, 1.77 mmol), and then the reaction mixture was stirred at rt for 15 hrs. The reaction mixture was concentrated to dryness. The residue was purified by prep-HPLC (column: Welch Xtimate C18 150*30 mm*5 um; mobile phase: [water (FA)-ACN]; B %: 30%-60%, 7 min) to give 5-chloro-N-(3-(2-(2-fluorophenyl)-6-(1H-1,2,4-triazol-3-yl)-1H-imidazo[4,5-c]pyridin-1-yl)-5-methoxycyclohexyl)thiazole-2-carboxamide (80 mg, 23.9% yield). LC-MS: m/z 553.1 (M+H)⁺.

### Step G. Synthesis of 5-chloro-N-((1R,3S,5S)-3-(2-(2-fluorophenyl)-6-(1H-1,2,4-triazol-3-yl)-1H-imidazo[4,5-c]pyridin-1-yl)-5-methoxycyclohexyl)thiazole-2-carboxamide (Example 218) and 5-chloro-N-((1S,3R,5R)-3-(2-(2-fluorophenyl)-6-(1H-1,2,4-triazol-3-yl)-1H-imidazo[4,5-c]pyridin-1-yl)-5-methoxycyclohexyl)thiazole-2-carboxamide Example 219

The enantiomers of 5-chloro-N-(3-(2-(2-fluorophenyl)-6-(1H-1,2,4-triazol-3-yl)-1H-imidazo[4,5-c]pyridin-1-yl)-5-methoxycyclohexyl)thiazole-2-carboxamide (80 mg, 145 μmol) were separated by SFC (column: DAICEL CHIRALPAK AD (250 mm*30 mm, 10 um); mobile phase: [0.1% NH₃H₂O IPA]; %: 30%-30%,min) to give Enantiomer #1 (18.6 mg, 22.5% yield, Rt=1.038 min) and Enantiomer #2 (18.5 mg, 22.4% yield, Rt=1.453 min).

Enantiomer #1: 5-chloro-N-((1S,3R,5R)-3-(2-(2-fluorophenyl)-6-(1H-1,2,4-triazol-3-yl)-1H-imidazo[4,5-c]pyridin-1-yl)-5-methoxycyclohexyl)thiazole-2-carboxamide (Example 219). ¹H NMR (400 MHz, CD₃OD) δ 9.11 (s, 1H), 8.66 (s, 1H), 8.49 (s, 1H), 8.37 (br s, 1H), 7.81-7.73 (m, 3H), 7.52-7.46 (m, 2H), 4.69-4.63 (m, 1H), 4.34-4.28 (m, 1H), 3.90 (br s, 1H), 3.29 (s, 3H), 2.58-2.53 (m, 2H), 2.36-2.28 (m, 3H), 1.87-1.84 (m, 1H); LC-MS: m/z 553.2 (M+H)⁺; 100% ee.

Enantiomer #2: 5-chloro-N-((1R,3S,5S)-3-(2-(2-fluorophenyl)-6-(1H-1,2,4-triazol-3-yl)-1H-imidazo[4,5-c]pyridin-1-yl)-5-methoxycyclohexyl)thiazole-2-carboxamide (Example 218). ¹H NMR (400 MHz, CD₃OD) δ 9.11 (s, 1H), 8.66 (s, 1H), 8.49 (s, 1H), 8.37 (br s, 1H), 7.82-7.74 (m, 3H), 7.51-7.46 (m, 2H), 4.68-4.62 (m, 1H), 4.34-4.28 (m, 1H), 3.90 (br s, 1H), 3.29 (s, 3H), 2.61-2.50 (m, 2H), 2.36-2.26 (m, 3H), 1.87-1.80 (m, 1H); LC-MS: m/z 553.2 (M+H)⁺; 97.0% ee.

### Preparation of Example 336 Via General Scheme IA (Methods B and E)

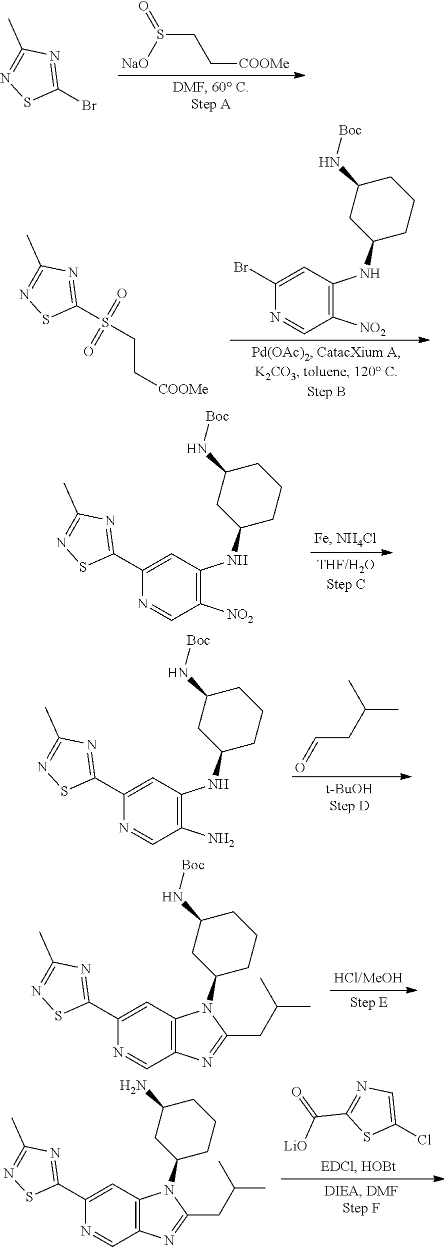

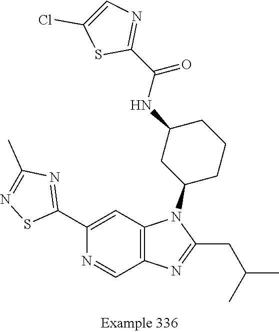 [SMILES: Cc1nsc(-c2cc3c(cn2)nc(CC(C)C)n3[C@@H]2CCC[C@H](NC(=O)c3ncc(Cl)s3)C2)n1]

### Step A. Synthesis of methyl 3-((3-methyl-1,2,4-thiadiazol-5-yl)sulfonyl)propanoate

To a solution of 5-bromo-3-methyl-1,2,4-thiadiazole (300 mg, 1.68 mmol) in DMF (4 mL) was added sodium 3-methoxy-3-oxo-propane-1-sulfinate (379 mg, 2.18 mmol). The reaction mixture was stirred at 60° C. for 12 hrs and then allowed to cool. Water (5 mL) was added, followed by an additional 15 mL of water. The aqueous portion was extracted with EtOAc (20 mL×3). The combined organic layers were washed with brine (20 mL×3), dried over Na₂SO₄, filtered, and concentrated to dryness. The residue was purified by column chromatography to give methyl 3-((3-methyl-1,2,4-thiadiazol-5-yl)sulfonyl)propanoate (198 mg, 44.9% yield). LC-MS: m/z 251.0 [M+H]⁺.

### Step B. Synthesis of tert-butyl ((1S,3R)-3-((2-(3-methyl-1,2,4-thiadiazol-5-yl)-5-nitropyridin-4-yl)amino)cyclohexyl)carbamate

A mixture of methyl 3-[(3-methyl-1,2,4-thiadiazol-5-yl)sulfonyl]propanoate (198 mg, 791 μmol), tert-butyl N-[(1S,3R)-3-[(2-bromo-5-nitro-4-pyridyl)amino]cyclohexyl]carbamate (329 mg, 791 μmol), Pd(OAc)₂ (35.5 mg, 158 μmol), bis(1-adamantyl)-butyl-phosphane (113 mg, 316 μmol) and K₂CO₃ (273 mg, 1.98 mmol) in toluene (3 mL) was degassed and purged with N₂ for 3 times, and then the reaction mixture was stirred at 120° C. for 12 hrs under N₂ atmosphere. Water (20 mL) was added, and the aqueous portion was extracted with EtOAc (20 mL×3). The combined organic layers were washed with brine (20 mL×3), dried over Na₂SO₄, filtered, and concentrated to dryness. The residue was purified by column chromatography to give tert-butyl ((1S,3R)-3-((2-(3-methyl-1,2,4-thiadiazol-5-yl)-5-nitropyridin-4-yl)amino)cyclohexyl)carbamate (34 mg, 9.9% yield). LC-MS: m/z 435.2 [M+H]⁺.

### Step C. Synthesis of tert-butyl ((1S,3R)-3-((5-amino-2-(3-methyl-1,2,4-thiadiazol-5-yl)pyridin-4-yl)amino)cyclohexyl)carbamate

To a solution of tert-butyl ((1S,3R)-3-((2-(3-methyl-1,2,4-thiadiazol-5-yl)-5-nitropyridin-4-yl)amino)cyclohexyl)carbamate (34 mg, 78.3 μmol) in THF (3 mL) and H₂O (1.5 mL) was added Fe (21.9 mg, 391 μmol) and NH₄Cl (20.9 mg, 391 μmol). The reaction mixture was stirred at 70° C. for 12 hrs. The reaction mixture was allowed to cool and was then filtered. The cake was washed with THF (10 mL×3). The combined organic layers were concentrated to dryness to give tert-butyl ((1S,3R)-3-((5-amino-2-(3-methyl-1,2,4-thiadiazol-5-yl)pyridin-4-yl)amino)cyclohexyl)carbamate (31 mg), which was used without further purification. LC-MS: m/z 405.3 [M+H]⁺.

### Step D. Synthesis of tert-butyl ((1S,3R)-3-(2-isobutyl-6-(3-methyl-1,2,4-thiadiazol-5-yl)-1H-imidazo[4,5-c]pyridin-1-yl)cyclohexyl)carbamate

To a solution of tert-butyl ((1S,3R)-3-((5-amino-2-(3-methyl-1,2,4-thiadiazol-5-yl)pyridin-4-yl)amino)cyclohexyl)carbamate (40 mg, 98.9 μmol) in t-BuOH (1 mL) was added 3-methylbutanal (17.0 mg, 198 μmol). The reaction mixture was stirred at 90° C. for 16 hrs and then concentrated to dryness. The residue was purified by column chromatography to give tert-butyl ((1S,3R)-3-(2-isobutyl-6-(3-methyl-1,2,4-thiadiazol-5-yl)-1H-imidazo[4,5-c]pyridin-1-yl)cyclohexyl)carbamate (42 mg, 81.2% yield). LC-MS: m/z 471.2 [M+H]⁺.

### Step E. Synthesis of (1S,3R)-3-(2-isobutyl-6-(3-methyl-1,2,4-thiadiazol-5-yl)-1H-imidazo[4,5-c]pyridin-1-yl)cyclohexan-1-amine

To a solution of tert-butyl ((1S,3R)-3-(2-isobutyl-6-(3-methyl-1,2,4-thiadiazol-5-yl)-1H-imidazo[4,5-c]pyridin-1-yl)cyclohexyl)carbamate (40 mg, 85.0 μmol) in MeOH (0.5 mL) was added HCl/MeOH (4 M, 2 mL). The reaction mixture was stirred at rt for 2 hrs. The reaction mixture was concentrated to dryness to give (1S,3R)-3-(2-isobutyl-6-(3-methyl-1,2,4-thiadiazol-5-yl)-1H-imidazo[4,5-c]pyridine-1-yl)cyclohexan-1-amine (31 mg), which was used without further purification. LC-MS: m/z 371.2 [M+H]⁺.

### Step F. Synthesis of 5-chloro-N-((1S,3R)-3-(2-isobutyl-6-(3-methyl-1,2,4-thiadiazol-5-yl)-1H-imidazo[4,5-c]pyridin-1-yl)cyclohexyl)thiazole-2-carboxamide (Example 336)

To a solution of (1S,3R)-3-(2-isobutyl-6-(3-methyl-1,2,4-thiadiazol-5-yl)-1H-imidazo[4,5-c]pyridine-1-yl)cyclohexan-1-amine (31 mg, 83.7 μmol) in DMF (2 mL) was added EDCI (24.1 mg, 126 μmol) and HOBt (17.0 mg, 126 μmol), DIEA (32.4 mg, 251 μmol, 43.7 μL) and (5-chlorothiazole-2-carbonyl)oxylithium (21.3 mg, 126 μmol). The reaction mixture was stirred at rt for 12 hrs. Water (20 mL) was added, and the aqueous portion extracted with EtOAc (20 mL×3). The combined organic layers were washed with brine (20 mL×3), dried over Na₂SO₄, filtered, and concentrated to dryness. The residue was purified by column chromatography, and then further purified by reversed-phase HPLC (column: Welch Xtimate C18 150*30 mm*5 um; mobile phase: [water(FA)-ACN]; B %: 55%-85%, 7 min) to give Example 336 (16.4 mg, 37.7% yield). ¹H NMR (400 MHz, CDCl₃) δ 9.03 (s, 1H), 8.31 (s, 1H), 7.65 (s, 1H), 7.24-7.22 (m, 1H), 4.45-4.38 (m, 1H), 4.22-4.20 (m, 1H), 2.86-2.84 (m, 2H), 2.78 (s, 3H), 2.36-2.14 (m, 6H), 2.02-1.99 (m, 1H), 1.60-1.58 (m, 2H), 1.10-1.08 (m, 6H); LC-MS: m/z 516.2 [M+H]⁺.

General Scheme IA set forth above was used to synthesize the following compounds by using appropriate starting materials:

| Ex. | | | |
|---|---|---|---|
| No. | Structure | ¹H NMR | LC-MS and % ee |
| 159 | 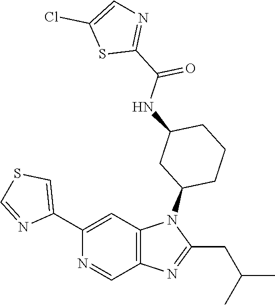 [SMILES: CC(C)Cc1nc2cnc(-c3cscn3)cc2n1[C@@H]1CCC[C@H](NC(=O)c2ncc(Cl)s2)C1] | (400 MHz, CD₃OD) δ 9.16 (s, 1H), 8.86 (s, 1H), 8.49 (s, 1H), 8.22 (s, 1H), 7.79 (s, 1H), 4.67-4.53 (m, 1H), 4.27-4.15 (m, 1H), 3.02-2.88 (m, 2H), 2.62-2.47 (m, 1H), 2.44-2.30 (m, 1H), 2.30-2.18 (m, 2H), 2.15-1.94 (m, 3H), 1.78-1.66 (m, 2H), 1.07 (d, 6H). | m/z 501.2 [M + H]⁺ |
| 162 | 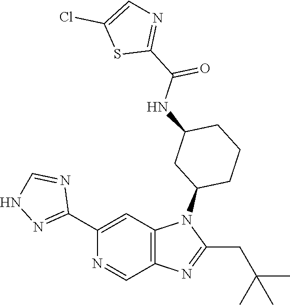 [SMILES: CC(C)(C)Cc1nc2cnc(-c3nc[nH]n3)cc2n1[C@@H]1CCC[C@H](NC(=O)c2ncc(Cl)s2)C1] | (400 MHz, CDCl₃) δ 9.11 (s, 1 H), 8.40 (s, 1 H), 8.15 (s, 1 H), 7.66 (s, 1 H), 7.29-7.27 (m, 1 H), 4.51-4.45 (m, 1 H), 4.21 (br s, 1 H), 2.90 (s, 2 H), 2.39-2.33 (m, 3 H), 2.22-1.99 (m, 3 H), 1.64- 1.61 (m, 2 H), 1.14 (s, 9 H). | m/z 499.2 [M + H]⁺ |
| 164 | 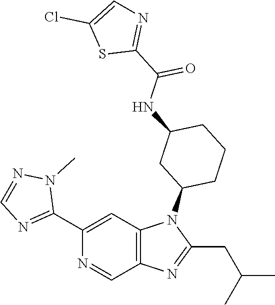 [SMILES: CC(C)Cc1nc2cnc(-c3ncnn3C)cc2n1[C@@H]1CCC[C@H](NC(=O)c2ncc(Cl)s2)C1] | (400 MHz, DMSO-d₆) δ 9.12-9.10 (m, 1 H), 8.99 (s, 1 H), 8.38 (s, 1 H), 8.05-8.03 (m, 2 H), 4.70-4.64 (m, 1 H), 4.28 (s, 3 H), 4.21-4.19 (m, 1 H), 2.92-2.90 (m, 2 H), 2.57- 2.54 (m, 1 H), 2.25-2.20 (m, 1 H), 2.06-1.96 (m, 2 H), 1.88 (br s, 3 H), 1.68-1.58 (m, 2 H), 1.02-1.00 (m, 6 H) | m/z 499.2 [M + H]⁺ |
| 166 | 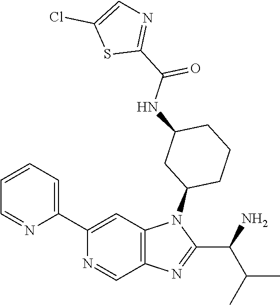 [SMILES: CC(C)[C@H](N)c1nc2cnc(-c3ccccn3)cc2n1[C@@H]1CCC[C@H](NC(=O)c2ncc(Cl)s2)C1] | (400 MHz, DMSO-d₆) δ 9.17 (d, 1H), 8.97 (s, 1H), 8.73 (d, 1H), 8.67 (s, 1H), 8.45 (d, 1H), 8.04 (s, 1H), 7.97-7.90 (m, 1H), 7.45-7.38 (m, 1H), 4.92-4.80 (m, 1H), 4.29-4.14 (m, 1H), 4.02 (d, 1H), 2.64- 2.56 (m, 1H), 2.22-2.04 (m, 3H), 2.00-1.83 (m, 3H), 1.70-1.57 (m, 2H), 1.03 (d, 3H), 0.87 (d, 3H). | m/z 510.2 [M + H]⁺ |
| 167 | 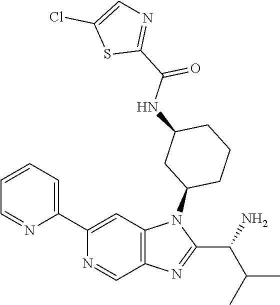 [SMILES: CC(C)[C@@H](N)c1nc2cnc(-c3ccccn3)cc2n1[C@@H]1CCC[C@H](NC(=O)c2ncc(Cl)s2)C1] | (400 MHz, DMSO-d₆) δ 9.18 (d, 1H), 8.99 (s, 1H), 8.74 (d, 1H), 8.68 (s, 1H), 8.45 (d, 1H), 8.21 (s, 1H), 8.05 (s, 1H), 7.97-7.91 (m, 1H), 7.45-7.39 (m, 1H), 4.95-4.80 (m, 1H), 4.24-4.07 (m, 2H), 2.69-2.55 (m, 1H), 2.18-1.84 (m, 7H), 1.71-1.58 (m, 2H), 1.04 (d, 3H), 0.88 (d, 3H). | m/z 510.2 [M + H]⁺ |
| 174 | 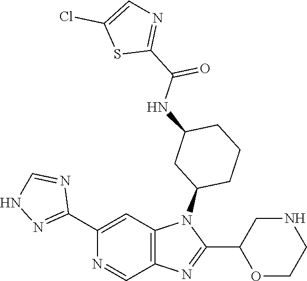 [SMILES: O=C(N[C@H]1CCC[C@@H](n2c(C3CNCCO3)nc3cnc(-c4nc[nH]n4)cc32)C1)c1ncc(Cl)s1] | (400 MHz, DMSO-d₆) δ 9.19-9.05 (m, 2H), 8.42 (s, 1H), 8.15-8.02 (m, 2H), 7.78-7.71 (m, 1H), 5.60-5.50 (m, 1H), 5.39-5.29 (m, 1H), 5.20-5.10 (m, 1H), 4.81-4.66 (m, 1H), 4.28-4.15 (m, 1H), 4.05-3.89 (m, 2H), 3.54-3.51 (m, 3H), 2.08-1.92 (m, 6H), 1.64-1.49 (m, 2H). | m/z 514.0 [M + H]⁺ |
| 208 | 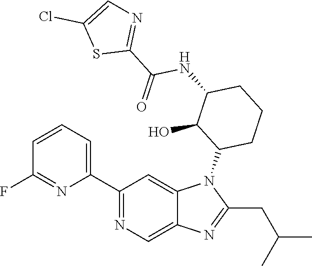 | (400 MHz, CDCl₃) δ 8.88 (s, 1 H), 8.34 (br s, 2 H), 7.94- 7.91 (m, 1 H), 7.62 (s, 1 H), 7.46-7.44 (m, 1 H), 6.90- 6.88 (m, 1 H), 4.39-4.20 (m, 4 H), 2.89-2.87 (m, 2 H), 2.47-2.43 (m, 1 H), 2.34- 2.24 (m, 2 H), 2.08-1.99 (m, 2 H), 2.31 (s, 3 H), 2.06-2.04 (m, 2 H), 1.67-1.64 (m, 2 H), 1.09-1.05 (m, 6 H). | m/z 529.1 [M + H]⁺ (column: DAICEL CHIRALPAK AS (250 mm * 30 mm, 10 um); mobile phase: [0.1% NH₃H₂O EtOH]; B%: 20%-20%, min) Rt = 3.179 min 100% ee |
| 209 | 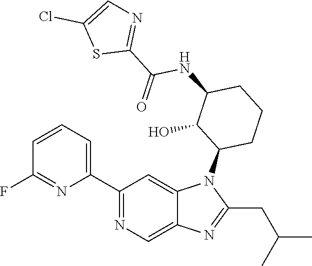 [SMILES: CC(C)Cc1nc2cnc(-c3cccc(F)n3)cc2n1[C@@H]1CCC[C@H](NC(=O)c2ncc(Cl)s2)[C@H]1O] | (400 MHz, CDCl₃) δ 8.88 (s, 1 H), 8.34 (br s, 2 H), 7.94- 7.92 (m, 1 H), 7.62 (s, 1 H), 7.47-7.45 (m, 1 H), 6.90- 6.88 (m, 1 H), 4.40-4.20 (m, 4 H), 2.89-2.87 (m, 2 H), 2.47-2.43 (m, 1 H), 2.34- 2.24 (m, 2 H), 2.08-1.99 (m, 2 H), 2.31 (s, 3 H), 2.06-2.04 (m, 2 H), 1.67-1.64 (m, 2 H), 1.09-1.05 (m, 6 H). | m/z 529.2 [M + H]⁺ (column: DAICEL CHIRALPAK AS (250 mm * 30 mm, 10 um); mobile phase: [0.1% NH₃H₂O EtOH]; B%: 20%-20%, min) Rt = 2.637 min 100% ee |
| 214 | 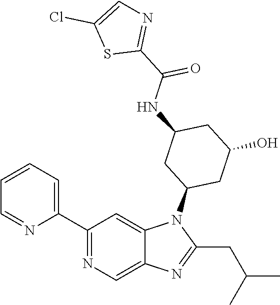 [SMILES: CC(C)Cc1nc2cnc(-c3ccccn3)cc2n1[C@@H]1C[C@@H](O)C[C@H](NC(=O)c2ncc(Cl)s2)C1] | (400 MHz, CDCl₃) δ 9.08 (s, 1 H), 8.69-8.68 (m, 1 H), 8.55-8.49 (m, 2 H), 7.86- 7.82 (m, 1 H), 7.64 (s, 1 H), 7.32-7.29 (m, 1 H), 7.23- 7.21 (m, 1 H), 5.03-4.97 (m, 1 H), 4.73-4.71 (m, 1 H), 4.57 (br s, 1 H), 2.88-2.86 (m, 2 H), 2.58-2.55 (m, 1 H), 2.49-2.29 (m, 4 H), 2.13- 2.10 (m, 2 H), 1.89-1.85 (m, 1 H), 1.10-1.07 (m, 6 H). | m/z 511.0 [M + H]⁺ (column: (s,s) WHELK-O1 (250 mm * 30 mm, 5 um); mobile phase: [0.1% NH₃H₂O IPA]; B%: 50%-50%, min) Rt = 3.945 min 100% ee |
| 215 | 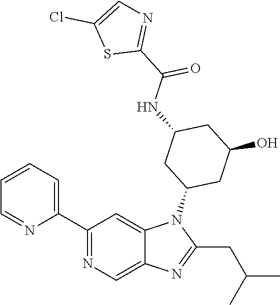 [SMILES: CC(C)Cc1nc2cnc(-c3ccccn3)cc2n1[C@H]1C[C@H](O)C[C@@H](NC(=O)c2ncc(Cl)s2)C1] | (400 MHz, CDCl₃) δ 9.08 (s, 1 H), 8.69-8.68 (m, 1 H), 8.55-8.49 (m, 2 H), 7.86- 7.82 (m, 1 H), 7.64 (s, 1 H), 7.32-7.29 (m, 1 H), 7.23- 7.21 (m, 1 H), 5.03-4.97 (m, 1 H), 4.73-4.71 (m, 1 H), 4.57 (br s, 1 H), 2.88-2.86 (m, 2 H), 2.58-2.55 (m, 1 H), 2.49-2.29 (m, 4 H), 2.13- 2.10 (m, 2 H), 1.89-1.85 (m, 1 H), 1.10-1.07 (m, 6 H). | m/z 511.0 [M + H]⁺ (column: (s,s) WHELK-O1 (250 mm * 30 mm, 5 um); mobile phase: [0.1% NH₃H₂O IPA]; B%: 50%-50%, min) Rt = 3.811 min 100% ee |
| 216 | 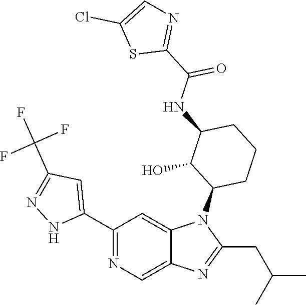 [SMILES: CC(C)Cc1nc2cnc(-c3cc(C(F)(F)F)n[nH]3)cc2n1[C@@H]1CCC[C@H](NC(=O)c2ncc(Cl)s2)[C@H]1O] | (400 MHz, CD₃OD) δ 8.91 (s, 1 H), 8.17 (br s, 1 H), 7.82 (s, 1 H), 7.32 (s, 1 H), 4.85-4.39 (m, 2 H), 4.10- 4.04 (m, 1 H), 3.03-2.97 (m, 1 H), 2.92-2.86 (m, 1 H), 2.54-2.47 (m, 1 H), 2.34- 2.28 (m, 1 H), 2.12-2.10 (m, 1 H), 2.05-1.99 (m, 2 H), 1.90-1.86 (m, 1 H), 1.78- 1.68 (m, 1 H), 1.09-1.04 (m, 6 H). | m/z 568.2 [M + H]⁺ (column: DAICEL CHIRALPAK IG (250 mm * 30 mm, 10 um); mobile phase: [0.1% NH₃H₂O MeOH]; B%: 40%-40%, min). Rt = 1.688 min 98.1% ee |
| 217 | 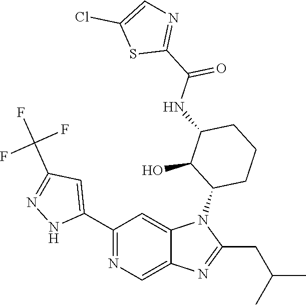 [SMILES: CC(C)Cc1nc2cnc(-c3cc(C(F)(F)F)n[nH]3)cc2n1[C@H]1CCC[C@@H](NC(=O)c2ncc(Cl)s2)[C@@H]1O] | (400 MHz, CDCl₃) δ 8.87 (br s, 1 H), 7.70-7.64 (m, 3 H), 6.82 (br s, 1 H), 4.38 (br s, 1 H), 4.24 (br s, 1 H), 4.16 (br s, 1 H), 2.91-2.77 (m, 2 H), 2.34 (br s, 2 H), 2.15-2.06 (m, 4 H), 1.78-1.64 (m, 2 H), 1.08-0.95 (m, 6 H). | m/z 568.2 [M + H]⁺ (column: DAICEL CHIRALPAK IG (250 mm * 30 mm, 10 um); mobile phase: [0.1% NH₃H₂O MeOH]; B%: 40%-40%, min). Rt = 1.152 min 100% ee |
| 223 | 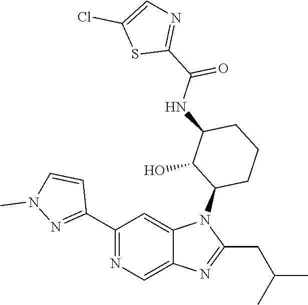 [SMILES: CC(C)Cc1nc2cnc(-c3ccn(C)n3)cc2n1[C@@H]1CCC[C@H](NC(=O)c2ncc(Cl)s2)[C@H]1O] | (400 MHz, CDCl₃) δ 8.76- 8.71 (m, 1 H), 7.84-7.81 (m, 1 H), 7.61-7.60 (m, 1 H), 7.43-7.40 (m, 2 H), 6.85 (br s, 1 H), 4.93 (s, 1H), 4.29- 4.27 (m, 2 H), 4.18-4.11 (m, 1 H), 3.84-3.81 (m, 3 H), 2.87-2.85 (m, 2 H), 2.40 (br s, 1 H), 2.32-2.28 (m, 2 H), 2.00-1.94 (m, 2 H), 1.68- 1.60 (m, 2 H), 1.06-1.04 (m, 6 H). | m/z 514.2 [M + H]⁺ (column: DAICEL CHIRALPAK AD(250 mm * 30 mm, 10 um); mobile phase: [0.1% NH₃H₂O MeOH]; B%: 35%-35%, min) Rt = 1.335 min 100% ee |
| 224 | 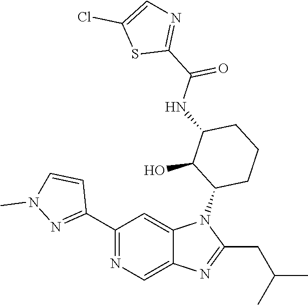 [SMILES: CC(C)Cc1nc2cnc(-c3ccn(C)n3)cc2n1[C@H]1CCC[C@@H](NC(=O)c2ncc(Cl)s2)[C@@H]1O] | (400 MHz, CDCl₃) δ 8.73- 8.71 (m, 1 H), 7.84-7.81 (m, 1 H), 7.61-7.60 (m, 1 H), 7.43-7.40 (m, 2 H), 6.85 (s, 1 H), 4.93 (br s, 1H), 4.29-4.27 (m, 2 H), 4.18-4.11 (m, 1 H), 3.84-3.81 (m, 3 H), 2.87- 2.85 (m, 2 H), 2.40 (br s, 1 H), 2.34-2.30 (m, 2 H), 2.00- 1.94 (m, 2 H), 1.68-1.60 (m, 2 H), 1.05-1.04 (m, 6 H). | m/z 514.2 [M + H]⁺ (column: DAICEL CHIRALPAK AD(250 mm * 30 mm, 10 um); mobile phase: [0.1% NH₃H₂O MeOH]; B%: 35%-35%, min) Rt = 2.292 min 100% ee |
| 225 | 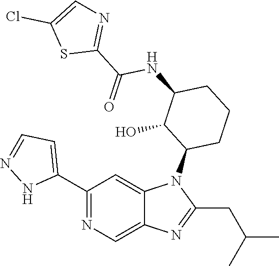 [SMILES: CC(C)Cc1nc2cnc(-c3ccn[nH]3)cc2n1[C@@H]1CCC[C@H](NC(=O)c2ncc(Cl)s2)[C@H]1O] | (400 MHz, CDCl₃) δ 8.03 (br s, 1 H), 7.87 (br s, 2 H), 7.73 (s, 1 H), 7.19 (br s, 1 H), 6.64 (br s, 1 H), 4.21-4.16 (m, 1 H), 4.05 (br s, 1 H), 3.64 (br s, 1 H), 2.98-2.88 (m, 2 H), 2.42-2.31(m, 2 H), 2.11-2.03 (m, 1 H), 1.86- 1.80 (m, 3 H), 1.64-1.55 (m, 3 H), 1.10-1.08 (m, 6 H). | m/z 500.2 [M + H]⁺ (column: DAICEL CHIRALPAK AD(250 mm * 30 mm, 10 um); mobile phase: [0.1% NH₃H₂O EtOH]; B%: 40%-40%, min) Rt = 2.369 min 99.2% ee |
| 226 | 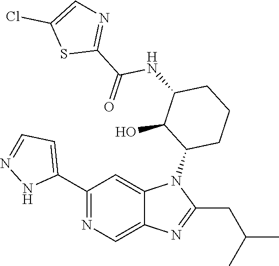 [SMILES: CC(C)Cc1nc2cnc(-c3ccn[nH]3)cc2n1[C@H]1CCC[C@@H](NC(=O)c2ncc(Cl)s2)[C@@H]1O] | (400 MHz, CDCl₃) δ 8.05 (br s, 1 H), 7.86 (br s, 2 H), 7.73 (br s, 1 H), 7.21 (br s, 1 H), 6.65 (br s, 1 H), 4.19 (br s, 1 H), 4.05 (br s, 1 H), 3.64 (br s, 1 H), 2.94-2.92 (m, 2 H), 2.36-2.30 (m, 2 H), 2.06 (br s, 2 H), 1.87-1.81 (m, 3 H), 1.61-1.57 (m, 1 H), 1.09- 1.07 (m, 6 H). | m/z 500.2 [M + H]⁺ (column: DAICEL CHIRALPAK AD(250 mm * 30 mm, 10 um); mobile phase: [0.1% NH₃H₂O EtOH]; B%: 40%-40%, min) Rt = 1.128 min 99.6% ee |
| 229 | 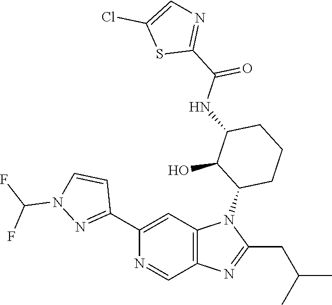 [SMILES: CC(C)Cc1nc2cnc(-c3ccn(C(F)F)n3)cc2n1[C@H]1CCC[C@@H](NC(=O)c2ncc(Cl)s2)[C@@H]1O] | (400 MHz, CDCl₃) δ 8.54 (br s, 1 H), 7.97 (s, 2 H), 7.79 (s, 1 H), 7.55-7.50 (m, 1 H), 7.35 (s, 1 H), 7.20-7.18 (m, 1 H), 4.32-4.15 (m, 3 H), 2.93- 2.91 (m, 2 H), 2.34-2.30 (m, 3 H), 2.04-1.98 (m, 2 H), 1.62-1.53 (m, 1 H), 1.12- 1.00 (m, 7 H). | m/z 550.2 [M + H]⁺ (column: DAICEL CHIRALPAK AS(250 mm * 30 mm, 10 um); mobile phase: [0.1% NH₃H₂O MeOH]; B%: 20%-20%, min) Rt = 2.718 min 100% ee |
| 230 | 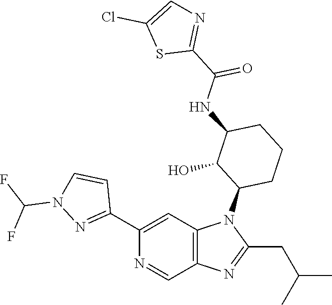 [SMILES: CC(C)Cc1nc2cnc(-c3ccn(C(F)F)n3)cc2n1[C@@H]1CCC[C@H](NC(=O)c2ncc(Cl)s2)[C@H]1O] | (400 MHz, CDCl₃) δ 8.60- 8.52 (m, 1 H), 7.90-7.84 (m, 2 H), 7.69 (s, 1 H), 7.55-7.50 (m, 1 H), 7.35 (s, 1 H), 7.20- 7.18 (m, 1 H), 4.32-4.15 (m, 3 H), 2.93-2.91 (m, 2 H), 2.34-2.30 (m, 3 H), 2.04- 1.98 (m, 2 H), 1.62-1.53 (m, 1 H), 1.12-1.01 (m, 7 H). | m/z 550.2 [M + H]⁺ (column: DAICEL CHIRALPAK AS(250 mm * 30 mm, 10 um); mobile phase: [0.1% NH₃H₂O MeOH]; B%: 20%-20%, min) Rt = 2.370 min 100% ee |
| 233 | 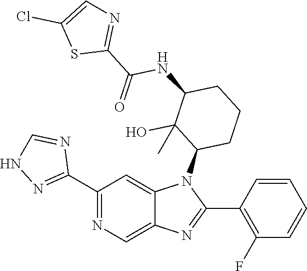 | (400 MHz, CDCl₃) δ 9.07 (br s, 1 H), 8.88 (s, 1 H), 7.74 (br s, 1 H), 7.67-7.62 (m, 2 H), 7.49-7.46 (m, 2 H), 7.42- 7.38 (m, 1 H), 7.32-7.31 (m, 1 H), 4.45 (br s, 1 H), 4.16- 4.14 (m, 1 H), 3.90-3.85 (m, 1 H), 3.08-3.06 (m, 1 H), 2.17-2.01 (m, 3 H), 1.88- 1.85 (m, 1 H), 1.64-1.57 (m, 1 H), 0.93 (br s, 3 H). | m/z 532.2 [M + H]⁺ |
| 250 | 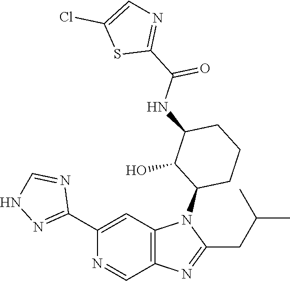 [SMILES: CC(C)Cc1nc2cnc(-c3nc[nH]n3)cc2n1[C@@H]1CCC[C@H](NC(=O)c2ncc(Cl)s2)[C@H]1O] | (400 MHz, CDCl₃) δ 8.15- 8.09 (m, 3 H), 7.68 (s, 1 H), 4.33-4.17 (m, 3 H), 2.95 (br s, 2 H), 2.38-2.33 (m, 2 H), 2.01-1.98 (m, 2 H), 1.78- 1.69 (m, 3 H), 1.27-0.90 (m, 6 H) | m/z 501.1 [M + H]⁺ (column: DAICEL CHIRALPAK IG (250 mm * 30 mm, 10 um); mobile phase: [0.1% NH₃H₂O MeOH]; B%: 60%-60%, min) Rt = 3.346 min 99.2% ee |
| 251 | 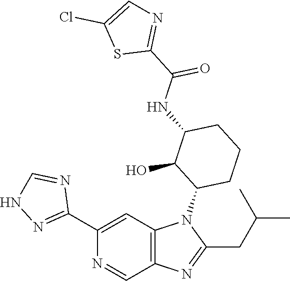 [SMILES: CC(C)Cc1nc2cnc(-c3nc[nH]n3)cc2n1[C@H]1CCC[C@@H](NC(=O)c2ncc(Cl)s2)[C@@H]1O] | (400 MHz, CDCl₃) δ 8.13- 8.09 (m, 3 H), 7.65 (s, 1 H), 4.37-4.19 (m, 3 H), 2.94 (br s, 2 H), 2.35-2.31 (m, 2 H), 2.00-1.98 (m, 2 H), 1.78- 1.69 (m, 3 H), 1.27-0.90 (m, 6 H). | m/z 501.1 [M + H]⁺ (column: DAICEL CHIRALPAK IG (250 mm * 30 mm, 10 um); mobile phase: [0.1% NH₃H₂O MeOH]; B%: 60%-60%, min) Rt = 1.444 min 99.8% ee |
| 254 | 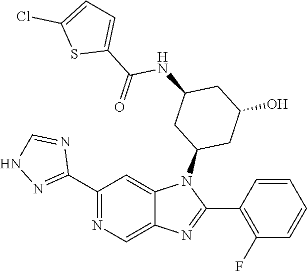 [SMILES: O=C(N[C@H]1C[C@H](O)C[C@@H](n2c(-c3ccccc3F)nc3cnc(-c4nc[nH]n4)cc32)C1)c1ccc(Cl)s1] | (400 MHz, CD₃OD) δ 9.09 (s, 1 H), 8.63 (s, 1 H), 8.35 (br s, 1 H), 7.77-7.65 (m, 2 H), 7.56-7.39 (m, 3 H), 7.00- 6.99 (m, 1 H), 4.85-4.78 (m, 1 H), 4.47-4.34 (m, 2 H), 2.61-2.56 (m, 1 H), 2.46- 2.40 (m, 1 H), 2.33-2.30 (m, 1 H), 2.14-2.10 (m, 2 H), 1.86-1.80 (m, 1 H). | m/z 538.1 [M + H]⁺ (column: DAICEL CHIRALPAK AD(250 mm * 30 mm, 10 um); mobile phase: [0.1% NH₃H₂O IPA]; B%: 35%-35%, min) Rt = 1.781 min 100% ee |
| 255 | 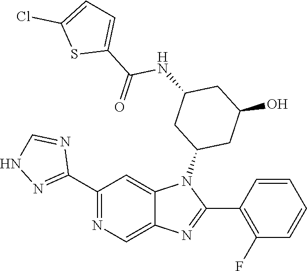 [SMILES: O=C(N[C@@H]1C[C@@H](O)C[C@H](n2c(-c3ccccc3F)nc3cnc(-c4nc[nH]n4)cc32)C1)c1ccc(Cl)s1] | (400 MHz, CD₃OD) δ 9.09 (s, 1 H), 8.63 (s, 1 H), 8.35 (br s, 1 H), 7.77-7.65 (m, 2 H), 7.56-7.39 (m, 3 H), 7.00- 6.99 (m, 1 H), 4.85-4.78 (m, 1 H), 4.47-4.34 (m, 2 H), 2.61-2.56 (m, 1 H), 2.46- 2.40 (m, 1 H), 2.33-2.30 (m, 1 H), 2.14-2.10 (m, 2 H), 1.86-1.80 (m, 1 H). | m/z 538.1 [M + H]⁺ (column: DAICEL CHIRALPAK AD(250 mm * 30 mm, 10 um); mobile phase: [0.1% NH₃H₂O IPA]; B%: 35%-35%, min) Rt = 1.781 min 100% ee |
| 256 | 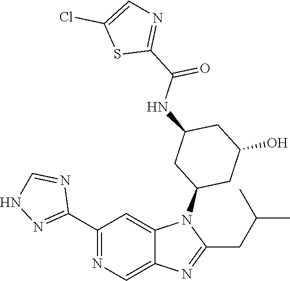 [SMILES: CC(C)Cc1nc2cnc(-c3nc[nH]n3)cc2n1[C@@H]1C[C@@H](O)C[C@H](NC(=O)c2ncc(Cl)s2)C1] | (400 MHz, CD₃OD) δ 8.94 (s, 1 H), 8.51 (s, 1 H), 8.33 (br s, 1 H), 7.81 (s, 1 H), 5.08-5.02 (m, 1 H), 4.71- 4.63 (m, 1 H), 4.45 (br s, 1 H), 3.02-2.90 (m, 2 H), 2.63- 2.48 (m, 2 H), 2.29-2.25 (m, 2 H), 2.19-2.16 (m, 1 H), 2.06-2.03 (m, 1 H), 1.99- 1.91 (m, 1 H), 1.09-1.06 (m, 6 H). | m/z 501.1 [M + H]⁺ (column: DAICEL CHIRALPAK IG (250 mm * 30 mm, 10 um); mobile phase: [0.1% NH₃H₂O IPA]; B%: 35%-35%, min) Rt = 4.032 min 96.7% ee |
| 257 | 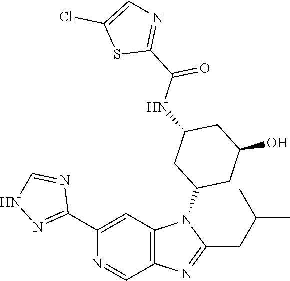 | (400 MHz, CD₃OD) δ 8.94 (s, 1 H), 8.51 (s, 1 H), 8.33 (br s, 1 H), 7.81 (s, 1 H), 5.08-5.02 (m, 1 H), 4.71- 4.63 (m, 1 H), 4.45 (br s, 1 H), 3.02-2.90 (m, 2 H), 2.63- 2.48 (m, 2 H), 2.29-2.25 (m, 2 H), 2.19-2.16 (m, 1 H), 2.06-2.03 (m, 1 H), 1.99- 1.91 (m, 1 H), 1.09-1.06 (m, 6 H). | m/z 501.1 [M + H]⁺ (column: DAICEL CHIRALPAK IG (250 mm * 30 mm, 10 um); mobile phase: [0.1% NH₃H₂O IPA]; B%: 35%-35%, min) Rt = 3.716 min 98.8% ee |
| 262 | 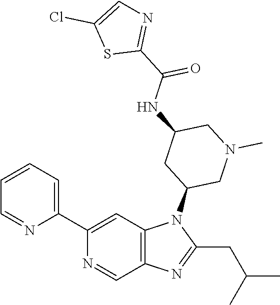 [SMILES: CC(C)Cc1nc2cnc(-c3ccccn3)cc2n1[C@H]1C[C@@H](NC(=O)c2ncc(Cl)s2)CN(C)C1] | (400 MHz, CD₃OD) δ 8.94 (s, 1 H), 8.72-8.71 (m, 1 H), 8.66 (s, 1 H), 8.46-8.44 (m, 1 H), 8.02-7.98 (m, 1 H), 7.83 (s, 1 H), 7.50-7.47 (m, 1 H), 4.83-4.77 (m, 2 H), 4.45- 4.39 (m, 1 H), 3.21-3.19 (m, 1 H), 3.05-2.96 (m, 4 H), 2.68-2.65 (m, 1 H), 2.50 (s, 3 H), 2.41-2.28 (m, 3 H), 1.11- 1.08 (m, 6 H). | m/z 510.2 [M + H]⁺ (column: DAICEL CHIRALPAK IG (250 mm * 30 mm, 10 um); mobile phase: [0.1% NH₃H₂O IPA]; B%: 50%-50%, min) Rt = 2.813 min 99.1% ee |
| 263 | 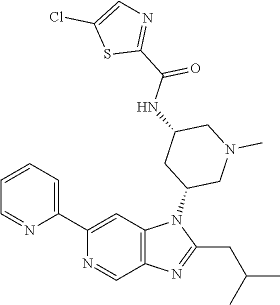 [SMILES: CC(C)Cc1nc2cnc(-c3ccccn3)cc2n1[C@@H]1C[C@H](NC(=O)c2ncc(Cl)s2)CN(C)C1] | (400 MHz, CD₃OD) δ 8.95 (s, 1 H), 8.72-8.71 (m, 1 H), 8.67 (s, 1 H), 8.46-8.44 (m, 1 H), 8.02-7.98 (m, 1 H), 7.83 (s, 1 H), 7.50-7.47 (m, 1 H), 4.83-4.77 (m, 2 H), 4.46- 4.40 (m, 1 H), 3.21-3.19 (m, 1 H), 3.06-2.96 (m, 4 H), 2.68-2.65 (m, 1 H), 2.50 (s, 3 H), 2.41-2.28 (m, 3 H), 1.12- 1.09 (m, 6 H). | m/z 510.2 [M + H]⁺ (column: DAICEL CHIRALPAK IG (250 mm * 30 mm, 10 um); mobile phase: [0.1% NH₃H₂O IPA]; B%: 50%-50%, min) Rt = 2.166 min 99.8% ee |
| 264 | 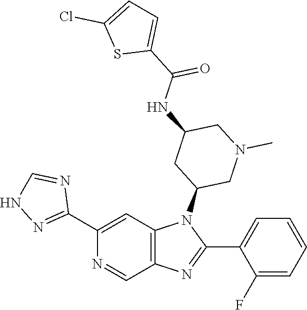 [SMILES: CN1C[C@H](NC(=O)c2ccc(Cl)s2)C[C@H](n2c(-c3ccccc3F)nc3cnc(-c4nc[nH]n4)cc32)C1] | (400 MHz, CD₃OD) δ 9.12 (s, 1 H), 8.66 (s, 1 H), 7.80- 7.74 (m, 2 H), 7.57-7.47 (m, 3 H), 7.03-7.02 (m, 1 H), 4.55-4.49 (m, 1 H), 4.21- 4.15 (m, 1 H), 3.14-3.12 (m, 2 H), 3.03-2.97 (m, 1 H), 2.51-2.44 (m, 1 H), 2.36 (s, 3 H), 2.36-2.27 (m, 2 H). | m/z 537.2 [M + H]⁺ (column: DAICEL CHIRALPAK IG (250 mm * 30 mm, 10 um); mobile phase: [0.1% NH₃H₂O IPA]; B%: 40%-40%, min) Rt = 2.214 min 97.5% ee |
| 265 | 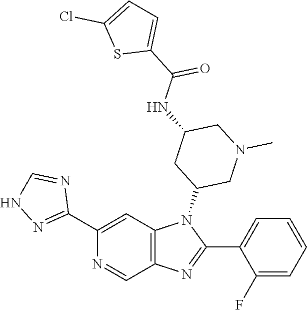 | (400 MHz, CD₃OD) δ 9.11 (s, 1 H), 8.65 (s, 1 H), 7.76- 7.72 (m, 2 H), 7.55-7.46 (m, 3 H), 7.02-7.00 (m, 1 H), 4.43-4.37 (m, 1 H), 4.09- 4.03 (m, 1 H), 3.13-3.10 (m, 2 H), 2.90-2.88 (m, 1 H), 2.49-2.45 (m, 1 H), 2.32 (s, 3 H), 2.35-2.24 (m, 2 H). | m/z 537.2 [M + H]⁺ (column: DAICEL CHIRALPAK IG (250 mm * 30 mm, 10 um); mobile phase: [0.1% NH₃H₂O IPA]; B%: 40%-40%, min) Rt = 1.885 min 97.7% ee |
| 311 | 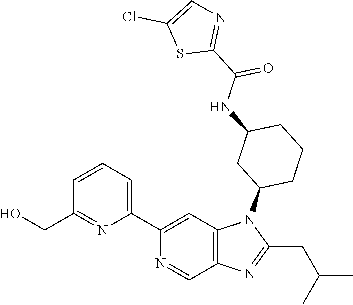 [SMILES: CC(C)Cc1nc2cnc(-c3cccc(CO)n3)cc2n1[C@@H]1CCC[C@H](NC(=O)c2ncc(Cl)s2)C1] | (400 MHz, DMSO-d₆) δ 9.09 (d, 1H), 8.95 (s, 1H), 8.63 (s, 1H), 8.29 (d, 1H), 8.05 (s, 1H), 7.94 (t, 1H), 7.51 (d, 1H), 5.48 (s, 1H), 4.75 (s, 2H), 4.72-4.61 (m, 1H), 4.26-4.13 (m, 1H), 2.90 (d, 2H), 2.57-2.51 (m, 1H), 2.29-2.05 (m, 2H), 2.05-1.87 (m, 4H), 1.75-1.53 (m, 2H), 1.02 (d, 6H). | m/z 525.2 [M + H]⁺ |
| 312 | 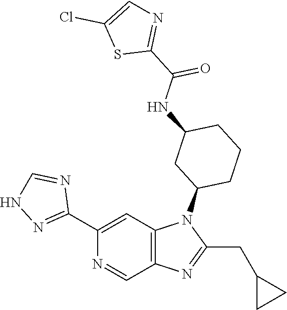 [SMILES: O=C(N[C@H]1CCC[C@@H](n2c(CC3CC3)nc3cnc(-c4nc[nH]n4)cc32)C1)c1ncc(Cl)s1] | (400 MHz, DMSO-d₆) δ 14.56 (s, 1H), 9.13 (d, 1H), 8.96 (s, 1H), 8.36 (s, 1H), 8.14-8.01 (m, 2H), 4.72-4.59 (m, 1H), 4.24-4.09 (m, 1H), 2.98 (d, 2H), 2.60-2.55 (m, 1H), 2.13-2.00 (m, 2H), 1.96-1.83 (m, 3H), 1.71-1.57 (m, 2H), 1.26-1.11 (m, 1H), 0.61-0.50 (m, 2H), 0.37-0.28 (m, 2H). | m/z 483.1 [M + H]⁺ |
| 313 | 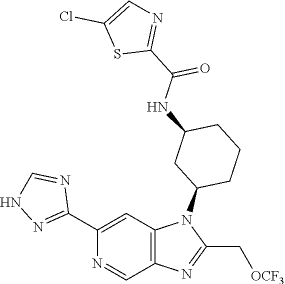 [SMILES: O=C(N[C@H]1CCC[C@@H](n2c(COC(F)(F)F)nc3cnc(-c4nc[nH]n4)cc32)C1)c1ncc(Cl)s1] | (400 MHz, DMSO-d₆) δ 14.67 (s, 1H), 9.16-9.06 (m, 2H), 8.43 (s, 1H), 8.17-7.99 (m, 2H), 5.78-5.63 (m, 2H), 4.72-4.61 (m, 1H), 4.24-4.09 (m, 1H), 2.68-2.56 (m, 1H), 2.13-1.87 (m, 5H), 1.69-1.51 (m, 2H). | m/z 527.2 [M + H]⁺ |
| 314 | 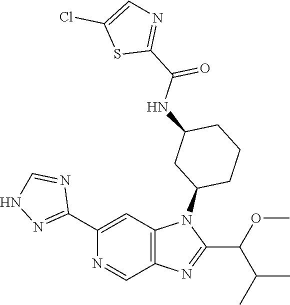 [SMILES: COC(c1nc2cnc(-c3nc[nH]n3)cc2n1[C@@H]1CCC[C@H](NC(=O)c2ncc(Cl)s2)C1)C(C)C] | (400 MHz, DMSO-d₆) δ 14.57 (s, 1H), 9.20-9.11 (m, 1H), 9.05 (s, 1H), 8.40 (s, 1H), 8.10 (s, 1H), 8.05 (s, 1H), 5.01-4.84 (m, 1H), 4.44 (t, 1H), 4.17-4.05 (m, 1H), 3.21 (s, 3H), 2.70-2.55 (m, 1H), 2.28-2.07 (m, 2H), 2.02-1.84 (m, 4H), 1.71-1.52 (m, 2H), 1.18-1.08 (m, 3H), 0.78-0.67 (m, 3H). | m/z 515.1 [M + H]⁺ |
| 316 | 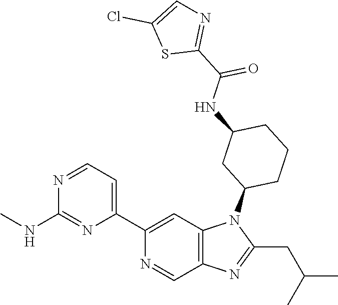 [SMILES: CNc1nccc(-c2cc3c(cn2)nc(CC(C)C)n3[C@@H]2CCC[C@H](NC(=O)c3ncc(Cl)s3)C2)n1] | (400 MHz, DMSO-d₆) δ 9.18-9.05 (m, 1H), 8.94 (s, 1H), 8.70 (s, 1H), 8.40 (s, 1H), 8.04 (s, 1H), 7.56-7.49 (m, 1H), 7.10 (s, 1H), 4.74- 4.58 (m, 1H), 4.25-4.09 (m, 1H), 2.98 (s, 3H), 2.91 (d, 2H), 2.62-2.54 (m, 1H), 2.28-2.13 (m, 2H), 2.04-1.89 (m, 4H), 1.73-1.45 (m, 2H), 1.01 (d, 6H). | m/z 525.2 [M + H]⁺ |
| 317 | 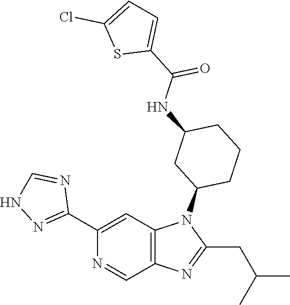 [SMILES: CC(C)Cc1nc2cnc(-c3nc[nH]n3)cc2n1[C@@H]1CCC[C@H](NC(=O)c2ccc(Cl)s2)C1] | (400 MHz, DMSO-d₆) δ 14.60 (s, 1H), 8.97 (s, 1H), 8.63 (d, 1H), 8.33 (s, 1H), 8.18-8.00 (m, 1H), 7.64 (d, 1H), 7.16 (d, 1H), 4.73-4.61 (m, 1H), 4.22-4.10 (m, 1H), 2.92 (d, 2H), 2.33-2.07 (m, 4H), 1.99-1.87 (m, 3H), 1.74-1.60 (m, 1H), 1.53-1.40 (m, 1H), 1.04-1.00 (m, 6H). | m/z 484.2 [M + H]⁺ |
| 325 | 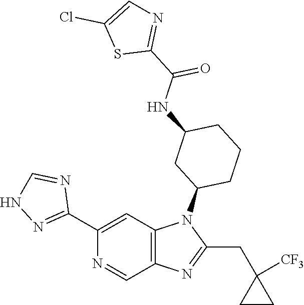 [SMILES: O=C(N[C@H]1CCC[C@@H](n2c(CC3(C(F)(F)F)CC3)nc3cnc(-c4nc[nH]n4)cc32)C1)c1ncc(Cl)s1] | (400 MHz, CD₃OD) δ 8.98 (s, 1 H), 8.56 (s, 1 H), 8.18 (br s, 1 H), 7.81 (s, 1 H), 4.69-4.63 (m, 1 H), 4.19- 4.16 (m, 1 H), 3.58-3.42 (m, 2 H), 2.57-2.32 (m, 2 H), 2.28-2.25 (m, 1 H), 2.17- 1.96 (m, 3 H), 1.80-1.63 (m, 2 H), 1.20-1.17 (m, 2 H), 0.99 (br s, 2 H). | m/z 551.1 [M + H]⁺ |
| 326 | 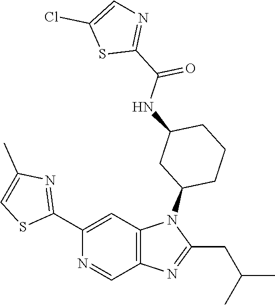 [SMILES: Cc1csc(-c2cc3c(cn2)nc(CC(C)C)n3[C@@H]2CCC[C@H](NC(=O)c3ncc(Cl)s3)C2)n1] | (400 MHz, CDCl₃) δ 8.98 (s, 1 H), 8.30 (br s, 1 H), 7.65 (s, 1 H), 7.24 (br s, 1 H), 6.98 (s, 1 H), 4.42-4.35 (m, 1 H), 4.23-4.19 (m, 1 H), 2.83-2.81 (m, 2 H), 2.58 (s, 3 H), 2.43-2.32 (m, 3 H), 2.24- 2.12 (m, 3 H), 1.98-1.95 (m, 1 H), 1.65-1.63 (m, 2 H), 1.09-1.07 (m, 6 H). | m/z 515.2 [M + H]⁺ |
| 337 | 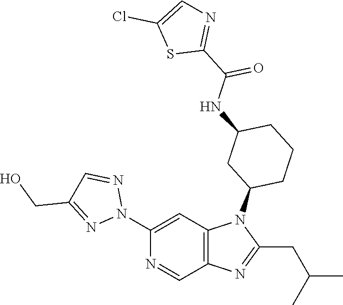 [SMILES: CC(C)Cc1nc2cnc(-n3ncc(CO)n3)cc2n1[C@@H]1CCC[C@H](NC(=O)c2ncc(Cl)s2)C1] | (400 MHz, CDCl₃) δ 8.95 (s, 1 H), 8.11 (s, 1 H), 7.91 (s, 1 H), 7.65 (s, 1 H), 7.22- 7.20 (m, 1 H), 4.96 (s, 2 H), 4.42-4.39 (m, 1 H), 4.23- 4.20 (m, 1 H), 2.85-2.83 (m, 2 H), 2.33-1.99 (m, 7 H), 1.58-1.53 (m, 2 H), 1.11- 1.09 (m, 6 H). | m/z 515.2 [M + H]⁺ |
| 338 | 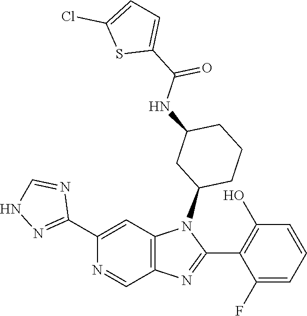 [SMILES: O=C(N[C@H]1CCC[C@@H](n2c(-c3c(O)cccc3F)nc3cnc(-c4nc[nH]n4)cc32)C1)c1ccc(Cl)s1] | (400 MHz, DMSO-d₆) δ 14.66 (s, 1H), 10.86 (s, 1H), 9.10 (s, 1H), 8.64 (d, 1H), 8.46-8.37 (m, 1H), 8.18 (s, 1H), 7.62 (d, 1H), 7.53-7.42 (m, 1H), 7.16 (d, 1H), 6.94- 6.83 (m, 2H), 4.21-4.11 (m, 1H), 3.91-3.82 (m, 1H), 2.36-2.27 (m, 1H), 2.20-2.07 (m, 2H), 1.94-1.80 (m, 3H), 1.50-1.36 (m, 2H). | m/z 538.1 [M + H]⁺ |
| 339 | 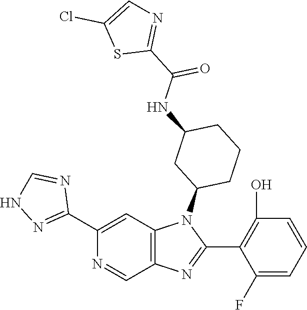 [SMILES: O=C(N[C@H]1CCC[C@@H](n2c(-c3c(O)cccc3F)nc3cnc(-c4nc[nH]n4)cc32)C1)c1ncc(Cl)s1] | (400 MHz, CD₃OD) δ 9.06 (s, 1H), 8.64 (s, 1H), 8.36- 8.09 (m, 1H), 7.80 (s, 1H), 7.52-7.44 (m, 1H), 6.92-6.81 (m, 2H), 4.30-4.18 (m, 1H), 4.01-3.92 (m, 1H), 2.57-2.22 (m, 3H), 2.11-1.96 (m, 3H), 1.72-1.37 (m, 2H). | m/z 539.1 [M + H]⁺ |
| 340 | 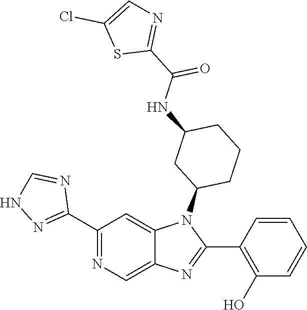 [SMILES: O=C(N[C@H]1CCC[C@@H](n2c(-c3ccccc3O)nc3cnc(-c4nc[nH]n4)cc32)C1)c1ncc(Cl)s1] | (400 MHz, DMSO-d₆) δ 14.63 (s, 1H), 10.43 (s, 1H), 9.15 (d, 1H), 9.07 (s, 1H), 8.50-8.36 (m, 1H), 8.25-8.08 (m, 1H), 8.05 (s, 1H), 7.44 (d, 2H), 7.11-6.95 (m, 2H), 4.24-4.11 (m, 1H), 3.95-3.81 (m, 1H), 2.64-2.53 (m, 1H), 2.15-1.80 (m, 5H), 1.68-1.57 (m, 1H), 1.43-1.30 (m, 1H). | m/z 521.1 [M + H]⁺ |
| 341 | 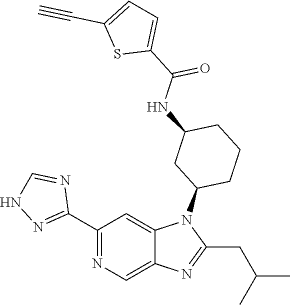 [SMILES: C#Cc1ccc(C(=O)N[C@H]2CCC[C@@H](n3c(CC(C)C)nc4cnc(-c5nc[nH]n5)cc43)C2)s1] | (400 MHz, DMSO-d₆) δ 14.62 (s, 1H), 8.97 (s, 1H), 8.69 (d, 1H), 8.33 (s, 1H), 8.07 (s, 1H), 7.68 (d, 1H), 7.35 (d, 1H), 4.72 (s, 1H), 4.71-4.62 (m, 1H), 4.23-4.12 (m, 1H), 2.92 (d, 2H), 2.33- 1.97 (m, 7H), 1.72-1.61 (m, 1H), 1.49-1.43 (m, 1H), 1.01 (d, 6H). | m/z 474.2 [M + H]⁺ |
| 342 | 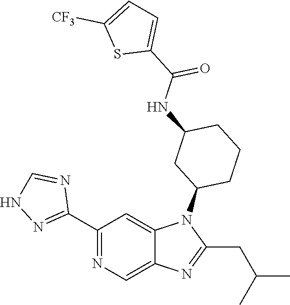 [SMILES: CC(C)Cc1nc2cnc(-c3nc[nH]n3)cc2n1[C@@H]1CCC[C@H](NC(=O)c2ccc(C(F)(F)F)s2)C1] | (400 MHz, DMSO-d₆) δ 14.61 (s, 1H), 8.98 (s, 1H), 8.88 (d, 1H), 8.34 (s, 1H), 8.07 (s, 1H), 7.84-7.80 (m, 1H), 7.76-7.72 (m, 1H), 4.73-4.61 (m, 1H), 4.28-4.14 (m, 1H), 2.92 (d, 2H), 2.37- 2.09 (m, 4H), 2.01-1.88 (m, 3H), 1.75-1.61 (m, 1H), 1.56-1.40 (m, 1H), 1.04-0.97 (m, 6H). | m/z 518.2 [M + H]⁺ |
| 343 | 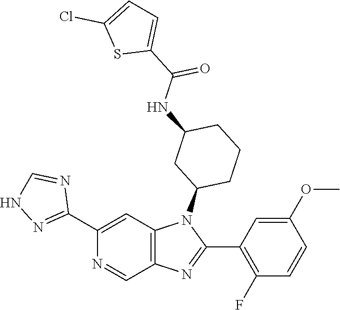 [SMILES: COc1ccc(F)c(-c2nc3cnc(-c4nc[nH]n4)cc3n2[C@@H]2CCC[C@H](NC(=O)c3ccc(Cl)s3)C2)c1] | (400 MHz, DMSO-d₆) δ 14.75 (s, 1H), 9.19 (s, 1H), 8.68 (d, 1H), 8.51 (s, 1H), 8.30-8.12 (m, 1H), 7.66 (d, 1H), 7.50 (t, 1H), 7.33-7.26 (m, 2H), 7.20 (d, 1H), 4.37- 4.27 (m, 1H), 4.01-3.90 (m, 1H), 3.88 (s, 3H), 2.42-2.31 (m, 1H), 2.26-2.15 (m, 2H), 2.05-1.91 (m, 3H), 1.55-1.45 (m, 2H). | m/z 552.2 [M + H]⁺ |
| 344 | 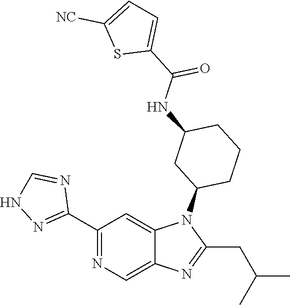 [SMILES: CC(C)Cc1nc2cnc(-c3nc[nH]n3)cc2n1[C@@H]1CCC[C@H](NC(=O)c2ccc(C#N)s2)C1] | (400 MHz, DMSO-d₆) δ 14.56 (s, 1H), 8.99-8.91 (m, 2H), 8.33 (s, 1H), 8.16-8.03 (m, 1H), 7.96 (d, 1H), 7.84 (d, 1H), 4.74-4.64 (m, 1H), 4.26-4.13 (m, 1H), 2.92 (d, 2H), 2.37-2.07 (m, 4H), 2.01-1.86 (m, 3H), 1.73-1.62 (m, 1H), 1.54-1.43 (m, 1H), 1.03-0.98 (m, 6H). | m/z 475.4 [M + H]⁺ |
| 345 | 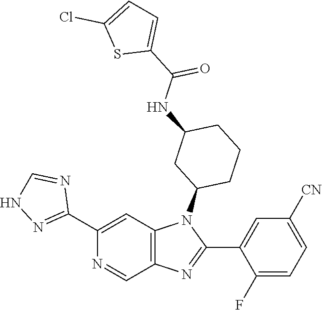 [SMILES: N#Cc1ccc(F)c(-c2nc3cnc(-c4nc[nH]n4)cc3n2[C@@H]2CCC[C@H](NC(=O)c3ccc(Cl)s3)C2)c1] | (400 MHz, DMSO-d₆) δ 14.73 (s, 1H), 9.17 (s, 1H), 8.61 (d, 1H), 8.48 (s, 1H), 8.34-8.23 (m, 2H), 8.19-8.07 (m, 1H), 7.78 (t, 1H), 7.61 (d, 1H), 7.15 (d, 1H), 4.38- 4.26 (m, 1H), 4.04-3.90 (m, 1H), 2.37-2.27 (m, 1H), 2.20-2.07 (m, 2H), 2.02-1.96 (m, 1H), 1.92-1.84 (m, 2H), 1.57-1.40 (m, 2H). | m/z 547.2 [M + H]⁺ |
| 347 | 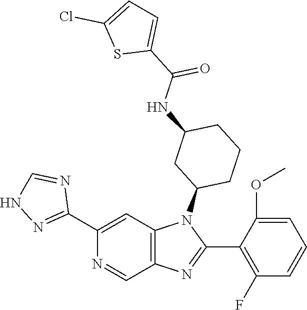 [SMILES: COc1cccc(F)c1-c1nc2cnc(-c3nc[nH]n3)cc2n1[C@@H]1CCC[C@H](NC(=O)c2ccc(Cl)s2)C1] | (400 MHz, DMSO-d₆) δ 14.71 (s, 1H), 9.12 (s, 1H), 8.67-8.57 (m, 1H), 8.50-8.39 (m, 1H), 8.10 (s, 1H), 7.75- 7.56 (m, 2H), 7.19-7.02 (m, 3H), 4.21-4.04 (m, 1H), 3.94-3.86 (m, 1H), 3.83 (s, 3H), 2.26-1.78 (m, 6H), 1.54-1.38 (m, 2H). | m/z 552.1 [M + H]⁺ |
| 348 | 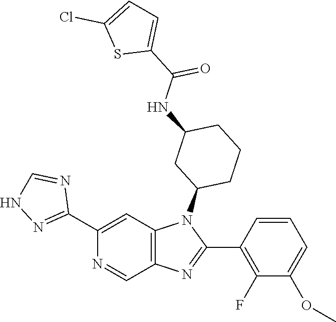 [SMILES: COc1cccc(-c2nc3cnc(-c4nc[nH]n4)cc3n2[C@@H]2CCC[C@H](NC(=O)c3ccc(Cl)s3)C2)c1F] | (400 MHz, DMSO-d₆) δ 14.61 (s, 1H), 9.14 (s, 1H), 8.63 (d, 1H), 8.46 (s, 1H), 8.25 (s, 1H), 7.62 (d, 1H), 7.48 (t, 1H), 7.39 (t, 1H), 7.22 (t, 1H), 7.15 (d, 1H), 4.34-4.19 (m, 1H), 3.96 (s, 3H), 3.92-3.81 (m, 1H), 2.37-2.26 (m, 1H), 2.18-2.07 (m, 2H), 1.98-1.85 (m, 3H), 1.48-1.39 (m, 2H). | m/z 552.1 [M + H]⁺ |
| 349 | 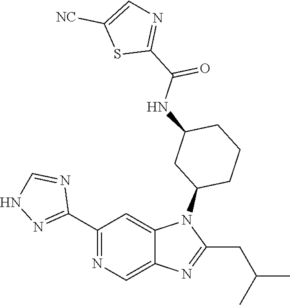 [SMILES: CC(C)Cc1nc2cnc(-c3nc[nH]n3)cc2n1[C@@H]1CCC[C@H](NC(=O)c2ncc(C#N)s2)C1] | (400 MHz, DMSO-d₆) δ 14.60 (s, 1H), 9.39 (d, 1H), 8.96 (s, 1H), 8.80 (s, 1H), 8.37 (s, 1H), 8.19-7.97 (m, 1H), 4.76-4.61 (m, 1H), 4.33-4.17 (m, 1H), 2.91 (d, 2H), 2.28-2.17 (m, 1H), 2.13-1.81 (m, 6H), 1.65 (s, 2H), 1.10-0.96 (m, 6H). | m/z 476.2 [M + H]⁺ |
| 351 | 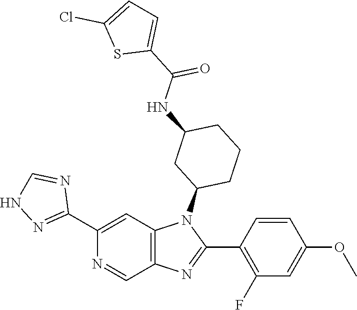 [SMILES: COc1ccc(-c2nc3cnc(-c4nc[nH]n4)cc3n2[C@@H]2CCC[C@H](NC(=O)c3ccc(Cl)s3)C2)c(F)c1] | (400 MHz, DMSO-d₆) δ 14.61 (s, 1H), 9.11 (s, 1H), 8.63 (d, 1H), 8.44 (s, 1H), 8.18 (s, 1H), 7.67-7.59 (m, 2H), 7.18-7.10 (m, 2H), 7.06-6.99 (m, 1H), 4.33-4.19 (m, 1H), 3.96-3.89 (m, 4H), 2.35-2.27 (m, 1H), 2.19-2.09 (m, 2H), 2.00-1.88 (m, 3H), 1.51-1.41 (m, 2H). | m/z 552.1 [M + H]⁺ |
| 352 | 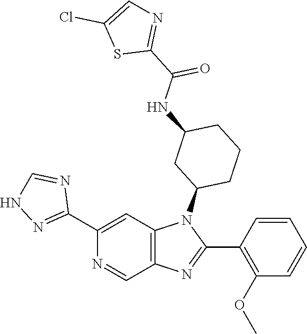 [SMILES: COc1ccccc1-c1nc2cnc(-c3nc[nH]n3)cc2n1[C@@H]1CCC[C@H](NC(=O)c2ncc(Cl)s2)C1] | (400 MHz, DMSO-d₆) δ 14.60 (s, 1H), 9.14-9.04 (m, 2H), 8.44 (s, 1H), 8.13 (s, 1H), 8.04 (s, 1H), 7.68-7.61 (m, 1H), 7.53 (dd, 1H), 7.30 (d, 1H), 7.17 (t, 1H), 4.15- 4.01 (m, 1H), 3.98-3.82 (m, 4H), 2.19-1.58 (m, 7H), 1.54-1.31 (m, 1H). | m/z 535.1 [M + H]⁺ |
| 353 | 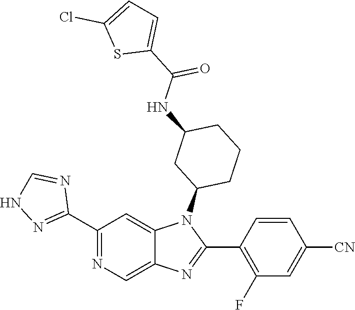 [SMILES: N#Cc1ccc(-c2nc3cnc(-c4nc[nH]n4)cc3n2[C@@H]2CCC[C@H](NC(=O)c3ccc(Cl)s3)C2)c(F)c1] | (400 MHz, DMSO-d₆) δ 15.00-14.48 (m, 1H), 9.17 (s, 1H), 8.60 (d, 1H), 8.48 (s, 1H), 8.22 (d, 2H), 8.01-7.90 (m, 2H), 7.61 (d, 1H), 7.15 (d, 1H), 4.39-4.27 (m, 1H), 4.04-3.87 (m, 1H), 2.35-2.24 (m, 1H), 2.20-2.05 (m, 2H), 2.01-1.91 (m, 1H), 1.93-1.82 (m, 2H), 1.59-1.39 (m, 2H). | m/z 547.2 [M + H]⁺ |
| 354 | 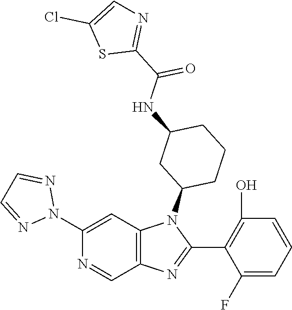 [SMILES: O=C(N[C@H]1CCC[C@@H](n2c(-c3c(O)cccc3F)nc3cnc(-n4nccn4)cc32)C1)c1ncc(Cl)s1] | (400 MHz, DMSO-d₆) δ 10.85-10.38 (m, 1H), 9.08- 9.00 (m, 1H), 8.97 (s, 1H), 8.30 (s, 1H), 8.20 (s, 2H), 8.05 (s, 1H), 7.52-7.40 (m, 1H), 6.95-6.79 (m, 2H), 4.26-4.12 (m, 1H), 4.03-3.85 (m, 1H), 2.59-2.53 (m, 1H), 2.13-1.76 (m, 5H), 1.65-1.36 (m, 2H). | m/z 539.1 [M + H]⁺ |
| 355 | 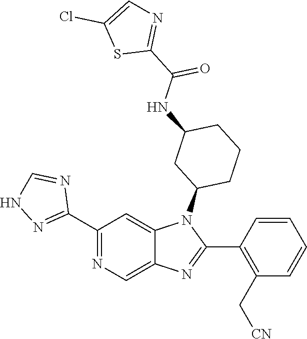 [SMILES: N#CCc1ccccc1-c1nc2cnc(-c3nc[nH]n3)cc2n1[C@@H]1CCC[C@H](NC(=O)c2ncc(Cl)s2)C1] | (400 MHz, CD₃OD) δ 15.39 (s, 1H), 10.00-9.91 (m, 2H), 9.32 (s, 1H), 8.97 (s, 1H), 8.86 (s, 1H), 8.54-8.48 (m, 2H), 8.47-8.40 (m, 2H), 5.12-4.97 (m, 1H), 4.92 (s, 2H), 4.76-4.62 (m, 1H), 3.51-3.36 (m, 1H), 3.01-2.80 (m, 3H), 2.75-2.56 (m, 2H), 2.48-2.35 (m, 1H), 2.28-2.15 (m, 1H). | m/z 544.1 [M + H]⁺ |
| 356 | 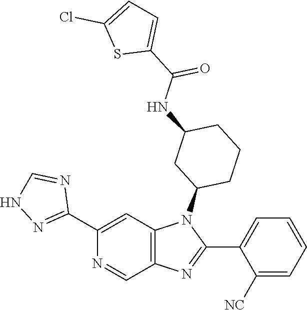 [SMILES: N#Cc1ccccc1-c1nc2cnc(-c3nc[nH]n3)cc2n1[C@@H]1CCC[C@H](NC(=O)c2ccc(Cl)s2)C1] | (400 MHz, DMSO-d₆) δ 14.66 (s, 1H), 9.19 (s, 1H), 8.63 (d, 1H), 8.50 (s, 1H), 8.16 (d, 2H), 8.02-7.93 (m, 1H), 7.93-7.86 (m, 2H), 7.62 (d, 1H), 7.16 (d, 1H), 4.36- 4.23 (m, 1H), 3.96-3.80 (m, 1H), 2.37-2.30 (m, 1H), 2.24-2.06 (m, 3H), 1.92-1.83 (m, 2H), 1.48-1.39 (m, 2H). | m/z 529.2 [M + H]⁺ |
| 358 | 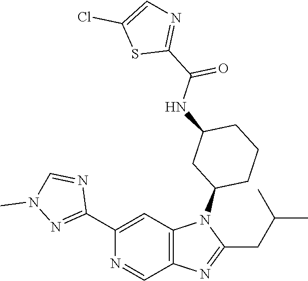 [SMILES: CC(C)Cc1nc2cnc(-c3ncn(C)n3)cc2n1[C@@H]1CCC[C@H](NC(=O)c2ncc(Cl)s2)C1] | (400 MHz, CDCl₃) δ 9.11 (s, 1 H), 8.27 (s, 1 H), 8.16 (s, 1 H), 7.64 (s, 1 H), 7.24- 7.22 (m, 1 H), 4.40-4.38 (m, 1 H), 4.21-4.20 (m, 1 H), 2.84-2.82 (m, 2 H), 2.39- 2.01 (m, 7 H), 1.62-1.56 (m, 2 H), 1.10-1.08 (m, 6 H). | m/z 499.2 [M + H]⁺ |
| 364 | 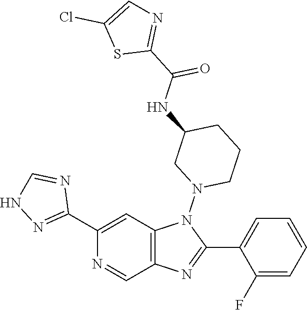 [SMILES: O=C(N[C@H]1CCCN(n2c(-c3ccccc3F)nc3cnc(-c4nc[nH]n4)cc32)C1)c1ncc(Cl)s1] | (400 MHz, DMSO-d₆) δ 14.67 (s, 1H), 9.15 (s, 1H), 9.02 (d, 1H), 8.50 (s, 1H), 8.15 (s, 1H), 8.05 (s, 1H), 7.74-7.67 (m, 2H), 7.49-7.35 (m, 2H), 3.95-3.84 (m, 1H), 3.81-3.70 (m, 1H), 3.51-3.42 (m, 1H), 3.28-3.18 (m, 2H), 1.90-1.80 (m, 2H), 1.76-1.64 (m, 1H), 1.50-1.38 (m, 1H). | m/z 547.1 [M + H]⁺ |
| 365 | 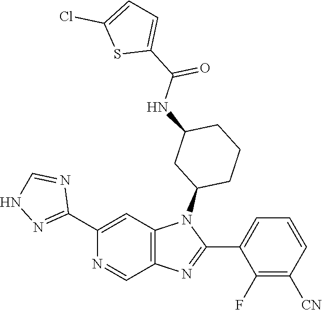 [SMILES: N#Cc1cccc(-c2nc3cnc(-c4nc[nH]n4)cc3n2[C@@H]2CCC[C@H](NC(=O)c3ccc(Cl)s3)C2)c1F] | (400 MHz, DMSO-d₆) δ 14.66 (s, 1H), 9.17 (s, 1H), 8.62 (d, 1H), 8.48 (s, 1H), 8.26 (t, 1H), 8.09 (t, 1H), 7.70-7.52 (m, 3H), 7.15 (d, 1H), 4.47-4.36 (m, 1H), 4.09-3.95 (m, 1H), 2.38-2.28 (m, 1H), 2.19-2.10 (m, 2H), 2.02-1.95 (m, 1H), 1.93-1.82 (m, 2H), 1.62-1.41 (m, 2H). | m/z 547.1 [M + H]⁺ |
| 366 | 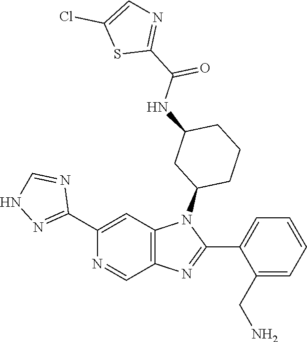 [SMILES: NCc1ccccc1-c1nc2cnc(-c3nc[nH]n3)cc2n1[C@@H]1CCC[C@H](NC(=O)c2ncc(Cl)s2)C1] | (400 MHz, CDCl₃) δ 9.17 (br s, 1 H), 8.61 (br s, 1 H), 8.41 (br s, 1 H), 8.14 (br s, 1 H), 7.77 (br s, 1 H), 7.58 (br s, 1 H), 7.55-7.48 (m, 3 H), 7.33 (br s, 1 H), 4.34-4.31 (m, 1 H), 4.03-4.94 (m, 3 H), 2.41- 2.26 (m, 4 H), 2.06-1.95 (m, 4 H), 1.60-1.52 (m, 1 H), 1.40 (br s, 1 H). | m/z 534.2 [M + H]⁺ |
| 367 | 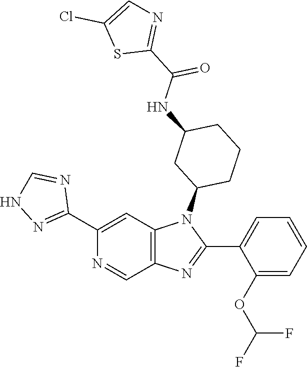 [SMILES: O=C(N[C@H]1CCC[C@@H](n2c(-c3ccccc3OC(F)F)nc3cnc(-c4nc[nH]n4)cc32)C1)c1ncc(Cl)s1] | (400 MHz, DMSO-d₆) δ 14.70 (s, 1H), 9.18-9.07 (m, 2H), 8.48 (s, 1H), 8.10 (s, 1H), 8.04 (s, 1H), 7.79-7.66 (m, 2H), 7.49 (d, 2H), 7.47- 7.15 (m, 1H), 4.19-4.08 (m, 1H), 3.95-3.83 (m, 1H), 2.64-2.55 (m, 1H), 2.18-1.77 (m, 5H), 1.65-1.54 (m, 1H), 1.47-1.34 (m, 1H). | m/z 571.1 [M + H]⁺ |
| 368 | 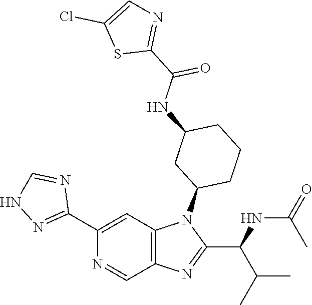 [SMILES: CC(=O)N[C@H](c1nc2cnc(-c3nc[nH]n3)cc2n1[C@@H]1CCC[C@H](NC(=O)c2ncc(Cl)s2)C1)C(C)C] | (400 MHz, CD₃OD) δ 8.98 (s, 1H), 8.55 (s, 1H), 8.37- 8.08 (m, 1H), 7.80 (s, 1H), 5.12 (d, 1H), 4.97-4.89 (m, 1H), 4.30-4.17 (m, 1H), 2.56-2.44 (m, 2H), 2.38-2.20 (m, 2H), 2.15-1.96 (m, 6H), 1.78-1.68 (m, 2H), 1.16 (d, 3H), 0.93 (d, 3H). | m/z 542.4 [M + H]⁺; (column: ChiralCel OD (250 mm * 4.6 mm, 5 um); mobile phase: [0.05% DEA MeOH]; B%: 8 min @ 40%); Rt = 2.75 min >99% ee |
| 369 | 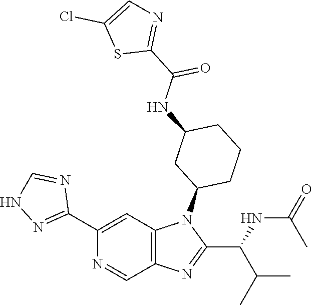 [SMILES: CC(=O)N[C@@H](c1nc2cnc(-c3nc[nH]n3)cc2n1[C@@H]1CCC[C@H](NC(=O)c2ncc(Cl)s2)C1)C(C)C] | (400 MHz, DMSO-d₆) δ 14.61 (s, 1H), 9.11 (d, 1H), 9.02 (s, 1H), 8.51 (d, 1H), 8.36 (s, 1H), 8.10-8.02 (m, 2H), 5.07 (t, 1H), 4.82-4.67 (m, 1H), 4.18-4.00 (m, 1H), 2.45-2.38 (m, 1H), 2.16-1.85 (m, 9H), 1.73-1.49 (m, 2H), 1.07 (d, 3H), 0.89 (d, 3H). | m/z 542.2 [M + H]⁺; (column: ChiralPAK G-IG (100 mm * 4.6 mm, 5 um); mobile phase: [0.05% DEA MeOH]; B%: 8 min @ 40%); Rt = 2.268 min 64% ee |
| 370 | 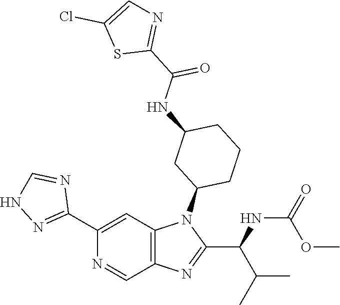 [SMILES: COC(=O)N[C@H](c1nc2cnc(-c3nc[nH]n3)cc2n1[C@@H]1CCC[C@H](NC(=O)c2ncc(Cl)s2)C1)C(C)C] | (400 MHz, DMSO-d₆) δ 14.61 (s, 1H), 9.17 (d, 1H), 9.01 (s, 1H), 8.37 (s, 1H), 8.12-8.02 (m, 2H), 7.93 (d, 1H), 4.89-4.74 (m, 2H), 4.23-4.07 (m, 1H), 3.54 (s, 3H), 2.71-2.59 (m, 1H), 2.44-2.31 (m, 1H), 2.08-1.80 (m, 5H), 1.70-1.48 (m, 2H), 1.08 (d, 3H), 0.86 (d, 3H). | m/z 558.4 [M + H]⁺; (column: ChiralPAK G-IG (100 mm * 4.6 mm, 5 um); mobile phase: [0.05% DEA MeOH]; B%: 8 min @ 40%); Rt = 1.48 min 80% ee |
| 371 | 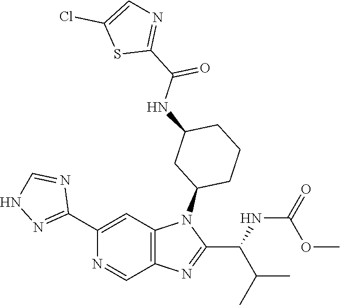 [SMILES: COC(=O)N[C@@H](c1nc2cnc(-c3nc[nH]n3)cc2n1[C@@H]1CCC[C@H](NC(=O)c2ncc(Cl)s2)C1)C(C)C] | (400 MHz, DMSO-d₆) δ 14.58 (s, 1H), 9.16 (dd, 1H), 9.01 (s, 1H), 8.40 (s, 1H), 8.18-8.03 (m, 2H), 7.96-7.86 (m, 1H), 4.86-4.75 (m, 2H), 4.22-4.06 (m, 1H), 3.53 (s, 3H), 2.60-2.53 (m, 1H), 2.43-2.32 (m, 1H), 2.21-2.08 (m, 1H), 2.01-1.84 (m, 4H), 1.67-1.55 (m, 2H), 1.08 (d, 3H), 0.88 (d, 3H). | m/z 558.2 [M + H]⁺; (column: ChiralPak C-IG (100 mm * 4.6 mm, 5 um); mobile phase: [0.05% DEA MeOH]; B%: 8 min @ 40%); Rt = 2.943 min 81% ee |
| 372 | 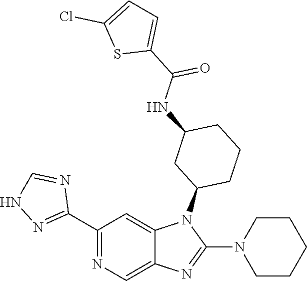 [SMILES: O=C(N[C@H]1CCC[C@@H](n2c(N3CCCCC3)nc3cnc(-c4nc[nH]n4)cc32)C1)c1ccc(Cl)s1] | (400 MHz, DMSO-d₆) δ 8.79 (s, 1H), 8.66 (d, 1H), 8.17 (s, 1H), 8.05 (s, 1H), 7.62 (d, 1H), 7.16 (d, 1H), 4.33-4.20 (m, 1H), 4.08-3.93 (m, 1H), 3.23-3.15 (m, 4H), 2.27 (q, 1H), 2.14-2.00 (m, 2H), 2.01-1.85 (m, 3H), 1.77-1.68 (m, 4H), 1.66-1.40 (m, 4H). | m/z 511.2 [M + H]⁺ |
| 375 | 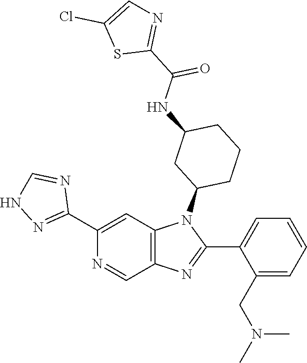 [SMILES: CN(C)Cc1ccccc1-c1nc2cnc(-c3nc[nH]n3)cc2n1[C@@H]1CCC[C@H](NC(=O)c2ncc(Cl)s2)C1] | (400 MHz, CDCl₃) δ 9.09 (s, 1 H), 8.47 (s, 1 H), 8.12 (s, 1 H), 7.63 (s, 1 H), 7.61 (br s, 1 H), 7.59-7.57 (m, 1 H), 7.50-7.48 (m, 1 H), 7.33- 7.31 (m, 1 H), 7.23-7.20 (m, 1 H), 4.11-4.04 (m, 1 H), 3.94-3.88 (m, 1 H), 2.38- 2.35 (m, 1 H), 2.29-2.24 (m, 3 H), 2.10 (br s, 6 H), 2.01- 1.99 (m, 2 H), 1.54-1.48 (m, 2 H), 1.43-1.33 (m, 2 H). | m/z 562.2 [M + H]⁺ |
| 376 | 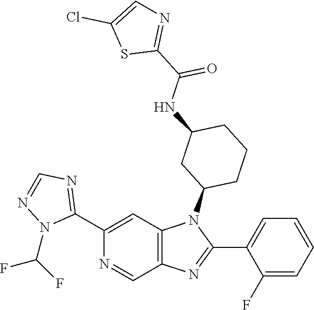 [SMILES: O=C(N[C@H]1CCC[C@@H](n2c(-c3ccccc3F)nc3cnc(-c4ncnn4C(F)F)cc32)C1)c1ncc(Cl)s1] | (400 MHz, DMSO-d₆) δ 9.20 (s, 1H), 9.09-9.05 (m, 1H), 8.84 (t, 1H), 8.63 (s, 1H), 8.45 (s, 1H), 8.05 (s, 1H), 7.78-7.71 (m, 2H), 7.58-7.44 (m, 2H), 4.39-4.19 (m, 1H), 4.05-3.80 (m, 1H), 2.67-2.54 (m, 1H), 2.11-2.02 (m, 2H), 1.98-1.77 (m, 3H), 1.64-1.52 (m, 1H), 1.50-1.38 (m, 1H). | m/z 573.4 [M + H]⁺ (column: ChiralPak IB (100 mm * 4.6 mm, 5 um); mobile phase: [0.05% DEA MeOH]; B%: 8 min @ 30%); Rt = 1.458 min >99% ee |
| 377 | 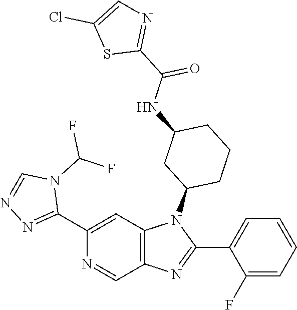 [SMILES: O=C(N[C@H]1CCC[C@@H](n2c(-c3ccccc3F)nc3cnc(-c4nncn4C(F)F)cc32)C1)c1ncc(Cl)s1] | (400 MHz, DMSO-d₆) δ 9.33 (s, 1H), 9.17 (s, 1H), 9.06 (d, 1H), 8.83-8.45 (m, 2H), 8.04 (s, 1H), 7.79-7.68 (m, 2H), 7.60-7.41 (m, 2H), 4.37-4.19 (m, 1H), 4.06-3.89 (m, 1H), 2.70-2.54 (m, 1H), 2.15-2.01 (m, 2H), 2.00-1.77 (m, 3H), 1.70-1.53 (m, 1H), 1.53-1.37 (m, 1H). | m/z 573.4 [M + H]⁺; (column: ChiralPak AD (250 mm * 4.6 mm, 5 um); mobile phase: [0.05% DEA MeOH]; B%: 8 min @ 40%); Rt = 2.093 min >99% ee |
| 378 | 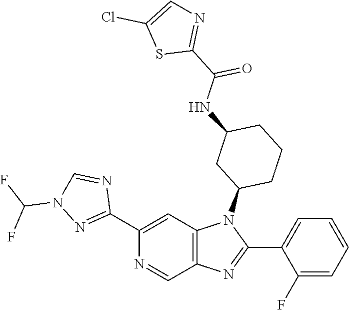 [SMILES: O=C(N[C@H]1CCC[C@@H](n2c(-c3ccccc3F)nc3cnc(-c4ncn(C(F)F)n4)cc32)C1)c1ncc(Cl)s1] | (400 MHz, DMSO-d₆) δ 9.26 (s, 1H), 9.15-9.07 (m, 2H), 8.50 (s, 1H), 8.30-7.92 (m, 2H), 7.80-7.67 (m, 2H), 7.58-7.38 (m, 2H), 4.34-4.14 (m, 1H), 4.02-3.79 (m, 1H), 2.13-1.79 (m, 6H), 1.62-1.46 (m, 2H). | m/z 573.5 [M + H]⁺; (column: ChiralPak C-IG (100 mm * 4.6 mm, 5 um); mobile phase: [0.05% DEA MeOH]; B%: 8 min @ 40%); Rt = 3.321 min >99% ee |
| 379 | 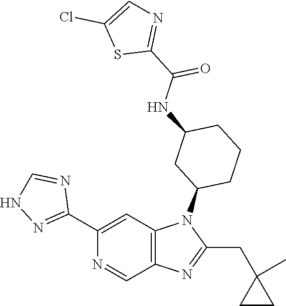 [SMILES: CC1(Cc2nc3cnc(-c4nc[nH]n4)cc3n2[C@@H]2CCC[C@H](NC(=O)c3ncc(Cl)s3)C2)CC1] | (400 MHz, DMSO-d₆) δ 14.58 (s, 1H), 9.13-9.06 (m, 1H), 8.99 (s, 1H), 8.36 (s, 1H), 8.04 (s, 2H), 4.72-4.59 (m, 1H), 4.24-4.11 (m, 1H), 3.04 (s, 2H), 2.65-2.54 (m, 1H), 2.14-1.97 (m, 2H), 1.95-1.84 (m, 3H), 1.71-1.59 (m, 2H), 1.08 (s, 3H), 0.63- 0.56 (m, 2H), 0.46-0.37 (m, 2H). | m/z 497.2 [M + H]⁺ |
| 382 | 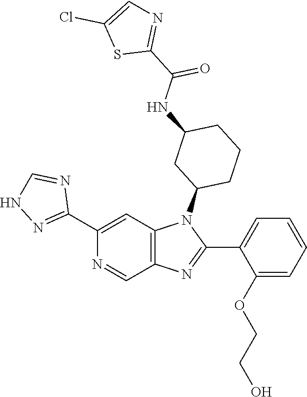 [SMILES: O=C(N[C@H]1CCC[C@@H](n2c(-c3ccccc3OCCO)nc3cnc(-c4nc[nH]n4)cc32)C1)c1ncc(Cl)s1] | (400 MHz, DMSO-d₆) δ 14.65 (s, 1H), 9.18-9.01 (m, 2H), 8.44 (s, 1H), 8.17-8.00 (m, 2H), 7.64-7.56 (m, 1H), 7.48 (dd, 1H), 7.32 (d, 1H), 7.15 (t, 1H), 4.75 (s, 1H), 4.21-4.04 (m, 3H), 3.97-3.77 (m, 1H), 3.68-3.53 (m, 2H), 2.31-1.73 (m, 5H), 1.70-1.20 (m, 3H). | m/z 565.0 [M + H]⁺ |
| 384 | 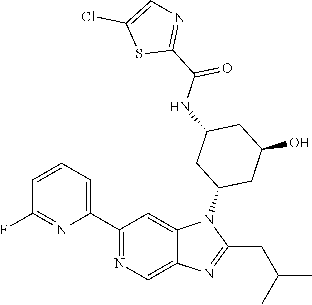 [SMILES: CC(C)Cc1nc2cnc(-c3cccc(F)n3)cc2n1[C@H]1C[C@H](O)C[C@@H](NC(=O)c2ncc(Cl)s2)C1] | (400 MHz, CDCl₃) δ 9.08 (s, 1 H), 8.48 (br s, 2 H), 7.96- 7.94 (m, 1 H), 7.66 (s, 1 H), 7.32-7.30 (m, 1 H), 6.97- 6.95 (m, 1 H), 5.03 (br s, 1 H), 4.74-4.71 (m, 1 H), 4.59 (s, 1 H), 2.91-2.89 (m, 2 H), 2.55-2.29 (m, 5 H), 2.14- 2.11 (m, 1 H), 1.90-1.86 (m, 2 H), 1.11-1.08 (m, 6 H). | m/z 529.2 [M + H]⁺; (column: DAICEL CHIRALPAK AD (250 mm * 30 mm, 10 um); mobile phase: [0.1% NH₃H₂O EtOH]; B%: 25%-25%, min). Rt = 4.001 min 94.7% ee |
| 385 | 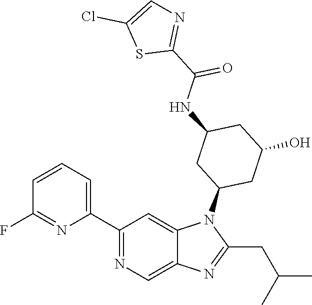 [SMILES: CC(C)Cc1nc2cnc(-c3cccc(F)n3)cc2n1[C@@H]1C[C@@H](O)C[C@H](NC(=O)c2ncc(Cl)s2)C1] | (400 MHz, CDCl₃) δ 9.09 (s, 1 H), 8.49 (br s, 2 H), 7.96- 7.94 (m, 1 H), 7.66 (s, 1 H), 7.32-7.30 (m, 1 H), 6.97- 6.95 (m, 1 H), 5.03 (br s, 1 H), 4.74-4.71 (m, 1 H), 4.59 (s, 1 H), 2.91-2.89 (m, 2 H), 2.55-2.30 (m, 5 H), 2.14- 2.11 (m, 1 H), 1.90-1.86 (m, 2 H), 1.12-1.09 (m, 6 H). | m/z 529.2 [M + H]⁺; (column: DAICEL CHIRALPAK AD (250 mm * 30 mm, 10 um); mobile phase: [0.1% NH₃H₂O EtOH]; B%: 25%-25%, min). Rt = 4.257 min 98.7% ee |
| 386 | 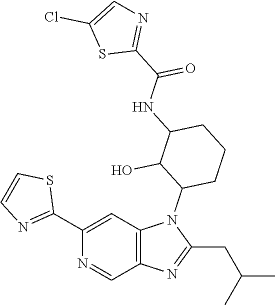 [SMILES: CC(C)Cc1nc2cnc(-c3nccs3)cc2n1C1CCCC(NC(=O)c2ncc(Cl)s2)C1O] | (400 MHz, DMSO-d₆) δ 9.01 (d, 1H), 8.89 (s, 1H), 8.37 (s, 1H), 8.04 (s, 1H), 7.97 (d, 1H), 7.80 (d, 1H), 5.09 (d, 1H), 4.43-4.32 (m, 2H), 4.08-3.96 (m, 1H), 2.96-2.78 (m, 2H), 2.39-2.19 (m, 2H), 1.91-1.63 (m, 5H), 1.05-0.98 (m, 6H). | m/z 517.2 [M + H]⁺ |
| | (relative stereochemistry at | | |
| | centers 1 and 3 of the | | |
| | cyclohexyl ring is cis with | | |
| | —OH at center 2 trans) | | |
| 389 | 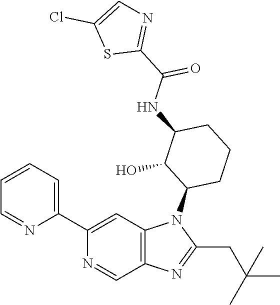 [SMILES: CC(C)(C)Cc1nc2cnc(-c3ccccn3)cc2n1[C@@H]1CCC[C@H](NC(=O)c2ncc(Cl)s2)[C@H]1O] | (400 MHz, DMSO-d₆) δ 8.99 (d, 1H), 8.95 (s, 1H), 8.73 (d, 1H), 8.65 (s, 1H), 8.45 (d, 1H), 8.03 (s, 1H), 7.96-7.89 (m, 1H), 7.45-7.37 (m, 1H), 5.00 (d, 1H), 4.51-4.40 (m, 1H), 4.36-4.26 (m, 1H), 4.14-4.00 (m, 1H), 3.16-3.06 (m, 1H), 2.83-2.73 (m, 1H), 2.41-2.32 (m, 1H), 1.92-1.60 (m, 5H), 1.10 (s, 9H). | m/z 525.2 [M + H]⁺; (column: ChiralPak IH (100 mm * 4.6 mm, 5 um); mobile phase: [0.05% DEA iPrOH]; B%: 8 min @ 30%); Rt = 1.266 min 98.3% ee |
| 390 | 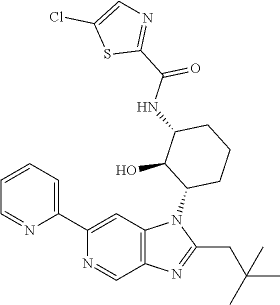 [SMILES: CC(C)(C)Cc1nc2cnc(-c3ccccn3)cc2n1[C@H]1CCC[C@@H](NC(=O)c2ncc(Cl)s2)[C@@H]1O] | (400 MHz, DMSO-d₆) δ 9.00 (d, 1H), 8.95 (s, 1H), 8.73 (d, 1H), 8.65 (s, 1H), 8.45 (d, 1H), 8.03 (s, 1H), 7.96-7.89 (m, 1H), 7.43-7.38 (m, 1H), 5.00 (d, 1H), 4.50-4.40 (m, 1H), 4.38-4.27 (m, 1H), 4.13-4.02 (m, 1H), 3.16-3.08 (m, 1H), 2.79-2.73 (m, 1H), 2.42-2.29 (m, 1H), 1.96-1.64 (m, 5H), 1.10 (s, 9H). | m/z 525.2 [M + H]⁺; (column: ChiralPak IH (100 mm * 4.6 mm, 5 um); mobile phase: [0.05% DEA iPrOH]; B%: 8 min @ 30%); Rt = 2.118 min 95% ee |
| 399 | 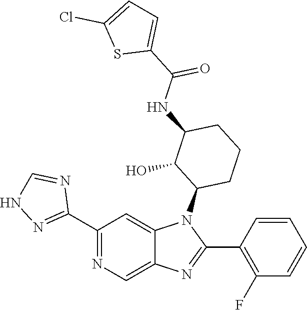 [SMILES: O=C(N[C@H]1CCC[C@@H](n2c(-c3ccccc3F)nc3cnc(-c4nc[nH]n4)cc32)[C@@H]1O)c1ccc(Cl)s1] | (400 MHz, DMSO-d₆) δ 14.61 (s, 1H), 9.13 (s, 1H), 8.54 (d, 1H), 8.44 (s, 1H), 8.22 (s, 1H), 7.74-7.67 (m, 2H), 7.58 (d, 1H), 7.52-7.43 (m, 2H), 7.14 (d, 1H), 5.23 (d, 1H), 4.22-4.13 (m, 1H), 4.06-3.97 (m, 1H), 3.74-3.63 (m, 1H), 2.44-2.29 (m, 1H), 2.19-2.09 (m, 1H), 1.90-1.78 (m, 2H), 1.69-1.41 (m, 2H). | m/z 538.1 [M + H]⁺ (column: ChiralPak IH (100 mm * 4.6 mm, 5 um); mobile phase: [0.05% DEA iPrOH]; B%: 8 min @ 30%); Rt = 1.499 min >99% ee |
| 400 | 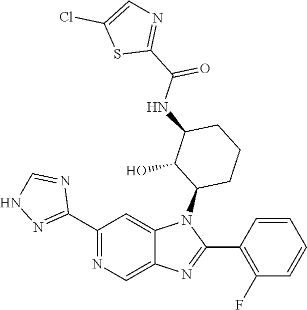 [SMILES: O=C(N[C@H]1CCC[C@@H](n2c(-c3ccccc3F)nc3cnc(-c4nc[nH]n4)cc32)[C@@H]1O)c1ncc(Cl)s1] | (400 MHz, DMSO-d₆) δ 14.63 (s, 1H), 9.12 (s, 1H), 8.95 (d, 1H), 8.47 (s, 1H), 8.15 (s, 1H), 8.02 (s, 1H), 7.70 (t, 2H), 7.52-7.42 (m, 2H), 5.17 (d, 1H), 4.45-4.34 (m, 1H), 4.05-3.93 (m, 1H), 3.75-3.63 (m, 1H), 2.35-2.25 (m, 1H), 2.17-2.08 (m, 1H), 1.89-1.75 (m, 3H), 1.51-1.35 (m, 1H). | m/z 539.1 [M + H]⁺ (column: ChiralPak IH (100 mm * 4.6 mm, 5 um); mobile phase: [0.05% DEA iPrOH]; B%: 8 min @ 50%); Rt = 2.892 min >99% ee |
| 401 | 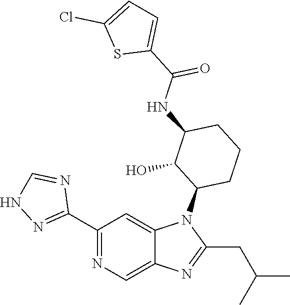 [SMILES: CC(C)Cc1nc2cnc(-c3nc[nH]n3)cc2n1[C@@H]1CCC[C@H](NC(=O)c2ccc(Cl)s2)[C@H]1O] | (400 MHz, DMSO-d₆) δ 14.60 (s, 1H), 8.97 (s, 1H), 8.58 (d, 1H), 8.29 (s, 1H), 8.07 (s, 1H), 7.62 (d, 1H), 7.19-7.14 (m, 1H), 5.14 (d, 1H), 4.44-4.34 (m, 1H), 4.20-4.11 (m, 1H), 4.08-3.94 (m, 1H), 2.99-2.81 (m, 2H), 2.39-2.18 (m, 2H), 1.98-1.81 (m, 3H), 1.73-1.55 (m, 2H), 1.05-1.00 (m, 6H). | m/z 500.2 [M + H]⁺ |
| 402 | 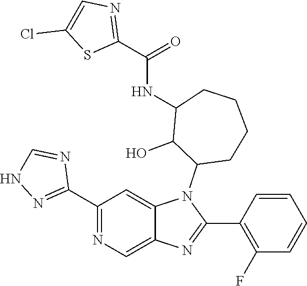 [SMILES: O=C(NC1CCCCC(n2c(-c3ccccc3F)nc3cnc(-c4nc[nH]n4)cc32)C1O)c1ncc(Cl)s1] | (400 MHz, CDCl₃) δ 8.97 (s, 1H), 8.25 (s, 1H), 7.96 (s, 1H), 7.77 (s, 1H), 7.65-7.55 (m, 2H), 7.37 (t, 1H) 7.30- 7.22 (m, 2H overlaps with chloroform solvent peak), 4.35-4.16 (m, 2H), 3.95-3.82 (m, 1H), 2.58 (s, 1H), 2.26 (d, 1H), 2.03-1.52 (m, 8H, overlaps with water peak). | m/z 553.1 [M + H]⁺ |
| 403 | 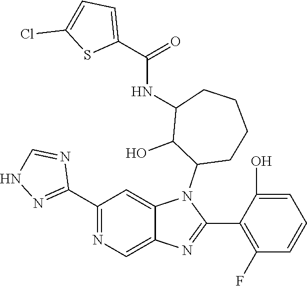 [SMILES: O=C(NC1CCCCC(n2c(-c3c(O)cccc3F)nc3cnc(-c4nc[nH]n4)cc32)C1O)c1ccc(Cl)s1] | (400 MHz, CD₃OD) δ 9.05 (s, 1H), 8.57 (s, 1H), 7.50- 7.41 (m, 2H), 6.95 (d, 1H), 6.89-6.78 (m, 2H), 4.23 (t, 1H), 4.14 (t, 1H), 3.80 (t, 1H), 2.81-2.64 (m, 1H), 2.34-2.16 (m, 1H), 2.09-2.00 (m, 1H), 1.97-1.60 (m, 5H). | m/z 568.1 [M + H]⁺ |
| 404 | 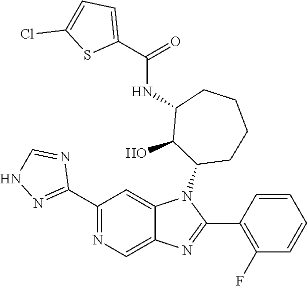 [SMILES: O=C(N[C@@H]1CCCC[C@H](n2c(-c3ccccc3F)nc3cnc(-c4nc[nH]n4)cc32)[C@H]1O)c1ccc(Cl)s1] | (400 MHz, CD₃OD) δ 9.07 (s, 1H), 8.59 (s, 1H), 8.33 (s, 1H), 7.77-7.66 (m, 2H), 7.48-7.36 (m, 3H), 6.95 (d, 1H), 4.31-4.10 (m, 2H), 3.80 (t, 1H), 2.87-2.68 (m, 1H), 2.24 (d, 1H), 2.10-1.57 (m, 6H). | m/z 552.1 [M + H]⁺ (column: Diacel ChiralPak AD-H (250 mm * 20 mm); mobile phase: [0.1% DEA 50% iPrOH]; Rt = 3.64 min 100% ee |
| 405 | 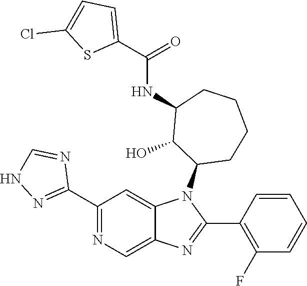 [SMILES: O=C(N[C@H]1CCCC[C@@H](n2c(-c3ccccc3F)nc3cnc(-c4nc[nH]n4)cc32)[C@@H]1O)c1ccc(Cl)s1] | (400 MHz, CD₃OD) δ 9.07 (s, 1H), 8.59 (s, 1H), 8.33 (s, 1H), 7.77-7.66 (m, 2H), 7.48-7.36 (m, 3H), 6.95 (d, 1H), 4.31-4.10 (m, 2H), 3.80 (t, 1H), 2.87-2.68 (m, 1H), 2.24 (d, 1H), 2.10-1.57 (m, 6H). | m/z 552.1 [M + H]⁺ (column: Diacel ChiralPak AD-H (250 mm * 20 mm); mobile phase: [0.1% DEA 50% iPrOH]; Rt = 6.59 min 98.4% ee |
| 406 | 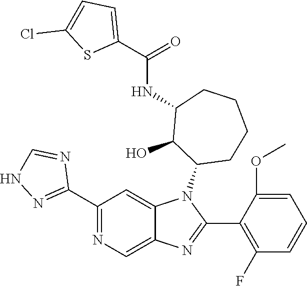 [SMILES: COc1cccc(F)c1-c1nc2cnc(-c3nc[nH]n3)cc2n1[C@H]1CCCC[C@@H](NC(=O)c2ccc(Cl)s2)[C@@H]1O] | (400 MHz, CD₃OD) δ 9.04 (s, 1H), 8.57 (s, 1H), 8.32 (s, 1H), 7.69-7.57 (m, 1H), 7.47 (dd, 1H), 7.11-7.04 (m, 1H), 7.00-6.92 (m, 2H), 4.20 (t, 1H), 3.98 (t, 1H), 3.89 (s, 3H), 3.77 (t, 1H), 2.84-2.66 (m, 1H), 2.20 (d, 1H), 2.12- 1.76 (m, 4H), 1.75-1.59 (m, 2H). | m/z 582.1 [M + H]⁺ (column: Diacel ChiralPak AD-H (250 mm * 20 mm); mobile phase: [0.1% DEA 50% iPrOH]; Rt = 1.96 min 100% ee |
| 407 | 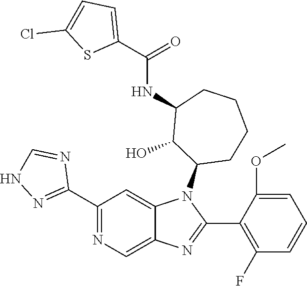 [SMILES: COc1cccc(F)c1-c1nc2cnc(-c3nc[nH]n3)cc2n1[C@@H]1CCCC[C@H](NC(=O)c2ccc(Cl)s2)[C@H]1O] | (400 MHz, CD₃OD) δ 9.04 (s, 1H), 8.57 (s, 1H), 8.32 (s, 1H), 7.69-7.57 (m, 1H), 7.47 (dd, 1H), 7.11-7.04 (m, 1H), 7.00-6.92 (m, 2H), 4.20 (t, 1H), 3.98 (t, 1H), 3.89 (s, 3H), 3.77 (t, 1H), 2.84-2.66 (m, 1H), 2.20 (d, 1H), 2.12- 1.76 (m, 4H), 1.75-1.59 (m, 2H). | m/z 582.1 [M + H]⁺ (column: Diacel ChiralPak AD-H (250 mm * 20 mm); mobile phase: [0.1% DEA 50% iPrOH]; Rt = 3.09 min 100% ee |
| 408 | 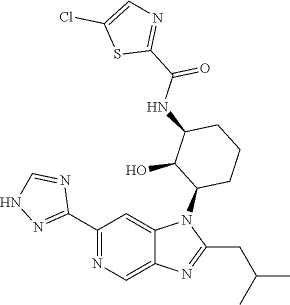 [SMILES: CC(C)Cc1nc2cnc(-c3nc[nH]n3)cc2n1[C@@H]1CCC[C@H](NC(=O)c2ncc(Cl)s2)[C@@H]1O] | (400 MHz, DMSO-d₆) δ 14.50 (s, 1H), 8.91 (s, 1H), 8.60 (s, 1H), 8.12-8.00 (m, 2H), 5.76 (d, 1H), 4.72-4.60 (m, 1H), 4.31-4.19 (m, 1H), 4.08-4.01 (m, 1H), 2.98-2.84 (m, 2H), 2.73-2.64 (m, 1H), 2.34-2.22 (m, 1H), 1.93-1.77 (m, 2H), 1.76-1.62 (m, 3H), 1.07-0.98 (m, 6H). | m/z 501.2 [M + H]⁺ (column: ChiralPak C-IG (100 mm * 4.6 mm, 5 um); mobile phase: [0.05% DEA MeOH]; B%: 8 min @ 40%); Rt = 2.248 min 100% ee |
| 409 | 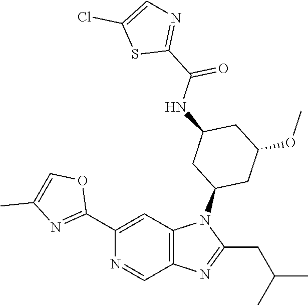 [SMILES: CO[C@H]1C[C@H](NC(=O)c2ncc(Cl)s2)C[C@H](n2c(CC(C)C)nc3cnc(-c4nc(C)co4)cc32)C1] | (400 MHz, CDCl₃) δ 9.08 (s, 1 H), 8.24 (s, 1 H), 7.65 (s, 1 H), 7.55 (s, 1 H), 7.22-7.20 (m, 1 H), 4.89-4.84 (m, 1 H), 4.56-4.52 (m, 1 H), 3.90 (br s, 1 H), 3.43 (s, 3 H), 2.85- 2.84 (m, 2 H), 2.52-2.49 (m, 1 H), 2.49-2.16 (m, 8 H), 1.69-1.64 (m, 1 H), 1.10- 1.07 (m, 6 H). | m/z 529.2 [M + H]⁺; (column: DAICEL CHIRALCEL OD (250 mm * 30 mm, 10 um); mobile phase: [0.1% NH₃H₂O EtOH]; B%: 20%-20%, min). Rt = 3.350 min 100% ee |
| 410 | 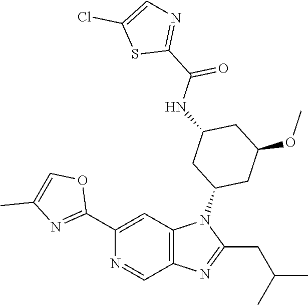 [SMILES: CO[C@@H]1C[C@@H](NC(=O)c2ncc(Cl)s2)C[C@@H](n2c(CC(C)C)nc3cnc(-c4nc(C)co4)cc32)C1] | (400 MHz, CDCl₃) δ 9.06 (s, 1 H), 8.23 (s, 1 H), 7.65 (s, 1 H), 7.55 (s, 1 H), 7.22-7.20 (m, 1 H), 4.89-4.84 (m, 1 H), 4.56-4.52 (m, 1 H), 3.90 (br s, 1 H), 3.42 (s, 3 H), 2.86- 2.84 (m, 2 H), 2.52-2.49 (m, 1 H), 2.49-2.16 (m, 8 H), 1.66-1.61 (m, 1 H), 1.09- 1.06 (m, 6 H). | m/z 529.2 [M + H]⁺; (column: DAICEL CHIRALCEL OD (250 mm * 30 mm, 10 um); mobile phase: [0.1% NH₃H₂O EtOH]; B%: 20%-20%, min). Rt = 3.624 min 97.9% ee |
| 411 | 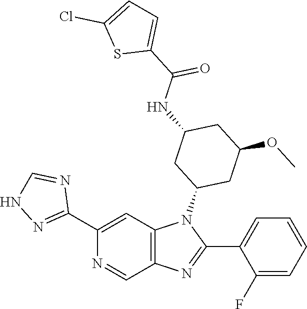 [SMILES: CO[C@@H]1C[C@@H](NC(=O)c2ccc(Cl)s2)C[C@@H](n2c(-c3ccccc3F)nc3cnc(-c4nc[nH]n4)cc32)C1] | (400 MHz, CDCl₃) δ 9.21 (s, 1 H), 8.46 (s, 1 H), 8.21 (s, 1 H), 7.69-7.60 (m, 2 H), 7.51- 7.49 (m, 1 H), 7.44-7.34 (m, 1 H), 7.35-7.29 (m, 1 H), 6.88-6.87 (m, 1 H), 6.78 (br s, 1 H), 4.69-4.63 (m, 1 H), 4.38 (br s, 1 H), 3.72 (br s, 1 H), 3.23 (s, 3 H), 2.33-2.22 (m, 6 H). | m/z 552.2 [M + H]⁺; (column: DAICEL CHIRALPAK IG (250 mm * 30 mm, 10 um); mobile phase: [0.1% NH₃H₂O IPA]; B%: 45%-45%, min). Rt = 4.201 min 93.2% ee |
| 412 | 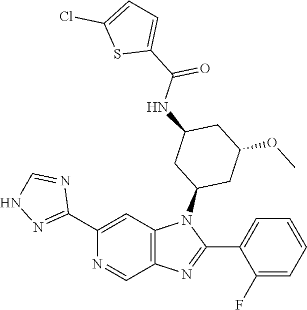 [SMILES: CO[C@H]1C[C@H](NC(=O)c2ccc(Cl)s2)C[C@H](n2c(-c3ccccc3F)nc3cnc(-c4nc[nH]n4)cc32)C1] | (400 MHz, CDCl₃) δ 9.21 (s, 1 H), 8.46 (s, 1 H), 8.21 (s, 1 H), 7.69-7.60 (m, 2 H), 7.51- 7.49 (m, 1 H), 7.44-7.34 (m, 1 H), 7.35-7.29 (m, 1 H), 6.88-6.87 (m, 1 H), 6.78 (br s, 1 H), 4.69-4.63 (m, 1 H), 4.38 (br s, 1 H), 3.70 (br s, 1 H), 3.22 (s, 3 H), 2.31-2.18 (m, 6 H). | m/z 552.2 [M + H]⁺; (column: DAICEL CHIRALPAK IG (250 mm * 30 mm, 10 um); mobile phase: [0.1% NH₃H₂O IPA]; B%: 45%-45%, min). Rt = 5.180 min 100% ee |
| 413 | 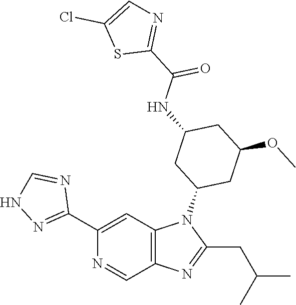 [SMILES: CO[C@@H]1C[C@@H](NC(=O)c2ncc(Cl)s2)C[C@@H](n2c(CC(C)C)nc3cnc(-c4nc[nH]n4)cc32)C1] | (400 MHz, CDCl₃) δ 9.01 (s, 1 H), 8.37 (s, 1 H), 8.09 (s, 1 H), 7.58 (s, 1 H), 7.31-7.30 (m, 1 H), 4.86-4.74 (m, 1 H), 4.57-4.40 (m, 1 H), 3.83 (br s, 1 H), 3.36 (s, 3 H), 2.85- 2.76 (m, 2 H), 2.48-2.28 (m, 4 H), 2.22-2.21 (m, 1 H), 2.12-2.09 (m, 1 H), 1.69- 1.63 (m, 1 H), 1.06-1.03 (m, 6 H). | m/z 515.2 [M + H]⁺; (column: DAICEL CHIRALPAK IG (250 mm * 30 mm, 10 um); mobile phase: [0.1% NH₃H₂O IPA]; B%: 45%-45%, min). Rt = 4.088 min 100% ee |
| 414 | 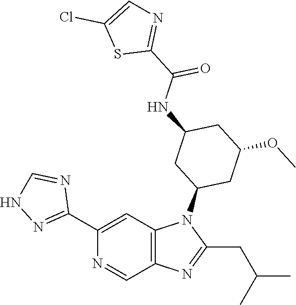 [SMILES: CO[C@H]1C[C@H](NC(=O)c2ncc(Cl)s2)C[C@H](n2c(CC(C)C)nc3cnc(-c4nc[nH]n4)cc32)C1] | (400 MHz, CDCl₃) δ 9.01 (s, 1 H), 8.37 (s, 1 H), 8.09 (s, 1 H), 7.58 (s, 1 H), 7.31-7.30 (m, 1 H), 4.86-4.74 (m, 1 H), 4.57-4.40 (m, 1 H), 3.83 (br s, 1 H), 3.36 (s, 3 H), 2.85- 2.76 (m, 2 H), 2.48-2.28 (m, 4 H), 2.22-2.21 (m, 1 H), 2.12-2.09 (m, 1 H), 1.69- 1.63 (m, 1 H), 1.06-1.03 (m, 6 H). | m/z 515.2 [M + H]⁺; (column: DAICEL CHIRALPAK IG (250 mm * 30 mm, 10 um); mobile phase: [0.1% NH₃H₂O IPA]; B%: 45%-45%, min). Rt = 4.907 min 97.6% ee |
| 419 | 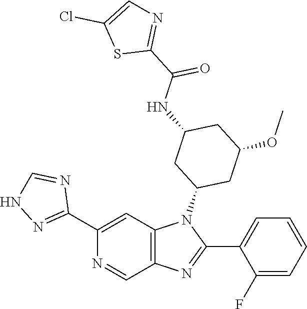 [SMILES: CO[C@H]1C[C@@H](NC(=O)c2ncc(Cl)s2)C[C@@H](n2c(-c3ccccc3F)nc3cnc(-c4nc[nH]n4)cc32)C1] | (400 MHz, CDCl₃) δ 9.22 (s, 1 H), 8.59 (s, 1 H), 8.26 (s, 1 H), 7.70-7.66 (m, 3 H), 7.45- 7.41 (m, 1 H), 7.38-7.32 (m, 2 H), 4.30-4.27 (m, 1 H), 4.11-4.09 (m, 1 H), 3.42- 3.36 (m, 4 H), 2.55-2.28 (m, 5 H), 1.66-1.60 (m, 1 H). | m/z 553.1 [M + H]⁺; (column: DAICEL CHIRALPAK AD (250 mm * 30 mm, 10 um); mobile phase: [0.1% NH₃H₂O IPA]; B%: 40%-40%, min). Rt = 1.062 min 100% ee |
| 420 | 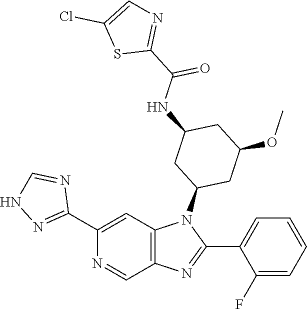 [SMILES: CO[C@@H]1C[C@H](NC(=O)c2ncc(Cl)s2)C[C@H](n2c(-c3ccccc3F)nc3cnc(-c4nc[nH]n4)cc32)C1] | (400 MHz, CDCl₃) δ 9.21 (s, 1 H), 8.57 (s, 1 H), 8.23 (s, 1 H), 7.70-7.64 (m, 3 H), 7.45- 7.41 (m, 1 H), 7.36-7.31 (m, 2 H), 4.30-4.27 (m, 1 H), 4.11-4.09 (m, 1 H), 3.42- 3.36 (m, 4 H), 2.55-2.31 (m, 5 H), 1.65-1.59 (m, 1 H). | m/z 553.2 [M + H]⁺; (column: DAICEL CHIRALPAK AD (250 mm * 30 mm, 10 um); mobile phase: [0.1% NH₃H₂O IPA]; B%: 40%-40%, min). Rt = 1.282 min 95.9% ee |
| 431 | 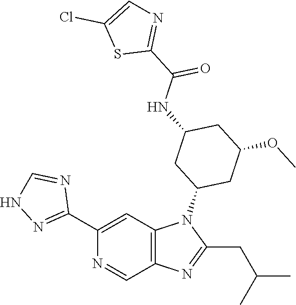 [SMILES: CO[C@H]1C[C@@H](NC(=O)c2ncc(Cl)s2)C[C@@H](n2c(CC(C)C)nc3cnc(-c4nc[nH]n4)cc32)C1] | (400 MHz, CD₃OD) δ 8.95 (s, 1 H), 8.54 (s, 1 H), 8.31 (br s, 1 H), 7.82 (s, 1 H), 4.70 (br s, 1 H), 4.26 (br s, 1 H), 3.65 (br s, 1 H), 3.43 (s, 3 H), 2.99-2.97 (m, 2 H), 2.47-1.99 (m, 6 H), 1.74- 1.68 (m, 1 H), 1.09-1.07 (m, 6 H). | m/z 515.2 [M + H]⁺; (column: DAICEL CHIRALPAK IG (250 mm * 30 mm, 10 um); mobile phase: [0.1% NH₃H₂O IPA]; B%: 40%-40%, min). Rt = 1.821 min 98.6% ee |
| 432 | 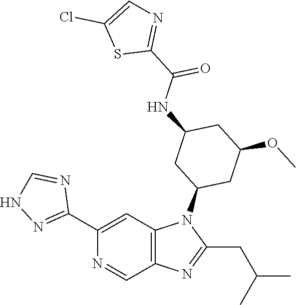 [SMILES: CO[C@@H]1C[C@H](NC(=O)c2ncc(Cl)s2)C[C@H](n2c(CC(C)C)nc3cnc(-c4nc[nH]n4)cc32)C1] | (400 MHz, CD₃OD) δ 8.95 (s, 1 H), 8.54 (s, 1 H), 8.32 (br s, 1 H), 7.82 (s, 1 H), 4.70 (br s, 1 H), 4.29-4.23 (m, 1 H), 3.67-3.65 (m, 1 H), 3.43 (s, 3 H), 2.99-2.97 (m, 2 H), 2.47-1.99 (m, 6 H), 1.74- 1.68 (m, 1 H), 1.09-1.07 (m, 6 H). | m/z 515.2 [M + H]⁺; (column: DAICEL CHIRALPAK IG (250 mm * 30 mm, 10 um); mobile phase: [0.1% NH₃H₂O IPA]; B%: 40%-40%, min). Rt = 2.131 min 98.2% ee |
| 433 | 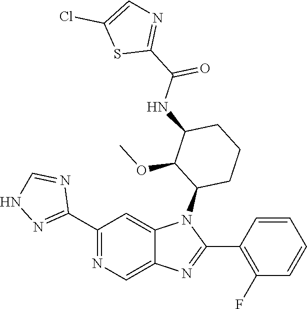 [SMILES: CO[C@H]1[C@@H](NC(=O)c2ncc(Cl)s2)CCC[C@H]1n1c(-c2ccccc2F)nc2cnc(-c3nc[nH]n3)cc21] | (400 MHz, DMSO-d₆) δ 14.36 (s, 1H), 9.09 (d, 1H), 8.97 (s, 1H), 8.53 (s, 1H), 8.03 (s, 1H), 7.80-7.65 (m, 2H), 7.61 (s, 1H), 7.56-7.47 (m, 2H), 4.86-4.75 (m, 1H), 4.72-4.61 (m, 1H), 4.16-4.07 (m, 1H), 3.14 (s, 3H), 2.44- 2.38 (m, 1H), 2.28-2.15 (m, 1H), 1.68-1.49 (m, 3H), 1.19-1.06 (m, 1H). | m/z 553.1 [M + H]⁺; (column: ChiralPak C-IG (100 mm * 4.6 mm, 5 um); mobile phase: [0.05% DEA iPrOH]; B%: 8 min @ 20%); Rt = 5.555 min >99% ee |
| 434 | 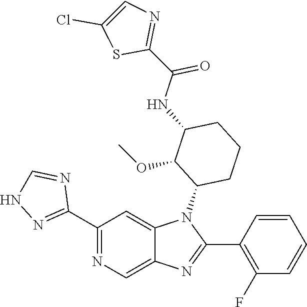 [SMILES: CO[C@@H]1[C@H](NC(=O)c2ncc(Cl)s2)CCC[C@@H]1n1c(-c2ccccc2F)nc2cnc(-c3nc[nH]n3)cc21] | (400 MHz, DMSO-d₆) δ 14.45 (s, 1H), 9.15 (d, 1H), 9.03 (s, 1H), 8.59 (s, 1H), 8.03 (s, 1H), 7.83-7.70 (m, 2H), 7.67 (s, 1H), 7.62-7.52 (m, 2H), 4.94-4.81 (m, 1H), 4.78-4.67 (m, 1H), 4.23-4.12 (m, 1H), 3.19 (s, 3H), 2.48- 2.43 (m, 1H), 2.33-2.19 (m, 1H), 1.72-1.50 (m, 3H), 1.24-1.12 (m, 1H). | m/z 553.1 [M + H]⁺; (column: ChiralPak C-IG (100 mm * 4.6 mm, 5 um); mobile phase: [0.05% DEA iPrOH]; B%: 8 min @ 20%); Rt = 10.916 min 98% ee |
| 435 | 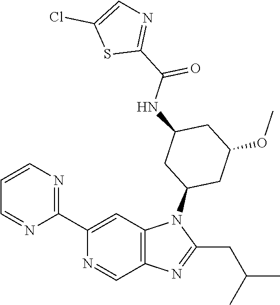 [SMILES: CO[C@H]1C[C@H](NC(=O)c2ncc(Cl)s2)C[C@H](n2c(CC(C)C)nc3cnc(-c4ncccn4)cc32)C1] | (400 MHz, DMSO-d₆) δ 9.10 (d, 1H), 8.99 (s, 3H), 8.70- 8.55 (m, 1H), 8.06 (s, 1H), 7.59-7.44 (m, 1H), 4.79-4.66 (m, 1H), 4.44-4.31 (m, 1H), 3.90-3.85 (m, 1H), 3.34 (s, 3H), 2.92-2.81 (m, 2H), 2.69-2.62 (m, 1H), 2.27-2.09 (m, 5H), 1.85-1.75 (m, 1H), 1.05 (d, 3H), 1.01 (d, 3H). | m/z 526.2 [M + H]⁺; (column: ChiralCel OD (150 mm * 4.6 mm, 3 um); mobile phase: [0.05% DEA EtOH]; B%: 40%); Rt = 1.265 min 99% ee |
| 436 | 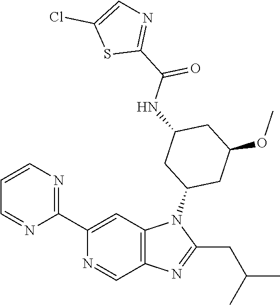 [SMILES: CO[C@@H]1C[C@@H](NC(=O)c2ncc(Cl)s2)C[C@@H](n2c(CC(C)C)nc3cnc(-c4ncccn4)cc32)C1] | (400 MHz, DMSO-d₆) δ 9.10 (d, 1H), 8.99 (s, 3H), 8.69- 8.60 (m, 1H), 8.06 (s, 1H), 7.62-7.48 (m, 1H), 4.79-4.66 (m, 1H), 4.44-4.30 (m, 1H), 3.91-3.83 (m, 1H), 2.92-2.82 (m, 2H), 2.29-2.09 (m, 6H), 1.84-1.74 (m, 1H), 1.05 (d, 3H), 1.01 (d, 3H). | m/z 526.2 [M + H]⁺; (column: ChiralCel OD (150 mm * 4.6 mm, 3 um); mobile phase: [0.05% DEA EtOH]; B%: 40%); Rt = 1.726 min 99% ee |
| 437 | 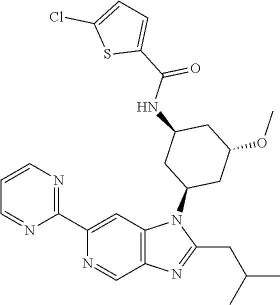 [SMILES: CO[C@H]1C[C@H](NC(=O)c2ccc(Cl)s2)C[C@H](n2c(CC(C)C)nc3cnc(-c4ncccn4)cc32)C1] | (400 MHz, DMSO-d₆) δ 9.18-8.84 (m, 2H), 8.60 (d, 2H), 7.62 (d, 1H), 7.57-7.40 (m, 1H), 7.16 (d, 1H), 4.78- 4.67 (m, 1H), 4.38-4.26 (m, 1H), 3.91-3.85 (m, 1H), 2.97-2.80 (m, 2H), 2.40-2.15 (m, 6H), 1.69-1.57 (m, 1H), 1.05 (d, 3H), 1.01 (d, 3H). | m/z 525.2 [M + H]⁺; (column: ChiralCel OD (150 mm * 4.6 mm, 3 um); mobile phase: [0.05% DEA EtOH]; B%: 5-40%); Rt = 1.199 min 99% ee |
| 438 | 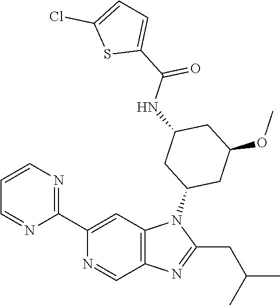 [SMILES: CO[C@@H]1C[C@@H](NC(=O)c2ccc(Cl)s2)C[C@@H](n2c(CC(C)C)nc3cnc(-c4ncccn4)cc32)C1] | (400 MHz, DMSO-d₆) δ 8.98 (s, 2H), 8.60 (d, 2H), 7.62 (d, 1H), 7.53 (s, 1H), 7.16 (d, 1H), 4.77-4.67 (m, 1H), 4.40-4.26 (m, 1H), 3.93-3.82 (m, 1H), 2.95-2.81 (m, 2H), 2.39-2.13 (m, 6H), 1.69-1.57 (m, 1H), 1.05 (d, 3H), 1.01 (d, 3H). | m/z 525.2 [M + H]⁺; (column: ChiralCel OD (150 mm * 4.6 mm, 3 um); mobile phase: [0.05% DEA EtOH]; B%: 5-40%); Rt = 1.668 min 99% ee |
| 441 | 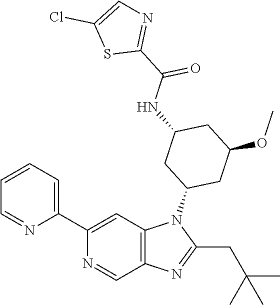 [SMILES: CO[C@@H]1C[C@@H](NC(=O)c2ncc(Cl)s2)C[C@@H](n2c(CC(C)(C)C)nc3cnc(-c4ccccn4)cc32)C1] | (400 MHz, CD₃OD) δ 8.94 (s, 1H), 8.68-8.62 (m, 2H), 8.42 (d, 1H), 8.02-7.93 (m, 1H), 7.79 (s, 1H), 7.48-7.42 (m, 1H), 5.05-4.95 (m, 1H), 4.54-4.42 (m, 1H), 3.98-3.91 (m, 1H), 3.45 (s, 3H), 3.05- 2.86 (m, 2H), 2.71-2.55 (m, 2H), 2.42-2.33 (m, 1H), 2.29-2.18 (m, 2H), 1.96-1.78 (m, 1H), 1.14 (s, 9H). | m/z 539.4 [M + H]⁺; (column: ChiralPak AD (150 mm * 4.6 mm, 3 um); mobile phase: [0.05% DEA iPrOH]; B%: 5-40%); Rt = 3.195 min >99% ee |
| 442 | 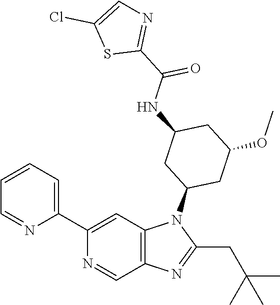 [SMILES: CO[C@H]1C[C@H](NC(=O)c2ncc(Cl)s2)C[C@H](n2c(CC(C)(C)C)nc3cnc(-c4ccccn4)cc32)C1] | (400 MHz, CD₃OD) δ 8.94 (s, 1H), 8.69-8.63 (m, 2H), 8.43 (d, 1H), 8.02-7.94 (m, 1H), 7.79 (s, 1H), 7.48-7.42 (m, 1H), 5.07-4.95 (m, 1H), 4.55-4.43 (m, 1H), 3.98-3.89 (m, 1H), 3.47 (s, 3H), 3.09- 2.89 (m, 2H), 2.68-2.54 (m, 2H), 2.44-2.35 (m, 1H), 2.32-2.20 (m, 2H), 1.96-1.82 (m, 1H), 1.14 (s, 9H). | m/z 539.4 [M + H]⁺; (column: ChiralPak AD (150 mm * 4.6 mm, 3 um); mobile phase: [0.05% DEA iPrOH]; B%: 5-40%); Rt = 3.761 min 99% ee |
| 443 | 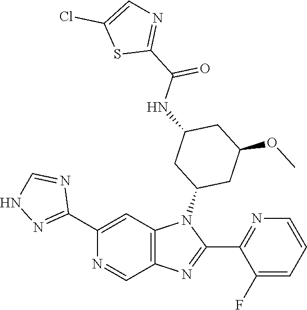 [SMILES: CO[C@@H]1C[C@@H](NC(=O)c2ncc(Cl)s2)C[C@@H](n2c(-c3ncccc3F)nc3cnc(-c4nc[nH]n4)cc32)C1] | (400 MHz, CDCl₃) δ 9.25 (s, 1 H), 8.67-8.66 (m, 1 H), 8.53 (s, 1 H), 8.19 (s, 1 H), 7.78-7.69 (m, 1 H), 7.66 (s, 1 H), 7.62-7.53 (m, 1 H), 7.26-7.25 (m, 1 H), 5.26- 5.20 (m, 1 H), 4.47-4.40 (m, 1 H), 3.89 (br s, 1 H), 3.40 (s, 3 H), 2.57-2.38 (m, 5 H), 1.77-1.71 (m, 1 H). | 554.2 [M + H]⁺; (column: DAICEL CHIRALPAK IG (250 mm * 30 mm, 10 um); mobile phase: [0.1% NH₃H₂O IPA]; B%: 45%-45%, min); Rt = 4.947 min 100% ee |
| 444 | 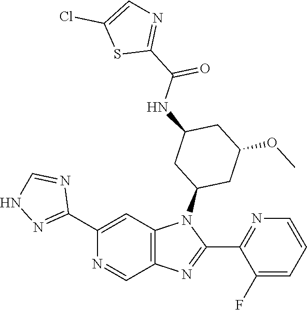 [SMILES: CO[C@H]1C[C@H](NC(=O)c2ncc(Cl)s2)C[C@H](n2c(-c3ncccc3F)nc3cnc(-c4nc[nH]n4)cc32)C1] | (400 MHz, CDCl₃) δ 9.25 (s, 1 H), 8.67-8.66 (m, 1 H), 8.53 (s, 1 H), 8.18 (s, 1 H), 7.77-7.70 (m, 1 H), 7.66 (s, 1 H), 7.63-7.54 (m, 1 H), 7.26- 7.24 (m, 1 H), 5.27-5.21 (m, 1 H), 4.48-4.42 (m, 1 H), 3.89 (br s, 1 H), 3.40 (s, 3 H), 2.57-2.40 (m, 5 H), 1.74- 1.67 (m, 1 H). | 554.2 [M + H]⁺; (column: DAICEL CHIRALPAK IG (250 mm * 30 mm, 10 um); mobile phase: [0.1% NH₃H₂O IPA]; B%: 45%-45%, min); Rt = 5.719 min 100% ee |
| 445 | 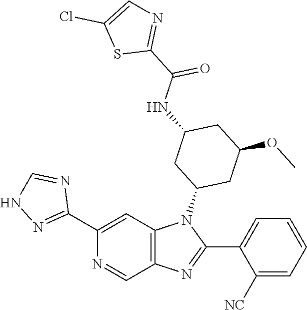 [SMILES: CO[C@@H]1C[C@@H](NC(=O)c2ncc(Cl)s2)C[C@@H](n2c(-c3ccccc3C#N)nc3cnc(-c4nc[nH]n4)cc32)C1] | (400 MHz, CDCl₃) δ 9.13 (s, 1 H), 8.42 (s, 1 H), 8.09 (s, 1 H), 7.88-7.87 (m, 1 H), 7.81- 7.75 (m, 1 H), 7.72-7.67 (m, 1 H), 7.60-7.55 (m, 2 H), 7.14-7.12 (m, 1 H), 4.66- 4.56 (m, 1 H), 4.26 (br s, 1 H), 3.77 (br s, 1 H), 3.20 (s, 3 H), 2.40-2.23 (m, 5 H), 1.59-1.54 (m, 1 H). | 560.2 [M + H]⁺; (column: DAICEL CHIRALPAK IG (250 mm * 30 mm, 10 um); mobile phase: [0.1% NH₃H₂O IPA]; B%: 45%-45%, min); Rt = 5.064 min 100% ee |
| 446 | 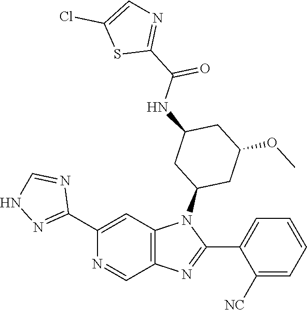 [SMILES: CO[C@H]1C[C@H](NC(=O)c2ncc(Cl)s2)C[C@H](n2c(-c3ccccc3C#N)nc3cnc(-c4nc[nH]n4)cc32)C1] | (400 MHz, CDCl₃) δ 9.15 (s, 1 H), 8.47 (s, 1 H), 8.12 (s, 1 H), 7.89-7.87 (m, 1 H), 7.81- 7.75 (m, 1 H), 7.73-7.66 (m, 1 H), 7.61-7.53 (m, 2 H), 7.18-7.16 (m, 1 H), 4.62- 4.56 (m, 1 H), 4.27 (br s, 1 H), 3.77 (br s, 1 H), 3.20 (s, 3 H), 2.40-2.29 (m, 5 H), 1.62-1.56 (m, 1 H). | 560.2 [M + H]⁺; (column: DAICEL CHIRALPAK IG (250 mm * 30 mm, 10 um); mobile phase: [0.1% NH₃H₂O IPA]; B%: 45%-45%, min); Rt = 5.534 min 91.0% |
| 447 | 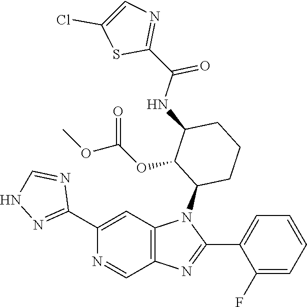 [SMILES: COC(=O)O[C@@H]1[C@@H](NC(=O)c2ncc(Cl)s2)CCC[C@H]1n1c(-c2ccccc2F)nc2cnc(-c3nc[nH]n3)cc21] | (400 MHz, DMSO-d₆) δ 14.71 (s, 1H), 9.19-9.02 (m, 2H), 8.53 (s, 1H), 8.18-8.07 (m, 1H), 8.02 (s, 1H), 7.80- 7.69 (m, 1H), 7.61 (t, 1H), 7.56-7.45 (m, 2H), 5.58 (t, 1H), 4.47-4.31 (m, 1H), 4.23-4.10 (m, 1H), 3.26 (s, 3H), 2.48-2.40 (m, 1H), 2.28-2.20 (m, 1H), 2.06-1.75 (m, 3H), 1.70-1.56 (m, 1H). | m/z 597.3 [M + H]⁺ (column: ChiralPak IH (100 mm * 4.6 mm, 5 um); mobile phase: [0.05% DEA MeOH]; B%: 8 min @ 30%); Rt = 0.885 min 100% ee |
| 451 | 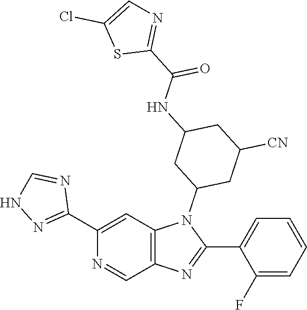 [SMILES: N#CC1CC(NC(=O)c2ncc(Cl)s2)CC(n2c(-c3ccccc3F)nc3cnc(-c4nc[nH]n4)cc32)C1] | (400 MHz, CDCl₃) δ 9.21 (s, 1 H), 8.57 (s, 1 H), 8.21 (s, 1 H), 7.70-7.57 (m, 4 H), 7.45- 7.43 (m, 1 H), 7.35-7.31 (m, 2 H), 5.08 (br s, 1 H), 4.31- 4.13 (m, 2 H), 2.80-2.38 (m, 6 H), 2.33-2.20 (m, 7 H), 2.03-1.97 (m, 1 H). | m/z 548.1 [M + H]⁺ |
| | (relative stereochemistry at | | |
| | centers 1 and 5 of the | | |
| | cyclohexyl ring cis with | | |
| | stereochemistry unassigned at | | |
| | center 3) | | |
| 456 | 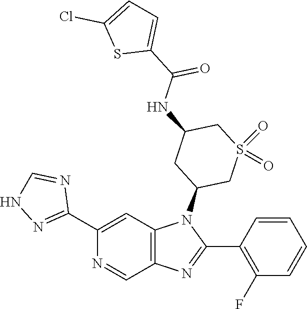 [SMILES: O=C(N[C@@H]1C[C@H](n2c(-c3ccccc3F)nc3cnc(-c4nc[nH]n4)cc32)CS(=O)(=O)C1)c1ccc(Cl)s1] | (400 MHz, DMSO-d₆) δ 9.14 (s, 1H), 8.81 (s, 1H), 8.11 (s, 1H), 7.69-7.57 (m, 2H), 7.55-7.40 (m, 1H), 7.32-7.23 (m, 2H), 7.08 (d, 1H), 6.99 (t, 1H), 4.79 (t, 1H), 4.72- 4.63 (m, 1H), 4.47 (t, 1H), 4.07 (dd, 1H), 3.78 (d, 1H), 3.50 (d, 1H), 2.89-2.76 (m, 1H), 2.37 (d, 1H). | m/z 572.0 [M + H]⁺ (column: ColumnTek Enantiocel A5-5 (250 mm * 30 mm); mobile phase: [0.05% NH₄OH EtOH]; B%: 40%); Rt = 8.41 min 99% ee |
| 457 | 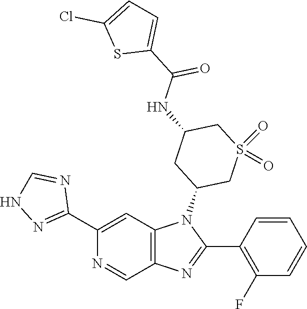 [SMILES: O=C(N[C@H]1C[C@@H](n2c(-c3ccccc3F)nc3cnc(-c4nc[nH]n4)cc32)CS(=O)(=O)C1)c1ccc(Cl)s1] | (400 MHz, DMSO-d₆) δ 9.14 (s, 1H), 8.81 (s, 1H), 8.11 (s, 1H), 7.69-7.57 (m, 2H), 7.55-7.40 (m, 1H), 7.32-7.23 (m, 2H), 7.08 (d, 1H), 6.99 (t, 1H), 4.79 (t, 1H), 4.72- 4.63 (m, 1H), 4.47 (t, 1H), 4.07 (dd, 1H), 3.78 (d, 1H), 3.50 (d, 1H), 2.89-2.76 (m, 1H), 2.37 (d, 1H). | m/z 572.0 [M + H]⁺ (column: ColumnTek Enantiocel A5-5 (250 mm * 30 mm); mobile phase: [0.05% NH₄OH EtOH]; B%: 40%); Rt = 6.56 min 99% ee |
| 458 | 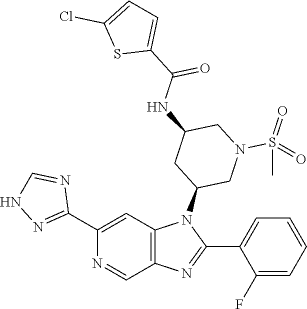 [SMILES: CS(=O)(=O)N1C[C@H](NC(=O)c2ccc(Cl)s2)C[C@H](n2c(-c3ccccc3F)nc3cnc(-c4nc[nH]n4)cc32)C1] | (400 MHz, CDCl₃) δ 9.16 (s, 1 H), 8.50 (s, 1 H), 8.16 (s, 1 H), 7.72-7.62 (m, 2 H), 7.46-7.38 (m, 2 H), 7.37- 7.31 (m, 1 H), 6.88 (s, 2 H), 4.48 (s, 1 H), 4.32 (s, 1 H), 4.08 (s, 2 H), 3.58-3.42 (m, 1 H), 2.86 (s, 3 H), 2.82-2.73 (m, 1 H), 2.66-2.51 (m, 1 H), 2.49-2.38 (m, 1 H). | m/z 601.2 [M + H]⁺; (column: DAICEL CHIRALPAK IC (250 mm * 30 mm, 10 um); mobile phase: [0.1% NH₃H₂O EtOH]; B%: 50%-50%, min); Rt = 2.534 min 98.7% ee |
| 459 | 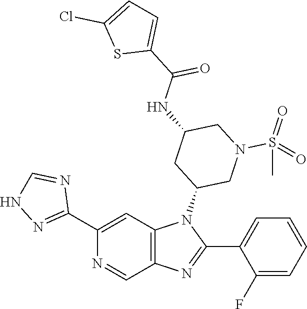 [SMILES: CS(=O)(=O)N1C[C@@H](NC(=O)c2ccc(Cl)s2)C[C@@H](n2c(-c3ccccc3F)nc3cnc(-c4nc[nH]n4)cc32)C1] | (400 MHz, CDCl₃) δ 9.15 (s, 1 H), 8.46 (s, 1 H), 8.27-8.05 (m, 1 H), 7.71-7.63 (m, 2 H), 7.48-7.40 (m, 2 H), 7.37- 7.30 (m, 1 H), 6.88 (s, 2 H), 4.47 (s, 1 H), 4.31 (s, 1 H), 4.08 (s, 2 H), 3.55-3.41 (m, 1 H), 2.85 (s, 3 H), 2.80-2.69 (m, 1 H), 2.60-2.49 (m, 1 H), 2.44 (br s, 1 H). | m/z 601.1 [M + H]⁺; (column: DAICEL CHIRALPAK IC (250 mm * 30 mm, 10 um); mobile phase: [0.1% NH₃H₂O EtOH]; B%: 50%-50%, min); Rt = 3.812 min 97.0% |
| 460 | 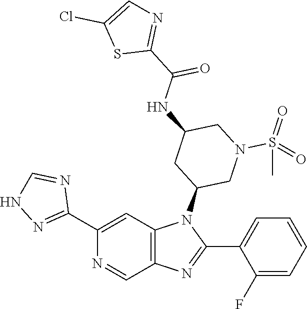 [SMILES: CS(=O)(=O)N1C[C@H](NC(=O)c2ncc(Cl)s2)C[C@H](n2c(-c3ccccc3F)nc3cnc(-c4nc[nH]n4)cc32)C1] | (400 MHz, CDCl₃) δ 9.20 (s, 1 H), 8.53 (s, 1 H), 8.16 (s, 1 H), 7.75-7.66 (m, 3 H), 7.48- 7.43 (m, 1 H), 7.41-7.33 (m, 2 H), 4.60-4.50 (m, 1 H), 4.42-4.30 (m, 1 H), 4.23- 4.10 (m, 2 H), 3.64-3.52 (m, 1 H), 3.03-2.94 (m, 1 H), 2.91 (s, 3 H), 2.74-2.63 (m, 1 H), 2.60-2.52 (m, 1 H) | m/z 602.1 [M + H]⁺; (column: DAICEL CHIRALPAK IG (250 mm * 30 mm, 10 um); mobile phase: [0.1% NH₃H₂O EtOH]; B%: 45%-45%, min); Rt = 2.418 min 99.8% ee |
| 461 | 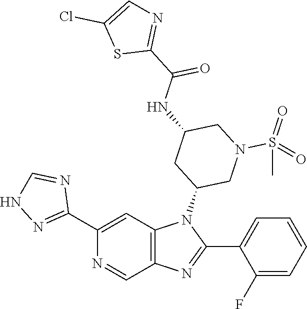 [SMILES: CS(=O)(=O)N1C[C@@H](NC(=O)c2ncc(Cl)s2)C[C@@H](n2c(-c3ccccc3F)nc3cnc(-c4nc[nH]n4)cc32)C1] | (400 MHz, CDCl₃) δ 9.11 (s, 1 H), 8.49 (s, 1 H), 8.10 (s, 1 H), 7.67-7.56 (m, 3 H), 7.40- 7.32 (m, 2 H), 7.31-7.24 (m, 1 H), 4.53-4.37 (m, 1 H), 4.26 (s, 1 H), 4.15-4.01 (m, 2 H), 3.55-3.45 (m, 1 H), 2.96- 2.88 (m, 1 H), 2.82 (s, 3 H), 2.67-2.56 (m, 1 H), 2.49- 2.41 (m, 1 H). | m/z 602.1 [M + H]⁺; (column: DAICEL CHIRALPAK IG (250 mm * 30 mm, 10 um); mobile phase: [0.1% NH₃H₂O EtOH]; B%: 45%-45%, min); Rt = 3.746 min 98.5% ee |
| 462 | 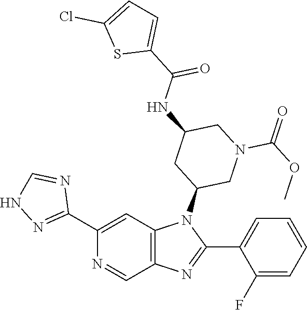 [SMILES: COC(=O)N1C[C@H](NC(=O)c2ccc(Cl)s2)C[C@H](n2c(-c3ccccc3F)nc3cnc(-c4nc[nH]n4)cc32)C1] | (400 MHz, CDCl₃) δ 9.16 (s, 1 H), 8.52 (br s, 1 H), 8.17 (br s, 1 H), 7.68-7.65 (m, 2 H), 7.42-7.38 (m, 2 H), 7.32- 7.30 (m, 1 H), 6.89-6.88 (m, 1 H), 6.68 (br s, 1 H), 4.46- 4.41 (m, 2 H), 4.36-4.30 (m, 1 H), 4.25-4.18 (m, 1 H), 3.68 (s, 3 H), 3.49 (br s, 1 H), 2.85-2.78 (m, 1 H), 2.60- 2.53 (m, 1 H), 2.48-2.44 (br s, 1 H). | m/z 581.2 [M + H]⁺; (column: DAICEL CHIRALPAK IG (250 mm * 30 mm, 10 um); mobile phase: [0.1% NH₃H₂O EtOH]; B%: 40%-40%, min); Rt = 1.485 min 100% ee |
| 463 | 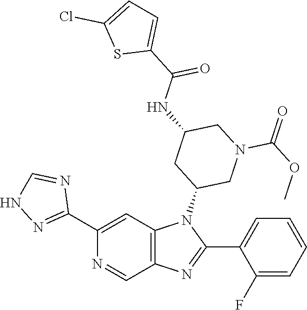 [SMILES: COC(=O)N1C[C@@H](NC(=O)c2ccc(Cl)s2)C[C@@H](n2c(-c3ccccc3F)nc3cnc(-c4nc[nH]n4)cc32)C1] | (400 MHz, CDCl₃) δ 9.15 (s, 1 H), 8.49 (s, 1 H), 8.16 (s, 1 H), 7.68-7.65 (m, 2 H), 7.42- 7.38 (m, 2 H), 7.32-7.30 (m, 1 H), 6.89-6.88 (m, 1 H), 6.69 (br s, 1 H), 4.47-4.40 (m, 2 H), 4.35-4.29 (m, 1 H), 4.20 (br s, 1 H), 3.68 (s, 3 H), 3.51-3.43 (m, 1 H), 2.81- 2.75 (m, 1 H), 2.58-2.52 (m, 1 H), 2.47-2.42 (br s, 1 H). | m/z 581.2 [M + H]⁺; (column: DAICEL CHIRALPAK IG (250 mm * 30 mm, 10 um); mobile phase: [0.1% NH₃H₂O EtOH]; B%: 40%-40%, min); Rt = 2.171 min 98.7% ee |
| 464 | 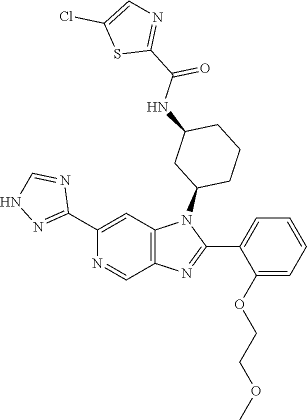 [SMILES: COCCOc1ccccc1-c1nc2cnc(-c3nc[nH]n3)cc2n1[C@@H]1CCC[C@H](NC(=O)c2ncc(Cl)s2)C1] | (400 MHz, DMSO-d₆) δ 14.68 (s, 1H), 9.18 (s, 1H), 9.08 (s, 1H), 8.45 (s, 1H), 8.15-7.99 (m, 2H), 7.62 (t, 1H), 7.49 (d, 1H), 7.32 (d, 1H), 7.16 (t, 1H), 4.38-4.00 (m, 3H), 3.99-3.72 (m, 1H), 3.52 (s, 2H), 3.10 (s, 3H), 2.73-2.56 (m, 1H), 2.41-1.72 (m, 5H), 1.70-1.54 (m, 1H), 1.48so1.24 (m, 1H). | m/z 579.0 [M + H]⁺ |

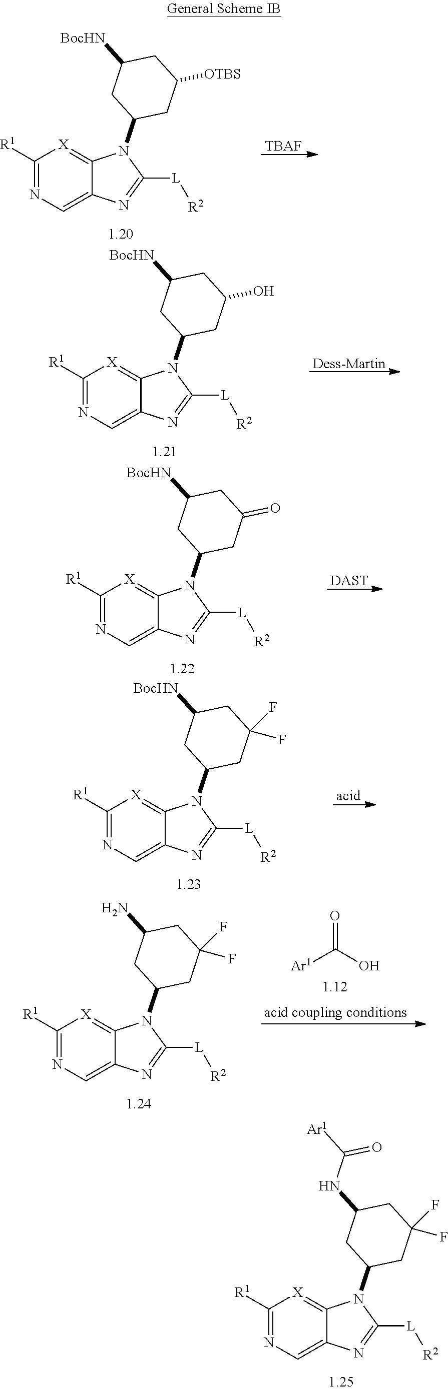

Compounds of formula 1.25 were obtained through General Scheme IB (relative stereochemistry is indicated in this General Scheme). Beginning with a compound of formula 1.20, the TBS protecting group (or other suitable hydroxyl protecting group) of a compound of formula 1.20 is removed with TBAF or other suitable deprotecting conditions to afford compounds of formula 1.21. The hydroxyl moiety of a compound of formula 1.21 is oxidized to a ketone moiety via reaction with the Dess-Martin reagent or other suitable oxidation agent to afford a compound of formula 1.22. The compound of formula 1.22 was then fluorinated with DAST or other suitable fluorination agent to afford compounds of formula 1.23. The Boc protecting group of a compound of formula 1.23 was then removed under acidic conditions such as TFA or HCl to form a compound of formula 1.24. Ar¹ groups were then installed by reacting an Ar¹ carboxylic acid of formula 1.12 or a basic salt (i.e., Li, K, or Na) thereof with the amine of formula 1.24 using acid coupling conditions known in the art, such as using one of the following reagents-HATU, EDCI, HOBt-along with a base, such as DIPEA (Hunig's base), pyridine, or TEA. If the compounds still contain any protecting groups on the Cy or R² groups, they may be removed under acidic conditions such as hydrochloric acid to afford compounds of formula 1.25.

Preparation of Examples 453 and 454 Via General Scheme IB (relative stereochemistry is indicated for Steps A-E)

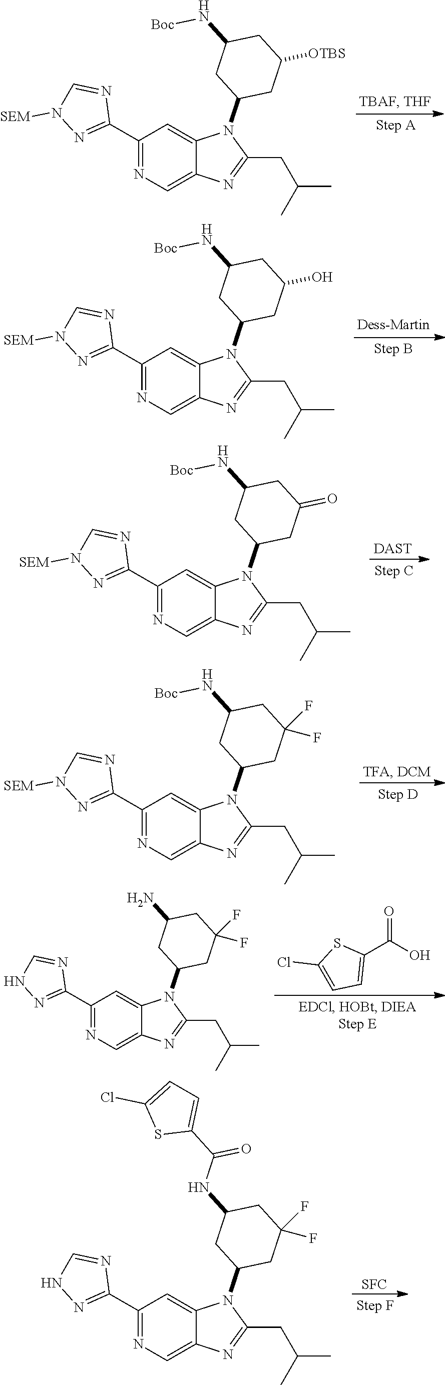

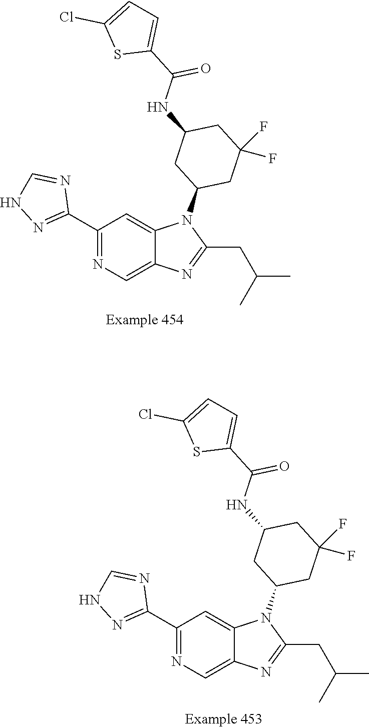 [SMILES: CC(C)Cc1nc2cnc(-c3nc[nH]n3)cc2n1[C@H]1C[C@@H](NC(=O)c2ccc(Cl)s2)CC(F)(F)C1 ; CC(C)Cc1nc2cnc(-c3nc[nH]n3)cc2n1[C@@H]1C[C@H](NC(=O)c2ccc(Cl)s2)CC(F)(F)C1]

### Step A. Synthesis of tert-butyl (3-hydroxy-5-(2-isobutyl-6-(1-((2-(trimethylsilyl)ethoxy)methyl)-1H-1,2,4-triazol-3-yl)-1H-imidazo[4,5-c]pyridin-1-yl)cyclohexyl)carbamate

To a solution of tert-butyl (3-((tert-butyldimethylsilyl)oxy)-5-(2-isobutyl-6-(1-((2-(trimethylsilyl)ethoxy)methyl)-1H-1,2,4-triazol-3-yl)-1H-imidazo[4,5-c]pyridin-1-yl)cyclohexyl)carbamate (0.14 g, 200 μmol) in THF (5 mL) was added TBAF (400 μL, 1 M in THF). After the addition was complete, the reaction mixture was stirred at rt for 2 hrs and concentrated to dryness. The residue was purified by column chromatography on silica gel to give tert-butyl (3-hydroxy-5-(2-isobutyl-6-(1-((2-(trimethylsilyl)ethoxy)methyl)-1H-1,2,4-triazol-3-yl)-1H-imidazo[4,5-c]pyridin-1-yl)cyclohexyl)carbamate (0.1 g, 62.6% yield). LC-MS: m/z 586.4 [(M+1)⁺].

### Step B. Synthesis of tert-butyl (3-(2-isobutyl-6-(1-((2-(trimethylsilyl)ethoxy)methyl)-1H-1,2,4-triazol-3-yl)-1H-imidazo[4,5-c]pyridin-1-yl)-5-oxocyclohexyl)carbamate

To a solution of tert-butyl N-[3-hydroxy-5-[2-isobutyl-6-[4-(2-trimethylsilylethoxymethyl)-1,2,4-triazol-3-yl]imidazo[4,5-c]pyridin-1-yl]cyclohexyl]carbamate (0.1 g, 171 μmol) in DCM (5 mL) was added Dess-Martin reagent (145 mg, 341 μmol). After the addition was complete, the reaction mixture was stirred at rt for 16 hrs. Sat. aq. Na₂SO₃ solution (25 mL) was added, and the aqueous portion was extracted with DCM (3×25 mL). The combined organic layers were washed with brine (25 mL), dried over Na₂SO₄, filtered, and concentrated to dryness. The residue was purified by column chromatography on silica gel to give tert-butyl (3-(2-isobutyl-6-(1-((2-(trimethylsilyl)ethoxy)methyl)-1H-1,2,4-triazol-3-yl)-1H-imidazo[4,5-c]pyridin-1-yl)-5-oxocyclohexyl)carbamate (70 mg, 49.2% yield). LC-MS: m/z 584.4 [(M+1)⁺].

### Step C. Synthesis of tert-butyl (3,3-difluoro-5-(2-isobutyl-6-(1-((2-(trimethylsilyl)ethoxy)methyl)-1H-1,2,4-triazol-3-yl)-1H-imidazo[4,5-c]pyridin-1-yl)cyclohexyl)carbamate

To a solution of tert-butyl (3-(2-isobutyl-6-(1-((2-(trimethylsilyl)ethoxy)methyl)-1H-1,2,4-triazol-3-yl)-1H-imidazo[4,5-c]pyridin-1-yl)-5-oxocyclohexyl)carbamate (70 mg, 120 μmol) in DCM (5 mL) was added N-ethyl-N-(trifluoro-λ4-sulfanyl)ethanamine (48.3 mg, 300 μmol) at 0° C. under nitrogen. After the addition was complete, the reaction mixture was allowed to warm to rt and stirred at rt for 16 hrs. Sat. aq. NaHCO₃ solution (20 mL) was added, and the aqueous portion extracted with DCM (5×15 mL). The combined organic layers were washed with brine (20 mL), dried over Na₂SO₄, filtered, and concentrated to dryness. The residue was purified by column chromatography on silica gel to give tert-butyl (3,3-difluoro-5-(2-isobutyl-6-(1-((2-(trimethylsilyl)ethoxy)methyl)-1H-1,2,4-triazol-3-yl)-1H-imidazo[4,5-c]pyridin-1-yl)cyclohexyl)carbamate (55 mg, 69.3% yield). LC-MS: m/606.3 [(M+1)⁺].

### Step D. Synthesis of 3,3-difluoro-5-(2-isobutyl-6-(1H-1,2,4-triazol-3-yl)-1H-imidazo[4,5-c]pyridin-1-yl)cyclohexan-1-amine

To a solution of tert-butyl N-[3,3-difluoro-5-[2-isobutyl-6-[4-(2-trimethylsilylethoxymethyl)-1,2,4-triazol-3-yl]imidazo[4,5-c]pyridin-1-yl]cyclohexyl]carbamate (55 mg, 90.8 μmol) in DCM (1 mL) was added TFA (921 mg, 8.07 mmol, 598 μL). After the addition was complete, the reaction mixture was stirred at rt under nitrogen for 2 hrs and then concentrated to dryness to give 3,3-difluoro-5-(2-isobutyl-6-(1H-1,2,4-triazol-3-yl)-1H-imidazo[4,5-c]pyridin-1-yl)cyclohexan-1-amine (55 mg, HCl salt), which was used without any further purification. LC-MS: m/z 376.1 [(M+1)⁺].

### Step E. Synthesis of 5-chloro-N-(3,3-difluoro-5-(2-isobutyl-6-(1H-1,2,4-triazol-3-yl)-1H-imidazo[4,5-c]pyridin-1-yl)cyclohexyl)thiophene-2-carboxamide

To a solution of 3,3-difluoro-5-[2-isobutyl-6-(1H-1,2,4-triazol-3-yl)imidazo[4,5-c]pyridin-1-yl]cyclohexanamine (55 mg, 147 μmol, HCl salt), 5-chlorothiophene-2-carboxylic acid (23.8 mg, 147 μmol), DIPEA (37.9 mg, 293 μmol, 51.0 μL) and HOBt (29.7 mg, 220 μmol) in DMF (1 mL) was added EDCI (42.1 mg, 220 μmol). After the addition was complete, the reaction mixture was stirred at rt for 16 hrs. The reaction mixture was filtered, and the filtrate was purified by prep-HPLC (column: Boston Green ODS 150*30 mm*5 um; mobile phase: [water (FA)-ACN]; B %: 33%-63%, 6 min) to give 5-chloro-N-(3,3-difluoro-5-(2-isobutyl-6-(1H-1,2,4-triazol-3-yl)-1H-imidazo[4,5-c]pyridin-1-yl)cyclohexyl)thiophene-2-carboxamide (9.0 mg, 11.3% yield). LC-MS: m/z 520.1 [(M+1)⁺]

### Step F. Synthesis of 5-chloro-N-((1R,5S)-3,3-difluoro-5-(2-isobutyl-6-(1H-1,2,4-triazol-3-yl)-1H-imidazo[4,5-c]pyridin-1-yl)cyclohexyl)thiophene-2-carboxamide (Example 453) and 5-chloro-N-((1S,5R)-3,3-difluoro-5-(2-isobutyl-6-(1H-1,2,4-triazol-3-yl)-1H-imidazo[4,5-c]pyridin-1-yl)cyclohexyl)thiophene-2-carboxamide (Example 454)

The enantiomers of 5-chloro-N-(3,3-difluoro-5-(2-isobutyl-6-(1H-1,2,4-triazol-3-yl)-1H-imidazo[4,5-c]pyridin-1-yl)cyclohexyl)thiophene-2-carboxamide (12 mg, 23.08 μmol) were separated by SFC (column: DAICEL CHIRALPAK IG (250 mm*30 mm, 10 um); mobile phase: [Neu-IPA]; B %: 35%-35%, min) to give Enantiomer #1 (3.4 mg, 27.8% yield, Rt=2.887 min) and Enantiomer #2 (3.8 mg, 30.82% yield, Rt=3.822 min).

Enantiomer #1 (Example 453): ¹H NMR (400 MHz, CD₃OD) δ 8.99 (s, 1H), 8.57 (s, 1H), 8.25 (br s, 1H), 7.58-7.57 (m, 1H), 7.04-7.03 (m, 1H), 4.75 (br s, 1H), 4.46-4.42 (m, 1H), 3.08-2.89 (m, 3H), 2.60-2.52 (m, 3H), 2.38-2.23 (m, 3H), 1.13-1.08 (m, 6H); LC-MS: m/z 520.2 (M+1)⁺.

Enantiomer #2 (Example 454): ¹H NMR (400 MHz, CD₃OD) δ 8.99 (s, 1H), 8.57 (s, 1H), 8.27 (br s, 1H), 7.58-7.57 (m, 1H), 7.04-7.03 (m, 1H), 4.75 (br s, 1H), 4.46-4.42 (m, 1H), 3.07-2.91 (m, 3H), 2.61-2.52 (m, 3H), 2.36-2.24 (m, 3H), 1.13-1.08 (m, 6H); LC-MS: m/z 520.2 (M+1)⁺.

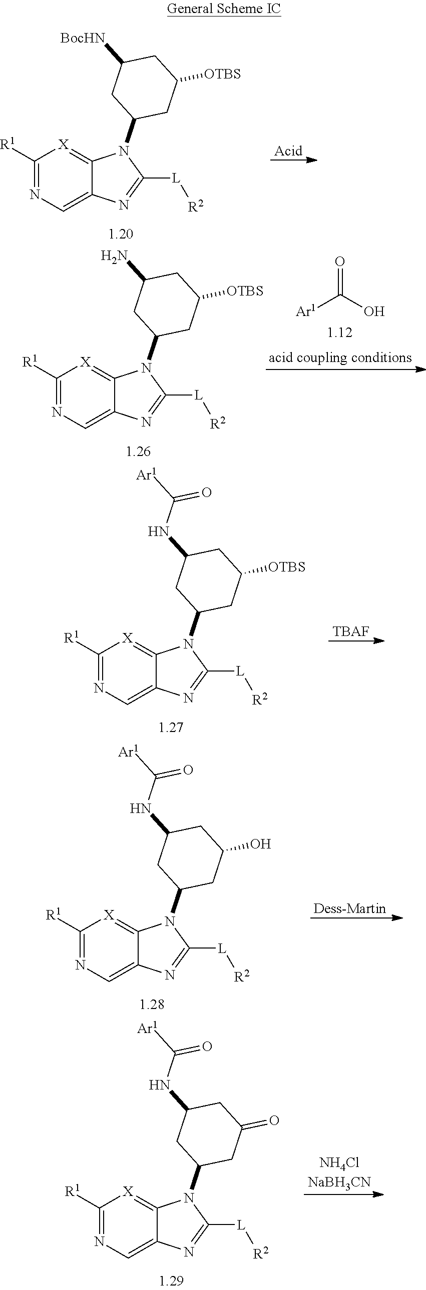

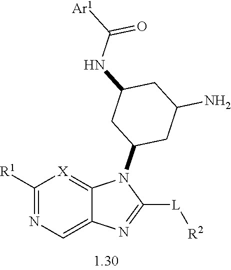

Compounds of formula 1.30 were obtained through General Scheme IC (relative stereochemistry is indicated in this General Scheme). Beginning with a compound of formula 1.20, the Boc protecting group of a compound of formula 1.20 was removed under acidic conditions such as TFA to form a compound of formula 1.26. Ar¹ groups were then installed by reacting an Ar¹ carboxylic acid of formula 1.12 or a basic salt (i.e., Li, K, or Na) thereof with the amine of formula 1.26 using acid coupling conditions known in the art, such as using one of the following reagents—HATU, EDCI, HOBt—along with a base, such as DIPEA (Hunig's base), pyridine, or TEA to afford compounds of formula 1.27. The TBS protecting group of a compound of formula 1.27 was removed with TBAF to afford compounds of formula 1.28. The hydroxyl moiety of a compound of formula 1.28 was oxidized to a ketone moiety via reaction with the Dess-Martin reagent or other suitable oxidation agent to afford a compound of formula 1.29. A compound of formula 1.29 then underwent a reductive animation reaction with ammonium chloride and a reducing agent such as NaBH₃CN to afford compounds of formula 1.30.

### Preparation of Examples 455 Via General Scheme IC (Relative Stereochemistry is Indicated in the Below Scheme)

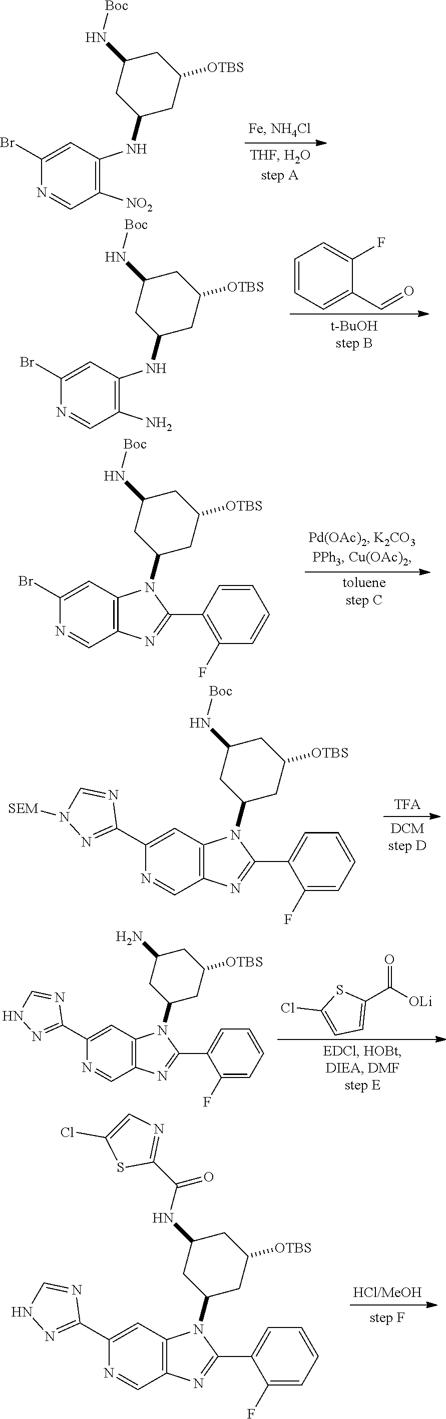

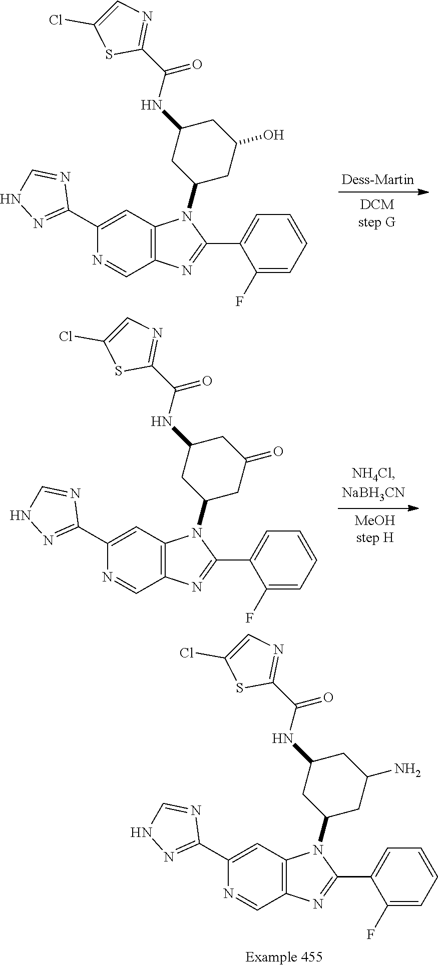

### Step A. Synthesis of tert-butyl (3-((5-amino-2-bromopyridin-4-yl)amino)-5-((tert-butyldimethylsilyl)oxy)cyclohexyl)carbamate

To a solution of tert-butyl (3-((2-bromo-5-nitropyridin-4-yl)amino)-5-((tert-butyldimethylsilyl)oxy)cyclohexyl)carbamate (500 mg, 917 μmol) in THF (6 mL) was added Fe (256 mg, 4.58 mmol) and NH₄Cl (58.8 mg, 1.10 mmol) in H₂O (3 mL). The reaction mixture was stirred at 75° C. for 16 hrs. The reaction mixture was filtered, and the filtrate was concentrated to dryness to give tert-butyl (3-((5-amino-2-bromopyridin-4-yl)amino)-5-((tert-butyldimethylsilyl)oxy)cyclohexyl)carbamate (980 mg), which was used without further purification. LC-MS: m/z 515.1 [M+H]⁺.

### Step B. Synthesis of tert-butyl (3-(6-bromo-2-(2-fluorophenyl)-1H-imidazo[4,5-c]pyridin-1-yl)-5-((tert-butyldimethylsilyl)oxy)cyclohexyl) carbamate

To a solution of tert-butyl (3-((5-amino-2-bromopyridin-4-yl)amino)-5-((tert-butyldimethylsilyl)oxy)cyclohexyl)carbamate (980 mg, 1.90 mol) in t-BuOH (10 mL) was added 2-fluorobenzaldehyde (177 mg, 1.43 mmol). The reaction mixture was stirred at 90° C. for 72 hrs and then concentrated to dryness. The residue was purified by flash silica gel chromatography to give tert-butyl (3-(6-bromo-2-(2-fluorophenyl)-1H-imidazo[4,5-c]pyridin-1-yl)-5-((tert-butyldimethylsilyl)oxy)cyclohexyl) carbamate (650 mg, 50.7% yield). LC-MS: 619.2 [M+H]⁺.

### Step C. Synthesis of tert-butyl (3-((tert-butyldimethylsilyl)oxy)-5-(2-(2-fluorophenyl)-6-(4-((2-(trimethylsilyl)ethoxy)methyl)-1H-1,2,4-triazol-3-yl)-1H-imidazo[4,5-c]pyridin-1-yl)cyclohexyl)carbamate

A mixture of tert-butyl (3-(6-bromo-2-(2-fluorophenyl)-1H-imidazo[4,5-c]pyridin-1-yl)-5-((tert-butyldimethylsilyl)oxy)cyclohexyl) carbamate (650 mg, 1.05 mmol), trimethyl-[2-(1,2,4-triazol-1-ylmethoxy)ethyl]silane (314 mg, 1.58 mmol), K₂CO₃ (290 mg, 2.10 mmol), PPh₃ (138 mg, 525 μmol), Cu(OAc)₂ (38.1 mg, 210 μmol) and Pd(OAc)₂ (11.8 mg, 52.5 μmol) in toluene (10 mL) was degassed and purged with N₂ for 3 times, and then the mixture was stirred at 110° C. for 16 hrs under N₂ atmosphere. The reaction mixture was concentrated to dryness. The residue was purified by flash silica gel chromatography to give tert-butyl (3-((tert-butyldimethylsilyl)oxy)-5-(2-(2-fluorophenyl)-6-(4-((2-(trimethylsilyl)ethoxy)methyl)-1H-1,2,4-triazol-3-yl)-1H-imidazo[4,5-c]pyridin-1-yl)cyclohexyl)carbamate (570 mg, 73.6% yield). LC-MS: m/z 738.2 [M+H]⁺.

### Step D. Synthesis of 3-((tert-butyldimethylsilyl)oxy)-5-(2-(2-fluorophenyl)-6-(1H-1,2,4-triazol-3-yl)-1H-imidazo[4,5-c]pyridin-1-yl)cyclohexan-1-amine

To a solution of tert-butyl (3-((tert-butyldimethylsilyl)oxy)-5-(2-(2-fluorophenyl)-6-(4-((2-(trimethylsilyl)ethoxy)methyl)-1H-1,2,4-triazol-3-yl)-1H-imidazo[4,5-c]pyridin-1-yl)cyclohexyl)carbamate (270 mg, 366 μmol) in DCM (3 mL) was added TFA (1.5 mL). The reaction mixture was stirred at rt for 1 hr and concentrated to dryness to give 3-((tert-butyldimethylsilyl)oxy)-5-(2-(2-fluorophenyl)-6-(1H-1,2,4-triazol-3-yl)-1H-imidazo[4,5-c]pyridin-1-yl)cyclohexan-1-amine (230 mg, TFA salt), which was used without further purification. LC-MS: m/z 508.2 [M+H]⁺.

### Step E. Synthesis of N-(3-((tert-butyldimethylsilyl)oxy)-5-(2-(2-fluorophenyl)-6-(1H-1,2,4-triazol-3-yl)-1H-imidazo[4,5-c]pyridin-1-yl)cyclohexyl)-5-chlorothiazole-2-carboxamide

To a solution of 3-((tert-butyldimethylsilyl)oxy)-5-(2-(2-fluorophenyl)-6-(1H-1,2,4-triazol-3-yl)-1H-imidazo[4,5-c]pyridin-1-yl)cyclohexan-1-amine (230 mg, 370 μmol, TFA salt) and (5-chlorothiazole-2-carbonyl)oxylithium (81.5 mg, 481 μmol) in DMF (3 mL) was added EDCI (142 mg, 740 μmol), HOBt (100 mg, 740 μmol) and DIPEA (239 mg, 1.85 mmol). The mixture was stirred at rt for 16 hrs. To the reaction mixture was added H₂O (5 mL), and the aqueous portion extracted with EtOAc (12 mL×2). The combined organic layers were washed with brine (6 mL×2), dried over Na₂SO₄, filtered, and concentrated to dryness. The residue was purified by flash silica gel chromatography to give N-(3-((tert-butyldimethylsilyl)oxy)-5-(2-(2-fluorophenyl)-6-(1H-1,2,4-triazol-3-yl)-1H-imidazo[4,5-c]pyridin-1-yl)cyclohexyl)-5-chlorothiazole-2-carboxamide (160 mg, 60.9% yield). LC-MS: m/z 653.3 [M+H]⁺.

### Step F. Synthesis of 5-chloro-N-(3-(2-(2-fluorophenyl)-6-(1H-1,2,4-triazol-3-yl)-1H-imidazo[4,5-c]pyridin-1-yl)-5-hydroxycyclohexyl)thiazole-2-carboxamide

A mixture of N-(3-((tert-butyldimethylsilyl)oxy)-5-(2-(2-fluorophenyl)-6-(1H-1,2,4-triazol-3-yl)-1H-imidazo[4,5-c]pyridin-1-yl)cyclohexyl)-5-chlorothiazole-2-carboxamide (150 mg, 230 μmol) in HCl/MeOH (2 mL, 4 M) was stirred at rt for 2 hrs. The reaction mixture was concentrated to dryness. To the residue was added MeOH (3 mL). The solution was cooled to 0° C. and adjusted to pH 9 with the addition of sat. aq. Na₂CO₃ solution. The aqueous portion was extracted with DCM (15 mL×2). The combined organic layers were washed with brine (5 mL), dried over Na₂SO₄, filtered, and concentrated to dryness to give 5-chloro-N-(3-(2-(2-fluorophenyl)-6-(1H-1,2,4-triazol-3-yl)-1H-imidazo[4,5-c]pyridin-1-yl)-5-hydroxycyclohexyl)thiazole-2-carboxamide (124 mg), which was used without further purification. LC-MS: m/z 539.2 [M+H]⁺.

### Step G. Synthesis of 5-chloro-N-(3-(2-(2-fluorophenyl)-6-(1H-1,2,4-triazol-3-yl)-1H-imidazo[4,5-c]pyridin-1-yl)-5-oxocyclohexyl)thiazole-2-carboxamide

To a solution of 5-chloro-N-(3-(2-(2-fluorophenyl)-6-(1H-1,2,4-triazol-3-yl)-1H-imidazo[4,5-c]pyridin-1-yl)-5-hydroxycyclohexyl)thiazole-2-carboxamide (114 mg, 212 μmol) in DCM (5 mL) was added Dess-Martin reagent (179 mg, 423 μmol). The reaction mixture was stirred at rt for 48 hrs. Water (6 mL) was added to the reaction mixture, and the reaction mixture was cooled to 0° C. and adjusted to pH 8 by the addition of sat. aq. NaHCO₃ solution. The aqueous portion was extracted with DCM (10 mL×3). The combined organic layers were dried over Na₂SO₄, filtered, and concentrated to dryness. The residue was purified by flash silica gel chromatography to give 5-chloro-N-(3-(2-(2-fluorophenyl)-6-(1H-1,2,4-triazol-3-yl)-1H-imidazo[4,5-c]pyridin-1-yl)-5-oxocyclohexyl)thiazole-2-carboxamide (130 mg). LC-MS: m/z 536.9 [M+H]⁺.

### Step H. Synthesis of N-(3-amino-5-(2-(2-fluorophenyl)-6-(1H-1,2,4-triazol-3-yl)-1H-imidazo[4,5-c]pyridin-1-yl)cyclohexyl)-5-chlorothiazole-2-carboxamide

### Example 455

To a solution of 5-chloro-N-(3-(2-(2-fluorophenyl)-6-(1H-1,2,4-triazol-3-yl)-1H-imidazo[4,5-c]pyridin-1-yl)-5-oxocyclohexyl)thiazole-2-carboxamide (130 mg, 261 μmol) in MeOH (2 mL) was added NH₄Cl (139 mg, 2.61 mmol) and NaBH₃CN (49.2 mg, 782 μmol). The reaction mixture was stirred at rt for 4 hrs, filtered, and concentrated to dryness. The residue was purified by prep-HPLC to give Example 455 (4.8 mg, 3.1% yield) (relative stereochemistry at centers 1 and 5 of the cyclohexyl ring is cis with the stereochemistry unassigned at center 3). ¹H NMR (400 MHz, CD₃OD) δ 9.12 (s, 1H), 8.61 (s, 1H), 8.39 (s, 1H), 7.82 (s, 1H), 7.78-7.71 (m, 2H), 7.51-7.42 (m, 2H), 4.50 (br s, 1H), 4.13 (br s, 1H), 3.26 (br s, 1H), 2.61-2.52 (m, 1H), 2.42-2.28 (m, 4H), 1.78-1.69 (m, 1H); LC-MS: 538.1 m/z [M+H]⁺.

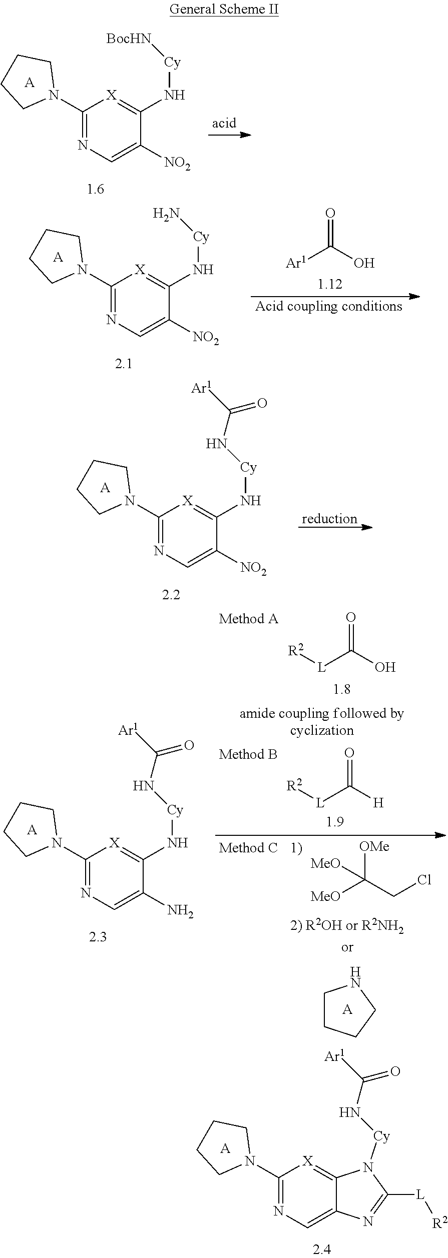

Compounds of formula 2.4 were obtained through General Scheme II. The Boc protecting group of intermediates of formula 1.6 (prepared via General Scheme I) was removed under acidic conditions to generate compounds of formula 2.1. Ar¹ groups were then installed by reacting an Ar¹ carboxylic acid of formula 1.12 or a basic salt (i.e., Li, K, or Na) thereof with the amine of formula 2.1 using acid coupling conditions known in the art, such as using one of the following reagents—HATU, EDCI, HOBt—along with a base, such as DIPEA (Hunig's base) or TEA to afford compounds of formula 2.2. The nitro group in compounds of formula 2.2 was reduced with a reducing reagent such as iron in the presence of ammonium chloride or Pd/C to generate the pyridine-diamine compound of formula 2.3. Subsequently, various L and R² groups were installed by coupling the pyridine-diamine compound of formula 2.3 with acid of formula 1.8 to form an amide intermediate (Method A), which was subsequently cyclized under either basic or acidic conditions to form compounds of formula 2.4. Alternatively, the pyridine-diamine compound of formula 2.3 was condensed with aldehyde of formula 1.9 (Method B), followed by cyclization and in situ oxidation to afford compounds of formula 2.4. Alternatively, the pyridine-diamine of formula 2.3 could be condensed with 2-chloro-1,1,1-trimethoxyethane to afford the 2-chloromethyl imidazole intermediate (Method C), followed by displacement of the chloride with an R²-alcohol or R²-primary amine or heterocyclic amine (saturated or aromatic) to afford compounds of formula 2.4.

### Preparation of Example 74 Via General Scheme II (Method B)

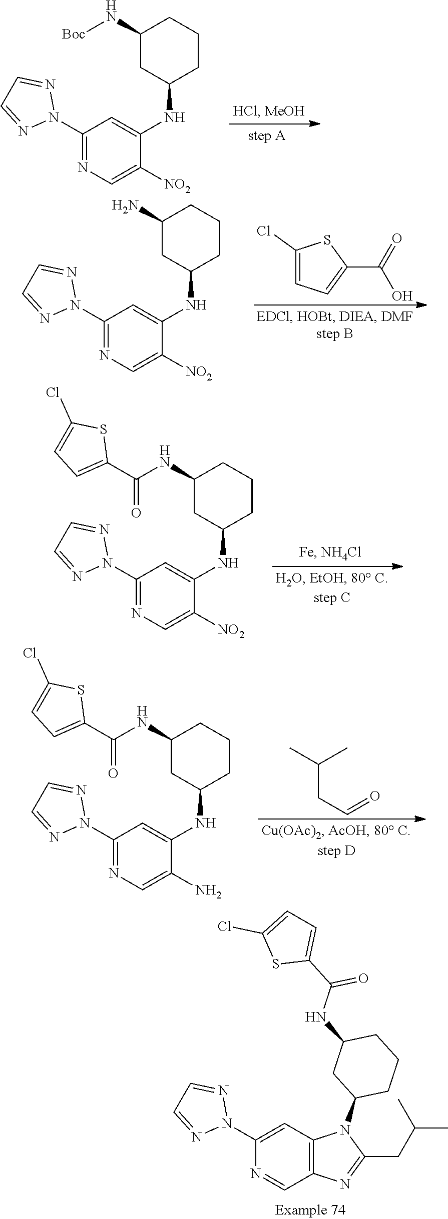

### Step A. Synthesis of (1R,3S)—Ni-(5-nitro-2-(2H-1,2,3-triazol-2-yl)pyridin-4-yl)cyclohexane-1,3-diamine

A solution of tert-butyl N-[(1S,3R)-3-[[5-nitro-2-(triazol-2-yl)-4-pyridyl]amino]cyclohexyl]carbamate (400 mg, 991 μmol) in HCl/MeOH (4 M, 6 mL) was stirred at rt for 1 hr. The reaction mixture was adjusted to pH 7 by the addition of aq. NaHCO₃ solution, and the aqueous portion extracted with EtOAc (50 mL×3). The combined organic layers were concentrated to dryness to give (1R,3S)—Ni-(5-nitro-2-(2H-1,2,3-triazol-2-yl)pyridin-4-yl)cyclohexane-1,3-diamine (300 mg), which was used without further purification. LC-MS: m/z 304.2 [M+H]⁺.

### Step B. Synthesis of 5-chloro-N-((1S,3R)-3-((5-nitro-2-(2H-1,2,3-triazol-2-yl)pyridin-4-yl)amino)cyclohexyl)thiophene-2-carboxamide

To a solution of 5-chlorothiophene-2-carboxylic acid (241 mg, 1.48 mmol) and (1R,3S)—Ni-(5-nitro-2-(2H-1,2,3-triazol-2-yl)pyridin-4-yl)cyclohexane-1,3-diamine (300 mg, 989 μmol) in DMF (3 mL) were added HOBt (200 mg, 1.48 mmol), DIEA (256 mg, 1.98 mmol), and EDCI (284 mg, 1.48 mmol). The reaction mixture was stirred at rt for 16 hrs, then diluted with H₂O (50 mL), and the aqueous portion extracted with EtOAc (50 mL×3). The combined organic layers were washed with brine (10 mL×2), dried over Na₂SO₄, and concentrated to dryness. The residue was purified by flash chromatography on silica gel to give 5-chloro-N-((1S,3R)-3-((5-nitro-2-(2H-1,2,3-triazol-2-yl)pyridin-4-yl)amino)cyclohexyl)thiophene-2-carboxamide (517 mg, 99.0% yield). LC-MS: m/z 448.1 [M+H]⁺.

### Step C. Synthesis of N-((1S,3R)-3-((5-amino-2-(2H-1,2,3-triazol-2-yl)pyridin-4-yl)amino)cyclohexyl)-5-chlorothiophene-2-carboxamide

To a solution of 5-chloro-N-((1S,3R)-3-((5-nitro-2-(2H-1,2,3-triazol-2-yl)pyridin-4-yl)amino)cyclohexyl)thiophene-2-carboxamide (443 mg, 989 μmol) in EtOH (6 mL) and H₂O (1.5 mL) were added Fe (276 mg, 4.95 mmol) and NH₄Cl (265 mg, 4.95 mmol). The reaction mixture was stirred at 80° C. for 1 hr, and then allowed to cool. The resulting slurry was filtered, and the filtrate was concentrated to dryness to give N-((1S,3R)-3-((5-amino-2-(2H-1,2,3-triazol-2-yl)pyridin-4-yl)amino)cyclohexyl)-5-chlorothiophene-2-carboxamide (499 mg), which was used without further purification. LC-MS: m/z 418.1 [M+H]⁺

### Step D. Synthesis of 5-chloro-N-((1S,3R)-3-(2-isobutyl-6-(2H-1,2,3-triazol-2-yl)-1H-imidazo[4,5-c]pyridin-1-yl)cyclohexyl)thiophene-2-carboxamide (Example 74)

To a solution of 3-methylbutanal (24.7 mg, 287 μmol) and N-((1S,3R)-3-((5-amino-2-(2H-1,2,3-triazol-2-yl)pyridin-4-yl)amino)cyclohexyl)-5-chlorothiophene-2-carboxamide (100 mg, 239 μmol) in AcOH (2 mL) was added Cu(OAc)₂·H₂O (47.8 mg, 239 μmol). The reaction mixture was stirred at 80° C. for 3 hrs, and then allowed to cool. The reaction mixture was diluted with H₂O (50 mL) and the aqueous portion extracted with EtOAc (50 mL×3). The combined organic layers were washed with brine (10 mL×2), dried over Na₂SO₄, and concentrated to dryness. The residue was purified by prep-HPLC (column: Phenomenex Gemini-NX C18 75*30 mm*3 um; mobile phase: [water(0.225% FA)-ACN]; B %: 35%-65%, 7 min) to give Example 74 (17 mg, 15% yield). ¹H NMR (400 MHz, CDCl₃) δ 8.93 (s, 1H), 8.11-7.96 (m, 3H), 7.32 (s, 1H), 6.90 (d, 1H), 6.17-6.12 (m, 1H), 4.43-4.37 (m, 1H), 4.27-4.23 (m, 1H), 2.84 (d, 2H), 2.36-2.11 (m, 6H), 2.02 (d, 1H), 1.54-1.44 (m, 2H), 1.10 (dd, 6H). LC-MS: m/z 484.2 [M+H]⁺.

General Scheme II set forth above was used to synthesize the following compounds by using appropriate starting materials:

| Ex. No. | Structure | ¹H NMR | LC-MS and % ee |
|---|---|---|---|
| 17 | 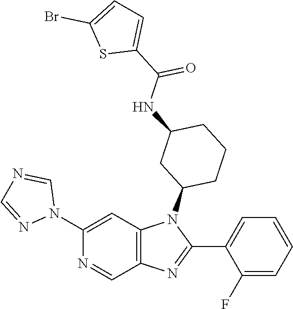 [SMILES: O=C(N[C@H]1CCC[C@@H](n2c(-c3ccccc3F)nc3cnc(-n4cncn4)cc32)C1)c1ccc(Br)s1] | (400 MHZ, CD₃OD) δ 9.43 (s, 1 H), 8.93 (s, 1 H), 8.32 (s, 1 H), 8.27 (s, 1 H), 7.76-7.70 (m, 2 H), 7.49-7.32 (m, 3 H), 7.12 (d, 1 H), 4.31 (t, 1 H), 3.94 (t, 1 H), 2.45-2.20 (m, 2 H), 2.17-2.09 (m, 2 H), 1.57- 1.50 (m, 2 H), 1.33 -1.22 (m, 2 H) | m/z 568.3 [M + H]⁺ |
| 41 | 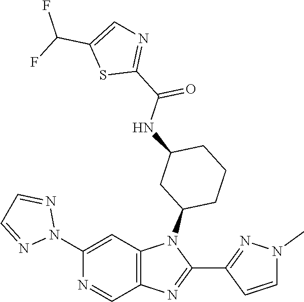 [SMILES: Cn1ccc(-c2nc3cnc(-n4nccn4)cc3n2[C@@H]2CCC[C@H](NC(=O)c3ncc(C(F)F)s3)C2)n1] | (400 MHZ, DMSO-d₆) δ 9.21 (d, 1 H), 8.93 (s, 1 H), 8.34- 8.30 (m, 1 H), 8.27 (s, 1 H), 8.19 (s, 2 H), 7.97 (d, 1 H), 7.62-7.27 (m, 1 H), 6.98 (d, 1 H), 5.82 (br s, 1 H), 4.04 (s, 4 H), 2.66 (q, 1 H), 2.12 (br d, 2 H), 2.04-1.86 (m, 3 H), 1.72-1.51 (m, 2 H) | m/z 525.2 [M + H]⁺ |
| 42 | 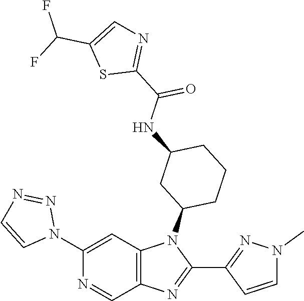 [SMILES: Cn1ccc(-c2nc3cnc(-n4ccnn4)cc3n2[C@@H]2CCC[C@H](NC(=O)c3ncc(C(F)F)s3)C2)n1] | (400 MHZ, DMSO-d₆) δ 9.21 (d, 1 H), 8.96 (s, 1 H), 8.89 (s, 1 H), 8.41 (s, 1 H), 8.32 (s, 1 H), 8.02 (s, 1 H), 7.98 (d, 1 H), 7.61-7.29 (m, 1 H), 6.98 (d, 1 H), 5.83 (br s, 1 H), 4.05 (s, 4 H), 2.74-2.65 (m, 1 H), 2.21-2.11 (m, 2 H), 1.99 (br d, 2 H), 1.90 (br d, 1 H), 1.73-1.53 (m, 2 H) | m/z 525.3 [M + H]⁺ |
| 43 | 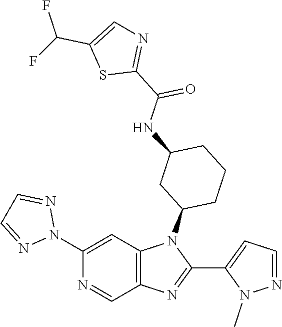 [SMILES: Cn1nccc1-c1nc2cnc(-n3nccn3)cc2n1[C@@H]1CCC[C@H](NC(=O)c2ncc(C(F)F)s2)C1] | (400 MHZ, CD₃OD) δ 8.97 (s, 1 H), 8.51 (s, 1 H), 8.14 (s, 1 H), 8.08 (s, 2 H), 7.75 (d, 1 H), 7.33-7.00 (m, 1 H), 6.84 (d, 1 H), 4.69 (br t, 1 H), 4.06 (s, 4 H), 2.58-2.49 (m, 1 H), 2.39-2.28 (m, 2 H), 2.13-2.04 (m, 3 H), 1.71-1.58 (m, 2 H) | m/z 525.3 [M + H]⁺ |
| 44 | 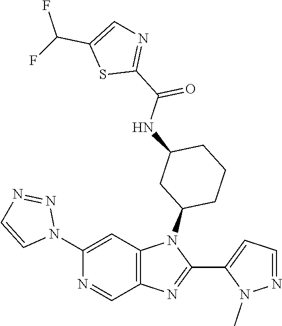 [SMILES: Cn1nccc1-c1nc2cnc(-n3ccnn3)cc2n1[C@@H]1CCC[C@H](NC(=O)c2ncc(C(F)F)s2)C1] | (400 MHZ, CD₃OD) δ 9.00 (s, 1 H), 8.86 (s, 1 H), 8.57 (s, 1 H), 8.14 (s, 1 H), 7.96 (s, 1 H), 7.75 (d, 1 H), 7.33-7.01 (m, 1 H), 6.85 (d, 1 H), 4.75- 4.65 (m, 1 H), 4.06 (s, 4 H), 2.54 (q, 1 H), 2.40-2.28 (m, 2 H), 2.13-2.03 (m, 3 H), 1.73- 1.56 (m, 2 H) | m/z 525.3 [M + H]⁺ |
| 45 | 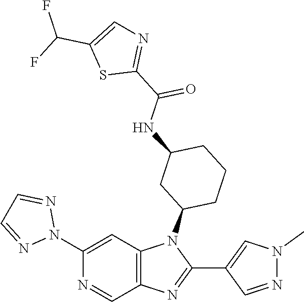 [SMILES: Cn1cc(-c2nc3cnc(-n4nccn4)cc3n2[C@@H]2CCC[C@H](NC(=O)c3ncc(C(F)F)s3)C2)cn1] | (400 MHZ, DMSO-d₆) δ 9.17 (d, 1 H), 8.87 (s, 1 H), 8.42 (s, 1 H), 8.32 (s, 1 H), 8.24 (s, 1 H), 8.18 (s, 2 H), 8.02 (s, 1 H), 7.62-7.28 (m, 1 H), 4.78- 4.70 (m, 1 H), 4.17 (br s, 1 H), 4.00 (s, 3 H), 2.67 (s, 1 H), 2.12 (br d, 2 H), 2.00 (br d, 1 H), 1.91 (br s, 2 H), 1.64 (br d, 2 H) | m/z 525.3 [M + H]⁺ |
| 46 | 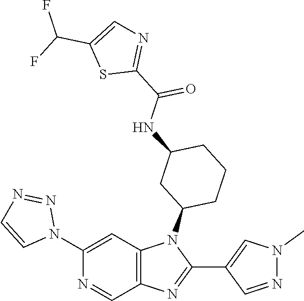 [SMILES: Cn1cc(-c2nc3cnc(-n4ccnn4)cc3n2[C@@H]2CCC[C@H](NC(=O)c3ncc(C(F)F)s3)C2)cn1] | (400 MHZ, CD₃OD) δ 8.84 (d, 2 H), 8.49 (s, 1 H), 8.28 (s, 1 H), 8.14 (s, 1 H), 8.03 (s, 1 H), 7.95 (s, 1 H), 7.32-7.01 (m, 1 H), 4.81-4.77 (m, 1 H), 4.24-4.12 (m, 1 H), 4.08 (s, 3 H), 2.58 (q, 1 H), 2.37 (br s, 2 H), 2.10 (br d, 3 H), 1.76- 1.65 (m, 2 H) | m/z 525.3 [M + H]⁺ |
| 47 | 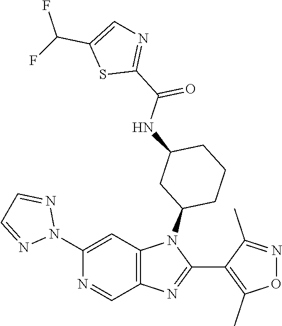 [SMILES: Cc1noc(C)c1-c1nc2cnc(-n3nccn3)cc2n1[C@@H]1CCC[C@H](NC(=O)c2ncc(C(F)F)s2)C1] | (400 MHZ, DMSO-d₆) δ 9.13 (d , 1 H), 9.00 (s, 1 H), 8.32 (d, 2 H), 8.20 (s, 2 H), 7.61- 7.29 (m, 1 H), 4.39 (brt, 1 H), 4.14 (br s, 1 H), 2.54-2.51 (m, 1 H), 2.48 (s, 3 H), 2.23 (s, 3 H), 2.07 (br d, 2 H), 1.98-1.76 (m, 3 H), 1.60 (br s, 2 H) | m/z 540.5 [M + H]⁺ |
| 48 | 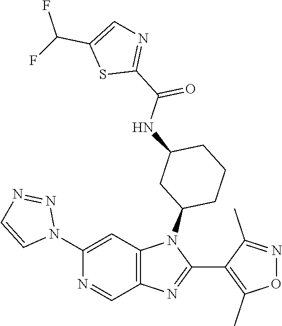 [SMILES: Cc1noc(C)c1-c1nc2cnc(-n3ccnn3)cc2n1[C@@H]1CCC[C@H](NC(=O)c2ncc(C(F)F)s2)C1] | (400 MHZ, DMSO-d₆) δ 9.14 (d, 1 H), 9.04 (s, 1 H), 8.91 (s, 1 H), 8.46 (s, 1 H), 8.31 (s, 1 H), 8.03 (s, 1 H), 7.62-7.28 (m, 1 H), 4.41 (br t, 1 H), 4.15 (br s, 1 H), 2.64-2.52 (m, 1 H), 2.49 (br s, 3 H), 2.24 (s, 3 H), 2.09 (br d , 2 H), 2.00- 1.79 (m, 3 H), 1.62 (br t, 2 H) | m/z 540.4 [M + H]⁺ |
| 51 | 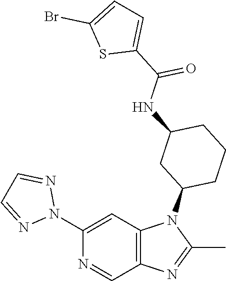 [SMILES: Cc1nc2cnc(-n3nccn3)cc2n1[C@@H]1CCC[C@H](NC(=O)c2ccc(Br)s2)C1] | (400 MHZ, CD₃OD) δ 8.74 (s, 1 H), 8.34 (s, 1 H), 8.04 (s, 2 H), 7.49 (d, 1 H), 7.12 (d, 1 H), 4.63 (br d, 1 H), 4.16 (br t, 1 H), 2.75 (s, 3 H), 2.43- 2.33 (m, 1 H), 2.31-2.18 (m, 2 H), 2.08 (br s, 3 H), 1.78- 1.67 (m, 1 H), 1.63-1.54 (m, 1 H) | m/z 488.2 [M + H]⁺ |
| 52 | 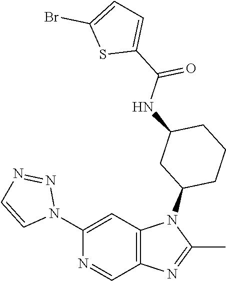 [SMILES: Cc1nc2cnc(-n3ccnn3)cc2n1[C@@H]1CCC[C@H](NC(=O)c2ccc(Br)s2)C1] | (400 MHZ, CD₃OD) δ 8.82 (s, 1 H), 8.77 (s, 1 H), 8.40 (s, 1 H), 7.93 (s, 1 H), 7.50 (d, 1 H), 7.13 (d, 1 H), 4.62 (br s, 1 H), 4.20-4.13 (m, 1 H), 2.76 (s, 3 H), 2.42-2.34 (m, 1 H), 2.32-2.22 (m, 2 H), 2.12-2.05 (m, 3 H), 1.73 (br d, 1 H), 1.64-1.57 (m, 1 H) | m/z 488.1 [M + H]⁺ |
| 72 | 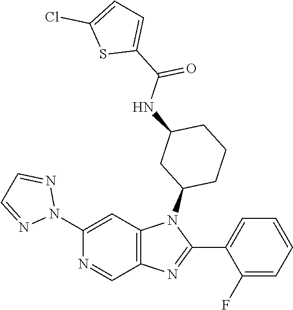 [SMILES: O=C(N[C@H]1CCC[C@@H](n2c(-c3ccccc3F)nc3cnc(-n4nccn4)cc32)C1)c1ccc(Cl)s1] | (400 MHZ, CDCl₃) δ 9.07 (s, 1 H), 8.20 (s, 1 H), 7.98 (s, 2 H), 7.70 -7.63 (m, 2 H), 7.44- 7.40 (m, 1 H), 7.34-7.32 (m, 1 H), 6.88 (d, 1 H), 6.17 (s, 1 H), 4.26 (s, 1 H), 4.09-4.07 (m, 1 H), 2.34-2.05 (m, 6 H), 1.49-1.42 (m, 2 H) | m/z 522.2 [M + H]⁺ |
| 76 | 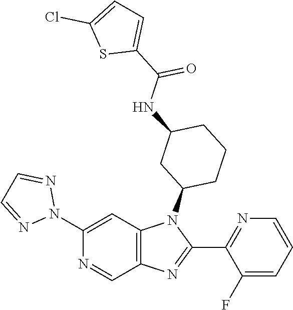 [SMILES: O=C(N[C@H]1CCC[C@@H](n2c(-c3ncccc3F)nc3cnc(-n4nccn4)cc32)C1)c1ccc(Cl)s1] | (400 MHZ, CD₃CN) & 8.67 (s, 1 H), 7.76-7.72 (m, 1 H), 7.60-7.54 (m, 1 H), 7.30-7.28 (m, 5 H), 6.90 (s, 1 H), 6.04 (s, 1 H), 4.88 (s, 1 H), 4.11 (s, 1 H), 2.50 (s, 1 H), 2.27 -2.19 (m, 4 H), 2.10-2.07 (m, 1 H), 1.59-1.49 (m, 2 H) | m/z 523.2 [M + H]⁺ |
| 84 | 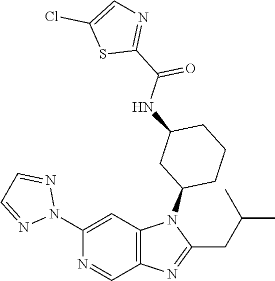 [SMILES: CC(C)Cc1nc2cnc(-n3nccn3)cc2n1[C@@H]1CCC[C@H](NC(=O)c2ncc(Cl)s2)C1] | (400 MHz, DMSO-d₆) δ 9.04 (br d, 1 H), 8.82 (s, 1 H), 8.18 (s, 1 H), 8.17 (s, 2 H), 8.06 (s, 1 H), 4.68 (br s, 1 H), 4.19 (br s, 1 H), 2.91 (d, 2 H), 2.36- 2.20 (m, 1 H), 2.15-1.53 (m, 9 H), 1.02 (d, 6 H) | m/z 485.2 [M + H]⁺ |
| 95 | 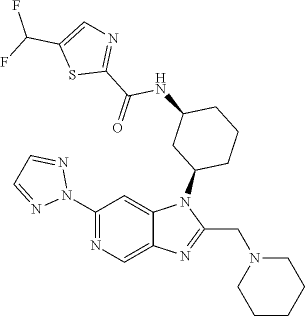 [SMILES: O=C(N[C@H]1CCC[C@@H](n2c(CN3CCCCC3)nc3cnc(-n4nccn4)cc32)C1)c1ncc(C(F)F)s1] | (400 MHZ, CD₃OD) δ 8.82 (s, 1 H), 8.40 (s, 1 H), 8.16 (s, 1 H), 8.07 (s, 1 H), 7.19 (t, 1 H), 5.01 (br s, 1 H), 4.19 (br s, 1 H), 3.91 (t, 2 H), 2.52- 2.49 (m, 5 H), 2.35-2.49 (m, 2 H), 2.16-2.13 (m, 3 H), 1.75-1.51 (m, δ H) | m/z 542.3 [M + H]⁺ |
| 99 | 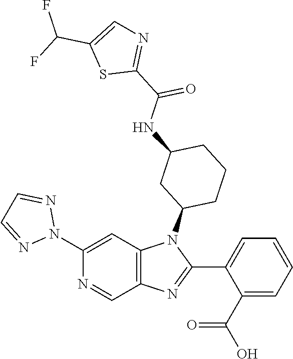 [SMILES: O=C(N[C@H]1CCC[C@@H](n2c(-c3ccccc3C(=O)O)nc3cnc(-n4nccn4)cc32)C1)c1ncc(C(F)F)s1] | (400 MHZ, CDCl₃) δ 9.05 (br s, 1 H), 8.17-8.15 (m, 2 H), 7.96-7.92 (m, 3H), 7.72-7.65 (m, 2 H), 7.54-7.48 (m, 2 H), 6.91 (t, 1 H), 4.11-4.06 (m, 1 H), 3.95-3.92 (m, 1 H), 2.64- 2.50 (m, 1 H), 2.10-2.09 (m, 1 H), 2.01-1.86 (m, 4 H), 1.55-1.35 (m, 2H) | m/z 565.3 [M + H]⁺ |
| 100 | 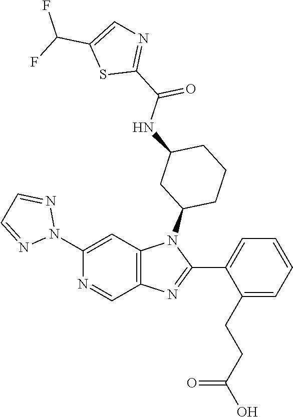 [SMILES: O=C(O)CCc1ccccc1-c1nc2cnc(-n3nccn3)cc2n1[C@@H]1CCC[C@H](NC(=O)c2ncc(C(F)F)s2)C1] | (400 MHZ, CDCl₃) δ 9.05 (br s, 1 H), 8.22 (br s, 1 H), 7.96- 7.93 (m, 3 H), 7.56 (br s, 2 H), 7.39 (br s, 2 H), 7.31-7.27 (m, 1 H), 6.89 (t, 1 H), 4.21- 4.15 (m, 1 H), 3.88 (br s, 1 H), 3.20-2.95 (m, 2 H), 2.88- 2.60 (m, 4 H), 2.33-2.16 (m, 2 H), 1.59-1.47 (br d, 4 H). | m/z 593.3 [M + H]⁺ |
| 131 | 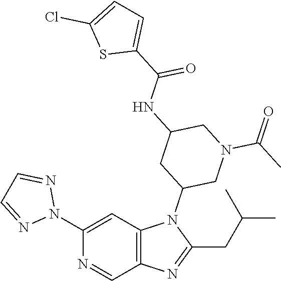 [SMILES: CC(=O)N1CC(NC(=O)c2ccc(Cl)s2)CC(n2c(CC(C)C)nc3cnc(-n4nccn4)cc32)C1] | (400 MHZ, CD₃OD) δ 9.02- 8.86 (m, 2 H), 8.61-8.51 (m, 1 H), 7.98-7.97 (m, 1 H), 7.60-7.57 (m, 1 H), 7.05-7.02 (m, 1 H), 4.79-4.71 (m, 2 H), 4.33-3.97 (m, 2 H), 3.51-3.45 (m, 0.5 H), 3.28-3.21 (m, 0.5 H), 3.07-3.01 (m, 1 H), 2.94- 2.71 (m, 2 H), 2.44-2.31 (m, 2 H), 2.29-2.16 (m, 3 H), 1.34-1.26 (m, 1 H), 1.12-1.06 (m, 6 H). | m/z 527.2 [M + H]⁺ |
| | (relative stereochemistry at centers 3 and 5 | | |
| | of piperidine ring is cis) | | |
| 132 | 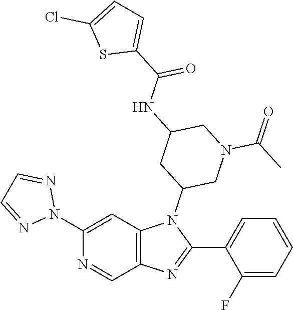 [SMILES: CC(=O)N1CC(NC(=O)c2ccc(Cl)s2)CC(n2c(-c3ccccc3F)nc3cnc(-n4nccn4)cc32)C1] | (400 MHZ, CD₃OD) δ 9.01 (s, 1 H), 8.92 (s, 1 H), 8.64 (s, 1 H), 8.00 (s, 1 H), 7.78-7.75 (m, 2 H), 7.58-7.45 (m, 3 H), 7.05-7.03 (m, 1 H), 4.81 (br s, 1 H), 4.36 (br s, 1 H), 4.28- 4.03 (m, 2 H), 3.52-3.45 (m, 1 H), 3.25-3.14 (m, 1 H), 2.84-2.70 (m, 1 H), 2.47-2.45 (m, 1 H), 2.19-2.09 (m, 3 H). | m/z 565.2 [M + H]⁺ |
| | (relative stereochemistry at centers 3 and 5 | | |
| | of piperidine ring is cis) | | |
| 140 | 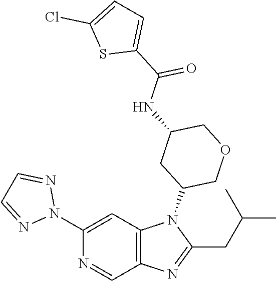 | (400 MHZ, CD₃OD) δ 8.81 (s, 1 H), 8.43 (s, 1 H), 8.08 (s, 2 H), 7.55 (d, 1 H), 7.03 (d, 1 H), 4.85-4.83 (m, 1 H), 4.47- 4.39 (m, 1 H), 4.19-4.12 (m, 1 H), 4.13-4.12 (m, 1 H), 4.11-4.06 (m, 1 H), 3.50 (t, 1 H), 3.00 (dd, 2 H), 2.73 (q, 1 H), 2.44 (br d, 1 H), 2.29 (dt, 1 H), 1.11 (dd, 6 H) | m/z 486.2 [M + H]⁺; (column: DAICEL CHIRALPAK AD(250 mm * 30 mm,10 um); mobile phase: [Neu-IPA]; B%: 35%-35%, min); Rt = 1.02 min 100% ee |
| 141 | 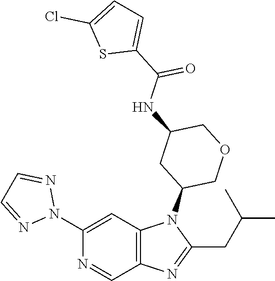 [SMILES: CC(C)Cc1nc2cnc(-n3nccn3)cc2n1[C@@H]1COC[C@H](NC(=O)c2ccc(Cl)s2)C1] | (400 MHZ, CD₃OD) δ 8.69 (s, 1 H), 8.31 (s, 1 H), 7.96 (s, 2 H), 7.43 (d, 1 H), 6.90 (d, 1 H), 4.71 (br s, 1 H), 4.34-4.26 (m, 1 H), 4.08-4.01 (m, 2 H), 3.99-3.93 (m, 1 H), 3.38 (t, 1 H), 2.88 (dd, 2 H), 2.61 (q, 1 H), 2.32 (br d, 1 H), 2.22- 2.12 (m, 1 H), 0.99 (dd, 6 H) | m/z 486.1 [M + H]⁺; (column: DAICEL CHIRALPAK AD(250 mm * 30mm, 10 um); mobile phase: [Neu-IPA]; B%: 35%-35%, min); Rt = 2.33 min 100% ee |
| 190 | 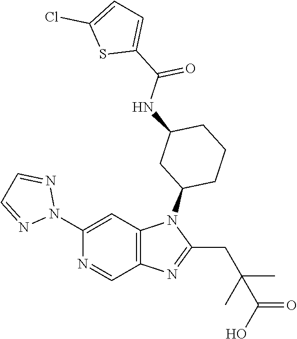 [SMILES: CC(C)(Cc1nc2cnc(-n3nccn3)cc2n1[C@@H]1CCC[C@H](NC(=O)c2ccc(Cl)s2)C1)C(=O)O] | (400 MHZ, CDCl₃) δ 8.02 (s, 1 H), 7.89 (s, 2 H), 7.41 (s, 1 H), 6.88-6.87 (m, 1 H), 6.09- 6.07 (m, 1 H), 4.66 -4.57 (m, 1 H), 4.06-4.04 (m, 1 H), 3.68-3.59 (m, 1 H), 2.98-2.80 (m, 2 H), 2.46-2.43 (m, 1 H), 2.10-2.03 (m, 2 H), 1.51-1.48 (m, 7 H), 1.27-1.18 (m, 4 H). | m/z 528.3 [M + H]⁺ |
| 300 | 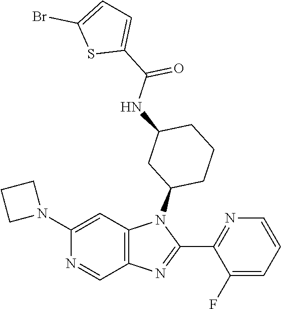 [SMILES: O=C(N[C@H]1CCC[C@@H](n2c(-c3ncccc3F)nc3cnc(N4CCC4)cc32)C1)c1ccc(Br)s1] | (400 MHZ, CD₃OD) δ 8.69-8.68 (m, 1 H), 8.52 (s, 1 H), 7.95-7.91 (m, 1 H), 7.78-7.73 (m, 1 H), 7.50-7.49 (m, 1 H), 7.14-7.12 (m, 2 H), 4.68-4.50 (m, 3 H), 3.87 (br s, 1 H), 3.59-3.57 (m, 2 H), 2.34-1.98 (m, δ H), 1.50- 1.47 (m, 2 H). | m/z 555.1 [M + H]⁺ |
| 301 | 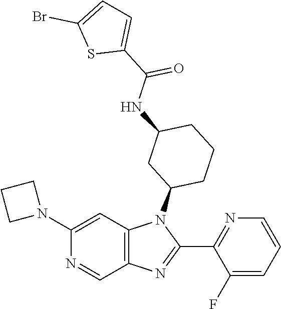 [SMILES: O=C(N[C@H]1CCC[C@@H](n2c(-c3ncccc3F)nc3cnc(N4CCC4)cc32)C1)c1ccc(Br)s1] | (400 MHZ, CD₃OD) δ 8.49 (s, 1 H), 7.77-7.72 (m, 1 H), 7.67- 7.65 (m, 1 H), 7.48-7.41 (m, 3 H), 7.14-7.10 (m, 2 H), 4.55- 4.47 (m, 2 H), 4.22-4.10 (m, 1 H), 3.82 (br s, 1 H), 3.59-3.56 (m, 2 H), 2.35-2.14 (m, 4 H), 2.13-2.04 (m, 1 H), 2.00 -1.97 (m, 3 H), 1.50-1.46 (m, 2 H). | m/z 554.1 [M + H]⁺ |

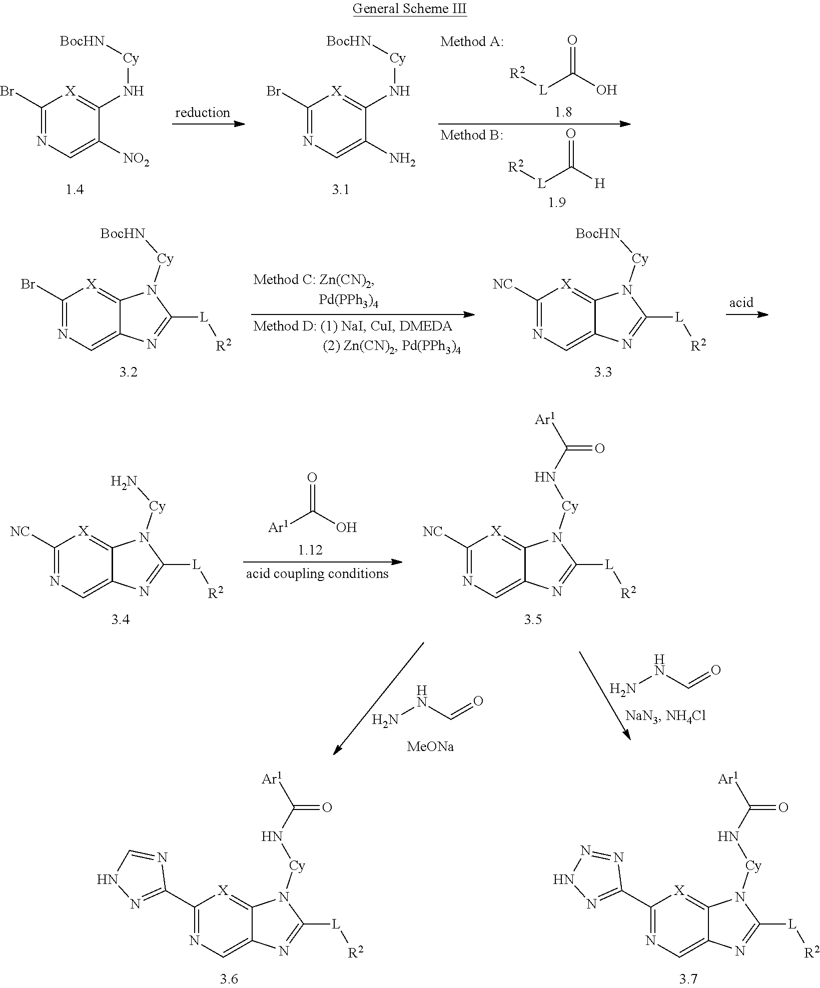

Compounds of formula 3.6 and 3.7 were obtained through the scheme depicted as General Scheme III. The nitro group of intermediate of formula 1.4 (prepared via General Scheme I) was reduced with a reducing agent such as iron in the presence of ammonium chloride to afford 2-bromopyridine-diamine compounds of formula 3.1. Subsequently, various L and R² groups were installed by coupling the pyridine-diamine compound of formula 3.1 with acid 1.8 to form the amide intermediate (Method A), which was subsequently cyclized under either basic or acidic conditions to form compounds of formula 3.2. Alternatively, the pyridine-diamine compound of formula 3.1 was condensed with aldehyde 1.9 (Method B), followed by cyclization and in situ oxidation to form compounds of formula 3.2. The bromide in the pyridine in compounds of formula 3.2 was converted to a cyano group either directly (Method C) or by first converting the bromide to an iodide, followed by cyanation (Method D) to afford compounds of formula 3.3. The Boc protecting group of compounds of formula 3.3 was removed under acidic conditions to afford compounds of formula 3.4. Ar¹ groups were then installed by reacting an Ar¹ carboxylic acid of formula 1.12 or a basic salt (i.e., Li, K, or Na) thereof with the amine of formula 3.4 using acid coupling conditions known in the art, such as using one of the following reagents—HATU, EDCI, HOBt—along with a base, such as DIPEA (Hunig's base) or TEA to afford compounds of formula 3.5. The cyano group of compounds of formula 3.5 was converted to a 1,2,4-triazole or tetrazole using formylhydrazine and either sodium methanolate or sodium azide, affording compounds of formulas 3.6 and 3.7.

### Preparation of Example 68 Via General Scheme III (Methods B and D)

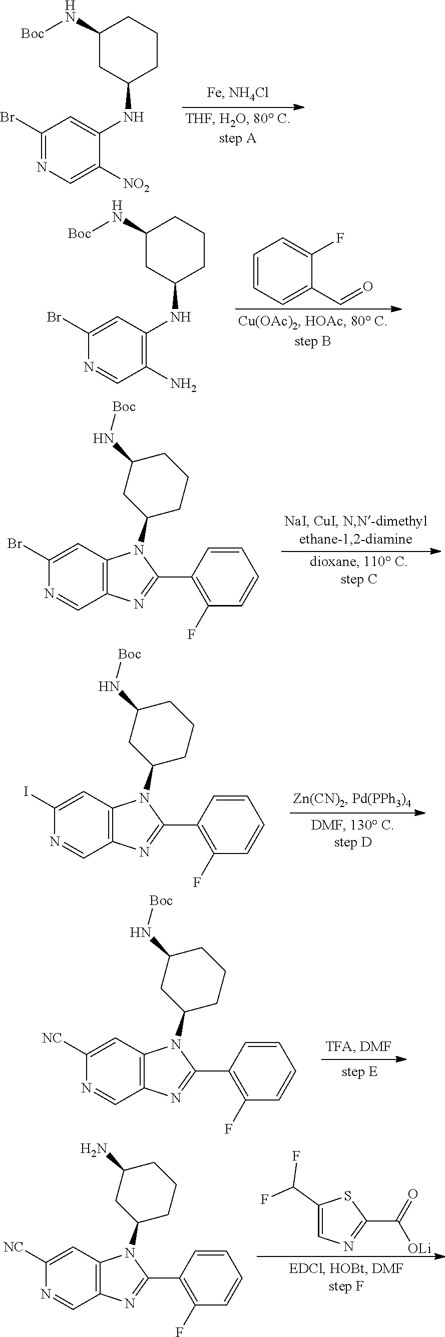

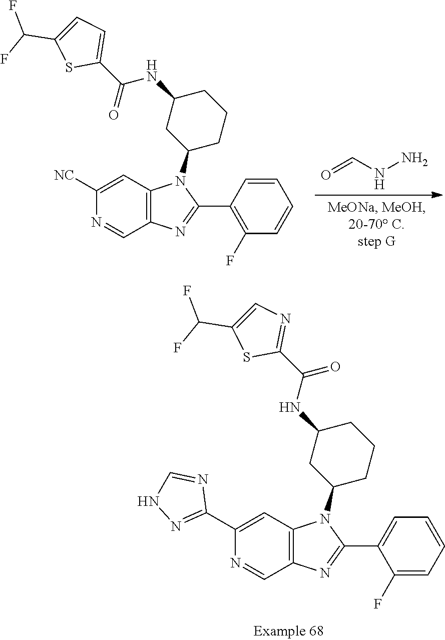

### Step A. Synthesis of tert-butyl ((1S,3R)-3-((5-amino-2-bromopyridin-4-yl)amino)cyclohexyl)carbamate

To a mixture of tert-butyl ((1S,3R)-3-((2-bromo-5-nitropyridin-4-yl)amino)cyclohexyl)carbamate (5 g, 12 mmol) in THF (40 mL) and H₂O (20 mL), were added NH₄CI (773 mg, 14.5 mmol) and Fe (3.36 g, 60.2 mmol). The reaction mixture was stirred at 80° C. for 4 hrs, and then allowed to cool. The reaction mixture was filtered, and the filtrate was concentrated to dryness to give tert-butyl ((1S,3R)-3-((5-amino-2-bromopyridin-4-yl)amino)cyclohexyl)carbamate (4.8 g), which was used without further purification. LC-MS: m/z 386.9 [M+H]⁺

### Step B. Synthesis of tert-butyl ((1S,3R)-3-(6-bromo-2-(2-fluorophenyl)-1H-imidazo[4,5-c]pyridin-1-yl)cyclohexyl)carbamate

To a mixture of tert-butyl ((1S,3R)-3-((5-amino-2-bromopyridin-4-yl)amino)cyclohexyl)carbamate (2 g, 5 mmol) and 2-fluorobenzaldehyde (644 mg, 5.19 mmol) in AcOH (20 mL) was added Cu(OAc)₂·H₂O (1.04 g, 5.19 mmol). The reaction mixture was stirred at 80° C. for 2 hrs, concentrated to dryness, and purified by silica gel column chromatography to give tert-butyl ((1S,3R)-3-(6-bromo-2-(2-fluorophenyl)-1H-imidazo[4,5-c]pyridin-1-yl)cyclohexyl)carbamate (1.5 g, 58.6% yield). LC-MS: m/z 489.1 [M+H]⁺.

### Step C. Synthesis of tert-butyl ((1S,3R)-3-(2-(2-fluorophenyl)-6-iodo-1H-imidazo[4,5-c]pyridin-1-yl)cyclohexyl)carbamate

To a solution of tert-butyl ((1S,3R)-3-(6-bromo-2-(2-fluorophenyl)-1H-imidazo[4,5-c]pyridin-1-yl)cyclohexyl)carbamate (1.5 g, 3.1 mmol) in dioxane (20 mL) were added NaI (919 mg, 6.13 mmol), CuI (175 mg, 920 μmol) and N,N′-dimethylethane-1,2-diamine (162 mg, 1.84 mmol). The reaction mixture was stirred at 110° C. for 60 hrs, then allowed to cool. The reaction mixture was washed with H₂O (30 mL) and extracted with DCM (25 mL×2). The combined organic layers were dried over Na₂SO₄, filtered, and concentrated to dryness to give tert-butyl ((1S,3R)-3-(2-(2-fluorophenyl)-6-iodo-1H-imidazo[4,5-c]pyridine-1-yl)cyclohexyl)carbamate (1.6 g), which was used without further purification. LC-MS: m/z 537.1 [M+H]⁺

### Step D. Synthesis of tert-butyl ((1S,3R)-3-(6-cyano-2-(2-fluorophenyl)-1H-imidazo[4,5-c]pyridine-1-yl)cyclohexyl)carbamate

To a mixture of tert-butyl ((1S,3R)-3-(2-(2-fluorophenyl)-6-iodo-1H-imidazo[4,5-c]pyridine-1-yl)cyclohexyl)carbamate (200 mg, 373 μmol) and Zn(CN)₂ (131 mg, 1.12 mmol) in DMF (2 mL) was added Pd(PPh₃)₄ (43.1 mg, 37.3 μmol). The reaction mixture was stirred at 130° C. for 12 hrs under N₂, then allowed to cool. DCM (10 mL) was added into the cooled reaction mixture at rt, and the organic portion washed with water (10 mL) and brine (15 mL). The organic layer was dried with Na₂SO₄, filtered, and concentrated to dryness. The residue was purified by silica gel column chromatography to give tert-butyl ((1S,3R)-3-(6-cyano-2-(2-fluorophenyl)-1H-imidazo[4,5-c]pyridin-1-yl)cyclohexyl)carbamate (35 mg, 21.6% yield). LC-MS: m/z 436.2 [M+H]⁺.

### Step E. Synthesis of 1-((1R,3S)-3-aminocyclohexyl)-2-(2-fluorophenyl)-1H-imidazo[4,5-c]pyridine-6-carbonitrile

To a mixture of tert-butyl ((1S,3R)-3-(6-cyano-2-(2-fluorophenyl)-1H-imidazo[4,5-c]pyridin-1-yl)cyclohexyl)carbamate (330 mg, 758 μmol) in DCM (5 mL) was added TFA (2.5 mL). The reaction mixture was stirred at rt for 1 hr, then concentrated to dryness to give 1-((1R,3S)-3-aminocyclohexyl)-2-(2-fluorophenyl)-1H-imidazo[4,5-c]pyridine-6-carbonitrile (340 mg, TFA salt), which was used without further purification. LC-MS: m/z 336.1 [M+H]⁺

### Step F. Synthesis of N-((1S,3R)-3-(6-cyano-2-(2-fluorophenyl)-1H-imidazo[4,5-c]pyridin-1-yl)cyclohexyl)-5-(difluoromethyl)thiophene-2-carboxamide

To a solution of 1-[(1R,3S)-3-aminocyclohexyl]-2-(2-fluorophenyl)imidazo[4,5-c]pyridine-6-carbonitrile (340 mg, 756.56 μmol, TFA salt) and [5-(difluoromethyl)thiazole-2-carbonyl]oxylithium (154 mg, 832 μmol) in DMF (5 mL) were added DIEA (489 mg, 3.78 mmol) and HATU (432 mg, 1.13 mmol). The reaction mixture was stirred at rt for 12 hrs, and then concentrated to dryness. The residue was purified by column chromatography (silica gel, PE/EtOAc=5/1 to 2/1) to give N-((1S,3R)-3-(6-cyano-2-(2-fluorophenyl)-1H-imidazo[4,5-c]pyridin-1-yl)cyclohexyl)-5-(difluoromethyl)thiophene-2-carboxamide (360 mg, 81.1% yield). LC-MS: m/z 497.1 [M+H]⁺

### Step G. Synthesis of 5-(difluoromethyl)-N-[(1S,3R)-3-[2-(2-fluorophenyl)-6-(1H-1,2,4-triazol-3-yl)imidazo[4,5-c]pyridin-1-yl]cyclohexyl]thiazole-2-carboxamide (Example 68)

To a mixture of N-[(1S,3R)-3-[6-cyano-2-(2-fluorophenyl)imidazo[4,5-c]pyridin-1-yl]cyclohexyl]-5-(difluoromethyl)thiazole-2-carboxamide (150 mg, 302 μmol) in MeOH (3 mL) was added sodium methanolate (54.4 mg, 302 μmol). The reaction mixture was stirred at rt for 3 hrs. Then formylhydrazine (36.3 mg, 604 μmol) was added. The reaction mixture was stirred at 70° C. for 12 hrs, then concentrated to dryness, and the residue purified by prep-HPLC (column: Welch Xtimate C18 150*25 mm*5 um; mobile phase: [water(0.225% FA)-ACN]; B %: 32%-52%, 8 min) to give Example 68 (34 mg, 20.9% yield). ¹H NMR (400 MHz, CDCl₃) δ 9.27 (s, 1H), 8.57 (s, 1H), 8.29 (s, 1H), 8.00 (s, 1H), 7.74-7.64 (m, 2H), 7.47-7.32 (m, 3H), 7.14-6.74 (m, 1H), 4.32 (br s, 1H), 4.10 (br dd, 1H), 2.50-2.28 (m, 3H), 2.20-2.06 (m, 3H), 1.66-1.48 (m, 2H). LC-MS: m/z 539.2 [M+H]⁺.

### Preparation of Examples 135 and 136 Via General Scheme III (Methods B and D)

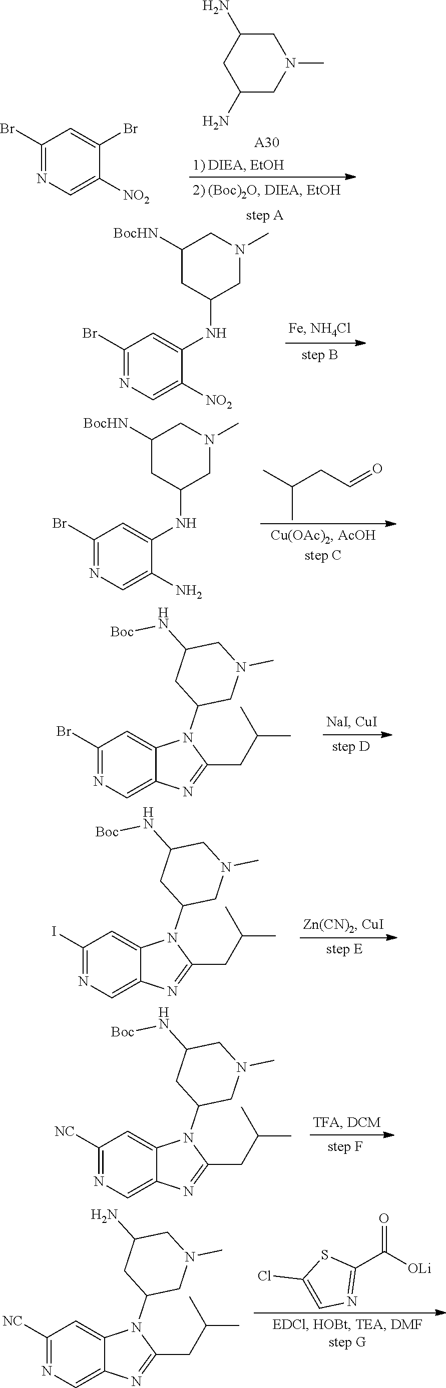

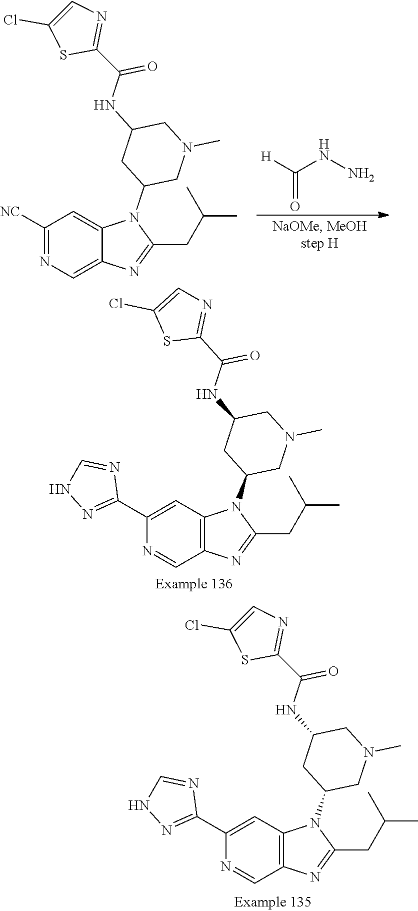

### Step A. Synthesis of tert-butyl (5-((2-bromo-5-nitropyridin-4-yl)amino)-1-methylpiperidin-3-yl)carbamate (relative stereochemistry at centers 3 and 5 of piperidine ring is cis)

A solution of 2,4-dibromo-5-nitro-pyridine (1.00 g, 3.56 mmol), 1-methylpiperidine-3,5-diamine (A30) (0.9 g, 4.5 mmol, 2HCl) and DIEA (2.88 g, 22.3 mmol, 3.88 mL) in EtOH (25 mL) was heated at 60° C. under nitrogen for 16 hrs. The reaction mixture was concentrated to dryness to give N³-(2-bromo-5-nitropyridin-4-yl)-1-methylpiperidine-3,5-diamine (2.1 g). LC-MS: m/z 332.0 [M+H]⁺

A solution of N³-(2-bromo-5-nitropyridin-4-yl)-1-methylpiperidine-3,5-diamine (2.1 g, 6.4 mmol), (Boc)₂O (2.08 g, 9.54 mmol, 2.19 mL) and DIEA (4.11 g, 31.8 mmol, 5.54 mL) in EtOH (10 mL) was stirred at rt under nitrogen for 16 hrs. The reaction mixture was concentrated to dryness. The residue was purified by column chromatography on silica gel to give tert-butyl (5-((2-bromo-5-nitropyridin-4-yl)amino)-1-methylpiperidin-3-yl)carbamate (2.9 g, 51.3% yield). LC-MS: m/z 432.1 [M+H]⁺

### Step B. Synthesis of tert-butyl (5-((5-amino-2-bromopyridin-4-yl)amino)-1-methylpiperidin-3-yl)carbamate (relative stereochemistry at centers 3 and 5 of piperidine ring is cis)

To a suspension of tert-butyl (5-((2-bromo-5-nitropyridin-4-yl)amino)-1-methylpiperidin-3-yl)carbamate (2.8 g, 6.5 mmol) and Fe (1.82 g, 32.5 mmol) in THF (30 mL) was added NH₄Cl (418 mg, 7.81 mmol) in H₂O (15 mL). After the addition was complete, the reaction mixture was stirred at 70° C. for 16 hrs, and then allowed to cool. The reaction mixture was filtered, and water (15 mL) was added. The aqueous portion was extracted with EtOAc (5×20 mL). The combined organic layers were washed with brine (20 mL), dried over Na₂SO₄, filtered, and concentrated to dryness. The residue was purified by column chromatography on silica gel to give tert-butyl (5-((5-amino-2-bromopyridin-4-yl)amino)-1-methylpiperidin-3-yl)carbamate (600 mg, 21.2% yield). LC-MS: m/z 400.2 [M+H]⁺

### Step C. Synthesis of tert-butyl (5-(6-bromo-2-isobutyl-1H-imidazo[4,5-c]pyridin-1-yl)-1-methylpiperidin-3-yl)carbamate (relative stereochemistry at centers 3 and 5 of piperidine ring is cis)

A mixture of tert-butyl (5-((5-amino-2-bromopyridin-4-yl)amino)-1-methylpiperidin-3-yl)carbamate (0.7 g, 1.75 mmol), Cu(OAc)₂·H₂O (349 mg, 1.75 mmol) and 3-methylbutanal (151 mg, 1.75 mmol) in AcOH (8 mL) was heated at 80° C. for 1 hr. The reaction mixture was concentrated to dryness, and water (10 mL) was added to the residue. The resulting mixture was adjusted to pH ˜8 by the addition of aq. ammonia solution. The aqueous portion was extracted with EtOAc (4×15 mL). The combined organic layers were washed with brine (15 mL), dried over Na₂SO₄, filtered, and concentrated to dryness to give a residue. The residue was purified by column chromatography on silica gel to give tert-butyl (5-(6-bromo-2-isobutyl-1H-imidazo[4,5-c]pyridin-1-yl)-1-methylpiperidin-3-yl)carbamate (0.42 g, 47.8% yield). LC-MS: m/z 466.3 [M+H]⁺

### Step D. Synthesis of tert-butyl (5-(6-iodo-2-isobutyl-1H-imidazo[4,5-c]pyridin-1-yl)-1-methylpiperidin-3-yl)carbamate (relative stereochemistry at centers 3 and 5 of piperidine ring is cis)

To a solution of tert-butyl (5-(6-bromo-2-isobutyl-1H-imidazo[4,5-c]pyridin-1-yl)-1-methylpiperidin-3-yl)carbamate (0.32 g, 686 μmol) in 1,4-dioxane (10 mL) were added NaI (206 mg, 1.37 mmol), CuI (39.2 mg, 206 μmol) and N,N′-dimethylethane-1,2-diamine (36.3 mg, 412 μmol, 44.3 μL). After the addition was complete, the reaction mixture was heated at 110° C. for 16 hrs, and then allowed to cool. Water (20 mL) was added, and the aqueous portion was extracted with EtOAc (3×25 mL). The combined organic layers were washed with brine (15 mL), dried over Na₂SO₄, filtered, and concentrated to dryness to give tert-butyl (5-(6-iodo-2-isobutyl-1H-imidazo[4,5-c]pyridine-1-yl)-1-methylpiperidin-3-yl)carbamate (0.45 g), which was used without further purification. LC-MS: m/z 514.3 [M+H]⁺

### Step E. Synthesis of tert-butyl (5-(6-cyano-2-isobutyl-1H-imidazo[4,5-c]pyridine-1-yl)-1-methylpiperidin-3-yl)carbamate (relative stereochemistry at centers 3 and 5 of piperidine ring is cis)

To a mixture of tert-butyl (5-(6-iodo-2-isobutyl-1H-imidazo[4,5-c]pyridine-1-yl)-1-methylpiperidin-3-yl)carbamate (0.35 g, 682 μmol) and Zn(CN)₂ (400 mg, 3.41 mmol) in DMF (5 mL) was added Pd(PPh₃)₄ (78.8 mg, 68.2 μmol) under nitrogen. After the addition was complete, the reaction mixture was heated at 130° C. under nitrogen for 16 hrs, before allowed to cool to rt. DCM (40 mL) was added, and the organic portion was washed with water (15 mL), brine (15 mL), filtered and concentrated to dryness. The residue was purified by column chromatography on silica gel to give tert-butyl (5-(6-cyano-2-isobutyl-1H-imidazo[4,5-c]pyridin-1-yl)-1-methylpiperidin-3-yl)carbamate (200 mg, 71.1% yield). LC-MS: m/z 413.4 [M+H]⁺

### Step F. Synthesis of 1-(5-amino-1-methylpiperidin-3-yl)-2-isobutyl-1H-imidazo[4,5-c]pyridine-6-carbonitrile (relative stereochemistry at centers 3 and 5 of piperidine ring is cis)

To a solution of tert-butyl (5-(6-cyano-2-isobutyl-1H-imidazo[4,5-c]pyridin-1-yl)-1-methylpiperidin-3-yl)carbamate (0.2 g, 485 μmol) in DCM (2 mL) was added TFA (4.22 g, 37.0 mmol, 2.74 mL). After the addition was completed, the reaction mixture was stirred at rt for 1 hr. The reaction mixture was concentrated to dryness to give 1-(5-amino-1-methylpiperidin-3-yl)-2-isobutyl-1H-imidazo[4,5-c]pyridine-6-carbonitrile (150 mg), which was used without further purification. LC-MS: m/z 313.2 [M+H]⁺

### Step G. Synthesis of 5-chloro-N-(5-(6-cyano-2-isobutyl-1H-imidazo[4,5-c]pyridin-1-yl)-1-methylpiperidin-3-yl)thiazole-2-carboxamide (relative stereochemistry at centers 3 and 5 of piperidine ring is cis)

To a solution of 1-(5-amino-1-methylpiperidin-3-yl)-2-isobutyl-1H-imidazo[4,5-c]pyridine-6-carbonitrile (0.15 g, 480 μmol), 5-chlorothiazole-2-carboxylic acid (81.9 mg, 480 μmol, lithium salt), DIEA (124 mg, 960 μmol, 168 μL) and HOBt (97.3 mg, 720 μmol) in DMF (5 mL) was added EDCI (138 mg, 720 μmol). After the addition was complete, the reaction mixture was stirred at rt for 16 hrs. The reaction mixture was filtered, and the filtrate was concentrated to dryness. The residue was purified by column chromatography on silica gel to give 5-chloro-N-(5-(6-cyano-2-isobutyl-1H-imidazo[4,5-c]pyridin-1-yl)-1-methylpiperidin-3-yl)thiazole-2-carboxamide (0.15 g), which was used without further purification. LC-MS: m/z 458.3 [M+H]⁺

### Step H. Synthesis of 5-chloro-N-((3S,5R)-5-(2-isobutyl-6-(1H-1,2,4-triazol-3-yl)-1H-imidazo[4,5-c]pyridin-1-yl)-1-methylpiperidin-3-yl)thiazole-2-carboxamide (Example 135) and 5-chloro-N-((3R,5S)-5-(2-isobutyl-6-(1H-1,2,4-triazol-3-yl)-1H-imidazo[4,5-c]pyridin-1-yl)-1-methylpiperidin-3-yl)thiazole-2-carboxamide (Example 136)

To a solution of 5-chloro-N-(5-(6-cyano-2-isobutyl-1H-imidazo[4,5-c]pyridin-1-yl)-1-methylpiperidin-3-yl)thiazole-2-carboxamide (150 mg, 327.5 μmol) in MeOH (4 mL) was added MeONa (59.0 mg, 328 μmol, 30% wt). After the addition was complete, the reaction mixture was stirred at rt for 3.5 hrs. Then formylhydrazine (39.3 mg, 655 μmol) was added, and the reaction mixture heated at 70° C. for another 16 hrs. The reaction mixture was concentrated to dryness. The residue was purified by column chromatography on silica gel and further purified by prep-HPLC to give 5-chloro-N-(5-(2-isobutyl-6-(1H-1,2,4-triazol-3-yl)-1H-imidazo[4,5-c]pyridin-1-yl)-1-methylpiperidin-3-yl)thiazole-2-carboxamide (25 mg, 14% yield). LC-MS: m/z 500.3 [M+H]⁺

The enantiomers of 5-chloro-N-(5-(2-isobutyl-6-(1H-1,2,4-triazol-3-yl)-1H-imidazo[4,5-c]pyridin-1-yl)-1-methylpiperidin-3-yl)thiazole-2-carboxamide (25 mg, 50 μmol) were separated by SFC (SFC: column: DAICEL CHIRALPAK IG (250 mm*30 mm, 10 um); mobile phase: [0.1% NH₃H₂O IPA]; B %: 40%-40%, min.) to give enantiomer 1: 5-chloro-N-((3S,5R)-5-(2-isobutyl-6-(1H-1,2,4-triazol-3-yl)-1H-imidazo[4,5-c]pyridin-1-yl)-1-methylpiperidin-3-yl)thiazole-2-carboxamide (Example 135) (6.5 mg, 25.5% yield, 100% ee, Rt=1.308 min) and enantiomer 2: 5-chloro-N-((3R,5S)-5-(2-isobutyl-6-(1H-1,2,4-triazol-3-yl)-1H-imidazo[4,5-c]pyridin-1-yl)-1-methylpiperidin-3-yl)thiazole-2-carboxamide (Example 136) (4.2 mg, 16.4% yield, 99.9% ee, Rt=1.529 min).
Example 135: ¹H NMR (400 MHz, CD₃OD) δ 8.97 (s, 1H), 8.55 (s, 2H), 7.84 (s, 1H), 4.83-4.77 (m, 1H), 4.47-4.39 (m, 1H), 3.22 (dd, 1H), 3.09-2.93 (m, 4H), 2.61-2.55 (m, 1H), 2.50 (s, 3H), 2.41-2.22 (m, 3H), 1.11 (t, 6H); LC-MS: m/z 500.2 [M+H]⁺.
Example 136: ¹H NMR (400 Hz, CD₃OD) δ 8.97 (s, 1H), 8.56 (s, 2H), 7.84 (s, 1H), 4.82-4.77 (m, 1H), 4.44-4.40 (N, 1H), 3.22 (dd, 1H), 3.09-2.94 (m, 4H), 2.61-2.55 (m, 1H), 2.50 (s, 3H), 2.41-2.23 (N, 3H), 1.12 (t, 6H); LC-MS: m/z 500.2 [M+H]⁺.

General Scheme III set forth above was used to synthesize the following compounds by using appropriate starting materials:

| Ex. No. | Structure | 1H NMR | LC-MS and % ee |
|---|---|---|---|
| 50 | 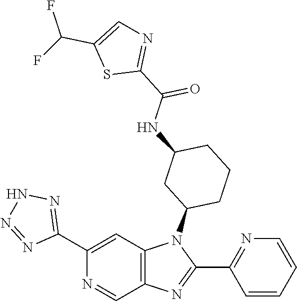 [SMILES: O=C(N[C@H]1CCC[C@@H](n2c(-c3ccccn3)nc3cnc(-c4nn[nH]n4)cc32)C1)c1ncc(C(F)F)s1] | (400 MHZ, CDCl₃) δ 9.34 (s, 1 H), 8.89-8.76 (m, 2 H), 8.37 (d, 1 H), 8.09- 7.94 (m, 2 H), 7.58-7.43 (m, 2 H), 7.10-6.74 (m, 1 H), 5.95 (br s, 1 H), 4.34- 4.10 (m, 1 H), 2.65-2.11 (m, 6 H), 1.76-1.60 (m, 2 H). | m/z 523.2 [M + H]⁺ |
| 53 | 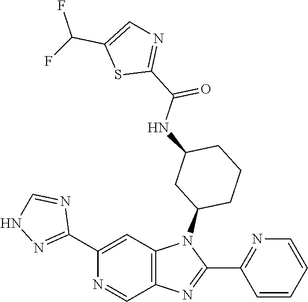 [SMILES: O=C(N[C@H]1CCC[C@@H](n2c(-c3ccccn3)nc3cnc(-c4nc[nH]n4)cc32)C1)c1ncc(C(F)F)s1] | (400 MHZ, CDCl₃) δ 9.20 (s, 1 H), 8.80 (d, 1 H), 8.50 (s, 1 H), 8.34 (d, 1 H), 8.17 (s, 1 H), 8.06- 7.91 (m, 2 H), 7.50 (dd, 1 H), 7.35 (br d, 1 H), 7.09- 6.77 (m, 1 H), 5.88 (br t, 1 H), 4.26-4.15 (m, 1 H), 2.57 (br d, 1 H), 2.45-2.20 (m, 4 H), 2.08-2.15 (m, 1 H), 1.55-1.73 (m, 2 H). | m/z 522.0 [M + H]⁺ |
| 75 | 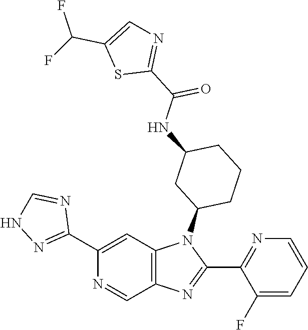 [SMILES: O=C(N[C@H]1CCC[C@@H](n2c(-c3ncccc3F)nc3cnc(-c4nc[nH]n4)cc32)C1)c1ncc(C(F)F)s1] | (400 MHZ, CD₃OD) δ 9.24 (s, 1 H), 8.98-8.74 (m, 3 H), 8.59 (br s, 1 H), 8.16 (t, 1 H), 8.03-7.93 (m, 1 H), 7.80 (dt, 1 H), 7.34-7.02 (m, 1 H), 4.99- 4.91 (m, 1 H), 4.15-4.01 (m, 1 H), 2.61-2.33 (m, 3 H), 2.22 (br d, 1 H), 2.13- 2.05 (m, 2 H), 1.78-1.54 (m, 2 H). | m/z 540.2 [M + H]⁺ |
| 80 | 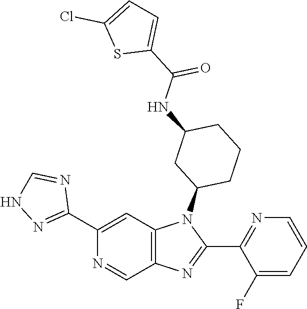 [SMILES: O=C(N[C@H]1CCC[C@@H](n2c(-c3ncccc3F)nc3cnc(-c4nc[nH]n4)cc32)C1)c1ccc(Cl)s1] | (400 MHZ, CDCl₃) δ 9.25 (s, 1 H), 8.65 (d, 1 H), 8.50 (s, 1 H), 8.23 (br s, 1 H), 7.75-7.55 (m, 3 H), 6.89 (br d, 2 H), 4.86-4.68 (m, 1 H), 3.97-4.15 (m, 1 H), 2.46-1.90 (m, 6 H), 1.52-1.19 (m, 2 H). | m/z 523.2 [M + H]⁺ |
| 82 | 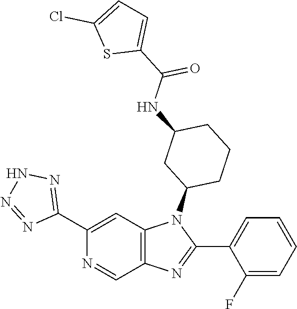 [SMILES: O=C(N[C@H]1CCC[C@@H](n2c(-c3ccccc3F)nc3cnc(-c4nn[nH]n4)cc32)C1)c1ccc(Cl)s1] | (400 MHZ, CD₃OD) δ 9.16 (s, 1 H), 8.74 (s, 1 H), 7.81-7.70 (m, 2 H), 7.57- 7.42 (m, 3 H), 7.01 (d, 1 H), 4.34 (br s, 1 H), 3.97 (br t, 1 H), 2.53-2.27 (m, 3 H), 2.14-2.01 (m, 3 H), 1.70-1.53 (m, 2 H). | m/z 523.2 [M + H]⁺ |
| 83 | 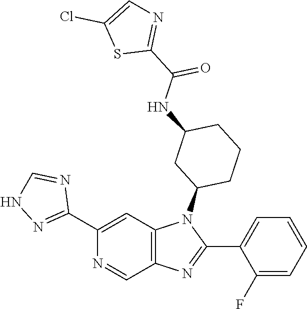 [SMILES: O=C(N[C@H]1CCC[C@@H](n2c(-c3ccccc3F)nc3cnc(-c4nc[nH]n4)cc32)C1)c1ncc(Cl)s1] | (400 MHZ, CDCl₃) δ 9.21 (s, 1 H), 8.55 (s, 1 H), 8.22 (s, 1 H), 7.72-7.63 (m, 3 H), 7.46-7.31 (m, 2 H), 7.26 (br d, 1 H), 4.29 (br s, 1 H), 4.07 (br d, 1 H), 2.46-2.29 (m, 3 H), 2.17- 2.04 (m, 3 H), 1.63-1.49 (m, 2 H). | m/z 523.2 [M + H]⁺ |
| 85 | 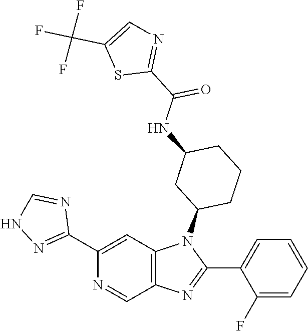 [SMILES: O=C(N[C@H]1CCC[C@@H](n2c(-c3ccccc3F)nc3cnc(-c4nc[nH]n4)cc32)C1)c1ncc(C(F)(F)F)s1] | (400 MHZ, CDCl₃) δ 9.21 (s, 1 H), 8.56 (s, 1 H), 8.24-8.12 (m, 2 H), 7.74- 7.63 (m, 2 H), 7.48-7.31 (m, 3 H), 4.36-4.23 (m, 1 H), 4.10 (br d, 1 H), 2.47- 2.30 (m, 3 H), 2.20-2.06 (m, 3 H), 1.67-1.49 (m, 2 H). | m/z 557.2 [M + H]⁺ |
| 115 | 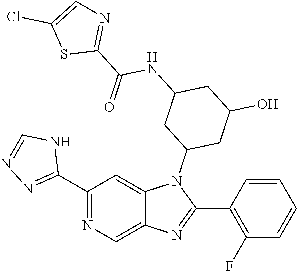 [SMILES: O=C(NC1CC(O)CC(n2c(-c3ccccc3F)nc3cnc(-c4nnc[nH]4)cc32)C1)c1ncc(Cl)s1] | (400 MHZ, CD₃OD) δ 9.11 (s, 1 H), 8.65 (s, 1 H), 7.82 (s, 1 H), 7.72-7.67 (m, 2 H), 7.51-7.47 (m, 2 H), 4.78 (br s, 1 H), 4.45- 4.35 (m, 2 H), 2.58-2.52 (m, 2 H), 2.30-2.26 (m, 1 H), 2.14-2.10 (m, 2 H), 1.98-1.95 (m, 1 H). | m/z 539.2 [M + H]⁺ |
| | (relative stereochemistry at centers 1 and 3 of | | |
| | cyclohexyl ring is cis with —OH at center 5 trans) | | |
| 133 | 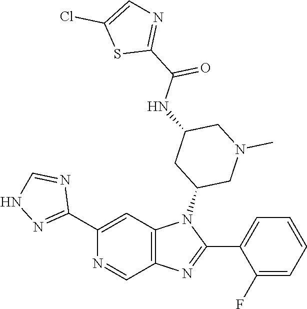 [SMILES: CN1C[C@@H](NC(=O)c2ncc(Cl)s2)C[C@@H](n2c(-c3ccccc3F)nc3cnc(-c4nc[nH]n4)cc32)C1] | (400 MHZ, CDCl₃) δ 9.19 (s, 1 H), 8.55 (s, 1 H), 8.17 (s, 1 H), 7.72-7.61 (m, 3 H), 7.44-7.29 (m, 3 H), 4.63-4.49 (m, 1 H), 4.34 (br dd, 1 H), 3.20 (br dd, 1 H), 3.11-3.02 (m, 1 H), 2.89 (t, 1 H), 2.53-2.36 (m, 5 H), 2.22 (t, 1 H). | m/z 538.2 [M + H]⁺; (column: DAICEL CHIRALPAK IG (250 mm * 30 mm, 10 um); mobile phase: [0.1% NH₃H₂O IPA]; B%: 35%-35%, min); Rt = 1.440 min 100% ee |
| 134 | 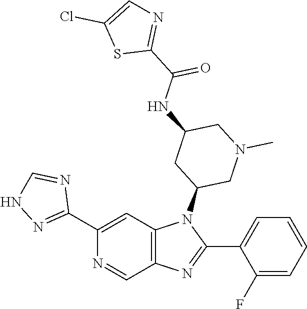 [SMILES: CN1C[C@H](NC(=O)c2ncc(Cl)s2)C[C@H](n2c(-c3ccccc3F)nc3cnc(-c4nc[nH]n4)cc32)C1] | (400 MHZ, CDCl₃) δ 9.18 (s, 1 H), 8.56 (s, 1 H), 8.17 (s, 1 H), 7.71-7.60 (m, 3 H), 7.44-7.29 (m, 3 H), 4.63-4.50 (m, 1 H), 4.41- 4.26 (m, 1 H), 3.20 (br dd, 1 H), 3.11-3.02 (m, 1 H), 2.88 (t, 1 H), 2.51-2.39 (m, 5 H), 2.27-2.19 (m, 1 H). | m/z 538.1 [M + H]⁺; (column: DAICEL CHIRALPAK IG (250 mm * 30 mm, 10 um); mobile phase: [0.1% NH₃H₂O IPA]; B%: 35%-35%, min); Rt = 2.092 min 97.5% ee |

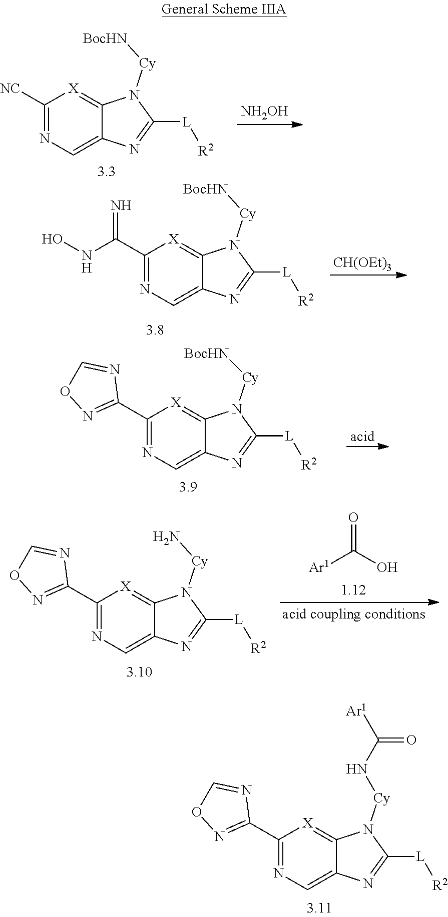

Compounds of formula 3.11 were obtained through the scheme depicted as General Scheme IIIA. The cyano moiety of a compound of formula 3.3 (prepared in accordance with General Scheme III) was converted to a 1,2,4-oxadiazole using hydroxylamine hydrochloride followed by triethyl orthoformate to afford compounds of formula 3.9. The Boc protecting group of compounds of formula 3.9 was removed under acidic conditions to afford compounds of formula 3.10. Ar¹ groups were then installed by reacting an Ar¹ carboxylic acid of formula 1.12 or a basic salt (i.e., Li, K, or Na) thereof with the amine of formula 3.10 using acid coupling conditions known in the art, such as using one of the following reagents—HATU, EDCI, HOBt—along with a base, such as DIPEA (Hunig's base) or TEA to afford compounds of formula 3.11.

### Preparation of Example 321 Via General Scheme IIIA

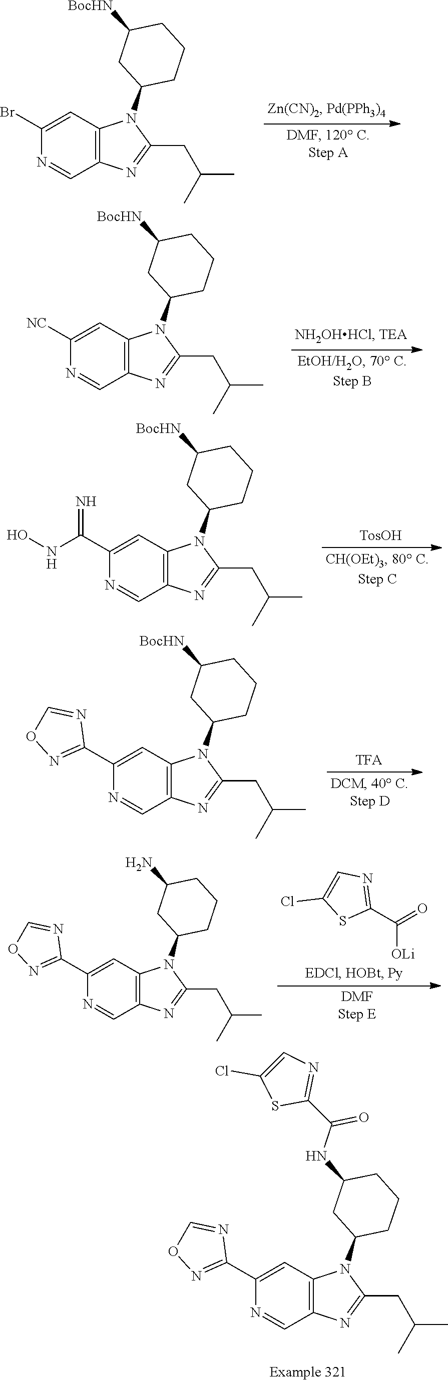

### Step A. Synthesis of tert-butyl ((1S,3R)-3-(6-cyano-2-isobutyl-1H-imidazo[4,5-c]pyridin-1-yl)cyclohexyl)carbamate

A mixture of tert-butyl ((1S,3R)-3-(6-bromo-2-isobutyl-1H-imidazo[4,5-c]pyridin-1-yl)cyclohexyl)carbamate (400 mg, 886 μmol), zinc cyanide (1.04 g, 8.86 mmol), Pd(PPh₃)₄ (256 mg, 222 μmol) in DMF (10 mL) was stirred at 120° C. for 3 hrs under N₂ atmosphere. Water (20 mL) was added to the reaction mixture, and the aqueous portion extracted with EtOAc (20 mL×3). The combined organic layers were washed with brine (10 mL), dried over Na₂SO₄, filtered, and concentrated to dryness. The residue was purified by flash column chromatography on silica gel to afford tert-butyl ((1S,3R)-3-(6-cyano-2-isobutyl-1H-imidazo[4,5-c]pyridin-1-yl)cyclohexyl)carbamate (253 mg, 72% yield). LC-MS: m/z 398 [M+H]⁺.

### Step B. Synthesis of tert-butyl ((1S,3R)-3-(6-(N-hydroxycarbamimidoyl)-2-isobutyl-1H-imidazo[4,5-c]pyridin-1-yl)cyclohexyl)carbamate

To a solution of tert-butyl ((1S,3R)-3-(6-cyano-2-isobutyl-1H-imidazo[4,5-c]pyridin-1-yl)cyclohexyl)carbamate (250 mg, 629 μmol) in EtOH (10 mL) and water (1 mL) were added Et₃N (318 mg, 3.14 mmol) and hydroxylamine hydrochloride (131 mg, 1.89 mmol). The reaction mixture was stirred at 70° C. for 5 hrs and then concentrated to dryness. The residue was purified by C18 column chromatography to give tert-butyl ((1S,3R)-3-(6-(N-hydroxycarbamimidoyl)-2-isobutyl-1H-imidazo[4,5-c]pyridin-1-yl)cyclohexyl)carbamate (180 mg, 67% yield). LC-MS: m/z 431 [M+H]⁺.

### Step C. Synthesis of tert-butyl ((1S,3R)-3-(2-isobutyl-6-(1,2,4-oxadiazol-3-yl)-1H-imidazo[4,5-c]pyridin-1-yl)cyclohexyl)carbamate

To a solution of tert-butyl ((1S,3R)-3-(6-(N-hydroxycarbamimidoyl)-2-isobutyl-1H-imidazo[4,5-c]pyridin-1-yl)cyclohexyl)carbamate (200 mg, 465 μmol) in triethyl orthoformate (1 mL) was added TsOH (88 mg, 465 μmol). The reaction mixture was stirred at 80° C. for 2 hrs. Water (10 mL) was added, and the aqueous portion extracted with EtOAc (10 mL×3). The organic layers were combined and washed with brine (10 mL), dried over Na₂SO₄, filtered, and concentrated to dryness. The residue was purified by flash column chromatography on silica gel to afford tert-butyl ((1S,3R)-3-(2-isobutyl-6-(1,2,4-oxadiazol-3-yl)-1H-imidazo[4,5-c]pyridin-1-yl)cyclohexyl)carbamate (130 mg, 64% yield). LC-MS: m/z 441 [M+H]⁺.

### Step D. Synthesis of (1S,3R)-3-(2-isobutyl-6-(1,2,4-oxadiazol-3-yl)-1H-imidazo[4,5-c]pyridin-1-yl)cyclohexan-1-amine

To a solution of tert-butyl ((1S,3R)-3-(2-isobutyl-6-(1,2,4-oxadiazol-3-yl)-1H-imidazo[4,5-c]pyridin-1-yl)cyclohexyl)carbamate (110 mg, 250 μmol) in DCM (2.5 mL) was added TFA (0.5 mL). The reaction mixture was stirred at 40° C. for 2 hrs and then concentrated to dryness to afford (1S,3R)-3-(2-isobutyl-6-(1,2,4-oxadiazol-3-yl)-1H-imidazo[4,5-c]pyridin-1-yl)cyclohexan-1-amine (100 mg), which was used without further purification. LC-MS: m/z 341 [M+H]⁺.

### Step E. Synthesis of 5-chloro-N-((1S,3R)-3-(2-isobutyl-6-(1,2,4-oxadiazol-3-yl)-1H-imidazo[4,5-c]pyridin-1-yl)cyclohexyl)thiazole-2-carboxamide (Example 321)

To a solution of (1S,3R)-3-(2-isobutyl-6-(1,2,4-oxadiazol-3-yl)-1H-imidazo[4,5-c]pyridin-1-yl)cyclohexan-1-amine (130 mg, 382 μmol) and lithium 5-chlorothiazole-2-carboxylate (97 mg, 573 μmol) in DMF (2 mL) were added EDCI (146 mg, 764 μmol), HOBt (117 mg, 764 μmol) and pyridine (242 mg, 3.05 mmol). The reaction mixture was stirred at rt for 16 hrs. Water (10 mL) was added, and the aqueous portion extracted with EtOAc (10 mL×3). The combined organic layers were washed with brine (10 mL), dried over Na₂SO₄, filtered, and concentrated to dryness. The residue was purified by prep-HPLC to give Example 321 (32 mg, 17% yield). ¹H NMR (400 MHz, DMSO-d₆) δ 9.77 (s, 1H), 9.07 (d, 1H), 9.01 (s, 1H), 8.38 (s, 1H), 8.05 (s, 1H), 4.75-4.65 (m, 1H), 4.28-4.10 (m, 1H), 2.92 (d, 2H), 2.62-2.54 (m, 1H), 2.29-2.17 (m, 1H), 2.09-1.86 (m, 5H), 1.71-1.57 (m, 2H), 1.01 (dd, 6H); LC-MS: m/z 486.2 [M+H]⁺.

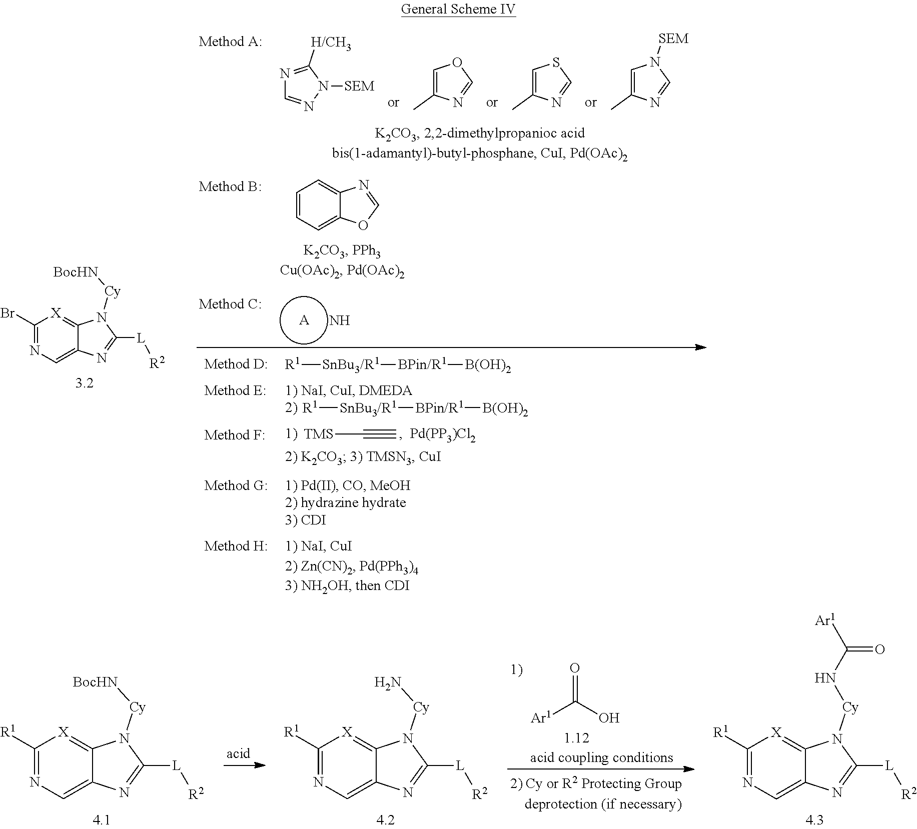

Compounds of formula 4.3 were obtained through the scheme depicted as General Scheme IV. R¹ groups were installed by reacting the bromide in compounds of formula 3.2 with the reagents specified in Methods A, B, C, D, E, F, G, and H to afford compounds of formula 4.1. Under Methods A and B, R¹ heteroaryls such as 1,2,4-triazole, 4-methyloxazole, 4-methylthiazole, imidazole, and benzoxazole were installed via a C—H functionalization reaction of the heteroaryl. Under Method C, the bromide of formula 3.2 was displaced by the NH of heterocyclic amines (saturated or aromatic) in the presence of a suitable base such as cesium carbonate and/or metal catalysts such as CuI. Under Method D, the bromide of formula 3.2 was converted to a R¹ heteroaryl or aryl using a Stille coupling (R¹-SnBu₃) or Suzuki coupling (R¹-BPin or R¹—B(OH)₂) to afford compounds of formula 4.1. In some cases, the bromide moiety needed to be converted to the more reactive iodide moiety using the reaction conditions in Method E prior to the Stille or Suzuki coupling. Under Method F, a 1,2,3-triazole was installed by (1) converting the bromide of formula 3.2 to an ethynyltrimethylsilane moiety, (2) removing the trimethylsilane group under basic conditions such as potassium carbonate, and (3) reacting the intermediate with TMSN₃ in the presence of a metal catalyst such as CuI. Under Method G, an 1,3,4-oxadiazol-2-one was installed by converting the bromide moiety to a methyl ester, followed by reacting the methyl ester with hydrazine hydrate and cyclizing with CDI. Under Method H, an 1,2,4-oxadiazol-one was installed by converting the bromide moiety of formula 4.1 to an iodide moiety, which was then subsequently converted to a cyano moiety via reaction with Zn(CN)₂ and Pd(PPh₃)₄. The cyano moiety was then reacted hydroxylamine and cyclized with CDI. The Boc protecting group in compounds of formula 4.1 was removed under acidic conditions to afford Compound 4.2. Ar¹ groups were then installed by reacting an Ar¹ carboxylic acid of formula 1.12 or a basic salt (i.e., Li, K, or Na) thereof with the amine of formula 4.2 using acid coupling conditions known in the art, such as using one of the following reagents—HATU, EDCI, HOBt—along with a base, such as DIPEA (Hunig's base) or TEA to afford compounds of formula 4.3. If compounds of formula 4.3 still contain any protecting groups on the Cy or R² groups, they may be removed under acidic conditions such as hydrochloric acid.

### Preparation of Example 5 Via General Scheme IV (Method D)

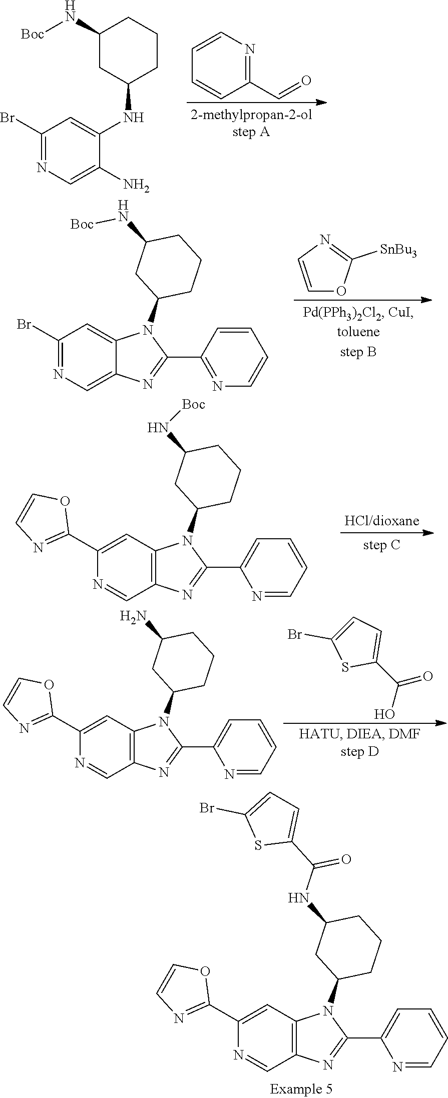

### Step A. Synthesis of tert-butyl ((1S,3R)-3-(6-bromo-2-(pyridin-2-yl)-1H-imidazo[4,5-c]pyridin-1-yl)cyclohexyl)carbamate

To a mixture of tert-butyl ((1S,3R)-3-((5-amino-2-bromopyridin-4-yl)amino)cyclohexyl)carbamate (300 mg, 779 μmol) in 2-methylpropan-2-ol (5 mL) was added pyridine-2-carbaldehyde (208 mg, 1.95 mmol). The reaction mixture was stirred at 100° C. for 12 hrs, and then concentrated to dryness. The residue was purified by silica gel column chromatography to give tert-butyl ((1S,3R)-3-(6-bromo-2-(pyridin-2-yl)-1H-imidazo[4,5-c]pyridin-1-yl)cyclohexyl)carbamate (130 mg, 34.1% yield). LC-MS: m/z 472.1 [M+1]⁺.

### Step B. Synthesis of tert-butyl ((1S,3R)-3-(6-(oxazol-2-yl)-2-(pyridin-2-yl)-1H-imidazo[4,5-c]pyridin-1-yl)cyclohexyl)carbamate

To a mixture of tert-butyl ((1S,3R)-3-(6-bromo-2-(pyridin-2-yl)-1H-imidazo[4,5-c]pyridin-1-yl)cyclohexyl)carbamate (110 mg, 233 μmol) and tributyl(oxazol-2-yl)stannane (167 mg, 466 μmol) in toluene (3 mL) were added Pd(PPh₃)₂Cl₂ (16.3 mg, 23.3 μmol) and CuI (13.3 mg, 69.9 μmol). The reaction mixture was stirred at 100° C. for 24 hrs under N₂. The reaction mixture was concentrated to dryness, and the residue purified by prep-HPLC to give tert-butyl ((1S,3R)-3-(6-(oxazol-2-yl)-2-(pyridin-2-yl)-1H-imidazo[4,5-c]pyridin-1-yl)cyclohexyl)carbamate (12 mg, 11.2% yield); LC-MS: m/z 461.2 [M+1]⁺.

### Step C. Synthesis of (1S,3R)-3-(6-(oxazol-2-yl)-2-(pyridin-2-yl)-1H-imidazo[4,5-c]pyridin-1-yl)cyclohexan-1-amine

A mixture of tert-butyl ((1S,3R)-3-(6-(oxazol-2-yl)-2-(pyridin-2-yl)-1H-imidazo[4,5-c]pyridin-1-yl)cyclohexyl)carbamate (12 mg, 26 μmol) in HCl/dioxane (4 M, 1 mL) was stirred at rt for 1 hr. The reaction mixture was concentrated to dryness to give (1S,3R)-3-(6-(oxazol-2-yl)-2-(pyridin-2-yl)-1H-imidazo[4,5-c]pyridin-1-yl)cyclohexan-1-amine (12 mg, HCl salt). LC-MS: m/z 361.2 [M+1]⁺.

### Step D. Synthesis of 5-bromo-N-((1S,3R)-3-(6-(oxazol-2-yl)-2-(pyridin-2-yl)-1H-imidazo[4,5-c]pyridin-1-yl)cyclohexyl)thiophene-2-carboxamide (Example 5)

To a solution of (1S,3R)-3-(6-(oxazol-2-yl)-2-(pyridin-2-yl)-1H-imidazo[4,5-c]pyridin-1-yl)cyclohexan-1-amine (12 mg, 30 μmol, HCl salt) and 5-bromothiophene-2-carboxylic acid (6.89 mg, 33.3 μmol) in DMF (1 mL) were added DIEA (19.5 mg, 151 μmol) and HATU (17.3 mg, 45.35 μmol). The reaction mixture was stirred at rt for 12 hrs, and then concentrated to dryness. The residue was purified by prep-HPLC to give Example 5 (7.2 mg, 43.1% yield). ¹H NMR (400 MHz, CDCl₃) δ 9.20 (s, 1H), 8.74 (d, 1H), 8.45 (s, 1H), 8.29 (d, 1H), 7.95-7.83 (m, 2H), 7.45 (dd, 1H), 7.33 (s, 1H), 7.23 (d, 1H), 7.01 (d, 1H), 6.08 (br d, 1H), 5.84 (br d, 1H), 4.23-4.11 (m, 1H), 2.47 (br d, 1H), 2.37-2.15 (m, 4H), 2.05 (br dd, 1H), 1.58-1.38 (m, 2H); LC-MS: m/z 551.2 [M+1]⁺.

### Preparation of Example 91 Via General Scheme IV (Method A)

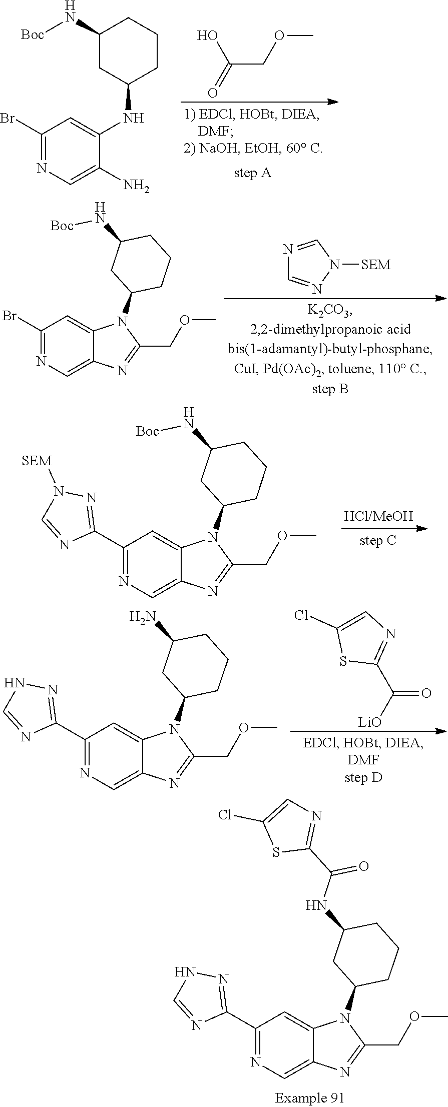

### Step A. Synthesis of tert-butyl N-[(1S,3R)-3-[6-bromo-2-(methoxymethyl)imidazo[4,5-c]pyridin-1-yl]cyclohexyl]carbamate

To a solution of tert-butyl N-[(1S,3R)-3-[(5-amino-2-bromo-4-pyridyl)amino]cyclohexyl]carbamate (400 mg, 1.04 mmol) and 2-methoxyacetic acid (374 mg, 4.15 mmol) in DMF (8 mL) were added EDCI (398 mg, 2.08 mmol), DIEA (268 mg, 2.08 mmol) and HOBt (281 mg, 2.08 mmol). The reaction mixture was stirred at rt for 48 hrs, then diluted with H₂O (20 mL), and the aqueous portion extracted with ethyl acetate (50 mL×3). The combined organic layers were dried over Na₂SO₄, filtered, and concentrated to dryness. The residue was purified by silica gel column chromatography to give tert-butyl N-[(1S,3R)-3-[[2-bromo-5-[(2-methoxyacetyl)amino]-4-pyridyl]amino]cyclohexyl]carbamate (380 mg, 80.0% yield). LC-MS: m/z 457.0 [M+H]⁺

To a solution of tert-butyl N-[(1S,3R)-3-[[2-bromo-5-[(2-methoxyacetyl)amino]-4-pyridyl]amino]cyclohexyl]carbamate (340 mg, 743 μmol) in EtOH (4 mL) was added NaOH (2 M, 4 mL). The reaction mixture was stirred at 60° C. for 16 hrs, then allowed to cool. The reaction mixture was diluted with H₂O (30 mL), and the aqueous portion extracted with EtOAc (50 mL×3). The combined organic layers were dried over Na₂SO₄, filtered, and concentrated to dryness. The residue was purified by silica gel column chromatography to give tert-butyl N-[(1S,3R)-3-[6-bromo-2-(methoxymethyl)imidazo[4,5-c]pyridine-1-yl]cyclohexyl]carbamate (170 mg, 52.1% yield). LC-MS: m/z 441.1 [M+H]⁺.

### Step B. Synthesis of tert-butyl N-[(1S,3R)-3-[2-(methoxymethyl)-6-[1-(2-trimethylsilylethoxy methyl)-1,2,4-triazol-3-yl]imidazo[4,5-c]pyridine-1-yl]cyclohexyl]carbamate

To a solution of tert-butyl N-[(1S,3R)-3-[6-bromo-2-(methoxymethyl)imidazo[4,5-c]pyridine-1-yl]cyclohexyl]carbamate (150 mg, 341 μmol) and trimethyl-[2-(1,2,4-triazol-1-ylmethoxy)ethyl]silane (136 mg, 683 μmol) in toluene (2 mL) were added Pd(OAc)₂ (30.7 mg, 137 μmol), bis(1-adamantyl)-butyl-phosphane (97.9 mg, 273 μmol), K₂CO₃ (142 mg, 1.02 mmol), 2,2-dimethylpropanoic acid (34.9 mg, 341 μmol) and CuI (39.0 mg, 205 μmol). The reaction mixture was degassed and purged with N₂ 3 times. The reaction mixture was then stirred at 110° C. for 16 hrs under N₂ atmosphere, then allowed to cool. The reaction mixture was quenched by H₂O (5 mL) at rt, diluted with H₂O (15 mL) and the aqueous portion extracted with EtOAc (20 mL×2). The combined organic layers were washed with brine (20 mL×3), dried over Na₂SO₄, filtered, and concentrated to dryness. The residue was purified by silica gel column chromatography to give tert-butyl N-[(1S,3R)-3-[2-(methoxymethyl)-6-[1-(2-trimethylsilylethoxy methyl)-1,2,4-triazol-3-yl]imidazo[4,5-c]pyridin-1-yl]cyclohexyl]carbamate (100 mg, 52.5% yield). LC-MS: m/z 558.1 [M+H]⁺.

### Step C. Synthesis of (1S,3R)-3-[2-(methoxymethyl)-6-(1H-1,2,4-triazol-3-yl)imidazo[4,5-c]pyridin-1-yl]cyclohexanamine

To a solution of tert-butyl N-[(1S,3R)-3-[2-(methoxymethyl)-6-[1-(2-trimethylsilylethoxymethyl)-1,2,4-triazol-3-yl]imidazo[4,5-c]pyridin-1-yl]cyclohexyl]carbamate (100 mg, 179 μmol) was added HCl/MeOH (4 M, 2 mL). The reaction mixture was stirred at rt for 16 hrs and then concentrated to dryness to give (1S,3R)-3-[2-(methoxymethyl)-6-(1H-1,2,4-triazol-3-yl)imidazo[4,5-c]pyridin-1-yl]cyclohexanamine (100 mg), which was used without further purification. LC-MS: m/z 328.2 [M+H]⁺

### Step D. Synthesis of 5-chloro-N-[(1S,3R)-3-[2-(methoxymethyl)-6-(1H-1,2,4-triazol-3-yl)imidazo[4,5-c]pyridin-1-yl]cyclohexyl]thiazole-2-carboxamide (Example 91)

To a solution of (1S,3R)-3-[2-(methoxymethyl)-6-(1H-1,2,4-triazol-3-yl)imidazo[4,5-c]pyridin-1-yl]cyclohexanamine (90 mg, 275 μmol) and (5-chlorothiazole-2-carbonyl)oxylithium (93.2 mg, 550 μmol) in DMF (2 mL) were added EDCI (79.1 mg, 412 μmol), DIEA (71.1 mg, 550 μmol) and HOBt (55.7 mg, 412 μmol). The reaction mixture was stirred at rt for 2 hrs, and then concentrated to dryness. The residue was purified by prep-HPLC to give Example 91 (13.7 mg, 10.3% yield). ¹H NMR (400 MHz, CDCl₃) δ 9.02 (s, 1H), 8.33 (s, 1H), 8.05 (s, 1H), 7.58 (s, 1H), 7.17 (br s, 1H), 4.79-4.68 (m, 2H), 4.79-4.68 (m, 2H), 4.19-4.08 (m, 1H), 3.38 (s, 3H), 2.39-2.23 (m, 2H), 2.20-2.11 (m, 2H), 2.06-1.94 (m, 2H), 1.24-1.13 (m, 2H); LC-MS: m/z 473.1 [M+H]⁺.

### Preparation of Examples 200 and 201 Via General Scheme IV (Method D)

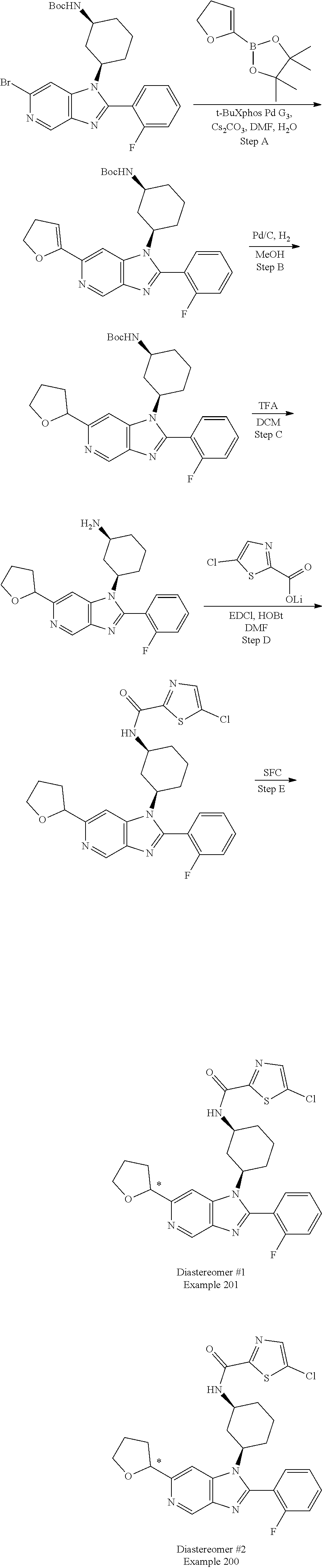

### Step A. Synthesis of tert-butyl ((1S,3R)-3-(6-(4,5-dihydrofuran-2-yl)-2-(2-fluorophenyl)-1H-imidazo[4,5-c]pyridin-1-yl)cyclohexyl)carbamate

To a solution of tert-butyl ((1S,3R)-3-(6-bromo-2-(2-fluorophenyl)-1H-imidazo[4,5-c]pyridin-1-yl)cyclohexyl)carbamate (300 mg, 615 μmol), prepared in accordance with procedure described for Example 68, and 2-(4,5-dihydrofuran-2-yl)-4,4,5,5-tetramethyl-1,3,2-dioxaborolane (180 mg, 922 μmol) in a mixture of DMF (10 mL) and water (3 mL) were added t-BuXphos Pd G3 (24 mg, 31 μmol) and Cs₂CO₃ (600 mg, 1.85 mmol). The reaction mixture was degassed under vacuum and purged with N₂ several times, then stirred at 80° C. for 4 hrs. The reaction mixture was cooled to rt and filtered through a Celite pad. Water (20 mL) was added to the filtrate, and the aqueous portion extracted with EtOAc (20 mL×3). The combined organic layers were washed with brine (20 mL), dried over Na₂SO₄, filtered, and concentrated to dryness. The residue was purified by flash column chromatography on silica gel to afford tert-butyl ((1S,3R)-3-(6-(4,5-dihydrofuran-2-yl)-2-(2-fluorophenyl)-1H-imidazo[4,5-c]pyridin-1-yl)cyclohexyl)carbamate (400 mg, 100% yield). LC-MS: m/z 479.2 [M+H]⁺.

### Step B. Synthesis of tert-butyl ((1S,3R)-3-(2-(2-fluorophenyl)-6-(tetrahydrofuran-2-yl)-1H-imidazo[4,5-c]pyridin-1-yl)cyclohexyl)carbamate

To a solution of tert-butyl ((1S,3R)-3-(6-(4,5-dihydrofuran-2-yl)-2-(2-fluorophenyl)-1H-imidazo[4,5-c]pyridin-1-yl)cyclohexyl)carbamate (170 mg, 355 μmol) in MeOH (4 mL) was added Pd/C (37.8 mg, 10% wt). The reaction mixture was degassed under vacuum and purged with H₂ several times. The reaction mixture was stirred at rt under H₂ atmosphere for 16 hrs. The reaction mixture was filtered through Celite©pad, and the filtrate concentrated to dryness. The residue was purified by flash column chromatography on silica gel to afford tert-butyl ((1S,3R)-3-(2-(2-fluorophenyl)-6-(tetrahydrofuran-2-yl)-1H-imidazo[4,5-c]pyridin-1-yl)cyclohexyl)carbamate (120 mg, 70% yield). LC-MS: m/z 481.3 [M+H]⁺.

### Step C. Synthesis of (1S,3R)-3-(2-(2-fluorophenyl)-6-(tetrahydrofuran-2-yl)-1H-imidazo[4,5-c]pyridin-1-yl)cyclohexan-1-amine

A solution of tert-butyl ((1S,3R)-3-(2-(2-fluorophenyl)-6-(tetrahydrofuran-2-yl)-1H-imidazo[4,5-c]pyridin-1-yl)cyclohexyl)carbamate (160 mg, 333 μmol) in a mixture of DCM (4 mL) and TFA (2 mL) was stirred at rt for 2 hrs. The reaction mixture was concentrated to dryness to afford (1S,3R)-3-(2-(2-fluorophenyl)-6-(tetrahydrofuran-2-yl)-1H-imidazo[4,5-c]pyridin-1-yl)cyclohexan-1-amine (120 mg, 95% yield). LC-MS: m/z 381.3 [M+H]⁺.

### Step D. Synthesis of 5-chloro-N-((1S,3R)-3-(2-(2-fluorophenyl)-6-(tetrahydrofuran-2-yl)-1H-imidazo[4,5-c]pyridin-1-yl)cyclohexyl)thiazole-2-carboxamide

To a mixture of HOBt (72.5 mg, 473 μmol) and EDCI (90.7 mg, 473 μmol) in DMF (5 mL) were added (1S,3R)-3-(2-(2-fluorophenyl)-6-(tetrahydrofuran-2-yl)-1H-imidazo[4,5-c]pyridin-1-yl)cyclohexan-1-amine (120 mg, 315 μmol), lithium 5-chlorothiazole-2-carboxylate (107 mg, 631 μmol) and pyridine (74.8 mg, 946 μmol). The reaction mixture was stirred at rt for 16 hrs. Water (30 mL) was added to the reaction mixture, and the aqueous portion extracted with EtOAc (3×10 mL). The combined organic layers were washed with brine (20 mL), dried over Na₂SO₄, filtered, and concentrated to dryness. The residue was purified by flash column chromatography on silica gel to afford 5-chloro-N-((1S,3R)-3-(2-(2-fluorophenyl)-6-(tetrahydrofuran-2-yl)-1H-imidazo[4,5-c]pyridin-1-yl)cyclohexyl)thiazole-2-carboxamide (80 mg, 48% yield). LC-MS: m/z 526.2 [M+H]⁺.

### Step E. Synthesis of 5-chloro-N-((1S,3R)-3-(2-(2-fluorophenyl)-6-(tetrahydrofuran-2-yl)-1H-imidazo[4,5-c]pyridin-1-yl)cyclohexyl)thiazole-2-carboxamide diastereomers (Examples 200 and 201)

5-Chloro-N-((1S,3R)-3-(2-(2-fluorophenyl)-6-(tetrahydrofuran-2-yl)-1H-imidazo[4,5-c]pyridin-1-yl)cyclohexyl)thiazole-2-carboxamide (80 mg, 0.15 mmol) was subjected to chiral SFC (column: DAICEL CHIRALPAK IG (250×4.6 mm, 5 um); mobile phase: [0.05% diethylamine MeOH];B %: 40%-40%,min) to afford Diastereomer #1 (25.6 mg, 32% yield, Rt=5.000 min) and Diastereomer #2 (24.3 mg, 31% yield, Rt=6.059 min).

Diastereomer #1 (Example 201): ¹H NMR (400 MHz, DMSO-d₆) δ 9.04 (d, 1H), 8.95 (s, 1H), 8.09 (s, 1H), 7.75-7.52 (m, 3H), 7.50-7.45 (m, 2H), 5.10-5.01 (m, 1H), 4.25-4.11 (m, 2H), 4.00-3.80 (m, 2H), 2.47-2.33 (m, 2H), 2.05-2.00 (m, 5H), 1.95-1.75 (m, 3H), 1.70-1.50 (m, 1H), 1.49-1.30 (m, 1H); LC-MS: m/z 526.2 [M+H]⁺; 92% ee.

Diastereomer #2 (Example 200): ¹H NMR (400 MHz, DMSO-d₆) δ 9.11 (d, 1H), 9.00 (s, 1H), 8.12 (s, 1H), 7.83-7.62 (m, 3H), 7.58-7.49 (m, 2H), 5.13-5.12 (m, 1H), 4.21-4.16 (m, 2H), 3.99-3.97 (m, 2H), 2.50-2.42 (m, 2H), 2.25-2.00 (m, 5H), 2.00-1.76 (m, 3H), 1.70-1.55 (m, 1H), 1.52-1.35 (m, 1H); LC-MS: m/z 526.2 [M+H]⁺; 95% ee.

### Synthesis of Example 202 Via General Scheme IV (Method H)

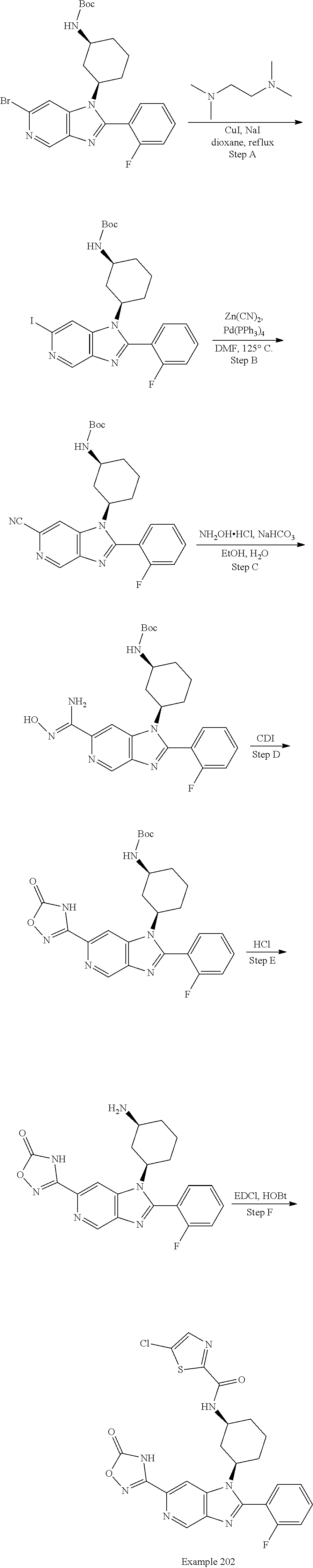

### Step A. Synthesis of tert-butyl ((1S,3R)-3-(2-(2-fluorophenyl)-6-iodo-1H-imidazo[4,5-c]pyridin-1-yl)cyclohexyl)carbamate

A mixture of tert-butyl ((1S,3R)-3-(6-bromo-2-(2-fluorophenyl)-1H-imidazo[4,5-c]pyridin-1-yl)cyclohexyl)carbamate (592 mg, 1.21 mmol), NaI (544 mg, 3.63 mmol), CuI (69.1 mg, 363 μmol) and N,N′-dimethylethane-1,2-diamine (64.0 mg, 726 μmol) in dioxane (6 mL) was degassed and purged with N₂ for 3 times, and then the reaction mixture was stirred at 110° C. for 12 hrs under N₂ atmosphere. Water (50 mL) was added to reaction mixture, and the aqueous portion extracted with DCM (100 mL×4). The combined organic layers were dried over Na₂SO₄, filtered, and concentrated to dryness to give tert-butyl ((1S,3R)-3-(2-(2-fluorophenyl)-6-iodo-1H-imidazo[4,5-c]pyridin-1-yl)cyclohexyl)carbamate (699 mg), which was used without further purification. LC-MS: m/z 537.2 [M+H]⁺.

### Step B. Synthesis of tert-butyl ((1S,3R)-3-(6-cyano-2-(2-fluorophenyl)-1H-imidazo[4,5-c]pyridin-1-yl)cyclohexyl)carbamate

A mixture of tert-butyl ((1S,3R)-3-(2-(2-fluorophenyl)-6-iodo-1H-imidazo[4,5-c]pyridin-1-yl)cyclohexyl)carbamate (650 mg, 1.21 mmol), Pd(PPh₃)₄ (140 mg, 121 μmol), and Zn(CN)₂ (498 mg, 4.24 mmol) in DMF (7 mL) was degassed and purged with N₂ for 3 times, and the reaction mixture was stirred at 125° C. for 16 hrs under N₂ atmosphere. The reaction mixture was cooled to rt, and DCM (100 mL) added. The organic portion was washed with water (100 mL) and brine (100 mL), dried over Na₂SO₄, filtered and concentrated to dryness. The residue was purified by flash silica gel chromatography to give tert-butyl ((1S,3R)-3-(6-cyano-2-(2-fluorophenyl)-1H-imidazo[4,5-c]pyridin-1-yl)cyclohexyl)carbamate (336 mg, 63.6% yield). LC-MS: m/z 436.2 [M+H]⁺.

### Step C. Synthesis of tert-butyl ((1S,3R)-3-(2-(2-fluorophenyl)-6-((Z)—N′-hydroxycarbamimidoyl)-1H-imidazo[4,5-c]pyridin-1-yl)cyclohexyl)carbamate

Hydroxylamine hydrochloride (95.7 mg, 1.38 mmol) and NaHCO₃ (174 mg, 2.07 mmol) were added to H₂O (1.2 mL) and the reaction mixture was stirred for 15 min. The resulting solution was then added to tert-butyl N-[(1S,3R)-3-[6-cyano-2-(2-fluorophenyl)imidazo[4,5-c]pyridin-1-yl]cyclohexyl]carbamate (300 mg, 689 μmol) in EtOH (3 mL) at rt. The reaction mixture was stirred at 100° C. for 1 hr. The reaction mixture was filtered, and the filtrate was concentrated to dryness to give tert-butyl ((1S,3R)-3-(2-(2-fluorophenyl)-6-((Z)—N′-hydroxycarbamimidoyl)-1H-imidazo[4,5-c]pyridin-1-yl)cyclohexyl) carbamate (324 mg), which was used without further purification. LC-MS: m/z 469.3 [M+H]⁺.

### Step D. Synthesis of tert-butyl ((1S,3R)-3-(2-(2-fluorophenyl)-6-(5-oxo-4,5-dihydro-1,2,4-oxadiazol-3-yl)-1H-imidazo[4,5-c]pyridin-1-yl)cyclohexyl)carbamate

To a solution of tert-butyl ((1S,3R)-3-(2-(2-fluorophenyl)-6-((Z)—N′-hydroxycarbamimidoyl)-1H-imidazo[4,5-c]pyridin-1-yl)cyclohexyl)carbamate (250 mg, 534 μmol) and di(imidazol-1-yl)methanone (433 mg, 2.67 mmol) in MeCN (5 mL) was added DBU (406 mg, 2.67 mmol). The reaction mixture was stirred at rt for 1 hr. To the reaction mixture was added a mixture of H₂O/EtOAc (50 mL/50 mL). The aqueous portion was extracted with EtOAc (50 mL×3). The combined organic layers were concentrated to dryness. The residue was purified by flash silica gel chromatography to give tert-butyl ((1S,3R)-3-(2-(2-fluorophenyl)-6-(5-oxo-4,5-dihydro-1,2,4-oxadiazol-3-yl)-1H-imidazo[4,5-c]pyridin-1-yl)cyclohexyl)carbamate (285 mg, 88.6% yield). LC-MS: m/z 495.2 [M+H]⁺.

### Step E. Synthesis of 3-(1-((1R,3S)-3-aminocyclohexyl)-2-(2-fluorophenyl)-1H-imidazo[4,5-c]pyridin-6-yl)-1,2,4-oxadiazol-5(4H)-one

To a solution of tert-butyl ((1S,3R)-3-(2-(2-fluorophenyl)-6-(5-oxo-4,5-dihydro-1,2,4-oxadiazol-3-yl)-1H-imidazo[4,5-c]pyridin-1-yl)cyclohexyl)carbamate (285 mg, 576 μmol) in HCl/MeOH (3 mL). The reaction mixture was stirred at rt for 1 hr and then concentrated to dryness to give 3-(1-((1R,3S)-3-aminocyclohexyl)-2-(2-fluorophenyl)-1H-imidazo[4,5-c]pyridin-6-yl)-1,2,4-oxadiazol-5(4H)-one (272 mg, 99.7% yield), which was used without further purification. LC-MS: m/z 395.1 [M+H]⁺.

### Step F. Synthesis of 5-chloro-N-((1S,3R)-3-(2-(2-fluorophenyl)-6-(5-oxo-4,5-dihydro-1,2,4-oxadiazol-3-yl)-1H-imidazo[4,5-c]pyridin-1-yl)cyclohexyl)thiazole-2-carboxamide (Example 202)

To a solution of 5-chlorothiazole-2-carboxylic acid (141 mg, 828 μmol, Li salt), 3-(1-((1R,3S)-3-aminocyclohexyl)-2-(2-fluorophenyl)-1H-imidazo[4,5-c]pyridin-6-yl)-1,2,4-oxadiazol-5(4H)-one (272 mg, 690 μmol) in DMF (3 mL) was added EDCI (264 mg, 1.38 mmol), DIEA (267 mg, 2.07 mmol, 360 μL) and HOBt (186 mg, 1.38 mmol). The reaction mixture was stirred at 50° C. for 16 hrs. To the reaction mixture was added a mixture of H₂O/EtOAc (20 mL/20 mL). The aqueous portion was extracted with EtOAc (20 mL×3). The combined organic layers were concentrated to dryness. The residue was first purified by flash silica gel chromatography and then further purified by prep-HPLC (column: Welch Xtimate C18 150*30 mm*5 um; mobile phase: [water (FA)-ACN]; B %: 48%-78%, 7 min) to give Example 202 (54.7 mg, 14.7% yield). ¹H NMR (400 MHz, CDCl₃) δ 9.05 (s, 1H), 8.24 (s, 1H), 7.61-7.58 (m, 3H), 7.36-7.34 (m, 1H), 7.28-7.25 (m, 1H), 7.12-7.09 (m, 1H), 4.22-4.19 (m, 1H), 3.98-3.96 (m, 1H), 2.33-2.31 (m, 1H), 2.17-1.99 (m, 5H), 1.44-1.41 (m, 2H); LC-MS: m/z 540.1 [M+H]⁺.

### Preparation of Example 203 Via General Scheme IV (Method G)

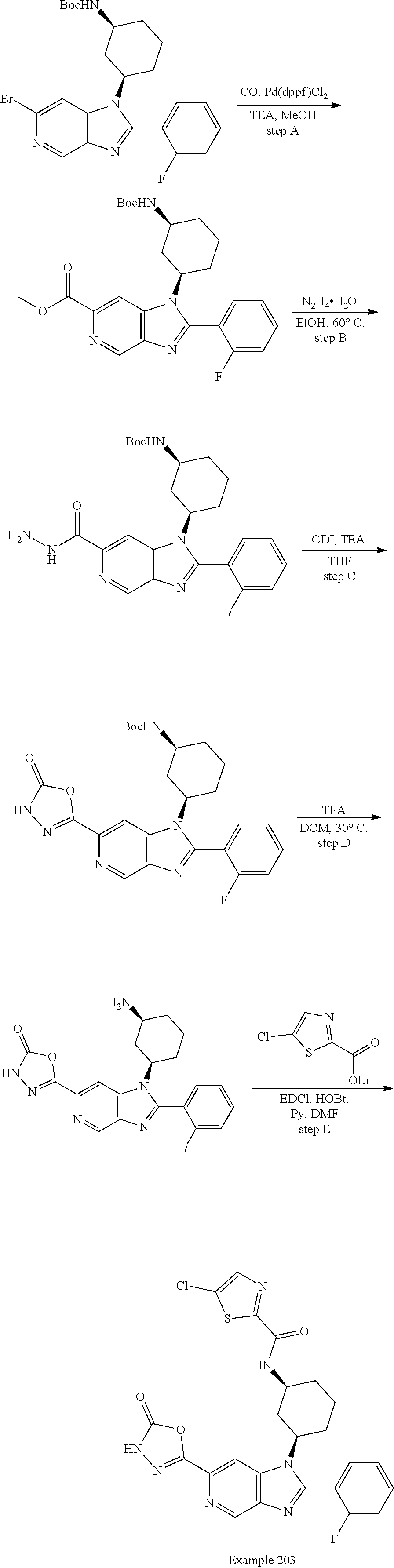

### Step A. Synthesis of methyl 1-((1R,3S)-3-((tert-butoxycarbonyl)amino)cyclohexyl)-2-(2-fluorophenyl)-1H-imidazo[4,5-c]pyridine-6-carboxylate

To a mixture of tert-butyl ((1S,3R)-3-(6-bromo-2-(2-fluorophenyl)-1H-imidazo[4,5-c]pyridin-1-yl)cyclohexyl)carbamate (500 mg, 1.02 mmol) in MeOH (10 mL) were added Pd(dppf)Cl₂ (75 mg, 102 μmol) and TEA (310 mg, 3.07 mmol). The reaction mixture was degassed under vacuum and purged with CO several times, and then heated to 80° C. under CO atmosphere for 4 hrs. The reaction mixture was cooled to rt and filtered through a Celite pad. Water (20 mL) was added to the filtrate, and the aqueous portion extracted with EtOAc (20 mL×3). The combined organic layers were washed with brine (20 mL), dried over Na₂SO₄, filtered, and concentrated to dryness. The residue was purified by flash column chromatography on silica gel to afford methyl 1-((1R,3S)-3-((tert-butoxycarbonyl)amino)cyclohexyl)-2-(2-fluorophenyl)-1H-imidazo[4,5-c]pyridine-6-carboxylate (400 mg, 83.6% yield). ¹H NMR (400 MHz, CDCl₃) δ 9.22 (s, 1H), 8.44 (s, 1H), 7.67-7.60 (m, 2H), 7.41-7.37 (m, 1H), 7.31-7.26 (m, 1H), 4.52 (s, 1H), 4.25-4.15 (m, 1H), 4.08 (s, 3H), 3.54 (s, 1H), 2.40-2.30 (m, 1H), 2.18-1.99 (m, 6H), 1.41 (s, 9H), 1.35-1.25 (m, 1H); LC-MS: m/z 469.2 [M+H]⁺.

### Step B. Synthesis of tert-butyl ((1S,3R)-3-(2-(2-fluorophenyl)-6-(hydrazinecarbonyl)-1H-imidazo[4,5-c]pyridin-1-yl)cyclohexyl)carbamate

To a solution of methyl 1-((1R,3S)-3-((tert-butoxycarbonyl)amino)cyclohexyl)-2-(2-fluorophenyl)-1H-imidazo[4,5-c]pyridine-6-carboxylate (500 mg, 1.07 mmol) in EtOH (10 mL) was added hydrazine (855 mg, 40% wt, 10.7 mmol). The reaction mixture was stirred at 60° C. for 3 hrs and then concentrated to dryness. The residue was purified by C18 column chromatography to give tert-butyl ((1S,3R)-3-(2-(2-fluorophenyl)-6-(hydrazinecarbonyl)-1H-imidazo[4,5-c]pyridin-1-yl)cyclohexyl)carbamate (210 mg, 42.0% yield). LC-MS: m/z 469.2 [M+H]⁺.

### Step C. Synthesis of tert-butyl ((1S,3R)-3-(2-(2-fluorophenyl)-6-(5-oxo-4,5-dihydro-1,3,4-oxadiazol-2-yl)-1H-imidazo[4,5-c]pyridin-1-yl)cyclohexyl)carbamate

To a solution of tert-butyl ((1S,3R)-3-(2-(2-fluorophenyl)-6-(hydrazinecarbonyl)-1H-imidazo[4,5-c]pyridin-1-yl)cyclohexyl)carbamate (200 mg, 427 μmol) and TEA (52 mg, 512 μmol) in THF (4 mL) was added CDI (90 mg, 555 μmol). The reaction mixture was stirred at rt for 5 hrs. Water (10 mL) was added to the reaction mixture, and the aqueous portion extracted with EtOAc (10 mL×3). The combined organic layers were washed with water (15 mL) and brine (15 mL), dried over Na₂SO₄, filtered, and concentrated to dryness. The residue was purified by C18 column chromatography to give tert-butyl ((1S,3R)-3-(2-(2-fluorophenyl)-6-(5-oxo-4,5-dihydro-1,3,4-oxadiazol-2-yl)-1H-imidazo[4,5-c]pyridin-1-yl)cyclohexyl)carbamate (120 mg, 56.8% yield). ¹H NMR (400 MHz, DMSO-d₆) δ 12.75 (s, 1H), 9.14 (s, 1H), 8.22 (s, 1H), 7.77-7.72 (m, 2H), 7.54-7.45 (m, 2H), 6.98-6.90 (m, 1H), 4.25-4.15 (m, 1H), 2.06-1.89 (m, 3H), 1.81-1.78 (m, 3H), 1.40 (s, 9H), 1.34-1.18 (m, 2H); LC-MS: m/z 495.2 [M+H]⁺.

### Step D. Synthesis of 5-(1-((1R,3S)-3-aminocyclohexyl)-2-(2-fluorophenyl)-1H-imidazo[4,5-c]pyridin-6-yl)-1,3,4-oxadiazol-2(3H)-one

To a solution of tert-butyl ((1S,3R)-3-(2-(2-fluorophenyl)-6-(5-oxo-4,5-dihydro-1,3,4-oxadiazol-2-yl)-1H-imidazo[4,5-c]pyridin-1-yl)cyclohexyl)carbamate (120 mg, 243 μmol) in DCM (5 mL) was added TFA (138 mg, 1.21 mmol). The reaction mixture was stirred at 30° C. for 16 hrs. The reaction mixture was concentrated to dryness to give 5-(1-((1R,3S)-3-aminocyclohexyl)-2-(2-fluorophenyl)-1H-imidazo[4,5-c]pyridin-6-yl)-1,3,4-oxadiazol-2(3H)-one (130 mg, quant.), which was used without further purification. LC-MS: m/z 395.2 [M+H]⁺

### Step E. Synthesis of 5-chloro-N-((1S,3R)-3-(2-(2-fluorophenyl)-6-(5-oxo-4,5-dihydro-1,3,4-oxadiazol-2-yl)-1H-imidazo[4,5-c]pyridin-1-yl)cyclohexyl)thiazole-2-carboxamide (Example 203)

To a solution of 5-(1-((1R,3S)-3-aminocyclohexyl)-2-(2-fluorophenyl)-1H-imidazo[4,5-c]pyridin-6-yl)-1,3,4-oxadiazol-2(3H)-one (120 mg, 304 μmol) in DMF (5 mL) were added lithium 5-chlorothiazole-2-carboxylate (67 mg, 396 mol), HOBt (61 mg, 396 μmol), EDCI (76 mg, 396 μmol) and pyridine (72 mg, 913 μmol). The reaction mixture was stirred at rt for 16 hrs. Water (15 mL) was added to the reaction mixture, and the aqueous portion extracted with EtOAc (15 mL×3). The combined organic layers were washed with water and brine and then concentrated to dryness. The residue was purified by prep-HPLC to afford Example 203 (21 mg, 1300 yield). ¹H NMR (400 MHz, DMSO-d₆) δ 12.76 (s, 1H), 9.14 (s, 1H), 9.02 (d, 1H), 8.27 (s, 1H), 8.06 (s, 1H), 7.77-7.71 (m, 2H), 7.55-7.45 (m, 2H), 4.30-4.22 (m, 1H), 3.99-3.32 (m, 1H), 2.54-2.51 (m, 1H), 2.07-2.04 (m, 2H), 1.92-1.76 (m, 3H), 1.59-1.55 (m, 1H), 1.50-1.25 (m, 1H); LC-MS: m/z 540.1 [M+H]⁺.

General Scheme IV set forth above was used to synthesize the following compounds by using appropriate starting materials:

| Ex. | | | |
|---|---|---|---|
| No. | Structure | ¹H NMR | LC-MS and % ee |
| 3 | 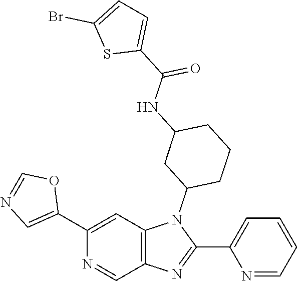 [SMILES: O=C(NC1CCCC(n2c(-c3ccccn3)nc3cnc(-c4cnco4)cc32)C1)c1ccc(Br)s1] | (400 MHz, CDCl₃) δ 9.12 (s, 1 H), 8.74 (d, 1 H), 8.28 (d, 1 H), 8.01-7.85 (m, 3 H), 7.73 (s, 1 H), 7.47-7.40 (m, 1 H), 7.20 (d, 1 H), 7.00 (d, 1 H), 5.95 (br d, 1 H), 5.81 (br s, 1 H), 4.22-4.09 (m, 1 H), 2.51 (br d, 1 H), 2.29- 2.18 (m, 4 H), 2.08 (br d, 1 H), 1.65 (br d, 1 H), 1.63 (br s, 1 H). | m/z 549.2 [M + H]⁺ |
| | (relative stereochemistry at | | |
| | centers 1 and 3 of cyclohexyl | | |
| | is cis) | | |
| 6 | 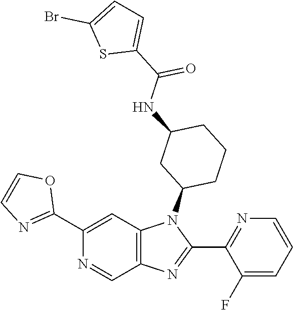 [SMILES: O=C(N[C@H]1CCC[C@@H](n2c(-c3ncccc3F)nc3cnc(-c4ncco4)cc32)C1)c1ccc(Br)s1] | (400 MHz, CDCl₃) δ 9.24 (s, 1 H), 8.62 (br d, 1 H), 8.45 (s, 1 H), 7.86 (s, 1 H), 7.74-7.64 (m, 1 H), 7.59- 7.49 (m, 1 H), 7.33 (s, 1 H), 7.23 (br s, 1 H), 7.00 (d, 1 H), 6.15 (br d, 1 H), 4.82 (br s, 1 H), 4.13-4.02 (m, 1 H), 2.45 (br d, 1 H), 2.37- 2.09 (m, 4 H), 2.06-1.97 (m, 1 H), 1.56-1.35 (m, 2 H). | m/z 567.2 [M + H]⁺ |
| 7 | 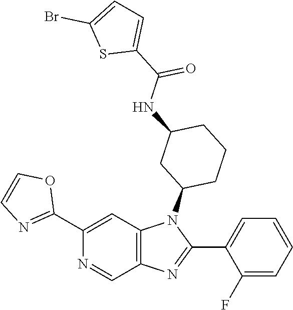 [SMILES: O=C(N[C@H]1CCC[C@@H](n2c(-c3ccccc3F)nc3cnc(-c4ncco4)cc32)C1)c1ccc(Br)s1] | (400 MHz, CDCl₃) δ 9.24 (s, 1 H), 8.44 (s, 1 H), 7.90 (s, 1 H), 7.71-7.62 (m, 2 H), 7.45-7.30 (m, 4 H), 7.26- 7.24 (m, 1 H), 7.05 (d, 1 H), 4.32-4.,2 (m, 1 H), 4.14- 4.01 (m, 1 H), 2.43-2.24 (m, 3 H), 2.20-2.01 (m, 3 H), 1.53-1.41 (m, 2 H). | m/z 566.2 [M + H]⁺ |
| 8 | 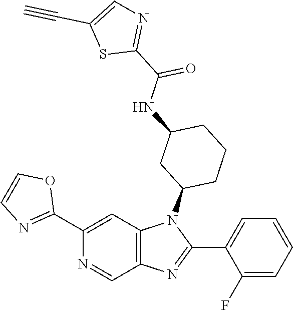 [SMILES: C#Cc1cnc(C(=O)N[C@H]2CCC[C@@H](n3c(-c4ccccc4F)nc4cnc(-c5ncco5)cc43)C2)s1] | (400 MHz, CDCl₃) δ 9.20 (s, 1 H), 8.41 (s, 1 H), 7.87 (d, 2 H), 7.69-7.57 (m, 2 H), 7.39 (t, 1 H), 7.34-7.27 (m, 2 H), 7.22 (br s, 1 H), 4.32- 4.17 (m, 1 H), 4.10-3.96 (m, 1 H), 3.54 (s, 1 H), 2.43- 2.25 (m, 3 H), 2.18-2.02 (m, 3 H), 1.51 (br t, 2 H). | m/z 513.2 [M + H]⁺ |
| 9 | 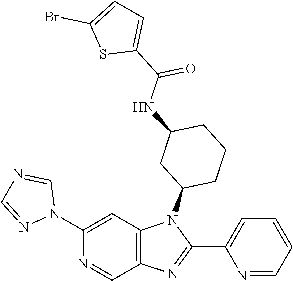 [SMILES: O=C(N[C@H]1CCC[C@@H](n2c(-c3ccccn3)nc3cnc(-n4cncn4)cc32)C1)c1ccc(Br)s1] | (400 MHz, CD₃OD) δ 9.42 (s, 1 H), 8.94 (s, 1 H), 8.85 (d, 1 H), 8.35-8.18 (m, 3 H), 8.08 (td, 1 H), 7.62 (dd, 1 H), 7.50 (d, 1 H), 7.13 (d, 1 H), 5.61 (s, 1 H), 4.05 (br s, 1 H), 2.53-2.30 (m, 3 H), 2.23-2.03 (m, 3 H), 1.61 (br t, 2 H). | m/z 551.2 [M + H]⁺ |
| 10 | 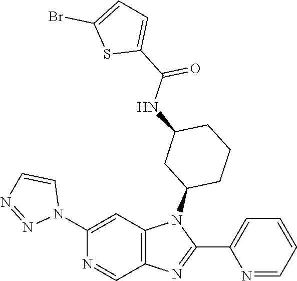 [SMILES: O=C(N[C@H]1CCC[C@@H](n2c(-c3ccccn3)nc3cnc(-n4ccnn4)cc32)C1)c1ccc(Br)s1] | (400 MHz, CD₃OD) δ 9.00 (s, 1 H), 8.90-8.84 (m, 2 H), 8.57 (s, 1 H), 8.24 (br d, 1 H), 8.10 (br t, 1 H), 7.98 (s, 1 H), 7.60-7.68 (m, 1 H), 7.51 (d, 1 H), 7.14 (d, 1 H), 5.69-5.57 (m, 1 H), 4.07 (br s, 1 H), 2.58-2.32 (m, 3 H), 2.26-2.17 (m, 1 H), 2.13- 2.05 (m, 2 H), 1.65-1.59 (m, 2 H). | m/z 551.2 [M + H]⁺ |
| 11 | 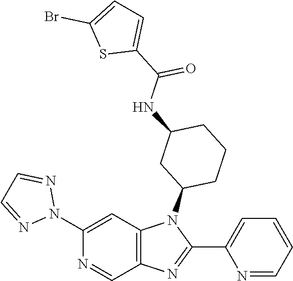 [SMILES: O=C(N[C@H]1CCC[C@@H](n2c(-c3ccccn3)nc3cnc(-n4nccn4)cc32)C1)c1ccc(Br)s1] | (400 MHz, CD₃OD) δ 8.96 (br s, 1 H), 8.85 (br d, 1 H), 8.50 (br s, 1 H), 8.24 (br d, 1 H), 8.12-8.06 (m, 3 H), 7.66-7.60 (m, 1 H), 7.50 (d, 1 H), 7.13 (d, 1 H), 5.64 (br t, 1 H), 4.06 (br d, 1 H), 2.55-2.30 (m, 3 H), 2.25- 2.17 (m, 1 H), 2.09 (br d, 2 H), 1.60 (br t, 2 H). | m/z 551.2 [M + H]⁺ |
| 18 | 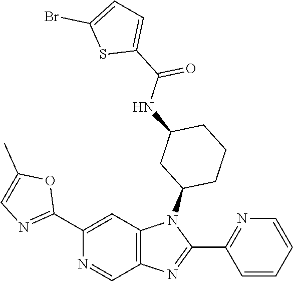 [SMILES: Cc1cnc(-c2cc3c(cn2)nc(-c2ccccn2)n3[C@@H]2CCC[C@H](NC(=O)c3ccc(Br)s3)C2)o1] | (400 MHz, DMSO-d₆) δ 9.11 (s, 1 H), 8.82 (d, 1 H), 8.62 (d, 1 H), 8.42 (s, 1 H), 8.24 (d, 1 H), 8.09 (td, 1 H), 7.63 (dd, 1 H), 7.58 (d, 1 H), 7.26 (d, 1 H), 7.10 (d, 1 H), 5.62 (br t, 1 H), 3.94 (br s, 1 H), 2.44 (s, 3 H), 2.38 (br d, 1 H), 2.27-2.15 (m, 2 H), 2.06 (br d, 1 H), 1.94 (br s, 2 H), 1.52-1.43 (m, 2 H). | m/z 565.3 [M + H]⁺ |
| 31 | 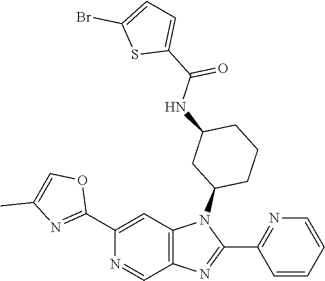 [SMILES: Cc1coc(-c2cc3c(cn2)nc(-c2ccccn2)n3[C@@H]2CCC[C@H](NC(=O)c3ccc(Br)s3)C2)n1] | (400 MHz, CD₃OD) δ 9.06 (br s, 1 H), 8.85 (br s, 1 H), 8.59 (s, 1 H), 8.23 (br d, 1 H), 8.11-8.03 (m, 1 H), 7.61 (br d, 1 H), 7.50 (br d, 1 H), 7.12 (br d, 1 H), 7.07 (br s, 1 H), 5.63 (br s, 1 H), 4.04 (br s, 1 H), 3.86 (s, 1 H), 2.51 (s, 3 H), 2.39 (br s, 2 H), 2.19 (br s, 1 H), 2.08 (br d, 2 H), 1.61 (br s, 2 H). | m/z 565.2 [M + H]⁺ |
| 55 | 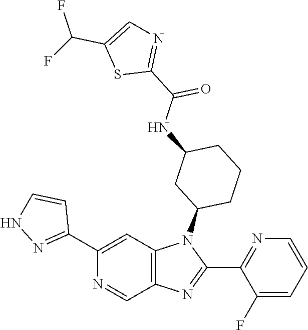 [SMILES: O=C(N[C@H]1CCC[C@@H](n2c(-c3ncccc3F)nc3cnc(-c4cc[nH]n4)cc32)C1)c1ncc(C(F)F)s1] | (400 MHz, CDCl₃) δ 9.20 (br s, 1 H), 8.67 (br d, 1 H), 8.10-7.97 (m, 2 H), 7.73 (br t, 2 H), 7.58 (br dd, 1 H), 7.40-7.33 (m, 1 H), 7.11- 6.74 (m, 2 H), 4.85 (br s, 1 H), 4.12 (br s, 1 H), 2.60- 2.48 (m, 1 H), 2.40-2.18 (m, 4 H), 2.14-2.06 (m, 1 H), 1.62-1.49 (m, 2 H). | m/z 539.1 [M + H]⁺ |
| 77 | 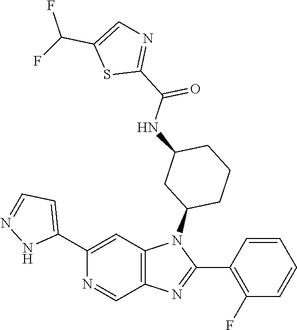 [SMILES: O=C(N[C@H]1CCC[C@@H](n2c(-c3ccccc3F)nc3cnc(-c4ccn[nH]4)cc32)C1)c1ncc(C(F)F)s1] | (400 MHz, CD₃OD) δ 8.99 (s, 1 H), 8.48 (br s, 1 H), 8.13 (t, 1 H), 7.79-7.69 (m, 3 H), 7.49-7.42 (m, 2 H), 7.29-7.02 (m, 2 H), 4.61 (br s, 1 H), 4.29 (br t, 1 H), 4.04-3.98 (m, 1 H), 2.56 (q, 1 H), 2.45-2.34 (m, 1 H), 2.27 (br d, 1 H), 2.04 (br d, 3 H), 1.75-1.64 (m, 1 H), 1.55-1.46 (m, 1 H). | m/z 538.2 [M + H]⁺ |
| 78 | 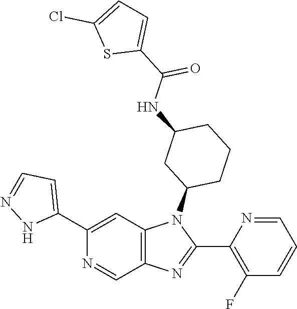 [SMILES: O=C(N[C@H]1CCC[C@@H](n2c(-c3ncccc3F)nc3cnc(-c4ccn[nH]4)cc32)C1)c1ccc(Cl)s1] | (400 MHz, CD₃OD) δ 9.05 (s, 1 H), 8.71 (d, 1 H), 8.48 (br s, 1 H), 7.98-7.91 (m, 1 H), 7.83-7.72 (m, 2 H), 7.55 (d, 1 H), 7.07-6.98 (m, 2 H), 4.75 (br s, 1 H), 3.98 (br s, 1 H), 2.53-2.32 (m, 3 H), 2.19-2.01 (m, 3 H), 1.65- 1.47 (m, 2 H). | m/z 522.2 [M + H]⁺ |
| 81 | 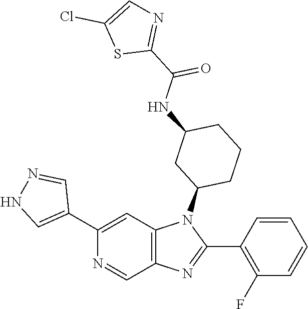 [SMILES: O=C(N[C@H]1CCC[C@@H](n2c(-c3ccccc3F)nc3cnc(-c4cn[nH]c4)cc32)C1)c1ncc(Cl)s1] | (400 MHz, CD₃OD) δ 8.93 (s, 1 H), 8.30 (br s, 2 H), 8.08 (s, 1 H), 7.80 (s, 1 H), 7.75-7.66 (m, 2 H), 7.49- 7.40 (m, 2 H), 4.27 (br t, 1 H), 4.00-3.93 (m, 1 H), 2.58 (q, 1 H), 2.33-2.20 (m, 2 H), 2.00 (br s, 3 H), 1.73-1.60 (m, 1 H), 1.52-1.42 (m, 1 H). | m/z 522.2 [M + H]⁺ |
| 86 | 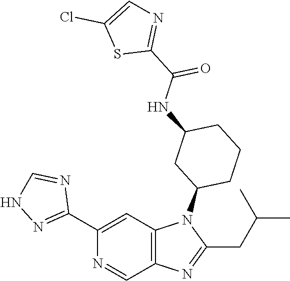 [SMILES: CC(C)Cc1nc2cnc(-c3nc[nH]n3)cc2n1[C@@H]1CCC[C@H](NC(=O)c2ncc(Cl)s2)C1] | (400 MHz, CDCl₃) δ 9.18 (br s, 1 H), 8.57 (br s, 1 H), 8.45-8.32 (m, 1 H), 7.67 (s, 1 H), 7.43 (br d, 1 H), 4.46 (br s, 1 H), 4.32-4.19 (m, 1 H), 2.90 (br d, 2 H), 1.47- 1.99 (m, 6 H), 1.78-1.63 (m, 2 H), 1.12 (br d, 6 H). | m/z 485.2 [M + H]⁺ |
| 87 | 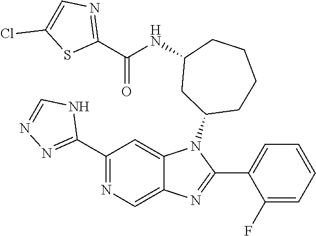 [SMILES: O=C(N[C@@H]1CCCC[C@H](n2c(-c3ccccc3F)nc3cnc(-c4nnc[nH]4)cc32)C1)c1ncc(Cl)s1] | (400 MHz, CDCl₃) δ 9.13 (s, 1 H), 8.42 (s, 1 H), 8.11 (s, 1 H), 7.71 (td, 1 H), 7.66-7.62 (m, 1 H), 7.61 (s, 1 H), 7.42 (td, 1 H), 7.34 (t, 1 H), 7.18 (br d, 1 H), 4.43 (br t, 1 H), 4.13 (br s, 1 H), 2.76-2.67 (m, 1 H), 2.55- 2.48 (m, 1 H), 2.44-2.41 (m, 1 H), 2.15-2.04 (m, 2 H), 1.90-1.77 (m, 5 H) | m/z 537.2 [M + H]⁺; (column: DAICEL CHIRALPAK IG (250 mm * 30 mm, 10 um); mobile phase: [0.1% NH₃H₂O IPA]; B%: 50%-50%, min); Rt = 2.541 min 100% ee |
| 88 | 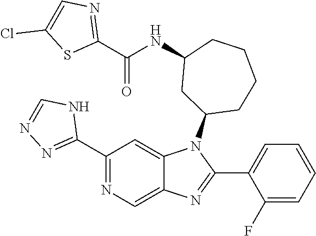 [SMILES: O=C(N[C@H]1CCCC[C@@H](n2c(-c3ccccc3F)nc3cnc(-c4nnc[nH]4)cc32)C1)c1ncc(Cl)s1] | (400 MHz, CDCl₃) δ 9.13 (s, 1 H), 8.42 (s, 1 H), 8.12 (s, 1 H), 7.71 (td, 1 H), 7.66-7.62 (m, 1 H), 7.61 (s, 1 H), 7.45-7.39 (m, 1 H), 7.33 (t, 1 H), 7.19 (br d, 1 H), 4.43 (br t, 1 H), 4.12 (m, 1 H), 2.76-2.67 (m, 1 H), 2.55-2.47 (m, 1 H), 2.44- 2.41 (m, 1 H), 2.15-2.05 (m, 3 H), 1.89-1.76 (m, 5 H) | m/z 537.2 [M + H]⁺; (column: DAICEL CHIRALPAK IG (250 mm * 30 mm, 10 um); mobile phase: [0.1% NH₃H₂O IPA]; B%: 50%-50%, min); Rt = 4.124 min 95.4% ee |
| 89 | 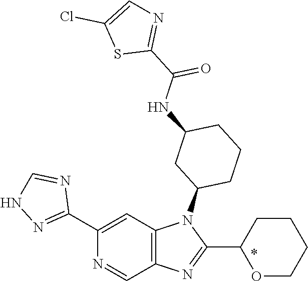 [SMILES: O=C(N[C@H]1CCC[C@@H](n2c(C3CCCCO3)nc3cnc(-c4nc[nH]n4)cc32)C1)c1ncc(Cl)s1] | (400 MHz, CDCl₃) δ 9.00 (s, 1 H), 8.32 (s, 1 H), 8.03 (s, 1 H), 7.57 (s, 1 H), 7.18 (br d, 1 H), 4.67 (br dd, 2 H), 4.20-4.02 (m, 2 H), 3.69-3.58 (m, 1 H), 2.39 (br d, 1 H), 2.27-1.91 (m, 9 H), 1.58-1.39 (m, 4 H). | m/z 513.2 [M + H]⁺; (column: DAICEL CHIRALPAK AD(250 mm * 30 mm, 10 um); mobile phase: [0.1% NH₃H₂O MeOH]; B%: 45%-45%, min); Rt = 2.638 min 100% ee |
| 90 | 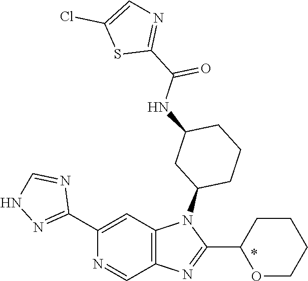 [SMILES: O=C(N[C@H]1CCC[C@@H](n2c(C3CCCCO3)nc3cnc(-c4nc[nH]n4)cc32)C1)c1ncc(Cl)s1] | (400 MHz, CDCl₃) δ 9.01 (s, 1 H), 8.31 (s, 1 H), 8.03 (s, 1 H), 7.58 (s, 1 H), 7.23- 7.20 (m, 1 H), 4.72-4.59 (m, 2 H), 4.17-3.97 (m, 2 H), 3.64 (br d, 1 H), 2.36-2.24 (m, 2 H), 2.20-1.97 (m, 8 H), 1.56-1.49 (m, 4 H). | m/z 513.2 [M + H]⁺; (column: DAICEL CHIRALPAK AD(250 mm * 30 mm, 10 um); mobile phase: [0.1% NH₃H₂O MeOH]; B%: 45%-45%, min); Rt = 5.178 min 100% ee |
| 102 | 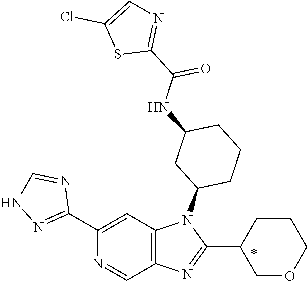 [SMILES: O=C(N[C@H]1CCC[C@@H](n2c(C3CCCOC3)nc3cnc(-c4nc[nH]n4)cc32)C1)c1ncc(Cl)s1] | (400 MHz, CD₃OD) δ 8.85 (br s, 1 H), 8.43 (br s, 1 H), 8.22 (br s, 1 H), 7.71 (s, 1 H), 4.68-4.42 (m, 1 H), 4.23-3.87 (m, 4 H), 3.67 (br t, 0.7 H), 3.56-3.36 (m, 1.3 H), 2.49-2.35 (m, 0.6 H), 2.33-2.09 (m, 4 H), 2.05- 1.80 (m, 5.5 H), 1.68 (br dd, 2 H) | m/z 513.2 [M + H]⁺; (column: DAICEL CHIRALPAK IC(250 mm * 30 mm, 10 um); mobile phase: [0.1% NH₃H₂O EtOH]; B%: 55%-55%, min) Rt = 0.912 min 97.2% ee |
| 103 | 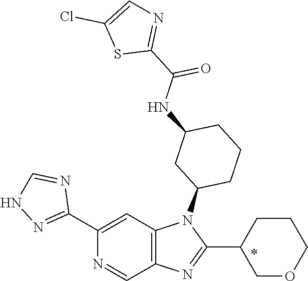 [SMILES: O=C(N[C@H]1CCC[C@@H](n2c(C3CCCOC3)nc3cnc(-c4nc[nH]n4)cc32)C1)c1ncc(Cl)s1] | (400 MHz, CD₃OD) δ 8.85 (s, 1 H), 8.43 (s, 1 H), 8.31- 8.03 (m, 1 H), 7.71 (s, 1 H), 4.61 (br t, 1.4 H), 4.19-4.02 (m, 1.7 H), 3.94 (br d, 1 H), 3.70 (br t, 1 H), 3.51-3.35 (m, 2 H), 2.40 (q, 1 H), 2.31-2.22 (m, 1 H), 2.20- 2.05 (m, 2 H), 2.00 (br d, 2 H), 1.895-1.80 (m, 3 H), 1.75-1.59 (m, 3 H). | m/z 513.2 [M + H]⁺; (column: DAICEL CHIRALPAK IC(250 mm * 30 mm, 10 um); mobile phase: [0.1% NH₃H₂O EtOH]; B%: 55%-55%, min) Rt = 1.285 min 90.0% ee |
| 104 | 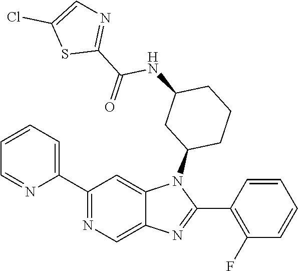 [SMILES: O=C(N[C@H]1CCC[C@@H](n2c(-c3ccccc3F)nc3cnc(-c4ccccn4)cc32)C1)c1ncc(Cl)s1] | (400 MHz, CD₃OD) δ 8.97 (s, 1 H), 8.68 (s, 1 H), 8.60 (br s, 1 H), 8.38 (br s, 1 H), 7.90-7.86 (m, 1 H), 7.68 (s, 1 H), 7.66-7.57 (m, 2 H), 7.41-7.30 (m, 3 H), 4.21 (br s, 1 H), 3.95-3.81 (m, 1 H), 2.58-2.42 (m, 1 H), 2.38- 2.25 (m, 1 H), 2.21-2.11 (m, 1 H), 2.00-1.86 (m, 3 H), 1.66-1.52 (m, 1 H), 1.47- 1.32 (m, 1 H). | m/z 533.2 [M + H]⁺ |
| 105 | 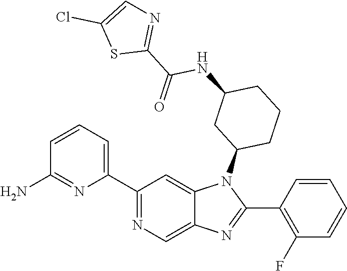 [SMILES: Nc1cccc(-c2cc3c(cn2)nc(-c2ccccc2F)n3[C@@H]2CCC[C@H](NC(=O)c3ncc(Cl)s3)C2)n1] | (400 MHz, CDCl₃) δ 9.17 (d, 1 H), 8.61 (s, 1 H), 7.90 (d, 1 H), 7.70-7.58 (m, 4 H), 7.38 (td, 1 H), 7.30 (d, 1 H), 7.21 (br d, 1 H), 6.57 (d, 1 H), 4.73 (br s, 2 H), 4.23 (br t, 1 H), 4.08-3.95 (m, 1 H), 2.45-2.30 (m, 3 H), 2.13- 1.96 (m, 3 H), 1.56-1.37 (m, 2 H). | m/z 548.2 [M + H]⁺; |
| 116 | 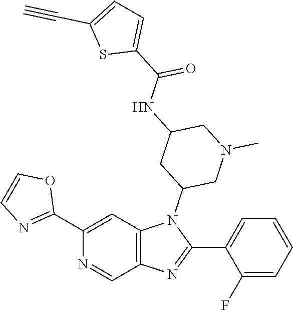 [SMILES: C#Cc1ccc(C(=O)NC2CC(n3c(-c4ccccc4F)nc4cnc(-c5ncco5)cc43)CN(C)C2)s1] | (400 MHz, CD₃OD) δ 9.11 (s, 1 H), 8.67 (s, 1 H), 8.17 (s, 1 H), 7.78-7.75 (m, 2 H), 7.60-7.47 (m, 4 H), 7.24 (d, 1 H), 4.57-4.51 (m, 1 H), 4.22-4.17 (m, 1 H), 3.14- 3.11 (m, 2 H), 2.97-2.91 (m, 1 H), 2.52-2.38 (m, 5 H), 2.27-2.21 (m, 1 H). | m/z 527.1 [M + H]⁺ |
| | (relative stereochemistry at | | |
| | centers 3 and 5 of piperidine | | |
| | ring is cis) | | |
| 117 | 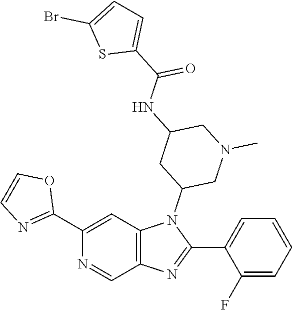 [SMILES: CN1CC(NC(=O)c2ccc(Br)s2)CC(n2c(-c3ccccc3F)nc3cnc(-c4ncco4)cc32)C1] | (400 MHz, CD₃OD) δ 9.11 (s, 1 H), 8.67 (s, 1 H), 8.17 (s, 1 H), 7.78-7.63 (m, 2 H), 7.51-7.45 (m, 4 H), 7.15 (d, 1 H), 4.57-4.51 (m, 1 H), 4.20-4.15 (m, 1 H), 3.14- 3.11 (m, 2 H), 2.96-2.91 (m, 1 H), 2.53-2.37 (m, 5 H), 2.26-2.21 (m, 1 H). | m/z 581.0 [M + H]⁺ |
| | (relative stereochemistry at | | |
| | centers 3 and 5 of piperidine | | |
| | ring is cis) | | |
| 118 | 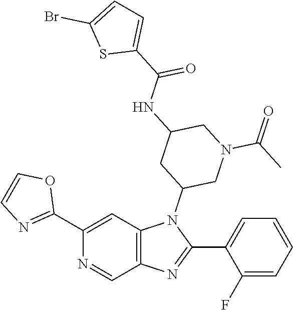 [SMILES: CC(=O)N1CC(NC(=O)c2ccc(Br)s2)CC(n2c(-c3ccccc3F)nc3cnc(-c4ncco4)cc32)C1] | (400 MHz, CD₃OD) δ 9.13 (s, 1 H), 8.73 (s, 1 H), 8.18 (s, 1 H), 7.79-7.74 (m, 2 H), 7.54-7.44 (m, 4 H), 7.17 (d, 1 H), 4.50-4.22 (m, 2 H), 4.10-4.03 (m, 1 H), 3.54- 3.50 (m, 1 H), 3.30-3.23 (m, 1 H), 2.83-2.73 (m, 1 H), 2.21 (s, 3 H). | m/z 611.3 [M + H]⁺ |
| | (relative stereochemistry at | | |
| | centers 3 and 5 of piperidine | | |
| | ring is cis) | | |
| 119 | 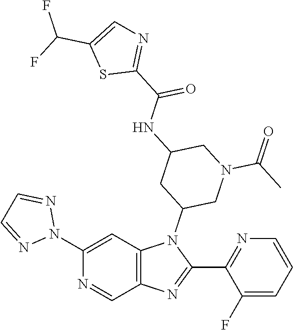 [SMILES: CC(=O)N1CC(NC(=O)c2ncc(C(F)F)s2)CC(n2c(-c3ncccc3F)nc3cnc(-n4nccn4)cc32)C1] | (400 MHz, CD₃OD) δ 9.01 (s, 1 H), 8.73-8.67 (m, 1 H), 8.62 (d, 1 H), 8.19-8.14 (m, 1 H), 8.10 (d, 2 H), 7.99- 7.91 (m, 1 H), 7.79-7.72 (m, 1 H), 7.33-7.03 (m, 1 H), 5.09 (br s, 1 H), 4.53 (br d, 1 H), 4.25-3.95 (m, 2 H), 3.48 (br t, 1 H), 3.00-2.81 (m, 2 H), 2.60-2.41 (m, 1 H), 2.29-2.20 (m, 3 H). | m/z 583.4 [M + H]⁺ |
| | (relative stereochemistry at | | |
| | centers 3 and 5 of piperidine | | |
| | ring is cis) | | |
| 120 | 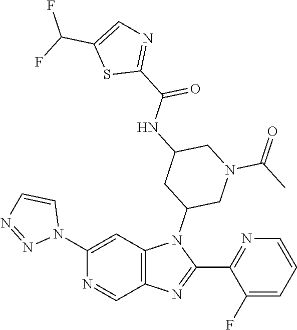 [SMILES: CC(=O)N1CC(NC(=O)c2ncc(C(F)F)s2)CC(n2c(-c3ncccc3F)nc3cnc(-n4ccnn4)cc32)C1] | (400 MHz, CD₃OD) δ 9.04 (s, 1 H), 8.89 (s, 1 H), 8.72- 8.67 (m, 2 H), 8.17 (br d, 1 H), 8.02-7.92 (m, 2 H), 7.81-7.71 (m, 1 H), 7.35- 7.01 (m, 1 H), 5.09 (br s, 1 H), 4.53 (br d, 1 H), 4.24- 3.95 (m, 2 H), 3.49 (br t, 1 H), 3.00-2.84 (m, 2 H), 2.60-2.41 (m, 1 H), 2.30- 2.20 (m, 3 H). | m/z 583.3 [M + H]⁺ |
| | (relative stereochemistry at | | |
| | centers 3 and 5 of piperidine | | |
| | ring is cis) | | |
| 121 | 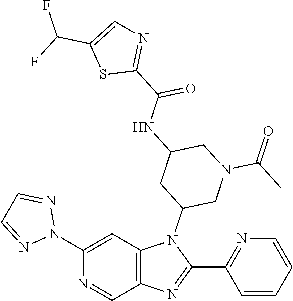 [SMILES: CC(=O)N1CC(NC(=O)c2ncc(C(F)F)s2)CC(n2c(-c3ccccn3)nc3cnc(-n4nccn4)cc32)C1] | (400 MHz, CD₃OD) δ 8.96 (s, 1 H), 8.85-8.77 (m, 1 H), 8.56 (d, 1 H), 8.28 (dd, 1 H), 8.16 (br d, 1 H), 8.12- 8.00 (m, 3 H), 7.66-7.56 (m, 1 H), 7.34-7.00 (m, 1 H), 5.86-5.61 (m, 1 H), 4.96 (br dd, 0.5 H), 4.56 (br d, 0.5 H), 4.28-3.96 (m, 2 H), 3.61-3.34 (m, 1 H), 3.06- 2.81 (m, 2 H), 2.61-2.38 (m, 1 H), 2.34-2.21 (m, 3 H). | m/z 565.3 [M + H]⁺ |
| | (relative stereochemistry at | | |
| | centers 3 and 5 of piperidine | | |
| | ring is cis) | | |
| 122 | 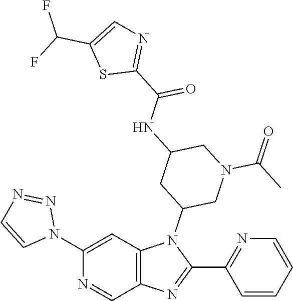 [SMILES: CC(=O)N1CC(NC(=O)c2ncc(C(F)F)s2)CC(n2c(-c3ccccn3)nc3cnc(-n4ccnn4)cc32)C1] | (400 MHz, DMSO-d₆) δ 9.40-9.28 (m, 1 H), 9.10 (s, 1 H), 8.92 (s, 1 H), 8.87- 8.77 (m, 1 H), 8.65-8.56 (m, 1 H), 8.34 (br s, 1 H), 8.32- 8.25 (m, 1 H), 8.17-8.08 (m, 1 H), 8.04 (s, 1 H), 7.72- 7.62 (m, 1 H), 7.61-7.30 (m, 1 H), 5.75-5.55 (m, 1 H), 4.79-4.31 (m, 2 H), 4.22- 3.76 (m, 2 H), 3.06-2.78 (m, 2 H), 2.34-2.23 (m, 1 H), 2.20-2.08 (m, 3 H). | m/z 565.3 [M + H]⁺ |
| | (relative stereochemistry at | | |
| | centers 3 and 5 of piperidine | | |
| | ring is cis) | | |
| 123 | 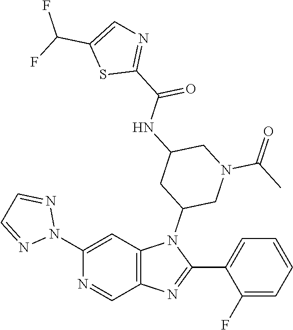 [SMILES: CC(=O)N1CC(NC(=O)c2ncc(C(F)F)s2)CC(n2c(-c3ccccc3F)nc3cnc(-n4nccn4)cc32)C1] | (400 MHz, CD₃OD) δ 8.97 (s, 1 H), 8.58 (s, 1 H), 8.18- 8.13 (m, 1 H), 8.09 (s, 2 H), 7.80-7.69 (m, 2 H), 7.54- 7.38 (m, 2 H), 7.33-7.02 (m, 1 H), 4.75-4.34 (m, 2 H), 4.30-4.05 (m, 2 H), 3.51- 3.32 (m, 1 H), 3.00-2.75 (m, 2 H), 2.45 (br d, 1 H), 2.18- 2.05 (m, 3 H). | m/z 582.3 [M + H]⁺ |
| | (relative stereochemistry at | | |
| | centers 3 and 5 of piperidine | | |
| | ring is cis) | | |
| 124 | 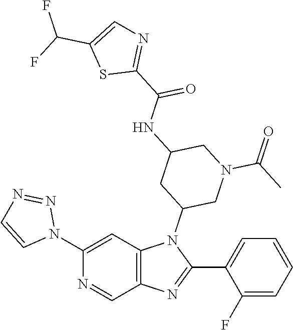 [SMILES: CC(=O)N1CC(NC(=O)c2ncc(C(F)F)s2)CC(n2c(-c3ccccc3F)nc3cnc(-n4ccnn4)cc32)C1] | (400 MHz, CD₃OD) δ 9.01- 8.96 (m, 1 H), 8.87 (s, 1 H), 8.67-8.61 (m, 1 H), 8.19- 8.11 (m, 1 H), 7.96 (s, 1 H), 7.79-7.69 (m, 2 H), 7.51- 7.39 (m, 2 H), 7.32-7.02 (m, 1 H), 4.72-4.32 (m, 2 H), 4.24-4.03 (m, 2 H), 3.51- 3.32 (m, 1.5 H), 2.98-2.79 (m, 1.5 H), 2.50-2.38 (m, 1 H), 2.18-2.06 (m, 3 H). | m/z 582.4 [M + H]⁺ |
| | (relative stereochemistry at | | |
| | centers 3 and 5 of piperidine | | |
| | ring is cis) | | |
| 126 | 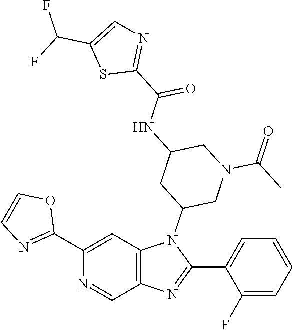 [SMILES: CC(=O)N1CC(NC(=O)c2ncc(C(F)F)s2)CC(n2c(-c3ccccc3F)nc3cnc(-c4ncco4)cc32)C1] | (400 MHz, DMSO-d₆) δ 9.36-9.19 (m, 1 H), 9.12 (br s, 1 H), 8.58 (br s, 1 H), 8.35-8.26 (m, 2 H), 7.78- 7.63 (m, 2 H), 7.58-7.26 (m, 4 H), 4.62-4.25 (m, 2 H), 4.12-3.69 (m, 2 H), 3.25- 3.10 (m, 1 H), 2.90-2.61 (m, 2 H), 2.30-2.11 (m, 1 H), 1.95-1.86 (m, 3 H). | m/z 582.2 [M + H]⁺ |
| | (relative stereochemistry at | | |
| | centers 3 and 5 of piperidine | | |
| | ring is cis) | | |
| 127 | 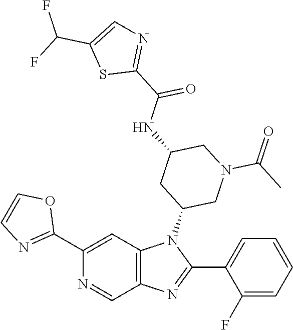 [SMILES: CC(=O)N1C[C@@H](NC(=O)c2ncc(C(F)F)s2)C[C@@H](n2c(-c3ccccc3F)nc3cnc(-c4ncco4)cc32)C1] | (400 MHz, CDCl₃) δ 9.27 (s, 1 H), 8.56-8.49 (m, 1 H), 8.09-7.98 (m, 1 H), 7.91 (s, 1 H), 7.78-7.31 (m, 6 H), 7.12-6.75 (m, 1 H), 5.10- 4.87 (m, 1 H), 4.43-3.96 (m, 3 H), 4.43-3.19 (m, 1 H), 2.61-2.47 (m, 3 H) 2.22- 2.13 (m, 3 H). | m/z 582.2 [M + H]⁺; (column: DAICEL CHIRALPAK AS(250 mm * 30 mm, 10 um); mobile phase: [0.1% NH₃H₂O EtOH]; B%: 25%-25%, min); Rt = 2.444 min 100% ee |
| 128 | 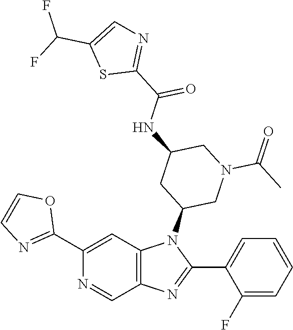 [SMILES: CC(=O)N1C[C@H](NC(=O)c2ncc(C(F)F)s2)C[C@H](n2c(-c3ccccc3F)nc3cnc(-c4ncco4)cc32)C1] | (400 MHz, CDCl₃) δ 9.27 (s, 1 H), 8.55 (br d, 1 H), 8.08-7.99 (m, 1 H), 7.91 (s, 1 H), 7.79-7.30 (m, 6 H), 7.12-6.75 (m, 1 H), 5.11- 4.88 (m, 1 H), 4.41-3.92 (m, 3 H), 3.44-3.16 (m, 1 H), 2.85-2.40 (m, 3 H), 2.23- 2.12 (m, 3 H). | m/z 582.2 [M + H]⁺; (column: DAICEL CHIRALPAK AS(250 mm * 30 mm, 10 um); mobile phase: [0.1% NH₃H₂O EtOH]; B%: 25%-25%, min); Rt = 2.862 min 99.2% ee |
| 130 | 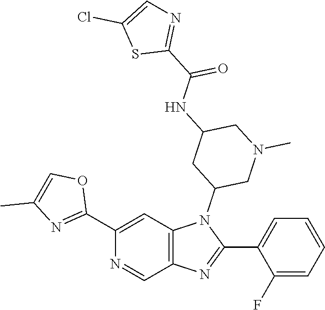 [SMILES: Cc1coc(-c2cc3c(cn2)nc(-c2ccccc2F)n3C2CC(NC(=O)c3ncc(Cl)s3)CN(C)C2)n1] | (400 MHz, CD₃OD) δ 9.09 (s, 1 H), 8.67 (s, 1 H), 7.88 (s, 1 H), 7.83 (s, 1 H), 7.77- 7.76 (m, 2 H), 7.54-7.42 (m, 2 H), 4.53 (br s, 1 H), 4.21 (br s, 1 H), 3.12-3.07 (m, 2H), 2.96 (t, 1 H), 2.63-2.55 (m, 1 H), 2.44 (s, 3 H), 2.35-2.31 (m, 5 H). | m/z 552.2 [M + H]⁺ |
| | (relative stereochemistry at | | |
| | centers 3 and 5 of piperidine | | |
| | ring is cis) | | |
| 158 | 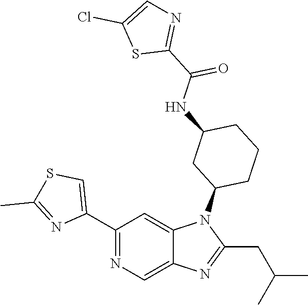 [SMILES: Cc1nc(-c2cc3c(cn2)nc(CC(C)C)n3[C@@H]2CCC[C@H](NC(=O)c3ncc(Cl)s3)C2)cs1] | (400 MHz, DMSO-d₆) δ 8.87-8.78 (m, 2 H), 8.35 (s, 1 H), 8.09 (s, 1 H), 8.07 (s, 1 H), 4.69-4.55 (m, 1 H), 4.25-4.12 (m, 1 H), 2.86 (d, 2 H), 2.68 (s, 3 H), 2.63-2.52 (m, 1 H), 2.27-2.09 (m, 2 H), 2.01-1.75 (m, 4 H), 1.74- 1.59 (m, 2 H), 1.05-0.96 (m, 6 H). | m/z 515.6 [M + H]⁺ |
| 160 | 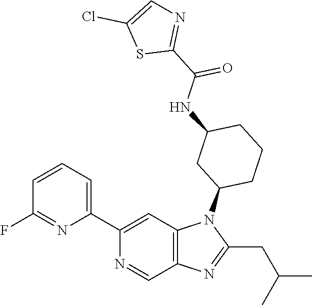 [SMILES: CC(C)Cc1nc2cnc(-c3cccc(F)n3)cc2n1[C@@H]1CCC[C@H](NC(=O)c2ncc(Cl)s2)C1] | (400 MHz, DMSO-d₆) δ 9.16 (d, 1 H), 8.96 (s, 1 H), 8.49 (s, 1 H), 8.41-8.34 (m, 1 H), 8.17-8.08 (m, 1 H), 8.05 (s, 1 H), 7.23-7.16 (m, 1 H), 4.74-4.61 (m, 1 H), 4.28-4.13 (m, 1 H), 2.91 (d, 2 H), 2.58-2.52 (m, 1 H), 2.29-2.06 (m, 2 H), 2.05- 1.85 (m, 4 H), 1.75-1.56 (m, 2 H), 1.02 (d, 6 H). | m/z 513.1 [M + H]⁺ |
| 161 | 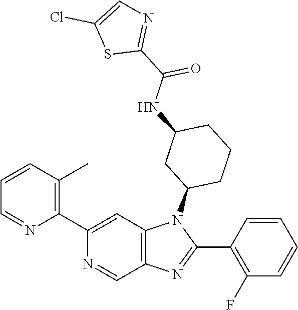 [SMILES: Cc1cccnc1-c1cc2c(cn1)nc(-c1ccccc1F)n2[C@@H]1CCC[C@H](NC(=O)c2ncc(Cl)s2)C1] | (400 MHz, CD₃OD) δ 9.06 (s, 1 H), 8.54-8.49 (m, 1 H), 8.16 (s, 1 H), 7.86 (d, 1 H), 7.79-7.70 (m, 3 H), 7.51- 7.41 (m, 3 H), 4.35-4.26 (m, 1 H), 4.00-3.88 (m, 1 H), 2.60-2.48 (m, 1 H), 2.44 (s, 3 H), 2.38-2.22 (m, 2 H), 2.10-1.90 (m, 3 H), 1.63- 1.39 (m, 2 H). | m/z 547.2 [M + H]⁺ |
| 163 | 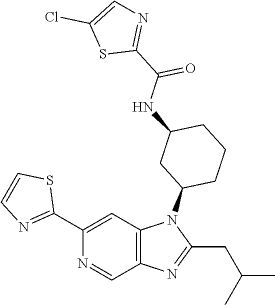 [SMILES: CC(C)Cc1nc2cnc(-c3nccs3)cc2n1[C@@H]1CCC[C@H](NC(=O)c2ncc(Cl)s2)C1] | (400 MHz, CDCl₃) δ 9.03 (s, 1 H), 8.34 (s, 1 H), 7.94- 7.93 (m, 1 H), 7.66 (s, 1 H), 7.45-7.44 (m, 1 H), 7.20- 7.18 (m, 1 H), 4.44-4.37 (m, 1 H), 4.23-4.18 (m, 1 H), 2.86-2.84 (m, 2 H), 2.38- 2.20 (m, 5 H), 2.15-2.12 (m, 1 H), 2.03-1.99 (m, 1 H), 1.66-1.55 (m, 2 H), 1.12- 1.10 (m, 6 H) | m/z 501.1 [M + H]⁺ |
| 165 | 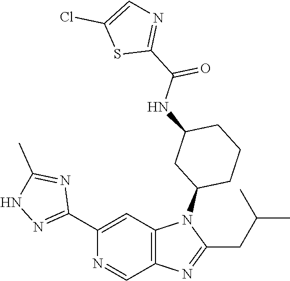 [SMILES: Cc1nc(-c2cc3c(cn2)nc(CC(C)C)n3[C@@H]2CCC[C@H](NC(=O)c3ncc(Cl)s3)C2)n[nH]1] | (400 MHz, DMSO-d₆) δ 14.18 (br s, 1 H), 9.18-9.16 (m, 1 H), 8.95 (br s, 1 H), 8.29 (br s, 1 H), 8.06 (s, 1 H), 4.66 (br s, 1 H), 4.20 (br s, 1 H), 2.92-2.90 (m, 2 H), 2.35 (br s, 4 H), 2.24 (br s, 1 H), 1.99 (br s, 3 H), 1.87 (br s, 2 H), 1.65 (br s, 2 H), 1.02-1.00 (m, 6 H) | m/z 499.2 [M + H]⁺ |
| 168 | 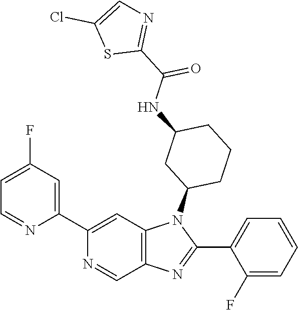 [SMILES: O=C(N[C@H]1CCC[C@@H](n2c(-c3ccccc3F)nc3cnc(-c4cc(F)ccn4)cc32)C1)c1ncc(Cl)s1] | (400 MHz, DMSO-d₆) δ 9.18-9.10 (m, 2 H), 8.85- 8.78 (m, 2 H), 8.30-8.22 (m, 1 H), 8.04 (s, 1 H), 7.78-7.69 (m, 2 H), 7.56-7.39 (m, 3 H), 4.32-4.18 (m, 1 H), 4.02- 3.87 (m, 1 H), 2.69-2.55 (m, 1 H), 2.14-1.81 (m, 5 H), 1.66-1.41 (m, 2 H). | m/z 551.2 [M + H]⁺ |
| 169 | 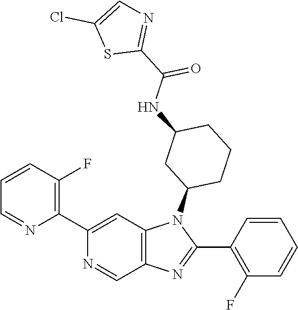 [SMILES: O=C(N[C@H]1CCC[C@@H](n2c(-c3ccccc3F)nc3cnc(-c4ncccc4F)cc32)C1)c1ncc(Cl)s1] | (400 MHz, DMSO-d₆) δ 9.13 (s, 1 H), 8.97 (d, 1 H), 8.65-8.61 (m, 1 H), 8.35 (s, 1 H), 8.03 (s, 1 H), 7.93-7.86 (m, 1 H), 7.74 (t, 2 H), 7.62- 7.45 (m, 3 H), 4.30-4.18 (m, 1 H), 3.99-3.87 (m, 1 H), 2.65-2.54 (m, 1 H), 2.16- 2.00 (m, 2 H), 1.98-1.74 (m, 3 H), 1.60-1.37 (m, 2 H). | m/z 551.4 [M + H]⁺ |
| 170 | 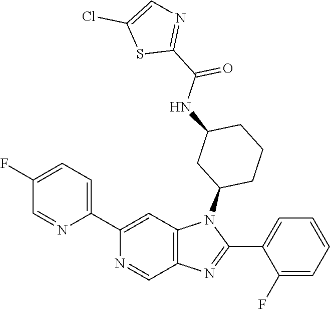 [SMILES: O=C(N[C@H]1CCC[C@@H](n2c(-c3ccccc3F)nc3cnc(-c4ccc(F)cn4)cc32)C1)c1ncc(Cl)s1] | (400 MHz, DMSO-d₆) δ 9.18-9.10 (m, 2 H), 8.75 (d, 1 H), 8.71 (s, 1 H), 8.58-8.53 (m, 1 H), 8.04 (s, 1 H), 7.94- 7.87 (m, 1 H), 7.77-7.71 (m, 2 H), 7.56-7.45 (m, 2 H), 4.31-4.16 (m, 1 H), 4.00- 3.88 (m, 1 H), 2.70-2.55 (m, 1 H), 2.20-1.77 (m, 5 H), 1.66-1.36 (m, 2 H). | m/z 551.2 [M + H]⁺ |
| 171 | 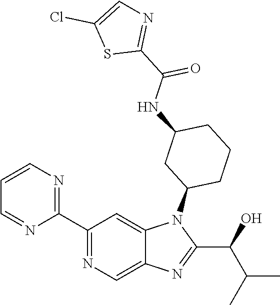 [SMILES: CC(C)[C@H](O)c1nc2cnc(-c3ncccn3)cc2n1[C@@H]1CCC[C@H](NC(=O)c2ncc(Cl)s2)C1] | (400 MHz, CDCl₃) δ 7.55 (br s, 1 H), 7.45-7.14 (m, 5 H), 4.76-4.67 (m, 2 H), 4.16-4.11 (m, 1 H), 2.67- 2.12 (m, 4 H), 1.99-1.97 (m, 1 H), 1.63-1.45 (m, 4 H), 1.10-1.06 (m, 3 H), 0.90- 0.84 (m, 3 H). | m/z 512.2 [M + H]⁺ |
| 172 | 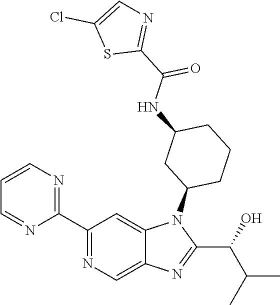 [SMILES: CC(C)[C@@H](O)c1nc2cnc(-c3ncccn3)cc2n1[C@@H]1CCC[C@H](NC(=O)c2ncc(Cl)s2)C1] | (400 MHz, CDCl₃) δ 9.00- 8.93 (m, 3 H), 7.64 (s, 1 H), 7.19-7.02 (m, 2 H), 4.85- 4.65 (m, 2 H), 4.23-4.20 (m, 1 H), 2.44-2.11 (m, 5 H), 1.70-1.64 (m, 2 H), 1.45- 1.25 (m, 2 H), 1.02-0.93 (m, 6 H). | m/z 512.2 [M + H]⁺ |
| 173 | 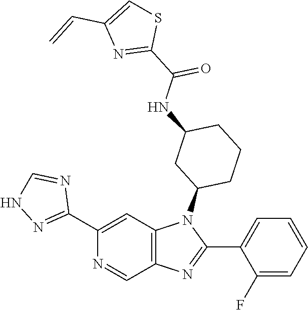 [SMILES: C=Cc1csc(C(=O)N[C@H]2CCC[C@@H](n3c(-c4ccccc4F)nc4cnc(-c5nc[nH]n5)cc43)C2)n1] | (400 MHz, DMSO-d₆) δ 9.16 (s, 1 H), 8.94 (d, 1 H), 8.52 (s, 1 H), 8.35-8.19 (m, 1 H), 7.90 (s, 1 H), 7.79-7.70 (m, 2 H), 7.59-7.45 (m, 2 H), 6.85-6.74 (m, 1 H), 6.15- 6.06 (m, 1 H), 5.45-5.38 (m, 1 H), 4.29-4.23 (m, 1 H), 3.99-3.97 (m, 1 H), 2.65- 2.55 (m, 1 H), 2.17-2.03 (m, 2 H), 2.01-1.86 (m, 2 H), 1.85-1.78 (m, 1 H), 1.70- 1.58 (m, 1 H), 1.51-1.39 (m, 1 H). | m/z 515.2 [M + H]⁺ |
| 175 | 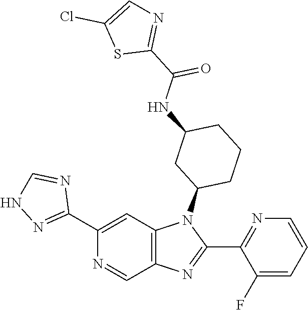 [SMILES: O=C(N[C@H]1CCC[C@@H](n2c(-c3ncccc3F)nc3cnc(-c4nc[nH]n4)cc32)C1)c1ncc(Cl)s1] | (400 MHz, DMSO-d₆) δ 14.73 (s, 1 H), 9.18 (s, 1 H), 9.11 (d, 1 H), 8.73-8.67 (m, 1 H), 8.54 (s, 1 H), 8.18-8.00 (m, 3 H), 7.84-7.75 (m, 1 H), 4.81-4.68 (m, 1 H), 4.05- 3.90 (m, 1 H), 2.65-2.53 (m, 1 H), 2.18-1.96 (m, 3 H), 1.94-1.77 (m, 2 H), 1.68- 1.37 (m, 2 H). | m/z 524.6 [M + H]⁺ |
| 176 | 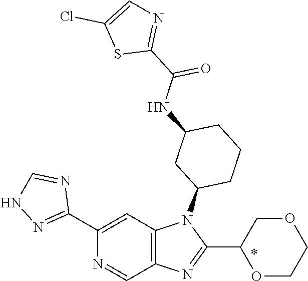 [SMILES: O=C(N[C@H]1CCC[C@@H](n2c(C3COCCO3)nc3cnc(-c4nc[nH]n4)cc32)C1)c1ncc(Cl)s1] | (400 MHz, CDCl₃) δ 9.02 (s, 1 H), 8.35 (s, 1 H), 8.04 (s, 1 H), 7.57 (s, 1 H), 7.24 (d, 1 H), 4.91-4.85 (m, 1 H), 4.68-4.62 (m, 1 H), 4.25- 4.17 (m, 2 H), 3.91-3.65 (m, 5 H), 2.37-2.29 (m, 2 H), 2.18-2.11 (m, 2 H), 2.08- 2.02 (m, 2 H), 1.61-1.55 (m, 2 H). | m/z 515.1 [M + H]⁺; (column: DAICEL CHIRALPAK IG (100 × 4.6 mm, 5 um); mobile phase: [0.05% diethylamine IPA]; B%: 40%-40%, min) Rt = 3.449 min 95.8% ee |
| 177 | 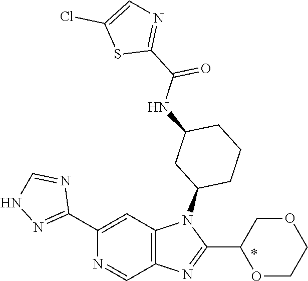 [SMILES: O=C(N[C@H]1CCC[C@@H](n2c(C3COCCO3)nc3cnc(-c4nc[nH]n4)cc32)C1)c1ncc(Cl)s1] | (400 MHz, CDCl₃) δ 9.02 (s, 1 H), 8.35 (s, 1 H), 8.04 (s, 1 H), 7.57 (s, 1 H), 7.24- 7.20 (m, 1 H), 4.90-4.80 (m, 1 H), 4.67-4.59 (m, 1 H), 4.22-4.19 (m, 1 H), 4.15- 4.07 (m, 1 H), 3.94-3.85 (m, 2 H), 3.83-3.78 (m, 1 H), 3.78-3.71 (m, 1 H), 3.69- 3.59 (m, 1 H), 2.44-2.35 (m, 1 H), 2.29-2.19 (m, 2 H), 2.18-2.12 (m, 1 H), 2.07- 2.02 (m, 1 H), 1.97-1.93 (m, 1 H), 1.53-1.47 (m, 2 H). | m/z 515.1 [M + H]⁺; (column: DAICEL CHIRALPAK IG (100 × 4.6 mm, 5 um); mobile phase: [0.05% diethylamine IPA]; B%: 40%-40%, min) Rt = 2.915 min 95.1% ee |
| 178 | 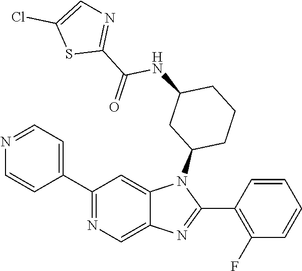 [SMILES: O=C(N[C@H]1CCC[C@@H](n2c(-c3ccccc3F)nc3cnc(-c4ccncc4)cc32)C1)c1ncc(Cl)s1] | (400 MHz, DMSO-d₆) δ 9.16 (s, 1 H), 8.85 (d, 1 H), 8.78-8.70 (m, 2 H), 8.40 (s, 1 H), 8.23-8.17 (m, 2 H), 8.06 (s, 1 H), 7.78-7.69 (m, 2 H), 7.57-7.44 (m, 2 H), 4.33-4.22 (m, 1 H), 4.02- 3.89 (m, 1 H), 2.68-2.59 (m, 1 H), 2.31-2.14 (m, 1 H), 2.07-2.00 (m, 1 H), 1.94- 1.78 (m, 3 H), 1.71-1.56 (m, 1 H), 1.52-1.35 (m, 1 H). | m/z 533.1 [M + H]⁺ |
| 179 | 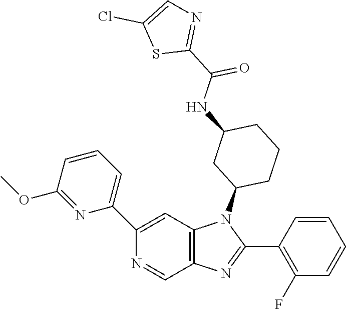 [SMILES: COc1cccc(-c2cc3c(cn2)nc(-c2ccccc2F)n3[C@@H]2CCC[C@H](NC(=O)c3ncc(Cl)s3)C2)n1] | (400 MHz, DMSO-d₆) δ 9.19-9.06 (m, 2 H), 8.83 (s, 1 H), 8.11-8.00 (m, 2 H), 7.88 (t, 1 H), 7.81-7.70 (m, 2 H), 7.57-7.44 (m, 2 H), 6.89 (d, 1 H), 4.33-4.21 (m, 1 H), 4.13 (s, 3 H), 3.99-3.86 (m, 1 H), 2.70-2.58 (m, 1 H), 2.28-2.13 (m, 1 H), 2.12- 1.85 (m, 4 H), 1.56-1.40 (m, 2 H). | m/z 563.2 [M + H]⁺ |
| 180 | 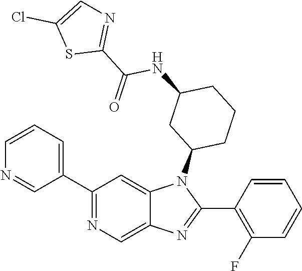 [SMILES: O=C(N[C@H]1CCC[C@@H](n2c(-c3ccccc3F)nc3cnc(-c4cccnc4)cc32)C1)c1ncc(Cl)s1] | (400 MHz, DMSO-d₆) δ 9.41 (d, 1 H), 9.14 (s, 1 H), 8.86 (d, 1 H), 8.69-8.63 (m, 1 H), 8.61-8.53 (m, 1 H), 8.34 (s, 1 H), 8.05 (s, 1 H), 7.79-7.68 (m, 2 H), 7.63- 7.44 (m, 3 H), 4.33-4.18 (m, 1 H), 4.04-3.89 (m, 1 H), 2.73-2.58 (m, 1 H), 2.36- 2.19 (m, 1 H), 2.10-1.98 (m, 1 H), 1.96-1.75 (m, 3 H), 1.71-1.57 (m, 1 H), 1.49- 1.39 (m, 1 H). | m/z 533.1 [M + H]⁺ |
| 181 | 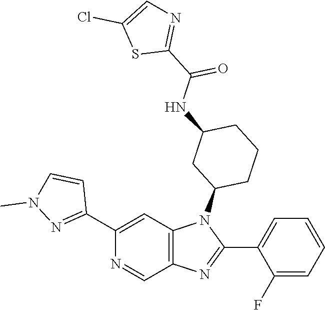 [SMILES: Cn1ccc(-c2cc3c(cn2)nc(-c2ccccc2F)n3[C@@H]2CCC[C@H](NC(=O)c3ncc(Cl)s3)C2)n1] | (400 MHz, DMSO-d₆) δ 9.13 (d, 1 H), 9.00 (s, 1 H), 8.22 (s, 1 H), 8.04 (s, 1 H), 7.79 (d, 1 H), 7.75-7.67 (m, 2 H), 7.53-7.42 (m, 2 H), 6.86 (d, 1 H), 4.28-4.07 (m, 1 H), 3.97 (s, 3 H), 3.96-3.87 (m, 1 H), 2.61-2.52 (m, 1 H), 2.15-1.97 (m, 2 H), 1.97- 1.85 (m, 2 H), 1.84-1.70 (m, 1 H), 1.66-1.50 (m, 1 H), 1.49-1.33 (m, 1 H). | m/z 536.1 [M + H]⁺ |
| 182 | 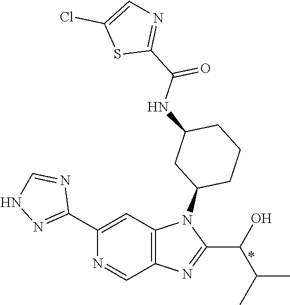 [SMILES: CC(C)C(O)c1nc2cnc(-c3nc[nH]n3)cc2n1[C@@H]1CCC[C@H](NC(=O)c2ncc(Cl)s2)C1] | (400 MHz, CDCl₃) δ 9.14 (s, 1 H), 8.39 (s, 1 H), 8.12 (s, 1 H), 7.67 (s, 1 H), 7.23- 7.20 (m, 1 H), 4.76-4.69 (m, 2 H), 4.19 (br s, 1 H), 3.48 (br s, 1 H), 2.41- 2.26 (m, 5 H), 2.24-2.03 (m, 2 H), 1.66-1.61 (m, 2 H), 1.11-1.01 (m, 6 H). | m/z 501.2 [M + H]⁺; (column: DAICEL CHIRALPAK AD (250 mm * 30 mm, 10 um); mobile phase: [0.1% NH₃H₂O IPA]; B%: 35%- 35%, min) Rt = 2.084 min 99.0% ee |
| 183 | 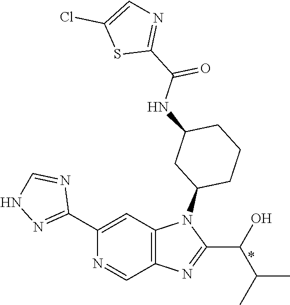 [SMILES: CC(C)C(O)c1nc2cnc(-c3nc[nH]n3)cc2n1[C@@H]1CCC[C@H](NC(=O)c2ncc(Cl)s2)C1] | (400 MHz, CDCl₃) δ 9.12 (s, 1 H), 8.37 (s, 1 H), 8.12 (s, 1 H), 7.66 (s, 1 H), 7.25- 7.20 (m, 1 H), 4.80-4.69 (m, 2 H), 4.20 (br s, 1 H), 3.42 (br s, 1 H), 2.41-2.26 (m, 5 H), 2.24-2.22 (m, 2 H), 1.65-1.61 (m, 2 H), 1.11-1.04 (m, 6 H). | m/z 501.2 [M + H]⁺; (column: DAICEL CHIRALPAK AD (250 mm * 30 mm, 10 um); mobile phase: [0.1% NH₃H₂O IPA]; B%: 35%- 35%, min) Rt = 1.616 min 100% ee |
| 184 | 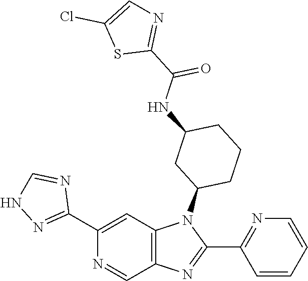 [SMILES: O=C(N[C@H]1CCC[C@@H](n2c(-c3ccccn3)nc3cnc(-c4nc[nH]n4)cc32)C1)c1ncc(Cl)s1] | (400 MHz, DMSO-d₆) δ 14.67 (s, 1 H), 9.25-9.10 (m, 2 H), 8.87-8.78 (m, 1 H), 8.50 (s, 1 H), 8.27-8.22 (m, 1 H), 8.13-8.07 (m, 1 H), 8.05 (s, 1 H), 7.67-7.60 (m, 1 H), 5.67-5.57 (m, 1 H), 4.08-3.94 (m, 1 H), 2.71- 2.59 (m, 1 H), 2.20-1.83 (m, 5 H), 1.75-1.59 (m, 1 H), 1.57-1.43 (m, 1 H). | m/z 506.1 [M + H]⁺ |
| 185 | 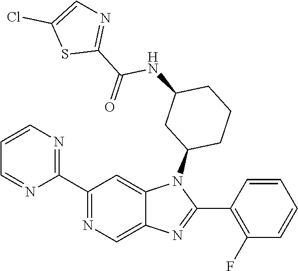 [SMILES: O=C(N[C@H]1CCC[C@@H](n2c(-c3ccccc3F)nc3cnc(-c4ncccn4)cc32)C1)c1ncc(Cl)s1] | (400 MHz, CD₃OD) δ 9.01 (br s, 4 H), 7.79-7.71 (m, 3 H), 7.51-7.45 (m, 3 H), 4.34-4.31 (m, 1 H), 4.02- 3.96 (m, 1 H), 2.63-2.54 (m, 1 H), 2.38-2.29 (m, 2 H), 2.10-2.02 (m, 3 H), 1.69- 1.66 (m, 1 H), 1.53-1.49 (m, 1 H) | m/z 534.2 [M + H]⁺ |
| 186 | 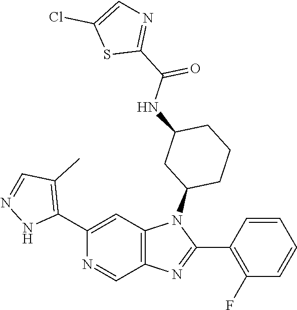 [SMILES: Cc1cn[nH]c1-c1cc2c(cn1)nc(-c1ccccc1F)n2[C@@H]1CCC[C@H](NC(=O)c2ncc(Cl)s2)C1] | (400 MHz, CDCl₃) δ 9.17 (s, 1 H), 7.95 (s, 1 H), 7.69- 7.53(m, 3 H), 7.53 (s, 1 H), 7.42 (br s, 1 H), 7.31-7.29 (m, 2 H), 7.28-7.24 (m, 1 H), 4.26 (br s, 1 H), 4.03 (br s, 1 H), 2.49 (s, 3 H), 2.32- 2.26 (m, 3 H), 2.19-2.06 (m, 3 H), 1.55-1.47 (m, 1 H). | m/z 536.2 [M + H]⁺ |
| 187 | 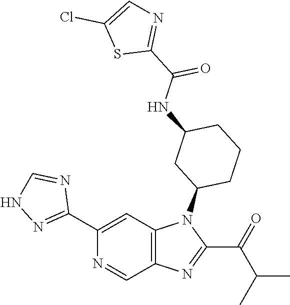 [SMILES: CC(C)C(=O)c1nc2cnc(-c3nc[nH]n3)cc2n1[C@@H]1CCC[C@H](NC(=O)c2ncc(Cl)s2)C1] | (400 MHz, CDCl₃) δ 9.16 (s, 1 H), 8.38 (s, 1 H), 8.06 (s, 1 H), 7.57 (s, 1 H), 7.11- 7.09 (m, 1 H), 5.48-5.43 (m, 1 H), 4.15-4.01 (m, 2 H), 2.35-2.14 (m, 4 H), 2.03- 2.00 (m, 2 H), 1.65-1.60 (m, 2 H), 1.24-1.22 (m, 6 H). | m/z 499.1 [M + H]⁺ |
| 188 | 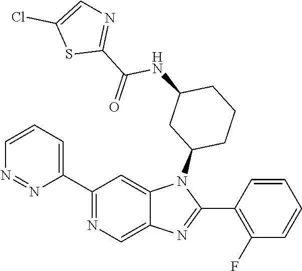 [SMILES: O=C(N[C@H]1CCC[C@@H](n2c(-c3ccccc3F)nc3cnc(-c4cccnn4)cc32)C1)c1ncc(Cl)s1] | (400 MHz, CDCl₃) δ 9.95 (s, 1 H), 9.38-9.37 (m, 1 H), 9.26 (s, 1 H), 8.25-8.24 (m, 1 H), 8.20 (s, 1 H), 7.70- 7.64 (m, 2 H), 7.63 (s, 1 H), 7.55-7.52 (m, 1 H), 7.43- 7.39 (m, 1 H), 7.33-7.31 (m, 1 H), 4.31 (br s, 1 H), 4.08 (br s, 1 H), 2.45-2.43 (m, 2 H), 2.40-2.28 (m, 1 H), 2.27-2.08 (m, 3 H), 1.57- 1.52 (m, 2 H). | m/z 534.1 [M + H]⁺ |
| 189 | 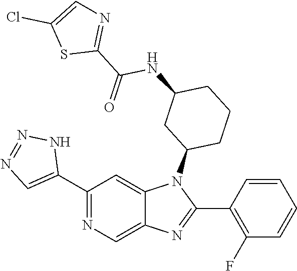 [SMILES: O=C(N[C@H]1CCC[C@@H](n2c(-c3ccccc3F)nc3cnc(-c4cnn[nH]4)cc32)C1)c1ncc(Cl)s1] | (400 MHz, DMSO-d₆) δ 9.12-9.03 (m, 2 H), 8.46- 8.33 (m, 2 H), 8.04 (s, 1 H), 7.77-7.66 (m, 2 H), 7.57- 7.43 (m, 2 H), 4.27-4.16 (m, 1 H), 3.98-3.90 (m, 1 H), 2.65-2.55 (m, 1 H), 2.18- 2.02 (m, 2 H), 1.96-1.78 (m, 3 H), 1.67-1.39 (m, 2 H). | m/z 523.1 [M + H]⁺ |
| 196 | 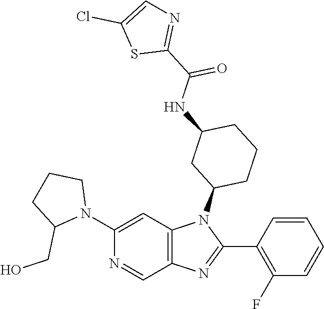 [SMILES: O=C(N[C@H]1CCC[C@@H](n2c(-c3ccccc3F)nc3cnc(N4CCCC4CO)cc32)C1)c1ncc(Cl)s1] | (400 MHz, DMSO-d₆) δ 8.99-8.86 (m, 1 H), 8.54 (s, 1 H), 8.10-8.02 (m, 1 H), 7.71-7.59 (m, 2 H), 7.49- 7.38 (m, 2 H), 6.58-6.46 (m, 1 H), 4.23-4.16 (m, 1 H), 4.07-3.99 (m, 1 H), 3.92- 3.84 (m, 1 H), 3.72-3.65 (m, 1 H), 3.61-3.56 (m, 1 H), 3.54-3.49 (m, 1 H), 3.34- 3.27 (m, 2 H), 2.16-1.74 (m, 10 H), 1.58-1.34 (m, 2 H). | m/z 555.2 [M + H]⁺ |
| 197 | 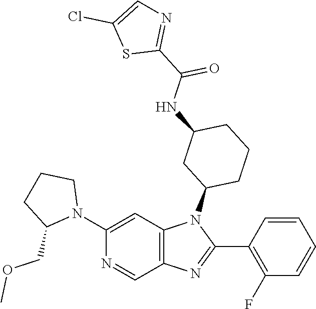 [SMILES: COC[C@@H]1CCCN1c1cc2c(cn1)nc(-c1ccccc1F)n2[C@@H]1CCC[C@H](NC(=O)c2ncc(Cl)s2)C1] | (400 MHz, CD₃OD) δ 8.51 (s, 1 H), 8.17 (s, 1 H), 7.80 (s, 1 H), 7.72-7.60 (m, 2 H), 7.45-7.36 (m, 2 H), 6.76 (s, 1 H), 4.36-4.23 (m, 1 H), 4.21-4.06 (m, 1 H), 3.98- 3.84 (m, 1 H), 3.72-3.59 (m, 2 H), 3.51-3.40 (m, 2 H), 3.40 (s, 3 H), 2.58-2.45 (m, 1 H), 2.29-2.13 (m, 3 H), 2.11-2.04 (m, 3 H), 2.00- 1.91 (m, 3 H), 1.59-1.40 (m, 2 H). | m/z 569.2 [M + H]⁺ |
| 198 | 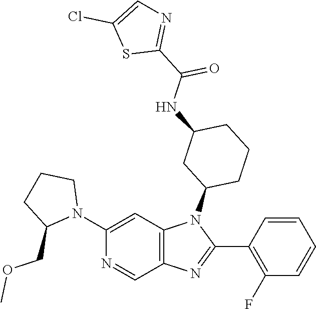 [SMILES: COC[C@H]1CCCN1c1cc2c(cn1)nc(-c1ccccc1F)n2[C@@H]1CCC[C@H](NC(=O)c2ncc(Cl)s2)C1] | (400 MHz, DMSO-d₆) δ 8.90 (d, 1 H), 8.70 (s, 1 H), 8.07 (s, 1 H), 7.79-7.65 (m, 2 H), 7.55-7.44 (m, 2 H), 6.88 (s, 1 H), 4.47-4.41 (m, 1 H), 4.25-4.17 (m, 1 H), 3.96-3.86 (m, 1 H), 3.75- 3.66 (m, 1 H), 3.55-3.40 (m, 3 H), 3.31 (s, 3 H), 2.34-1.99 (m, 7 H), 1.88-1.73 (m, 3 H), 1.55-1.35 (m, 2 H). | m/z 569.2 [M + H]⁺ |
| 199 | 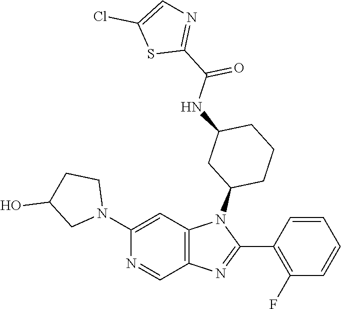 [SMILES: O=C(N[C@H]1CCC[C@@H](n2c(-c3ccccc3F)nc3cnc(N4CCC(O)C4)cc32)C1)c1ncc(Cl)s1] | (400 MHz, DMSO-d₆) δ 9.04-8.96 (m, 1 H), 8.60 (s, 1 H), 8.13 (s, 1 H), 7.79-7.68 (m, 2 H), 7.57-7.44 (m, 2 H), 6.51 (s, 1 H), 5.04 (t, 1 H), 4.51 (s, 1 H), 4.17-4.05 (m, 1 H), 4.01-3.88 (m, 1 H), 3.72-3.55 (m, 3 H), 3.53- 3.45 (m, 1 H), 2.22-2.09 (m, 2 H), 2.06-1.97 (m, 2 H), 1.92-1.81 (m, 3 H), 1.74- 1.52 (m, 2 H), 1.49-1.42 (m, 1 H). | m/z 541.2 [M + H]⁺ |
| 204 | 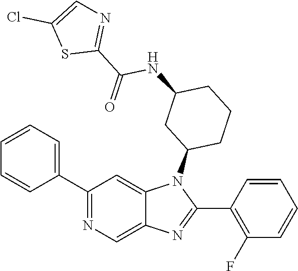 [SMILES: O=C(N[C@H]1CCC[C@@H](n2c(-c3ccccc3F)nc3cnc(-c4ccccc4)cc32)C1)c1ncc(Cl)s1] | (400 MHz, DMSO-d₆) δ 9.10 (s, 1 H), 8.91 (d, 1 H), 8.24-8.16 (m, 3 H), 8.06 (s, 1 H), 7.77-7.69 (m, 2 H), 7.58-7.45 (m, 5 H), 4.29- 4.18 (m, 1 H), 4.01-3.88 (m, 1 H), 2.71-2.56 (m, 1 H), 2.29-2.13 (m, 1 H), 2.08- 1.97 (m, 1 H), 1.94-1.78 (m, 3 H), 1.68-1.55 (m, 1 H), 1.50-1.35 (m, 1 H). | m/z 532.2 [M + H]⁺ |
| 205 | 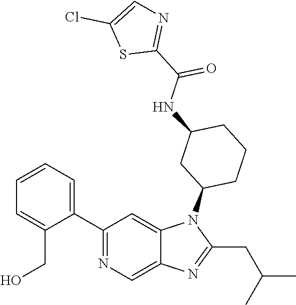 [SMILES: CC(C)Cc1nc2cnc(-c3ccccc3CO)cc2n1[C@@H]1CCC[C@H](NC(=O)c2ncc(Cl)s2)C1] | (400 MHz, CDCl₃) δ 8.94 (s, 1 H), 7.58 (s, 1 H), 7.55 (s, 1 H), 7.52-7.49 (m, 1 H), 7.46-7.33 (m, 3 H), 7.08- 7.06 (m, 1 H), 6.50-6.47 (m, 1 H), 4.40-4.30 (m, 3 H), 4.16-4.11 (m, 1 H), 2.78- 2.76 (m, 2 H), 2.33-2.31 (m, 1 H), 2.30-2.29 (m, 1 H), 2.25-2.11(m, 4 H), 2.07- 2.02 (m, 1 H), 1.98-1.96 (m, 1 H), 1.74-1.51 (m, 1 H), 1.41-1.37 (m, 1 H), 1.03- 1.01 (m, 6 H), 0.98-0.90 (m, 1 H). | m/z 524.2 [M + H]⁺ |
| 206 | 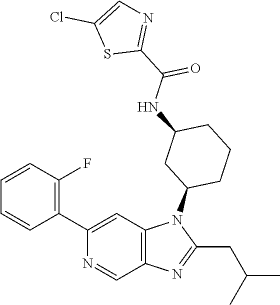 [SMILES: CC(C)Cc1nc2cnc(-c3ccccc3F)cc2n1[C@@H]1CCC[C@H](NC(=O)c2ncc(Cl)s2)C1] | (400 MHz, DMSO-d₆) δ 8.97 (s, 1 H), 8.93 (d, 1 H), 8.04 (s, 1 H), 8.02 (s, 1 H), 7.96-7.91 (m, 1 H), 7.51- 7.44 (m, 1 H), 7.39-7.33 (m, 2 H), 4.70-4.58 (m, 1 H), 4.25-4.11 (m, 1 H), 2.90 (d, 2 H), 2.61-2.53 (m, 1 H), 2.28-2.17 (m, 1 H), 2.15- 2.02 (m, 1 H), 1.99-1.82 (m, 4 H), 1.70-1.52 (m, 2 H), 1.03-0.98 (m, 6 H). | m/z 512.2 [M + H]⁺ |
| 207 | 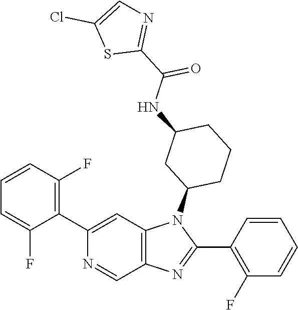 [SMILES: O=C(N[C@H]1CCC[C@@H](n2c(-c3ccccc3F)nc3cnc(-c4c(F)cccc4F)cc32)C1)c1ncc(Cl)s1] | (400 MHz, DMSO-d₆) δ 9.13 (s, 1 H), 8.80 (d, 1 H), 8.04 (s, 1 H), 8.01 (s, 1 H), 7.78-7.70 (m, 2 H), 7.62- 7.45 (m, 3 H), 7.33-7.24 (m, 2 H), 4.28-4.15 (m, 1 H), 3.98-3.86 (m, 1 H), 2.60- 2.52 (m, 1 H), 2.13-1.98 (m, 2 H), 1.92-1.72 (m, 3 H), 1.60-1.31 (m, 2 H). | m/z 568.1 [M + H]⁺ |
| 210 | 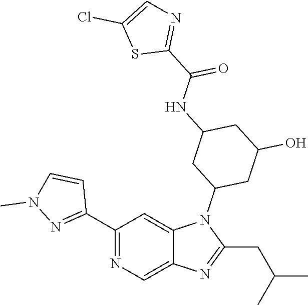 [SMILES: CC(C)Cc1nc2cnc(-c3ccn(C)n3)cc2n1C1CC(O)CC(NC(=O)c2ncc(Cl)s2)C1] | (400 MHz, CD₃OD) δ 8.81 (s, 1 H), 8.27 (s, 1 H), 7.81 (s, 1 H), 7.69-7.68 (m, 1 H), 6.92-6.91 (m, 1 H), 5.08- 5.01 (m, 1 H), 4.63-4.61 (m, 1 H), 4.44 (br s, 1 H), 4.02 (s, 3 H), 3.01-2.89 (m, 2 H), 2.62-2.51 (m, 2 H), 2.31- 2.23 (m, 2 H), 2.18 (br s, 1 H), 2.07-2.01 (m, 1 H), 1.99-1.91 (m, 1 H), 1.09- 1.06 (m, 6 H). | m/z 514.2 [M + H]⁺ |
| | (relative stereochemistry at | | |
| | centers 1 and 3 of cyclohexyl | | |
| | ring is cis with —OH at center 5 | | |
| | trans) | | |
| 211 | 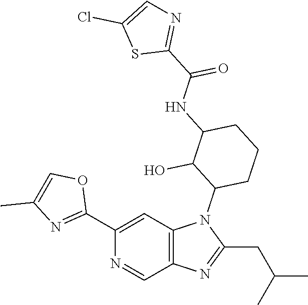 [SMILES: Cc1coc(-c2cc3c(cn2)nc(CC(C)C)n3C2CCCC(NC(=O)c3ncc(Cl)s3)C2O)n1] | (400 MHz, CDCl₃) δ 8.51 (s, 1 H), 7.93-7.88 (m, 2 H), 7.61 (s, 1 H), 7.52 (s, 1 H), 5.58 (s, 1 H), 4.56-4.54 (m, 1 H), 4.35-4.28 (m, 1 H), 4.18-4.15 (m, 1 H), 2.94- 2.91 (dd, 2 H), 2.48 (s, 1 H), 2.40-2.37 (m, 1 H), 2.31 (s, 3 H), 2.06-2.04 (m, 2 H), 1.74-1.71 (m, 3 H), 1.14- 1.10 (m, 6 H). | m/z 515.2 [M + H]⁺ |
| | (relative stereochemistry at | | |
| | centers 1 and 3 of cyclohexyl | | |
| | ring is cis with —OH at center 2 | | |
| | trans) | | |
| 212 | 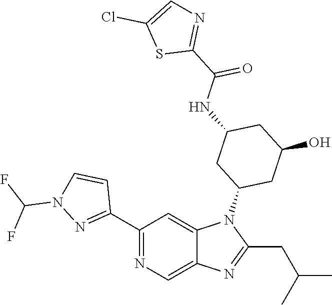 [SMILES: CC(C)Cc1nc2cnc(-c3ccn(C(F)F)n3)cc2n1[C@H]1C[C@H](O)C[C@@H](NC(=O)c2ncc(Cl)s2)C1] | (400 MHz, CD₃OD) δ 8.87 (s, 1 H), 8.37 (s, 1 H), 8.15- 8.14 (m, 1 H), 7.81 (s, 1 H), 7.76-7.45 (m, 1 H), 7.16- 7.15 (m, 1 H), 5.09-4.98 (m, 1 H), 4.67-4.60 (m, 1 H), 4.44 (br s, 1 H), 3.01-2.91 (m, 2 H), 2.61-2.51 (m, 2 H), 2.32-2.23 (m, 2 H), 2.18 (br s, 1 H), 2.05-2.04 (m, 1 H), 1.99-1.91 (m, 1 H), 1.09-1.06 (m, 6 H). | m/z 550.2 [M + H]⁺ (column: DAICEL CHIRALPAK IG (250 mm * 30 mm, 10 um); mobile phase: [0.1% NH₃H₂O MeOH]; B%: 35%-35%, min). Rt = 4.295 min 97.1% ee |
| 213 | 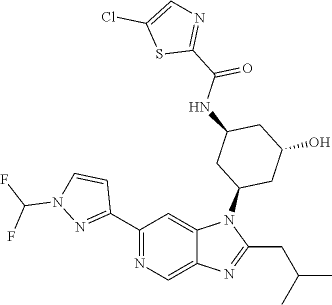 [SMILES: CC(C)Cc1nc2cnc(-c3ccn(C(F)F)n3)cc2n1[C@@H]1C[C@@H](O)C[C@H](NC(=O)c2ncc(Cl)s2)C1] | (400 MHz, CD₃OD) δ 8.87 (s, 1 H), 8.37 (s, 1 H), 8.15- 8.14 (m, 1 H), 7.81 (s, 1 H), 7.75-7.45 (m, 1 H), 7.16- 7.15 (m, 1 H), 5.09-4.98 (m, 1 H), 4.67-4.60 (m, 1 H), 4.44 (br s, 1 H), 3.01-2.91 (m, 2 H), 2.61-2.51 (m, 2 H), 2.32-2.23 (m, 2 H), 2.18 (br s, 1 H), 2.05-2.04 (m, 1 H), 1.99-1.94 (m, 1 H), 1.09-1.06 (m, 6 H). | m/z 550.2 [M + H]⁺ (column: DAICEL CHIRALPAK IG (250 mm * 30 mm, 10 um); mobile phase: [0.1% NH₃H₂O MeOH]; B%: 35%-35%, min). Rt = 3.997 min 100% ee |
| 220 | 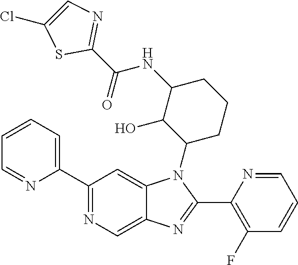 [SMILES: O=C(NC1CCCC(n2c(-c3ncccc3F)nc3cnc(-c4ccccn4)cc32)C1O)c1ncc(Cl)s1] | (400 MHz, DMSO-d₆) δ 9.15 (s, 1 H), 8.98 (d, 1 H), 8.80-8.76 (m, 2 H), 8.68 (d, 1 H), 8.50 (d, 1 H), 8.07-7.95 (m, 3 H), 7.79-7.73 (m, 1 H), 7.48-7.42 (m, 1 H), 5.00 (d, 1 H), 4.56-4.45 (m, 1 H), 4.40-4.32 (m, 1 H), 3.74- 3.64 (m, 1 H), 2.42-2.23 (m, 2 H), 1.95-1.88 (m, 1 H), 1.85-1.75 (m, 2 H), 1.61- 1.41 (m, 1 H). | m/z 550.1 [M + H]⁺ |
| | (relative stereochemistry at | | |
| | centers 1 and 3 of cyclohexyl | | |
| | ring is cis with —OH at center 2 | | |
| | trans) | | |
| 234 | 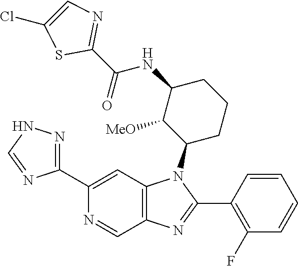 [SMILES: CO[C@@H]1[C@@H](NC(=O)c2ncc(Cl)s2)CCC[C@H]1n1c(-c2ccccc2F)nc2cnc(-c3nc[nH]n3)cc21] | (400 MHz, DMSO-d₆) δ 14.64 (s, 1 H), 9.18-9.16 (m, 2 H), 8.52(s, 1 H), 8.03 (s, 1 H), 7.75-7.67 (m, 2 H), 7.52-7.44 (m, 2 H), 4.25- 4.20 (m, 1 H), 4.08-4.06 (m, 1 H), 3.85-3.32 (m, 1 H), 2.62 (s, 3 H), 2.51-2.50 (m, 2 H), 2.38-2.33 (m, 2 H), 1.83-1.77 (m, 2 H) | m/z 553.2 [M + H]⁺ |
| 235 | 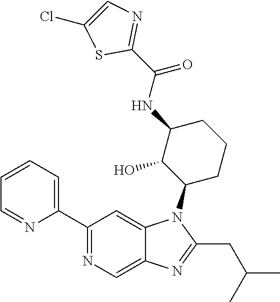 [SMILES: CC(C)Cc1nc2cnc(-c3ccccn3)cc2n1[C@@H]1CCC[C@H](NC(=O)c2ncc(Cl)s2)[C@H]1O] | (400 MHz, CD₃OD) δ 8.96- 8.46 (m, 4 H), 8.02 (br s, 1 H), 7.81 (s, 1 H), 7.63-7.49 (m, 1 H), 4.47 (br s, 1 H), 4.14 (br s, 1 H), 3.03-2.93 (m, 2 H), 2.59-2.56 (m, 1 H), 2.35-2.33 (m, 1 H), 2.14-1.75 (m, 5 H), 1.11- 1.06 (m, 6 H). | m/z 511.2 [M + H]⁺ (column: DIACEL CHIRALPAK AD (250 mm * 30 mm, 10 um); mobile phase: [0.1% NH₃H₂O EtOH]; B%: 40%-40%, min) Rt = 2.342 min 99.5% ee |
| 236 | 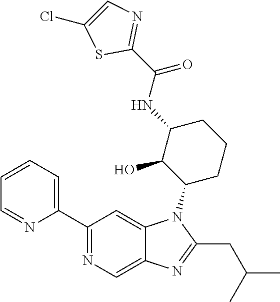 [SMILES: CC(C)Cc1nc2cnc(-c3ccccn3)cc2n1[C@H]1CCC[C@@H](NC(=O)c2ncc(Cl)s2)[C@@H]1O] | (400 MHz, CD₃OD) δ 8.96- 8.49 (m, 4 H), 8.02 (br s, 1 H), 7.81 (s, 1 H), 7.63-7.49 (m, 1 H), 4.47 (br s, 1 H), 4.14 (br s, 1 H), 3.03-2.93 (m, 2 H), 2.59-2.56 (m, 1 H), 2.35 (br s, 1 H), 2.11- 1.75 (m, 5 H), 1.11-1.06 (m, 6 H). | m/z 511.2 [M + H]⁺ (column: DIACEL CHIRALPAK AD (250 mm * 30 mm, 10 um); mobile phase: [0.1% NH₃H₂O EtOH]; B%: 40%-40%, min) Rt = 1.513 min 100% ee |
| 237 | 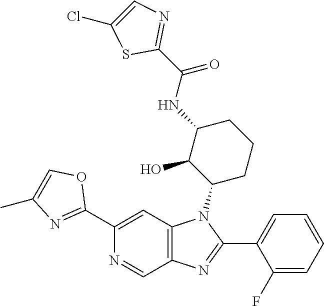 [SMILES: Cc1coc(-c2cc3c(cn2)nc(-c2ccccc2F)n3[C@H]2CCC[C@@H](NC(=O)c3ncc(Cl)s3)[C@@H]2O)n1] | (400 MHz, DMSO-d₆) δ 9.10 (s, 1 H), 8.93 (d, 1 H), 8.41 (s, 1 H), 8.03 (s, 1 H), 8.01 (s, 1 H), 7.75-7.66 (m, 2 H), 7.52-7.42 (m, 2 H), 5.16 (d, 1 H), 4.44-4.35 (m, 1 H), 4.03-3.92 (m, 1 H), 3.77-3.66 (m, 1 H), 2.37- 2.28 (m, 1 H), 2.24 (s, 3 H), 2.14-2.06 (m, 1 H), 1.90- 1.72 (m, 3 H), 1.50-1.35 (m, 1 H). | m/z 553.1 [M + H]⁺ (column: DAICEL CHIRALPAK IG (100 × 4.6 mm, 5 um); mobile phase: [0.05% diethylamine MeOH]; B%: 30%- 30%, min) Rt = 1.595 min 99% ee |
| 238 | 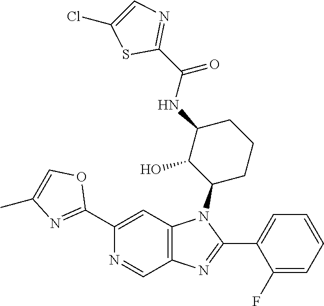 [SMILES: Cc1coc(-c2cc3c(cn2)nc(-c2ccccc2F)n3[C@@H]2CCC[C@H](NC(=O)c3ncc(Cl)s3)[C@H]2O)n1] | (400 MHz, DMSO-d₆) δ 9.10 (s, 1 H), 8.93 (d, 1 H), 8.41 (s, 1 H), 8.07-7.97 (m, 2 H), 7.75-7.67 (m, 2 H), 7.52-7.42 (m, 2 H), 5.16 (d, 1 H), 4.44-4.34 (m, 1 H), 4.05-3.92 (m, 1 H), 3.76- 3.63 (m, 1 H), 2.35-2.27 (m, 1 H), 2.24 (s, 3 H), 2.14-2.07 (m, 1 H), 1.90-1.72 (m, 3 H), 1.48-1.37 (m, 1 H). | m/z 553.1 [M + H]⁺ (column: DAICEL CHIRALPAK IG (100 × 4.6 mm, 5 um); mobile phase: [0.05% diethylamine MeOH]; B%: 40%- 40%, min) Rt = 0.986 min 99% ee |
| 239 | 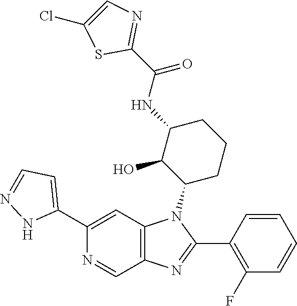 [SMILES: O=C(N[C@@H]1CCC[C@H](n2c(-c3ccccc3F)nc3cnc(-c4ccn[nH]4)cc32)[C@H]1O)c1ncc(Cl)s1] | (400 MHz, DMSO-d₆) δ 9.01 (s, 1 H), 8.87 (s, 1 H), 8.25 (s, 1 H), 8.02 (s, 1 H), 7.76 (s, 1 H), 7.72-7.64 (m, 2 H), 7.51-7.42 (m, 2 H), 6.90 (s, 1 H), 4.38 (t, 1 H), 4.02-3.90 (m, 1 H), 3.75- 3.64 (m, 1 H), 2.45-2.31 (m, 2 H), 2.14-2.04 (m, 1 H), 1.89-1.79 (m, 2 H), 1.49- 1.38 (m, 1 H). | m/z 538.2 [M + H]⁺ (column: DAICEL CHIRALPAK IG (100 × 4.6 mm, 5 um); mobile phase: [0.05% diethylamine IPA]; B%: 40%-40%, min) Rt = 2.741 min 97.9% ee |
| 240 | 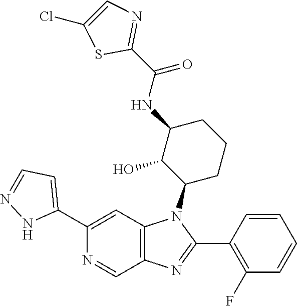 [SMILES: O=C(N[C@H]1CCC[C@@H](n2c(-c3ccccc3F)nc3cnc(-c4ccn[nH]4)cc32)[C@@H]1O)c1ncc(Cl)s1] | (400 MHz, DMSO-d₆) δ 9.01 (s, 1 H), 8.95-8.81 (m, 1 H), 8.25 (s, 1 H), 8.01 (s, 1 H), 7.79-7.63 (m, 3 H), 7.51-7.41 (m, 2 H), 6.89 (s, 1 H), 4.38 (t, 1 H), 4.01-3.89 (m, 1 H), 3.74-3.60 (m, 1 H), 2.42-2.36 (m, 1 H), 2.13- 2.02 (m, 1 H), 1.90-1.77 (m, 3 H), 1.50-1.37 (m, 1 H). | m/z 538.2 [M + H]⁺ (column: DAICEL CHIRALPAK IG (100 × 4.6 mm, 5 um); mobile phase: [0.05% diethylamine IPA]; B%: 40%-40%, min) Rt = 1.464 min 99% ee |
| 241 | 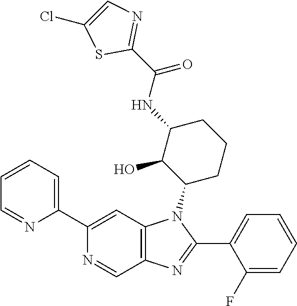 [SMILES: O=C(N[C@@H]1CCC[C@H](n2c(-c3ccccc3F)nc3cnc(-c4ccccn4)cc32)[C@H]1O)c1ncc(Cl)s1] | (400 MHz, DMSO-d₆) δ 9.12 (s, 1 H), 8.97 (d, 1 H), 8.80-8.75 (m, 2 H), 8.50 (d, 1 H), 8.03-7.95 (m, 2 H), 7.75-7.67 (m, 2 H), 7.50- 7.43 (m, 3 H), 5.14 (s, 1 H), 4.39 (t, 1 H), 4.02-3.93 (m, 1 H), 3.76-3.65 (m, 1 H), 2.41-2.33 (m, 1 H), 2.18- 2.07 (m, 1 H), 1.90-1.75 (m, 3 H), 1.51-1.38 (m, 1 H). | m/z 549.1 [M + H]⁺ (column: DAICEL CHIRALPAK IG (100 × 4.6 mm, 5 um); mobile phase: [0.05% diethylamine IPA]; B%: 40%-40%, min) Rt = 2.683 min 96.4% ee |
| 242 | 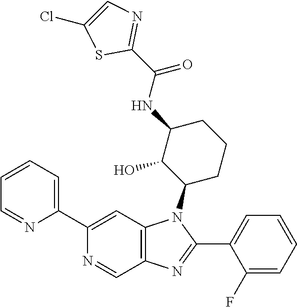 [SMILES: O=C(N[C@H]1CCC[C@@H](n2c(-c3ccccc3F)nc3cnc(-c4ccccn4)cc32)[C@@H]1O)c1ncc(Cl)s1] | (400 MHz, DMSO-d₆) δ 9.11 (s, 1 H), 8.96 (s, 1 H), 8.77 (s, 2 H), 8.51 (s, 1 H), 8.05-7.92 (m, 2 H), 7.76- 7.66 (m, 2 H), 7.52-7.41 (m, 3 H), 5.15 (d, 1 H), 4.48-4.34 (m, 1 H), 4.06-3.92 (m, 1 H), 3.77-3.64 (m, 1 H), 2.39- 2.31 (m, 1 H), 2.16-2.09 (m, 1 H), 1.91-1.76 (m, 3 H), 1.51-1.39 (m, 1 H). | m/z 549.2 [M + H]⁺ (column: DAICEL CHIRALPAK IG (100 × 4.6 mm, 5 um); mobile phase: [0.05% diethylamine IPA]; B%: 40%-40%, min) Rt = 1.230 min 99% ee |
| 245 | 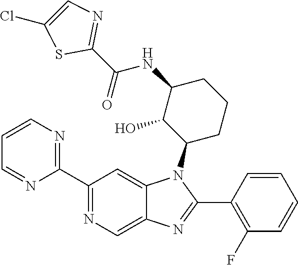 [SMILES: O=C(N[C@H]1CCC[C@@H](n2c(-c3ccccc3F)nc3cnc(-c4ncccn4)cc32)[C@@H]1O)c1ncc(Cl)s1] | (400 MHz, CDCl₃) δ 8.91 (br s, 2 H), 8.72 (br s, 1 H), 7.81 (br s, 1 H), 7.60-7.52 (m, 3 H), 7.38 (br s, 1 H), 7.28-7.27 (m, 1 H), 7.25- 7.24 (m, 1 H), 4.45 (br s, 1 H), 4.16 (br s, 1 H), 3.89 (br s, 1 H), 2.45 (br s, 1 H), 2.31 (br s, 2 H), 2.01 (br s, 1 H), 1.12-0.89 (m, 1 H) | m/z 550.2 [M + H]⁺ |
| 248 | 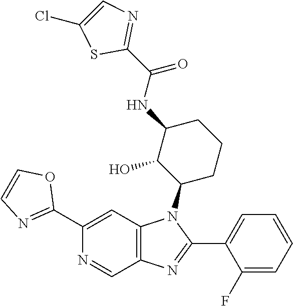 [SMILES: O=C(N[C@H]1CCC[C@@H](n2c(-c3ccccc3F)nc3cnc(-c4ncco4)cc32)[C@@H]1O)c1ncc(Cl)s1] | (400 MHz, DMSO-d₆) δ 9.12 (s, 1 H), 8.90 (d, 1 H), 8.47 (s, 1 H), 8.33 (s, 1 H), 8.03 (s, 1 H), 7.76-7.66 (m, 2 H), 7.51-7.42 (m, 3 H), 5.17 (d, 1 H), 4.45-4.36 (m, 1 H), 4.05-3.93 (m, 1 H), 3.78-3.65 (m, 1 H), 2.31- 2.26 (m, 1 H), 2.15-2.06 (m, 1 H), 1.88-1.74 (m, 3 H), 1.51-1.36 (m, 1 H). | m/z 539.1 [M + H]⁺ (column: DAICEL CHIRALPAK IG (150 × 4.6 mm, 3 um); mobile phase: [0.05% diethylamine EtOH]; B%: 40%-40%, min) Rt = 2.881 min 99.9% ee |
| 249 | 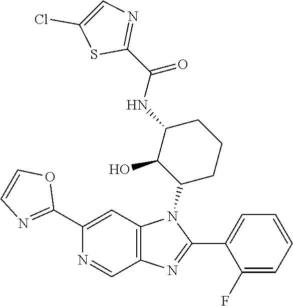 | (400 MHz, DMSO-d₆) δ 9.12 (s, 1 H), 8.90 (d, 1 H), 8.47 (s, 1 H), 8.33 (s, 1 H), 8.03 (s, 1 H), 7.74-7.67 (m, 2 H), 7.52-7.43 (m, 3 H), 5.17 (d, 1 H), 4.45-4.37 (m, 1 H), 4.02-3.94 (m, 1 H), 3.77-3.67 (m, 1 H), 2.30- 2.26 (m, 1 H), 2.13-2.07 (m, 1 H), 1.86-1.76 (m, 3 H), 1.49-1.40 (m, 1 H). | m/z 539.1 [M + H]⁺ (column: DAICEL CHIRALPAK IG (150 × 4.6 mm, 3 um); mobile phase: [0.05% diethylamine EtOH]; B%: 40%-40%, min) Rt = 1.153 min 99% ee |
| 252 | 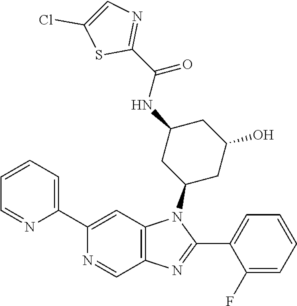 [SMILES: O=C(N[C@H]1C[C@H](O)C[C@@H](n2c(-c3ccccc3F)nc3cnc(-c4ccccn4)cc32)C1)c1ncc(Cl)s1] | (400 MHz, CD₃OD) δ 8.97 (s, 1 H), 8.63 (s, 1 H), 8.57 (br d, 1 H), 8.38-8.37 (m, 1 H), 7.90-7.87 (m, 1 H), 7.67 (s, 1 H), 7.65-7.54 (m, 2 H), 7.40-7.27 (m, 3 H), 4.75- 4.66 (m, 1 H), 4.41-4.22 (m, 2 H), 2.58-2.42 (m, 2 H), 2.22-2.19 (m, 1 H), 2.02- 1.98 (m, 2 H), 1.85-1.78 (m, 1 H). | m/z 549.2 [M + H]⁺ (column: DAICEL CHIRALPAK IG (250 mm * 30 mm, 10 um); mobile phase: [0.1% NH₃H₂O IPA]; B%: 50%-50%, min) Rt = 2.439 min 96.7% ee |
| 253 | 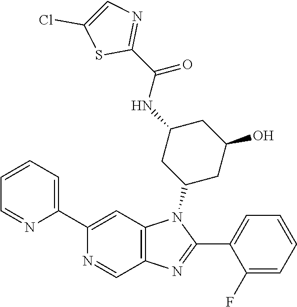 | (400 MHz, CD₃OD) δ 8.97 (s, 1 H), 8.72-8.55 (m, 2 H), 8.38-8.37 (m, 1 H), 7.89- 7.87 (m, 1 H), 7.68 (s, 1 H), 7.66-7.57 (m, 2 H), 7.40- 7.28 (m, 3 H), 4.75-4.65 (m, 1 H), 4.41-4.30 (m, 1 H), 4.27 (br s, 1 H), 2.58-2.42 (m, 2 H), 2.22-2.19 (s, 1 H), 2.202-2.00 (m, 2 H), 1.88- 1.74 (m, 1 H). | m/z 549.2 [M + H]⁺ (column: DAICEL CHIRALPAK IG (250 mm * 30 mm, 10 um); mobile phase: [0.1% NH₃H₂O IPA]; B%: 50%-50%, min) Rt = 1.842 min 100% ee |
| 258 | 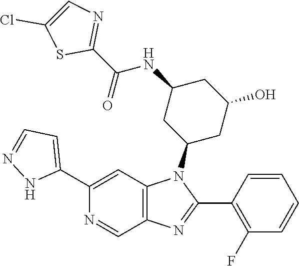 [SMILES: O=C(N[C@H]1C[C@H](O)C[C@@H](n2c(-c3ccccc3F)nc3cnc(-c4ccn[nH]4)cc32)C1)c1ncc(Cl)s1] | (400 MHz, CD₃OD) δ 9.00 (s, 1 H), 8.46 (br s, 1 H), 7.91-7.66 (m, 4 H), 7.58- 7.35 (m, 2 H), 7.05-7.04 (m, 1 H), 4.85-4.74 (m, 1 H), 4.53-4.35 (m, 2 H), 2.67-2.46 (m, 2 H), 2.32- 2.29 (m, 1 H), 2.21-2.05 (m, 2 H), 2.00-1.84 (m, 1 H). | m/z 538.1 [M + H]⁺ (column: (s,s) WHELK-O1 (250 mm * 30 mm, 5 um); mobile phase: [0.1% NH₃H₂O EtOH]; B%: 55%-55%, min) Rt = 6.812 min 100% ee |
| 259 | 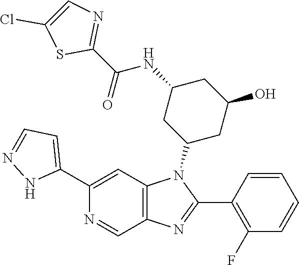 | (400 MHz, CD₃OD) δ 9.00 (br s, 1 H), 8.47 (br s, 1 H), 7.81 (s, 2 H), 7.77-7.64 (m, 2 H), 7.54-7.40 (m, 2 H), 7.04 (br s, 1 H), 4.84-4.71 (m, 1 H), 4.50-4.29 (m, 2 H), 2.69-2.46 (m, 2 H), 2.31 (br s, 1 H), 2.16-2.04 (m, 2 H), 1.96-1.81 (m, 1 H). | m/z 538.1 [M + H]⁺ (column: (s,s) WHELK-O1 (250 mm * 30 mm, 5 um); mobile phase: [0.1% NH₃H₂O EtOH]; B%: 55%-55%, min) Rt = 6.396 min 100% ee |
| 260 | 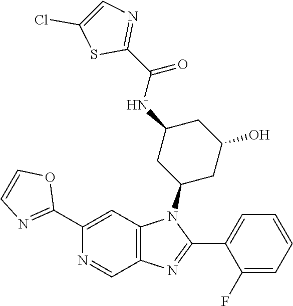 [SMILES: O=C(N[C@H]1C[C@H](O)C[C@@H](n2c(-c3ccccc3F)nc3cnc(-c4ncco4)cc32)C1)c1ncc(Cl)s1] | (400 MHz, CD₃OD) δ 9.08 (s, 1 H), 8.66 (s, 1 H), 8.15 (s, 1 H), 7.81 (s, 1 H), 7.78- 7.70 (m, 2 H), 7.51-7.43 (m, 3 H), 4.78-4.76 (m, 1 H), 4.48-4.42 (m, 1 H), 4.38 (br s, 1 H), 2.59-2.50 (m, 2 H), 2.33-2.30 (m, 1 H), 2.11- 2.08 (m, 2 H), 1.93-1.86 (m, 1 H). | m/z 539.1 [M + H]⁺ (column: DAICEL CHIRALCEL OJ(250 mm * 30 mm, 10 um); mobile phase: [0.1% NH₃H₂O MeOH]; B%: 30%-30%, min) Rt = 4.323 min 100% ee |
| 261 | 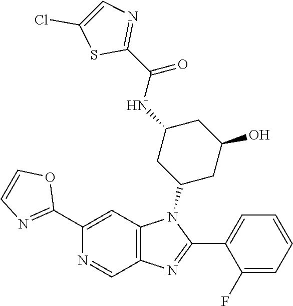 | (400 MHz, CD₃OD) δ 9.08 (s, 1 H), 8.66 (s, 1 H), 8.14 (s, 1 H), 7.80 (s, 1 H), 7.77- 7.69 (m, 2 H), 7.50-7.41 (m, 3 H), 4.83-4.82 (m, 1 H), 4.48-4.42 (m, 1 H), 4.37 (br s, 1 H), 2.58-2.49 (m, 2 H), 2.33-2.30 (m, 1 H), 2.11- 2.08 (m, 2 H), 1.93-1.86 (m, 1 H). | m/z 539.2 [M + H]⁺ (column: DAICEL CHIRALCEL OJ(250 mm * 30 mm, 10 um); mobile phase: [0.1% NH₃H₂O MeOH]; B%: 30%-30%, min) Rt = 3.237 min 100% ee |
| 302 | 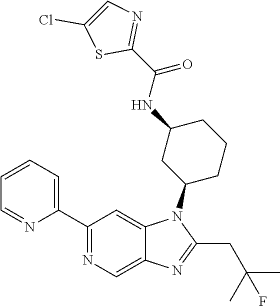 [SMILES: CC(C)(F)Cc1nc2cnc(-c3ccccn3)cc2n1[C@@H]1CCC[C@H](NC(=O)c2ncc(Cl)s2)C1] | (400 MHz, CDCl₃) δ 9.02 (s, 1 H), 8.64-8.63 (m, 1 H), 8.55 (s, 1 H), 8.43-8.41 (m, 1 H), 7.78- 7.76 (m, 1 H), 7.62 (s, 1 H), 7.25-7.22 (m, 1 H), 7.12-7.10 (m, 1 H), 4.55-4.48 (m, 1 H), 4.14-4.12 (m, 1 H), 3.31-3.22 (m, 2 H), 2.33-2.28 (m, 3 H), 2.15-1.97 (m, 3 H), 1.56-1.40 (m, 8 H). | m/z 513.1 [M + H]⁺ |
| 303 | 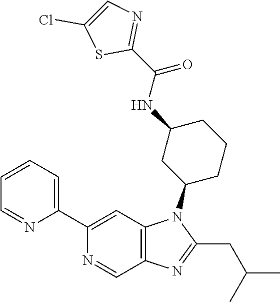 [SMILES: CC(C)Cc1nc2cnc(-c3ccccn3)cc2n1[C@@H]1CCC[C@H](NC(=O)c2ncc(Cl)s2)C1] | (400 MHz, CD₃OD) δ 8.92 (s, 1 H), 8.68 (d, 1 H), 8.65 (s, 1 H), 8.42 (d, 1 H), 8.00-7.94 (m, 1 H), 7.80 (s, 1 H), 7.48-7.43 (m, 1 H), 4.69-4.58 (m, 1 H), 4.25-4.16 (m, 1 H), 3.00-2.90 (m, 2 H), 2.64-2.51 (m, 1 H), 2.48-2.32 (m, 1 H), 2.32-2.16 (m, 2 H), 2.16-2.05 (m, 2 H), 2.05-1.95 (m, 1 H), 1.79-1.68 (m, 2 H), 1.11-1.03 (m, 6 H). | m/z 495.2 [M + H]⁺ |
| 305 | 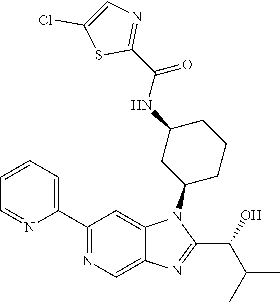 [SMILES: CC(C)[C@@H](O)c1nc2cnc(-c3ccccn3)cc2n1[C@@H]1CCC[C@H](NC(=O)c2ncc(Cl)s2)C1] | (400 MHz, CDCl₃) δ 9.09 (s, 1 H), 8.72-8.71 (m, 1 H), 8.63 (s, 1 H), 8.52-8.50 (m, 1 H), 7.88- 7.84 (m, 1 H), 7.65 (s, 1 H), 7.35-7.28 (m, 2 H), 4.79-4.75 (m, 1 H), 4.65-4.61 (m, 1 H), 3.25-3.23 (m, 1 H), 2.46-2.39 (m, 3 H), 2.25-2.03 (m, 4 H), 1.69-1.63 (m, 4 H), 1.10-1.05 (m, 6 H). | m/z 511.2 [M + H]⁺ |
| 306 | 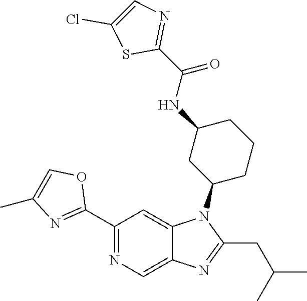 [SMILES: Cc1coc(-c2cc3c(cn2)nc(CC(C)C)n3[C@@H]2CCC[C@H](NC(=O)c3ncc(Cl)s3)C2)n1] | (400 MHz, CD₃OD) δ 8.92 (s, 1 H), 8.54 (s, 1 H), 7.82 (s, 2 H), 4.69-4.65 (m, 1 H), 4.22 (br s, 1 H), 3.00-2.98 (m, 2 H), 2.53-2.50 (m, 1 H), 2.32-2.24 (m, 6 H), 2.13-2.03 (m, 3 H), 1.77-1.72 (m, 2 H), 1.10-1.08 (m, 6 H). | m/z 499.2 [M + H]⁺ |
| 318 | 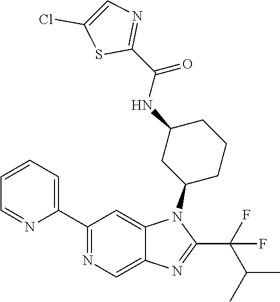 [SMILES: CC(C)C(F)(F)c1nc2cnc(-c3ccccn3)cc2n1[C@@H]1CCC[C@H](NC(=O)c2ncc(Cl)s2)C1] | (400 MHz, CDCl₃) δ 9.18 (s, 1 H), 8.74-8.73 (m, 1 H), 8.67 (s, 1 H), 8.51-8.48 (m, 1 H), 7.86- 7.84 (m, 1 H), 7.63 (s, 1 H), 7.34 (br s, 1 H), 7.18-7.15 (m, 1 H), 4.99-4.93 (m, 1 H), 4.25- 4.20 (m, 1 H), 3.02-2.99 (m, 1 H), 2.47-2.32 (m, 3 H), 2.24- 2.20 (m, 1 H), 2.13-2.09 (m, 2 H), 1.65-1.58 (m, 2 H), 1.30- 1.28 (m, 6 H). | m/z 531.2 [M + H]⁺ |
| 319 | 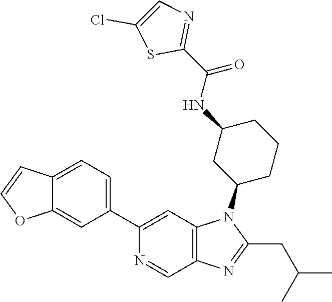 [SMILES: CC(C)Cc1nc2cnc(-c3ccc4ccoc4c3)cc2n1[C@@H]1CCC[C@H](NC(=O)c2ncc(Cl)s2)C1] | (400 MHz, DMSO-d₆) δ 8.95- 8.87 (m, 2 H), 8.36 (s, 1 H), 8.18-8.04 (m, 4 H), 7.77 (d, 1 H), 7.04-6.97 (m, 1 H), 4.72- 4.57 (m, 1 H), 4.29-4.12 (m, 1 H), 2.88 (d, 2 H), 2.73-2.61 (m, 1 H), 2.30-2.13 (m, 2 H), 2.04-1.82 (m, 4 H), 1.67 (s, 2 H), 1.05-0.98 (m, 6 H). | m/z 534.2 [M + H]⁺ |
| 320 | 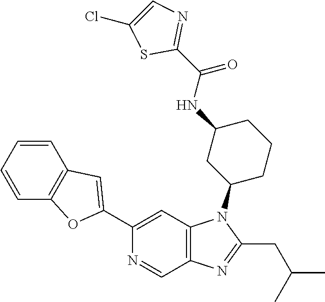 [SMILES: CC(C)Cc1nc2cnc(-c3cc4ccccc4o3)cc2n1[C@@H]1CCC[C@H](NC(=O)c2ncc(Cl)s2)C1] | (400 MHz, DMSO-d₆) δ 9.04 (d, 1 H), 8.95 (s, 1 H), 8.16 (s, 1 H), 8.06 (s, 1 H), 7.74-7.69 (m, 2 H), 7.52 (s, 1 H), 7.40- 7.26 (m, 2 H), 4.76-4.61 (m, 1 H), 4.32-4.14 (m, 1 H), 2.90 (d, 2 H), 2.68-2.56 (m, 1 H), 2.33-2.10 (m, 2 H), 2.02-1.84 (m, 4 H), 1.72-1.61 (m, 2 H), 1.07-1.00 (m, 6 H). | m/z 534.3 [M + H]⁺ |
| 322 | 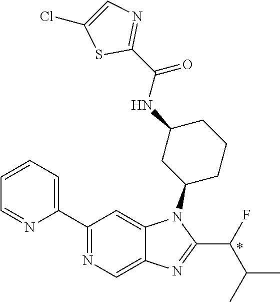 [SMILES: CC(C)C(F)c1nc2cnc(-c3ccccn3)cc2n1[C@@H]1CCC[C@H](NC(=O)c2ncc(Cl)s2)C1] | (400 MHz, CDCl₃) δ 9.16- 9.15 (m, 1 H), 8.72-8.67 (m, 2 H), 8.54-8.52 (m, 1 H), 7.64 (s, 1 H), 7.35-7.33 (m, 2 H), 5.50- 5.36 (m, 1 H), 4.74-4.68 (m, 1 H), 4.26-4.21 (m, 1 H), 2.67- 2.65 (m, 1 H), 2.43-2.04 (m, 8 H), 1.30-1.28 (m, 3 H), 1.05- 0.97 (s, 3 H). | m/z 513.2 [M + H]⁺ |
| 323 | 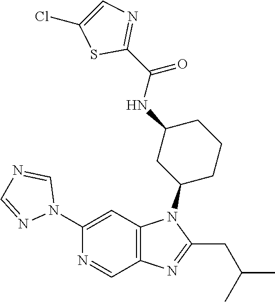 [SMILES: CC(C)Cc1nc2cnc(-n3cncn3)cc2n1[C@@H]1CCC[C@H](NC(=O)c2ncc(Cl)s2)C1] | (400 MHz, CDCl₃) δ 9.22 (s, 1 H), 8.83 (s, 1 H), 8.14 (s, 1 H), 7.98 (s, 1 H), 7.65 (s, 1 H), 7.21-7.19 (m, 1 H), 4.43-4.39 (m, 1 H), 4.23-4.19 (m, 1 H), 2.85-2.84 (m, 2 H), 2.38-2.13 (m, 6 H), 2.02-1.99 (m, 1 H), 1.69-1.62 (m, 2 H), 1.11-1.06 (m, 6 H). | m/z 485.2 [M + H]⁺ |
| 324 | 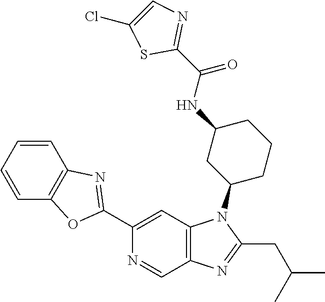 [SMILES: CC(C)Cc1nc2cnc(-c3nc4ccccc4o3)cc2n1[C@@H]1CCC[C@H](NC(=O)c2ncc(Cl)s2)C1] | (400 MHz, DMSO-d₆) δ 9.12 (d, 1 H), 9.04 (s, 1 H), 8.63 (s, 1 H), 8.05 (s, 1 H), 7.89-7.84 (m, 2 H), 7.51-7.42 (m, 2 H), 4.78-4.66 (m, 1 H), 4.29-4.16 (m, 1 H), 2.94 (d, 2 H), 2.72- 2.58 (m, 1 H), 2.32-2.19 (m, 1 H), 2.14-1.88 (m, 5 H), 1.73- 1.61 (m, 2 H), 1.06-0.96 (m, 6 H). | m/z 535.2 [M + H]⁺ |
| 327 | 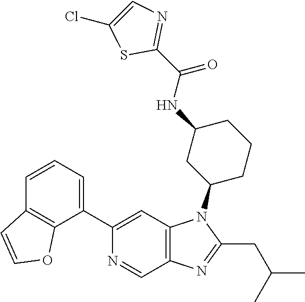 [SMILES: CC(C)Cc1nc2cnc(-c3cccc4ccoc34)cc2n1[C@@H]1CCC[C@H](NC(=O)c2ncc(Cl)s2)C1] | (400 MHz, DMSO-d₆) δ 9.05- 8.97 (m, 2 H), 8.54 (s, 1 H), 8.17 (d, 1 H), 8.11-8.07 (m, 1 H), 8.02 (s, 1 H), 7.73-7.69 (m, 1 H), 7.40 (t, 1 H), 7.10 (d, 1 H), 4.73-4.62 (m, 1 H), 4.24- 4.13 (m, 1 H), 2.90 (d, 2 H), 2.67-2.56 (m, 1 H), 2.28-2.14 (m, 2 H), 2.02-1.88 (m, 4 H), 1.76-1.53 (m, 2 H), 1.06-0.96 (m, 6 H). | m/z 534.3 [M + H]⁺ |
| 329 | 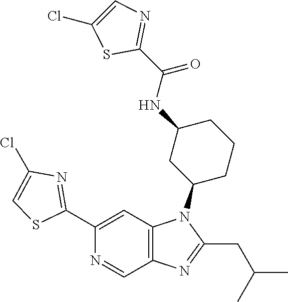 [SMILES: CC(C)Cc1nc2cnc(-c3nc(Cl)cs3)cc2n1[C@@H]1CCC[C@H](NC(=O)c2ncc(Cl)s2)C1] | (400 MHz, CDCl₃) δ 8.98 (s, 1 H), 8.28 (s, 1 H), 7.66 (s, 1 H), 7.28-7.25 (m, 1 H), 7.18 (s, 1 H), 4.43-4.35 (m, 1 H), 4.25- 4.17 (m, 1 H), 2.85-2.83 (m, 2 H), 2.37-2.35 (m, 2 H), 2.32- 2.25 (m, 2 H), 2.23-2.13 (m, 2 H), 2.00-1.98 (m, 1 H), 1.65- 1.60 (m, 2 H), 1.10-1.08 (m, 6 H). | m/z 535.1 [M + H]⁺ |
| 330 | 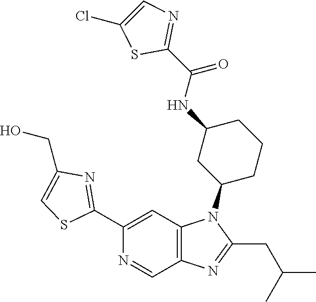 [SMILES: CC(C)Cc1nc2cnc(-c3nc(CO)cs3)cc2n1[C@@H]1CCC[C@H](NC(=O)c2ncc(Cl)s2)C1] | (400 MHz, CDCl₃) δ 8.99 (s, 1 H), 8.36 (s, 1 H), 7.63 (s, 1 H), 7.35-7.33 (m, 1 H), 7.27 (s, 1 H), 4.90 (s, 2 H), 4.42-4.36 (m, 1 H), 4.25-4.18 (m, 1 H), 2.84- 2.82 (m, 2 H), 2.47-2.25 (m, 4 H), 2.23-2.11 (m, 2 H), 1.99- 1.96 (m, 1 H), 1.67-1.60 (m, 2 H), 1.10-1.08 (m, 6 H). | m/z 531.1 [M + H]⁺ |
| 331 | 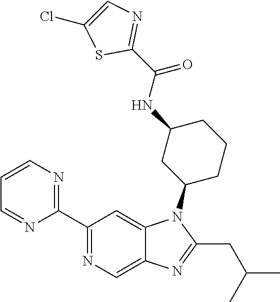 [SMILES: CC(C)Cc1nc2cnc(-c3ncccn3)cc2n1[C@@H]1CCC[C@H](NC(=O)c2ncc(Cl)s2)C1] | (400 MHz, CDCl₃) δ 7.64 (br s, 1 H), 7.37-7.27 (m, 5 H), 4.26-4.18 (m, 2 H), 2.91 (br s, 2 H), 2.28-1.99 (m, 7 H), 1.69- 1.62 (m, 2 H), 1.05 (br s, 6 H). | m/z 496.2 [M + H]⁺ |
| 332 | 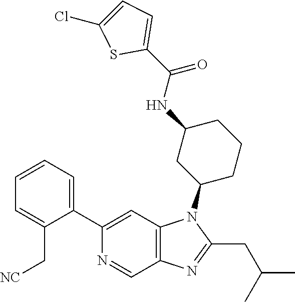 [SMILES: CC(C)Cc1nc2cnc(-c3ccccc3CC#N)cc2n1[C@@H]1CCC[C@H](NC(=O)c2ccc(Cl)s2)C1] | (400 MHz, CD₃OD) δ 8.91 (s, 1 H), 7.84 (s, 1 H), 7.66-7.44 (m, 6 H), 6.98 (d, 1 H), 4.66- 4.56 (m, 1 H), 4.19-4.06 (m, 1 H), 3.96 (s, 2 H), 3.01-2.93 (m, 2 H), 2.48-2.20 (m, 4 H), 2.08-1.93 (m, 3 H), 1.73-1.48 (m, 2 H), 1.08 (d, 6 H). | m/z 532.3 [M + H]⁺ |
| 333 | 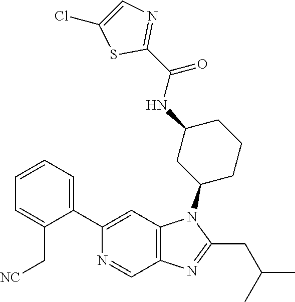 [SMILES: CC(C)Cc1nc2cnc(-c3ccccc3CC#N)cc2n1[C@@H]1CCC[C@H](NC(=O)c2ncc(Cl)s2)C1] | (400 MHz, CD₃OD) δ 8.90 (s, 1 H), 7.89 (s, 1 H), 7.79 (s, 1 H), 7.60-7.54 (m, 2 H), 7.52-7.47 (m, 2 H), 4.66-4.54 (m, 1 H), 4.22-4.11 (m, 1 H), 3.97 (s, 2 H), 3.02-2.94 (m, 2 H), 2.58- 2.48 (m, 1 H), 2.34-2.16 (m, 3 H), 2.09-1.94 (m, 3 H), 1.74- 1.53 (m, 2 H), 1.08 (d, 6 H). | m/z 533.2 [M + H]⁺ |
| 335 | 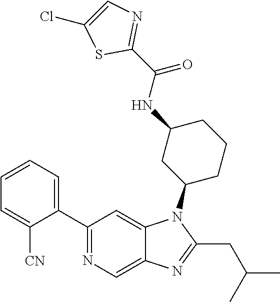 [SMILES: CC(C)Cc1nc2cnc(-c3ccccc3C#N)cc2n1[C@@H]1CCC[C@H](NC(=O)c2ncc(Cl)s2)C1] | (400 MHz, CDCl₃) δ 9.14 (s, 1 H), 8.22-8.17 (m, 2 H), 7.84- 7.82 (m, 1 H), 7.77-7.73 (m, 1 H), 7.65 (s, 1 H), 7.55-7.48 (m, 1 H), 7.33-7.31 (m, 1 H), 4.52- 4.37 (m, 1 H), 4.29-4.16 (m, 1 H), 2.87-2.84 (m, 2 H), 2.39- 2.03 (m, 7 H), 1.65-1.60 (m, 2 H), 1.11-1.08 (m, 6 H). | m/z 519.2 [M + H]⁺ |
| 360 | 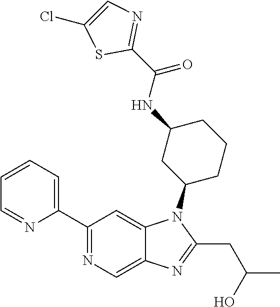 [SMILES: CC(O)Cc1nc2cnc(-c3ccccn3)cc2n1[C@@H]1CCC[C@H](NC(=O)c2ncc(Cl)s2)C1] | (400 MHz, CD₃OD) δ 9.17 (s, 1 H), 8.89 (s, 1 H), 8.85-8.78 (m, 2 H), 8.42-8.38 (m, 1 H), 7.87-7.78 (m, 1 H), 7.76-7.72 (m, 1 H), 4.39-4.26 (m, 1 H), 4.20-4.10 (m, 1 H), 3.42-3.24 (m, 3 H), 2.66-2.50 (m, 1 H), 2.40-2.26 (m, 2 H), 2.15-2.00 (m, 3 H), 1.88-1.64 (m, 2 H), 1.40-1.30 (m, 3 H). | m/z 497.1 [M + H]⁺ |
| 361 | 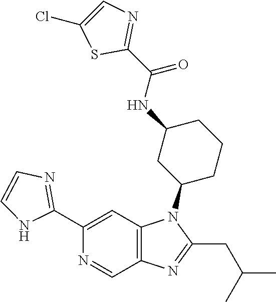 [SMILES: CC(C)Cc1nc2cnc(-c3ncc[nH]3)cc2n1[C@@H]1CCC[C@H](NC(=O)c2ncc(Cl)s2)C1] | (400 MHz, CDCl₃) δ 10.05 (s, 1 H), 8.93 (s, 2 H), 8.34 (s, 1 H), 7.70 (s, 1 H), 7.36-7.30 (m, 1 H), 7.25-7.19 (m, 2 H), 4.42- 4.32 (m, 1 H), 4.25-4.18 (m, 1 H), 2.84-2.72 (m, 2 H), 2.51- 2.49 (m, 1 H), 2.48-2.47 (m, 2 H), 2.25-2.24 (m, 1 H), 2.20- 1.99 (m, 1 H), 1.98-1.97 (m, 2 H), 1.69-1.60 (m, 2 H), 1.10-1.05 (m, 6 H). | m/z 484.1 [M + H]⁺ |
| 362 | 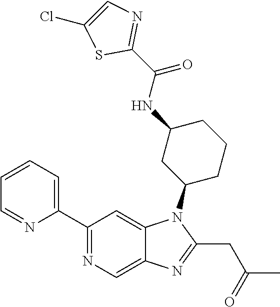 [SMILES: CC(=O)Cc1nc2cnc(-c3ccccn3)cc2n1[C@@H]1CCC[C@H](NC(=O)c2ncc(Cl)s2)C1] | (400 MHz, CDCl₃) δ 9.08-8.91 (m, 1 H), 8.80-8.45 (m, 3 H), 7.86 (s, 1 H), 7.65 (s, 1 H), 7.39-7.31 (m, 1 H), 4.54-3.97 (m, 4 H), 2.41 (s, 5 H), 2.21 (s, 3 H), 2.12 (s, 1 H), 2.04-1.92 (m, 2 H). | m/z 495.1 [M + H]⁺ |
| 374 | 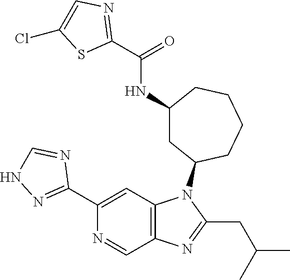 [SMILES: CC(C)Cc1nc2cnc(-c3nc[nH]n3)cc2n1[C@@H]1CCCC[C@H](NC(=O)c2ncc(Cl)s2)C1] | (400 MHz, CDCl₃) δ 9.02 (s, 1 H), 8.27 (s, 1 H), 8.10 (s, 1 H), 7.62 (s, 1 H), 7.17-7.15 (m, 1 H), 4.60-4.54 (m, 1 H), 4.29- 4.21 (m, 1 H), 2.86-2.84 (m, 2 H), 2.73-2.69 (m, 1 H), 2.44 (br s, 1 H), 2.34-2.31 (m, 2 H), 2.25-2.18 (m, 1 H), 2.10-2.05 (m, 1 H), 1.96-1.91 (m, 2 H), 1.86-1.79 (m, 3 H), 1.11-1.08 (m, 6 H). | m/z 499.1 [M + H]⁺; (column: DAICEL CHIRALPAK AD (250 mm * 30 mm, 10 um); mobile phase: [0.1% NH₃H₂O IPA]; B%: 45%-45%, min). Rt = 5.996 min 100% ee |
| 381 | 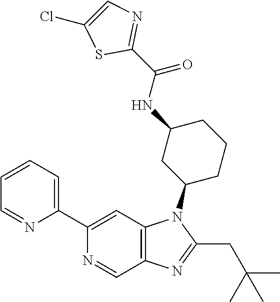 [SMILES: CC(C)(C)Cc1nc2cnc(-c3ccccn3)cc2n1[C@@H]1CCC[C@H](NC(=O)c2ncc(Cl)s2)C1] | (400 MHz, CDCl₃) δ 9.09 (s, 1 H), 8.71-8.69 (m, 1 H), 8.61 (s, 1 H), 8.51-8.49 (m, 1 H), 7.85- 7.83 (m, 1 H), 7.64 (s, 1 H), 7.32-7.22 (m, 2 H), 4.48-4.46 (m, 1 H), 4.22-4.20 (m, 1 H), 2.88 (s, 2 H), 2.42-2.02 (m, 6 H), 1.67-1.62 (m, 1 H), 1.15 (s, 9 H). | m/z 509.2 [M + H]⁺ |
| 383 | 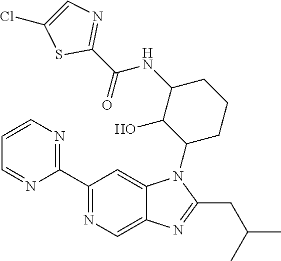 [SMILES: CC(C)Cc1nc2cnc(-c3ncccn3)cc2n1C1CCCC(NC(=O)c2ncc(Cl)s2)C1O] | (400 MHz, CD₃OD) δ 9.00- 8.96 (m, 3 H), 8.89 (br s, 1 H), 7.79 (s, 1 H), 7.51 (br s, 1 H), 4.85-4.84 (m, 1 H), 4.47-4.45 (m, 2 H), 4.15-4.09 (m, 1 H), 3.06-2.89 (m, 3 H), 2.56-2.53 (m, 1 H), 2.38-2.32 (m, 1 H), 2.14-2.01 (m, 3 H), 1.89-1.74 (m, 2 H), 1.10-1.07 (m, 6 H). | m/z 512.2 [M + H]⁺ |
| | (relative stereochemistry at | | |
| | centers 1 and 3 of the cyclohexyl | | |
| | ring is cis with —OH at center 2 | | |
| | trans) | | |
| 391 | 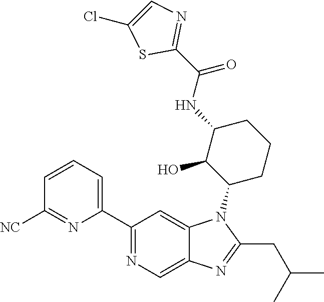 [SMILES: CC(C)Cc1nc2cnc(-c3cccc(C#N)n3)cc2n1[C@H]1CCC[C@@H](NC(=O)c2ncc(Cl)s2)[C@@H]1O] | (400 MHz, CDCl₃) δ 9.00 (br s, 1 H), 8.79 (br s, 1 H), 8.53 (br s, 1 H), 7.98-7.95 (m, 1 H), 7.69-7.67 (m, 1 H), 7.65 (s, 1 H), 7.45-7.42 (m, 1 H), 4.34- 4.32 (m, 2 H), 4.19-4.16 (m, 1 H), 3.76 (br s, 1 H), 2.91-2.89 (m, 2 H), 2.51-2.48 (m, 1 H), 2.36-2.26 (m, 2 H), 2.15-2.04 (m, 2 H), 1.92-1.82 (m, 2 H), 1.11-1.07 (m, 6 H). | m/z 536.2 [M + H]⁺; (column: Phenomenex- Cellulose-2 (250 mm * 30 mm, 5 um); mobile phase: [0.1% NH₃H₂O EtOH]; B%: 40%- 40%, min). Rt = 1.396 min 99.9% ee |
| 392 | 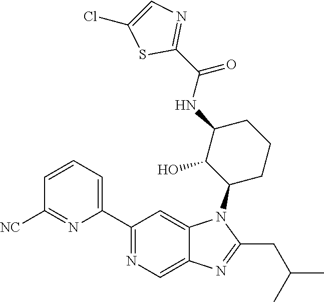 [SMILES: CC(C)Cc1nc2cnc(-c3cccc(C#N)n3)cc2n1[C@@H]1CCC[C@H](NC(=O)c2ncc(Cl)s2)[C@H]1O] | (400 MHz, CDCl₃) δ 8.98 (s, 1 H), 8.78-8.76 (m, 1 H), 8.52 (s, 1 H), 7.97-7.95 (m, 1 H), 7.69- 7.67 (m, 1 H), 7.65 (s, 1 H), 7.46-7.44 (m, 1 H), 4.37-4.31 (m, 2 H), 4.19-4.14 (m, 1 H), 3.76 (br s, 1 H), 2.91-2.89 (m, 2 H), 2.50-2.47 (m, 1 H), 2.36- 2.25 (m, 2 H), 2.13-2.10 (m, 1 H), 2.05 (br s, 1 H), 1.88-1.85 (m, 1 H), 1.63-1.61 (m, 1 H), 1.11-1.06 (m, 6 H). | m/z 536.2 [M + H]⁺; (column: Phenomenex- Cellulose-2 (250 mm * 30 mm, 5 um); mobile phase: [0.1% NH₃H₂O EtOH]; B%: 40%- 40%, min). Rt = 2.050 min 99.7% ee |
| 397 | 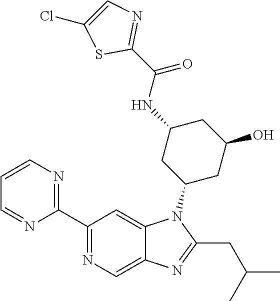 [SMILES: CC(C)Cc1nc2cnc(-c3ncccn3)cc2n1[C@H]1C[C@H](O)C[C@@H](NC(=O)c2ncc(Cl)s2)C1] | (400 MHz, CDCl₃) δ 9.24 (s, 1 H), 8.99-8.93 (m, 2 H), 8.69 (s, 1 H), 7.66 (s, 1 H), 7.35-7.30 (m, 1 H), 7.25-7.18 (m, 1 H), 5.15-5.00 (m, 1 H), 4.80-4.70 (m, 1 H), 4.60 (s, 1 H), 2.94- 2.87 (m, 2 H), 2.62-2.50 (m, 1 H), 2.47-2.29 (m, 4 H), 2.19- 2.13 (m, 1 H), 1.90-1.85 (m, 1 H), 1.13-1.10 (m, 6 H). | m/z 512.2 [M + H]⁺; (column: DAICEL CHIRALCEL OD-H (250 mm * 30 mm, 5 um); mobile phase: [0.1% NH₃H₂O IPA]; B%: 55%-55%, min). Rt = 3.464 min 57.5% ee |
| 398 | 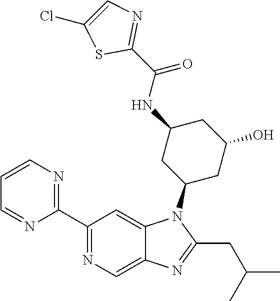 [SMILES: CC(C)Cc1nc2cnc(-c3ncccn3)cc2n1[C@@H]1C[C@@H](O)C[C@H](NC(=O)c2ncc(Cl)s2)C1] | (400 MHz, CDCl₃) δ 9.26 (s, 1 H), 8.99-8.93 (m, 2 H), 8.70 (s, 1 H), 7.66 (s, 1 H), 7.35-7.31 (m, 1 H), 7.24-7.16 (m, 1 H), 5.06 (s, 1 H), 4.74 (s, 1 H), 4.60 (s, 1 H), 2.96-2.87 (m, 2 H), 2.61-2.30 (m, 5 H), 2.22- 2.12 (m, 1 H), 1.90-1.80 (m, 1 H), 1.14-1.09 (m, 6 H). | 512.2 [M + H]⁺; (column: DAICEL CHIRALCEL OD-H (250 mm * 30 mm, 5 um); mobile phase: [0.1% NH₃H₂O IPA]; B%: 55%-55%, min). Rt = 2.323 min 75.2% ee |
| 425 | 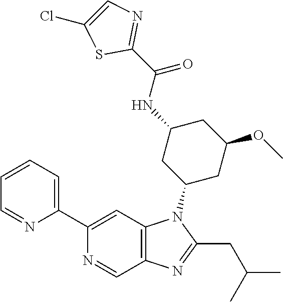 [SMILES: CO[C@@H]1C[C@@H](NC(=O)c2ncc(Cl)s2)C[C@@H](n2c(CC(C)C)nc3cnc(-c4ccccn4)cc32)C1] | (400 MHz, CDCl₃) δ 9.07 (s, 1 H), 8.68-8.66 (m, 1 H), 8.56 (s, 1 H), 8.52-8.50 (m, 1 H), 7.86- 7.82 (m, 1 H), 7.63 (s, 1 H), 7.34-7.28 (m, 2 H), 4.88-4.81 (m, 1 H), 4.67-4.44 (m, 1 H), 3.90 (br s, 1 H), 3.43 (s, 3 H), 2.92-2.80 (m, 2 H), 2.59-2.42 (m, 3 H), 2.38-2.11 (m, 4 H), 1.73-1.64 (m, 1 H), 1.10-1.07 (m, 6 H). | m/z 525.2 [M + H]⁺; (column: REGIS(S,S)WHELK-O1 (250 mm * 25 mm, 10 um); mobile phase: [0.1% NH₃H₂O IPA]; B%: 55%-55%, min). Rt = 3.000 min 100% ee |
| 426 | 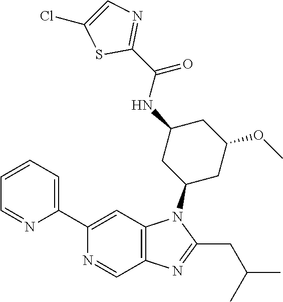 [SMILES: CO[C@H]1C[C@H](NC(=O)c2ncc(Cl)s2)C[C@H](n2c(CC(C)C)nc3cnc(-c4ccccn4)cc32)C1] | (400 MHz, CDCl₃) δ 9.07 (s, 1 H), 8.68-8.66 (m, 1 H), 8.56 (s, 1 H), 8.52-8.50 (m, 1 H), 7.86- 7.82 (m, 1 H), 7.63 (s, 1 H), 7.34-7.28 (m, 2 H), 4.88-4.81 (m, 1 H), 4.67-4.44 (m, 1 H), 3.90 (br s, 1 H), 3.43 (s, 3 H), 2.92-2.80 (m, 2 H), 2.59-2.42 (m, 3 H), 2.38-2.11 (m, 4 H), 1.73-1.64 (m, 1 H), 1.10-1.07 (m, 6 H). | m/z 525.2 [M + H]⁺; (column: REGIS(S,S)WHELK-O1 (250 mm * 25 mm, 10 um); mobile phase: [0.1% NH₃H₂O IPA]; B%: 55%-55%, min). Rt = 4.202 min 94.6% ee |
| 427 | 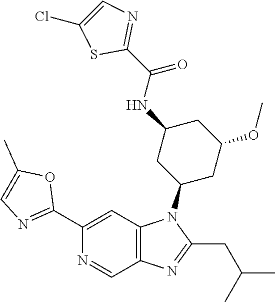 [SMILES: CO[C@H]1C[C@H](NC(=O)c2ncc(Cl)s2)C[C@H](n2c(CC(C)C)nc3cnc(-c4ncc(C)o4)cc32)C1] | (400 MHz, CDCl₃) δ 9.01 (s, 1 H), 8.18 (s, 1 H), 7.57 (s, 1 H), 7.14-7.12 (m, 1 H), 6.83 (s, 1 H), 4.81-4.73 (m, 1 H), 4.48- 4.44 (m, 1 H), 3.81 (br s, 1 H), 3.35 (s, 3 H), 2.80-2.78 (m, 2 H), 2.44-2.40 (m, 4 H), 2.36- 2.26 (m, 3 H), 2.24-2.16 (m, 1 H), 2.11-2.09 (m, 1 H), 1.62- 1.50 (m, 1 H), 1.03-1.00 (m, 6 H). | m/z 529.2 [M + H]⁺; (column: DAICEL CHIRALCEL OD-H (250 mm * 30 mm, 5 um); mobile phase: [0.1% NH₃H₂O MeOH]; B%: 30%-30%, min). Rt = 2.652 min 99.7% ee |
| 428 | 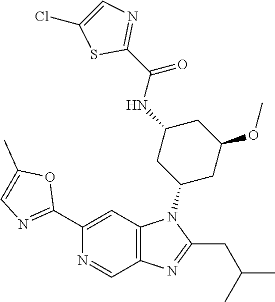 [SMILES: CO[C@@H]1C[C@@H](NC(=O)c2ncc(Cl)s2)C[C@@H](n2c(CC(C)C)nc3cnc(-c4ncc(C)o4)cc32)C1] | (400 MHz, CDCl₃) δ 9.01 (s, 1 H), 8.18 (s, 1 H), 7.57 (s, 1 H), 7.14-7.12 (m, 1 H), 6.83-6.82 (m, 1 H), 4.81-4.73 (m, 1 H), 4.48-4.44 (m, 1 H), 3.81 (br s, 1 H), 3.35 (s, 3 H), 2.80-2.78 (m, 2 H), 2.44-2.40 (m, 4 H), 2.36-2.26 (m, 3 H), 2.24-2.16 (m, 1 H), 2.11-2.09 (m, 1 H), 1.61-1.54 (m, 1 H), 1.03-1.00 (m, 6 H). | m/z 529.2 [M + H]⁺; (column: DAICEL CHIRALCEL OD-H (250 mm * 30 mm, 5 um); mobile phase: [0.1% NH₃H₂O MeOH]; B%: 30%-30%, min). Rt = 3.003 min 92.6% ee |

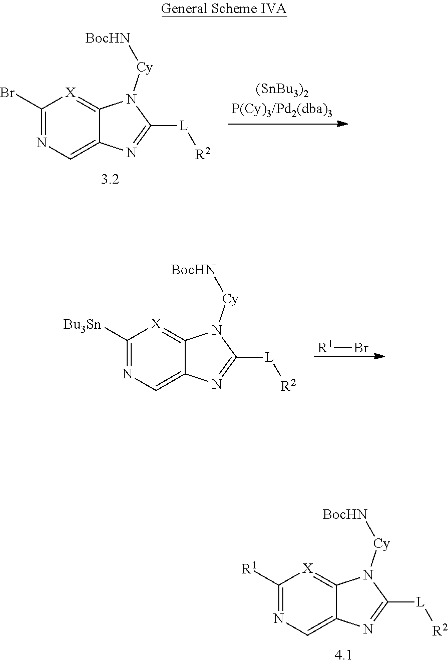

Compounds of formula 4.1 were obtained through the scheme depicted as General Scheme IVA. Similar to Method D in Scheme IV, R¹ groups were installed by converting the bromide in compounds of formula 3.2 with formula 3.2 to a SnBu₃ moiety and then using a Stille coupling with a R¹ heteroaryl or aryl bromide to afford compounds of formula 4.1. Compounds of formula 4.1 were then reacted as set forth in General Scheme IV to form compounds of formulas 4.2 and 4.3.

### Preparation of Example 328 Via General Scheme IVA

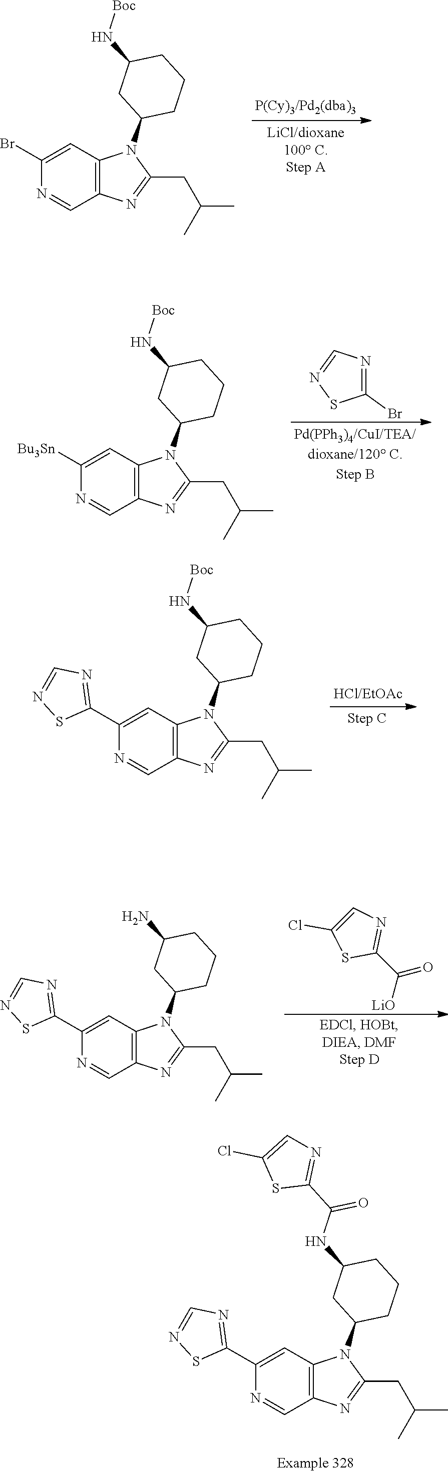

### Step A. Synthesis of tert-butyl ((1S,3R)-3-(2-isobutyl-6-(tributylstannyl)-1H-imidazo[4,5-c]pyridin-1-yl)cyclohexyl)carbamate

A mixture of tert-butyl ((1S,3R)-3-(6-bromo-2-isobutyl-1H-imidazo[4,5-c]pyridin-1-yl)cyclohexyl)carbamate (500 mg, 1.11 mmol), tributyl(tributylstannyl)stannane (3.21 g, 5.54 mmol, 2.77 mL), LiCl (235 mg, 5.54 mmol), tricyclohexylphosphane (62.1 mg, 221 μmol) and Pd₂(dba)₃ (101 mg, 111 μmol) in dioxane (10 mL) was degassed and purged with N₂ for 3 times, and then the reaction mixture was stirred at 100° C. for 2 hrs under N₂ atmosphere. Water (100 mL) was added at 0° C., and then the aqueous portion extracted with EtOAc (100 mL×3). The combined organic layers were dried over Na₂SO₄, filtered, and concentrated to dryness to give tert-butyl ((1S,3R)-3-(2-isobutyl-6-(tributylstannyl)-1H-imidazo[4,5-c]pyridin-1-yl)cyclohexyl)carbamate (600 mg), which was used without further purification. LC-MS: m/z 663.4 [M+H]⁺.

### Step B. Synthesis of tert-butyl ((1S,3R)-3-(2-isobutyl-6-(1,2,4-thiadiazol-5-yl)-1H-imidazo[4,5-c]pyridin-1-yl)cyclohexyl)carbamate

A mixture of tert-butyl ((1S,3R)-3-(2-isobutyl-6-(tributylstannyl)-1H-imidazo[4,5-c]pyridin-1-yl)cyclohexyl)carbamate (300 mg, 453 μmol), 5-bromo-1,2,4-thiadiazole (150 mg, 907 μmol), Pd(PPh₃)₄ (105 mg, 90.7 μmol), CuI (86.4 mg, 453 μmol), TEA (91.8 mg, 907 μmol, 126 μL) in dioxane (7 mL) was degassed and purged with N₂ for 3 times, and then the mixture was stirred at 120° C. for 2 hrs under N₂ atmosphere. The reaction mixture was quenched by addition of sat. aq. NH₄Cl solution (20 mL) at 0° C., and then the aqueous portion extracted with EtOAc (20 mL×3). The combined organic layers were dried over Na₂SO₄, filtered, and concentrated to dryness. The residue was purified by prep-TLC to give tert-butyl ((1S,3R)-3-(2-isobutyl-6-(1,2,4-thiadiazol-5-yl)-1H-imidazo[4,5-c]pyridin-1-yl)cyclohexyl)carbamate (150 mg, 329 μmol, 72.4% yield). LC-MS: 457.4 [M+H]⁺.

### Step C. Synthesis of (1S,3R)-3-(2-isobutyl-6-(1,2,4-thiadiazol-5-yl)-1H-imidazo[4,5-c]pyridin-1-yl)cyclohexan-1-amine

A mixture of tert-butyl ((1S,3R)-3-(2-isobutyl-6-(1,2,4-thiadiazol-5-yl)-1H-imidazo[4,5-c]pyridin-1-yl)cyclohexyl)carbamate (150 mg, 329 μmol), HCl (4 M, 82.1 μL) in EtOAc (2 mL) was degassed and purged with N₂ for 3 times, and then the reaction mixture was stirred at rt for 1 hr under N₂ atmosphere. The reaction mixture was concentrated to dryness to give (1S,3R)-3-(2-isobutyl-6-(1,2,4-thiadiazol-5-yl)-1H-imidazo[4,5-c]pyridin-1-yl)cyclohexan-1-amine (110 mg), which was used without further purification. LC-MS: m/z 357.2 [M+H]⁺

### Step D. Synthesis of 5-chloro-N-((1S,3R)-3-(2-isobutyl-6-(1,2,4-thiadiazol-5-yl)-1H-imidazo[4,5-c]pyridin-1-yl)cyclohexyl)thiazole-2-carboxamide(Example 328)

To a solution of (1S,3R)-3-(2-isobutyl-6-(1,2,4-thiadiazol-5-yl)-1H-imidazo[4,5-c]pyridin-1-yl)cyclohexan-1-amine (60 mg, 168 μmol) in DMF (2 mL) was added EDCI (48.4 mg, 252 μmol), HOBt (34.1 mg, 252 μmol), DIEA (65.3 mg, 505 μmol, 88.0 μL) and (5-chlorothiazole-2-carbonyl)oxylithium (42.8 mg, 252 μmol). The reaction mixture was stirred at rt for 12 hrs. Water (5 mL) was added, followed by an additional 15 mL of water, and the aqueous portion extracted with EtOAc (20 mL×3). The combined organic layers were washed with brine (20 mL×3), dried over Na₂SO₄, filtered, and concentrated to dryness. The residue was purified by column chromatography, and then further purified by reversed-phase HPLC (column: Welch Xtimate C18 150*30 mm*5 um; mobile phase: [water (FA)-ACN]; B %: 48%-78%, 7 min) to give Example 328 (21.4 mg, 24.7% yield). ¹H NMR (400 MHz, CDCl₃) δ 9.03 (s, 1H), 8.75 (s, 1H), 8.33-8.32 (m, 1H), 7.64 (s, 1H), 7.19-7.17 (m, 1H), 4.42-4.38 (m, 1H), 4.23-4.19 (m, 1H), 2.86-2.84 (m, 2H), 2.42-2.39 (m, 1H), 2.33-2.21 (m, 4H), 2.16-2.13 (m, 1H), 2.03-2.01 (m, 1H), 1.69-1.65 (m, 1H), 1.10-1.08 (m, 6H); LC-MS: m/z 502.1 [M+H]⁺.

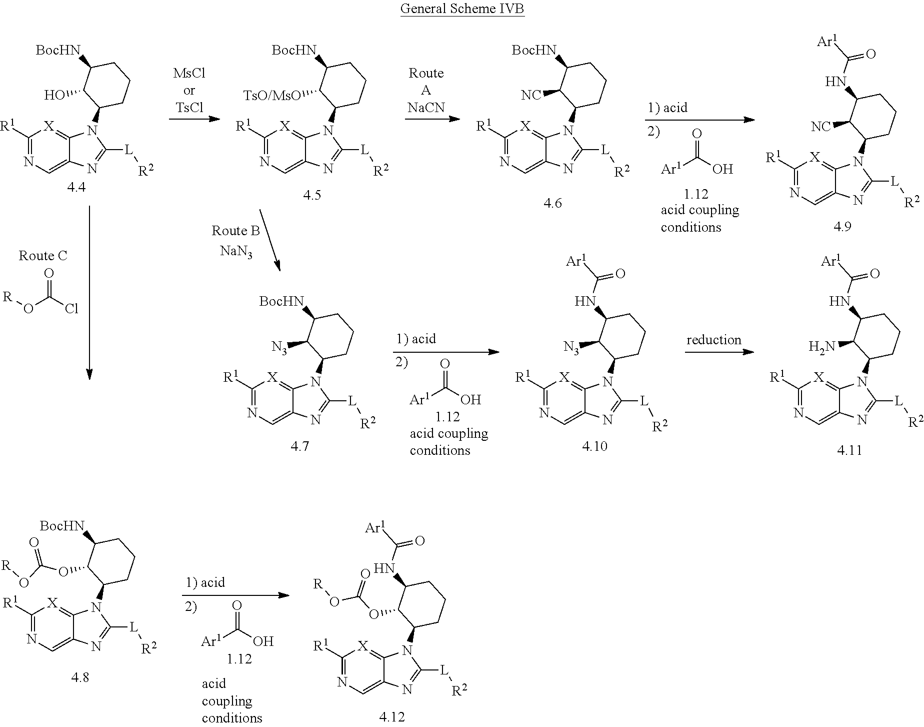

Compounds of formula 4.9, 4.10, 4.11, and 4.12 were obtained through the scheme depicted as General Scheme IVB. Under Routes A and B, the hydroxyl moiety of a compound of formula 4.4 was converted to a tosylate or mesylate moiety by reacting a compound of formula 4.4 with mesyl chloride or tosyl chloride in the presence of base such as TEA or NaH, to afford compounds of formula 4.5. Under Route A, a compound of formula 4.5 is reacted with sodium cyanide in a SN₂ reaction to afford a compound of formula 4.6. Under Route B, a compound of formula 4.5 is reacted with sodium azide in a SN₂ reaction to afford a compound of formula 4.7. Under Route C, a compound of formula 4.5 is reacted with an alkyl carbonochloride to afford a compound of formula 4.8. The Boc protecting groups in compounds of formulas 4.6, 4.7, and 4.8 were removed under acidic conditions, and then Ar¹ groups were then installed by reacting an Ar¹ carboxylic acid of formula 1.12 or a basic salt (i.e., Li, K, or Na) thereof with the unprotected amine in compounds of formulas 4.6, 4.7, and 4.8 using acid coupling conditions known in the art, such as using one of the following reagents—HATU, EDCI, HOBt—along with a base, such as DIPEA (Hunig's base) or TEA to afford compounds of formula 4.9, 4.10, and 4.12. The azido moiety in compounds of formula 4.10 can be reduced to an amino moiety under reduction conditions such as triphenylphosphine in THF/water or Pd/C in the presence of H₂ gas to afford compounds of formula 4.11.

### Preparation of Example 450 Via General Scheme IVB

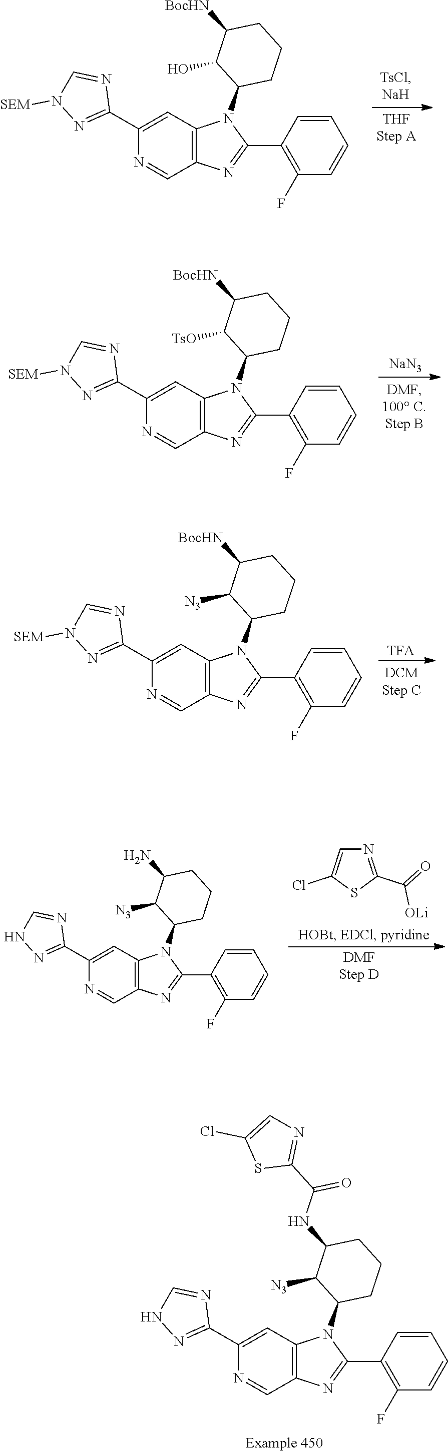

### Step A. Synthesis of (1R,2S,6R)-2-((tert-butoxycarbonyl)amino)-6-(2-(2-fluorophenyl)-6-(1-((2-(trimethylsilyl)ethoxy)methyl)-1H-1,2,4-triazol-3-yl)-1H-imidazo[4,5-c]pyridin-1-yl)cyclohexyl 4-methylbenzenesulfonate

To a solution of tert-butyl ((1S,2R,3R)-3-(2-(2-fluorophenyl)-6-(1-((2-(trimethylsilyl)ethoxy)methyl)-1H-1,2,4-triazol-3-yl)-1H-imidazo[4,5-c]pyridin-1-yl)-2-hydroxycyclohexyl)carbamate (1 g, 2 mmol) in THF (10 mL) was added NaH (80 mg, 2 mmol, 60 wt %) and the reaction mixture was stirred at rt for 1 hr under N₂. Then TsCl (600 mg, 3 mmol) was added and the reaction mixture was stirred at rt for 16 hrs. Sat. aq. NaHCO₃ solution (10 mL) was added, and the aqueous portion extracted with EtOAc (30 mL×3). The combined organic layers were dried over Na₂SO₄, filtered, and concentrated to dryness. The residue was purified by flash column chromatography on silica gel to afford (1R,2S,6R)-2-((tert-butoxycarbonyl)amino)-6-(2-(2-fluorophenyl)-6-(1-((2-(trimethylsilyl)ethoxy)methyl)-1H-1,2,4-triazol-3-yl)-1H-imidazo[4,5-c]pyridin-1-yl)cyclohexyl 4-methylbenzenesulfonate (500 mg, 40% yield). LC-MS: m/z 778 [M+H]⁺.

### Step B. Synthesis of tert-butyl ((1S,2S,3R)-2-azido-3-(2-(2-fluorophenyl)-6-(1-((2-(trimethylsilyl)ethoxy)methyl)-1H-1,2,4-triazol-3-yl)-1H-imidazo[4,5-c]pyridin-1-yl)cyclohexyl)carbamate

To a solution of (1R,2S,6R)-2-((tert-butoxycarbonyl)amino)-6-(2-(2-fluorophenyl)-6-(1-((2-(trimethylsilyl)ethoxy)methyl)-1H-1,2,4-triazol-3-yl)-1H-imidazo[4,5-c]pyridin-1-yl)cyclohexyl 4-methylbenzenesulfonate (100 mg, 129 mol) in DMF (3 mL) was added NaN₃ (16 mg, 257 mol). The reaction mixture was stirred at 100° C. for 16 hrs. Sat. aq. NaHCO₃ solution (20 mL) was added, and the aqueous portion extracted with EtOAc (30 mL×3). The combined organic layers were dried over Na₂SO₄, filtered, and concentrated to dryness. The residue was purified by flash column chromatography on silica gel to afford tert-butyl ((1S,2S,3R)-2-azido-3-(2-(2-fluorophenyl)-6-(1-((2-(trimethylsilyl)ethoxy)methyl)-1H-1,2,4-triazol-3-yl)-1H-imidazo[4,5-c]pyridin-1-yl)cyclohexyl)carbamate (30 mg, 36% yield). LC-MS: m/z 649 [M+H]⁺.

### Step C. Synthesis of (1S,2S,3R)-2-azido-3-(2-(2-fluorophenyl)-6-(1H-1,2,4-triazol-3-yl)-1H-imidazo[4,5-c]pyridin-1-yl)cyclohexan-1-amine

To a solution of tert-butyl ((1S,2S,3R)-2-azido-3-(2-(2-fluorophenyl)-6-(1-((2-(trimethylsilyl)ethoxy)methyl)-1H-1,2,4-triazol-3-yl)-1H-imidazo[4,5-c]pyridin-1-yl)cyclohexyl)carbamate (30 mg, 46 mol) in DCM (1 mL) was added TFA (0.5 mL). The reaction mixture was stirred at rt for 1 hr and then concentrated to dryness to afford (1S,2S,3R)-2-azido-3-(2-(2-fluorophenyl)-6-(1H-1,2,4-triazol-3-yl)-1H-imidazo[4,5-c]pyridin-1-yl)cyclohexan-1-amine (15 mg), which was used without further purification. LC-MS: m/z 419 [M+H]⁺

### Step D. Synthesis of N-((1S,2S,3R)-2-azido-3-(2-(2-fluorophenyl)-6-(1H-1,2,4-triazol-3-yl)-1H-imidazo[4,5-c]pyridin-1-yl)cyclohexyl)-5-chlorothiazole-2-carboxamide (Example 450)

To a solution of (1S,2S,3R)-2-azido-3-(2-(2-fluorophenyl)-6-(1H-1,2,4-triazol-3-yl)-1H-imidazo[4,5-c]pyridin-1-yl)cyclohexan-1-amine (20 mg, 48 mol) in DMF (1 mL) were added Py (11 mg, 0.14 mmol), HOBt (11 mg, 72 mol), EDCI (14 mg, 72 mol) and lithium 5-chlorothiazole-2-carboxylate (10 mg, 57 mol). The reaction mixture was stirred at rt for 16 hrs and then purified by prep-HPLC to afford Example 450 (5 mg, 20% yield). ¹H NMR (400 MHz, DMSO-d₆) δ 14.40 (s, 1H), 9.41 (d, 1H), 8.98 (s, 1H), 8.48 (s, 1H), 7.98 (s, 1H), 7.81-7.69 (m, 2H), 7.63-7.46 (m, 3H), 4.60-4.50 (m, 1H), 4.40-4.36 (m, 1H), 4.04 (s, 1H), 3.17-3.00 (m, 1H), 2.47-2.39 (m, 1H), 2.04-1.98 (m, 1H), 1.87-1.78 (m, 1H), 1.64-1.45 (m, 2H); LC-MS: m/z 564.1 [M+H]⁺.

### Preparation of Example 452 Via General Scheme IVB

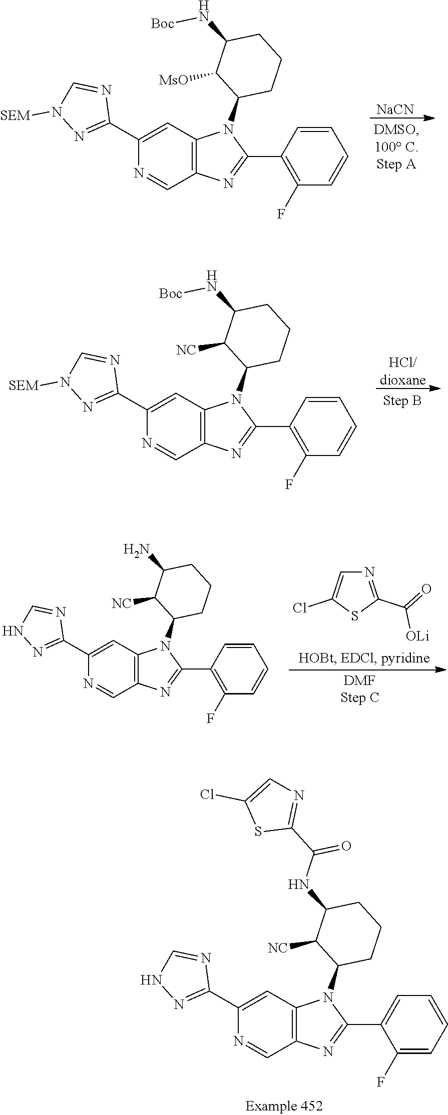

### Step A. Synthesis of tert-butyl ((1S,2S,3R)-2-cyano-3-(2-(2-fluorophenyl)-6-(1-((2-(trimethylsilyl)ethoxy)methyl)-1H-1,2,4-triazol-3-yl)-1H-imidazo[4,5-c]pyridin-1-yl)cyclohexyl)carbamate

To a solution of (1R,2S,6R)-2-((tert-butoxycarbonyl)amino)-6-(2-(2-fluorophenyl)-6-(1-((2-(trimethylsilyl)ethoxy)methyl)-1H-1,2,4-triazol-3-yl)-1H-imidazo[4,5-c]pyridin-1-yl)cyclohexyl methanesulfonate (800 mg, 1.14 mmol) in DMSO (8 mL) were added NaCN (168 mg, 3.42 mmol). The reaction mixture was stirred at 100° C. for 16 hrs under N₂. Water (10 mL) was added, and the aqueous portion extracted with EtOAc (20 mL×3). The combined organic layers were dried over Na₂SO₄, filtered, and concentrated to dryness. The residue was purified by flash column chromatography on silica gel to afford tert-butyl ((1S,2S,3R)-2-cyano-3-(2-(2-fluorophenyl)-6-(1-((2-(trimethylsilyl)ethoxy)methyl)-1H-1,2,4-triazol-3-yl)-1H-imidazo[4,5-c]pyridin-1-yl)cyclohexyl)carbamate (350 mg, 49% yield). LC-MS: m/z 633 [M+H]⁺.

### Step B. Synthesis of (1S,2S,6R)-2-amino-6-(2-(2-fluorophenyl)-6-(1H-1,2,4-triazol-3-yl)-1H-imidazo[4,5-c]pyridin-1-yl)cyclohexane-1-carbonitrile

A mixture of tert-butyl ((1S,2S,3R)-2-cyano-3-(2-(2-fluorophenyl)-6-(1-((2-(trimethylsilyl)ethoxy)methyl)-1H-1,2,4-triazol-3-yl)-1H-imidazo[4,5-c]pyridin-1-yl)cyclohexyl)carbamate (300 mg, 474 mol) in HCl/dioxane (2 mL, 4 M) was stirred at rt for 1 hr under N₂. The reaction mixture was then concentrated to dryness to afford (1S,2S,6R)-2-amino-6-(2-(2-fluorophenyl)-6-(1H-1,2,4-triazol-3-yl)-1H-imidazo[4,5-c]pyridin-1-yl)cyclohexane-1-carbonitrile (190 mg), which was used without further purification. LC-MS: m/z 403 [M+H]⁺.

### Step C. Synthesis of 5-chloro-N-((1S,2S,3R)-2-cyano-3-(2-(2-fluorophenyl)-6-(1H-1,2,4-triazol-3-yl)-1H-imidazo[4,5-c]pyridin-1-yl)cyclohexyl)thiazole-2-carboxamide (Example 452)

To a solution of (1S,2S,6R)-2-amino-6-(2-(2-fluorophenyl)-6-(1H-1,2,4-triazol-3-yl)-1H-imidazo[4,5-c]pyridin-1-yl)cyclohexane-1-carbonitrile (190 mg, 472 mol) and lithium 5-chlorothiazole-2-carboxylate (120 mg, 708 mol) in DMF (3 mL) were added HOBt (108 mg, 708 mol), EDCI (136 mg, 708 mol) and Py (112 mg, 1.42 mmol). The reaction mixture was stirred at rt for 16 hrs and was then purified by prep-HPLC to afford Example 452 (30 mg, 12% yield). ¹H NMR (400 MHz, DMSO-d₆) δ 14.42 (s, 1H), 9.59 (d, 1H), 8.98 (s, 1H), 8.49 (s, 1H), 8.02 (s, 1H), 7.80-7.70 (m, 2H), 7.61 (s, 1H), 7.57-7.47 (m, 2H), 4.67-4.53 (m, 2H), 3.33-3.28 (m, 1H), 3.21-3.08 (m, 1H), 2.59-2.53 (m, 1H), 2.14-1.89 (m, 2H), 1.79-1.49 (m, 2H); LC-MS: m/z 548.1 [M+H]⁺.

General Scheme IVB set forth above was used to synthesize the following compounds by using appropriate starting materials:

| Ex. | | | |
|---|---|---|---|
| No. | Structure | ¹H NMR | LC-MS: m/z and % ee |
| 448 | 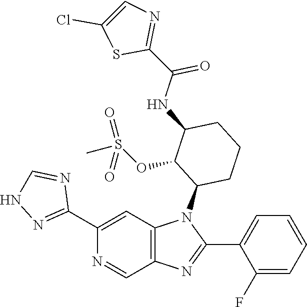 [SMILES: CS(=O)(=O)O[C@@H]1[C@@H](NC(=O)c2ncc(Cl)s2)CCC[C@H]1n1c(-c2ccccc2F)nc2cnc(-c3nc[nH]n3)cc21] | (400 MHz, DMSO) δ 14.71 (s, 1H), 9.23 (d, 1H), 9.13 (s, 1H), 8.46 (s, 1H), 8.16-8.01 (m, 2H), 7.78-7.67 (m, 2H), 7.54-7.46 (m, 2H), 5.48 (t, 1H), 4.45 (t, 1H), 4.38-4.21 (m, 1H), 2.58 (s, 3H), 2.48-2.38 (m, 1H), 2.29-2.15 (m, 1H), 1.97-1.80 (m, 3H), 1.75-1.58 (m, 1H). | 617.0 [M + H]⁺ (column: ChiralPak IG (250 mm*4.6 mm, 5 um); mobile phase: [0.05% DEA MeOH]; B %: 8 min @ 30%); Rt = 2.235 min >99% ee |

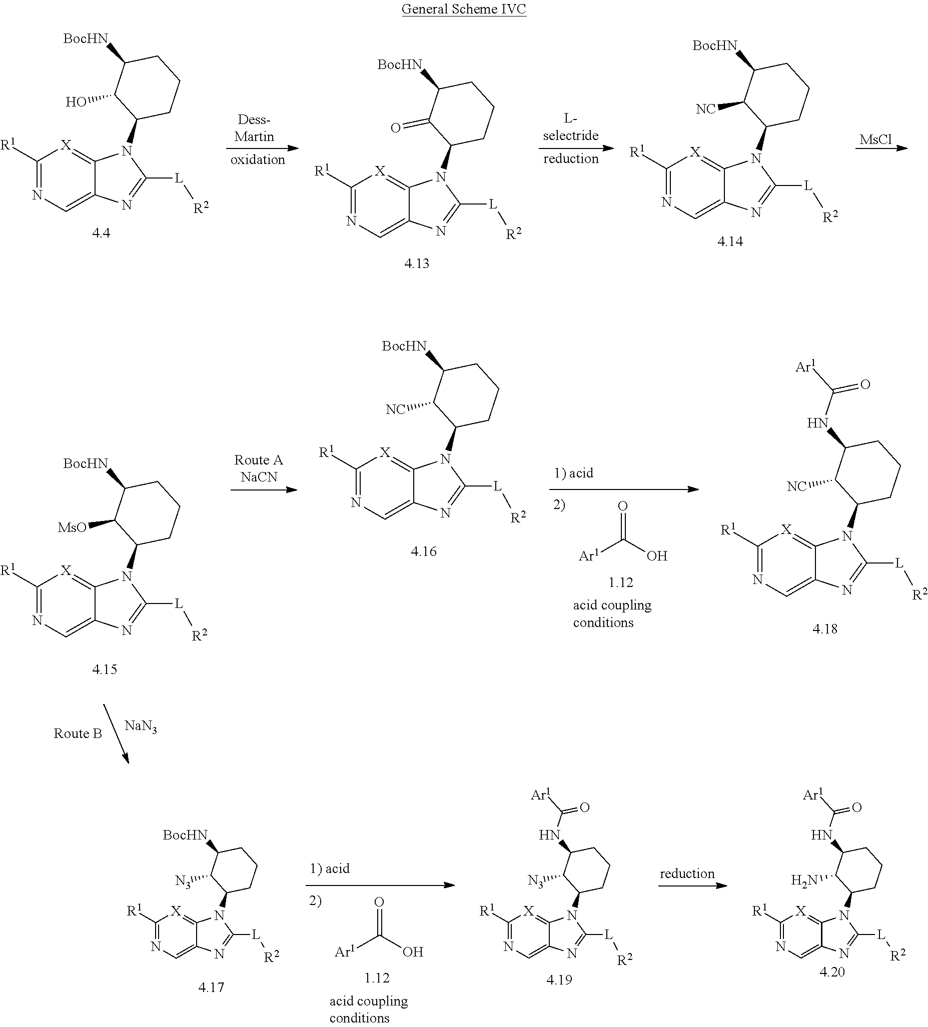

Compounds of formula 4.18, 4.19, and 4.20 were obtained through the scheme depicted as General Scheme IVC. The stereochemistry of the hydroxyl moiety of a compound of formula 4.4 was inversed by first using a Dess-Martin oxidization reaction to afford a compound of formula 4.13, followed by a reduction reaction using L-selectride to afford compounds of formula 4.14. The hydroxyl moiety of compounds of formula 4.14 was converted to a mesylate moiety by reacting a compound of formula 4.14 with mesyl chloride in the presence of base to afford compounds of formula 4.15. Under Route A, a compound of formula 4.15 is reacted with sodium cyanide in a SN₂ reaction to afford a compound of formula 4.16. Under Route B, a compound of formula 4.15 is reacted with sodium azide in a SN₂ reaction to afford a compound of formula 4.17. The Boc protecting groups in compounds of formulas 4.16 and 4.17 were removed under acidic conditions, and then Ar¹ groups were then installed by reacting an Ar¹ carboxylic acid of formula 1.12 or a basic salt (i.e., Li, K, or Na) thereof with the unprotected amine in compounds of formulas 4.16 and 4.17 using acid coupling conditions known in the art, such as using one of the following reagents—HATU, EDCI, HOBt—along with a base, such as DIPEA (Hunig's base) or TEA to afford compounds of formula 4.18 and 4.19. The azido moiety in compounds of formula 4.19 can be reduced to an amino moiety under reduction conditions such as triphenylphosphine in THF/water or Pd/C in the presence of H₂ gas to afford compounds of formula 4.20.

### Preparation of Example 449 Via General Scheme IVC

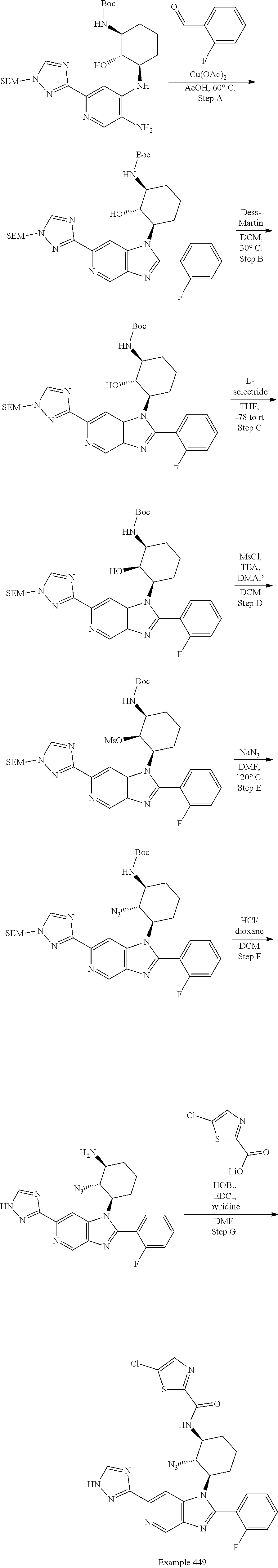

### Step A. Synthesis of tert-butyl ((1S,3R)-3-(2-(2-fluorophenyl)-6-(1-((2-(trimethylsilyl)ethoxy)methyl)-1H-1,2,4-triazol-3-yl)-1H-imidazo[4,5-c]pyridin-1-yl)-2-oxocyclohexyl)carbamate

To a mixture of tert-butyl ((1R,2R,3S)-3-((5-amino-2-(1-((2-(trimethylsilyl)ethoxy)methyl)-1H-1,2,4-triazol-3-yl)pyridin-4-yl)amino)-2-hydroxycyclohexyl)carbamate (8 g, 15.4 mmol) in AcOH (60 mL) was added 2-fluorobenzaldehyde (2.87 g, 23.1 mmol) and then the reaction mixture was stirred at 60° C. for 3 hrs. Then Cu(OAc)₂ (2.8 g, 15.4 mmol) was added, and the reaction mixture was stirred at 60° C. for 24 hrs. The reaction mixture was allowed to cool and was then filtered. Water (40 mL) was added to the filtrate, and the aqueous portion extracted with EtOAc (60 mL×3). The combined organic layers were washed with brine (15 mL), dried over Na₂SO₄, and concentrated to dryness. The residue was purified by flash column chromatography on silica gel to afford tert-butyl ((1S,3R)-3-(2-(2-fluorophenyl)-6-(1-((2-(trimethylsilyl)ethoxy)methyl)-1H-1,2,4-triazol-3-yl)-1H-imidazo[4,5-c]pyridin-1-yl)-2-oxocyclohexyl)carbamate (6.5 g, 58% yield). LC-MS: m/z 624 [M+H]⁺.

### Step B. Synthesis of tert-butyl ((1S,3R)-3-(2-(2-fluorophenyl)-6-(1-((2-(trimethylsilyl)ethoxy)methyl)-1H-1,2,4-triazol-3-yl)-1H-imidazo[4,5-c]pyridin-1-yl)-2-oxocyclohexyl)carbamate

To a solution of tert-butyl ((1S,2R,3R)-3-(2-(2-fluorophenyl)-6-(1-((2-(trimethylsilyl)ethoxy)methyl)-1H-1,2,4-triazol-3-yl)-1H-imidazo[4,5-c]pyridin-1-yl)-2-hydroxycyclohexyl)carbamate (3 g, 4.81 mmol) in DCM (30 mL) was added Dess-Martin periodinane (4.08 g, 9.62 mmol, 2.0 eq.) at 0° C. The reaction mixture was stirred at 30° C. for 16 hrs and then concentrated to dryness. The residue was purified by flash column chromatography on silica gel to afford tert-butyl ((1S,3R)-3-(2-(2-fluorophenyl)-6-(1-((2-(trimethylsilyl)ethoxy)methyl)-1H-1,2,4-triazol-3-yl)-1H-imidazo[4,5-c]pyridin-1-yl)-2-oxocyclohexyl)carbamate (2.5 g, 71% yield). LC-MS: m/z 622 [M+H]⁺.

### Step C. Synthesis of tert-butyl ((1S,3R)-3-(2-(2-fluorophenyl)-6-(1-((2-(trimethylsilyl)ethoxy)methyl)-1H-1,2,4-triazol-3-yl)-1H-imidazo[4,5-c]pyridin-1-yl)-2-oxocyclohexyl)carbamate

To a solution of tert-butyl ((1S,3R)-3-(2-(2-fluorophenyl)-6-(1-((2-(trimethylsilyl)ethoxy)methyl)-1H-1,2,4-triazol-3-yl)-1H-imidazo[4,5-c]pyridin-1-yl)-2-oxocyclohexyl)carbamate (2 g, 3.22 mmol) in THF (20 mL) was added L-selectride (9.7 mL, 9.65 mmol, 1M in THF) at −78° C. under N₂ atmosphere. The mixture was stirred at rt for 16 hrs. Sat. aq. NH₄Cl solution (10 mL) was added, and the aqueous portion extracted with EtOAc (40 mL×3). The combined organic layers were dried over Na₂SO₄, filtered, and concentrated to dryness. The residue was purified by flash column chromatography on silica gel to afford tert-butyl ((1S,3R)-3-(2-(2-fluorophenyl)-6-(1-((2-(trimethylsilyl)ethoxy)methyl)-1H-1,2,4-triazol-3-yl)-1H-imidazo[4,5-c]pyridin-1-yl)-2-oxocyclohexyl)carbamate (1.1 g, 49% yield). LC-MS: m/z 624 [M+H]⁺.

### Step D. Synthesis of (1S,2S,6R)-2-((tert-butoxycarbonyl)amino)-6-(2-(2-fluorophenyl)-6-(1-((2-(trimethylsilyl)ethoxy)methyl)-1H-1,2,4-triazol-3-yl)-1H-imidazo[4,5-c]pyridin-1-yl)cyclohexyl methanesulfonate

To a solution of tert-butyl ((1S,2S,3R)-3-(2-(2-fluorophenyl)-6-(1-((2-(trimethylsilyl)ethoxy)methyl)-1H-1,2,4-triazol-3-yl)-1H-imidazo[4,5-c]pyridin-1-yl)-2-hydroxycyclohexyl)carbamate (1 g, 1.60 mmol) in THF (15 mL) were added TEA (486 mg, 4.81 mmol), MsCl (551 mg, 4.81 mmol) and DMAP (294 mg, 2.41 mmol) at 30° C. Then the mixture was stirred at 30° C. for 3 hrs. Sat. aq. NH₄Cl solution (30 mL) was added, and the aqueous portion extracted with EtOAc (40 mL×3). The combined organic layers were dried over Na₂SO₄, filtered, and concentrated to dryness. The residue was purified by flash column chromatography on silica gel to afford (1S,2S,6R)-2-((tert-butoxycarbonyl)amino)-6-(2-(2-fluorophenyl)-6-(1-((2-(trimethylsilyl)ethoxy)methyl)-1H-1,2,4-triazol-3-yl)-1H-imidazo[4,5-c]pyridin-1-yl)cyclohexyl methanesulfonate (1 g, 76% yield). LC-MS: m/z 702 [M+H]⁺.

### Step E. Synthesis of tert-butyl ((1S,2R,3R)-2-azido-3-(2-(2-fluorophenyl)-6-(1-((2-(trimethylsilyl)ethoxy)methyl)-1H-1,2,4-triazol-3-yl)-1H-imidazo[4,5-c]pyridin-1-yl)cyclohexyl)carbamate

To a solution of (1S,2S,6R)-2-((tert-butoxycarbonyl)amino)-6-(2-(2-fluorophenyl)-6-(1-((2-(trimethylsilyl)ethoxy)methyl)-1H-1,2,4-triazol-3-yl)-1H-imidazo[4,5-c]pyridin-1-yl)cyclohexyl methanesulfonate (600 mg, 855 mol) in DMF (10 mL) was added sodium azide (167 mg, 2.56 mmol). The reaction mixture was stirred at 120° C. for 72 hrs. The reaction mixture was allowed to cool, and sat. aq. NH₄Cl solution (20 mL) was added. The aqueous portion was extracted with EtOAc (20 mL×3). The combined organic layers were dried over Na₂SO₄, filtered, and concentrated to dryness. The residue was purified by prep-HPLC to afford tert-butyl ((1S,2R,3R)-2-azido-3-(2-(2-fluorophenyl)-6-(1-((2-(trimethylsilyl)ethoxy)methyl)-1H-1,2,4-triazol-3-yl)-1H-imidazo[4,5-c]pyridin-1-yl)cyclohexyl)carbamate (130 mg, 20% yield). LC-MS: m/z 649 [M+H]⁺.

### Step F. Synthesis of (1S,2S,6R)-2-amino-6-(2-isobutyl-6-(1H-1,2,4-triazol-3-yl)-1H-imidazo[4,5-c]pyridin-1-yl)cyclohexan-1-ol

A solution of tert-butyl ((1S,2R,3R)-2-azido-3-(2-(2-fluorophenyl)-6-(1-((2-(trimethylsilyl)ethoxy)methyl)-1H-1,2,4-triazol-3-yl)-1H-imidazo[4,5-c]pyridin-1-yl)cyclohexyl)carbamate (130 mg, 200 mol) in HCl/dioxane (3 mL, 4M) was stirred at rt for 2 hrs and then concentrated to dryness to afford (1S,2S,6R)-2-amino-6-(2-isobutyl-6-(1H-1,2,4-triazol-3-yl)-1H-imidazo[4,5-c]pyridin-1-yl)cyclohexan-1-ol (80 mg), which was used without further purification. LC-MS: m/z 419 [M+H]⁺.

### Step G. Synthesis of N-((1S,2R,3R)-2-azido-3-(2-(2-fluorophenyl)-6-(1H-1,2,4-triazol-3-yl)-1H-imidazo[4,5-c]pyridin-1-yl)cyclohexyl)-5-chlorothiazole-2-carboxamide (Example 449)

To a mixture of (1S,2R,3R)-2-azido-3-(2-(2-fluorophenyl)-6-(1H-1,2,4-triazol-3-yl)-1H-imidazo[4,5-c]pyridin-1-yl)cyclohexan-1-amine (100 mg, 239 mol) and lithium 5-chlorothiazole-2-carboxylate (61 mg, 358 mol) in DMF (3 mL) were added pyridine (1 mL), HOBt (55 mg, 358 mol) and EDCI (69 mg, 358 mol). The reaction mixture was stirred at rt for 16 hrs and then purified by prep-HPLC to afford Example 449 (30 mg, 22% yield). ¹H NMR (400 MHz, CD₃OD) δ 9.34 (s, 1H), 8.95 (s, 1H), 8.83 (s, 1H), 7.85-7.74 (m, 3H), 7.56-7.42 (m, 2H), 4.53 (t, 1H), 4.31-4.17 (m, 1H), 4.11-3.99 (m, 1H), 2.71-2.54 (m, 1H), 2.48-2.35 (m, 1H), 2.13-2.01 (m, 2H), 1.98-1.84 (m, 1H), 1.71-1.52 (m, 1H); LC-MS: m/z 564.1 [M+H]⁺.

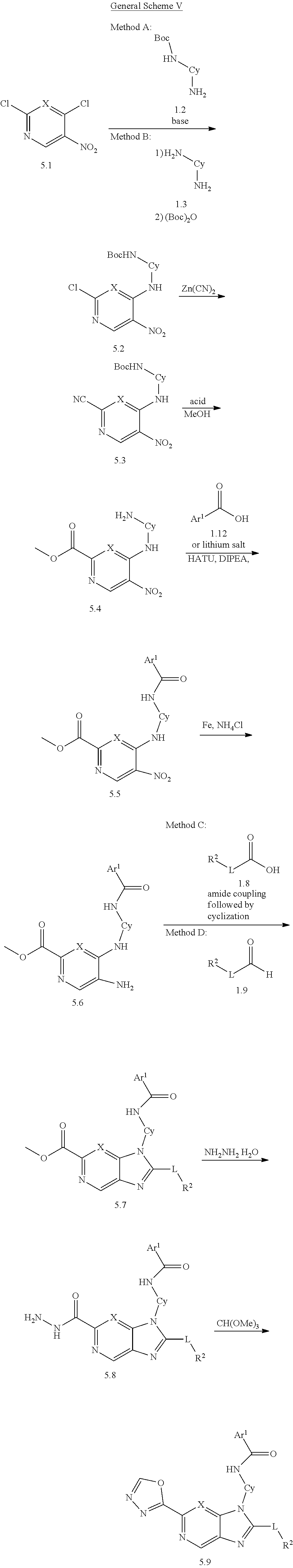

Compounds of formula 5.9 were obtained through the scheme depicted as General Scheme V. Beginning with 2,4-dichloro-5-nitropyridine of formula 5.1, a nucleophilic aromatic substitution reaction conducted under basic conditions was used to couple N-Boc-1,3-Cy-diamine compounds of formula 1.2 (Method A) to afford compounds of formula 5.2. Alternatively, reagent 5.1 underwent selective nucleophilic aromatic substitution by 1,3-Cy-diamine 1.3 (Method B), and the intermediate amine group of the resulting product was protected with a Boc-protecting group (or other suitable nitrogen protecting group) to afford compound 5.2. The 2-chloro group in compound 5.2 was converted to a cyano group using zinc cyanide to afford compound 5.3. The cyano group in compound 5.3 was then converted to a methyl ester via reaction with a strong acid such as HCl in the presence of methanol, and the amino Boc protecting group removed under acidic conditions to afford compound 5.4. Ar¹ groups were then installed by reacting an Ar¹ carboxylic acid of formula 1.12 or a basic salt (i.e., Li, K, or Na) thereof with the amine of formula 5.4 using acid coupling conditions known in the art, such as using one of the following reagents—HATU, EDCI, HOBt—along with a base, such as DIPEA (Hunig's base) or TEA to afford compounds of formula 5.5. The nitro group of a compound of formula 5.5 was reduced to an amino group affording pyridine-diamine compounds of formula 5.6. Subsequently, various L and R² groups were installed by coupling the pyridine-diamine compound 5.6 with acid 1.8 to form an amide intermediate (Method C), which was subsequently cyclized under either basic or acidic conditions to form compounds of formula 5.7. Alternatively, the pyridine-diamine compound of formula 5.6 was condensed with aldehyde 1.9 (Method D), followed by cyclization and in situ oxidation to afford compounds of formula 5.7. The methyl ester moiety in compound 5.7 was converted to a hydrazide using hydrazine hydrate to afford compounds of formula 5.8. The hydrazide moiety in compounds of formula 5.8 was converted to a 1,3,4-oxadiazole heteroaryl with trimethyl orthoformate to afford compounds of formula 5.9.

### Preparation of Example 1 Via General Scheme V (Methods A and C) (Relative Stereochemistry at Centers 1 and 3 of Cyclohexyl Ring in the Scheme Below is Cis)

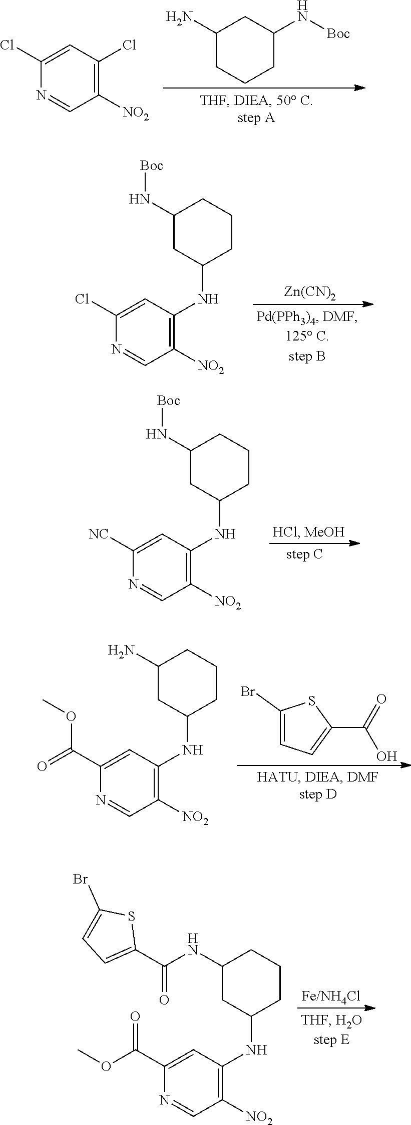

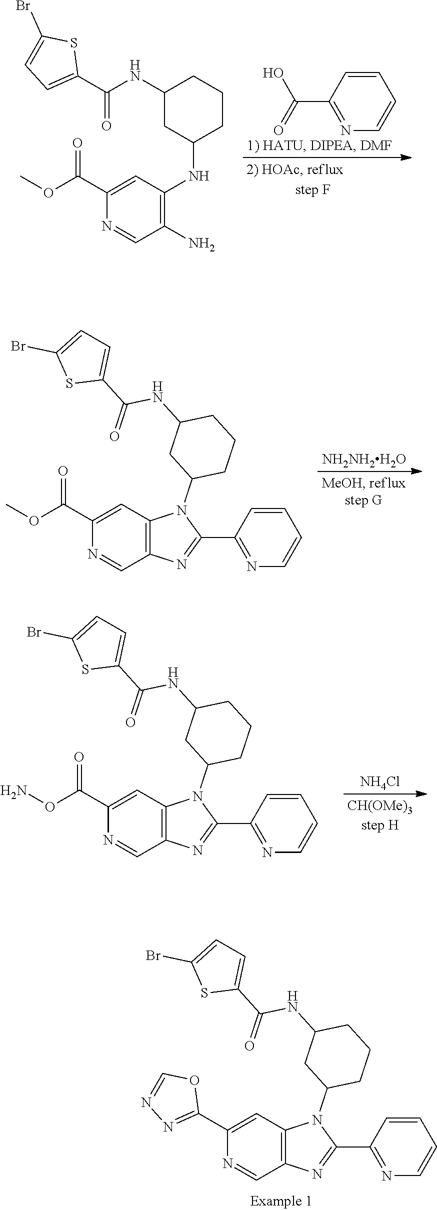

### Step A. Synthesis of tert-butyl (3-((2-chloro-5-nitropyridin-4-yl)amino)cyclohexyl)carbamate

To a solution of 2,4-dichloro-5-nitro-pyridine (500 mg, 2.59 mmol) in THF (10 mL) were added DIEA (670 mg, 5.18 mmol) and tert-butyl (3-aminocyclohexyl)carbamate (611 mg, 2.85 mmol). The reaction mixture was stirred at 50° C. for 5 hrs, and then concentrated to dryness. The residue was purified by flash silica gel chromatography to give tert-butyl (3-((2-chloro-5-nitropyridin-4-yl)amino)cyclohexyl)carbamate (920 mg, 91.8% yield). LC-MS: m/z 371.1 [M+H]⁺.

### Step B. Synthesis of tert-butyl (3-((2-cyano-5-nitropyridin-4-yl)amino)cyclohexyl)carbamate

A mixture of tert-butyl N-[3-[(2-chloro-5-nitro-4-pyridyl)amino]cyclohexyl]carbamate (300 mg, 809 μmol), Zn(CN)₂ (950 mg, 8.09 mmol) and Pd(PPh₃)₄ (187 mg, 162 μmol) in DMF (5 mL) was degassed and purged with N₂ 3 times. The reaction mixture was stirred at 125° C. for 15 hrs under N₂ atmosphere, then allowed to cool. The reaction mixture was filtered and concentrated under reduced pressure to give a residue, which was purified by silica gel chromatography to give tert-butyl (3-((2-cyano-5-nitropyridin-4-yl)amino)cyclohexyl)carbamate (190 mg, 63.7% yield). LC-MS: m/z 362.2 [M+H]⁺

### Step C. Synthesis of methyl 4-((3-aminocyclohexyl)amino)-5-nitropicolinate

A mixture of tert-butyl (3-((2-cyano-5-nitropyridin-4-yl)amino)cyclohexyl)carbamate (190 mg, 526 μmol) in HCl/MeOH (5 mL) was stirred at 70° C. for 15 hrs, and then concentrated to dryness to give methyl 4-((3-aminocyclohexyl)amino)-5-nitropicolinate (170 mg, HCl salt), which was used without further purification. LC-MS: m/z 295.1 [M+H]⁺

### Step D. Synthesis of methyl 4-((3-(5-bromothiophene-2-carboxamido)cyclohexyl)amino)-5-nitropicolinate

To a solution of methyl 4-((3-aminocyclohexyl)amino)-5-nitropicolinate (170 mg, 514 μmol, HCl salt) and 5-bromothiophene-2-carboxylic acid (128 mg, 617 μmol) in DMF (5 mL) were added HATU (293 mg, 771 μmol) and DIEA (199 mg, 1.54 mmol). The reaction mixture was stirred at rt for 15 hrs, quenched by the addition of brine (10 mL), and the aqueous portion extracted with EtOAc (50 mL×3). The combined organic layers were washed with brine (25 mL), dried over Na₂SO₄, filtered, and concentrated to dryness. The residue was purified by silica gel chromatography to give methyl 4-((3-(5-bromothiophene-2-carboxamido)cyclohexyl)amino)-5-nitropicolinate (170 mg, 65.7% yield). LC-MS: m/z 484.9 [M+H]⁺

### Step E. Synthesis of methyl 5-amino-4-((3-(5-bromothiophene-2-carboxamido)cyclohexyl)amino)picolinate

To a solution of methyl 4-((3-(5-bromothiophene-2-carboxamido)cyclohexyl)amino)-5-nitropicolinate (100 mg, 207 μmol) in THF (4 mL) were added Fe (57.8 mg, 1.03 mmol), H₂O (1 mL), and NH₄Cl (16.6 mg, 310 μmol). The reaction mixture was stirred at 75° C. for 5 hrs, allowed to cool, filtered, and concentrated to dryness to give methyl 5-amino-4-((3-(5-bromothiophene-2-carboxamido)cyclohexyl)amino)picolinate (100 mg), which was used without further purification. LC-MS: m/z 455.0 [M+H]⁺

### Step F. Synthesis of methyl 1-(3-(5-bromothiophene-2-carboxamido)cyclohexyl)-2-(pyridin-2-yl)-1H-imidazo[4,5-c]pyridine-6-carboxylate

To a solution of methyl 5-amino-4-((3-(5-bromothiophene-2-carboxamido)cyclohexyl)amino)picolinate (40 mg, 88 μmol) and pyridine-2-carboxylic acid (13.0 mg, 106 μmol) in DMF (2 mL) were added HATU (50.3 mg, 132 μmol) and DIEA (34.2 mg, 265 μmol). The reaction mixture was stirred at rt for 15 hrs, and then concentrated to dryness to give methyl 5-amino-4-((3-(5-bromothiophene-2-carboxamido)cyclohexyl)amino)picolinate (100 mg), which was used without further purification. LC-MS: m/z 560.0 [M+H]⁺

A solution of methyl 5-amino-4-((3-(5-bromothiophene-2-carboxamido)cyclohexyl)amino)picolinate (400 mg, 358 μmol, 50% purity) in AcOH (1 mL) was stirred at 100° C. for 20 hrs. The reaction mixture was concentrated under reduced pressure to give a residue, which was purified by silica gel chromatography to give methyl 1-(3-(5-bromothiophene-2-carboxamido)cyclohexyl)-2-(pyridin-2-yl)-1H-imidazo[4,5-c]pyridine-6-carboxylate (190 mg, 83.4% yield). LC-MS: m/z 542.0 [M+H]⁺

### Step G. Synthesis of 5-bromo-N-(3-(6-(hydrazinecarbonyl)-2-(pyridin-2-yl)-1H-imidazo[4,5-c]pyridin-1-yl)cyclohexyl)thiophene-2-carboxamide

To a solution of methyl 1-(3-(5-bromothiophene-2-carboxamido)cyclohexyl)-2-(pyridin-2-yl)-1H-imidazo[4,5-c]pyridine-6-carboxylate (25 mg, 46 μmol) in MeOH (1 mL) was added NH₂NH₂·H₂O (13.6 mg, 231 μmol, 13.2 μL, 85% purity). The reaction mixture was stirred at 70° C. for 1 hr, and then concentrated to dryness to give 5-bromo-N-(3-(6-(hydrazinecarbonyl)-2-(pyridin-2-yl)-1H-imidazo[4,5-c]pyridin-1-yl)cyclohexyl)thiophene-2-carboxamide (26 mg), which was used without further purification. LC-MS: m/z 542.0 [M+H]⁺

### Step H. Synthesis of N-(3-(6-(1,3,4-oxadiazol-2-yl)-2-(pyridin-2-yl)-1H-imidazo[4,5-c]pyridin-1-yl)cyclohexyl)-5-bromothiophene-2-carboxamide (Example 1)

To a solution of 5-bromo-N-(3-(6-(hydrazinecarbonyl)-2-(pyridin-2-yl)-1H-imidazo[4,5-c]pyridin-1-yl)cyclohexyl)thiophene-2-carboxamide (26 mg, 48 μmol) in CH(OEt)₃ (1 mL) was added NH₄Cl (7.72 mg, 144 μmol). The reaction mixture was stirred at 100° C. for 20 hrs, and then concentrated under reduced pressure to a residue, which was purified by prep-HPLC to give Example 1 (relative stereochemistry at centers 1 and 3 of cyclohexyl ring is cis) (3.6 mg, 12.9% yield). ¹H NMR (400 MHz, CDCl₃) δ 9.23 (s, 1H), 8.75 (d, 1H), 8.60 (s, 2H), 8.31-8.28 (m, 1H), 7.94-7.90 (m, 1H), 7.48-7.44 (m, 1H), 7.00 (d, 1H), 76.12 (d, 1H), 5.86 (br s, 1H), 4.18 (br s, 1H), 2.51-2.48 (m, 1H), 2.33-2.16 (m, 4H), 2.07-2.03 (m, 1H), 1.70 (br s, 2H), 1.50-1.40 (m, 1H). LC-MS: m/z 552.2 [M+H]⁺.

### Preparation of Example 380

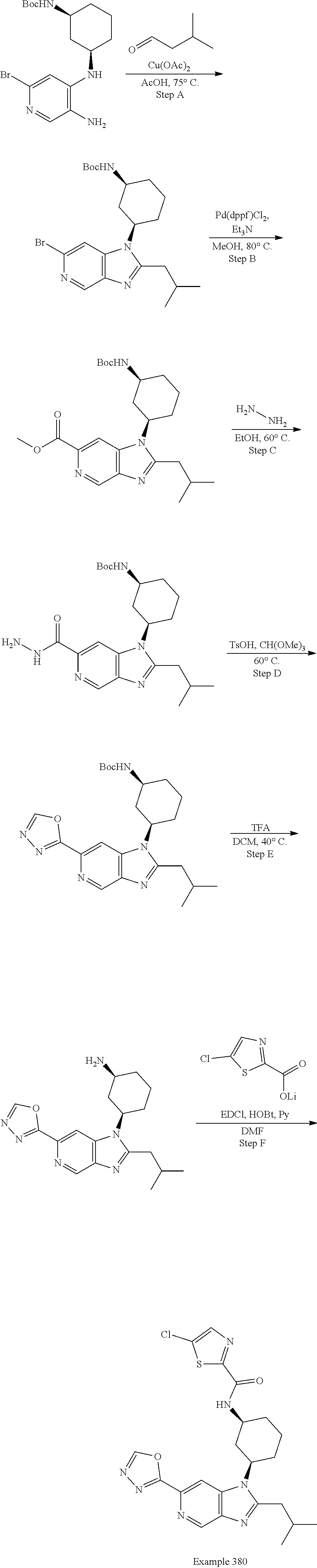

### Step A. Synthesis of tert-butyl ((1S,3R)-3-(6-bromo-2-isobutyl-1H-imidazo[4,5-c]pyridin-1-yl)cyclohexyl)carbamate

To a solution of tert-butyl ((1S,3R)-3-((5-amino-2-bromopyridin-4-yl)amino)cyclohexyl)carbamate (1 g, 2.60 mmol) in AcOH (10 mL) was added copper(II) acetate monohydrate (518 mg, 2.60 mmol) and 3-methylbutanal (268 mg, 3.11 mmol). The reaction mixture was stirred at 75° C. for 16 hrs and then concentrated to dryness. Water (15 mL) was added to the residue, and the aqueous portion was extracted with EtOAc (30 mL×3). The combined organic layers were washed with brine (10 mL), dried over Na₂SO₄, filtered, and concentrated to dryness to afford tert-butyl ((1S,3R)-3-(6-bromo-2-isobutyl-1H-imidazo[4,5-c]pyridin-1-yl)cyclohexyl)carbamate (320 mg, 27% yield). LC-MS: m/z 451 [M+H]⁺.

### Step B. Synthesis of tert-butyl ((1S,3R)-3-(6-(hydrazinecarbonyl)-2-isobutyl-1H-imidazo[4,5-c]pyridin-1-yl)cyclohexyl)carbamate

To a solution of tert-butyl ((1S,3R)-3-(6-bromo-2-isobutyl-1H-imidazo[4,5-c]pyridin-1-yl)cyclohexyl)carbamate (500 mg, 1.11 mmol) in MeOH (15 mL) were added Pd (dppf)Cl₂ (81.0 mg, 111 μmol) and TEA (336 mg, 3.32 mmol). The reaction mixture was degassed under vacuum and purged with CO (31.0 mg, 1.11 mmol) several times, then stirred at 80° C. for 16 hrs under CO atmosphere. The reaction mixture was cooled to rt and filtered through a celite pad. Water (20 mL) was added to the filtrate, and the aqueous portion extracted with EtOAc (20 mL×3). The combined organic layers were washed with brine (20 mL), dried over Na₂SO₄, filtered, and concentrated to dryness. The residue was purified by flash column chromatography on silica gel to afford tert-butyl ((1S,3R)-3-(6-(hydrazinecarbonyl)-2-isobutyl-1H-imidazo[4,5-c]pyridin-1-yl)cyclohexyl)carbamate (330 mg, 69% yield). LC-MS: m/z 431 [M+H]⁺.

### Step C. Synthesis of tert-butyl ((1S,3R)-3-(6-(hydrazinecarbonyl)-2-isobutyl-1H-imidazo[4,5-c]pyridin-1-yl)cyclohexyl)carbamate

To a solution of methyl 1-((1R,3S)-3-((tert-butoxycarbonyl)amino)cyclohexyl)-2-isobutyl-1H-imidazo[4,5-c]pyridine-6-carboxylate (300 mg, 697 μmol) in EtOH (10 mL) was added hydrazine (558 mg, 6.97 mmol, 40 wt %). The reaction mixture was stirred at 60° C. for 3 hrs and then concentrated to dryness. The residue was purified by prep-HPLC to afford tert-butyl ((1S,3R)-3-(6-(hydrazinecarbonyl)-2-isobutyl-1H-imidazo[4,5-c]pyridin-1-yl)cyclohexyl)carbamate (170 mg, 57% yield). LC-MS: m/z 431 [M+H]⁺.

### Step D. Synthesis of tert-butyl ((1S,3R)-3-(2-isobutyl-6-(1,3,4-oxadiazol-2-yl)-1H-imidazo[4,5-c]pyridin-1-yl)cyclohexyl)carbamate

To a solution of tert-butyl ((1S,3R)-3-(6-(hydrazinecarbonyl)-2-isobutyl-1H-imidazo[4,5-c]pyridin-1-yl)cyclohexyl)carbamate (160 mg, 372 μmol) in trimethyl ortho formate (1 mL) was added p-toluenesulfonic acid (7 mg, 37.2 μmol). The reaction mixture was stirred at 60° C. for 16 hrs and then concentrated to dryness. The residue was purified by prep-HPLC to afford tert-butyl ((1S,3R)-3-(2-isobutyl-6-(1,3,4-oxadiazol-2-yl)-1H-imidazo[4,5-c]pyridin-1-yl)cyclohexyl)carbamate (100 mg, 61% yield). LC-MS: m/z 441 [M+H]⁺.

### Step E. Synthesis of (1S,3R)-3-(2-isobutyl-6-(1,3,4-oxadiazol-2-yl)-1H-imidazo[4,5-c]pyridin-1-yl)cyclohexan-1-amine

To a solution of tert-butyl ((1S,3R)-3-(2-isobutyl-6-(1,3,4-oxadiazol-2-yl)-1H-imidazo[4,5-c]pyridin-1-yl)cyclohexyl)carbamate (10 mg, 23 μmol) in DCM (2 mL) was added TFA (0.2 mL). The reaction mixture was stirred at 40° C. for 2 hrs and then concentrated to dryness to afford (1S,3R)-3-(2-isobutyl-6-(1,3,4-oxadiazol-2-yl)-1H-imidazo[4,5-c]pyridin-1-yl)cyclohexan-1-amine (70 mg). LC-MS: m/z 341 [M+H]⁺.

### Step F. Synthesis of 5-chloro-N-((1S,3R)-3-(2-isobutyl-6-(1,3,4-oxadiazol-2-yl)-1H-imidazo[4,5-c]pyridin-1-yl)cyclohexyl)thiazole-2-carboxamide (Example 380)

To a solution of (1S,3R)-3-(2-isobutyl-6-(1,3,4-oxadiazol-2-yl)-1H-imidazo[4,5-c]pyridin-1-yl)cyclohexan-1-amine (120 mg, 352 μmol) and lithium 5-chlorothiazole-2-carboxylate (90 mg, 529 μmol) in DMF (3 mL) were added EDCI (135 mg, 705 μmol), HOBt (108 mg, 705 μmol) and pyridine (223 mg, 2.82 mmol). The reaction mixture was stirred at rt for 16 hrs. Water (20 mL) was added to the reaction mixture, and the aqueous portion extracted with EtOAc (20 mL×3). The combined organic layers were washed with brine (20 mL), dried over Na₂SO₄, filtered, and concentrated to dryness. The residue was purified by prep-HPLC to afford Example 380 (16 mg, 10% yield). ¹H NMR (400 MHz, DMSO-d₆) δ 9.41 (s, 1H), 9.05-9.00 (m, 2H), 8.48 (s, 1H), 8.06 (s, 1H), 4.79-4.66 (m, 1H), 4.28-4.14 (m, 1H), 2.93 (d, 2H), 2.65-2.55 (m, 1H), 2.31-2.19 (m, 1H), 2.11-1.81 (m, 5H), 1.72-1.57 (m, 2H), 1.03-1.00 (m, 6H); LC-MS: m/z 486.1 [M+H]⁺.

General Scheme V set forth above was used to synthesize the following compounds by using appropriate starting materials:

| Ex. | | | LC-MS: |
|---|---|---|---|
| No. | Structure | ¹H NMR | m/z |
| 2 | 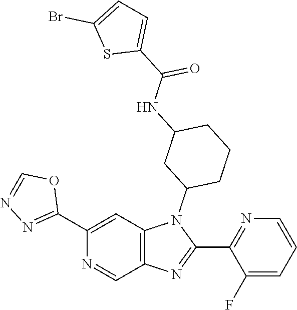 [SMILES: O=C(NC1CCCC(n2c(-c3ncccc3F)nc3cnc(-c4nnco4)cc32)C1)c1ccc(Br)s1] | (400 MHz, CDCl₃) δ 9.27 (s, 1 H), 8.68-8.51 (m, 3 H), 7.78-7.62 (m, 1 H), 7.60- 7.47 (m, 1 H), 7.30-7.23 (m, 2 H), 6.99 (d, 1 H), 6.47- 6.18 (m, 1 H), 4.92-4.76 (m, 1 H), 4.16-4.01 (m, 1 H), 2.49 (br d, 1 H), 2.36-2.09 (m, 4 H), 2.07-1.97 (m, 1 H), 1.77- 1.70 (m, 1 H), 1.57-1.39 (m, 1 H). | 570.2 [M + H]⁺ |
| | (relative | | |
| | stereochemistry at | | |
| | centers 1 and 3 of | | |
| | cyclohexyl ring is cis) | | |

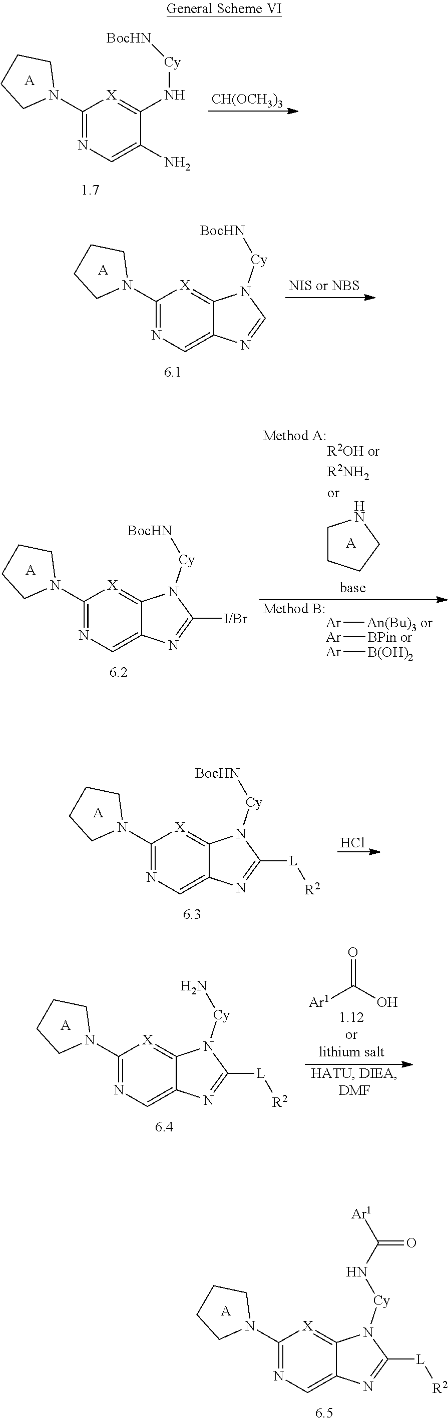

Compounds of formula 6.5 were obtained through the scheme depicted as General Scheme VI. The pyridine-diamine compound of formula 1.7 (made in accordance with General Scheme I) was reacted with trimethyl orthoformate to form the imidazole of azabenzimidazole compound of formula 6.1. An iodide or bromide was installed at the 2-position of the azabenzimidazole compound of formula 6.1 using a bromination or iodination reagent to afford compounds of formula 6.2. Subsequently, various L and R² groups were installed by displacing the halide in compounds of formula 6.2 with an R²-alcohol or R²-primary amine or heterocyclic amine (saturated or aromatic) (Method A) to afford compounds of formula 6.3. Alternatively, using Method B, the halide in compound of formula 6.2 was converted to an aryl or R¹ heteroaryl group using a Stille coupling (R¹-SnBu₃) or a Suzuki coupling (R¹-BPin or R¹—B(OH)₂) to afford compounds of formula 6.3. The amino Boc protecting group in compounds of formula 6.3 was removed under acidic conditions to afford compounds of formula 6.4. Ar¹ groups were then installed by using an amide coupling with the carboxylic acid 1.12 or its lithium salt to form compounds of formula 6.5.

### Preparation of Example 63 Via General Scheme VI (Method A)

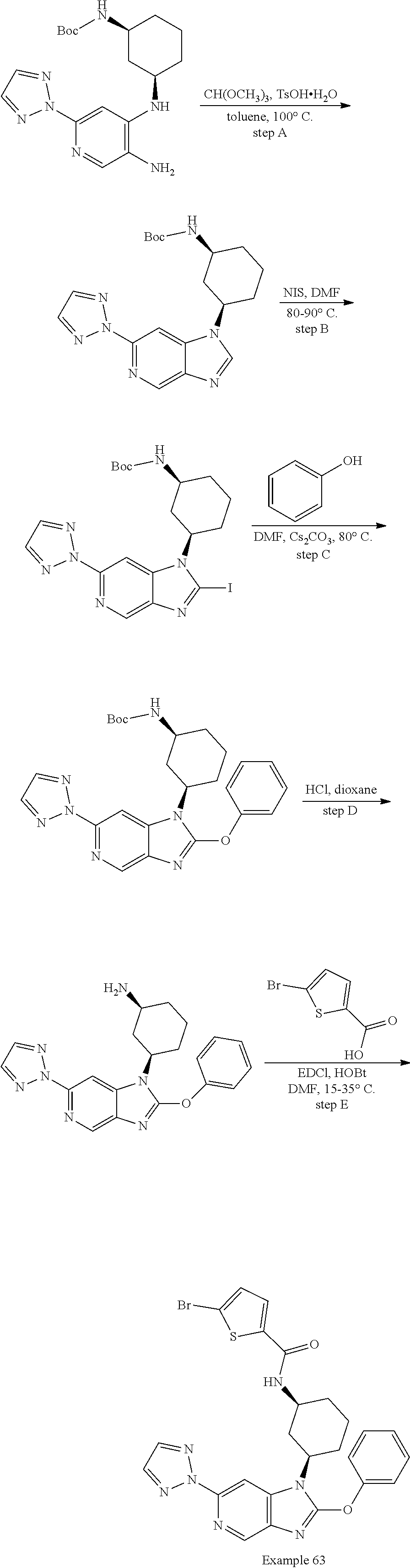

### Step A. Synthesis of tert-butyl (1S,3R)-3-(6-(2H-1,2,3-triazol-2-yl)-1H-imidazo[4,5-c]pyridin-1-yl)cyclohexyl)carbamate

A reaction mixture of tert-butyl ((1S,3R)-3-((5-amino-2-(2H-1,2,3-triazol-2-yl)pyridin-4-yl)amino)cyclohexyl)carbamate (1.5 g, 4.0 mmol), trimethoxymethane (4.26 g, 40.2 mmol, 4.40 mL) and TsOH·H₂O (76.4 mg, 402 μmol) in toluene (40 mL) was stirred at 100° C. for 18 hrs, and then concentrated to dryness. The residue was purified by flash silica gel chromatography to afford tert-butyl ((1S,3R)-3-(6-(2H-1,2,3-triazol-2-yl)-1H-imidazo[4,5-c]pyridin-1-yl)cyclohexyl)carbamate (950 mg, 61.7% yield). LC-MS: m/z 384.1 [M+H]⁺

### Step B. Synthesis of tert-butyl ((1S,3R)-3-(2-iodo-6-(2H-1,2,3-triazol-2-yl)-1H-imidazo[4,5-c]pyridin-1-yl)cyclohexyl)carbamate

To a solution of tert-butyl ((1S,3R)-3-(6-(2H-1,2,3-triazol-2-yl)-1H-imidazo[4,5-c]pyridin-1-yl)cyclohexyl)carbamate (950 mg, 2.48 mmol) in DMF (15 mL) was added NIS (2.79 g, 12.4 mmol) in portions over 5 hrs at 80° C., and the reaction mixture was stirred at 90° C. for 18 hrs under N₂, then allowed to cool. The reaction mixture was poured into 50 mL of water and the aqueous portion extracted with EtOAc (30 mL×3). The combined organic layers were washed with sat. aq. Na₂SO₃ solution (30 mL×2), brine (30 mL), dried over Na₂SO₄, and concentrated to dryness. The residue was purified by flash silica gel chromatography to afford tert-butyl ((1S,3R)-3-(2-iodo-6-(2H-1,2,3-triazol-2-yl)-1H-imidazo[4,5-c]pyridin-1-yl)cyclohexyl)carbamate (940 mg, 74.5% yield). ¹H NMR (400 MHz, CDCl₃) δ 8.96 (s, 1H), 8.18 (s, 1H), 7.92 (s, 2H), 4.65-4.51 (m, 2H), 3.75 (br s, 1H), 2.78 (s, 1H), 2.42 (m, 1H), 2.23-2.06 (m, 4H), 2.00 (m, 1H), 1.44 (s, 9H), 1.37-1.31 (m, 1H); LC-MS: m/z 510.0 [M+H]⁺.

### Step C. Synthesis of tert-butyl ((1S,3R)-3-(2-phenoxy-6-(2H-1,2,3-triazol-2-yl)-1H-imidazo[4,5-c]pyridin-1-yl)cyclohexyl)carbamate

A reaction mixture of tert-butyl ((1S,3R)-3-(2-iodo-6-(2H-1,2,3-triazol-2-yl)-1H-imidazo[4,5-c]pyridin-1-yl)cyclohexyl)carbamate (80 mg, 157 μmol), phenol (22.2 mg, 236 μmol), and Cs₂CO₃ (102 mg, 314 μmol) in MeCN (2 mL) was stirred at 80° C. for 18 hrs. The reaction mixture was concentrated to dryness. The residue was purified by flash silica gel chromatography to afford tert-butyl ((1S,3R)-3-(2-phenoxy-6-(2H-1,2,3-triazol-2-yl)-1H-imidazo[4,5-c]pyridin-1-yl)cyclohexyl)carbamate (60 mg, 80.3% yield). LC-MS: m/z 476.6 [M+H]⁺.

### Step D. Synthesis of (1S,3R)-3-(2-phenoxy-6-(2H-1,2,3-triazol-2-yl)-1H-imidazo[4,5-c]pyridin-1-yl)cyclohexanamine

To a solution of tert-butyl ((1S,3R)-3-(2-phenoxy-6-(2H-1,2,3-triazol-2-yl)-1H-imidazo[4,5-c]pyridin-1-yl)cyclohexyl)carbamate (50 mg, 111 μmol) in DCM (2 mL) was added TFA (1.23 g, 10.8 mmol). The reaction mixture was stirred at rt for 18 hrs and then concentrated to dryness to afford (1S,3R)-3-(2-phenoxy-6-(2H-1,2,3-triazol-2-yl)-1H-imidazo[4,5-c]pyridin-1-yl)cyclohexanamine, which was used without further purification. LC-MS: m/z 376.2 [M+H]⁺.

### Step E. Synthesis of 5-bromo-N-((1S,3R)-3-(2-phenoxy-6-(2H-1,2,3-triazol-2-yl)-1H-imidazo[4,5-c]pyridin-1-yl)cyclohexyl)thiophene-2-carboxamide (Example 63)

A reaction mixture of (1S,3R)-3-(2-phenoxy-6-(2H-1,2,3-triazol-2-yl)-1H-imidazo[4,5-c]pyridin-1-yl)cyclohexanamine (50 mg, 102 μmol TFA salt), 5-bromothiophene-2-carboxylic acid (25.4 mg, 123 μmol), EDCI (29.4 mg, 153 μmol), DIEA (39.6 mg, 307 μmol, 53.4 μL), and HOBt (20.7 mg, 153 μmol) in DMF (2 mL) was stirred at rt for 18 hrs. The reaction mixture was diluted with MeOH (2 mL) and purified by prep-HPLC (column: Boston Prime C18 150*30 mm*5 um; mobile phase: [water (0.05% NH₃H₂O+10 mM NH₄HCO₃)-ACN]; B %: 45%-75%, 10 min) to afford Example 63 (24 mg, 41.6% yield). ¹H NMR (400 MHz, CDCl₃) δ 8.73 (br s, 1H), 7.99 (s, 1H), 7.94 (s, 2H), 7.55-7.48 (m, 2H), 7.42-7.29 (m, 4H), 7.05-7.01 (m, 1H), 6.17 (m, 1H), 4.71-4.60 (m, 1H), 4.33-4.17 (m, 1H), 2.50 (m, 1H), 2.28-2.10 (m, 5H), 1.75-1.66 (m, 1H), 1.54-1.38 (m, 1H); LC-MS: m/z 564.2 [M+H]⁺.

General Scheme VI set forth above was used to synthesize the following compounds by using appropriate starting materials:

| Ex. | | | LC-MS: |
|---|---|---|---|
| No. | Structure | ¹H NMR | m/z |
| 56 | 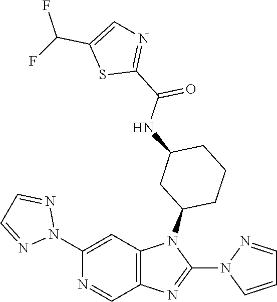 [SMILES: O=C(N[C@H]1CCC[C@@H](n2c(-n3cccn3)nc3cnc(-n4nccn4)cc32)C1)c1ncc(C(F)F)s1] | (400 MHz, CDCl₃) δ 9.00 (s, 1 H), 8.38 (d, 1 H), 8.26 (s, 1 H), 7.99 (t, 1 H), 7.96 (s, 2 H), 7.91 (d, 1 H), 7.33-7.29 (m, 1 H), 7.08-6.77 (m, 1 H), 6.62 (dd, 1 H), 5.63-5.50 (m, 1 H), 4.27-4.15 (m, 1 H), 2.57 (m, 1 H), 2.42-2.04 (m, 6 H), 1.60-1.49 (m, 1 H). | 511.1 [M + H]⁺ |
| 57 | 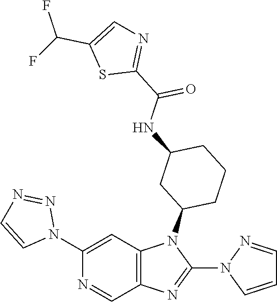 [SMILES: O=C(N[C@H]1CCC[C@@H](n2c(-n3cccn3)nc3cnc(-n4ccnn4)cc32)C1)c1ncc(C(F)F)s1] | (400 MHz, CDCl₃) δ 8.89 (s, 1 H), 8.69 (d, 1 H), 8.44 (s, 1 H), 8.38 (d, 1 H), 8.00 (s, 1 H), 7.93-7.88 (m, 2 H), 7.36-7.30 (m, 1 H), 7.08-6.77 (m, 1 H), 6.63 (m, 1 H), 5.65-5.50 (m, 1 H), 4.27-4.15 (m, 1 H), 2.56 (br s, 1 H), 2.41-2.03 (m, 6 H), 1.59-1.52 (m, 1 H). | 511.1 [M + H]⁺ |
| 67 | 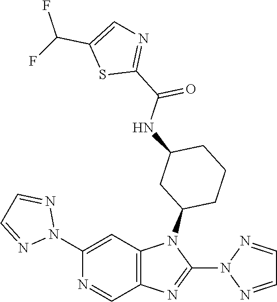 [SMILES: O=C(N[C@H]1CCC[C@@H](n2c(-n3nccn3)nc3cnc(-n4nccn4)cc32)C1)c1ncc(C(F)F)s1] | (400 MHz, CDCl₃) δ 9.10 (s, 1 H), 8.29 (s, 1 H), 8.10 (s, 2 H), 7.99-7.94 (m, 3 H), 7.30- 7.27 (m, 1 H), 7.29 (br s, 1 H), 7.07-6.75 (m, 1 H), 5.12-4.99 (m, 1 H), 4.23-4.12 (m, 1 H), 2.59 (br d, 1 H), 2.37-2.11 (m, 5 H), 1.64 (br s, 1 H), 1.55-1.48 (m, 1 H). | 512.2 [M + H]⁺ |
| 69 | 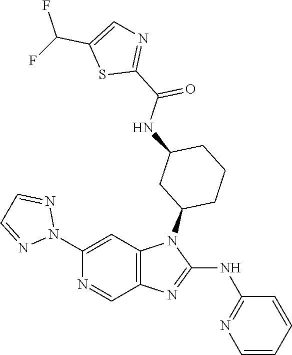 [SMILES: O=C(N[C@H]1CCC[C@@H](n2c(Nc3ccccn3)nc3cnc(-n4nccn4)cc32)C1)c1ncc(C(F)F)s1] | (400 MHz, CDCl₃) δ 8.65 (s, 1 H), 8.01-7.96 (m, 2 H), 7.92 (s, 2 H), 7.81 (br s, 1 H), 7.65-7.58 (m, 1 H), 7.10 (br s, 1 H), 7.07- 6.78 (m, 1 H), 6.67 (br s, 1 H), 5.07 (br s, 1 H), 4.29 (m, 1 H), 2.44-2.31 (m, 3 H), 2.25 (m, 1 H), 2.15-2.01 (m, 2 H), 1.79-1.70 (m, 1 H), 1.55-1.43 (m, 1 H). | 537.3 [M + H]⁺ |
| 71 | 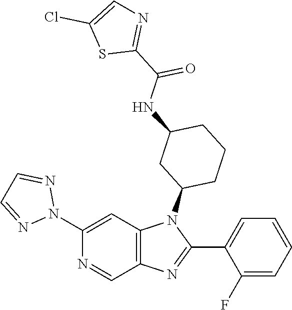 [SMILES: O=C(N[C@H]1CCC[C@@H](n2c(-c3ccccc3F)nc3cnc(-n4nccn4)cc32)C1)c1ncc(Cl)s1] | (400 MHz, CDCl₃) δ 9.11 (s, 1H), 8.28 (s, 1H), 7.97 (s, 2H), 7.72-7.64 (m, 3 H), 7.44- 7.41 (m, 1 H), 7.36-7.31 (m, 1 H), 7.14 (d, 1 H), 4.32-4.26 (m, 1 H), 4.09-4.05 (m, 1 H), 2.46-2.43 (m, 1 H), 2.35-2.26 (m, 2 H), 2.19-2.08 (m, 3 H), 1.54-1.49 (m, 2H). | 523.2 [M + H]⁺ |
| 73 | 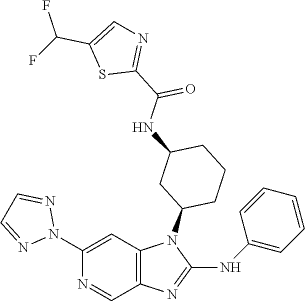 [SMILES: O=C(N[C@H]1CCC[C@@H](n2c(Nc3ccccc3)nc3cnc(-n4nccn4)cc32)C1)c1ncc(C(F)F)s1] | (400 MHz, CDCl₃) δ 8.74 (s, 1 H), 8.01-7.97 (m, 2 H), 7.90 (s, 2 H), 7.61 (br d, 2 H), 7.39-7.33 (m, 3 H), 7.10 (t, 1 H), 7.06-6.78 (m, 1 H), 4.40 (br t, 1 H), 4.13 (br d, 1 H), 2.50 (br d, 1 H), 2.28-2.05 (m, 5 H), 1.59- 1.50 (m, 2 H), 1.32-1.23 (m, 1 H). | 536.2 [M + H]⁺ |
| 79 | 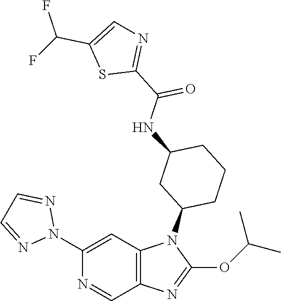 [SMILES: CC(C)Oc1nc2cnc(-n3nccn3)cc2n1[C@@H]1CCC[C@H](NC(=O)c2ncc(C(F)F)s2)C1] | (400 MHz, CDCl₃) δ 8.72 (br s, 1 H), 7.98 (s, 1 H), 7.95-7.88 (m, 2 H), 7.29-7.23 (m, 1 H), 7.07-6.75 (m, 1 H), 5.43 (spt, 1 H), 4.49- 4.35 (m, 1 H), 4.19 (tdt, 1 H), 2.38 (br d, 1 H), 2.24-1.93 (m, 5 H), 1.66-1.59 (m, 1 H), 1.53 (d, 6 H), 1.46 (br dd, 1 H). | 503.3 [M + H]⁺ |
| 97 | 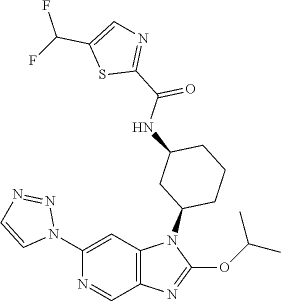 [SMILES: CC(C)Oc1nc2cnc(-n3ccnn3)cc2n1[C@@H]1CCC[C@H](NC(=O)c2ncc(C(F)F)s2)C1] | (400 MHz, CDCl₃) δ 8.61 (d, 2 H), 8.13 (s, 1 H), 7.99 (t, 1 H), 7.85 (s, 1 H), 7.30 (br d, 1 H), 7.08-6.77 (m, 1 H), 5.43 (brt, 1 H), 4.45 (tt, 1 H), 4.20 (tdt, 1 H), 2.40 (br d, 1 H), 2.25-1.95 (m, 5 H), 1.61 (br s, 1 H), 1.54 (dd, 6 H), 1.51-1.45 (m, 1 H). | 503.3 [M + H]⁺ |
| 98 | 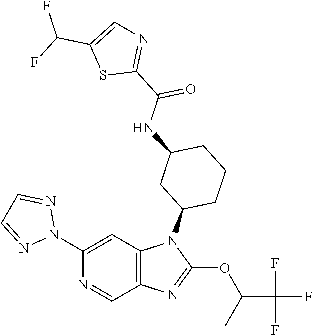 [SMILES: CC(Oc1nc2cnc(-n3nccn3)cc2n1[C@@H]1CCC[C@H](NC(=O)c2ncc(C(F)F)s2)C1)C(F)(F)F] | (400 MHz, CDCl₃) δ 8.75 (br s, 1 H), 8.10- 7.87 (m, 4 H), 7.37-7.22 (m, 2 H), 7.11-6.78 (m, 1 H), 5.76 (br d, 1 H), 4.44 (br s, 1 H), 4.21 (br s, 1 H), 2.45 (br s, 1 H), 2.25-2.10 (m, 5 H), 1.64 (br s, 4 H), 1.47 (br s, 1 H). | 557.2 [M + H]⁺ |
| 101 | 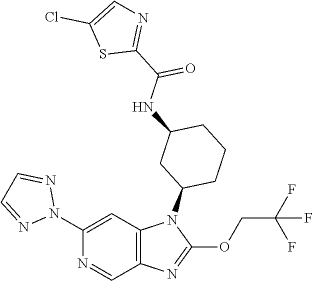 [SMILES: O=C(N[C@H]1CCC[C@@H](n2c(OCC(F)(F)F)nc3cnc(-n4nccn4)cc32)C1)c1ncc(Cl)s1] | (400 MHz, CDCl₃) δ 8.68 (d, 1 H), 7.94 (d, 1 H), 7.84 (s, 2 H), 7.57 (s, 1 H), 7.20 (s, 1 H), 7.04 (br d, 1 H), 4.95 (q, 2 H), 4.36 (dt, 1 H), 4.10 (tdt, 1 H), 2.36 (br d, 1 H), 2.15- 1.94 (m, 6 H), 1.43-1.34 (m, 1 H). | 527.1 [M + H]⁺ |

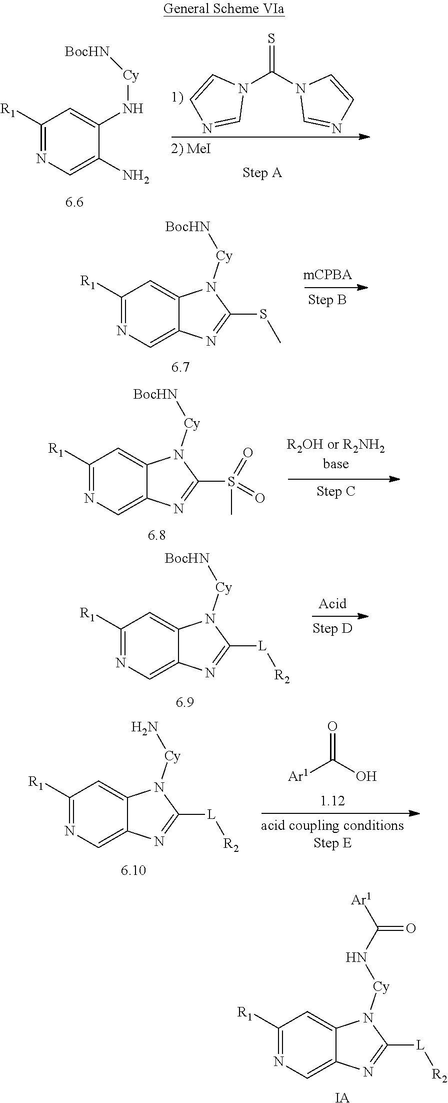

Compounds of formula IA wherein L is —O— or —NH— were obtained through the scheme depicted as General Scheme VIa. The pyridine-diamine compound of formula 6.6 (made in accordance with General Schemes I or IA) was first reacted with thiocarbonyliimidazole, and then methyl iodide to afford compounds of formula 6.7. The 2-methylthio moiety of formula 6.7 was oxidized to afford compounds of formula 6.8. The methylsulfonyl moiety of formula 6.8 was then displaced by an R²-alcohol or R²-primary amine (wherein R² is a heteroaryl or heterocycloalkyl) in the presence of a base such as cesium carbonate to afford compounds of formula 6.9. The amino Boc protecting group in compounds of formula 6.9 was removed under acidic conditions to afford compounds of formula 6.10. Ar¹ groups were then installed by reacting an Ar¹ carboxylic acid of formula 1.12 or a basic salt (i.e., Li, K, or Na) thereof with the amine of formula 6.10 using acid coupling conditions known in the art, such as using one of the following reagents—HATU, EDCI, HOBt—along with a base, such as DIPEA (Hunig's base) or TEA to afford compounds of formula IA wherein L is —O— or —NH—.

Alternatively, Steps A, B, and C of General Scheme VIa can be carried out starting from compounds of formula 3.1:

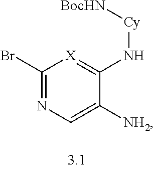

prepared in accordance with General Scheme III, and then the R¹ group is installed using Methods A, B, C, D, E, F, and G from General Scheme IV, before Steps D and E of General Scheme VIa are carried out.

### Preparation of Example 191 Via General Scheme VIa

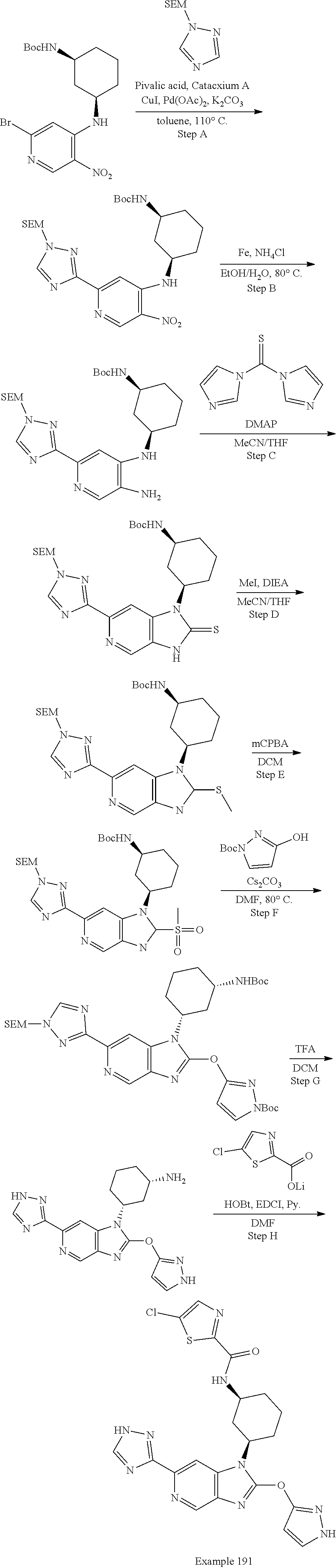

### Step A. tert-butyl ((1S,3R)-3-((5-nitro-2-(1-((2-(trimethylsilyl)ethoxy)methyl)-1H-1,2,4-triazol-3-yl)pyridin-4-yl)amino)cyclohexyl)carbamate

To a solution of tert-butyl ((1S,3R)-3-((2-bromo-5-nitropyridin-4-yl)amino)cyclohexyl)carbamate (2 g, 5 mmol), 1-((2-(trimethylsilyl)ethoxy)methyl)-1H-1,2,4-triazole (3 g, 17.5 mol, 3.5 eq.), pivalic acid (0.5 mL, 5 mmol), CuI (0.6 g, 3 mmol, 0.6 eq.) and K₂CO₃ (2 g, 0.01 mol, 3 eq.) in toluene (20 mL) were added Pd(OAc)₂ (0.4 g, 2 mmol, 0.4 eq.) and Catacxium A (1 g, 4 mmol, 0.8 eq.) at rt. The reaction mixture was stirred at 110° C. for 16 hrs under N₂. Water (20 mL) was added, and the aqueous portion extracted with EtOAc (20 mL×3). The combined organic layers were washed with brine (20 mL), dried over Na₂SO₄, filtered, and concentrated to dryness. The residue was purified by flash column chromatography on silica gel to afford tert-butyl ((1S,3R)-3-((5-nitro-2-(1-((2-(trimethylsilyl)ethoxy)methyl)-1H-1,2,4-triazol-3-yl)pyridin-4-yl)amino)cyclohexyl)carbamate (2 g, 80% yield). LC-MS. m/z 534 [M+H]⁺.

### Step B. Synthesis of tert-butyl ((1S,3R)-3-(2-(2-fluorophenyl)-6-(tetrahydrofuran-2-yl)-1H-imidazo[4,5-c]pyridin-1-yl)cyclohexyl)carbamate

To a solution of tert-butyl ((1S,3R)-3-((5-nitro-2-(1-((2-(trimethylsilyl)ethoxy)methyl)-1H-1,2,4-triazol-3-yl)pyridin-4-yl)amino)cyclohexyl)carbamate (2 g, 4 mmol) in a mixture of EtOH (15 mL) and water (5.0 mL) were added ammonium chloride (2 g, 40 mmol, 10 eq.) and iron (2 g, 40 mmol, 10 eq.). The reaction mixture was stirred at 80° C. for 2 hrs under N₂ atmosphere. The reaction mixture was filtered through Celite pad, and the filtrate was then concentrated under reduced pressure. Water (20 mL) was added to the residue, and the aqueous portion extracted with EtOAc (50 mL×3). The combined organic layers were washed with brine (20 mL), dried over Na₂SO₄, filtered, and concentrated to dryness. The residue was purified by flash column chromatography on silica gel to afford tert-butyl ((1S,3R)-3-(2-(2-fluorophenyl)-6-(tetrahydrofuran-2-yl)-1H-imidazo[4,5-c]pyridin-1-yl)cyclohexyl)carbamate (1.8 g, 90% yield). LC-MS: m/z 504 [M+H]⁺.

### Step C. Synthesis of tert-butyl ((1S,3R)-3-(2-thioxo-6-(1-((2-(trimethylsilyl)ethoxy)methyl)-1H-1,2,4-triazol-3-yl)-2,3-dihydro-1H-imidazo[4,5-c]pyridin-1-yl)cyclohexyl)carbamate

To a solution of tert-butyl ((1S,3R)-3-(2-(2-fluorophenyl)-6-(tetrahydrofuran-2-yl)-1H-imidazo[4,5-c]pyridin-1-yl)cyclohexyl)carbamate (1.0 g, 2.0 mmol) in MeCN/THF (10 mL, v/v=1:1) were added DMAP (0.2 g, 2 mmol) and di(1H-imidazol-1-yl)methanethione (0.4 g, 2.4 mmol, 1.2 eq.) at 0° C. The reaction mixture was warmed to rt and stirred for 2 hrs. Water (50 mL) was added, and the aqueous portion extracted with EtOAc (50 mL×3). The combined organic layers were washed with brine (50 mL), dried over Na₂SO₄, filtered, and concentrated to dryness. The residue was purified by flash column chromatography on silica gel to afford tert-butyl ((1S,3R)-3-(2-thioxo-6-(1-((2-(trimethylsilyl)ethoxy)methyl)-1H-1,2,4-triazol-3-yl)-2,3-dihydro-1H-imidazo[4,5-c]pyridin-1-yl)cyclohexyl)carbamate (330 mg, 30% yield). LC-MS: m/z 546 [M+H]⁺.

### Step D. Synthesis of tert-butyl ((1S,3R)-3-(2-(methylthio)-6-(1-((2-(trimethylsilyl)ethoxy)methyl)-1H-1,2,4-triazol-3-yl)-1H-imidazo[4,5-c]pyridin-1-yl)cyclohexyl)carbamate

To a solution of tert-butyl ((1S,3R)-3-(2-thioxo-6-(1-((2-(trimethylsilyl)ethoxy)methyl)-1H-1,2,4-triazol-3-yl)-2,3-dihydro-1H-imidazo[4,5-c]pyridin-1-yl)cyclohexyl)carbamate (310 mg, 568 μmol) in MeCN/THF (10 mL, v/v=1:1) were added DIEA (220 mg, 1.70 mmol, 3 eq.) and Mel (121 mg, 852 μmol, 1.5 eq.). The reaction mixture was stirred at rt for 2 hrs and then concentrated to dryness. The residue was purified by flash column chromatography on silica gel to afford tert-butyl ((1S,3R)-3-(2-(methylthio)-6-(1-((2-(trimethylsilyl)ethoxy)methyl)-1H-1,2,4-triazol-3-yl)-1H-imidazo[4,5-c]pyridin-1-yl)cyclohexyl)carbamate (300 mg, 94% yield). LC-MS: m/z 560 [M+H]⁺.

### Step E. Synthesis of tert-butyl ((1S,3R)-3-(2-(methylsulfonyl)-6-(1-((2-(trimethylsilyl)ethoxy)methyl)-1H-1,2,4-triazol-3-yl)-1H-imidazo[4,5-c]pyridin-1-yl)cyclohexyl)carbamate

To a solution of tert-butyl ((1S,3R)-3-(2-(methylthio)-6-(1-((2-(trimethylsilyl)ethoxy)methyl)-1H-1,2,4-triazol-3-yl)-1H-imidazo[4,5-c]pyridin-1-yl)cyclohexyl)carbamate (290 mg, 518 μmol) in DCM (50 mL) was added m-CPBA (316 mg, 85% wt, 1.55 mmol, 3 eq.), and the reaction mixture was stirred at rt for 16 hrs. The reaction mixture was then quenched by the addition of sat. aq. Na₂SO₃ solution (40 mL), and the aqueous portion extracted with DCM (50 mL×3). The combined organic layers were washed with sat. aq. NaHCO₃ solution (50 mL), dried over Na₂SO₄, filtered, and concentrated to dryness. The residue was purified by flash column chromatography on silica gel to afford tert-butyl ((1S,3R)-3-(2-(methylsulfonyl)-6-(1-((2-(trimethylsilyl)ethoxy)methyl)-1H-1,2,4-triazol-3-yl)-1H-imidazo[4,5-c]pyridin-1-yl)cyclohexyl)carbamate (140 mg, 46% yield). LC-MS: m/z 592 [M+H]⁺.

### Step F. Synthesis of tert-butyl 3-((1-((1R,3S)-3-((tert-butoxycarbonyl)amino)cyclohexyl)-6-(1-((2-(trimethylsilyl)ethoxy)methyl)-1H-1,2,4-triazol-3-yl)-1H-imidazo[4,5-c]pyridin-2-yl)oxy)-1H-pyrazole-1-carboxylate

To a solution of tert-butyl ((1S,3R)-3-(2-(methylsulfonyl)-6-(1-((2-(trimethylsilyl)ethoxy)methyl)-1H-1,2,4-triazol-3-yl)-1H-imidazo[4,5-c]pyridin-1-yl)cyclohexyl)carbamate (100 mg, 169 μmol) and tert-butyl 3-hydroxy-1H-pyrazole-1-carboxylate (46.7 mg, 253 μmol, 1.5 eq.) in DMF (2 mL) was added Cs₂CO₃ (165 mg, 507 μmol, 3 eq.). The reaction mixture was stirred at 80° C. for 2 hrs, then allowed to cool. Water was added (10 mL), and the aqueous portion extracted with EtOAc (20 mL×3). The combined organic layers were washed with H₂O (10 mL), dried over Na₂SO₄, filtered, and concentrated to dryness. The residue was purified by flash column chromatography on silica gel to afford tert-butyl 3-((1-((1R,3S)-3-((tert-butoxycarbonyl)amino)cyclohexyl)-6-(1-((2-(trimethylsilyl)ethoxy)methyl)-1H-1,2,4-triazol-3-yl)-1H-imidazo[4,5-c]pyridin-2-yl)oxy)-1H-pyrazole-1-carboxylate (60 mg, 51% yield). LC-MS: m/z 696 [M+H]⁺.

### Step G. Synthesis of (1S,3R)-3-(2-((1H-pyrazol-3-yl)oxy)-6-(1H-1,2,4-triazol-3-yl)-1H-imidazo[4,5-c]pyridin-1-yl)cyclohexan-1-amine

To a solution of tert-butyl 3-((1-((1R,3S)-3-((tert-butoxycarbonyl)amino)cyclohexyl)-6-(1-((2-(trimethylsilyl)ethoxy)methyl)-1H-1,2,4-triazol-3-yl)-1H-imidazo[4,5-c]pyridin-2-yl)oxy)-1H-pyrazole-1-carboxylate (50 mg, 72 μmol) in DCM (2 mL) was added TFA (2 mL). The reaction mixture was stirred at rt for 2 hrs, and then concentrated to dryness to give (1S,3R)-3-(2-((1H-pyrazol-3-yl)oxy)-6-(1H-1,2,4-triazol-3-yl)-1H-imidazo[4,5-c]pyridin-1-yl)cyclohexan-1-amine (25 mg, TFA salt), which was used without further purification. LC-MS: m/z 366 [M+H]⁺.

### Step H. Synthesis of N-((1S,3R)-3-(2-((1H-pyrazol-3-yl)oxy)-6-(1H-1,2,4-triazol-3-yl)-1H-imidazo[4,5-c]pyridin-1-yl)cyclohexyl)-5-chlorothiazole-2-carboxamide

To a solution of (1S,3R)-3-(2-((1H-pyrazol-3-yl)oxy)-6-(1H-1,2,4-triazol-3-yl)-1H-imidazo[4,5-c]pyridin-1-yl)cyclohexan-1-amine (30 mg, 80 μmol) and lithium 5-chlorothiazole-2-carboxylate (21 mg, 0.12 mmol, 1.5 eq.) in DMF (5 mL) were added pyridine (19 mg, 0.25 mmol, 3 eq.), EDCI (24 mg, 0.12 mmol, 1.5 eq.) and HOBt (19 mg, 0.12 mmol, 1.5 eq.). The reaction mixture was stirred at rt for 24 hrs. Water was added (10 mL), and the aqueous portion extracted with EtOAc (10 mL×3). The combined organic layers were washed with brine (10 mL), dried over Na₂SO₄, filtered, and concentrated to dryness. The residue was purified by prep-HPLC to afford N-((1S,3R)-3-(2-((1H-pyrazol-3-yl)oxy)-6-(1H-1,2,4-triazol-3-yl)-1H-imidazo[4,5-c]pyridin-1-yl)cyclohexyl)-5-chlorothiazole-2-carboxamide (12 mg, 29% yield). ¹H NMR (400 MHz, DMSO-d₆) δ 12.69 (s, 1H), 9.09 (d, 1H), 8.78 (s, 1H), 8.38 (s, 1H), 8.22-7.97 (m, 2H), 7.85-7.67 (m, 1H), 6.42-6.23 (m, 1H), 4.94-4.65 (m, 1H), 4.24-4.01 (m, 1H), 2.23-1.73 (m, 6H), 1.72-1.48 (m, 2H); LC-MS: m/z 511.2 [M+H]⁺.

### Preparation of Example 192 Via General Scheme VIa

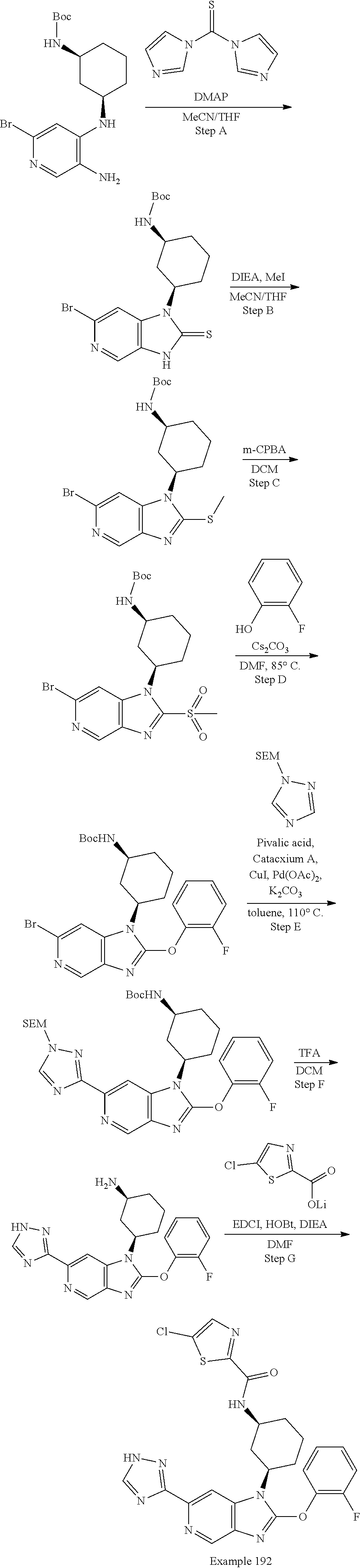

### Step A. Synthesis of tert-butyl ((1S,3R)-3-(6-bromo-2-thioxo-2,3-dihydro-1H-imidazo[4,5-c]pyridin-1-yl)cyclohexyl)carbamate

To a solution of tert-butyl ((1S,3R)-3-((5-amino-2-bromopyridin-4-yl)amino)cyclohexyl)carbamate (2.6 g, 6.75 mmol) in MeCN/THF (35 mL, v/v=1:1) were added DMAP (0.82 g, 6.75 mmol) and di(1H-imidazol-1-yl)methanethione (1.56 g, 8.77 mmol, 1.3 eq.) at 0° C. The reaction mixture was warmed to rt and stirred for 15 hrs. Water (50 mL) was added, and the aqueous portion extracted with EtOAc (50 mL×3). The combined organic layers were washed with brine (50 mL), dried over Na₂SO₄, filtered, and concentrated to dryness. The residue was purified by flash column chromatography on silica gel to afford tert-butyl ((1S,3R)-3-(6-bromo-2-thioxo-2,3-dihydro-1H-imidazo[4,5-c]pyridin-1-yl)cyclohexyl)carbamate (2.3 g, 80% yield). LC-MS: m/z 427 [M+H]⁺.

### Step B. Synthesis of tert-butyl ((1S,3R)-3-(6-bromo-2-(methylthio)-1H-imidazo[4,5-c]pyridin-1-yl)cyclohexyl)carbamate

To a solution of tert-butyl ((1S,3R)-3-(6-bromo-2-thioxo-2,3-dihydro-1H-imidazo[4,5-c]pyridin-1-yl)cyclohexyl)carbamate (2.4 g, 5.61 mmol) in MeCN/THF (30 mL, v/v=1:1) were added DIEA (0.87 g, 6.74 mmol, 1.2 eq.) and Mel (2.39 g, 16.8 mmol, 3 eq.) at 0° C. The reaction mixture was warmed to rt and stirred for 15 hrs. Water (50 mL) was added, and the aqueous portion extracted with EtOAc (30 mL×3). The combined organic layers were washed with brine (50 mL), dried over Na₂SO₄, filtered, and concentrated to dryness. The residue was purified by flash column chromatography on silica gel to afford tert-butyl ((1S,3R)-3-(6-bromo-2-(methylthio)-1H-imidazo[4,5-c]pyridin-1-yl)cyclohexyl)carbamate (2.3 g, 93% yield). LC-MS: m/z 441 [M+H]⁺.

### Step C. Synthesis of tert-butyl ((1S,3R)-3-(6-bromo-2-(methylsulfonyl)-1H-imidazo[4,5-c]pyridin-1-yl)cyclohexyl)carbamate

To a solution of tert-butyl ((1S,3R)-3-(6-bromo-2-(methylthio)-1H-imidazo[4,5-c]pyridin-1-yl)cyclohexyl)carbamate (2.1 g, 4.76 mmol) in DCM (50 mL) was added m-CPBA (2.90 g, 14.3 mmol, 3 eq.) and the reaction mixture was stirred at rt for 5 hrs. The reaction mixture was quenched by the addition of sat. aq. Na₂SO₃ solution (40 mL) and the aqueous portion extracted with DCM (50 mL×3). The combined organic layers were washed with sat. aq. NaHCO₃ solution (50 mL), dried over Na₂SO₄, filtered, and concentrated to dryness. The residue was purified by flash column chromatography on silica gel to afford tert-butyl ((1S,3R)-3-(6-bromo-2-(methylsulfonyl)-1H-imidazo[4,5-c]pyridin-1-yl)cyclohexyl)carbamate (2.2 g, 98% yield). LC-MS: m/z 473 [M+H]⁺.

### Step D. Synthesis of tert-butyl ((1S,3R)-3-(6-bromo-2-(2-fluorophenoxy)-1H-imidazo[4,5-c]pyridin-1-yl)cyclohexyl)carbamate

To a solution of tert-butyl ((1S,3R)-3-(6-bromo-2-(methylsulfonyl)-1H-imidazo[4,5-c]pyridin-1-yl)cyclohexyl)carbamate (1 g, 2.11 mmol) and 2-fluorophenol (0.3 mL, 3.17 mmol, 1.5 eq.) in DMF (12 mL) was added Cs₂CO₃ (2.06 g, 6.34 mmol, 3 eq.) at rt. The reaction mixture was stirred at 85° C. for 12 hrs and then allowed to cool. The reaction mixture was quenched by the addition of sat. aq. NH₄Cl solution (40 mL), and the aqueous portion extracted with EtOAc (50 mL×3). The combined organic layers were washed with H₂O (20 mL), dried over Na₂SO₄, filtered and concentrated to dryness. The residue was purified by flash column chromatography on silica gel to afford tert-butyl ((1S,3R)-3-(6-bromo-2-(2-fluorophenoxy)-1H-imidazo[4,5-c]pyridin-1-yl)cyclohexyl)carbamate (850 mg, 80% yield). LC-MS: m/z 505 [M+H]⁺.

### Step E. Synthesis of tert-butyl ((1S,3R)-3-(2-(2-fluorophenoxy)-6-(1-((2-(trimethylsilyl)ethoxy)methyl)-1H-1,2,4-triazol-3-yl)-1H-imidazo[4,5-c]pyridin-1-yl)cyclohexyl)carbamate

To a solution of tert-butyl ((1S,3R)-3-(6-bromo-2-(2-fluorophenoxy)-1H-imidazo[4,5-c]pyridin-1-yl)cyclohexyl)carbamate (400 mg, 0.79 mmol), 1-((2-(trimethylsilyl)ethoxy)methyl)-1H-1,2,4-triazole (237 mg, 1.19 mmol, 1.5 eq.), pivalic acid (0.045 mL, 0.34 mmol, 0.4 eq.), CuI (30 mg, 0.16 mmol, 0.2 eq.) and K₂CO₃ (328 mg, 2.37 mmol, 3 eq.) in toluene (6 mL) were added Pd(OAc)₂ (36 mg, 0.16 mmol, 0.2 eq.) and Catacxium A (57 mg, 0.16 mmol, 0.2 eq.) at rt. The reaction mixture was then stirred at 110° C. for 24 hrs in tube under N₂. The reaction mixture was concentrated to dryness. The residue was purified by flash column chromatography on silica gel to afford tert-butyl ((1S,3R)-3-(2-(2-fluorophenoxy)-6-(1-((2-(trimethylsilyl)ethoxy)methyl)-1H-1,2,4-triazol-3-yl)-1H-imidazo[4,5-c]pyridin-1-yl)cyclohexyl)carbamate (350 mg, 50% yield). LC-MS: m/z 624 [M+H]⁺.

### Step F. Synthesis of (1S,3R)-3-(2-(2-fluorophenoxy)-6-(1H-1,2,4-triazol-3-yl)-1H-imidazo[4,5-c]pyridin-1-yl)cyclohexan-1-amine

To a solution of tert-butyl ((1S,3R)-3-(2-(2-fluorophenoxy)-6-(1-((2-(trimethylsilyl)ethoxy)methyl)-1H-1,2,4-triazol-3-yl)-1H-imidazo[4,5-c]pyridin-1-yl)cyclohexyl)carbamate (370 mg, 0.42 mmol) in DCM (6 mL) was added TFA (2 mL). The reaction mixture was stirred at rt for 5 hrs and then concentrated to dryness to give (1S,3R)-3-(2-(2-fluorophenoxy)-6-(1H-1,2,4-triazol-3-yl)-1H-imidazo[4,5-c]pyridin-1-yl)cyclohexan-1-amine (350 mg, TFA salt), which was used without further purification. LC-MS: m/z 394 [M+H]⁺.

### Step G. Synthesis of 5-chloro-N-((1S,3R)-3-(2-(2-fluorophenoxy)-6-(1H-1,2,4-triazol-3-yl)-1H-imidazo[4,5-c]pyridin-1-yl)cyclohexyl)thiazole-2-carboxamide

### Example 192

To a solution of (1S,3R)-3-(2-(2-fluorophenoxy)-6-(1H-1,2,4-triazol-3-yl)-1H-imidazo[4,5-c]pyridin-1-yl)cyclohexan-1-amine (350 mg, 0.45 mmol) and lithium 5-chlorothiazole-2-carboxylate (113 mg, 0.67 mmol, 1.5 eq.) in DMF (5 mL) were added DIPEA (345 mg, 2.67 mmol, 6 eq.), EDCI (128 mg, 0.67 mmol, 1.5 eq.) and HOBt (120 mg, 0.89 mmol, 2.0 eq.). The reaction mixture was stirred at rt for 24 hrs. The reaction mixture was quenched by the addition of sat. aq. NH₄Cl solution (20 mL), and the aqueous portion extracted with EtOAc (20 mL×3). The combined organic layers were washed with brine (50 mL), dried over Na₂SO₄, filtered, and concentrated to dryness. The residue was purified by prep-HPLC to afford Example 192 (56.1 mg, 23% yield). ¹H NMR (400 MHz, DMSO-d₆) δ 14.57 (s, 1H), 9.08 (d, 1H), 8.76 (s, 1H), 8.41 (s, 1H), 8.07 (s, 2H), 7.71-7.63 (m, 1H), 7.53-7.31 (m, 3H), 4.96-4.86 (m, 1H), 4.28-4.13 (m, 1H), 2.21-1.98 (m, 3H), 1.97-1.50 (m, 5H); LC-MS: m/z 539.1 [M+H]⁺.

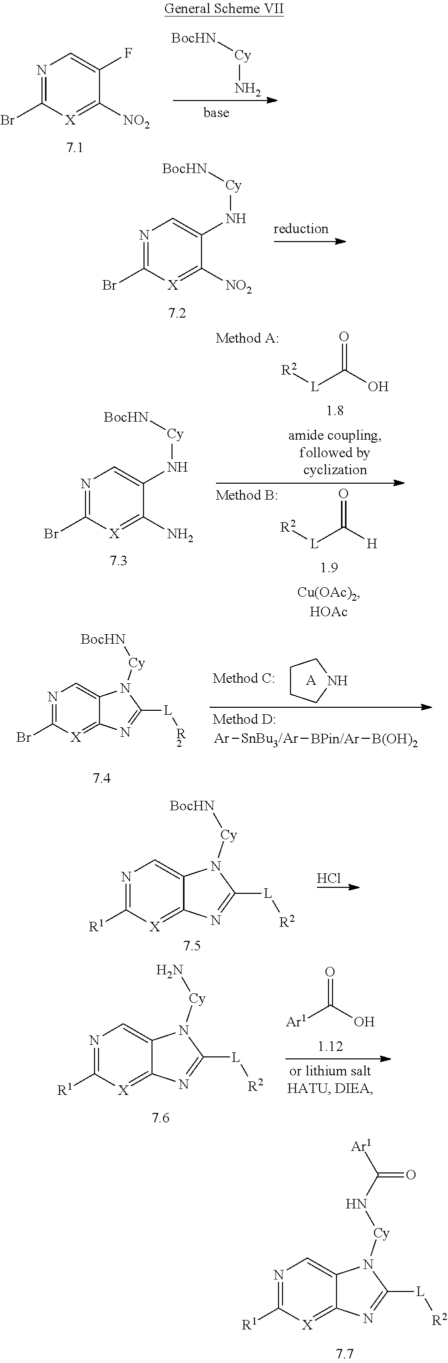

Compounds of formula 7.7 were obtained through the scheme depicted as General Scheme VII. Beginning with the 2,5-di-halo-4-nitropyridine of formula 7.1, a nucleophilic aromatic substitution reaction conducted under basic conditions was used to couple N-Boc-1,3-Cy-diamine compound of formula 1.2 to afford compounds of formula 7.1. The nitro group in compound of formula 7.1 was reduced to afford the pyridine-diamine compound of formula 7.3. Subsequently, various L and R² groups were installed by coupling the pyridine-diamine compound of formula 7.3 with acid 1.8 to form an amide intermediate (Method A), which was subsequently cyclized under either basic or acidic conditions to form compounds of formula 7.4. Alternatively, the pyridine-diamine compound of formula 7.3 was condensed with aldehyde 1.9 (Method B), followed by cyclization and in situ oxidation to afford compound 7.4. R¹ groups wherein the R¹ group is a five-membered heteroaryl or heterocycloalkyl containing at least one nitrogen were installed by displacing the bromide in compounds of formula 7.4 with the NH of the heteroaryl or heterocycloalkyl reagent 1.5 in the presence of a suitable base and/or metal catalysts (Method C) to form compounds of formula 7.5. Alternatively, using Method D, the bromide in compound 7.4 was converted to an heteroaryl group via a Suzuki coupling of heteroaryl boronic pinacol esters or boronic acids to afford compounds of formula 7.5. The Boc protecting group of compounds of formula 7.5 was then removed under acidic conditions to form compounds of formula 7.6. Ar¹ groups were then installed by reacting an Ar¹ carboxylic acid of formula 1.12 or a basic salt (i.e., Li, K, or Na) thereof with an amine of formula 7.6 using acid coupling conditions known in the art, such as using one of the following reagents—HATU, EDCI, HOBt—along with a base, such as DIPEA (Hunig's base), pyridine, or TEA to form compounds of formula 7.7.

### Preparation of Examples 148 and 149 Via General Scheme VII (Methods B and C)

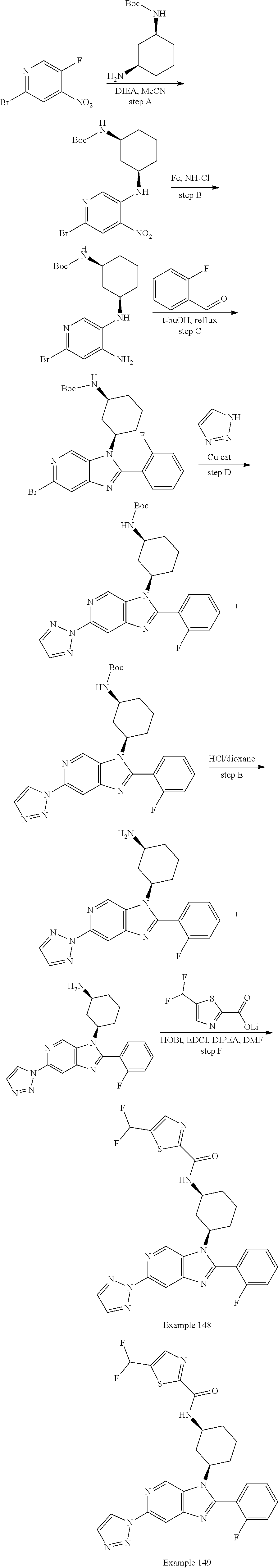

### Step A. Synthesis of tert-butyl ((1S,3R)-3-((6-bromo-4-nitropyridin-3-yl)amino)cyclohexyl)carbamate

A suspension of 2-bromo-5-fluoro-4-nitropyridine (3.32 g, 14.0 mmol), tert-butyl ((1S,3R)-3-aminocyclohexyl)carbamate (3.00 g, 14.0 mmol) and DIEA (3.62 g, 28.0 mmol) in MeCN (30 mL) was stirred at 30° C. for 16 hrs. The reaction mixture was concentrated to dryness to give tert-butyl ((1S,3R)-3-((6-bromo-4-nitropyridin-3-yl)amino)cyclohexyl)carbamate, which was used without further purification (6 g). LC-MS. m/z 375.0 [M−55]⁺.

### Step B. Synthesis of tert-butyl ((1S,3R)-3-((4-amino-6-bromopyridin-3-yl)amino)cyclohexyl)carbamate

To a suspension of tert-butyl ((1S,3R)-3-((6-bromo-4-nitropyridin-3-yl)amino)cyclohexyl)carbamate (6.00 g, 13.9 mmol) and Fe (3.88 g, 69.6 mmol) in THF (10 mL) was added NH₄Cl (7.44 g, 139 mmol) in H₂O (5 mL). After the addition was completed, the reaction mixture was stirred at 80° C. for 16 hrs, and then allowed to cool. The resulting slurry was filtered, and water (150 mL) was added into the filtrate. The resulting aqueous portion was extracted with EtOAc (3×120 mL). The combined organic layers were washed with brine (150 mL), dried over Na₂SO₄, filtered, and concentrated to dryness. The residue was purified by column chromatography on silica gel to give tert-butyl ((1S,3R)-3-((4-amino-6-bromopyridin-3-yl)amino)cyclohexyl)carbamate (4.6 g). LC-MS: m/z 387.1 [M+H]⁺.

### Step C. Synthesis of tert-butyl ((1S,3R)-3-(6-bromo-2-(2-fluorophenyl)-3H-imidazo[4,5-c]pyridin-3-yl)cyclohexyl)carbamate

A solution of tert-butyl ((1S,3R)-3-((4-amino-6-bromopyridin-3-yl)amino)cyclohexyl)carbamate (300 mg, 778 μmol) and 2-fluorobenzaldehyde (97.4 mg, 779 μmol) in t-BuOH (5 mL) was heated at 100° C. for 16 hrs. The reaction mixture was concentrated to dryness to give a residue, which was purified by column chromatography on silica gel to give tert-butyl N-[(1S,3R)-3-[6-bromo-2-(2-fluorophenyl)imidazo[4,5-c]pyridin-3-yl]cyclohexyl]carbamate (253 mg, 61.5% yield). LC-MS: m/z 492.2 [M+H]⁺.

### Step D. Synthesis of tert-butyl ((1S,3R)-3-(2-(2-fluorophenyl)-6-(2H-1,2,3-triazol-2-yl)-3H-imidazo[4,5-c]pyridin-3-yl)cyclohexyl)carbamate and tert-butyl ((1S,3R)-3-(2-(2-fluorophenyl)-6-(1H-1,2,3-triazol-1-yl)-3H-imidazo[4,5-c]pyridin-3-yl)cyclohexyl) carbamate

A mixture of tert-butyl N-[(1S,3R)-3-[6-bromo-2-(2-fluorophenyl)imidazo[4,5-c]pyridin-3-yl]cyclohexyl]carbamate (460 mg, 940 μmol), 1H-triazole (260 mg, 3.76 mmol), N,N′-bis(2-furylmethyl)oxamide (46.7 mg, 188 μmol), Cu₂O (53.8 mg, 376 μmol) and K₃PO₄ (399 mg, 1.88 mmol) in DMSO (5 mL) was degassed and purged with N₂ for 3 times, and then the reaction mixture was stirred at 120° C. for 14 hrs under N₂ atmosphere. The reaction mixture was allowed to cool and then quenched by the addition of brine (30 mL), and the aqueous portion extracted with EtOAc (30 mL×3). The combined organic layers were concentrated under reduced pressure, and purified by column chromatography to give a mixture of tert-butyl ((1S,3R)-3-(2-(2-fluorophenyl)-6-(2H-1,2,3-triazol-2-yl)-3H-imidazo[4,5-c]pyridin-3-yl)cyclohexyl)carbamate and tert-butyl ((1S,3R)-3-(2-(2-fluorophenyl)-6-(1H-1,2,3-triazol-1-yl)-3H-imidazo[4,5-c]pyridin-3-yl)cyclohexyl) carbamate (110 mg), which was used without further purification. LC-MS: m/z 478.2 [M+H]⁺.

### Step E. Synthesis of (1S,3R)-3-(2-(2-fluorophenyl)-6-(2H-1,2,3-triazol-2-yl)-3H-imidazo[4,5-c]pyridin-3-yl)cyclohexan-1-amine and (1S,3R)-3-(2-(2-fluorophenyl)-6-(1H-1,2,3-triazol-1-yl)-3H-imidazo[4,5-c]pyridin-3-yl)cyclohexan-1-amine

A mixture of tert-butyl ((1S,3R)-3-(2-(2-fluorophenyl)-6-(2H-1,2,3-triazol-2-yl)-3H-imidazo[4,5-c]pyridin-3-yl)cyclohexyl)carbamate and tert-butyl ((1S,3R)-3-(2-(2-fluorophenyl)-6-(1H-1,2,3-triazol-1-yl)-3H-imidazo[4,5-c]pyridin-3-yl)cyclohexyl) carbamate (110 mg, 230 μmol) in HCl/dioxane (1.5 mL) was stirred at rt for 1 hr. The reaction mixture was concentrated under reduced pressure to give a mixture of (1S,3R)-3-(2-(2-fluorophenyl)-6-(2H-1,2,3-triazol-2-yl)-3H-imidazo[4,5-c]pyridin-3-yl)cyclohexan-1-amine and (1S,3R)-3-(2-(2-fluorophenyl)-6-(1H-1,2,3-triazol-1-yl)-3H-imidazo[4,5-c]pyridin-3-yl)cyclohexan-1-amine (130 mg), which was used without further purification. LC-MS: m/z 378.2 [M+H]⁺.

### Step F. Synthesis of 5-(difluoromethyl)-N-((1S,3R)-3-(2-(2-fluorophenyl)-6-(2H-1,2,3-triazol-2-yl)-3H-imidazo[4,5-c]pyridin-3-yl)cyclohexyl)thiazole-2-carboxamide (Example 148) and 5-(difluoromethyl)-N-((1S,3R)-3-(2-(2-fluorophenyl)-6-(1H-1,2,3-triazol-1-yl)-3H-imidazo[4,5-c]pyridin-3-yl)cyclohexyl)thiazole-2-carboxamide Example 149

A mixture of (1S,3R)-3-(2-(2-fluorophenyl)-6-(2H-1,2,3-triazol-2-yl)-3H-imidazo[4,5-c]pyridin-3-yl)cyclohexan-1-amine and (1S,3R)-3-(2-(2-fluorophenyl)-6-(1H-1,2,3-triazol-1-yl)-3H-imidazo[4,5-c]pyridin-3-yl)cyclohexan-1-amine (130 mg, 344 μmol), [5-(difluoromethyl)thiazole-2-carbonyl]oxylithium (63.8 mg, 344 μmol), DIEA (178 mg, 1.38 mmol), HOBt (69.8 mg, 517 μmol) and EDCI (99.1 mg, 517 μmol) in DMF (1 mL) was stirred at rt for 14 hrs. The reaction mixture was quenched by the addition of brine (5 mL), and the aqueous portion was extracted with EtOAc (10 mL×3). The combined organic layers were concentrated to dryness, and the residue purified by column chromatography, followed by prep-HPLC (column: Welch Xtimate C18 150*25 mm*5 um; mobile phase: [water (0.225% FA)-ACN]; B %: 21%-51%, 6 min), and then by SFC (column: DAICEL CHIRALPAK AD (250 mm*30 mm, 10 um); mobile phase: [0.1% NH₃H₂O IPA]; B %: 50%-50%, min) to give 5-(difluoromethyl)-N-((1S,3R)-3-(2-(2-fluorophenyl)-6-(2H-1,2,3-triazol-2-yl)-3H-imidazo[4,5-c]pyridin-3-yl)cyclohexyl)thiazole-2-carboxamide (Example 148) (8.6 mg, 42.2% yield) and 5-(difluoromethyl)-N-((1S,3R)-3-(2-(2-fluorophenyl)-6-(1H-1,2,3-triazol-1-yl)-3H-imidazo[4,5-c]pyridin-3-yl)cyclohexyl)thiazole-2-carboxamide (Example 149) (10 mg, 49.4% yield).
Example 148: ¹H NMR (400 MHz, CD₃OD) δ 9.18 (s, 1H), 8.36 (s, 1H), 8.15 (s, 1H), 8.04 (s, 2H), 7.81-7.70 (m, 2H), 7.53-7.41 (m, 2H), 7.32-7.01 (m, 1H), 4.40-4.33 (m, 1H), 4.08-3.97 (m, 1H), 2.58-2.48 (m, 1H), 2.40-2.26 (m, 2H), 2.14-1.99 (m, 3H), 1.74-1.44 (m, 2H), LC-MS: m/z 539.2 [M+H]⁺.
Example 149: ¹H NMR (400 MHz, CD₃OD) δ 9.25 (s, 1H), 8.82 (s, 1H), 8.41 (s, 1H), 8.15 (s, 1H), 7.93 (s, 1H), 7.80-7.69 (m, 2H), 7.54-7.42 (d, 2H), 7.32-7.01 (m, 1H), 4.40-4.33 (m, 1H), 4.08-3.95 (m, 1H), 2.59-2.49 (m, 1H), 2.40-2.26 (t, 2H), 2.13-1.98 (m, 3H), 1.73-1.44 (m, 2H). LC-MS: m/z 539.2 [M+H]⁺.

General Scheme VII set forth above was used to synthesize the following compounds by using appropriate starting materials:

| Ex. | | | LC-MS: |
|---|---|---|---|
| No. | Structure | ¹H NMR | m/z |
| 150 | 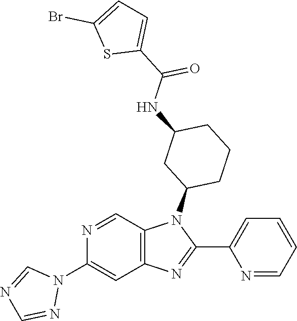 [SMILES: O=C(N[C@H]1CCC[C@@H](n2c(-c3ccccn3)nc3cc(-n4cncn4)ncc32)C1)c1ccc(Br)s1] | (400 MHz, CD₃OD) δ 9.34 (s, 1 H), 9.17 (s, 1 H), 8.85 (d, 1 H), 8.21-8.19 (m, 3 H), 8.05 (t, 1 H), 7.59 (t, 1 H), 7.48 (d, 1 H), 7.12 (d, 1 H), 5.61 (br s, 1 H), 4.04 (br s, 1 H), 2.48-2.34 (m, 2 H), 2.31-2.26 (m, 1 H), 2.21-2.17 (m, 1 H), 2.10-1.99 (m, 2 H), 1.65-1.55 (m, 2 H). | 551.2 [M + H]⁺ |
| 151 | 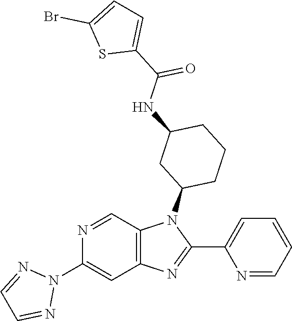 [SMILES: O=C(N[C@H]1CCC[C@@H](n2c(-c3ccccn3)nc3cc(-n4nccn4)ncc32)C1)c1ccc(Br)s1] | (400 MHz, CD₃OD) δ 9.15 (s, 1 H), 8.84 (s, 1 H), 8.35 (s, 1 H), 8.21 (s, 1 H), 8.05 (t, 1 H), 8.03 (s, 2 H), 7.61 (s, 1 H), 7.49 (d, 1 H), 7.12 (d, 1 H), 5.61 (br s, 1 H), 4.04 (br s, 1 H), 2.48-2.39 (m, 2 H), 2.31- 2.26 (m, 1 H), 2.21-2.17 (m, 1 H), 2.10- 1.99 (m, 2 H), 1.65-1.55 (m, 2 H). | 551.2 [M + H]⁺ |
| 152 | 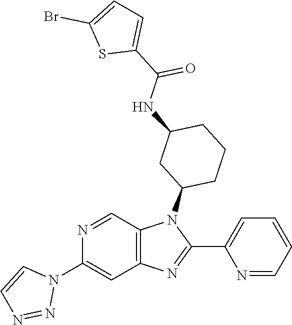 [SMILES: O=C(N[C@H]1CCC[C@@H](n2c(-c3ccccn3)nc3cc(-n4ccnn4)ncc32)C1)c1ccc(Br)s1] | (400 MHz, CD₃OD) δ 9.21 (s, 1 H), 8.84 (d, 1 H), 8.80 (s, 1 H), 8.40 (s, 1 H), 8.24 (d, 1 H), 8.08 (t, 1 H), 7.92 (s, 1 H), 7.63- 7.60 (m, 1 H), 7.49 (d, 1 H), 7.12 (d, 1 H), 5.61 (br s, 1 H), 4.04 (br s, 1 H), 2.50-2.41 (m, 2 H), 2.31-2.26 (m, 1 H), 2.21-2.17 (m, 1 H), 2.07-2.05 (m, 2 H), 1.65-1.55 (m, 2 H). | 551.2 [M + H]⁺ |
| 153 | 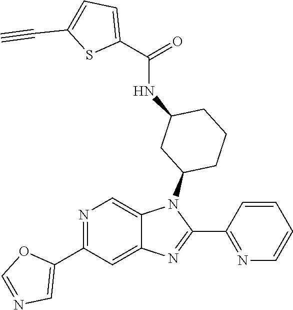 [SMILES: C#Cc1ccc(C(=O)N[C@H]2CCC[C@@H](n3c(-c4ccccn4)nc4cc(-c5cnco5)ncc43)C2)s1] | (400 MHz, CDCl₃) δ 9.16 (d, 1 H), 8.80 (dd, 1 H), 8.34 (d, 1 H), 8.11 (s, 1 H), 8.03-7.91(m, 2 H), 7.68 (s, 1 H), 7.49 (ddd, 1 H), 7.34 (d, 1 H), 7.21 (d, 1 H), 5.99-5.83 (m, 2 H), 4.20 (br d, 1 H), 3.45 (s, 1 H), 2.58 (br d, 1 H), 2.34-2.21 (m, 4 H), 2.16-2.06 (m, 1 H). | 495.4 [M + H]⁺ |
| 154 | 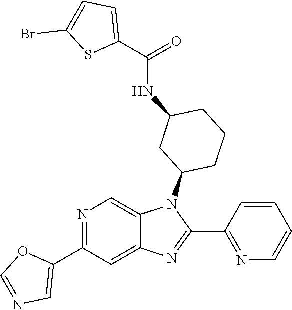 [SMILES: O=C(N[C@H]1CCC[C@@H](n2c(-c3ccccn3)nc3cc(-c4cnco4)ncc32)C1)c1ccc(Br)s1] | (400 MHz, CDCl₃) δ 9.15 (s, 1 H), 8.79 (dd, 1 H), 8.33 (d, 1 H), 8.11 (s, 1 H), 8.03-7.93 (m, 2 H), 7.68 (s, 1 H), 7.49 (ddd, 1 H), 7.23 (d, 1 H), 7.04 (d, 1 H), 6.00-5.82 (m, 2 H), 4.19 (br d, 1 H), 2.57 (br d, 1 H), 2.32-2.19 (m, 4 H), 2.15-2.06 (m, 1 H), 1.62 (br s, 1 H), 1.40 (br dd, 1 H) | 551.2 [M + H]⁺ |
| 156 | 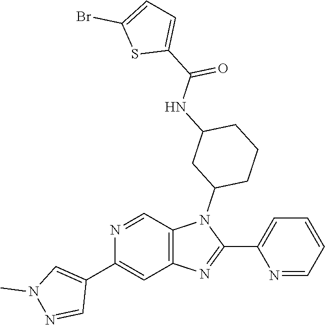 [SMILES: Cn1cc(-c2cc3nc(-c4ccccn4)n(C4CCCC(NC(=O)c5ccc(Br)s5)C4)c3cn2)cn1] | (400 MHz, CD₃OD) δ 9.15 (s, 1 H), 8.86 (d, 1 H), 8.21-8.19 (m, 2 H), 8.11-8.03 (m, 2 H), 7.95 (s, 1 H), 7.65-7.63 (m, 1 H), 7.52 (d, 1 H), 7.15 (d, 1 H), 5.55 (br s, 1 H), 4.07 (br s, 1 H), 3.99 (s, 3 H), 2.49- 2.41 (m, 2 H), 2.39-2.11 (m, 2 H), 2.05 (d, 2 H), 1.65-1.55 (m, 2 H). | 564.2 [M + H]⁺ |
| | (relative stereochemistry at | | |
| | centers 1 and 3 of cyclohexyl | | |
| | ring is cis) | | |

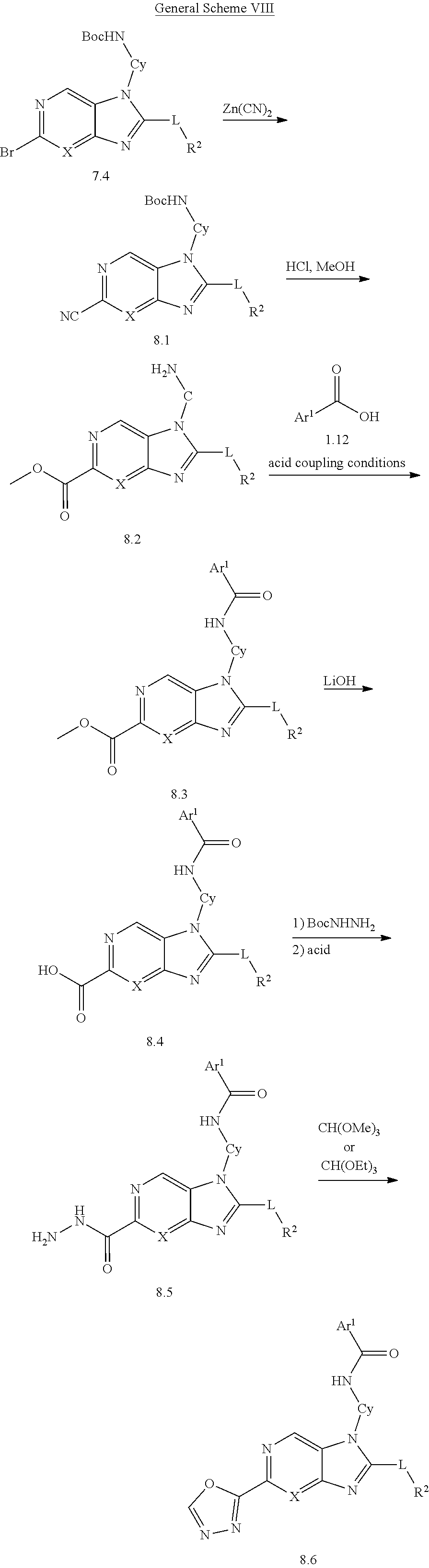

Compounds of formula 8.6 were obtained through the scheme depicted as General Scheme VIII. Beginning with compounds of formula 7.4 (prepared in accordance with General Scheme VII), the bromide in compounds of formula 7.4 was converted to a cyano group using zinc cyanide to afford compounds of formula 8.1. The cyano group in compounds of formula 8.1 was then converted to a methyl ester using a strong acid such as HCl in the presence of MeOH, and the amino Boc protecting group removed concomitantly under acidic conditions to afford compounds of formula 8.2. Ar¹ groups were then installed by reacting an Ar¹ carboxylic acid of formula 1.12 or a basic salt (i.e., Li, K, or Na) thereof with an amine of formula 8.2 using acid coupling conditions known in the art, such as using one of the following reagents—HATU, EDCI, HOBt—along with a base, such as DIPEA (Hunig's base), pyridine, or TEA to form compounds of formula 8.3. The methyl ester moiety in compounds of formula 8.4 was hydrolyzed to the acid using lithium hydroxide to afford compounds of formula 8.4. The acid group in compounds of formula 8.4 was then converted to a hydrazide group to afford compounds of formula 8.5. The hydrazide group in compounds of formula 8.5 was then converted to a 1,3,4-oxadiazole heteroaryl with trimethyl orthoformate or triethyl orthoformate to afford compounds of formula 8.6.

### Preparation of Example 157 Via General Scheme VIII (Relative Stereochemistry at Centers 1 and 3 of Cyclohexyl Ring in the Scheme Below is Cis)

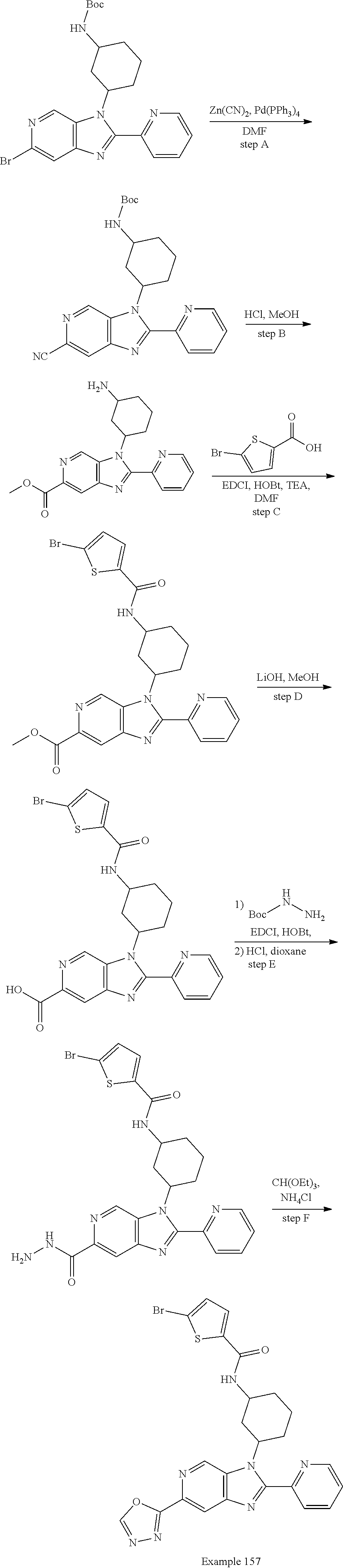

### Step A. tert-butyl (3-(6-cyano-2-(pyridin-2-yl)-3H-imidazo[4,5-c]pyridin-3-yl)cyclohexyl)carbamate

To a mixture of tert-butyl (3-(6-bromo-2-(pyridin-2-yl)-3H-imidazo[4,5-c]pyridin-3-yl)cyclohexyl)carbamate and Zn(CN)₂ (54.7 mg, 466 μmol) in DMF (1 mL) was added Pd(PPh₃)₄ (26.9 mg, 23.3 μmol) under nitrogen. After the addition was complete, the reaction mixture was heated at 130° C. under nitrogen for 16 hrs, then allowed to cool. DCM (40 mL) was added into the reaction mixture, and the organic portion was washed with water (15 mL), brine (15 mL), filtered and concentrated to dryness. The residue was purified by column chromatography on silica gel to give tert-butyl (3-(6-cyano-2-(pyridin-2-yl)-3H-imidazo[4,5-c]pyridin-3-yl)cyclohexyl)carbamate (63 mg). LC-MS: m/z 419.1 [M+H]⁺.

### Step B. Synthesis of methyl 3-(3-aminocyclohexyl)-2-(pyridin-2-yl)-3H-imidazo[4,5-c]pyridine-6-carboxylate

A solution of tert-butyl (3-(6-cyano-2-(pyridin-2-yl)-3H-imidazo[4,5-c]pyridin-3-yl)cyclohexyl)carbamate (63 mg) in HCl/MeOH (4 M, 3.5 mL) was stirred at 70° C. for 1.5 hrs. The reaction mixture was concentrated to dryness to give methyl 3-(3-aminocyclohexyl)-2-(pyridin-2-yl)-3H-imidazo[4,5-c]pyridine-6-carboxylate (60 mg, HCl salt), which was used without any further purification. LC-MS: m/z 352.3 [M+H]⁺.

### Step C. Synthesis of methyl 3-(3-(5-bromothiophene-2-carboxamido)cyclohexyl)-2-(pyridin-2-yl)-3H-imidazo[4,5-c]pyridine-6-carboxylate

To a solution of 5-bromothiophene-2-carboxylic acid (32.0 mg, 155 μmol), methyl 3-(3-aminocyclohexyl)-2-(pyridin-2-yl)-3H-imidazo[4,5-c]pyridine-6-carboxylate (60 mg, HCl salt), HOBt (31.4 mg, 232 μmol) and TEA (31.3 mg, 309 μmol) in DMF (2 mL) was added EDCI (44.5 mg, 232 μmol). After the addition was complete, the reaction mixture was stirred at rt for 16 hrs. The reaction mixture was filtered, and the filtrate was concentrated to dryness. The residue was purified by column chromatography on silica gel to give methyl 3-(3-(5-bromothiophene-2-carboxamido)cyclohexyl)-2-(pyridin-2-yl)-3H-imidazo[4,5-c]pyridine-6-carboxylate (80 mg, 83.9% yield). LC-MS: m/z 540.1 [M+H]⁺.

### Step D. Synthesis of 3-(3-(5-bromothiophene-2-carboxamido)cyclohexyl)-2-(pyridin-2-yl)-3H-imidazo[4,5-c]pyridine-6-carboxylic acid

To a solution of methyl 3-(3-(5-bromothiophene-2-carboxamido)cyclohexyl)-2-(pyridin-2-yl)-3H-imidazo[4,5-c]pyridine-6-carboxylate (80 mg, 130 μmol) in MeOH (4 mL) was added LiOH·H₂O (31.1 mg, 740 μmol) in H₂O (2 mL). After the addition was complete, the reaction mixture was stirred at rt for 16 hrs. The reaction mixture was concentrated to dryness to give 3-(3-(5-bromothiophene-2-carboxamido)cyclohexyl)-2-(pyridin-2-yl)-3H-imidazo[4,5-c]pyridine-6-carboxylic acid (0.08 g, Li salt) which was used without further purification. LC-MS: m/z 528.1 [M+H]⁺.

### Step E. 5-bromo-N-(3-(6-(hydrazinecarbonyl)-2-(pyridin-2-yl)-3H-imidazo[4,5-c]pyridin-3-yl)cyclohexyl)thiophene-2-carboxamide

To a solution of 3-(3-(5-bromothiophene-2-carboxamido)cyclohexyl)-2-(pyridin-2-yl)-3H-imidazo[4,5-c]pyridine-6-carboxylic acid (40 mg, 75 μmol, Li salt), tert-butyl N-aminocarbamate (10.9 mg, 82.5 μmol) and HATU (42.8 mg, 113 μmol) in DMF (1 mL) was added DIEA (9.69 mg, 75.0 μmol). After the addition was complete, the reaction mixture was stirred at rt for 16 hrs. DCM (50 mL) was added, and the organic portion was washed with water (15 mL), brine (15 mL), dried over Na₂SO₄, filtered and concentrated to dryness. The residue was purified by column chromatography on silica gel to give tert-butyl 2-(3-(3-(5-bromothiophene-2-carboxamido)cyclohexyl)-2-(pyridin-2-yl)-3H-imidazo[4,5-c]pyridine-6-carbonyl)hydrazinecarboxylate (50 mg), which was used without further purification. LC-MS: m/z 640.0 [M+H]⁺.

A solution of tert-butyl 2-(3-(3-(5-bromothiophene-2-carboxamido)cyclohexyl)-2-(pyridin-2-yl)-3H-imidazo[4,5-c]pyridine-6-carbonyl) hydrazine carboxylate (50 mg) in HCl/dioxane (4 M, 1 mL) was stirred at rt for 2 hrs. The reaction mixture was concentrated to dryness to give 5-bromo-N-(3-(6-(hydrazinecarbonyl)-2-(pyridin-2-yl)-3H-imidazo[4,5-c]pyridin-3-yl)cyclohexyl)thiophene-2-carboxamide (40 mg, HCl salt) which was used without any further purification. LC-MS: m/z 542.0 [M+H]⁺.

### Step F. Synthesis of N-(3-(6-(1,3,4-oxadiazol-2-yl)-2-(pyridin-2-yl)-3H-imidazo[4,5-c]pyridin-3-yl)cyclohexyl)-5-bromothiophene-2-carboxamide (Example 157)

To a solution of 5-bromo-N-(3-(6-(hydrazinecarbonyl)-2-(pyridin-2-yl)-3H-imidazo[4,5-c]pyridin-3-yl)cyclohexyl)thiophene-2-carboxamide (40 mg, HCl salt) and triethyl orthoformate (891 mg, 6.01 mmol) was added NH₄Cl (3.96 mg, 74.0 μmol). After the addition was complete, the reaction mixture was heated at 80° C. for 16 hrs. The reaction mixture was purified by prep-HPLC (column: Phenomenex Gemini-NX C18 75*30 mm*3 um; mobile phase: [water(0.05% NH₃H₂O+10 mM NH₄HCO₃)-ACN]; B %: 35%-65%, 6 min) to give Example 157 (relative stereochemistry at centers 1 and 3 of cyclohexyl ring is cis) (2.2 mg). ¹H NMR (400 MHz, CD₃OD) δ 9.41 (s, 1H), 9.16 (s, 1H), 8.88 (d, 1H), 8.58 (s, 1H), 8.28 (d, 1H), 8.11 (t, 1H), 7.71-7.62 (m, 1H), 7.52 (d, 1H), 7.16 (d, 1H), 5.71 (br s, 1H), 4.07 (br s, 1H), 2.47 (t, 2H), 2.42-2.21 (m, 2H), 2.10 (d, 2H), 1.71-1.55 (m, 2H). LC-MS: m/z 550.2 [M+H]⁺.

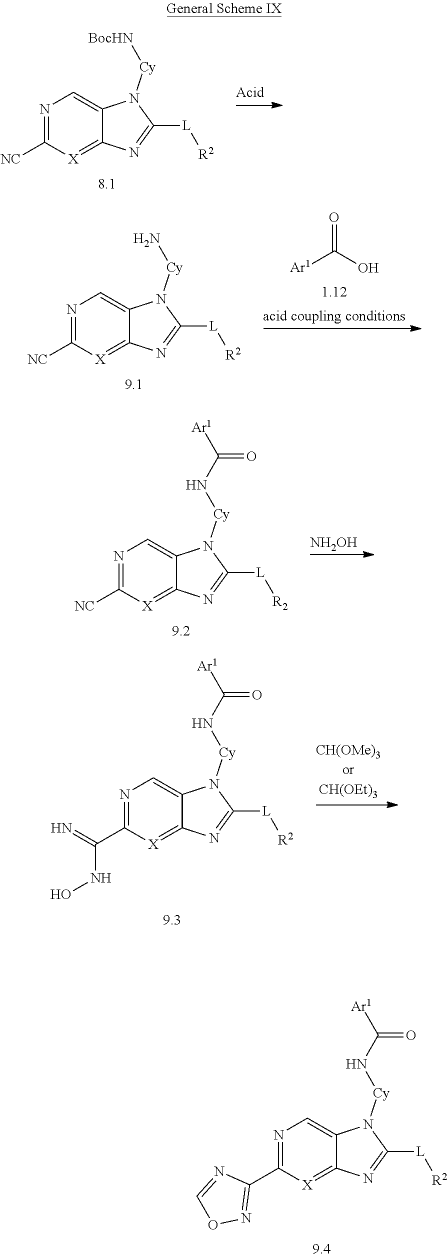

Compounds of formula 9.4 were obtained through the scheme depicted as General Scheme IX. Beginning with compounds of formula 8.1 (prepared in accordance with General Scheme VIII), the amino Boc protecting group in compounds of formula 8.1 was removed under acidic conditions to afford compound 9.1. Ar¹ groups were then installed by reacting an Ar¹ carboxylic acid of formula 1.12 or a basic salt (i.e., Li, K, or Na) thereof with an amine of formula 9.1 using acid coupling conditions known in the art, such as using one of the following reagents—HATU, EDCI, HOBt—along with a base, such as DIPEA (Hunig's base), pyridine, or TEA to form compounds of formula 9.2. The cyano group in compounds of formula 9.2 was reacted with hydroxyamine to form the N-hydroxyguanide moiety of compounds of formula 9.3. The N-hydroxyguanide moiety in compounds of formula 9.3 was then converted to 1,2,4-oxadiazole heteroaryl through reaction with trimethyl orthoformate or triethyl orthoformate to afford compounds of formula 9.4.

### Preparation of Example 155 Via General Scheme IX (Relative Stereochemistry at Centers 1 and 3 of Cyclohexyl Ring in the Scheme Below is Cis)

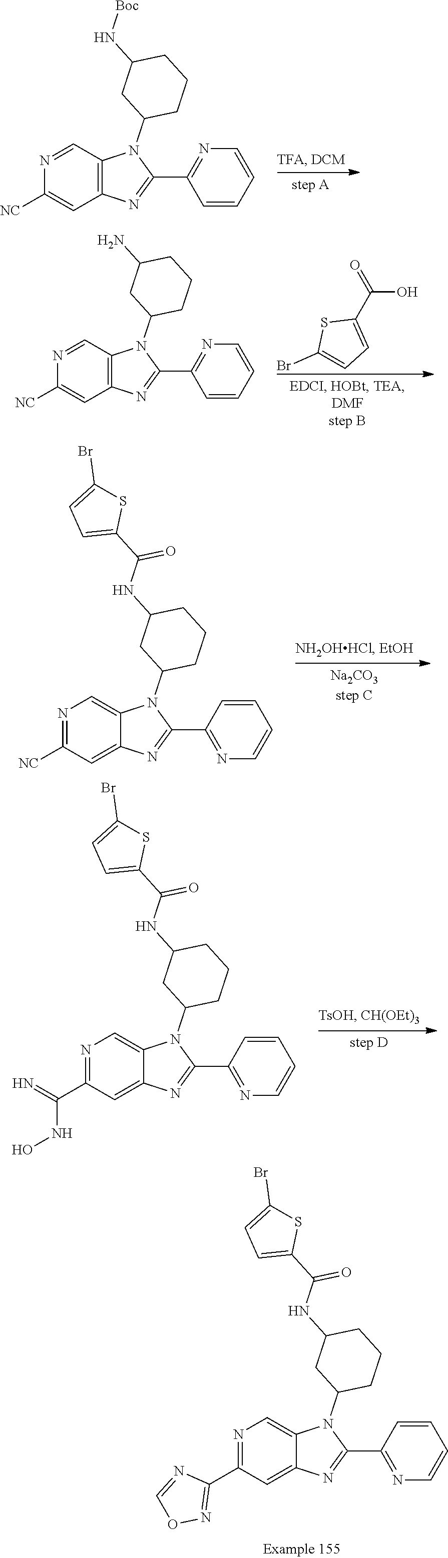

### Step A. Synthesis of 3-(3-aminocyclohexyl)-2-(pyridin-2-yl)-3H-imidazo[4,5-c]pyridine-6-carbonitrile

To a solution of tert-butyl (3-(6-cyano-2-(pyridin-2-yl)-3H-imidazo[4,5-c]pyridin-3-yl)cyclohexyl)carbamate (70 mg, 167 μmol) in DCM (1 mL) was added TFA (95.4 mg, 836 μmol). After the addition was complete, the reaction mixture was stirred at rt for 2 hrs. The reaction mixture was adjusted to pH 9 by the addition of aq. sat. NaHCO₃ solution. The aqueous portion was extracted with DCM:MeOH=10:1 (5×15 mL). The combined organic layers were washed with brine (15 mL), dried over Na₂SO₄, filtered and concentrated to dryness to give 3-(3-aminocyclohexyl)-2-(pyridin-2-yl)-3H-imidazo[4,5-c]pyridine-6-carbonitrile (35 mg), which was used without further purification. LC-MS: m/z 319.2 [M+H]⁺.

### Step B. Synthesis of 5-bromo-N-(3-(6-cyano-2-(pyridin-2-yl)-3H-imidazo[4,5-c]pyridin-3-yl)cyclohexyl)thiophene-2-carboxamide

To a solution of 5-bromothiophene-2-carboxylic acid (22.8 mg, 110 μmol), 3-(3-aminocyclohexyl)-2-(pyridin-2-yl)-3H-imidazo[4,5-c]pyridine-6-carbonitrile (35 mg), HOBt (22.3 mg, 165 μmol) and TEA (22.3 mg, 220 μmol) in DMF (2 mL) was added EDCI (31.6 mg, 165 μmol). After the addition was complete, the reaction mixture was stirred at rt for 16 hrs and then concentrated to dryness. The residue was purified by column chromatography on silica gel to give 5-bromo-N-(3-(6-cyano-2-(pyridin-2-yl)-3H-imidazo[4,5-c]pyridin-3-yl)cyclohexyl)thiophene-2-carboxamide (77 mg), which was used without further purification. LC-MS: m/z 509.1 [M+H]⁺.

### Step C. Synthesis of 5-bromo-N-((1S,3R)-3-(6-(N-hydroxycarbamimidoyl)-2-(pyridin-2-yl)-3H-imidazo[4,5-c]pyridin-3-yl)cyclohexyl)thiophene-2-carboxamide

To a solution of 5-bromo-N-(-3-(6-cyano-2-(pyridin-2-yl)-3H-imidazo[4,5-c]pyridin-3-yl)cyclohexyl)thiophene-2-carboxamide (77 mg, 151.75 μmol) and hydroxylamine (21.1 mg, 304 μmol, HCl salt) in EtOH (2 mL) was added Na₂CO₃ (64.3 mg, 607 μmol). After the addition was complete, the reaction mixture was heated at 80° C. for 16 hrs, and then allowed to cool. The reaction mixture was filtered, and the filtrate was concentrated to dryness to give 5-bromo-N-((1S,3R)-3-(6-(N-hydroxycarbamimidoyl)-2-(pyridin-2-yl)-3H-imidazo[4,5-c]pyridin-3-yl)cyclohexyl)thiophene-2-carboxamide (80 mg), which was used without further purification. LC-MS: m/z 542.1 [M+H]⁺.

### Step D. Synthesis of N-(3-(6-(1,2,4-oxadiazol-3-yl)-2-(pyridin-2-yl)-3H-imidazo[4,5-c]pyridin-3-yl)cyclohexyl)-5-bromothiophene-2-carboxamide (Example 155)

To a mixture of 5-bromo-N-(3-(6-(N-hydroxycarbamimidoyl)-2-(pyridin-2-yl)-3H-imidazo[4,5-c]pyridin-3-yl)cyclohexyl)thiophene-2-carboxamide (80 mg) in triethoxymethane (1 mL) was added TsOH·H₂O (14.1 mg, 74.0 μmol). After the addition was complete, the reaction mixture was heated at 100° C. for 16 hrs, and then allowed to cool. The reaction mixture was filtered, and the filtrate was concentrated to dryness. The residue was purified by prep-TLC to give Example 155 (relative stereochemistry at centers 1 and 3 of cyclohexyl ring is cis) (13.4 mg, 14.9% yield). ¹H NMR (400 MHz, CD₃OD) δ 9.30 (s, 1H), 9.25 (s, 1H), 8.76 (d, 1H), 8.45 (s, 1H), 8.14 (d, 1H), 8.01-7.98 (m, 1H), 7.55-7.51 (m, 1H), 7.40 (d, 1H), 7.03 (d, 1H), 5.58 (br s, 1H), 3.99 (br s, 1H), 2.43-1.97 (m, 6H), 1.58-1.50 (m, 2H). LC-MS: m/z 552.2 [M+H]⁺.

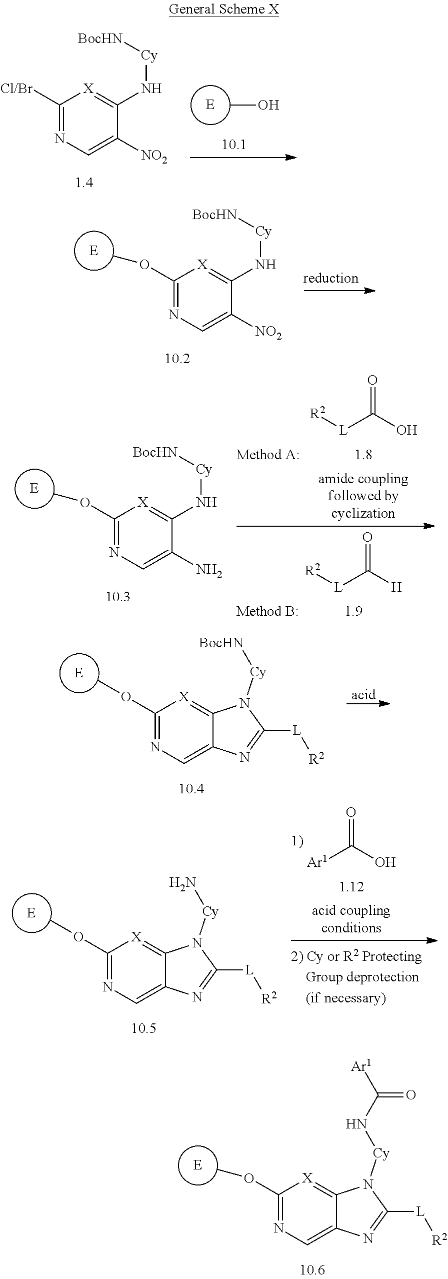

Compounds of formula 10.6 were obtained through General Scheme X. Beginning with a compound of formula 1.4 (prepared via General Scheme I), the chloride/bromide moiety of a compound of formula 1.4 was displaced by the OH group of a compound of formula 10.1 (wherein ring E represents an optionally substituted heterocycloalkyl ring, an optionally substituted heteroaryl ring, or an optionally substituted aryl) in the presence of a suitable base such as cesium carbonate and/or a metal catalyst such as CuI or CuCl to afford compounds of formula 10.2. The nitro group in a compound of formula 10.2 was reduced using a reducing reagent such as iron in the presence of ammonium chloride or Pd/C to afford the pyridine-diamine compounds of formula 10.3. Subsequently, various L and R² groups were installed by coupling the pyridine-diamine compound of formula 10.3 with a L-R² carboxylic acid of formula 1.8 to form an amide intermediate (Method A), which was then cyclized under either basic or acidic conditions to form a compound of formula 10.4. Alternatively, the pyridine-diamine compound of formula 10.3 was condensed with an aldehyde of formula 1.9 (Method B), followed by cyclization and in situ oxidation to afford a compound of formula 10.4. The Boc protecting group of a compound of formula 10.4 was then removed under acidic conditions such as TFA or HCl/dioxanes to form a compound of formula 10.5. Ar¹ groups were then installed by reacting an Ar¹ carboxylic acid of formula 1.12 or a basic salt (i.e., Li, K, or Na) thereof with the amine of formula 10.5 using acid coupling conditions known in the art, such as using one of the following reagents—HATU, EDCI, HOBt—along with a base, such as DIPEA (Hunig's base), pyridine, or TEA. If the compounds still contain any protecting groups on the Cy or R² groups, they may be removed under acidic conditions such as hydrochloric acid to afford compounds of formula 10.6.

### Preparation of Examples 266 and 267 Via General Scheme X

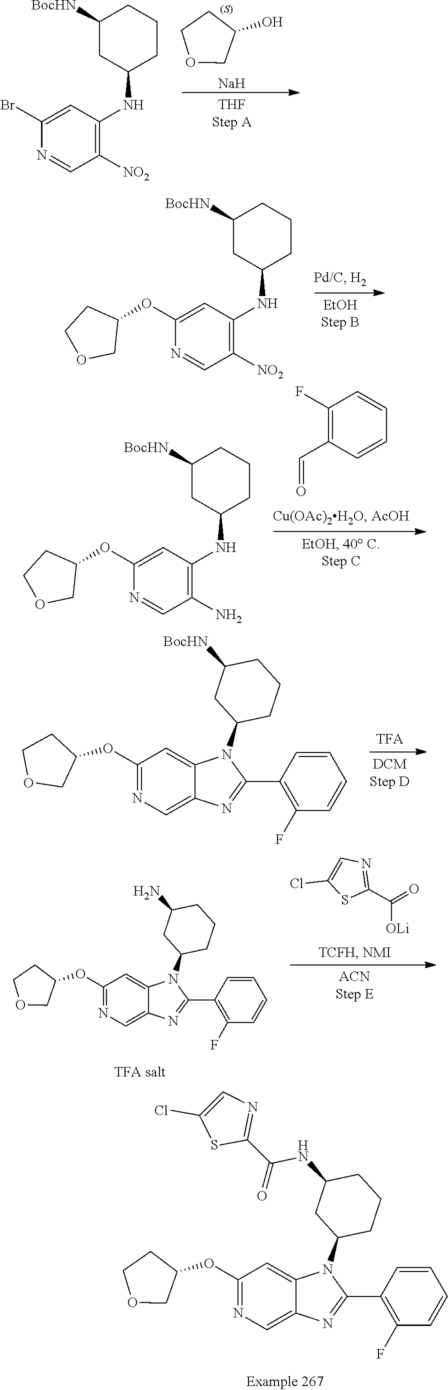

### Step B. Synthesis of tert-butyl ((1S,3R)-3-((5-amino-2-(((S)-tetrahydrofuran-3-yl)oxy)pyridin-4-yl)amino)cyclohexyl)carbamate

To a solution of tert-butyl ((1S,3R)-3-((5-nitro-2-(((S)-tetrahydrofuran-3-yl)oxy)pyridin-4-yl)amino)cyclohexyl)carbamate (1.0 g, 2.4 mmol) in EtOH (35 mL) was added Pd/C (0.2 g, 2.0 mmol, 0.8 eq.) under N₂. The reaction mixture was degassed under vacuum and purged with H₂ several times and then stirred at rt under H₂ atmosphere for 20 hrs. The reaction mixture was filtered through a pad of Celite, and the pad of Celite was washed with EtOH (20 mL). The filtrate was concentrated to dryness to give tert-butyl ((1S,3R)-3-((5-amino-2-(((S)-tetrahydrofuran-3-yl)oxy)pyridin-4-yl)amino)cyclohexyl)carbamate (0.88 g, 90% yield), which was used without further purification. LC-MS: m/z 393.4 [M+H]⁺.

### Step C. Synthesis of tert-butyl ((1S,3R)-3-(2-(2-fluorophenyl)-6-(((S)-tetrahydrofuran-3-yl)oxy)-1H-imidazo[4,5-c]pyridin-1-yl)cyclohexyl)carbamate

To a solution of tert-butyl ((1S,3R)-3-((5-amino-2-(((S)-tetrahydrofuran-3-yl)oxy)pyridin-4-yl)amino)cyclohexyl)carbamate (180 mg, 459 μmol) and 2-fluorobenzaldehyde (85.4 mg, 688 μmol, 1.5 eq.) in EtOH (8 mL) were added Cu(OAc)₂ monohydrate (18.3 mg, 91.7 μmol) and AcOH (13.8 mg, 229 μmol, 0.5 eq.). The reaction mixture was heated to 40° C. and stirred for 20 hrs under N₂ atmosphere. Water (10 mL) was added, and the aqueous portion extracted with DCM (10 mL×3). The combined organic layers were washed with brine (10 mL), dried over Na₂SO₄, filtered, and concentrated to dryness. The residue was purified by C18 column chromatography to afford tert-butyl ((1S,3R)-3-(2-(2-fluorophenyl)-6-(((S)-tetrahydrofuran-3-yl)oxy)-1H-imidazo[4,5-c]pyridin-1-yl)cyclohexyl)carbamate (30 mg, 13% yield). LC-MS: m/z 497.2 [M+H]⁺.

### Step D. Synthesis of (1S,3R)-3-(2-(2-fluorophenyl)-6-(((S)-tetrahydrofuran-3-yl)oxy)-1H-imidazo[4,5-c]pyridin-1-yl)cyclohexan-1-amine

To a solution of tert-butyl ((1S,3R)-3-(2-(2-fluorophenyl)-6-(((S)-tetrahydrofuran-3-yl)oxy)-1H-imidazo[4,5-c]pyridin-1-yl)cyclohexyl)carbamate (25 mg, 50 μmol) in DCM (3 mL) was added TFA (0.3 mL). The reaction mixture was stirred at rt for 2 hrs under N₂ atmosphere and concentrated to dryness to afford (1S,3R)-3-(2-(2-fluorophenyl)-6-(((S)-tetrahydrofuran-3-yl)oxy)-1H-imidazo[4,5-c]pyridin-1-yl)cyclohexan-1-amine (TFA salt) (26 mg, quant.), which was used without further purification. LC-MS: m/z 397.2 [M+H]⁺

### Step E. Synthesis of 5-chloro-N-((1S,3R)-3-(2-(2-fluorophenyl)-6-(((S)-tetrahydrofuran-3-yl)oxy)-1H-imidazo[4,5-c]pyridin-1-yl)cyclohexyl)thiazole-2-carboxamide Example 267

To a solution of (1S,3R)-3-(2-(2-fluorophenyl)-6-(((S)-tetrahydrofuran-3-yl)oxy)-1H-imidazo[4,5-c]pyridin-1-yl)cyclohexan-1-amine (20 mg, 50 μmol) and lithium 5-chlorothiazole-2-carboxylate (8.6 mg, 50 μmol) in ACN (4 mL) was added 1-methylimidazole (8.3 mg, 0.10 mmol, 2 eq.) followed by N,N,N′,N′-tetramethylchloroformamidinium-hexafluorophosphate (TCFH) (21 mg, 76 μmol, 1.5 eq.). The reaction mixture was stirred at rt for 3 hrs under N₂ atmosphere and was then concentrated to dryness. The residue was purified by prep-HPLC to afford Example 267 (18 mg, 64% yield) (TFA salt). ¹H NMR (400 MHz, DMSO-d₆) δ 8.99 (d, 1H), 8.63 (s, 1H), 8.09 (s, 1H), 7.75-7.62 (m, 2H), 7.53-7.40 (m, 2H), 7.13 (s, 1H), 5.65-5.60 (m, 1H), 4.10-3.97 (m, 1H), 3.96-3.82 (m, 3H), 3.81-3.75 (m, 2H), 2.62-2.49 (m, 1H), 2.32-2.23 (m, 1H), 2.10-1.92 (m, 4H), 1.80-1.70 (m, 4H); LC-MS: m/z 542.2 [M+H]⁺; 99% ee.

Example 266 (5-chloro-N-((1S,3R)-3-(2-(2-fluorophenyl)-6-(((R)-tetrahydrofuran-3-yl)oxy)-1H-imidazo[4,5-c]pyridin-1-yl)cyclohexyl)thiazole-2-carboxamide) was prepared according to the above procedures (Step A through Step E), using (R)-tetrahydrofuran-3-ol in Step A. ¹H NMR (400 MHz, DMSO-d₆) δ 8.89 (d, 1H), 8.63 (s, 1H), 8.06 (s, 1H), 7.76-7.61 (m, 2H), 7.55-7.40 (m, 2H), 7.13 (s, 1H), 5.66-5.56 (m, 1H), 4.15-4.02 (m, 1H), 4.01-3.86 (m, 3H), 3.86-3.75 (m, 2H), 2.70-2.55 (m, 1H), 2.35-2.22 (m, 1H), 2.11-1.89 (m, 3H), 1.87-1.72 (m, 3H), 1.64-1.48 (m, 1H), 1.43-1.27 (m, 1H); LC-MS: m/z 542.2 [M+H]⁺; 98% ee.

General Scheme X set forth above was used to synthesize the following compounds by using appropriate starting materials:

| Ex. No. | Structure | ¹H NMR | LC-MS |
|---|---|---|---|
| 268 | 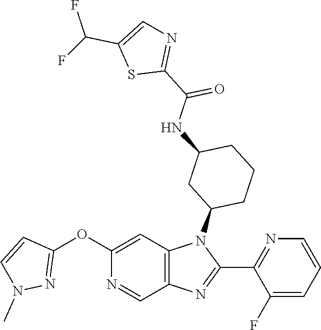 [SMILES: Cn1ccc(Oc2cc3c(cn2)nc(-c2ncccc2F)n3[C@@H]2CCC[C@H](NC(=O)c3ncc(C(F)F)s3)C2)n1] | (400 MHz, CD₃OD) δ 8.69-8.68 (m, 1 H), 8.64 (s, 1 H), 8.15 (s, 1 H), 7.95- 7.90 (m, 1 H), 7.74-7.72 (m, 1 H), 7.58-7.54 (m, 2 H), 7.30-7.03 (m, 1 H), 5.97-5.96 (m, 1 H), 4.71-4.65 (m, 1 H), 4.29-4.27 (m, 1 H), 4.02-3.97 (m, 1 H), 3.84 (s, 1 H), 2.46-2.31 (m, 2 H), 2.22-1.99 (m, 4 H), 1.59-1.48 (m, 2 H). | m/z 569.3 [M + H]⁺ |

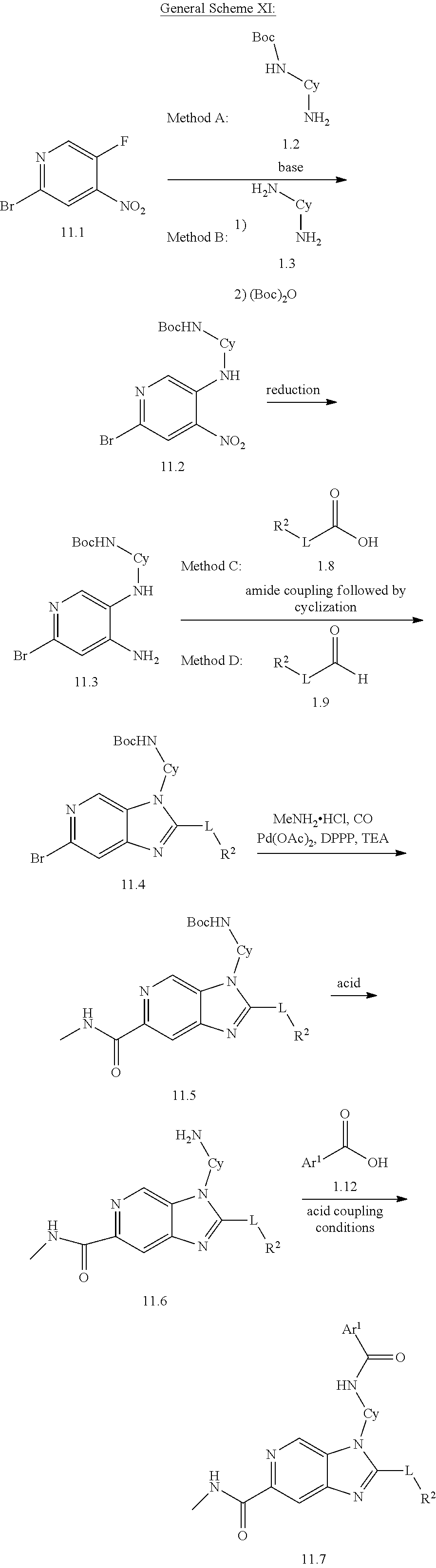

Compounds of formula 11.7 were obtained through General Scheme XI. Beginning with 2-bromo-5-fluoro-4-nitropyridine (reagent 11.1), a nucleophilic aromatic substitution reaction conducted under basic conditions was used to couple a N-Boc-1,3-Cy-diamine compound of formula 1.2 (Method A) to afford compounds of formula 11.2. Alternatively, reagent 11.1 underwent selective nucleophilic aromatic substitution by 1,3-Cy-diamine of formula 1.3 (Method B), and the intermediate amine group was protected with a Boc-protecting group (or other suitable nitrogen protecting group) to afford compounds of formula 11.2. The nitro group in a compound of formula 11.2 was reduced with a reducing agent, such as iron in the presence of ammonium chloride or Pd—C, to afford the pyridine-diamine compound of formula 11.3. Subsequently, various L and R² groups were installed by coupling the pyridine-diamine compound of formula 11.3 with an acid of formula 1.8 to form an amide intermediate (Method C), which was then cyclized under either basic or acidic conditions to form compounds of formula 11.4. Alternatively, the pyridine-diamine compound of formula 11.3 was condensed with an aldehyde of formula 1.9 (Method D), followed by cyclization and in situ oxidation to afford compounds of formula 11.4. The bromide moiety of a compound of formula 11.4 was converted to a N-methyl carboxamide under palladium-catalyzed carbonylation conditions in the presence of methyl amine hydrochloride to afford compounds of formula 11.5. The Boc protecting group of a compound of formula 11.5 was then removed under acidic conditions to form compounds of formula 11.6. Ar¹ groups were then installed by reacting an Ar¹ carboxylic acid of formula 1.12 or a basic salt (i.e., Li, K, or Na) thereof with the amine of formula 11.6 using acid coupling conditions known in the art, such as using one of the following reagents—HATU, EDCI, HOBt—along with a base, such as DIPEA (Hunig's base) or TEA to afford compounds of formula 11.7.

Similarly, compounds of formula 11.8,

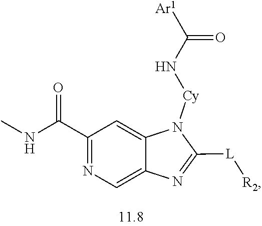

can be prepared following the reactions set forth in General Scheme XI starting from 2-bromo-4-fluoro-5-nitropyridine.

General Scheme XI set forth above was used to synthesize the following compounds by using appropriate starting materials:

| Ex. | | | |
|---|---|---|---|
| No. | Structure | ¹H NMR | LC-MS and % ee |
| 279 | 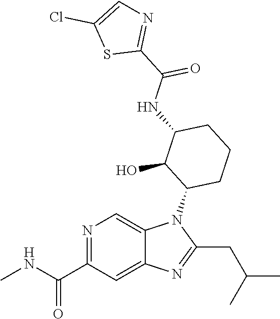 [SMILES: CNC(=O)c1cc2nc(CC(C)C)n([C@H]3CCC[C@@H](NC(=O)c4ncc(Cl)s4)[C@@H]3O)c2cn1] | (400 MHz, CDCl₃) δ 8.74 (s, 1 H), 8.06 (s, 1 H), 8.01-8.00 (m, 1 H), 7.67- 7.65 (m, 1 H), 7.60 (s, 1 H), 4.92 (br s, 1 H), 4.39- 4.27 (m, 2 H), 4.16- 4.09 (m, 1 H), 2.99-2.97 (m, 3 H), 2.89-2.79 (m, 2 H), 2.36-2.24 (m, 2 H), 2.23-2.19 (m, 1 H), 2.06-2.02 (m, 2 H), 1.70- 1.64 (m, 2 H), 1.04- 0.97 (m, 6 H). | m/z 491.2 [M + H]⁺; (column: DAICEL CHIRALCEL OJ (250 mm*30 mm, 10 um); mobile phase: [0.1% NH₃H₂O EtOH]; B %: 30%-30%); Rt = 3.575 min 100% ee |
| 280 | 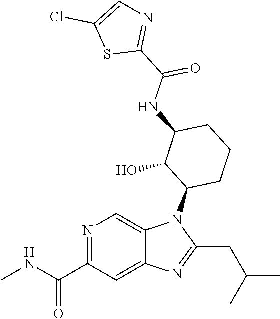 [SMILES: CNC(=O)c1cc2nc(CC(C)C)n([C@@H]3CCC[C@H](NC(=O)c4ncc(Cl)s4)[C@H]3O)c2cn1] | (400 MHz, CDCl₃) δ 8.74 (s, 1 H), 8.07 (s, 1 H), 8.01-8.00 (m, 1 H), 7.66- 7.64 (m, 1 H), 7.60 (s, 1 H), 4.90 (br s, 1 H), 4.39- 4.30 (m, 2 H), 4.13- 4.11 (m, 1 H), 2.99-2.98 (m, 3 H), 2.89-2.79 (m, 2 H), 2.36-2.24 (m, 2 H), 2.23-2.19 (m, 1 H), 2.06-2.02 (m, 2 H), 1.70- 1.64 (m, 2 H), 1.04- 0.97 (m, 6 H). | m/z 491.2 [M + H]⁺; (column: DAICEL CHIRALCEL OJ (250 mm*30 mm, 10 um); mobile phase: [0.1% NH₃H₂O EtOH]; B %: 30%-30%); Rt = 4.070 min 98.5% ee |
| 281 | 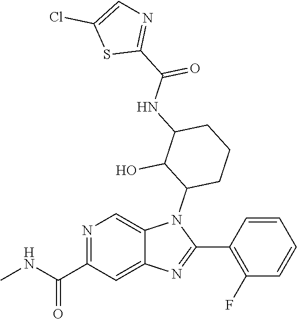 [SMILES: CNC(=O)c1cc2nc(-c3ccccc3F)n(C3CCCC(NC(=O)c4ncc(Cl)s4)C3O)c2cn1] | (400 MHz, CDCl₃) δ 8.86 (s, 1 H), 8.38 (s, 1 H), 8.02 (br s, 1 H), 7.76- 7.74 (m, 1 H), 7.59-7.55 (m, 2 H), 7.41-7.39 (m, 1 H), 7.37-7.35 (m, 1 H), 7.23-7.21 (m, 1 H), 4.27-4.23 (m, 1 H), 4.18- 4.14 (m, 1 H), 4.07 (br s, 1 H), 3.89-3.87 (m, 1 H), 3.00 (br s, 3 H), 2.36- 2.32 (m, 2 H), 2.22- 2.20 (m, 1 H), 2.04-2.01 (m, 1 H), 1.71-1.66 (m, 1 H), 1.58-1.55 (m, 1 H). | m/z 529.2 [M + H]⁺ |
| | (relative stereochemistry at | | |
| | centers 1 and 3 of the cyclohexyl | | |
| | ring is cis with —OH at center 2 | | |
| | trans) | | |

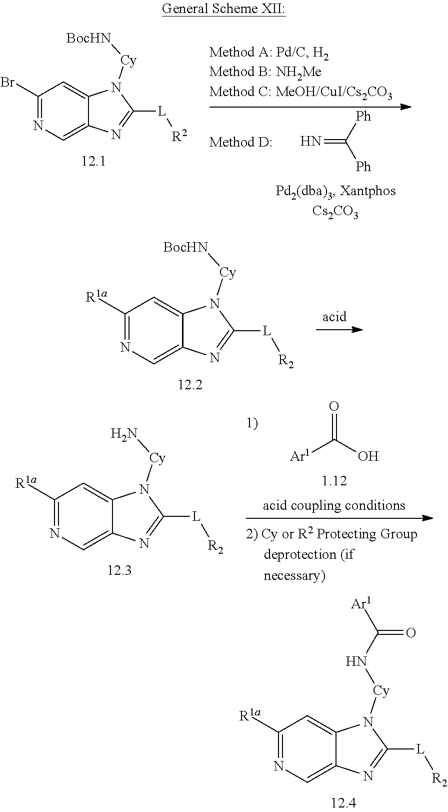

Compounds of formula 12.4 were obtained through the scheme depicted as General Scheme XII. R^{1a} groups were installed by reacting the bromide in compounds of formula 12.1 with the reagents specified in Methods A, B, C, and D to afford compounds of formula 12.2. Under Method A, the bromide of formula 12.1 was cleaved via hydrogenation with Pd/C and H₂ gas to afford compounds of formula 12.2 wherein R^{1a} is hydrogen. Under Method B, the bromide of formula 12.1 was displaced by NH₂CH₃ to afford compounds of formula 12.2 wherein R^{1a} is NHCH₃. Under Method C, the bromide of formula 12.1 was converted to methoxy group via reaction with methanol and a metal catalyst such as CuI and a base such as Cs₂CO₃. Under Method D, the bromide of formula 12.1 was converted to an amine using Buchwald-type coupling conditions. The Boc protecting group in compounds of formula 12.2 was removed under acidic conditions to afford compounds of formula 12.3. Ar¹groups were then installed by reacting an Ar¹ carboxylic acid of formula 1.12 or a basic salt (i.e., Li, K, or Na) thereof with the amine of formula 12.3 using acid coupling conditions known in the art, such as using one of the following reagents—HATU, EDCI, HOBt—along with a base, such as DIPEA (Hunig's base) or TEA to afford compounds of formula 12.4. If compounds of formula 12.4 still contain any protecting groups on the Cy or R² groups, they may be removed under acidic conditions such as hydrochloric acid.

General Scheme XII set forth above was used to synthesize the following compounds by using appropriate starting materials. Examples 275 and 276 were made starting with commercially available 2,4-difluoro-5-nitropyridine.

| Ex. | | | |
|---|---|---|---|
| No. | Structure | ¹H NMR | LC-MS and % ee |
| 269 | 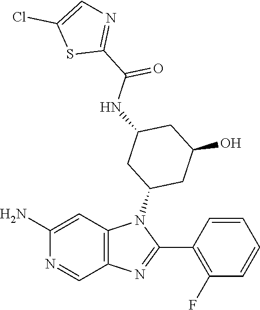 [SMILES: Nc1cc2c(cn1)nc(-c1ccccc1F)n2[C@H]1C[C@H](O)C[C@@H](NC(=O)c2ncc(Cl)s2)C1] | (400 MHz, CD₃OD) δ 8.31 (s, 1 H), 7.71 (s, 1 H), 7.68-7.52 (m, 2 H), 7.34-7.26 (m, 2 H), 6.81 (s, 1 H), 4.58-4.52 (m, 1 H), 4.28-4.22 (m, 2 H), 2.32-2.26 (m, 2 H), 2.13- 2.10 (m, 1 H), 1.98-1.89 (m, 2 H), 1.71-1.64 (m, 1 H). | m/z 487.1 [M + H]⁺; (column: DAICEL CHIRALCEL OD (250 mm*30 mm, 10 um); mobile phase: [0.1% NH₃H₂O EtOH]; B %: 35%-35%); Rt = 3.599 min 99.7% ee |
| 270 | 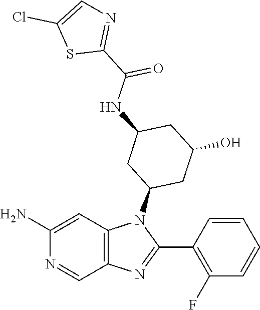 [SMILES: Nc1cc2c(cn1)nc(-c1ccccc1F)n2[C@@H]1C[C@@H](O)C[C@H](NC(=O)c2ncc(Cl)s2)C1] | (400 MHz, CD₃OD) δ 8.30 (s, 1 H), 7.71 (s, 1 H), 7.67-7.50 (m, 2 H), 7.34-7.27 (m, 2 H), 6.79 (s, 1 H), 4.58-4.52 (m, 1 H), 4.28-4.21 (m, 2 H), 2.33-2.26 (m, 2 H), 2.12- 2.10 (m, 1 H), 1.98-1.88 (m, 2 H), 1.71-1.64 (m, 1 H). | m/z 487.1 [M + H]⁺; (column: DAICEL CHIRALCEL OD (250 mm*30 mm, 10 um); mobile phase: [0.1% NH₃H₂O EtOH]; B %: 35%-35%); Rt = 3.890 min 97.7% ee |
| 271 | 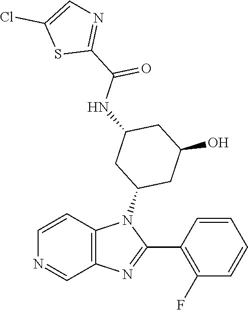 [SMILES: O=C(N[C@@H]1C[C@@H](O)C[C@H](n2c(-c3ccccc3F)nc3cnccc32)C1)c1ncc(Cl)s1] | (400 MHz, CDCl₃) δ 9.17 (s, 1 H), 8.46-8.45 (d, 1 H), 7.67-7.64 (m, 3 H), 7.53-7.52 (m, 1 H), 7.40- 7.38 (m, 1 H), 7.32-7.30 (m, 1 H), 7.09-7.07 (m, 1 H), 4.84-4.83 (m, 1 H), 4.51-4.49 (m, 1 H), 2.43- 2.22 (m, 5 H), 1.74-1.67 (m, 1 H). | m/z 472.1 [M + H]⁺; (column: DAICEL CHIRALCEL OJ (250 mm*30 mm, 10 um); mobile phase: [0.1% NH₃H₂O EtOH]; B %: 25%-25%); Rt = 3.746 min 100% ee |
| 272 | 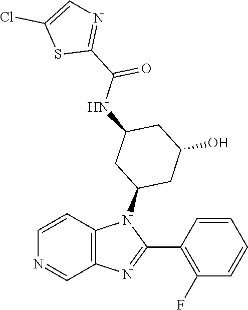 [SMILES: O=C(N[C@H]1C[C@H](O)C[C@@H](n2c(-c3ccccc3F)nc3cnccc32)C1)c1ncc(Cl)s1] | (400 MHz, CDCl₃) δ 9.18 (s, 1 H), 8.47-8.46 (d, 1 H), 7.67-7.64 (m, 3 H), 7.53-7.52 (m, 1 H), 7.40- 7.38 (m, 1 H), 7.32-7.30 (m, 1 H), 7.09-7.07 (m, 1 H), 4.84-4.83 (m, 1 H), 4.51-4.49 (m, 1 H), 2.43- 2.22 (m, 5 H), 1.74-1.67 (m, 1 H). | m/z 472.1 [M + H]⁺; (column: DAICEL CHIRALCEL OJ (250 mm*30 mm, 10 um); mobile phase: [0.1% NH₃H₂O EtOH]; B %: 25%-25%); Rt = 4.160 min 97.2% ee |
| 273 | 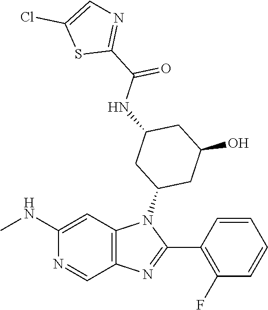 [SMILES: CNc1cc2c(cn1)nc(-c1ccccc1F)n2[C@H]1C[C@H](O)C[C@@H](NC(=O)c2ncc(Cl)s2)C1] | (400 MHz, CDCl₃) δ 8.64 (s, 1 H), 7.66-7.54 (m, 3 H), 7.36-7.32 (m, 1 H), 7.28-7.26 (m, 1 H), 7.09- 7.07 (m, 1 H), 6.34 (s, 1 H), 4.98-4.97 (m, 1 H), 4.76-4.69 (m, 1 H), 4.51- 4.47 (m, 2 H), 3.02 (s, 3 H), 2.39-2.16 (m, 5 H), 1.67-1.61 (m, 1 H). | m/z 501.2 [M + H]⁺; (column: DAICEL CHIRALPAK AD(250 mm*30 mm, 10 um); mobile phase: [0.1% NH₃H₂O IPA]; B %: 40%- 40%); Rt = 4.476 min 100% ee |
| 274 | 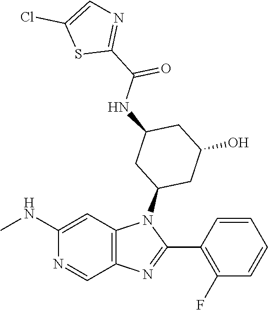 [SMILES: CNc1cc2c(cn1)nc(-c1ccccc1F)n2[C@@H]1C[C@@H](O)C[C@H](NC(=O)c2ncc(Cl)s2)C1] | (400 MHz, CDCl₃) δ 8.64 (s, 1 H), 7.64-7.54 (m, 3 H), 7.36-7.32 (m, 1 H), 7.28-7.26 (m, 1 H), 7.09- 7.07 (m, 1 H), 6.34 (s, 1 H), 4.98-4.97 (m, 1 H), 4.75-4.69 (m, 1 H), 4.48- 4.45 (m, 2 H), 3.01 (s, 3 H), 2.41-2.16 (m, 5 H), 1.67-1.61 (m, 1 H). | m/z 501.2 [M + H]⁺; (column: DAICEL CHIRALPAK AD(250 mm*30 mm, 10 um); mobile phase: [0.1% NH₃H₂O IPA]; B %: 40%- 40%); Rt = 4.824 min 98.1% ee |
| 275 | 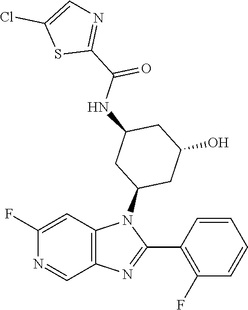 [SMILES: O=C(N[C@H]1C[C@H](O)C[C@@H](n2c(-c3ccccc3F)nc3cnc(F)cc32)C1)c1ncc(Cl)s1] | (400 MHz, CDCl₃) δ 8.73 (s, 1 H), 7.68-7.62 (m, 3 H), 7.41-7.39 (m, 1 H), 7.33-7.30 (m, 1 H), 7.11- 7.07 (m, 2 H), 4.84-4.77 (m, 1 H), 4.51-4.48 (m, 2 H), 2.42-2.21 (m, 5 H), 1.72-1.66 (m, 1 H). | m/z 490.1 [M + H]⁺; (column: DAICEL CHIRALCEL OJ(250 mm*30 mm, 10 um); mobile phase: [0.1% NH₃H₂O EtOH]; B %: 30%-30%); Rt = 2.997 min 100% ee |
| 276 | 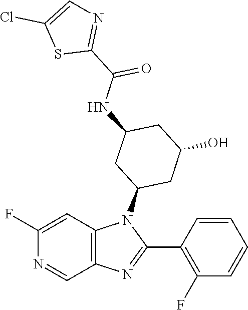 [SMILES: O=C(N[C@H]1C[C@H](O)C[C@@H](n2c(-c3ccccc3F)nc3cnc(F)cc32)C1)c1ncc(Cl)s1] | (400 MHz, CDCl₃) δ 8.73 (s, 1 H), 7.68-7.62 (m, 3 H), 7.41-7.39 (m, 1 H), 7.32-7.28 (m, 1 H), 7.12- 7.08 (m, 2 H), 4.83-4.77 (m, 1 H), 4.51-4.48 (m, 2 H), 2.42-2.24 (m, 5 H), 1.72-1.66 (m, 1 H). | m/z 490.1 [M + H]⁺; (column: DAICEL CHIRALCEL OJ(250 mm*30 mm, 10 um); mobile phase: [0.1% NH₃H₂O EtOH]; B %: 30%-30%); Rt = 3.466 min 98.1% ee |
| 277 | 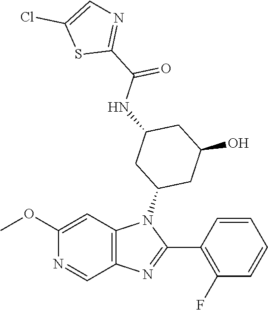 [SMILES: COc1cc2c(cn1)nc(-c1ccccc1F)n2[C@H]1C[C@H](O)C[C@@H](NC(=O)c2ncc(Cl)s2)C1] | (400 MHz, CD₃OD) δ 8.57 (s, 1 H), 7.81 (s, 1 H), 7.72-7.62 (m, 2 H), 7.46-7.37 (m, 2 H), 7.15 (s, 1 H), 4.73-4.67 (m, 1 H), 4.38-4.35 (m, 1 H), 4.31 (br s, 1 H), 4.00 (s, 3 H), 2.49-2.37 (m, 2 H), 2.22-2.19 (m, 1 H), 2.06- 1.97 (m, 2 H), 1.85-1.76 (m, 1 H). | m/z 502.1 [M + H]⁺; (column: DAICEL CHIRALPAK AD (250 mm*30 mm, 10 um); mobile phase: [0.1% NH₃H₂O EtOH]; B %: 30%-30%); Rt = 1.402 min 100% ee |
| 278 | 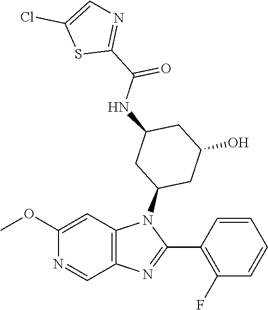 [SMILES: COc1cc2c(cn1)nc(-c1ccccc1F)n2[C@@H]1C[C@@H](O)C[C@H](NC(=O)c2ncc(Cl)s2)C1] | (400 MHz, CD₃OD) δ 8.57 (s, 1 H), 7.81 (s, 1 H), 7.72-7.62 (m, 2 H), 7.46-7.37 (m, 2 H), 7.15 (s, 1 H), 4.73-4.67 (m, 1 H), 4.43-4.34 (m, 1 H), 4.31 (br s, 1 H), 4.00 (s, 3 H), 2.48-2.40 (m, 2 H), 2.22-2.19 (m, 1 H), 2.05- 1.97 (m, 2 H), 1.84- 1.78 (m, 1 H). | m/z 502.1 [M + H]⁺; (column: DAICEL CHIRALPAK AD (250 mm*30 mm, 10 um); mobile phase: [0.1% NH₃H₂O EtOH]; B %: 30%-30%); Rt = 1.402 min 98.59% ee |

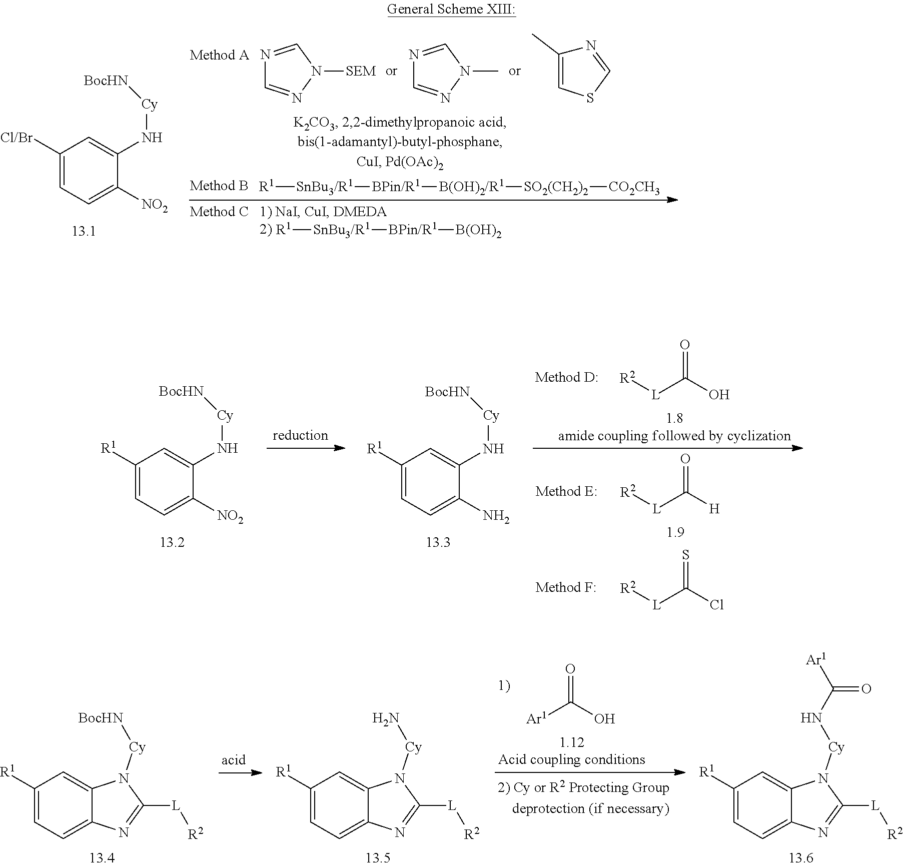

Compounds of formula 13.6 were obtained through the scheme depicted as General Scheme XIII. The chloride/bromide moiety of a compound of formula 13.1 can be used as a functional handle to install various R¹ aryls, R¹ heteroaryls, and R¹ heterocycloalkyls. Under Method A, unsubstituted and substituted 1,2,4-triazoles, thiazoles, and oxazoles can be installed via a C—H functionalization reaction of the heteroaryl. Under Method B, the chloride/bromide moiety of a compound of formula 13.1 was converted to a R¹ heteroaryl or aryl using a Stille coupling (R¹—SnBu₃), Suzuki coupling (R¹-BPin or R¹—B(OH)₂), or a cross-coupling with a substituted sulfone (R¹—SO₂(CH₂)₂—CO₂CH₃) to afford compounds of formula 13.2. In some cases, the chloride/bromide moiety needed to be converted to the more reactive iodide moiety using the reaction conditions in Method C prior to the Stille or Suzuki coupling. The nitro group in a compound of formula 13.2 was reduced using a reducing reagent such as iron in the presence of ammonium chloride or Pd/C to afford the pyridine-diamine compounds of formula 13.3. Subsequently, various L and R² groups were installed by coupling the pyridine-diamine compound of formula 13.3 with a L-R² carboxylic acid of formula 1.8 to form an amide intermediate (Method D), which was then cyclized under either basic or acidic conditions to form a compound of formula 13.4. Alternatively, the pyridine-diamine compound of formula 13.3 was condensed with an aldehyde of formula 1.9 (Method E), followed by cyclization and in situ oxidation to afford a compound of formula 13.4. Alternatively, as shown in Method F, the pyridine-diamine compound of formula 13.3 was condensed with a carbothioyl chloride to form an amide intermediate, which was then cyclized under basic or acidic conditions to form a compound of formula 13.4. The Boc protecting group of a compound of formula 13.4 was then removed under acidic conditions such as TFA or HCl/dioxanes to form a compound of formula 13.5. Ar¹ groups were then installed by reacting an Ar¹ carboxylic acid of formula 1.12 or a basic salt (i.e., Li, K, or Na) thereof with the amine of formula 13.5 using acid coupling conditions known in the art, such as using one of the following reagents—HATU, EDCI, HOBt—along with a base, such as DIPEA (Hunig's base), pyridine, or TEA. If the compounds still contain any protecting groups on the Cy or R² groups, they may be removed under acidic conditions such as hydrochloric acid to afford compounds of formula 13.6.

### Preparation of Example 466 Via General Scheme XIII

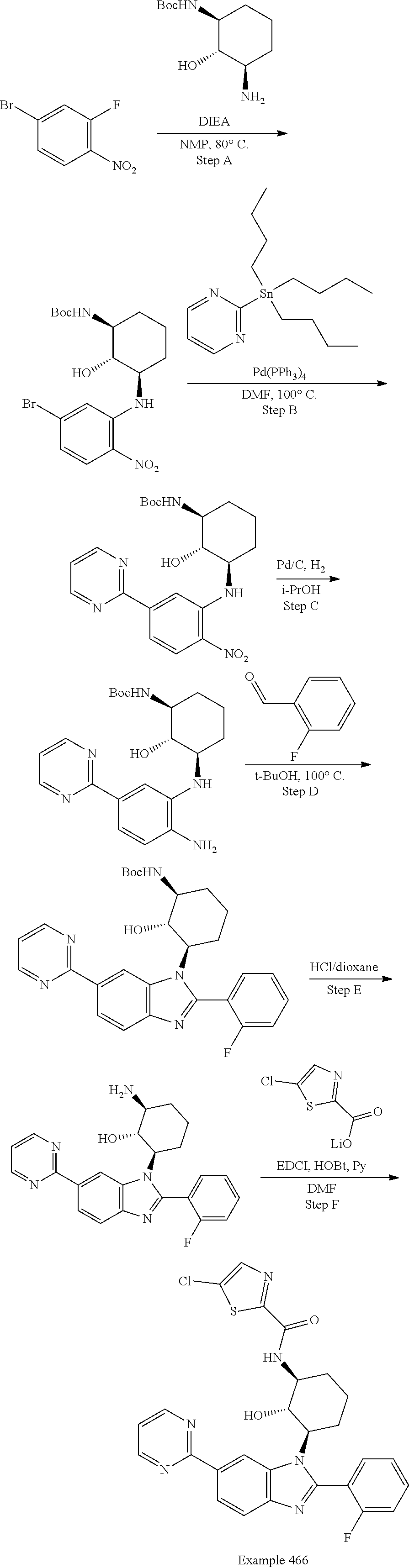

### Step A. Synthesis of tert-butyl ((1S,2S,3R)-3-((5-bromo-2-nitrophenyl)amino)-2-hydroxycyclohexyl)carbamate

A mixture of 4-bromo-2-fluoro-1-nitrobenzene (3.0 g, 14 mmol), tert-butyl ((1S,2S,3R)-3-amino-2-hydroxycyclohexyl)carbamate (3.5 g, 15 mmol) and DIEA (3.5 g, 4.8 mL, 27 mmol) in NMP (30 mL) was stirred at 80° C. for 2 hrs. The reaction mixture was poured into ice-water (20 mL), and the aqueous portion extracted with EtOAc (20 mL×3). The combined organic layers were washed with brine (10 mL), dried over Na₂SO₄, filtered, and concentrated to dryness. The residue was purified by flash column chromatography on silica gel to give tert-butyl ((1S,2S,3R)-3-((5-bromo-2-nitrophenyl)amino)-2-hydroxycyclohexyl)carbamate (5.28 g, 90% yield). LC-MS: m/z 430 [M+H]⁺.

### Step B. Synthesis of tert-butyl ((1S,2S,3R)-2-hydroxy-3-((2-nitro-5-(pyrimidin-2-yl)phenyl)amino)cyclohexyl)carbamate

To a solution of tert-butyl ((1S,2S,3R)-3-((5-bromo-2-nitrophenyl)amino)-2-hydroxycyclohexyl)carbamate (100 mg, 232 μmol) and 2-(tributylstannyl)pyrimidine (129 mg, 349 μmol) in DMF (5 mL) was added Pd(PPh₃)₄ (27 mg, 23.2 μmol). The resulting mixture was stirred at 100° C. for 16 hrs. Water (10 mL) was added, and the aqueous portion extracted with EtOAc (30 mL×3). The combined organic layers were washed with brine (10 mL), dried over Na₂SO₄, filtered, and concentrated to dryness. The residue was purified by flash column chromatography on silica gel to give tert-butyl ((1S,2S,3R)-2-hydroxy-3-((2-nitro-5-(pyrimidin-2-yl)phenyl)amino)cyclohexyl)carbamate (35 mg, 35% yield). LC-MS: m/z 430 [M+H]⁺.

### Step C. Synthesis of tert-butyl ((1S,2S,3R)-3-((2-amino-5-(pyrimidin-2-yl)phenyl)amino)-2-hydroxycyclohexyl)carbamate

To a solution of tert-butyl ((1S,2S,3R)-2-hydroxy-3-((2-nitro-5-(pyrimidin-2-yl)phenyl)amino)cyclohexyl)carbamate (850 mg, 1.98 mmol) in i-PrOH (5 mL) was added Pd/C (211 mg). The suspension was degassed under vacuum and purged with H₂ several times. The resulting mixture was stirred at rt under H₂ (1 atm) for 6 hrs. The reaction mixture was filtered through a pad of Celite, and the filter cake was washed with MeOH (20 mL). The filtrate was concentrated to dryness to give tert-butyl ((1S,2S,3R)-3-((2-amino-5-(pyrimidin-2-yl)phenyl)amino)-2-hydroxycyclohexyl)carbamate (800 mg), which was used without further purification. LC-MS: m/z 400 [M+H]⁺.

### Step D. Synthesis of tert-butyl ((1S,2R,3R)-3-(2-(2-fluorophenyl)-6-(pyrimidin-2-yl)-1H-benzo[d]imidazol-1-yl)-2-hydroxycyclohexyl)carbamate

A solution of tert-butyl ((1S,2S,3R)-3-((2-amino-5-(pyrimidin-2-yl)phenyl)amino)-2-hydroxycyclohexyl)carbamate (250 mg, 626 mol) and 2-fluorobenzaldehyde (233 mg, 1.88 mmol) in t-BuOH (15 mL) was stirred at 100° C. for 20 hrs and then concentrated to dryness. The residue was purified by flash column chromatography on silica gel to give tert-butyl ((1S,2R,3R)-3-(2-(2-fluorophenyl)-6-(pyrimidin-2-yl)-1H-benzo[d]imidazol-1-yl)-2-hydroxycyclohexyl)carbamate (200 mg, 64% yield). LC-MS: m/z 504 [M+H]⁺.

### Step E. Synthesis of (1R,2S,6R)-2-amino-6-(2-(2-fluorophenyl)-6-(pyrimidin-2-yl)-1H-benzo[d]imidazol-1-yl)cyclohexan-1-ol

A solution of tert-butyl ((1S,2R,3R)-3-(2-(2-fluorophenyl)-6-(pyrimidin-2-yl)-1H-benzo[d]imidazol-1-yl)-2-hydroxycyclohexyl)carbamate (150 mg, 298 mol) in HCl/1,4-dioxane (5 mL, 4M) was stirred at rt for 1 hr and then concentrated to dryness to give (1R,2S,6R)-2-amino-6-(2-(2-fluorophenyl)-6-(pyrimidin-2-yl)-1H-benzo[d]imidazol-1-yl)cyclohexan-1-ol (230 mg, quant.), which was used without further purification. LC-MS: m/z 404 [M+H]⁺.

### Step F. Synthesis of 5-chloro-N-((1S,2R,3R)-3-(2-(2-fluorophenyl)-6-(pyrimidin-2-yl)-1H-benzo[d]imidazol-1-yl)-2-hydroxycyclohexyl)thiazole-2-carboxamide

### Example 466

To a solution of (1R,2S,6R)-2-amino-6-(2-(2-fluorophenyl)-6-(pyrimidin-2-yl)-1H-benzo[d]imidazol-1-yl)cyclohexan-1-ol (50 mg, 0.12 mmol), pyridine (20 mg, 0.25 mmol) and lithium 5-chlorothiazole-2-carboxylate (25 mg, 0.15 mmol) in DMF (2 mL) were added EDCI (36 mg, 0.19 mmol) and HOBt (28 mg, 0.19 mmol). The reaction mixture was stirred at rt for 2 hrs and then purified by prep-HPLC to give Example 466 (12 mg, 17% yield). ¹H NMR (400 MHz, DMSO-d₆) δ 9.02-8.94 (m, 3H), 8.84 (s, 1H), 8.39 (d, 1H), 8.01 (s, 1H), 7.82 (d, 1H), 7.73-7.64 (m, 2H), 7.50-7.40 (m, 3H), 5.09 (s, 1H), 4.43 (t, 1H), 3.99-3.93 (m, 1H), 3.75-3.61 (m, 1H), 2.47-2.37 (m, 1H), 2.18-2.07 (m, 1H), 2.01-1.75 (m, 3H), 1.51-1.35 (m, 1H); LC-MS: m/z 549 [M+H]⁺.

Compounds of formula IVA, IVB, VA, VB, VC, and VD may be obtained in accordance with General Schemes I-XIII using appropriately substituted fluoro-nitrobenzenes.

| Ex. | | | | |
|---|---|---|---|---|
| No. | Structure | ¹H NMR | LC-MS | Starting Reagent |
| 282 | 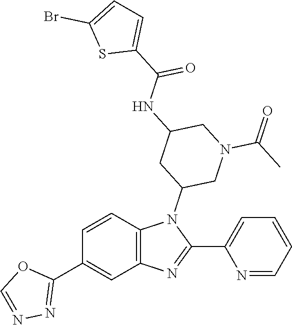 [SMILES: CC(=O)N1CC(NC(=O)c2ccc(Br)s2)CC(n2c(-c3ccccn3)nc3cc(-c4nnco4)ccc32)C1] | (400 MHz, DMSO-d₆) δ 9.33 (s, 1 H), 8.80- 8.72 (m, 1 H), 8.65- 8.55 (m, 1 H), 8.35 (s, 1 H), 8.25-8.17 (m, 2 H), 8.05-7.97 (m, 2 H), 7.57-7.55 (m, 2 H), 7.26-7.25 (m, 1 H), 5.69-5.58 (m, 1 H), 4.65-4.60 (m, 1 H), 4.33-4.30 (m, 1 H), 4.18-3.80 (m, 3 H), 2.77-2.64 (m, 2 H), 2.25-2.17 (m, 1 H), 2.14-2.06 (m, 3 H). | m/z 592.1 [M + H]⁺ | 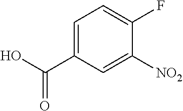 [SMILES: O=C(O)c1ccc(F)c([N+](=O)[O-])c1] |
| | (relative | | | |
| | stereochemistry at | | | |
| | centers 3 and 5 of | | | |
| | piperidine ring is cis) | | | |
| 283 | 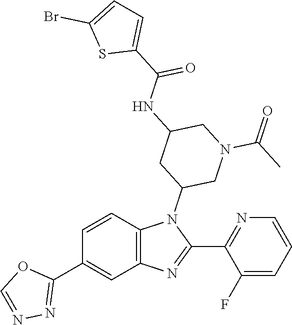 [SMILES: CC(=O)N1CC(NC(=O)c2ccc(Br)s2)CC(n2c(-c3ncccc3F)nc3cc(-c4nnco4)ccc32)C1] | (400 MHz, CD₃OD) δ 9.04 (s, 1 H), 8.67- 8.64 (m, 1 H), 8.47 (s, 1 H), 8.17 (s, 2 H), 7.95-7.88 (m, 2 H), 7.72-7.70 (m, 1 H), 7.50-7.48 (m, 1 H), 7.14-7.12 (m, 1 H), 5.02-4.98 (m, 1 H), 4.85-4.80 (m, 1 H), 4.45-4.40 (m, 0.5 H), 4.18-4.14 (m, 0.5 H), 4.05-3.95 (m, 1.5 H), 3.50-3.40 (m, 1 H), 3.18-3.16 (m, 0.5 H), 2.76-2.70 (m, 1.5 H), 2.40-2.34 (m, 1 H), 2.24-2.18 (m, 3 H). | m/z 610.0 [M + H]⁺ | 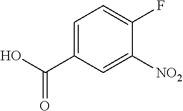 [SMILES: O=C(O)c1ccc(F)c([N+](=O)[O-])c1] |
| | (relative | | | |
| | stereochemistry at | | | |
| | centers 3 and 5 of | | | |
| | piperidine ring is cis) | | | |
| 284 | 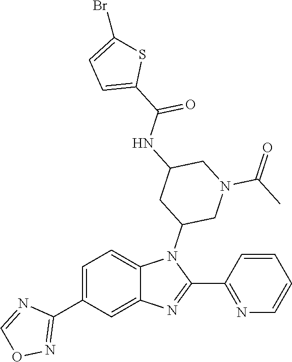 [SMILES: CC(=O)N1CC(NC(=O)c2ccc(Br)s2)CC(n2c(-c3ccccn3)nc3cc(-c4ncon4)ccc32)C1] | (400 MHz, DMSO-d₆) δ 9.73 (s, 1 H), 8.80- 8.70 (m, 1 H), 8.65- 8.55 (m, 1 H), 8.41 (s, 1 H), 8.27-8.18 (m, 2 H), 8.09-8.02 (m, 2 H), 7.63-7.60 (m, 2 H), 7.32-7.29 (m, 1 H), 5.67-5.58 (m, 1 H), 4.65-4.62 (m, 1 H), 4.33-4.30 (m, 1 H), 4.08-3.86 (m, 2 H), 2.79-2.68 (m, 2 H), 2.25-2.22 (m, 1 H), 2.19-2.11 (m, 3 H). | m/z 594.2 [M + H]⁺ | 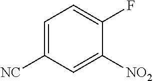 [SMILES: N#Cc1ccc(F)c([N+](=O)[O-])c1] |
| | (relative | | | |
| | stereochemistry at | | | |
| | centers 3 and 5 of | | | |
| | piperidine ring is cis) | | | |
| 285 | 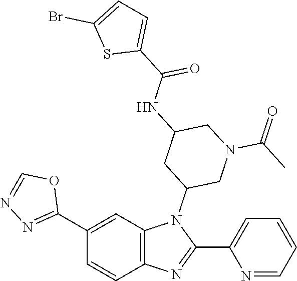 [SMILES: CC(=O)N1CC(NC(=O)c2ccc(Br)s2)CC(n2c(-c3ccccn3)nc3ccc(-c4nnco4)cc32)C1] | (400 MHz, DMSO-d₆) δ 9.39-9.38 (m, 1 H), 8.78-8.64 (m, 2 H), 8.51-8.50 (m, 1 H), 8.25-8.23 (m, 1 H), 8.05-8.03 (m, 1 H), 7.95 (br s, 2 H), 7.60- 7.56 (m, 2 H), 7.26- 7.24 (m, 1 H), 5.64- 5.53 (m, 1 H), 4.61- 4.59 (m, 1 H), 4.29- 4.27 (m, 1 H), 4.04- 3.85 (m, 2 H), 2.74- 2.61 (m, 2 H), 2.35- 2.23 (m, 1 H), 2.13- 2.07 (m, 3 H). | m/z 592.0 [M + H]⁺ | 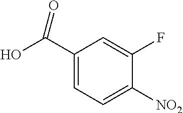 [SMILES: O=C(O)c1ccc([N+](=O)[O-])c(F)c1] |
| | (relative | | | |
| | stereochemistry at | | | |
| | centers 3 and 5 of | | | |
| | piperidine ring is cis) | | | |
| 286 | 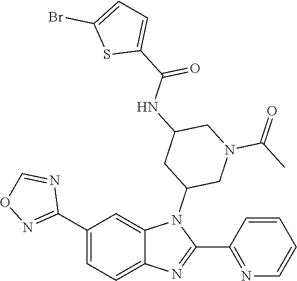 [SMILES: CC(=O)N1CC(NC(=O)c2ccc(Br)s2)CC(n2c(-c3ccccn3)nc3ccc(-c4ncon4)cc32)C1] | (400 MHz, CDCl₃) δ 8.81-8.79 (m, 1 H), 8.68-8.67 (m, 1 H), 8.35-8.28 (m, 2 H), 8.11-8.09 (m, 1 H), 7.94-7.92 (m, 2 H), 7.32-7.24 (m, 1.5 H), 7.22-7.21(m, 0.5 H), 7.05-6.99 (m, 1 H), 6.23 (br s, 0.5 H), 6.23 (br s, 0.5 H), 5.83- 5.81 (m, 1 H), 5.02 (br s, 0.5 H), 4.88 (br s, 0.5 H), 4.37 (br s, 0.5 H), 4.34 (br s, 0.5 H), 4.19-4.17 (m, 1 H), 3.89 (br s, 0.4 H), 3.03 (br s, 0.5 H), 2.71- 2.52 (m, 2.5 H), 1.73 (s, 1 H). | m/z 592.2 [M + H]⁺ | 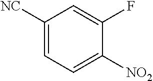 [SMILES: N#Cc1ccc([N+](=O)[O-])c(F)c1] |
| | (relative | | | |
| | stereochemistry at | | | |
| | centers 3 and 5 of | | | |
| | piperidine ring is cis) | | | |
| 287 | 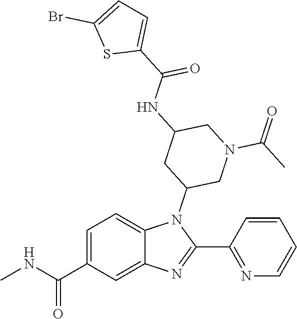 [SMILES: CNC(=O)c1ccc2c(c1)nc(-c1ccccn1)n2C1CC(NC(=O)c2ccc(Br)s2)CN(C(C)=O)C1] | (400 MHz, CD₃OD) δ 8.82-8.79 (m, 1 H), 8.27 (s, 1 H), 8.25- 8.18 (m, 1 H), 8.12- 8.04 (m, 2 H), 7.94- 7.92 (m, 1 H), 7.70- 7.52 (m, 2 H), 7.18- 7.15 (m, 1 H), 5.70- 5.56 (m, 1 H), 4.80- 4.70 (m, 1 H), 4.52- 4.48 (m, 0.5 H), 4.27- 4.23 (m, 0.5 H), 4.10- 4.02 (m, 1.5 H), 3.62- 3.58 (m, 0.5 H), 3.24- 3.21 (m, 0.5 H), 2.99 (s, 3 H), 2.85-2.80 (m, 1.5 H), 2.52-2.48 (m, 0.5 H), 2.38-2.24 (m, 3.5 H). | m/z 581.0 [M + H]⁺ | 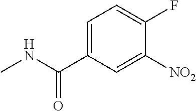 |
| | (relative | | | |
| | stereochemistry at | | | |
| | centers 3 and 5 of | | | |
| | piperidine ring is cis) | | | |
| 288 | 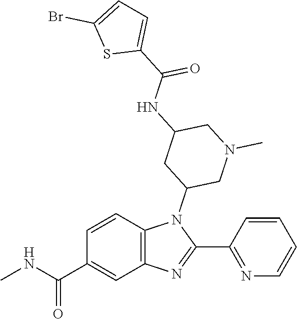 [SMILES: CNC(=O)c1ccc2c(c1)nc(-c1ccccn1)n2C1CC(NC(=O)c2ccc(Br)s2)CN(C)C1] | (400 MHz, CD₃OD) δ 8.85-8.83 (m, 1 H), 8.25 (s, 1 H), 8.17- 8.15 (m, 1 H), 8.07- 7.89 (m, 3 H), 7.62- 7.58 (m, 1 H), 7.52- 7.51 (m, 1 H), 7.17- 7.15 (m, 1 H), 5.77- 5.70 (m, 1 H), 3.22- 3.15 (m, 2 H), 2.99- 2.93 (m, 4 H), 2.59- 2.55 (m, 1 H), 2.44 (s, 3 H), 2.38-2.35 (m, 1 H), 2.23-2.17 (m, 1 H). | m/z 553.0 [M + H]⁺ | 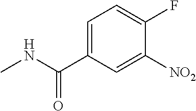 |
| | (relative | | | |
| | stereochemistry at | | | |
| | centers 3 and 5 of | | | |
| | piperidine ring is cis) | | | |
| 289 | 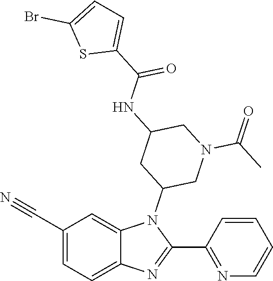 [SMILES: CC(=O)N1CC(NC(=O)c2ccc(Br)s2)CC(n2c(-c3ccccn3)nc3ccc(C#N)cc32)C1] | (400 MHz, CDCl₃) δ 8.71-8.69 (m, 1 H), 8.32-8.28 (m, 1 H), 8.04-7.89 (m, 3 H), 7.56-7.54 (m, 2 H), 7.31 (br s, 1 H), 7.24- 7.20 (m, 1 H), 6.32- 6.25 (m, 0.5 H), 5.88- 5.84 (m, 1 H), 5.04- 4.89 (m, 1 H), 4.66- 4.35 (m, 1 H), 4.33- 4.22 (m, 1 H), 3.80 (br s, 0.5 H), 3.37 (br s, 0.5 H), 3.04 (br s, 0.5 H), 2.71-2.31(m, 2.5 H), 2.21(s, 3 H). | m/z 551.2 [M + H]⁺ | 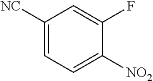 [SMILES: N#Cc1ccc([N+](=O)[O-])c(F)c1] |
| | (relative | | | |
| | stereochemistry at | | | |
| | centers 3 and 5 of | | | |
| | piperidine ring is cis) | | | |
| 290 | 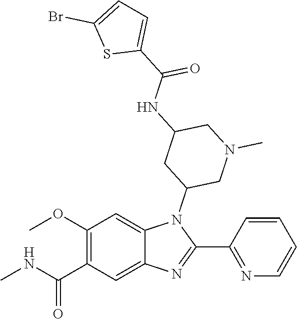 [SMILES: CNC(=O)c1cc2nc(-c3ccccn3)n(C3CC(NC(=O)c4ccc(Br)s4)CN(C)C3)c2cc1OC] | (400 MHz, CD₃OD) δ 8.82-8.81 (m, 1 H), 8.32 (s, 1 H), 8.11- 8.04 (m, 2 H), 7.59- 7.57 (m, 1 H), 7.50- 7.48 (m, 1 H), 7.40 (s, 1 H), 7.17-7.15 (m, 1 H), 5.70-5.63 (m, 1 H), 4.27-4.21 (m, 1 H), 4.15 (s, 3 H), 3.19- 3.15 (m, 2 H), 3.01 (s, 3 H), 2.96-2.91 (m, 1 H), 2.58-2.55 (m, 1 H), 2.44 (s, 3 H), 2.38-2.35 (m, 1 H), 2.23-2.17 (m, 1 H). | m/z 583.3 [M + H]⁺ | 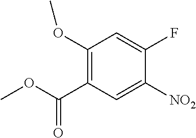 [SMILES: COC(=O)c1cc([N+](=O)[O-])c(F)cc1OC] |
| | (relative | | | |
| | stereochemistry at | | | |
| | centers 3 and 5 of | | | |
| | piperidine ring is cis) | | | |
| 291 | 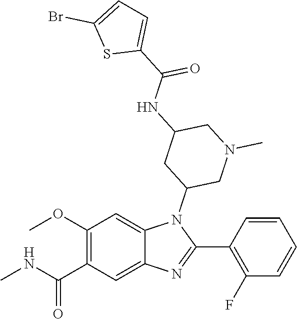 [SMILES: CNC(=O)c1cc2nc(-c3ccccc3F)n(C3CC(NC(=O)c4ccc(Br)s4)CN(C)C3)c2cc1OC] | (400 MHz, CD₃OD) δ 8.27 (s, 1 H), 7.70- 7.64 (m, 2 H), 7.45- 7.34 (m, 4 H), 7.13- 7.12 (m, 1 H), 4.45- 4.42 (m, 1 H), 4.11- 4.07 (m, 4 H), 3.10- 3.01 (m, 2 H), 2.97 (s, 3 H), 2.80-2.76 (m, 1 H), 2.46-2.42 (m, 1 H), 2.36 (s, 3 H), 2.23- 2.20 (m, 1 H), 2.12- 2.08 (m, 1 H). | m/z 600.1 [M + H]⁺ | 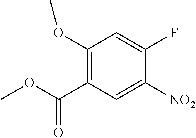 [SMILES: COC(=O)c1cc([N+](=O)[O-])c(F)cc1OC] |
| | (relative | | | |
| | stereochemistry at | | | |
| | centers 3 and 5 of | | | |
| | piperidine ring is cis) | | | |
| 292 | 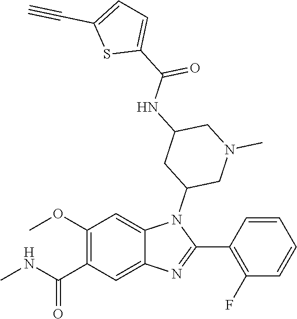 [SMILES: C#Cc1ccc(C(=O)NC2CC(n3c(-c4ccccc4F)nc4cc(C(=O)NC)c(OC)cc43)CN(C)C2)s1] | (400 MHz, CD₃OD) δ 8.27 (s, 1 H), 7.67- 7.64 (m, 2 H), 7.54- 7.53 (m, 1 H), 7.43- 7.35 (m, 3 H), 7.23- 7.22 (m, 1 H), 4.46- 4.44 (m, 1 H), 4.12- 4.09 (m, 4 H), 3.10- 2.97 (m, 6 H), 2.80- 2.77 (m, 1 H), 2.48- 2.42 (m, 1 H), 2.36 (s, 3 H), 2.25-2.20 (m, 1 H), 2.14-2.10 (m, 1 H). | m/z 546.2 [M + H]⁺ | 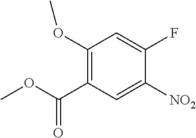 [SMILES: COC(=O)c1cc([N+](=O)[O-])c(F)cc1OC] |
| | (relative | | | |
| | stereochemistry at | | | |
| | centers 3 and 5 of | | | |
| | piperidine ring is cis) | | | |
| 293 | 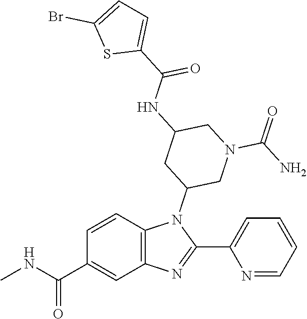 [SMILES: CNC(=O)c1ccc2c(c1)nc(-c1ccccn1)n2C1CC(NC(=O)c2ccc(Br)s2)CN(C(N)=O)C1] | (400 MHz, CD₃OD) δ 8.78-8.77 (m, 1 H), 8.22 (s, 1 H), 8.16- 8.14 (m, 1 H), 8.03- 7.96 (m, 2 H), 7.88- 7.86 (m, 1 H), 7.56- 7.48 (m, 2 H), 7.13- 7.12 (m, 1 H), 5.59- 5.53 (m, 1 H), 4.39- 4.27 (m, 2 H), 4.29- 4.27 (m, 1 H), 4.10- 4.05 (m, 1 H), 3.76- 3.70 (m, 1 H), 2.95- 2.90 (m, 4 H), 2.77- 2.69 (m, 1 H), 2.45- 2.42 (m, 1 H). | m/z 584.0 [M + H]⁺ | 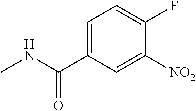 |
| | (relative | | | |
| | stereochemistry at | | | |
| | centers 3 and 5 of | | | |
| | piperidine ring is cis) | | | |
| 294 | 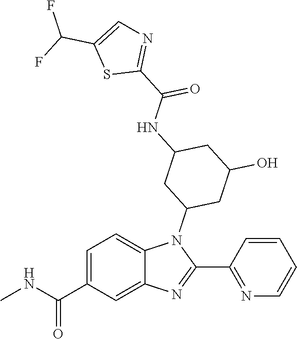 [SMILES: CNC(=O)c1ccc2c(c1)nc(-c1ccccn1)n2C1CC(O)CC(NC(=O)c2ncc(C(F)F)s2)C1] | (400 MHz, CD₃OD) δ 8.86-8.85 (m, 1 H), 8.24-8.23 (m, 1 H), 8.18-8.17 (m, 1 H), 8.11-8.06 (m, 2 H), 7.98-7.96 (m, 1 H), 7.89-7.88 (m, 1 H), 7.60-7.58 (m, 1 H), 7.19 (t, 1 H), 5.87- 5.84 (m, 1 H), 4.57- 4.54 (m, 1 H), 4.40 (br s, 1 H), 2.99 (s, 3 H), 2.65-2.56 (m, 2 H), 2.44-2.41 (m, 1 H), 2.15-2.13 (m, 2 H), 1.93-1.90 (m, 1 H), 2.14-2.10 (m, 1 H). | 527.2 [M + H]⁺ | 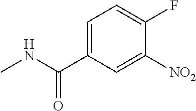 |
| | (relative | | | |
| | stereochemistry at | | | |
| | centers 1 and 5 of | | | |
| | cyclohexyl ring is cis | | | |
| | with —OH at center 3 | | | |
| | trans) | | | |
| 465 | 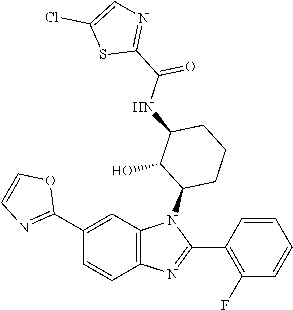 [SMILES: O=C(N[C@H]1CCC[C@@H](n2c(-c3ccccc3F)nc3ccc(-c4ncco4)cc32)[C@@H]1O)c1ncc(Cl)s1] | (400 MHz, DMSO-d₆) δ 8.95 (d, 1H), 8.37 (s, 1H), 8.29 (s, 1H), 8.03 (s, 1H), 7.94 (d, 1H), 7.85 (d, 1H), 7.71-7.64 (m, 2H), 7.49-7.39 (m, 3H), 5.12 (d, 1H), 4.50-4.38 (m, 1H), 4.02-3.91 (m, 1H), 3.75-3.64 (m, 1H), 2.43-2.31 (m, 1H), 2.12-2.00 (m, 1H), 1.90-1.74 (m, 3H), 1.49-1.37 (m, 1H). | m/z 538.3 [M + H]⁺ | 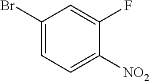 [SMILES: O=[N+]([O-])c1ccc(Br)cc1F] |

### BCAT2 In Vitro BCAT2 Cell Inhibition Using Engineered A549 Cells

Endogenous Methylmalonyl-CoA Mutase (MUT) and Branched Chain Aminotransferase 1 (BCAT1) in A549 cells was knocked out by CRISPR/CAS9 using the targeting system from TransOMIC Technologies. The following guide sequence was cloned into pCLIP-ALL-EFS-Blast vector to target human MUT: ATCATGTAAGAACCTCCCCA. The following guide sequence was cloned into pCLIP-ALL-EFS-Puro vector to target human BCAT1: TACTGGATAAATGAAGATGG. The resulting plasmids were co-transfected into 293T cells with VSV-G, P-PEX, and Fugene-6. Lentivirus was harvested and A549 cells were first transduced with lentivirus targeting MUT and polybrene. After genetic selection with blasticidin clonal selection was performed and validated via western blot analysis to confirm MUT was no longer expressed. These MUT KO cells were then transduced with lentivirus targeting BCAT1 and polybrene. After selection with puromycin clonal selection was performed and clones were validated via western blot analysis to confirm BCAT1 was no longer expressed in these cells. The result was an engineered A549 cell line that expressed endogenous BCAT2.

For KMV cell assays, on day 1, 25,000 cells/100 μL EMEM (10% FBS) were seeded in each well of a 96-well plate. On day 2, medium was replaced with 50 μL fresh EMEM (10% dialyzed FBS) containing 2× the assay concentration of experimental compound. After 15 minutes, 50 μL of 800 uM 15N,13C6-Isoleucine in EMEM (10% dialyzed FBS) was added to each well and incubated for 24 hours. On day 3, 50 μL of media from each well was collected and transferred to another 96-well plate for 13C6-3-Methyl-2-Oxopentanoic Acid (KMV) analysis via LC-MS/MS. In the remaining assay wells 50 μL of Promega Glo CTG reagent was added and luminescence was measured as a cell viability readout.

For LC-MS/MS analysis 5 μL of cell assay media from each well was transferred to a 96-well plate and 45 μL of ACN:0.1% NaOH (50:50, v/v)⁺0.20 nM d8-KMV was added and mixed on a shaker. 15 μL was transferred to a new 96-well plate and 15 μL of 10 mg/mL PFBHA solution was added and mixed on the shaker. The reaction was placed on ice for 30 minutes and quenched with 10 μL of ACN: Acetone (50:50, v/v) on a shaker. The resulting mixture was diluted with 100 μL of ACN:0.1% NaOH (50:50, v/v) on a shaker. 10 μL of sample were injected on the Shimadzu 20AD LC-MS/MS to quantify 13C6-KMV.

Specific compounds disclosed herein were tested in the foregoing assay and they were determined to inhibit BCAT2 in the cell with an IC₅₀ according to the following scores: (A) less than or equal to 0.100 μM, (B) greater than 0.100 and less than or equal to 0.500 μM, (C) greater than 0.500 and less than or equal to 1.000 μM, (d) greater than 1.000 μM, (E) no fit, and (NT) not tested, as shown in Table 6 below. Where a compound was tested multiple times, the average value of the tests is reported.

### BCAT2 In Vitro Enzyme Inhibition Assay

### Procedure:

2× Buffer: 200 mM Tris pH 8.0, 300 mM NaCl, 0.1% BSA

1.67× hBCAT2 Enzyme Mix: 0.33 μg/mL hBCAT2, 1× Buffer

2.5× Substrate Mix: 460 μM L-leucine, 420 μM α-KetoGlutarate, 10 U/mL glutamate dehydrogenase, 10 mM NAD, 30 μg/mL diaphorase, 20 μm Resazurin

BCAT2 enzyme stock solution was diluted to prepare a 1.67× hBCAT2 Enzyme Mix. 1 μL of test compound was first added to the reaction wells followed by 30 μL of 1.67× hBCAT2 Enzyme Mix and incubated at room temperature (25° C.) for one hour. Meanwhile the Substrate Mix was prepared. The reaction was initiated by the addition of 20 μL of the 2.5× Substrate Mix, bringing the final reaction mix of all components to 1×. Reactions were quenched after one hour by the addition of 25 μL of 6% SDS and reaction progress was measured via fluorescence read (Ex 540/Em 590).

### Test Compound Preparation:

Test compounds were prepared at 50× final concentration in DMSO, 1 to 3 dilutions were made for 10 points. The compounds were added to the assay as a 1 to 50 dilution (1 μL in 50 μL) to yield a top assay final concentration of 100 μM, decreasing 3-fold for 10 points. Each compound dilution series test also included an assay max (1 μL DMSO Added in lieu of compound, Full Reaction Components Included) and Min (1 μL DMSO Added in lieu of compound, 30 μl of 1.67× hBCAT2 Enzyme Mix replated by 30 μL of 1× Buffer). All tests were performed in technical duplicate.

Specific compounds disclosed herein were tested in the foregoing assay and they were determined to inhibit the BCAT2 enzyme with an IC₅₀ according to the following scores: (A) less than or equal to 0.100 μM, (B) greater than 0.100 and less than or equal to 0.500 μM, (C) greater than 0.500 and less than or equal to 1.000 μM, (d) greater than 1.000 μM, (E) no fit, and (NT) not tested, as shown in Table 6 below. Where a compound was tested multiple times, the average value of the tests is reported.

### BCAT1 In Vitro Enzyme Inhibition Assay

### Procedure:

2× Buffer: 200 mM Tris pH 8.0, 300 mM NaCl, 0.1% BSA

1.67× hBCAT1 Enzyme Mix: 0.125 μg/mL hBCAT1, 1× Buffer

2.5× Substrate Mix: 400 μM L-leucine, 130 μM α-KetoGlutarate, 10 U/mL glutamate dehydrogenase, 10 mM NAD, 30 μg/mL diaphorase, 20 μm Resazurin

BCAT1 enzyme stock solution was diluted to prepare a 1.67× hBCAT1 Enzyme Mix. 1 μL of test compound was first added to the reaction wells followed by 30 μL of 1.67× hBCAT1 Enzyme Mix and incubated at room temperature (25° C.) for one hour. Meanwhile the Substrate Mix was prepared. The reaction was initiated by the addition of 20 μL of the 2.5× Substrate Mix, bringing the final reaction mix of all components to 1×. Reactions were quenched after one hour by the addition of 25 μL of 6% SDS and reaction progress was measured via fluorescence read (Ex 540/Em 590).

### Test Compound Preparation:

Test compounds were prepared at 50× final concentration in DMSO, 1 to 3 dilutions were made for 10 points. The compounds were added to the assay as a 1 to 50 dilution (1 μL in 50 μL) to yield a top assay final concentration of 100 μM, decreasing 3-fold for 10 points. Each compound dilution series test also included an assay max (1 μL DMSO Added in lieu of compound, Full Reaction Components Included) and Min (1 μL DMSO Added in lieu of compound, 30 μl of 1.67× hBCAT1 Enzyme Mix replated by 30 μL of 1× Buffer). All tests were performed in technical duplicate.

Specific compounds disclosed herein were tested in the foregoing assay and they were determined to inhibit the BCAT1 enzyme with an IC₅₀ according to the following scores: (A) less than or equal to 0.500 μM, (B) greater than 0.500 and less than or equal to 1.000 μM, (C) greater than 1.000 and less than or equal to 5.000 μM, (D) greater than 5.000 μM, (E) no fit, and (NT) not tested, as shown in Table 6 below. Where a compound was tested multiple times, the average value of the tests is reported.

TABLE 6

| | BCAT2 Cell | BCAT2 Enzyme | BCAT1 Enzyme |
|---|---|---|---|
| Ex. No. | Inhibition | Inhibition | Inhibition |
| 1 | B | A | C |
| 2 | B | A | B |
| 3 | B | A | C |
| 4 | C | A | D |
| 5 | A | A | A |
| 6 | A | A | A |
| 7 | A | A | A |
| 8 | A | A | A |
| 9 | A | A | B |
| | B | | |
| 10 | C | A | C |
| 11 | A | A | A |
| 12 | B | A | A |
| 13 | A | A | A |
| 14 | A | A | A |
| 15 | A | A | A |
| 16 | A | A | A |
| 17 | NT | A | A |
| 18 | A | A | A |
| 19 | A | A | A |
| 20 | A | A | A |
| 21 | B | A | B |
| 22 | A | A | A |
| 23 | A | A | A |
| 24 | A | A | A |
| 25 | A | A | A |
| 26 | E | B | D |
| 27 | NT | A | C |
| 28 | C | A | C |
| 29 | B | A | B |
| 30 | C | A | C |
| 31 | A | A | A |
| 32 | D | B | C |
| 33 | C | A | C |
| 34 | E | B | D |
| 35 | C | A | C |
| 36 | B | A | B |
| 37 | D | B | C |
| 38 | B | A | C |
| 39 | A | A | A |
| 40 | NT | A | C |
| 41 | D | B | D |
| 42 | E | D | E |
| 43 | C | A | D |
| | B | | |
| 44 | E | D | D |
| 45 | E | D | D |
| 46 | E | D | D |
| 47 | E | C | D |
| 48 | E | D | D |
| 49 | E | B | D |
| 50 | E | A | A |
| 51 | NT | D | D |
| 52 | E | D | D |
| 53 | A | A | A |
| 54 | NT | A | D |
| 55 | A | A | A |
| 56 | B | A | C |
| 57 | NT | C | D |
| 58 | B | A | C |
| 59 | D | B | D |
| 60 | B | A | B |
| 61 | A | A | A |
| 62 | A | A | B |
| 63 | B | A | D |
| 64 | B | A | C |
| | C | | |
| 65 | A | A | B |
| 66 | A | A | A |
| 67 | D | B | D |
| 68 | A | A | A |
| 69 | D | B | D |
| 70 | NT | A | D |
| 71 | A | A | A |
| 72 | A | A | A |
| 73 | D | A | D |
| 74 | A | A | A |
| 75 | A | A | A |
| 76 | A | A | A |
| 77 | A | A | A |
| 78 | A | A | A |
| 79 | D | B | D |
| 80 | A | A | A |
| 81 | B | A | A |
| 82 | C | A | A |
| 83 | A | A | A |
| 84 | A | A | A |
| 85 | A | A | A |
| 86 | A | A | A |
| 87 | B | A | C |
| 88 | A | A | A |
| 89 | B | A | B |
| 90 | B | A | A |
| 91 | D | B | C |
| 92 | NT | D | D |
| 93 | NT | B | D |
| 94 | NT | D | D |
| 95 | E | D | NT |
| 96 | E | B | C |
| 97 | E | D | NT |
| 98 | D | B | D |
| 99 | E | A | C |
| 100 | E | C | NT |
| 101 | D | B | D |
| 102 | D | A | C |
| 103 | B | A | B |
| 104 | A | A | A |
| 105 | A | A | A |
| 106 | A | A | A |
| 107 | C | B | A |
| 108 | A | A | A |
| 109 | A | A | A |
| 110 | A | A | A |
| 111 | B | B | A |
| 112 | A | A | A |
| 113 | A | A | A |
| 114 | B | B | NT |
| 115 | A | A | A |
| 116 | A | A | A |
| 117 | A | A | B |
| 118 | A | A | A |
| 119 | B | A | A |
| 120 | D | A | C |
| | | | D |
| 121 | B | A | B |
| 122 | E | B | D |
| 123 | A | A | A |
| 124 | B | A | C |
| 125 | B | A | C |
| 126 | A | A | A |
| 127 | D | B | C |
| 128 | A | A | A |
| 129 | D | A | D |
| 130 | B | A | C |
| 131 | B | A | A |
| 132 | A | A | A |
| 133 | D | B | C |
| 134 | A | A | A |
| 135 | E | D | D |
| 136 | A | A | A |
| 137 | E | D | NT |
| 138 | B | A | B |
| 139 | E | D | D |
| 140 | E | D | NT |
| 141 | A | A | A |
| 142 | A | A | A |
| 143 | B | B | D |
| 144 | NT | C | D |
| 145 | NT | D | E |
| 146 | NT | D | D |
| 147 | NT | D | E |
| 148 | D | A | D |
| 149 | B | A | C |
| 150 | NT | C | D |
| 151 | NT | A | D |
| 152 | A | A | B |
| 153 | NT | B | D |
| 154 | NT | C | D |
| 155 | NT | A | C |
| 156 | NT | D | D |
| 157 | NT | A | C |
| 158 | D | A | C |
| 159 | B | A | A |
| 160 | C | A | B |
| 161 | B | A | C |
| 162 | A | A | A |
| 163 | A | A | A |
| 164 | B | B | D |
| | E | C | |
| 165 | A | A | A |
| 166 | B | A | A |
| 167 | NT | B | NT |
| 168 | A | A | A |
| 169 | A | A | A |
| 170 | A | A | A |
| 171 | D | A | A |
| 172 | C | A | A |
| 173 | C | A | C |
| 174 | NT | D | NT |
| 175 | A | A | A |
| 176 | D | B | C |
| 177 | E | C | NT |
| 178 | B | A | B |
| 179 | A | A | A |
| 180 | B | A | C |
| 181 | A | A | A |
| 182 | C | A | NT |
| 183 | B | A | A |
| 184 | A | A | A |
| 185 | A | A | A |
| 186 | A | A | A |
| 187 | B | B | C |
| 188 | B | A | C |
| 189 | A | A | A |
| 190 | E | D | NT |
| 191 | E | D | NT |
| 192 | D | A | C |
| 193 | E | D | NT |
| 194 | NT | C | NT |
| 195 | NT | B | C |
| 196 | A | A | A |
| 197 | NT | D | NT |
| 198 | D | D | NT |
| 199 | D | A | D |
| 200 | E | C | NT |
| 201 | E | C | NT |
| 202 | A | A | A |
| 203 | B | A | A |
| 204 | B | A | B |
| 205 | E | D | NT |
| 206 | NT | B | D |
| 207 | NT | B | D |
| 208 | E | D | NT |
| 209 | B | A | A |
| 210 | C | A | NT |
| 211 | B | A | A |
| 212 | NT | D | NT |
| 213 | C | A | B |
| 214 | A | A | A |
| 215 | E | C | NT |
| 216 | D | B | C |
| 217 | E | D | NT |
| 218 | A | A | A |
| 219 | D | B | NT |
| 220 | A | A | A |
| 221 | B | A | A |
| 222 | NT | D | NT |
| 223 | B | A | A |
| 224 | NT | D | NT |
| 225 | B | A | A |
| 226 | NT | D | NT |
| 227 | A | A | A |
| 228 | NT | D | NT |
| 229 | E | C | NT |
| 230 | D | A | B |
| 231 | E | D | NT |
| 232 | C | A | A |
| 233 | B | A | A |
| 234 | C | A | C |
| 235 | A | A | A |
| 236 | E | C | NT |
| 237 | C | B | NT |
| 238 | A | A | A |
| 239 | C | B | NT |
| 240 | A | A | A |
| 241 | B | B | NT |
| 242 | A | A | A |
| 243 | A | A | A |
| 244 | E | D | NT |
| 245 | A | A | A |
| 246 | A | A | A |
| 247 | C | C | NT |
| 248 | A | A | A |
| 249 | C | B | NT |
| 250 | A | A | A |
| 251 | D | B | NT |
| 252 | A | A | A |
| 253 | B | B | NT |
| 254 | A | A | A |
| 255 | B | A | NT |
| 256 | A | A | A |
| 257 | E | C | NT |
| 258 | A | A | A |
| 259 | D | B | NT |
| 260 | A | A | A |
| 261 | D | B | NT |
| 262 | D | B | D |
| 263 | E | D | NT |
| 264 | A | A | A |
| 265 | D | B | NT |
| 266 | E | B | NT |
| 267 | B | B | A |
| 268 | NT | C | D |
| 269 | D | D | NT |
| 270 | D | A | A |
| 271 | E | D | NT |
| 272 | C | A | B |
| 273 | E | C | NT |
| 274 | C | A | A |
| 275 | E | D | NT |
| 276 | A | A | A |
| 277 | E | D | NT |
| 278 | B | A | A |
| 279 | E | D | NT |
| 280 | C | A | C |
| 281 | A | A | A |
| 282 | NT | B | D |
| 283 | D | A | D |
| | | | C |
| 284 | NT | A | C |
| 285 | NT | A | NT |
| 286 | B | A | C |
| 287 | E | B | D |
| 288 | E | C | D |
| 289 | NT | B | D |
| 290 | NT | A | D |
| 291 | B | A | C |
| 292 | NT | A | C |
| 293 | NT | A | C |
| 294 | E | C | NT |
| | | D | |
| 300 | NT | D | D |
| 301 | NT | D | D |
| 302 | B | A | B |
| 303 | B | A | A |
| 304 | E | D | NT |
| 305 | B | A | A |
| 306 | B | A | B |
| 307 | E | D | NT |
| 308 | E | D | NT |
| 309 | E | D | NT |
| 310 | E | D | NT |
| 311 | C | A | C |
| 312 | A | A | A |
| 313 | A | A | A |
| 314 | B | A | A |
| 315 | B | A | B |
| 316 | D | A | C |
| 317 | A | A | A |
| 318 | E | C | NT |
| 319 | E | B | C |
| 320 | E | B | C |
| 321 | C | A | B |
| 322 | C | A | C |
| 323 | D | B | NT |
| 324 | B | A | C |
| 325 | A | A | A |
| 326 | A | A | A |
| 327 | NT | B | C |
| 328 | D | B | D |
| 329 | C | A | B |
| 330 | A | A | A |
| 331 | A | A | A |
| 332 | C | B | B |
| 333 | E | D | NT |
| 334 | C | A | A |
| 335 | E | B | NT |
| 336 | NT | B | C |
| 337 | C | A | A |
| 338 | A | A | A |
| 339 | A | A | A |
| 340 | A | A | A |
| 341 | A | A | C |
| 342 | A | A | A |
| 343 | B | A | C |
| 344 | A | A | A |
| 345 | C | B | B |
| 346 | D | B | D |
| 347 | A | A | A |
| 348 | A | A | A |
| 349 | B | A | A |
| 350 | B | A | B |
| 351 | C | B | C |
| 352 | A | A | A |
| 353 | D | D | NT |
| 354 | A | A | A |
| 355 | A | A | A |
| 356 | A | A | B |
| 357 | NT | C | NT |
| 358 | B | A | B |
| 359 | E | B | NT |
| 360 | NT | D | NT |
| 361 | NT | A | NT |
| 362 | NT | D | NT |
| 363 | A | A | A |
| 364 | A | A | A |
| 365 | D | A | C |
| 366 | D | A | B |
| 367 | E | A | A |
| 368 | NT | D | NT |
| 369 | E | C | NT |
| 370 | E | D | NT |
| 371 | E | D | NT |
| 372 | NT | A | A |
| 373 | D | A | NT |
| 374 | A | A | A |
| 375 | NT | C | NT |
| 376 | E | C | NT |
| 377 | D | C | NT |
| 378 | A | A | A |
| 379 | A | A | A |
| 380 | D | B | D |
| 381 | B | A | C |
| 382 | NT | A | NT |
| 383 | B | A | A |
| 384 | D | D | NT |
| 385 | B | A | A |
| 386 | A | A | A |
| 387 | A | A | A |
| 388 | E | D | NT |
| 389 | A | A | A |
| 390 | D | B | NT |
| 391 | E | D | NT |
| 392 | D | B | C |
| 393 | A | A | A |
| 394 | NT | D | C |
| 395 | A | A | A |
| 396 | D | A | NT |
| 397 | E | D | C |
| 398 | NT | A | A |
| 399 | A | A | A |
| 400 | A | A | A |
| 401 | A | A | A |
| 402 | A | A | A |
| 403 | B | A | A |
| 404 | C | B | NT |
| 405 | A | A | A |
| 406 | E | C | NT |
| 407 | A | A | A |
| 408 | NT | A | NT |
| 409 | B | A | B |
| 410 | NT | D | NT |
| 411 | D | B | NT |
| 412 | A | A | A |
| 413 | E | D | NT |
| 414 | A | A | A |
| 415 | E | D | NT |
| 416 | A | A | C |
| 417 | D | B | C |
| 418 | E | D | NT |
| 419 | A | A | NT |
| | B | | |
| 420 | A | A | A |
| 421 | B | A | B |
| 422 | NT | D | NT |
| 423 | NT | C | NT |
| 424 | A | A | A |
| 425 | NT | D | NT |
| 426 | B | A | B |
| 427 | B | A | B |
| 428 | E | D | NT |
| 429 | A | A | B |
| 430 | D | D | NT |
| 431 | D | C | NT |
| 432 | A | A | A |
| 433 | E | E | NT |
| 434 | E | E | NT |
| 435 | A | A | A |
| 436 | E | C | NT |
| 437 | A | A | A |
| 438 | NT | B | NT |
| 439 | A | A | A |
| 440 | E | C | NT |
| 441 | E | D | NT |
| 442 | B | A | C |
| 443 | E | D | NT |
| 444 | A | A | NT |
| 445 | NT | B | NT |
| 446 | A | A | NT |
| 447 | NT | B | NT |
| 448 | NT | C | NT |
| 449 | NT | A | NT |
| 450 | NT | D | NT |
| 451 | B | A | A |
| 452 | NT | D | NT |
| 453 | NT | B | NT |
| 454 | NT | A | A |
| 455 | B | A | A |
| 456 | D | A | A |
| 457 | NT | B | NT |
| 458 | A | A | A |
| 459 | B | A | NT |
| 460 | A | A | A |
| 461 | NT | A | NT |
| 462 | A | A | A |
| 463 | C | A | NT |
| 464 | NT | A | NT |
| 465 | A | A | NT |
| 466 | A | A | NT |

What is claimed:

1. A compound, wherein said compound is a compound of Formula IA or a compound of Formula IB: 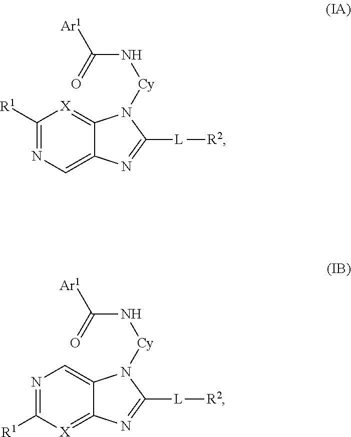
or a pharmaceutically acceptable salt thereof, wherein:
R¹ is optionally substituted heteroaryl, optionally substituted aryl, or optionally substituted 4-, 5- or 6-membered heterocycloalkyl;
X is CH or N;
Cy is optionally substituted C₅-C₇ cycloalkyl or optionally substituted 6- or 7-membered heterocycloalkyl;
L is a bond, —O—, —NR^{a}—, or optionally substituted C₁-C₄alkylene;
R^{a} is hydrogen or C₁-C₄alkyl;
R² is optionally substituted phenyl, optionally substituted pyridinyl, optionally substituted 5-membered heteroaryl, optionally substituted heterocycloalkyl, optionally substituted C₁-C₆alkyl, optionally substituted C₁-C₆alkoxy, optionally substituted C₁-C₆hydroxyalkyl, optionally substituted C₁-C₆haloalkyl, optionally substituted C₁-C₆haloalkoxy, optionally substituted C₃-C₆cycloalkyl, or NR^{f}R^{g};
R^{f} and R^{g} are each independently hydrogen, C₁-C₆alkyl, C₁-C₆haloalkyl, OH, C₁-C₄alkoxy, C₁-C₆hydroxyalkyl, or C₃-C₆cycloalkyl; and
Ar¹ is an optionally substituted five-membered heteroaryl.

2. The compound according to claim 1, or a pharmaceutically acceptable salt thereof, wherein the compound is a compound of Formula IA.

3. The compound according to claim 1, or a pharmaceutically acceptable salt thereof, wherein the compound is a compound of Formula IB.

4. The compound according to claim 2 or claim 3, or a pharmaceutically acceptable salt thereof, wherein X is CH.

5. The compound according to claim 2 or claim 3, or a pharmaceutically acceptable salt thereof, wherein X is N.

6. The compound according to any one of claims 1-5, or a pharmaceutically acceptable salt thereof, wherein Cy is optionally substituted C₅-C₇ cycloalkyl.

7. The compound according to claim 6, or a pharmaceutically acceptable salt thereof, wherein Cy is unsubstituted C₅-C₇ cycloalkyl.

8. The compound according to claim 6, or a pharmaceutically acceptable salt thereof, wherein Cy is C₅-C₇ cycloalkyl substituted with one or more of OH, deuterium, halo, cyano, C₁-C₃alkyl, C₁-C₃haloalkyl, C₁-C₃alkoxy, deuterated C₁-C₃alkoxy, C₁-C₃haloalkoxy, N₃, NH₂, NH(C₁-C₆alkyl), N(C₁-C₆alkyl)₂, OCO₂(C₁-C₄alkyl), OCO₂(C₃-C₄cycloalkyl), OSO₂(C₁-C₄alkyl), OSO₂(C₃-C₄cycloalkyl), OC(O)(C₁-C₄alkyl), OC(O)(C₃-C₄cycloalkyl), or NR^{m}Rⁿ; and
wherein R^{m} is hydrogen or C₁-C₄alkyl and Rⁿ is CO₂(C₁-C₄alkyl), C(O)C₁-C₄alkyl, SO₂(C₁-C₄alkyl), or SO₂(C₃-C₄cycloalkyl).

9. The compound according to claim 8, or a pharmaceutically acceptable salt thereof, wherein Cy is C₅-C₇ cycloalkyl substituted with one of OH, C₁-C₃alkoxy, cyano, OCO₂(C₁-C₄alkyl), OSO₂(C₁-C₄alkyl), N₃, or NH₂.

10. The compound according to claim 6, or a pharmaceutically acceptable salt thereof, wherein the optionally substituted C₅-C₇ cycloalkyl is an optionally substituted cyclohexyl or an optionally substituted cycloheptyl.

11. The compound according to claim 6, or a pharmaceutically acceptable salt thereof, wherein the optionally substituted C₅-C₇ cycloalkyl is an optionally substituted cyclopentyl.

12. The compound according to claim 11, or a pharmaceutically acceptable salt thereof, wherein the optionally substituted cyclopentyl is an unsubstituted cyclopentyl.

13. The compound according to claim 6, or a pharmaceutically acceptable salt thereof, wherein the optionally substituted C₅-C₇ cycloalkyl is an optionally substituted cyclohexyl.

14. The compound according to claim 13, or a pharmaceutically acceptable salt thereof, wherein the cyclohexyl is substituted with one or more of OH, deuterium, halo, cyano, C₁-C₃alkyl, C₁-C₃haloalkyl, C₁-C₃alkoxy, deuterated C₁-C₃alkoxy, C₁-C₃haloalkoxy, N₃, NH₂, NH(C₁-C₆alkyl), N(C₁-C₆alkyl)₂, OCO₂(C₁-C₄alkyl), OCO₂(C₃-C₄cycloalkyl), OSO₂(C₁-C₄alkyl), OSO₂(C₃-C₄cycloalkyl), OC(O)(C₁-C₄alkyl), OC(O)(C₃-C₄cycloalkyl), or NR^{m}Rⁿ; and
wherein R^{m} is hydrogen or C₁-C₄alkyl and Rⁿ is CO₂(C₁-C₄alkyl), C(O)C₁-C₄alkyl, SO₂(C₁-C₄alkyl), or SO₂(C₃-C₄cycloalkyl).

15. The compound according to claim 14, or a pharmaceutically acceptable salt thereof, wherein the cyclohexyl is substituted with one of OH, C₁-C₃alkoxy, cyano, OCO₂(C₁-C₄alkyl), OSO₂(C₁-C₄alkyl), N₃, or NH₂.

16. The compound according to claim 14, or a pharmaceutically acceptable salt thereof, wherein one carbon atom in the cyclohexyl ring is CF₂.

17. The compound according to claim 13, or a pharmaceutically acceptable salt thereof, wherein the optionally substituted cyclohexyl is an unsubstituted cyclohexyl.

18. The compound according to claim 6, or a pharmaceutically acceptable salt thereof, wherein the optionally substituted C₅-C₇ cycloalkyl is an optionally substituted cycloheptyl.

19. The compound according to claim 18, or a pharmaceutically acceptable salt thereof, wherein the optionally substituted cycloheptyl is an unsubstituted cycloheptyl.

20. The compound according to any one of claims 1-5, or a pharmaceutically acceptable salt thereof, wherein Cy is an optionally substituted 6-membered heterocycloalkyl.

21. The compound according to claim 20, or a pharmaceutically acceptable salt thereof, wherein Cy is an optionally substituted piperidinyl, an optionally substituted tetrahydropyranyl, an optionally substituted thianyl, an optionally substituted thianyl sulfone, an optionally substituted thianyl sulfoxide, or an optionally substituted 1-oxo-1-imino-1-thiacyclohexyl.

22. The compound according to any one of claims 20-21, or a pharmaceutically acceptable salt thereof, wherein Cy is an optionally substituted piperidinyl.

23. The compound according to claim 22, or a pharmaceutically acceptable salt thereof, wherein the nitrogen in the piperidinyl is substituted with C₁-C₆alkyl, C(O)C₁-C₆alkyl, C₁-C₆hydroxyalkyl, C(O)NR^{b}R^{c}, CO₂(C₁-C₆alkyl), SO₂(C₁-C₆alkyl), or SO₂(C₃-C₆cycloalkyl); and wherein each R^{b} and R^{c} is independently hydrogen or C₁-C₄alkyl.

24. The compound according to claim 22, or a pharmaceutically acceptable salt thereof, wherein the nitrogen in the piperidinyl is substituted with CH₃, C(O)CH₃, C(O)NH₂, CO₂CH₃, or SO₂CH₃.

25. The compound according to any one of claims 20-21, or a pharmaceutically acceptable salt thereof, wherein Cy is an optionally substituted tetrahydropyranyl.

26. The compound according to any one of claims 1-5, or a pharmaceutically acceptable salt thereof, wherein Cy is an optionally substituted 7-membered heterocycloalkyl.

27. The compound according to claim 26, or a pharmaceutically acceptable salt thereof, wherein the optionally substituted 7-membered heterocycloalkyl is an optionally substituted oxazepanyl, an optionally substituted thiepanyl, an optionally substituted thiepanyl-1-oxide, an optionally substituted thiepanyl-1,1-dioxide, an optionally substituted azepanyl, or an optionally substituted 1-imino-1-oxide-thiepanyl.

28. The compound according to any one of claims 1-27, or a pharmaceutically acceptable salt thereof, wherein R¹ is optionally substituted heteroaryl.

29. The compound according to claim 28, or a pharmaceutically acceptable salt thereof, wherein R¹ is optionally substituted 9-membered heteroaryl, such as optionally substituted benzofuranyl, optionally substituted isobenzofuranyl, optionally substituted benzoxazolyl, optionally substituted benzoisoxazolyl, optionally substituted benzothiazolyl, optionally substituted benzoisothiazolyl, optionally substituted indolyl, optionally substituted isoindolyl, optionally substituted indazolyl, optionally substituted benzimidazolyl, or optionally substituted benzothiophenyl.

30. The compound according to claim 28, or a pharmaceutically acceptable salt thereof, wherein R¹ is optionally substituted 5- or 6-membered heteroaryl.

31. The compound according to claim 30, or a pharmaceutically acceptable salt thereof, wherein R¹ is optionally substituted 5-membered heteroaryl.

32. The compound according to claim 31, or a pharmaceutically acceptable salt thereof, wherein the 5-membered heteroaryl is optionally substituted oxadiazolyl, optionally substituted thiadiazolyl, optionally substituted oxazolyl, optionally substituted pyrazolyl, optionally substituted imidazolyl, optionally substituted triazolyl, optionally substituted tetrazolyl, or optionally substituted thiazolyl.

33. The compound according to claim 32, or a pharmaceutically acceptable salt thereof, wherein the 5-membered heteroaryl is optionally substituted with one or more of oxo, NR^{b}R^{c}, C₁-C₄alkyl, C₁-C₄haloalkyl, C₁-C₆hydroxyalkyl, halo, cyano, C₁-C₄alkoxy, C₁-C₄haloalkoxy, or CO₂C₁-C₄alkyl; and wherein each R^{b} and R^{c} is independently hydrogen or C₁-C₄alkyl.

34. The compound according to claim 30, or a pharmaceutically acceptable salt thereof, wherein R¹ is optionally substituted 6-membered heteroaryl.

35. The compound according to claim 34, or a pharmaceutically acceptable salt thereof, wherein the optionally substituted 6-membered heteroaryl is optionally substituted pyridinyl, optionally substituted pyrimidinyl, or optionally substituted pyridazinyl.

36. The compound according to claim 35, or a pharmaceutically acceptable salt thereof, wherein the 6-membered heteroaryl is optionally substituted with one or more of oxo, NR^{b}R^{c}, C₁-C₄alkyl, C₁-C₄haloalkyl, C₁-C₄alkoxy, C₁-C₆hydroxyalkyl, cyano, halo, C₁-C₄haloalkoxy or CO₂(C₁-C₄alkyl); and
wherein each R^{b} and R^{c} is independently hydrogen or C₁-C₄alkyl.

37. The compound according to any one of claims 1-27, or a pharmaceutically acceptable salt thereof, wherein R¹ is optionally substituted aryl.

38. The compound according to claim 37, or a pharmaceutically acceptable salt thereof, wherein the optionally substituted aryl is phenyl, optionally substituted with one or more of C₁-C₆alkyl, C₁-C₆haloalkyl, C₁-C₆hydroxyalkyl, halo, cyano, or C₁-C₆cyanoalkyl.

39. The compound according to any one of claims 1-27, or a pharmaceutically acceptable salt thereof, wherein R¹ is optionally substituted 4-, 5- or 6-membered heterocycloalkyl.

40. The compound according to claim 39, or a pharmaceutically acceptable salt thereof, wherein R¹ is optionally substituted imidazolidinonyl, optionally substituted pyrrolidinyl, optionally substituted tetrahydrofuranyl, or optionally substituted azetidinyl.

41. The compound according to claim 40, or a pharmaceutically acceptable salt thereof, wherein R¹ is pyrrolidinyl, optionally substituted with one or more of oxo, C₁-C₆hydroxyalkyl, C₁-C₆alkyl, C₁-C₆alkoxy(alkylene), C₁-C₆cyanoalkyl, OH, cyano, CONR^{b}R^{c}, and wherein each R^{b} and R^{c} is independently hydrogen or C₁-C₄alkyl.

42. The compound according to any one of claims 1-41, or a pharmaceutically acceptable salt thereof, wherein L is a bond.

43. The compound according to any one of claims 1-41, or a pharmaceutically acceptable salt thereof, wherein L is —O—.

44. The compound according to any one of claims 1-41, or a pharmaceutically acceptable salt thereof, wherein L is —NR^{a}—, wherein R^{a} is hydrogen or C₁-C₄alkyl.

45. The compound according to any one of claims 1-41, or a pharmaceutically acceptable salt thereof, wherein L is optionally substituted C₁-C₄alkylene.

46. The compound according to claim 45, or a pharmaceutically acceptable salt thereof, wherein L is C₁-C₄alkylene substituted with deuterium, oxo, halo, NH₂, NH(C₁-C₄alkyl), N(C₁-C₄alkyl)₂, OH, C₁-C₃alkoxy, NR^{h}CO(C₁-C₄alkyl), NR^{h}CO₂(C₁-C₄alkyl), or an optionally substituted 4-, 5-, or 6-membered heterocycloalkyl containing a nitrogen wherein the nitrogen is connected to the alkylene carbon and each R^{h} is independently hydrogen or C₁-C₄alkyl.

47. The compound according to any one of claims 1-46, or a pharmaceutically acceptable salt thereof, wherein R² is optionally substituted phenyl.

48. The compound according to claim 47, or a pharmaceutically acceptable salt thereof, wherein the phenyl is optionally substituted with one or more of halo, C₁-C₄alkyl, C₃-C₄cycloalkyl, C₁-C₄alkoxy, C₁-C₄haloalkoxy, cyano, OH, NR^{b}R^{c}, C(O)NR^{b}R^{c}, CO₂(C₁-C₄alkyl), COOH, C₁-C₄alkylene-R²⁹, or O—C₁-C₄alkylene-R³⁰;
R²⁹ is cyano, NR^{b}R^{c}, or COOH;
R³⁰ is OH, OCH₃, or NR^{b}R^{c}; and
each R^{b} and each R^{c} is independently hydrogen or C₁-C₄alkyl.

49. The compound according to claim 47, or a pharmaceutically acceptable salt thereof, wherein R² is

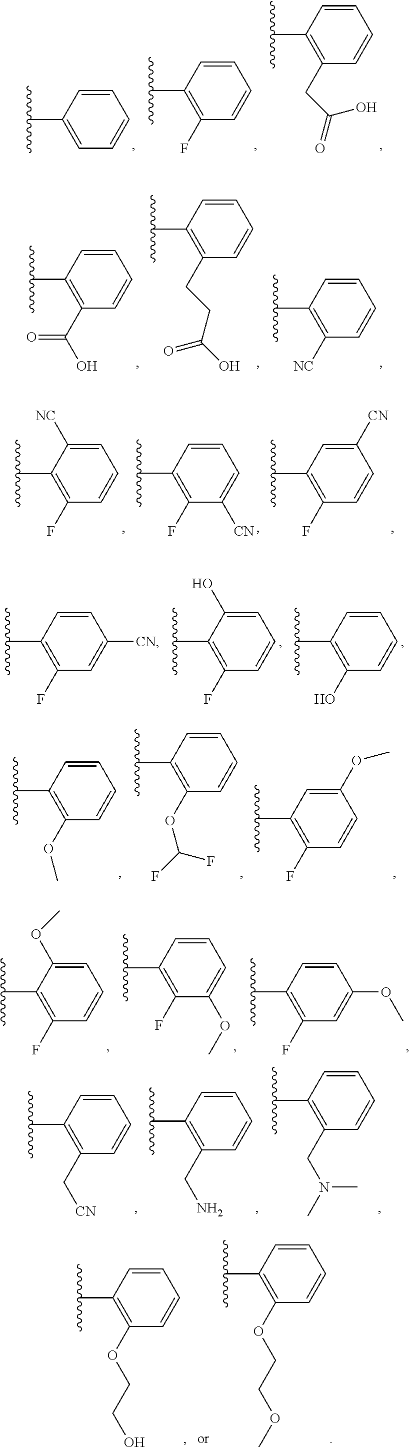

50. The compound according to any one of claims 1-46, or a pharmaceutically acceptable salt thereof, wherein R² is optionally substituted pyridinyl.

51. The compound according to claim 50, or a pharmaceutically acceptable salt thereof, wherein the pyridinyl is optionally substituted with one or more halo or C₁-C₄alkyl.

52. The compound according to any one of claims 1-46, or a pharmaceutically acceptable salt thereof, wherein R² is optionally substituted 5-membered heteroaryl.

53. The compound according to claim 52, or a pharmaceutically acceptable salt thereof, wherein the optionally substituted 5-membered heteroaryl is optionally substituted triazolyl, optionally substituted pyrazolyl, optionally substituted oxazolyl, or optionally substituted isoxazolyl.

54. The compound according to claim 53, or a pharmaceutically acceptable salt thereof, wherein R² is

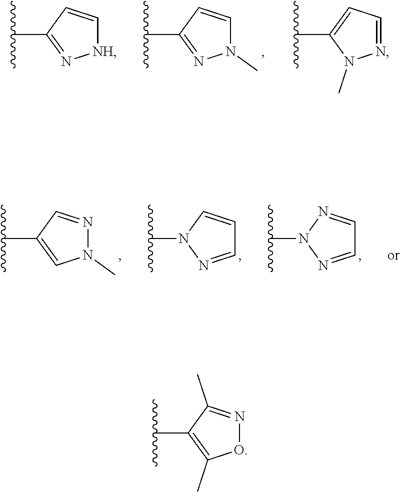

55. The compound according to any one of claims 1-46, or a pharmaceutically acceptable salt thereof, wherein R² is optionally substituted 4-, 5- or 6-membered heterocycloalkyl.

56. The compound according to claim 55, or a pharmaceutically acceptable salt thereof, wherein the optionally substituted heterocycloalkyl is optionally substituted azetidinyl, optionally substituted pyrrolidinyl, optionally substituted tetrahydropyranyl, optionally substituted piperidinyl, optionally substituted morpholinyl, or optionally substituted dioxanyl.

57. The compound according to claim 56, or a pharmaceutically acceptable salt thereof, wherein R² is

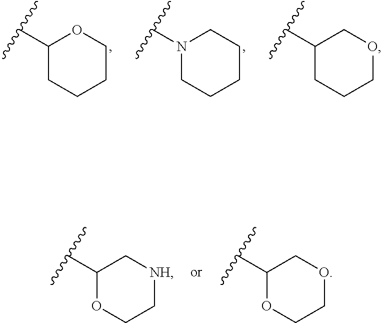 [SMILES: *C1CCCCO1 ; *N1CCCCC1 ; *C1CCCOC1 ; *C1CNCCO1 ; *C1COCCO1]

58. The compound according to any one of claims 1-46, or a pharmaceutically acceptable salt thereof, wherein R² is optionally substituted C₁-C₆alkyl.

59. The compound according to claim 58, or a pharmaceutically acceptable salt thereof, wherein the C₁-C₆alkyl is CH₃, CH(CH₃)₂, CH₂CH(CH₃)₂, C(CH₃)₃, or CH₂C(CH₃)₃.

60. The compound according to any one of claims 1-46, or a pharmaceutically acceptable salt thereof, wherein R² is optionally substituted C₁-C₆alkoxy.

61. The compound according to claim 60, or a pharmaceutically acceptable salt thereof, wherein the C₁-C₆alkoxy is OCH₃, or OCH(CH₃)₂.

62. The compound according to any one of claims 1-46, or a pharmaceutically acceptable salt thereof, wherein R² is optionally substituted C₁-C₆hydroxyalkyl.

63. The compound according to claim 62, or a pharmaceutically acceptable salt thereof, wherein the C₁-C₆hydroxyalkyl is C(OH)(CH₃)₂ or CH(OH)(CH₃).

64. The compound according to any one of claims 1-46, or a pharmaceutically acceptable salt thereof, wherein R² is optionally substituted C₁-C₆haloalkyl.

65. The compound according to claim 64, or a pharmaceutically acceptable salt thereof, wherein the C₁-C₆haloalkyl is CF₃, CH(CH₃)(CF₃), CH₂CF₃, C(CH₃)₂F, CH₂C(CH₃)₂F, C(CH₃)F₂, CH₂C(CH₃)F₂, CHFCH(CH₃)₂, or CF₂CH(CH₃)₂.

66. The compound according to any one of claims 1-46, or a pharmaceutically acceptable salt thereof, wherein R² is optionally substituted C₁-C₆haloalkoxy.

67. The compound according to claim 66, or a pharmaceutically acceptable salt thereof, wherein the C₁-C₆haloalkoxy is OCF₃, OCH₂CF₃, OCHF₂, OCH₂F, or OCH(CH₃)(CF₃).

68. The compound according to any one of claims 1-46, or a pharmaceutically acceptable salt thereof, wherein R² is optionally substituted C₃-C₆cycloalkyl.

69. The compound according to claim 68, or a pharmaceutically acceptable salt thereof, wherein the C₃-C₆cycloalkyl is optionally substituted with one or more of C₁-C₄alkyl or C₁-C₄haloalkyl.

70. The compound according to any of claims 1-46, or a pharmaceutically acceptable salt thereof, wherein R² is NR⁹.

71. The compound according to any one of the claims 1-70, or a pharmaceutically acceptable salt thereof, wherein Ar¹ is an optionally substituted thiophenyl or an optionally substituted thiazolyl.

72. The compound according to any one of claims 1-2, 4-6, 10, 13, or 285-71, wherein the compound of Formula IA is a compound of Formula IA-1: 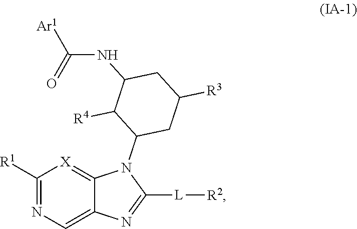
or a pharmaceutically acceptable salt thereof, wherein R³ is hydrogen, OH, C₁-C₃alkoxy, cyano, OCO₂(C₁-C₃alkyl), OSO₂(C₁-C₃alkyl), N₃, NH₂, NH(C₁-C₆alkyl), or N(C₁-C₆alkyl)₂; R⁴ is hydrogen, OH, C₁-C₃alkoxy, cyano, OCO₂(C₁-C₃alkyl), OSO₂(C₁-C₃alkyl), N₃, NH₂, NH(C₁-C₆alkyl), or N(C₁-C₆alkyl)₂, and wherein at least one of R³ and R⁴ is hydrogen.

73. The compound according to claim 72, wherein the compound of Formula IA-1 is a compound of Formula IA-1a: 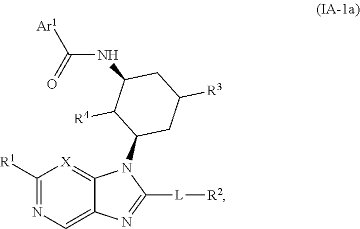
or a pharmaceutically acceptable salt thereof.

74. The compound according to claim 72 or claim 73, or a pharmaceutically acceptable salt wherein R³ is OH, C₁-C₃alkoxy, cyano, OSO₂(C₁-C₃alkyl), OSO₂(C₁-C₃alkyl), N₃, NH₂, NH(C₁-C₆alkyl), or N(C₁-C₆alkyl)₂; and R⁴ is hydrogen.

75. The compound according to claim 72 or claim 73, or a pharmaceutically acceptable salt wherein R³ is hydrogen and R⁴ is OH, C₁-C₃alkoxy, cyano, OCO₂(C₁-C₃alkyl), OSO₂(C₁-C₃alkyl), N₃, NH₂, NH(C₁-C₆alkyl), or N(C₁-C₆alkyl)₂.

76. The compound according to claim 72 or 73, or a pharmaceutically acceptable salt thereof, wherein R³ is OH or C₁-C₃alkoxy and R⁴ is hydrogen.

77. The compound according to claim 72 or 73, or a pharmaceutically acceptable salt thereof, wherein R³ is hydrogen and R⁴ is OH or C₁-C₃alkoxy.

78. The compound according to any one of claims 72-77, or a pharmaceutically acceptable salt thereof, wherein the C₁-C₃alkoxy is OCH₃.

79. The compound according to any one of claims 1-2, 4-6, 10, 18, or 28-71, wherein the compound of Formula IA is a compound of Formula IA-2 or Formula IA-3: 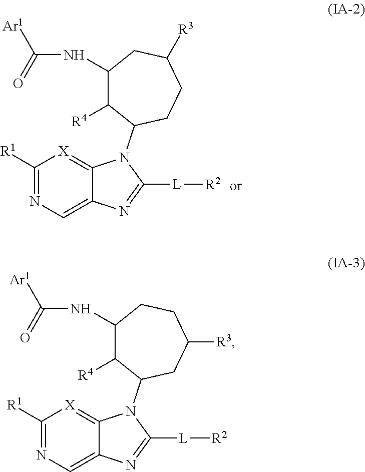
or a pharmaceutically acceptable salt thereof, wherein R³ is hydrogen, OH, or C₁-C₃alkoxy; R⁴ is hydrogen, OH, or C₁-C₃alkoxy, and wherein at least one of R³ and R⁴ is hydrogen.

80. The compound according to claim 79, wherein the compound of Formula IA-2 is a compound of Formula IA-2a, or a pharmaceutically acceptable salt thereof, and the compound of Formula IA-3 is a compound of Formula IA-3a, or a pharmaceutically acceptable salt thereof:

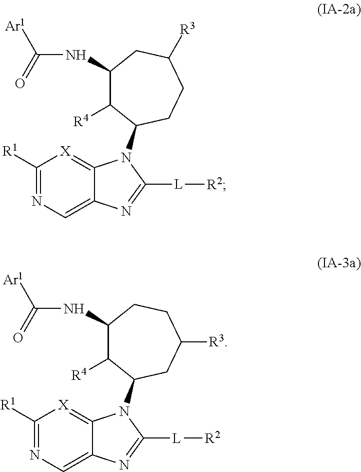

81. The compound according to claim 79 or 80, or a pharmaceutically acceptable salt thereof, wherein R³ is hydrogen and R⁴ is OH or C₁-C₃alkoxy.

82. The compound according to any one of claims 1-2, 4-5, 20, and 28-71, wherein the compound of Formula IA is a compound of Formula IA-4: 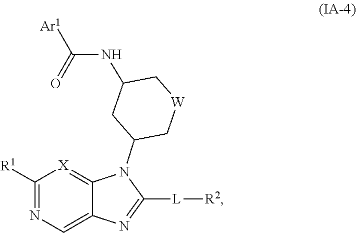
or a pharmaceutically acceptable salt thereof, wherein W is N—R⁵, S, SO, SO₂, or O;
R⁵ is hydrogen, C₁-C₆alkyl, C(O)C₁-C₆alkyl, oxo, OH, C₁-C₆alkoxy, C(O)NR^{b}R^{c}, C₁-C₆hydroxyalkyl, CO₂(C₁-C₆alkyl), SO₂(C₁-C₆alkyl), or SO₂(C₃-C₆cycloalkyl); and
each R^{b} and R^{c} is independently hydrogen or C₁-C₄alkyl.

83. The compound according to claim 82, wherein the compound of Formula IA is a compound of Formula IA-4a: 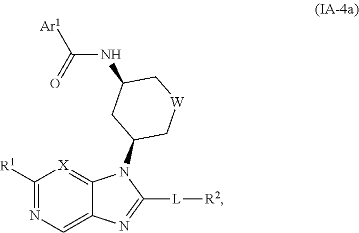
or a pharmaceutically acceptable salt thereof.

84. The compound according to claim 82 or claim 83, or a pharmaceutically acceptable salt thereof, wherein W is N—R⁵ or SO₂.

85. The compound according to claim 82 or claim 83, or a pharmaceutically acceptable salt thereof, wherein W is N—R⁵.

86. The compound according to claim 85, or a pharmaceutically acceptable salt thereof, wherein R⁵ is CH₃, COCH₃, CO₂CH₃, or SO₂CH₃.

87. The compound according to claim 82 or claim 83, or a pharmaceutically acceptable salt thereof, wherein W is O.

88. The compound according to any one of claims 1-2, and 4-71, wherein the compound of Formula IA is a compound of Formula IA-5: 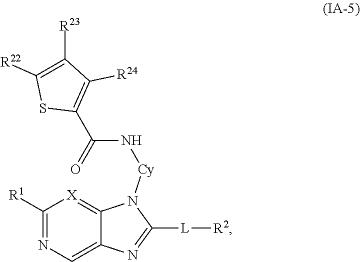
or a pharmaceutically acceptable salt thereof, wherein R²² is halo or optionally substituted C₂-C₆alkynyl; and R²³ and R²⁴ are hydrogen.

89. The compound according to any one of claims 1-2, and 4-71, wherein the compound of Formula IA is a compound of Formula IA-6: 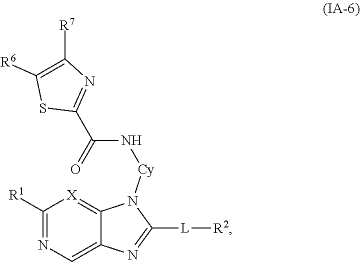
or a pharmaceutically acceptable salt thereof, wherein R⁶ is halo, C₁-C₆haloalkyl, cyano, or optionally substituted C₂-C₆alkynyl; and R⁷ is hydrogen.

90. The compound according to any one of claims 1-2, 4-42, 47, and 71, wherein the compound of Formula IA is a compound of Formula IA-7: 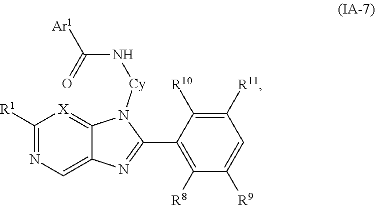
or a pharmaceutically acceptable salt thereof, wherein R⁸, R⁹, R¹⁰, and R¹¹ are each independently hydrogen, halo, C₁-C₄alkyl, C₃-C₄cycloalkyl, C₁-C₄alkoxy, C₁-C₄haloalkoxy, cyano, OH, NR^{b}R^{c}, C(O)NR^{b}R^{c}, CO₂(C₁-C₄alkyl), COOH, C₁-C₄alkylene-R²⁹, or O—C₁-C₄alkylene-R³⁰;
R²⁹ is cyano, NR^{b}R^{c}, or COOH;
R³⁰ is OH, OCH₃, or NR^{b}R^{c}; and
each R^{b} and each R^{C} is independently hydrogen or C₁-C₄alkyl.

91. The compound according to claim 90, or a pharmaceutically acceptable salt thereof, wherein at least two of R⁸, R⁹, R¹⁰, and R¹¹ are hydrogen.

92. The compound according to claim 90, or a pharmaceutically acceptable salt thereof, wherein at least three of R⁸, R⁹, R¹⁰, and R¹¹ are hydrogen.

93. The compound according to any one of claims 90-92, or a pharmaceutically acceptable salt thereof, wherein at least one of R⁸, R⁹, R¹⁰, and R¹¹ is F, CH₃, OCH₃, OH, or cyano.

94. The compound according to any one of claims 1-2, 4-42, 50, and 71, wherein the compound of Formula IA is a compound of Formula IA-8: 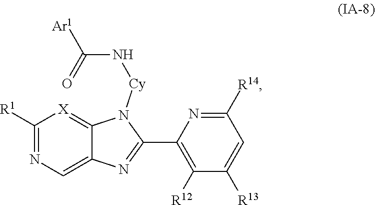
or a pharmaceutically acceptable salt thereof, wherein R¹², R¹³, and R¹⁴ are each independently hydrogen, halo, C₁-C₄alkyl, C₃-C₄cycloalkyl, C₁-C₄alkoxy, C₁-C₄haloalkoxy, cyano, OH, NR^{b}R^{c}, C(O)NR^{b}R^{c}, CO₂C₁-C₄alkyl, COOH, C₁₋₄alkylene-R²⁹, or O—C₁-C₄alkylene-R³⁰;
R²⁹ is cyano, NR^{b}R^{c}, or COOH;
R³⁰ is OH OCH₃, or NR^{b}R^{c}; and
each R^{b} and each R^{c} is independently hydrogen or C₁-C₄alkyl.

95. The compound according to claim 94, or a pharmaceutically acceptable salt thereof, wherein at least two of R¹², R¹³, and R¹⁴ are hydrogen.

96. The compound according to claim 94 or claim 95, or a pharmaceutically acceptable salt thereof, wherein one of R¹², R¹³, and R¹⁴ is F or CH₃.

97. The compound according to any one of claims 1-2, 4-28, 30-32, and 42-71, wherein the compound of Formula IA is a compound of Formula IA-9: 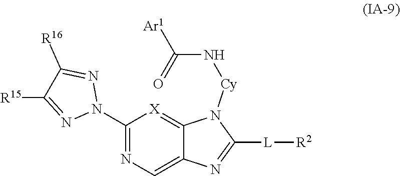
or a pharmaceutically acceptable salt thereof, wherein R¹⁵ and R¹⁶ are each independently hydrogen, halo, C₁-C₆alkyl, C₁-C₆haloalkyl, C₁-C₆hydroxyalkyl, CO₂(C₁-C₆alkyl), or CONR^{b}R^{c}; and
each R^{b} and each R^{c} is independently hydrogen or C₁-C₄alkyl.

98. The compound according to claim 97, or a pharmaceutically acceptable salt thereof, wherein one of R¹⁵ or R¹⁶ is hydrogen and the other of R¹⁵ or R¹⁶ is hydrogen, CH₃, CO₂CH₃, CH₂OH, CH(OH)(CH₃), or C(CH₃)₂OH.

99. The compound according to any one of claims 1-2, 4-28, 30-32, and 42-71, wherein the compound of Formula IA is a compound of Formula IA-10: 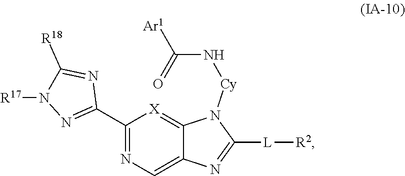
or a pharmaceutically acceptable salt thereof, wherein R¹⁷ is hydrogen, C₁-C₆alkyl, or C₁-C₆haloalkyl;
R¹⁸ is hydrogen, halo, C₁-C₆alkyl, CO₂(C₁-C₆alkyl), or CONR^{b}R^{c}; and
each R^{b} and each R^{c} is independently hydrogen or C₁-C₄alkyl.

100. The compound according to any one of claims 1-2, 4-28, 30-32, and 42-71, wherein the compound of Formula IA is a compound of Formula IA-11: 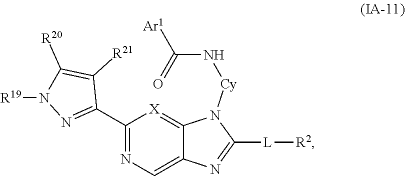
or a pharmaceutically acceptable salt thereof, wherein R¹⁹ is hydrogen, C₁-C₆alkyl, or C₁-C₆haloalkyl;
and R²⁰ and R²¹ are each independently hydrogen, halo, C₁-C₆alkyl, C₁-C₆haloalkyl, CO₂(C₁-C₆alkyl), or CONR^{b}R^{c}; and
each R^{b} and each R^{c} is independently hydrogen or C₁-C₄alkyl.

101. The compound according to any one of claims 1-2, 4-28, 30-32, or 42-71, wherein the compound of Formula IA is a compound of Formula IA-12: 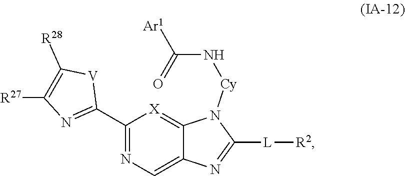
or a pharmaceutically acceptable salt thereof, wherein:
V is O or S;
R²⁷ is hydrogen, C₁-C₆alkyl, C₁-C₆haloalkyl, halo, C₁-C₆hydroxyalkyl, or NR^{b}R^{c};
R²⁸ is hydrogen or C₁-C₆alkyl; and
each R^{b} and R^{c} is independently hydrogen or C₁-C₄alkyl.

102. The compound according to any one of claims 1-2, 4-28, 30, 34, 35, or 42-71, wherein the compound of Formula IA is a compound of Formula IA-13: 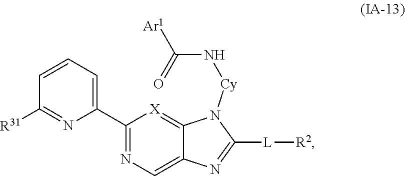
or a pharmaceutically acceptable salt thereof, wherein:
R³¹ is hydrogen, C₁-C₆alkyl, C₁-C₆haloalkyl, halo, C₁-C₆alkoxy, C₁-C₆hydroxyalkyl, cyano, or NR^{b}R^{c}; and
each R^{b} and R^{c} is independently hydrogen or C₁-C₄alkyl.

103. The compound according to any one of claims 1, 3-7, 10, 13, 17 or 28-71,
wherein the compound of Formula IB is a compound of Formula IB-1: 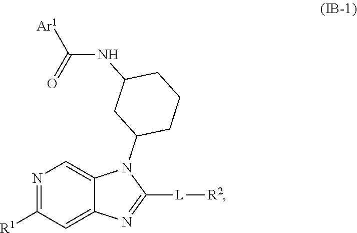
or a pharmaceutically acceptable salt thereof.

104. The compound according to claim 103, wherein the compound of Formula IB-1 is a compound of Formula IB-1a: 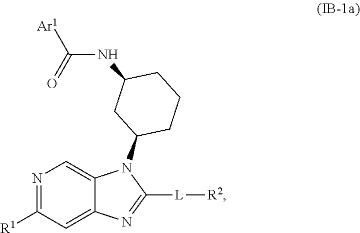
or a pharmaceutically acceptable salt thereof.

105. The compound according to claim 103 or claim 104, or a pharmaceutically acceptable salt thereof, wherein R¹ is optionally substituted triazolyl, optionally substituted oxadiazolyl, optionally substituted pyrazolyl, or optionally substituted oxazolyl.

106. The compound according to any one of claims 103-105, or a pharmaceutically acceptable salt thereof, wherein L is a bond and R² is optionally substituted phenyl or optionally substituted pyridinyl.

107. The compound according to any one of claims 103-106, or a pharmaceutically acceptable salt thereof, wherein Ar¹ is optionally substituted thiophenyl or optionally substituted thiazolyl.

108. A compound, wherein said compound is a compound of Formula IIA or Formula IIB: 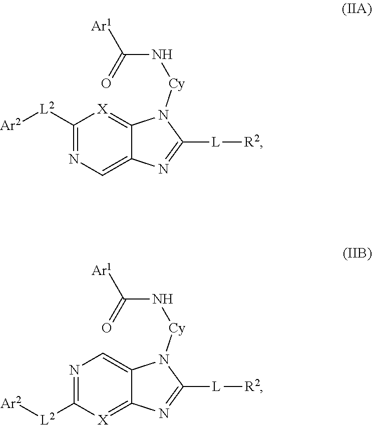
or a pharmaceutically acceptable salt thereof, wherein:
X is CH or N;
Cy is optionally substituted C₅-C₇ cycloalkyl or optionally substituted 6- or 7-membered heterocycloalkyl;
L is a bond, —O—, —NR^{a}—, or optionally substituted C₁-C₄alkylene;
L² is —O—, —NR^{a}—, or —CH₂—;
each R^{a} is independently hydrogen or C₁-C₄alkyl;
R² is optionally substituted phenyl, optionally substituted pyridinyl, optionally substituted 5-membered heteroaryl, optionally substituted heterocycloalkyl, optionally substituted C₁-C₆alkyl, optionally substituted C₁-C₆alkoxy, optionally substituted C₁-C₆hydroxyalkyl, optionally substituted C₁-C₆haloalkyl, optionally substituted C₁-C₆haloalkoxy, optionally substituted C₃-C₆cycloalkyl, or NR^{f}R^{g};
R^{f} and R^{g} are each independently hydrogen, C₁-C₆alkyl, C₁-C₆haloalkyl, OH, C₁-C₄alkoxy, C₁-C₆hydroxyalkyl, or C₃-C₆cycloalkyl;
Ar¹ is an optionally substituted five-membered heteroaryl; and
Ar² is optionally substituted heteroaryl, optionally substituted aryl, or optionally substituted 4-, 5- or 6-membered heterocycloalkyl.

109. The compound according to claim 108, or a pharmaceutically acceptable salt thereof, wherein X is CH.

110. The compound according to claim 108 or claim 109, or a pharmaceutically acceptable salt thereof, wherein L² is —O—.

111. The compound according to any one of claims 108-110, or a pharmaceutically acceptable salt thereof, wherein Ar² is optionally substituted 5- or 6-membered heteroaryl or optionally substituted 4-, 5- or 6-membered heterocycloalkyl.

112. The compound according to claim 111, or a pharmaceutically acceptable salt thereof, where Ar² is optionally substituted tetrahydrofuranyl or optionally substituted pyrazolyl.

113. The compound according to any one of claims 108-112, or a pharmaceutically acceptable salt thereof, wherein L is a bond and R² is optionally substituted phenyl or optionally substituted pyridinyl.

114. The compound according to any one of claims 108-113, or a pharmaceutically acceptable salt thereof, wherein Ar¹ is optionally substituted thiophenyl or optionally substituted thiazolyl.

115. A compound, wherein said compound is a compound of Formula IIIA, Formula IIIB, Formula IIIC, or Formula IIID: 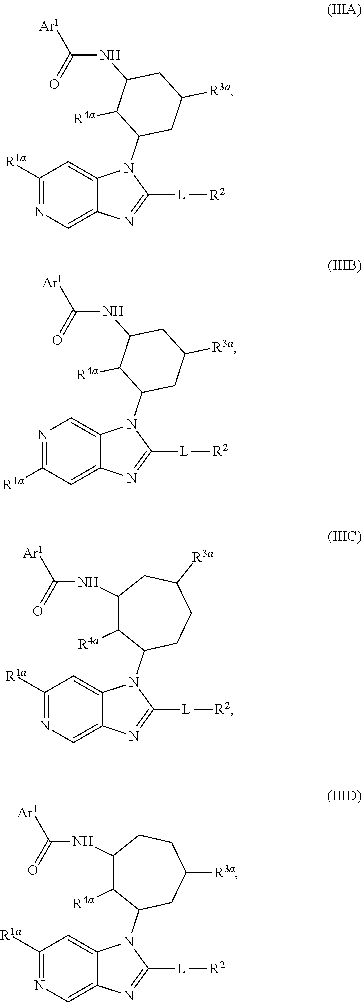
or a pharmaceutically acceptable salt thereof, wherein:
L is a bond, —O—, —NR^{a}—, or optionally substituted C₁-C₄alkylene;
R^{a} is hydrogen or C₁-C₄alkyl;
R^{1a} is hydrogen, C₁-C₆alkyl, C₁-C₆alkoxy, halo, C₁-C₆haloalkyl, C₁-C₆ haloalkoxy, CONR^{j}(C₁-C₄alkyl), CONR^{j}(C₃-C₄cycloalkyl), or NR^{b}R^{c};
R^{b} and R^{c} are each independently hydrogen or C₁-C₄alkyl;
each R^{j} is independently hydrogen or C₁-C₄alkyl;
R² is optionally substituted phenyl, optionally substituted pyridinyl, optionally substituted 5-membered heteroaryl, optionally substituted heterocycloalkyl, optionally substituted C₁-C₆alkyl, optionally substituted C₁-C₆alkoxy, optionally substituted C₁-C₆hydroxyalkyl, optionally substituted C₁-C₆haloalkyl, optionally substituted C₁-C₆haloalkoxy, optionally substituted C₃-C₆cycloalkyl, or NR^{f}R^{g};
R^{f} and R^{g} are each independently hydrogen, C₁-C₆alkyl, C₁-C₆haloalkyl, OH, C₁-C₄alkoxy, C₁-C₆hydroxyalkyl, or C₃-C₆cycloalkyl;
one of R^{3a} or R^{4a} is hydrogen, deuterium, OH, cyano, C₁-C₃alkyl, C₁-C₃haloalkyl, C₁-C₃alkoxy, C₁-C₃haloalkoxy, deuterated C₁-C₃alkoxy, NH₂, NH(C₁-C₄alkyl), N(C₁-C₄alkyl)₂, OCO₂(C₁-C₄alkyl), OSO₂(C₃-C₄cycloalkyl), OSO₂(C₁-C₄alkyl), OSO₂(C₃-C₄cycloalkyl), OC(O)(C₁-C₄alkyl), OC(O)(C₃-C₄cycloalkyl), or NR^{m}Rⁿ; and
the other of R^{3a} or R^{4a} is OH, cyano, C₁-C₃alkyl, C₁-C₃haloalkyl, C₁-C₃alkoxy, C₁-C₃haloalkoxy, deuterated C₁-C₃alkoxy, NH₂, NH(C₁-C₄alkyl), N(C₁-C₄alkyl)₂, OCO₂(C₁-C₄alkyl), OCO₂(C₃-C₄cycloalkyl), OSO₂(C₁-C₄alkyl), OSO₂(C₃-C₄cycloalkyl), OC(O)(C₁-C₄alkyl), OC(O)(C₃-C₄cycloalkyl), or NR^{m}Rⁿ; and
wherein each R^{m} is independently hydrogen or C₁-C₄alkyl and each Rⁿ is independently CO₂(C₁-C₄alkyl), C(O)C₁-C₄alkyl, SO₂C₁-C₄alkyl, or SO₂(C₃-C₄cycloalkyl); and
Ar¹ is an optionally substituted five-membered heteroaryl.

116. The compound according to claim 115, or a pharmaceutically acceptable salt thereof, wherein the compound is a compound of Formula IIIA or Formula IIIB.

117. The compound according to claim 116, wherein the compound of Formula IIIA is a compound of Formula IIIA-1, or a pharmaceutically acceptable salt thereof, and the compound of Formula IIIB is a compound of Formula IIIB-1, or a pharmaceutically acceptable salt thereof:

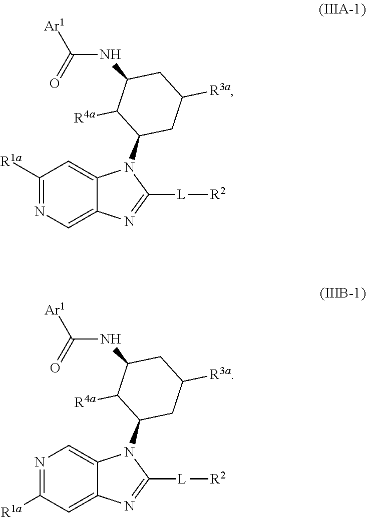

118. The compound according to claim 115, or a pharmaceutically acceptable salt thereof, wherein the compound is a compound of Formula IIIC or Formula IIID.

119. The compound according to claim 118, wherein the compound of Formula IIIC is a compound of Formula IIIC-1, or a pharmaceutically acceptable salt thereof, and the compound of Formula IIID is a compound of Formula IIID-1, or a pharmaceutically acceptable salt thereof:

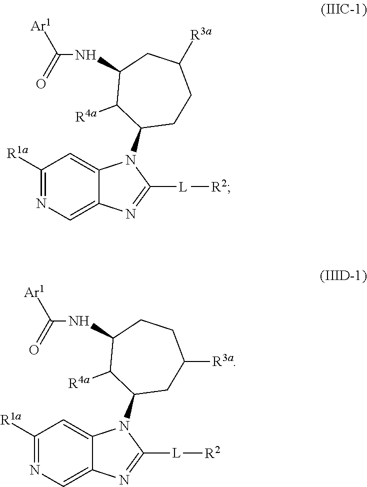

120. The compound according to any one of claims 115-119, or a pharmaceutically acceptable salt thereof, wherein one of R^{3a} or R^{4a} is OH or C₁-C₃alkoxy; and
the other of R^{3a} or R^{4a} is hydrogen.

121. The compound according to any one of claims 115-120, or a pharmaceutically acceptable salt thereof, wherein L is a bond and R² is optionally substituted phenyl or optionally substituted pyridinyl.

122. The compound according to any one of claims 115-121, or a pharmaceutically acceptable salt thereof, wherein Ar¹ is optionally substituted thiophenyl or optionally substituted thiazolyl.

123. The compound according to any one of claims 115-122, or a pharmaceutically acceptable salt thereof, wherein R^{1a} is hydrogen, NH₂, NHCH₃, F, OCH₃, or C(O)NH(CH₃).

124. A compound, wherein said compound is a compound of Formula IVA or a compound of Formula IVB: 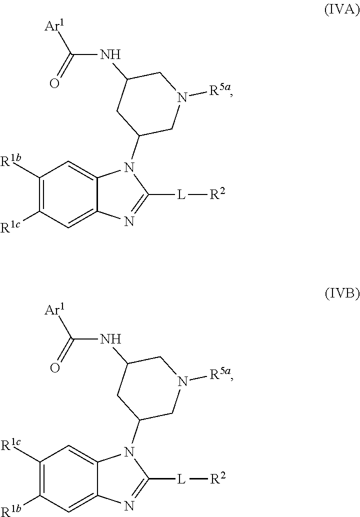
or a pharmaceutically acceptable salt thereof, wherein:
L is a bond, —O—, —NR^{a}—, or optionally substituted C₁-C₄alkylene;
R^{a} is hydrogen or C₁-C₄alkyl;
R^{1b} is optionally substituted heteroaryl, optionally substituted aryl, optionally substituted 4-, 5- or 6-membered heterocycloalkyl, CONR^{j}(C₁-C₄alkyl), CONR^{j}(C₃-C₄cycloalkyl), or cyano;
each R^{j} is independently hydrogen or C₁-C₄alkyl;
R^{1c} is hydrogen, halo, C₁-C₆alkyl, C₁-C₆haloalkyl, C₁-C₆alkoxy, or C₁-C₆haloalkoxy;
R² is optionally substituted phenyl, optionally substituted pyridinyl, optionally substituted 5-membered heteroaryl, optionally substituted heterocycloalkyl, optionally substituted C₁-C₆alkyl, optionally substituted C₁-C₆alkoxy, optionally substituted C₁-C₆hydroxyalkyl, optionally substituted C₁-C₆haloalkyl, optionally substituted C₁-C₆haloalkoxy, optionally substituted C₃-C₆cycloalkyl, or NR^{f}R^{g};
R^{f} and R^{g} are each independently hydrogen, C₁-C₆alkyl, C₁-C₆haloalkyl, OH, C₁-C₄alkoxy, C₁-C₆hydroxyalkyl, or C₃-C₆cycloalkyl;
R^{5a} is hydrogen, C₁-C₆alkyl, C(O)C₁-C₆alkyl, C(O)NR^{b}R^{c}, oxo, OH, C₁-C₆alkoxy, C₁-C₆hydroxyalkyl, CO₂(C₁-C₆alkyl), SO₂(C₁-C₆alkyl), or SO₂(C₃-C₆cycloalkyl);
each R^{b} and each R^{c} is independently hydrogen or C₁-C₄alkyl; and
Ar¹ is an optionally substituted five-membered heteroaryl.

125. The compound according to claim 124, wherein the compound of Formula IVA is a compound of Formula IVA-1, or a pharmaceutically acceptable salt thereof, and the compound of Formula IVB is a compound of Formula IVB-1, or a pharmaceutically acceptable salt thereof:

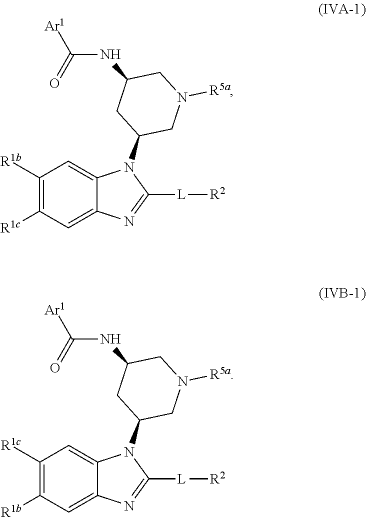

126. The compound according to claim 124 or claim 125, or a pharmaceutically acceptable salt thereof, wherein L is a bond and R² is optionally substituted phenyl or optionally substituted pyridinyl.

127. The compound according to any one of claims 124-126, or a pharmaceutically acceptable salt thereof, wherein Ar¹ is optionally substituted thiophenyl or optionally substituted thiazolyl.

128. The compound according to any of claims 124-127, or a pharmaceutically acceptable salt thereof, wherein R^{1b} is optionally substituted 5- or 6-membered heteroaryl.

129. The compound according to any of claims 124-127, or a pharmaceutically acceptable salt thereof, wherein R^{1b} is CONH(CH₃) or cyano.

130. The compound according to any of claims 124-129, or a pharmaceutically acceptable salt thereof, wherein R^{1c} is hydrogen or OCH₃.

131. The compound according to any of claims 124-130, or a pharmaceutically acceptable salt thereof, wherein R^{5a} is C(O)CH₃, CH₃, or C(O)NH₂.

132. A compound, wherein said compound is a compound of Formula VA, Formula VB, Formula VC, or Formula VD: 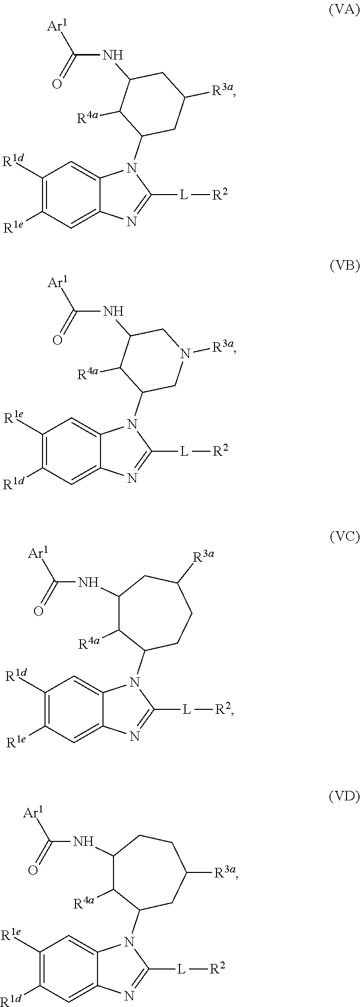
or a pharmaceutically acceptable salt thereof, wherein:
L is a bond, —O—, —NR^{a}—, or optionally substituted C₁-C₄alkylene;
R^{a} is hydrogen or C₁-C₄alkyl;
R^{1d} is optionally substituted heteroaryl, optionally substituted aryl, optionally substituted 4-, 5- or 6-membered heterocycloalkyl, CONR^{j}(C₁-C₄alkyl), CONR^{j}(C₃-C₄cycloalkyl), or cyano;
each R^{j} is independently hydrogen or C₁-C₄alkyl;
R^{1e} is hydrogen, halo, C₁-C₆alkyl, C₁-C₆haloalkyl, C₁-C₆alkoxy, or C₁-C₆haloalkoxy;
R² is optionally substituted phenyl, optionally substituted pyridinyl, optionally substituted 5-membered heteroaryl, optionally substituted heterocycloalkyl, optionally substituted C₁-C₆alkyl, optionally substituted C₁-C₆alkoxy, optionally substituted C₁-C₆hydroxyalkyl, optionally substituted C₁-C₆haloalkyl, optionally substituted C₁-C₆haloalkoxy, optionally substituted C₃-C₆cycloalkyl, or NR^{f}R^{g};
R^{f} and R^{g} are each independently hydrogen, C₁-C₆alkyl, C₁-C₆haloalkyl, OH, C₁-C₄alkoxy, C₁-C₆hydroxyalkyl, or C₃-C₆cycloalkyl;
one of R^{3a} or R^{4a} is hydrogen, deuterium, OH, cyano, C₁-C₃alkyl, C₁-C₃haloalkyl, C₁-C₃alkoxy, C₁-C₃haloalkoxy, deuterated C₁-C₃alkoxy, NH₂, NH(C₁-C₄alkyl), N(C₁-C₄alkyl)₂, OCO₂(C₁-C₄alkyl), OCO₂(C₃-C₄cycloalkyl), OSO₂(C₁-C₄alkyl), OSO₂(C₃-C₄cycloalkyl), OC(O)(C₁-C₄alkyl), OC(O)(C₃-C₄cycloalkyl), or NR^{m}Rⁿ; and
the other of R^{3a} or R^{4a} is OH, cyano, C₁-C₃alkyl, C₁-C₃haloalkyl, C₁-C₃alkoxy, C₁-C₃haloalkoxy, deuterated C₁-C₃alkoxy, NH₂, NH(C₁-C₄alkyl), N(C₁-C₄alkyl)₂, OCO₂(C₁-C₄alkyl), OSO₂(C₃-C₄cycloalkyl), OSO₂(C₁-C₄alkyl), OSO₂(C₃-C₄cycloalkyl), OC(O)(C₁-C₄alkyl), OC(O)(C₃-C₄cycloalkyl), or NR^{m}Rⁿ; and wherein each R^{m} is independently hydrogen or C₁-C₄alkyl and each Rⁿ is independently CO₂(C₁-C₄alkyl), C(O)C₁-C₄alkyl, SO₂(C₁-C₄alkyl), or SO₂(C₃-C₄cycloalkyl); and
Ar¹ is an optionally substituted five-membered heteroaryl.

133. The compound according to claim 132, or a pharmaceutically acceptable salt thereof, wherein the compound is a compound of Formula VA or Formula VB.

134. The compound according to claim 133, wherein the compound of Formula VA is a compound of Formula VA-1, or a pharmaceutically acceptable salt thereof, and the compound of Formula VB is a compound of Formula VB-1, or a pharmaceutically acceptable salt thereof:

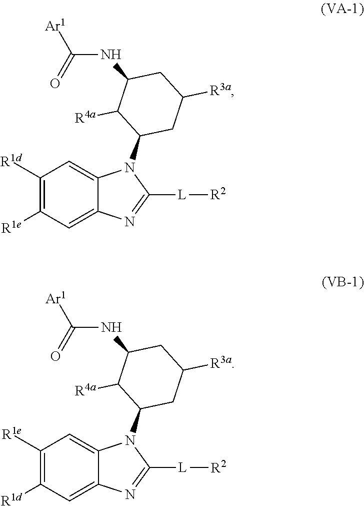

135. The compound according to claim 132, or a pharmaceutically acceptable salt thereof, wherein the compound is a compound of Formula VC or Formula VD.

136. The compound according to claim 135, wherein the compound of Formula VC is a compound of Formula VC-1, or a pharmaceutically acceptable salt thereof, and the compound of Formula VD is a compound of Formula VD-1, or a pharmaceutically acceptable salt thereof:

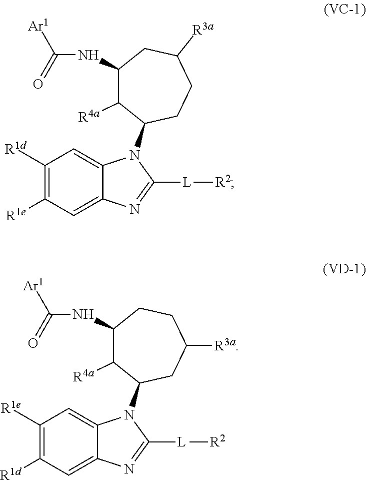

137. The compound according to any one of claims 132-136, or a pharmaceutically acceptable salt thereof, wherein one of R^{3a} or R^{4a} is OH or C₁-C₃alkoxy; and
the other of R^{3a} or R^{4a} is hydrogen.

138. The compound according to any one of claims 132-137, or a pharmaceutically acceptable salt thereof, wherein L is a bond and R² is optionally substituted phenyl or optionally substituted pyridinyl.

139. The compound according to any one of claims 132-137, or a pharmaceutically acceptable salt thereof, where L is a bond and R² is phenyl, substituted with one or more of halo, C₁-C₄alkyl, C₃-C₄cycloalkyl, C₁-C₄alkoxy, C₁-C₄haloalkoxy, cyano, OH, NR^{b}R^{c}, C(O)NR^{b}R^{c}, CO₂C₁-C₄alkyl, COOH, C₁-C₄alkylene-R²⁹, or O—C₁-C₄alkylene-R³⁰;
R²⁹ is cyano, NR^{b}R^{c}, or COOH;
R³⁰ is OH, OCH₃, or NR^{b}R^{c}; and
each R^{b} and each R^{C} is independently hydrogen or C₁-C₄alkyl.

140. The compound according to any one of claims 132-139, or a pharmaceutically acceptable salt thereof, wherein Ar¹ is optionally substituted thiophenyl or optionally substituted thiazolyl.

141. The compound according to any one of claims 132-140, or a pharmaceutically acceptable salt thereof, wherein R^{1d} is C(O)NH(CH₃) and R^{1e} is hydrogen.

142. The compound according to any one of claims 132-140, or a pharmaceutically acceptable salt thereof, wherein R^{1d} is optionally substituted 5- or 6-membered heteroaryl and R^{1e} is hydrogen.

143. The compound according to claim 142, or a pharmaceutically acceptable salt thereof, wherein R^{1d} is optionally substituted oxadiazolyl, optionally substituted thiadiazolyl, optionally substituted oxazolyl, optionally substituted pyrazolyl, optionally substituted imidazolyl, optionally substituted triazolyl, optionally substituted tetrazolyl, optionally substituted thiazolyl, or optionally substituted pyridinyl, optionally substituted pyrimidinyl, or optionally substituted pyridazinyl.

144. The compound according to claim 142, or a pharmaceutically acceptable salt thereof, wherein R^{1d} is optionally substituted oxazolyl, optionally substituted triazolyl, optionally substituted pyrimidinyl, or optionally substituted thiazolyl.

145. The compound of claim 1 that is a compound of Table 1, or a pharmaceutically acceptable salt thereof.

146. The compound of claim 108 that is a compound of Table 2, or a pharmaceutically acceptable salt thereof.

147. The compound of claim 115 that is a compound of Table 3, or a pharmaceutically acceptable salt thereof.

148. The compound of claim 124 that is a compound of Table 4, or a pharmaceutically acceptable salt thereof.

149. The compound of claim 132 that is a compound of Table 5, or a pharmaceutically acceptable salt thereof.

150. A pharmaceutical composition comprising a compound according to any one of claims 1-149, or a pharmaceutically acceptable salt thereof, and a pharmaceutically acceptable excipient.

151. A method of reducing plasma methylmalonic acid in a subject having an elevated methylmalonic acid level comprising administering to the subject a therapeutically effective amount of a compound of any one of claims 1-149.

152. A method of treating a BCAA-catabolism-related diseases in a subject in need thereof comprising administering to the subject a therapeutically effective amount of a compound, or a pharmaceutically acceptable salt thereof, of any one of claims 1-149.

153. The method according to claim 152, wherein the BCAA-catabolism-related disease is methylmalonic acidemia (MMA), propionic acidemia (PA), isovaleric acidemia (IVA), maple syrup urine disease, methylmalonic semialdehyde (MMSDH) deficiency, 3-hydroxyisobutyrate dehydrogenase (HIBADH) deficiency, 3-hydroxyisobutyryl-CoA hydrolase (HIBCH) deficiency, isobutyryl-CoA (IBD) deficiency, acetyl-CoA carboxylase 1 (ACC) deficiency, mitochondrial short-chain enoyl-CoA hydratase 1 (ECHS1) deficiency, methylbutyryl CoA dehydrogenase (SBCAD) deficiency, beta-ketothiolase deficiency (BKT), hydroxy-3-methylglutaconyl-CoA lysase (HMGCL) deficiency, 3-methylglutaconyl-CoA-hydratase deficiency, or 3-methylcrotonyl-CoA (3-MCC) deficiency.

154. A method of reducing blood ammonia in a subject having an elevated ammonia level comprising administering to the subject a therapeutically effective amount of a compound of any one of claims 1-149.

155. A method of reducing blood 2-methylcitrate in a subject having an elevated 2-methylcitrate level comprising administering to the subject a therapeutically effective amount of a compound of any one of claims 1-149.


SEQUENCE LISTING (2 sequences)

SEQ ID NO: 1 - DNA, 20 bp, synthetic construct
  misc_feature 1..20: note = Synthetic: guide sequence
  source 1..20: mol_type = other DNA
atcatgtaagaacctcccca

SEQ ID NO: 2 - DNA, 20 bp, synthetic construct
  misc_feature 1..20: note = Synthetic: guide sequence
  source 1..20: mol_type = other DNA
tactggataaatgaagatgg